SEQUENCE LISTING (2 sequences)

SEQ ID NO: 1 - DNA, 20 bp, Artificial Sequence
  Synthetic Polynucleotide
aatgactgaatataaacttg

SEQ ID NO: 2 - DNA, 198 bp, Artificial Sequence
  Synthetic Polynucleotide
attaaccttatgtgtgacatgttctaatatagtcacattttcattatttttattataaggcgtgctgaaaatggtgagcggctggcggctgttcaagaagattagcactgaatataaacttgtagtcgtaggtgctgatggcgtaggcaagagtgccttgacgatacagctaattcagaatcattttgtggacgaata

(19) United States
(12) Patent Application Publication
(10) Pub. No.: US 2024/0293558 A1
(43) Pub. Date: Sep. 5, 2024

(54) KRAS INHIBITOR CONJUGATES

(71) Applicant: Erasca, Inc., San Diego, CA (US)

(72) Inventors: Hengmiao CHENG, San Diego, CA (US); Jean-Michel VERNIER, San Diego, CA (US); Ping CHEN, San Diego, CA (US)

(21) Appl. No.: 18/570,993

(22) PCT Filed: Jun. 15, 2022
(86) PCT No.: PCT/US2022/033602

§ 371 Date: Dec. 15, 2023

Related U.S. Application Data
(60) Provisional application No. 63/211,546, filed on Jun. 16, 2021.
(60) Provisional application No. 63/212,070, filed on Jun. 17, 2021.
(60) Provisional application No. 63/232,572, filed on Aug. 12, 2021.
(60) Provisional application No. 63/250,154, filed on Sep. 29, 2021.
(60) Provisional application No. 63/270,967, filed on Oct. 22, 2021.
(60) Provisional application No. 63/291,912, filed on Dec. 20, 2021.
(60) Provisional application No. 63/302,958, filed on Jan. 25, 2022.
(60) Provisional application No. 63/352,067, filed on Jun. 14, 2022.

(51) Int. Cl. A61K 47/55; A61K 47/54

(52) U.S. Cl. CPC A61K 47/55; A61K 47/545

(57) ABSTRACT

The present application provides conjugates of protein-binding ligands, in particular G12D mutant KRAS ligands, with ubiquitin ligase ligands and methods for treating diseases such as cancer.

### REFERENCE TO SEQUENCE LISTING

This application incorporates by reference a Computer Readable Form (CRF) of a Sequence Listing in ASCII text format submitted with this application, entitled 055745-532001WO_SL_ST25.TXT, was created on Jun. 15, 2022, and is 1,175 bytes in size.

### BACKGROUND

Embodiments herein relate to compounds, compositions and methods for the treatment of RAS-mediated disease. In particular, embodiments herein relate to compounds and methods for treating diseases such as cancer via targeting oncogenic mutants of the K-RAS isoform.

Ras proteins are small guanine nucleotide-binding proteins that act as molecular switches by cycling between active GTP-bound and inactive GDP-bound conformations. Ras signaling is regulated through a balance between activation by guanine nucleotide exchange factors (GEFs), most commonly son of sevenless (SOS), and inactivation by GTPase-activating proteins (GAPs) such as neurofibromin or p120GAP. The Ras proteins play an important role in the regulation of cell proliferation, differentiation, and survival. Dysregulation of the Ras signaling pathway is almost invariably associated with disease. Hyper-activating somatic mutations in Ras are among the most common lesions found in human cancer. Most of these mutations have been shown to decrease the sensitivity of Ras to GAP stimulation and decrease its intrinsic GTPase activity, leading to an increase in the active GTP-bound population. Although mutation of any one of the three Ras isoforms (K-Ras, N-Ras, or H-Ras) has been shown to lead to oncogenic transformation, K-Ras mutations are by far the most common in human cancer. For example, K-Ras mutations are known to be often associated with pancreatic, colorectal and non-small-cell lung carcinomas. Similarly, H-Ras mutations are common in cancers such as papillary thyroid cancer, lung cancers and skin cancers. Finally, N-Ras mutations occur frequently in hepatocellular carcinoma.

K-Ras is the most frequently mutated oncoprotein in human cancers, and the G12D mutation is among the most prevalent. Accordingly, there is a need to develop selective inhibitors of KRAS G12D. The present embodiments meet this and other needs.

### SUMMARY

In one aspect, the present embodiments provide conjugates, or a pharmaceutically acceptable salt thereof, of Formula(A):

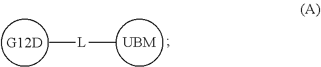

wherein G12D is a KRAS inhibitor capable of binding to a KRAS protein having a G12D mutation; L is a bivalent linker that connects G12D to a ubiquitin binding moiety (UBM); and wherein UBM binds to a ubiquitin ligase.

In another aspect, the present embodiments provide a pharmaceutical composition comprising a pharmaceutically effective amount of the conjugates disclosed herein, or a pharmaceutically acceptable salt thereof, and a pharmaceutically acceptable excipient.

In another embodiment, the present embodiments provide a method of treating a subject having cancer, the cancer characterized by the presence of a KRAS G12D mutation, the method comprising administering to the subject a therapeutically effective amount of a conjugates disclosed herein, or a pharmaceutically acceptable salt thereof, or a pharmaceutical composition as disclosed herein.

In another embodiment, the present embodiments provide a method for manufacturing a medicament for treating a subject having cancer, the cancer characterized by the presence of a KRAS G12D mutation, the medicament comprising a conjugate disclosed herein, or a pharmaceutically acceptable salt thereof, or a a pharmaceutical composition as disclosed herein, is used.

In another embodiment, the present embodiments provide for the use of a conjugate disclosed herein, or a pharmaceutically acceptable salt thereof, or a a pharmaceutical composition as disclosed herein, for the manufacture of a medicament for the treatment of cancer in a subject, the cancer characterized by the presence of a KRAS G12D mutation.

In another embodiment, the present embodiments provide the conjugates disclosed herein, or a pharmaceutically acceptable salt thereof, or a a pharmaceutical composition as disclosed herein, for use in the treatment of cancer in a subject, the cancer characterized by a KRAS G12D mutation.

### BRIEF DESCRIPTION OF THE DRAWINGS

FIG. 1 shows a Western blot indicating the efficacy of KRAS G12D degradation in the presence of the conjugate of Example 4, in accordance with embodiments herein.

FIG. 2 shows a Western blot indicating the efficacy of KRAS G12D degradation in the presence of the conjugate of Example 6, in accordance with embodiments herein. AsPc-1 cell line after 24 h treatment. DC₅₀ is determined to be 255 nM, Dmax is 95%.

FIG. 3 shows a Western blot indicating the efficacy of KRAS G12D degradation in the presence of the conjugate of Example 7, in accordance with embodiments herein. AsPc-1 cell line after 24 h treatment.

FIG. 4 shows a Western blot indicating the efficacy of KRAS G12D degradation in the presence of the conjugate of Example 8, in accordance with embodiments herein. AsPc-1 cell line after 24 h treatment. DC₅₀ is determined to be 71 nM, Dmax is 95%.

FIG. 5 shows a Western blot indicating the efficacy of KRAS G12D degradation in the presence of the conjugate of Example 9, in accordance with embodiments herein. AsPc-1 cell line after 24 h treatment. DC₅₀ is determined to be 183 nM, Dmax is 95%.

FIG. 6 shows a Western blot indicating the efficacy of KRAS G12D degradation in the presence of the conjugate of Example 12, in accordance with embodiments herein. AsPc-1 cell line after 24 h treatment.

FIG. 7 shows a Western blot indicating the efficacy of KRAS G12D degradation in the presence of the conjugate of Example 13, in accordance with embodiments herein. AsPc-1 cell line after 24 h treatment.

FIG. 8 shows a Western blot indicating the efficacy of KRAS G12D degradation in the presence of the conjugate of Example 14, in accordance with embodiments herein. AsPc-1 cell line after 24 h treatment.

FIG. 9 shows a Western blot indicating the degradation assay for Example 200.

FIG. 10 shows a Western blot indicating the degradation assay for Example 201.

FIG. 11 shows KRAS wild type degradation of Example 20 in ASPC-1 cell line after 24 h treatment. DC₅₀=8.2 nM.

FIG. 12 shows KRAS wild type degradation of Example 20 in HT-29 cell line, after 24 h treatment. DC₅₀=426 nM.

### DETAILED DESCRIPTION

### I. GENERAL

The present embodiments provide conjugates of selective inhibitors of KRAS G12D exhibiting good selectivity over wild-type KRAS conjugated to ubiquitin binding moieties and are useful for treating a cancer characterized by a KRAS G12D mutation.

### II. DEFINITIONS

Unless specifically indicated otherwise, all technical and scientific terms used herein have the same meaning as commonly understood by those of ordinary skill in the art to which the embodiments belong. In addition, any method or material similar or equivalent to a method or material described herein can be used in the practice of the present embodiments. For purposes of the present embodiments, the following terms are defined.

“A,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The following chemical functional group definitions are provided to give guidance in understanding their meaning and scope. Those skilled in the art will recognize that these functional groups are being used in a manner consistent with practice of the chemical arts. Any of the following chemical functional groups may be optionally substituted as defined below and each chemical functional group below may itself be an optional substitution.

The term “acyl,” as used herein, alone or in combination, refers to a carbonyl (C═O) attached to an alkenyl, alkyl, aryl, cycloalkyl, heteroaryl, heterocycloalkyl, or any other moiety were the atom attached to the carbonyl is carbon. An “acetyl” group, which is a type of acyl, refers to a (—C(═O)CH₃) group. An “alkylcarbonyl” or “alkanoyl” group refers to an alkyl group attached to the parent molecular moiety through a carbonyl group. Examples of such groups include, without limitation, methylcarbonyl and ethylcarbonyl. Similarly, an “arylcarbonyl” or “aroyl” group refers to an aryl group attached to the parent molecular moiety through a carbonyl group. Examples of such groups include, without limitation, benzoyl and naphthoyl. Accordingly, generic examples of acyl groups include alkanoyl, aroyl, heteroaroyl, and so on. Specific examples of acyl groups include, without limitation, formyl, acetyl, acryloyl, benzoyl, trifluoroacetyl and the like.

The term “alkenyl,” as used herein, alone or in combination, refers to a straight-chain or branched-chain hydrocarbon radical having one or more double bonds and containing from 2 to 20 carbon atoms. In certain embodiments, the alkenyl may comprise from 2 to 6 carbon atoms, or from 2 to 4 carbons, either of which may be referred to as “lower alkenyl.” The term “alkenylene” refers to a carbon-carbon double bond system attached at two or more positions such as ethenylene (—CH═CH—). Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆, and so on up to 20 carbon atoms. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted. Unless otherwise specified, the term “alkenyl” may include “alkenylene” groups.

The term “alkoxy,” as used herein, alone or in combination, refers to an alkyl ether radical, wherein the term alkyl is as defined below. Alkoxy groups may have the general formula: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, and the like. The alkoxy groups can be further optionally substituted as defined herein.

The term “alkyl,” as used herein, alone or in combination, (sometimes abbreviated Alk) refers to a straight-chain or branched-chain alkyl radical containing from 1 to 20 carbon atoms. In certain embodiments, the alkyl may comprise from 1 to 10 carbon atoms. In further embodiments, the alkyl may comprise from 1 to 6 carbon atoms, or from 1 to 4 carbon atoms. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups. When the alkyl is methyl, it may be represented structurally as CH₃, Me, or just a single bond terminating with no end group substitution.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino (—NHMe), N-ethylamino (—NHEt), N,N-dimethylamino (—NMe₂), N,N-ethylmethylamino (—NMeEt) and the like. The term “aminoalkyl” refers to reverse orientation in which the amino group appears distal to the parent molecular moiety and attachment to the parent molecular moiety is through the alkyl group. For example, NH₂(CH₂)ₙ-describes an aminoalkyl group with a terminal amine at the end of an alkyl group attached to the parent molecular moiety. The two terms alkylamino and aminoalkyl can be combined to describe an “alkylaminoalkyl” group in which an alkyl group resides on a nitrogen atom distal to the parent molecular moiety, such as MeNH(CH₂)ₙ—. In a similar manner, an aryl group, as defined herein, may combine in a similar fashion providing an arylaminoalkyl group ArNH(CH₂)ₙ—. For additional clarity nomenclature may be provided where the group that is attached to nitrogen is indicated so by use of “N—” in the name, such as N-arylaminoalkyl, which is understood to mean that the aryl group is a substituent on the nitrogen atom of the aminoalkyl group, the alkyl being attached the parent molecular moiety.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (AlkS-) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like. Similarly, “arylthio” refers to arylthioether (ArS—) radical wherein the term aryl is as defined herein and wherein the sulfur may be singly or double oxidized.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted. Unless otherwise specified, the term “alkynyl” may include “alkynylene” groups.

The terms “amido,” as used herein, alone or in combination, refer to an amino group as described below attached to the parent molecular moiety through a carbonyl group. The term “C-amido” as used herein, alone or in combination, refers to a —C(═O)N(R)₂ group where is R as defined herein. The term “N-amido” as used herein, alone or in combination, refers to RC(═O)N(R′)— group, with R and R′ as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —N(R)(R′) or —N⁺(R)(R′)(R″), wherein R, R′ and R″ are independently selected from the group consisting of hydrogen, alkyl, acyl, heteroalkyl, aryl, cycloalkyl, heteroaryl, and heterocycloalkyl, any of which may themselves be optionally substituted.

The term “amino acid,” as used herein, alone or in combination, means a substituent of the form —NRCH(R′)C(O)OH, wherein R is typically hydrogen, but may be cyclized with N (for example, as in the case of the amino acid proline), and R′ is selected from the group consisting of hydrogen, alkyl, heteroalkyl, cycloalkyl, heterocycloalkyl, aryl, heteroaryl, amino, amido, cycloalkylalkyl, heterocycloalkylalkyl, arylalkyl, heteroarylalkyl, aminoalkyl, amidoalkyl, hydroxyalkyl, thiol, thioalkyl, alkylthioalkyl, and alkylthio, any of which may be optionally substituted. The term “amino acid” includes all naturally occurring amino acids as well as synthetic analogues.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, naphthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄— derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid (oxygen) end, and which may be optionally substituted as defined herein.

The term “O-carbamyl” as used herein, alone or in combination, refers to a —OC(O)NRR′, group, with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(═O)H] and in combination is a —C(═O)— group.

The term “carboxyl” or “carboxyl,” as used herein, refers to —C(═O)OH, O-carboxy, C-carboxy, or the corresponding “carboxylate” anion, such as is in a carboxylic acid salt. An “O-carboxy” group refers to a RC(═O)O— group, where R is as defined herein. A “C-carboxy” group refers to a —C(═O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl radical wherein each cyclic moiety contains from 3 to 12 carbon atom ring members and which may optionally be a benzo fused ring system which is optionally substituted as defined herein. In some embodiments, a cycloalkyl may comprise from from 3 to 7 carbon atoms, or from 5 to 7 carbon atoms. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1.1.1]pentane, camphor, adamantane, and bicyclo[3.2.1]octane.

The term “ester,” as used herein, alone or in combination, refers to a carboxyl group bridging two moieties linked at carbon atoms (—CRR′C(═O)OCRR′—), where each R and R′ are independent and defined herein.

The term “ether,” as used herein, alone or in combination, typically refers to an oxy group bridging two moieties linked at carbon atoms. “Ether” may also include polyethers, such as, for example, —RO(CH₂)₂O(CH₂)₂O(CH₂)₂OR′, —RO(CH₂)₂O(CH₂)₂OR′, —RO(CH₂)₂OR′, and —RO(CH₂)₂OH.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl, trihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized (i.e. bond to 4 groups). The heteroatom(s) O, N and S may be placed at any interior position of the heteroalkyl group. Up to two heteroatoms may be consecutive, such as, for example, —CH₂NHOCH₃. The term heteroalkyl may include ethers.

The term “heteroaryl,” as used herein, alone or in combination, refers to 3 to 7 membered unsaturated heteromonocyclic rings, or fused polycyclic rings, each of which is 3 to 7 membered, in which at least one of the fused rings is unsaturated, wherein at least one atom is selected from the group consisting of O, S, and N. In some embodiments, a heteroaryl may comprise from 5 to 7 carbon atoms. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Non-limiting examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

Heteroaryl groups can include any number of ring atoms, such as, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

The terms “heterocycloalkyl” and, interchangeably, “heterocycle,” or “heterocyclyl” as used herein, alone or in combination, each refer to a saturated, partially unsaturated, or fully unsaturated monocyclic, bicyclic, spirocyclic, or tricyclic heterocyclic radical containing at least one heteroatom as ring members, wherein each heteroatom may be independently selected from the group consisting of nitrogen, oxygen, and sulfur. In certain embodiments, a heterocycloalkyl may comprise from 1 to 4 heteroatoms as ring members. In further embodiments, a heterocycloalkyl may comprise from 1 to 2 heteroatoms ring members. In some embodiments, a heterocycloalkyl may comprise from 3 to 8 ring members in each ring. In further embodiments, a heterocycloalkyl may comprise from 3 to 7 ring members in each ring. In yet further embodiments, a heterocycloalkyl may comprise from 5 to 6 ring members in each ring. “Heterocycloalkyl” and “heterocycle” are intended to include sugars, sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycloalkyl groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, epoxy, isoindolinyl, morpholinyl, piperazinyl, pyrrolidinyl, tetrahydropyridinyl, piperidinyl, thiomorpholinyl, hexahydro-1H-pyrrolizine and the like. The heterocycloalkyl groups may be optionally substituted unless specifically prohibited.

“Heterocycloalkyl” may refer to a saturated ring system having from 3 to 12 ring members and from 1 to 5 heteroatoms of N, O and S. The heteroatoms can also be oxidized, such as, but not limited to, S(O) and S(O)₂. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, 4, or 5, or 1 to 2, 1 to 3, 1 to 4, 1 to 5, 2 to 3, 2 to 4, 2 to 5, 3 to 4 or 3 to 5. The heterocycloalkyl group can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. The heterocycloalkyl group can include groups such as aziridine, azetidine, pyrrolidine, piperidine, azepane, diazepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline, diazabicycloheptane, diazabicyclooctane, diazaspirooctane or diazaspirononane. Heterocycloalkyl groups can be unsubstituted or substituted. For example, heterocycloalkyl groups can be substituted with C1 6 alkyl or oxo (═O), among many others. Heterocycloalkyl groups can also include a double bond or a triple bond, such as, but not limited to dihydropyridine or 1,2,3,6-tetrahydropyridine.

The heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, morpholine can be 2-, 3- or 4-morpholine, and hexahydro-1H-pyrrolizine can be 1-, 2-, 3-, 5-, 6-, 7-, 7a-hexahydro-1H-pyrrolizine.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxzoalidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

The term “hydrazinyl” as used herein, alone or in combination, refers to two amino groups joined by a single bond, i.e., —N—N—. In general, the hydrazinyl group has optional substitution on at least one NH hydrogen to confer stability.

The term “hydroxamic acid” or its ester as used herein, refers to —C(O)ON(R)O(R′), wherein R and R′ are as defined herein, or the corresponding “hydroxamate” anion, including any corresponding hydroxamic acid salt.

The term “hydroxy,” as used herein, alone or in combination, refers to OH.

The term “hydroxyalkyl,” as used herein, alone or in combination, refers to a hydroxy group attached to the parent molecular moiety through an alkyl group. “Hydroxyalkyl” or “alkylhydroxy” refers to an alkyl group, as defined above, where at least one of the hydrogen atoms is replaced with a hydroxy group. As for the alkyl group, hydroxyalkyl or alkylhydroxy groups can have any suitable number of carbon atoms, such as C₁₋₆. Exemplary C₁₋₄ hydroxyalkyl groups include, but are not limited to, hydroxymethyl, hydroxyethyl (where the hydroxy is in the 1 or 2 position), hydroxypropyl (where the hydroxy is in the 1, 2 or 3 position), hydroxybutyl (where the hydroxy is in the 1, 2, 3 or 4 position), 1,2dihydroxyethyl, and the like.

The term “imino,” as used herein, alone or in combination, refers to C═NR.

The term “iminohydroxy,” as used herein, alone or in combination, refers to C═N(OH) and it O-ether C═N—OR.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “linking group,” as used herein refers to any nitrogen containing organic fragment that serves to connect the pyrimidine or pyridone core of the compounds disclosed herein to the electrophilic moiety E, as defined herein. Exemplary linking groups include piperazines, aminoalkyls, alkyl- or aryl-based diamines, aminocycloalkyls, amine-containing spirocyclics, any of which may be optionally substituted as defined herein. In some embodiments, linking groups may comprise the substructure L-Q-L′-E wherein Q is a monocyclic 4 to 7 membered ring or a bicyclic, bridged, or fused, or spiro 6-11 membered ring, any of which optionally include one or more nitrogen atoms, E is the electrophilic group, L is bond, C₁₋₆ alkylene, —O—C₀₋₅ alkylene, —S—C₀₋₅ alkylene, or —NH—C₀₋₅ alkylene, and for C₂₋₆ alkylene, —O—C₂₋₅ alkylene, —S—C₂₋₅ alkylene, and NH—C₂₋₅ alkylene, one carbon atom of any of the alkylene groups can optionally be replaced with O, S, or NH; and L′ is bond when Q comprises a nitrogen to link to E, otherwise L′ is NR, where R is hydrogen or alkyl.

The term “lower,” as used herein, alone or in combination, means containing from 1 to and including 6 carbon atoms, or from 1 to 4 carbon atoms.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “phosphoamide” as used herein, alone or in combination, refers to a phosphate group [(OH)₂P(═O)O—] in which one or more of the hydroxyl groups has been replaced by nitrogen, amino, or amido.

The term “phosphonate” as used herein, alone or in combination, refers to a group of the form ROP(OR′)(OR)O— wherein R and R′ are selected from the group consisting of hydrogen, alkyl, acyl, heteroalkyl, aryl, cycloalkyl, heteroaryl, and heterocycloalkyl, any of which may themselves be optionally substituted. “Phosphonate” includes “phosphate [(OH)₂P(O)O—] and related phosphoric acid anions which may form salts.

The terms “sulfonate,” “sulfonic acid,” and “sulfonic,” as used herein, alone or in combination, refers to the —SO₃H group and its anion as the sulfonic acid is used in salt formation or sulfonate ester where OH is replaced by OR, where R is not hydrogen, but otherwise is as defined herein, and typically being alkyl or aryl.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(═S)H and in combination is a —C(═S)— group.

The term “N-thiocarbamyl” refers to an ROC(═S)NR′— group, with R and R′ as defined herein.

The term “O-thiocarbamyl” refers to a —OC(═S)NRR′, group with R and R′ as defined herein.

The term “thiocyanato” refers to a —CNS group.

The term “trihalomethanesulfonamido” refers to a X₃CS(═O)₂NR— group with X is a halogen and R as defined herein.

The term “trihalomethanesulfonyl” refers to a X₃CS(═O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent. A “null” group occurring between two other group may also be understood to be a collapsing of flanking groups. For example, if in —(CH₂)ₓG¹G²G³, the element G² were null, said group would become —(CH₂)ₓG¹G³.

The term “optionally substituted” means the anteceding group or groups may be substituted or unsubstituted. Groups constituting optional substitution may themselves be optionally substituted. For example, where an alkyl group is embraced by an optional substitution, that alkyl group itself may also be optionally substituted. When substituted, the substituents of an “optionally substituted” group may include, without limitation, one or more substituents independently selected from the following groups or a particular designated set of groups, alone or in combination: alkyl, alkenyl, alkynyl, alkanoyl, heteroalkyl, heterocycloalkyl, haloalkyl, haloalkenyl, haloalkynyl, lower perhaloalkyl, perhaloalkoxy, cycloalkyl, phenyl, aryl, aryloxy, alkoxy, haloalkoxy, oxo, acyloxy, carbonyl, carboxyl, alkylcarbonyl, carboxyester, carboxamido, cyano, hydrogen, halogen, hydroxy, amino, alkylamino, arylamino, amido, nitro, thiol, alkylthio, haloalkylthio, perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, carbamate, and urea. Particular subsets of optional substitution include, without limitation: (1) alkyl, halo, and alkoxy; (2) alkyl and halo; (3) alkyl and alkoxy; (4) alkyl, aryl, and heteroaryl; (5) halo and alkoxy; and (6) hydroxyl, alkyl, halo, alkoxy, and cyano. Where an optional substitution comprises a heteroatom-hydrogen bond (—NH—, SH, OH), further optional substitution of the heteroatom hydrogen is contemplated and includes, without limitation optional substitution with alkyl, acyl, alkoxymethyl, alkoxyethyl, arylsulfonyl, alkyl sulfonyl, any of which are further optionally substituted. These subsets of optional substitutions are intended to be merely exemplary and any combination of 2 to 5, or 2 to 10, or 2 to 20 of the groups recited above up to all the group recited above and any subrange in between are contemplated. “Optionally substituted” may include any of the chemical functional groups defined hereinabove and throughout this disclosure. Two optional substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃).

The various optional substitutions need not be the same and any combination of optional substituent groups may be combined. For example, a carbon chain may be substituted with an alkyl group, a halo group, and an alkoxy group. Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, hydroxyl, halogen, alkyl, cycloalkyl, heteroalkyl, aryl, heteroaryl and heterocycloalkyl, any of which may be optionally substituted. Each such R and R′ groups should be understood to be optionally substituted as defined herein. Each incidence of R and R′ should be understood to be independent. Whether an R group has a number designation or not, every R group, including R, R′ and R″ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

The groups defined above can optionally be substituted by any suitable number and type of subsituents. Representative substituents include, but are not limited to, halogen, haloalkyl, haloalkoxy, —OR′, ═O, —OC(O)R′, —(O)R′, —O₂R′, —ONR′R″, —OC(O)NR′R″, ═NR′, N—OR′, —NR′R″, —NR″C(O)R′, —NR′—(O)NR″R″′, —NR″C(O)OR′, —NH—(NH₂)═NH, —NR′C(NH₂)═NH, —NH—(NH₂)═NR′, —SR′, —S(O)R′, —S(O)₂R′, —S(O)₂NR′R″, —NR'S(O)₂R″, —N₃ and —NO₂. R′, R″ and R″′ each independently refer to hydrogen, unsubstituted alkyl, such as unsubstituted C₁₋₆ alkyl. Alternatively, R′ and R″, or R″ and R″′, when attached to the same nitrogen, are combined with the nitrogen to which they are attached to form a heterocycloalkyl or heteroaryl ring, as defined above.

“Conjugate” refers to compounds disclosed herein that are constructed by linking two components, a binder of KRAS having the G12D mutation and ubiquitin binding moiety. The term “conjugate” and “compound” may be used interchangeably.

“Ubiquitin binding moiety,” or “UBM,” refers to a portion of the conjugates, as set forth herein, that is capable of binding to an E3 ubiquitin ligase. In embodiments, the UBM is a monovalent form of a E3 ubiquitin ligase ligand that is covalently bonded in the conjugate. In embodiments, the UBM is a divalent form of a E3 ubiquitin ligase ligand that is integrated into the conjugate. The substrate recognition subunits of E3 ubiquitin ligases include, for example, Von Hippel-Lindau (VHL), cereblon (CRBN), inhibitor of apoptosis (IAP), and mouse double minute 2 homolog (MDM2) ligases.

“Salt” refers to acid or base salts of the compounds, which can be used in the methods disclosed herein. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds disclosed herein are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present embodiments.

Certain compounds disclosed herein possess asymmetric carbon atoms (optical centers) or double bonds; the racemates, diastereomers, geometric isomers and individual isomers are all intended to be encompassed within the scope of the present embodiments.

“Hydrate” refers to a compound that is complexed to at least one water molecule. The compounds disclosed herein can be complexed with from 1 to 10 water molecules.

“Composition” as used herein is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and deleterious to the recipient thereof.

“Pharmaceutically acceptable excipient” refers to a substance that aids the administration of an active agent to and absorption by a subject. Pharmaceutical excipients useful in the present embodiments include, but are not limited to, binders, fillers, disintegrants, lubricants, coatings, sweeteners, flavors and colors. One of skill in the art will recognize that other pharmaceutical excipients are useful in the present embodiments.

“Treat”, “treating” and “treatment” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom. The treatment or amelioration of symptoms can be based on any objective or subjective parameter; including, e.g., the result of a physical examination.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Subject” refers to animals such as mammals, including, but not limited to, primates (e.g., humans), cows, sheep, goats, horses, dogs, cats, rabbits, rats, mice and the like. In certain embodiments, the subject is a human.

### III. COMPOUNDS

The present embodiments provide compounds, and pharmaceutically acceptable salts thereof, of Formula (A):

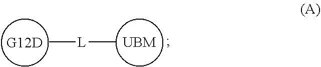

wherein G12D is a KRAS inhibitor capable of binding to a KRAS protein having a G12D mutation; L is a linker that connects G12D to a ubiquitin binding moiety (UBM); and wherein UBM binds to a ubiquitin ligase.

In embodiments, the conjugate is a structure of Formula (I) or Formula (II):

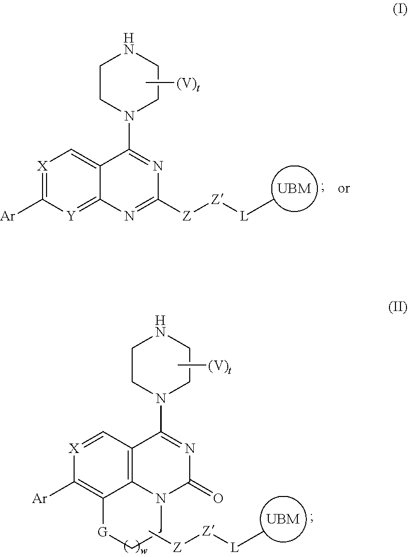

wherein t is integer from 0 to 4;
w is 1 or 2;
Ar is selected from 6- to 10-membered aryl, 5-10 membered heteroaryl; wherein the 5-10 membered heteroaryl ring contains 1 to 4 heteroatoms independently selected from O, N and S; and wherein Ar is optionally substituted with 1 to 5 substituents independently selected from the group consisting of OH, Oxo (═O), halo, CN, NR′R″, C₁₋₄ alkyl, CF₃, C₁₋₄ haloalkyl, C₁₋₄ alkoxy, C₃₋₄ cycloalkyl, C₂₋₄ alkynyl; wherein R′ and R″ are independently selected from alkyl and hydrogen;
G is O or S;
each V is independently selected from methyl, cyanomethyl, or any two V combine to form a bridge or spirocycle structure optionally comprising a heteroatom in the bridge or spirocycle selected from S, SO₂, O or N, and wherein the bridge or spirocycle structure is optionally substituted with oxo
X is C—H, C-halo, C—C₁₋₃ alkyl, CF₃, C—C₁₋₃ haloalkyl, C—C₃₋₄ cycloalkyl, C-cyano, N;
Y is C—H, C-halo, C—C₁₋₃ alkyl, C—C₃₋₄ cycloalkyl, C-cyano, N;
Z is O, NR^{Z}, S, or absent; wherein R^{Z} is hydrogen or methyl;
Z′ is null, substituted or unsubstituted alkylene, substituted or unsubstituted heterocyclylalkylene, substituted or unsubstituted heterocyclyloxyalkylene, substituted or unsubstituted alkoxalkylene, substituted or unsubstituted heteroalkylene, substituted or unsubstituted arylalkylene, substituted or unsubstituted aryloxyalkylene, substituted or unsubstituted heteroarylalkylene, substituted or unsubstituted heteroaryloxyalkylene, substituted or unsubstituted cycloalkylalkylene, or a substituted or unsubstituted cycloalkyloxyalkylene; and
L is: bond, NH, S, O, C(O), C(O)O, OC(O), NHC(O), C(O)NH, NHC(O)NH, NHC(NH)NH, C(S), substituted or unsubstituted alkylene, substituted or unsubstituted heteroalkylene, substituted or unsubstituted cycloalkylene, substituted or unsubstituted spirocycloalkylene, substituted or unsubstituted heterocycloalkylene, substituted or unsubstituted spiroheterocycloalkylene, substituted or unsubstituted arylene, substituted or unsubstituted heteroarylene or combinations thereof.

In embodiments, G is O. In embodiments, G is S. In alternate embodiments, G is SOₚ, wherein p is 1 or 2.

In embodiments, Ar is selected from:

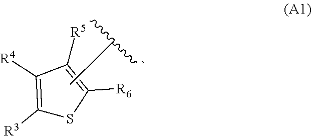

wherein R³, R⁴, R⁵, and R⁶ are independently selected from halogen, hydrogen, hydroxyl, alkoxy, alkyl, cycloalkyl, amino, N-alkylamino, C-amide (—CONRR′), N-amides (—NHCOR), urea (—NHCONHR), ether (—OR), sulfonamide (—NHSO₂R or —SO₂NHR), and CF₃; wherein each R and R′ is independently hydrogen, alkyl, or cycloalkyl; or any two adjacent R³, R⁴, R⁵, or R⁶ form an optionally substituted fused 5- or 6-membered ring comprising 0 to 3 heteroatoms selected from N, O or S; provided that one of R³, R⁴, R⁵, or R⁶ is the bond representing the link between A and the tricyclic ring system;

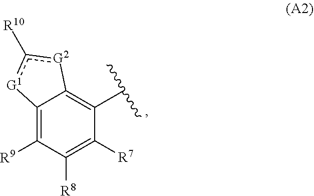

wherein G¹ and G² are independently selected from S, O, CH, —CH═CH—, —C-E, CR²⁰═CR²¹, wherein R²⁰ and R²¹ is hydrogen, halogen, alkyl or haloalkyl and at least one of R²⁰ and R²¹ is halogen, —CH═N—, —C-halogen, —C—OH, N, NH, and NMe or —CE, wherein E is CN; wherein either G¹ or G² forms a double bond with carbon of the —CR¹⁰ moiety; wherein R⁷, R⁸, and R⁹, are independently selected from halogen, hydrogen, hydroxyl, alkoxy, alkyl, cycloalkyl, amino, N-alkylamino; and R¹⁰ is hydrogen, hydroxyl, alkoxy, amino, N-alkylamino, C-amide (—CONRR′), N-amides (—NHCOR), urea (—NHCONHR), ether (—OR), or sulfonamide (—NHSO₂R or —SO₂NHR)₃; or (A2), wherein G¹ and G² are independently selected from S, O, CH, —CH═CH—, —CH═N—, N, NH, NMe or —CE, where E is CN, halogen, OH, OMe, alkyl- or aryl sulfonamide, alkyl- or aryl sulfone, acyl, formyl, amide, ester, carboxylic acid, or CF₃; wherein either G¹ or G² forms a double bond with carbon of the —CR¹⁰ moiety; wherein R⁷, R⁸, and R⁹, are independently selected from halogen, hydrogen, hydroxyl, alkoxy, alkyl, cycloalkyl, amino, N-alkylamino; and R¹⁰ is hydrogen, hydroxyl, alkoxy, amino, N-alkylamino, C-amide (—CONRR′), N-amides (—NHCOR), urea (—NHCONHR), ether (—OR), or sulfonamide (—NHSO₂R or —SO₂NHR)₃;

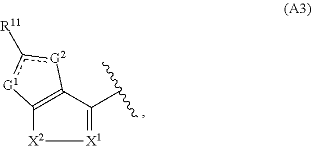

wherein G¹ and G² are independently selected from S, O, CH, —CH═CH—, —CH═N—, N, NH, and NMe; wherein either G¹ or G² forms a double bond with carbon of the —CR¹¹ moiety; R″ is hydroxyl, alkoxy, amino, N-alkylamino, C-amide (—CONRR′), N-amides (—NHCOR), urea (—NHCONHR), ether (—OR), or sulfonamide (—NHSO₂R or —SO₂NHR)₃, wherein R and R′ are independently alkyl or hydrogen; wherein X¹ is selected from CH, or N; and X² is selected from O, S, NH, and NMe; or (A3), wherein G¹ and G² are independently selected from S, O, CH, —CH═CH—, —CH═N—, N, NH, NMe or —CE, where E is CN, halogen, OH, OMe, alkyl- or aryl sulfonamide, alkyl- or aryl sulfone, acyl, formyl, amide, ester, carboxylic acid, or CF₃; wherein either G¹ or G² forms a double bond with carbon of the —CR¹¹ moiety; R¹¹ is hydroxyl, alkoxy, amino, N-alkylamino, C-amide (—CONRR′), N-amides (—NHCOR), urea (—NHCONHR), ether (—OR), or sulfonamide (—NHSO₂R or —SO₂NHR)₃, wherein R and R′ are independently alkyl or hydrogen; wherein X¹ is selected from CH, or N; and X² is selected from O, S, NH, and NMe;

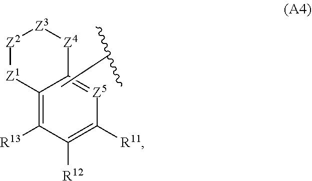

wherein Z¹, Z², Z³, and Z⁴ are independently selected from N, NH, CH, C-halo, C-alkynyl, C═O, C═S, point of attachment:

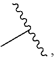

S, or null; Z⁵ is CH, C-halo, or point of attachment:

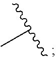

wherein null can only occur once, and at least one of Z¹, Z², Z³, and Z⁴ is NH and at least one of Z¹, Z², Z³, and Z⁴ adjacent to the NH is C═O or C═S; and wherein R^{D}, R¹², and R¹³, are independently selected from halogen, hydrogen, hydroxyl, alkoxy, alkyl, cycloalkyl, amino, N-alkylamino; or

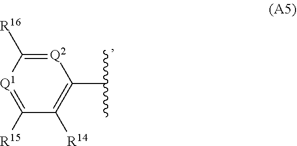

wherein Q¹ and Q² are independently selected from N, —CH, C-halogen, with the proviso that at least one of Q¹ and Q2 is N; R¹⁴ and R¹⁵ are selected from halogen, hydrogen, hydroxyl, alkoxy, alkyl, CF₃ haloalkyl, cycloalkyl, amino, N-alkylamino; and R¹⁶ is hydroxyl, alkoxy, amino, N-alkylamino, C-amide (—CONRR′), N-amide (—NHCOR), urea (—NHCONHR), ether (—OR), or sulfonamide (—NHSO₂R or —SO₂NHR)₃, wherein R and R′ are independently alkyl or hydrogen.

In embodiments, (A1) is selected from:

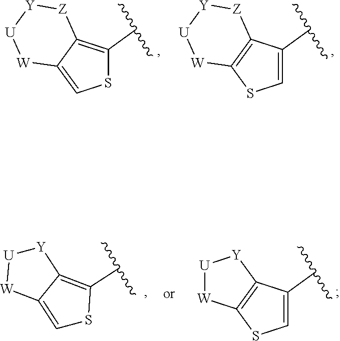

wherein each W, U, Y, and Z are independently selected from C═O, NH, O, S, N, CH, C-Q, where Q is amino, alkylamino, OH, halogen, methyl, —O-alkyl, —O-cycloalkyl, trifluoromethyl, amide, and urea.

In embodiments, (A1) is selected from:

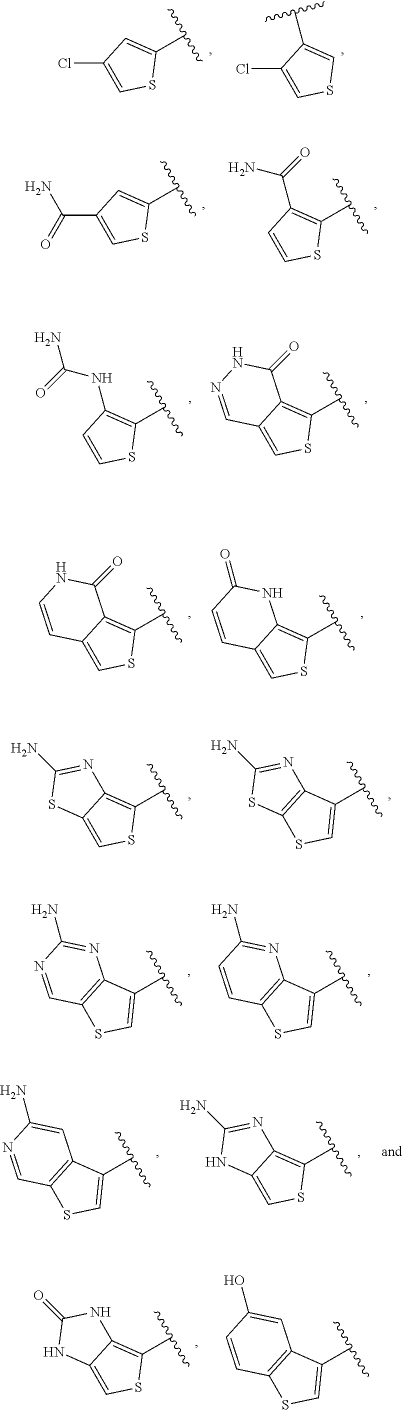

In embodiments, (A2) is selected from:

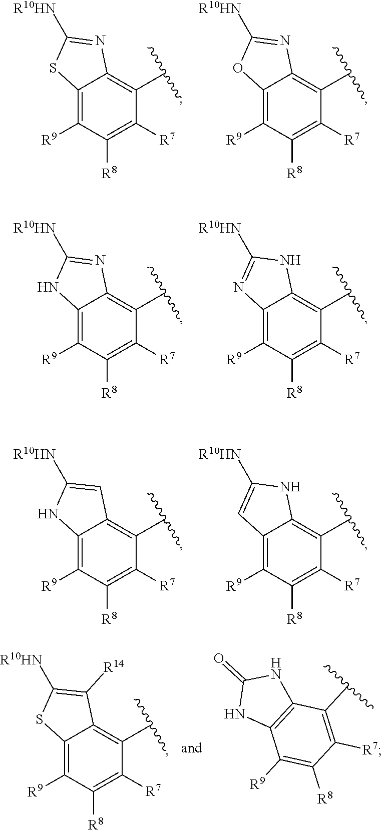

wherein R¹⁴ is CN.

In embodiments, (A2) is:

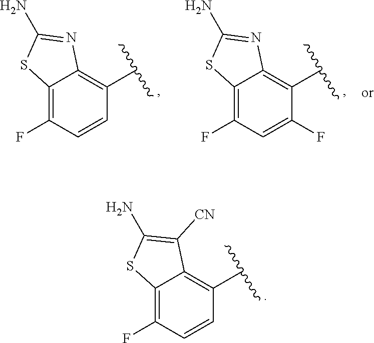 [SMILES: *c1ccc(F)c2sc(N)nc12 ; *c1c(F)cc(F)c2sc(N)nc12 ; *c1ccc(F)c2sc(N)c(C#N)c12]

In embodiments, (A3) is selected from:

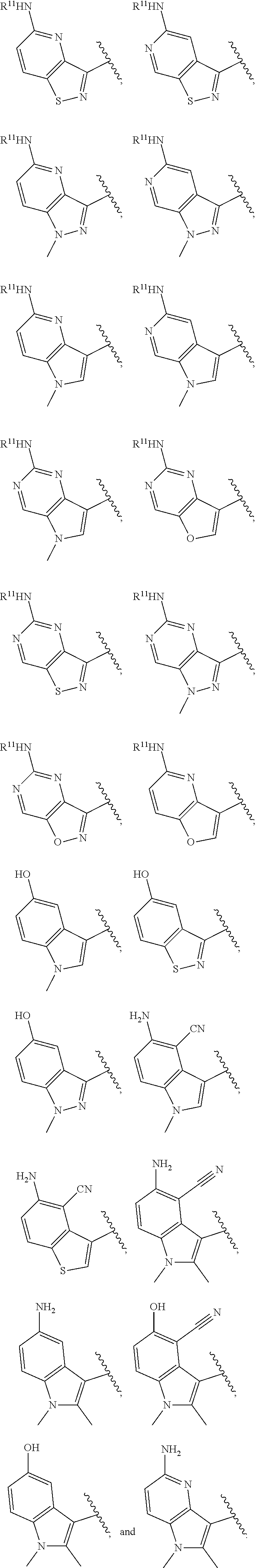

In embodiments, (A4) is selected from:

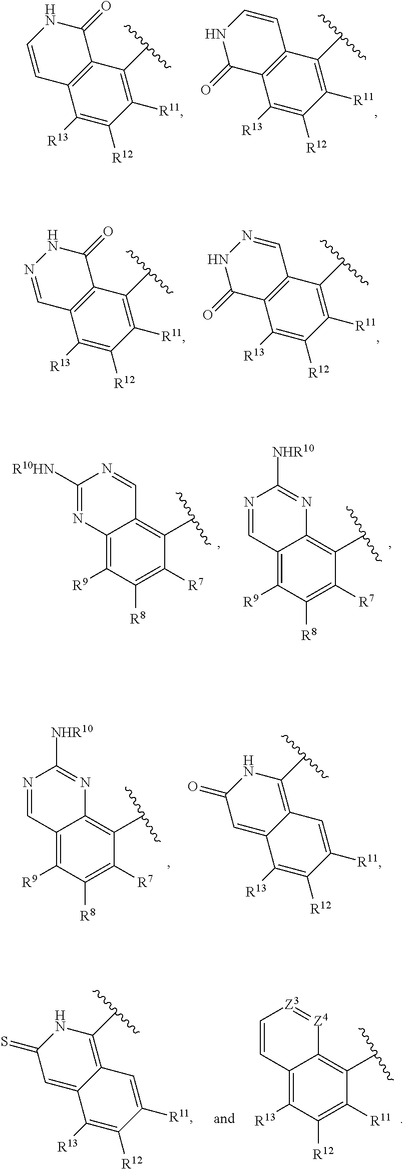

In embodiments, (A4) is selected from:

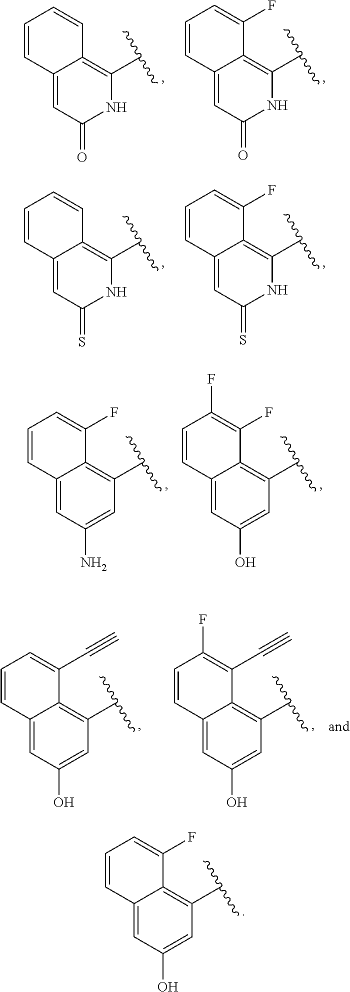

In embodiments, (A5) is

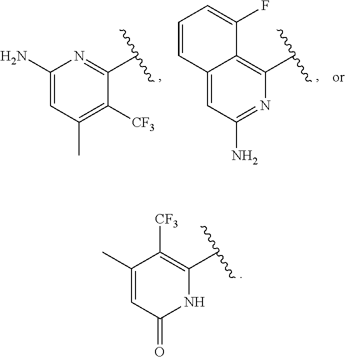

The linking piperazine unit in Formulas (I) and (II) can comprise any array of substitutions. In embodiments, two V form a bridge: —CH₂—CH₂—. In some embodiments, the linking piperazine can be selected from the following structures:

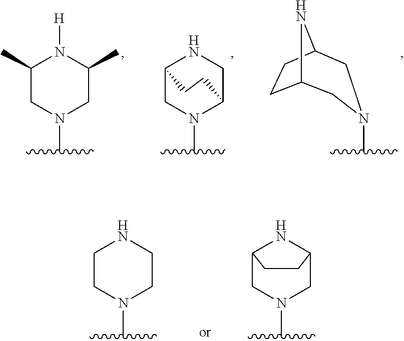

In embodiments, G12D- in Formula (A) is selected from:

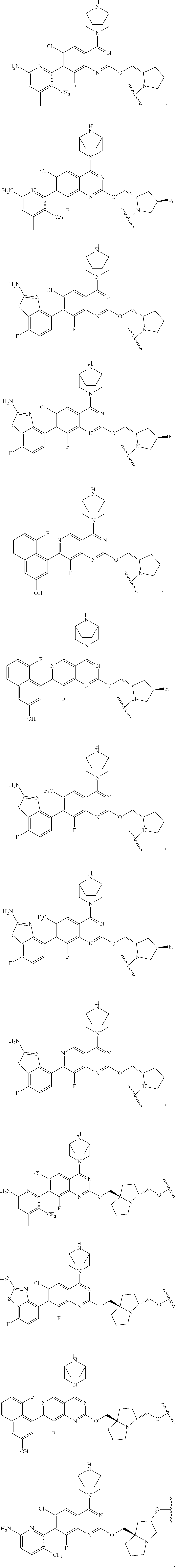

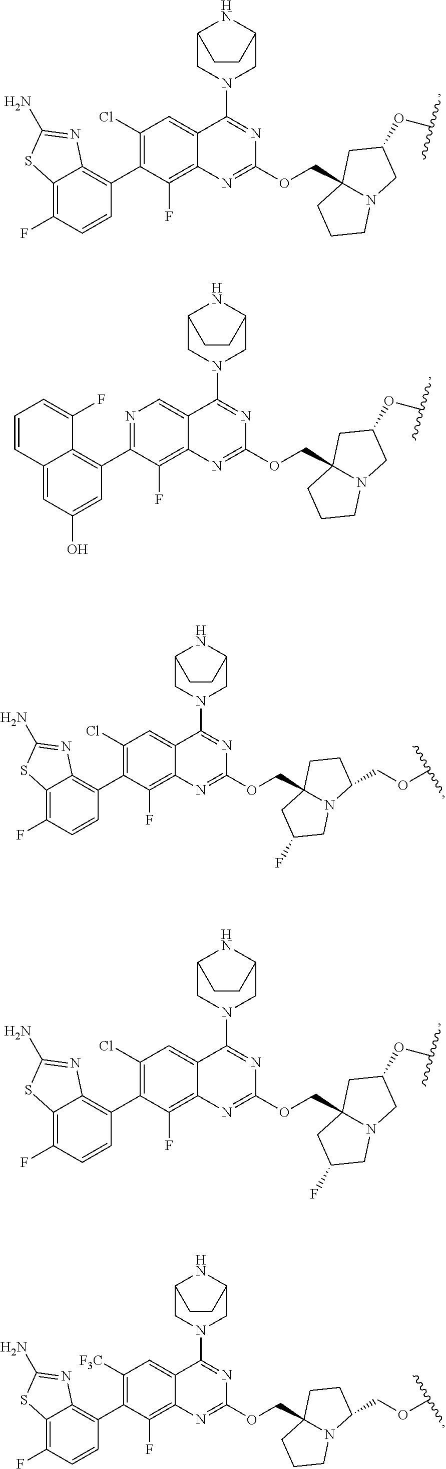

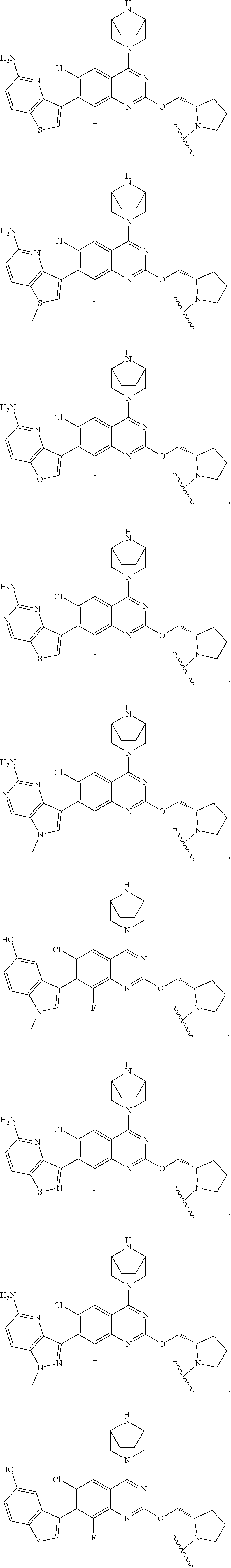

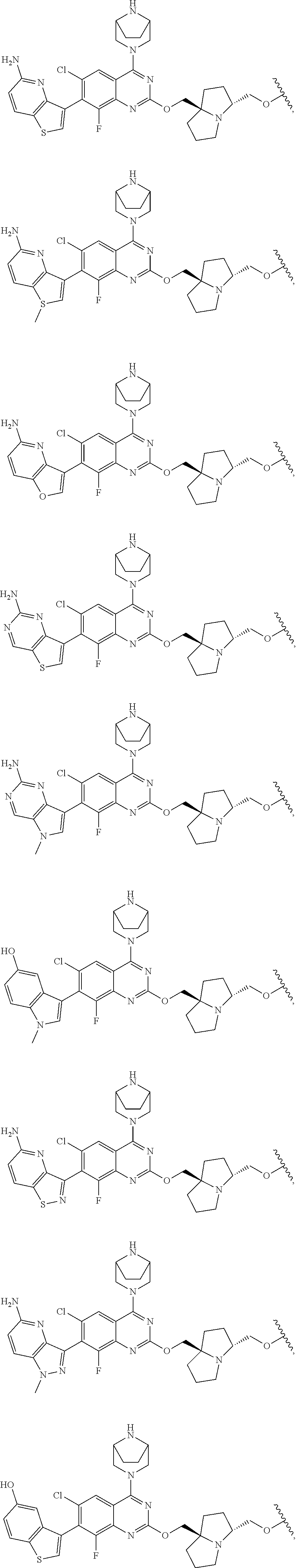

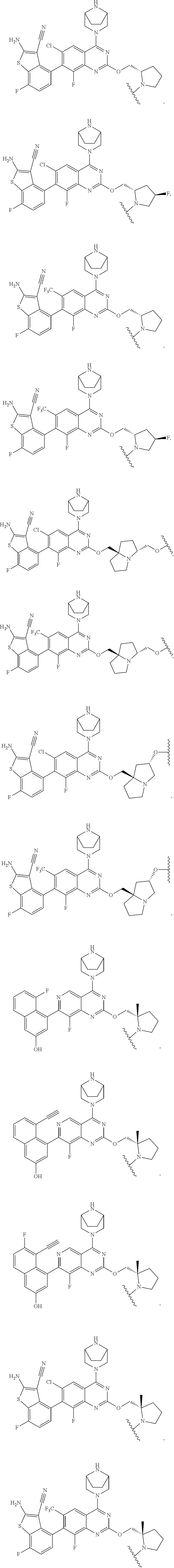

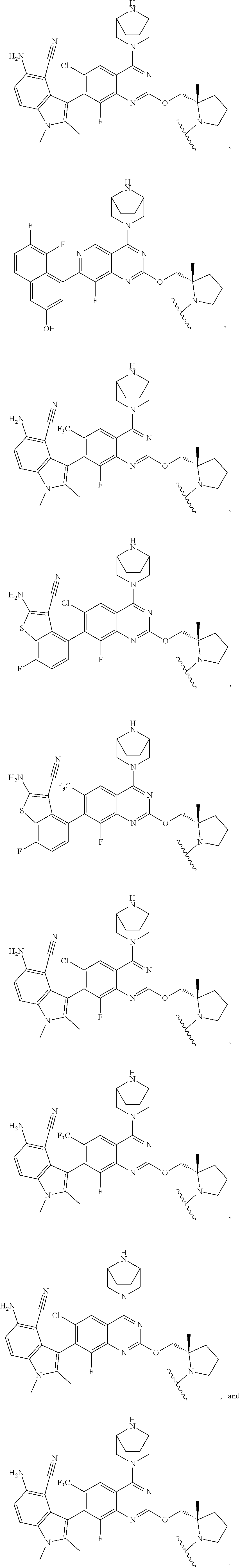

In embodiments, G12D- in Formula (A) is selected from:

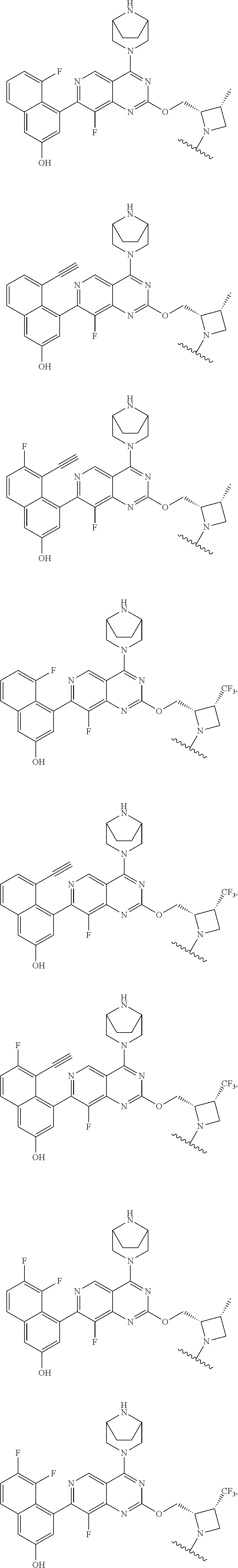

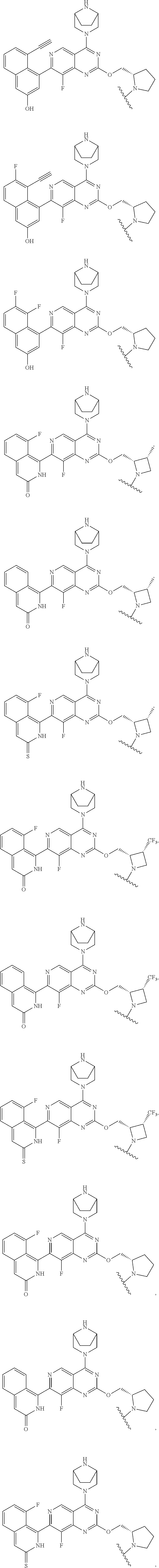

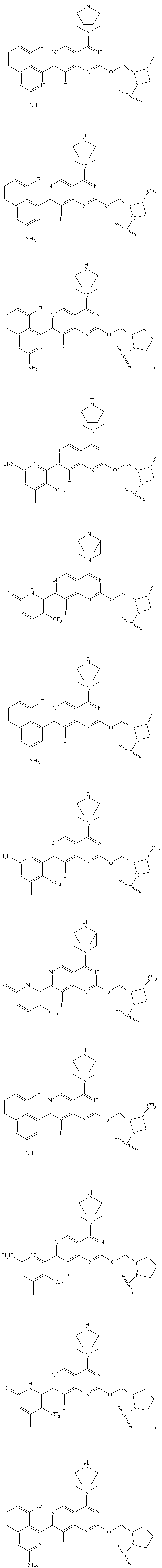

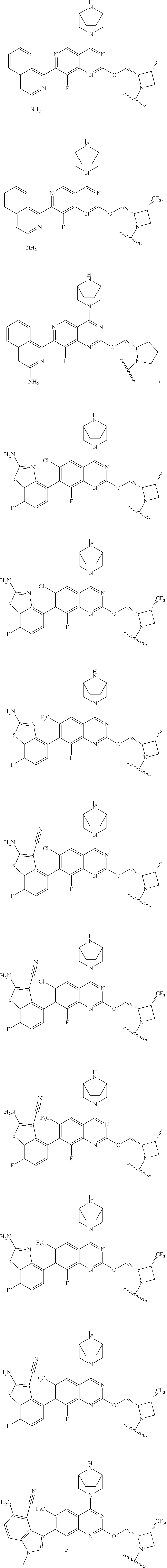

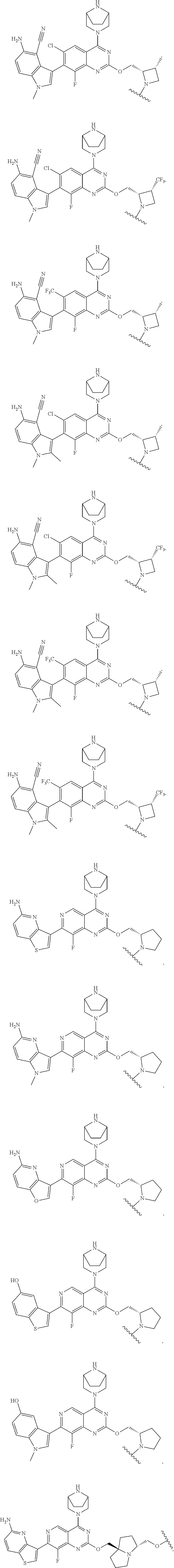

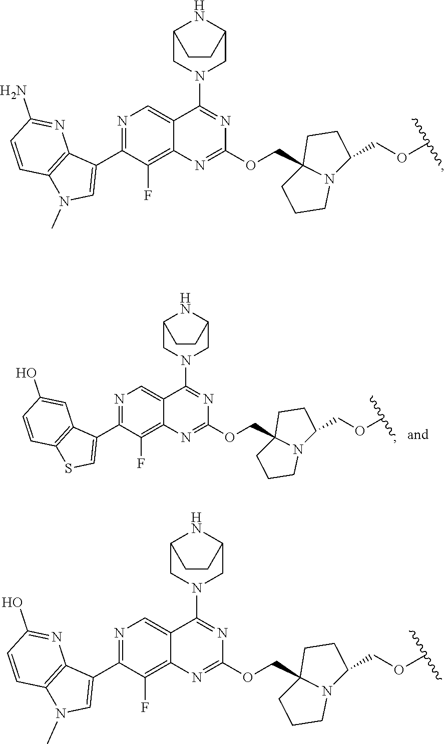

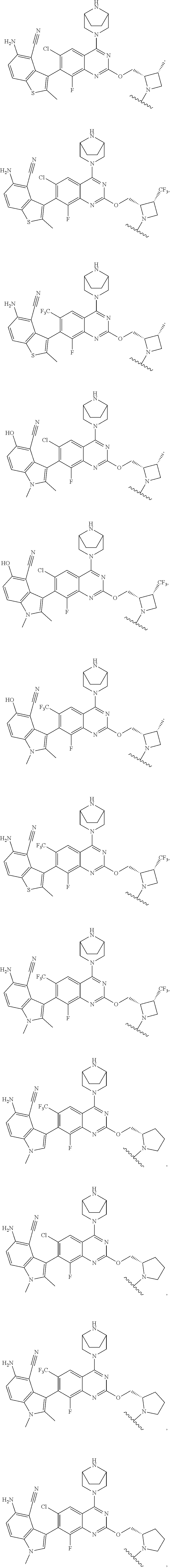

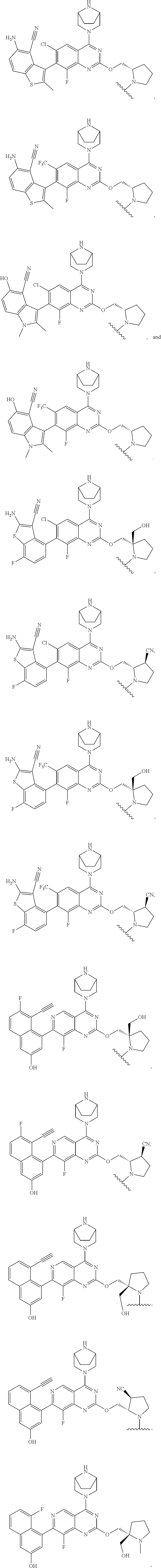

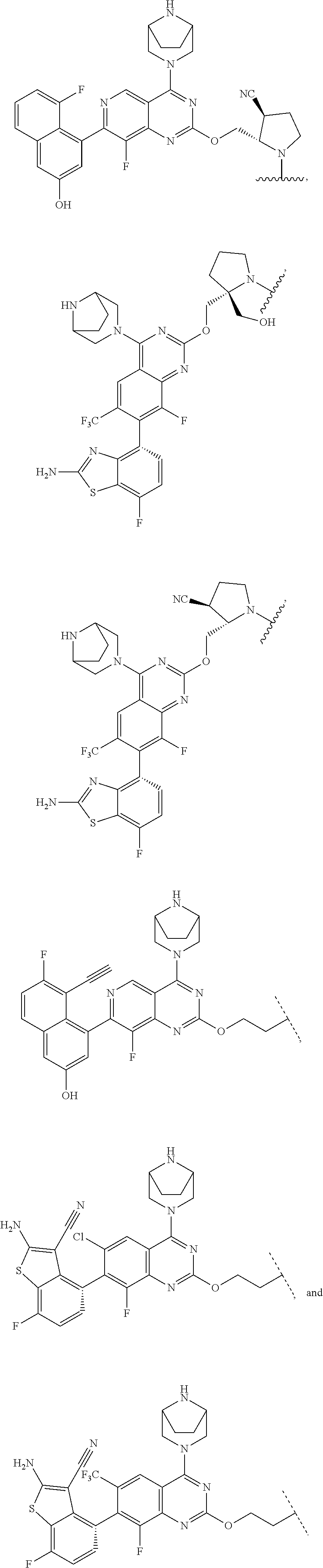

In embodiments, linker L can be assembled from any combination of the following elements: bond, NH, S, O, C(O), C(O)O, OC(O), NHC(O), C(O)NH, NHC(O)NH, NHC(NH)NH, C(S), substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene. Examples of linkers include straight chain alkylenes, polyethylene glycols, polypropylenglycols, and the like. In embodiments, linker -L- is selected from:

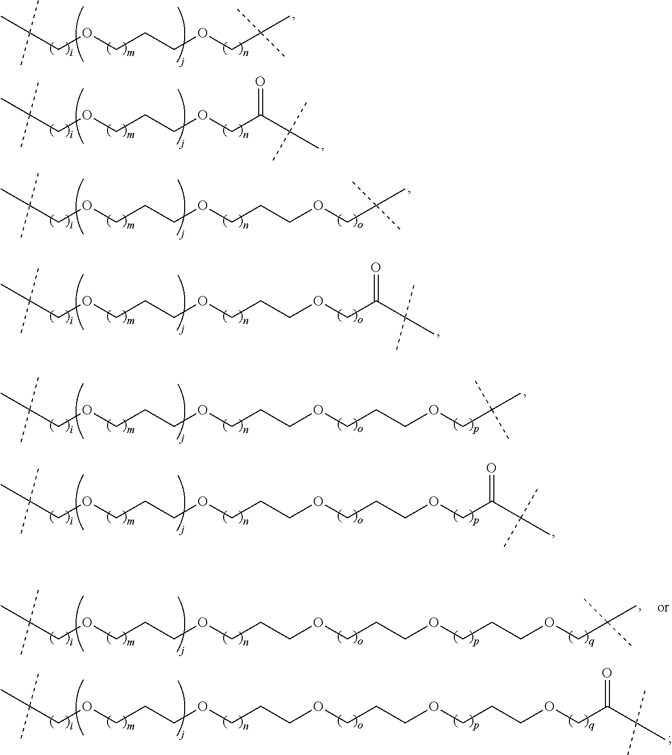

wherein any number of carbon or oxygen atoms of L are optionally exchanged with a heteroatom selected from NR^{L} S, or SO₂ atoms, where R^{L} is hydrogen or methyl; and i, j, m, n, n, o, p, q are integers independently selected from 0, 1, 2, 3, 4, 5, 6, 7, 8, 9 and 10; and wherein each carbon atom of L is optionally substituted with oxo (═O), OH, halo, NR₃R₄, C₁₋₄ alkyl, C₁₋₄ haloalkyl, C₁₋₄ alkoxy, C₃₋₄ cycloalkyl; wherein each L is attached to Z′ and ubiquitin binding moiety (UBM) in either orientation.

In embodiments, linker -L- is selected from:

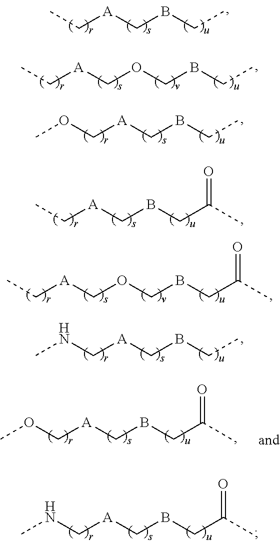

wherein each A and B are independently selected from absent, a 3 to 10 membered cycloalkylene, 4- to 10-membered heterocycloalkylene, 5- to 10-membered heteroarylene and 6- to 10-membered arylene, wherein any number of carbon or oxygen atoms of L are optionally exchanged with a heteroatom selected from NR^{L}, S or SO₂ atoms, where R^{L} is hydrogen or methyl; r, s, u, v are integers independently selected from 0, 1, 2, 3, 4, 5, 6, 7, 8, 9 and 10; wherein each carbon atom of L is optionally substituted with oxo (═O), OH, halo, NR₃R₄, C₁₋₄ alkyl, C₁₋₄ haloalkyl, C₁₋₄ alkoxy, C₃₋₄ cycloalkyl; wherein each L is attached to Z′ and ubiquitin binding moiety (UBM) in either orientation; and when A and B are a 3- to 10-membered cycloalkyl or 4- to 10-membered heterocycloalkyl, they are incorporated at any two independent ring atoms, or at the same ring atom.

In embodiments, L is selected from:

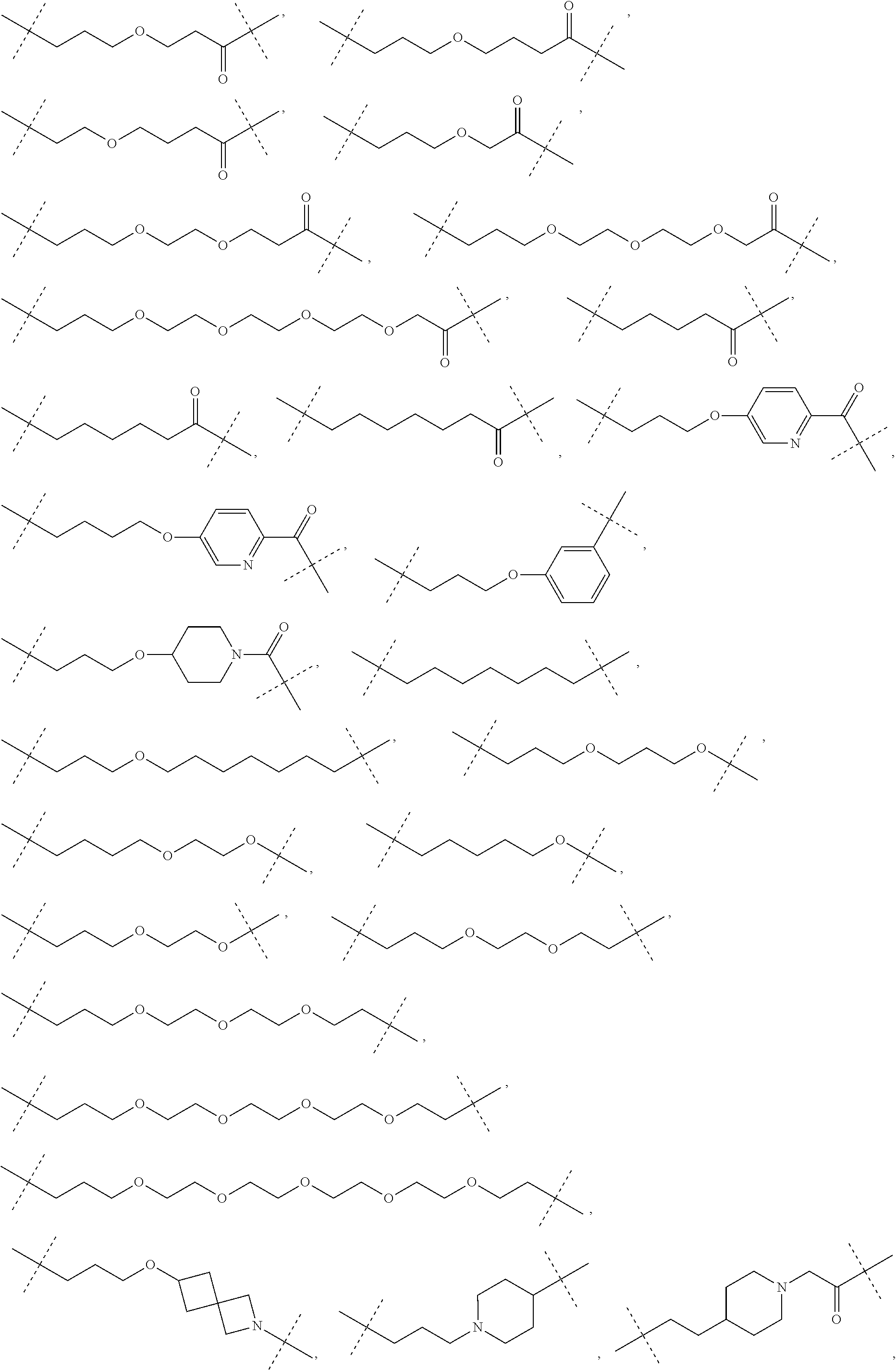

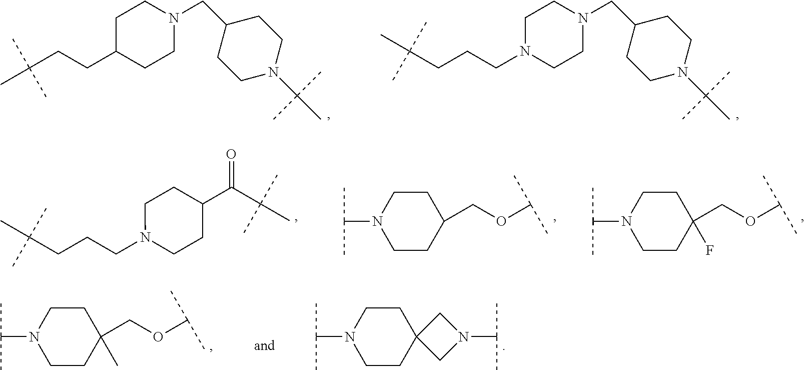

In embodiments, L is a structure according to formula (L1) or (L2):

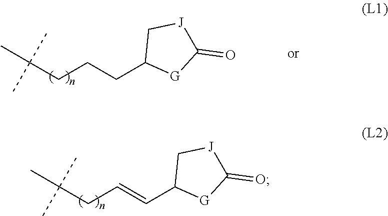

J is null, —CH₂—, O, —OCH₂—, —CH₂O—, —CH₂CH₂—;
G is a nitrogen attached to a ubitquitin binding moiety (UBM) ligand; and
n is an integer selected from 0, 1, and 2.

In embodiments, the structures according to (L1) or (L2) are bonded to a nitrogen that belongs to a VHL ligand to form a tertiary amine. For example, G is equivalent to the nitrogen that forms the lactam in the following structure that bonds the linker to the VHL:

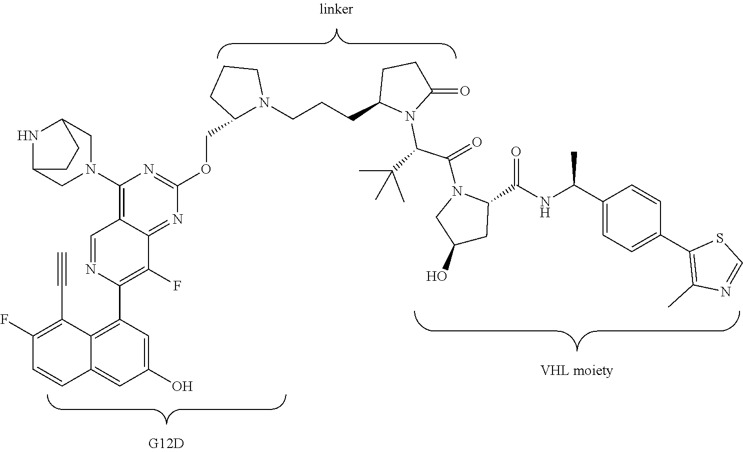 [SMILES: C#Cc1c(F)ccc2cc(O)cc(-c3ncc4c(N5CC6CCC(C5)N6)nc(OC[C@@H]5CCCN5CCC[C@H]5CCC(=O)N5[C@H](C(=O)N5C[C@H](O)C[C@H]5C(=O)N[C@@H](C)c5ccc(-c6scnc6C)cc5)C(C)(C)C)nc4c3F)c12]

In embodiments, L is:

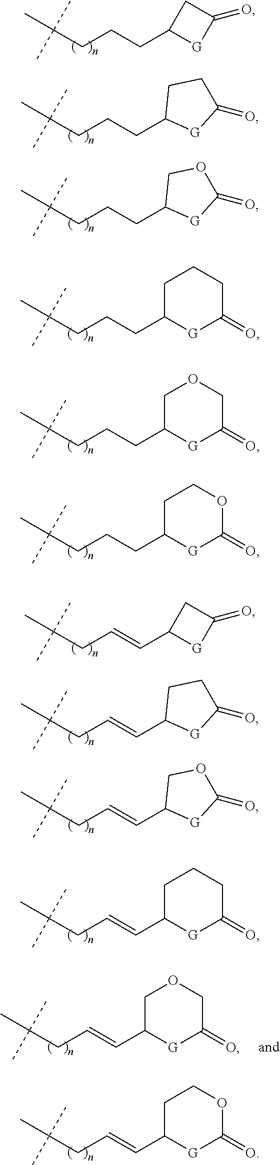

In embodiments, L is a formula according to (L1) or (L2) and n is 1.

In embodiments, the UBM is a von Hippel-Lindau (VHL) or cereblon ligand. In embodiments, UBM is a group (derivatized or configured to be linked or coupled to an G12D inhibitor via a linker (as indicated by the dashed line) according to Formula (III):

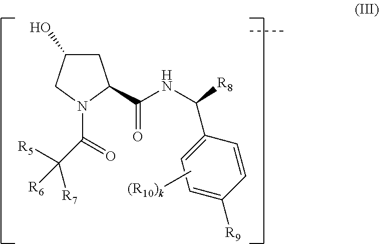

R₅ and R₆ are independently hydrogen, optionally substituted alkyl, optionally substituted cycloalkyl, optionally substituted hydroxyalkyl, optionally substituted heteroaryl, or haloalkyl; or R₅, R₆, and the carbon atom to which they are attached form an optionally substituted cycloalkyl; R₇ is optionally substituted heterocyclic, optionally substituted alkoxy, optionally substituted heteroaryl, optionally substituted aryl,

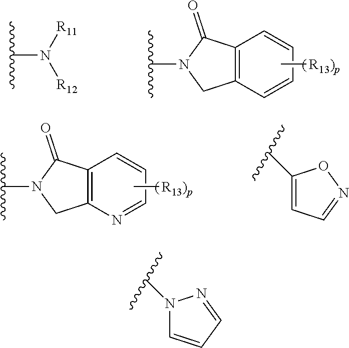

wherein,
R₈ is H, hydroxyalkyl, haloalkyl, or optionally substituted alkyl;
R₉ is selected from the group consisting of H, halogen, CN, OH, optionally substituted heteroaryl, optionally substituted aryl;
R₁₀ is H, OH, halogen, optionally substituted alkyl, optionally substituted alkoxy; k=0, 1, 2, 3, 4;
R₁₁ is H or optionally substituted alkyl;
R₁₂ is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
each R₁₃ is independently halo, optionally substituted alkoxy, cyano, optionally substituted alkyl, haloalkyl, haloalkoxy or a linker, p is an integer from 0 to 4.

In certain additional embodiments, R₉ of Formula (III) is selected from the group consisting of:

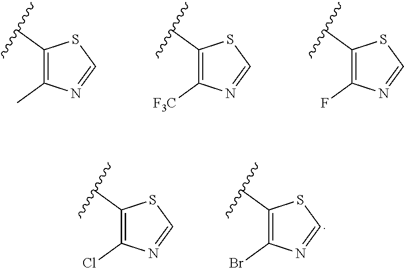

In certain embodiments, UBM has a chemical structure selected from the group of Formulas (IVa)-(IVd):

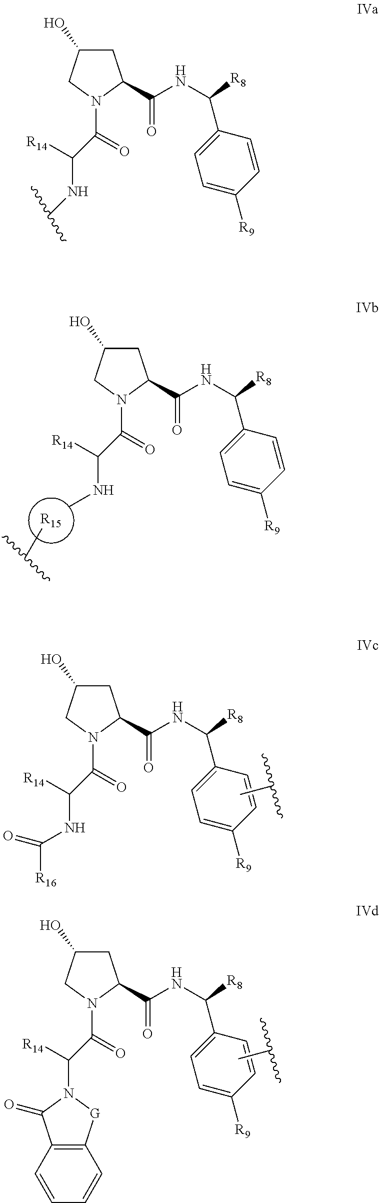

wherein R₈ is H, haloalkyl, or optionally substituted alkyl; R₉ is selected from the group consisting of H, halogen, CN, OH, optionally substituted heteroaryl, optionally substituted aryl; R₁₄ is H, haloalkyl, or optionally substituted alkyl (including tert-butyl, iso-propyl); R₁₅ is an optionally substituted 5 or 6 membered heteroaryl or optionally substituted aryl; R₁₆ is optionally substituted alkyl, optionally substituted cycloalkyl; and G is CH₂ or C═O.

In embodiments, R₉ in Formulas (IVa)-(IVd) is selected from the group consisting of:

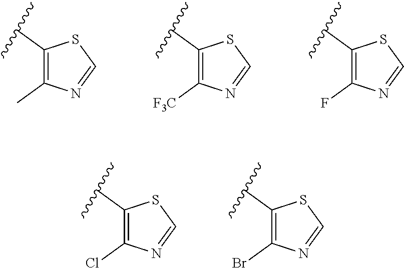

In embodiments, the VHL ligand is selected from:

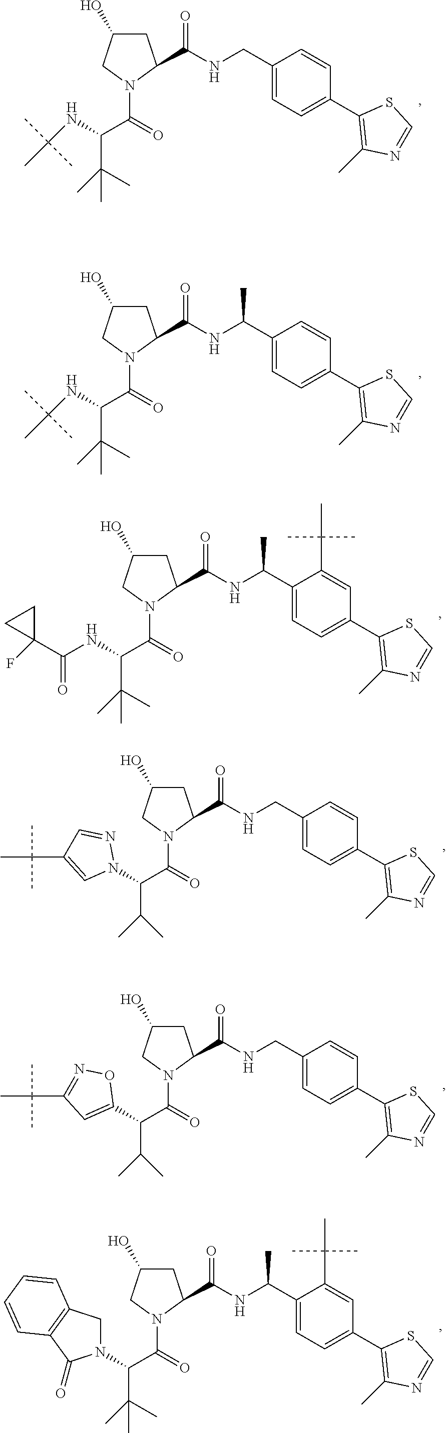

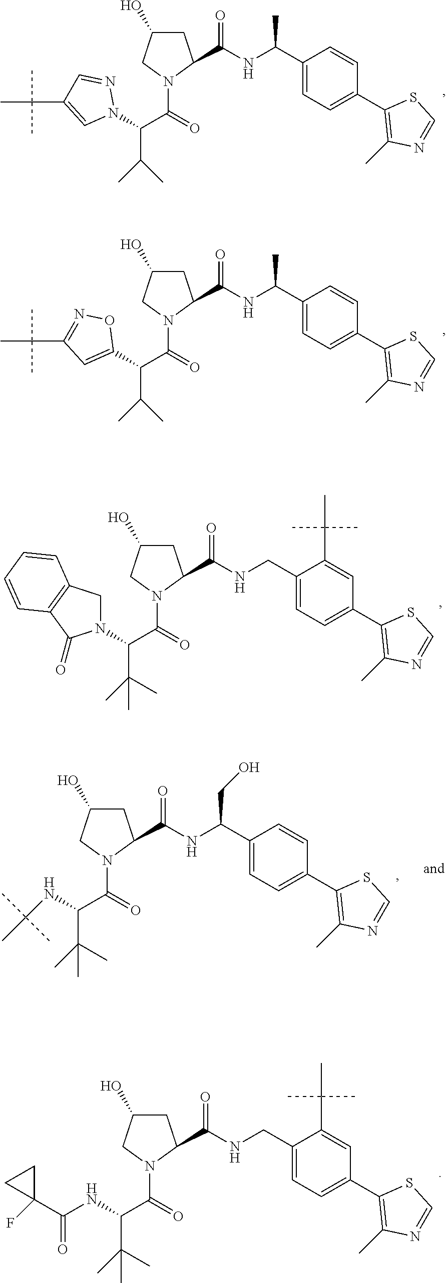

In embodiments, the VHL ligand is selected from:

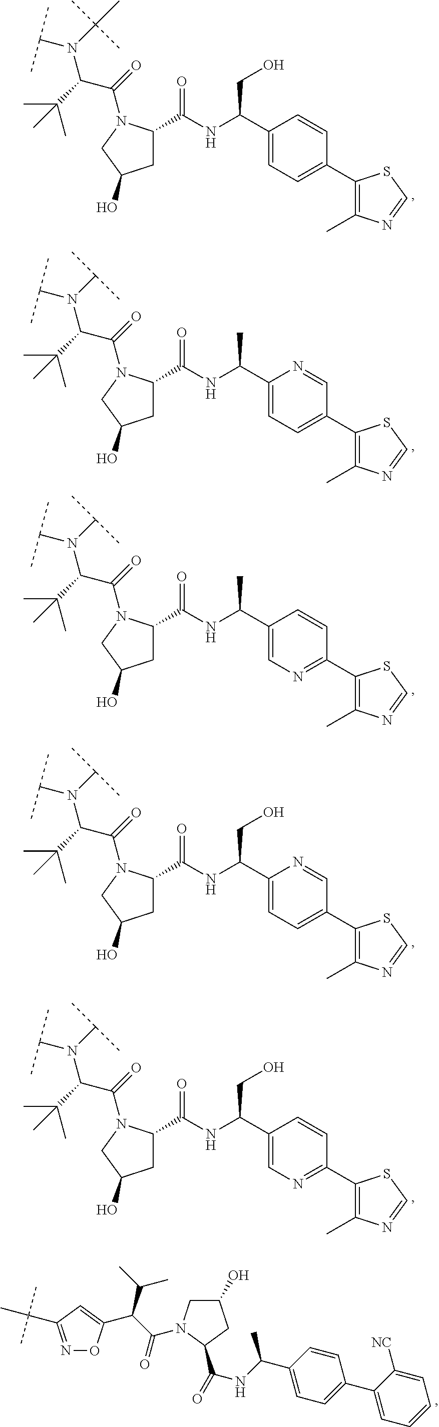

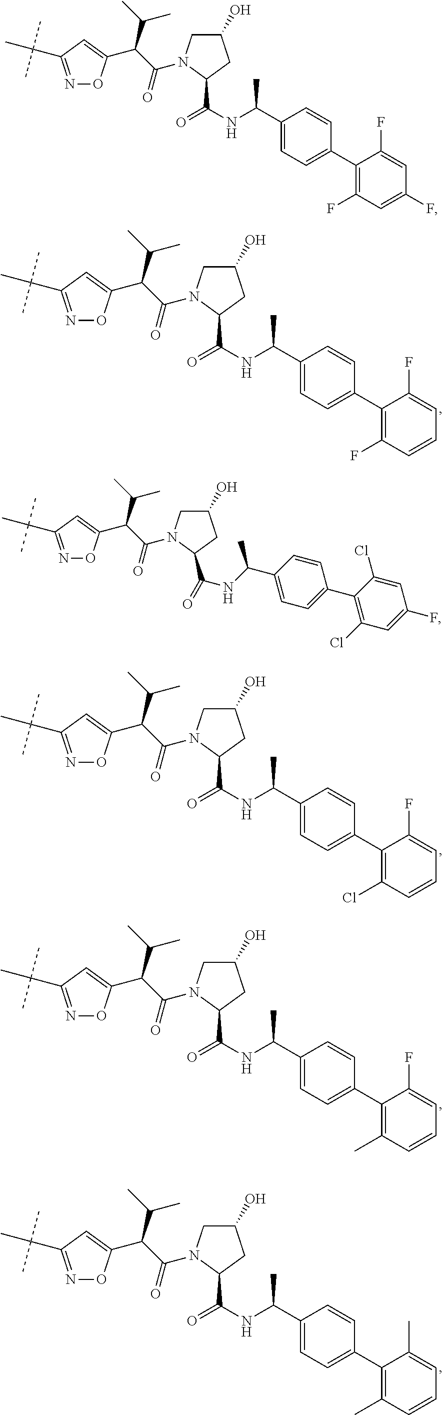

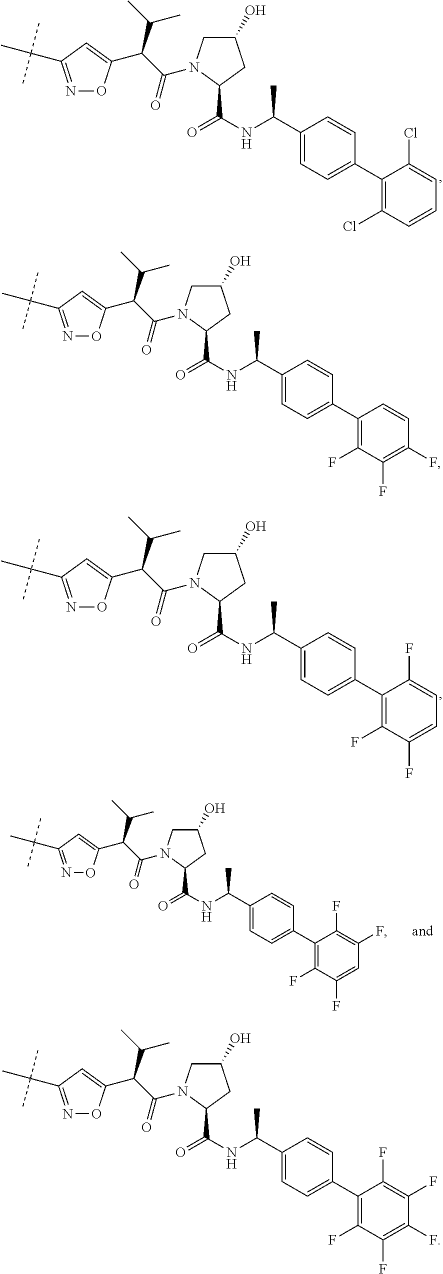

In embodiments, the cereblon ligand is selected from Formulas (Va)-(Vf):

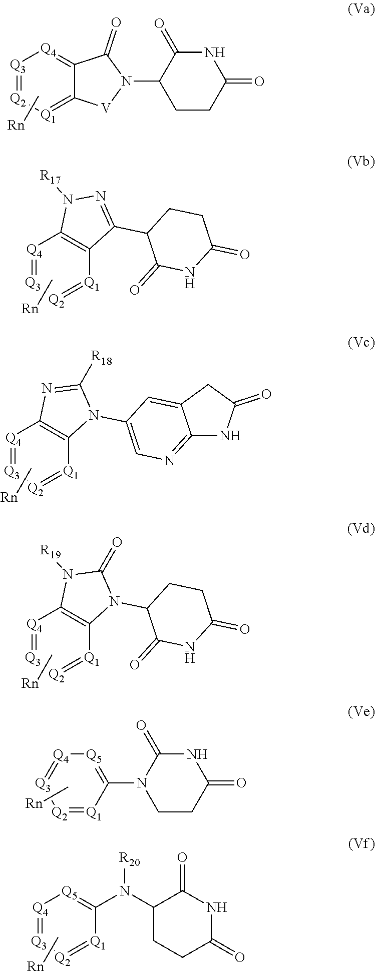

wherein V is selected from the group CH₂, C═O, SO₂; Q₁-Q₄ are independently N or a carbon C substituted with a group independently selected from H, R; n is an integer from 1-4; R₁₇ is H, optionally substituted alkyl; R₁₈ is H, optionally substituted alkyl; R₁₉ is H, optionally substituted alkyl; and R₂₀ is H or optionally substituted alkyl.

In embodiments, R of Formulas (Va) through (Vf) is selected from: H, OH, halo, CN, —C(═O)R′ (e.g., a carboxy group), —CONR′R″ (e.g., an amide group), —OR′ (e.g., OH), —NR′R″ (e.g., an amine group), —SR′, —SO₂R′, —SO₂NR′R″, —CR′R″—, —CR′NR′R″—, optionally substituted linear or branched alkyl, optionally substituted hydroxualkyl, optionally substituted cycloalkyl, optionally substituted heterocyclyl, optionally substituted aryl, optionally substituted heteroaryl, optionally substituted alkoxyl group, optionally substituted alkyl-aryl, optionally substituted alkyl-cycloalkyl, optionally substituted alkyl-heterocyclyl, optionally substituted alkyl-heteroaryl, optionally substituted aryl-alkyl, optionally substituted cycloalkyl-alkyl, optionally substituted heterocyclyl-alkyl, optionally substituted heteroaryl-alkyl,

optionally substituted

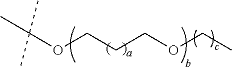

(e.g., optionally substituted with one or more halogen, alkyl, haloalky, cycloalkyl (e.g., a C₃-C₆ cycloalkyl), or optionally substituted aryl),
optionally substituted

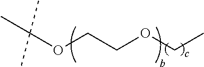

(e.g., optionally substituted with one or more halogen, alkyl, haloalky, cycloalkyl (e.g., a C3-C6 cycloalkyl), or optionally aryl), CCR′, CR′═CR′R″; each of a, b, and c are independently 0, 1, 2, 3, 4, 5, or 6; R′ and R″ of Formulas (a) through (f) are independently selected from a bond, H, optionally substituted linear or branched alkyl, optionally substituted hydroxualkyl, optionally substituted cycloalkyl, optionally substituted aryl, optionally substituted heteroaryl, optionally substituted heterocyclyl, optionally substituted alkyl-aryl, optionally substituted alkyl-cycloalkyl, optionally substituted alkyl-heterocyclyl, optionally substituted alkyl-heteroaryl, optionally substituted aryl-alkyl, optionally substituted cycloalkyl-alkyl, optionally substituted heterocyclyl-alkyl, optionally substituted heteroaryl-alkyl.

The above stated R, R′ and R″ groups in Formulas (Va)-(Vf) can be further optionally substituted with OH, halo, —C(═O)R′ (e.g., a carboxy group), hydroxyalkyl, linear or branched alkyl, optionally substituted cycloalkyl, optionally substituted aryl, optionally substituted heteroaryl, optionally substituted heterocyclyl, optionally substituted alkyl-aryl, optionally substituted alkyl-cycloalkyl, optionally substituted alkyl-heterocyclyl, optionally substituted alkyl-heteroaryl; optionally substituted aryl-alkyl, optionally substituted cycloalkyl-alkyl, optionally substituted heterocyclyl-alkyl, optionally substituted heteroaryl-alkyl.

In embodiments, the UBM comprises a compound of Formula (VI):

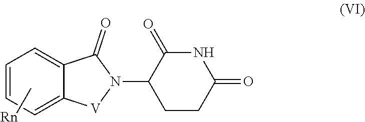

wherein V is selected from the group CH₂, C═O;
n is an integer from 1 to 4;
R is independently selected from H, OH, halo, CN, —C(═O)R′ (e.g., a carboxy group), —CONR′R″ (e.g., an amide group), —OR′ (e.g., OH), —NR′R″ (e.g., an amine group), —SR′, —SO₂R′, —SO₂NR′R″, —CR′R″—, —CR′NR′R″—, optionally substituted linear or branched alkyl, optionally substituted hydroxualkyl, optionally substituted cycloalkyl, optionally substituted heterocyclyl, optionally substituted aryl, optionally substituted heteroaryl, optionally substituted alkoxyl group, optionally substituted alkyl-aryl, optionally substituted alkyl-cycloalkyl, optionally substituted alkyl-heterocyclyl, optionally substituted alkyl-heteroaryl, optionally substituted aryl-alkyl, optionally substituted aryl-alkoxy, optionally substituted cycloalkyl-alkyl, optionally substituted heterocyclyl-alkyl, optionally substituted heteroaryl-alkyl, substituted heteroaryl-alkoxy, CCR′, CR′═CR′R″; R′ and R″ are independently selected from a bond, H, optionally substituted linear or branched alkyl, optionally substituted hydroxualkyl, optionally substituted cycloalkyl, optionally substituted aryl, optionally substituted heteroaryl, optionally substituted heterocyclyl, optionally substituted alkyl-aryl, optionally substituted alkyl-cycloalkyl, optionally substituted alkyl-heterocyclyl, optionally substituted alkyl-heteroaryl, optionally substituted aryl-alkyl, optionally substituted cycloalkyl-alkyl, optionally substituted heterocyclyl-alkyl, optionally substituted heteroaryl-alkyl;
The above stated R, R′ and R″ groups in Formula (VI) can be further optionally substituted with OH, halo, —C(═O)R′ (e.g., a carboxy group), hydroxyalkyl, linear or branched alkyl, optionally substituted cycloalkyl, optionally substituted aryl, optionally substituted heteroaryl, optionally substituted heterocyclyl, optionally substituted alkyl-aryl, optionally substituted alkyl-cycloalkyl, optionally substituted alkyl-heterocyclyl, optionally substituted alkyl-heteroaryl; optionally substituted aryl-alkyl, optionally substituted cycloalkyl-alkyl, optionally substituted heterocyclyl-alkyl, and optionally substituted heteroaryl-alkyl.

In embodiments, the cereblon ligand is selected from:

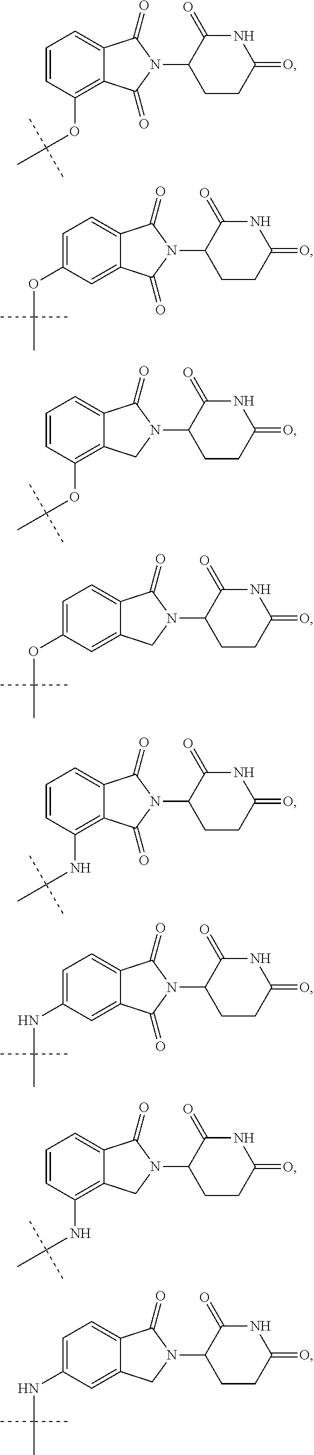

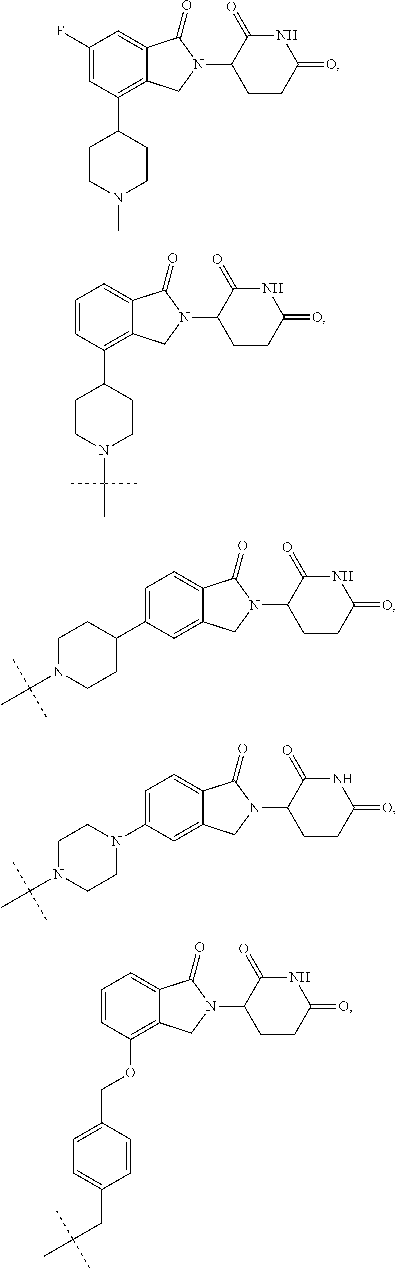

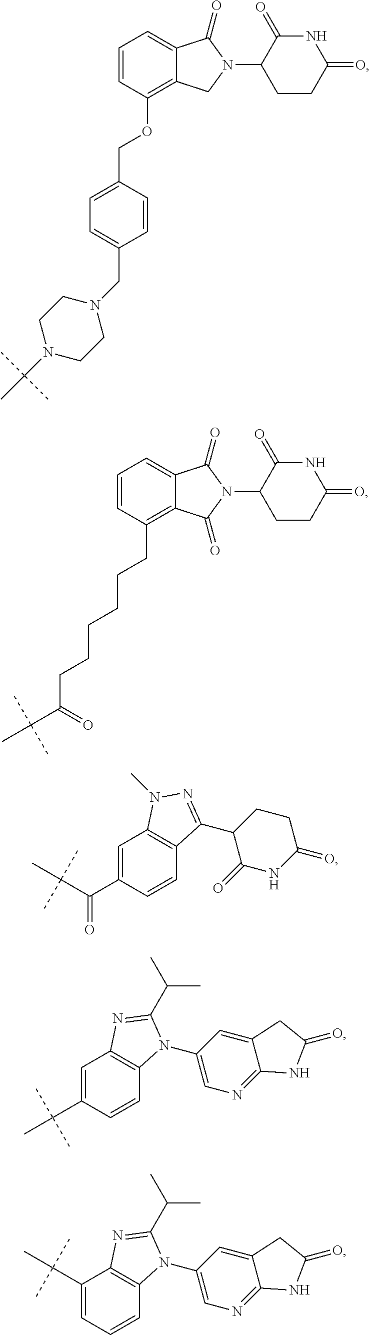

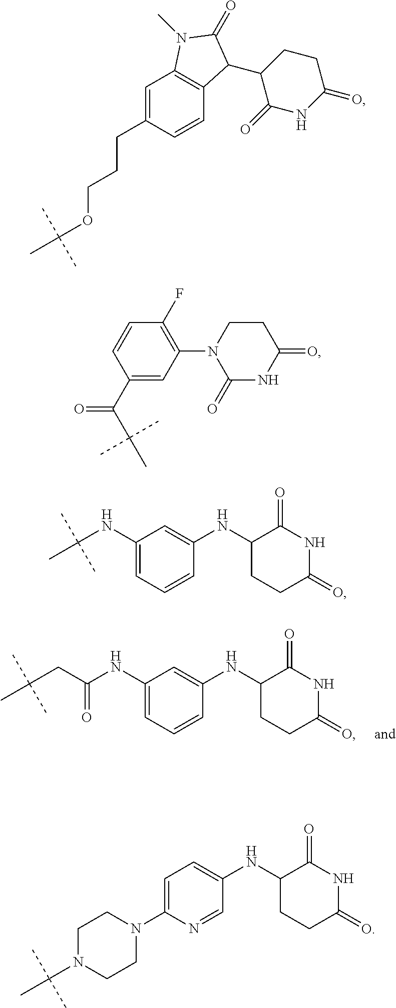

In some embodiments, the conjugate is selected from:

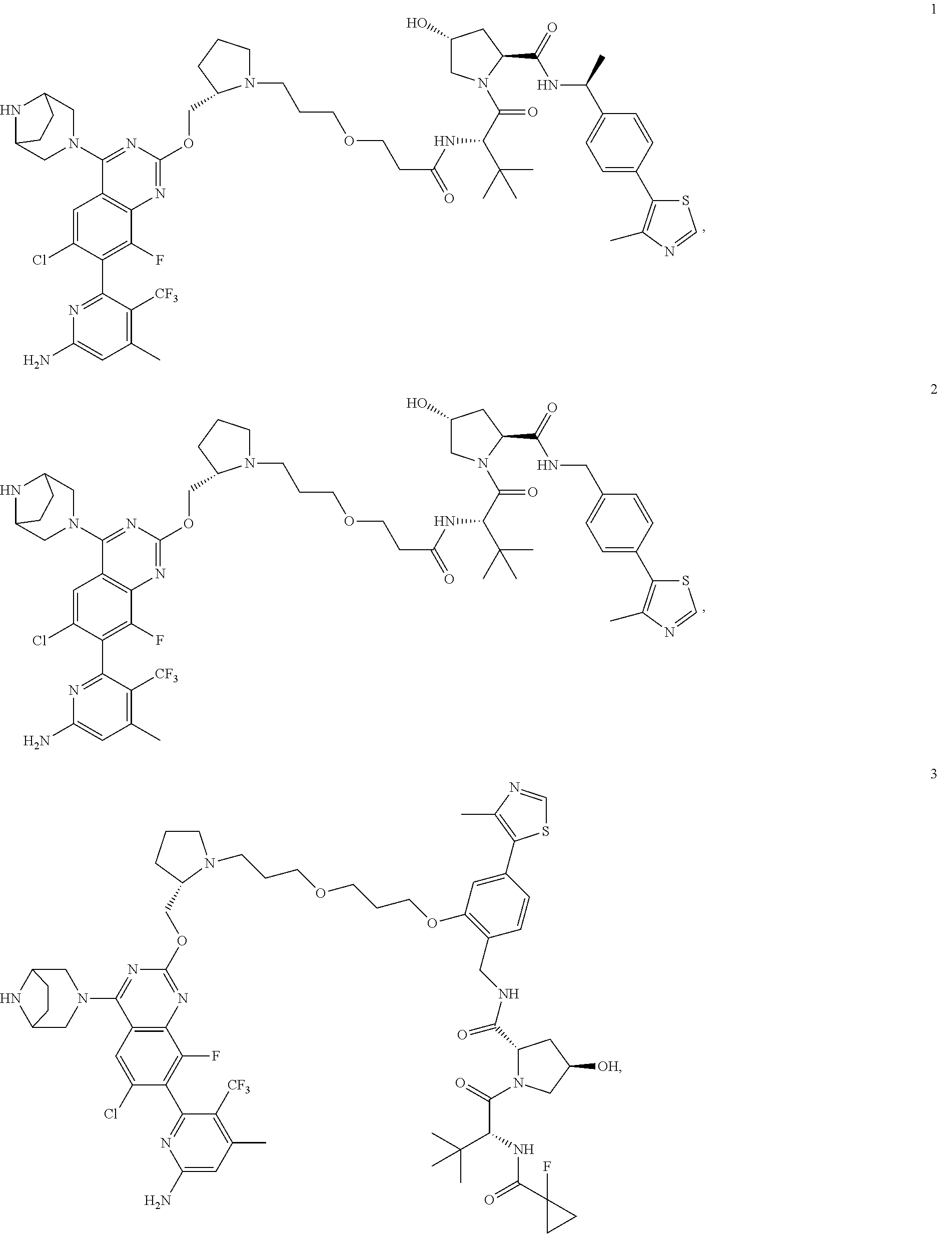 [SMILES: Cc1cc(N)nc(-c2c(Cl)cc3c(N4CC5CCC(C4)N5)nc(OC[C@@H]4CCCN4CCCOCCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F ; Cc1cc(N)nc(-c2c(Cl)cc3c(N4CC5CCC(C4)N5)nc(OC[C@@H]4CCCN4CCCOCCC(=O)N[C@H](C(=O)N4C[C@H](O)C[C@H]4C(=O)NCc4ccc(-c5scnc5C)cc4)C(C)(C)C)nc3c2F)c1C(F)(F)F ; Cc1cc(N)nc(-c2c(Cl)cc3c(N4CC5CCC(C4)N5)nc(OC[C@@H]4CCCN4CCCOCCCOc4cc(-c5scnc5C)ccc4CNC(=O)[C@@H]4C[C@@H](O)CN4C(=O)[C@H](NC(=O)C4(F)CC4)C(C)(C)C)nc3c2F)c1C(F)(F)F]

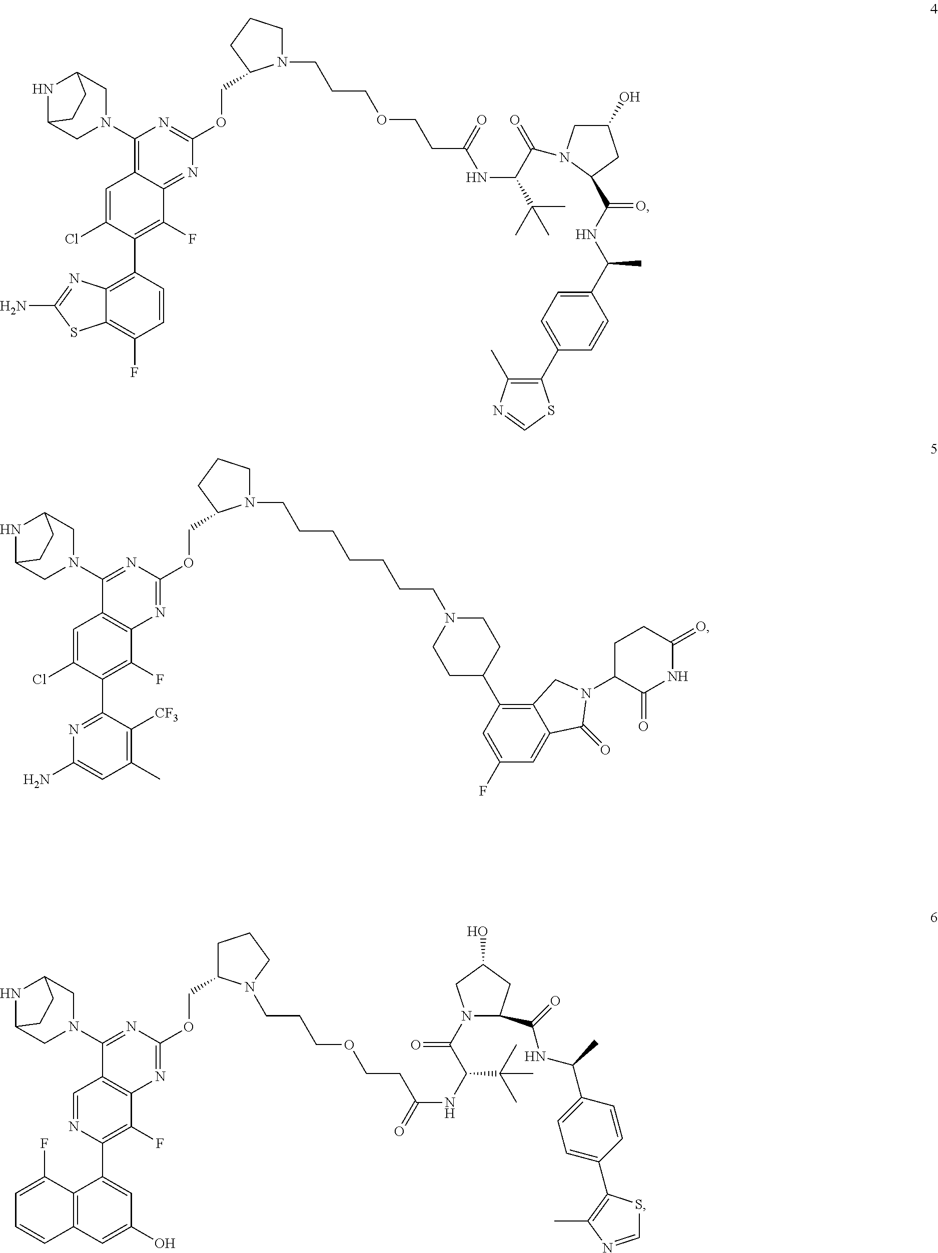 [SMILES: Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cc(Cl)c(-c4ccc(F)c5sc(N)nc45)c(F)c3n2)C(C)(C)C)cc1 ; Cc1cc(N)nc(-c2c(Cl)cc3c(N4CC5CCC(C4)N5)nc(OC[C@@H]4CCCN4CCCCCCCN4CCC(c5cc(F)cc6c5CN(C5CCC(=O)NC5=O)C6=O)CC4)nc3c2F)c1C(F)(F)F ; Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cnc(-c4cc(O)cc5cccc(F)c45)c(F)c3n2)C(C)(C)C)cc1]

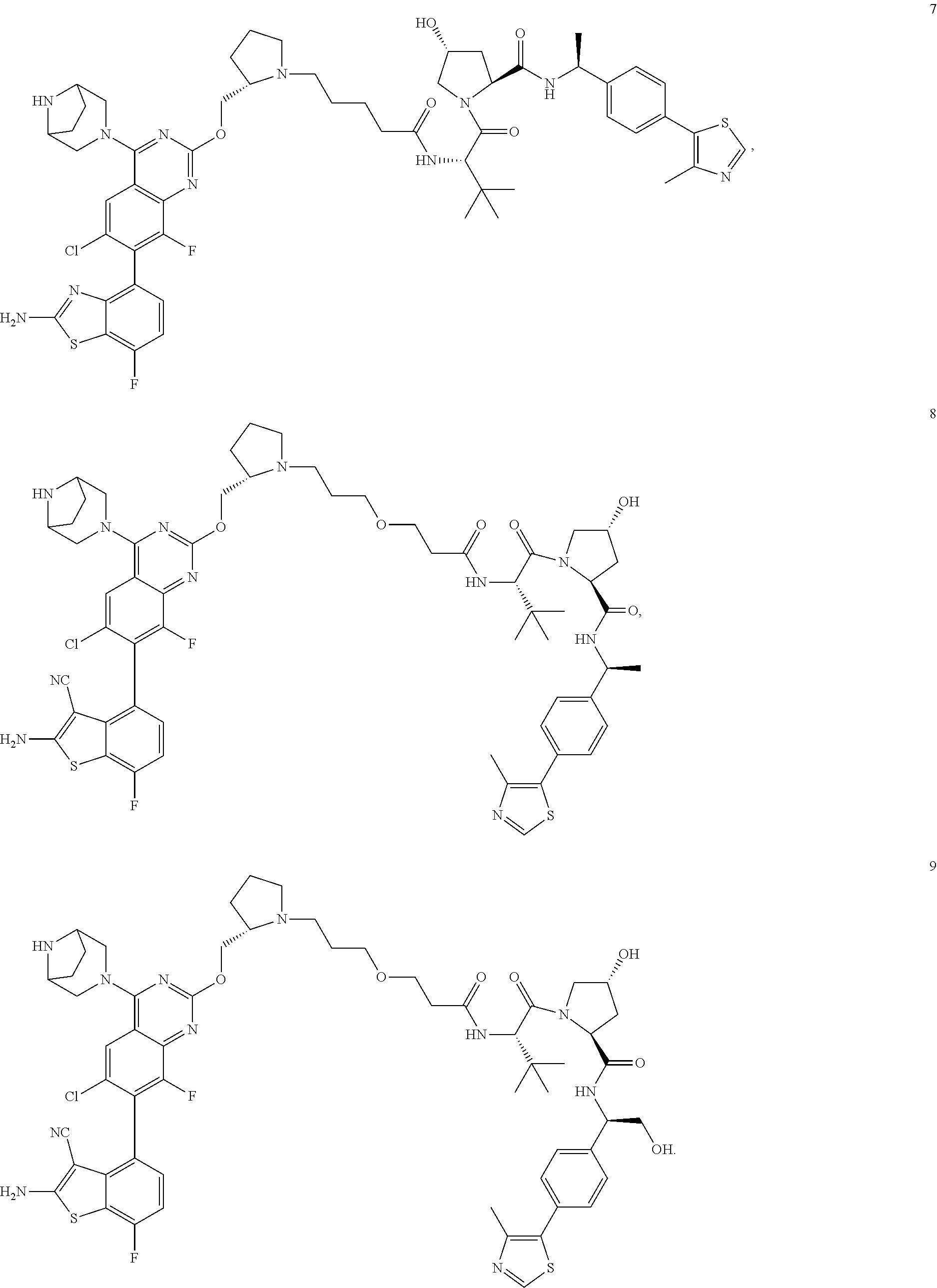 [SMILES: Cc1ncsc1-c1ccc([C@H](C)NC(=O)[C@@H]2C[C@@H](O)CN2C(=O)[C@@H](NC(=O)CCCCN2CCC[C@H]2COc2nc(N3CC4CCC(C3)N4)c3cc(Cl)c(-c4ccc(F)c5sc(N)nc45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cc(Cl)c(-c4ccc(F)c5sc(N)c(C#N)c45)c(F)c3n2)C(C)(C)C)cc1 ; Cc1ncsc1-c1ccc([C@H](CO)NC(=O)[C@@H]2C[C@@H](O)CN2C(=O)[C@@H](NC(=O)CCOCCCN2CCC[C@H]2COc2nc(N3CC4CCC(C3)N4)c3cc(Cl)c(-c4ccc(F)c5sc(N)c(C#N)c45)c(F)c3n2)C(C)(C)C)cc1]

Further conjugates include the following structures:

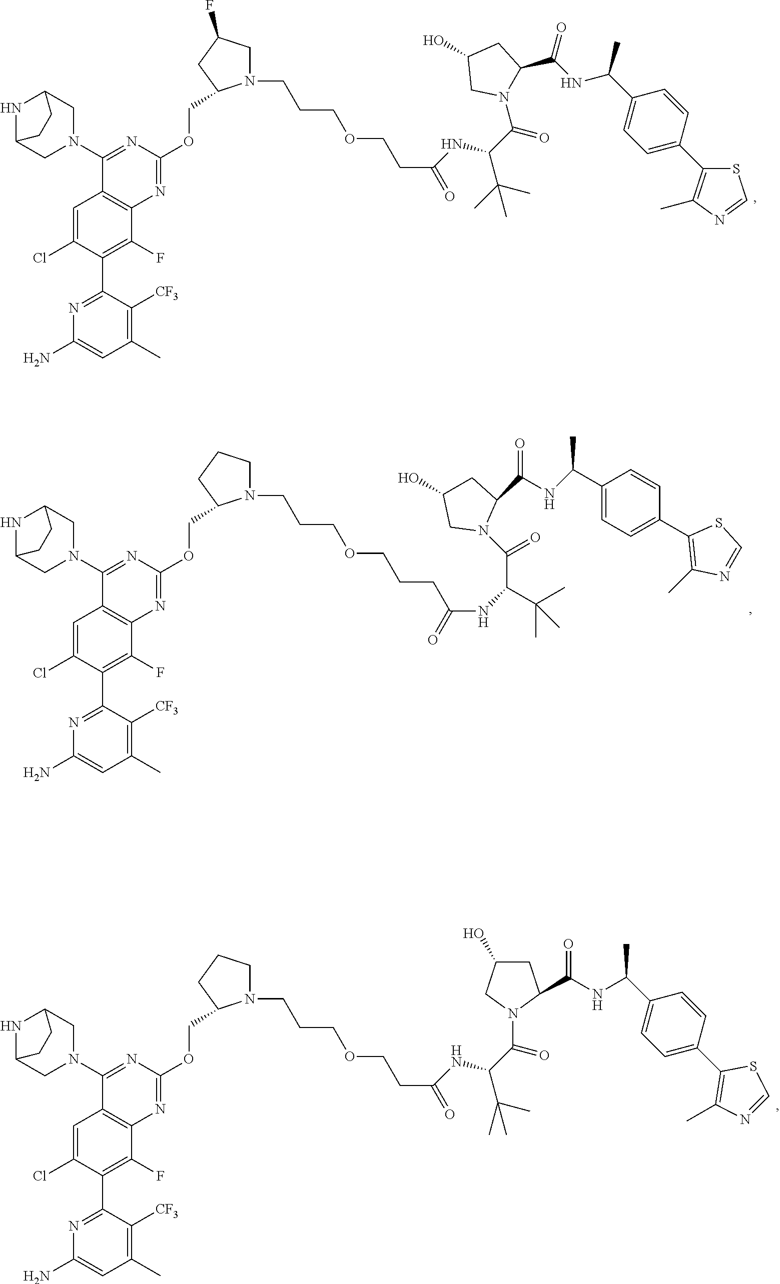 [SMILES: Cc1cc(N)nc(-c2c(Cl)cc3c(N4CC5CCC(C4)N5)nc(OC[C@@H]4C[C@@H](F)CN4CCCOCCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F ; Cc1cc(N)nc(-c2c(Cl)cc3c(N4CC5CCC(C4)N5)nc(OC[C@@H]4CCCN4CCCOCCCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F ; Cc1cc(N)nc(-c2c(Cl)cc3c(N4CC5CCC(C4)N5)nc(OC[C@@H]4CCCN4CCCOCCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F]

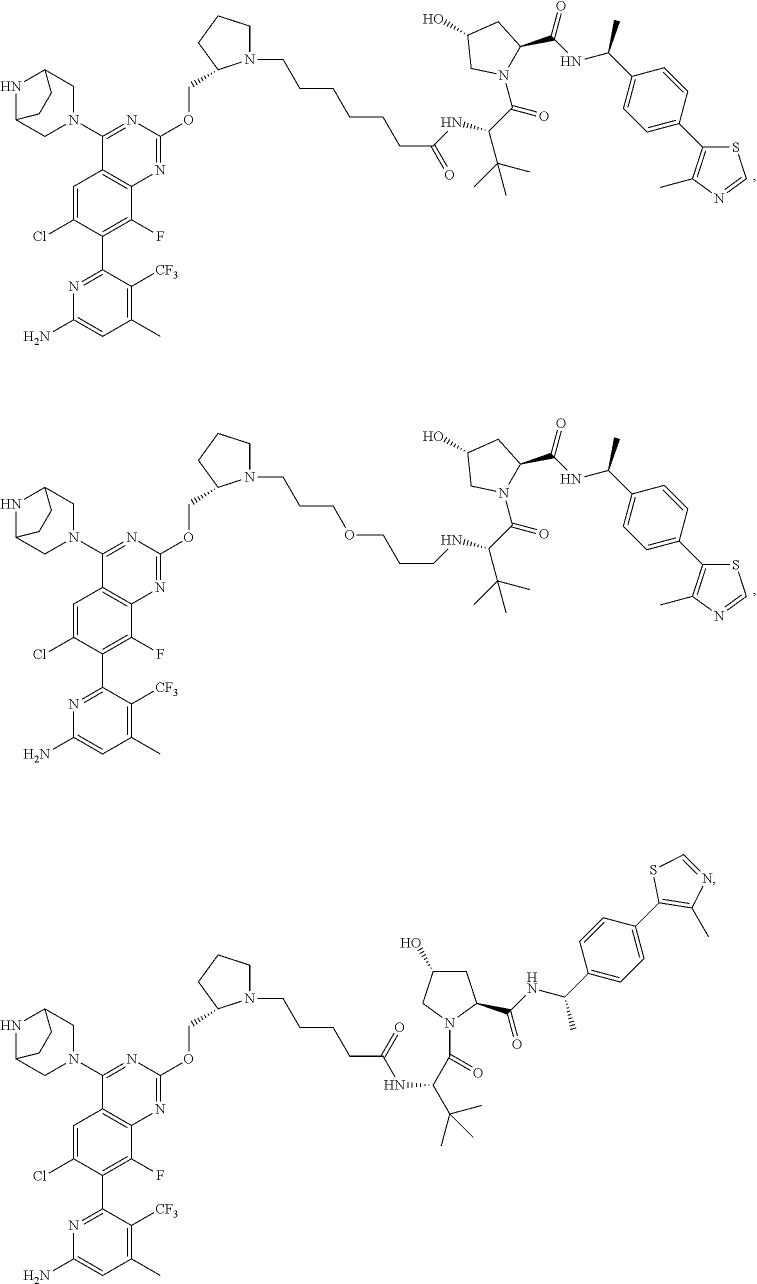 [SMILES: Cc1cc(N)nc(-c2c(Cl)cc3c(N4CC5CCC(C4)N5)nc(OC[C@@H]4CCCN4CCCCCCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F ; Cc1cc(N)nc(-c2c(Cl)cc3c(N4CC5CCC(C4)N5)nc(OC[C@@H]4CCCN4CCCOCCCN[C@H](C(=O)N4C[C@H](O)C[C@H]4C(=O)N[C@@H](C)c4ccc(-c5scnc5C)cc4)C(C)(C)C)nc3c2F)c1C(F)(F)F ; Cc1cc(N)nc(-c2c(Cl)cc3c(N4CC5CCC(C4)N5)nc(OC[C@@H]4CCCN4CCCCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F]

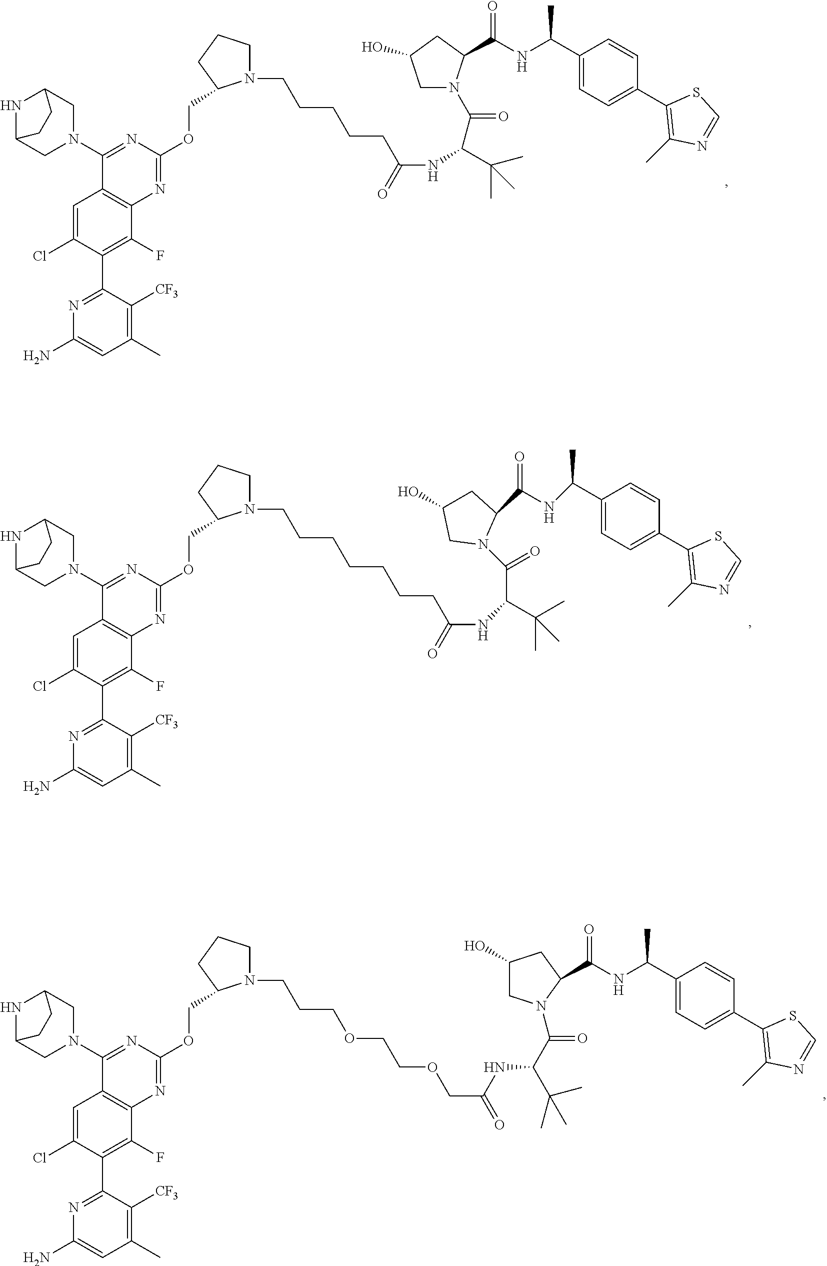 [SMILES: Cc1cc(N)nc(-c2c(Cl)cc3c(N4CC5CCC(C4)N5)nc(OC[C@@H]4CCCN4CCCCCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F ; Cc1cc(N)nc(-c2c(Cl)cc3c(N4CC5CCC(C4)N5)nc(OC[C@@H]4CCCN4CCCCCCCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F ; Cc1cc(N)nc(-c2c(Cl)cc3c(N4CC5CCC(C4)N5)nc(OC[C@@H]4CCCN4CCCOCCOCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F]

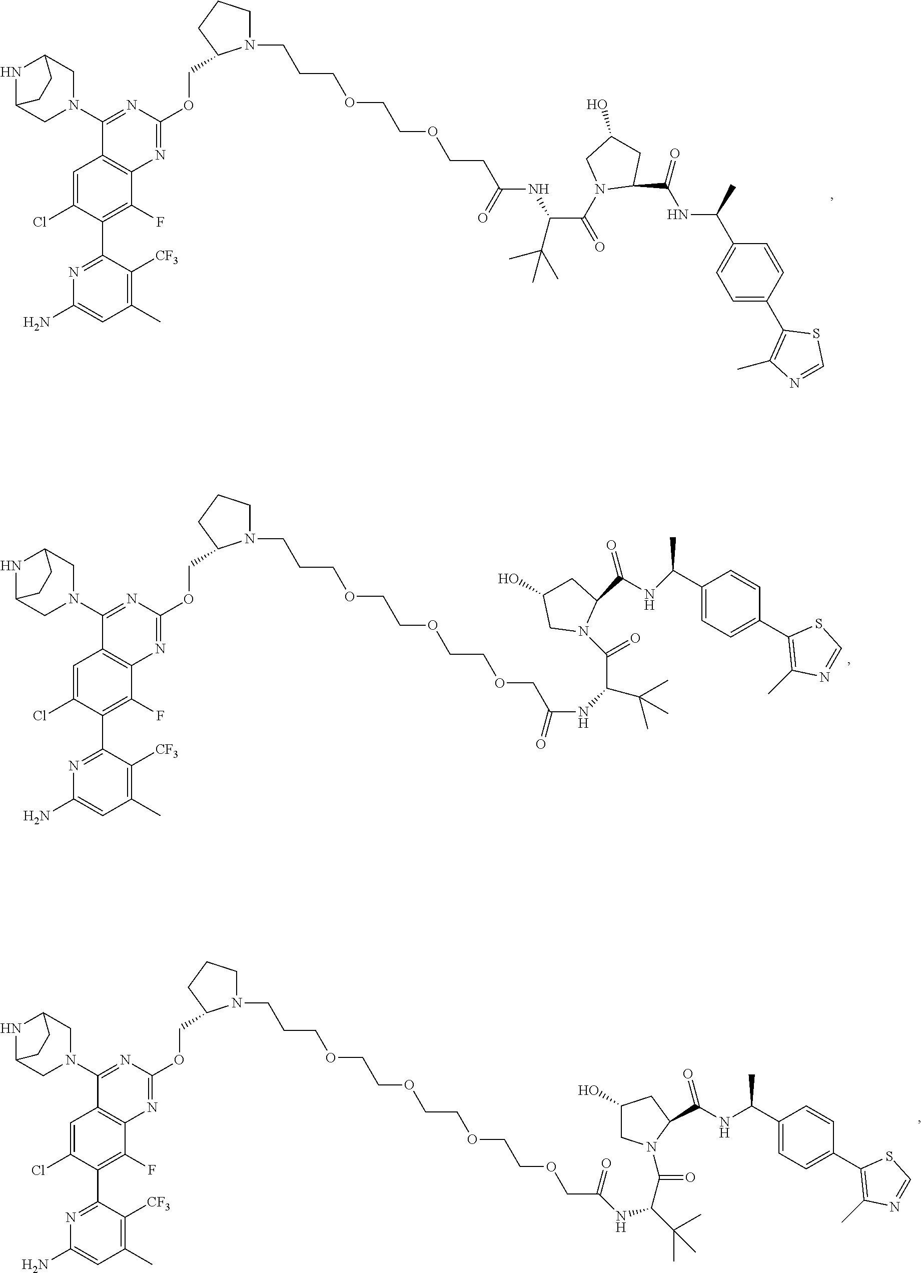 [SMILES: Cc1cc(N)nc(-c2c(Cl)cc3c(N4CC5CCC(C4)N5)nc(OC[C@@H]4CCCN4CCCOCCOCCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F ; Cc1cc(N)nc(-c2c(Cl)cc3c(N4CC5CCC(C4)N5)nc(OC[C@@H]4CCCN4CCCOCCOCCOCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F ; Cc1cc(N)nc(-c2c(Cl)cc3c(N4CC5CCC(C4)N5)nc(OC[C@@H]4CCCN4CCCOCCOCCOCCOCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F]

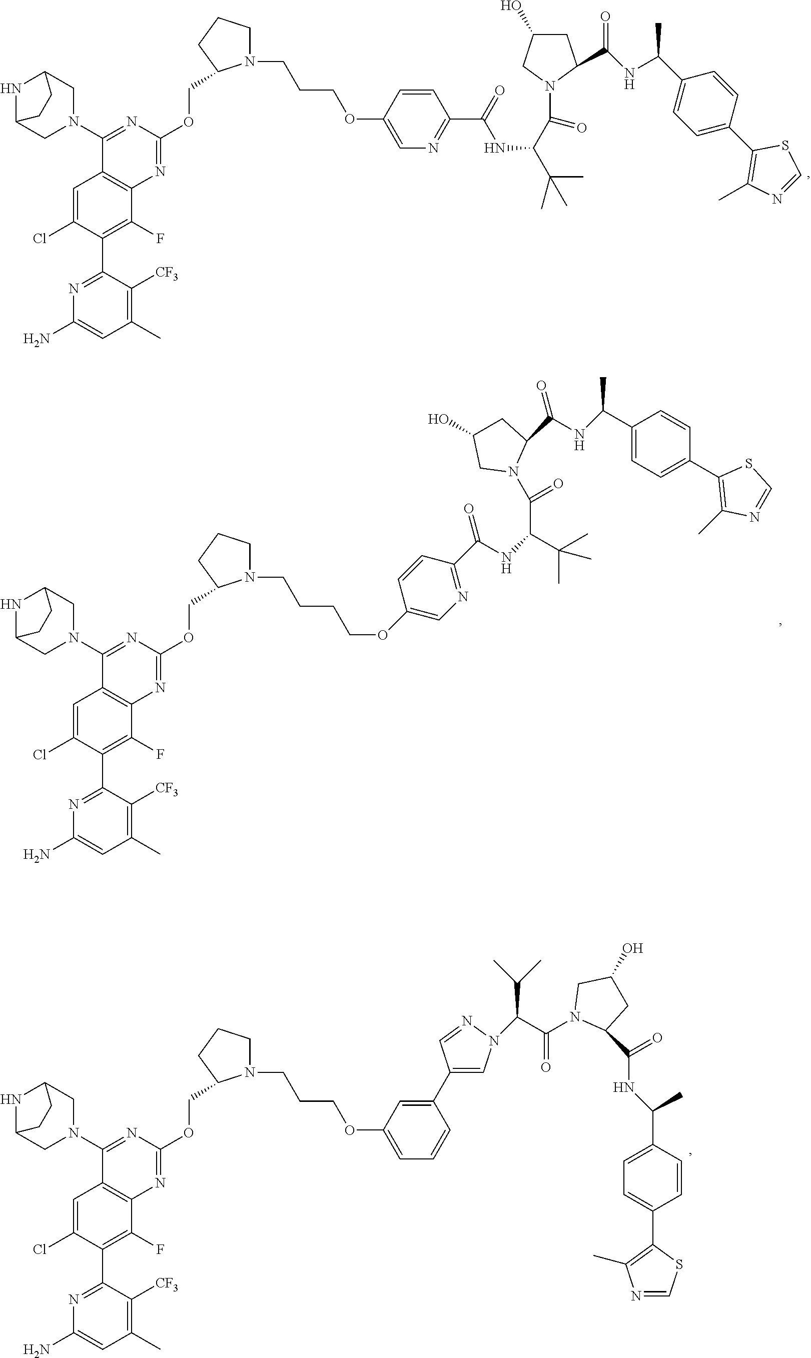

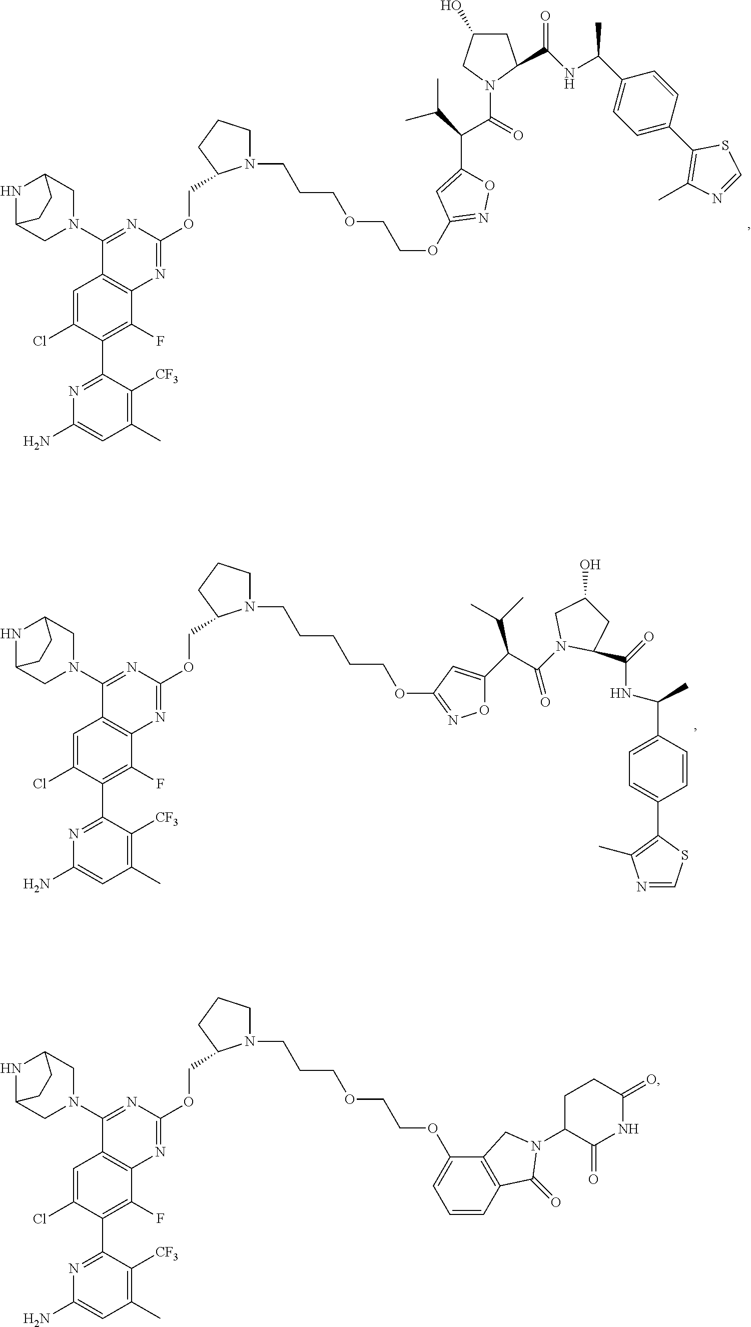 [SMILES: Cc1cc(N)nc(-c2c(Cl)cc3c(N4CC5CCC(C4)N5)nc(OC[C@@H]4CCCN4CCCOCCOc4cc([C@H](C(=O)N5C[C@H](O)C[C@H]5C(=O)N[C@@H](C)c5ccc(-c6scnc6C)cc5)C(C)C)on4)nc3c2F)c1C(F)(F)F ; Cc1cc(N)nc(-c2c(Cl)cc3c(N4CC5CCC(C4)N5)nc(OC[C@@H]4CCCN4CCCCCOc4cc([C@H](C(=O)N5C[C@H](O)C[C@H]5C(=O)N[C@@H](C)c5ccc(-c6scnc6C)cc5)C(C)C)on4)nc3c2F)c1C(F)(F)F ; Cc1cc(N)nc(-c2c(Cl)cc3c(N4CC5CCC(C4)N5)nc(OC[C@@H]4CCCN4CCCOCCOc4cccc5c4CN(C4CCC(=O)NC4=O)C5=O)nc3c2F)c1C(F)(F)F]

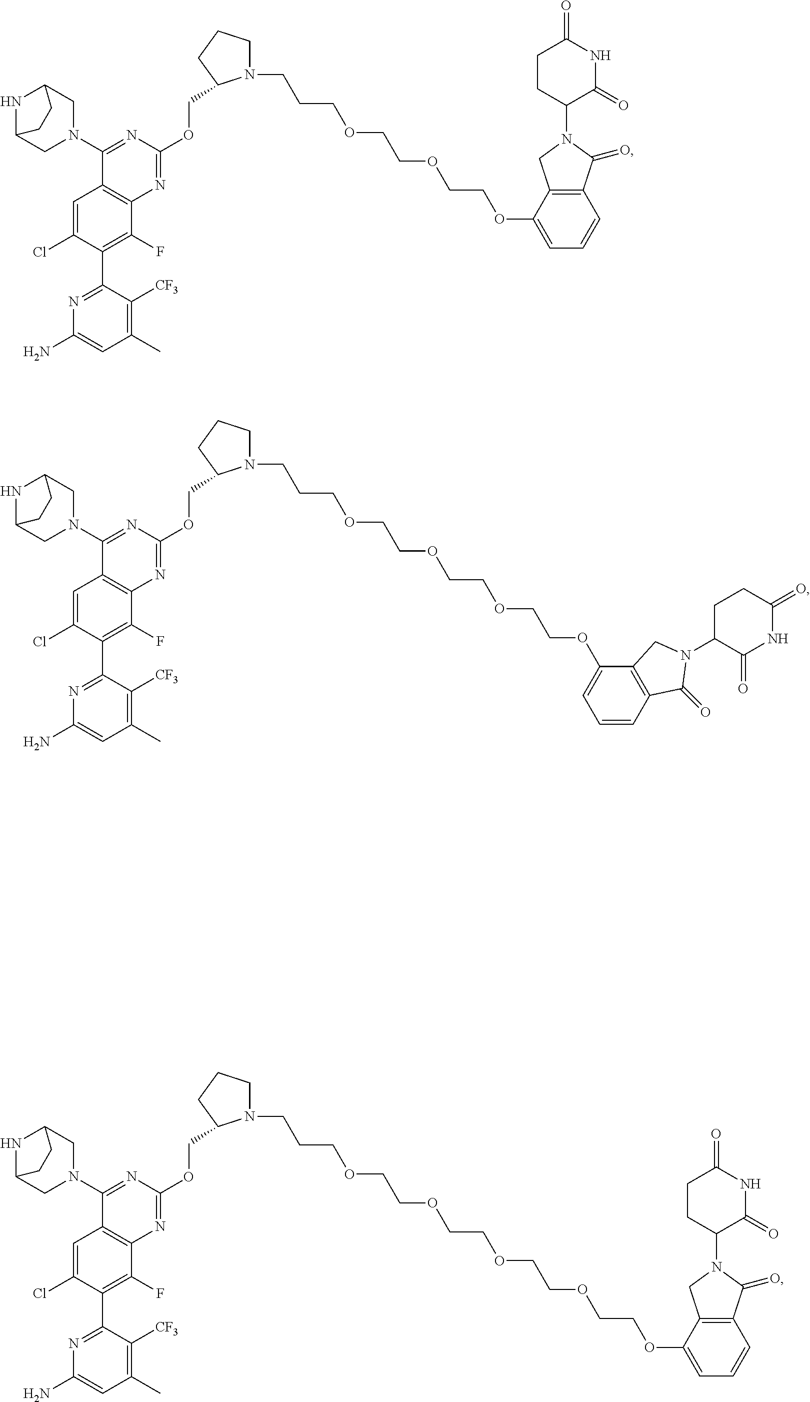 [SMILES: Cc1cc(N)nc(-c2c(Cl)cc3c(N4CC5CCC(C4)N5)nc(OC[C@@H]4CCCN4CCCOCCOCCOc4cccc5c4CN(C4CCC(=O)NC4=O)C5=O)nc3c2F)c1C(F)(F)F ; Cc1cc(N)nc(-c2c(Cl)cc3c(N4CC5CCC(C4)N5)nc(OC[C@@H]4CCCN4CCCOCCOCCOCCOc4cccc5c4CN(C4CCC(=O)NC4=O)C5=O)nc3c2F)c1C(F)(F)F ; Cc1cc(N)nc(-c2c(Cl)cc3c(N4CC5CCC(C4)N5)nc(OC[C@@H]4CCCN4CCCOCCOCCOCCOCCOc4cccc5c4CN(C4CCC(=O)NC4=O)C5=O)nc3c2F)c1C(F)(F)F]

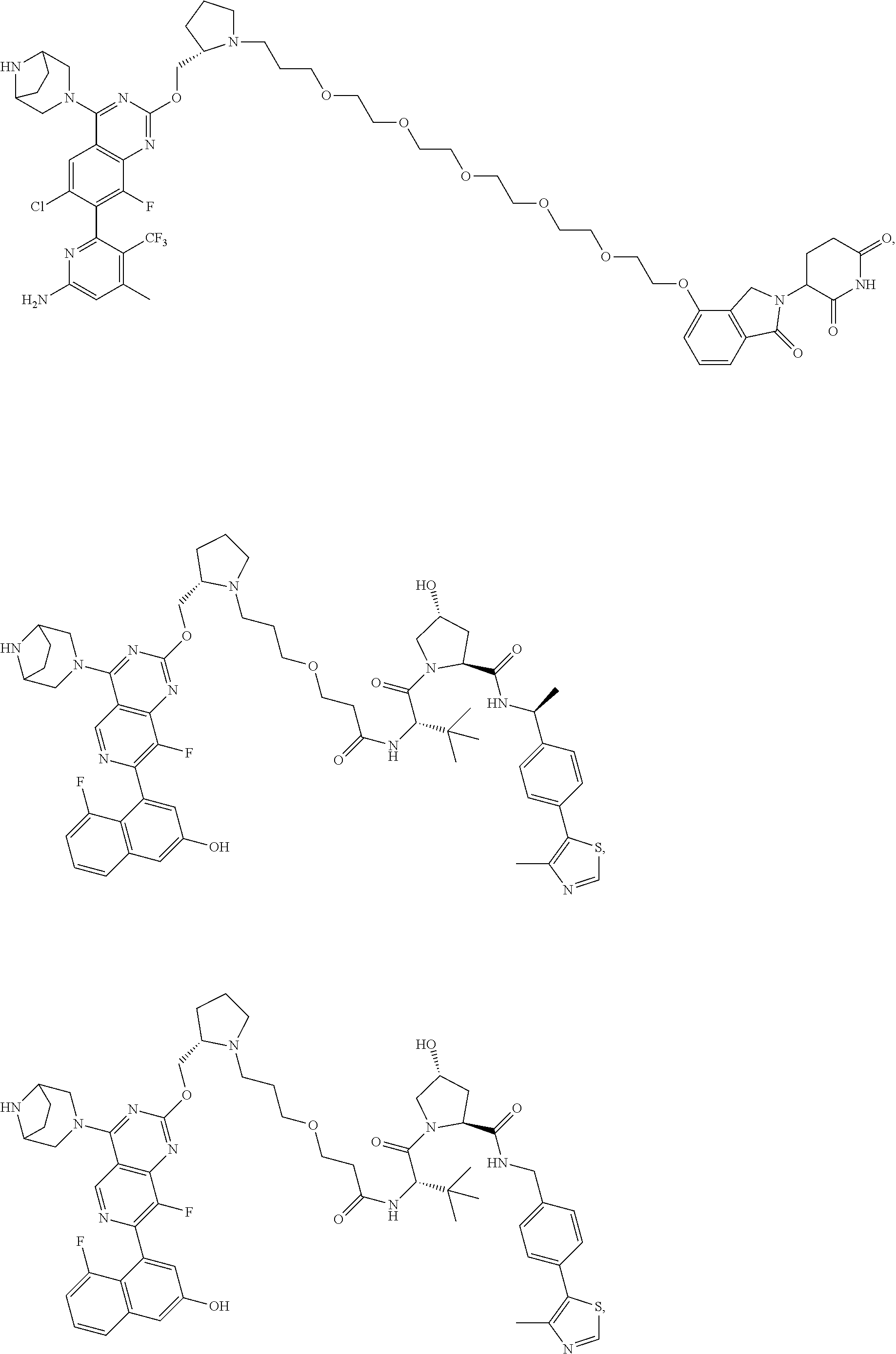 [SMILES: Cc1cc(N)nc(-c2c(Cl)cc3c(N4CC5CCC(C4)N5)nc(OC[C@@H]4CCCN4CCCOCCOCCOCCOCCOCCOc4cccc5c4CN(C4CCC(=O)NC4=O)C5=O)nc3c2F)c1C(F)(F)F ; Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cnc(-c4cc(O)cc5cccc(F)c45)c(F)c3n2)C(C)(C)C)cc1 ; Cc1ncsc1-c1ccc(CNC(=O)[C@@H]2C[C@@H](O)CN2C(=O)[C@@H](NC(=O)CCOCCCN2CCC[C@H]2COc2nc(N3CC4CCC(C3)N4)c3cnc(-c4cc(O)cc5cccc(F)c45)c(F)c3n2)C(C)(C)C)cc1]

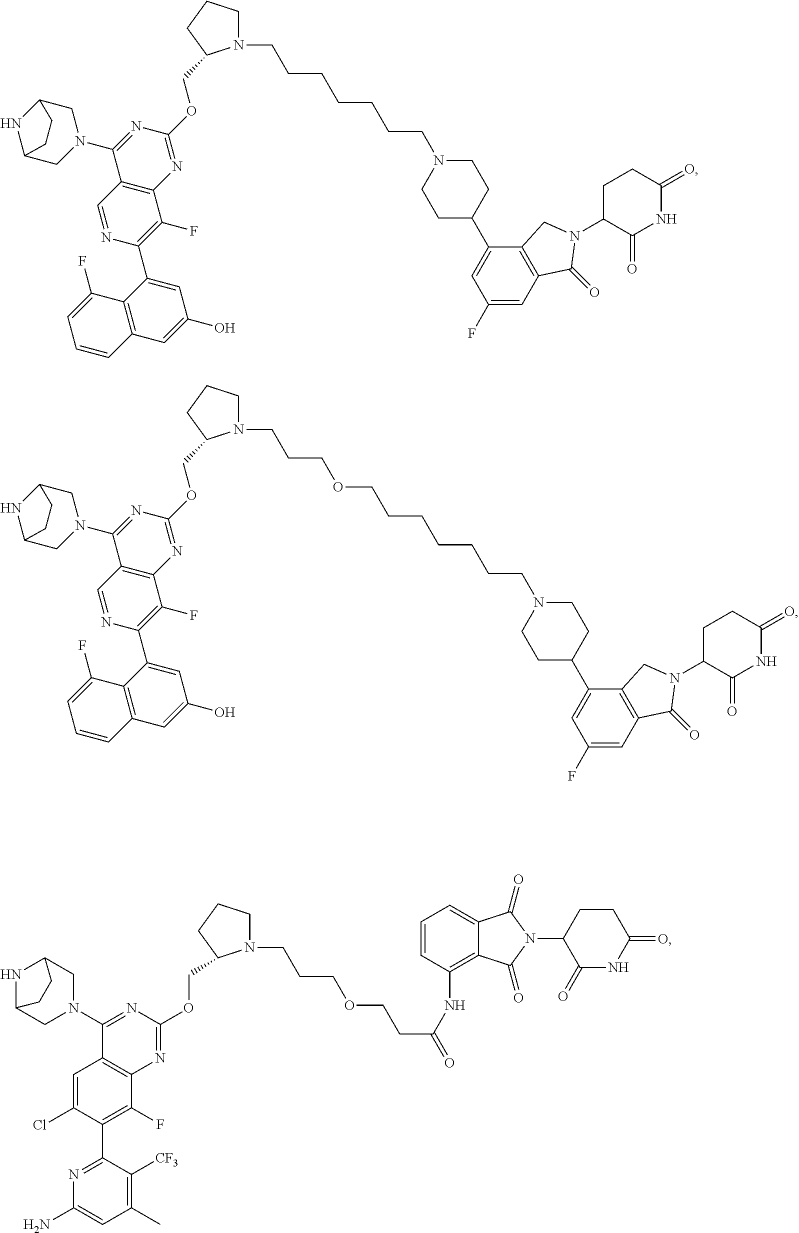 [SMILES: O=C1CCC(N2Cc3c(cc(F)cc3C3CCN(CCCCCCCN4CCC[C@H]4COc4nc(N5CC6CCC(C5)N6)c5cnc(-c6cc(O)cc7cccc(F)c67)c(F)c5n4)CC3)C2=O)C(=O)N1 ; O=C1CCC(N2Cc3c(cc(F)cc3C3CCN(CCCCCCCOCCCN4CCC[C@H]4COc4nc(N5CC6CCC(C5)N6)c5cnc(-c6cc(O)cc7cccc(F)c67)c(F)c5n4)CC3)C2=O)C(=O)N1 ; Cc1cc(N)nc(-c2c(Cl)cc3c(N4CC5CCC(C4)N5)nc(OC[C@@H]4CCCN4CCCOCCC(=O)Nc4cccc5c4C(=O)N(C4CCC(=O)NC4=O)C5=O)nc3c2F)c1C(F)(F)F]

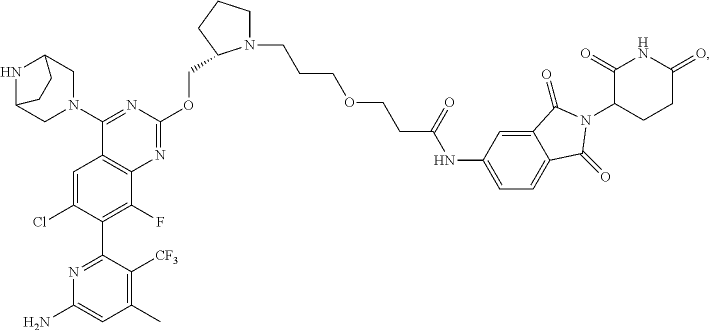 [SMILES: Cc1cc(N)nc(-c2c(Cl)cc3c(N4CC5CCC(C4)N5)nc(OC[C@@H]4CCCN4CCCOCCC(=O)Nc4ccc5c(c4)C(=O)N(C4CCC(=O)NC4=O)C5=O)nc3c2F)c1C(F)(F)F]

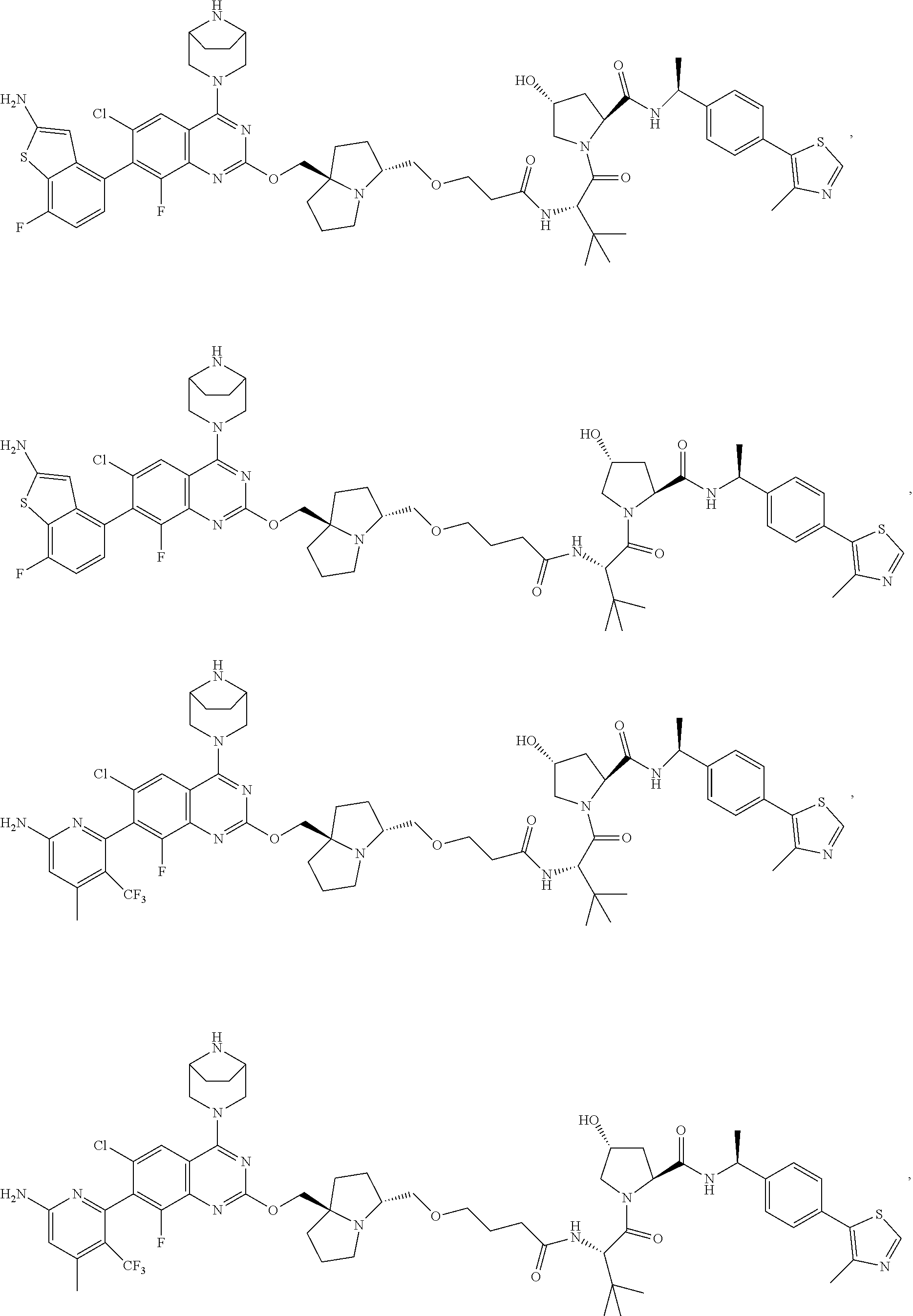

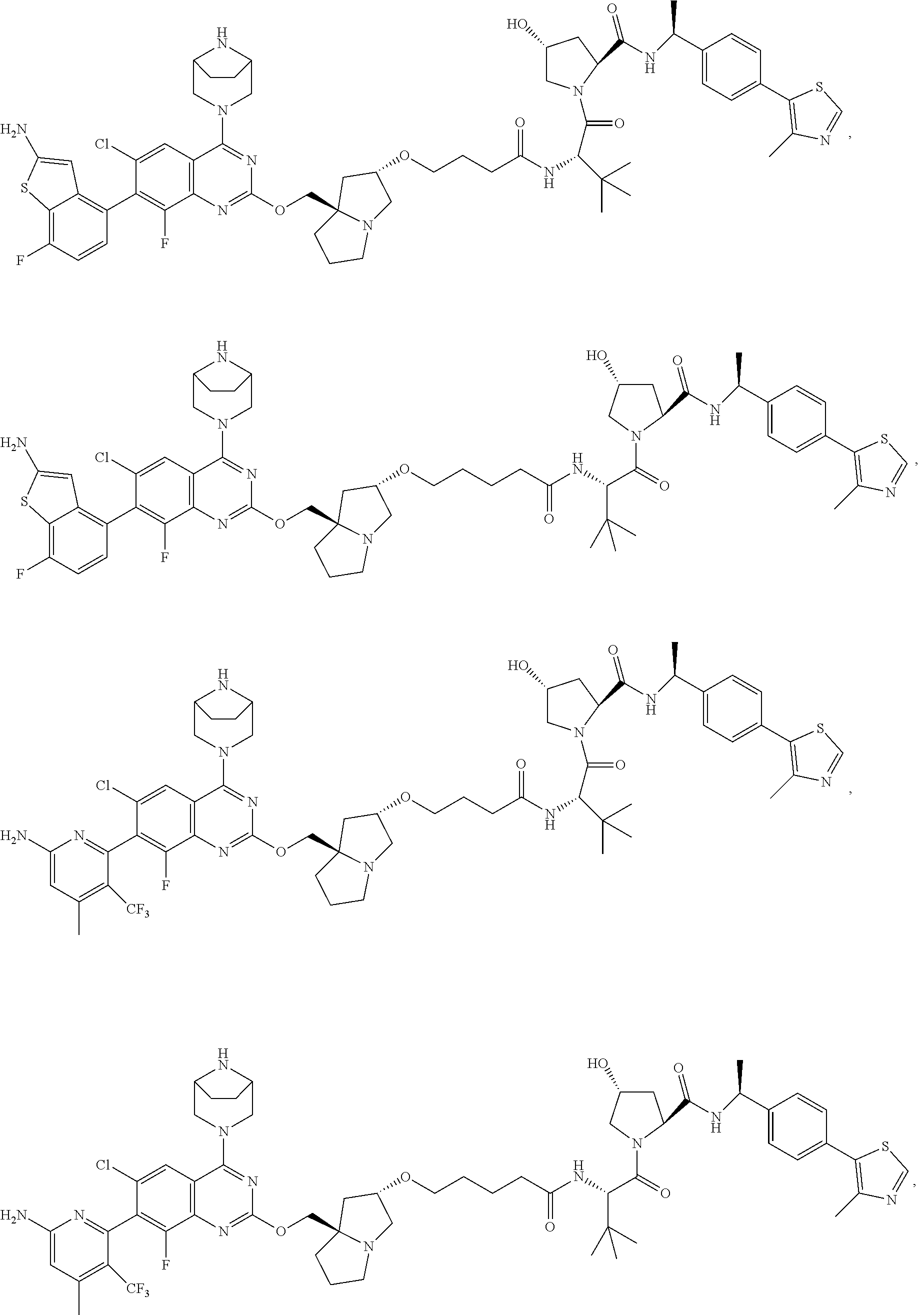

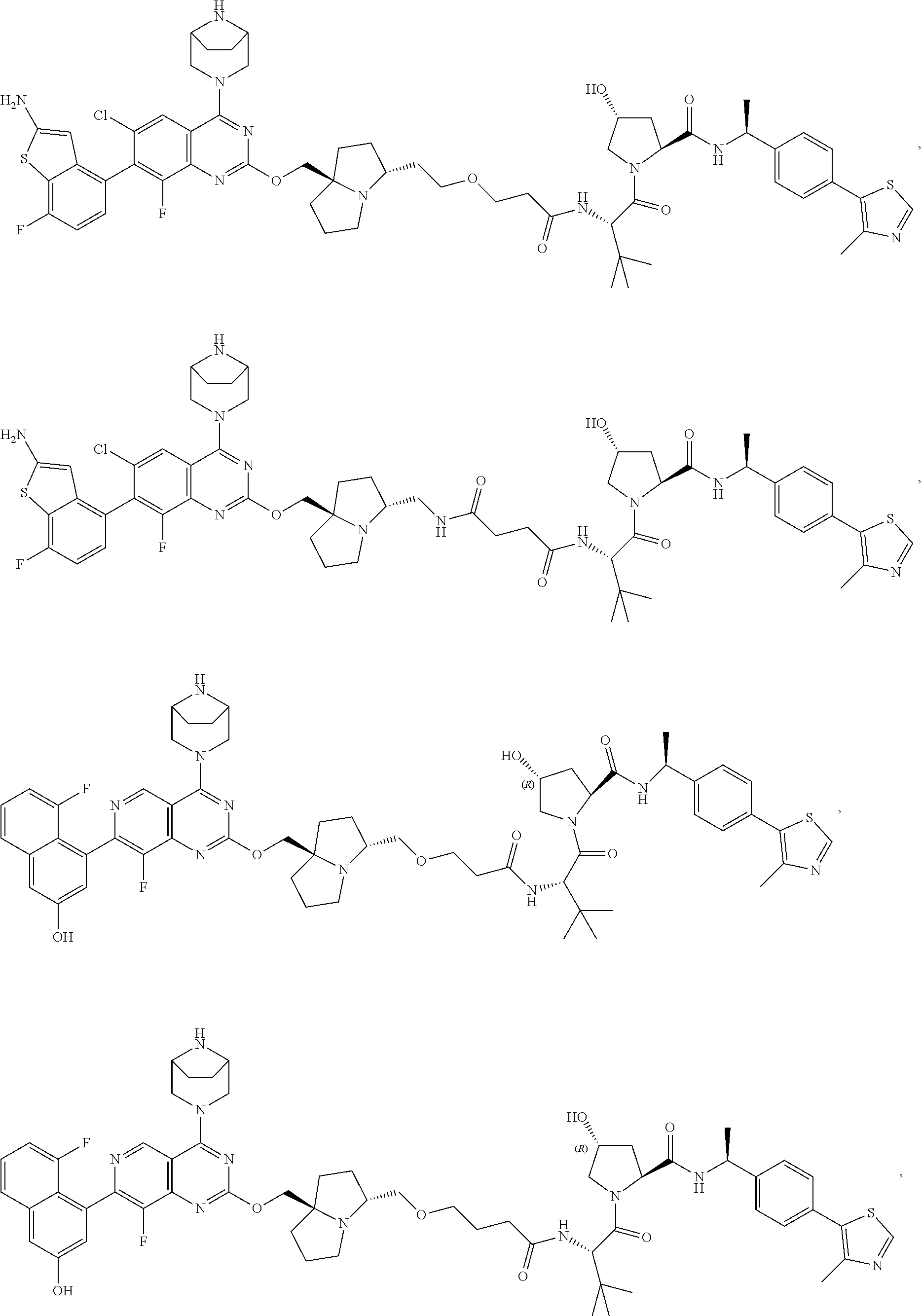

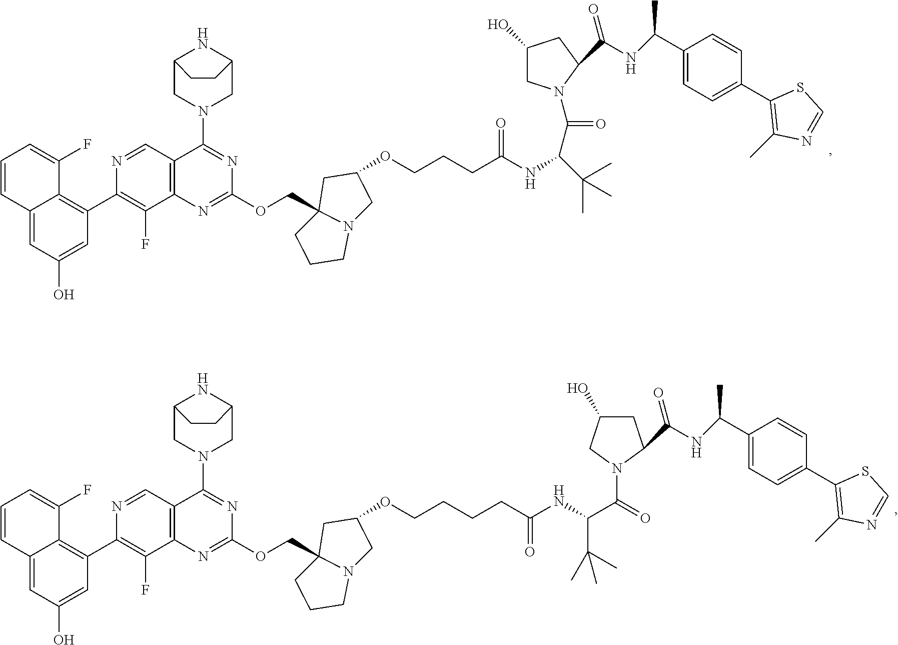 [SMILES: Cc1ncsc1-c1ccc([C@H](C)NC(=O)[C@@H]2C[C@@H](O)CN2C(=O)[C@@H](NC(=O)CCCO[C@@H]2CN3CCC[C@]3(COc3nc(N4CC5CCC(C4)N5)c4cnc(-c5cc(O)cc6cccc(F)c56)c(F)c4n3)C2)C(C)(C)C)cc1 ; Cc1ncsc1-c1ccc([C@H](C)NC(=O)[C@@H]2C[C@@H](O)CN2C(=O)[C@@H](NC(=O)CCCCO[C@@H]2CN3CCC[C@]3(COc3nc(N4CC5CCC(C4)N5)c4cnc(-c5cc(O)cc6cccc(F)c56)c(F)c4n3)C2)C(C)(C)C)cc1]

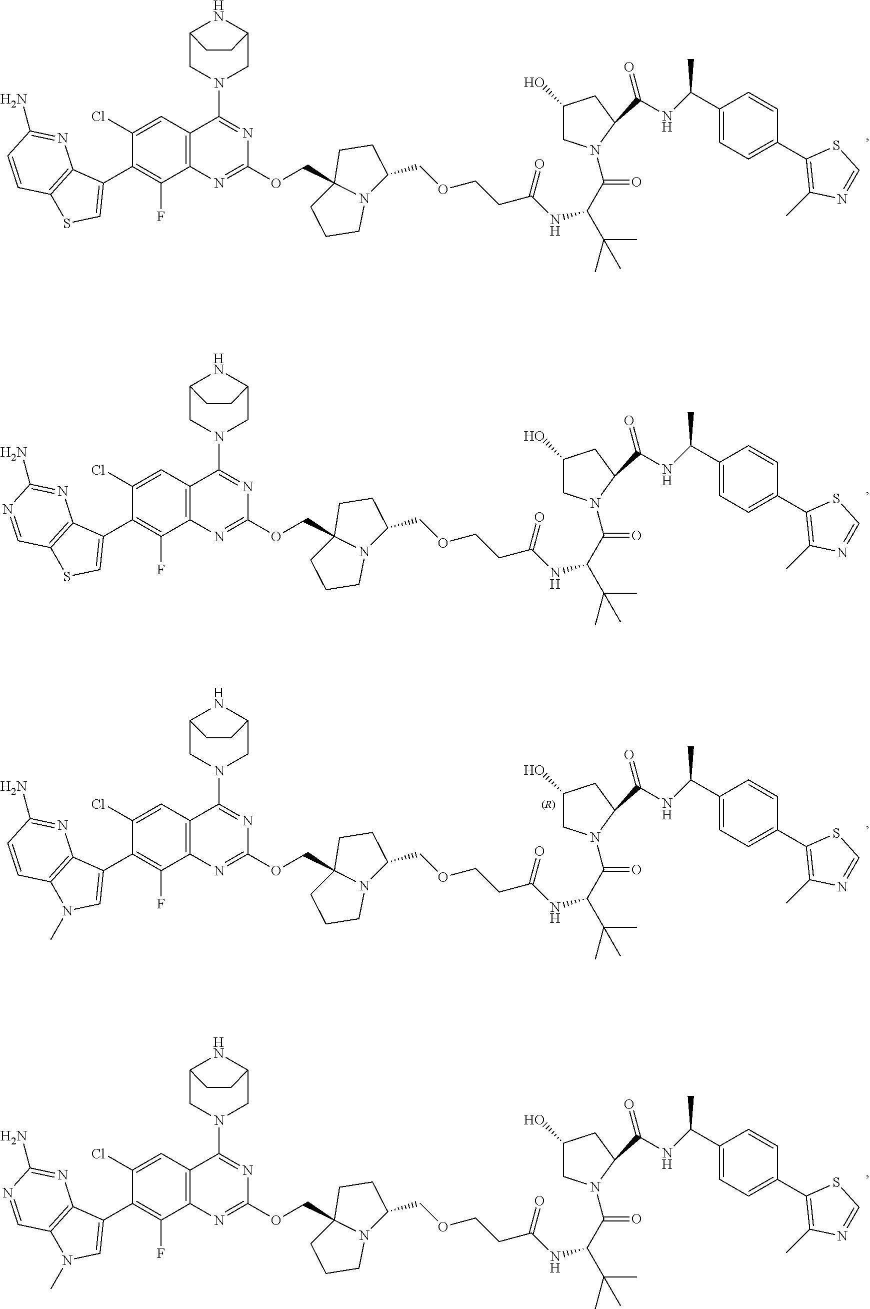

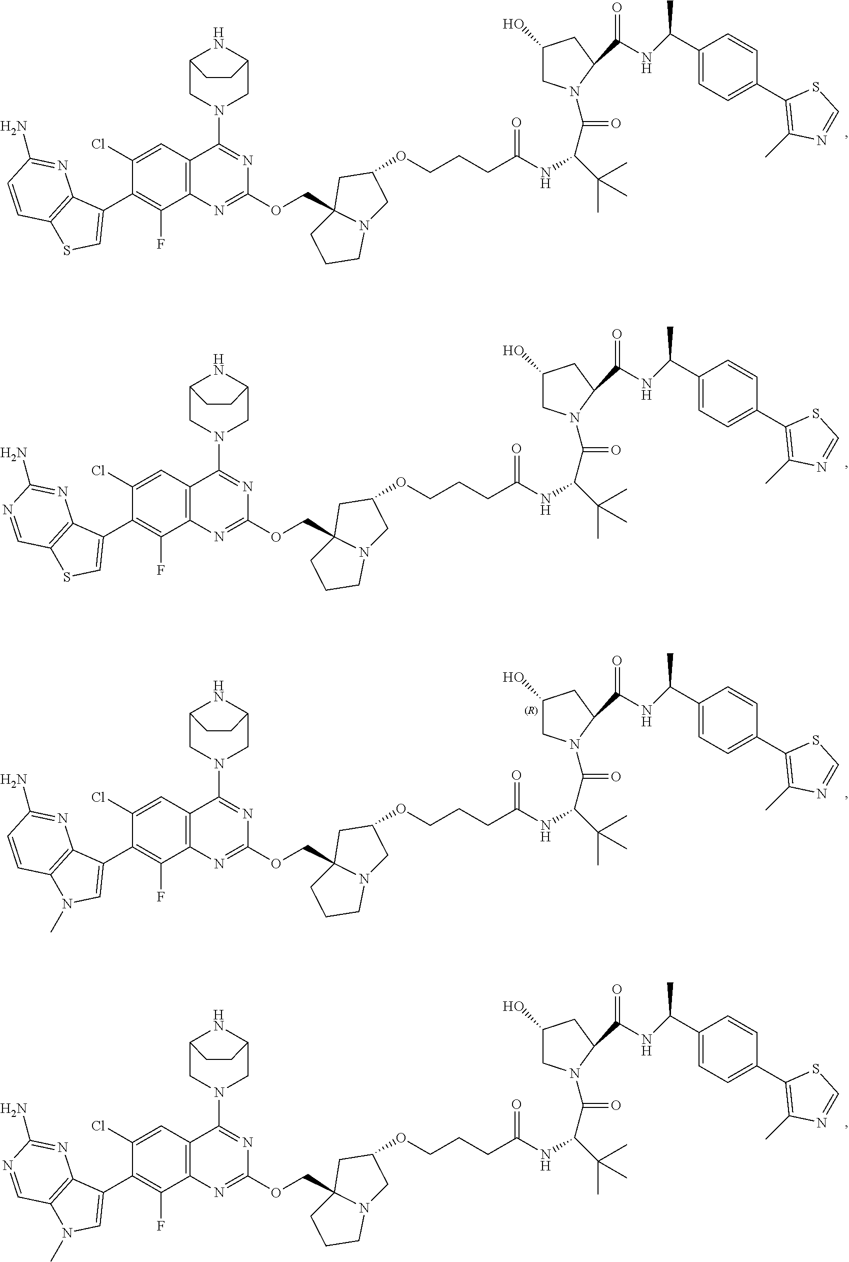

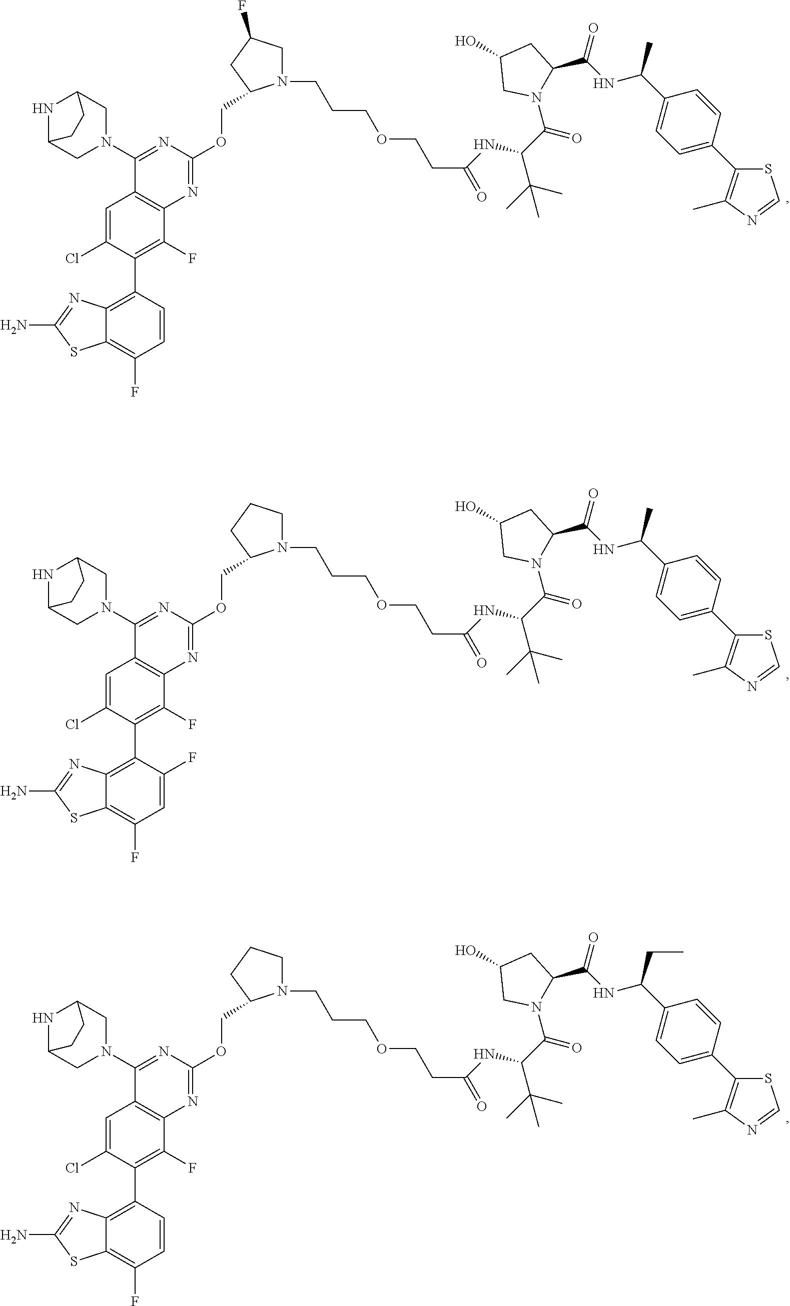 [SMILES: Cc1ncsc1-c1ccc([C@H](C)NC(=O)[C@@H]2C[C@@H](O)CN2C(=O)[C@@H](NC(=O)CCOCCCN2C[C@H](F)C[C@H]2COc2nc(N3CC4CCC(C3)N4)c3cc(Cl)c(-c4ccc(F)c5sc(N)nc45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cc(Cl)c(-c4c(F)cc(F)c5sc(N)nc45)c(F)c3n2)C(C)(C)C)cc1 ; CC[C@H](NC(=O)[C@@H]1C[C@@H](O)CN1C(=O)[C@@H](NC(=O)CCOCCCN1CCC[C@H]1COc1nc(N2CC3CCC(C2)N3)c2cc(Cl)c(-c3ccc(F)c4sc(N)nc34)c(F)c2n1)C(C)(C)C)c1ccc(-c2scnc2C)cc1]

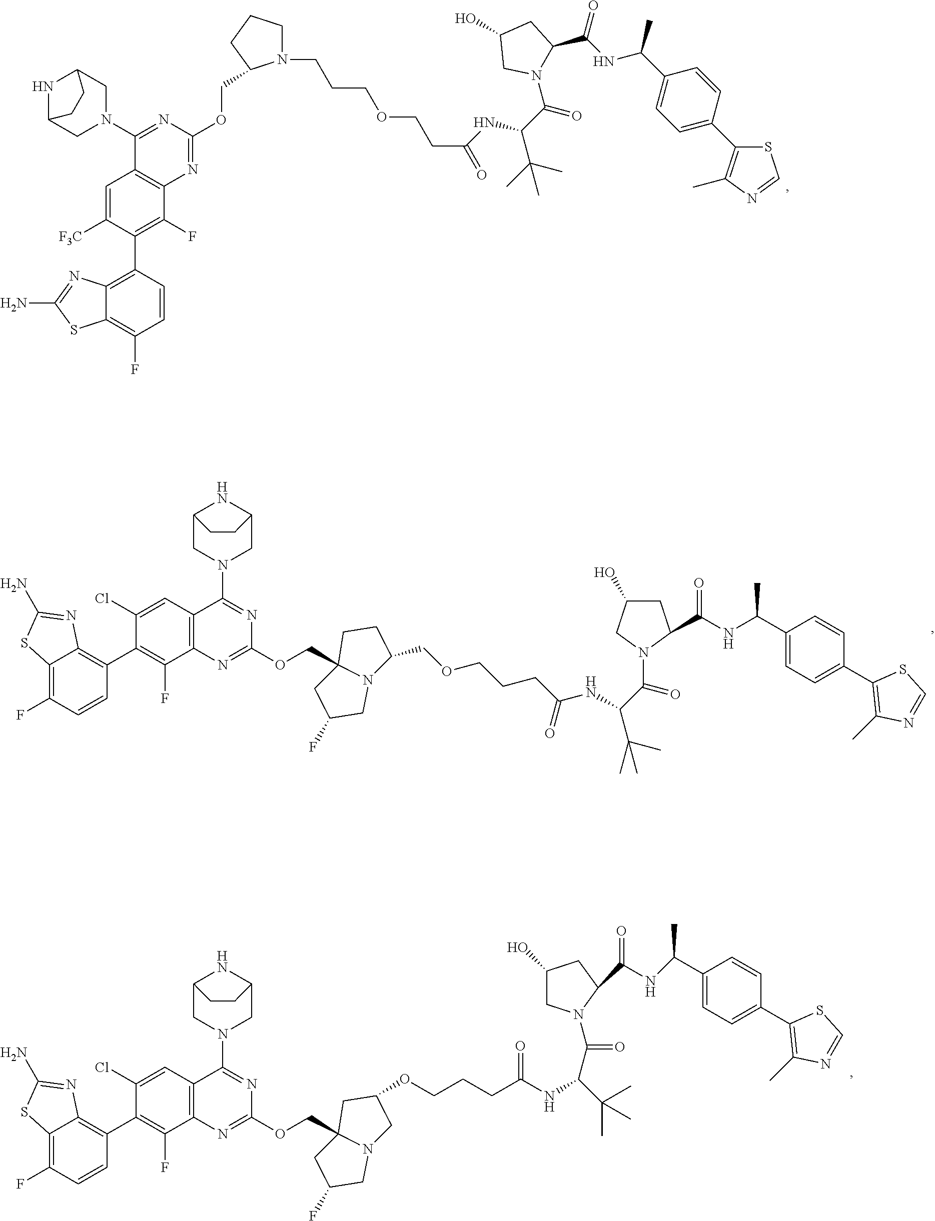

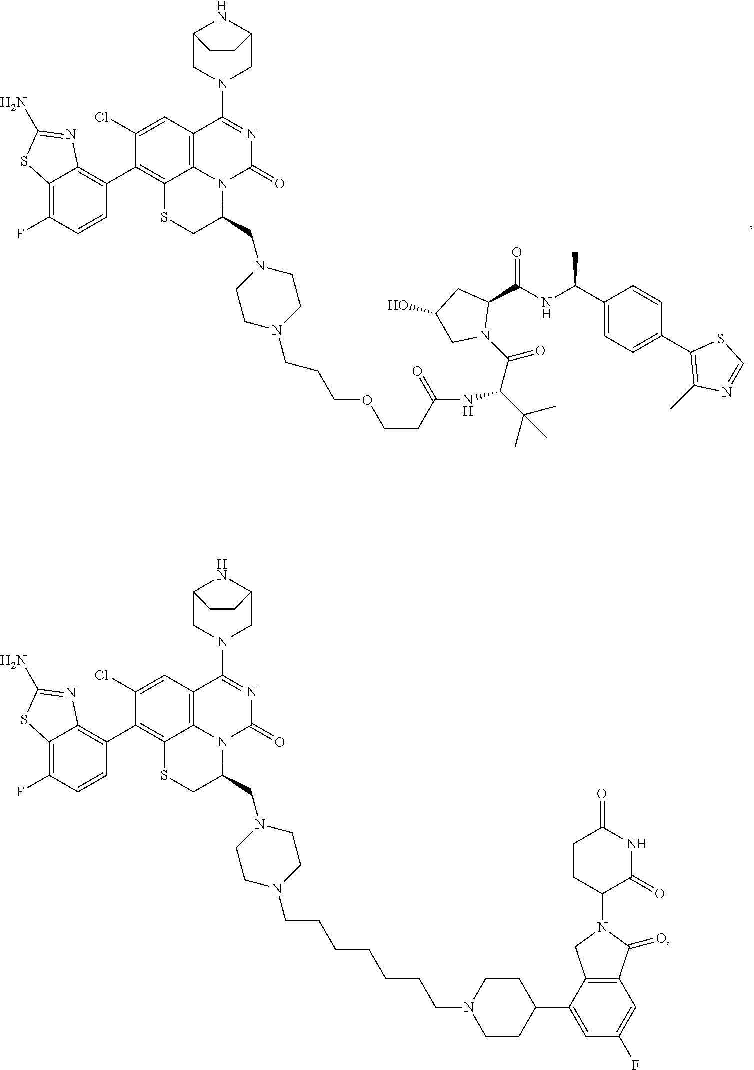 [SMILES: Cc1ncsc1-c1ccc([C@H](C)NC(=O)[C@@H]2C[C@@H](O)CN2C(=O)[C@@H](NC(=O)CCOCCCN2CCN(C[C@H]3CSc4c(-c5ccc(F)c6sc(N)nc56)c(Cl)cc5c(N6CC7CCC(C6)N7)nc(=O)n3c45)CC2)C(C)(C)C)cc1 ; Nc1nc2c(-c3c(Cl)cc4c(N5CC6CCC(C5)N6)nc(=O)n5c4c3SC[C@@H]5CN3CCN(CCCCCCCN4CCC(c5cc(F)cc6c5CN(C5CCC(=O)NC5=O)C6=O)CC4)CC3)ccc(F)c2s1]

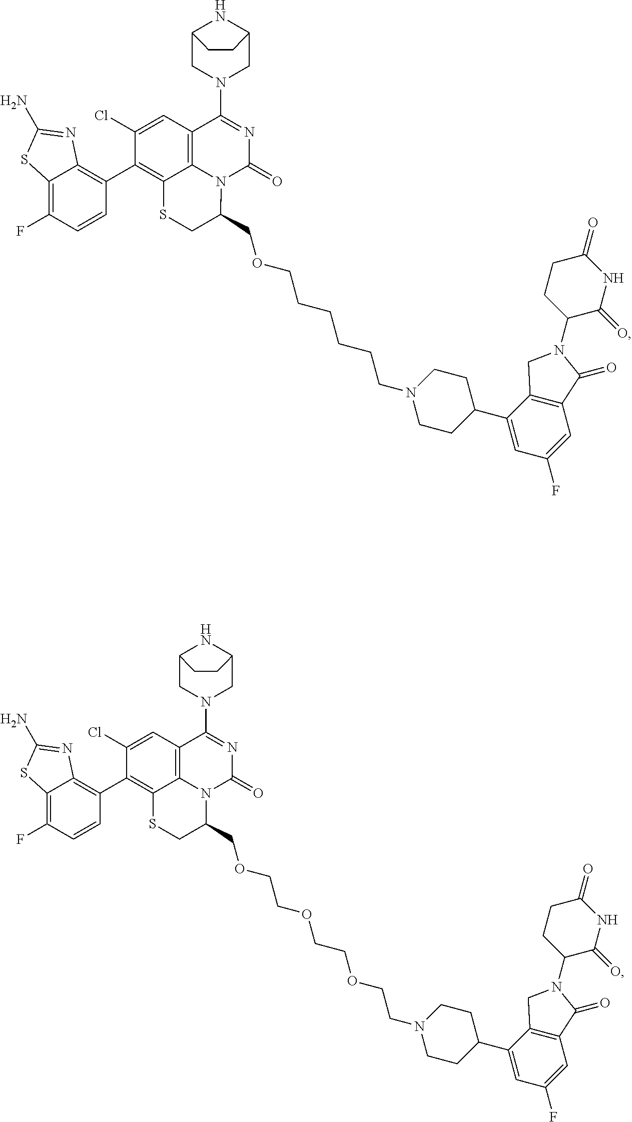 [SMILES: Nc1nc2c(-c3c(Cl)cc4c(N5CC6CCC(C5)N6)nc(=O)n5c4c3SC[C@@H]5COCCCCCCN3CCC(c4cc(F)cc5c4CN(C4CCC(=O)NC4=O)C5=O)CC3)ccc(F)c2s1 ; Nc1nc2c(-c3c(Cl)cc4c(N5CC6CCC(C5)N6)nc(=O)n5c4c3SC[C@@H]5COCCOCCOCCN3CCC(c4cc(F)cc5c4CN(C4CCC(=O)NC4=O)C5=O)CC3)ccc(F)c2s1]

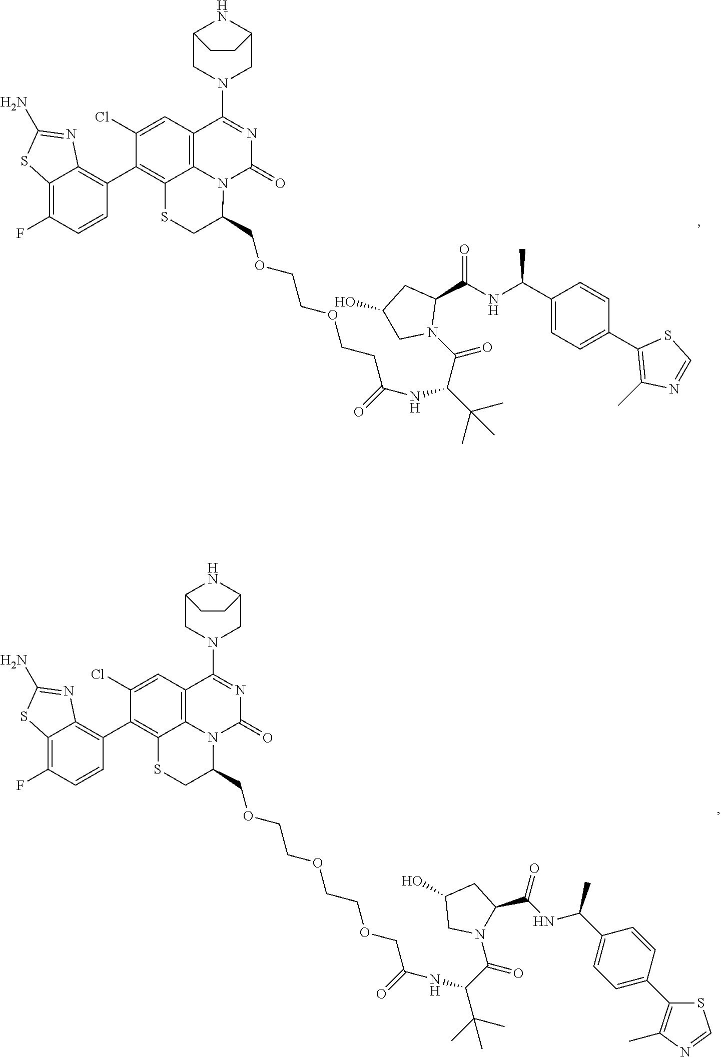 [SMILES: Cc1ncsc1-c1ccc([C@H](C)NC(=O)[C@@H]2C[C@@H](O)CN2C(=O)[C@@H](NC(=O)CCOCCOC[C@H]2CSc3c(-c4ccc(F)c5sc(N)nc45)c(Cl)cc4c(N5CC6CCC(C5)N6)nc(=O)n2c34)C(C)(C)C)cc1 ; Cc1ncsc1-c1ccc([C@H](C)NC(=O)[C@@H]2C[C@@H](O)CN2C(=O)[C@@H](NC(=O)COCCOCCOC[C@H]2CSc3c(-c4ccc(F)c5sc(N)nc45)c(Cl)cc4c(N5CC6CCC(C5)N6)nc(=O)n2c34)C(C)(C)C)cc1]

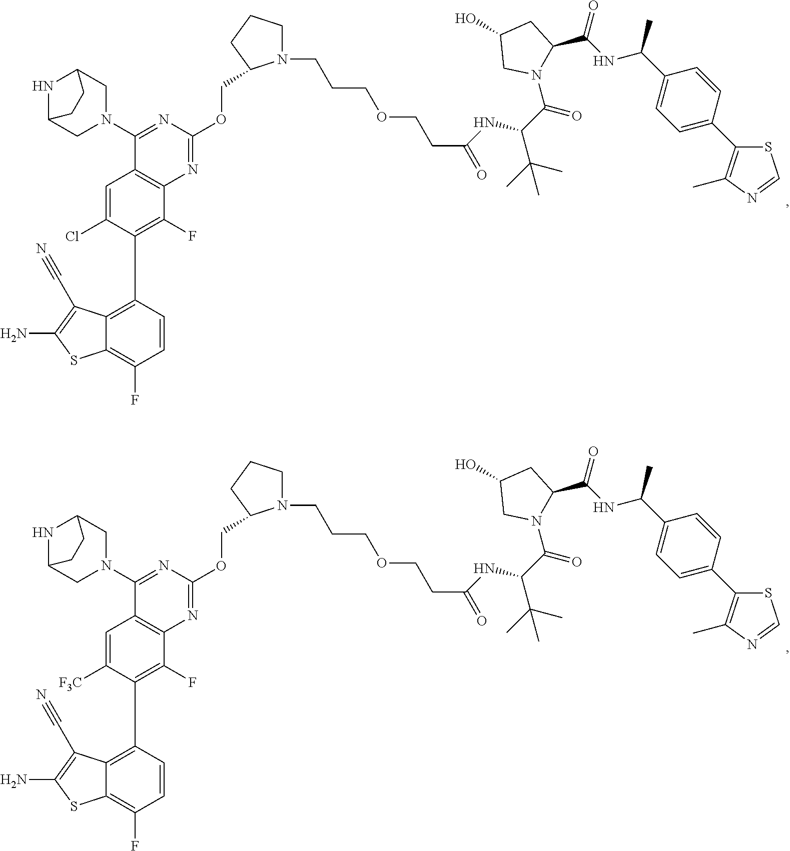 [SMILES: Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cc(Cl)c(-c4ccc(F)c5sc(N)c(C#N)c45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cc(C(F)(F)F)c(-c4ccc(F)c5sc(N)c(C#N)c45)c(F)c3n2)C(C)(C)C)cc1]

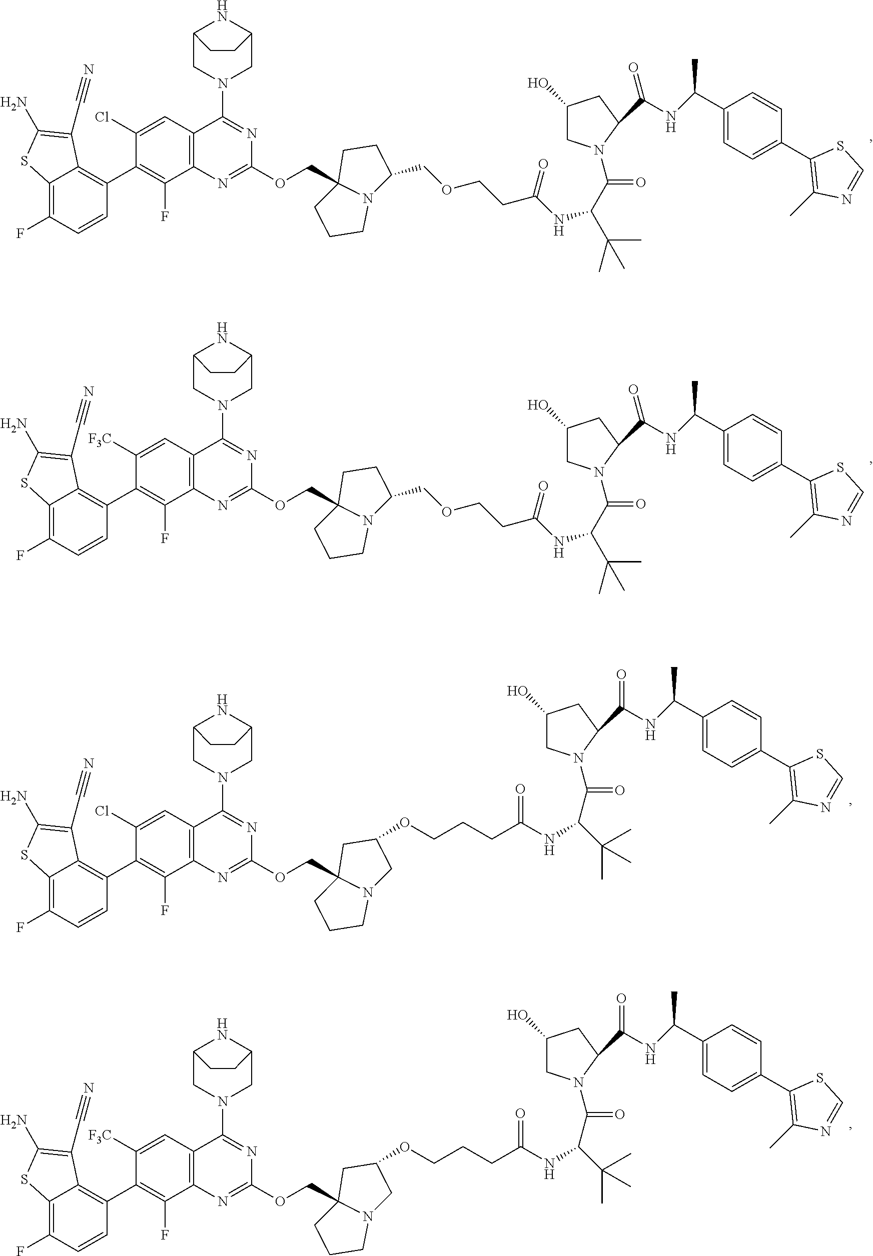

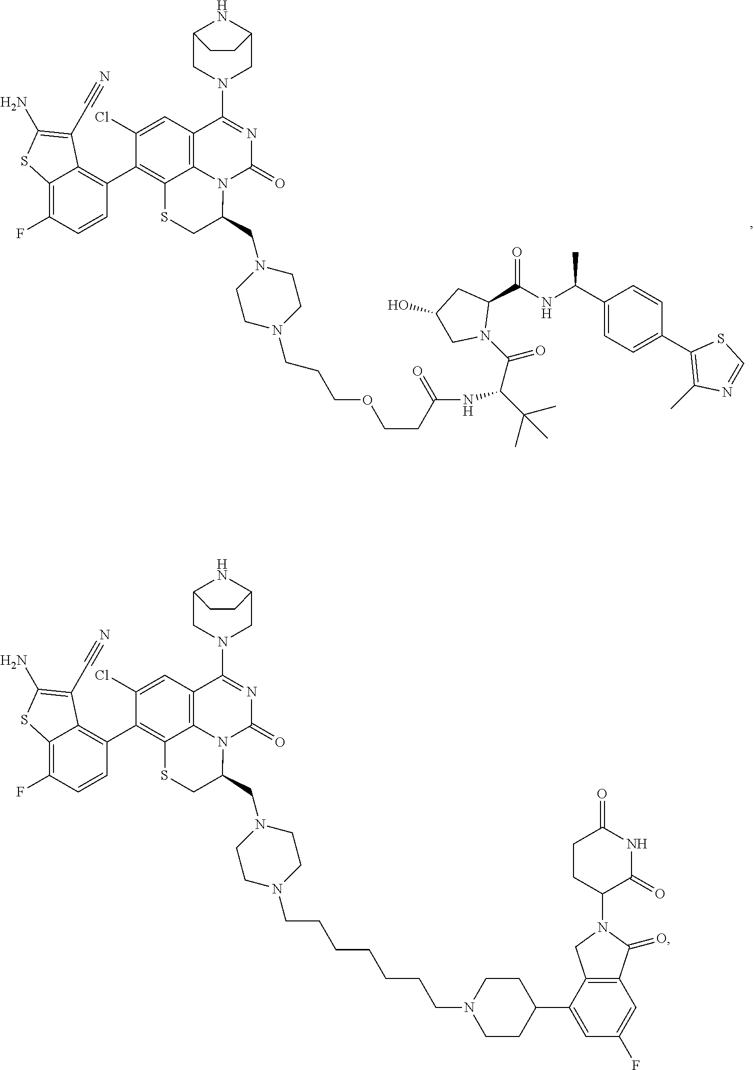 [SMILES: Cc1ncsc1-c1ccc([C@H](C)NC(=O)[C@@H]2C[C@@H](O)CN2C(=O)[C@@H](NC(=O)CCOCCCN2CCN(C[C@H]3CSc4c(-c5ccc(F)c6sc(N)c(C#N)c56)c(Cl)cc5c(N6CC7CCC(C6)N7)nc(=O)n3c45)CC2)C(C)(C)C)cc1 ; N#Cc1c(N)sc2c(F)ccc(-c3c(Cl)cc4c(N5CC6CCC(C5)N6)nc(=O)n5c4c3SC[C@@H]5CN3CCN(CCCCCCCN4CCC(c5cc(F)cc6c5CN(C5CCC(=O)NC5=O)C6=O)CC4)CC3)c12]

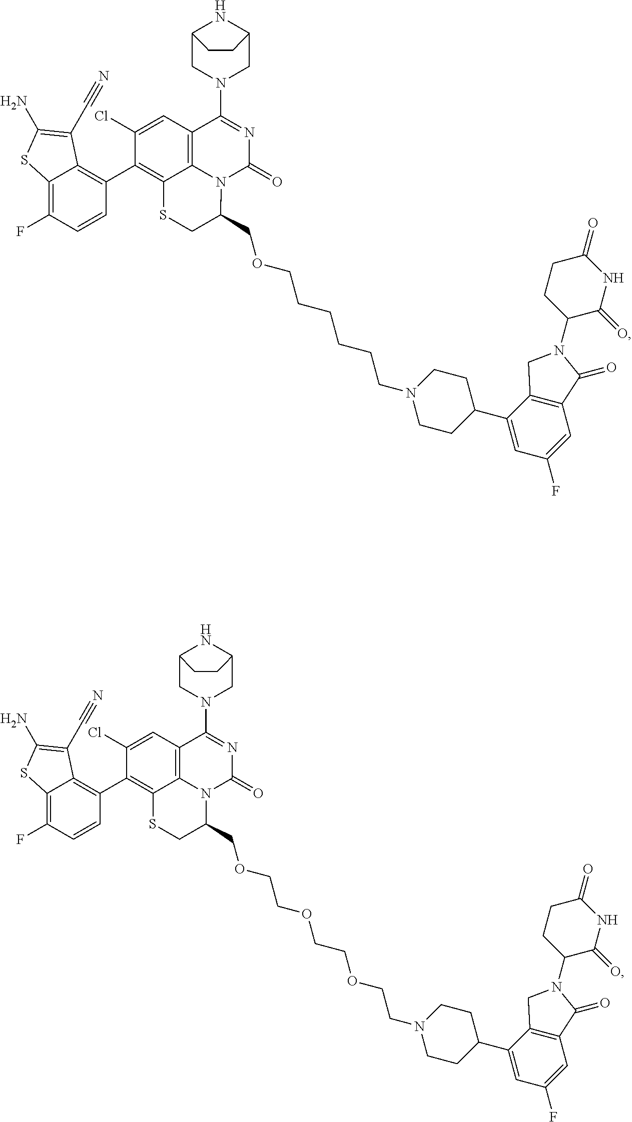 [SMILES: N#Cc1c(N)sc2c(F)ccc(-c3c(Cl)cc4c(N5CC6CCC(C5)N6)nc(=O)n5c4c3SC[C@@H]5COCCCCCCN3CCC(c4cc(F)cc5c4CN(C4CCC(=O)NC4=O)C5=O)CC3)c12 ; N#Cc1c(N)sc2c(F)ccc(-c3c(Cl)cc4c(N5CC6CCC(C5)N6)nc(=O)n5c4c3SC[C@@H]5COCCOCCOCCN3CCC(c4cc(F)cc5c4CN(C4CCC(=O)NC4=O)C5=O)CC3)c12]

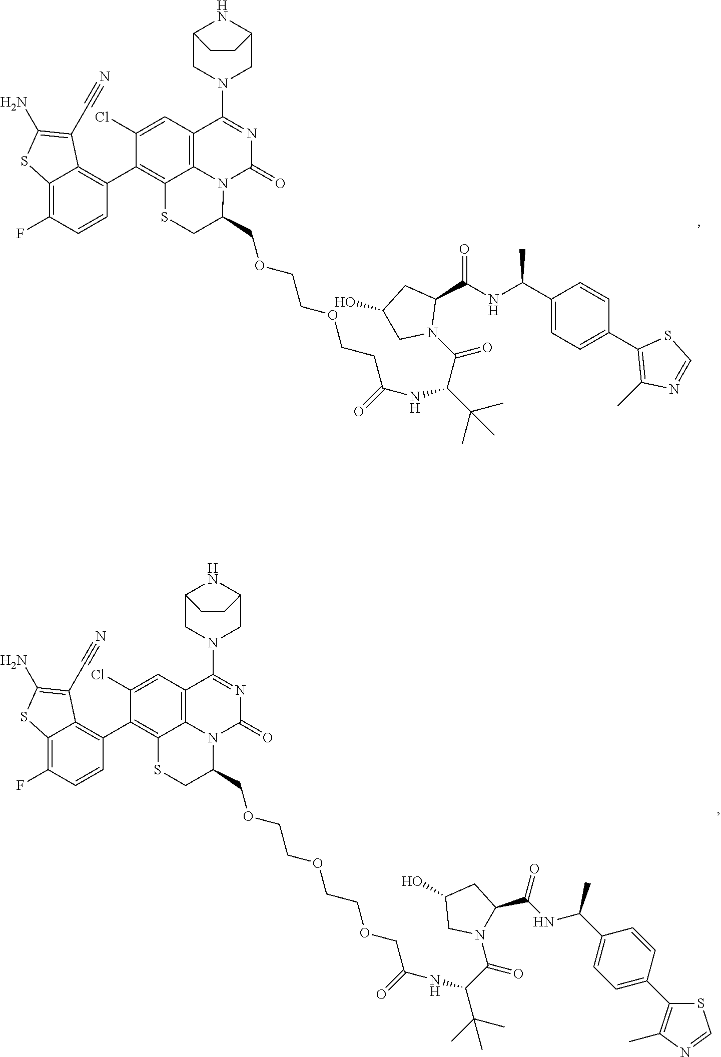 [SMILES: Cc1ncsc1-c1ccc([C@H](C)NC(=O)[C@@H]2C[C@@H](O)CN2C(=O)[C@@H](NC(=O)CCOCCOC[C@H]2CSc3c(-c4ccc(F)c5sc(N)c(C#N)c45)c(Cl)cc4c(N5CC6CCC(C5)N6)nc(=O)n2c34)C(C)(C)C)cc1 ; Cc1ncsc1-c1ccc([C@H](C)NC(=O)[C@@H]2C[C@@H](O)CN2C(=O)[C@@H](NC(=O)COCCOCCOC[C@H]2CSc3c(-c4ccc(F)c5sc(N)c(C#N)c45)c(Cl)cc4c(N5CC6CCC(C5)N6)nc(=O)n2c34)C(C)(C)C)cc1]

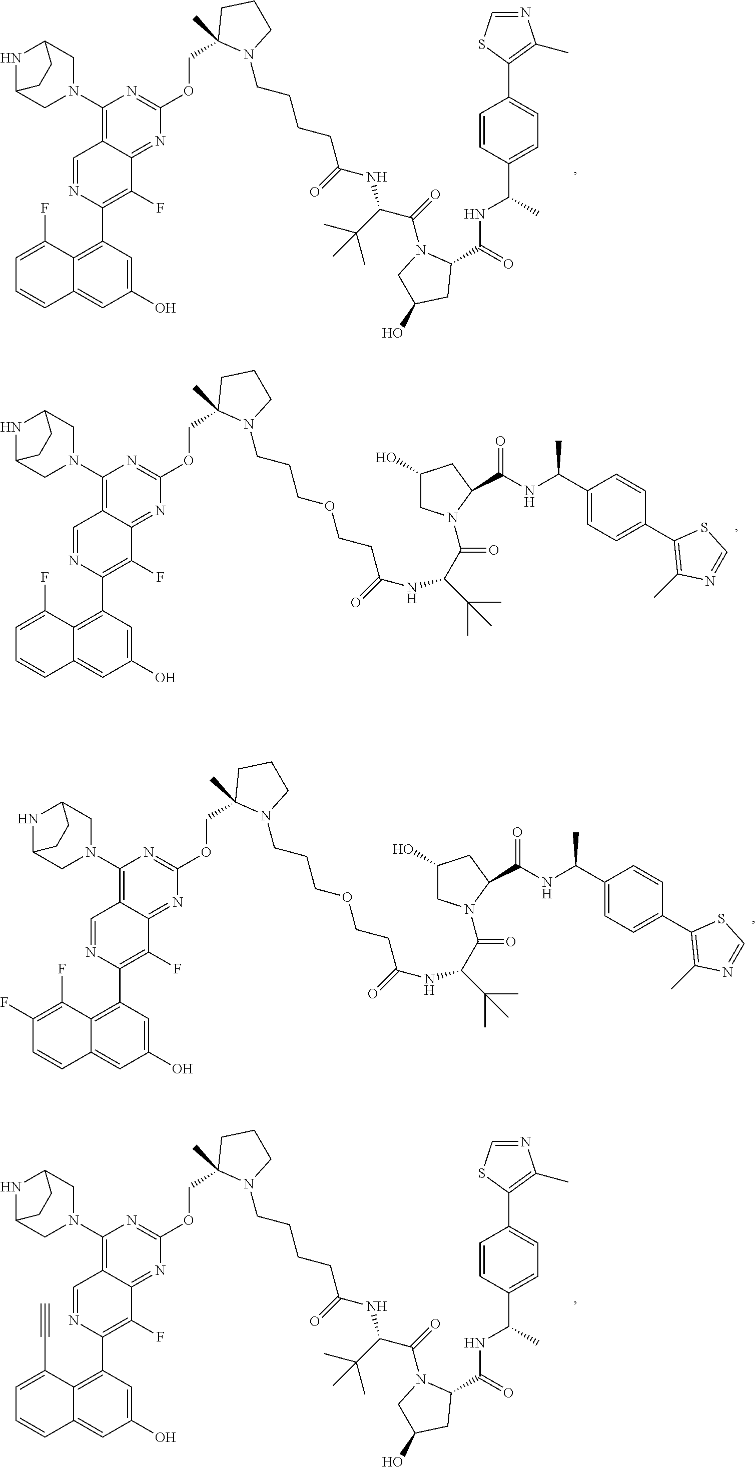

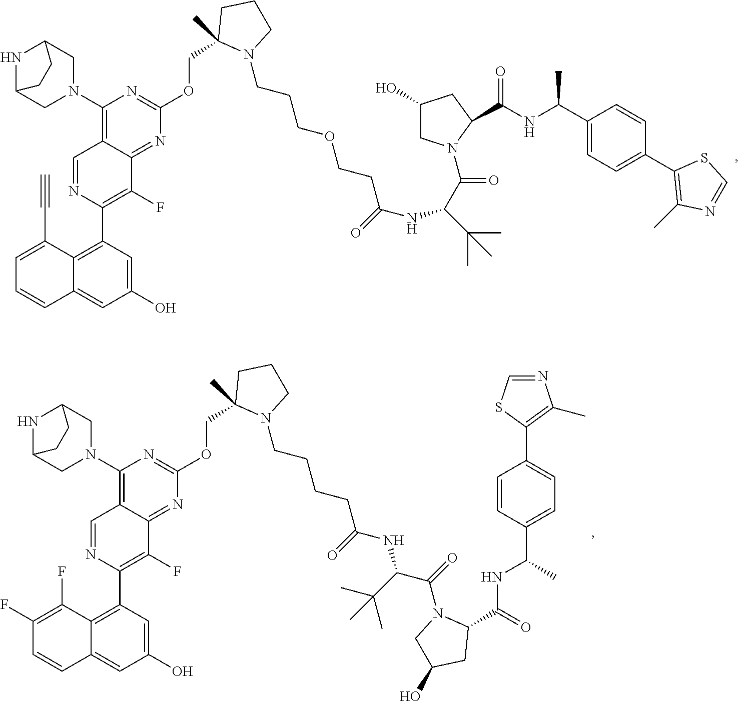 [SMILES: C#Cc1cccc2cc(O)cc(-c3ncc4c(N5CC6CCC(C5)N6)nc(OC[C@]5(C)CCCN5CCCOCCC(=O)N[C@H](C(=O)N5C[C@H](O)C[C@H]5C(=O)N[C@@H](C)c5ccc(-c6scnc6C)cc5)C(C)(C)C)nc4c3F)c12 ; Cc1ncsc1-c1ccc([C@H](C)NC(=O)[C@@H]2C[C@@H](O)CN2C(=O)[C@@H](NC(=O)CCCCN2CCC[C@@]2(C)COc2nc(N3CC4CCC(C3)N4)c3cnc(-c4cc(O)cc5ccc(F)c(F)c45)c(F)c3n2)C(C)(C)C)cc1]

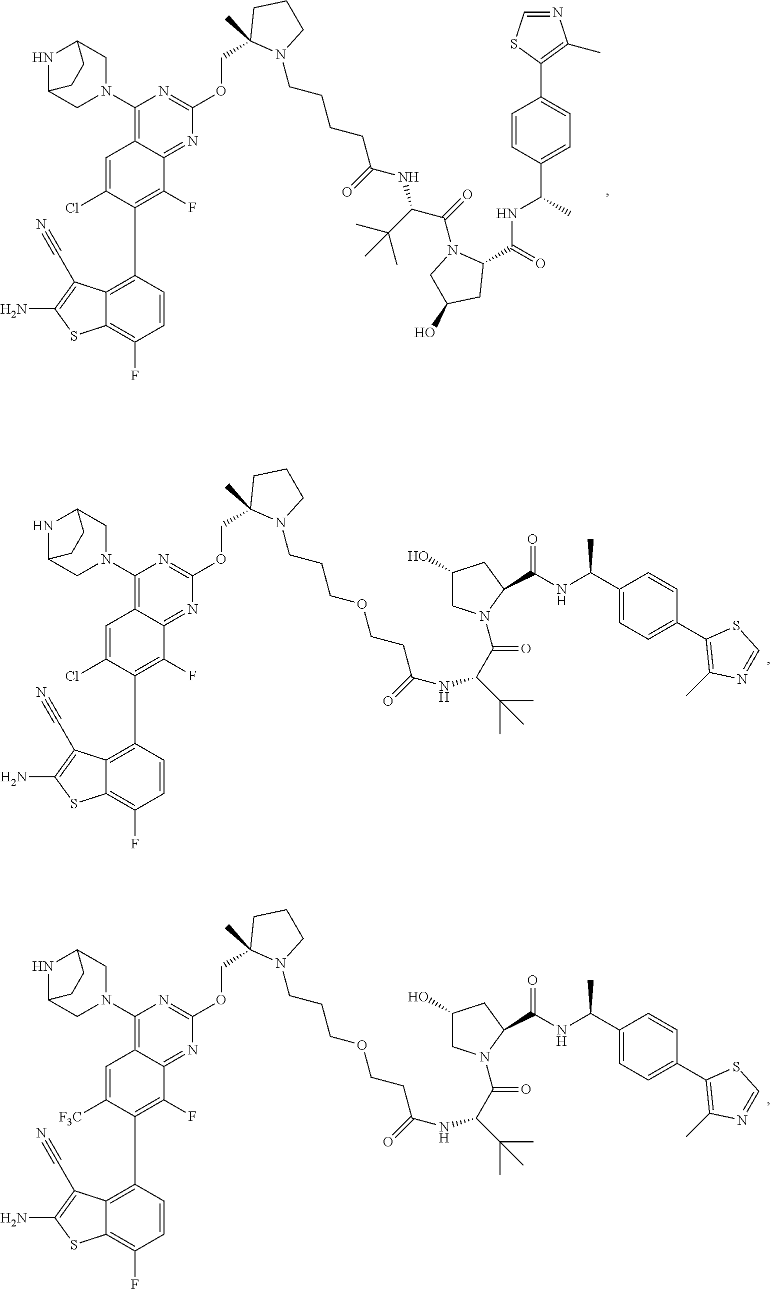

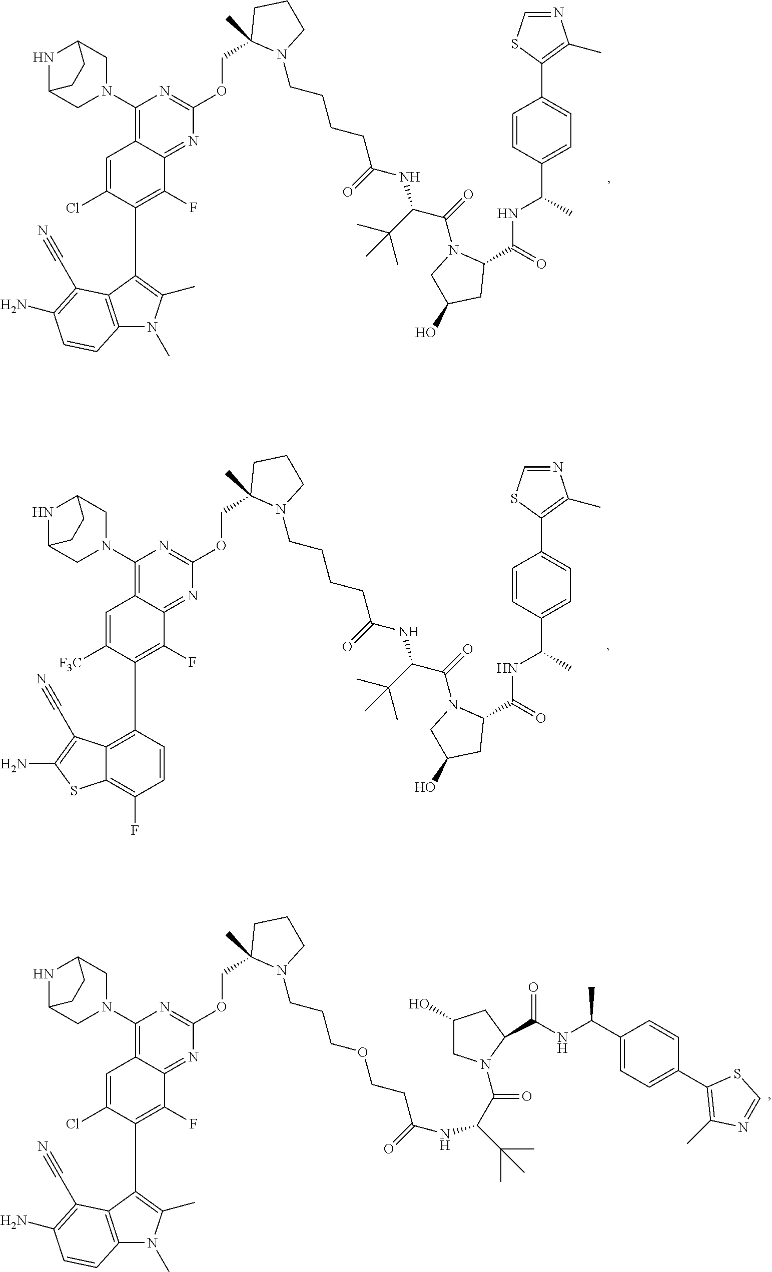

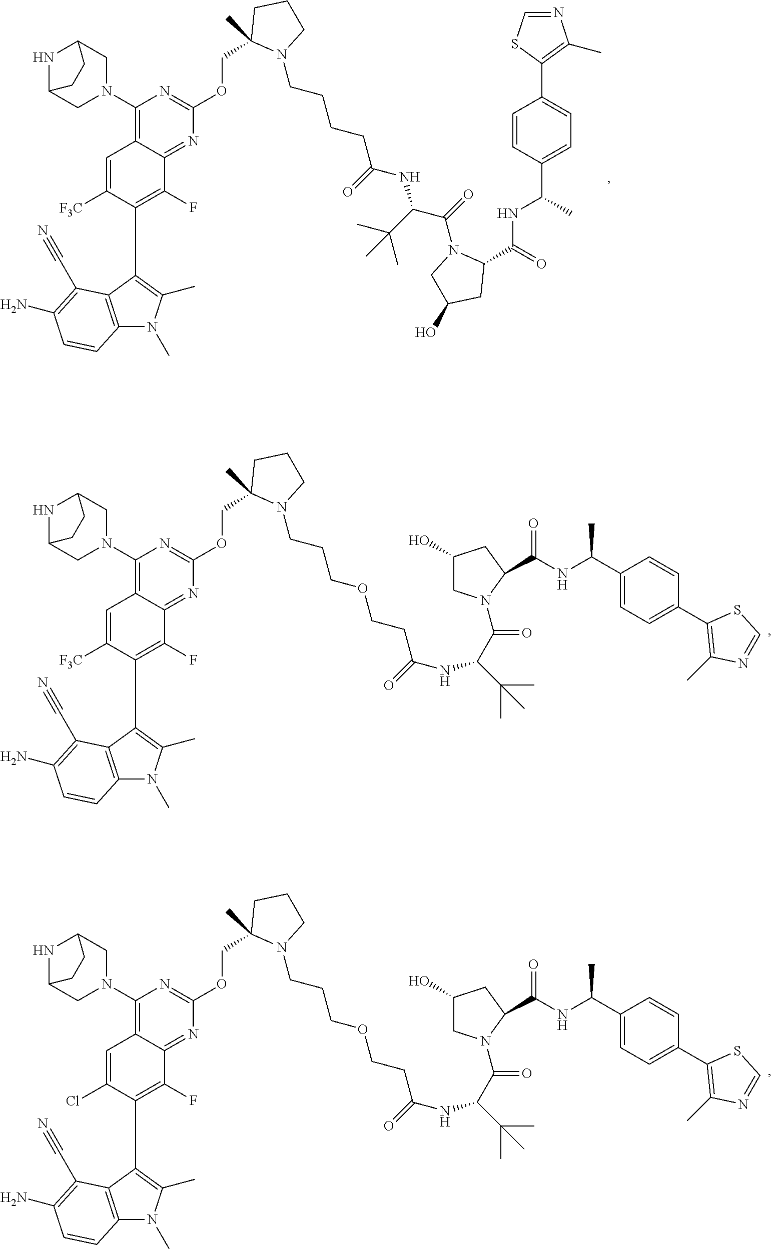

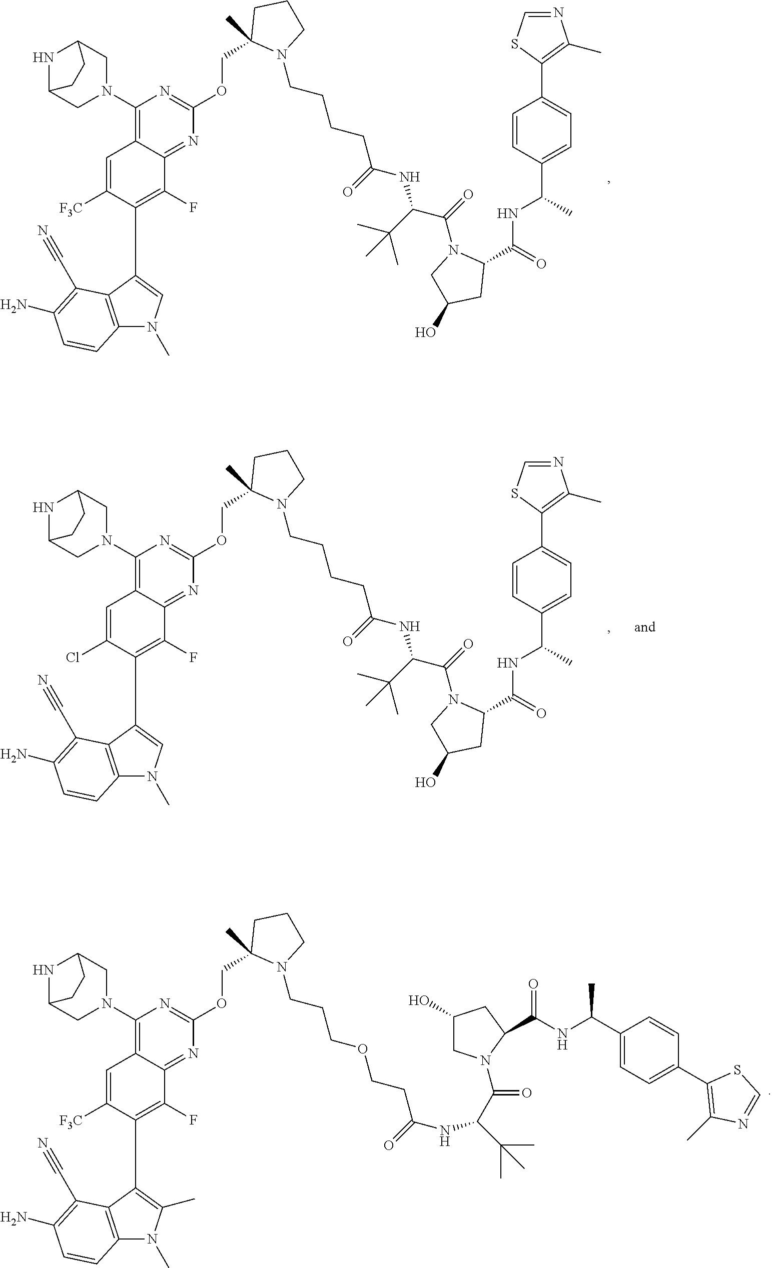

In some embodiments, the conjugate is selected from:

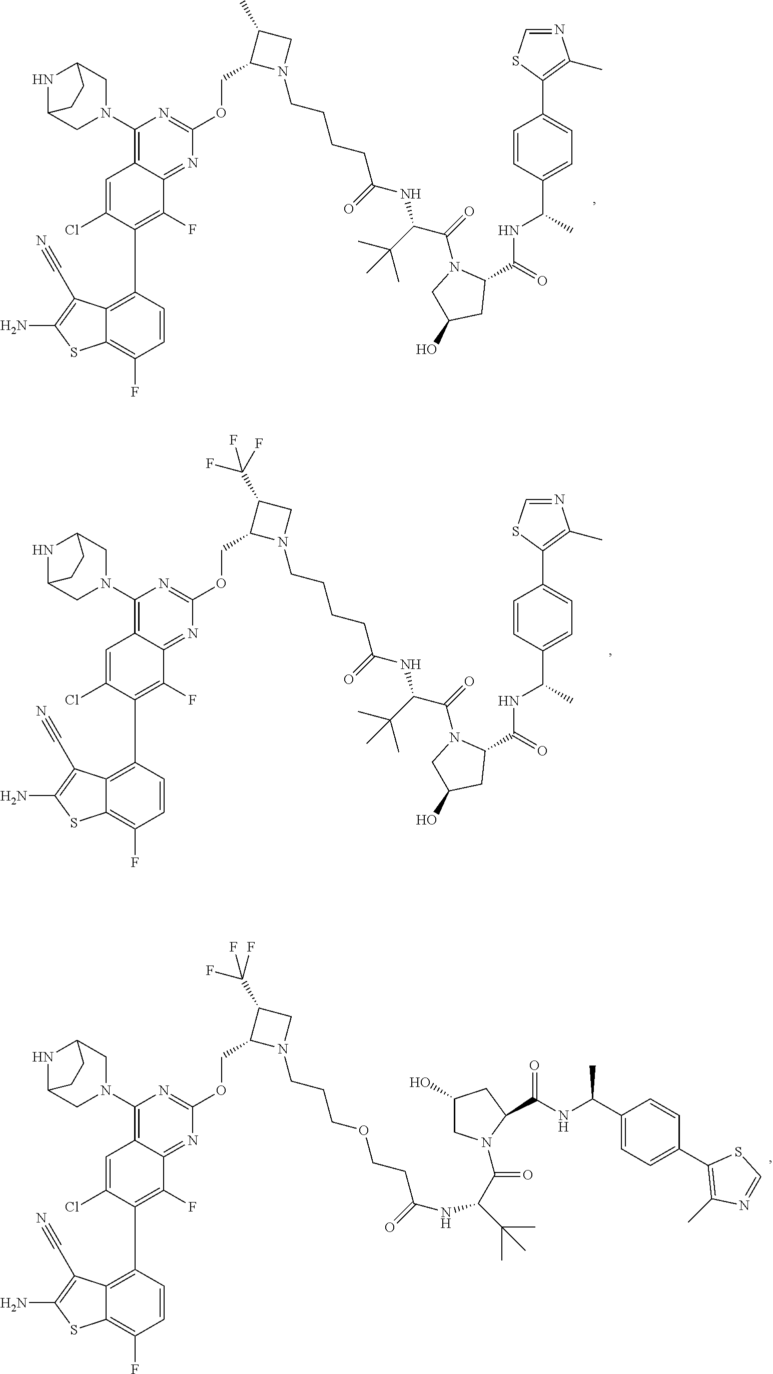

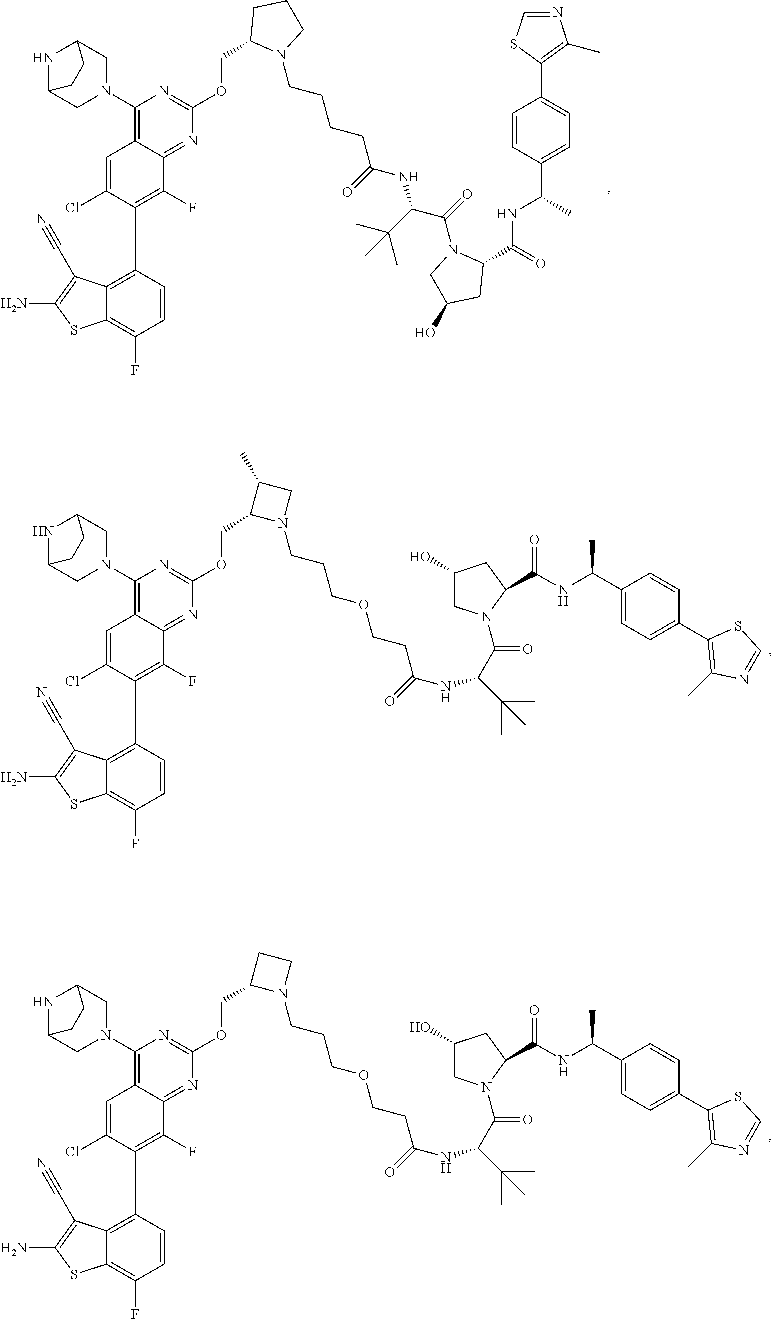 [SMILES: Cc1ncsc1-c1ccc([C@H](C)NC(=O)[C@@H]2C[C@@H](O)CN2C(=O)[C@@H](NC(=O)CCCCN2CCC[C@H]2COc2nc(N3CC4CCC(C3)N4)c3cc(Cl)c(-c4ccc(F)c5sc(N)c(C#N)c45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2C[C@@H](C)[C@H]2COc2nc(N3CC4CCC(C3)N4)c3cc(Cl)c(-c4ccc(F)c5sc(N)c(C#N)c45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2CC[C@H]2COc2nc(N3CC4CCC(C3)N4)c3cc(Cl)c(-c4ccc(F)c5sc(N)c(C#N)c45)c(F)c3n2)C(C)(C)C)cc1]

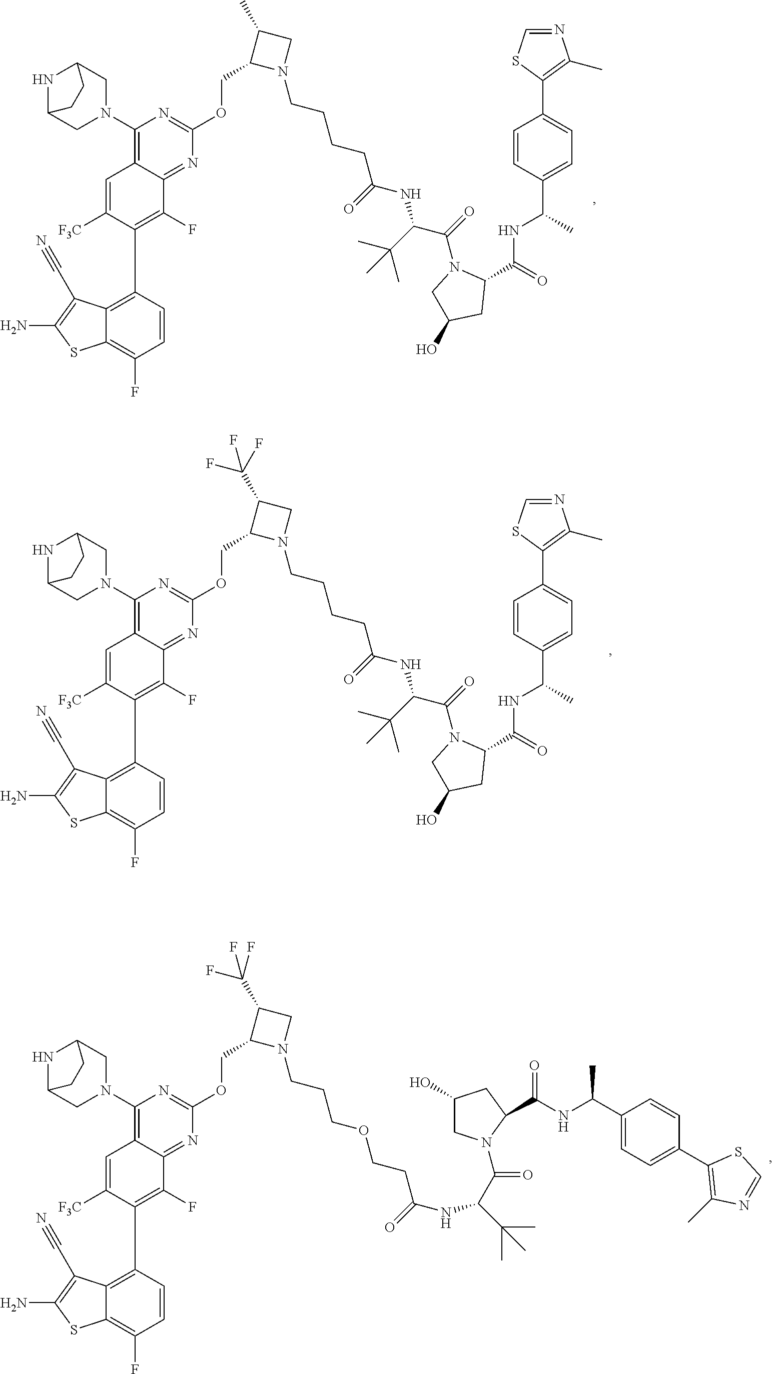

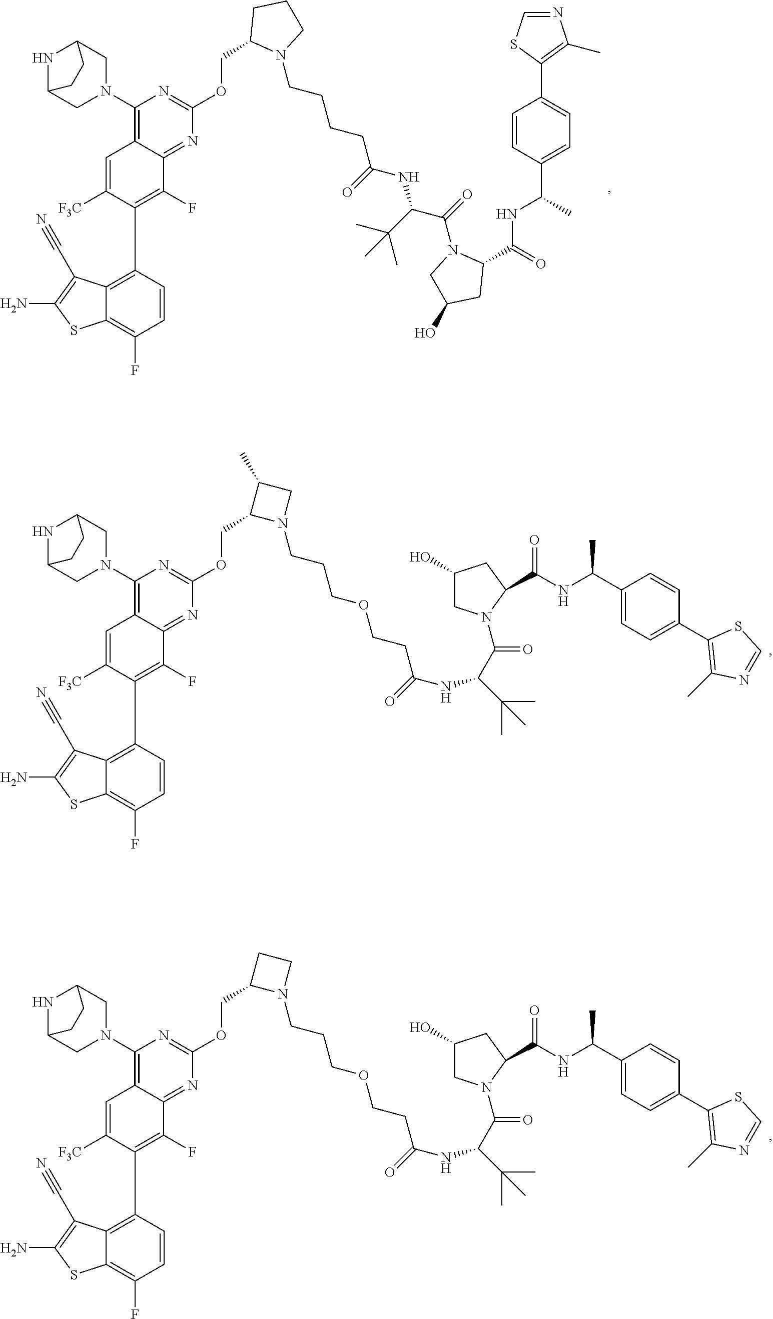

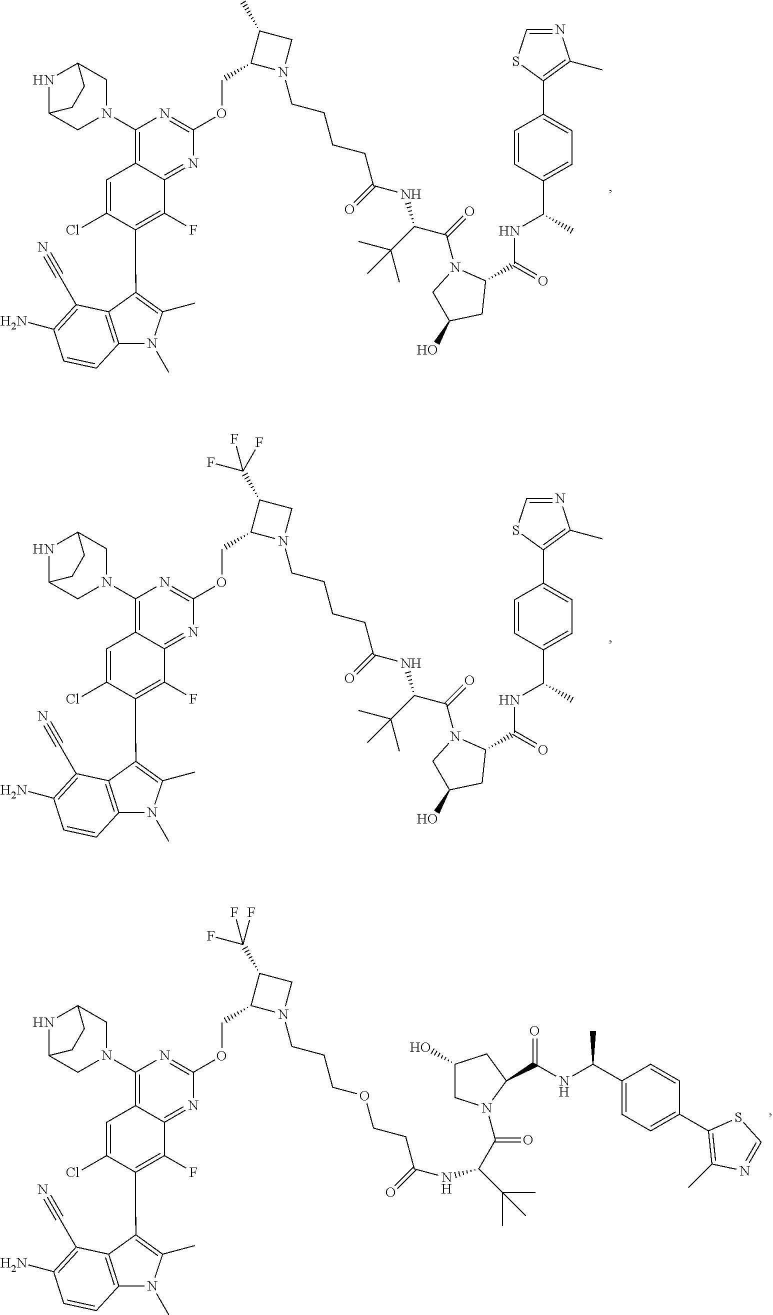

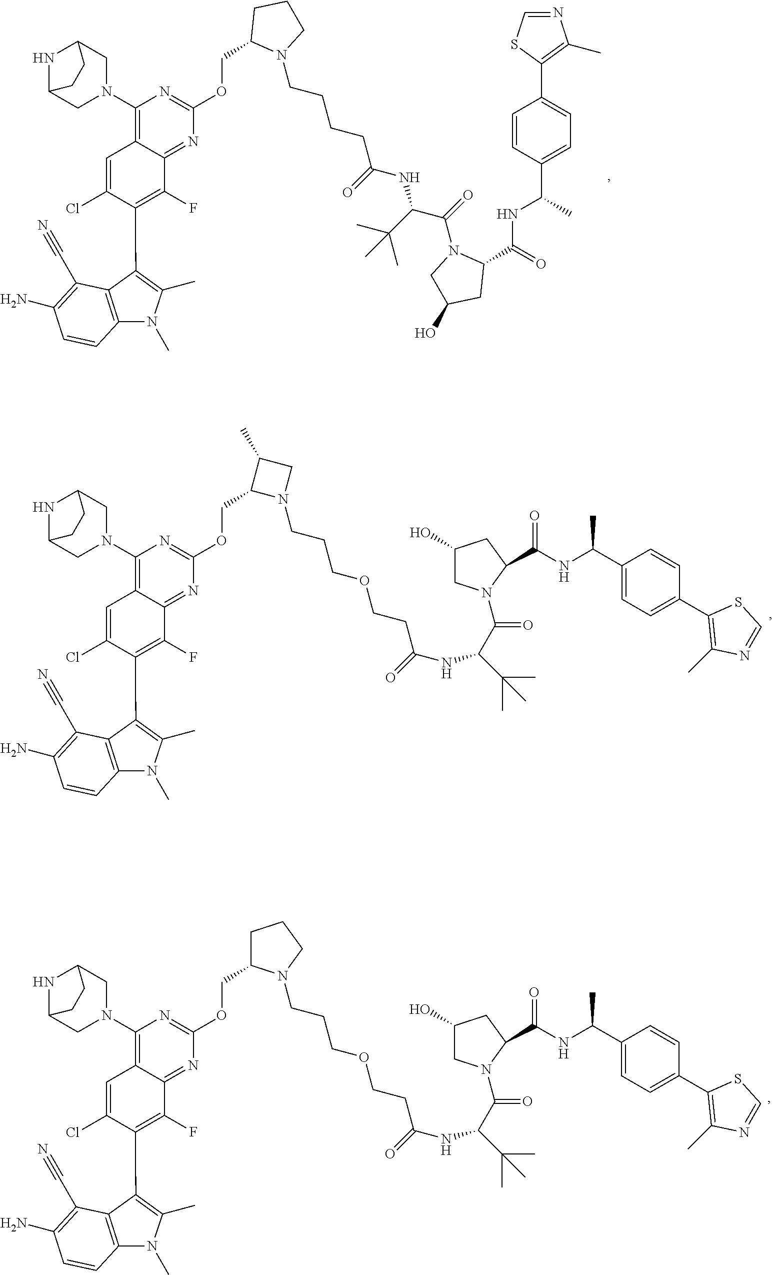

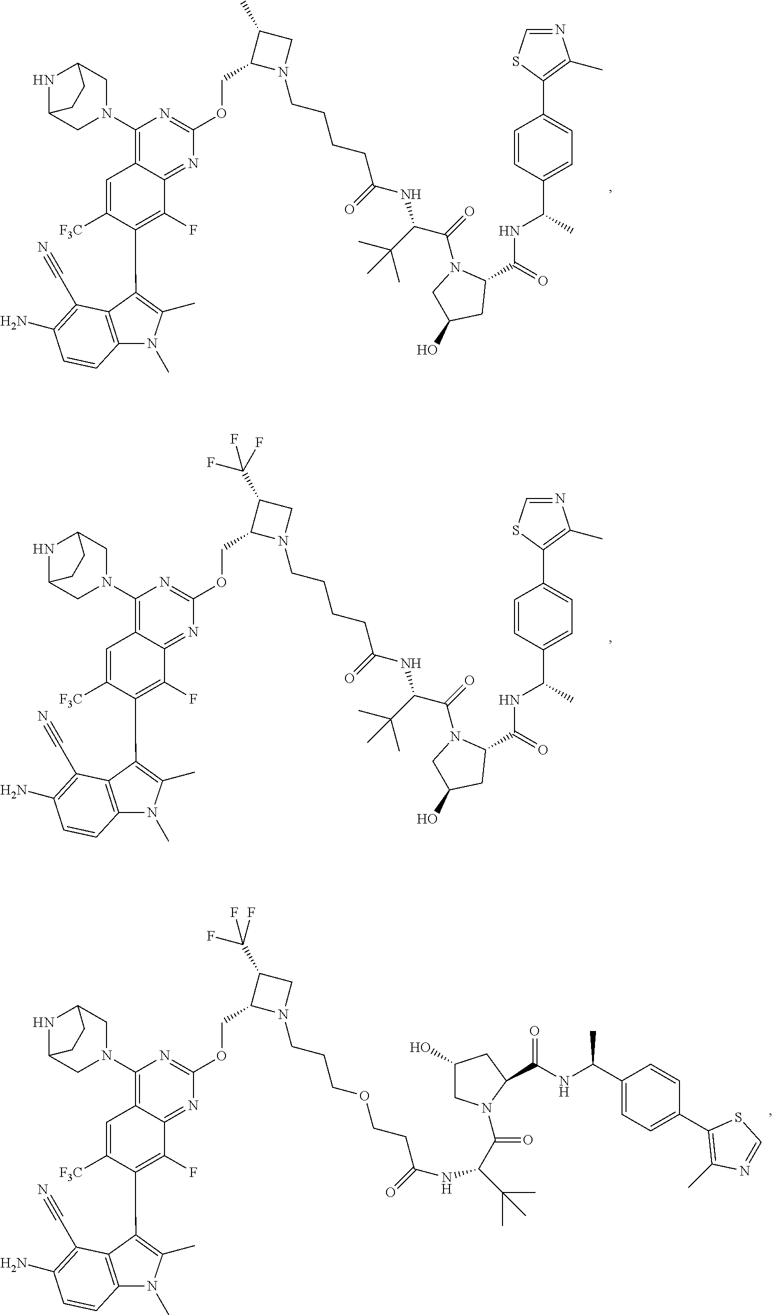

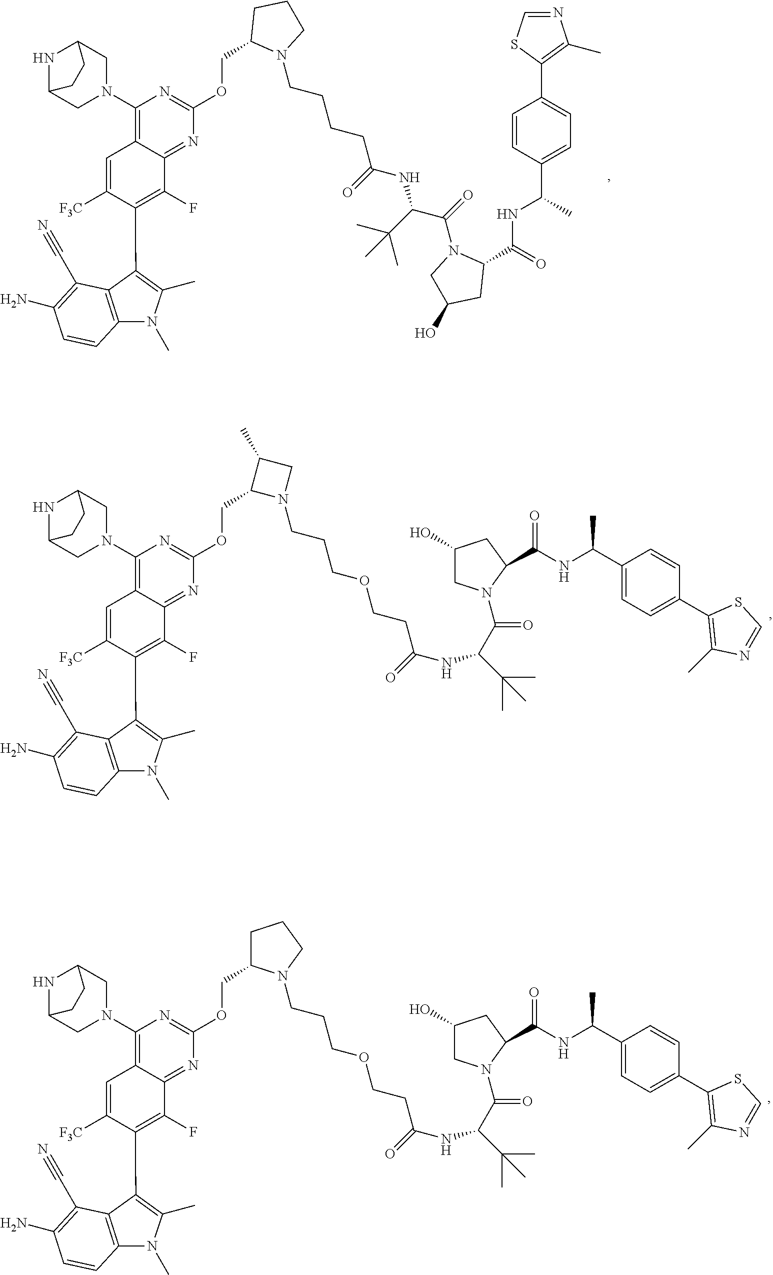

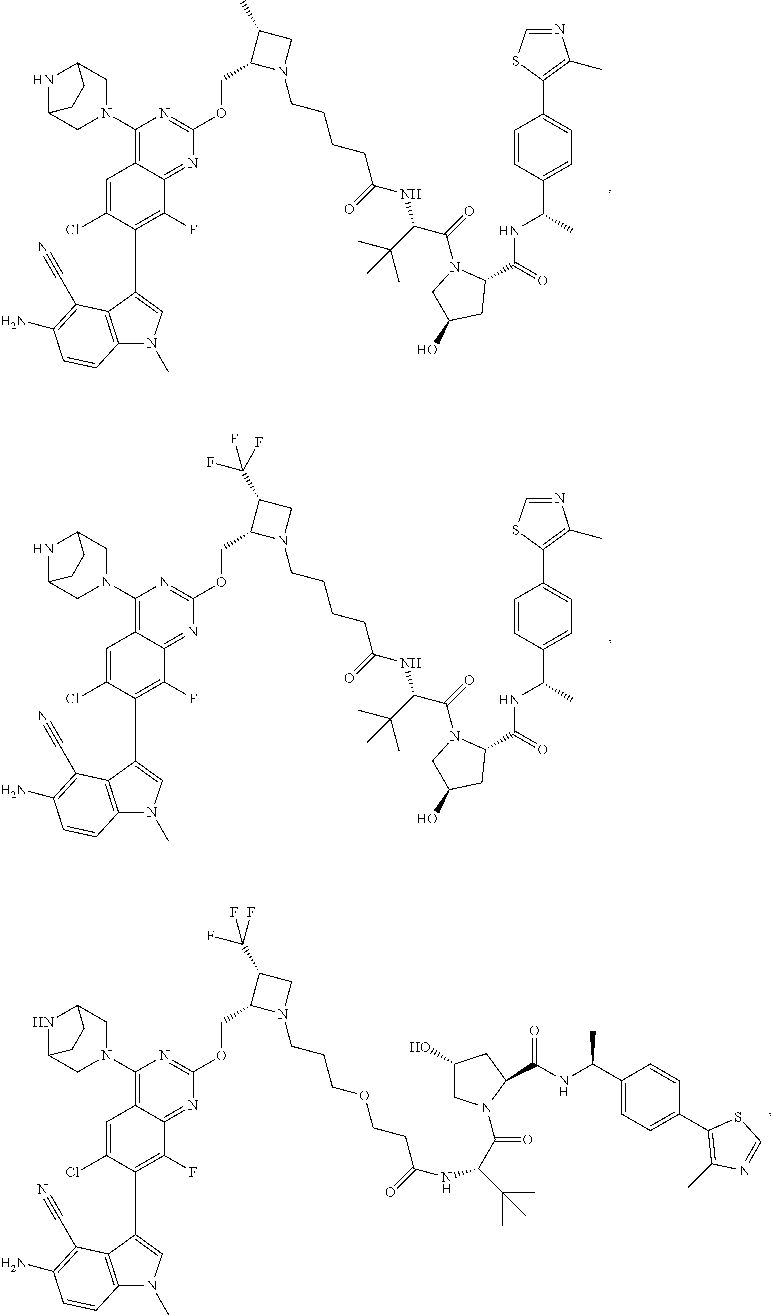

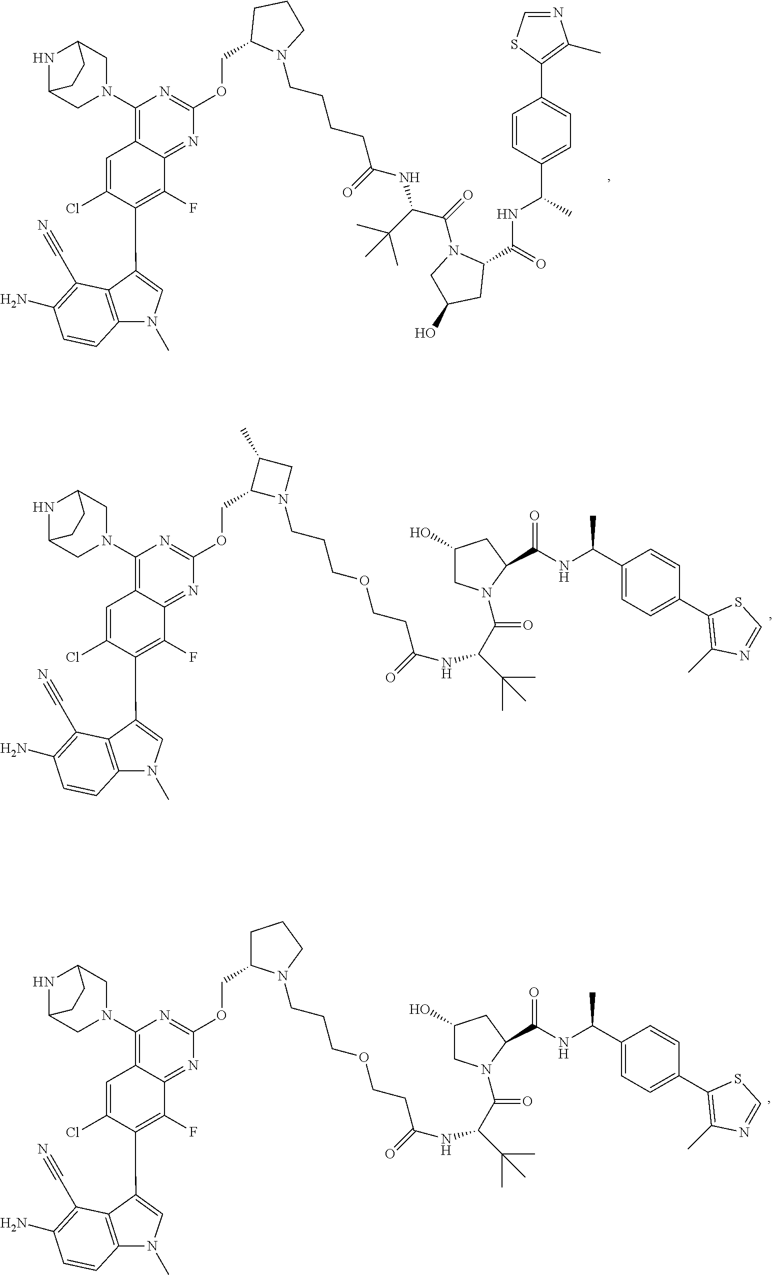 [SMILES: Cc1ncsc1-c1ccc([C@H](C)NC(=O)[C@@H]2C[C@@H](O)CN2C(=O)[C@@H](NC(=O)CCCCN2CCC[C@H]2COc2nc(N3CC4CCC(C3)N4)c3cc(Cl)c(-c4cn(C)c5ccc(N)c(C#N)c45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2C[C@@H](C)[C@H]2COc2nc(N3CC4CCC(C3)N4)c3cc(Cl)c(-c4cn(C)c5ccc(N)c(C#N)c45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cc(Cl)c(-c4cn(C)c5ccc(N)c(C#N)c45)c(F)c3n2)C(C)(C)C)cc1]

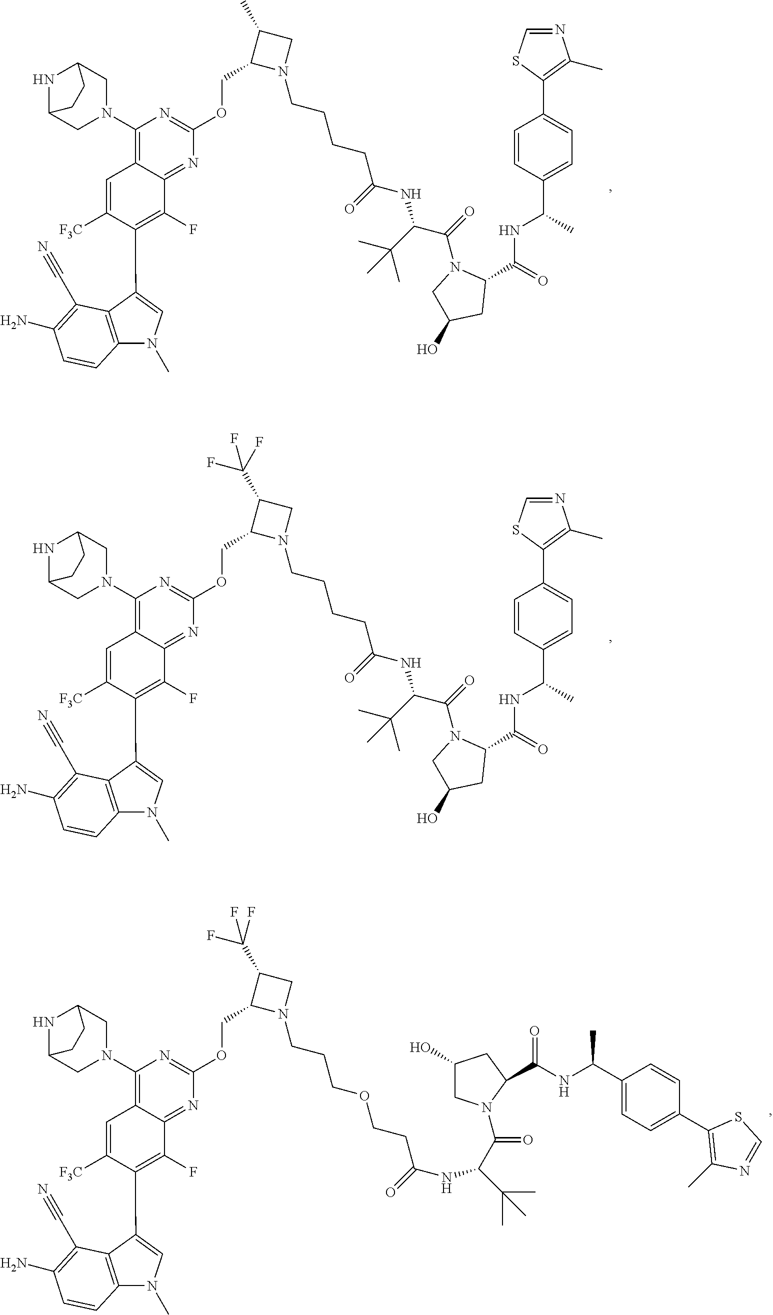

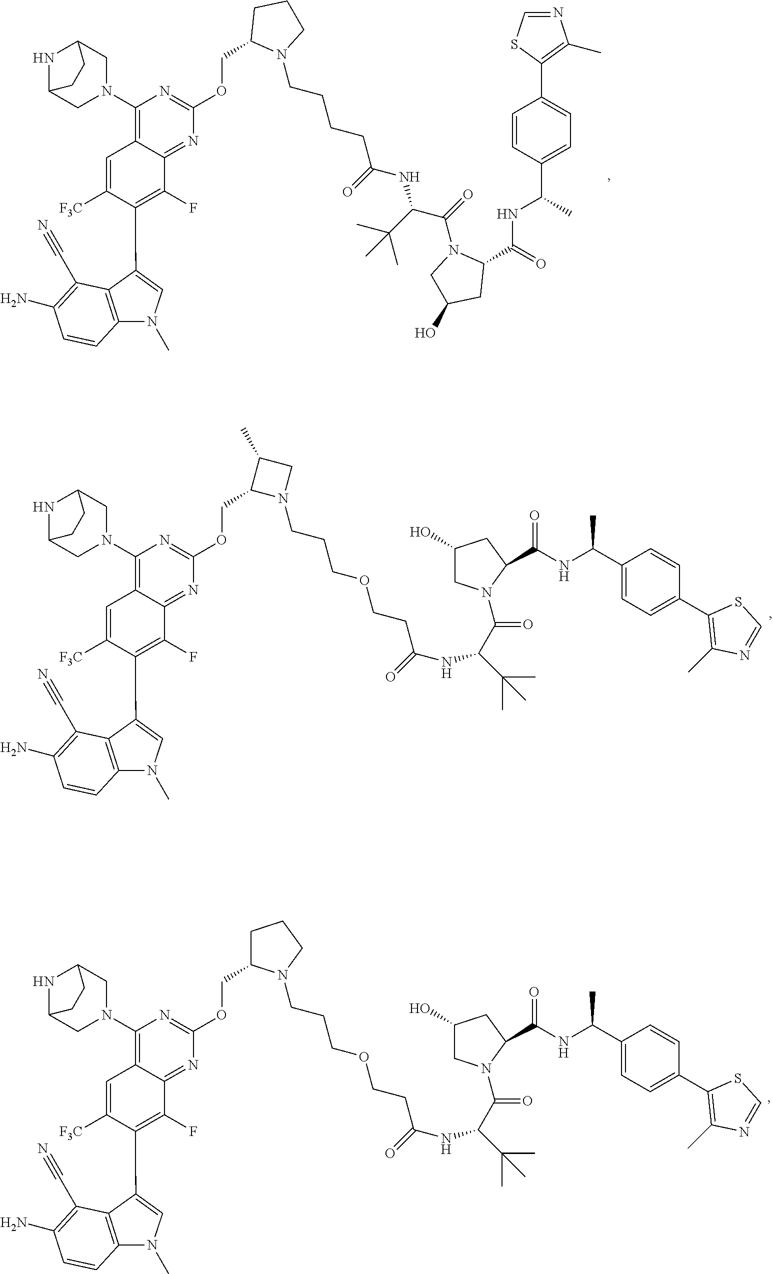

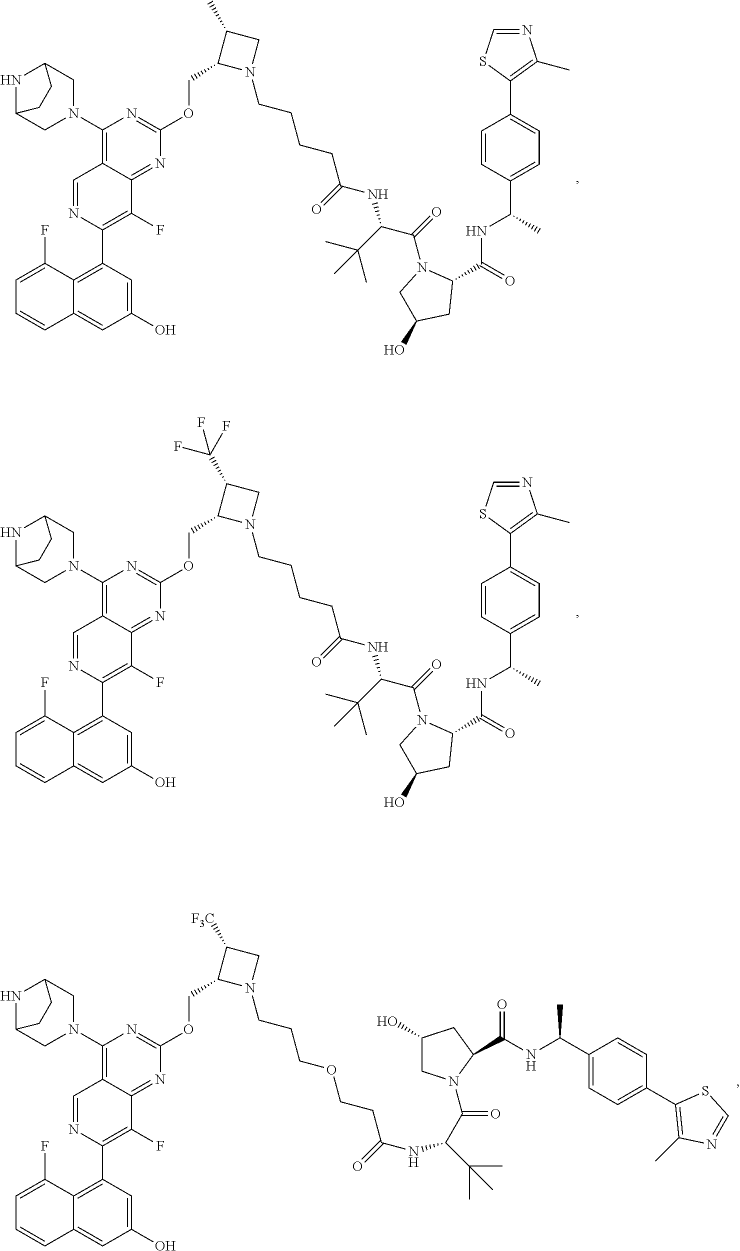

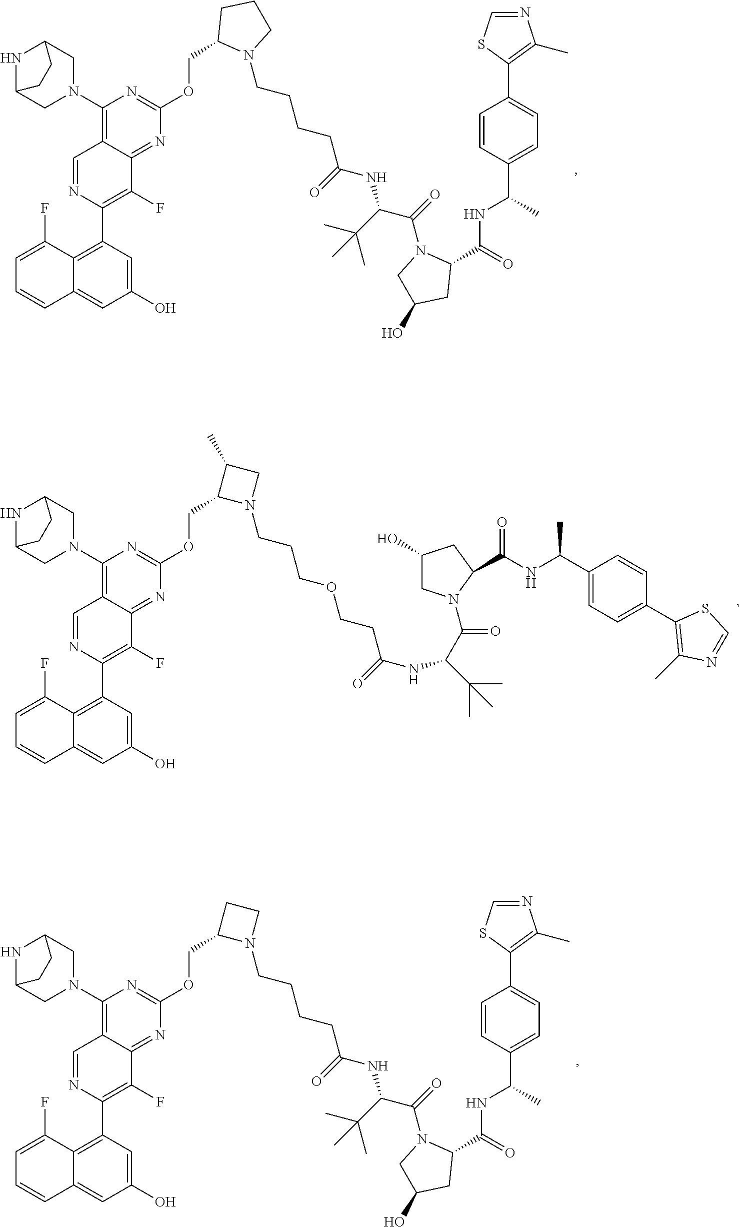 [SMILES: Cc1ncsc1-c1ccc([C@H](C)NC(=O)[C@@H]2C[C@@H](O)CN2C(=O)[C@@H](NC(=O)CCCCN2CCC[C@H]2COc2nc(N3CC4CCC(C3)N4)c3cnc(-c4cc(O)cc5cccc(F)c45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2C[C@@H](C)[C@H]2COc2nc(N3CC4CCC(C3)N4)c3cnc(-c4cc(O)cc5cccc(F)c45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCCCN2CC[C@H]2COc2nc(N3CC4CCC(C3)N4)c3cnc(-c4cc(O)cc5cccc(F)c45)c(F)c3n2)C(C)(C)C)cc1]

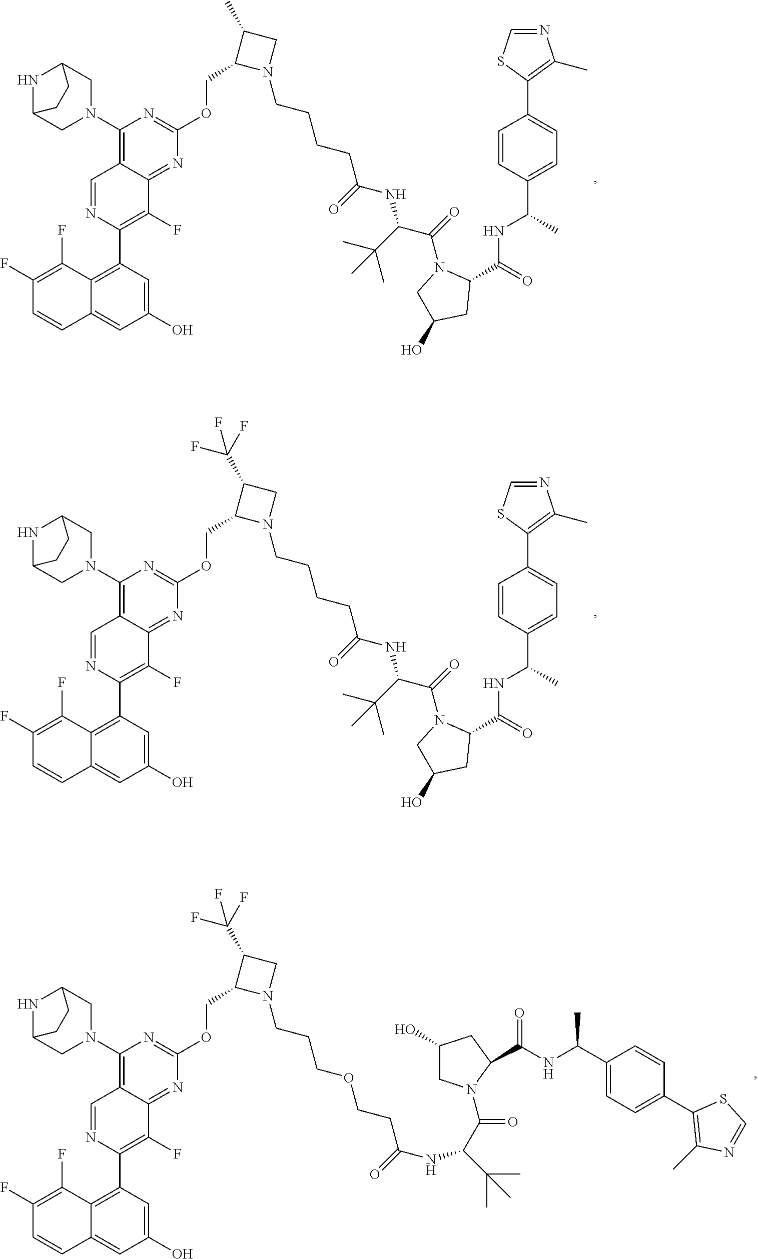

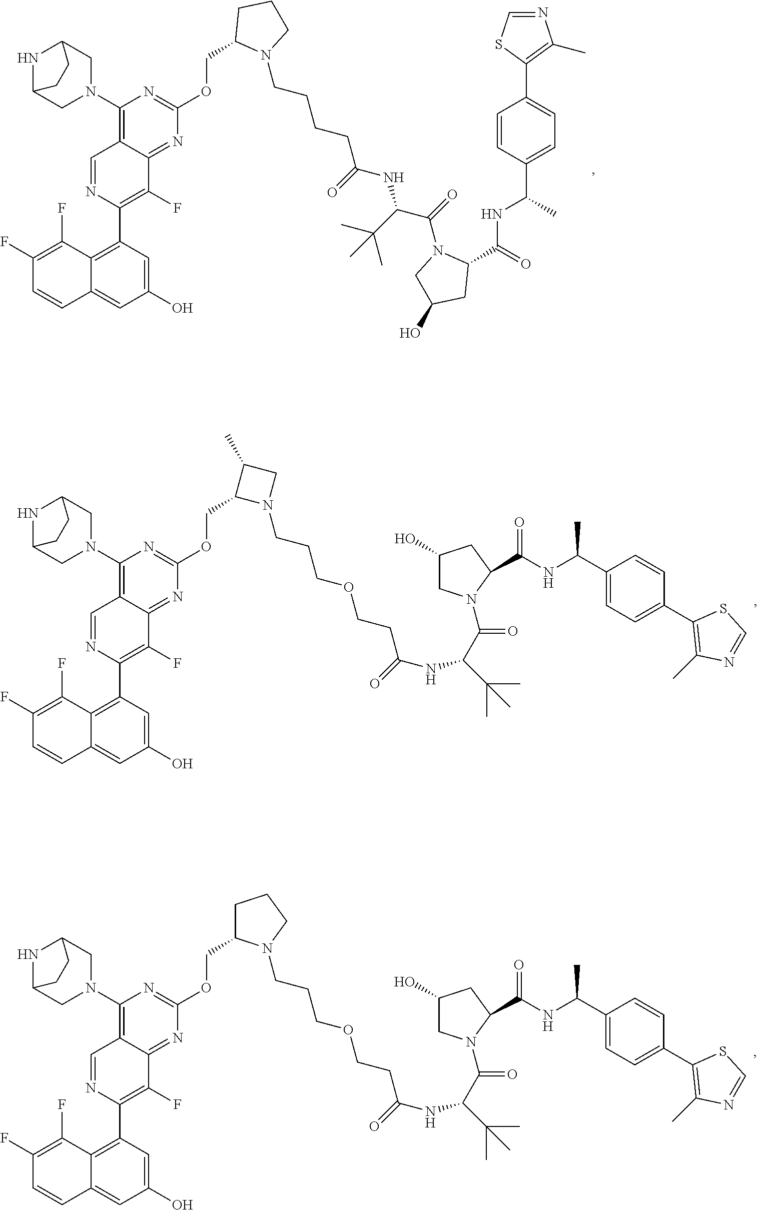 [SMILES: Cc1ncsc1-c1ccc([C@H](C)NC(=O)[C@@H]2C[C@@H](O)CN2C(=O)[C@@H](NC(=O)CCCCN2CCC[C@H]2COc2nc(N3CC4CCC(C3)N4)c3cnc(-c4cc(O)cc5ccc(F)c(F)c45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2C[C@@H](C)[C@H]2COc2nc(N3CC4CCC(C3)N4)c3cnc(-c4cc(O)cc5ccc(F)c(F)c45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cnc(-c4cc(O)cc5ccc(F)c(F)c45)c(F)c3n2)C(C)(C)C)cc1]

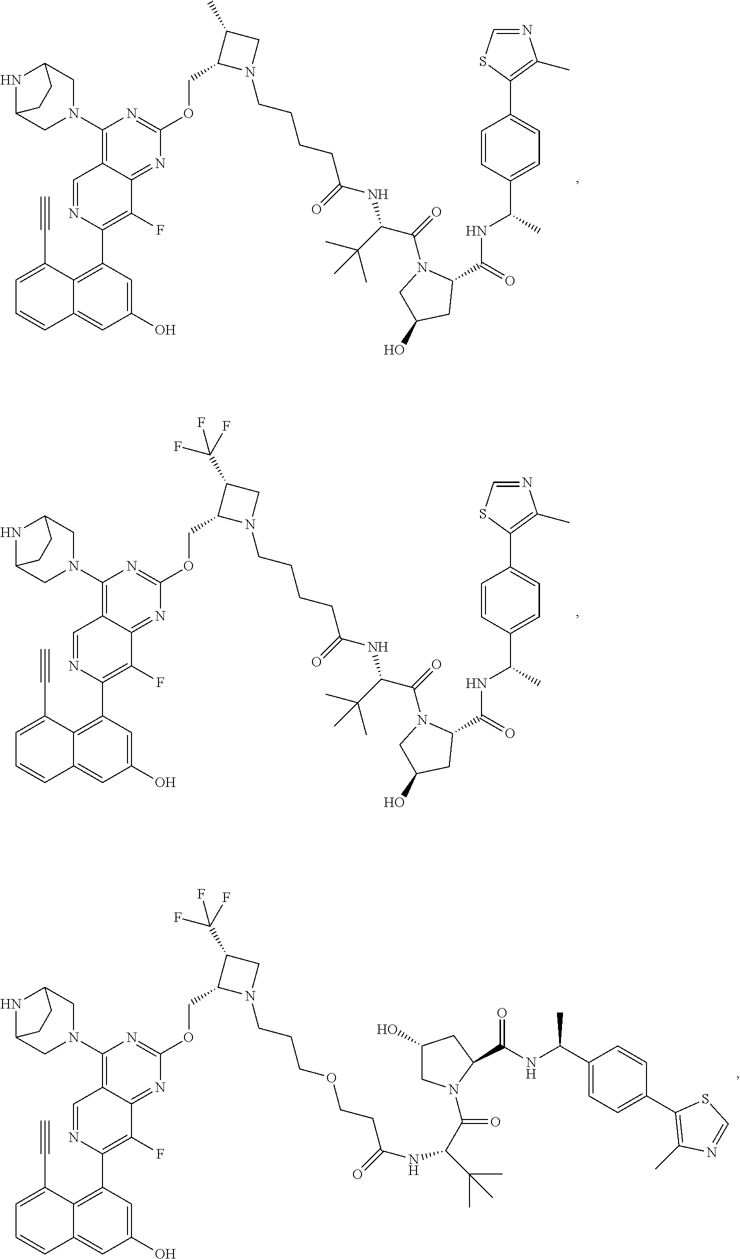 [SMILES: C#Cc1cccc2cc(O)cc(-c3ncc4c(N5CC6CCC(C5)N6)nc(OC[C@@H]5[C@H](C)CN5CCCCC(=O)N[C@H](C(=O)N5C[C@H](O)C[C@H]5C(=O)N[C@@H](C)c5ccc(-c6scnc6C)cc5)C(C)(C)C)nc4c3F)c12 ; C#Cc1cccc2cc(O)cc(-c3ncc4c(N5CC6CCC(C5)N6)nc(OC[C@@H]5[C@H](C(F)(F)F)CN5CCCCC(=O)N[C@H](C(=O)N5C[C@H](O)C[C@H]5C(=O)N[C@@H](C)c5ccc(-c6scnc6C)cc5)C(C)(C)C)nc4c3F)c12 ; C#Cc1cccc2cc(O)cc(-c3ncc4c(N5CC6CCC(C5)N6)nc(OC[C@@H]5[C@H](C(F)(F)F)CN5CCCOCCC(=O)N[C@H](C(=O)N5C[C@H](O)C[C@H]5C(=O)N[C@@H](C)c5ccc(-c6scnc6C)cc5)C(C)(C)C)nc4c3F)c12]

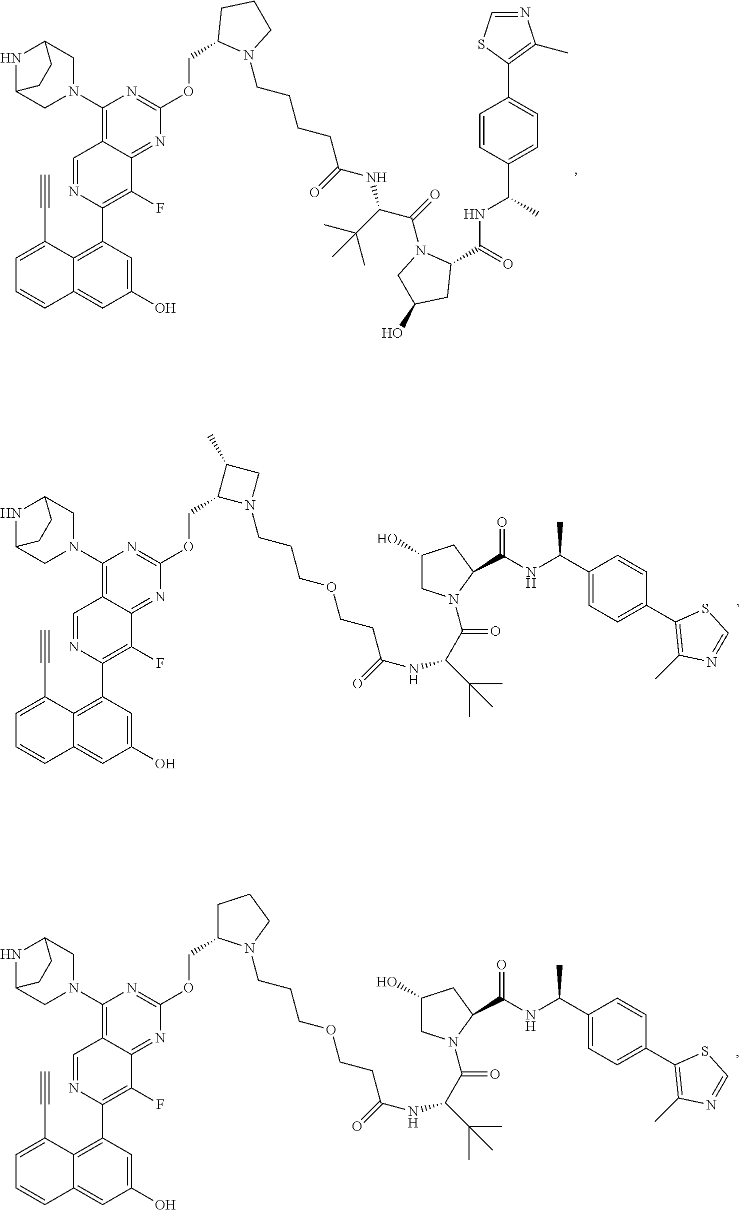 [SMILES: C#Cc1cccc2cc(O)cc(-c3ncc4c(N5CC6CCC(C5)N6)nc(OC[C@@H]5CCCN5CCCCC(=O)N[C@H](C(=O)N5C[C@H](O)C[C@H]5C(=O)N[C@@H](C)c5ccc(-c6scnc6C)cc5)C(C)(C)C)nc4c3F)c12 ; C#Cc1cccc2cc(O)cc(-c3ncc4c(N5CC6CCC(C5)N6)nc(OC[C@@H]5[C@H](C)CN5CCCOCCC(=O)N[C@H](C(=O)N5C[C@H](O)C[C@H]5C(=O)N[C@@H](C)c5ccc(-c6scnc6C)cc5)C(C)(C)C)nc4c3F)c12 ; C#Cc1cccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6scnc6C)cc5)C(C)(C)C)nc4c3F)c12]

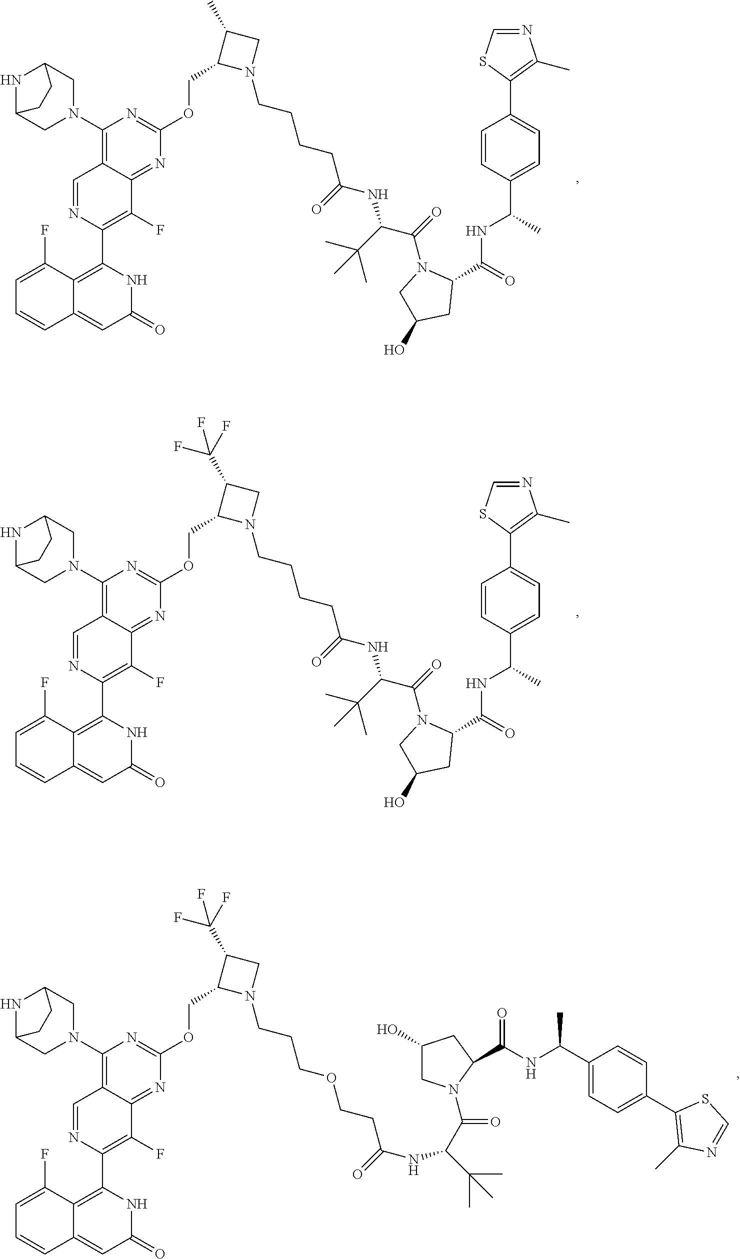

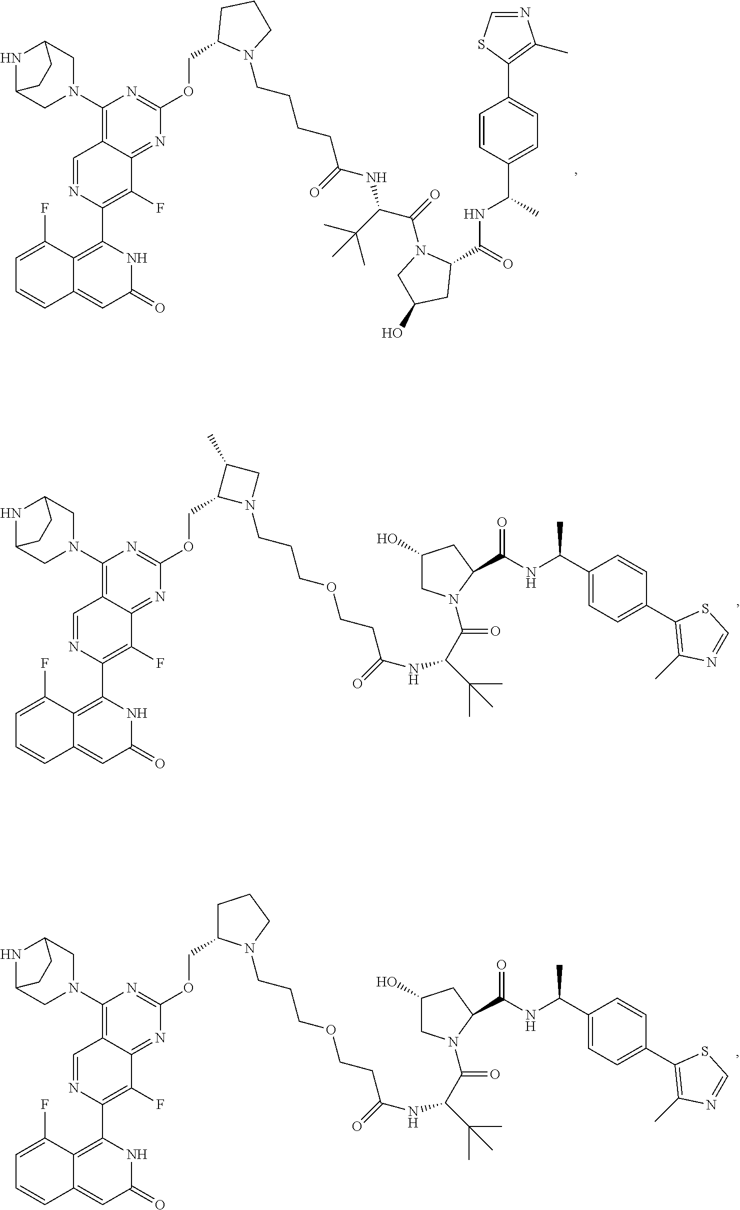 [SMILES: Cc1ncsc1-c1ccc([C@H](C)NC(=O)[C@@H]2C[C@@H](O)CN2C(=O)[C@@H](NC(=O)CCCCN2CCC[C@H]2COc2nc(N3CC4CCC(C3)N4)c3cnc(-c4[nH]c(=O)cc5cccc(F)c45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2C[C@@H](C)[C@H]2COc2nc(N3CC4CCC(C3)N4)c3cnc(-c4[nH]c(=O)cc5cccc(F)c45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cnc(-c4[nH]c(=O)cc5cccc(F)c45)c(F)c3n2)C(C)(C)C)cc1]

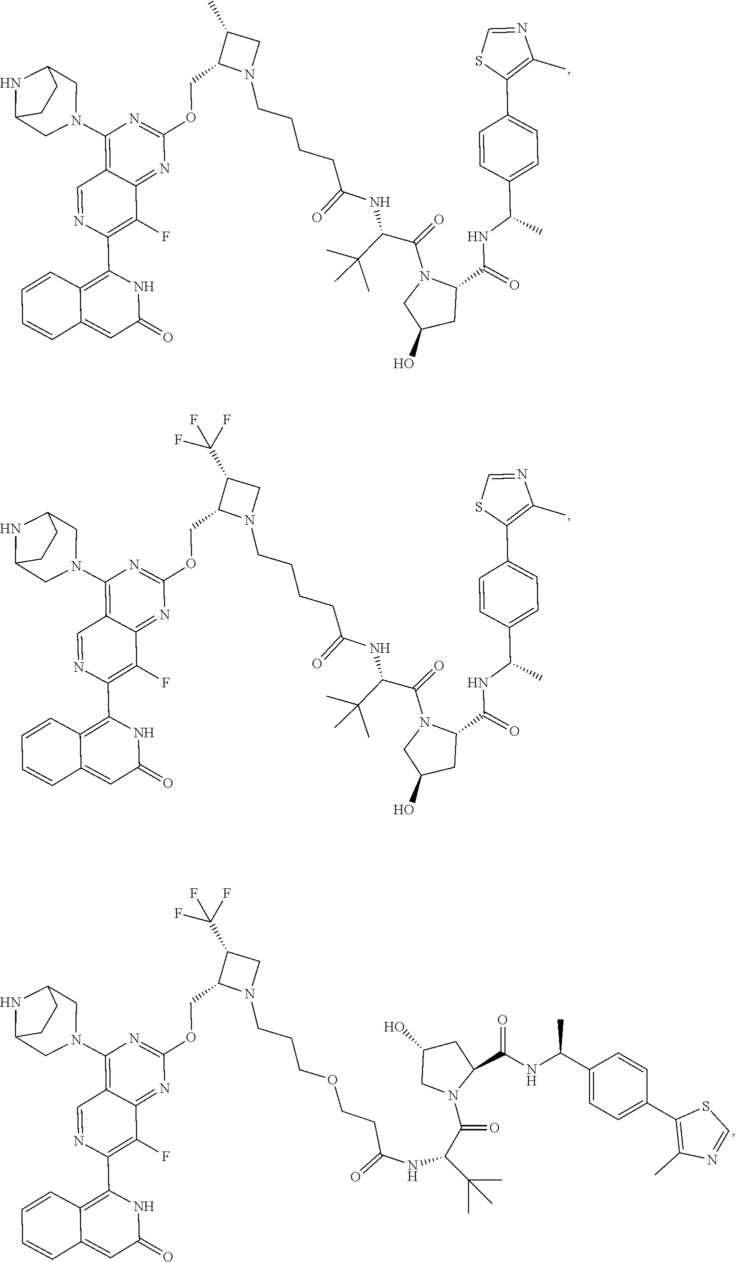

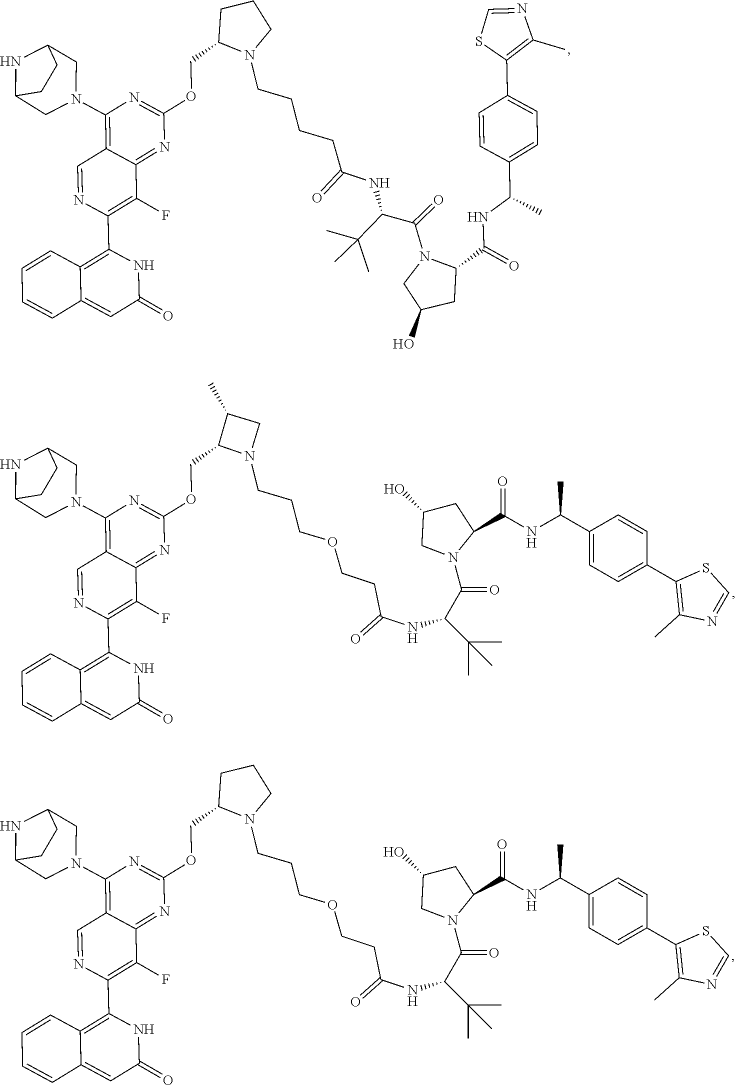 [SMILES: Cc1ncsc1-c1ccc([C@H](C)NC(=O)[C@@H]2C[C@@H](O)CN2C(=O)[C@@H](NC(=O)CCCCN2CCC[C@H]2COc2nc(N3CC4CCC(C3)N4)c3cnc(-c4[nH]c(=O)cc5ccccc45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2C[C@@H](C)[C@H]2COc2nc(N3CC4CCC(C3)N4)c3cnc(-c4[nH]c(=O)cc5ccccc45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cnc(-c4[nH]c(=O)cc5ccccc45)c(F)c3n2)C(C)(C)C)cc1]

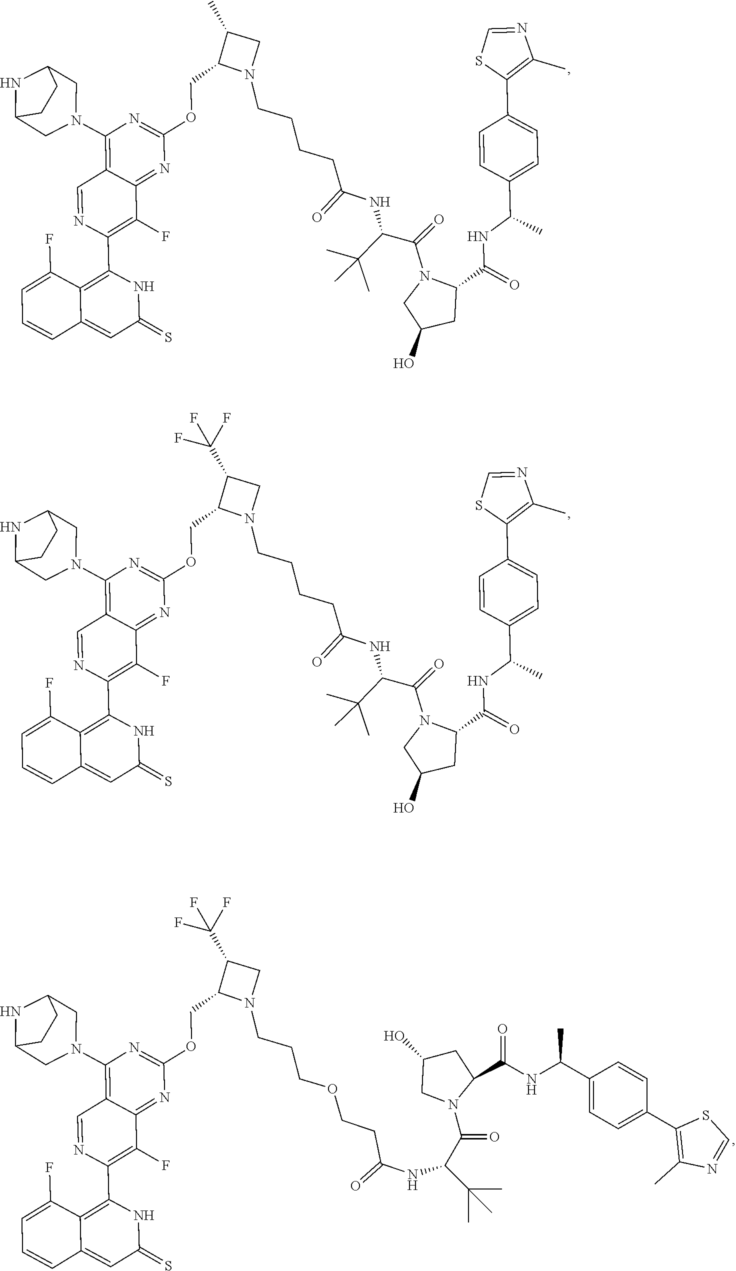

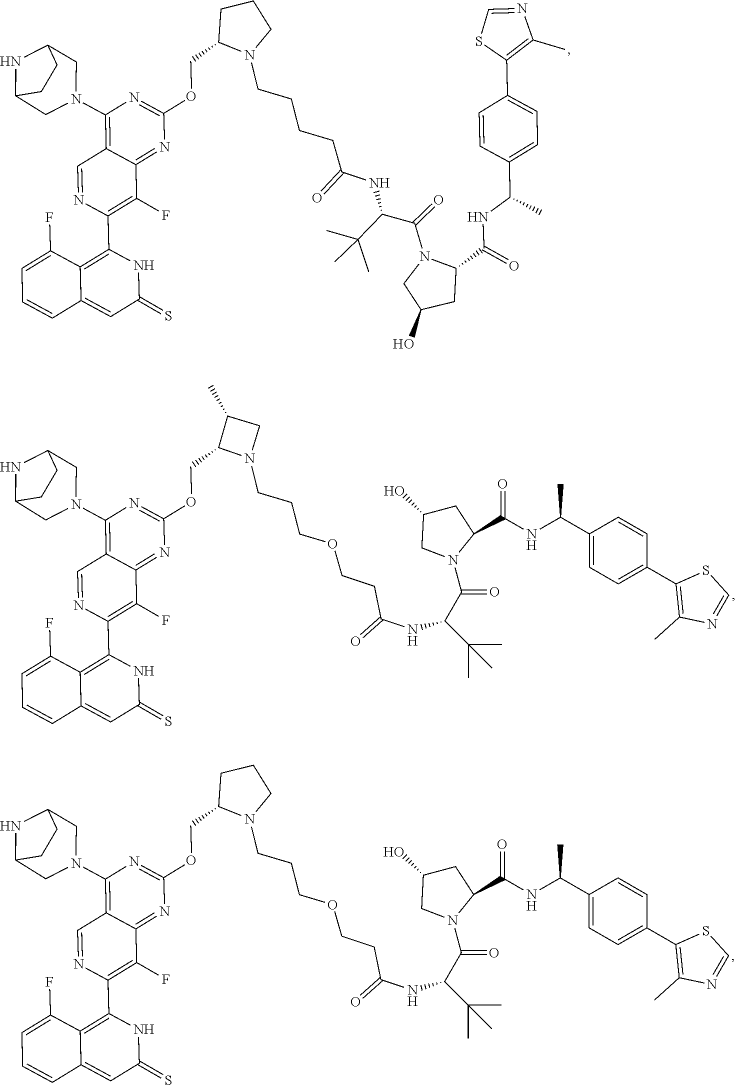 [SMILES: Cc1ncsc1-c1ccc([C@H](C)NC(=O)[C@@H]2C[C@@H](O)CN2C(=O)[C@@H](NC(=O)CCCCN2CCC[C@H]2COc2nc(N3CC4CCC(C3)N4)c3cnc(-c4[nH]c(=S)cc5cccc(F)c45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2C[C@@H](C)[C@H]2COc2nc(N3CC4CCC(C3)N4)c3cnc(-c4[nH]c(=S)cc5cccc(F)c45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cnc(-c4[nH]c(=S)cc5cccc(F)c45)c(F)c3n2)C(C)(C)C)cc1]

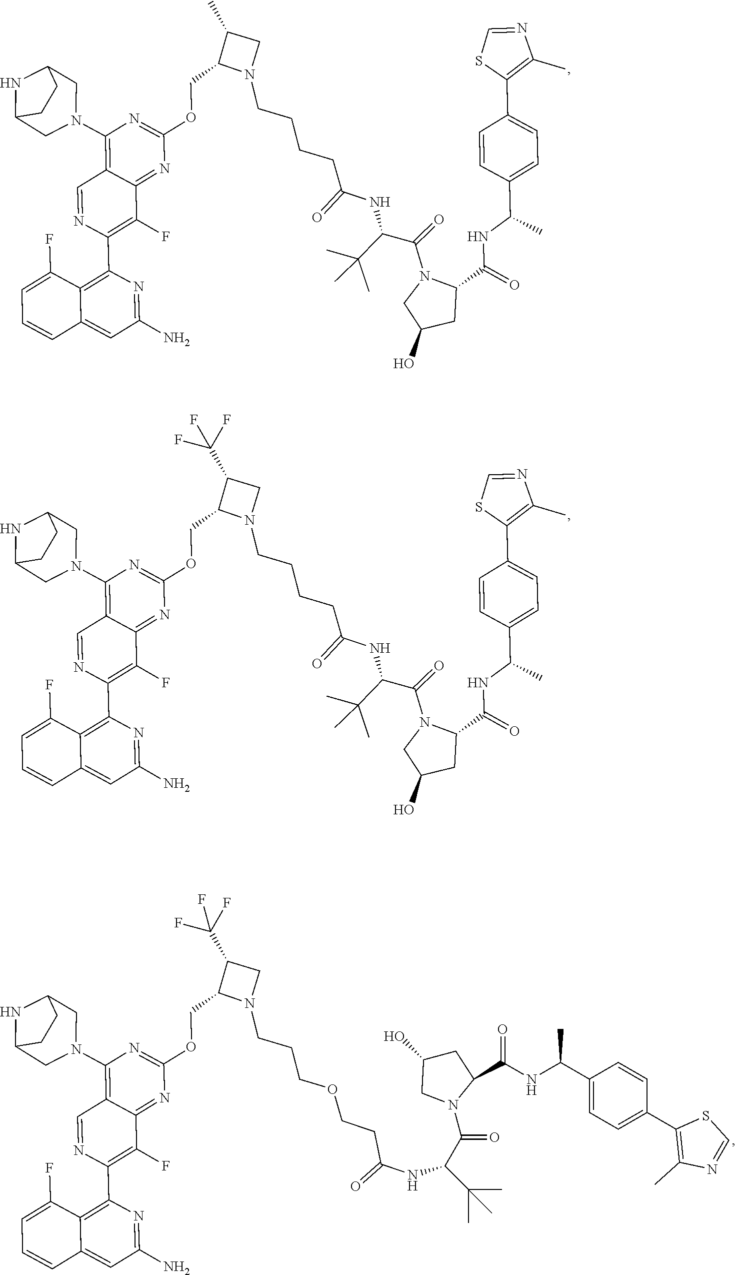

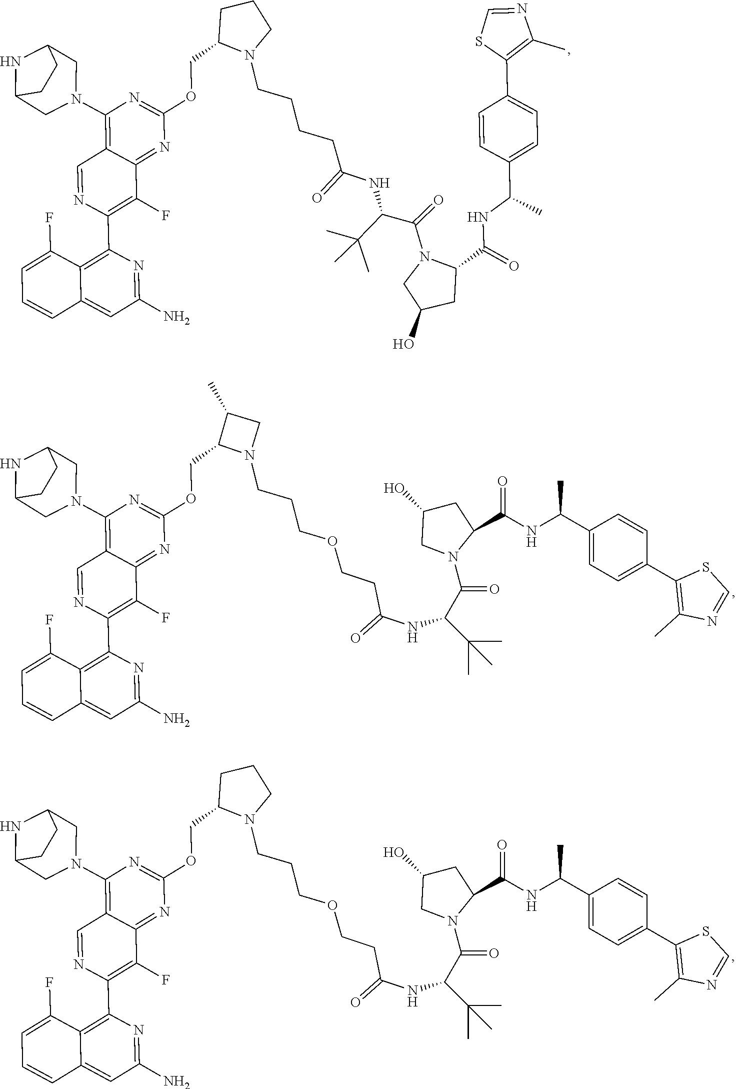 [SMILES: Cc1ncsc1-c1ccc([C@H](C)NC(=O)[C@@H]2C[C@@H](O)CN2C(=O)[C@@H](NC(=O)CCCCN2CCC[C@H]2COc2nc(N3CC4CCC(C3)N4)c3cnc(-c4nc(N)cc5cccc(F)c45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2C[C@@H](C)[C@H]2COc2nc(N3CC4CCC(C3)N4)c3cnc(-c4nc(N)cc5cccc(F)c45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cnc(-c4nc(N)cc5cccc(F)c45)c(F)c3n2)C(C)(C)C)cc1]

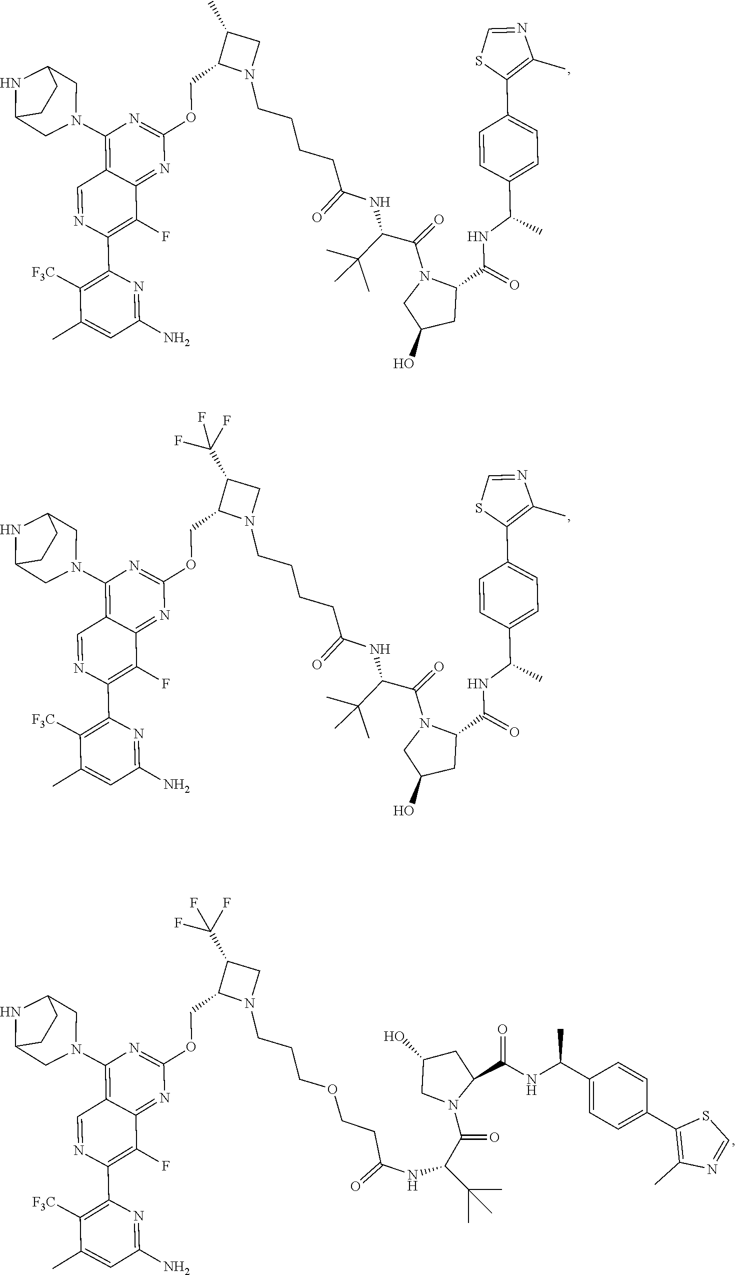 [SMILES: Cc1cc(N)nc(-c2ncc3c(N4CC5CCC(C4)N5)nc(OC[C@@H]4[C@H](C)CN4CCCCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F ; Cc1cc(N)nc(-c2ncc3c(N4CC5CCC(C4)N5)nc(OC[C@@H]4[C@H](C(F)(F)F)CN4CCCCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F ; Cc1cc(N)nc(-c2ncc3c(N4CC5CCC(C4)N5)nc(OC[C@@H]4[C@H](C(F)(F)F)CN4CCCOCCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F]

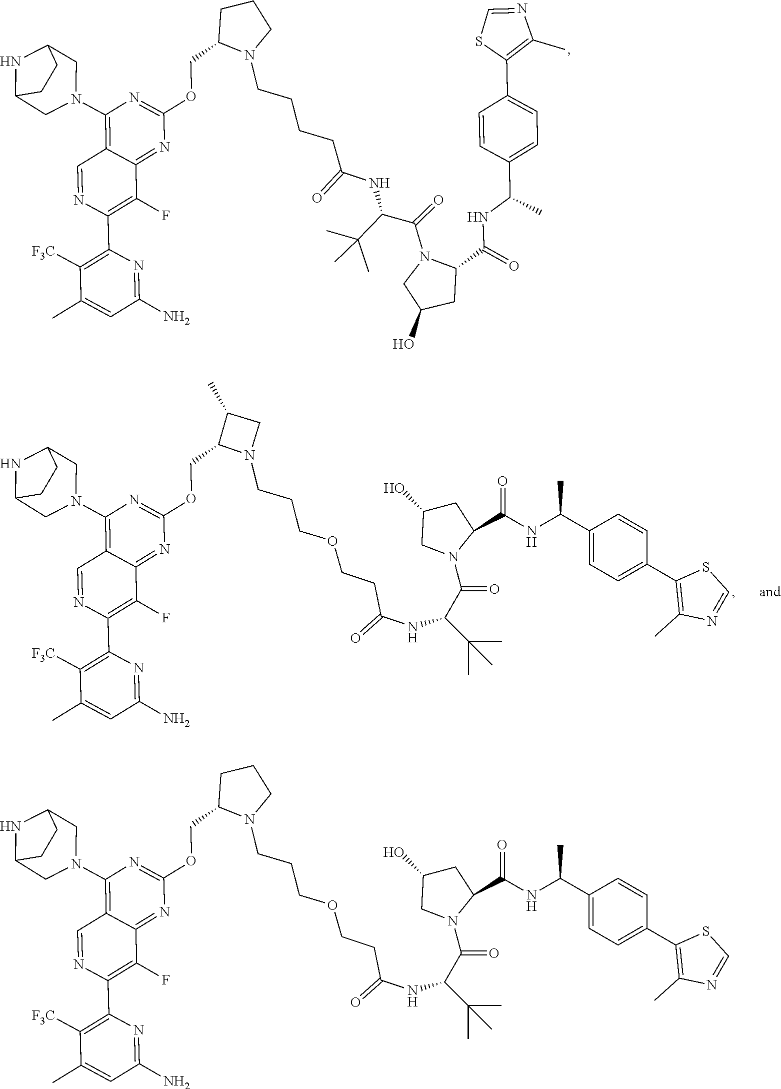 [SMILES: Cc1cc(N)nc(-c2ncc3c(N4CC5CCC(C4)N5)nc(OC[C@@H]4CCCN4CCCCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F ; Cc1cc(N)nc(-c2ncc3c(N4CC5CCC(C4)N5)nc(OC[C@@H]4[C@H](C)CN4CCCOCCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F ; Cc1cc(N)nc(-c2ncc3c(N4CC5CCC(C4)N5)nc(OC[C@@H]4CCCN4CCCOCCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F]

In some embodiments, the conjugate is selected from:

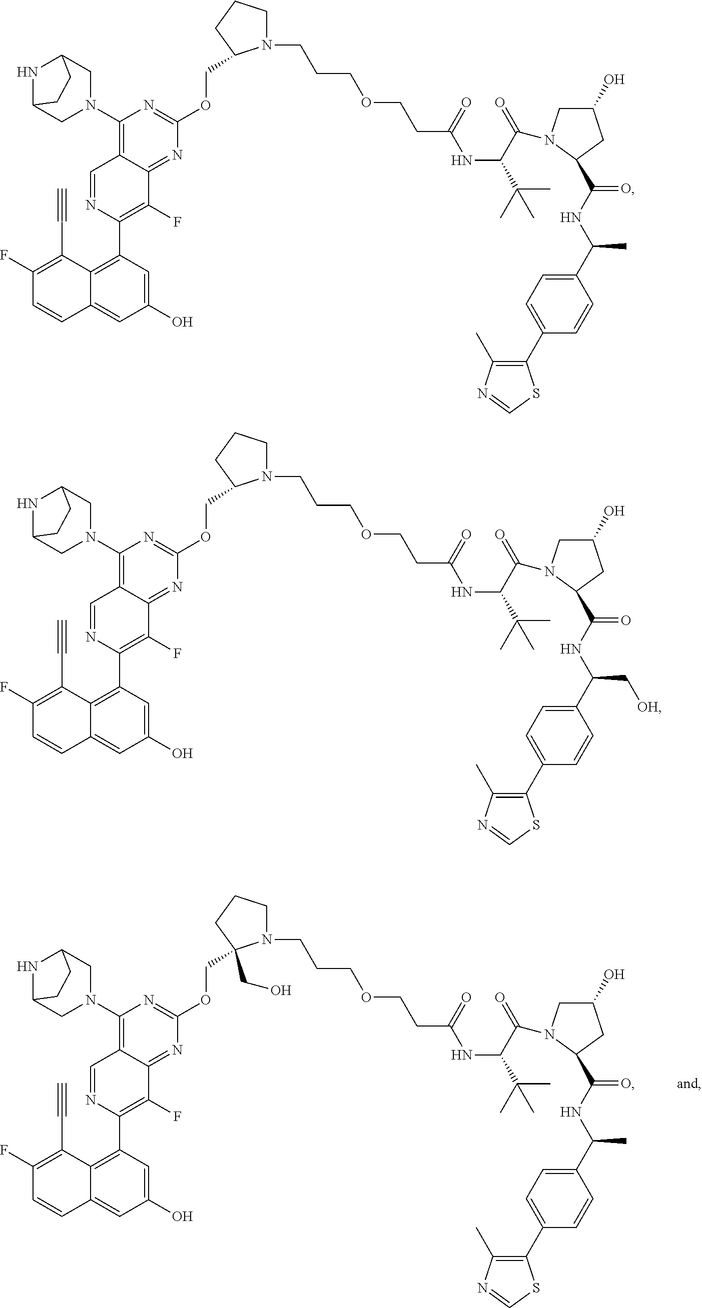 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6scnc6C)cc5)C(C)(C)C)nc4c3F)c12 ; C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](CO)c5ccc(-c6scnc6C)cc5)C(C)(C)C)nc4c3F)c12 ; C#Cc1c(F)ccc2cc(O)cc(-c3ncc4c(N5CC6CCC(C5)N6)nc(OC[C@]5(CO)CCCN5CCCOCCC(=O)N[C@H](C(=O)N5C[C@H](O)C[C@H]5C(=O)N[C@@H](C)c5ccc(-c6scnc6C)cc5)C(C)(C)C)nc4c3F)c12 ; "and,"]

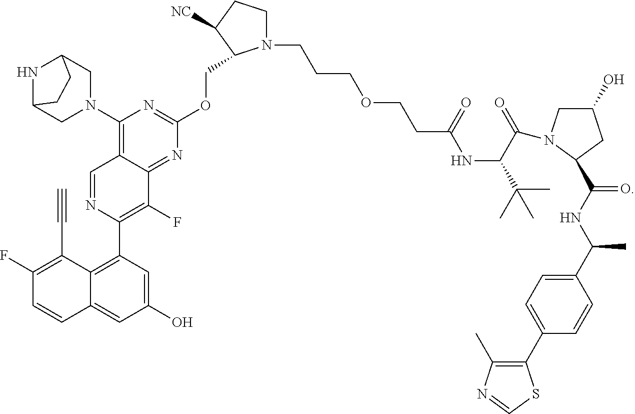 [SMILES: C#Cc1c(F)ccc2cc(O)cc(-c3ncc4c(N5CC6CCC(C5)N6)nc(OC[C@@H]5[C@@H](C#N)CCN5CCCOCCC(=O)N[C@H](C(=O)N5C[C@H](O)C[C@H]5C(=O)N[C@@H](C)c5ccc(-c6scnc6C)cc5)C(C)(C)C)nc4c3F)c12]

In some embodiments, the conjugate is selected from:

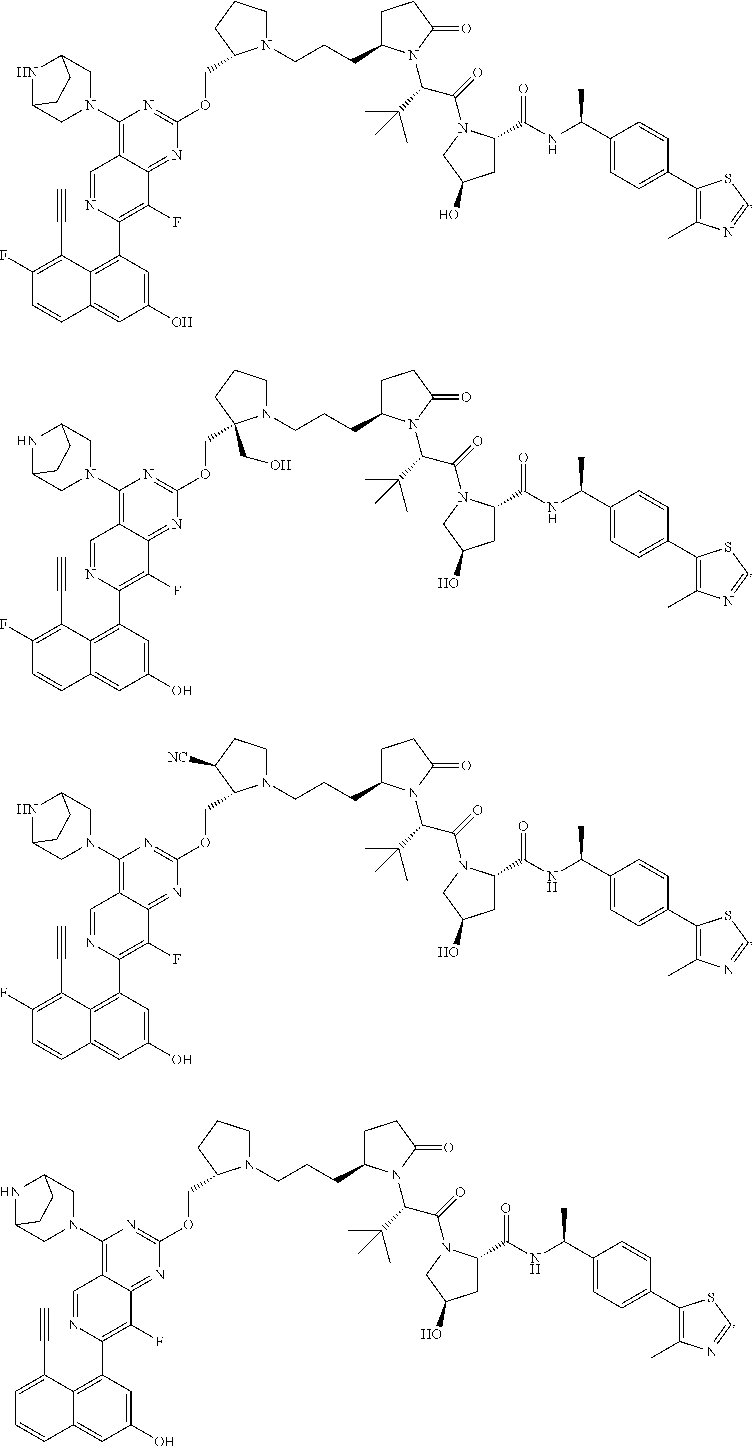

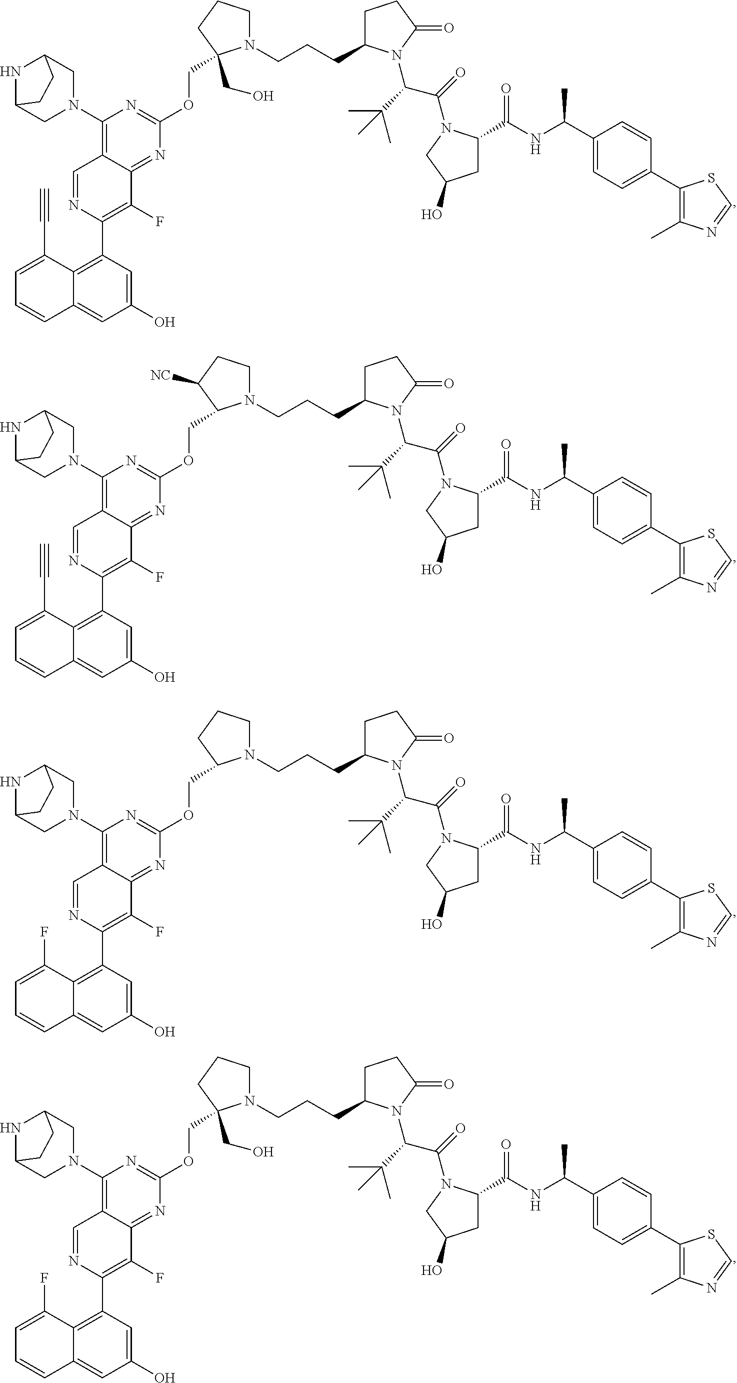

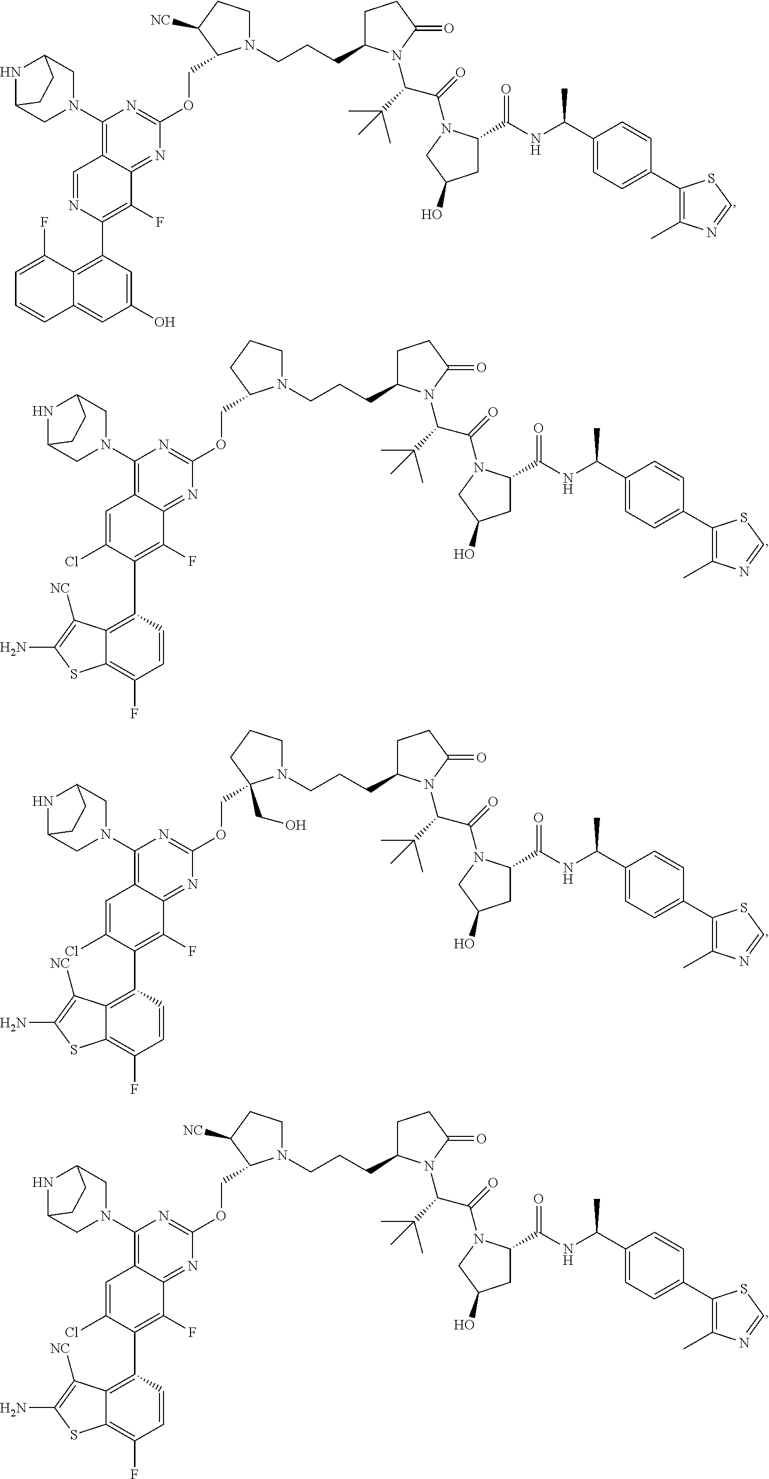

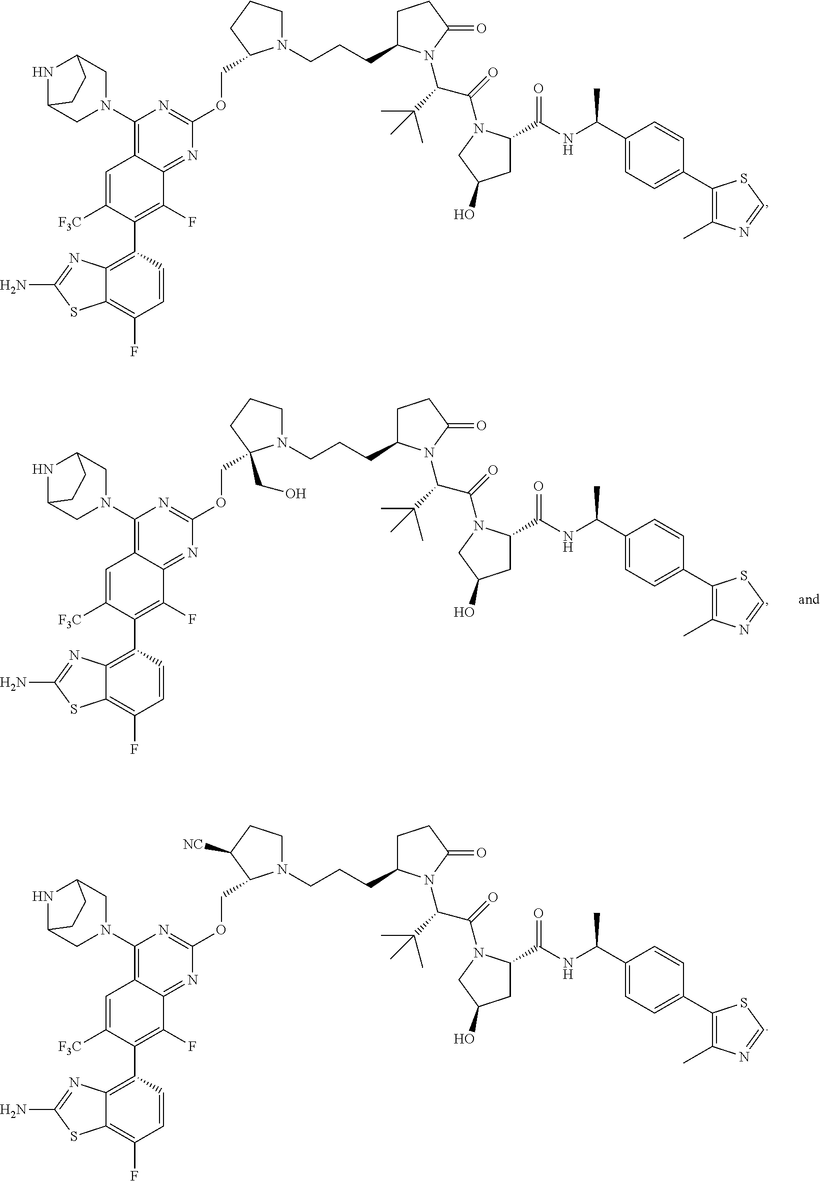

In some embodiments, the conjugate is selected from:

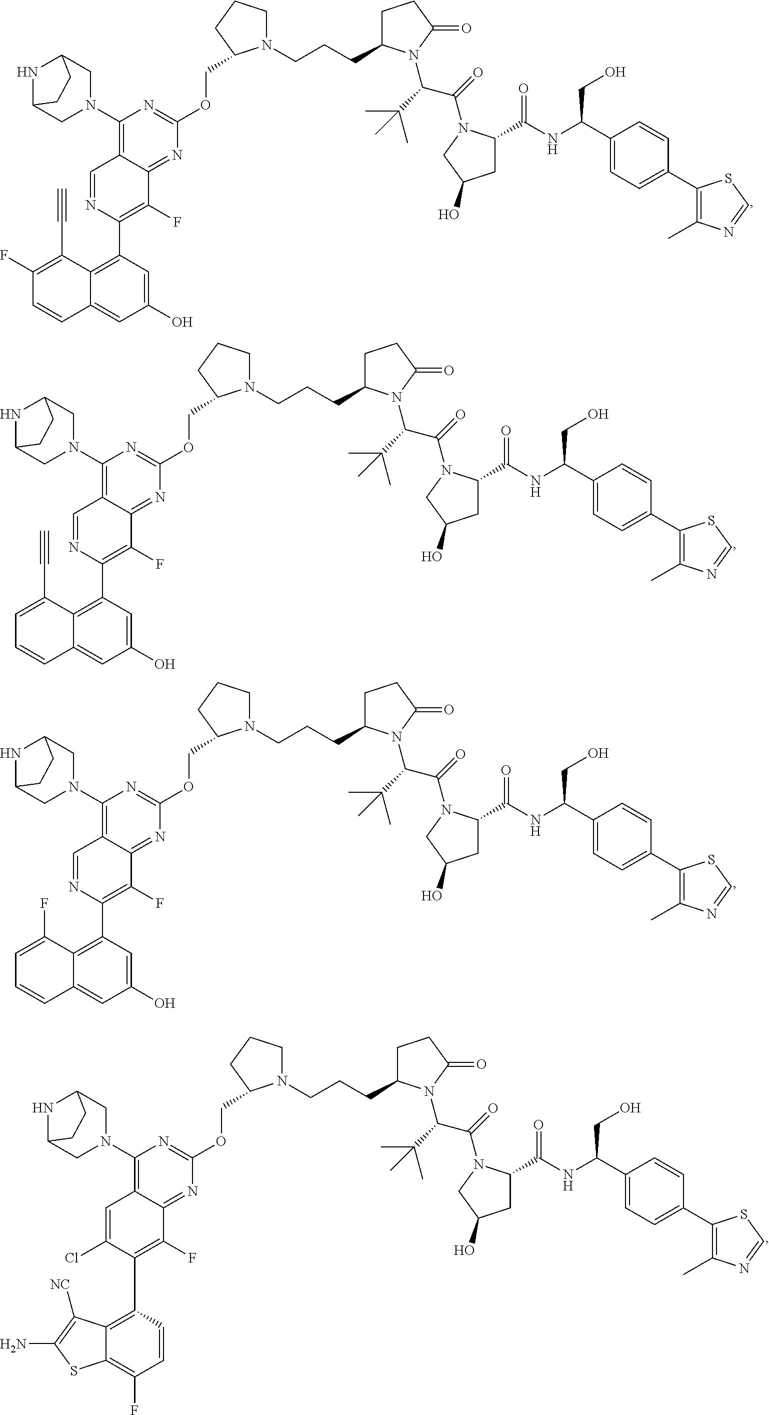

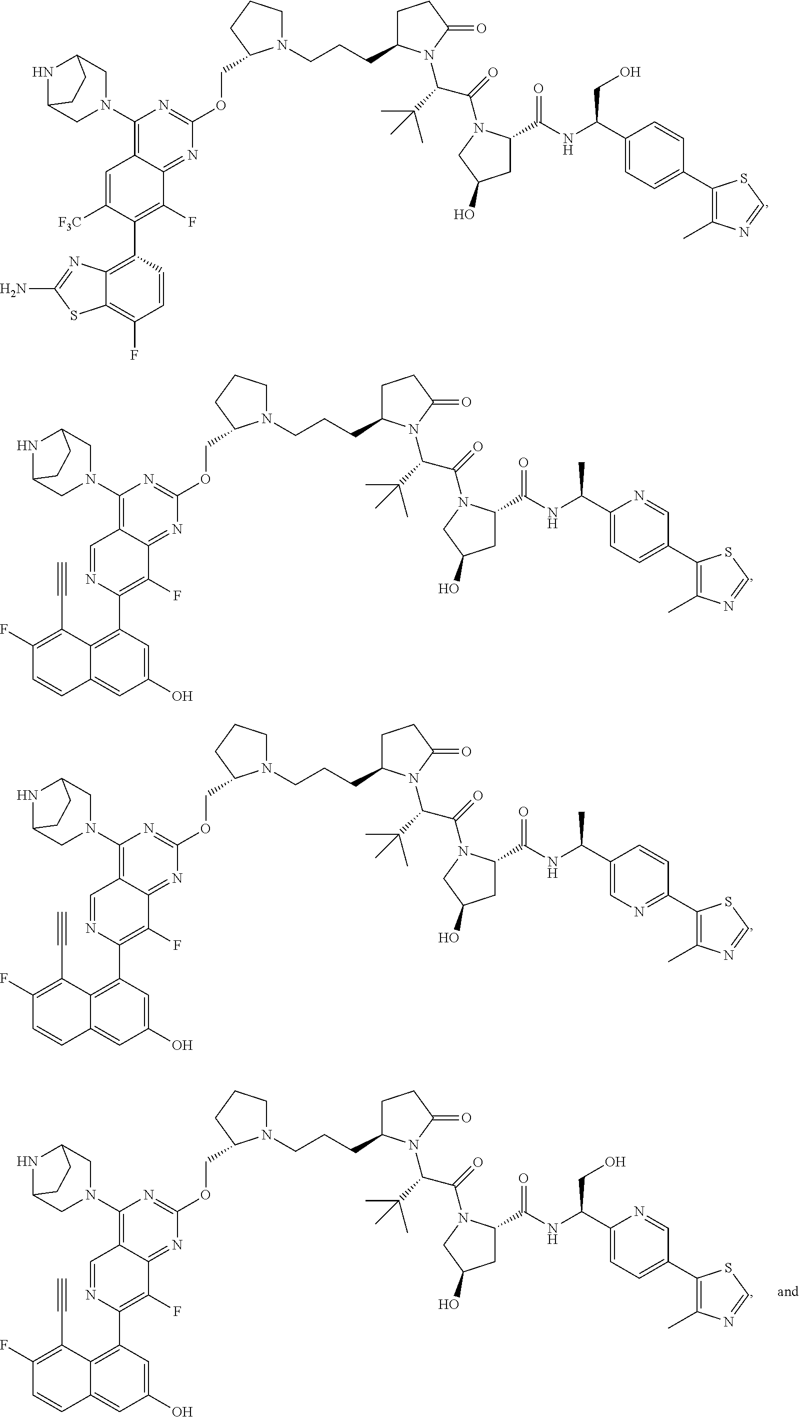

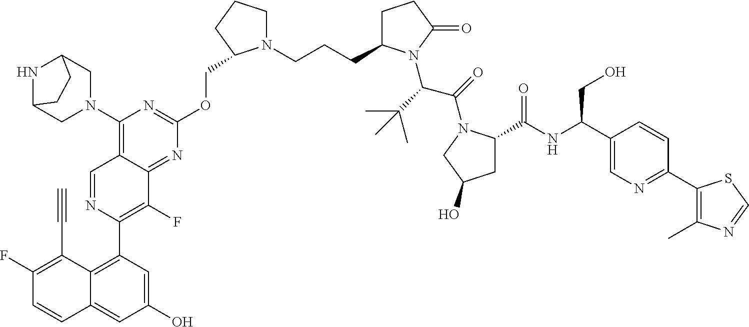 [SMILES: C#Cc1c(F)ccc2cc(O)cc(-c3ncc4c(N5CC6CCC(C5)N6)nc(OC[C@@H]5CCCN5CCC[C@H]5CCC(=O)N5[C@H](C(=O)N5C[C@H](O)C[C@H]5C(=O)N[C@@H](CO)c5ccc(-c6scnc6C)nc5)C(C)(C)C)nc4c3F)c12]

In embodiments, the conjugate is selected from:

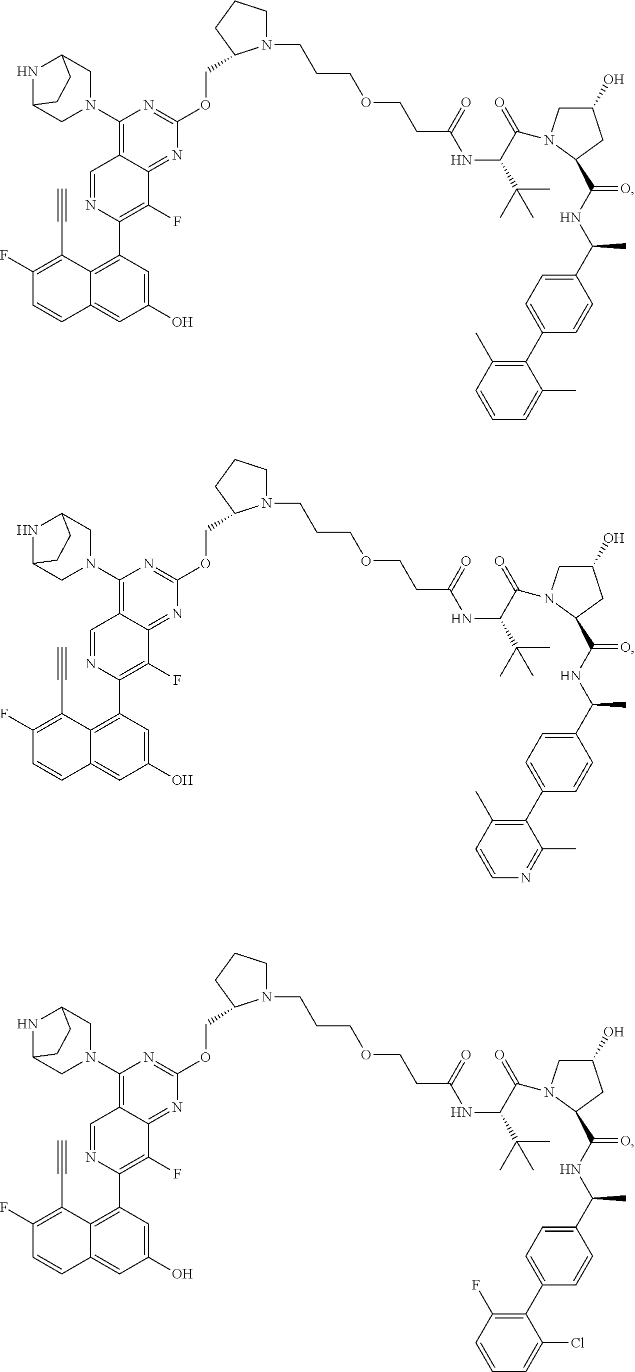 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(C)cccc6C)cc5)C(C)(C)C)nc4c3F)c12 ; C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(C)ccnc6C)cc5)C(C)(C)C)nc4c3F)c12 ; C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(F)cccc6Cl)cc5)C(C)(C)C)nc4c3F)c12]

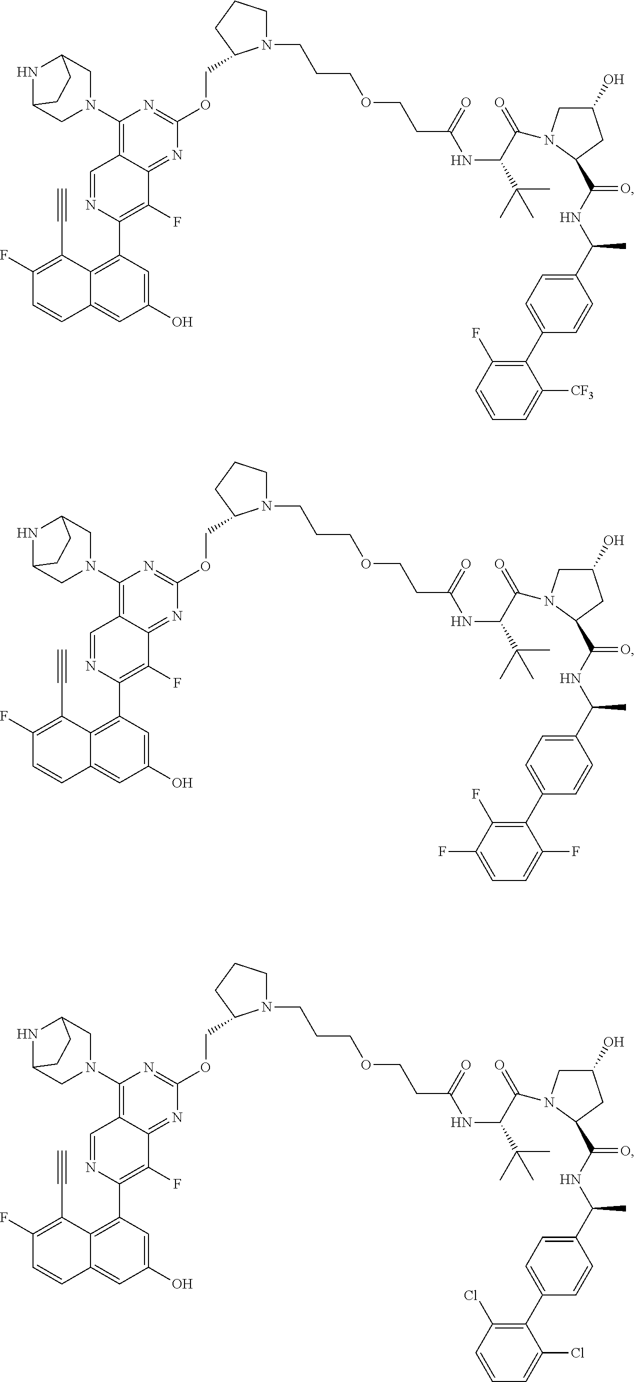

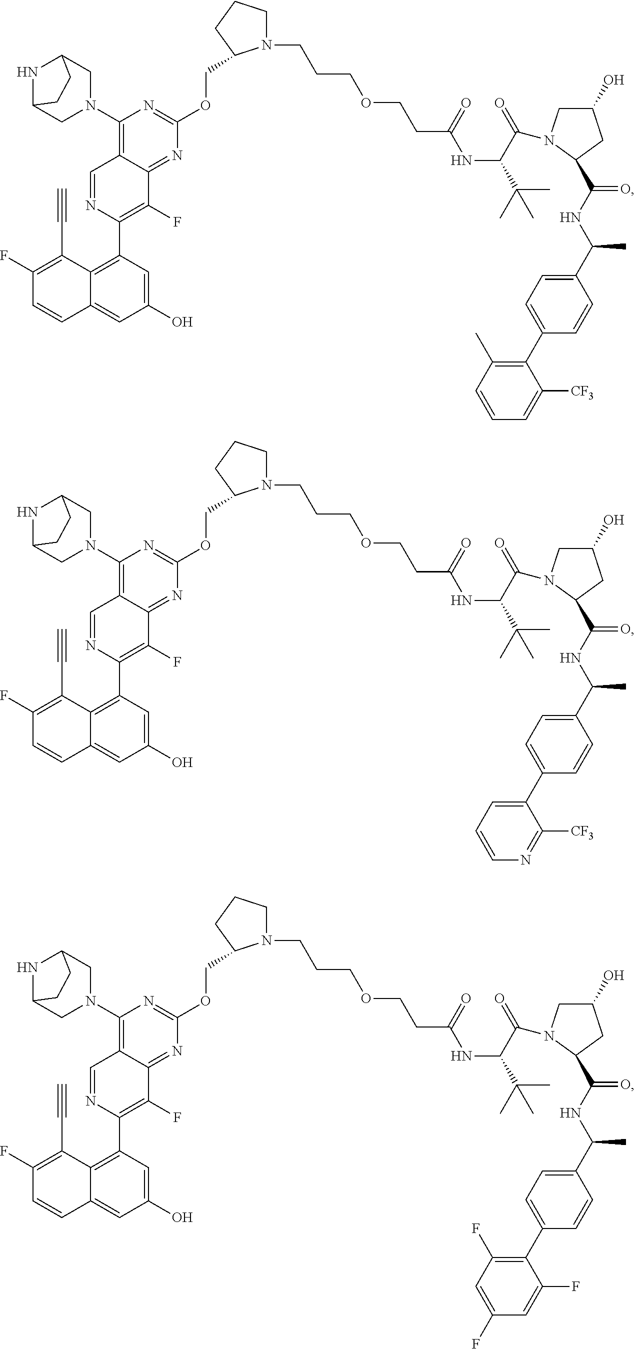

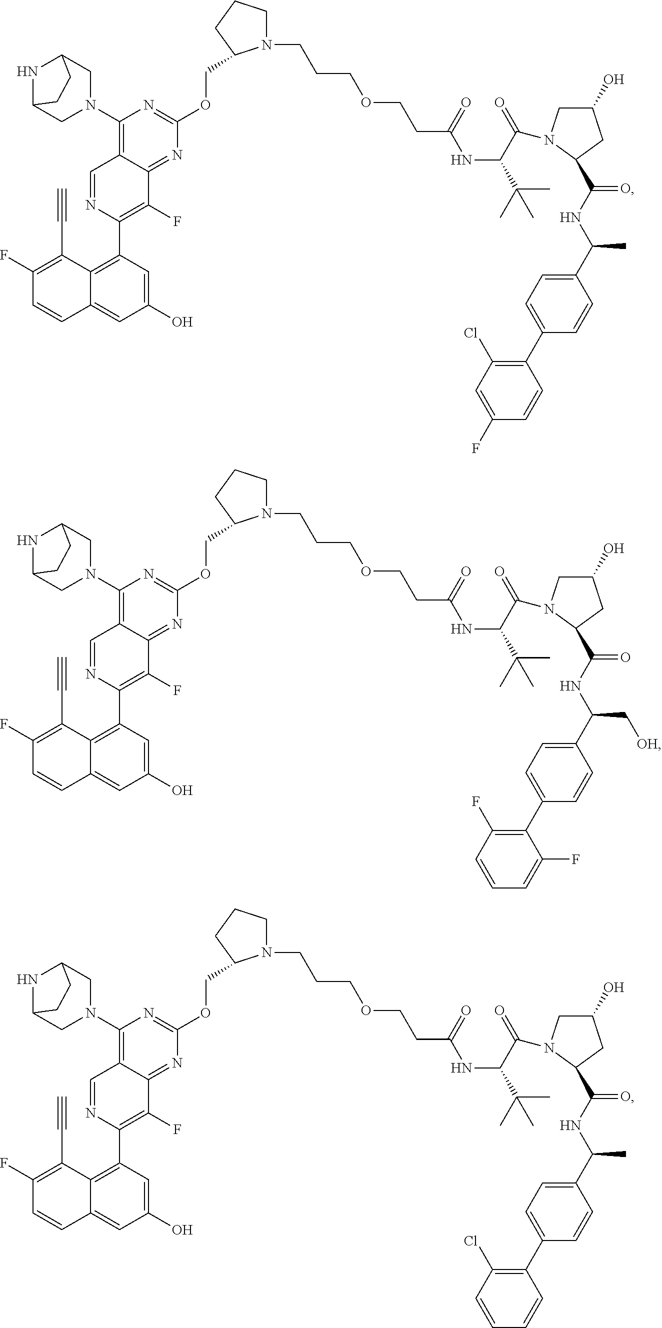 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6ccc(F)cc6Cl)cc5)C(C)(C)C)nc4c3F)c12 ; C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](CO)c5ccc(-c6c(F)cccc6F)cc5)C(C)(C)C)nc4c3F)c12 ; C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6ccccc6Cl)cc5)C(C)(C)C)nc4c3F)c12]

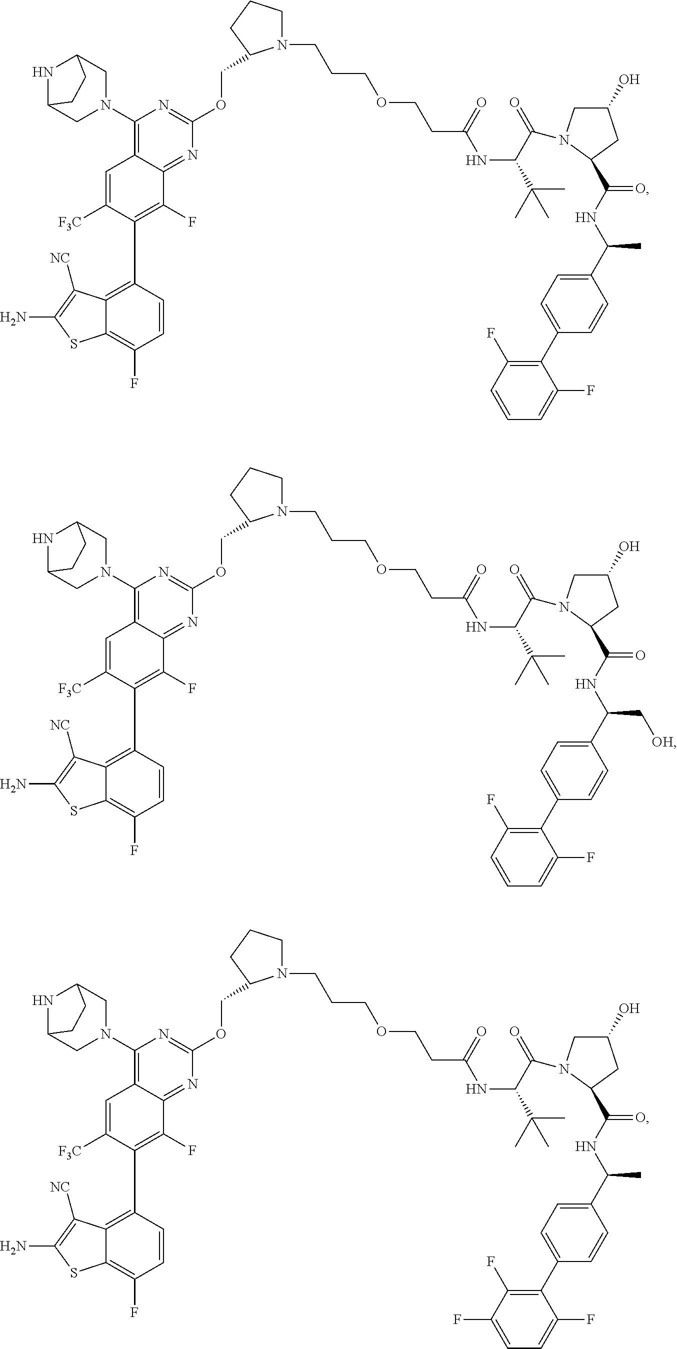

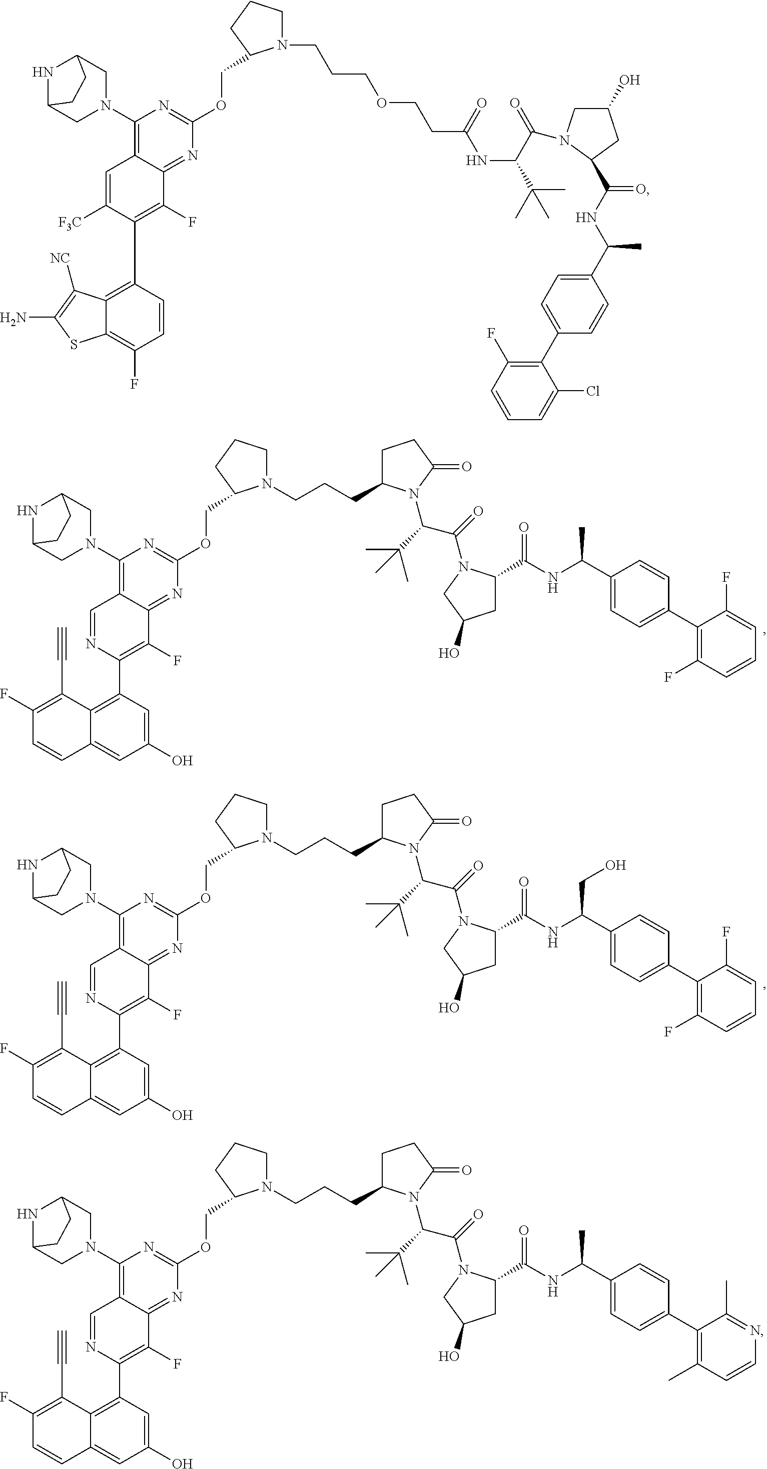

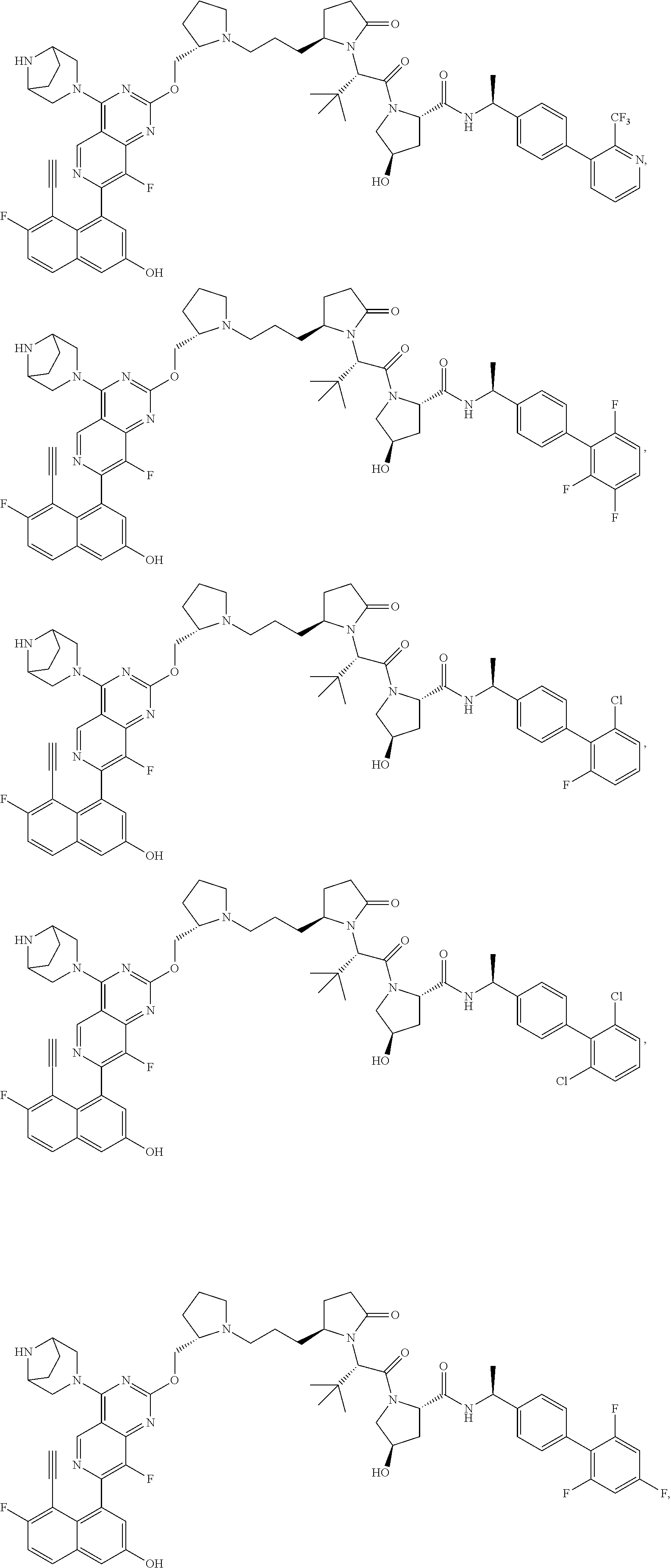

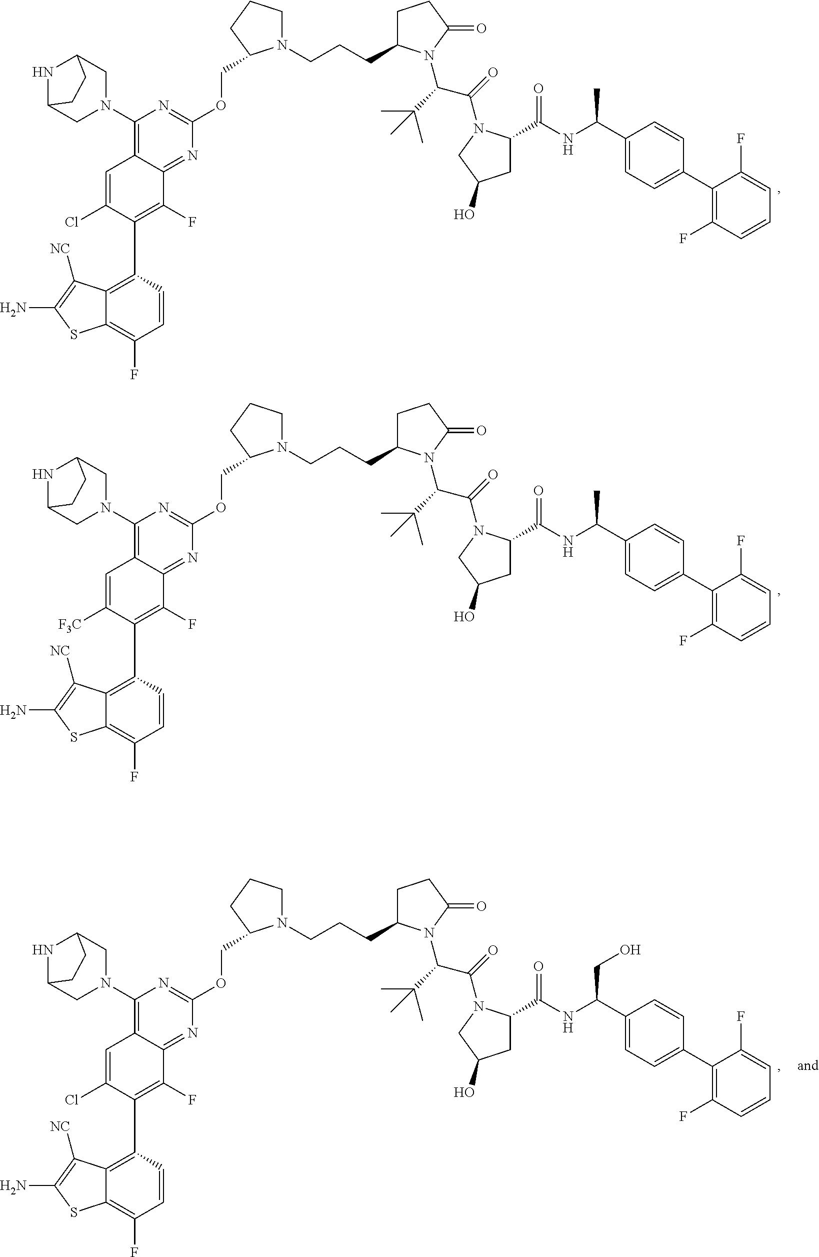

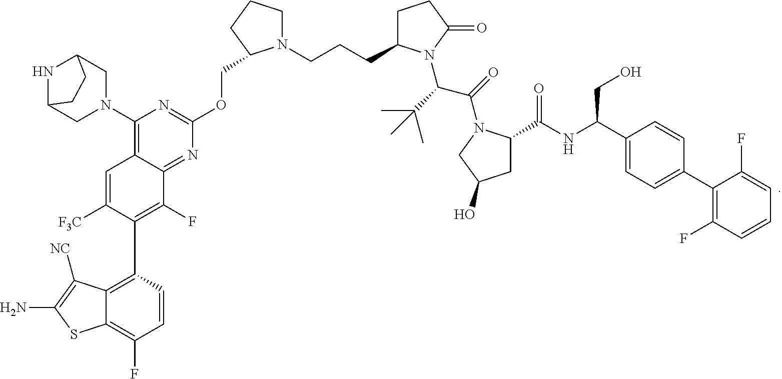

Still further conjugates are selected from:

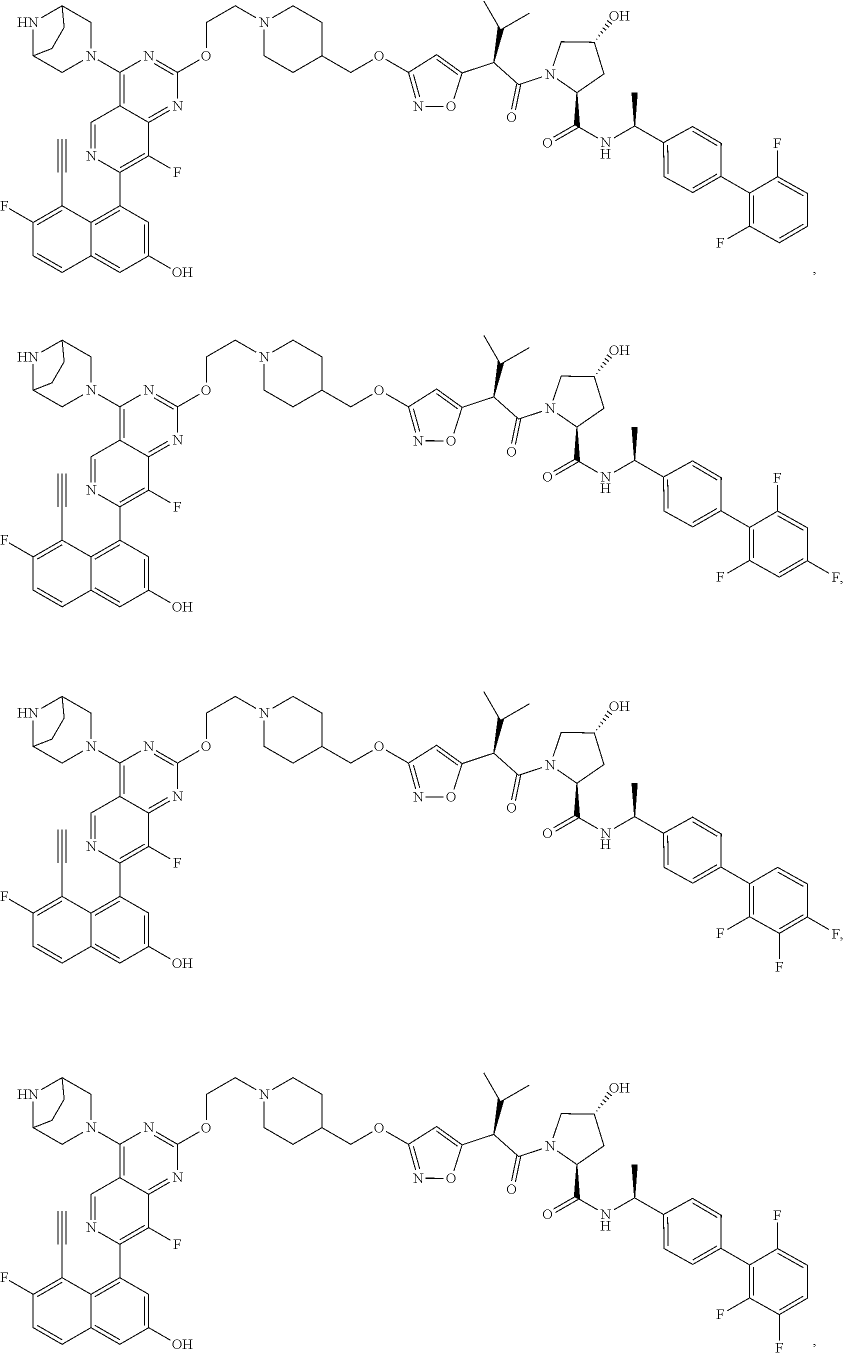

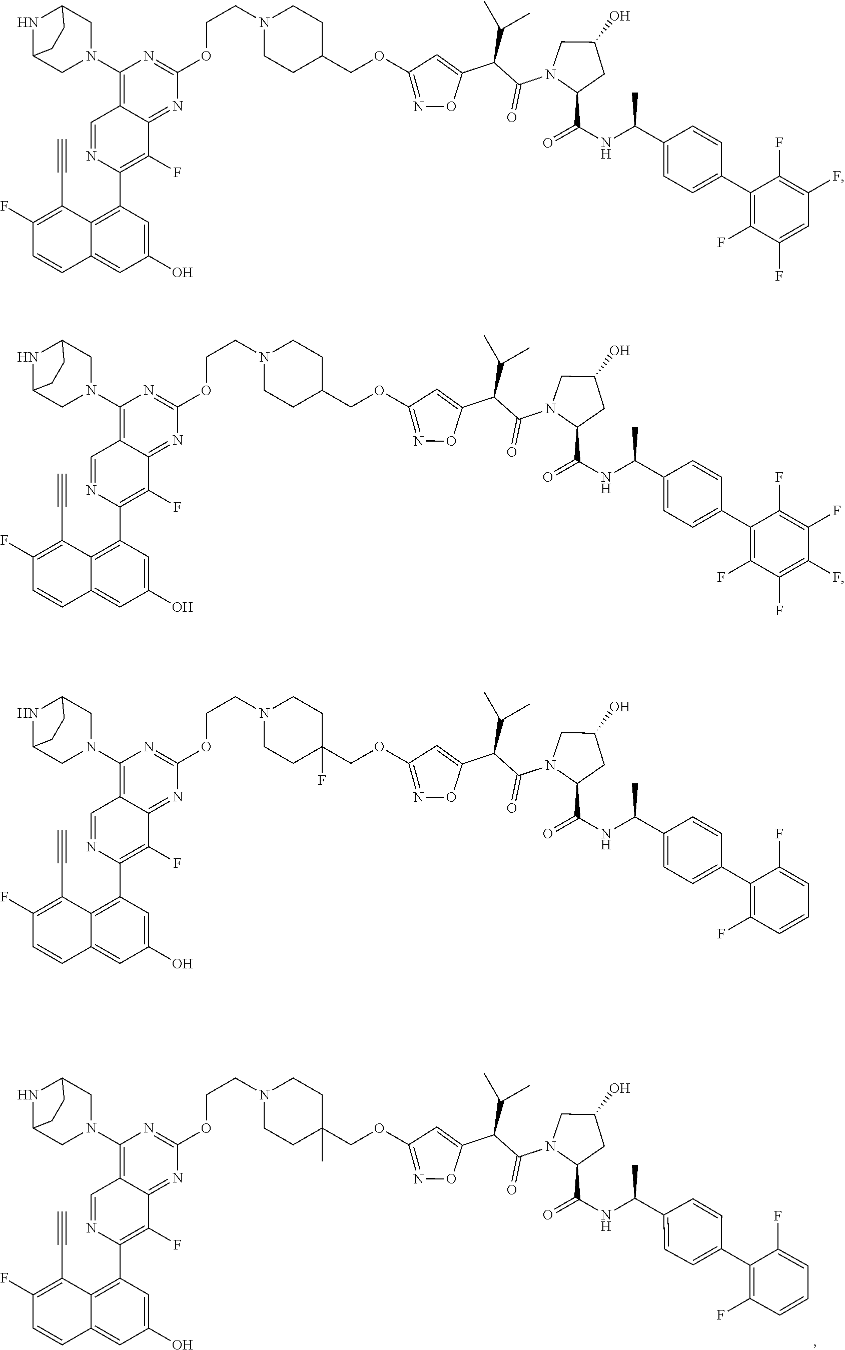

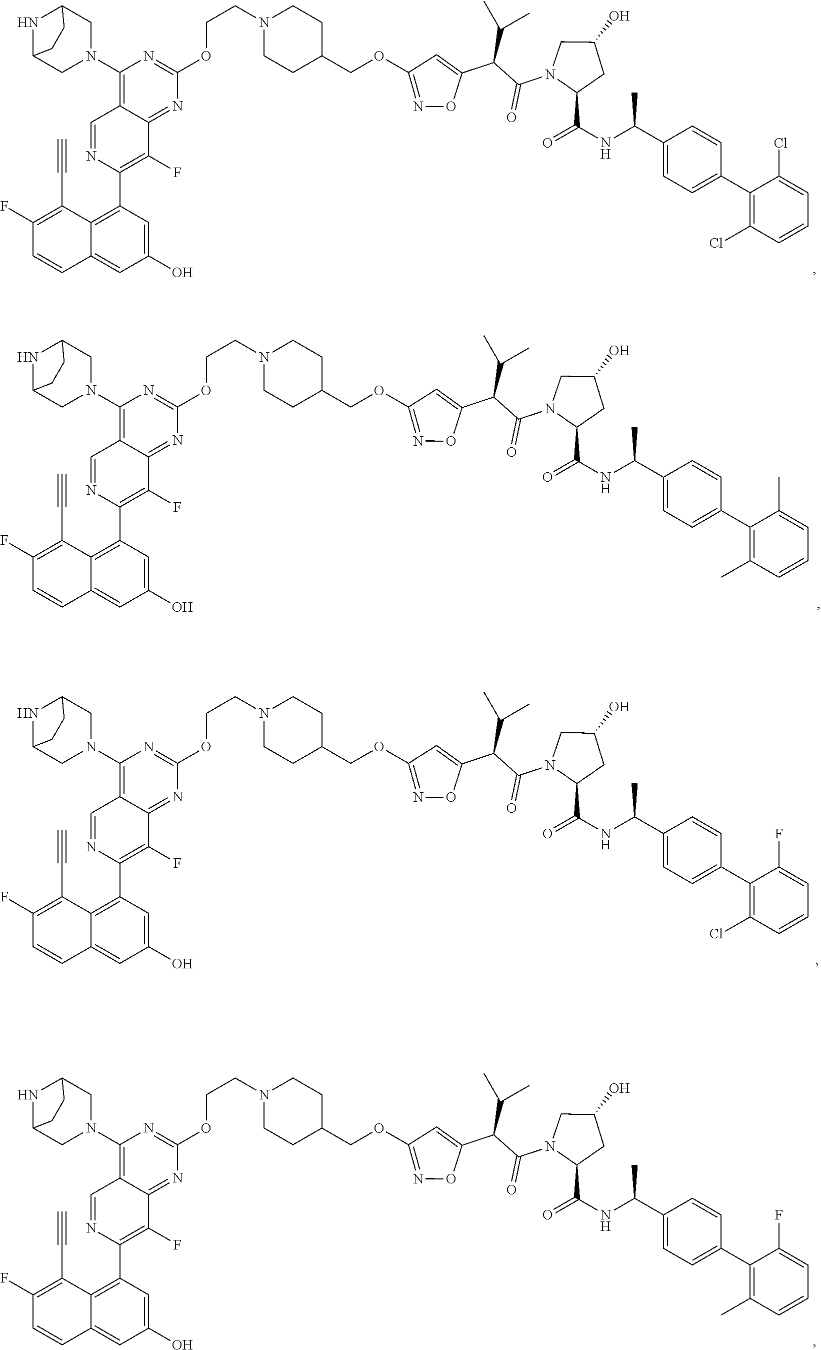

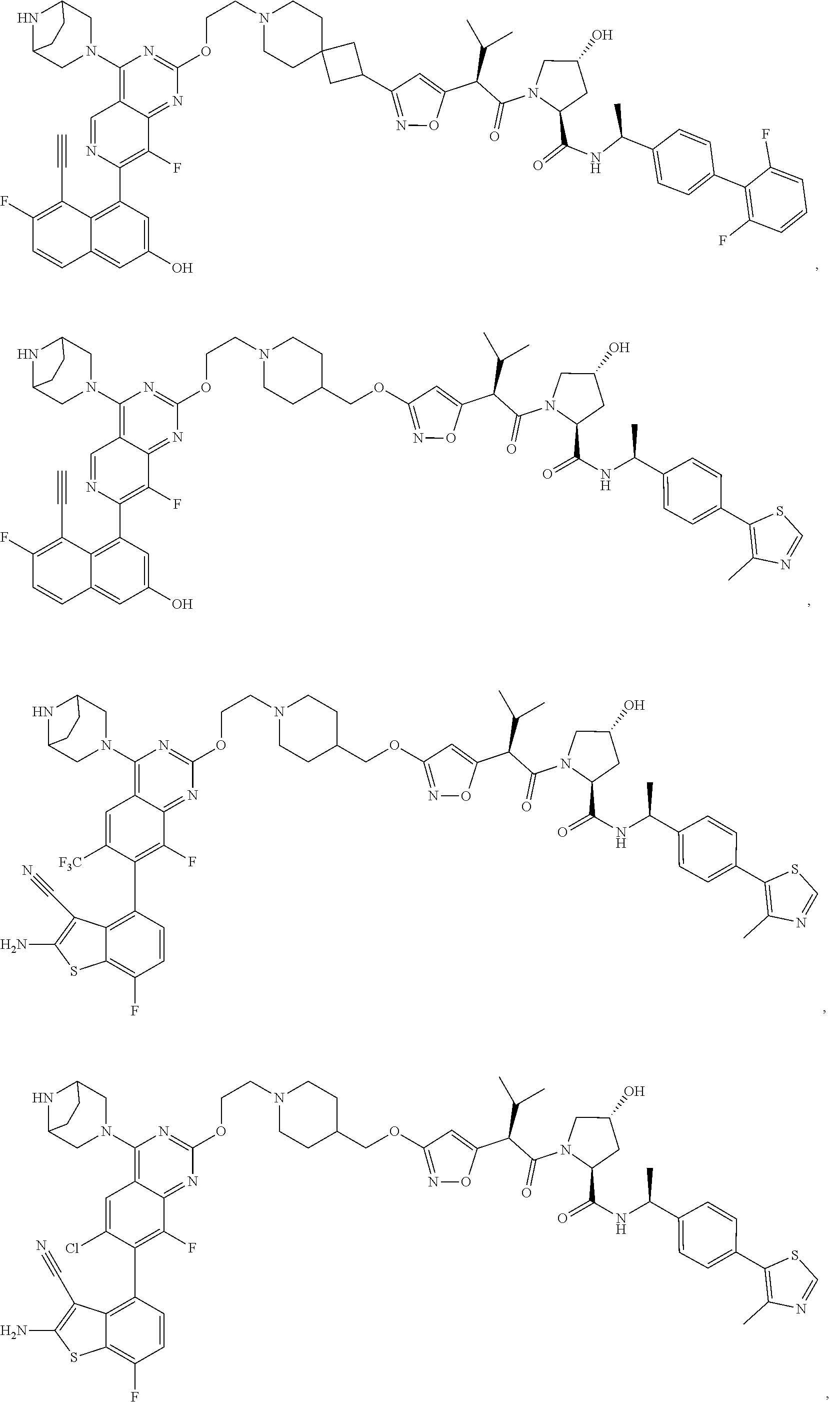

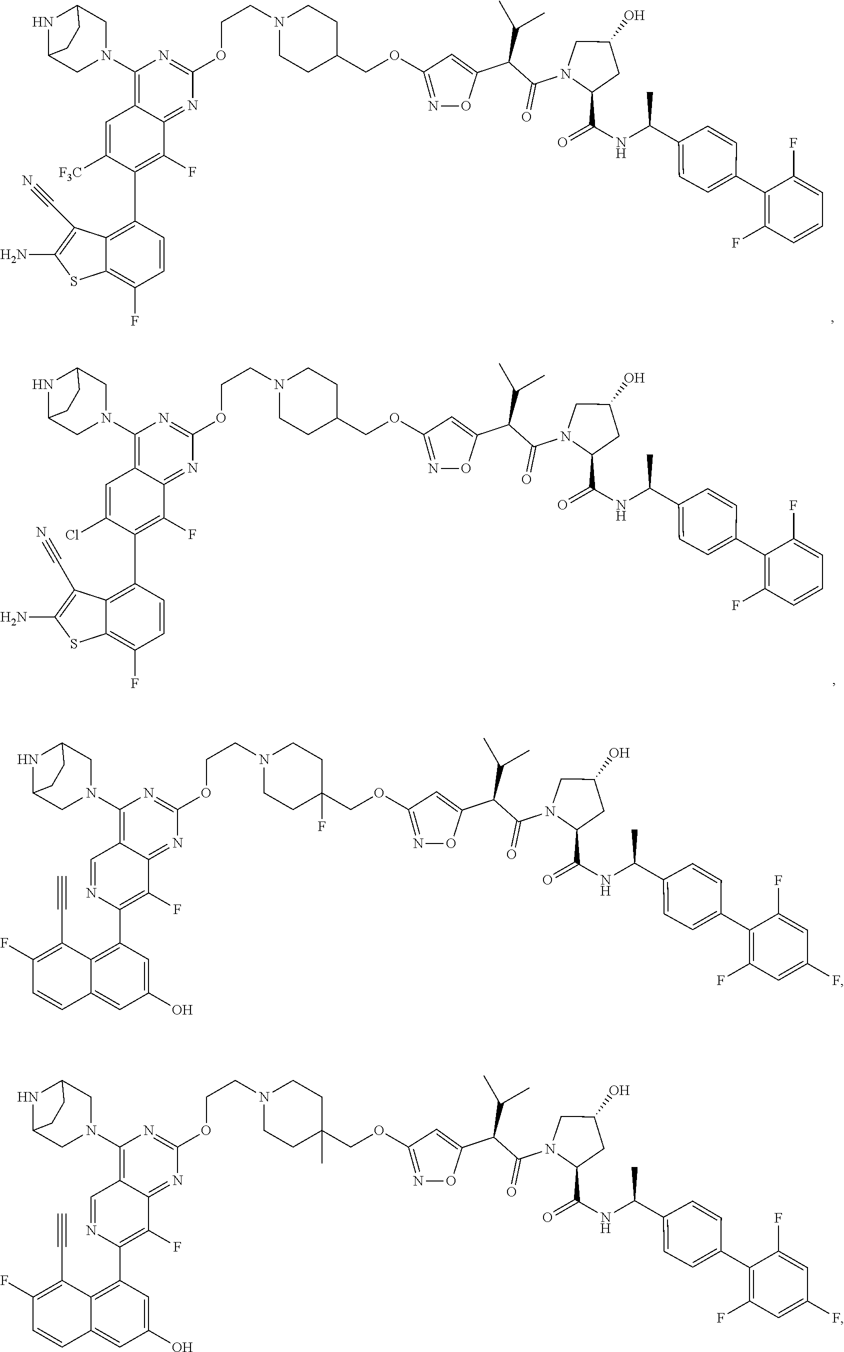

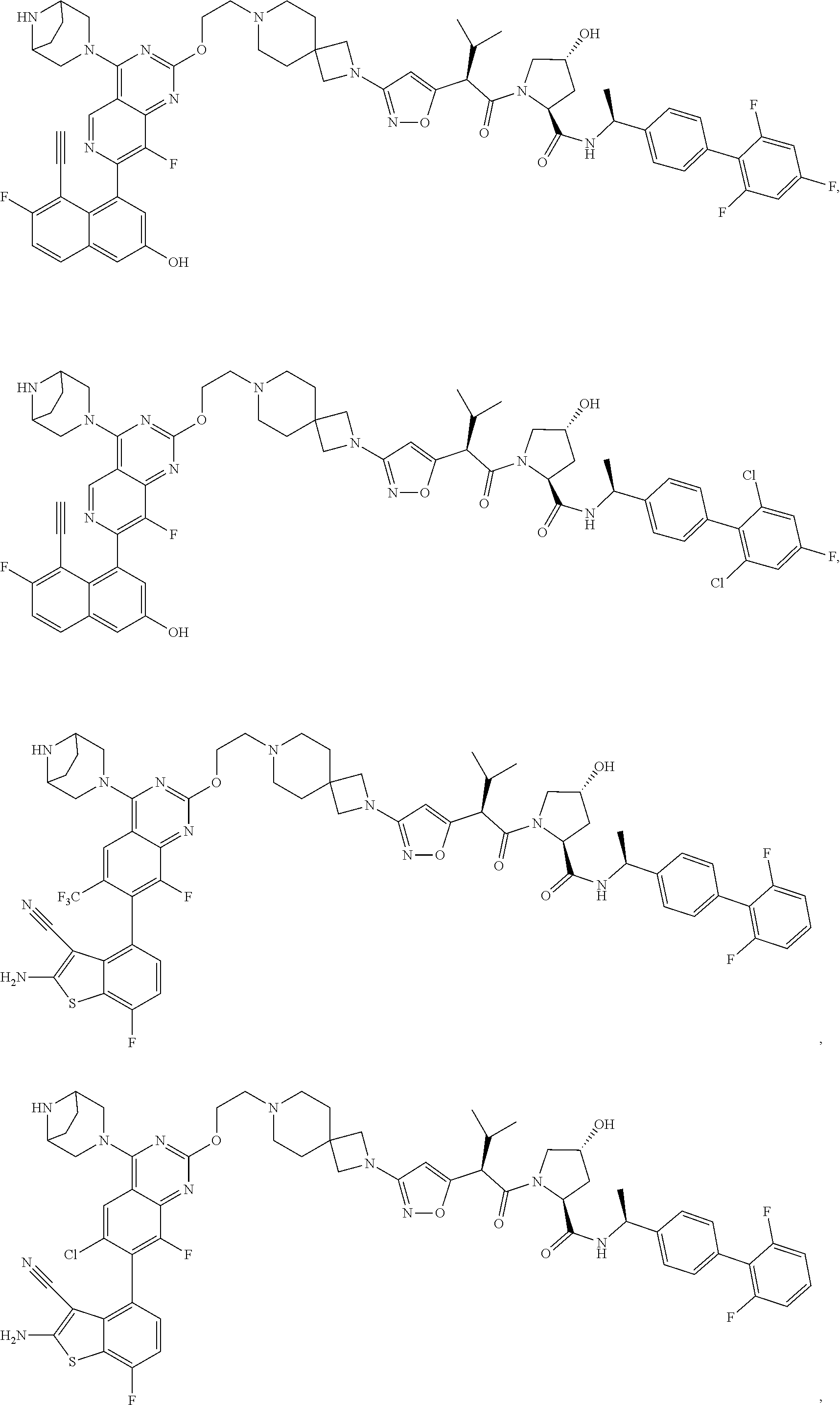

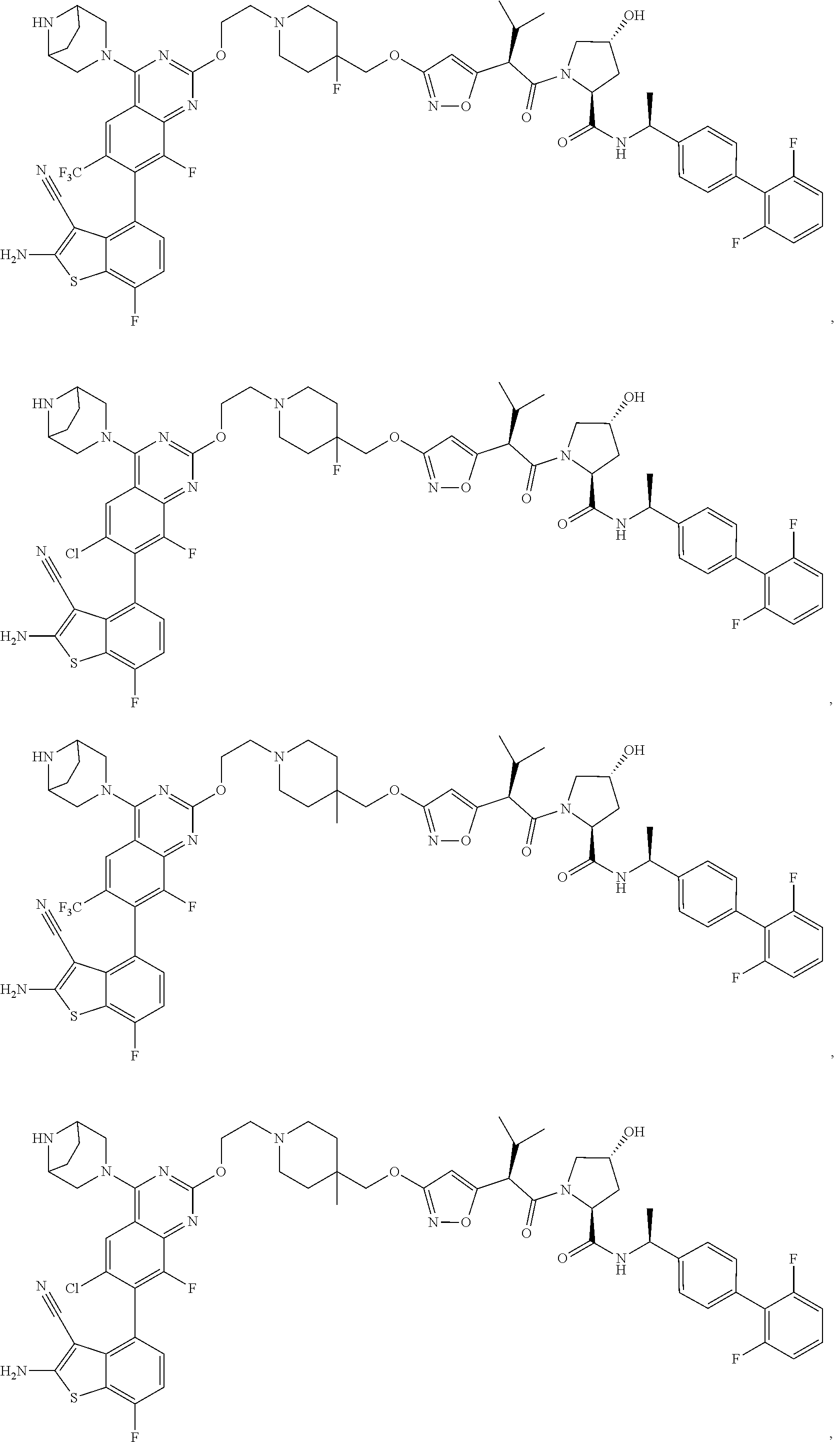

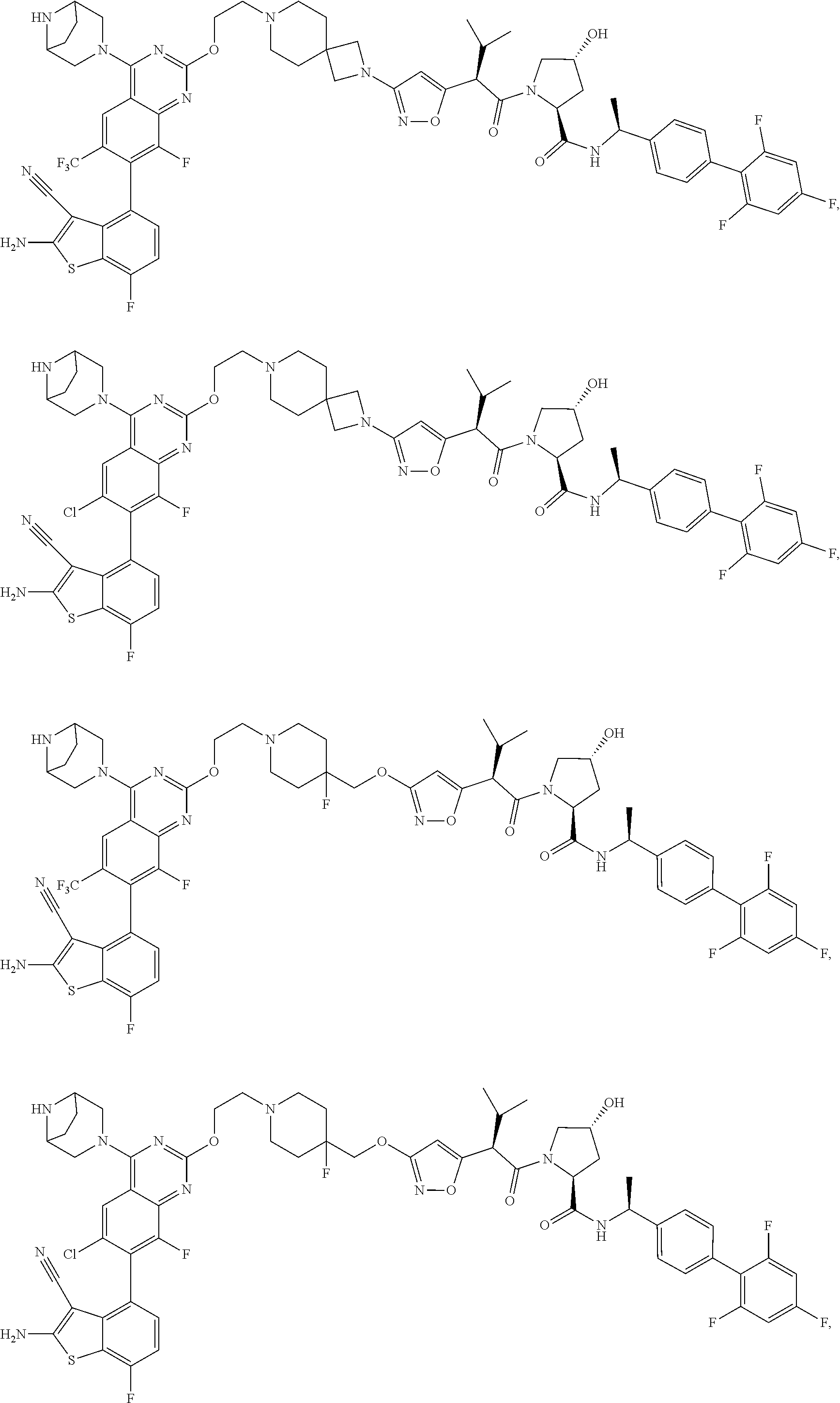

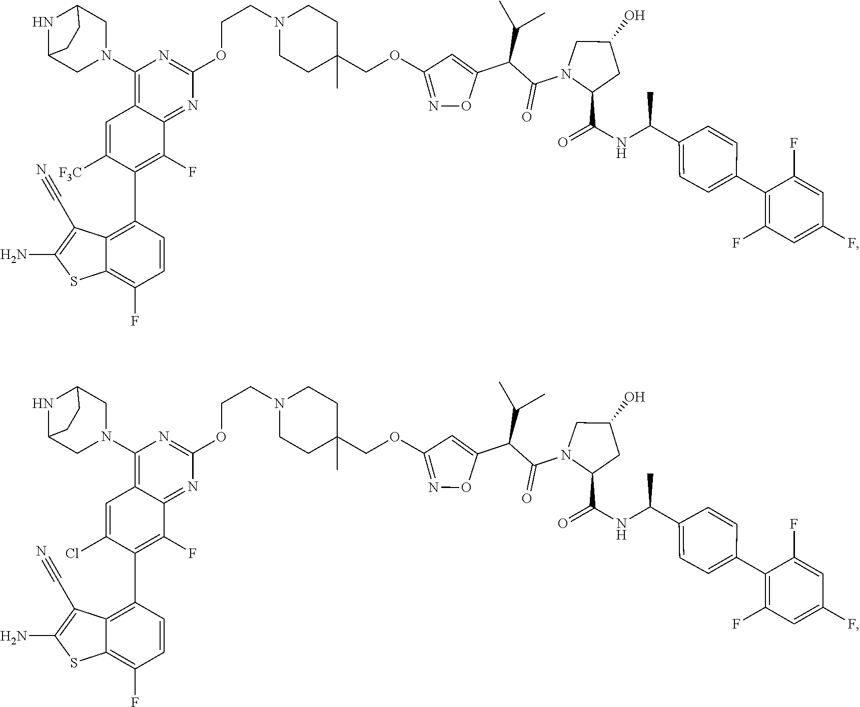 [SMILES: CC(C)[C@@H](C(=O)N1C[C@H](O)C[C@H]1C(=O)N[C@@H](C)c1ccc(-c2c(F)cc(F)cc2F)cc1)c1cc(OCC2(C)CCN(CCOc3nc(N4CC5CCC(C4)N5)c4cc(C(F)(F)F)c(-c5ccc(F)c6sc(N)c(C#N)c56)c(F)c4n3)CC2)no1 ; CC(C)[C@@H](C(=O)N1C[C@H](O)C[C@H]1C(=O)N[C@@H](C)c1ccc(-c2c(F)cc(F)cc2F)cc1)c1cc(OCC2(C)CCN(CCOc3nc(N4CC5CCC(C4)N5)c4cc(Cl)c(-c5ccc(F)c6sc(N)c(C#N)c56)c(F)c4n3)CC2)no1]

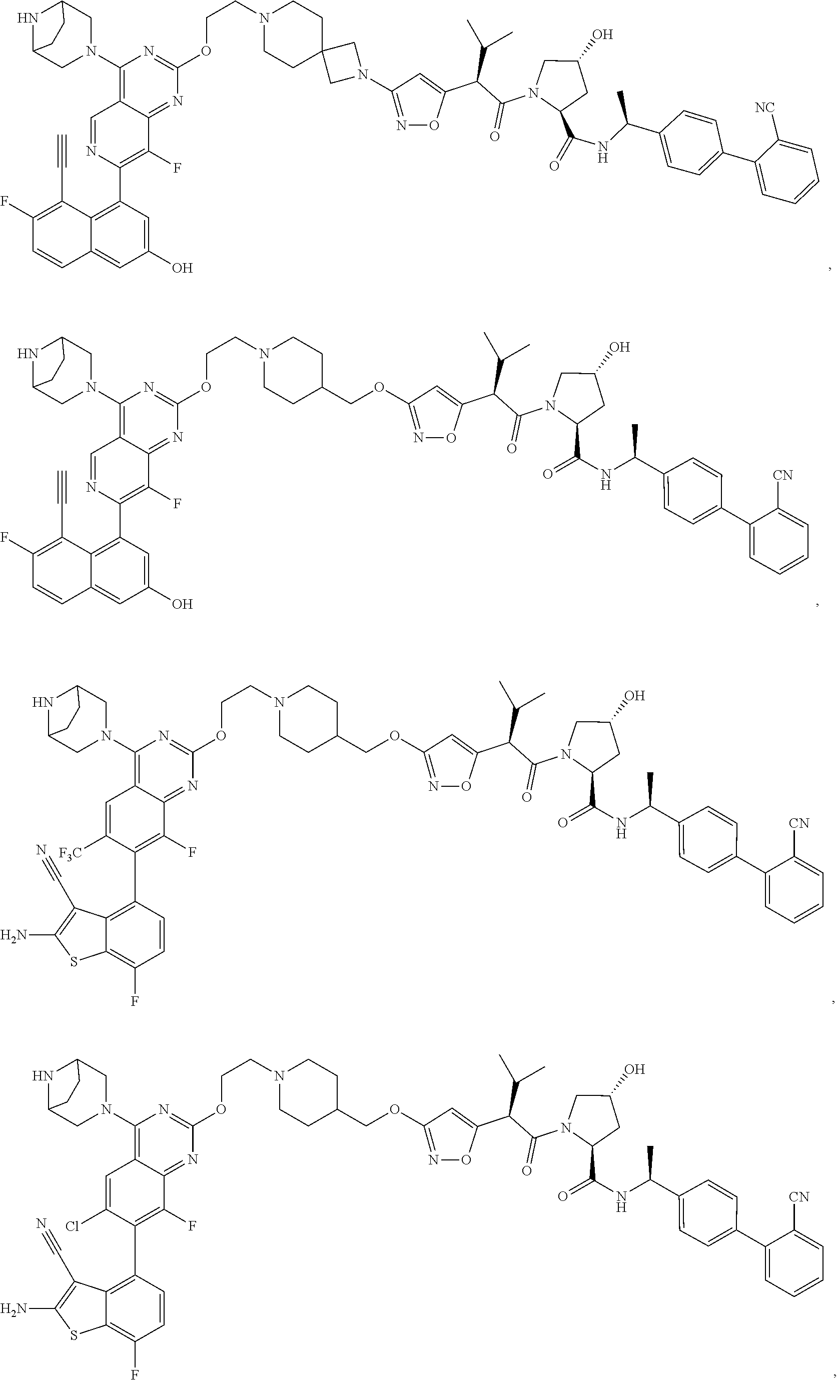

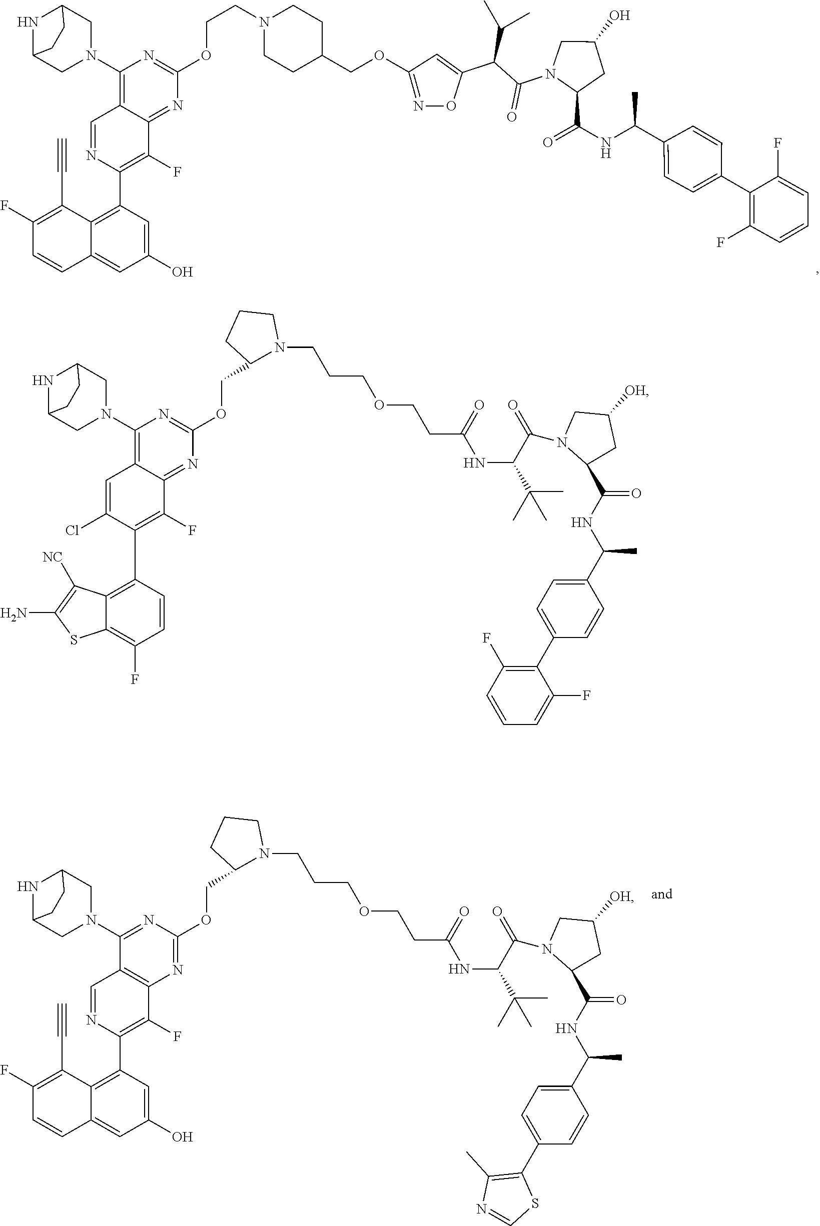

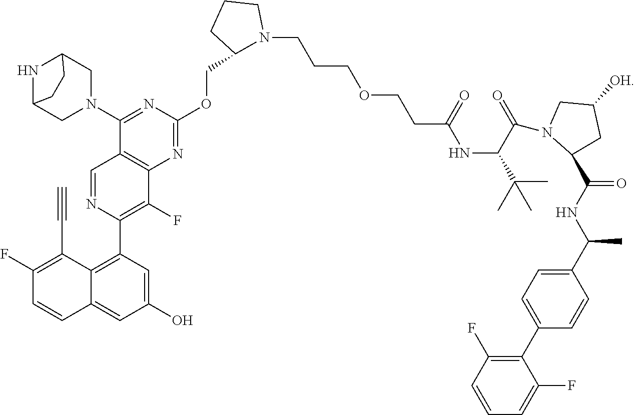 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(F)cccc6F)cc5)C(C)(C)C)nc4c3F)c12]

In embodiments, there are provided conjugates of Formula P, or pharmaceutically acceptable salt thereof:

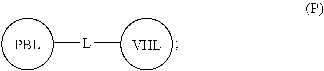

wherein PBL is a protein binding ligand capable of binding to and/or inhibiting a target protein; L is a bivalent linker that connects PBL to a Von Hippel-Lindau (VHL) moiety;
wherein VHL binds to a ubiquitin ligase; and
wherein the VHL ligand is:

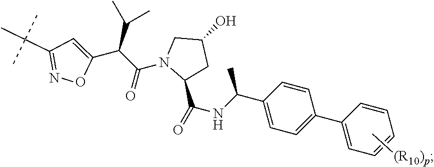

and
wherein p is an integer from 0 to 5; and each R¹⁰ is independently selected from alkyl, cyano, and halogen.

In embodiments, the VHL ligand is selected from the group consisting of:

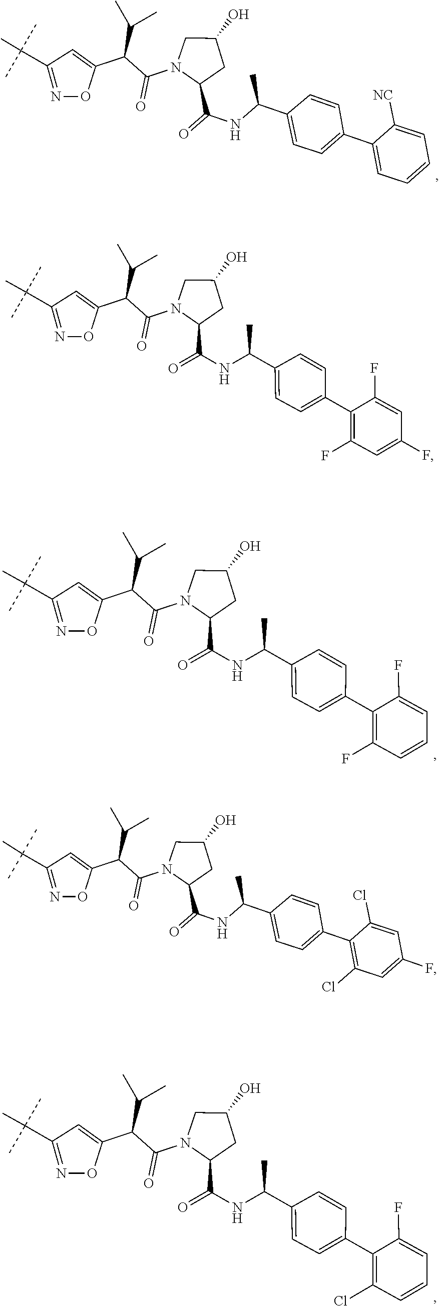

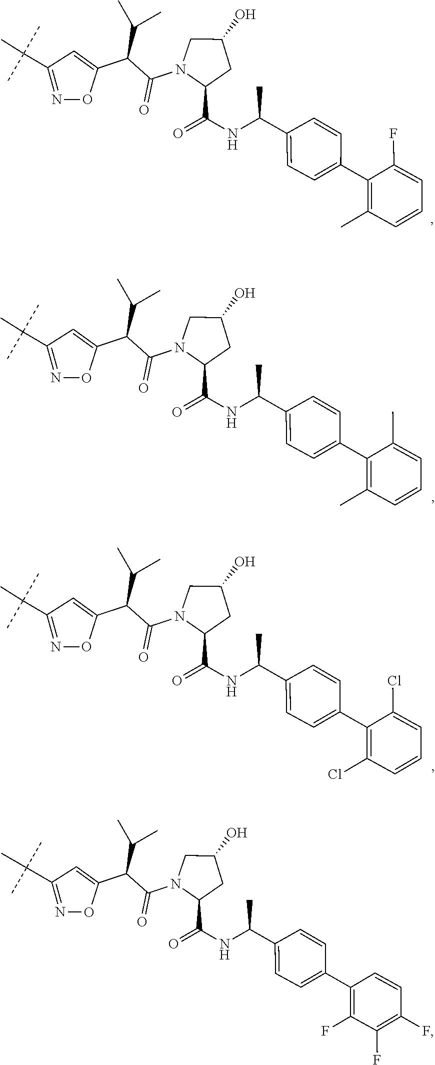

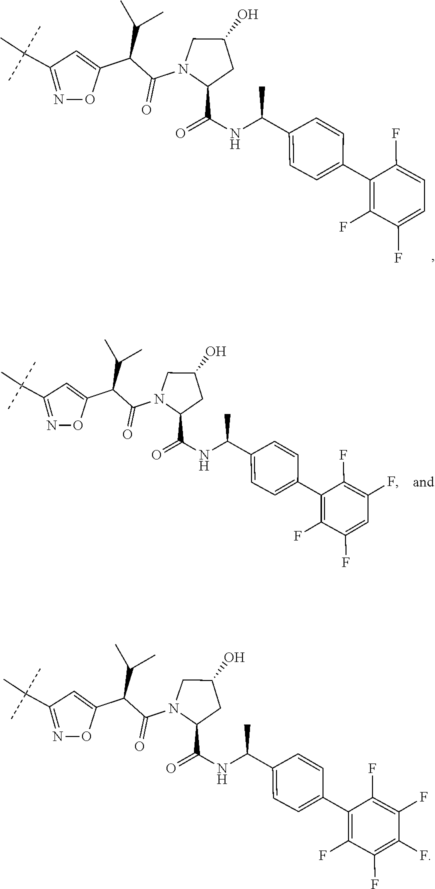

In embodiments, the target protein is a RAS protein. RAS proteins can include H-Ras, N-Ras, or K-Ras protein, including mutants thereof. In embodiments, the protein binding ligant is a pan-RAS inhibitor targeting two or more H-RAS, N-RAS, or K-RAS mutants.

In embodiments, the RAS protein is a KRAS mutant.

In embodiments, the KRAS mutant is selected from the group consisting of G12C, G12D, G12R, G12S, G12V, G13, and Q61H.

In embodiments, the protein binding ligand is a pan-KRAS inhibitor targeting two or more KRAS mutants.

In embodiments, the protein binding ligand is a KRAS mutant ligand.

In an aspect, the present application provides conjugates of Formula Q or a pharamceutically acceptable salt thereof:

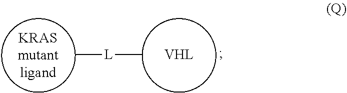

wherein the KRAS mutant ligand is capable of binding and/or inhibiting to a KRAS mutant protein; L is a bivalent linker that connects KRAS mutant ligand to a Von Hippel-Lindau (VHL) moiety; wherein VHL binds to a ubiquitin ligase; and wherein the VHL ligand is a structure according to (i) or (ii):

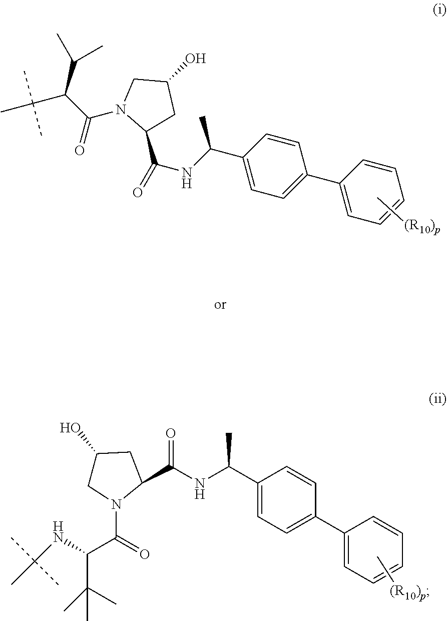

wherein p is an integer from 0 to 5; and
each R¹⁰ is independently selected from alkyl, —OH, cyano, and halogen.

In an embodiment, each R¹⁰ is fluorine. In an embodiment, each R¹⁰ is fluorine and p is 1 to 5. In an embodiment, R¹⁰ is chlorine. In an embodiment, each R¹⁰ is independently selected from chlorine and fluorine.

In an embodiment, L is: bond, NH, S, O, C(O), C(O)O, OC(O), NHC(O), C(O)NH, NHC(O)NH, NHC(NH)NH, C(S), substituted or unsubstituted alkylene, substituted or unsubstituted heteroalkylene, substituted or unsubstituted cycloalkylene, substituted or unsubstituted spirocycloalkylene, substituted or unsubstituted heterocycloalkylene, substituted or unsubstituted spiroheterocycloalkylene, substituted or unsubstituted arylene, substituted or unsubstituted heteroarylene or combinations thereof.

In an embodiment, -L- is a structure according to formula (L0):

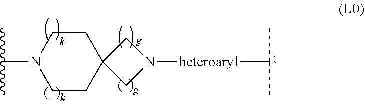

wherein each k is independently 0 or 1; and,
each g is independently 1 or 2.

In an embodiment, the target protein is a RAS protein.

In an embodiment, the RAS protein is a KRAS mutant.

In an embodiment, the KRAS mutant is selected from the group consisting of G12C, G12D, G12R, G12S, G12V, G13, and Q61H.

In an embodiment, the conjugate has a structure or pharmaceutically acceptable salt thereof selected from:

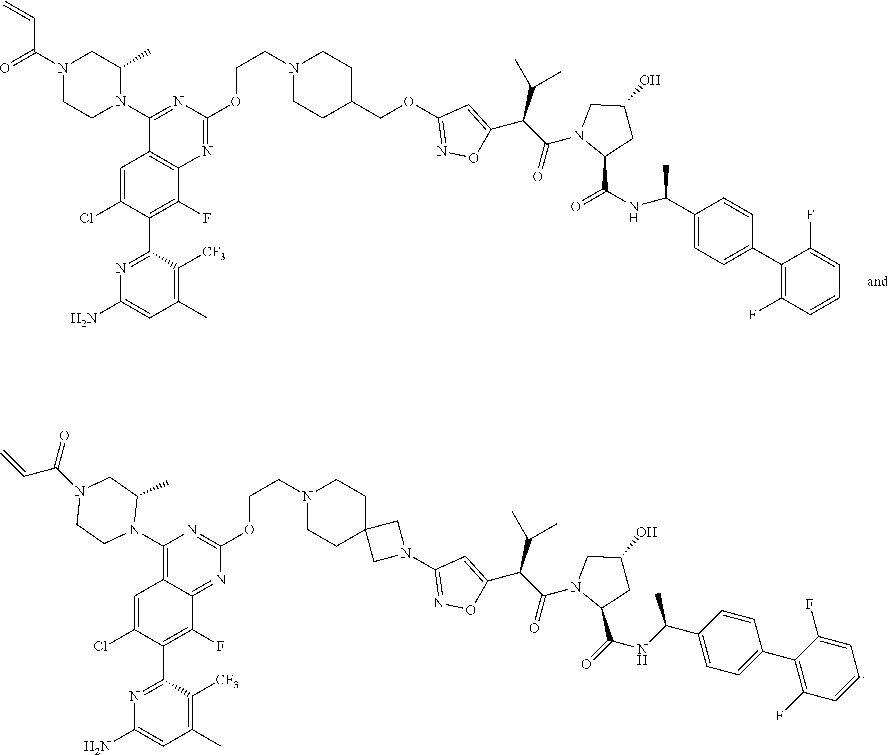

The compounds may exist in any number of combinations. The conjugates contemplated by the present application include any linker presented herein in combination with a VHL moiety, a UBM, or the like. In embodiments, the KRAS mutant ligand may be in combination with any of the linkers contemplated herein. In embodiments, the KRAS mutant ligand may be in combination with any of the VHL moieties contemplated herein. In embodiments, the KRAS mutant ligand may be in combination with a UBM contemplated herein. In embodiments, the G12D inhibitor or “G12D” may be replaced with a G12C inhibitor “G12C.”

The compounds disclosed herein can exist as salts. The present embodiments include such salts, which can be pharmaceutically acceptable salts. Examples of applicable salt forms include hydrochlorides, hydrobromides, sulfates, methanesulfonates, nitrates, maleates, acetates, citrates, fumarates, tartrates (eg (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds disclosed herein contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds disclosed herein contain both basic and acidic functionalities that allow the compounds to be converted into either base or acid addition salts.

Other salts include acid or base salts of the compounds used in the methods of the present embodiments. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts include salts of the active compounds which are prepared with relatively nontoxic acids or bases, depending on the particular substituents found on the compounds described herein. When compounds disclosed herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds disclosed herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds disclosed herein contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds disclosed herein can exist in unsolvated forms as well as solvated forms, including hydrated forms. In general, the solvated forms are equivalent to unsolvated forms and are encompassed within the scope of the present embodiments. Certain compounds disclosed herein may exist in multiple crystalline or amorphous forms. In general, all physical forms are equivalent for the uses contemplated by the present embodiments and are intended to be within the scope of the present embodiments.

Certain compounds disclosed herein possess asymmetric carbon atoms (optical centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present embodiments. The compounds disclosed herein do not include those which are known in art to be too unstable to synthesize and/or isolate. The present embodiments are meant to include compounds in racemic and optically pure forms. Optically active (R)- and (S)-, or (D)- and (L)-isomers may be prepared using chiral synthons or chiral reagents, or resolved using conventional techniques. The compounds disclosed herein can be provided as a mixture of atropisomers or can be pure atropisomers.

Isomers include compounds having the same number and kind of atoms, and hence the same molecular weight, but differing in respect to the structural arrangement or configuration of the atoms.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the embodiments.

Unless otherwise stated, the compounds disclosed herein may also contain unnatural proportions of atomic isotopes at one or more of the atoms that constitute such compounds. For example, the compounds disclosed herein may be labeled with radioactive or stable isotopes, such as for example deuterium (²H), tritium (³H), iodine-125 (¹²⁵I), fluorine-18 (¹⁸F), nitrogen-15 (¹⁵N), oxygen-17 (¹⁷O), oxygen-18 (¹⁸O), carbon-13 (¹³C), or carbon-14 (¹⁴C). All isotopic variations of the compounds disclosed herein, whether radioactive or not, are encompassed within the scope of the present embodiments.

In addition to salt forms, the present embodiments provide compounds, which are in a prodrug form. Prodrugs of the compounds described herein are those compounds that readily undergo chemical changes under physiological conditions to provide the compounds disclosed herein. Additionally, prodrugs can be converted to the compounds disclosed herein by chemical or biochemical methods in an ex vivo environment. For example, prodrugs can be slowly converted to the compounds disclosed herein when placed in a transdermal patch reservoir with a suitable enzyme or chemical reagent.

Compounds disclosed herein can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, vol. 1-21; R. C. LaRock, Comprehensive Organic Transformations, 2nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds.) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, vol. 1-40. The following synthetic reaction schemes are merely illustrative of some methods by which the compounds disclosed herein can be synthesized, and various modifications to these synthetic reaction schemes can be made and will be suggested to one skilled in the art having referred to the disclosure contained herein.

For illustrative purposes, reaction Schemes below provide routes for synthesizing the compounds disclosed herein as well as key intermediates. For a more detailed description of the individual reaction steps, see the Examples section below. Those skilled in the art will appreciate that other synthetic routes may be used. Although some specific starting materials and reagents are depicted in the Schemes and discussed below, other starting materials and reagents can be substituted to provide a variety of derivatives or reaction conditions. In addition, many of the compounds prepared by the methods described below can be further modified in light of this disclosure using conventional chemistry well known to those skilled in the art.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, or, about 20° C.

Some compounds in following schemes are depicted with generalized substituents; however, one skilled in the art will immediately appreciate that the nature of the substituents can varied to afford the various compounds contemplated in the present embodiments. Moreover, the reaction conditions are exemplary and alternative conditions are well known. The reaction sequences in the following examples are not meant to limit the scope of the embodiments as set forth in the claims.

### IV. Pharmaceutical Formulations

In some embodiments, pharmaceutical compositions comprise a conjugate of any one of the compounds disclosed herein and a pharmaceutically acceptable excipient.

In some embodiments, there is provided a pharmaceutical composition comprising a pharmaceutically effective amount of a conjugate of Formula (A) or a pharmaceutically acceptable salt thereof, and a pharmaceutically acceptable excipient.

In some embodiments, the pharmaceutical composition further comprises an additional therapeutic agent.

In some embodiments, the additional therapeutic agent is a chemotherapeutic agent. In some embodiments, the chemotherapeutic agent is an anti-microtubule agent, a platinum coordination complex, a alkylating agent, an antibiotic agent, a topoisomerase II inhibitor, a antimetabolite, a topoisomerase I inhibitor, a hormone or hormonal analogue, a signal transduction pathway inhibitor, a non-receptor tyrosine kinase angiogenesis inhibitor, a immunotherapeutic agent, a proapoptotic agent, an inhibitor of LDH-A, an inhibitor of fatty acid biosynthesis, a cell cycle signalling inhibitor, a HDAC inhibitor, a proteasome inhibitor, or an inhibitor of cancer metabolism. In some embodiments, the chemotherapeutic agent is cisplatin, carboplatin, doxorubicin, ionizing radiation, docetaxel or paclitaxel.

The compounds disclosed herein can be prepared and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. The compounds disclosed herein can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds disclosed herein can be administered transdermally. The compounds disclosed herein can also be administered by in intraocular, intravaginal, and intrarectal routes including suppositories, insufflation, powders and aerosol formulations (for examples of steroid inhalants, see Rohatagi, J Clin. Pharmacol. 35:1187-1193, 1995; Tjwa, Ann. Allergy Asthma Immunol. 75:107-111, 1995). Accordingly, the present embodiments also provide pharmaceutical compositions including one or more pharmaceutically acceptable carriers and/or excipients and either a compound of Formula I, or a pharmaceutically acceptable salt of a compound of Formula I.

For preparing pharmaceutical compositions from the conjugates disclosed herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, surfactants, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties and additional excipients as required in suitable proportions and compacted in the shape and size desired.

The powders, capsules and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other exceipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers including, but not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations can also be used orally using, for example, push-fit capsules made of gelatin, as well as soft, sealed capsules made of gelatin and a coating such as glycerol or sorbitol. Push-fit capsules can contain the compounds disclosed herein mixed with a filler or binders such as lactose or starches, lubricants such as talc or magnesium stearate, and, optionally, stabilizers. In soft capsules, the compounds disclosed herein may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compounds disclosed herein in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compounds disclosed herein can be delivered by transdermally, by a topical route, formulated as applicator sticks, solutions, suspensions, emulsions, gels, creams, ointments, pastes, jellies, paints, powders, and aerosols.

The compounds disclosed herein can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The pharmaceutical formulations of the compounds disclosed herein can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

The pharmaceutical formulations of the compounds disclosed herein can be provided as a salt and can be formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

In some embodiments, the formulations of the compounds disclosed herein can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the GR modulator into the target cells in vivo. (See, e.g., A1-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J Pharm. Sci. 84:1144-1146; Rohatagi (1995) Pharmazie 50:610-613; Brophy (1983) Eur. J Clin. Pharmacol. 24:103-108; the latest Remington's, supra). The state of the art allows the clinician to determine the dosage regimen for each individual patient, GR and/or MR modulator and disease or condition treated.

Single or multiple administrations of the compounds disclosed herein formulations can be administered depending on the dosage and frequency as required and tolerated by the patient. The formulations should provide a sufficient quantity of active agent to effectively treat the disease state. Thus, in one embodiment, the pharmaceutical formulations for oral administration of the compounds disclosed herein is in a daily amount of between about 0.5 to about 30 mg per kilogram of body weight per day. In an alternative embodiment, dosages are from about 1 mg to about 20 mg per kg of body weight per patient per day are used. Lower dosages can be used, particularly when the drug is administered to an anatomically secluded site, such as the cerebral spinal fluid (CSF) space, in contrast to administration orally, into the blood stream, into a body cavity or into a lumen of an organ. Substantially higher dosages can be used in topical administration. Actual methods for preparing formulations including the compounds disclosed herein for parenteral administration are known or apparent to those skilled in the art and are described in more detail in such publications as Remington's, supra. See also Nieman, In “Receptor Mediated Antisteroid Action,” Agarwal, et al., eds., De Gruyter, New York (1987).

The compounds described herein can be used in combination with one another, with other active agents known to be useful in modulating a glucocorticoid receptor, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In some embodiments, the active agents can be formulated separately. In some embodiments, the active and/or adjunctive agents may be linked or conjugated to one another.

After a pharmaceutical composition including a compound disclosed herein has been formulated in one or more acceptable carriers, it can be placed in an appropriate container and labeled for treatment of an indicated condition. For administration of the compounds of Formula I, such labeling would include, e.g., instructions concerning the amount, frequency and method of administration.

In some embodiments, the compositions disclosed herein are useful for parenteral administration, such as intravenous (IV) administration or administration into a body cavity or lumen of an organ. The formulations for administration will commonly comprise a solution of the compositions disclosed herein dissolved in one or more pharmaceutically acceptable carriers. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, tonicity adjusting agents, e.g., sodium acetate, sodium chloride, potassium chloride, calcium chloride, sodium lactate and the like. The concentration of the compositions in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in anontoxic parenterally-acceptable diluent or solvent, such as a solution of 1,3-butanediol.

In some embodiments, the formulations of the compositions disclosed herein can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions disclosed herein into the target cells in vivo. (See, e.g., A1-Muhammed, J Microencapsul. 13:293-306, 1996; Chonn, Curr. Opin. Biotechnol. 6:698-708, 1995; Ostro, Am. J Hosp. Pharm. 46:1576-1587, 1989).

### V. Methods

In some embodiments, there is provided a method of treating a disorder or condition in a subject, the method comprising administering to the human a therapeutically effective amount of a compound disclosed herein, or a pharmaceutically acceptable salt thereof, or a pharmaceutical composition as disclosed herein.

As used herein, the term “KRAS” refers to Kirsten rat carcoma virus. The KRAS or “K-Ras” protein is a GTPase, a class of enzymes that convert the nucleotide guanosine triphosphate into guanosine diphosphate. KRAS is an intregral part of numerous signal transduction pathways.

As used herein, the “KRAS G12D” refers to the G12D mutation. Specifically, the amino acid position 12 of the KRAS protein is an cysteine instead of a glycine (wild-type). The present application contemplates ligands that are KRAS G12D inhibitors. KRAS G12D inhibitors specifically bind to the KRAS G12D.

Example KRAS G12D inhibitors adaptable into a PROTAC degrader include those disclosed in WO/2022/105859, WO/2022/105855, WO/2022/105857, WO/2022/098625, WO/2022/066646, WO/2022/042630, WO/2022/031678, WO/2022/015375, WO/2022/002102, WO/2021/248079, WO/2021/248095, WO/2021/248082, WO/2021/248083, WO/2021/248090, WO/2021/215544, WO/2021/107160, WO/2021/106231, WO/2021/081212, and WO/2021/081212, all of which are incorporated herein by reference in their entirety.

As used herein, the “KRAS G12C” refers to the G12C mutation. Specifically, the amino acid position 12 of the KRAS protein is an aspartic acid instead of a glycine (wild-type). In other aspects of the application, ligands that are KRAS G12C inhibitors are contemplated. KRAS G12C inhibitors specifically bind to the KRAS G12C. Example KRAS G12C inhibitors adaptable into a PROTAC degrader include those disclosed in WO/2022/119748, WO/2022/111513, WO/2022/115439, WO/2022/111527, WO/2022/111521, WO/2022/109485, WO/2022/109487, WO/2022/093856, WO/2022/087371, WO/2022/087624, WO/2022/087375, WO/2022/083569, WO/2022/081655, WO/2022/063297, WO/2022/037560, WO/2022/028492, WO/2021/259331, WO/2021/249563, WO/2021/252339, WO/2021/244603, WO/2021/248079, WO/2021/248095, WO/2021/248082, WO/2021/248083, WO/2021/248090, WO/2021/218110, WO/2021/219090, WO/2021/219091, WO/2021/216770, WO/2021/190467, WO/2021/168193, WO/2021/155716, WO/2021/143693, WO/2021/141628, WO/2021/139678, WO/2021/129824, WO/2021/129820, WO/2021/118877, WO/2021/113595, WO/2021/104431, WO/2021/098859, WO/2021/093758, WO/2021/088938, WO/2021/086833, WO/2021/078285, WO/2021/081212, WO/2021/068898, WO/2021/063346, WO/2021/058018, WO/2021/055728, WO/2021/043322, WO/2021/037018, WO/2021/027911, WO/2021/027943, WO/2020/259432, WO/2020/259573, WO/2020/259513, WO/2020/239077, WO/2020/239123, WO/2020/233592, WO/2020/236940, WO/2020/156285, WO/2020/146613, WO/2020/113071, WO/2020/106640, WO/2020/101736, WO/2020/086739, WO/2020/081282, WO/2020/050890, WO/2020/047192, WO/2020/028706, WO/2020/027083, WO/2020/027084, WO/2019/241157, WO/2019/232419, WO/2019/217307, WO/2019/217691, WO/2019/213516, WO/2019/213526, WO/2019/141250, WO/2019/110751, WO/2019/099524, WO/2019/051291, WO/2018/218069, WO/2018/218070, WO/2018/217651, WO/2018/218071, WO/2018/206539, WO/2018/143315, WO/2018/140513, WO/2018/140514, WO/2018/140598, WO/2018/140599, WO/2018/140600, WO/2018/140512, WO/2018/119183, WO/2018/068017, WO/2018/064510, WO/2017/201161, WO/2017/087528, WO/2017/058915, WO/2017/058902, WO/2017/058768, WO/2017/058728, WO/2017/058805, WO/2017/058807, WO/2017/058792, WO/2017/015562, WO/2016/168540, WO/2016/164675, WO/2016/049524, WO/2015/054572, WO/2014/152588, and WO/2014/143659, all of which are incorporated herein by reference in their entirety.

In some embodiments, there is provided a method for inhibiting KRAS G12D activity in a cell, comprising contacting the cell in which inhibition of KRAS G12D activity is desired with an effective amount of a compound disclosed herein or a pharmaceutically acceptable salt thereof.

In some embodiments, there is provided a method for inhibiting KRAS G12D activity in a cell, comprising contacting the cell in which inhibition of KRAS G12D activity is desired with the pharmaceutical composition disclosed herein.

In some embodiments, there is provided a method for treating a KRAS G12D-associated cancer comprising administering to a patient in need thereof a therapeutically effective amount of a compound disclosed herein, or a pharmaceutically acceptable salt thereof.

In some embodiments, there is provided a method for treating a KRAS G12D-associated cancer comprising administering to a patient in need thereof the pharmaceutical composition disclosed herein.

In some embodiments, there is provided a method of treating a subject having cancer, the cancer characterized by the presence of a KRAS G12D mutation, the method comprising administering to the human a therapeutically effective amount of a compound of any one of Formula (I) or Formula (II), or a pharmaceutically acceptable salt thereof, or a a pharmaceutical composition as disclosed herein.

In some embodiments, there is provided a method for manufacturing a medicament for treating a subject having cancer, the cancer characterized by the presence of a KRAS G12D mutation, the compound comprising Formula (I) or Formula (II), or a pharmaceutically acceptable salt thereof, or a pharmaceutical composition.

In some embodiments, there is provided a use of a compound of Formula (I) or Formula (II), or a pharmaceutically acceptable salt thereof, or a a pharmaceutical composition as disclosed herein, for the manufacture of a medicament for the treatment in a human having cancer, the cancer characterized by the presence of a KRAS G12D mutation.

In some embodiments, there are provided compounds of Formula (I) or Formula (II), or a pharmaceutically acceptable salt thereof, or a a pharmaceutical composition as disclosed herein, for use in the treatment of a subject having cancer, the cancer characterized by the presence of a KRAS G12D mutation.

In some embodiments, there is provided a method for treating cancer in a patient in need thereof, the method comprising (a) determining that the cancer is associated with a KRAS G12D mutation (e.g., a KRAS G12D-associated cancer); and (b) administering to the patient a therapeutically effective amount of a compound disclosed herein.

In some embodiments, there is provided a method for treating cancer in a patient in need thereof, the method comprising (a) determining that the cancer is associated with a KRas G12D mutation (e.g., a KRAS G12D-associated cancer); and (b) administering to the patient the pharmaceutical composition disclosed herein.

In some embodiments, the cancer is Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial 'carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; or Adrenal glands: neuroblastoma.

In some embodiments, the cancer is non-small cell lung cancer, small cell lung cancer, colorectal cancer, rectal cancer, or pancreatic cancer.

In certain embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The compounds of Formula (I) or Formula (II), or a pharmaceutically acceptable salt thereof, can be inhibitors of KRAS G12D. For example, the inhibition constant (Ki) of the compounds disclosed herein can be less than about 50 μM, or less than about 40, 30, 20, 10, 9, 8, 7, 6, 5, 4, 3, 2, or less than about 1 μM. The inhibition constant (Ki) of the compounds disclosed herein can be less than about 1,000 nM, or less than about 900, 800, 700, 600, 500, 400, 300, 200, 100, 90, 80, 70, 60, 50, 40, 30, 20, 10, 9, 8, 7, 6, 5, 4, 3, 2, or less than about 1 nM. The inhibition constant (Ki) of the compounds disclosed herein can be less than about 1 nM, or less than about 0.9, 0.8, 0.7, 0.6, 0.5, 0.4, 0.3, 0.2, or less than about 0.1 nM.

The compounds of Formula (I) or Formula (II), or a pharmaceutically acceptable salt thereof, can be selective inhibitors of KRAS G12D. For example, KRAS G12D inhibition constant (IC50) of the compounds disclosed herein can be at least 2-fold less than the inhibition constant of one or more of KRAS wild-type, or NRAS, or HRAS, or at least 3, 4, 5, 6, 7, 8, 9, 10, 20, 30, 40, 50, 60, 70, 80, 90 or 100-fold less. The KRAS g12D inhibition constant (Ki) of the compounds disclosed herein can also be at least 100-fold less than the inhibition constant of one or more of KRAS wild-type, or NRAS, or HRAS, or at least 200, 300, 400, 500, 600, 700, 800, 900, 1000, or 10,000-fold less.

### A. Cancer Combination Therapies

The compounds disclosed herein or salts thereof may be employed alone or in combination with other agents for treatment. For example, the second agent of the pharmaceutical combination formulation or dosing regimen may have complementary activities to the compounds disclosed herein such that they do not adversely affect each other. The compounds may be administered together in a unitary pharmaceutical composition or separately. In one embodiment a compound or a pharmaceutically acceptable salt can be co-administered with a cytotoxic agent to treat proliferative diseases and cancer.

The term “co-administering” refers to either simultaneous administration, or any manner of separate sequential administration, of a compound disclosed herein or a salt thereof, and a further active pharmaceutical ingredient or ingredients, including cytotoxic agents and radiation treatment. If the administration is not simultaneous, the compounds are administered in a close time proximity to each other. Furthermore, it does not matter if the compounds are administered in the same dosage form, e.g. one compound may be administered topically and another compound may be administered orally.

Those additional agents may be administered separately from an inventive compound-containing composition, as part of a multiple dosage regimen. Alternatively, those agents may be part of a single dosage form, mixed together with a compound disclosed herein, in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with embodiments herein. For example, a compound disclosed herein may be administered with another therapeutic agent simultaneously or sequentially in separate unit dosage forms or together in a single unit dosage form. Accordingly, the present embodiments provide a single unit dosage form comprising a compound of Formula I, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. In certain embodiments, compositions disclosed herein are formulated such that a dosage of between 0.01-100 mg/kg body weight/day of an inventive can be administered.

Typically, any agent that has activity against a disease or condition being treated may be co-administered. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the disease involved.

In one embodiment, the treatment method includes the co-administration of a compound disclosed herein or a pharmaceutically acceptable salt thereof and at least one cytotoxic agent. The term “cytotoxic agent” as used herein refers to a substance that inhibits or prevents a cellular function and/or causes cell death or destruction. Cytotoxic agents include, but are not limited to, radioactive isotopes (e.g., At²r, I¹³¹, I¹²⁵, Y⁹⁰, Re¹⁸⁶, Re¹⁸⁸, Sm¹⁵³, Bi²¹², p³², Pb²¹² and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof.

Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signalling inhibitors; HDAC inhibitors, proteasome inhibitors; and inhibitors of cancer metabolism.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG(geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®., Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5□-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX©; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds disclosed herein include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgGi λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or famesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R₁₁₅₇₇); orafenib, ABT510; Bcl-2 inhibitor such as oblimersen sodium (GENASENSE®); pixantrone; famesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

In certain embodiments, chemotherapeutic agents include, but are not limited to, doxorubicin, dexamethasone, vincristine, cyclophosphamide, fluorouracil, topotecan, interferons, platinum derivatives, taxanes (e.g., paclitaxel, docetaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, trimetrexate, metoprine, cyclosporine, daunorubicin, teniposide, amphotericin, alkylating agents (e.g., chlorambucil), 5-fluorouracil, campthothecin, cisplatin, metronidazole, and imatinib mesylate, among others. In other embodiments, a compound disclosed herein is administered in combination with a biologic agent, such as bevacizumab or panitumumab.

In certain embodiments, compounds disclosed herein, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG live, bevacuzimab, fluorouracil, bexarotene, bleomycin, bortezomib, busulfan, calusterone, capecitabine, camptothecin, carboplatin, carmustine, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, elotinib, estramustine, etoposide phosphate, etoposide, exemestane, filgrastim, floxuridine, fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

Chemotherapeutic agents also include treatments for Alzheimer's Disease such as donepezil hydrochloride and rivastigmine; treatments for Parkinson's Disease such as L-DOPA/carbidopa, entacapone, ropinrole, pramipexole, bromocriptine, pergolide, trihexephendyl, and amantadine; agents for treating multiple sclerosis (MS) such as beta interferon (e.g., Avonex® and Rebif®), glatiramer acetate, and mitoxantrone; treatments for asthma such as albuterol and montelukast sodium; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

Additionally, chemotherapeutic agents include pharmaceutically acceptable salts, acids or derivatives of any of chemotherapeutic agents, described herein, as well as combinations of two or more of them.

### VI. EXAMPLES

### Abbreviations

ACN—acetonitrile
AC₂O—acetyl acetate
BINAP—(+/−)-2,2′-bis(diphenylphosphino)-l,r-binaphthyl
Boc₂O—di-tert-butyl dicarbonate
BOP—(benzotriazol-1-yloxy)tris(dimethylamino)phosphonium hexafluorophosphate
DBU—1,8-diazabicyclo[5.4.0]undec-7-ene
DCE—1,2-dichloroethane
DCM—dichloromethane
DIEA or DIPEA—N,N-diisopropylethylamine
DMA—N,N-dimethylacetamide
DMAc—N,N-dimethylacetamide
DMAP—4-dimethylaminopyridine
DMF—N,N-dimethylformamide
DMSO—dimethyl sulfoxide
EA—ethyl acetate
EtOAc—ethyl acetate
EtOH—ethanol
HATU—2-(7-azabenzotriazol-1-yl)-N,N,N′,N′-tetramethyluronium hexafluorophosphate
HFIP—hexafluoroisopropanol
HOAc—acetic acid
iPrOAc—isopropyl acetate
KF—potassium fluoride
KOAc—potassium acetate
LDA—lithium diisopropylamide
LiHMDS—lithium bis(trimethylsilyl)amide
mCPBA—3-chloroperoxybenzoic acid
MeCN—acetonitrile
MeI—iodomethane
MeOH—methanol
MeONa—sodium methoxide or sodium methanolate
MTBE—methyl tert-butyl ether
MW—microwave
NaBH(OAc)₃—sodium triacetoxyborohydride
NIS—N-iodosuccinimide
P(Cy)₃ or PCy₃— tricyclohexylphosphine
P(t-Bu)₃HBF₄—tri-tert-buty lphosphonium tetrafluoroborate
Pd/C—palladium on carbon
Pd₂(dba)₃—tris(dibenzylideneacetone)dipalladium(O)
Pd₂(dba)₃CHCl₃—tris(dibenzylidenacetone)dipalladium(0) chloroform
Pd(dppf)Cl₂·CH₂Cl₂—[1,1′-bis(diphenylphosphino)ferrocene]dichloropalladium(II) or
dichloro[1.1′-bis(diphenylphosphino)ferrocene]palladium(II), complexed with
dichloromethane
Pd(PPh₃)₄—tetrakis(triphenylphosphine)palladium(0)
Pd(PPh₃)₂Cl₂—bis(triphenylphosphine)palladium(II) dichloride
PE—petroleum ether
PMBCl—4-methoxybenzylchloride
pTsA—p-toluenesulfbnic acid
r.t.—room temperature
Sn₂(n-Bu)₆—hexabutylditin
TBSCl—tert-butyldimethylsilyl chloride or tert-butyldimethylchlorosilane
[Rh(COD)Cl]₂—chloro(1,5-cyclooctadiene)rhodium(I) dimer
TEA—triethylamine
TFA—trifluoroacetic acid or 2,2,2-trifluoroacetic acid
THF—tetrahydrofuran
THP—tetrahydropyran
TsOH—p-toluenesulfonic acid

### A. Synthetic Procedures

### General Procedure

The compounds of Formula (A) may be prepared from commercially available reagents using the synthetic methods and reaction schemes herein, or using other reagents and conventional methods well known to those skilled in the art. For instance, compounds of the present invention may be prepared according to the general reaction schemes set forth below.

Synthesis of Intermediate 1: tert-butyl 3-[7-[6-[bis[(4-methoxyphenyl)methyl]amino]-4-methyl-3-(trifluoromethyl)-2-pyridyl]-6-chloro-2,8-difluoro-quinazolin-4-yl]-3,8-diazabicyclo[3.2.1]octane-8-carboxylate

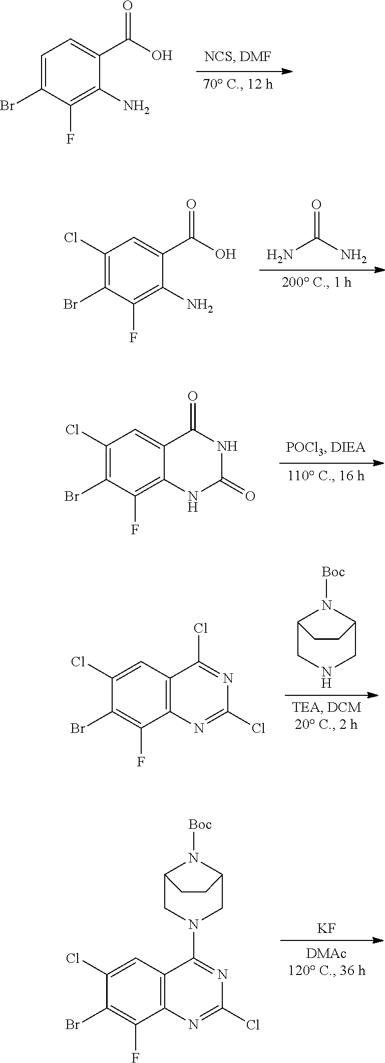

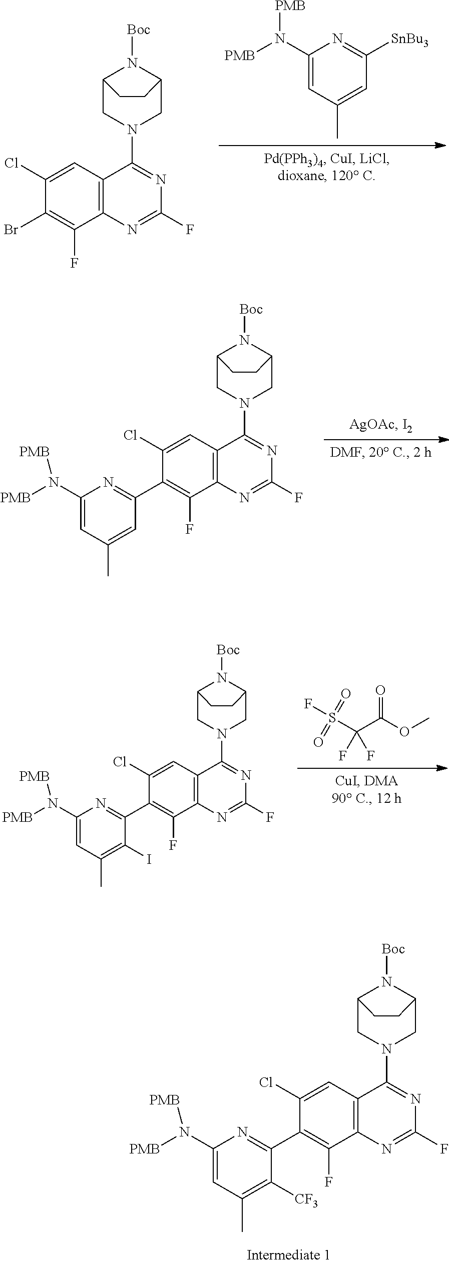

Step 1: 2-amino-4-bromo-5-chloro-3-fluoro-benzoic acid

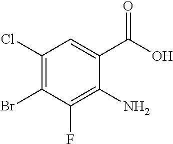 [SMILES: Nc1c(C(=O)O)cc(Cl)c(Br)c1F]

To a solution of 2-amino-4-bromo-3-fluoro-benzoic acid (25 g, 106.83 mmol, 1 eq) in N,N-dimethylformamide (400 mL) was added N-chlorosuccinimide (15.69 g, 117.51 mmol, 1.1 eq). The mixture was stirred at 70° C. for 12 hours. The mixture was cooled to 25° C., and then poured into water (2000 mL). The resultant mixture was filtered and the filter cake was collected, dried under reduced pressure to get the crude product (28 g, 104.30 mmol, 97% yield) as a yellow solid, which was used for next step directly. ¹H NMR: (400 MHz, DMSO-d₆) δ: 13.48 (s, 1H), 7.68 (s, 1H), 6.90 (s, 2H).

Step 2: 7-bromo-6-chloro-8-fluoro-1H-quinazoline-2,4-dione

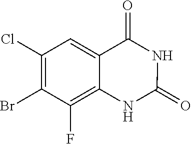 [SMILES: O=c1[nH]c(=O)c2cc(Cl)c(Br)c(F)c2[nH]1]

A mixture of 2-amino-4-bromo-5-chloro-3-fluoro-benzoic acid (29 g, 108.0 mmol, 1 eq) in urea (64.87 g, 1.08 mol, 10 eq) was stirred at 200° C. for 1 hour. LCMS showed that the desired mass was detected. The mixture was cooled to 25° C., diluted with water (800 mL) and stirred at 25° C. for 1 hour. The mixture was filtered and the solid was dried in vacuum to give the crude product (31 g, 105.63 mmol, 97% yield) as a brown solid, which was used for next step directly. LCMS (ESI, m/z): 293.9 [M+1]⁺. ¹H NMR (400 MHz, DMSO-d₆) δ: 7.24 (s, 1H).

Step 3: 7-bromo-2,4,6-trichloro-8-fluoro-quinazoline

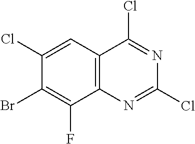 [SMILES: Fc1c(Br)c(Cl)cc2c(Cl)nc(Cl)nc12]

To a solution of 7-bromo-6-chloro-8-fluoro-1H-quinazoline-2,4-dione (31 g, 105.6 mmol, 1 eq) in phosphoryl chloride (360 mL) was added N,N-diisopropylethylamine (310.0 mmol, 54 mL, 2.94 eq), the mixture was stirred at 110° C. for 16 hours. LCMS showed that the reactant was consumed completely. The mixture was concentrated under reduced pressure to give the crude product. The crude product was purified by silica gel column chromatography eluted by petroleum ether/tetrahydrofuran=40/1 to give the product (24 g, 72.65 mmol, 68% yield) as a yellow solid. ¹H NMR: (400 MHz, DMSO-d₆) δ: 8.03 (s, 1H).

Step 4: tert-butyl 3-(7-bromo-2,6-dichloro-8-fluoro-quinazolin-4-yl)-3,8-diazabicyclo[3.2.1]octane-8-carboxylate

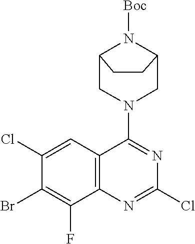 [SMILES: CC(C)(C)OC(=O)N1C2CCC1CN(c1nc(Cl)nc3c(F)c(Br)c(Cl)cc13)C2]

To a solution of tert-butyl 3,8-diazabicyclo[3.2.1]octane-8-carboxylate (1.93 g, 9.08 mmol, 1 eq) in dichloromethane (40 mL) was added triethylamine (2.76 g, 27.24 mmol, 3.8 mL, 3 eq) and 7-bromo-2,4,6-trichloro-8-fluoro-quinazoline (3 g, 9.08 mmol, 1 eq). The mixture was stirred at 20° C. for 2 hours. LCMS showed the starting material was consumed completely and one main peak with desired mass was detected. Water (50 mL) was added before the mixture was extracted by dichloromethane (30 mL×3). The combined organic layers were washed with brine (30 mL×3), dried over anhydrous sodium sulfate, filtered and concentrated under reduced pressure to give a residue. The residue was purified by column chromatography (silicon dioxide, petroleum ether/ethyl acetate=30/1 to 10/1) to give the product (3.6 g, 7.11 mmol, 78% yield) as a white solid. LCMS (ESI, m/z): 507.2 [M+1]⁺. ¹H NMR: (400 MHz, CDCl3) δ: 7.75 (d, J=2.0 Hz, 1H), 4.40 (s, 4H), 3.80-3.52 (m, 2H), 2.02-1.92 (m, 2H), 1.80-1.68 (m, 2H), 1.53 (s, 9H).

Step 5: tert-butyl 3-(7-bromo-6-chloro-2,8-difluoro-quinazolin-4-yl)-3,8-diazabicyclo[3.2.1]octane-8-carboxylate

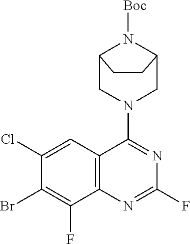 [SMILES: CC(C)(C)OC(=O)N1C2CCC1CN(c1nc(F)nc3c(F)c(Br)c(Cl)cc13)C2]

To a solution of tert-butyl 3-(7-bromo-2,6-dichloro-8-fluoro-quinazolin-4-yl)-3,8-diazabicyclo[3.2.1]octane-8-carboxylate (3.5 g, 6.91 mmol, 1 eq) in N,N-dimethylacetamide (50 mL) was added potassium fluoride (12.05 g, 207.43 mmol, 4.9 mL, 30 eq). The mixture was stirred at 120° C. for 36 hours. LCMS showed the starting material was consumed completely and one main peak with desired mass was detected. Water (500 mL) was added before the mixture was extracted by ethyl acetate (250 mL×3). The combined organic layers were washed with brine (500 mL×2), dried over anhydrous sodium sulfate, filtered and concentrated under reduced pressure to give a residue. The residue was purified by column chromatography (silicon dioxide, petroleum ether/ethyl acetate=1/0 to 5/1) to give the product (1.9 g, 3.88 mmol, 56% yield) as a white solid. LCMS (ESI, m/z): 491.2 [M+1]⁺. ¹H NMR: (400 MHz, CDCl₃) δ: 7.80 (d, J=0.8 Hz, 1H), 4.52-4.29 (m, 4H), 3.85-3.53 (m, 2H), 2.02-1.86 (m, 2H), 1.81-1.67 (m, 2H), 1.53 (s, 9H).

Step 6: tert-butyl 3-[7-[6-[bis[(4-methoxyphenyl)methyl]amino]-4-methyl-2-pyridyl]-6-chloro-2,8-difluoro-quinazolin-4-yl]-3,8-diazabicyclo[3.2.1]octane-8-carboxylate

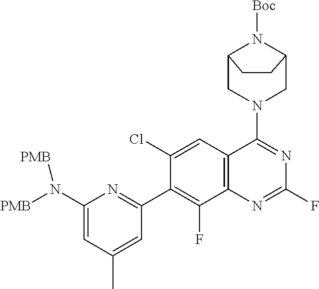

A mixture of tert-butyl 3-(7-bromo-6-chloro-2,8-difluoro-quinazolin-4-yl)-3,8-diazabicyclo[3.2.1]octane-8-carboxylate (1 g, 2.04 mmol, 1 eq), N,N-bis[(4-methoxyphenyl)methyl]-4-methyl-6-tributylstannyl-pyridin-2-amine (2.60 g, 4.08 mmol, 2 eq), tetrakis[triphenylphosphine]palladium(0) (471 mg, 0.40 mmol, 0.2 eq), cuprous iodide (116 mg, 0.61 mmol, 0.3 eq) and lithium chloride (216 mg, 5.10 mmol, 2.5 eq) in dioxane (20 mL) was degassed and purged with nitrogen for 3 times, and then the mixture was stirred at 120° C. for 12 hours under nitrogen atmosphere. LCMS showed the starting material was consumed completely and one main peak with desired mass was detected. The reaction mixture was partitioned between ethyl acetate (150 mL) and water (100 mL). Then the organic phase was separated, washed with brine (100 mL×3), dried over anhydrous sodium sulfate, filtered and concentrated under reduced pressure to give a residue. The residue was purified by column chromatography (silicon dioxide, petroleum ether/ethyl acetate=15/1 to 8/1) to afford the product (1.2 g, 1.58 mmol, 77% yield) as a yellow solid. LCMS (ESI, m/z): 757.5[M+1]⁺. ¹H NMR: (400 MHz, CDCl₃) δ: 7.75 (d, J=1.2 Hz, 1H), 7.18 (d, J=8.8 Hz, 4H), 6.89-6.82 (m, 4H), 6.59 (s, 1H), 6.38 (s, 1H), 4.69 (s, 4H), 4.48 (d, J=12.0 Hz, 2H), 4.43-4.30 (m, 2H), 3.80 (s, 6H), 3.75-3.56 (m, 2H), 2.28 (s, 3H), 2.00-1.92 (m, 2H), 1.76 (d, J=6.8 Hz, 2H), 1.53 (s, 9H).

Step 7: tert-butyl 3-[7-[6-[bis[(4-methoxyphenyl)methyl]amino]-3-iodo-4-methyl-2-pyridyl]-6-chloro-2,8-difluoro-quinazolin-4-yl]-3,8-diazabicyclo[3.2.1]octane-8-carboxylate

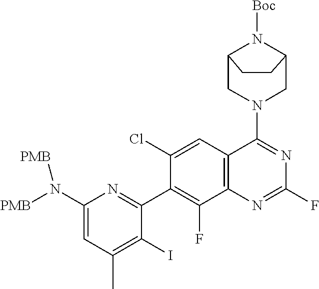

To a solution of tert-butyl 3-[7-[6-[bis[(4-methoxyphenyl)methyl]amino]-4-methyl-2-pyridyl]-6-chloro-2,8-difluoro-quinazolin-4-yl]-3,8-diazabicyclo[3.2.1]octane-8-carboxylate (500 mg, 0.66 mmol, 1 eq) in N,N-dimethylformamide (5 mL) was added iodine (502 mg, 1.98 mmol, 3 eq) and silver acetate (275 mg, 1.65 mmol, 2.5 eq). The mixture was stirred at 20° C. for 2 hours. LCMS showed the starting material was consumed completely and one main peak with desired mass was detected. The reaction mixture was partitioned between ethyl acetate (50 mL) and saturated sodium thiosulfate solution (50 mL). The organic phase was separated, washed with saturated sodium thiosulfate solution (50 mL×3) and brine (50 ml×3), dried over anhydrous sodium sulfate, filtered and concentrated under reduced pressure to give a residue. The residue was purified by prep-TLC (silicon dioxide, petroleum ether/ethyl acetate=3/1) to generate the product (510 mg, 0.57 mmol, 87% yield) as a yellow solid. LCMS (ESI, m/z): 883.4[M]⁺. ¹H NMR: (400 MHz, DMSO-d₆) δ: 8.09 (s, 1H), 7.15 (d, J=8.8 Hz, 4H), 6.86 (d, J=8.8 Hz, 4H), 6.76 (s, 1H), 4.69 (d, J=16.0 Hz, 2H), 4.51 (d, J=16.0 Hz, 3H), 4.37-4.20 (m, 3H), 3.80-3.69 (m, 7H), 3.62-3.51 (m, 1H), 2.32 (s, 3H), 1.84-1.62 (m, 4H), 1.46 (s, 9H).

Step 8: tert-butyl 3-[7-[6-[bis[(4-methoxyphenyl)methyl]amino]-4-methyl-3-(trifluoromethyl)-2-pyridyl]-6-chloro-2,8-difluoro-quinazolin-4-yl]-3,8-diazabicyclo[3.2.1]octane-8-carboxylate (Intermediate 1)

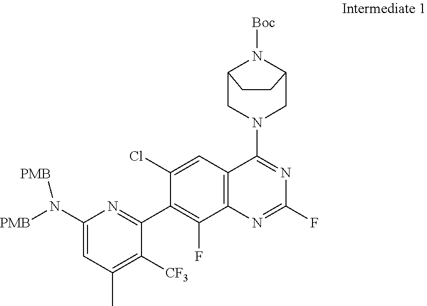

To a solution of tert-butyl 3-[7-[6-[bis[(4-methoxyphenyl)methyl]amino]-3-iodo-4-methyl-2-pyridyl]-6-chloro-2,8-difluoro-quinazolin-4-yl]-3,8-diazabicyclo[3.2.1]octane-8-carboxylate (510 mg, 0.57 mmol, 1 eq) in N,N-dimethylacetamide (10 mL) was added cuprous iodide (1.32 g, 6.93 mmol, 12 eq) and methyl 2,2-difluoro-2-fluorosulfonyl-acetate (2.77 g, 14.44 mmol, 1.8 mL, 25 eq). The mixture was stirred at 90° C. for 12 hours. LCMS showed the starting material was consumed completely and one main peak with desired mass was detected. The mixture was diluted with ethyl acetate (20 mL) and filtered. To the filtrate was added water (20 mL) before the mixture was extracted by ethyl acetate (20 mL×3). The combined organic layers were washed with brine (20 mL×3), dried over anhydrous sodium sulfate, filtered and concentrated under reduced pressure to give a residue. The residue was purified by prep-TLC (silicon dioxide, petroleum ether/ethyl acetate=2/1) to give the desired product (300 mg, 0.33 mmol, 57% yield, 92% purity) as a white solid. LCMS (ESI, m/z): 825.5[M]⁺. ¹H NMR: (400 MHz, DMSO-d₆) δ: 8.07 (s, 1H), 7.16 (d, J=8.8 Hz, 4H), 6.94-6.79 (m, 5H), 4.87-4.68 (m, 2H), 4.62-4.46 (m, 3H), 4.38-4.20 (m, 3H), 3.72 (s, 6H), 3.61-3.51 (m, 1H), 2.40 (s, 3H), 1.85-1.63 (m, 4H), 1.46 (s, 9H).

Synthesis of Intermediate 2: tert-butyl 3-[3-[(2S)-2-(hydroxymethyl)pyrrolidin-1-yl]propoxy]propanoate

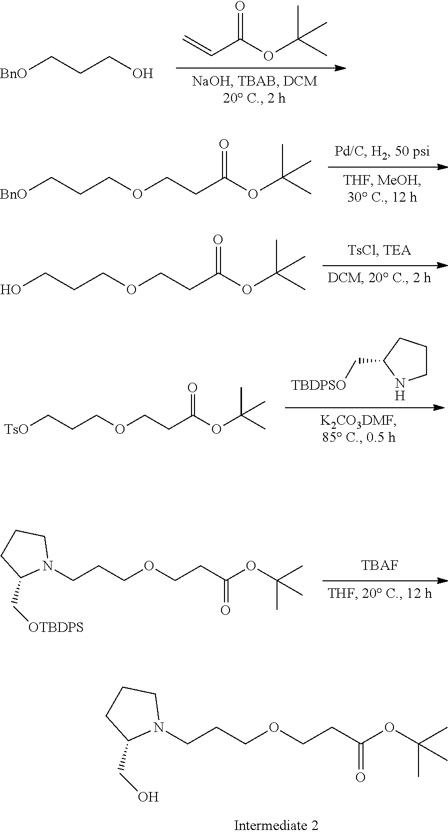

Step 1: tert-butyl 3-(3-benzyloxypropoxy)propanoate

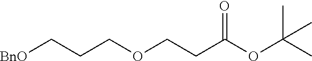 [SMILES: CC(C)(C)OC(=O)CCOCCCOCc1ccccc1]

To a solution of 3-benzyloxypropan-1-ol (10 g, 60.16 mmol, 1 eq) and tert-butyl but-3-enoate (8.55 g, 60.16 mmol, 1 eq) in dichloromethane (100 mL) was added aqueous sodium hydroxide (14.44 g, 180.49 mmol, 50% w/w, 3 eq) and tetrabutylammonium bromide (3.88 g, 12.03 mmol, 0.2 eq). Then the mixture was stirred at 20° C. for 2 hours. TLC (Petroleum ether/Ethyl acetate=10/1) showed 3-benzyloxypropan-1-ol was consumed completely and a new spot was detected. The mixture was diluted with water (200 mL), extracted with dichloromethane (200 mL×3). The combined organic layers were washed with brine (200 mL), dried over sodium sulfate, filtered and then concentrated under vacuum to get a residue which was purified by column chromatography (silicon dioxide, petroleum ether/ethyl acetate=100/1 to 10/1) to afford the desired the product (16.5 g, 56.05 mmol, 93% yield) as a colorless oil. ¹H NMR: (400 MHz, CDCl₃) δ: 7.33-7.16 (m, 5H), 4.42 (s, 2H), 3.58 (s, 2H), 3.52-3.43 (m, 4H), 2.45-2.34 (m, 2H), 1.80 (t, J=6.4 Hz, 2H), 1.38 (s, 9H).

Step 2: tert-butyl 3-(3-hydroxypropoxy)propanoate

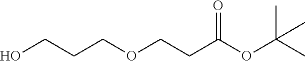

To a solution of tert-butyl 3-(3-benzyloxypropoxy)propanoate (16.5 g, 56.05 mmol, 1 eq) in tetrahydrofuran (100 mL) and methanol (100 mL) was added palladium on carbon (2 g, 10% purity). Then the mixture was stirred at 30° C. under hydrogen (50 psi) for 12 hours. TLC (Petroleum ether/Ethyl acetate=5/1) showed tert-butyl 3-(3-benzyloxypropoxy)propanoate was consumed completely and a new spot was detected. The mixture was filtered and then concentrated under vacuum to get the crude product tert-butyl 3-(3-hydroxypropoxy)propanoate (11.2 g, 54.83 mmol, 97% yield), which was used into the next step directly.

Step 3: tert-butyl 3-[3-(p-tolylsulfonyloxy)propoxy]propanoate

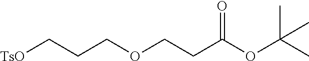 [SMILES: Cc1ccc(S(=O)(=O)OCCCOCCC(=O)OC(C)(C)C)cc1]

To a solution of tert-butyl 3-(3-hydroxypropoxy)propanoate (11 g, 53.85 mmol, 1 eq) in dichloromethane (110 mL) was added p-toluensulfonyl chloride (15.40 g, 80.78 mmol, 1.5 eq) and triethylamine (16.35 g, 161.56 mmol, 3 eq). Then the mixture was stirred at 20° C. for 2 hours. TLC (petroleum ether/ethyl acetate=3/1) showed tert-butyl 3-(3-hydroxypropoxy) propanoate was consumed completely and a new spot was detected. The mixture was diluted with water (100 mL), extracted with dichloromethane (100 mL×2), washed with brine (100 mL). The combined organic layers were dried over sodium sulfate, filtered and then concentrated under vacuum to get a residue. The residue was purified by silica gel column chromatography (silicon dioxide, Petroleum ether/Ethyl acetate=100/1 to 8/1) to afford the desired product (16 g, 44.64 mmol, 83% yield) as a colorless oil. ¹H NMR: (400 MHz, CDCl₃) δ: 7.85-7.76 (m, 2H), 7.36 (d, J=8.0 Hz, 2H), 4.13 (t, J=6.4 Hz, 2H), 3.58 (t, J=6.4 Hz, 2H), 3.46 (t, J=6.0 Hz, 2H), 2.47 (s, 3H), 2.41 (t, J=6.4 Hz, 2H), 1.95-1.86 (m, 2H), 1.45 (s, 9H).

Step 4: tert-butyl-diphenyl-[[(2S)-pyrrolidin-2-yl] methoxy]silane

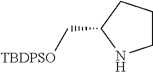

To a solution of [(2S)-pyrrolidin-2-yl]methanol (2 g, 19.77 mmol, 1 eq) and tert-butyldimethylsilyl chloride (6.52 g, 23.73 mmol, 1.2 eq) in tetrahydrofuran (20 mL) was added triethylamine (6.00 g, 59.32 mmol, 3 eq) and dimethylaminopyridine (241 mg, 1.98 mmol, 0.1 eq). Then the mixture was stirred at 20° C. for 1 hour. LCMS showed the desired mass was detected. The mixture was concentrated under vacuum to get a residue. The residue was purified by column chromatography (silicon dioxide, Dichloromethane/Methanol=1/0 to 10/1) to give the desired product (4.5 g, 13.25 mmol, 67% yield) as a yellow solid. LCMS (ESI, m/z): 340.4[M+1]⁺.

Step 5: tert-butyl 3-[3-[(2S)-2-[[tert-butyl(diphenyl)silyl]oxymethyl]pyrrolidin-1-yl]propoxy]propanoate

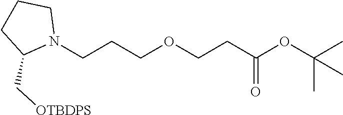

To a solution of tert-butyl-diphenyl-[[(2S)-pyrrolidin-2-yl]methoxy]silane (1 g, 2.95 mmol, 1 eq) and tert-butyl 3-[3-(p-tolylsulfonyloxy)propoxy]propanoate (1.06 g, 2.95 mmol, 1 eq) in N,N-dimethylformamide (10 mL) was added potassium carbonate (1.22 g, 8.84 mmol, 3 eq). Then the mixture was stirred at 85° C. for 0.5 hour. TLC (petroleum ether/ethyl acetate=1/1) showed tert-butyl-diphenyl-[[(2S)-pyrrolidin-2-yl]methoxy]silane was consumed completely and a new spot was detected. The mixture was diluted with water (50 mL), extracted with ethyl acetate (50 mL×3). The combined organic layers were washed with brine (50 mL), dried over sodium sulfate, filtered and then concentrated under vacuum to get a residue which was purified by column chromatography (silicon dioxide, petroleum ether/ethyl acetate=10/1 to 1/1) to give the desired product (950 mg, 1.81 mmol, 61% yield) as a yellow oil. ¹H NMR: (400 MHz, CDCl₃) δ: 7.66-7.56 (m, 4H), 7.40-7.28 (m, 6H), 3.65-3.51 (m, 3H), 3.46-3.29 (m, 3H), 3.07-2.98 (m, 1H), 2.83-2.70 (m, 1H), 2.53 (br s, 1H), 2.39 (t, J=6.8 Hz, 2H), 2.30-2.20 (m, 1H), 2.17-2.07 (m, 1H), 1.89-1.76 (m, 1H), 1.63 (d, J=4.4 Hz, 5H), 1.39 (s, 9H), 1.00 (s, 9H).

Step 6: tert-butyl 3-[3-[(2S)-2-(hydroxymethyl)pyrrolidin-1-yl]propoxy]propanoate (Intermediate 2)

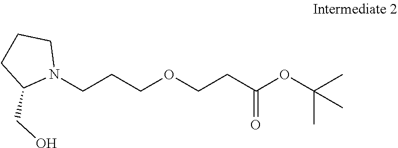 [SMILES: CC(C)(C)OC(=O)CCOCCCN1CCC[C@H]1CO]

To a solution of tert-butyl 3-[3-[(2S)-2-[[tert-butyl(diphenyl)silyl]oxymethyl]pyrrolidin-1-yl] propoxy]propanoate (900 mg, 1.71 mmol, 1 eq) in tetrahydrofuran (10 mL) was added tetrabutylammonium fluoride (0.5 M, 4.1 mL, 1.2 eq) at 0° C., then the mixture was stirred at 20° C. for 12 hours. TLC (dichloromethane/methanol=10/1) showed tert-butyl 3-[3-[(2S)-2-[[tert-butyl(diphenyl)silyl]oxymethyl]pyrrolidin-1-yl]propoxy]propanoate was consumed completely and a new spot was detected. The mixture was concentrated under vacuum to get a residue which was purified by prep-TLC (silicon dioxide, dichloromethane/methanol =10/1) to generate the desired product (400 mg, 1.39 mmol, 81% yield) as a yellow oil.

¹H NMR: (400 MHz, CDCl₃) δ: 3.63-3.52 (m, 3H), 3.50-3.36 (m, 3H), 3.35-3.29 (m, 1H), 3.16-3.07 (m, 1H), 2.85-2.74 (m, 1H), 2.54 (d, J=3.2 Hz, 1H), 2.41 (t, J=6.4 Hz, 2H), 2.31-2.15 (m, 2H), 1.84-1.63 (m, 6H), 1.44-1.29 (m, 9H).

Synthesis of Intermediate 3: [(2S)-1-(7-tetrahydropyran-2-yloxyheptyl)pyrrolidin-2-yl]methanol

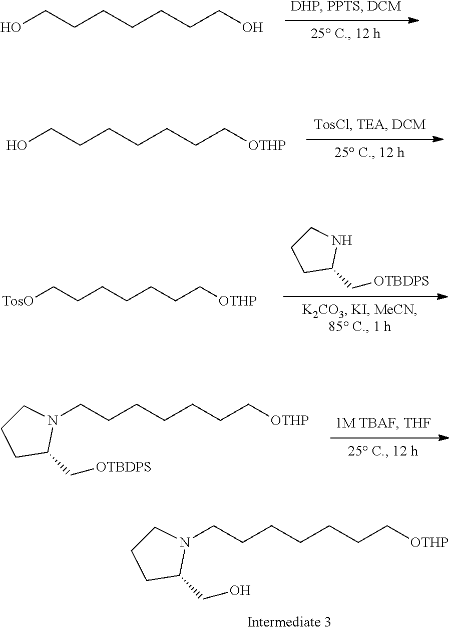

Step 1: 7-tetrahydropyran-2-yloxyheptan-1-ol

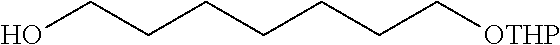

A solution of heptane-1,7-diol (13 g, 98.34 mmol, 1 eq) and pyridinium p-toluenesulfonate (1.24 g, 4.92 mmol, 0.05 eq) in dichloromethane (200 mL) was added 3,4-dihydro-pyran (8.27 g, 98.34 mmol, 9.0 mL, 1 eq) at 0° C., the mixture was stirred for 12 hours at 25° C. TLC showed the reaction completed. The reaction mixture was quenched with water (200 mL) and then extracted with dichloromethane (200 mL×3). The combined organic layers were washed with brine (200 mL), dried with anhydrous sodium sulfate, filtered and concentrated in vacuum to give a residue which was purified by silica gel column chromatography (petroleum ether/ethyl acetate=20/1 to 10/1) to give the desired product (11 g, 50.85 mmol, 52% yield) as a yellow oil. ¹H NMR: (400 MHz, CDCl₃) δ: 4.61-4.51 (m, 1H), 3.87-3.81 (m, 1H), 3.76-3.73 (m, 1H), 3.63-3.60 (m, 2H), 3.52-3.45 (m, 1H), 3.42-3.31 (m, 1H), 1.92-1.80 (m, 1H), 1.77-1.65 (m, 1H), 1.61-1.50 (m, 9H), 1.42-1.30 (m, 6H).

Step 2: 7-tetrahydropyran-2-yloxyheptyl 4-methylbenzenesulfonate

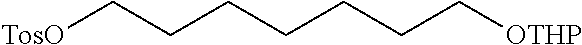

To a solution of 7-tetrahydropyran-2-yloxyheptan-1-ol (10.8 g, 49.93 mmol, 1 eq) and triethylamine (15.16 g, 149.78 mmol, 20.8 mL, 3eq) in dichloromethane (200 mL) was added p-toluenesulfonyl chloride (11.42 g, 59.91 mmol, 1.2 eq) at 0° C., and the mixture was stirred for 12 hours at 25° C. TLC showed the reaction was completed. The reaction mixture was quenched by water (200 mL) and then extracted with dichloromethane (200 mL×3). The combined organic layers were washed with brine (200 mL), dried with anhydrous sodium sulfate, filtered and concentrated in vacuum to give a residue which was purified by silica gel chromatography (petroleum ether/ethyl acetate=100/1 to 1/1) to generate the desired product (9 g, 24.29 mmol, 49% yield) as a yellow oil.

Step 3: tert-butyl-diphenyl-[[(2S)-1-(7-tetrahydropyran-2-yloxyheptyl)pyrrolidin-2-yl]methoxy]silane

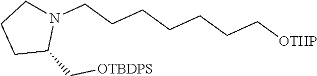

To a solution of 7-tetrahydropyran-2-yloxyheptyl 4-methylbenzenesulfonate (1.6 g, 4.32 mmol, 1 eq) and tert-butyl-diphenyl-[[(2S)-pyrrolidin-2-yl]methoxy]silane (1.76 g, 5.18 mmol, 1.2 eq) in N,N-dimethylformamide (16 mL), was added potassium carbonate (1.19 g, 8.64 mmol, 2 eq) and potassium iodide (71 mg, 0.43 mmol, 0.1 eq). The mixture was stirred at 85° C. for 1 hour. LCMS showed that the desired mass was detected. Ethyl acetate (40 mL) and water (60 mL) were added before the mixture was extracted with ethyl acetate (40 mL×2). The combined organic layers were washed with brine (30 mL×3), dried over anhydrous sodium sulfate, filtered and concentrated under reduced pressure to give a residue which was purified by silica gel column chromatography (petroleum ether/ethyl acetate=1/1) to afford the desired product (1.6 g, 2.97 mmol, 68% yield) as a yellow oil. LCMS: (ESI, m/z): 538.3[M]⁺.

Step 4: [(2S)-1-(7-tetrahydropyran-2-yloxyheptyl)pyrrolidin-2-yl]methanol (Intermediate 3)

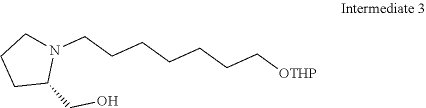

To a solution of tert-butyl-diphenyl-[[(2S)-1-(7-tetrahydropyran-2-yloxyheptyl)pyrrolidin-2-yl]methoxy]silane (700 mg, 1.30 mmol, 1 eq) in tetrahydrofuran (7 mL) was added tetrabutylammonium fluoride solution (1 M, 2.6 mL, 2 eq). The mixture was stirred at 25° C. for 12 hours. TLC (dichloromethane/methanol=10/1 with 1% ammonium hydroxide) showed that the reaction was completed. The reaction mixture was concentrated under reduce pressure to give a residue which was purified by silica gel column chromatography (petroleum ether/ethyl acetate=1/1 with 1% ammonium hydroxide) to afford the desired product (300 mg, 1.00 mmol, 77% yield) as a yellow oil.

¹H NMR: (400 MHz, CDCl₃) δ: 4.65-4.51 (m, 1H), 3.87-3.84 (m, 1H), 3.76-3.73 (m, 1H), 3.63 (dd, J=3.6, 10.8 Hz, 1H), 3.56-3.45 (m, 1H), 3.44-3.35 (m, 2H), 3.22-3.14 (m, 1H), 2.71-2.65 (m, 1H), 2.62-2.57 (m, 1H), 2.29-2.19 (m, 2H), 1.92-1.67 (m, 6H), 1.64-1.46 (m, 8H), 1.41-1.23 (m, 6H).

Example 1: (2S,4R)-1-[(2S)-2-[3-[3-[(2S)-2-[[7-[6-amino-4-methyl-3-(trifluoromethyl)-2-pyridyl]-6-chloro-4-(3,8-diazabicyclo[3.2.1]octan-3-yl)-8-fluoro-quinazolin-2-yl]oxymethyl]pyrrolidin-1-yl]propoxy]propanoylamino]-3,3-dimethyl-butanoyl]-4-hydroxy-N-[(1S)-1-[4-(4-methylthiazol-5-yl)phenyl]ethyl]pyrrolidine-2-carboxamide

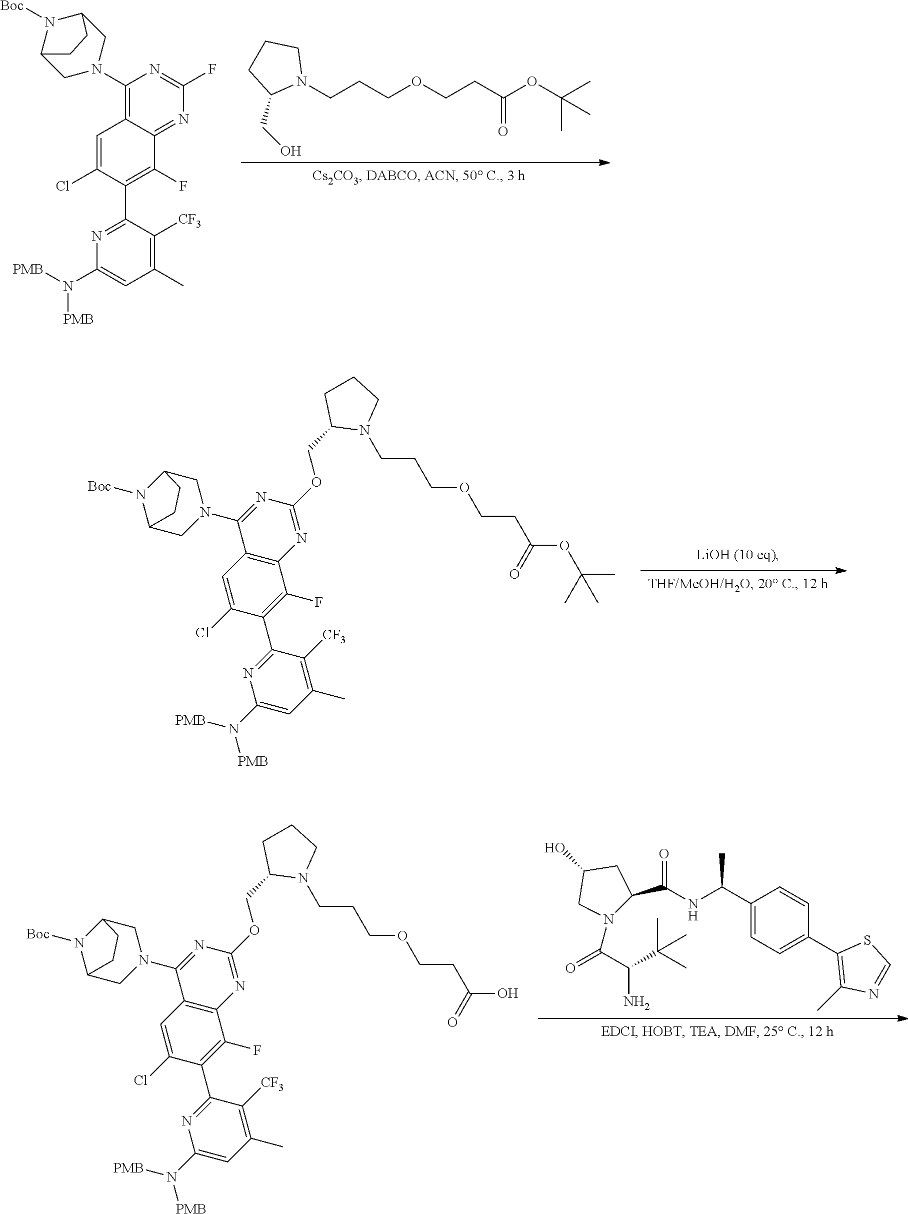

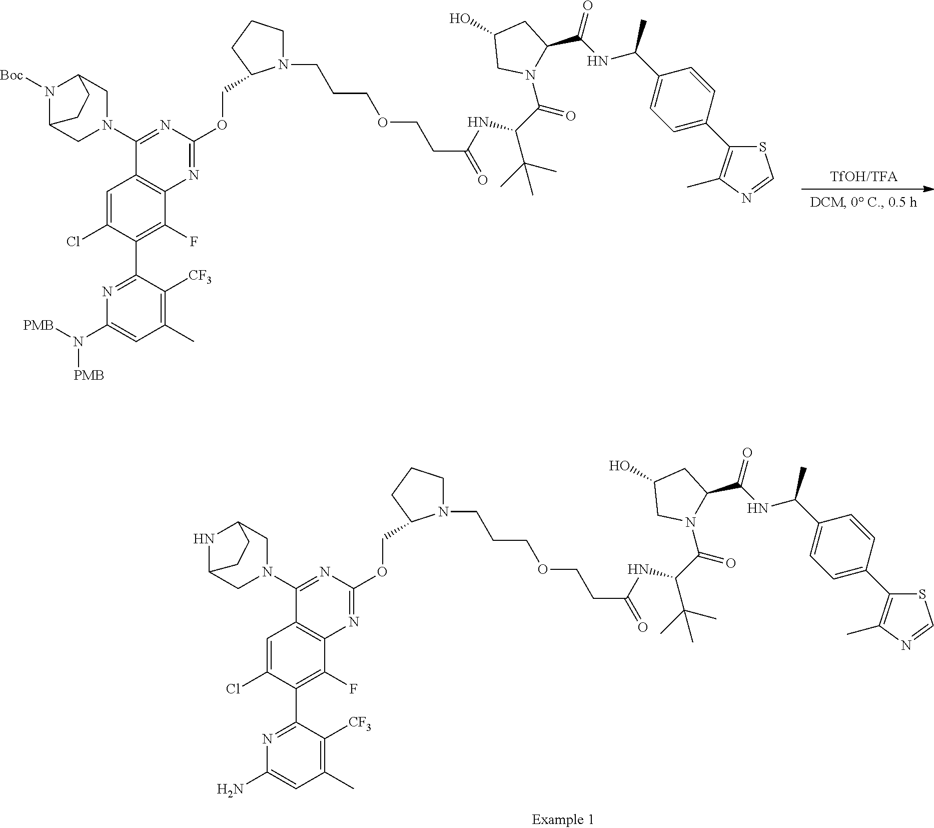

Step 1: tert-butyl 3-[7-[6-[bis[(4-methoxyphenyl)methyl] amino]-4-methyl-3-(trifluoromethyl)-2-pyridyl]-2-[[(2S)-1-[3-(3-tert-butoxy-3-oxo-propoxy)propyl]pyrrolidin-2-yl]methoxy]-6-chloro-8-fluoro-quinazolin-4-yl]-3,8-diazabicyclo [3.2.1]octane-8-carboxylate

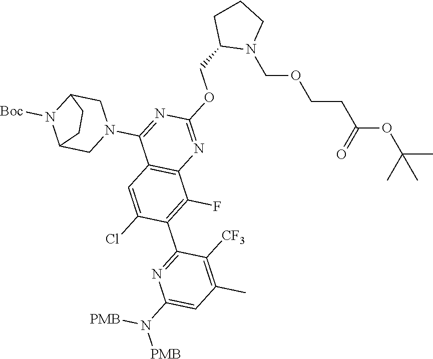

To a solution of tert-butyl 3-[7-[6-[bis[(4-methoxyphenyl)methyl]amino]-4-methyl-3-(trifluoromethyl)-2-pyridyl]-6-chloro-2,8-difluoro-quinazolin-4-yl]-3,8-diazabicyclo[3.2.1]octane-8-carboxylate (1 g, 1.21 mmol, 1 eq) and tert-butyl 3-[3-[(2S)-2-(hydroxymethyl) pyrrolidin-1-yl]propoxy]propanoate (522 mg, 1.82 mmol, 1.5 eq) in acetonitrile (1 mL) was added cesium carbonate (789 mg, 2.42 mmol, 2 eq) and 1,4-diazabicyclo[2.2.2]octane (13 mg, 0.12 mmol, 0.1 eq). Then the reaction mixture was stirred at 50° C. for 3 hours. LCMS showed the desired mass was detected. The mixture was filtered and then concentrated under reduced pressure to get a residue which was purified by prep-TLC (silicon dioxide, dichloromethane\methanol=201) to afford the product (700 mg, 0.64 mmol, 53% yield) as a yellow solid. LCMS: (ESI, m/z): 1092.8 [M]+. ¹H NMR: (400 MHz, CDCl₃) δ: 7.68 (s, 1H), 7.17 (d, J=8.8 Hz, 4H), 6.87 (d, J=8.8 Hz, 4H), 6.43 (s, 1H), 4.86-4.74 (m, 2H), 4.58-4.17 (m, 10H), 3.84-3.80 (m, 6H), 3.65 (t, J=6.4 Hz, 3H), 3.51 (br s, 1H), 3.20-2.89 (m, 4H), 2.52-2.39 (m, 7H), 2.02-1.78 (m, 10H), 1.54 (s, 8H), 1.44 (s, 9H).

Step 2: 3-[3-[(2S)-2-[[7-[6-[bis[(4-methoxyphenyl) methyl]amino]-4-methyl-3-(trifluoromethyl)-2-pyridyl]-4-(8-tert-butoxycarbonyl-3,8-diazabicyclo[3.2.1]octan-3-yl)-6-chloro-8-fluoro-quinazolin-2-yl]oxymethyl]pyrrolidin-1-yl]propoxy]propanoic acid

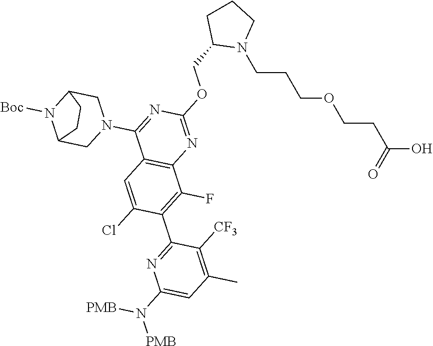

To a solution of tert-butyl 3-[7-[6-[bis[(4-methoxyphenyl)methyl]amino]-4-methyl-3-(trifluoromethyl)-2-pyridyl]-2-[[(2S)-1-[3-(3-tert-butoxy-3-oxo-propoxy)propyl]pyrrolidin-2-yl]methoxy]-6-chloro-8-fluoro-quinazolin-4-yl]-3,8-diazabicyclo[3.2.1]octane-8-carboxylate (700 mg, 0.64 mmol, 1 eq) in methanol (2 mL), tetrahydrofuran (2 mL) and water (2 mL) was added lithium hydroxide (269 mg, 6.41 mmol, 10 eq). Then the reaction mixture was stirred at 20° C. for 12 hours. LCMS showed the desired mass was detected. The mixture was concentrated under reduced pressure to remove tetrahydrofuran and methanol, then adjusted pH with aqueous hydrochloric (1M, 0.8 mL) to about 5. The mixture was extracted with ethyl acetate (30 mL×2), washed with brine (30 mL), dried over sodium sulfate, filtered and then concentrated under vacuum to get the crude product (600 mg, 50.59 mmol, 90% yield) as a yellow solid and the crude product was used into the next step directly. LCMS: (ESI, m/z): 1036.6 [M]⁺.

Step 3: tert-butyl 3-[7-[6-[bis[(4-methoxyphenyl)methyl] amino]-4-methyl-3-(trifluoromethyl)-2-pyridyl]-6-chloro-8-fluoro-2-[[(2S)-1-[3-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]propyl]pyrrolidin-2-yl]methoxy]quinazolin-4-yl]-3,8-diazabicyclo[3.2.1]octane-8-carboxylate

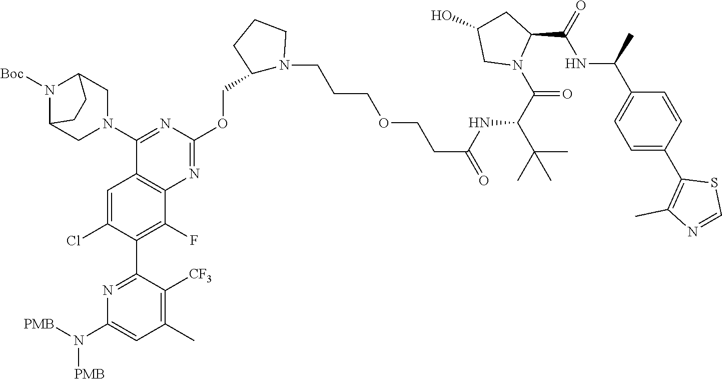

To a solution of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (69 mg, 0.14 mmol, 1.5 eq, hydrochloride) and 3-[3-[(2S)-2-[[7-[6-[bis[(4-methoxyphenyl)methyl]amino]-4-methyl-3-(trifluoromethyl)-2-pyridyl]-4-(8-tert-butoxycarbonyl-3,8-diazabicyclo[3.2.1]octan-3-yl)-6-chloro-8-fluoro-quinazolin-2-yl]oxymethyl]pyrrolidin-1-yl]propoxy]propanoic acid (100 mg, 0.096 mmol, 1 eq) in N,N-dimethylformamide (2 mL) was added N,N-diisopropylethylamine (62 mg, 0.48 mmol, 5 eq) and 1-hydroxybenzotriazole (26 mg, 0.19 mmol, 2 eq). Then the reaction mixture was stirred at 20° C. for 0.5 hour. Then1-(3-dimethylaminopropyl)-3-ethylcarbodiimide hydrochloride (46 mg, 0.24 mmol, 2.5 eq) was added into the mixture and stirred at 20° C. for 11.5 hours. LCMS showed the desired mass was detected. The mixture was diluted with water (10 mL), extracted with ethyl acetate (20 mL×2). The combined organic layers were washed with brine (20 mL), dried over sodium sulfate, filtered and then concentrated under reduced pressure to get a residue which was purified by prep-TLC (silicon dioxide, dichloromethane/methanol=10/1) to give the desired product (100 mg, 0.069 mmol, 71% yield) as a yellow oil. LCMS: (ESI, m/z): 1463.1 [M]⁺.

Step 4: (2S,4R)-1-[(2S)-2-[3-[3-[(2S)-2-[[7-[6-amino-4-methyl-3-(trifluoromethyl)-2-pyridyl]-6-chloro-4-(3,8-diazabicyclo[3.2.1]octan-3-yl)-8-fluoro-quinazolin-2-yl]oxymethyl]pyrrolidin-1-yl]propoxy]propanoylamino]-3,3-dimethyl-butanoyl]-4-hydroxy-N-[(1S)-1-[4-(4-methylthiazol-5-yl)phenyl]ethyl]pyrrolidine-2-carboxamide (Example 1)

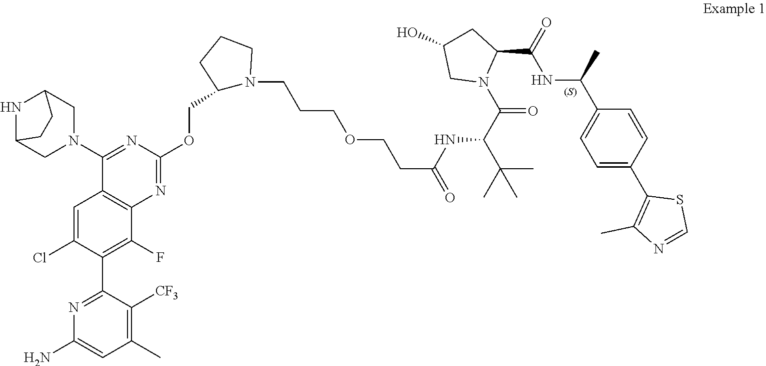

To a solution of tert-butyl 3-[7-[6-[bis[(4-methoxyphenyl)methyl]amino]-4-methyl-3-(trifluoromethyl)-2-pyridyl]-6-chloro-8-fluoro-2-[[(2S)-1-[3-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]propyl]pyrrolidin-2-yl]methoxy]quinazolin-4-yl]-3,8-diazabicyclo[3.2.1]octane-8-carboxylate (100 mg, 0.07 mmol, 1 eq) in dichloromethane (2 mL) was added trifluoroacetic acid (0.5 mL) at 0° C. Then trifluoromethanesulfonic acid (0.025 mL) was added into the mixture at 0° C. and the reaction solution was stirred at 0° C. for 0.5 hour. LCMS showed the desired mass was detected. The mixture was concentrated under reduced pressure to get a residue which was purified by prep-HPLC (column: Phenomenex Synergi C18 150*25 mm*10 um; mobile phase: [water(0.225% FA)-ACN]; B %: 13%-33%, 10 min) to get the product (16.71 mg, 0.014 mmol, 21% yield, 98% purity) as a white solid. LCMS: (ESI, m/z): 1122.4 [M]⁺. ¹H NMR: (400 MHz, DMSO-d₆) δ: 8.98 (s, 1H), 8.37 (d, J=8.0 Hz, 1H), 8.23 (s, 2H), 7.86-7.76 (m, 2H), 7.46-7.31 (m, 4H), 6.84 (br s, 2H), 6.49 (s, 1H), 4.95-4.86 (m, 1H), 4.51 (d, J=9.6 Hz, 1H), 4.41 (t, J=8.0 Hz, 1H), 4.37-4.18 (m, 5H), 4.11-3.99 (m, 2H), 3.42-3.29 (m, 8H), 3.06-3.02 (m, 1H), 2.93-2.73 (m, 3H), 2.57-2.52 (m, 1H), 2.45 (s, 3H), 2.40-2.33 (m, 4H), 2.31-2.23 (m, 1H), 2.21-2.14 (m, 1H), 2.05-1.96 (m, 1H), 1.94-1.85 (m, 1H), 1.83-1.75 (m, 1H), 1.73-1.59 (m, 9H), 1.36 (d, J=7.2 Hz, 3H), 0.89 (s, 9H).

Example 2: (2S,4R)-1-[(2S)-2-[3-[3-[(2S)-2-[[7-[6-amino-4-methyl-3-(trifluoromethyl)-2-pyridyl]-6-chloro-4-(3,8-diazabicyclo[3.2.1]octan-3-yl)-8-fluoro-quinazolin-2-yl]oxymethyl]pyrrolidin-1-yl]propoxy]propanoylamino]-3,3-dimethyl-butanoyl]-4-hydroxy-N-[[4-(4-methylthiazol-5-yl)phenyl]methyl]pyrrolidine-2-carboxamide

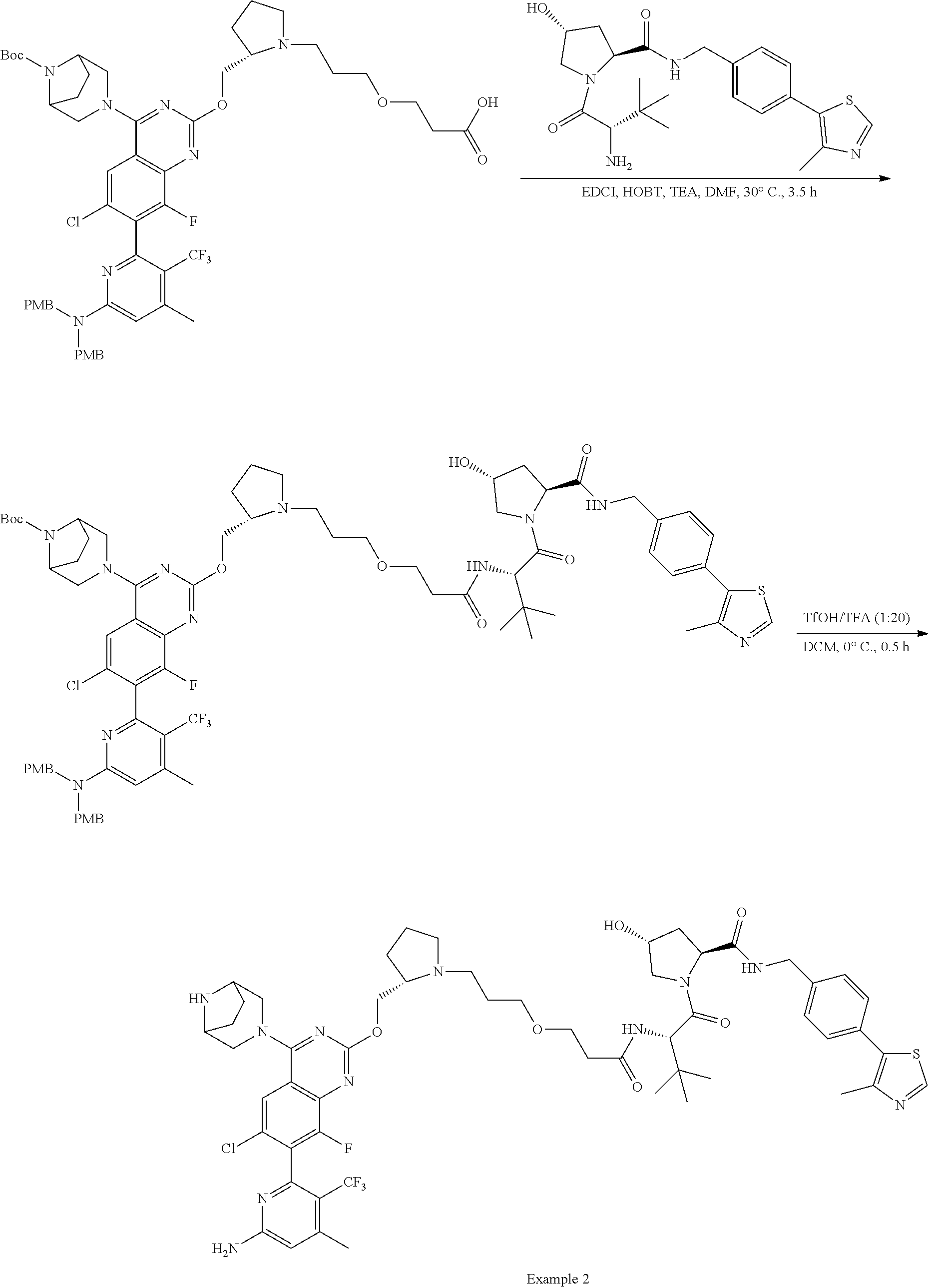

Step 1: tert-butyl 3-[7-[6-[bis[(4-methoxyphenyl)methyl]amino]-4-methyl-3-(trifluoromethyl)-2-pyridyl]-6-chloro-8-fluoro-2-[[(2S)-1-[3-[3-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-3-oxo-propoxy]propyl]pyrrolidin-2-yl]methoxy]quinazolin-4-yl]-3,8-diazabicyclo[3.2.1]octane-8-carboxylate

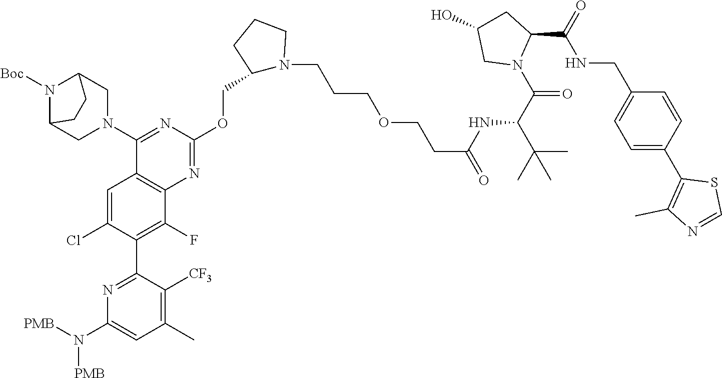

To a solution of 3-[3-[(2S)-2-[[7-[6-[bis[(4-methoxyphenyl)methyl]amino]-4-methyl-3-(trifluoromethyl)-2-pyridyl]-4-(8-tertbutoxycarbonyl-3,8-diazabicyclo[3.2.1]octan-3-yl)-6-chloro-8-fluoro-quinazolin-2-yl]oxymethyl]pyrrolidin-1-yl]propoxy]propanoic acid (100 mg, 0.096 mmol, 1 eq) and (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (93 mg, 0.22 mmol, 2.23 eq) in N,N-dimethylformamide (2 mL) was added N,N-diisopropylethylamine (62 mg, 0.48 mmol, 5 eq) and 1-hydroxybenzotriazole (26 mg, 0.19 mmol, 2 eq). The mixture was stirred at 30° C. for 0.5 hour. Then N-(3-dimethylaminopropyl)-N-ethylcarbodiimide hydrochloride (37 mg, 0.19 mmol, 2 eq) was added. The mixture was stirred at 30° C. for 3 hours. LCMS showed the desired mass was detected. Dichloromethane (15 mL) and water (15 mL) were added before the mixture was extracted with dichloromethane (15 mL×2). The combined organic layers were washed with brine (15 mL), dried over anhydrous sodium sulfate, filtered and concentrated under reduced pressure to give a residue which was purified by prep-TLC (silicon dioxide, dichloromethane: methanol=10:1) to afford the desired product (110 mg, 0.076 mmol, 73% yield) as a white solid. LCMS: (ESI, m/z): 1448.3 [M]⁺.

Step 2: (2S,4R)-1-[(2S)-2-[3-[3-[(2S)-2-[[7-[6-amino-4-methyl-3-(trifluoromethyl)-2-pyridyl]-6-chloro-4-(3,8-diazabicyclo[3.2.1]octan-3-yl)-8-fluoro-quinazolin-2-yl]oxymethyl]pyrrolidin-1-yl]propoxy]propanoylamino]-3,3-dimethyl-butanoyl]-4-hydroxy-N-[[4-(4-methylthiazol-5-yl)phenyl]methyl]pyrrolidine-2-carboxamide (Example 2)

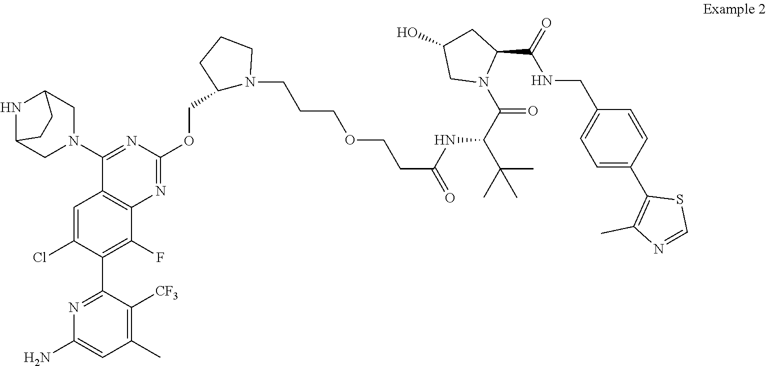 [SMILES: Cc1cc(N)nc(-c2c(Cl)cc3c(N4CC5CCC(C4)N5)nc(OC[C@@H]4CCCN4CCCOCCC(=O)N[C@H](C(=O)N4C[C@H](O)C[C@H]4C(=O)NCc4ccc(-c5scnc5C)cc4)C(C)(C)C)nc3c2F)c1C(F)(F)F]

To a solution of tert-butyl 3-[7-[6-[bis[(4-methoxyphenyl)methyl]amino]-4-methyl-3-(trifluoromethyl)-2-pyridyl]-6-chloro-8-fluoro-2-[[(2S)-1-[3-[3-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-3-oxo-propoxy]propyl]pyrrolidin-2-yl]methoxy]quinazolin-4-yl]-3,8-diazabicyclo[3.2.1]octane-8-carboxylate (110 mg, 0.076 mmol, 1 eq) in dichloromethane (1 mL) was added trifluoroacetic acid (0.5 mL). Then trifluoromethanesulfonic acid (0.025 mL) was added into the mixture at 0° C. and the reaction solution was stirred at 0° C. for 0.5 hour. LCMS showed the desired mass was detected. The reaction mixture was quenched by water (1 mL) at 0° C., and then extracted with dichloromethane (5 mL×3). The combined organic layers were dried over anhydrous sodium sulfate, filtered and concentrated under reduced pressure to give a residue which was purified by prep-HPLC (column: Phenomenex Synergi C18 150*25 mm*10 um; mobile phase: [water (0.225% formic acid)-acetonitrile]; B %:13%-33%, 10 min) to afford the desired product (14.91 mg, 0.013 mmol, 17% yield, 98% purity, formate) as a white solid. LCMS: (ESI, m/z): 1108.4 [M]⁺. ¹H NMR: (400 MHz, DMSO-d₆) δ: 8.97 (s, 1H), 8.56 (t, J=6.4 Hz, 1H), 8.23 (s, 2H), 7.91-7.82 (m, 1H), 7.80 (s, 1H), 7.46-7.33 (m, 4H), 6.83 (m, 2H), 6.48 (s, 1H), 4.53 (d, J=8.4 Hz, 1H), 4.47-4.38 (m, 2H), 4.36-4.27 (m, 3H), 4.25-4.18 (m, 2H), 4.07-3.99 (m, 1H), 3.62 (m, 4H), 3.56 (d, J=5.6 Hz, 6H), 3.05-2.99 (m, 2H), 2.92-2.81 (m, 2H), 2.80-2.74 (m, 1H), 2.44-2.41 (m, 3H), 2.38-2.23 (m, 6H), 2.19 (s, 1H), 2.05-1.99 (m, 1H), 1.93-1.84 (m, 2H), 1.72-1.59 (m, 9H), 0.89 (s, 9H).

Example 3: (2S,4R)—N-[[2-[3-[3-[(2S)-2-[[7-[6-amino-4-methyl-3-(trifluoromethyl)-2-pyridyl]-6-chloro-4-(3,8-diazabicyclo[3.2.1]octan-3-yl)-8-fluoro-quinazolin-2-yl]oxymethyl]pyrrolidin-1-yl]propoxy]propoxy]-4-(4-methylthiazol-5-yl)phenyl]methyl]-1-[(2R)-2-[(1-fluorocyclopropanecarbonyl)amino]-3,3-dimethyl-butanoyl]-4-hydroxy-pyrrolidine-2-carboxamide

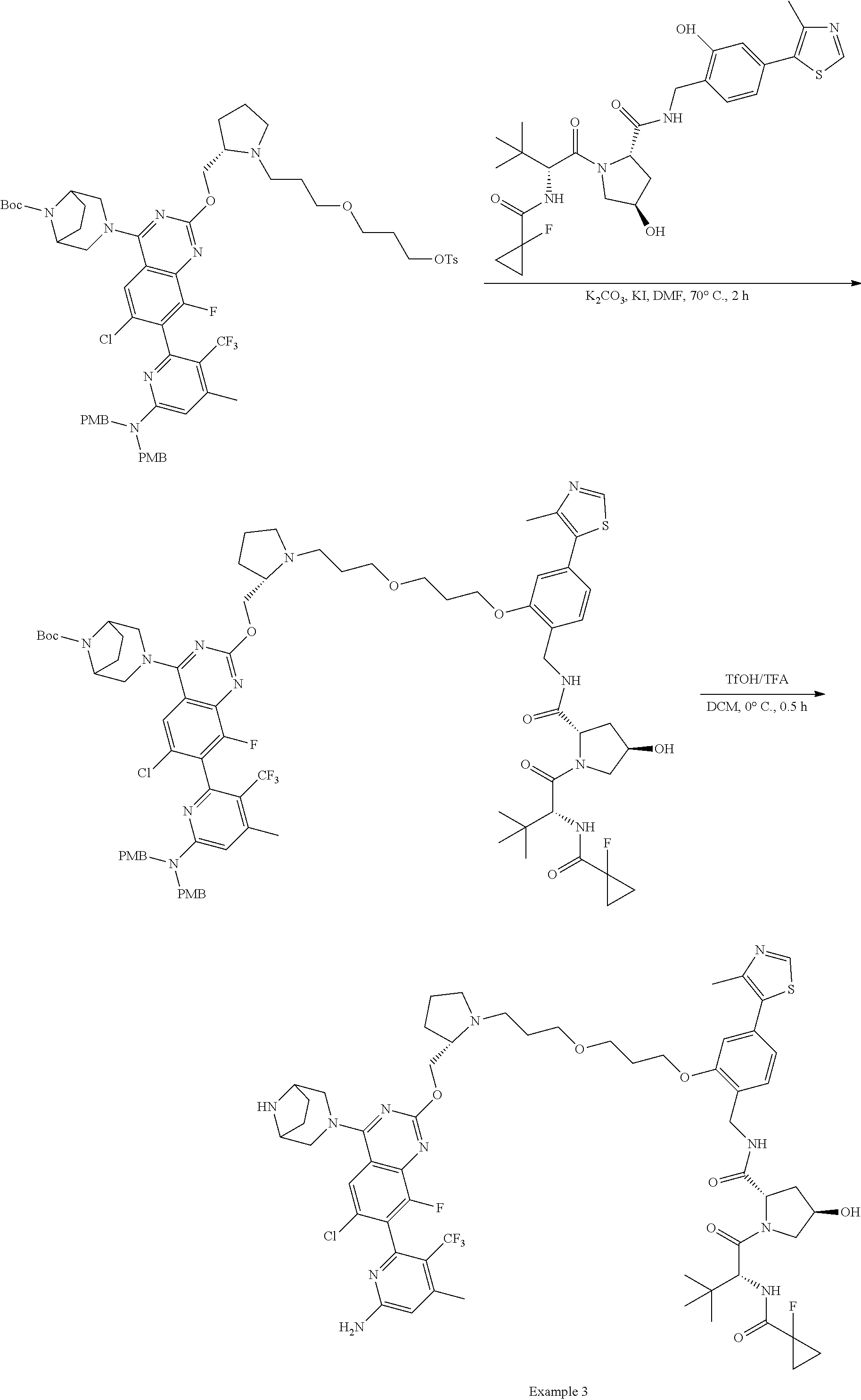

Step 1: tert-butyl 3-[7-[6-[bis[(4-methoxyphenyl)methyl]amino]-4-methyl-3-(trifluoromethyl)-2-pyridyl]-6-chloro-8-fluoro-2-[[(2S)-1-[3-[3-[2-[[[(2S,4R)-1-[(2R)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]propoxy]propyl]pyrrolidin-2-yl]methoxy]quinazolin-4-yl]-3,8-diazabicyclo[3.2.1]octane-8-carboxylate

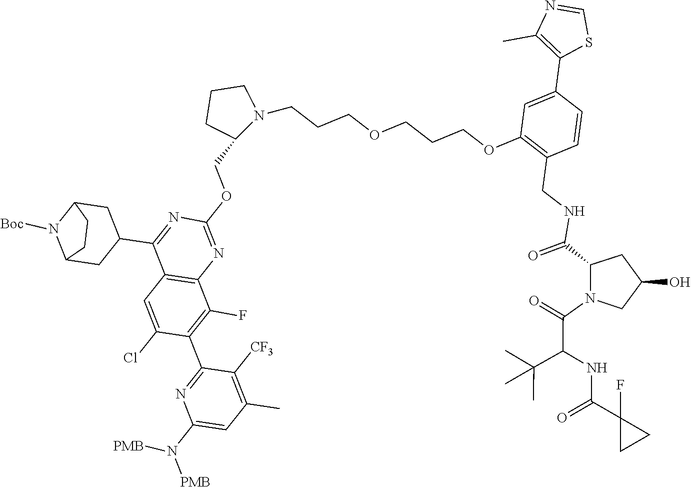

A mixture of (2S,4R)-1-[(2R)-2-[(1-fluorocyclopropanecarbonyl)amino]-3,3-dimethyl-butanoyl]-4-hydroxy-N-[[2-hydroxy-4-(4-methylthiazol-5-yl)phenyl]methyl]pyrrolidine-2-carboxamide (36 mg, 0.068 mmol, 1 eq), tert-butyl 3-[7-[6-[bis[(4-methoxyphenyl) methyl]amino]-4-methyl-3-(trifluoromethyl)-2-pyridyl]-6-chloro-8-fluoro-2-[[(2S)-1-[3-[3-(p-tolylsulfonyloxy)propoxy]propyl]pyrrolidin-2-yl]methoxy]quinazolin-4-yl]-3,8-diazabicyclo[3.2.1]octane-8-carboxylate (80 mg, 0.068 mmol, 1 eq), potassium carbonate (28 mg, 0.20 mmol, 3 eq), potassium iodide (3 mg) in N,N-dimethylformamide (1 mL) was stirred at 70° C. for 2 hours. LCMS showed the desired mass was detected. The reaction mixture was diluted with water (10 mL) and extracted with ethyl acetate (10 mL×3). The combined organic layers were washed with brine (5 mL×2), dried over anhydrous sodium sulfate, filtered and concentrated under reduced pressure to give a residue which was purified by prep-TLC (silicon dioxide, dichloromethane/methanol=10/1 and then petroleum ether/ethyl acetate=0/1) to afford the desired product (60 mg, 0.039 mmol, 57% yield) as a white solid. LCMS: (ESI, m/z): 1537.7 [M+1]⁺.

Step 2: (2S,4R)—N-[[2-[3-[3-[(2S)-2-[[7-[6-amino-4-methyl-3-(trifluoromethyl)-2-pyridyl]-6-chloro-4-(3,8-diazabicyclo[3.2.1]octan-3-yl)-8-fluoro-quinazolin-2-yl]oxymethyl]pyrrolidin-1-yl]propoxy]propoxy]-4-(4-methylthiazol-5-yl)phenyl]methyl]-1-[(2R)-2-[(1-fluorocyclopropanecarbonyl)amino]-3,3-dimethyl-butanoyl]-4-hydroxy-pyrrolidine-2-carboxamide (Example 3)

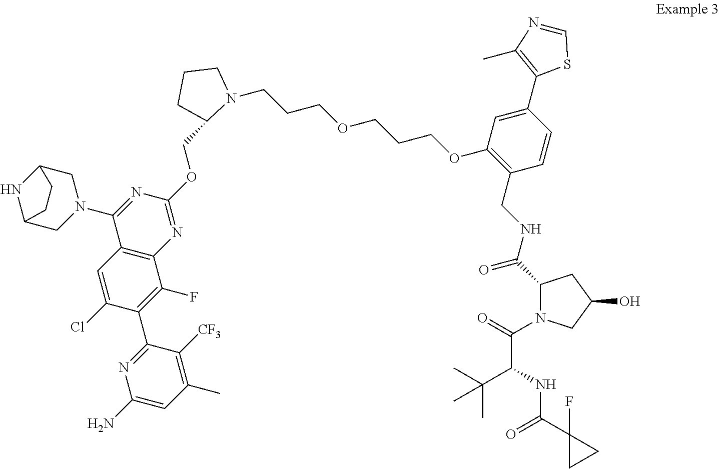 [SMILES: Cc1cc(N)nc(-c2c(Cl)cc3c(N4CC5CCC(C4)N5)nc(OC[C@@H]4CCCN4CCCOCCCOc4cc(-c5scnc5C)ccc4CNC(=O)[C@@H]4C[C@@H](O)CN4C(=O)[C@H](NC(=O)C4(F)CC4)C(C)(C)C)nc3c2F)c1C(F)(F)F]

To a solution of tert-butyl 3-[7-[6-[bis[(4-methoxyphenyl)methyl]amino]-4-methyl-3-(trifluoromethyl)-2-pyridyl]-6-chloro-8-fluoro-2-[[(2S)-1-[3-[3-[2-[[[(2S,4R)-1-[(2R)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]propoxy]propyl]pyrrolidin-2-yl]methoxy]quinazolin-4-yl]-3,8-diazabicyclo[3.2.1]octane-8-carboxylate (60 mg, 0.039 mmol, 1 eq) in dichloromethane (1 mL) was added trifluoroacetic acid (0.5 mL). Then trifluoromethanesulfonic acid (0.025 mL) was added into the mixture at 0° C. and the resulting reaction solution was stirred at 0° C. for 0.5 hour. LCMS showed the desired mass was detected. The mixture was concentrated under reduced pressure to get a residue which was purified by prep-HPLC (column: Phenomenex Synergi C18 150*25 mm*10 um; mobile phase: [water(0.225% formic acid)-acetonitrile]; B %:16%-36%, 10 min) to generate the desired product (11.11 mg, 0.0085 mmol, 22% yield, 98% purity, formate) as a white solid. LCMS: (ESI, m/z): 1196.4 [M]⁺. ¹H NMR: (400 MHz, DMSO-d₆) δ: 8.94 (s, 1H), 8.23-8.16 (m, 2H), 7.79 (s, 1H), 7.42-7.33 (m, 1H), 7.26 (d, J=7.6 Hz, 1H), 6.98-6.92 (m, 2H), 6.83 (s, 2H), 6.48 (s, 1H), 4.59-4.50 (m, 1H), 4.43 (t, J=7.6 Hz, 1H), 4.38-4.28 (m, 4H), 4.26-3.98 (m, 6H), 3.69 (d, J=4.0 Hz, 3H), 3.60 (d, J=1.2 Hz, 4H), 3.03-3.00 (m, 1H), 2.92-2.87 (m, 1H), 2.76 (d, J=2.4 Hz, 1H), 2.42 (s, 3H), 2.38-2.28 (m, 5H), 2.19-2.02 (m, 3H), 1.99-1.79 (m, 5H), 1.75-1.56 (m, 10H), 1.38-1.28 (m, 1H), 1.25-1.15 (m, 2H), 1.11-1.03 (m, 1H), 0.99 (s, 9H).

Example 4: DHC-101, (2S,4R)-1-[(2S)-2-[3-[3-[(2S)-2-[[7-(2-amino-7-fluoro-1,3-benzothiazol-4-yl)-6-chloro-4-(3,8-diazabicyclo[3.2.1]octan-3-yl)-8-fluoro-quinazolin-2-yl]oxymethyl]pyrrolidin-1-yl]propoxy]propanoylamino]-3,3-dimethylbutanoyl]-4-hydroxy-N-[(1S)-1-[4-(4-methylthiazol-5-yl)phenyl]ethyl]pyrrolidine-2-carboxamide

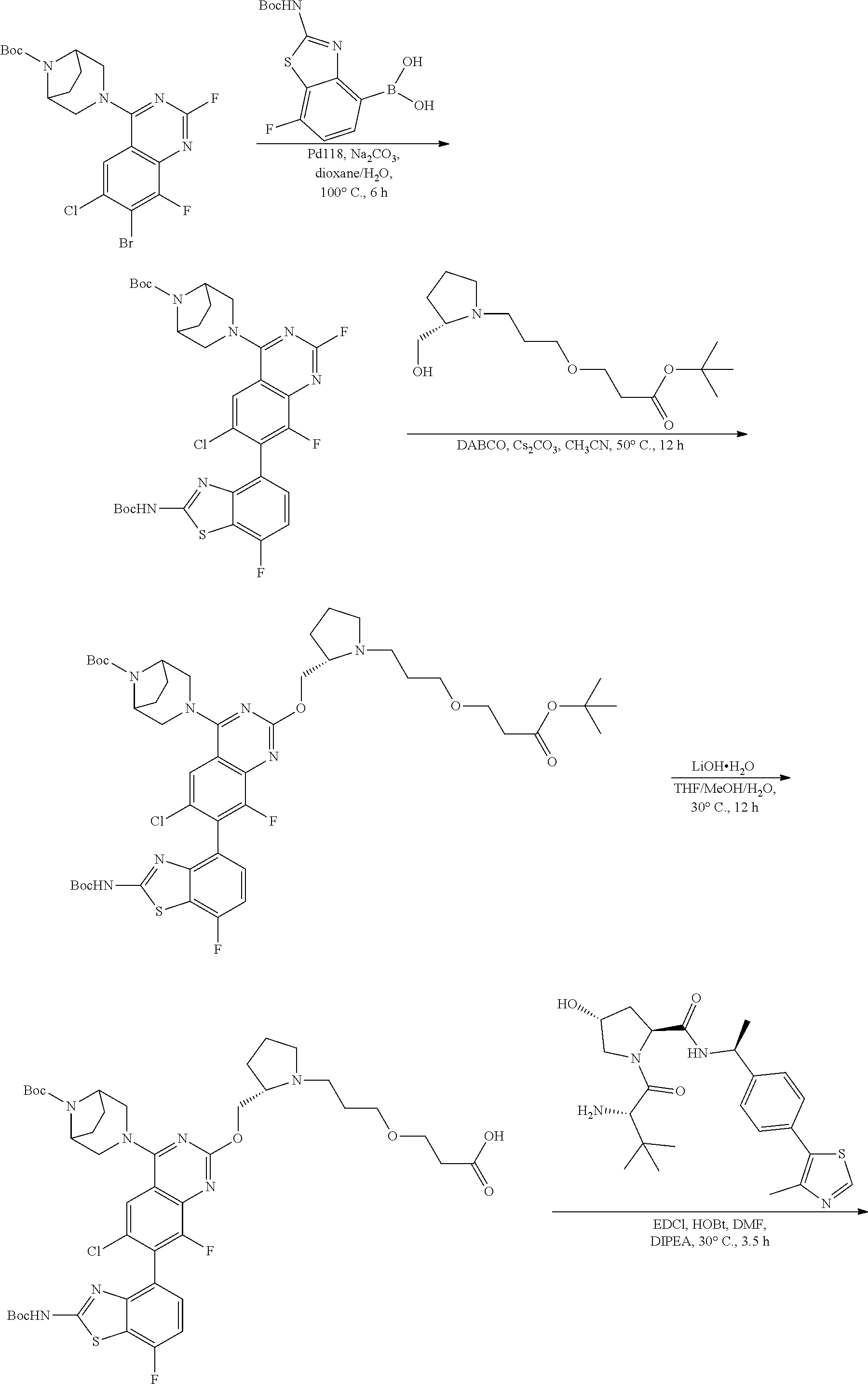

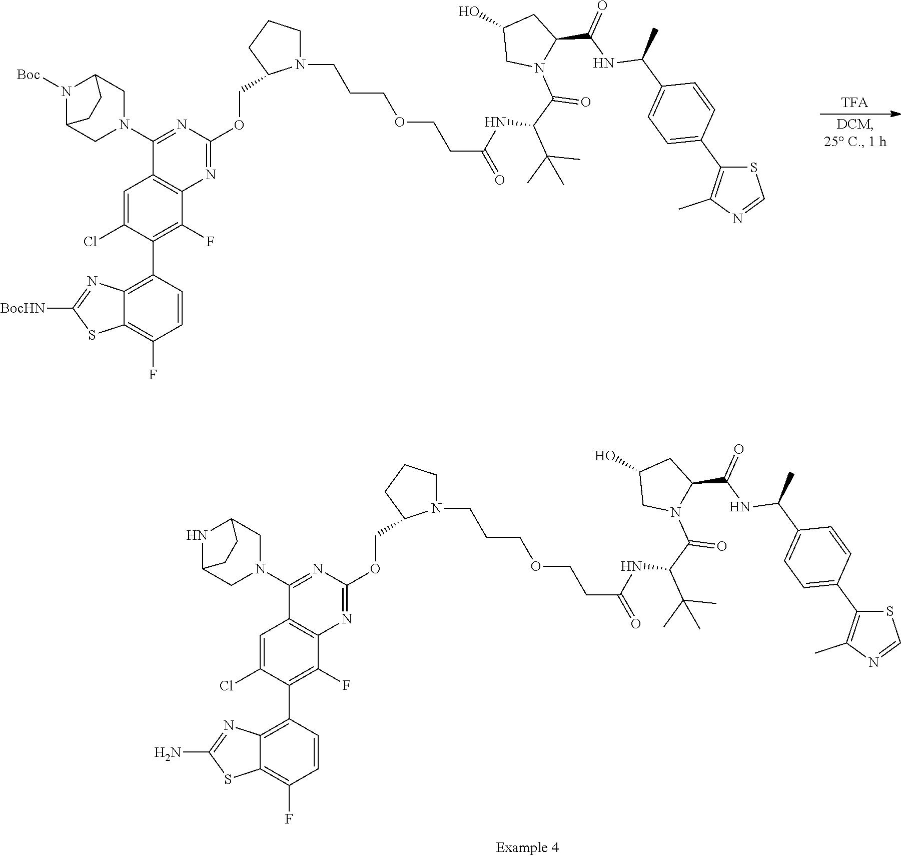 [SMILES: Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4C(=O)OC(C)(C)C)c3cc(Cl)c(-c4ccc(F)c5sc(NC(=O)OC(C)(C)C)nc45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cc(Cl)c(-c4ccc(F)c5sc(N)nc45)c(F)c3n2)C(C)(C)C)cc1]

Step 1: tert-butyl 3-[7-[2-(tert-butoxycarbonylamino)-7-fluoro-1,3-benzothiazol-4-yl]-6-chloro-2,8-difluoro-quinazolin-4-yl]-3,8-diazabicyclo[3.2.1]octane-8-carboxylate

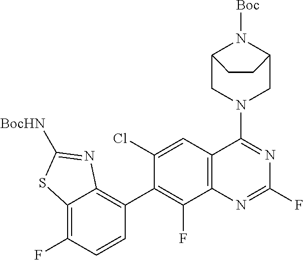 [SMILES: CC(C)(C)OC(=O)Nc1nc2c(-c3c(Cl)cc4c(N5CC6CCC(C5)N6C(=O)OC(C)(C)C)nc(F)nc4c3F)ccc(F)c2s1]

To a solution of tert-butyl 3-(7-bromo-6-chloro-2,8-difluoro-quinazolin-4-yl)-3,8-diazabicyclo[3.2.1]octane-8-carboxylate (1 g, 2.04 mmol, 1 eq) and [2-(tert-butoxycarbonylamino)-7-fluoro-1,3-benzothiazol-4-yl]boronic acid (956 mg, 3.06 mmol, 1.5 eq) in dioxane (20 mL) and water (4 mL), was added sodium carbonate (649 mg, 6.13 mmol, 3 eq) and ditertbutyl(cyclopentyl)phosphane; dichloropalladium; iron (266 mg, 0.41 mmol, 0.2 eq). The mixture was stirred at 100° C. for 6 hours under the protection of nitrogen. LCMS showed that the reaction was completed. The combined mixture was diluted with water (20 mL) before extracted with ethyl acetate (20 mL×3). The combined organic layers were washed with brine (15 mL), dried over anhydrous sodium sulfate, filtered and concentrated under reduced pressure to get a residue which was purified by silica gel column chromatography (petroleum ether: ethyl acetate=8:1) to afford the desired product (800 mg, 1.18 mmol, 57% yield) as a yellow solid. LCMS: (ESI, m/z): 677.1 [M]⁺. ¹H NMR: (400 MHz, DMSO-d₆) δ: 12.15 (s, 1H), 8.11 (s, 1H), 7.45 (dd, J=5.6, 8.4 Hz, 1H), 7.37-7.30 (m, 1H), 4.54-4.35 (m, 2H), 4.29 (d, J=10.8 Hz, 2H), 3.73 (d, J=12.4 Hz, 1H), 3.63 (d, J=12.4 Hz, 1H), 1.82 (s, 2H), 1.70-1.62 (m, 2H), 1.47 (s, 18H).

Step 2: tert-butyl 3-[7-[2-(tert-butoxycarbonylamino)-7-fluoro-1,3-benzothiazol-4-yl]-2-[[(2S)-1-[3-(3-tert-butoxy-3-oxopropoxy)propyl]pyrrolidin-2-yl]methoxy]-6-chloro-8-fluoro-quinazolin-4-yl]-3,8-diazabicyclo[3.2.1]octane-8-carboxylate

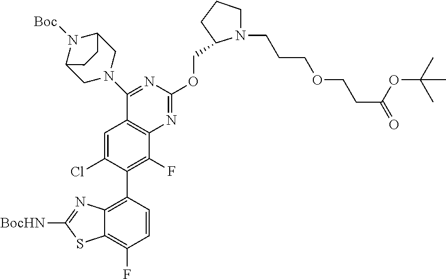 [SMILES: CC(C)(C)OC(=O)CCOCCCN1CCC[C@H]1COc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of tert-butyl 3-[7-[2-(tert-butoxycarbonylamino)-7-fluoro-1,3-benzothiazol-4-yl]-6-chloro-2,8-difluoro-quinazolin-4-yl]-3,8-diazabicyclo[3.2.1]octane-8-carboxylate (190 mg, 0.28 mmol, 1 eq) and tert-butyl 3-[3-[(2S)-2-(hydroxymethyl)pyrrolidin-1-yl]propoxy]propanoate (162 mg, 0.56 mmol, 2 eq) in acetonitrile (4 mL) was added cesium carbonate (183 mg, 0.56 mmol, 2 eq) and 1,4-diazabicyclo[2.2.2]octane (3 mg, 0.03 mmol, 0.1 eq). The mixture was stirred at 50° C. for 12 hours. LCMS showed that the reaction completed. The mixture was filtered and the filtrate was concentrated under reduced pressure to get a residue which was purified by prep-TLC (dichloromethane/methanol=10/1) to give the desired product (80 mg, 0.084 mmol, 29% yield, 98% purity) as a yellow solid. LCMS: (ESI, m/z): 944.5 [M]⁺.

Step 3: 3-[3-[(2S)-2-[[7-[2-(tert-butoxycarbonylamino)-7-fluoro-1,3-benzothiazol-4-yl]-4-(8-tert-butoxycarbonyl-3,8-diazabicyclo[3.2.1]octan-3-yl)-6-chloro-8-fluoro-quinazolin-2-yl]oxymethyl]pyrrolidin-1-yl]propoxy]propanoic acid

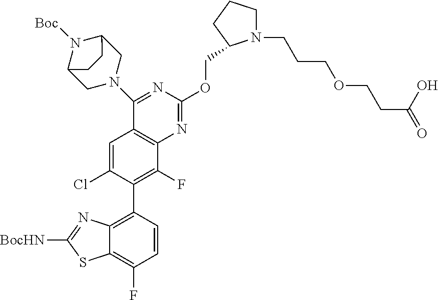 [SMILES: CC(C)(C)OC(=O)Nc1nc2c(-c3c(Cl)cc4c(N5CC6CCC(C5)N6C(=O)OC(C)(C)C)nc(OC[C@@H]5CCCN5CCCOCCC(=O)O)nc4c3F)ccc(F)c2s1]

To a solution of tert-butyl 3-[7-[2-(tert-butoxycarbonylamino)-7-fluoro-1,3-benzothiazol-4-yl]-2-[[(2S)-1-[3-(3-tert-butoxy-3-oxo-propoxy)propyl]pyrrolidin-2-yl]methoxy]-6-chloro-8-fluoro-quinazolin-4-yl]-3,8-diazabicyclo[3.2.1]octane-8-carboxylate (80 mg, 0.084 mmol, 1 eq) in the mixed solvent of tetrahydrofuran (0.5 mL), methanol (0.5 mL) and water (0.3 mL), was added lithium hydroxide (36 mg, 0.84 mmol, 10 eq). The mixture was stirred at 30° C. for 12 hours. LCMS showed that the desired mass was detected. The reaction mixture was concentrated under reduced pressure to get a yellow solid. The solid was dissolved in water (5 mL), hydrogen chloride solution (0.1 M) was used to adjust the pH to 6 before extracted by ethyl acetate (20 mL×3), and the combined organic layers were dried over anhydrous sodium sulfate, filtered and concentrated to give the residue which was purified by prep-TLC (dichloromethane/methanol=8/1) to afford the desired product (30 mg, 0.033 mmol, 39% yield, 98% purity) as a white solid. LCMS: (ESI, m/z): 888.5 [M]⁺.

Step 4: tert-butyl 3-[7-[2-(tert-butoxycarbonylamino)-7-fluoro-1,3-benzothiazol-4-yl]-6-chloro-8-fluoro-2-[[(2S)-1-[3-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethylpropyl]amino]-3-oxo-propoxy]propyl]pyrrolidin-2-yl]methoxy]quinazolin-4-yl]-3,8-diazabicyclo[3.2.1]octane-8-carboxylate

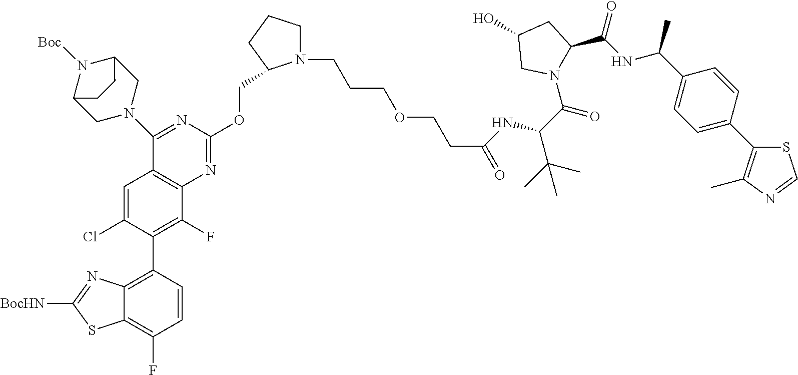 [SMILES: Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4C(=O)OC(C)(C)C)c3cc(Cl)c(-c4ccc(F)c5sc(NC(=O)OC(C)(C)C)nc45)c(F)c3n2)C(C)(C)C)cc1]

To a solution of 3-[3-[(2S)-2-[[7-[2-(tert-butoxycarbonylamino)-7-fluoro-1,3-benzothiazol-4-yl]-4-(8-tert-butoxycarbonyl-3,8-diazabicyclo[3.2.1]octan-3-yl)-6-chloro-8-fluoro-quinazolin-2-yl]oxymethyl]pyrrolidin-1-yl]propoxy]propanoic acid (30 mg, 0.033 mmol, 1 eq) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (30 mg, 0.067 mmol, 2 eq) in N,N-dimethylformamide (1 mL), was added N,N-diisopropylethylamine (22 mg, 0.17 mmol, 5 eq) and 1-hydroxybenzotriazole (10 mg, 0.067 mmol, 2 eq). The mixture was stirred at 30° C. for 0.5 hour. Then, N-(3-dimethylaminopropyl)-N-ethylcarbodiimide hydrochloride (13 mg, 0.067 mmol, 2 eq) was added, and the mixture was stirred at 30° C. for 3 hours. LCMS showed that the desired mass was detected. Ethyl acetate (10 mL) and water (10 mL) were added before the mixture was extracted by ethyl acetate (10 mL×3). The combined organic layers were washed with brine (10 mL×3), dried over anhydrous sodium sulfate, filtered and concentrated under reduced pressure to get the residue which was purified by prep-TLC (dichloromethane/methanol=10/1) to generate the desired product (22 mg, 0.016 mmol, 46% yield, 93% purity) as a white solid. LCMS: (ESI, m/z): 1314.8 [M]⁺.

Step 5: (2S,4R)-1-[(2S)-2-[3-[3-[(2S)-2-[[7-(2-amino-7-fluoro-1,3-benzothiazol-4-yl)-6-chloro-4-(3,8-diazabicyclo[3.2.1]octan-3-yl)-8-fluoro-quinazolin-2-yl]oxymethyl]pyrrolidin-1-yl]propoxy]propanoylamino]-3,3-dimethylbutanoyl]-4-hydroxy-N-[(1S)-1-[4-(4-methylthiazol-5-yl)phenyl]ethyl]pyrrolidine-2-carboxamide

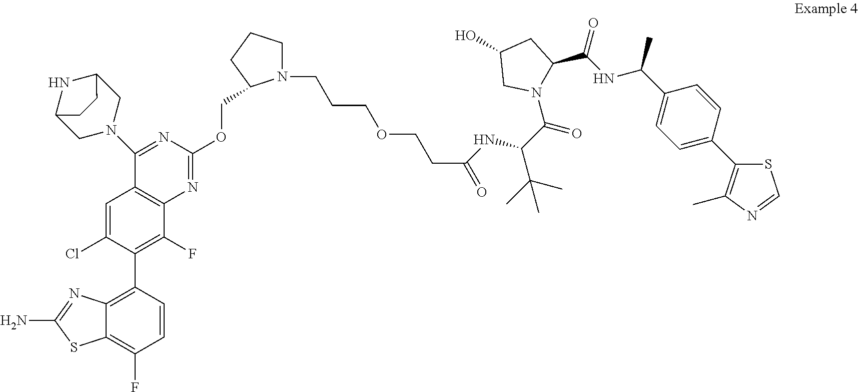 [SMILES: Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cc(Cl)c(-c4ccc(F)c5sc(N)nc45)c(F)c3n2)C(C)(C)C)cc1]

To a solution of tert-butyl 3-[7-[2-(tert-butoxycarbonylamino)-7-fluoro-1,3-benzothiazol-4-yl]-6-chloro-8-fluoro-2-[[(2S)-1-[3-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]propyl]pyrrolidin-2-yl]methoxy]quinazolin-4-yl]-3,8-diazabicyclo[3.2.1]octane-8-carboxylate (20 mg, 0.015 mmol, 1 eq) in dichloromethane (3 mL) was added trifluoroacetic acid (1 mL). The mixture was stirred at 25° C. for 1 hour. LCMS showed that the desired mass was detected. The reaction mixture was concentrated under reduced pressure to get a residue which was purified by prep-HPLC (column: Phenomenex Synergi C18 150*25 mm*10 um; mobile phase: [water(FA)-ACN]; B %: 13%-43%, 10 min) to afford the desired product (9.63 mg, 0.008 mmol, 52% yield, 100% purity, formate[2]) as a white solid. LCMS: (ESI, m/z): 557.8 [M/2]⁺. ¹H NMR: (400 MHz, DMSO-d₆) δ: 9.08-8.91 (m, 1H), 8.37 (d, J=7.2 Hz, 1H), 8.30-8.18 (m, 2H), 8.01-7.73 (m, 4H), 7.49-7.30 (m, 4H), 7.27-7.16 (m, 1H), 7.11-6.96 (m, 1H), 4.93-4.82 (m, 1H), 4.56-4.40 (m, 2H), 4.37-4.25 (m, 4H), 4.14-3.98 (m, 2H), 3.89-3.74 (m, 3H), 3.17-2.99 (m, 4H), 2.91-2.76 (m, 3H), 2.49-2.43 (m, 5H), 2.36-2.11 (m, 5H), 2.05-1.81 (m, 3H), 1.78-1.57 (m, 10H), 1.36 (d, J=6.8 Hz, 3H), 0.95-0.79 (m, 9H).

Example 5: 3-[4-[1-[7-[(2S)-2-[[7-[6-amino-4-methyl-3-(trifluoromethyl)-2-pyridyl]-6-chloro-4-(3,8-diazabicyclo[3.2.1]octan-3-yl)-8-fluoro-quinazolin-2-yl]oxymethyl]pyrrolidin-1-yl]heptyl]-4-piperidyl]-6-fluoro-1-oxo-isoindolin-2-yl]piperidine-2,6-dione

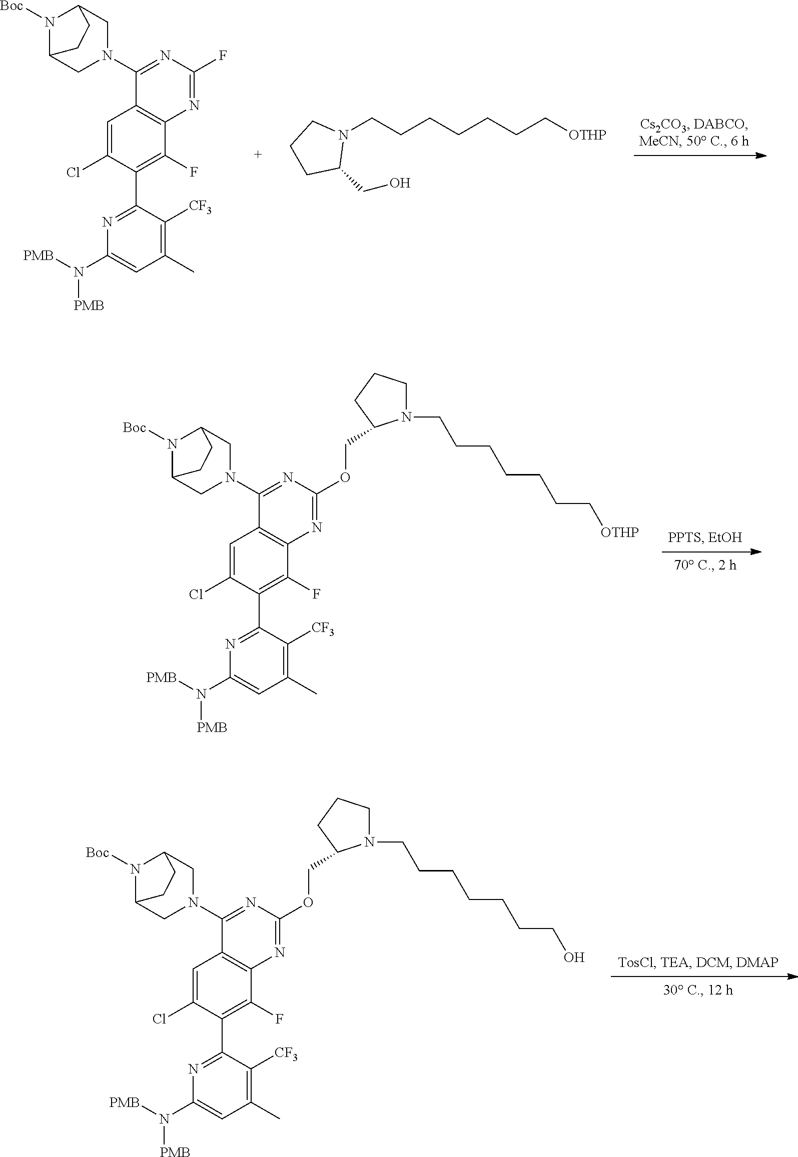

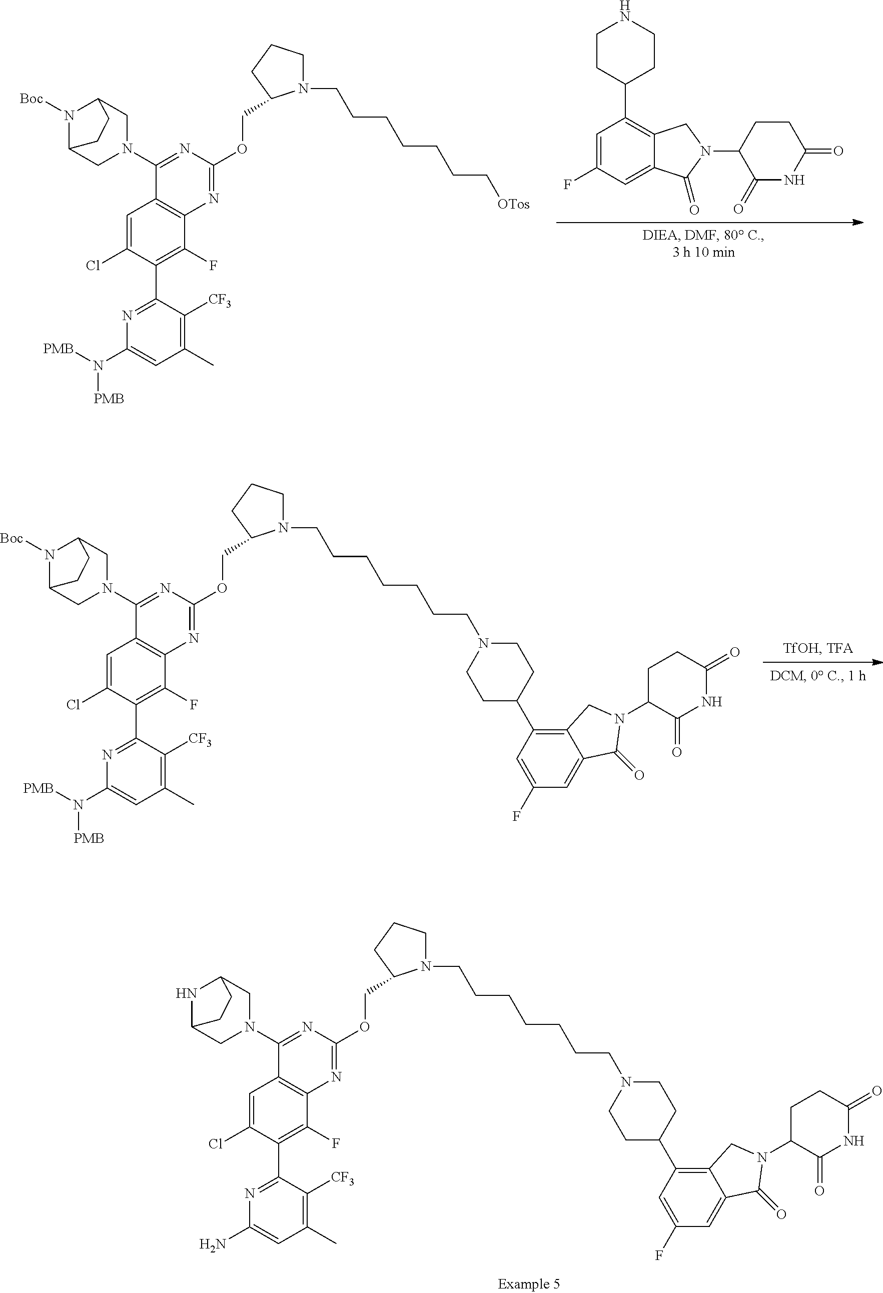

Step 1: tert-butyl 3-[7-[6-[bis[(4-methoxyphenyl)methyl]amino]-4-methyl-3-(trifluoromethyl)-2-pyridyl]-6-chloro-8-fluoro-2-[[(2S)-1-(7-tetrahydropyran-2-yloxyheptyl)pyrrolidin-2-yl]methoxy]quinazolin-4-yl]-3,8-diazabicyclo[3.2.1]octane-8-carboxylate

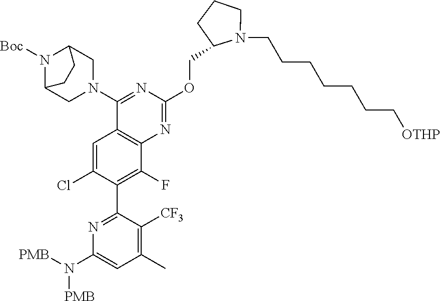

To a solution of [(2S)-1-(7-tetrahydropyran-2-yloxyheptyl)pyrrolidin-2-yl]methanol (290 mg, 0.97 mmol, 2 eq) and tertbutyl3-[7-[6-[bis[(4-methoxyphenyl)methyl]amino]-4-methyl-3-(trifluoromethyl)-2-pyridyl]-6-chloro-2,8-difluoro-quinazolin-4-yl]-3,8-diazabicyclo[3.2.1]octane-8-carboxylate (400 mg, 0.48 mmol, 1 eq) in acetonitrile (4 mL), was added cesium carbonate (315 mg, 0.97 mmol, 2 eq) and 1,4-diazabicyclo[2.2.2]octane (5 mg, 0.05 mmol, 0.1 eq). The mixture was stirred at 50° C. for 6 h. LCMS showed that the desired mass was detected. The mixture was filtered and the filter was concentrated under reduced pressure to get a residue which was purified by prep-TLC (dichloromethane/methanol=15/1) to afford the desired product (140 mg, 0.13 mmol, 26% yield) as a yellow solid. LCMS: (ESI, m/z): 552.8[M/2]⁺.

Step 2: tert-butyl 3-[7-[6-[bis[(4-methoxyphenyl)methyl]amino]-4-methyl-3-(trifluoromethyl)-2-pyridyl]-6-chloro-8-fluoro-2-[[(2S)-1-(7-hydroxyheptyl)pyrrolidin-2-yl]methoxy]quinazolin-4-yl]-3,8-diazabicyclo[3.2.1]octane-8-carboxylate

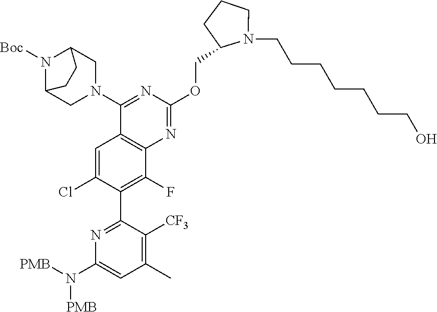

To a solution of tert-butyl 3-[7-[6-[bis[(4-methoxyphenyl)methyl]amino]-4-methyl-3-(trifluoromethyl)-2-pyridyl]-6-chloro-8-fluoro-2-[[(2S)-1-(7-tetrahydropyran-2-yloxyheptyl)pyrrolidin-2-yl]methoxy]quinazolin-4-yl]-3,8-diazabicyclo[3.2.1]octane-8-carboxylate (190 mg, 0.17 mmol, 1 eq) in ethanol (4 mL), was added 4-methylbenzenesulfonic acid; pyridine (87 mg, 0.34 mmol, 2 eq). The mixture was stirred at 70° C. for 2 hours. LCMS showed that the desired mass was detected. The reaction mixture was concentrated under reduced pressure to get a residue which was purified by prep-TLC (dichloromethane/methanol=10/1) to generate the desired product (130 mg, 0.13 mmol, 74% yield) as a yellow solid. LCMS: (ESI, m/z): 1020.1[M]⁺.

Step 3: tert-butyl 3-[7-[6-[bis[(4-methoxyphenyl)methyl]amino]-4-methyl-3-(trifluoromethyl)-2-pyridyl]-6-chloro-8-fluoro-2-[[(2S)-1-[7-(p-tolylsulfonyloxy)heptyl]pyrrolidin-2-yl]methoxy]quinazolin-4-yl]-3,8-diazabicyclo[3.2.1]octane-8-carboxylate

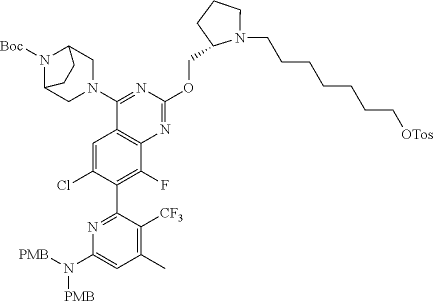

To a solution of tert-butyl 3-[7-[6-[bis[(4-methoxyphenyl)methyl]amino]-4-methyl-3-(trifluoromethyl)-2-pyridyl]-6-chloro-8-fluoro-2-[[(2S)-1-(7-hydroxyheptyl)pyrrolidin-2-yl]methoxy]quinazolin-4-yl]-3,8-diazabicyclo[3.2.1]octane-8-carboxylate (130 mg, 0.13 mmol, 1 eq) in dichloromethane (3 mL), was added triethylamine (39 mg, 0.38 mmol, 3 eq), p-toluenesulfonyl chloride (49 mg, 0.25 mmol, 2 eq) and dimethylaminopyridine (2 mg, 0.013 mmol, 0.1 eq). The mixture was stirred at 30° C. for 12 hours. TLC (dichloromethane/methanol=10/1) showed that the reaction was completed. Dichloromethane (10 mL) and water (15 mL) were added before the mixture extracted with dichloromethane (10 mL×3). The combined organic layers were washed with brine (10 mL), dried over anhydrous sodium sulfate, filtered and concentrated under reduced pressure to get the residue which was purified by prep-TLC (dichloromethane/methanol=10/1) to give the desired product (110 mg, 0.09 mmol, 71% yield, 97% purity) as a yellow solid. LCMS: (ESI, m/z): 1174.7 [M]⁺.

Step 4: tert-butyl 3-[7-[6-[bis[(4-methoxyphenyl)methyl]amino]-4-methyl-3-(trifluoromethyl)-2-pyridyl]-6-chloro-2-[[(2S)-1-[7-[4-[2-(2,6-dioxo-3-piperidyl)-6-fluoro-1-oxo-isoindolin-4-yl]-1-piperidyl]heptyl]pyrrolidin-2-yl]methoxy]-8-fluoroquinazolin-4-yl]-3,8-diazabicyclo[3.2.1]octane-8-carboxylate

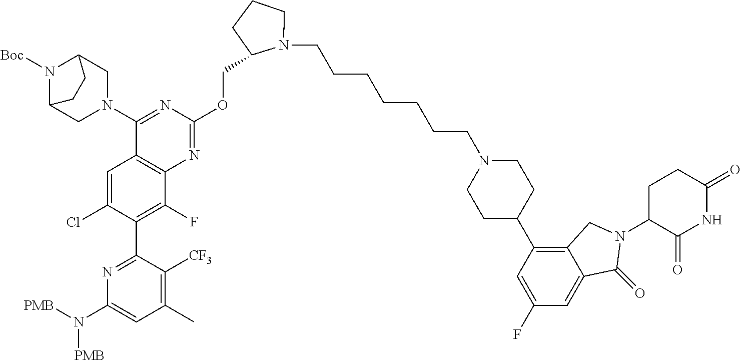

To a solution of 3-[6-fluoro-1-oxo-4-(4-piperidyl)isoindolin-2-yl]piperidine-2,6-dione (47 mg, 0.102 mmol, 1.2 eq, trifluoroacetate) in N,N-dimethylformamide (3 mL), was added N,N-diisopropylethylamine (55 mg, 0.42 mmol, 5 eq), and stirred at 25° C. for 10 min. Then, tert-butyl 3-[7-[6-[bis[(4-methoxyphenyl)methyl]amino]-4-methyl-3-(trifluoromethyl)-2-pyridyl]-6-chloro-8-fluoro-2-[[(2S)-1-[7-(ptolylsulfonyloxy)heptyl]pyrrolidin-2-yl]methoxy]quinazolin-4-yl]-3,8-diazabicyclo[3.2.1]octane-8-carboxylate (100 mg, 0.085 mmol, 1 eq) and potassium iodide (2 mg, 0.009 mmol, 0.1 eq) was added, the resulting mixture was stirred at 80° C. for 3 hours. LCMS showed that the desired mass was detected. Ethyl acetate (15 mL) and water (15 mL) were added before the mixture was extracted with ethyl acetate (15 mL×3). The combined organic layers were washed with brine (15 mL×3), dried over anhydrous sodium sulfate, filtered and concentrated under reduced pressure to get a residue which was purified by prep-TLC (dichloromethane/methanol=10/1) to generate the desired product (54 mg, 0.037 mmol, 44% yield, 94% purity) as a yellow solid. LCMS: MS (ESI, m/z): 1347.9 [M]⁺.

Step 5: 3-[4-[1-[7-[(2S)-2-[[7-[6-amino-4-methyl-3-(trifluoromethyl)-2-pyridyl]-6-chloro-4-(3,8-diazabicyclo[3.2.1]octan-3-yl)-8-fluoro-quinazolin-2-yl]oxymethyl]pyrrolidin-1-yl]heptyl]-4-piperidyl]-6-fluoro-1-oxo-isoindolin-2-yl]piperidine-2,6-dione (Example 5)

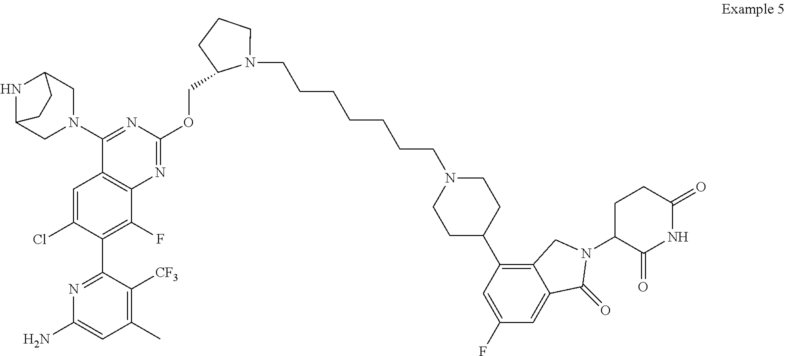 [SMILES: Cc1cc(N)nc(-c2c(Cl)cc3c(N4CC5CCC(C4)N5)nc(OC[C@@H]4CCCN4CCCCCCCN4CCC(c5cc(F)cc6c5CN(C5CCC(=O)NC5=O)C6=O)CC4)nc3c2F)c1C(F)(F)F]

To a solution of tert-butyl 3-[7-[6-[bis[(4-methoxyphenyl)methyl]amino]-4-methyl-3-(trifluoromethyl)-2-pyridyl]-6-chloro-2-[[(2S)-1-[7-[4-[2-(2,6-dioxo-3-piperidyl)-6-fluoro-1-oxo-isoindolin-4-yl]-1-piperidyl]heptyl]pyrrolidin-2-yl]methoxy]-8-fluoroquinazolin-4-yl]-3,8-diazabicyclo[3.2.1]octane-8-carboxylate (50 mg, 0.037 mmol, 1 eq) in dichloromethane (2 mL), was added trifluoroacetic acid (0.5 mL) and trifluoromethanesulfonic acid (43 mg, 0.28 mmol, 7.63 eq). The mixture was stirred at 0° C. for 1 hour. LCMS showed that the desired mass was detected. The mixture was purged by nitrogen at 0° C. to remove the solvent to get a residue which was purified by prep-HPLC (column: Phenomenex Synergi C18 150*25 mm*10 um; mobile phase: [water(FA)-ACN]; B %: 3%-33%, 10 min) to afford the desired product (15.63 mg, 0.013 mmol, 34% yield, 98% purity, formate[4]) as a white solid. LCMS: (ESI, m/z): 504.9 [M/2+1]⁺. ¹H NMR: (400 MHz, DMSO-d₆) δ: δ=11.04 (s, 1H), 8.28 (s, 4H), 7.92-7.69 (m, 1H), 7.50-7.25 (m, 2H), 6.91-6.70 (m, 2H), 6.48 (s, 1H), 5.13 (dd, J=5.2, 13.2 Hz, 1H), 4.57-4.42 (m, 2H), 4.40-4.27 (m, 4H), 4.26-4.18 (m, 2H), 4.12-4.05 (m, 2H), 2.94 (d, J=11.2 Hz, 4H), 2.90-2.75 (m, 4H), 2.61 (d, J=1.6 Hz, 2H), 2.27-2.12 (m, 4H), 2.04-1.84 (m, 5H), 1.78-1.57 (m, 12H), 1.47-1.35 (m, 4H), 1.31-1.12 (m, 7H).

Synthesis of Intermediate 4: tert-butyl 3-[8-fluoro-7-[8-fluoro-3-(methoxymethoxy)-1-naphthyl]-2-methylsulfonyl-pyrido[4,3-d]pyrimidin-4-yl]-3,8-diazabicyclo[3.2.1]octane-8-carboxylate

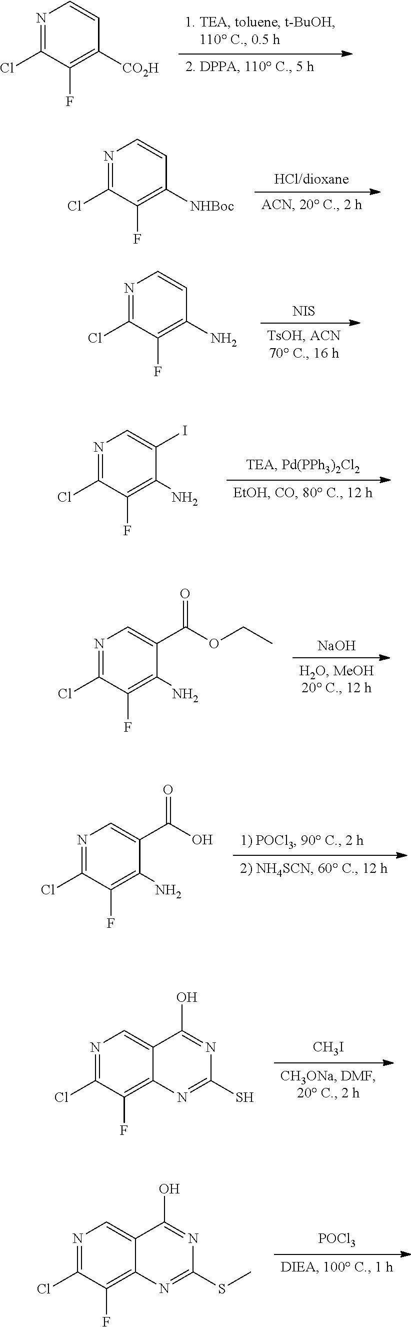

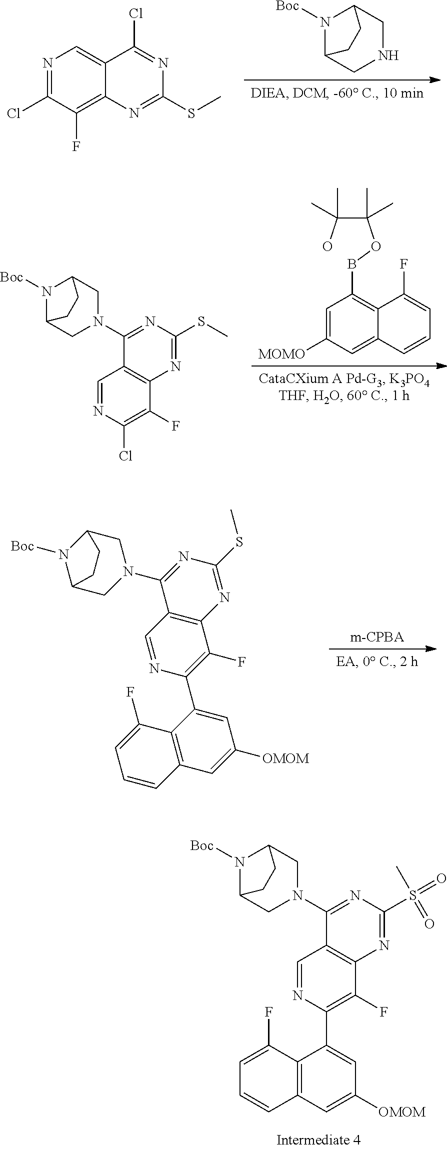

Step 1: tert-butyl N-(2-chloro-3-fluoro-4-pyridyl)carbamate

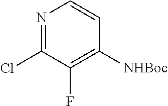 [SMILES: CC(C)(C)OC(=O)Nc1ccnc(Cl)c1F]

To a solution of 2-chloro-3-fluoro-pyridine-4-carboxylic acid (10 g, 56.97 mmol, 1 eq) in toluene (73 mL) was added triethylamine (17.29 g, 170.91 mmol, 3 eq) and tertiary butanol (56.16 g, 757.70 mmol, 13.3 eq). Then the mixture was stirred at 110° C. for 0.5 hour. The mixture was cooled to 25° C. and diphenylphosphoryl azide (23.52 g, 85.45 mmol, 1.5 eq) was added into the mixture. Then the mixture was stirred at 110° C. for additional 5 hours. The mixture was diluted with water (200 mL), extracted with ethyl acetate (200 mL×3), washed with brine (200 mL), dried over anhydrous sodium sulfate, filtered and concentrated under reduced pressure to give the crude product which was purified by column chromatography (silicon dioxide, petroleum ether/ethyl acetate=50/1 to 20/1) to afford the desired product (12.9 g, 52.30 mmol, 92% yield) as a yellow oil. LCMS: (ESI, m/z): 191.1 [M-56]⁺. ¹H NMR: (400 MHz, CDCl₃) δ: 8.14 (t, J=5.6 Hz, 1H), 8.02 (d, J=6.0 Hz, 1H), 1.54 (s, 9H).

Step 2: 2-chloro-3-fluoro-pyridin-4-amine

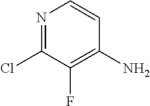

To a solution of tert-butyl N-(2-chloro-3-fluoro-4-pyridyl)carbamate (12 g, 48.65 mmol, 1 eq) in acetonitrile (120 mL) was added hydrogen chloride/dioxane (4 M, 50 mL). Then the mixture was stirred at 20° C. for 2 hours. The mixture was concentrated, diluted with water (30 mL), adjusted the pH with saturated aqueous sodium bicarbonate (50 mL) to about 9, extracted with ethyl acetate (200 mL×3). The combined organic layers were washed with brine (300 mL), dried over anhydrous sodium sulfate, filtered and concentrated under reduced pressure to give the crude product (7 g, 47.77 mmol, 98% yield) which was directly used for next step.

Step 3: 2-chloro-3-fluoro-5-iodo-pyridin-4-amine

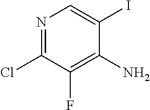

To a solution of 2-chloro-3-fluoro-pyridin-4-amine (2 g, 13.65 mmol, 1 eq) and N-iodosuccinimide (3.68 g, 16.38 mmol, 1.2 eq) in acetonitrile (10 mL) was added p-toluenesulfonic acid (130 mg, 0.68 mmol, 0.05 eq). Then the mixture was stirred at 70° C. for 12 hours. The mixture was diluted with water (50 mL), extracted with ethyl acetate (100 mL×3), quenched with saturated aqueous sodium sulfite (200 mL). The organic layer was washed with brine (100 mL), dried over anhydrous sodium sulfate, filtered and concentrated under reduced pressure to get a residue. The residue was purified by column chromatography (silicon dioxide, petroleum ether/ethyl acetate=100/1 to 5/1) to afford the desired product (2.7 g, 9.91 mmol, 72% yield) as a yellow solid. LCMS: (ESI, m/z): 273.2 [M+1]⁺. ¹H NMR: (400 MHz, MeOD) δ: 8.08 (s, 1H).

Step 4: ethyl 4-amino-6-chloro-5-fluoro-pyridine-3-carboxylate

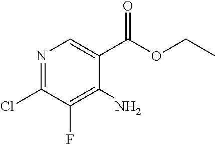

To a solution of 2-chloro-3-fluoro-5-iodo-pyridin-4-amine (1 g, 3.67 mmol, 1 eq) in ethyl alcohol (10 mL) was added triethylamine (1.34 g, 13.21 mmol, 3.6 eq) and [1,1′-bis(diphenylphosphino)ferrocene]dichloropalladium(ii) (257 mg, 0.37 mmol, 0.1 eq). Then the mixture was purged with nitrogen for 3 times. The mixture was stirred under carbon monoxide (50 Psi) at 80° C. for 12 hours. The mixture was filtered and then concentrated under reduced pressure to get a residue. The residue was purified by column chromatography (silicon dioxide, petroleum ether/ethyl acetate=10/1 to 3/1) to give the desired product (580 mg, 2.65 mmol, 72% yield) as a yellow solid. LCMS: (ESI, m/z): 219.4 [M+1]⁺. ¹H NMR: (400 MHz, DMSO-d₆) δ: 8.38 (s, 1H), 7.60 (s, 2H), 4.38-4.26 (m, 2H), 1.32 (t, J=7.2 Hz, 3H).

Step 5: 4-amino-6-chloro-5-fluoronicotinic acid

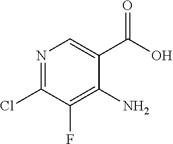 [SMILES: Nc1c(C(=O)O)cnc(Cl)c1F]

To a solution of ethyl 4-amino-6-chloro-5-fluoronicotinate (5 g, 22.87 mmol, 1 eq) in tetrahydrofuran (45 mL) and water (20 mL) was added sodium hydroxide (9.15 g, 228.72 mmol, 10 eq). Then the mixture was stirred at 20° C. for 12 hours. The mixture was concentrated under vacuum to remove tetrahydrofuran, adjusted the pH with aqueous hydrochloric (2M) to about 2, filtered, and washed with water (50 mL). The filter cake was triturated by dichloromethane (20 mL) to give the desired product (3.9 g, 20.47 mmol, 89% yield) as a gray solid. LCMS: (ESI, m/z): 191.4 [M+1]⁺. ¹H NMR: (400 MHz, DMSO-d₆) δ: 8.36 (s, 1H), 7.61 (s, 3H).

Step 6: 7-chloro-8-fluoro-2-sulfanyl-pyrido[4,3-d]pyrimidin-4-ol

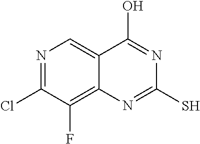 [SMILES: Oc1nc(S)nc2c(F)c(Cl)ncc12]

To a solution of 4-amino-6-chloro-5-fluoro-pyridine-3-carboxylic acid (2 g, 10.50 mmol, 1 eq) in phosphorus oxychloride (20 mL) was stirred at 90° C. for 2 hours. Then the mixture was concentrated under reduced pressure to remove phosphorus oxychloride and then the residue was dissolved in tetrahydrofuran (10 mL). A solution of ammonium thiocyanate (728 mg, 9.57 mmol, 2 eq) in tetrahydrofuran (10 mL) was added into the mixture at 20° C. Then the mixture was stirred at 60° C. for 12 hours. The mixture was diluted with water (30 mL), extracted with ethyl acetate (30 mL×3). The organic layers were washed with brine (30 mL), dried over anhydrous sodium sulfate, filtered and concentrated under reduced pressure to get a residue which was triturated with dichloromethane (10 mL) at 20° C. for 10 min to generate the desired product (1.1 g, 4.75 mmol, 99% yield) as a yellow solid. ¹H NMR: (400 MHz, DMSO-d₆) δ: 13.32 (s, 1H), 12.89 (s, 1H), 8.65 (s, 1H).

Step 7: 7-chloro-8-fluoro-2-methylsulfanyl-pyrido [4,3-d]pyrimidin-4-ol

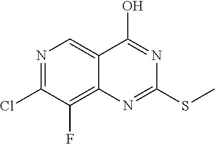

To a solution of 7-chloro-8-fluoro-2-sulfanyl-pyrido[4,3-d]pyrimidin-4-ol (5.9 g, 25.47 mmol, 1 eq) in N,N-dimethylformamide (30 mL) was added sodium methoxide (1.38 g, 25.47 mmol, 1 eq). The mixture was stirred at 20° C. for 10 minutes. Methyl iodide (3.62 g, 25.47 mmol, 1 eq) was added into the mixture and the stirring continued at 20° C. for 2 hours. The mixture was diluted with water (300 mL), extracted with ethyl acetate (200 ml×2). The combined organic layers were washed with brine (200 mL), dried over anhydrous sodium sulfate, filtered and concentrated under reduced pressure to generate a residue which was triturated with petroleum ether (50 mL) at 20° C. for 10 min to afford the desired product (6 g, 24.42 mmol, 95% yield) as a yellow solid. LCMS: (ESI, m/z): 246.3 [M+1]⁺. ¹H NMR: (400 MHz, DMSO-d₆) δ: 8.82 (s, 1H), 2.62 (s, 3H).

Step 8: 4,7-dichloro-8-fluoro-2-methylsulfanyl-pyrido[4,3-d] pyrimidine

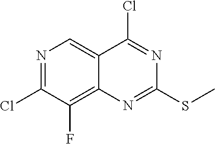

To a solution of 7-chloro-8-fluoro-2-methylsulfanyl-pyrido[4,3-d]pyrimidin-4-ol (5 g, 20.35 mmol, 1 eq) in phosphorus oxychloride (60 mL) was added N,N-diisopropylethylamine (5.26 g, 40.71 mmol, 2 eq). Then the mixture was stirred at 90° C. for 6 hours. The mixture was concentrated to generate a residue which was purified by column chromatography (silica dioxide, Petroleum ether/Ethyl acetate=100/1 to 10/1) to give the desired product (2.5 g, 9.47 mmol, 46% yield) as a white solid.

Step 9: tert-butyl 3-(7-chloro-8-fluoro-2-methylsulfanyl-pyrido[4,3-d]pyrimidin-4-yl)-3,8-diazabicyclo[3.2.1]octane-8-carboxylate

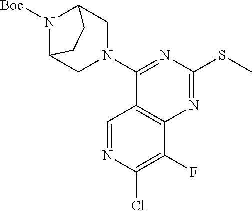 [SMILES: CSc1nc(N2CC3CCC(C2)N3C(=O)OC(C)(C)C)c2cnc(Cl)c(F)c2n1]

To a solution of 4,7-dichloro-8-fluoro-2-methylsulfanyl-pyrido[4,3-d]pyrimidine (2 g, 7.57 mmol, 1 eq) and N,N-diisopropylethylamine (4.89 g, 37.86 mmol, 6.60 mL, 5 eq) in dichloromethane (20 mL) was added tert-butyl 3,8-diazabicyclo[3.2.1]octane-8-carboxylate (1.61 g, 7.57 mmol, 1 eq) at −60° C. and stirred at −60° C. for 10 minutes. The mixture was diluted with water (20 mL), extracted with dichloromethane (20 mL×3). The combined organic layers were washed with brine (30 mL), dried over sodium sulfate, filtered and then concentrated under vacuum to generate a residue which was purified by column chromatography (silica dioxide, petroleum ether/ethyl acetate=10/1 to 3/1) to afford the desired product (2.68 g, 6.09 mmol, 80% yield) as a yellow solid. ¹H NMR: (400 MHz, DMSO-d₆) δ: 8.92 (s, 1H), 4.50 (d, J=12.4 Hz, 2H), 4.25 (s, 2H), 3.64 (d, J=12.8 Hz, 2H), 2.56 (s, 3H), 1.85-1.74 (m, 2H), 1.61 (d, J=7.6 Hz, 2H), 1.47 (s, 9H).

Step 10: tert-butyl 3-[8-fluoro-7-[8-fluoro-3-(methoxymethoxy)-1-naphthyl]-2-methylsulfanyl-pyrido[4,3-d]pyrimidin-4-yl]-3,8-diazabicyclo[3.2.1]octane-8-carboxylate

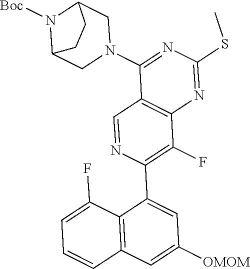

To a solution of tert-butyl 3-(7-chloro-8-fluoro-2-methylsulfanyl-pyrido[4,3-d]pyrimidin-4-yl)-3,8-diazabicyclo[3.2.1]octane-8-carboxylate (1.3 g, 2.95 mmol, 1 eq) and 2-[8-fluoro-3-(methoxymethoxy)-1-naphthyl]-4,4,5,5-tetramethyl-1,3,2-dioxaborolane (1.28 g, 3.84 mmol, 1.3 eq) in tetrahydrofuran (13 mL) was added potassium phosphate (1.5M, 5.9 mL, 3 eq). The mixture was purged with nitrogen for 3 times and [2-(2-aminophenyl) phenyl]palladium(1+); bis(1-adamantyl)-butyl-phosphane; methanesulfonate (215 mg, 0.3 mmol, 0.1 eq) was added into the mixture, the mixture was then stirred at 60° C. for 1 hour. The mixture was filtered and the filtrate was concentrated under vacuum to generate a residue which was purified by prep-TLC (silica dioxide, petroleum ether/ethyl acetate=2/1) to afford the desired product (700 mg, 1.15 mmol, 39% yield) as a yellow oil. LCMS: (ESI, m/z): 610.3[M+1]⁺.

Step 11: tert-butyl 3-[8-fluoro-7-[8-fluoro-3-(methoxymethoxy)-1-naphthyl]-2-methylsulfonyl-pyrido[4,3-d]pyrimidin-4-yl]-3,8-diazabicyclo[3.2.1]octane-8-carboxylate

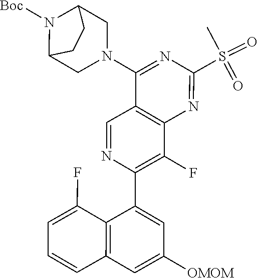

To a solution of tert-butyl 3-[8-fluoro-7-[8-fluoro-3-(methoxymethoxy)-1-naphthyl]-2-methylsulfanyl-pyrido[4,3-d]pyrimidin-4-yl]-3,8-diazabicyclo[3.2.1]octane-8-carboxylate (700 mg, 1.15 mmol, 1 eq) in ethyl acetate (10 mL) was added meta-chloroperbenzoic acid (699 mg, 3.44 mmol, 80% purity, 3 eq) at 0° C. Then the mixture was stirred at 0° C. for 2 hours. The mixture was quenched with saturated aqueous sodium sulfite (200 mL), extracted with ethyl acetate (100 mL×2). The combined organic layers were washed with brine (100 mL), dried over sodium sulfate, filtered and then concentrated under vacuum to generate a residue which was purified by prep-TLC (silica dioxide, petroleum ether/ethyl acetate=1/1) to afford the desired product (530 mg, 0.83 mmol, 72% yield) as a yellow oil. LCMS: (ESI, m/z): 642.3[M]⁺.

Synthesis of Intermediate 5: ethyl 5-[(2S)-2-(hydroxymethyl)pyrrolidin-1-yl]pentanoate

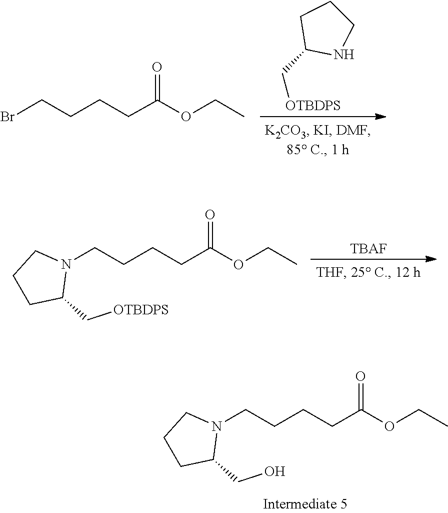

Step 1: ethyl 5-[(2S)-2-[[tert-butyl(diphenyl)silyl]oxymethyl] pyrrolidin-1-yl]pentanoate

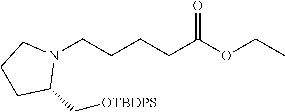

A mixture of ethyl 5-bromopentanoate (10 g, 47.83 mmol, 1 eq), tert-butyl-diphenyl-[[(2S)-pyrrolidin-2-yl]methoxy]silane (24.36 g, 71.74 mmol, 1.5 eq), potassium carbonate (19.83 g, 143.49 mmol, 3 eq) and potassium iodide (794 mg, 4.78 mmol, 0.1 eq) in N,N-dimethylformamide (100 mL) was stirred at 85° C. for 1 hour. The mixture was diluted with water (300 mL) and extracted with ethyl acetate (100 mL×3). The combined organic layers were washed with brine (100 mL×3), dried over anhydrous sodium sulfate, filtered and concentrated under reduced pressure to give a residue which was purified by column chromatography (silicon dioxide, Petroleum ether/Ethyl acetate=10/1 to 1/1) to afford the desired product (20 g, 42.76 mmol, 89% yield) as light yellow oil. LCMS: (ESI, m/z): 468.4 [M+1]⁺. ¹H NMR: (400 MHz, CDCl₃) δ: 7.76-7.63 (m, 4H), 7.49-7.30 (m, 6H), 4.19-4.07 (m, 2H), 3.80-3.60 (m, 1H), 3.59-3.39 (m, 1H), 3.20-2.99 (m, 1H), 2.88-2.48 (m, 2H), 2.39-2.09 (m, 4H), 1.87 (s, 1H), 1.79-1.68 (m, 2H), 1.65-1.51 (m, 4H), 1.50-1.44 (m, 1H), 1.28-1.22 (m, 3H), 1.06 (s, 9H).

Step 2: ethyl 5-[(2S)-2-(hydroxymethyl)pyrrolidin-1-yl] pentanoate

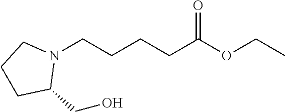 [SMILES: CCOC(=O)CCCCN1CCC[C@H]1CO]

To a solution of ethyl 5-[(2S)-2-[[tert-butyl(diphenyl)silyl]oxymethyl]pyrrolidin-1-yl] pentanoate (20 g, 42.76 mmol, 1 eq) in tetrahydrofuran (200 mL) was added tetrabutyl ammonium fluoride (1 M, 85.5 mL, 2 eq). The mixture was stirred at 25° C. for 12 hours. The reaction mixture was concentrated under reduced pressure to give a residue which was purified by column chromatography (silicon dioxide, Petroleum ether/Ethyl acetate=5/1 to 0/1) to afford the desired product (4.7 g, 20.50 mmol, 48% yield) as a light-yellow oil.

¹H NMR: (400 MHz, CDCl₃) δ: 4.19-4.08 (m, 2H), 3.74-3.62 (m, 1H), 3.47 (d, J=10.4 Hz, 1H), 3.27 (s, 1H), 2.90-2.61 (m, 2H), 2.47-2.26 (m, 4H), 2.02-1.49 (m, 9H), 1.27 (t, J=7.2 Hz, 3H).

Example 6: (2S,4R)-1-[(2S)-2-[3-[3-[(2S)-2-[[4-(3,8-diazabicyclo[3.2.1]octan-3-yl)-8-fluoro-7-(8-fluoro-3-hydroxy-1-naphthyl)pyrido[4,3-d]pyrimidin-2-yl]oxymethyl]pyrrolidin-1-yl]propoxy]propanoylamino]-3,3-dimethyl-butanoyl]-4-hydroxy-N-[(1S)-1-[4-(4-methylthiazol-5-yl)phenyl]ethyl]pyrrolidine-2-carboxamide

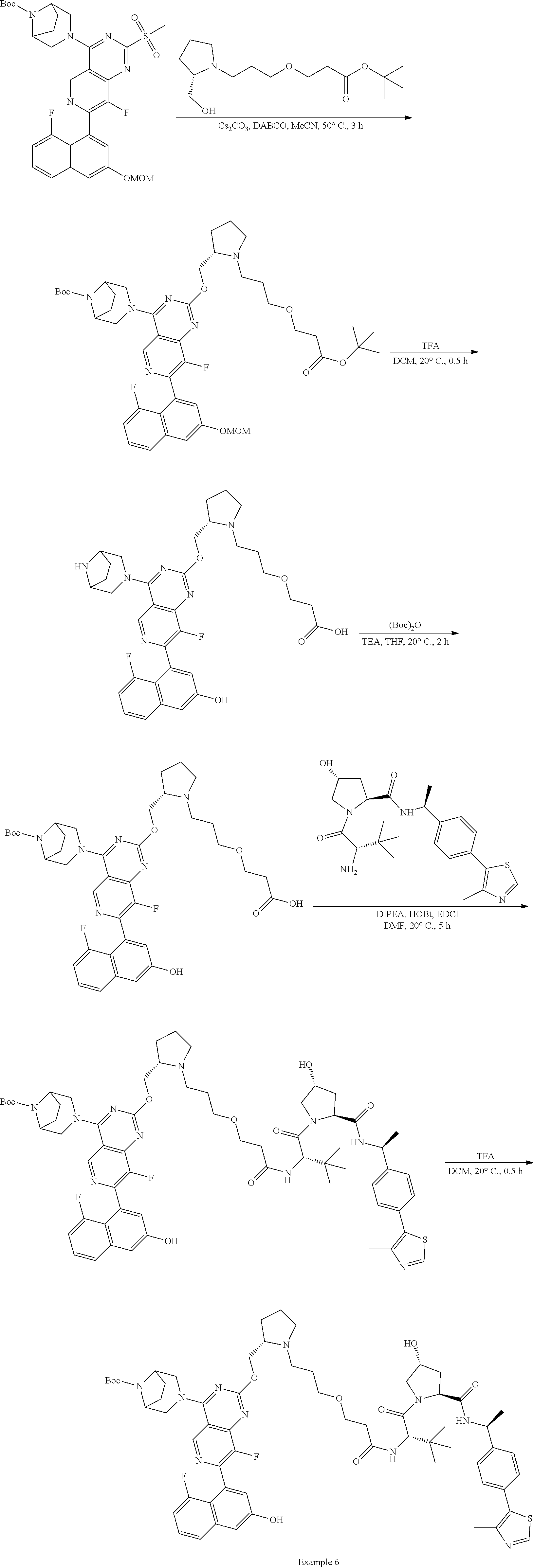

Step 1: tert-butyl 3-[2-[[(2S)-1-[3-(3-tert-butoxy-3-oxo-propoxy)propyl]pyrrolidin-2-yl]methoxy]-8-fluoro-7-[8-fluoro-3-(methoxymethoxy)-1-naphthyl]pyrido[4,3-d]pyrimidin-4-yl]-3,8-diazabicyclo[3.2.1]octane-8-carboxylate

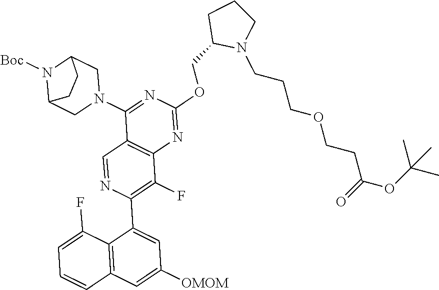

To a solution of tert-butyl 3-[8-fluoro-7-[8-fluoro-3-(methoxymethoxy)-1-naphthyl]-2-methylsulfonyl-pyrido[4,3-d]pyrimidin-4-yl]-3,8-diazabicyclo[3.2.1]octane-8-carboxylate (500 mg, 0.78 mmol, 1 eq) and tert-butyl 3-[3-[(2S)-2-(hydroxymethyl)pyrrolidin-1-yl]propoxy]propanoate (448 mg, 1.56 mmol, 2 eq) in acetonitrile (5 mL) was added cesium carbonate (508 mg, 1.56 mmol, 2 eq) and 1,4-diazabicyclo[2.2.2]octane (17 mg, 0.16 mmol, 0.2 eq). Then the mixture was stirred at 50° C. for 3 hours. The mixture was filtered and then the filtrate was concentrated in vacuum to generate a residue which was purified by prep-TLC (silica dioxide, dichloromethane/methanol=10/1) to afford the desired product (290 mg, 0.34 mmol, 44% yield) as a yellow oil. LCMS: (ESI, m/z): 849.5[M+1]⁺.

Step 2: 3-[3-[(2S)-2-[[4-(3,8-diazabicyclo[3.2.1] octan-3-yl)-8-fluoro-7-(8-fluoro-3-hydroxy-1-naphthyl)pyrido[4,3-d]pyrimidin-2-yl]oxymethyl]pyrrolidin-1-yl]propoxy]propanoic acid

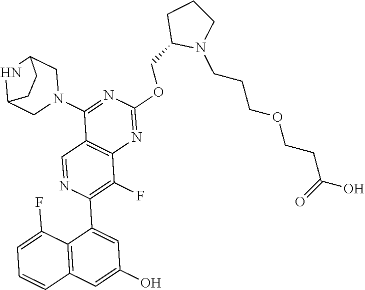 [SMILES: O=C(O)CCOCCCN1CCC[C@H]1COc1nc(N2CC3CCC(C2)N3)c2cnc(-c3cc(O)cc4cccc(F)c34)c(F)c2n1]

To a solution of tert-butyl 3-[2-[[(2S)-1-[3-(3-tert-butoxy-3-oxo-propoxy)propyl]pyrrolidin-2-yl]methoxy]-8-fluoro-7-[8-fluoro-3-(methoxymethoxy)-1-naphthyl]pyrido[4,3-d]pyrimidin-4-yl]-3,8-diazabicyclo[3.2.1]octane-8-carboxylate (100 mg, 0.12 mmol, 1 eq) in dichloromethane (1 mL) was added trifluoroacetic acid (0.3 mL). Then the mixture was stirred at 20° C. for 0.5 hour. The mixture was concentrated under reduced pressure to generate the crude product (76 mg, 0.12 mmol, 99% yield) as a yellow oil, which was used directly into the next step. LCMS: (ESI, m/z): 649.4[M+1]⁺.

Step 3: 3-[3-[(2S)-2-[[4-(8-tert-butoxycarbonyl-3,8-diazabicyclo[3.2.1]octan-3-yl)-8-fluoro-7-(8-fluoro-3-hydroxy-1-naphthyl)pyrido[4,3-d]pyrimidin-2-yl]oxymethyl]pyrrolidin-1-yl]propoxy]propanoic acid

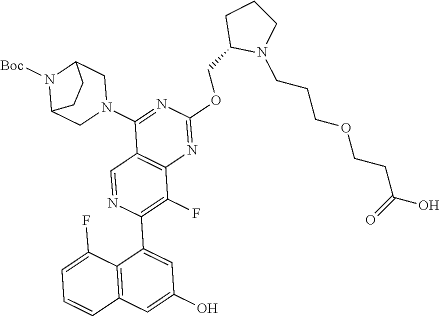 [SMILES: CC(C)(C)OC(=O)N1C2CCC1CN(c1nc(OC[C@@H]3CCCN3CCCOCCC(=O)O)nc3c(F)c(-c4cc(O)cc5cccc(F)c45)ncc13)C2]

To a solution of 3-[3-[(2S)-2-[[4-(3,8-diazabicyclo[3.2.1]octan-3-yl)-8-fluoro-7-(8-fluoro-3-hydroxy-1-naphthyl)pyrido[4,3-d]pyrimidin-2-yl]oxymethyl]pyrrolidin-1-yl]propoxy]propanoic acid (76 mg, 0.12 mmol, 1 eq) in tetrahydrofuran (2 mL) was added triethylamine (59 mg, 0.59 mmol, 5 eq) and di-tert-butyl dicarbonate (38 mg, 0.18 mmol, 1.5 eq). Then the mixture was stirred at 20° C. for 2 hours. The mixture was concentrated to generate a residue which was purified by prep-HPLC (column: Phenomenex Synergi C18 150*25 mm*10 um; mobile phase: [water(FA)-ACN]; B %: 16%-46%, 10 min) to afford the desired product (40 mg, 0.053 mmol, 45% yield) as a white solid. LCMS: (ESI, m/z): 749.8[M+1]⁺.

Step 4: tert-Butyl 3-[8-fluoro-7-(8-fluoro-3-hydroxy-1-naphthyl)-2-[[(2S)-1-[3-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]propyl]pyrrolidin-2-yl]methoxy]pyrido[4,3-d]pyrimidin-4-yl]-3,8-diazabicyclo[3.2.1]octane-8-carboxylate

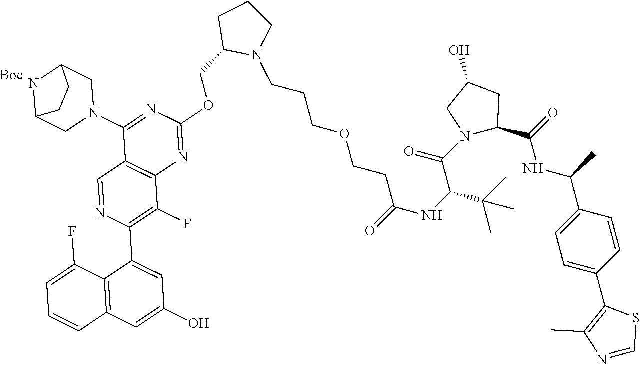 [SMILES: Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cnc(-c4cc(O)cc5cccc(F)c45)c(F)c3n2)C(C)(C)C)cc1 ; "Boc"]

To a solution of 3-[3-[(2S)-2-[[4-(8-tert-butoxycarbonyl-3,8-diazabicyclo[3.2.1]octan-3-yl)-8-fluoro-7-(8-fluoro-3-hydroxy-1-naphthyl)pyrido[4,3-d]pyrimidin-2-yl]oxymethyl]pyrrolidin-1-yl]propoxy]propanoic acid (40 mg, 0.053 mmol, 1 eq) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (38 mg, 0.080 mmol, 1.5 eq, hydrochloride) in N,N-dimethylformamide (1 mL) was added N,N-diisopropylethylamine (34 mg, 0.27 mmol, 5 eq) and 1-hydroxybenzotriazole (15 mg, 0.11 mmol, 2 eq). The mixture was stirred at 20° C. for 10 min. N-(3-dimethylaminopropyl)-N-ethylcarbodiimide hydrochloride (26 mg, 0.13 mmol, 2.5 eq) was added into the mixture and the stirring was continued at 20° C. for 5 hours. The mixture was diluted with water (10 mL), extracted with ethyl acetate (10 mL×3). The combined organic layers were washed with brine (10 mL), dried over anhydrous sodium sulfate, filtered and concentrated under reduced pressure to generate a residue which was purified by prep-TLC (silicon dioxide, dichloromethane/methanol=10/1) to afford the desired product (40 mg, 0.034 mmol, 63% yield) as a yellow oil. LCMS: (ESI, m/z): 1175.7[M]⁺.

Step 5: (2S,4R)-1-[(2S)-2-[3-[3-[(2S)-2-[[4-(3,8-diazabicyclo [3.2.1]octan-3-yl)-8-fluoro-7-(8-fluoro-3-hydroxy-1-naphthyl)pyrido[4,3-d]pyrimidin-2-yl]oxymethyl]pyrrolidin-1-yl]propoxy]propanoylamino]-3,3-dimethyl-butanoyl]-4-hydroxy-N-[(1S)-1-[4-(4-methylthiazol-5-yl)phenyl]ethyl]pyrrolidine-2-carboxamide

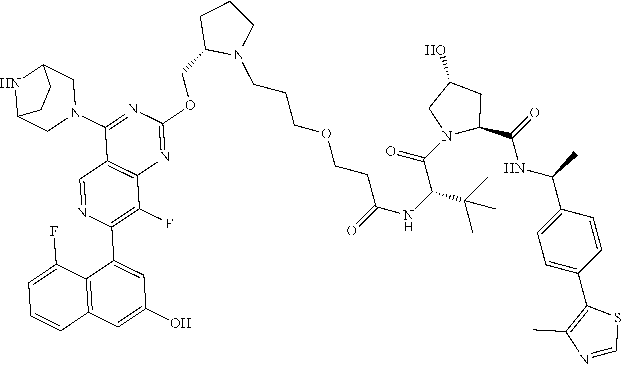 [SMILES: Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cnc(-c4cc(O)cc5cccc(F)c45)c(F)c3n2)C(C)(C)C)cc1]

To a solution of tert-butyl 3-[8-fluoro-7-(8-fluoro-3-hydroxy-1-naphthyl)-2-[[(2S)-1-[3-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]propyl]pyrrolidin-2-yl]methoxy]pyrido[4,3-d]pyrimidin-4-yl]-3,8-diazabicyclo[3.2.1]octane-8-carboxylate (40 mg, 0.034 umol, 1 eq) in dichloromethane (3 mL) was added trifluoroacetic acid (0.5 mL). Then the mixture was stirred at 25° C. for 0.5 h. The mixture was concentrated under reduced pressure to generate a residue which was purified by prep-HPLC (column: Phenomenex Synergi C18 150*25 mm*10 um; mobile phase: [water(FA)-ACN]; B %: 13%-33%, 10 min) to afford the desired product (17.67 mg, 0.016 mmol, 45% yield, 98% purity, formate[3]) as a yellow solid. LCMS: (ESI, m/z): 1075.4 [M]⁺. ¹H NMR: (400 MHz, DMSO-d₆) δ: 9.10 (s, 1H), 8.98 (s, 1H), 8.38 (d, J=8.0 Hz, 1H), 8.23 (br s, 3H), 7.82 (d, J=9.6 Hz, 1H), 7.66 (d, J=8.0 Hz, 1H), 7.47-7.39 (m, 3H), 7.37 (d, J=8.0 Hz, 3H), 7.16 (d, J=2.4 Hz, 1H), 7.04-6.96 (m, 1H), 4.94-4.86 (m, 1H), 4.53-4.40 (m, 4H), 4.39-4.35 (m, 1H), 4.26 (s, 1H), 4.13-4.07 (m, 1H), 3.65 (s, 8H), 3.08-3.02 (m, 2H), 2.94-2.77 (m, 4H), 2.46-2.44 (m, 3H), 2.40-2.34 (m, 1H), 2.30-2.14 (m, 3H), 2.04-1.97 (m, 1H), 1.95-1.88 (m, 1H), 1.69 (d, J=7.2 Hz, 11H), 1.36 (d, J=7.2 Hz, 3H), 0.89 (s, 9H).

Example 7: (2S,4R)-1-[(2S)-2-[5-[(2S)-2-[[7-(2-amino-7-fluoro-1,3-benzothiazol-4-yl)-6-chloro-4-(3,8-diazabicyclo[3.2.1]octan-3-yl)-8-fluoro-quinazolin-2-yl]oxymethyl]pyrrolidin-1-yl]pentanoylamino]-3,3-dimethyl-butanoyl]-4-hydroxy-N-[(1S)-1-[4-(4-methylthiazol-5-yl)phenyl]ethyl]pyrrolidine-2-carboxamide

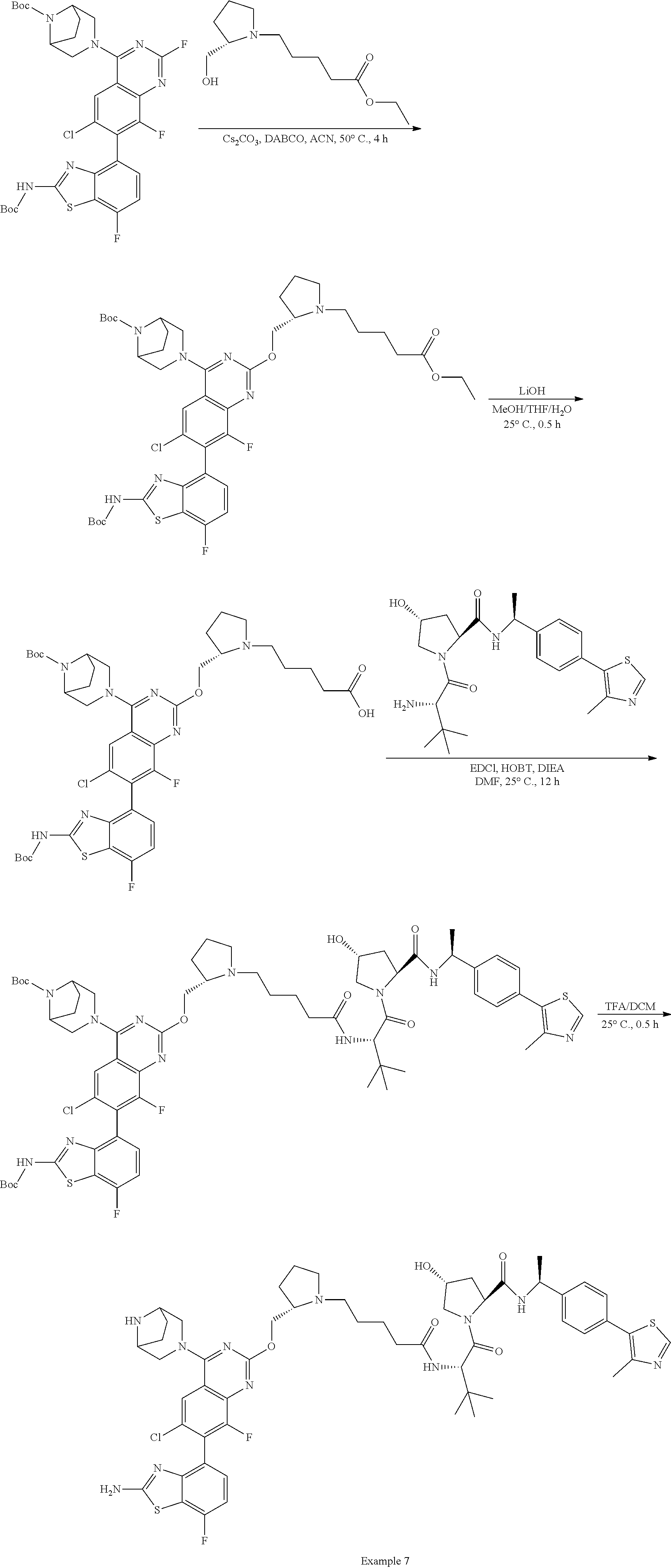

Step 1: tert-butyl3-[7-[2-(tert-butoxycarbonylamino)-7-fluoro-1,3-benzothiazol-4-yl]-6-chloro-2-[[(2S)-1-(5-ethoxy-5-oxo-pentyl)pyrrolidin-2-yl]methoxy]-8-fluoro-quinazolin-4-yl]-3,8-diazabicyclo[3.2.1]octane-8-carboxylate

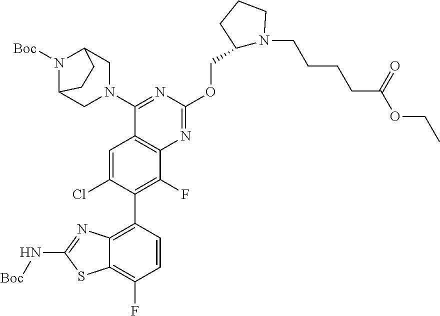 [SMILES: CCOC(=O)CCCCN1CCC[C@H]1COc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of tert-butyl 3-[7-[2-(tert-butoxycarbonylamino)-7-fluoro-1,3-benzothiazol-4-yl]-6-chloro-2,8-difluoro-quinazolin-4-yl]-3,8-diazabicyclo[3.2.1]octane-8-carboxylate (200 mg, 0.29 mmol, 1 eq) and ethyl 5-[(2S)-2-(hydroxymethyl)pyrrolidin-1-yl]pentanoate (203 mg, 0.88 mmol, 3 eq) in acetonitrile (5 mL) was added cesium carbonate (289 mg, 0.88 mmol, 3 eq) and 1,4-diazabicyclo[2.2.2]octane (3 mg, 0.02 mmol, 0.1 eq). The mixture was stirred at 50° C. for 4 hours. The reaction mixture was concentrated under reduced pressure to remove the solvent to get a residue which was purified by prep-TLC (9% methanol in dichloromethane) to afford the desired product (85.6 mg, 0.09 mmol, 32% yield) as a white solid. LCMS: (ESI, m/z): 886.2 [M]⁺.

Step 2: 5-[(2S)-2-[[7-[2-(tert-butoxycarbonylamino)-7-fluoro-1,3-benzothiazol-4-yl]-4-(8-tert-butoxycarbonyl-3,8-diazabicyclo[3.2.1]octan-3-yl)-6-chloro-8-fluoro-quinazolin-2-yl]oxymethyl]pyrrolidin-1-yl]pentanoic acid

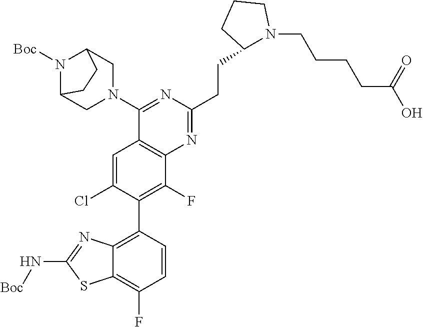 [SMILES: CC(C)(C)OC(=O)Nc1nc2c(-c3c(Cl)cc4c(N5CC6CCC(C5)N6C(=O)OC(C)(C)C)nc(CC[C@@H]5CCCN5CCCCC(=O)O)nc4c3F)ccc(F)c2s1]

To a solution of tert-butyl 3-[7-[2-(tert-butoxycarbonylamino)-7-fluoro-1,3-benzothiazol-4-yl]-6-chloro-2-[[(2S)-1-(5-ethoxy-5-oxo-pentyl)pyrrolidin-2-yl]methoxy]-8-fluoro-quinazolin-4-yl]-3,8-diazabicyclo[3.2.1]octane-8-carboxylate (107 mg, 0.12 mmol, 1 eq) in the mixed solvent of methanol (1 mL), tetrahydrofuran (1 mL) and water (1 mL) was added lithium hydroxide monohydrate (51 mg, 1.21 mmol, 10 eq). The mixture was stirred at 25° C. for 0.5 hour. The mixture was filtered and concentrated under vacuum to give a residue which was re-dissolved by water. Hydrochloride (0.5M) was added to adjust the pH to be 5. The aqueous phase was extracted with ethyl acetate (10 mL×3). The combined organic layers were washed with brine (10 mL×2), dried with anhydrous sodium sulfate, filtered and concentrated in vacuum to get a residue which was purified by prep-TLC (11% methanol in dichloromethane) to afford the desired product (74 mg, 0.08 mmol, 71% yield) as a white solid. LCMS: (ESI, m/z): 858.2 [M]⁺.

Step 3: tert-butyl3-[7-[2-(tert-butoxycarbonylamino)-7-fluoro-1,3-benzothiazol-4-yl]-6-chloro-8-fluoro-2-[[(2S)-1-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-5-oxo-pentyl]pyrrolidin-2-yl]methoxy]quinazolin-4-yl]-3,8-diazabicyclo[3.2.1]octane-8-carboxylate

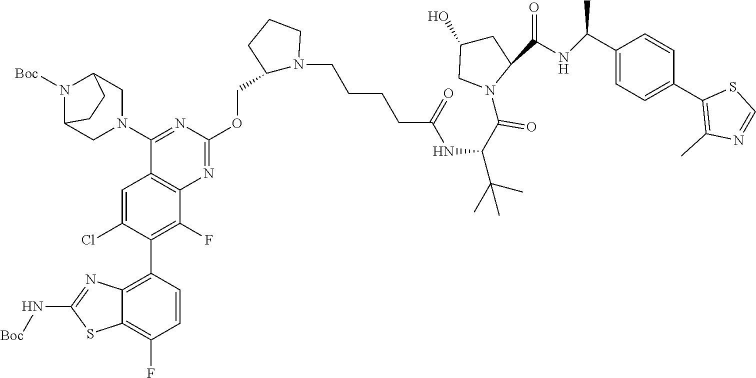 [SMILES: Cc1ncsc1-c1ccc([C@H](C)NC(=O)[C@@H]2C[C@@H](O)CN2C(=O)[C@@H](NC(=O)CCCCN2CCC[C@H]2COc2nc(N3CC4CCC(C3)N4C(=O)OC(C)(C)C)c3cc(Cl)c(-c4ccc(F)c5sc(NC(=O)OC(C)(C)C)nc45)c(F)c3n2)C(C)(C)C)cc1]

To a solution of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (21 mg, 0.04 mmol, 1 eq, hydrochloride) in N,N-dimethylformamide (2 mL) was added N,N-diisopropylethylamine (28 mg, 0.21 mmol, 5 eq), then 5-[(2S)-2-[[7-[2-(tert-butoxycarbonylamino)-7-fluoro-1,3-benzothiazol-4-yl]-4-(8-tert-butoxycarbonyl-3,8-diazabicyclo [3.2.1]octan-3-yl)-6-chloro-8-fluoro-quinazolin-2-yl]oxymethyl]pyrrolidin-1-yl]pentanoic acid (37 mg, 0.04 mmol, 1 eq), 1-hydroxybenzotriazole (17 mg, 0.12 mmol, 3 eq) and 1-(3-dimethylaminopropyl)-3-ethylcarbodiimide(25 mg, 0.12 mmol, 3 eq) were added. The reaction mixture was stirred at 25° C. for 12 hours. The aqueous phase was extracted with ethyl acetate (10 mL×3). The combined organic layers were washed with brine (10 mL×2), dried with anhydrous sodium sulfate, filtered and concentrated in vacuum to get a residue which was purified by prep-TLC (9% methanol in dichloromethane) to afford the desired product (20 mg, 0.01 mmol, 36% yield) as a white solid. LCMS: (ESI, m/z): 1284.2 [M]⁺.

Step 4: (2S,4R)-1-[(2S)-2-[5-[(2S)-2-[[7-(2-amino-7-fluoro-1,3-benzothiazol-4-yl)-6-chloro-4-(3,8-diazabicyclo[3.2.1]octan-3-yl)-8-fluoro-quinazolin-2-yl]oxymethyl]pyrrolidin-1-yl]pentanoylamino]-3,3-dimethyl-butanoyl]-4-hydroxy-N-[(1S)-1-[4-(4-methylthiazol-5-yl)phenyl]ethyl]pyrrolidine-2-carboxamide

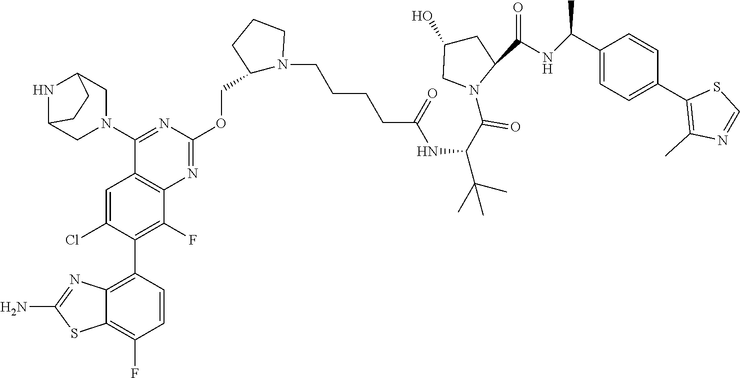 [SMILES: Cc1ncsc1-c1ccc([C@H](C)NC(=O)[C@@H]2C[C@@H](O)CN2C(=O)[C@@H](NC(=O)CCCCN2CCC[C@H]2COc2nc(N3CC4CCC(C3)N4)c3cc(Cl)c(-c4ccc(F)c5sc(N)nc45)c(F)c3n2)C(C)(C)C)cc1]

To a solution of tert-butyl 3-[7-[2-(tert-butoxycarbonylamino)-7-fluoro-1,3-benzothiazol-4-yl]-6-chloro-8-fluoro-2-[[(2S)-1-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-5-oxo-pentyl]pyrrolidin-2-yl]methoxy]quinazolin-4-yl]-3,8-diazabicyclo[3.2.1]octane-8-carboxylate (20 mg, 0.01 mmol, 1 eq) in dichloromethane (1 mL) was added trifluoroacetic acid (1.54 g, 1 mL, 867.74 eq). The mixture was stirred at 25° C. for 0.5 hour. The reaction mixture was concentrated under reduced pressure to give a residue which was purified by prep-HPLC (column: Phenomenex Synergi C18 150*25 mm*10 um; mobile phase: [water(FA)-ACN]; B %: 13%-43%, 10 min) to afford the desired product (14.5 mg, 0.01 mmol, 82% yield, 99% purity, formate[1]) as a white solid. LCMS: (ESI, m/z): 1084.3[M]⁺. ¹H NMR: EW30163-28-P1, (400 MHz, DMSO-d₆) δ: 8.98 (s, 1H), 8.37 (d, J=7.2 Hz, 1H), 8.24 (s, 1H), 8.30 (s, 2H), 7.90-7.98 (m, 1H), 7.83-7.88 (m, 1H), 7.77 (br d, J=9.0 Hz, 2H), 7.35-7.50 (m, 2H), 7.25-7.20 (m, 1H), 7.01-7.11 (m, 1H), 4.81-4.98 (m, 1H), 4.47-4.55 (m, 1H), 4.38-4.46 (m, 2H), 4.21-4.34 (m, 5H), 3.98-4.11 (m, 3H), 3.59-3.68 (m, 8H), 2.54 (s, 2H), 2.73-3.18 (m, 3H), 2.06-2.22 (m, 2H), 1.96-2.05 (m, 1H), 1.85-1.95 (m, 1H), 1.75-1.84 (m, 1H), 1.55-1.74 (m, 8H), 1.41-1.48 (m, 2H), 1.33-1.40 (m, 3H), 0.91 (s, 9H).

Example 8: (2S,4R)-1-[(2S)-2-[3-[3-[2-[[7-(2-amino-3-cyano-7-fluoro-benzothiophen-4-yl)-6-chloro-4-(3,8-diazabicyclo[3.2.1]octan-3-yl)-8-fluoro-quinazolin-2-yl]oxymethyl]pyrrolidin-1-yl]propoxy]propanoylamino]-3,3-dimethyl-butanoyl]-4-hydroxy-N-[(1S)-1-[4-(4-methylthiazol-5-yl)phenyl]ethyl]pyrrolidine-2-carboxamide

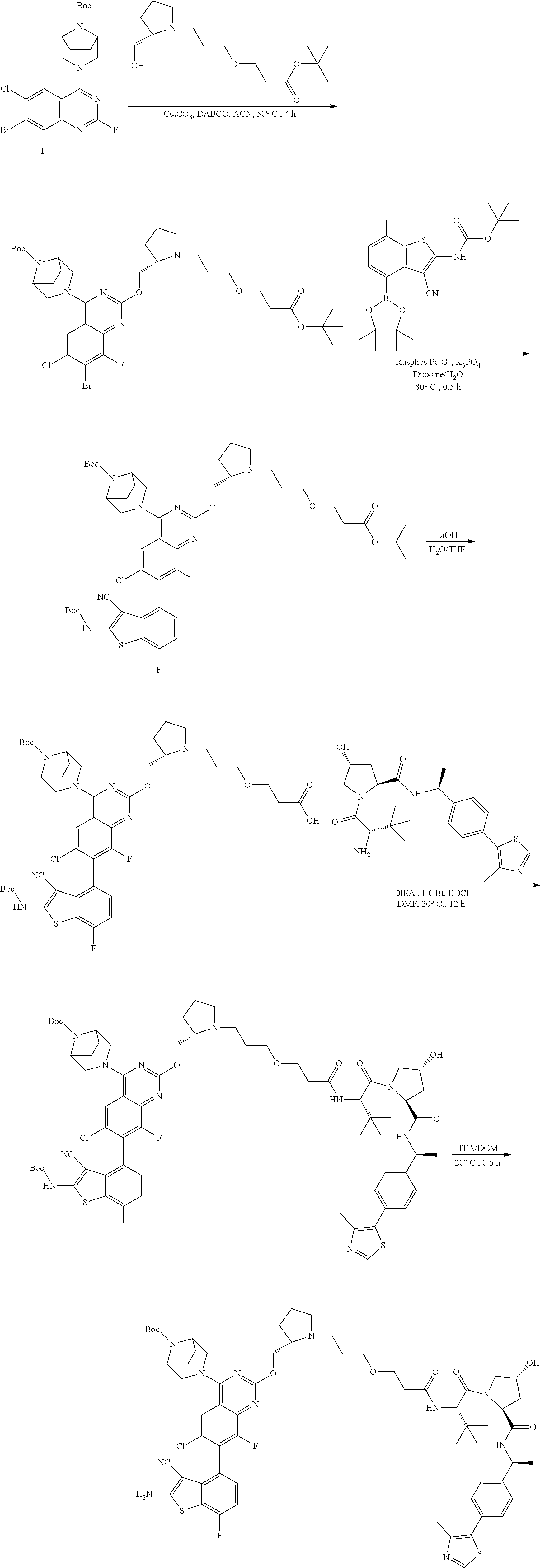

Step 1: tert-butyl 3-[7-bromo-2-[[1-[3-(3-tert-butoxy-3-oxo-propoxy)propyl]pyrrolidin-2-yl]methoxy]-6-chloro-8-fluoro-quinazolin-4-yl]-3,8-diazabicyclo[3.2.1]octane-8-carboxylate

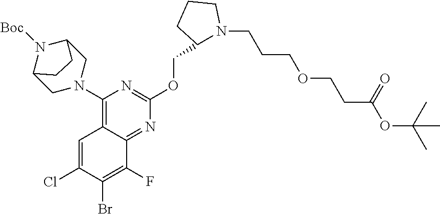

To a solution of tert-butyl 3-(7-bromo-6-chloro-2,8-difluoro-quinazolin-4-yl)-3,8-diazabicyclo[3.2.1]octane-8-carboxylate (1 g, 2.04 mmol, 1 eq) and tert-butyl 3-[3-[(2S)-2-(hydroxymethyl)pyrrolidin-1-yl]propoxy]propanoate (1.17 g, 4.08 mmol, 2 eq) in acetonitrile (10 mL) was added cesium carbonate (1.33 g, 4.08 mmol, 2 eq) and 1,4-diazabicyclo[2.2.2]octane (22 mg, 0.20 mmol, 0.1 eq). The mixture was stirred at 40° C. for 3 h. Water (20 ml) was added before the mixture was extracted by ethyl acetate (20 mL×3). The combined organic layers were washed with brine (20 mL×3), dried over anhydrous sodium sulfate, filtered and concentrated under reduced pressure to give a residue which was purified by prep-HPLC (column: Phenomenex Synergi Max-RP 250*50 mm*10 um; mobile phase: [water(FA)-ACN]; B %: 35%-65%, 21 min) to give the desired product (529 mg, 0.63 mmol, 31% yield, 91% purity) as a yellow oil. LCMS: (ESI, m/z): 757.9 [M+1]⁺. ¹H NMR: (400 MHz, CDCl₃) δ: 8.42-8.39 (m, 1H), 4.74 (d, J=4.8, 11.2 Hz, 1H), 4.39 (s, 2H), 4.37-4.31 (m, 4H), 3.65 (t, J=6.4 Hz, 4H), 3.56-3.48 (m, 6H), 3.33-3.21 (m, 6H), 2.79 (d, J=3.2 Hz, 1H), 2.62 (d, J=9.6 Hz, 1H), 2.20-2.10 (m, 2H), 2.02-1.92 (m, 9H), 1.77 (d, J=3.2 Hz, 2H), 1.44 (s, 9H).

Step 2: tert-butyl 3-[7-[2-(tert-butoxycarbonylamino)-3-cyano-7-fluoro-benzothiophen-4-yl]-2-[[1-[3-(3-tert-butoxy-3-oxo-propoxy)propyl]pyrrolidin-2-yl]methoxy]-6-chloro-8-fluoro-quinazolin-4-yl]-3,8-diazabicyclo[3.2.1]octane-8-carboxylate

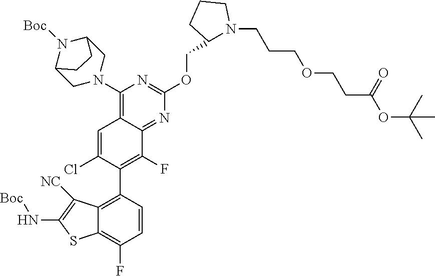

A mixture of tert-butyl 3-[7-bromo-2-[[1-[3-(3-tert-butoxy-3-oxo-propoxy)propyl]pyrrolidin-2-yl]methoxy]-6-chloro-8-fluoro-quinazolin-4-yl]-3,8-diazabicyclo[3.2.1]octane-8-carboxyl-ate (879 mg, 1.16 mmol, 1 eq), tert-butyl N-[3-cyano-7-fluoro-4-(4,4,5,5-tetramethyl-1,3,2-dioxaborolan-2-yl)benzothiophen-2-yl]carbamate (728 mg, 1.74 mmol, 1.5 eq), sodium carbonate (369 mg, 3.48 mmol, 3 eq) in dioxane (5 mL) and water (1 mL) was degassed and purged with nitrogen for 3 times. Then ditert-butyl(cyclopentyl)phosphane; dichloropalladium; iron (75 mg, 0.12 mmol, 0.1 eq) was added, and the mixture was stirred at 90° C. for 2 h in nitrogen atmosphere. Water (20 mL) was added before the mixture was extracted by ethyl acetate (20 mL×3). The combined organic layers were washed with brine (20 mL×3), dried over anhydrous sodium sulfate, filtered and concentrated under reduced pressure to give a residue which was purified by prep-HPLC (column: Phenomenex luna C18 250*50 mm*15 um; mobile phase: [water (FA)-ACN]; B %: 47%-77%, 10 min) to give the desired product (113 mg, 0.11 mmol, 9% yield, 94% purity) as a yellow solid. ¹H NMR: (400 MHz, CDCl₃) δ: 7.74 (s, 1H), 7.31 (d, J=4.4, 8.4 Hz, 1H), 7.16 (t, J=8.4 Hz, 1H), 4.55-4.27 (m, 5H), 4.26-4.06 (m, 3H), 3.71-3.55 (m, 4H), 3.55-3.43 (m, 2H), 3.21-3.09 (m, 1H), 3.02-2.86 (m, 1H), 2.45 (t, J=5.6 Hz, 2H), 2.05 (s, 3H), 2.00-1.91 (m, 3H), 1.90-1.72 (m, 6H), 1.57 (s, 9H), 1.53 (s, 9H), 1.43 (s, 9H).

Step 3: 3-[3-[2-[[7-[2-(tert-butoxycarbonylamino)-3-cyano-7-fluoro-benzothiophen-4-yl]-4-(8-tert-butoxycarbonyl-3,8-diazabicyclo[3.2.1]octan-3-yl)-6-chloro-8-fluoro-quinazolin-2-yl]oxymethyl]pyrrolidin-1-yl]propoxy]propanoic acid

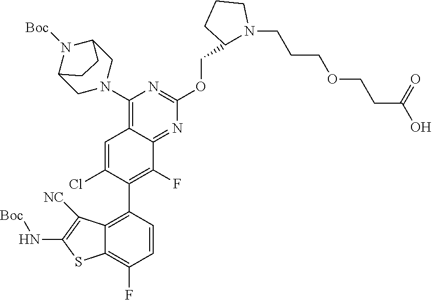

To a solution of tert-butyl 3-[7-[2-(tert-butoxycarbonylamino)-3-cyano-7-fluoro-benzothiophen-4-yl]-2-[[1-[3-(3-tert-butoxy-3-oxo-propoxy)propyl]pyrrolidin-2-yl]methoxy]-6-chloro-8-fluoro-quinazolin-4-yl]-3,8-diazabicyclo[3.2.1]octane-8-carboxylate (100 mg, 0.10 mmol, 1 eq) in water (0.2 mL), methanol (0.6 mL) and tetrahydrofuran (0.6 mL) was added lithium hydroxide (24 mg, 1.03 mmol, 10 eq). The mixture was stirred at 25° C. for 12 h. The reaction mixture was concentrated under reduced pressure to remove solvent. The resulting mixture was dissolved in water (10 mL) and adjusted to pH=6 by using aqueous hydrochloric acid (1 M). The mixture was extracted with ethyl acetate (20 mL×3), filtered and concentrated under reduced pressure to give a residue which was purified by prep-TLC (silicon dioxide, dichloromethane: methanol=10:1) to afford the desired product (74 mg, 0.08 mmol, 75% yield, 95% purity) as a yellow solid. LCMS: (ESI, m/z): 912.3[M]⁺.

Step 4: tert-butyl 3-[7-[2-(tert-butoxycarbonylamino)-3-cyano-7-fluoro-benzothiophen-4-yl]-6-chloro-8-fluoro-2-[[1-[3-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]propyl]pyrrolidin-2-yl]methoxy]quinazolin-4-yl]-3,8-diazabicyclo[3.2.1]octane-8-carboxylate

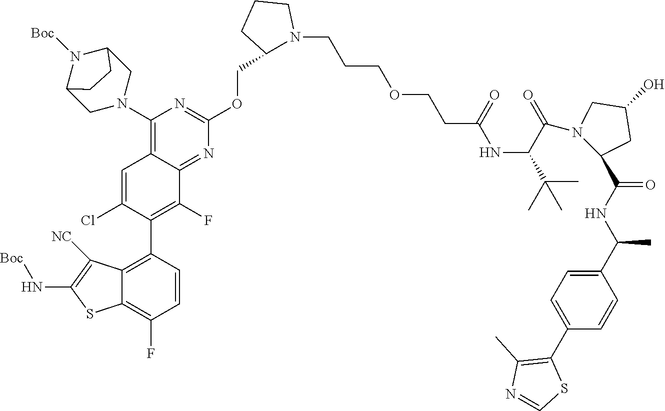 [SMILES: Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4C(=O)OC(C)(C)C)c3cc(Cl)c(-c4ccc(F)c5sc(NC(=O)OC(C)(C)C)c(C#N)c45)c(F)c3n2)C(C)(C)C)cc1]

To a solution of 3-[3-[2-[[7-[2-(tert-butoxycarbonylamino)-3-cyano-7-fluoro-benzothiophen-4-yl]-4-(8-tert-butoxycarbonyl-3,8-diazabicyclo[3.2.1]octan-3-yl)-6-chloro-8-fluoro-quinazolin-2-yl]oxymethyl]pyrrolidin-1-yl]propoxy]propanoic acid (74 mg, 0.08 mmol, 1 eq) and (2S,4R)-1-[(2S)-2-amino-3,3-dimethyl-butanoyl]-4-hydroxy-N-[(1S)-1-[4-(4-methylthiazol-5-yl)phen-yl]ethyl]pyrrolidine-2-carboxamide (43 mg, 0.10 mmol, 1.2 eq) in N,N-dimethylformamide (4 mL) was added N,N-diisopropylethylamine (52 mg, 0.4 mmol, 5 eq) and 1-hydroxybenzotriazole (21 mg, 0.16 mmol, 2 eq). The mixture was stirred at 20° C. for 15 min before N-(3-dimethylaminopropyl)-N-ethylcarbodiimide hydrochloride (38 mg, 0.20 mmol, 2.5 eq) was added. The mixture was stirred at 20° C. for 12 h. Water (10 ml) was added before the mixture was extracted by ethyl acetate (10 mL×3). The combined organic layers were washed with brine (10 mL×3), dried over anhydrous sodium sulfate, filtered and concentrated under reduced pressure to give a residue which was purified by prep-TLC (silicon dioxide, dichloromethane: methanol=10:1) to generate the desired product (91 mg, 0.06 mmol, 70% yield, 83% purity) as a yellow solid. LCMS: (ESI, m/z): 1338.4 [M]⁺.

Step 5: (2S,4R)-1-[(2S)-2-[3-[3-[2-[[7-(2-amino-3-cyano-7-fluoro-benzothiophen-4-yl)-6-chloro-4-(3,8-diazabicyclo[3.2.1]octan-3-yl)-8-fluoro-quinazolin-2-yl]oxymethyl]pyrrolidin-1-yl]propoxy]propanoylamino]-3,3-dimethyl-butanoyl]-4-hydroxy-N-[(1S)-1-[4-(4-methylthiazol-5-yl)phenyl]ethyl]pyrrolidine-2-carboxamide (Example 8)

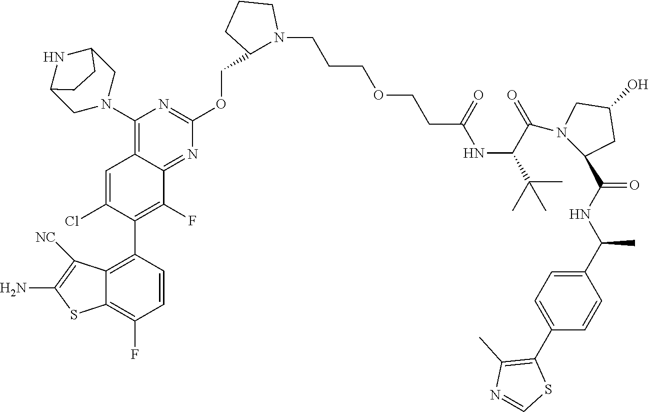

To a solution of tert-butyl 3-[7-[2-(tert-butoxycarbonylamino)-3-cyano-7-fluoro-benzothiophen-4-yl]-6-chloro-8-fluoro-2-[[1-[3-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-prop-yl]amino]-3-oxo-propoxy]propyl]pyrrolidin-2-yl]methoxy]quinazolin-4-yl]-3,8-diazabicyclo [3.2.1]octane-8-carboxylate (91 mg, 0.07 mmol, 1 eq) in dichloromethane (4 mL) was added trifluoroacetic acid (581 mg, 5.10 mmol, 75 eq). The mixture was stirred at 20° C. for 0.5 h. The reaction mixture was bubbled by nitrogen gas to remove dichloromethane. The residue was purified by prep-HPLC (column: Phenomenex Synergi C18 150*25 mm*10 um; mobile phase: [water (FA)-ACN]; B %: 10%-40%, 10 min) to give the desired product (42.04 mg, 0.03 mmol, 51% yield, 98% purity, formate[2]) as a white solid. LCMS: (ESI, m/z): 1138.4[M]⁺. ¹H NMR: (400 MHz, DMSO-d₆) δ: 8.99 (s, 1H), 8.38 (d, J=8.0 Hz, 1H), 8.24 (s, 2H), 8.11 (s, 2H), 7.90-7.75 (m, 2H), 7.48-7.33 (m, 4H), 7.27 (d, J=5.2 Hz, 1H), 7.20-7.10 (m, 1H), 4.94-4.88 (m, 1H), 4.52 (d, J=9.6 Hz, 1H), 4.44-4.40 (m, 1H), 4.38-4.31 (m, 2H), 4.31-4.24 (m, 3H), 4.06 (d, J=7.2 Hz, 2H), 3.59 (s, 5H), 3.07-3.02 (m, 2H), 2.92-2.77 (m, 5H), 2.46 (s, 3H), 2.38 (d, J=4.8, 7.6 Hz, 1H), 2.31-2.26 (m, 1H), 2.18 (d, J=7.6 Hz, 1H), 2.05-1.99 (m, 1H), 1.94-1.88 (m, 1H), 1.80 (d, J=4.4 Hz, 1H), 1.74-1.57 (m, 10H), 1.37 (d, J=6.8 Hz, 3H), 0.90 (s, 9H).

Example 9: (2S,4R)-1-[(2S)-2-[3-[3-[(2S)-2-[[7-(2-amino-3-cyano-7-fluoro-benzothiophen-4-yl)-6-chloro-4-(3,8-diazabicyclo[3.2.1]octan-3-yl)-8-fluoro-quinazolin-2-yl]oxymethyl]pyrrolidin-1-yl]propoxy]propanoylamino]-3,3-dimethyl-butanoyl]-4-hydroxy-N-[(1R)-2-hydroxy-1-[4-(4-methylthiazol-5-yl)phenyl]ethyl]pyrrolidine-2-carboxamide

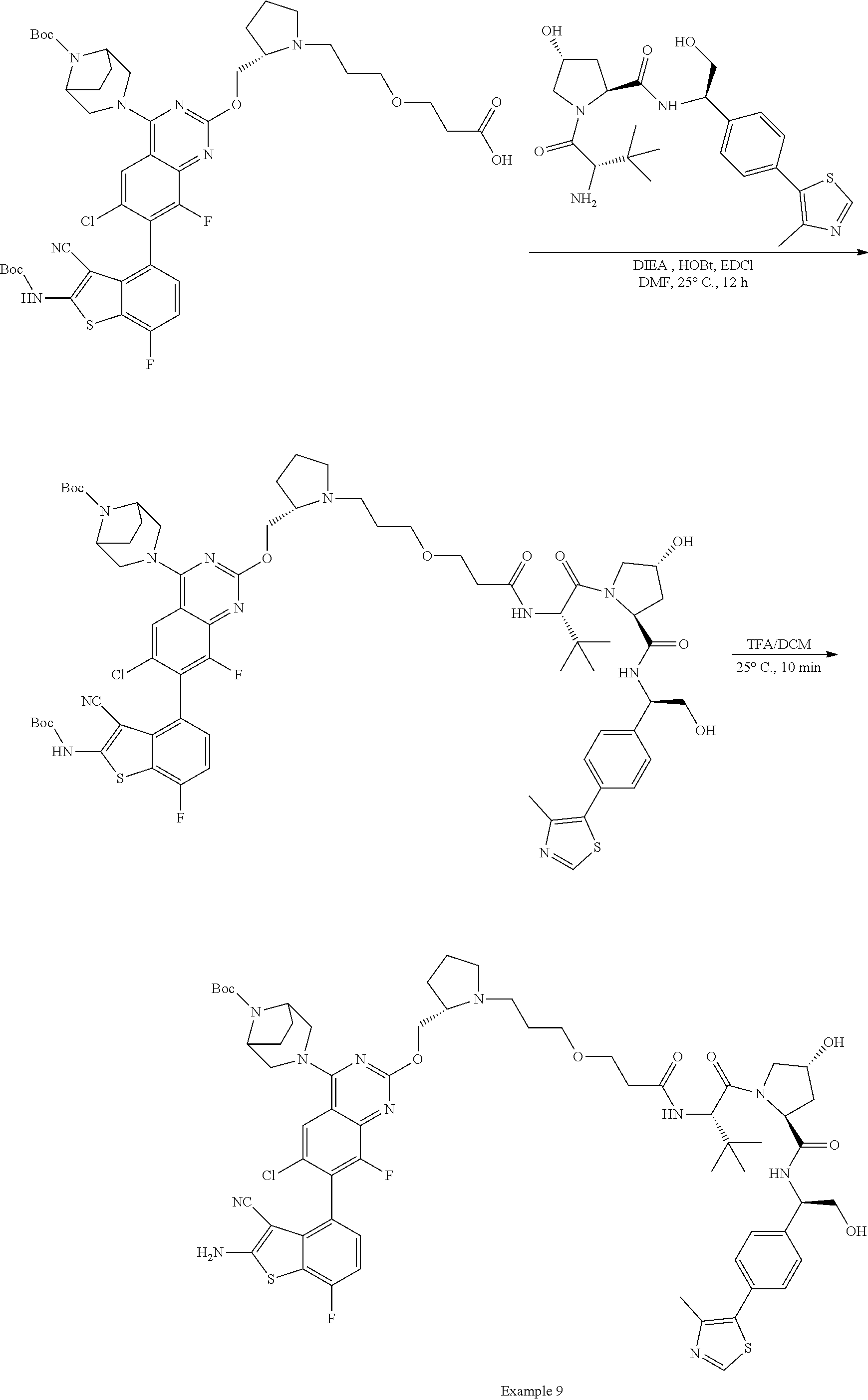

Step 1: tert-butyl 3-[7-[2-(tert-butoxycarbonylamino)-3-cyano-7-fluoro-benzothiophen-4-yl]-6-chloro-8-fluoro-2-[[(2S)-1-[3-[3-[[(1S)-1-[(2S,4R)-4-hydroxy-2-[[(1R)-2-hydroxy-1-[4-(4-methylthiazol-5-yl)phenyl]ethyl]carbamoyl]pyrrolidine-1-carbonyl]-2,2-dimethyl-propyl]amino]-3-oxo-propoxy]propyl]pyrrolidin-2-yl]methoxy]quinazolin-4-yl]-3,8-diazabicyclo[3.2.1]octane-8-carboxylate

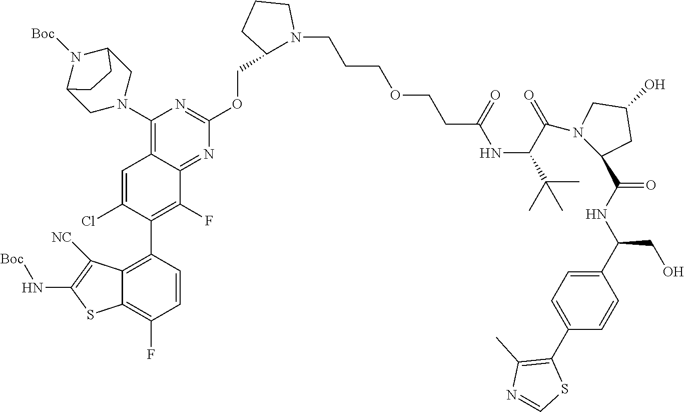 [SMILES: Cc1ncsc1-c1ccc([C@H](CO)NC(=O)[C@@H]2C[C@@H](O)CN2C(=O)[C@@H](NC(=O)CCOCCCN2CCC[C@H]2COc2nc(N3CC4CCC(C3)N4C(=O)OC(C)(C)C)c3cc(Cl)c(-c4ccc(F)c5sc(NC(=O)OC(C)(C)C)c(C#N)c45)c(F)c3n2)C(C)(C)C)cc1]

To a solution of (2S,4R)-1-[(2S)-2-amino-3,3-dimethyl-butanoyl]-4-hydroxy-N-[(1R)-2-hydroxy-1-[4-(4-methylthiazol-5-yl)phenyl]ethyl]pyrrolidine-2-carboxamide (25 mg, 0.05 mmol, 0.9 eq, hydrochloride) in N,N-dimethylformamide (2 mL) was added diisopropylethylamine (35 mg, 0.27 mmol, 5 eq), then 3-[3-[2-[[7-[2-(tert-butoxycarbonylamino)-3-cyano-7-fluoro-benzothiophen-4-yl]-4-(8-tert-butoxycarbonyl-3,8-diazabicyclo[3.2.1]octan-3-yl)-6-chloro-8-fluoro-quinazolin-2-yl]oxymethyl]pyrrolidin-1-yl]propoxy]propanoic acid (50 mg, 0.05 mmol, 1 eq), 1-hydroxybenzotriazole (22 mg, 0.16 mmol, 3 eq) and 1-(3-dimethylaminopropyl)-3-ethylcarbodiimide hydrochloride (31 mg, 0.16 mmol, 3 eq) were added. The mixture was stirred at 25° C. for 12 h. LCMS showed the reaction was completed. The reaction mixture was quenched by formic acid (1 mL), then concentrated in vacuum to give a residue which was purified by prep-HPLC (column: Phenomenex luna C18 150*40 mm*15 um; mobile phase: [water(FA)-ACN]; B %: 35%-65%, 10 min) to afford the desired product (45 mg, 0.03 mmol, 60% yield) as a yellow solid. LCMS: (ESI, m/z): 1357.1[M+1]⁺.

Step 2: (2S,4R)-1-[(2S)-2-[3-[3-[(2S)-2-[[7-(2-amino-3-cyano-7-fluoro-benzothiophen-4-yl)-6-chloro-4-(3,8-diazabicyclo[3.2.1]octan-3-yl)-8-fluoro-quinazolin-2-yl]oxymethyl]pyrrolidin-1-yl]propoxy]propanoylamino]-3,3-dimethyl-butanoyl]-4-hydroxy-N-[(1R)-2-hydroxy-1-[4-(4-methylthiazol-5-yl)phenyl]ethyl]pyrrolidine-2-carboxamide (Example 9)

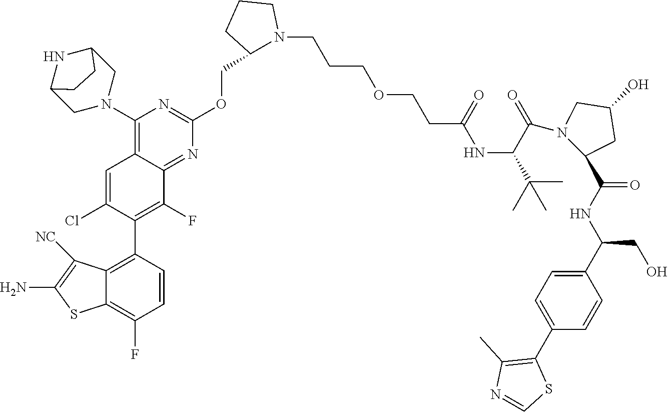 [SMILES: Cc1ncsc1-c1ccc([C@H](CO)NC(=O)[C@@H]2C[C@@H](O)CN2C(=O)[C@@H](NC(=O)CCOCCCN2CCC[C@H]2COc2nc(N3CC4CCC(C3)N4)c3cc(Cl)c(-c4ccc(F)c5sc(N)c(C#N)c45)c(F)c3n2)C(C)(C)C)cc1]

To a solution of tert-butyl3-[7-[2-(tert-butoxycarbonylamino)-3-cyano-7-fluoro-benzoth iophen-4-yl]-6-chloro-8-fluoro-2-[[(2S)-1-[3-[3-[[(1S)-1-[(2S,4R)-4-hydroxy-2-[[(1R)-2-hydroxy-1-[4-(4-methylthiazol-5-yl)phenyl]ethyl]carbamoyl]pyrrolidine-1-carbonyl]-2,2-dimethyl-propyl]amino]-3-oxo-propoxy]propyl]pyrrolidin-2-yl]methoxy]quinazolin-4-yl]-3,8-diazabicyclo[3.2.1]octane-8-carboxylate (45 mg, 0.03 mmol, 1 eq) in dichloromethane (1 mL) was added trifluoroacetic acid (1.54 g, 13.51 mmol, 1 mL, 406.69 eq). The mixture was stirred at 25° C. for 10 min. LCMS showed the reaction was completed. The reaction was concentrated in vacuum to give a residue which was purified by prep-HPLC (column: Phenomenex Synergi C18 150*25 mm*10 um; mobile phase: [water(FA)-ACN]; B %: 12%-34%, 11 min) to give the desired product (28 mg, 0.02 mmol, 64% yield, 95% purity, formate[3]) as a yellow solid. LCMS: (ESI, m/z): 1155.1[M]⁺. ¹H NMR: (400 MHz, DMSO-d₆) δ: 8.99 (s, 1H), 8.38 (d, J=8.0 Hz, 1H), 8.29 (s, 3H), 8.11 (s, 2H), 7.84 (s, 2H), 7.45-7.38 (m, 4H), 7.26 (dd, J=8.4, 5.6 Hz, 1H), 7.18-7.12 (m, 1H), 4.90-4.83 (m, 1H), 4.52 (br d, J=9.2 Hz, 1H), 4.46 (br t, J=8.4 Hz, 1H), 4.37-4.24 (m, 5H), 4.07-4.03 (m, 1H), 3.60-3.52 (m, 13H), 3.04-3.01 (m, 1H), 2.91-2.77 (m, 3H), 2.46-2.45 (m, 3H), 2.31-2.13 (m, 3H), 2.02-1.98 (m, 1H), 1.91 (br d, J=5.6 Hz, 1H), 1.84-1.80 (m, 1H), 1.71-1.57 (m, 10H), 0.90 (br s, 9H).

Synthesis of Intermediate 6

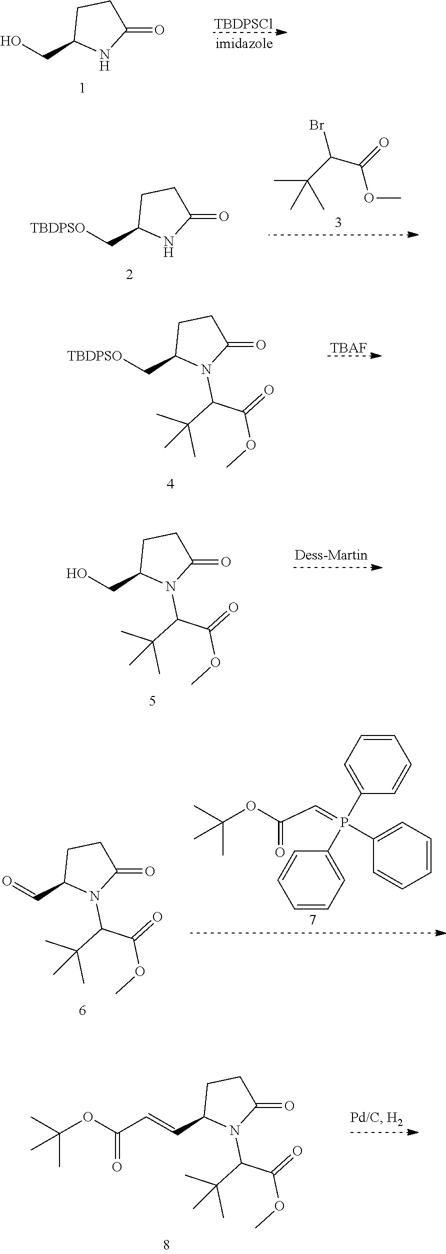

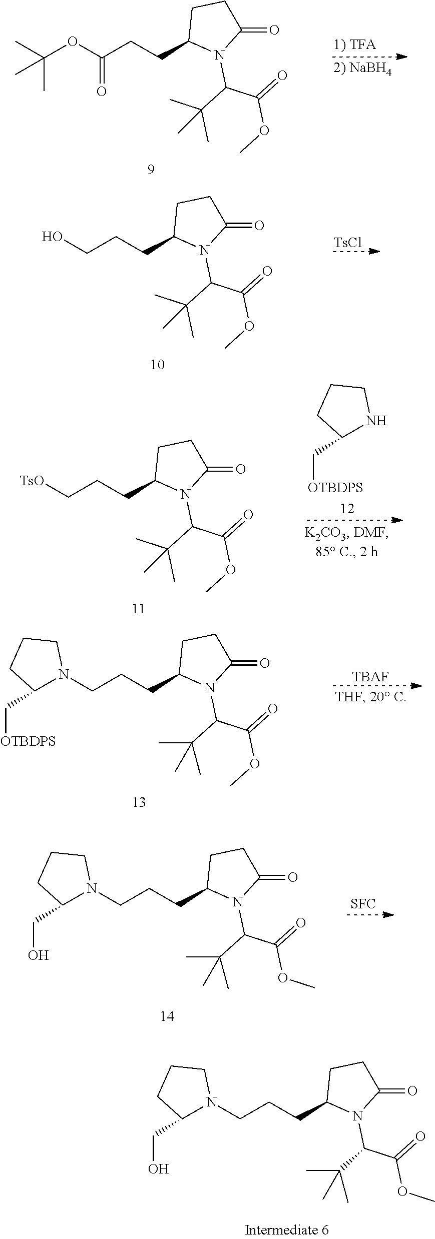

The alcohol in compound 1 is protected by treatment with TBDPSCl and a base such as imidazole to afford compound 2. Compound 2 is reacted with compound 3 in the presence of a base to give compound 4. Compound 5 is obtained by removing the protecting group in compound 4. Compound 5 is converted to compound 6 under Dess-Martin oxidation conditions. Compound 6 is converted to compound 8 following Wittig reaction conditions. The olefin in compound 8 is hydrogenated to afford compound 9. Compound 9 is converted to compound 10 by treatment with TFA and then NaBH₄. Compound 10 is treated with TsCl to generate compound 11. Reaction of compound 12 with compound 11 under basic conditions yields compound 13. The protecting group in compound 13 is removed by treatment with TBAF to give compound 14. The diasteromers of compound 14 are separated by SFC to afford Intermediate 6.

Synthesis of Example 10

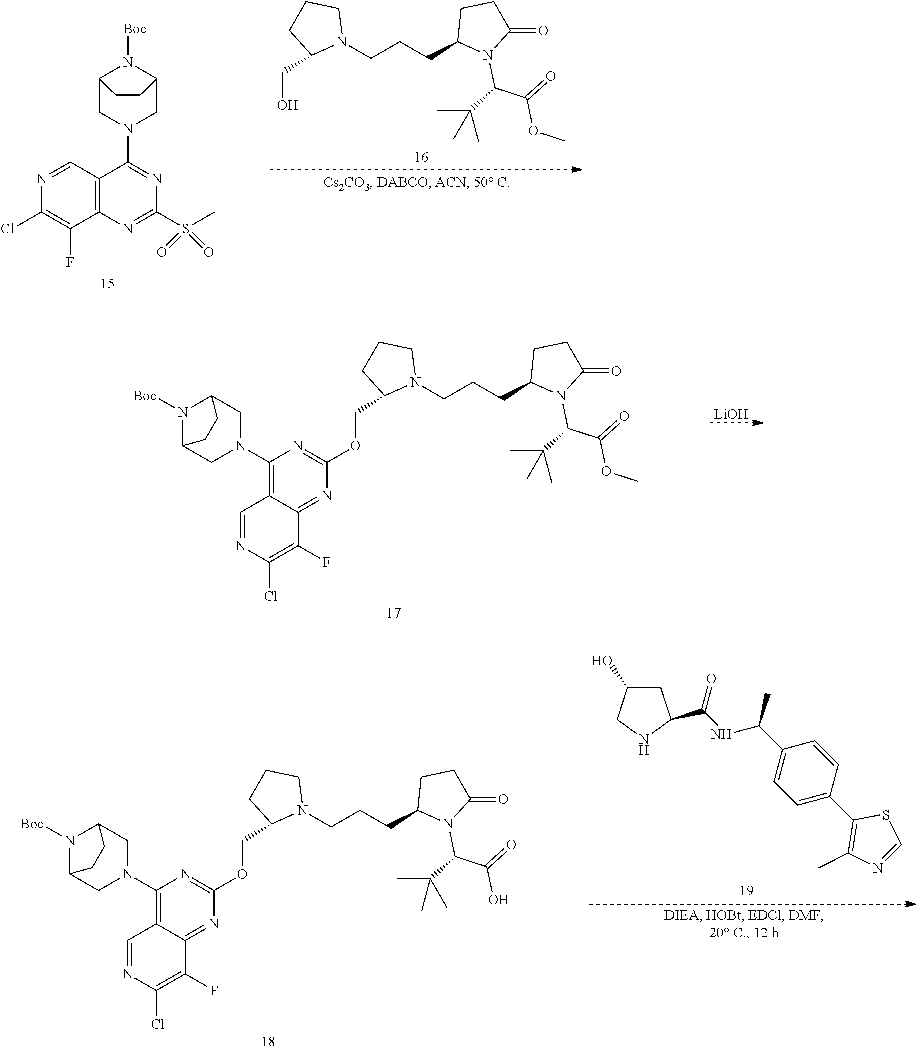 [SMILES: CC(C)(C)OC(=O)N1C2CCC1CN(c1nc(S(C)(=O)=O)nc3c(F)c(Cl)ncc13)C2 ; COC(=O)[C@@H](N1C(=O)CC[C@@H]1CCCN1CCC[C@H]1CO)C(C)(C)C ; COC(=O)[C@@H](N1C(=O)CC[C@@H]1CCCN1CCC[C@H]1COc1nc(N2CC3CCC(C2)N3C(=O)OC(C)(C)C)c2cnc(Cl)c(F)c2n1)C(C)(C)C ; "LiOH" ; CC(C)(C)OC(=O)N1C2CCC1CN(c1nc(OC[C@@H]3CCCN3CCC[C@H]3CCC(=O)N3[C@H](C(=O)O)C(C)(C)C)nc3c(F)c(Cl)ncc13)C2 ; Cc1ncsc1-c1ccc([C@H](C)NC(=O)[C@@H]2C[C@@H](O)CN2)cc1]

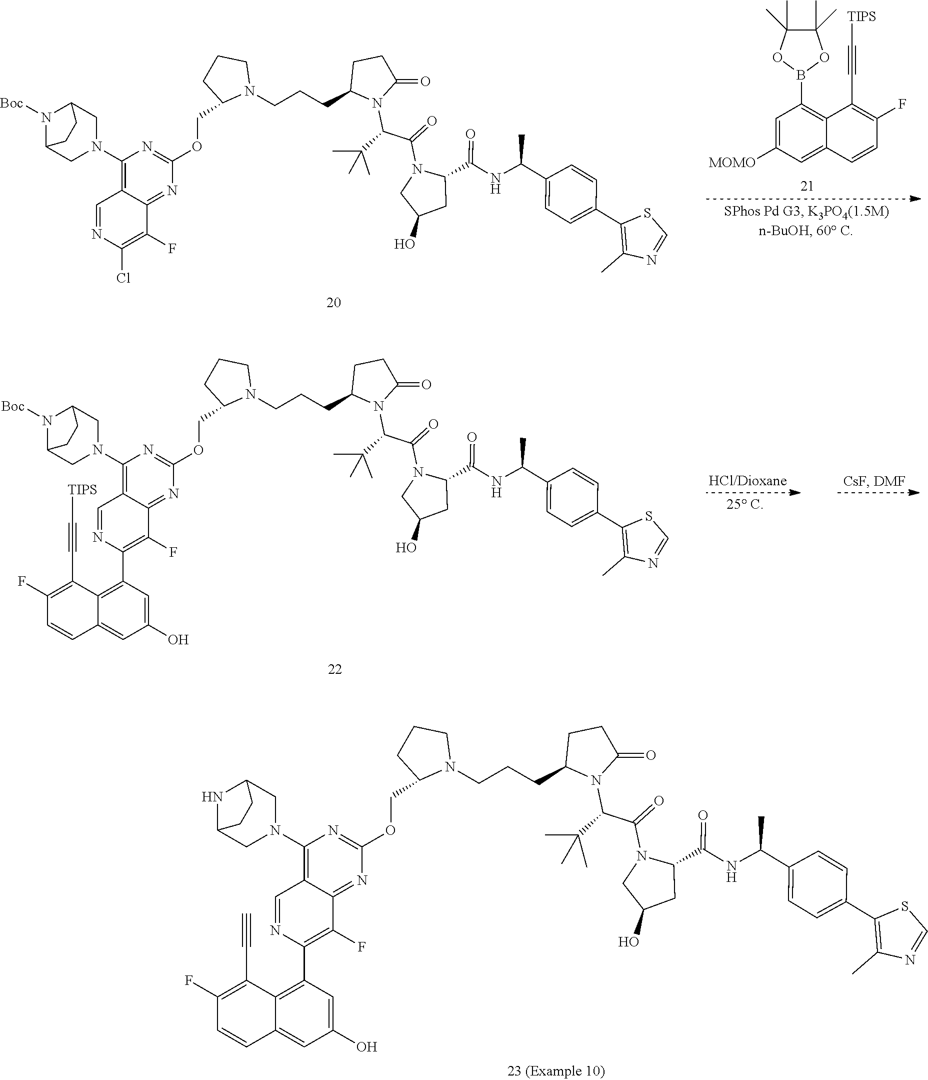

Compound 15 is reacted with compound 16 under basic conditions to afford compound 17. Compound 17 is treated with LiOH to generate compound 18. Compound 18 and compound 19 are coupled under amide coupling conditions to afford compound 20. Compound 20 and compound 21 are subjected to Suzuki coupling conditions to generate compound 22. Compound 22 is treated with HCl in dioxane to remove BOC group, and then treated with CsF to afford compound 23 (Example 10).

Example 10: (2S,4R)-1-((2S)-2-(2-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propyl)-5-oxopyrrolidin-1-yl)-3,3-dimethylbutanoyl)-4-hydroxy-N—((S)-1-(4-(4-methylthiazol-5-yl)phenyl)ethyl)pyrrolidine-2-carboxamide

Intermediate 1

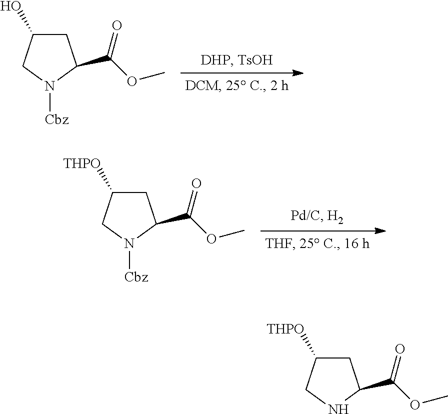

Intermediate 2

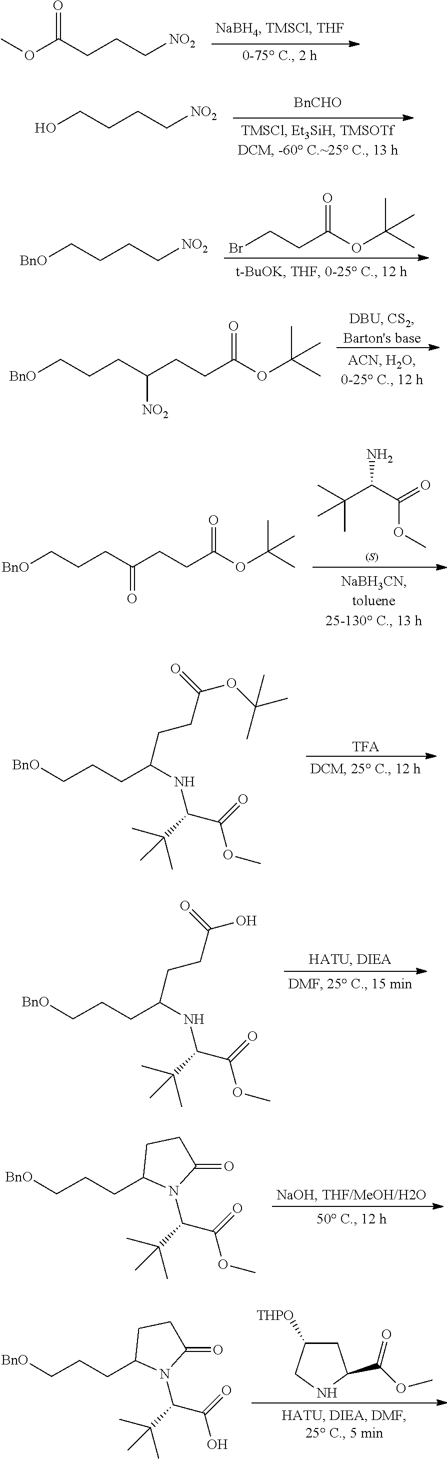

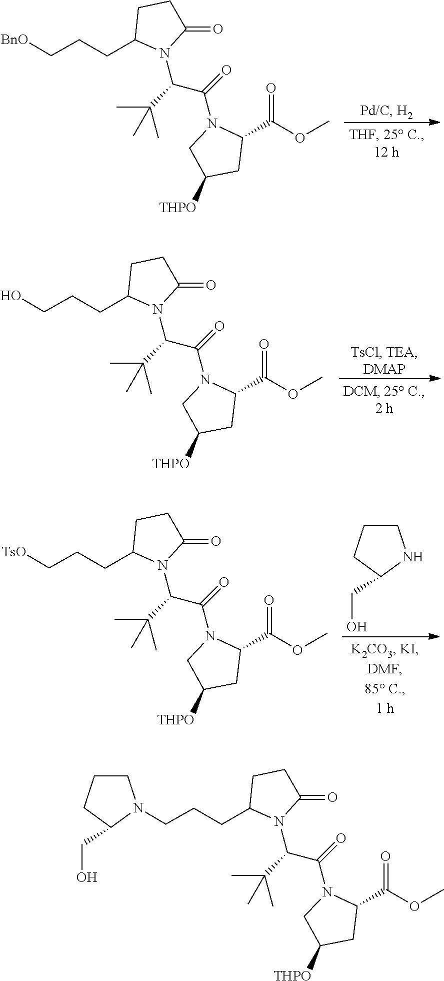

Example 10

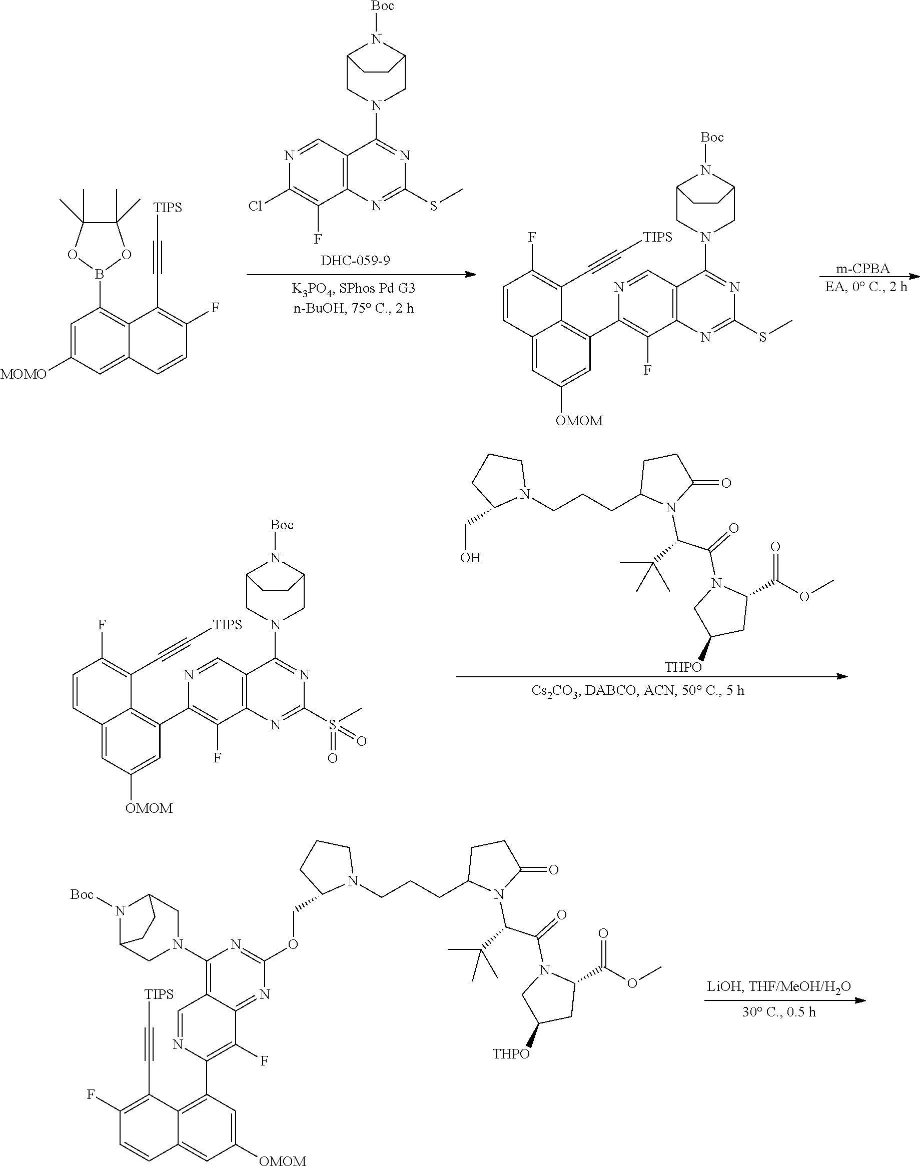

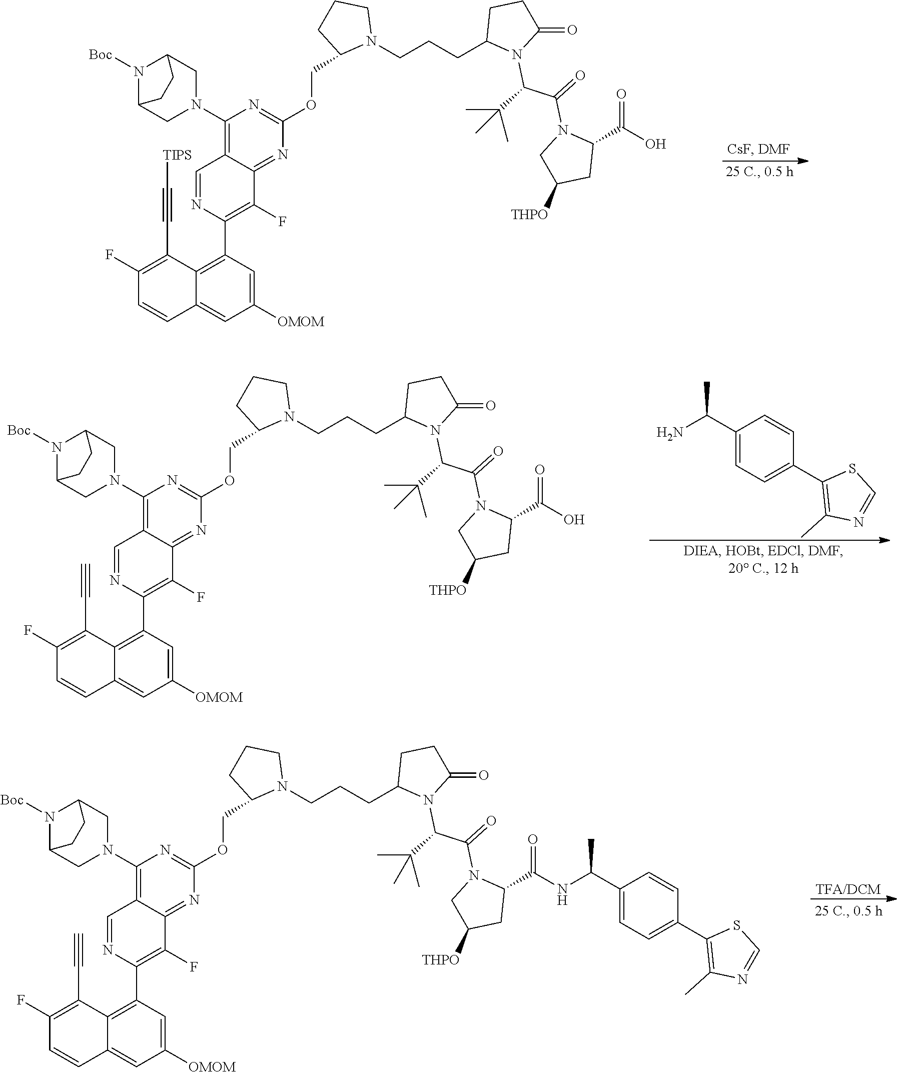

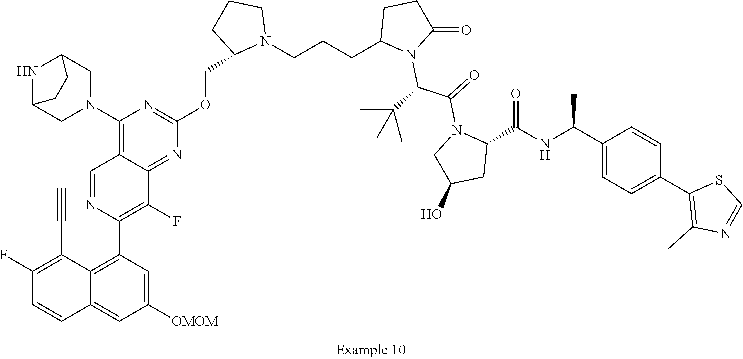

Step 1: 1-benzyl 2-methyl (2S,4R)-4-((tetrahydro-2H-pyran-2-yl)oxy)pyrrolidine-1,2-dicarboxylate

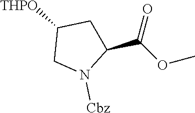

To a solution of 1-benzyl 2-methyl (2S,4R)-4-hydroxypyrrolidine-1,2-dicarboxylate (5 g, 17.90 mmol, 1 eq) in dichloromethane (80 mL) was added p-toluenesulfonic acid monohydrate (92 mg, 0.54 mmol, 0.03 eq) and 3,4-dihydro-2H-pyran (4.52 g, 53.71 mmol, 4.9 mL, 3 eq). The mixture was stirred at 25° C. for 2 h. LCMS showed the reaction was completed. The reaction mixture was added water (200 mL) and extracted with dichloromethane (100 mL×3). The combined organic phase was washed with brine (100 mL×2), dried over anhydrous sodium sulfate, filtered and concentrated in vacuum to get a residue. The residue was purified by prep-HPLC (column: Phenomenex luna C18 (250*70 mm, 10 um); mobile phase: [water(FA)-ACN]; B %: 36%-65%, 20 min) to afford the product (4.9 g, 13.48 mmol, 75% yield) as a yellow oil. LCMS (ESI, m/z): 364.1 [M+1]⁺, ¹H NMR (400 MHz, CDCl₃-d) δ 7.35-7.15 (m, 5H), 5.20-4.93 (m, 2H), 4.64-4.53 (m, 1H), 4.49-4.23 (m, 2H), 3.81-3.55 (m, 4H), 3.52-3.35 (m, 3H), 2.43-2.21 (m, 1H), 2.15-2.00 (m, 1H), 1.75-1.58 (m, 2H), 1.52-1.38 (m, 4H).

Step 2: methyl (2S,4R)-4-((tetrahydro-2H-pyran-2-yl)oxy)pyrrolidine-2-carboxylate

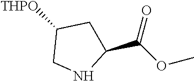

To a solution of 1-benzyl 2-methyl (2S,4R)-4-tetrahydropyran-2-yloxypyrrolidine-1,2-dicarboxylate (3 g, 8.26 mmol, 1 eq) in tetrahydrofuran (50 mL) was added palladium on activated carbon catalyst (500 mg, 5% purity) under nitrogen. The suspension was degassed under vacuum and purged with hydrogen several times. The mixture was stirred under hydrogen (50 psi) at 25° C. for 16 h. TLC showed the reaction was completed. The reaction mixture was filtered, and the filtrate was concentrated under reduced pressure to afford the crude product (1.8 g, 7.85 mmol, 95% yield) as a yellow oil, which was used in next step directly.

Step 3: 4-nitrobutan-1-ol

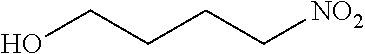

To a solution of methyl 4-nitrobutanoate (30 g, 203.9 mmol, 1.00 eq) in tetrahydrofuran (320 mL) was added sodium borohydride (23.12 g, 611.7 mmol, 3.00 eq) in nitrogen atmosphere at 0° C. Then trimethylchlorosilane (66.46 g, 611.7 mmol, 77.6 mL, 3.00 eq) was added slowly. The mixture was stirred at 75° C. for 2 h. TLC showed the reaction was completed. The reaction mixture was quenched by the addition of methanol (100 mL) at 10° C. slowly for 1 h. Then water (500 mL) was added before the mixture was extracted with ethyl acetate (250 mL×2). The combined organic layers were washed with brine (500 mL), dried over sodium sulfate, filtered and concentrated under reduced pressure to afford the crude product 4-nitrobutan-1-ol (18.6 g, 156.15 mmol, 76% yield) as a yellow oil. ¹H NMR (400 MHz, CHLOROFORM-d) δ: 4.45 (t, J=7.2 Hz, 2H), 3.70 (t, J=6.0 Hz, 2H), 2.21-2.05 (m, 2H), 1.72-1.60 (m, 2H).

Step 4: (4-nitrobutoxy)methyl)benzene

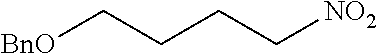 [SMILES: O=[N+]([O-])CCCCOCc1ccccc1]

To a solution of 4-nitrobutan-1-ol (18.6 g, 156.15 mmol, 1.00 eq) in tetrahydrofuran (400 mL) was added triethylamine (18.17 g, 179.57 mmol, 25 mL, 1.15 eq) and trimethylchlorosilane (18.66 g, 171.76 mmol, 21.8 mL, 1.10 eq) at 0° C. The mixture was stirred at 25° C. for 1 h. The reaction mixture was filtered, and the filtrate was concentrated under reduced pressure to give a residue. Then to a stirred solution of above residue and benzaldehyde (17.90 g, 168.64 mmol, 17 mL, 1.08 eq) in dichloromethane (200 mL) was added triethylsilane (20.88 g, 179.57 mmol, 28.7 mL, 1.15 eq), trimethylsilyl trifluoromethanesulfonate (17.35 g, 78.07 mmol, 14.1 mL, 0.50 eq) dropwise at −60° C., the mixture was stirred at 25° C. in nitrogen atmosphere for 12 h. TLC showed the reaction was completed. The reaction mixture was quenched by the addition of saturated aqueous sodium bicarbonate (200 mL) at 0° C., and then diluted with ethyl acetate (200 mL). The mixture was extracted with ethyl acetate (200 mL×3), and the combined organic layers were washed with brine (500 mL), dried over sodium sulfate, filtered and concentrated under reduced pressure to give a residue. The residue was purified by silica gel column chromatography (0-20% ethyl acetate in petroleum ether) to afford the product 4-nitrobutoxymethylbenzene (29.37 g, 140.36 mmol, 90% yield) as a colorless oil. ¹H NMR (400 MHz, CHLOROFORM-d) δ: 7.47-7.25 (m, 5H), 4.53 (s, 2H), 4.44 (t, J=7.2 Hz, 2H), 3.54 (t, J=6.0 Hz, 2H), 2.22-2.12 (m, 2H), 1.78-1.69 (m, 2H).

Step 5: tert-butyl 7-(benzyloxy)-4-nitroheptanoate

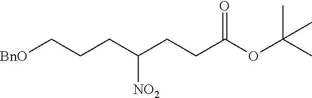 [SMILES: CC(C)(C)OC(=O)CCC(CCCOCc1ccccc1)[N+](=O)[O-]]

To a solution of 4-nitrobutoxymethylbenzene (15 g, 71.69 mmol, 1.00 eq) and tert-butyl 3-bromopropanoate (29.98 g, 143.38 mmol, 24 mL, 2.00 eq) in tetrahydrofuran (150 mL) was added potassium tert-butoxide (16.09 g, 143.38 mmol, 2.00 eq) at 0° C. The mixture was stirred at 25° C. for 12 h. TLC showed the reaction was completed. The reaction mixture was quenched by adding saturated aqueous ammonium chloride (1000 mL) at 25° C., and then extracted with ethyl acetate (200 mL×2). The combined organic layers were washed with brine (400 mL), dried over sodium sulfate, filtered and concentrated under reduced pressure to give a residue. The residue was purified by silica gel column chromatography (0-10% ethyl acetate in petroleum ether) to afford the product tert-butyl 7-benzyloxy-4-nitro-heptanoate (24 g, 49.79 mmol, 69% yield, 70% purity) as a colorless oil. ¹H NMR (400 MHz, CHLOROFORM-d) δ: 7.41-7.27 (m, 5H), 4.59 (tt, J =4.4, 9.2 Hz, 1H), 4.51 (s, 2H), 3.56-3.45 (m, 2H), 2.37-2.24 (m, 2H), 2.19-2.06 (m, 2H), 2.06-1.99 (m, 1H), 1.98-1.87 (m, 1H), 1.74-1.59 (m, 2H), 1.46 (d, J=2.0 Hz, 9H).

Step 6: tert-butyl 7-(benzyloxy)-4-oxoheptanoate

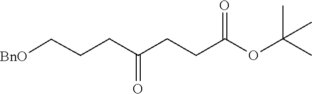 [SMILES: CC(C)(C)OC(=O)CCC(=O)CCCOCc1ccccc1]

To a solution of tert-butyl 7-benzyloxy-4-nitro-heptanoate (9 g, 26.67 mmol, 1 eq) in acetonitrile (400 mL) was added carbon disulfide (12.19 g, 160.04 mmol, 9.7 mL, 6 eq) and 1,8-diazabicyclo[5.4.0]undec-7-ene (6.09 g, 40.01 mmol, 6 mL, 1.5 eq), and the mixture was stirred at 0° C. for 0.5 h, then 2-tert-butyl-1,1,3,3-tetramethyl-guanidine (6.85 g, 40.01 mmol, 8 mL, 1.5 eq) was added before the mixture was stirred for another 5 min at 0° C. The resultant mixture was stirred at 25° C. for 3 h, then water (128 g, 7.14 mol, 128 mL, 267.56 eq) was added before the mixture was stirred at 25° C. for 12 h. TLC (Petroleum ether/Ethyl acetate=3/1) showed the reaction was completed. The reaction mixture was extracted with ethyl acetate (200 mL×3). The combined organic phase was washed with brine (100 mL×3), dried over anhydrous sodium sulfate, filtered and concentrated in vacuum. The residue was purified by prep-HPLC (column: Phenomenex luna c18 250 mm*100 mm*10 um; mobile phase: [water(TFA)-ACN]; B %: 45%-75%, 20 min) to afford the desired product tert-butyl 7-benzyloxy-4-oxo-heptanoate (2.1 g, 6.85 mmol, 25% yield) as a yellow oil. ¹H NMR (400 MHz, CDCl₃-d) δ: 7.39-7.28 (m, 5H), 4.48 (s, 2H), 3.49 (t, J=6.4 Hz, 2H), 2.70-2.64 (m, 2H), 2.58 (t, J=7.2 Hz, 2H), 2.52-2.46 (m, 2H), 1.92 (quin, J=6.8 Hz, 2H), 1.44 (s, 9H).

Step 7: tert-butyl 7-(benzyloxy)-4-(((S)-1-methoxy-3,3-dimethyl-1-oxobutan-2-yl)amino)heptanoate

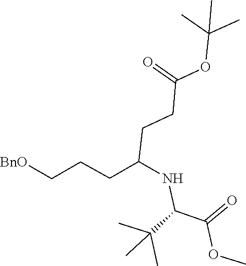 [SMILES: COC(=O)[C@@H](NC(CCCOCc1ccccc1)CCC(=O)OC(C)(C)C)C(C)(C)C]

To a solution of methyl (2S)-2-amino-3,3-dimethyl-butanoate (4.84 g, 26.63 mmol, 1.2 eq, hydrochloride) in toluene (100 mL) was added diisopropylethylamine (8.61 g, 66.58 mmol, 11.6 mL, 3 eq) and tert-butyl 7-benzyloxy-4-oxo-heptanoate (6.8 g, 22.19 mmol, 1 eq). The mixture was stirred at 130° C. for 12 h. Then sodium cyanoborohydride (9.41 g, 44.39 mmol, 2 eq) was added before the mixture was stirred at 25° C. for 1 h. LCMS showed the desired mass was detected. The reaction mixture was added water (200 mL) and extracted with ethyl acetate (100 mL×3). The combined organic phase was washed with brine (100 mL×2), dried over anhydrous sodium sulfate, filtered and concentrated in vacuum. The residue was purified by prep-HPLC (column: Phenomenex luna c18 250 mm*100 mm*10 um; mobile phase: [water(TFA)-ACN]; B %: 35%-60%, 20 min) to afford the product (2.6 g, 5.97 mmol, 26% yield) as a yellow oil. LCMS (ESI, m/z): 436.1 [M+1]⁺, ¹H NMR (400 MHz, CHLOROFORM-d) δ: 7.38-7.27 (m, 5H), 4.50 (d, J=2.4 Hz, 2H), 3.67 (d, J=11.2 Hz, 3H), 3.51-3.39 (m, 2H), 2.93 (s, 1H), 2.44-2.15 (m, 3H), 1.73-1.52 (m, 5H), 1.44 (s, 9H), 1.40 (br d, J=7.2 Hz, 2H), 0.94 (s, 9H).

Step 8: 7-(benzyloxy)-4-(((S)-1-methoxy-3,3-dimethyl-1-oxobutan-2-yl)amino)heptanoic acid

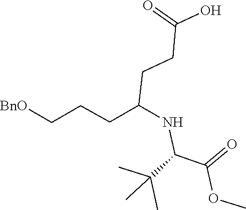 [SMILES: COC(=O)[C@@H](NC(CCCOCc1ccccc1)CCC(=O)O)C(C)(C)C]

To a solution of tert-butyl 7-benzyloxy-4-[[(1S)-1-methoxy carbonyl-2,2-dimethyl-propyl] amino] heptanoate (2.6 g, 5.97 mmol, 1 eq) in dichloromethane (10 mL) was added trifluoroacetic acid (7.70 g, 67.53 mmol, 5 mL, 11.31 eq). The mixture was stirred at 25° C. for 12 h. LCMS showed the reaction was completed. The reaction mixture was concentrated in reduced pressure to give the product (2.2 g, 5.80 mmol, 97% yield) as a yellow oil, which was used into next step without purification LCMS (ESI, m/z): 380.0 [M+1]⁺.

Step 9: methyl (2S)-2-(2-(3-(benzyloxy)propyl)-5-oxopyrrolidin-1-yl)-3,3-dimethylbutanoate

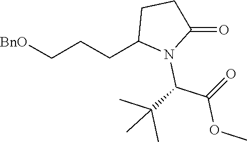 [SMILES: COC(=O)[C@@H](N1C(=O)CCC1CCCOCc1ccccc1)C(C)(C)C]

To a solution of 7-benzyloxy-4-[[(1S)-1-methoxycarbonyl-2,2-dimethyl-propyl]amino]heptanoic acid (2.2 g, 5.80 mmol, 1 eq) in N,N-dimethylformamide (50 mL) was added N,N-diisopropylethylamine (2.25 g, 17.39 mmol, 3 mL, 3 eq) and o-(7-azabenzotriazol-1-yl)-n,n,n′,n′-tetramethyluronium hexafluorophosphate (3.31 g, 8.70 mmol, 1.5 eq). The mixture was stirred at 25° C. for 15 min. LCMS showed the reaction was completed. The reaction mixture was added water (250 mL) and extracted with ethyl acetate (50 mL×3). The combined organic phase was washed with brine (50 mL×2), dried over anhydrous sodium sulfate, filtered and concentrated in vacuum. The residue was purified by silica gel chromatography (Petroleum ether/Ethyl acetate=1/0 to 2/1) to afford the product (1.5 g, 4.15 mmol, 71% yield) as a colorless oil. LCMS (ESI, m/z): 362.3 [M+1]⁺, ¹H NMR (400 MHz, CHLOROFORM-d) δ 7.40-7.28 (m, 5H), 4.51 (d, J=12.8 Hz, 2H), 3.64 (d, J=14.4 Hz, 4H), 3.55-3.44 (m, 2H), 2.51-2.10 (m, 3H), 1.92-1.28 (m, 6H), 1.10 (d, J=8.8 Hz, 9H).

Step 10: (2S)-2-(2-(3-(benzyloxy)propyl)-5-oxopyrrolidin-1-yl)-3,3-dimethylbutanoic acid.

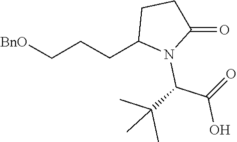 [SMILES: CC(C)(C)[C@@H](C(=O)O)N1C(=O)CCC1CCCOCc1ccccc1]

To a solution of methyl (2S)-2-[2-(3-benzyloxypropyl)-5-oxo-pyrrolidin-1-yl]-3,3-dimethyl-butanoate (1.4 g, 3.87 mmol, 1 eq) in tetrahydrofuran (4 mL), methanol (4 mL) was added sodium hydroxide (4 M, 9.7 mL, 10 eq). The mixture was stirred at 50° C. for 12 h. TLC showed the reaction was completed. pH of the reaction mixture was adjusted to 6-7 by adding aqueous hydrogen chloride (1 M), then the mixture was extracted with ethyl acetate (100 mL×2). The combined organic layers were washed with brine (50 mL×3), dried over anhydrous sodium sulfate, filtered and concentrated in vacuum to get the crude product (1.3 g, 3.74 mmol, 96% yield) as a colorless oil, which was used into next step without purification. LCMS (ESI, m/z): 348.3 [M+1]⁺.

Step 11: methyl (2S,4R)-1-((2S)-2-(2-(3-(benzyloxy)propyl)-5-oxopyrrolidin-1-yl)-3,3-dimethylbutanoyl)-4-((tetrahydro-2H-pyran-2-yl)oxy)pyrrolidine-2-carboxylate

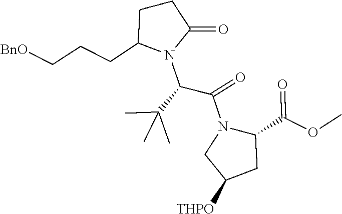

To a solution of (2S)-2-[2-(3-benzyloxypropyl)-5-oxo-pyrrolidin-1-yl]-3,3-dimethyl-butanoic acid (4 g, 11.51 mmol, 1 eq) and methyl (2S,4R)-4-tetrahydropyran-2-yloxypyrrolidine-2-carboxylate (3.17 g, 13.82 mmol, 1.2 eq) in N,N-dimethylformamide (50 mL) was added triethylamine (3.49 g, 34.54 mmol, 4.81 mL, 3 eq) and o-(7-azabenzotriazol-1-yl)-n,n,n′,n′-tetramethyluronium hexafluorophosphate (6.57 g, 17.27 mmol, 1.5 eq). The mixture was stirred at 25° C. for 5 min. LCMS showed the reaction was completed. Water (200 mL) was added before the mixture was extracted with dichloromethane (100 mL×3). The combined organic phase was washed with brine (50 mL×3), dried over anhydrous sodium sulfate, filtered and concentrated in vacuum to get a residue. The residue was purified by prep-HPLC (column: Phenomenex luna C18 (250*70 mm, 10 um); mobile phase: [water(TFA)-ACN]; B %: 40%-80%, 25 min) to afford the product (2 g, 3.58 mmol, 31% yield) as a yellow oil. LCMS (ESI, m/z): 559.2 [M+1]⁺.

Step 12: methyl (2S,4R)-1-((2S)-2-(2-(3-hydroxypropyl)-5-oxopyrrolidin-1-yl)-3,3-dimethylbutanoyl)-4-((tetrahydro-2H-pyran-2-yl)oxy)pyrrolidine-2-carboxylate

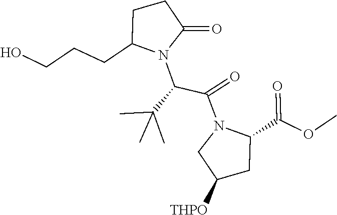

To a solution of methyl (2S,4R)-1-[(2S)-2-[2-(3-benzyloxypropyl)-5-oxo-pyrrolidin-1-yl]-3,3-dimethyl-butanoyl]-4-tetrahydropyran-2-yloxy-pyrrolidine-2-carboxylate (2 g, 3.58 mmol, 1 eq) in tetrahydrofuran (20 mL) was added palladium on activated carbon catalyst (300 mg, 3.58 mmol, 5% purity) under nitrogen. The suspension was degassed under vacuum and purged with hydrogen several times. The mixture was stirred under hydrogen (50 psi) at 25° C. for 12 h. TLC showed the reaction was completed. The reaction mixture was filtered and concentrated under reduced pressure to afford the product (1.7 g, crude) as a yellow oil, which was used into next step without further purification. LCMS (ESI, m/z): 468.5 [M+1]⁺.

Step 13: methyl (2S,4R)-1-((2S)-3,3-dimethyl-2-(2-oxo-5-(3-(tosyloxy)propyl)pyrrolidin-1-yl)butanoyl)-4-((tetrahydro-2H-pyran-2-yl)oxy)pyrrolidine-2-carboxylate.

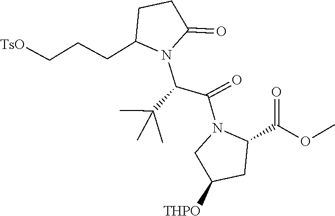

To a solution of methyl (2S,4R)-1-[(2S)-2-[2-(3-hydroxypropyl)-5-oxo-pyrrolidin-1-yl]-3,3-dimethyl-butanoyl]-4-tetrahydropyran-2-yloxy-pyrrolidine-2-carboxylate (1.7 g, 3.63 mmol, 1 eq) in dichloromethane (20 mL) was added p-toluenesulfonyl chloride (1.04 g, 5.44 mmol, 1.5 eq) and triethylamine (1.10 g, 10.88 mmol, 1.5 mL, 3 eq), dimethylaminopyridine (44.32 mg, 0.36 mmol, 0.1 eq). The mixture was stirred at 25° C. for 2 h. LCMS showed the reaction was completed. Water (100 mL) was added before the reaction mixture was extracted with ethyl acetate (200 mL×3). The combined organic phase was washed with brine (50 mL×2), dried over anhydrous sodium sulfate, filtered and concentrated in vacuum to get a residue. The residue was purified by silica gel chromatography (dichloromethane/methanol=100/1 to 10/1) to afford the product (1.2 g, 1.93 mmol, 53% yield) as a yellow oil. LCMS (ESI, m/z): 623.1 [M+1]⁺.

Step 14: methyl (2S,4R)-1-((2S)-2-(2-(3-((S)-2-(hydroxymethyl)pyrrolidin-1-yl)propyl)-5-oxopyrrolidin-1-yl)-3,3-dimethylbutanoyl)-4-((tetrahydro-2H-pyran-2-yl)oxy)pyrrolidine-2-carboxylate

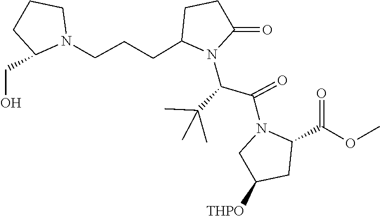

A mixture of [(2S)-pyrrolidin-2-yl]methanol (390 mg, 3.85 mmol, 2 eq), methyl (2S,4R)-1-[(2S)-3,3-dimethyl-2-[2-oxo-5-[3-(p-tolylsulfonyloxy)propyl]pyrrolidin-1-yl]butanoyl]-4-tetrahydropyran-2-yloxy-pyrrolidine-2-carboxylate (1.2 g, 1.93 mmol, 1 eq), potassium carbonate (799 mg, 5.78 mmol, 3 eq) and potassium iodide (32 mg, 0.19 mmol, 0.1 eq) in acetonitrile (30 mL) was degassed and purged with nitrogen for 3 times, and then the mixture was stirred at 85° C. for 1 h under nitrogen. LCMS showed the reaction was completed. Water (100 mL) was added before the reaction mixture was extracted with ethyl acetate (60 mL×3). The combined organic phase was washed with brine (50 mL×2), dried over anhydrous sodium sulfate, filtered and concentrated in vacuum to get a residue. The residue was purified by prep-HPLC (column: Phenomenex luna C18 250*50 mm*10 um; mobile phase: [water(HCl)-ACN]; B %: 20%-50%, 20 min) to afford the desired product (700 mg, 1.27 mmol, 66% yield) as a white solid. LCMS (ESI, m/z): 552.4 [M+1]⁺.

Step 15: tert-butyl (1R,5S)-3-(8-fluoro-7-(7-fluoro-3-(methoxymethoxy)-8-((triisopropylsilyl)ethynyl)naphthalen-1-yl)-2-(methylthio)pyrido[4,3-d]pyrimidin-4-yl)-3,8-diazabicyclo[3.2.1]octane-8-carboxylate

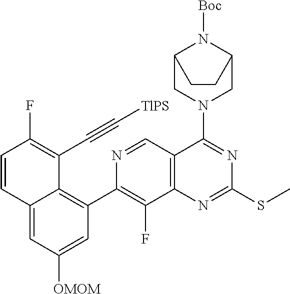

To a solution of 2-[2-fluoro-6-(methoxymethoxy)-8-(4,4,5,5-tetramethyl-1,3,2-dioxaborolan-2-yl)-1-naphthyl]ethynyl-triisopropyl-silane (6.4 g, 12.49 mmol, 1.2 eq), tert-butyl 3-(7-chloro-8-fluoro-2-methylsulfanyl-pyrido[4,3-d]pyrimidin-4-yl)-3,8-diazabicyclo [3.2.1]octane-8-carboxylate (4.58 g, 10.41 mmol, 1 eq) in butan-1-ol (200 mL) was added potassium phosphate (1.5 M, 34.7 mL, 5 eq) and dicyclohexyl-[2-(2,6-dimethoxyphenyl)phenyl] phosphane; methanesulfonate; (2-phenylanilino)palladium (811 mg, 1.04 mmol, 0.1 eq). The mixture was stirred at 75° C. for 2 h. LCMS showed the reaction was completed. The reaction mixture was extracted with ethyl acetate (50 mL×3). The combined organic phase was washed with brine (50 mL×2), dried over anhydrous anhydrous sodium sulfate, filtered and concentrated in vacuum. The residue was purified by silica gel chromatography (Petroleum ether/Ethyl acetate=1/0 to 3/1) to afford the product (6.1 g, 7.72 mmol, 74% yield) as a yellow solid. LCMS (ESI, m/z): 790.2 [M]⁺, ¹H NMR (400 MHz, CHLOROFORM-d) δ=8.96 (s, 1H), 7.71 (dd, J=5.6, 9.2 Hz, 1H), 7.44 (d, J=2.8 Hz, 1H), 7.27-7.17 (m, 2H), 5.29-5.14 (m, 2H), 4.75 (br d, J=3.6 Hz, 1H), 4.46-4.22 (m, 2H), 3.87-3.65 (m, 1H), 3.43 (s, 3H), 3.34 (br d, J=7.6 Hz, 1H), 2.55 (s, 3H), 1.95-1.87 (m, 3H), 1.66-1.49 (m, 2H), 1.45 (s, 9H), 1.17 (s, 3H), 0.80 (d, J=7.6 Hz, 18H).

Step 16: tert-butyl (1R,5S)-3-(8-fluoro-7-(7-fluoro-3-(methoxymethoxy)-8-((triisopropylsilyl)ethynyl)naphthalen-1-yl)-2-(methylsulfonyl)pyrido[4,3-d]pyrimidin-4-yl)-3,8-diazabicyclo[3.2.1]octane-8-carboxylate.

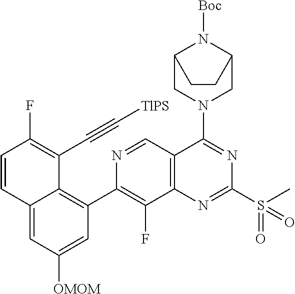

To a solution of tert-butyl 3-[8-fluoro-7-[7-fluoro-3-(methoxymethoxy)-8-(2-triisopropylsilylethynyl)-1-naphthyl]-2-methylsulfanyl-pyrido [4,3-d]pyrimidin-4-yl]-3,8-diazabicyclo[3.2.1]octane-8-carboxylate (6 g, 7.59 mmol, 1 eq) in ethyl acetate (60 mL) was added 3-chloro-benzenecarboperoxoic acid (4.63 g, 22.78 mmol, 85% purity, 3 eq). The mixture was stirred at 0° C. for 2 h. LCMS showed the reaction was completed. The mixture was quenched with saturated aqueous sodium sulfite (100 mL), extracted with ethyl acetate (100 mL×3). The organic layer was washed with brine (30 mL×2), dried over sodium sulfate, filtered and then concentrated under vacuum to get a residue. The residue was purified by silica gel chromatography (Petroleum ether/Ethyl acetate=1/0 to 2/1) to afford the product (5 g, 6.08 mmol, 80% yield) as a yellow solid. LCMS (ESI, m/z): 822.4 [M]⁺.

Step 17: tert-butyl 3-(8-fluoro-7-(7-fluoro-3-(methoxymethoxy)-8-((triisopropylsilyl)ethynyl)naphthalen-1-yl)-2-(((2S)-1-(3-(1-((2S)-1-((2S,4R)-2-(methoxycarbonyl)-4-((tetrahydro-2H-pyran-2-yl)oxy)pyrrolidin-1-yl)-3,3-dimethyl-1-oxobutan-2-yl)-5-oxopyrrolidin-2-yl)propyl)pyrrolidin-2-yl)methoxy)pyrido[4,3-d]pyrimidin-4-yl)-3,8-diazabicyclo[3.2.1]octane-8-carboxylate

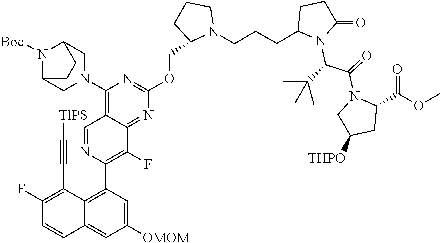

To a solution of tert-butyl 3-[8-fluoro-7-[7-fluoro-3-(methoxymethoxy)-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate (372 mg, 0.45 mmol, 1 eq) and methyl (2S,4R)-1-[(2S)-2-[2-[3-[(2S)-2-(hydroxymethyl)pyrrolidin-1-yl]propyl]-5-oxo-pyrrolidin-1-yl]-3,3-dimethyl-butanoyl]-4-tetrahydropyran-2-yloxy-pyrrolidine-2-carboxylate (300 mg, 0.54 mmol, 1.2 eq) in acetonitrile (3 mL) was added 1,4-diazabicyclo[2.2.2]octane (5 mg, 0.04 mmol, 0.1 eq) and cesium carbonate (443 mg, 1.36 mmol, 3 eq). The mixture was stirred at 50° C. for 5 h. LCMS showed the reaction was completed. Water (30 mL) was added before the reaction mixture was extracted with ethyl acetate (60 mL×3). The combined organic phase was washed with brine (50 mL×2), dried over anhydrous sodium sulfate, filtered and concentrated in vacuum to get a residue. The residue was purified by prep-TLC (9% methanol in dichloromethane) to afford the product (100 mg, 0.08 mmol, 17% yield) as a yellow oil. LCMS (ESI, m/z): 1292.4 [M]⁺.

Step 18: (2S,4R)-1-((2S)-2-(2-(3-((2S)-2-(((4-(8-(tert-butoxycarbonyl)-3,8-diazabicyclo[3.2.1]octan-3-yl)-8-fluoro-7-(7-fluoro-3-(methoxymethoxy)-8-((triisopropylsilyl)ethynyl)naphthalen-1-yl)pyrido[4,3-d]pyrimidin-2-yl)oxy)methyl)pyrrolidin-1-yl)propyl)-5-oxopyrrolidin-1-yl)-3,3-dimethylbutanoyl)-4-((tetrahydro-2H-pyran-2-yl)oxy)pyrrolidine-2-carboxylic acid

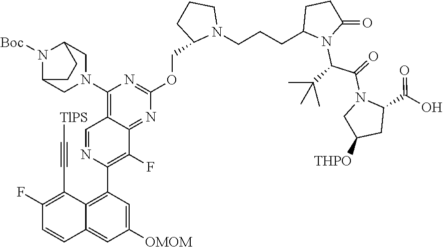

To a solution of tert-butyl 3-[8-fluoro-7-[7-fluoro-3-(methoxymethoxy)-8-(2-triisopropylsilylethynyl)-1-naphthyl]-2-[[(2S)-1-[3-[1-[(1S)-1-[(2S,4R)-2-methoxycarbonyl-4-tetrahydropyran-2-yloxy-pyrrolidine-1-carbonyl]-2,2-dimethyl-propyl]-5-oxo-pyrrolidin-2-yl]propyl]pyrrolidin-2-yl]methoxy]pyrido[4,3-d]pyrimidin-4-yl]-3,8-diazabicyclo[3.2.1]octane-8-carboxylate (20 mg, 0.015 mmol, 1 eq) in methanol (0.5 mL) and tetrahydrofuran (0.5 mL) was added lithium hydroxide (2 M, 0.15 mL, 20 eq). The mixture was stirred at 30° C. for 0.5 h. LCMS showed the reaction was completed. The mixture was adjusted pH to 5-6 with hydrogen chloride (0.5 M). Then the mixture was extracted with ethyl acetate (10 mL×3). The combined organic layers were washed with brine (20 mL×2), dried over anhydrous sodium sulfate, filtered and concentrated in vacuum to give the product (20 mg, crude) as a yellow solid, which was used into the next step without further purification. LCMS (ESI, m/z): 1279.5 [M]⁺.

Step 19: (2S,4R)-1-((2S)-2-(2-(3-((2S)-2-(((4-(8-(tert-butoxycarbonyl)-3,8-diazabicyclo[3.2.1]octan-3-yl)-7-(8-ethynyl-7-fluoro-3-(methoxymethoxy)naphthalen-1-yl)-8-fluoropyrido[4,3-d]pyrimidin-2-yl)oxy)methyl)pyrrolidin-1-yl)propyl)-5-oxopyrrolidin-1-yl)-3,3-dimethylbutanoyl)-4-((tetrahydro-2H-pyran-2-yl)oxy)pyrrolidine-2-carboxylic acid

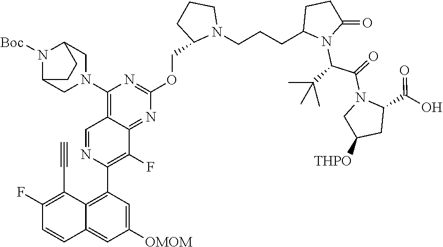

To a solution of (2S,4R)-1-[(2S)-2-[2-[3-[(2S)-2-[[4-(8-tert-butoxycarbonyl-3,8-diazabicyclo[3.2.1]octan-3-yl)-8-fluoro-7-[7-fluoro-3-(methoxymethoxy)-8-(2-triisopropylsilylethynyl)-1-naphthyl]pyrido[4,3-d]pyrimidin-2-yl]oxymethyl]pyrrolidin-1-yl]propyl]-5-oxo-pyrrolidin-1-yl]-3,3-dimethyl-butanoyl]-4-tetrahydropyran-2-yloxy-pyrrolidine-2-carboxylic acid (150 mg, 0.12 mmol, 1 eq) in N,N-dimethylformamide (2 mL) was added cesium fluoride (267 mg, 1.76 mmol, 15 eq). The mixture was stirred at 25° C. for 0.5 h. LCMS showed the reaction was completed. The reaction mixture was diluted with water (40 mL) and extracted with ethyl acetate (50 mL×2). The combined organic phase was washed with brine (100 mL), dried over anhydrous sodium sulfate, filtered and concentrated in vacuum to get the crude product (120 mg, 0.11 mmol, 91% yield) as a yellow solid, which was used into next step without further purification. LCMS (ESI, m/z): 1123.5 [M]⁺.

Step 20: tert-butyl 3-(2-(((2S)-1-(3-(1-((2S)-3,3-dimethyl-1-((2S,4R)-2-(((S)-1-(4-(4-methylthiazol-5-yl)phenyl)ethyl)carbamoyl)-4-((tetrahydro-2H-pyran-2-yl)oxy)pyrrolidin-1-yl)-1-oxobutan-2-yl)-5-oxopyrrolidin-2-yl)propyl)pyrrolidin-2-yl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate

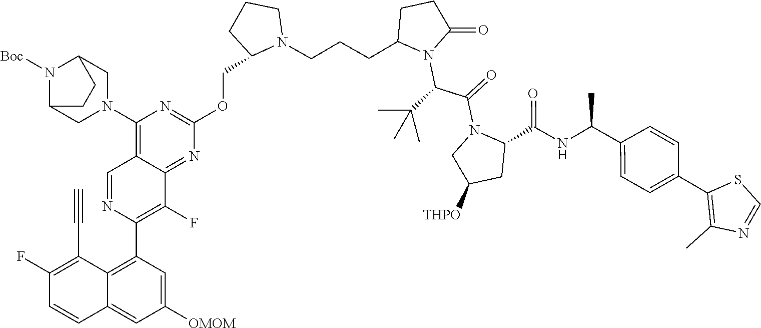

To a solution of (1S)-1-[4-(4-methylthiazol-5-yl)phenyl]ethanamine (41 mg, 0.16 mmol, 1.5 eq, hydrochloride) and (2S,4R)-1-[(2S)-2-[2-[3-[(2S)-2-[[4-(8-tert-butoxycarbonyl-3,8-diazabicyclo[3.2.1]octan-3-yl)-7-[8-ethynyl-7-fluoro-3-(methoxymethoxy)-1-naphthyl]-8-fluoro-pyrido[4,3-d]pyrimidin-2-yl]oxymethyl]pyrrolidin-1-yl]propyl]-5-oxo-pyrrolidin-1-yl]-3,3-dimethyl-butanoyl]-4-tetrahydropyran-2-yloxy-pyrrolidine-2-carboxylic acid (120 mg, 0.11 mmol, 1 eq) in N,N-dimethylformamide (2 mL) was added diisopropylethylamine (69 mg, 0.53 mmol, 0.1 mL, 5 eq) and N-(3-dimethylaminopropyl)-N-ethylcarbodiimide hydrochloride (61 mg, 0.32 mmol, 3 eq), 1-hydroxybenzotriazole (43 mg, 0.32 mmol, 3 eq). The mixture was stirred at 20° C. for 12 h. LCMS showed the reaction was completed. The reaction mixture was diluted with water (50 mL) and extracted with dichloromethane (40 mL×3). The combined organic layers were washed with brine (50 mL), dried over anhydrous sodium sulfate, filtered and concentrated under reduced pressure to give a residue. The residue was purified by prep-TLC (9% methanol in dichloromethane) to afford the product (60 mg, 0.04 mmol, 43% yield) as a white solid. LCMS (ESI, m/z): 1324.0 [M]⁺.

Step 21: (2S,4R)-1-((2S)-2-(2-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propyl)-5-oxopyrrolidin-1-yl)-3,3-dimethylbutanoyl)-4-hydroxy-N—((S)-1-(4-(4-methylthiazol-5-yl)phenyl)ethyl)pyrrolidine-2-carboxamide (Example 10)

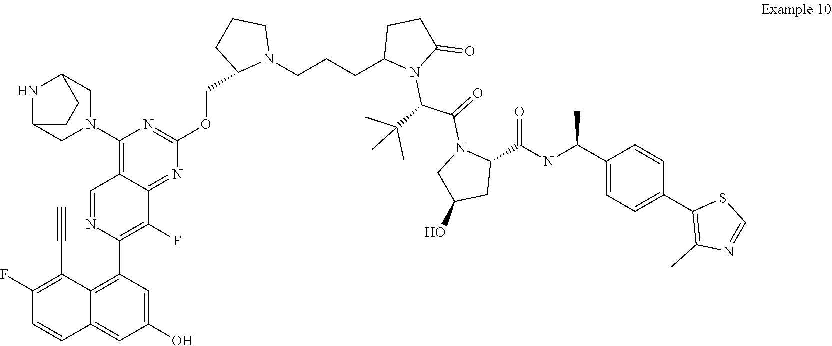

To a solution of tert-butyl 3-[2-[[(2S)-1-[3-[1-[(1S)-2,2-dimethyl-1-[(2S,4R)-2-[[(1S)-1-[4-(4-methylthiazol-5-yl)phenyl]ethyl]carbamoyl]-4-tetrahydropyran-2-yloxy-pyrrolidine-1-carbonyl]propyl]-5-oxo-pyrrolidin-2-yl]propyl]pyrrolidin-2-yl]methoxy]-7-[8-ethynyl-7-fluoro-3-(methoxymethoxy)-1-naphthyl]-8-fluoro-pyrido[4,3-d]pyrimidin-4-yl]-3,8-diazabicyclo[3.2.1]octane-8-carboxylate (60 mg, 0.04 mmol, 1 eq) in dichloromethane (1 mL) was added trifluoroacetic acid (1.54 g, 13.51 mmol, 1 mL, 297.94 eq). The mixture was stirred at 25° C. for 0.5 h. LCMS showed the reaction was completed. The reaction mixture was concentrated under reduced pressure to give a residue. The residue was purified by prep-HPLC (column: Phenomenex Synergi Polar-RP 100*25 mm*4 um; mobile phase: [water(TFA)-ACN]; B %: 31%-51%, 7 min) to afford the desired product (10.6 mg, 0.01 mmol, 21% yield, 96% purity) as a yellow solid. LCMS (ESI, m/z): 1095.3 [M]⁺, ¹H NMR (400 MHz, DMSO) δ:10.29-10.00 (m, 1H), 9.04 (s, 1H), 9.00-8.97 (m, 1H), 8.51-8.25 (m, 1H), 7.97 (dd, J=6.0, 9.2 Hz, 1H), 7.51-7.29 (m, 6H), 7.18 (s, 1H), 5.14-5.03 (m, 1H), 4.98-4.85 (m, 1H), 4.57 (s, 1H), 4.52-4.20 (m, 6H), 4.17-4.03 (m, 1H), 3.95-3.90 (m, 1H), 3.84-3.72 (m, 1H), 3.69-3.51 (m, 6H), 3.09-3.03 (m, 2H), 2.90-2.78 (m, 2H), 2.45-2.41 (m, 3H), 2.30-2.25 (m, 1H), 2.14-2.03 (m, 3H), 1.96-1.91 (m, 1H), 1.68-1.62 (m, 4H), 1.55-1.46 (m, 4H), 1.43-1.41 (m, 1H), 1.37-1.32 (m, 3H), 1.17-1.11 (m, 3H), 1.00-0.95 (m, 9H), 0.75-0.68 (m, 2H).

Synthesis of Example 11

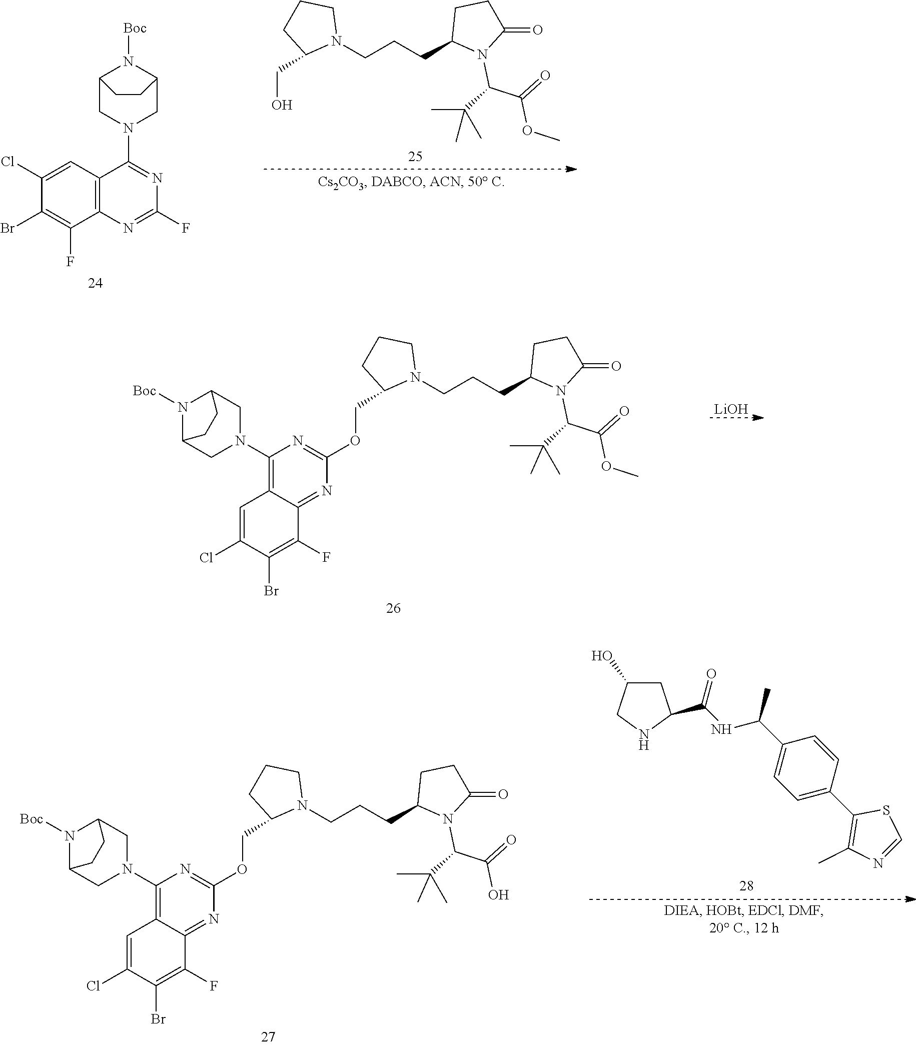 [SMILES: CC(C)(C)OC(=O)N1C2CCC1CN(c1nc(F)nc3c(F)c(Br)c(Cl)cc13)C2 ; COC(=O)[C@@H](N1C(=O)CC[C@@H]1CCCN1CCC[C@H]1CO)C(C)(C)C ; COC(=O)[C@@H](N1C(=O)CC[C@@H]1CCCN1CCC[C@H]1COc1nc(N2CC3CCC(C2)N3C(=O)OC(C)(C)C)c2cc(Cl)c(Br)c(F)c2n1)C(C)(C)C ; "LiOH" ; CC(C)(C)OC(=O)N1C2CCC1CN(c1nc(OC[C@@H]3CCCN3CCC[C@H]3CCC(=O)N3[C@H](C(=O)O)C(C)(C)C)nc3c(F)c(Br)c(Cl)cc13)C2 ; Cc1ncsc1-c1ccc([C@H](C)NC(=O)[C@@H]2C[C@@H](O)CN2)cc1]

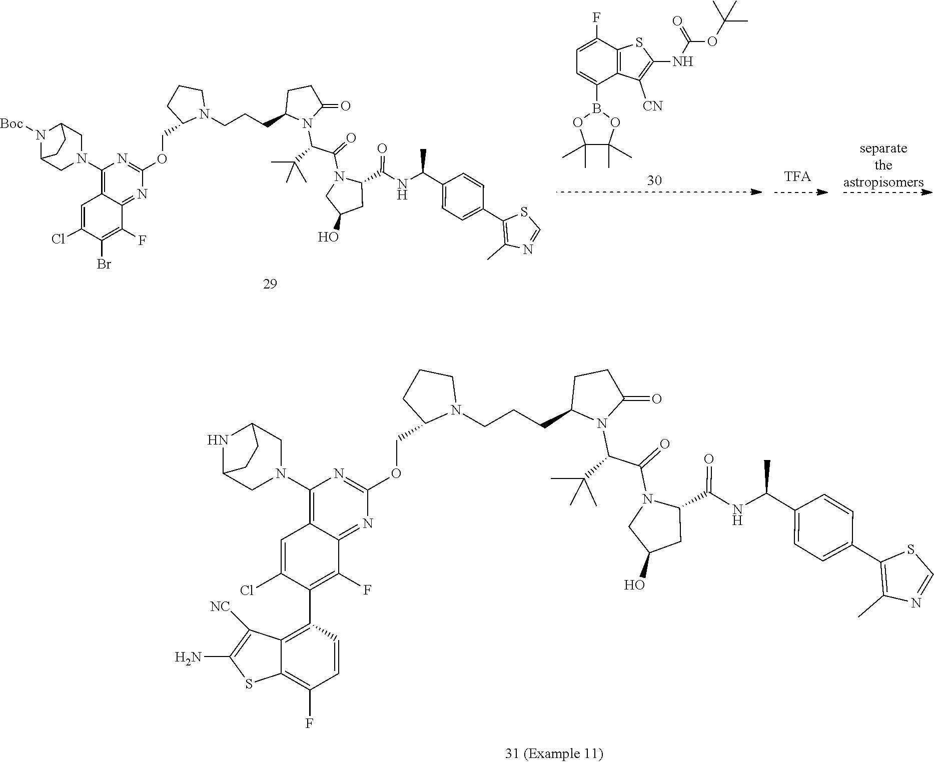

Compound 24 is reacted with compound 25 under basic conditions to afford compound 26. Compound 26 is treated with LiOH to generate compound 27. Compound 27 and compound 28 are coupled under amide coupling conditions to afford compound 29. Compound 29 and compound 30 are subjected to Suzuki coupling conditions, the resultant product is then treated with TFA to remove BOC group, and the atropisomers are separated by SFC to afford compound 31 (Example 11).

Synthesis of Intermediate 7: tert-butyl 3-[8-fluoro-7-[7-fluoro-3-(methoxymethoxy)-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate

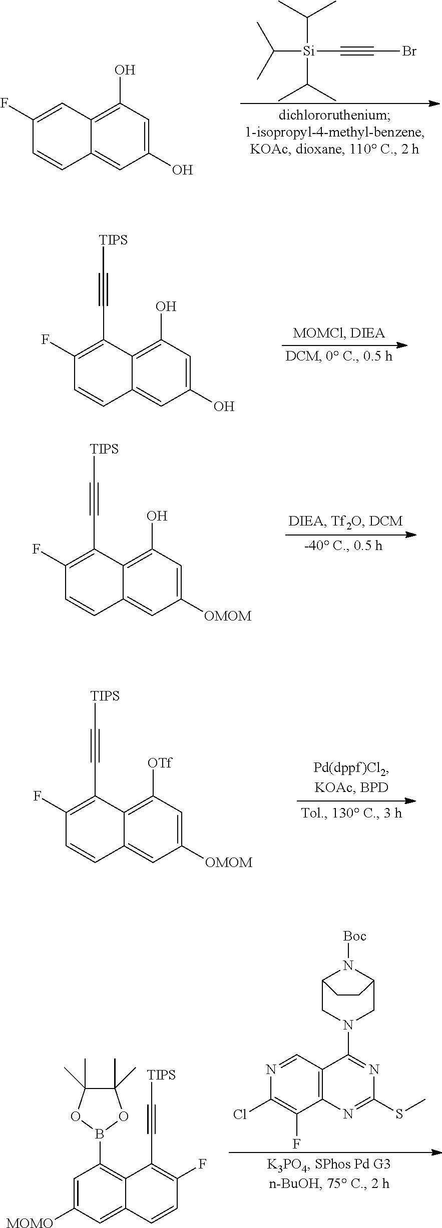

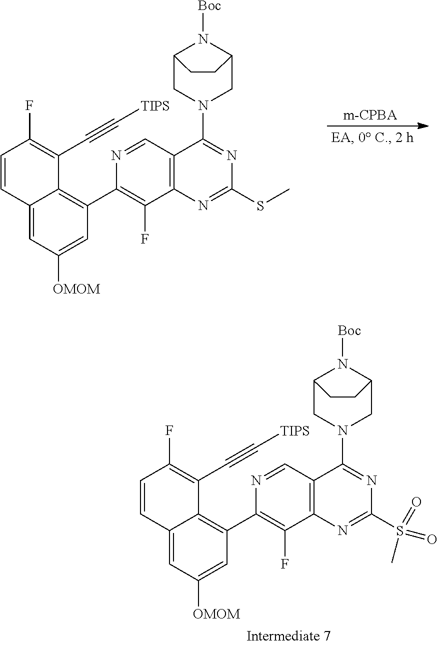

Step 1: 7-fluoro-8-(2-triisopropylsilylethynyl)naphthalene-1,3-diol

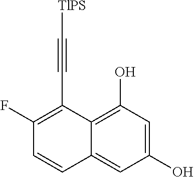

To a solution of 2-bromoethynyl(triisopropyl)silane (35.20 g, 134.71 mmol, 1.2 eq), 7-fluoronaphthalene-1,3-diol (20 g, 112.26 mmol, 1 eq) in dioxane (300 mL) was added potassium acetate (22.03 g, 224.52 mmol, 2 eq) and dichlororuthenium; 1-isopropyl-4-methyl-benzene (6.87 g, 11.23 mmol, 0.1 eq). The mixture was stirred at 110° C. for 2 h. The reaction mixture was filtered and extracted with ethyl acetate (200 mL×3). The combined organic layers were washed with brine (100 mL×2), dried with anhydrous sodium sulfate, filtered and concentrated in vacuum. The residue was purified by silica gel chromatography (0-33% ethyl acetate in petroleum ether) to afford the desired product (40 g, 111.57 mmol, 99% yield) as a yellow solid. LCMS (ESI, m/z): 359.3 [M]⁺.

Step 2: 7-fluoro-3-(methoxymethoxy)-8-(2-triisopropylsilylethynyl)naphthalen-1-ol

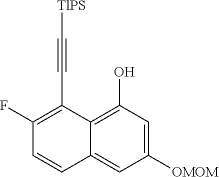

To a solution of 7-fluoro-8-(2-triisopropylsilylethynyl)naphthalene-1,3-diol (20 g, 55.78 mmol, 1 eq) in dichloromethane (200 mL) was added diisopropylethylamine (21.63 g, 167.35 mmol, 29 mL, 3 eq) and chloromethyl methyl ether (6.01 g, 74.67 mmol, 5.7 mL, 1.34 eq). The mixture was stirred at 0° C. for 0.5 h under nitrogen. The reaction mixture was quenched by pouring it into water (500 ml) slowly then the mixture was extracted with ethyl acetate (200 mL×3). The combined organic layers were washed with brine (100 mL×2), dried with anhydrous sodium sulfate, filtered and concentrated in vacuum. The residue was purified by silica gel chromatography (0-10% ethyl acetate in petroleum ether) to give the desired product (13 g, 32.29 mmol, 57% yield) as a yellow solid. ¹H NMR: (400 MHz, CHLOROFORM-d) δ: 9.13 (s, 1H), 7.66 (dd, J=5.6, 9.2 Hz, 1H), 7.21-7.16 (m, 1H), 6.97 (d, J=2.4 Hz, 1H), 6.81 (d, J=2.4 Hz, 1H), 5.25 (s, 2H), 3.51 (s, 3H), 1.21-1.18 (m, 21H).

Step 3: [7-fluoro-3-(methoxymethoxy)-8-(2-triisopropylsilylethynyl)-1-naphthyl] trifluoromethanesulfonate

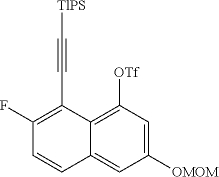

To a solution of 7-fluoro-3-(methoxymethoxy)-8-(2-triisopropylsilylethynyl)naphthalen-1-ol (13 g, 32.29 mmol, 1 eq) in dichloromethane (140 mL) was added diisopropylethylamine (12.52 g, 96.88 mmol, 16.8 mL, 3 eq) and trifluoromethanesulfonic anhydride (13.67 g, 48.44 mmol, 8 mL, 1.5 eq). The mixture was stirred at −40° C. for 0.5 h. The reaction mixture was poured into water (200 ml) then the mixture was extracted with ethyl acetate (100 mL×3). The combined organic phase was washed with brine (50 mL×2), dried with anhydrous sodium sulfate, filtered and concentrated in vacuum. The residue was purified by silica gel chromatography (0-10% ethyl acetate in petroleum ether) to afford the desired product (15.1 g, 28.24 mmol, 87% yield) as a yellow solid. ¹H NMR: (400 MHz, CDCl₃) δ: 7.71 (dd, J=5.6, 9.2 Hz, 1H), 7.43 (d, J=2.4 Hz, 1H), 7.36 (d, J=2.4 Hz, 1H), 7.34-7.29 (m, 1H), 5.28 (s, 2H), 3.52 (s, 3H), 1.21-1.15 (m, 21H).

Step 4: 2-[2-fluoro-6-(methoxymethoxy)-8-(4,4,5,5-tetramethyl-1,3,2-dioxaborolan-2-yl)-1-naphthyl]ethynyl-triisopropyl-silane

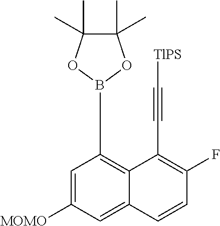

To a solution of [7-fluoro-3-(methoxymethoxy)-8-(2-triisopropylsilylethynyl)-1-naphthyl] trifluoromethanesulfonate (11 g, 20.57 mmol, 1 eq) in toluene (100 mL) was added 4,4,5,5-tetramethyl-2-(4,4,5,5-tetramethyl-1,3,2-dioxaborolan-2-yl)-1,3,2-dioxaborolane (11.49 g, 45.26 mmol, 2.2 eq) and [1,1-Bis(diphenylphosphino)ferrocene]dichloropalladium(II) (1.51 g, 2.06 mmol, 0.1 eq), potassium acetate (7.07 g, 72.01 mmol, 3.5 eq). The mixture was stirred at 130° C. for 3 h under nitrogen. The reaction mixture was poured into water (500 mL), then extracted with ethyl acetate (100 mL×3). The combined organic phase was washed with brine (100 mL×2), dried with anhydrous sodium sulfate, filtered and concentrated in vacuum. The residue was purified by silica gel chromatography (0-10% ethyl acetate in petroleum ether) and triturated by acetonitrile (40 ml) to give the desired product (3.4 g, 6.63 mmol, 32% yield) as a white solid. ¹H NMR: (400 MHz, CDCl₃) δ: 7.67 (dd, J=5.6, 9.2 Hz, 1H), 7.51 (d, J=2.8 Hz, 1H), 7.38 (d, J=2.8 Hz, 1H), 7.23 (t, J=8.8 Hz, 1H), 5.28 (s, 2H), 3.51 (s, 3H), 1.44 (s, 12H), 1.22-1.13 (m, 21H).

Step 5: tert-butyl 3-[8-fluoro-7-[7-fluoro-3-(methoxymethoxy)-8-(2-triisopropylsilylethynyl)-1-naphthyl]-2-methylsulfanyl-pyrido[4,3-d]pyrimidin-4-yl]-3,8-diazabicyclo [3.2.1]octane-8-carboxylate

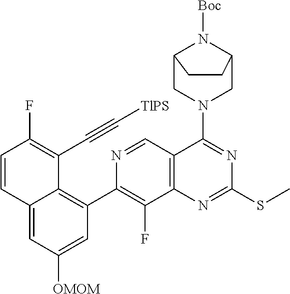

To a solution of 2-[2-fluoro-6-(methoxymethoxy)-8-(4,4,5,5-tetramethyl-1,3,2-dioxaborolan-2-yl)-1-naphthyl]ethynyl-triisopropyl-silane (6.4 g, 12.49 mmol, 1.2 eq), tert-butyl 3-(7-chloro-8-fluoro-2-methylsulfanyl-pyrido[4,3-d]pyrimidin-4-yl)-3,8-diazabicyclo [3.2.1]octane-8-carboxylate (4.58 g, 10.41 mmol, 1 eq) in butan-1-ol (200 mL) was added potassium phosphate (1.5 M, 34.7 mL, 5 eq) and dicyclohexyl-[2-(2,6-dimethoxyphenyl)phenyl] phosphane; methanesulfonate; (2-phenylanilino)palladium (811 mg, 1.04 mmol, 0.1 eq). The mixture was stirred at 75° C. for 2 h. The reaction mixture was poured into water (500 ml) then extracted with ethyl acetate (100 mL×3). The combined organic phase was washed with brine (100 mL×2), dried with anhydrous sodium sulfate, filtered and concentrated in vacuum. The residue was purified by silica gel chromatography (0-33% ethyl acetate in petroleum ether) to give the desired product (6.1 g, 7.72 mmol, 74% yield) as a yellow solid. LCMS (ESI, m/z): 790.2[M+1]⁺. ¹H NMR: (400 MHz, CDCl₃) δ: 8.96 (s, 1H), 7.71 (dd, J=5.6, 9.2 Hz, 1H), 7.44 (d, J=2.8 Hz, 1H), 7.27-7.17 (m, 2H), 5.29-5.14 (m, 2H), 4.75 (br d, J=3.6 Hz, 1H), 4.46-4.22 (m, 2H), 3.87-3.65 (m, 1H), 3.43 (s, 3H), 3.34 (br d, J=7.6 Hz, 1H), 2.55 (s, 3H), 1.95-1.87 (m, 3H), 1.66-1.49 (m, 2H), 1.45 (s, 9H), 1.17 (s, 3H), 0.80 (d, J=7.6 Hz, 18H).

Step 6: tert-butyl 3-[8-fluoro-7-[7-fluoro-3-(methoxymethoxy)-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate

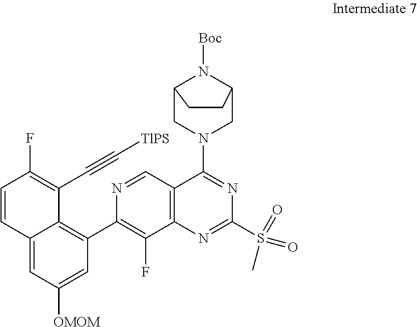

To a solution of tert-butyl 3-[8-fluoro-7-[7-fluoro-3-(methoxymethoxy)-8-(2-triisopropylsilylethynyl)-1-naphthyl]-2-methylsulfanyl-pyrido [4,3-d]pyrimidin-4-yl]-3,8-diazabicyclo[3.2.1]octane-8-carboxylate (6 g, 7.59 mmol, 1 eq) in ethyl acetate (60 mL) was added 3-chloro-benzenecarboperoxoic acid (4.63 g, 22.78 mmol, 85% purity, 3 eq). The mixture was stirred at 0° C. for 2 h. The mixture was quenched with saturated aqueous sodium sulfite (100 mL), extracted with ethyl acetate (100 mL×3). The combined organic layers were washed with brine (30 mL×2), dried over sodium sulfate, filtered and then concentrated under vacuum to get a residue. The residue was purified by s silica gel chromatography (0-50% ethyl acetate in petroleum ether) to afford the desired product (5 g, 6.08 mmol, 80% yield) as a yellow solid. LCMS (ESI, m/z): 822.4[M+1]⁺.

Synthesis of Intermediate 8: (2S,4R)-1-[(2S)-2-amino-3,3-dimethyl-butanoyl]-N-[(1S)-1-[4-(2,6-difluorophenyl)phenyl]ethyl]-4-hydroxy-pyrrolidine-2-carboxamide

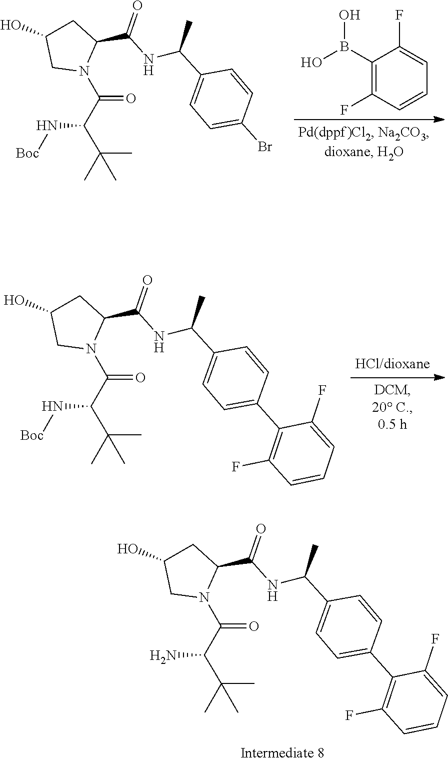 [SMILES: C[C@H](NC(=O)[C@@H]1C[C@@H](O)CN1C(=O)[C@@H](NC(=O)OC(C)(C)C)C(C)(C)C)c1ccc(Br)cc1 ; OB(O)c1c(F)cccc1F ; C[C@H](NC(=O)[C@@H]1C[C@@H](O)CN1C(=O)[C@@H](NC(=O)OC(C)(C)C)C(C)(C)C)c1ccc(-c2c(F)cccc2F)cc1 ; C[C@H](NC(=O)[C@@H]1C[C@@H](O)CN1C(=O)[C@@H](N)C(C)(C)C)c1ccc(-c2c(F)cccc2F)cc1]

Step 1: tert-butyl N-[1-[(2S,4R)-2-[[(1S)-1-[4-(2,6-difluorophenyl)phenyl]ethyl]carbamoyl]-4-hydroxy-pyrrolidine-1-carbonyl]-2,2-dimethyl-propyl]carbamate

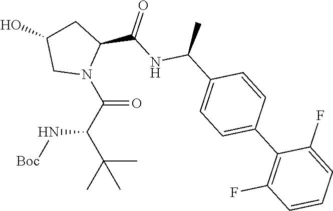 [SMILES: C[C@H](NC(=O)[C@@H]1C[C@@H](O)CN1C(=O)[C@@H](NC(=O)OC(C)(C)C)C(C)(C)C)c1ccc(-c2c(F)cccc2F)cc1]

To a solution of tert-butyl N-[1-[(2S,4R)-2-[[(1S)-1-(4-bromophenyl)ethyl]carbamoyl]-4-hydroxy-pyrrolidine-1-carbonyl]-2,2-dimethyl-propyl]carbamate (1.00 g, 1.90 mmol, 1.00 eq) in dioxane (10 mL) and water (2 mL) was added (2,6-difluorophenyl)boronic acid (450 mg, 2.85 mmol, 1.50 eq), [1,1′-bis(diphenylphosphino)ferrocene]dichloropalladium(II) (139 mg, 0.20 mmol, 0.10 eq) and sodium carbonate (604 mg, 5.70 mmol, 3 eq). The mixture was stirred at 90° C. for 12 h under nitrogen atmosphere. The reaction mixture was partitioned between water (20 mL) and ethyl acetate (20 mL). The organic phase was separated, washed with brine (20 mL×3), dried over sodium sulfate, filtered and the filtrate was concentrated under reduced pressure to give a residue. The residue was purified by silica gel chromatography (Petroleum ether/Ethyl acetate=1/0 to 1/1) to get the desired product (600 mg, 1.07 mmol, 56% yield) as a yellow oil. LCMS (ESI, m/z): 560.4 [M+H]⁺.

Step 2: (2S,4R)-1-[(2S)-2-amino-3,3-dimethyl-butanoyl]-N-[(1S)-1-[4-(2,6-difluorophenyl)phenyl]ethyl]-4-hydroxy-pyrrolidine-2-carboxamide

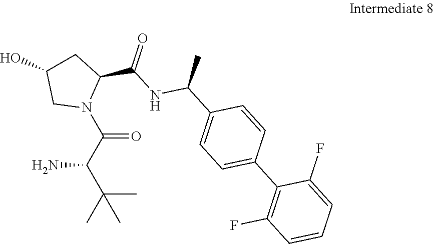 [SMILES: C[C@H](NC(=O)[C@@H]1C[C@@H](O)CN1C(=O)[C@@H](N)C(C)(C)C)c1ccc(-c2c(F)cccc2F)cc1]

To a solution of tert-butyl N-[1-[(2S,4R)-2-[[(1S)-1-[4-(2,6-difluorophenyl)phenyl]ethyl]carbamoyl]-4-hydroxy-pyrrolidine-1-carbonyl]-2,2-dimethyl-propyl]carbamate (400 mg, 0.7 mmol, 1 eq) in dichloromethane (5 mL) was added hydrochloride/dioxane (4 M, 1.0 mL, 6 eq). The mixture was stirred at 20° C. for 0.5 h. The reaction mixture was filtered and concentrated under reduced pressure to give a residue. The residue was purified by prep-HPLC (column: Phenomenex luna C18 150*40 mm*15 um; mobile phase: [water(FA)-ACN]; B %: 15%-45%, 10 min) to get the desired product (80 mg, 0.17 mmol, 24% yield) as a yellow oil. LCMS (ESI, m/z): 460.4 [M+1]⁺.

Example 12: (2S,4R)-1-[(2S)-2-[3-[3-[2-[[7-(2-amino-3-cyano-7-fluoro-benzothiophen-4-yl)-6-chloro-4-(3,8-diazabicyclo[3.2.1]octan-3-yl)-8-fluoro-quinazolin-2-yl]oxymethyl]pyrrolidin-1-yl]propoxy]propanoylamino]-3,3-dimethyl-butanoyl]-N-[(1S)-1-[4-(2,6-difluorophenyl)phenyl]ethyl]-4-hydroxy-pyrrolidine-2-carboxamide

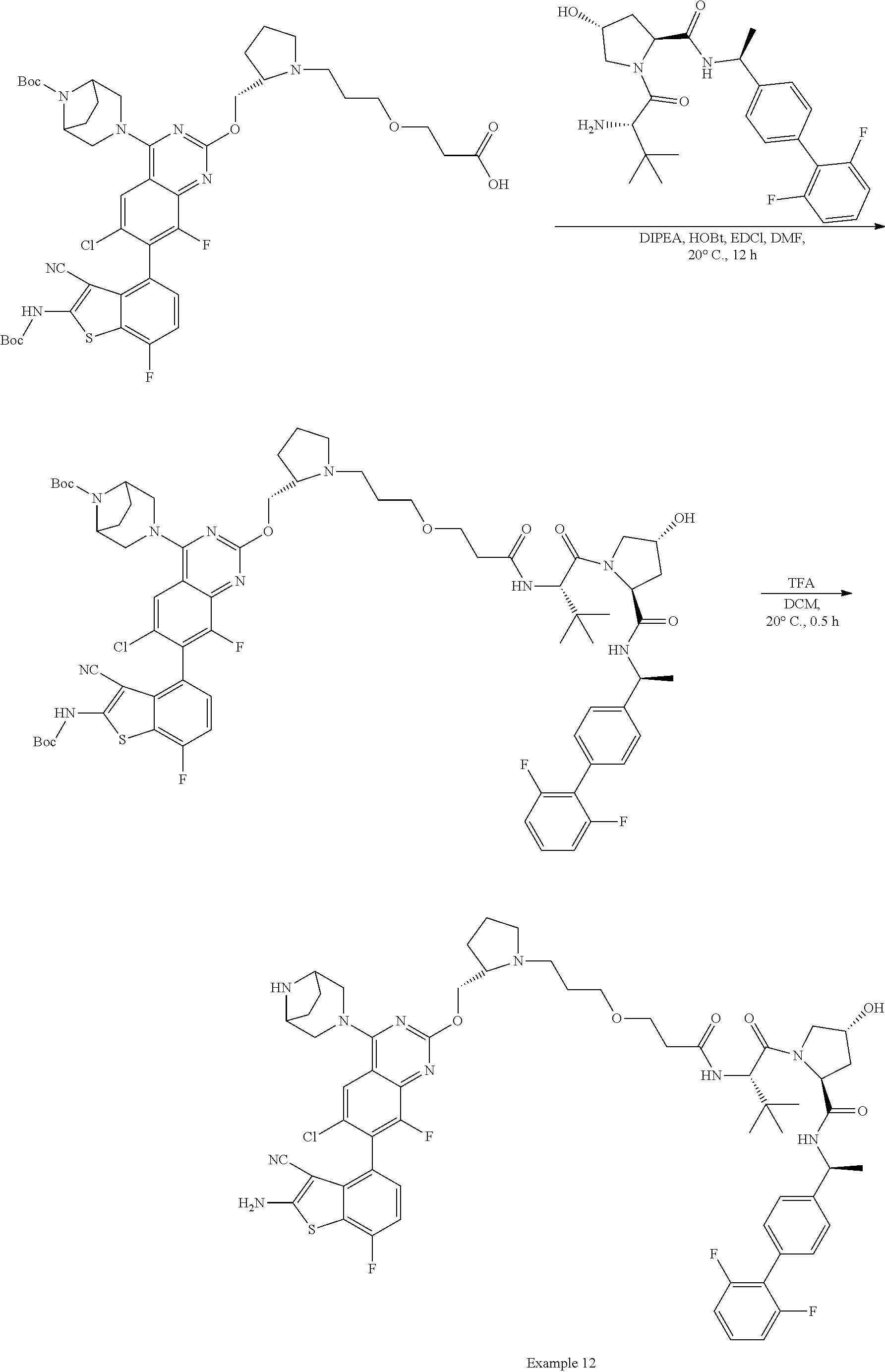

Step 1: tert-butyl 3-[7-[2-(tert-butoxycarbonylamino)-3-cyano-7-fluoro-benzothiophen-4-yl]-6-chloro-2-[[1-[3-[3-[[(1S)-1-[(2S,4R)-2-[[(1S)-1-[4-(2,6-difluorophenyl)phenyl]ethyl]carbamoyl]-4-hydroxy-pyrrolidine-1-carbonyl]-2,2-dimethyl-propyl]amino]-3-oxo-propoxy]propyl]pyrrolidin-2-yl]methoxy]-8-fluoro-quinazolin-4-yl]-3,8-diazabicyclo[3.2.1]octane-8-carboxylate

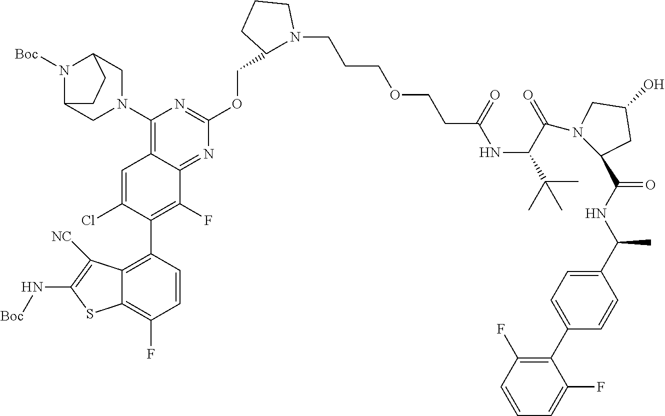

To a solution of (2S,4R)-1-[(2S)-2-amino-3,3-dimethyl-butanoyl]-N-[(1S)-1-[4-(2,6-difluorophenyl)phenyl]ethyl]-4-hydroxy-pyrrolidine-2-carboxamide (80 mg, 0.16 mmol, 1 eq, formate) in N,N-dimethylformamide (5 mL) was added 1-hydroxybenzotriazole (42 mg, 0.32 mmol, 2 eq), N,N-diisopropylethylamine (102 mg, 0.79 mmol, 5 eq), 3-[3-[(2S)-2-[[7-[2-(tert-butoxycarbonylamino)-3-cyano-7-fluoro-benzothiophen-4-yl]-4-(8-tert-butoxycarbonyl-3,8-diazabicyclo[3.2.1]octan-3-yl)-6-chloro-8-fluoro-quinazolin-2-yl]oxymethyl]pyrrolidin-1-yl]propoxy]propanoic acid (173 mg, 0.2 mmol, 1.2 eq) and 1-(3-dimethylaminopropyl)-3-ethylcarbodiimide hydrochloride (76 mg, 0.4 mmol, 2 eq). The mixture was stirred at 20° C. for 12 h. The reaction mixture was partitioned between water (20 mL) and ethyl acetate (20 mL). The organic phase was separated, washed with brine (20 mL×3), dried over anhydrous sodium sulfate, filtered and concentrated under reduced pressure to give a residue. The residue was purified by prep-TLC (SiO2, dichloromethane/methanol=10/1) to get the desired product (138 mg, 0.10 mmol, 64% yield) as a yellow solid. LCMS (ESI, m/z): 1355.10[M+1]⁺.

Step 2: (2S,4R)-1-[(2S)-2-[3-[3-[2-[[7-(2-amino-3-cyano-7-fluoro-benzothiophen-4-yl)-6-chloro-4-(3,8-diazabicyclo[3.2.1]octan-3-yl)-8-fluoro-quinazolin-2-yl]oxymethyl]pyrrolidin-1-yl]propoxy]propanoylamino]-3,3-dimethyl-butanoyl]-N-[(1S)-1-[4-(2,6-difluorophenyl)phenyl]ethyl]-4-hydroxy-pyrrolidine-2-carboxamide

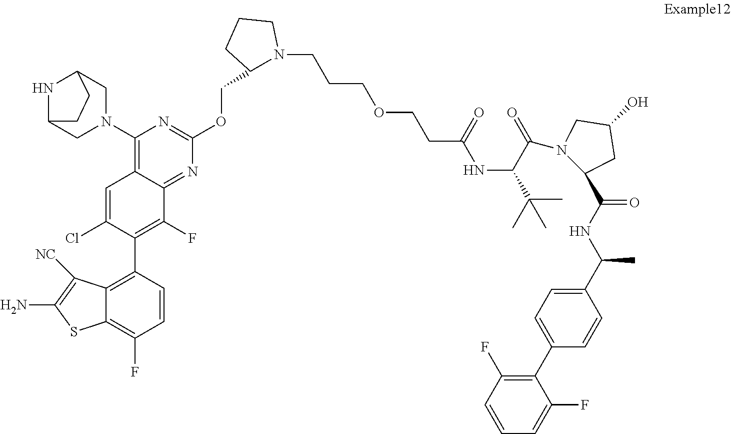

To a solution of tert-butyl 3-[7-[2-(tert-butoxycarbonylamino)-3-cyano-7-fluoro-benzothiophen-4-yl]-6-chloro-2-[[1-[3-[3-[[(1S)-1-[(2S,4R)-2-[[(1S)-1-[4-(2,6-difluorophenyl)phenyl]ethyl]carbamoyl]-4-hydroxy-pyrrolidine-1-carbonyl]-2,2-dimethyl-propyl]amino]-3-oxo-propoxy]propyl]pyrrolidin-2-yl]methoxy]-8-fluoro-quinazolin-4-yl]-3,8-diazabicyclo[3.2.1]octane-8-carboxylate (135 mg, 0.10 mmol, 1 eq) in dichloromethane (3 mL) was added trifluoroacetic acid (11 mg, 0.10 mmol, 1 eq). The mixture was stirred at 20° C. for 0.5 h. The reaction mixture was filtered and concentrated under reduced pressure to give a residue. The residue was purified by prep-HPLC (column: Phenomenex Synergi C18 150*25 mm*10 um; mobile phase: [water(FA)-ACN]; B %: 14%-44%, 10 min) to afford the desired product (52.19 mg, 0.01 mmol, 45% yield) as a yellow solid. LCMS (ESI, m/z): 1154.9 [M+1]⁺. ¹HNMR: (400 MHz, DMSO-d₆) δ: 8.44-8.33 (m, 1H), 8.21 (s, 2H), 8.11 (s, 2H), 7.90-7.77 (m, 2H), 7.52-7.35 (m, 5H), 7.32-7.08 (m, 4H), 4.94 (t, J=7.2 Hz, 1H), 4.52 (d, J=9.6 Hz, 1H), 4.43 (t, J=8.0 Hz, 1H), 4.37-4.25 (m, 4H), 4.09-4.03 (m, 1H), 3.62-3.52 (m, 11H), 3.06-3.03 (m, 1H), 2.93-2.80 (m, 2H), 2.30 (b s, 1H), 2.18 (b d, J=8.4 Hz, 1H), 2.01 (b d, J=0.8, 10.4 Hz, 1H), 1.92-1.80 (m, 2H), 1.74-1.54 (m, 10H), 1.39 (d, J=7.2 Hz, 3H), 0.91 (b d, J=3.2 Hz, 10H)).

Example 13: (2S,4R)-1-[(2S)-2-[3-[3-[(2S)-2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxymethyl]pyrrolidin-1-yl]propoxy]propanoylamino]-3,3-dimethyl-butanoyl]-4-hydroxy-N-[(1S)-1-[4-(4-methylthiazol-5-yl)phenyl]ethyl]pyrrolidine-2-carboxamide

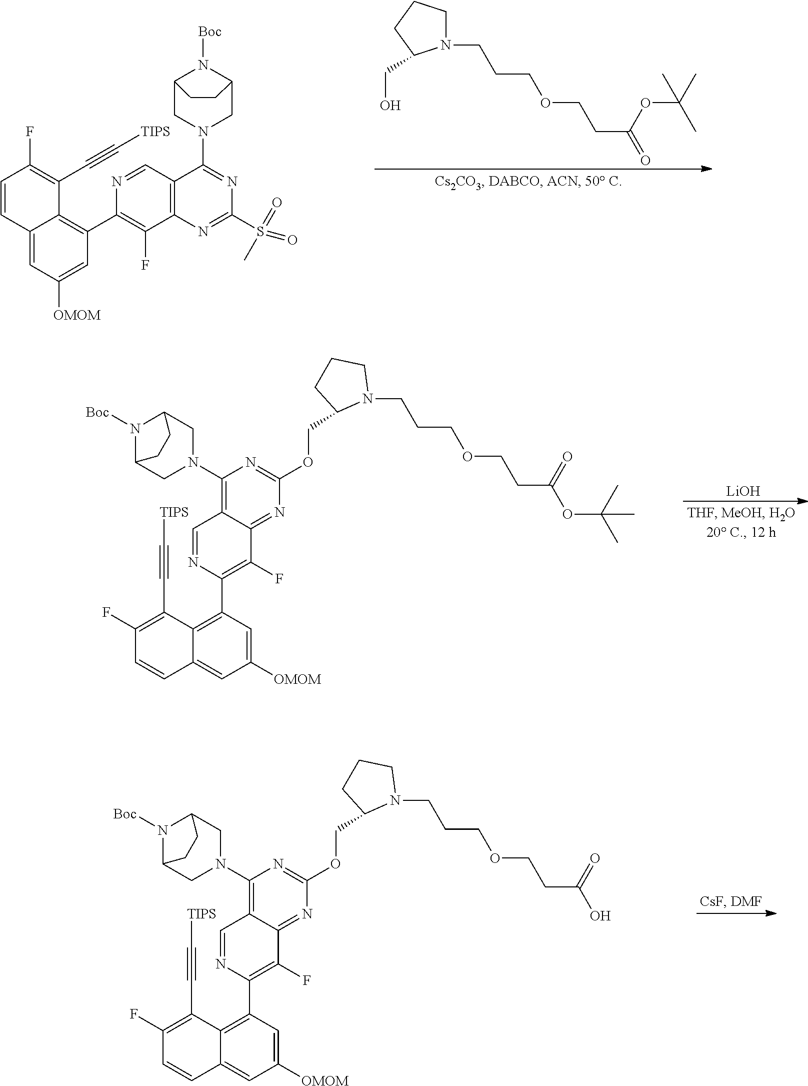

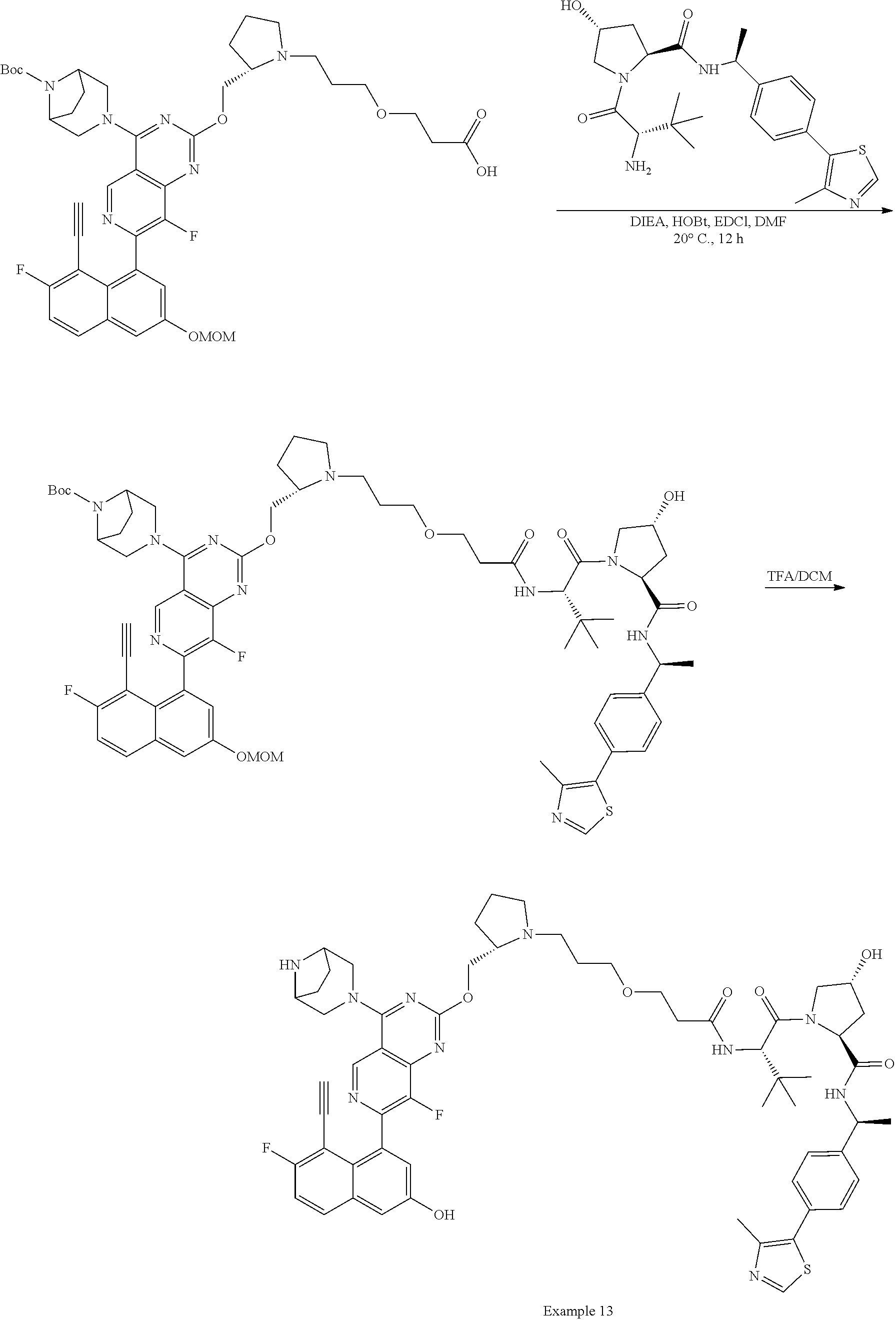

Step 1: tert-butyl 3-[2-[[(2S)-1-[3-(3-tert-butoxy-3-oxo-propoxy)propyl]pyrrolidin-2-yl] methoxy]-8-fluoro-7-[7-fluoro-3-(methoxymethoxy)-8-(2-triisopropylsilylethynyl)-1-naphthyl] pyrido[4,3-d]pyrimidin-4-yl]-3,8-diazabicyclo [3.2.1]octane-8-carboxylate

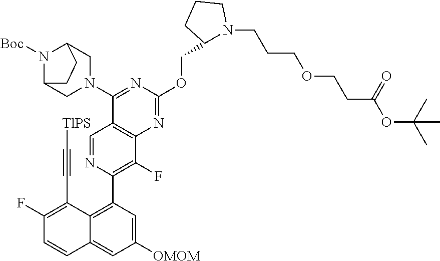

To a solution of tert-butyl 3-[8-fluoro-7-[7-fluoro-3-(methoxymethoxy)-8-(2-triisopropylsilylethynyl)-1-naphthyl]-2-methylsulfonyl-pyrido [4,3-d]pyrimidin-4-yl]-3,8-diazabicyclo[3.2.1]octane-8-carboxylate (3 g, 3.65 mmol, 1 eq), tert-butyl 3-[3-[(2S)-2-(hydroxymethyl)pyrrolidin-1-yl]propoxy]propanoate (1.26 g, 4.38 mmol, 1.2 eq) in acetonitrile (40 mL) was added cesium carbonate (3.57 g, 10.95 mmol, 3 eq) and 1,4-diaza-bicyclo[2.2.2]octane (40 mg, 0.36 mmol, 0.1 eq). The mixture was stirred at 50° C. for 1 h. The reaction mixture was filtered and concentrated in reduced pressure to give a residue. The residue was purified by prep-HPLC (column: Phenomenex Synergi Max-RP 250*50 mm*10 um; mobile phase: [water(FA)-ACN]; B %: 50%-80%, 20 min) to afford the desired product (2.3 g, 2.23 mmol, 61% yield) as a colorless oil. LCMS (ESI, m/z): 1029.5 [M]⁺.

Step 2: 3-[3-[(2S)-2-[[4-(8-tert-butoxycarbonyl-3,8-diazabicyclo[3.2.1]octan-3-yl)-8-fluoro-7-[7-fluoro-3-(methoxymethoxy)-8-(2-triisopropylsilylethynyl)-1-naphthyl]pyrido[4,3-d]pyrimidin-2-yl]oxymethyl]pyrrolidin-1-yl]propoxy]propanoic acid

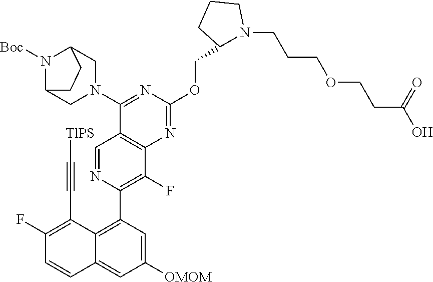

To a solution of tert-butyl 3-[2-[[(2S)-1-[3-(3-tert-butoxy-3-oxo-propoxy)propyl]pyrrolidin-2-yl]methoxy]-8-fluoro-7-[7-fluoro-3-(methoxymethoxy)-8-(2-triisopropylsilylethynyl)-1-naphthyl]pyrido[4,3-d]pyrimidin-4-yl]-3,8-diazabicyclo[3.2.1]octane-8-carboxylate (800 mg, 0.77 mmol, 1 eq) in methanol (4 mL), tetrahydrofuran (4 mL), water (2 mL) was added lithium hydroxide (326 mg, 7.77 mmol, 10 eq). The mixture was stirred at 25° C. for 1 h. The reaction mixture was adjusted the pH to 5-6 by adding aqueous hydrochloric acid (1M), then the mixture was extracted with ethyl acetate (20 mL×3). The combined organic phase was washed with brine (30 mL×3), dried with anhydrous sodium sulfate, filtered and concentrated in vacuum. The residue was purified by prep-TLC (Dichloromethane/Methanol=8/1) to afford the desired product (130 mg, 0.13 mmol, 17% yield) as a white solid. LCMS (ESI, m/z): 973.5[M]⁺.

Step 3: 3-[3-[(2S)-2-[[4-(8-tert-butoxycarbonyl-3,8-diazabicyclo[3.2.1]octan-3-yl)-7-[8-ethynyl-7-fluoro-3-(methoxymethoxy)-1-naphthyl]-8-fluoro-pyrido[4,3-d]pyrimidin-2-yl]oxymethyl]pyrrolidin-1-yl]propoxy]propanoic acid

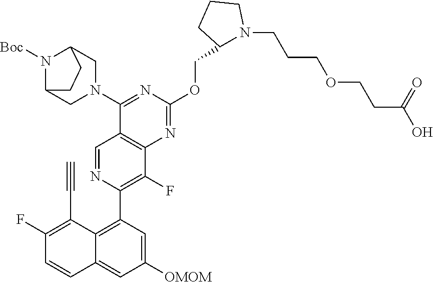

To a solution of 3-[3-[(2S)-2-[[4-(8-tert-butoxycarbonyl-3,8-diazabicyclo[3.2.1]octan-3-yl)-8-fluoro-7-[7-fluoro-3-(methoxymethoxy)-8-(2-triisopropylsilylethynyl)-1-naphthyl]pyrido[4,3-d]pyrimidin-2-yl]oxymethyl]pyrrolidin-1-yl]propoxy]propanoic acid (130 mg, 0.13 mmol, 1 eq) in N,N-dimethylformamide (2 mL) was added cesium fluoride (304 mg, 2.00 mmol, 15 eq). The mixture was stirred at 25° C. for 0.5 h. The reaction mixture was filtered and extracted with ethyl acetate (20 mL×3). The combined organic phase was washed with brine (20 mL×2), dried with anhydrous sodium sulfate, filtered and concentrated in vacuum. The crude product (100 mg, 0.12 mmol, 91% yield) was obtained as a yellow solid and was used for the next step reaction without purification. LCMS (ESI, m/z): 817.4[M+1]⁺.

Step 4: tert-butyl 3-[7-[8-ethynyl-7-fluoro-3-(methoxymethoxy)-1-naphthyl]-8-fluoro-2-[[(2S)-1-[3-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl] amino]-3-oxo-propoxy]propyl]pyrrolidin-2-yl]methoxy]pyrido[4,3-d]pyrimidin-4-yl]-3,8-diazabicyclo[3.2.1]octane-8-carboxylate

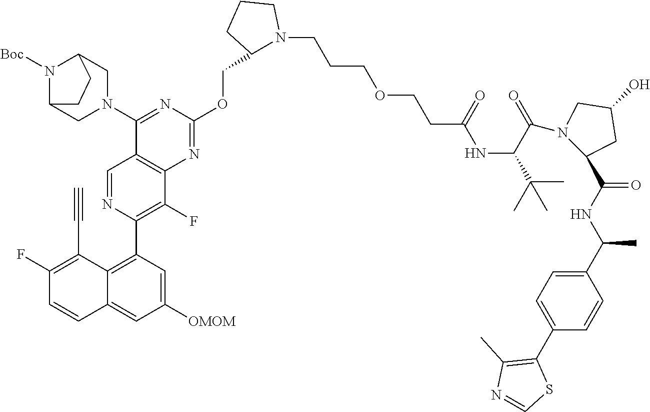

To a solution of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (58 mg, 0.12 mmol, 1 eq, hydrochloride) in N,N-dimethylformamide (2 mL) was added N,N-diisopropylethylamine (47 mg, 0.36 mmol, 3 eq), 3-[3-[(2S)-2-[[4-(8-tert-butoxycarbonyl-3,8-diazabicyclo[3.2.1]octan-3-yl)-7-[8-ethynyl-7-fluoro-3-(methoxymethoxy)-1-naphthyl]-8-fluoro-pyrido[4,3-d]pyrimidin-2-yl]oxymethyl]pyrrolidin-1-yl]propoxy]propanoic acid (100 mg, 0.12 mmol, 1 eq), 1-(3-dimethylaminopropyl)-3-ethylcarbodiimide (70 mg, 0.36 mmol, 3 eq) and 1-hydroxybenzotriazole (49 mg, 0.36 mmol, 3 eq). The mixture was stirred at 25° C. for 1 h. The reaction mixture was extracted with ethyl acetate (30 mL×3). The combined organic phase was washed with brine (20 mL×2), dried with anhydrous sodium sulfate, filtered and concentrated in vacuum. The residue was purified by prep-TLC (Dichloromethane/Methanol=8/1) to afford the desired product (80 mg, 0.064 mmol, 52% yield) as a yellow oil.

Step 5: (2S,4R)-1-[(2S)-2-[3-[3-[(2S)-2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxymethyl]pyrrolidin-1-yl]propoxy]propanoylamino]-3,3-dimethyl-butanoyl]-4-hydroxy-N-[(1S)-1-[4-(4-methylthiazol-5-yl)phenyl]ethyl]pyrrolidine-2-carboxamide

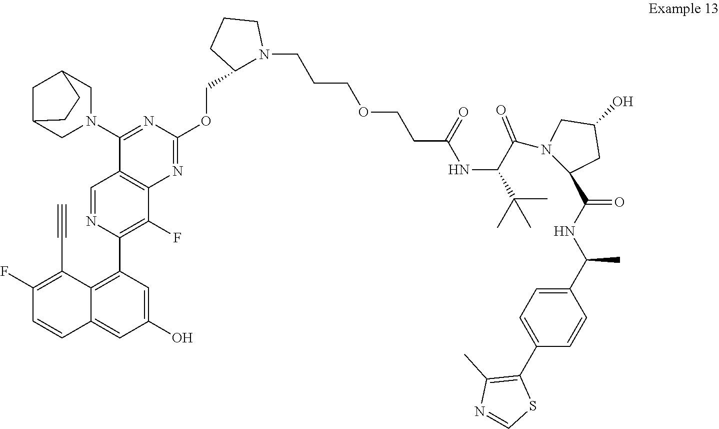 [SMILES: C#Cc1c(F)ccc2cc(O)cc(-c3ncc4c(N5CC6CCC(C6)C5)nc(OC[C@@H]5CCCN5CCCOCCC(=O)N[C@H](C(=O)N5C[C@H](O)C[C@H]5C(=O)N[C@@H](C)c5ccc(-c6scnc6C)cc5)C(C)(C)C)nc4c3F)c12]

To a solution of tert-butyl 3-[7-[8-ethynyl-7-fluoro-3-(methoxymethoxy)-1-naphthyl]-8-fluoro-2-[[(2S)-1-[3-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]propyl] pyrrolidin-2-yl]methoxy]pyrido[4,3-d]pyrimidin-4-yl]-3,8-diazabicyclo[3.2.1]octane-8-carboxylate (40 mg, 0.032 mmol, 1 eq) in dichloromethane (1 mL) was added trifluoroacetic acid (770.00 mg, 6.75 mmol, 0.5 mL, 209 eq). The mixture was stirred at 25° C. for 20 min. The reaction mixture was concentrated in reduced pressure to give a residue. The residue was purified by prep-HPLC (column: Phenomenex luna C18 150*25 mm*10 um; mobile phase: [water(TFA)-ACN]; B %: 15%-45%, 1 1 min), then the eluent was extracted with dichloromethane (50 mL×3). The combined organic phase was washed with brine (20 mL×2), dried with anhydrous sodium sulfate, filtered and concentrated in vacuum to afford the desired product (15 mg, 0.013 mmol, 42% yield, 100% purity) as a yellow solid. LCMS (ESI, m/z): 1098.7[M]+. ¹H NMR: (400 MHz, DMSO-d₆) δ: 10.15 (br s, 1H), 9.04 (s, 1H), 8.99 (s, 1H), 8.37 (br d, J=7.6 Hz, 1H), 7.98 (dd, J=6.4, 9.2 Hz, 1H), 7.82 (dd, J=2.4, 9.2 Hz, 1H), 7.51-7.27 (m, 6H), 7.18 (d, J=2.4 Hz, 1H), 5.20-5.05 (m, 1H), 4.91 (br t, J =7.2 Hz, 1H), 4.52-4.26 (m, 5H), 4.12-4.02 (m, 1H), 3.93 (d, J=3.2 Hz, 1H), 3.78-3.44 (m, 8H), 3.39 (s, 2H), 3.09-3.00 (m, 1H), 2.93-2.73 (m, 2H), 2.46 (s, 3H), 2.23-2.11 (m, 2H), 2.05-1.97 (m, 2H), 1.67 (br s, 4H), 1.37 (br d, J=7.2 Hz, 3H), 1.16 (br d, J=6.4 Hz, 6H), 0.90 (br d, J=2.4 Hz, 9H), 0.84-0.77 (m, 4H).

Example 14: (2S,4R)-1-[(2S)-2-[3-[3-[(2S)-2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxymethyl]pyrrolidin-1-yl]propoxy]propanoylamino]-3,3-dimethyl-butanoyl]-N-[(1S)-1-[4-(2,6-difluorophenyl)phenyl]ethyl]-4-hydroxy-pyrrolidine-2-carboxamide

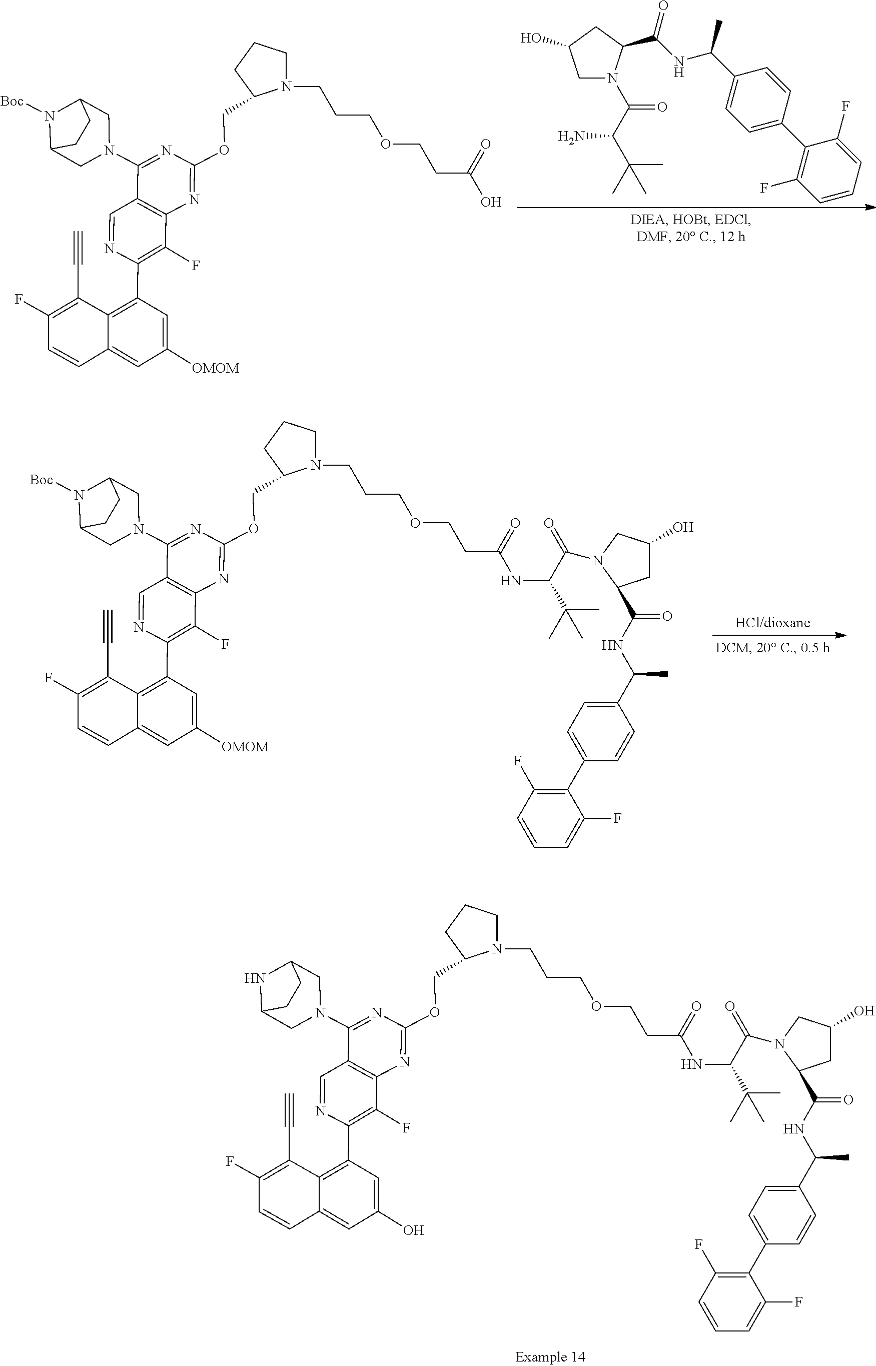

Step 1: tert-butyl 3-[2-[[(2S)-1-[3-[3-[[(1S)-1-[(2S,4R)-2-[[(1S)-1-[4-(2,6-difluorophenyl)phenyl]ethyl]carbamoyl]-4-hydroxy-pyrrolidine-1-carbonyl]-2,2-dimethyl-propyl]amino]-3-oxo-propoxy]propyl]pyrrolidin-2-yl]methoxy]-7-[8-ethynyl-7-fluoro-3-(methoxymethoxy)-1-naphthyl]-8-fluoro-pyrido[4,3-d]pyrimidin-4-yl]-3,8-diazabicyclo [3.2.1]octane-8-carboxylate

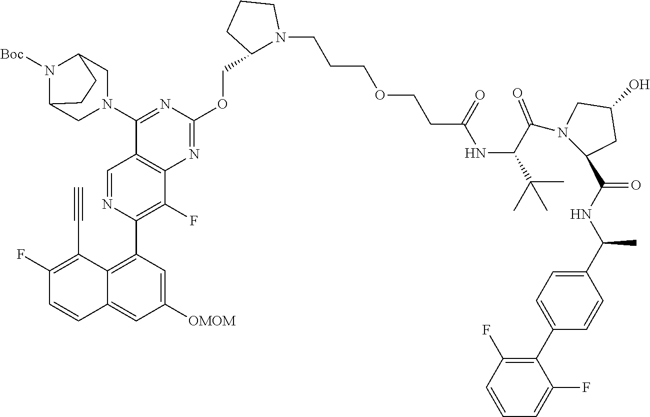

To a solution of (2S,4R)-1-[(2S)-2-amino-3,3-dimethyl-butanoyl]-N-[(1S)-1-[4-(2,6-difluorophenyl)phenyl]ethyl]-4-hydroxy-pyrrolidine-2-carboxamide (56 mg, 0.12 mmol, 1 eq), 3-[3-[(2S)-2-[[4-(8-tert-butoxycarbonyl-3,8-diazabicyclo[3.2.1]octan-3-yl)-7-[8-ethynyl-7-fluoro-3-(methoxymethoxy)-1-naphthyl]-8-fluoro-pyrido[4,3-d]pyrimidin-2-yl]oxymethyl] pyrrolidin-1-yl]propoxy]propanoic acid (100 mg, 0.12 mmol, 1 eq) in N,N-dimethylformamide (3 mL) was added N,N-diisopropylethylamine (47 mg, 0.36 mmol, 3 eq), 1-hydroxybenzotriazole (33 mg, 0.24 mmol, 2 eq) and N-(3-dimethylaminopropyl)-N-ethylcarbodiimide hydrochloride (47 mg, 0.24 mmol, 2 eq). The mixture was stirred at 20° C. for 12 hours. The reaction mixture was diluted with water (10 mL) and extracted with ethyl acetate (10 mL×3). The combined organic layers were washed with brine (10 mL×3), dried over anhydrous sodium sulfate, filtered and concentrated under reduced pressure to give a residue which was purified by prep-TLC (silicon dioxide, dichloromethane/methanol=20/1) to give the desired product (61 mg, 45 mmol, 37% yield, 94% purity) as a yellow solid. LCMS (ESI, m/z): 1258.6[M]⁺.

Step 2: (2S,4R)-1-[(2S)-2-[3-[3-[(2S)-2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxymethyl]pyrrolidin-1-yl]propoxy]propanoylamino]-3,3-dimethyl-butanoyl]-N-[(1 S)-1-[4-(2,6-difluorophenyl)phenyl]ethyl]-4-hydroxy-pyrrolidine-2-carboxamide

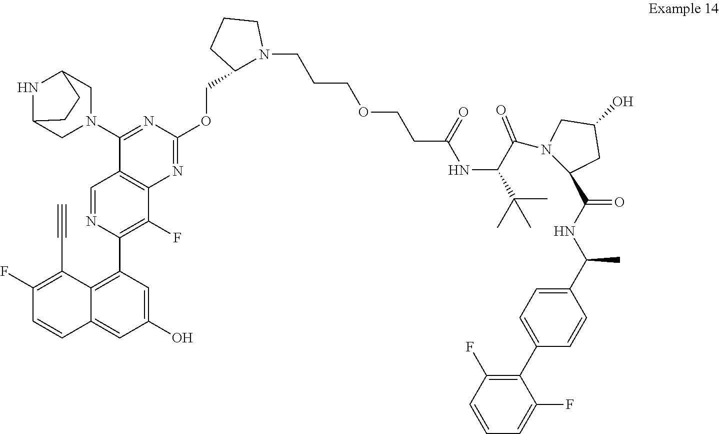 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(F)cccc6F)cc5)C(C)(C)C)nc4c3F)c12]

To a solution of tert-butyl 3-[2-[[(2S)-1-[3-[3-[[(1S)-1-[(2S,4R)-2-[[(1S)-1-[4-(2,6-difluorophenyl)phenyl]ethyl]carbamoyl]-4-hydroxy-pyrrolidine-1-carbonyl]-2,2-dimethyl-propyl]amino]-3-oxo-propoxy]propyl]pyrrolidin-2-yl]methoxy]-7-[8-ethynyl-7-fluoro-3-(methoxymethoxy)-1-naphthyl]-8-fluoro-pyrido[4,3-d]pyrimidin-4-yl]-3,8-diazabicyclo[3.2.1]octane-8-carboxylate (61 mg, 0.05 mmol, 1 eq) in dichloromethane (3 mL) was added trifluoroacetic acid (308 mg, 2.7 mmol, 56 eq). The mixture was stirred at 20° C. for 0.5 hour. The reaction mixture was bubbled by nitrogen gas to remove dichloromethane. The residue was purified by prep-HPLC (column: 3_Phenomenex Luna C18 75*30 mm*3 um; mobile phase: [water(trifluoroacetic acid)-acetonitrile]; B %: 29%-49%, 9 min), then added sodium hydroxide and extracted with ethyl acetate (10 mL×3). The combined organic layers were washed with brine (10 mL×2), dried over anhydrous sodium sulfate, filtered and concentrated under reduced pressure to give the desired product (27 mg, 0.02 mmol, 50% yield, 99% purity) as a yellow solid. LCMS (ESI, m/z): 558.0 [M/2+1]+. ¹H NMR: (400 MHz, DMSO-d₆) δ: 9.37 (s, 1H), 8.23 (s, 1H), 7.56 (d, J=8.0 Hz, 1H), 7.19-7.11 (m, 1H), 7.00 (d, J=9.6 Hz, 1H), 6.67-6.61 (m, 2H), 6.57 (s, 5H), 6.42-6.35 (m, 3H), 4.17-4.06 (m, 1H), 3.69 (s, 2H), 3.63-3.50 (m, 3H), 3.46 (s, 1H), 3.32-3.22 (m, 1H), 3.11 (d, J=2.8 Hz, 1H), 2.87 (s, 2H), 2.82 (d, J=12.4 Hz, 2H), 2.77-2.67 (m, 4H), 2.56 (d, J=6.4 Hz, 2H), 2.23 (dd, J=3.6, 5.2 Hz, 1H), 2.00 (d, J=2.8 Hz, 2H), 1.48-1.43 (m, 1H), 1.38 (d, J=4.8 Hz, 1H), 1.22-1.13 (m, 2H), 1.11-1.07 (m, 1H), 0.94-0.85 (m, 8H), 0.65 (d, J=6.1 Hz, 1H), 0.56 (d, J=6.8 Hz, 3H), 0.42 (s, 3H), 0.08 (d, J=2.4 Hz, 9H).

Example 15

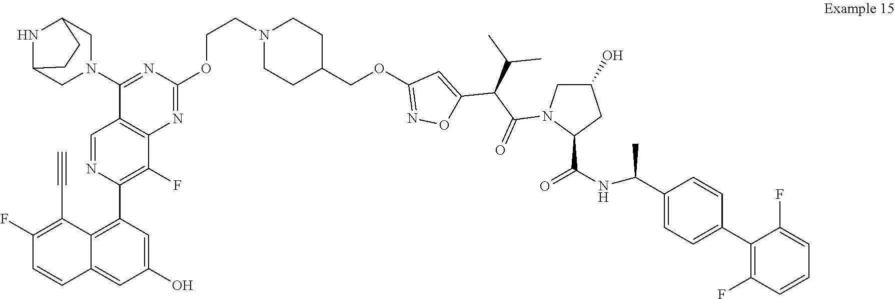 [SMILES: C#Cc1c(F)ccc2cc(O)cc(-c3ncc4c(N5CC6CCC(C5)N6)nc(OCCN5CCC(COc6cc([C@H](C(=O)N7C[C@H](O)C[C@H]7C(=O)N[C@@H](C)c7ccc(-c8c(F)cccc8F)cc7)C(C)C)on6)CC5)nc4c3F)c12]

Synthesis of Intermediate 9: tert-butyl 4-(((5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy)methyl)piperidine-1-carboxylate

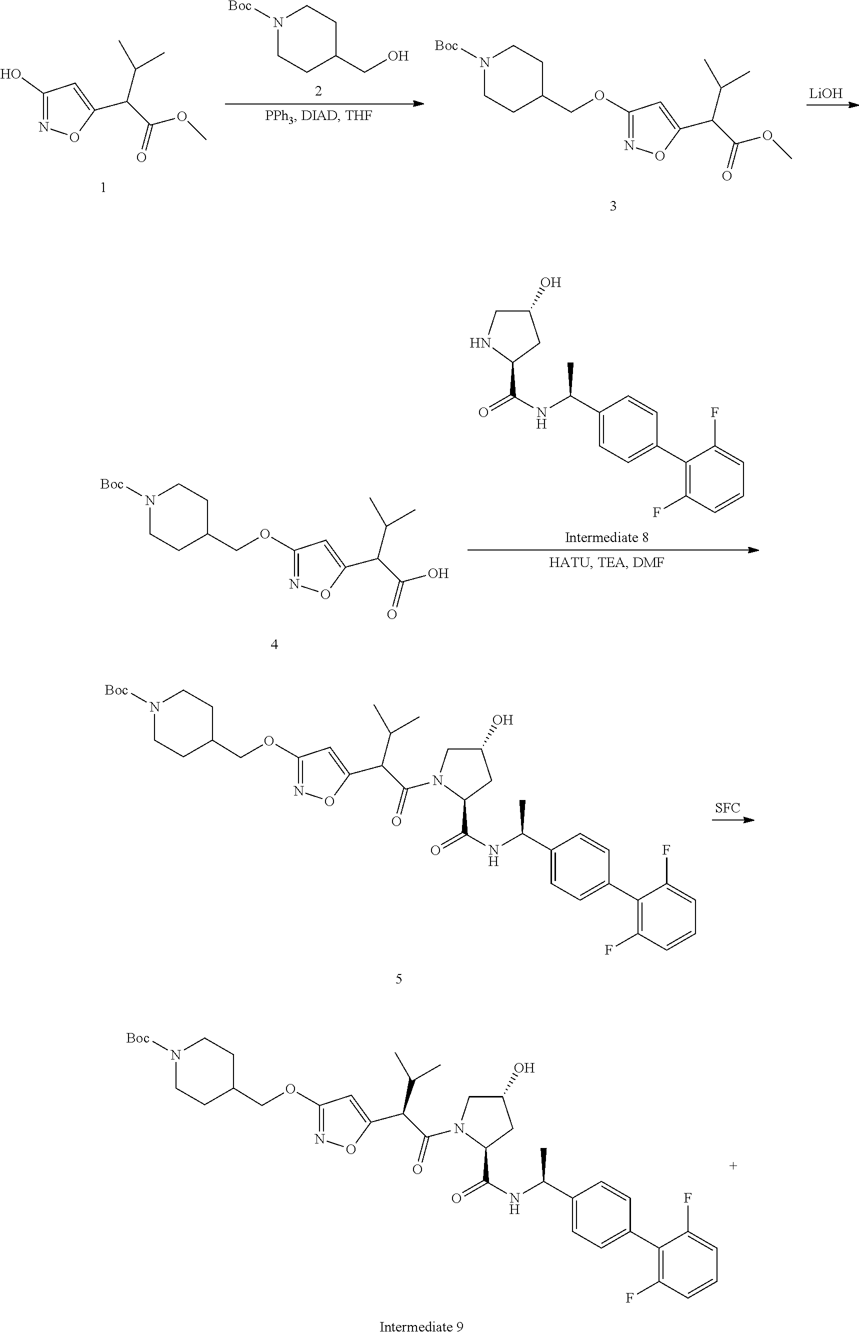

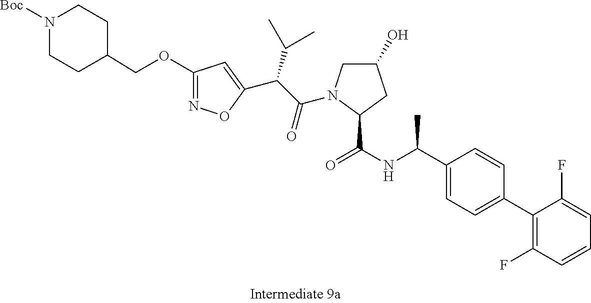 [SMILES: CC(C)[C@H](C(=O)N1C[C@H](O)C[C@H]1C(=O)N[C@@H](C)c1ccc(-c2c(F)cccc2F)cc1)c1cc(OCC2CCN(C(=O)OC(C)(C)C)CC2)no1]

Compound 1 is reacted with compound 2 following Mitsunobu reaction conditions to afford compound 3. Compound 3 is treated with LiOH to give compound 4. Compound 4 and Intermediate 8 are reacted under amide coupling conditions to afford compound 5. The diasteromers of compound 5 are separated by SFC to give Intermediate 9 and its diasteromer Intermediate 9a.

Synthesis of Intermediate 10: tert-butyl 3-(7-(8-ethynyl-7-fluoro-3-hydroxynaphthalen-1-yl)-8-fluoro-2-(2-oxoethoxy)pyrido[4,3-d]pyrimidin-4-yl)-3,8-diazabicyclo[3.2.1]octane-8-carboxylate

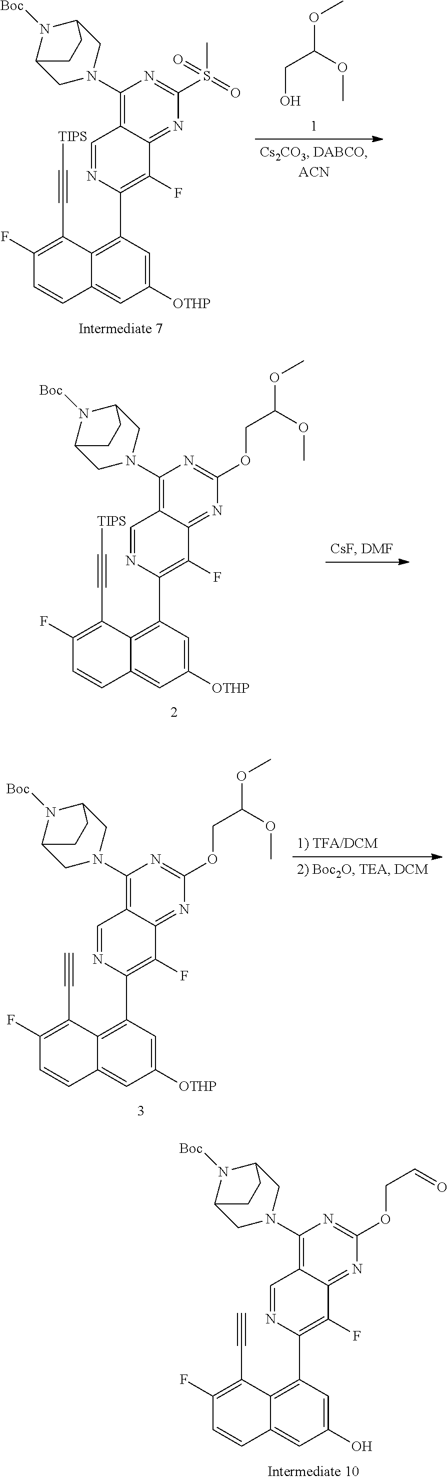

Intermediate 7 is reacted with compound 1 to yield compound 2. After treatment with CsF, compound 2 is converted to compound 3. Compound 3 is first reacted with TFA, and the resultant product is then treated with Boc₂O to give Intermediate 10.

Synthesis of Example 15: (2S,4R)-1-((2R)-2-(3-((1-(2-((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)ethyl)piperidin-4-yl)methoxy)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide

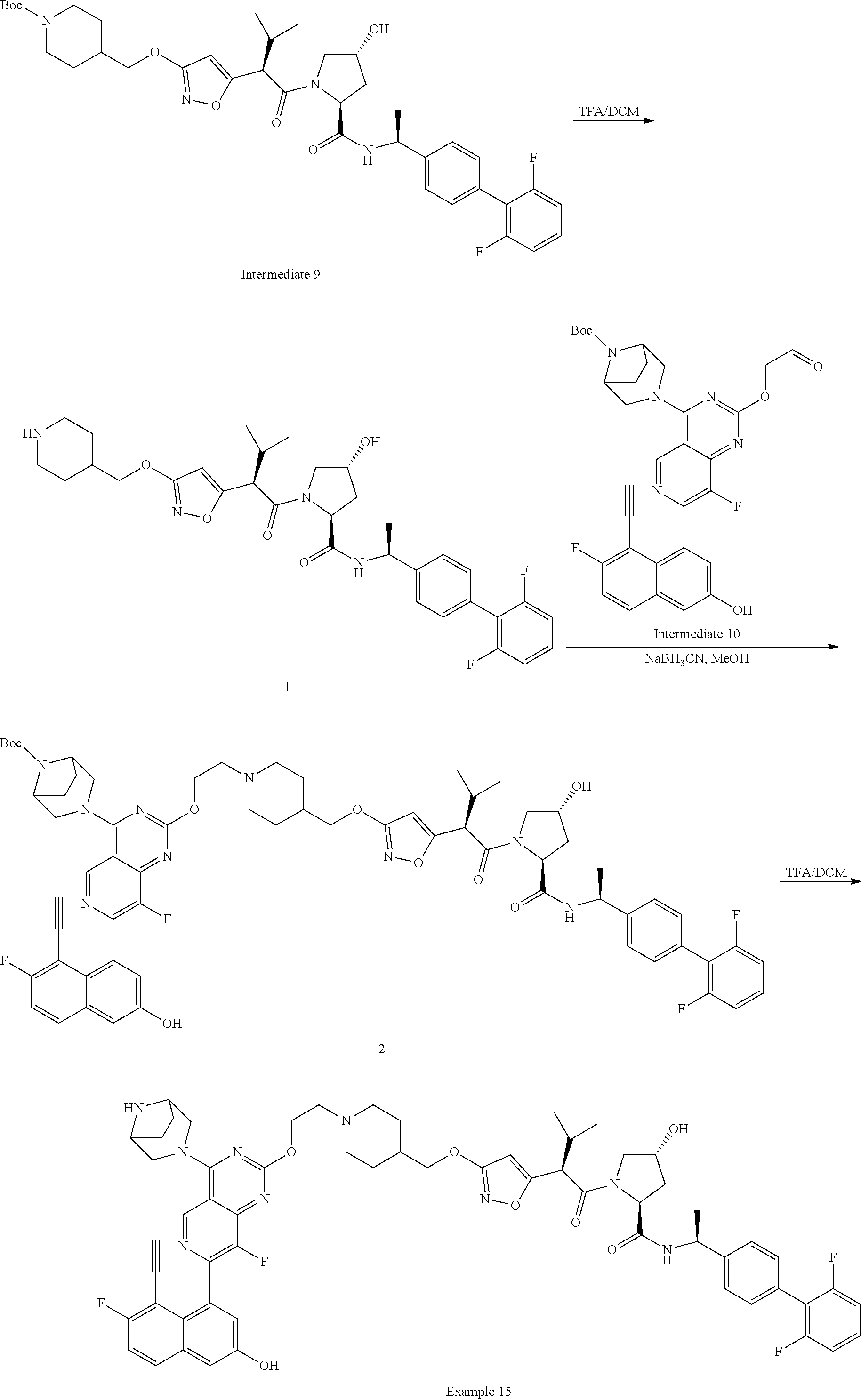

Intermediate 9 is converted to compound 1 after treatment with TFA. Compound 1 is reacted with Intermediate 10 under reductive amination conditions to afford compound 2. Compound 2 is reacted with TFA to generate Example 15.

Example 15: (2S,4R)-1-((2R)-2-(3-((1-(2-((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)ethyl)piperidin-4-yl)methoxy)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide

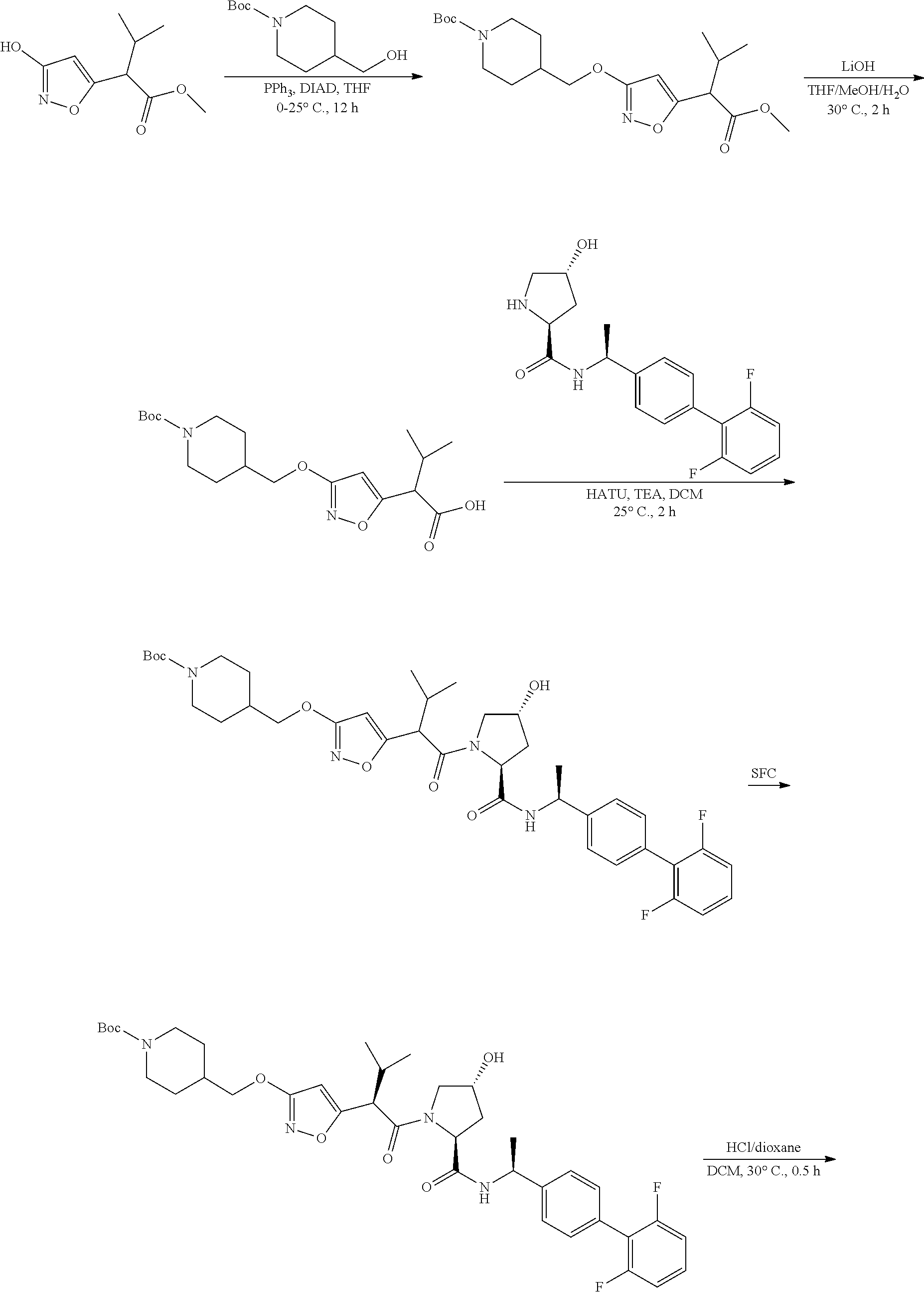

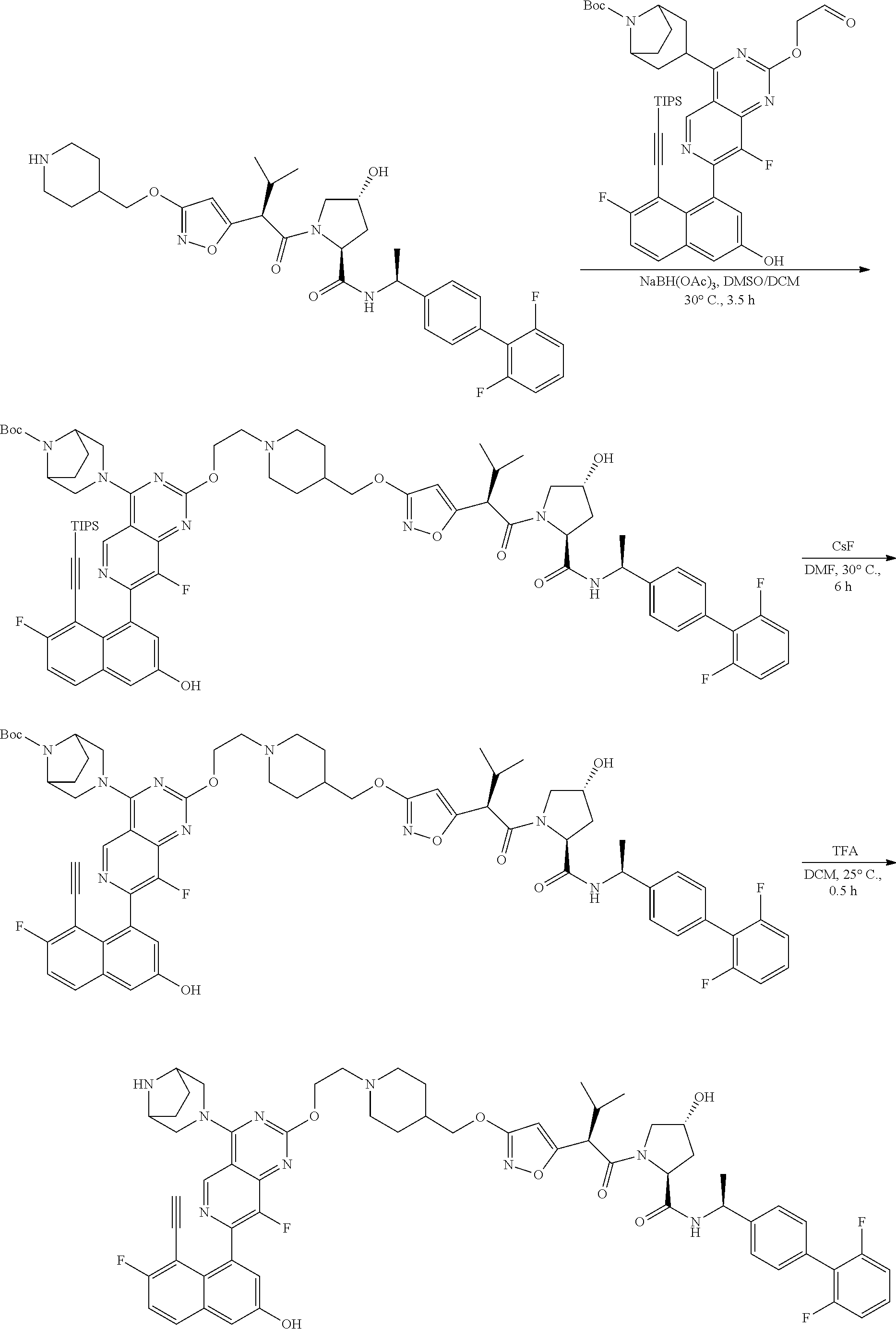

Step 1: tert-butyl 4-(((5-(1-methoxy-3-methyl-1-oxobutan-2-yl)isoxazol-3-yl)oxy)methyl)piperidine-1-carboxylate

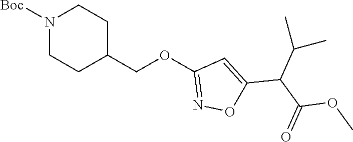 [SMILES: COC(=O)C(c1cc(OCC2CCN(C(=O)OC(C)(C)C)CC2)no1)C(C)C]

To a solution of methyl 2-(3-hydroxyisoxazol-5-yl)-3-methyl-butanoate (2 g, 10.04 mmol, 1.00 eq) and tert-butyl 4-(hydroxymethyl)piperidine-1-carboxylate (2.16 g, 10.04 mmol, 1.00 eq) in tetrahydrofuran (20 mL) was added triphenylphosphine (5.27 g, 20.08 mmol, 2.00 eq) and diisopropyl azodicarboxylate (3.05 g, 15.06 mmol, 1.50 eq) at 0° C. in nitrogen. The mixture was stirred at 25° C. for 12 h. LCMS showed that the desired product was detected. The reaction mixture was concentrated under reduced pressure to get a residue. The residue was purified by preparative high performance liquid chromatography (column: Welch Ultimate XB-CN 250*70*10 um; mobilephase: [Hexane-EtOH (0.1% NH3·H2O)]; B %: 1%-15%, 15 min) to afford the desired product (2.2 g, 5.55 mmol, 55% yield) as a yellow oil. LCMS (ESI, m/z): 397.1 [M+H]⁺. ¹H NMR (400 MHz, CDCl₃) δ 5.81 (s, 1H), 4.24-4.05 (m, 2H), 4.00 (d, J=6.4 Hz, 2H), 3.77-3.60 (m, 3H), 3.45-3.38 (m, 1H), 2.66 (t, J=11.6 Hz, 2H), 2.39-2.16 (m, 1H), 1.95-1.83 (m, 1H), 1.69 (d, J=12.4 Hz, 2H), 1.39 (s, 9H), 1.22-1.16 (m, 2H), 0.96-0.84 (m, 6H).

Step 2: 2-(3-((1-(tert-butoxycarbonyl)piperidin-4-yl)methoxy)isoxazol-5-yl)-3-methylbutanoic acid

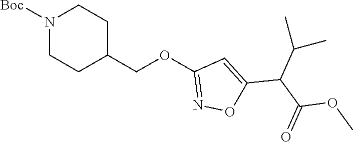 [SMILES: COC(=O)C(c1cc(OCC2CCN(C(=O)OC(C)(C)C)CC2)no1)C(C)C]

To a solution of tert-butyl 4-(((5-(1-methoxy-3-methyl-1-oxobutan-2-yl)isoxazol-3-yl)oxy)methyl)piperidine-1-carboxylate (2.20 g, 5.55 mmol, 1.00 eq) in the mixed solvent of tetrahydrofuran (10 mL), methanol (10 mL) and water (10 mL) was added lithium hydroxide (1.16 g, 27.74 mmol, 5.00 eq). The mixture was stirred at 30° C. for 2 h. TLC (petroleum ether:ethyl acetate=5:1) indicated the reactant was consumed and one new spot was formed. The reaction mixture was concentrated under reduced pressure to get a yellow oil. pH of the reaction mixture was adjusted to 4 by hydrochloric acid (1 M), then extracted with dichloromethane (50 mL×3). The combined organic layers were washed with brine (50 mL), dried over anhydrous sodium sulfate, filtered and concentrated under reduced pressure to afford the crude product (2.10 g, 5.49 mmol, 99% yield) as a yellow oil, which was used directly in the next step directly. ¹H NMR (400 MHz, DMSO-d₆) δ 13.68 (s, 1H), 6.13 (s, 1H), 4.01 (d, J=6.4 Hz, 2H), 3.96 (d, J=12.0 Hz, 2H), 3.48 (d, J=8.8 Hz, 1H), 2.81-2.65 (m, 2H), 2.30-2.21 (m, 1H), 1.97-1.91 (m, 1H), 1.69 (d, J=11.2 Hz, 2H), 1.39 (s, 9H), 1.19-1.08 (m, 2H), 0.95 (d, J=6.8 Hz, 3H), 0.83 (d, J=6.8 Hz, 3H).

Step 3: tert-butyl 4-(((5-(1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy)methyl)piperidine-1-carboxylate

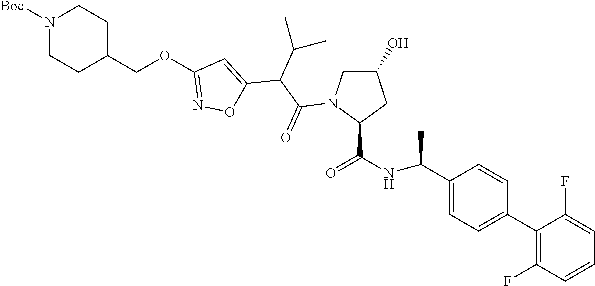 [SMILES: CC(C)C(C(=O)N1C[C@H](O)C[C@H]1C(=O)N[C@@H](C)c1ccc(-c2c(F)cccc2F)cc1)c1cc(OCC2CCN(C(=O)OC(C)(C)C)CC2)no1]

To a solution of (2S,4R)—N-[(1S)-1-[4-(2,6-difluorophenyl)phenyl]ethyl]-4-hydroxy-pyrrolidine-2-carboxamide (500 mg, 1.09 mmol, 1.00 eq, trifluoroacetate) in dichloromethane (10 mL), was added diisopropylethylamine (421 mg, 3.26 mmol, 3.00 eq), 2-(3-((1-(tert-butoxycarbonyl)piperidin-4-yl)methoxy)isoxazol-5-yl)-3-methylbutanoic acid (415 mg, 1.09 mmol, 1.00 eq) and o-(7-Azabenzotriazol-1-yl)-N,N,N′,N′-tetramethyluronium hexafluorophosphate (619 mg, 1.63 mmol, 1.50 eq). The mixture was stirred at 25° C. for 1 h. LCMS showed that the desired mass was detected. Dichloromethane (20 mL) and water (30 mL) were added before the mixture was extracted with dichloromethane (30 mL×3). The combined organic layers were washed with brine (30 mL), dried over anhydrous sodium sulfate, filtered and concentrated under reduced pressure to get the residue. The residue was purified by preparative high performance liquid chromatography (column: Phenomenex Luna C8 250*50 mm*10 um; mobile phase: [water(FA)-ACN]; B %: 45%-75%, 20 min) to afford the product (500 mg, 0.70 mmol, 64% yield) as a yellow solid. LCMS (ESI, m/z): 711.1 [M+H]⁺.

Step 4: tert-butyl 4-(((5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy)methyl)piperidine-1-carboxylate

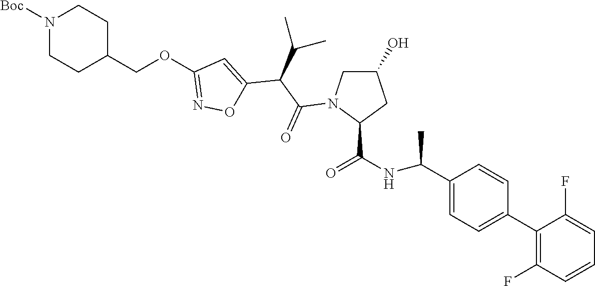 [SMILES: CC(C)[C@@H](C(=O)N1C[C@H](O)C[C@H]1C(=O)N[C@@H](C)c1ccc(-c2c(F)cccc2F)cc1)c1cc(OCC2CCN(C(=O)OC(C)(C)C)CC2)no1]

The mixture of diastereoisomers tert-butyl 4-(((5-(1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy)methyl)piperidine-1-carboxylate (500 mg, 0.70 mmol, 1.00 eq) was separated by SFC(column: DAICEL CHIRALPAK AD(250 mm*30 mm, 10 um); mobile phase: [0.1% NH3H2O ETOH]; B %: 30%-30%, 7 min), and the second eluent (t_{R}=1.674 min) was identified as the desired diastereoisomer tert-butyl 4-(((5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy)methyl)piperidine-1-carboxylate. The product (240 mg, 0.32 mmol, 45% yield, 94.9% purity) was obtained as a white solid.

Step 5: (2S,4R)—N—((S)-1-(2′,6′-difluoro-[1,1′-biphenyl]-4-yl)ethyl)-4-hydroxy-1-((R)-3-methyl-2-(3-(piperidin-4-ylmethoxy)isoxazol-5-yl)butanoyl)pyrrolidine-2-carboxamide

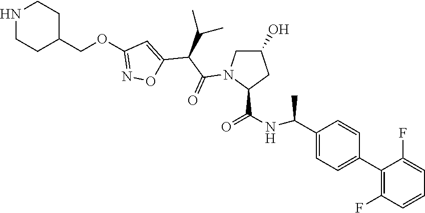 [SMILES: CC(C)[C@@H](C(=O)N1C[C@H](O)C[C@H]1C(=O)N[C@@H](C)c1ccc(-c2c(F)cccc2F)cc1)c1cc(OCC2CCNCC2)no1]

To a solution of tert-butyl 4-(((5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy)methyl)piperidine-1-carboxylate (120 mg, 0.168 mmol, 1.00 eq) in dichloromethane (2 mL), was added hydrogen chloride/dioxane (4 M, 2 mL, 47.39 eq). The mixture was stirred at 30° C. for 0.5 h. TLC (dichloromethane:methanol=10:1) showed that the reaction was completed. The reaction mixture was concentrated under reduced pressure to afford the crude product (109 mg, 0.168 mmol, 99% yield, hydrochloride) as a white solid, which was used in the next step directly.

Step 6: tert-butyl 3-(2-(2-(4-(((5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy)methyl)piperidin-1-yl)ethoxy)-8-fluoro-7-(7-fluoro-3-hydroxy-8-((triisopropylsilyl)ethynyl)naphthalen-1-yl)pyrido[4,3-d]pyrimidin-4-yl)-3,8-diazabicyclo[3.2.1]octane-8-carboxylate

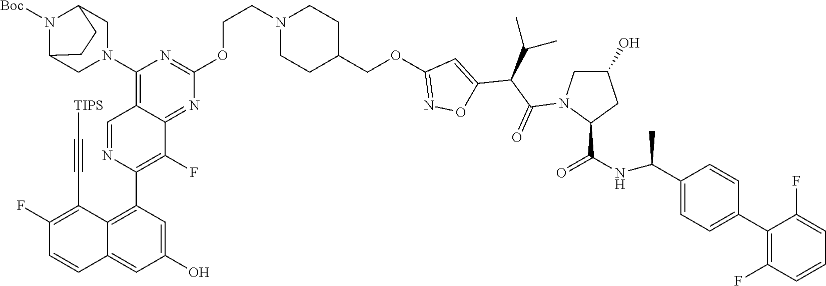

To a solution of (2S,4R)—N—((S)-1-(2′,6′-difluoro-[1,1′-biphenyl]-4-yl)ethyl)-4-hydroxy-1-((R)-3-methyl-2-(3-(piperidin-4-ylmethoxy)isoxazol-5-yl)butanoyl)pyrrolidine-2-carboxamide (109 mg, 0.168 mmol, 1.00 eq, hydrochloride) in dichloromethane (2 mL) and dimethylsulfoxide (2 mL), was added diisopropylethylamine (65 mg, 0.505 mmol, 3.00 eq). The mixture was stirred at 30° C. for 10 min. tert butyl 3-[8-fluoro-7-[7-fluoro-3-hydroxy-8-(2-triisopropylsilylethynyl)-1-naphthyl]-2-(2-oxoethoxy)pyrido [4,3-d]pyrimidin-4-yl]-3,8-diazabicyclo[3.2.1]octane-8-carboxylate (100 mg, 0.13 mmol, 0.78 eq) was added, and the mixture was stirred at 30° C. for 30 min. Then, sodium triacetoxyborohydride (107 mg, 0.505 mmol, 3.00 eq) was added and the mixture was stirred at 30° C. for 2 h. LCMS showed that the desired mass was detected. The mixture was concentrated under reduced pressure to remove the dichloromethane. Ethyl acetate (15 mL) and water (15 mL) were added before the mixture was extracted with ethyl acetate (15 mL×3). The combined organic layers were washed with brine (15 mL×3), dried over anhydrous sodium sulfate, filtered and concentrated under reduced pressure to get the residue. The residue was purified by prep-TLC (9% methanol in dichloromethane) to afford the product (110 mg, 0.08 mmol, 48% yield) as a white solid. LCMS (ESI, m/z): 677.0 [M/2+H]⁺.

Step 7: tert-butyl 3-(2-(2-(4-(((5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy)methyl)piperidin-1-yl)ethoxy)-7-(8-ethynyl-7-fluoro-3-hydroxynaphthalen-1-yl)-8-fluoropyrido[4,3-d]pyrimidin-4-yl)-3,8-diazabicyclo [3.2.1]octane-8-carboxylate

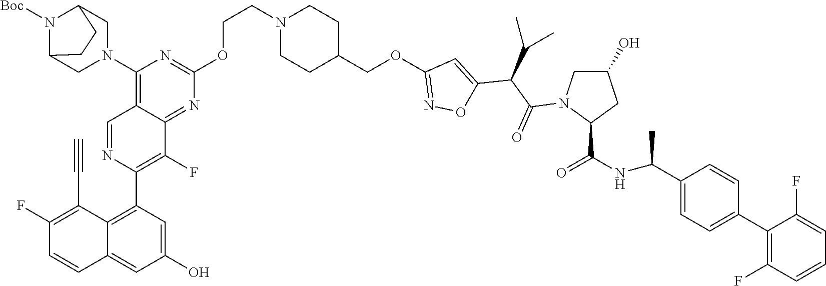 [SMILES: C#Cc1c(F)ccc2cc(O)cc(-c3ncc4c(N5CC6CCC(C5)N6C(=O)OC(C)(C)C)nc(OCCN5CCC(COc6cc([C@H](C(=O)N7C[C@H](O)C[C@H]7C(=O)N[C@@H](C)c7ccc(-c8c(F)cccc8F)cc7)C(C)C)on6)CC5)nc4c3F)c12]

To a solution of tert-butyl 3-(2-(2-(4-(((5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy)methyl)piperidin-1-yl)ethoxy)-8-fluoro-7-(7-fluoro-3-hydroxy-8-((triisopropylsilyl)ethynyl)naphthalen-1-yl)pyrido[4,3-d]pyrimidin-4-yl)-3,8-diazabicyclo[3.2.1]octane-8-carboxylate (110 mg, 0.08 mmol, 1.00 eq) in N,N-dimethylformamide (2 mL), was added cesium fluoride (185 mg, 1.22 mmol, 15.00 eq). The mixture was stirred at 30° C. for 6 h. LCMS showed that the desired mass was detected. Ethyl acetate (10 mL) and water (10 mL) were added before the mixture was extracted with ethyl acetate (10 mL×3). The combined organic layers were washed with brine (10 mL×3), dried over anhydrous sodium sulfate, filtered and concentrated under reduced pressure to afford the crude product (96 mg, 0.08 mmol, 98% yield) as a white solid, which was used in the next step directly. LCMS (ESI, m/z): 598.9 [M/2+H]⁺.

Step 8: (2S,4R)-1-((2R)-2-(3-((1-(2-((4-(3,8-diazabicyclo[3.2.1]octan-3-yl)-7-(8-ethynyl-7-fluoro-3-hydroxynaphthalen-1-yl)-8-fluoropyrido [4,3-d]pyrimidin-2-yl)oxy)ethyl)piperidin-4-yl)methoxy)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide

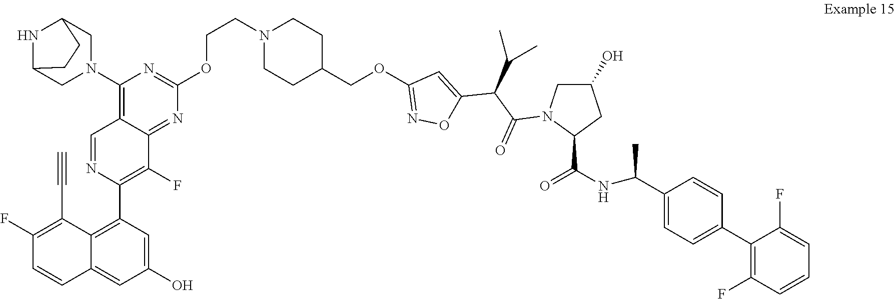 [SMILES: C#Cc1c(F)ccc2cc(O)cc(-c3ncc4c(N5CC6CCC(C5)N6)nc(OCCN5CCC(COc6cc([C@H](C(=O)N7C[C@H](O)C[C@H]7C(=O)N[C@@H](C)c7ccc(-c8c(F)cccc8F)cc7)C(C)C)on6)CC5)nc4c3F)c12]

To a solution of tert-butyl 3-(2-(2-(4-(((5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy)methyl)piperidin-1-yl)ethoxy)-7-(8-ethynyl-7-fluoro-3-hydroxynaphthalen-1-yl)-8-fluoropyrido[4,3-d]pyrimidin-4-yl)-3,8-diazabicyclo[3.2.1]octane-8-carboxylate (96 mg, 0.08 mmol, 1.00 eq) in dichloromethane (2 mL), was added trifluoroacetic acid (1.54 g, 13.51 mmol, 168.30 eq). The mixture was stirred at 20° C. for 0.5 h. LCMS showed that the desired mass was detected. The reaction mixture was concentrated under reduced pressure to get a residue. The residue was purified by preparative high performance liquid chromatography (column: Phenomenex Synergi C18 150*25 mm*10 um; mobile phase: [water(FA)-ACN]; B %: 18%-38%, 10 min) to afford the product (25.7 mg, 0.02 mmol, 28% yield, 100% purity, formate[1]) as a yellow solid. LCMS (ESI) m/z: 1096.4 [M+H]⁺, ¹HNMR (400 MHz, DMSO-d₆) δ 10.32-10.05 (m, 1H), 9.06 (s, 1H), 8.31 (d, J=7.6 Hz, 1H), 8.16 (s, 1H), 7.98 (dd, J=6.0, 9.2 Hz, 1H), 7.51-7.43 (m, 3H), 7.40 (d, J=2.4 Hz, 1H), 7.43-7.31 (m, 4H), 7.25-7.17 (m, 3H), 6.08 (s, 1H), 4.94-4.90 (m, 1H), 4.56-4.36 (m, 5H), 4.28 (s, 1H), 4.03-3.94 (m, 3H), 3.73 (d, J=9.2 Hz, 3H), 3.68 (s, 2H), 3.00-2.91 (m, 2H), 2.76-2.69 (m, 2H), 2.27-2.23 (m, 1H), 2.11-1.94 (m, 4H), 1.84-1.71 (m, 6H), 1.70-1.59 (m, 3H), 1.48 (d, J=6.8 Hz, 1H), 1.37 (d, J=7.2 Hz, 3H), 1.32-1.14 (m, 3H), 0.97 (d, J=6.8 Hz, 3H), 0.83 (d, J=6.8 Hz, 3H).

Example 16: (2S,4R)-1-[(2R)-2-[3-[7-[2-[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-2,7-diazaspiro[3.5]nonan-2-yl]isoxazol-5-yl]-3-methyl-butanoyl]-N-[(1S)-1-[4-(2,6-difluorophenyl)phenyl]ethyl]-4-hydroxy-pyrrolidine-2-carboxamide

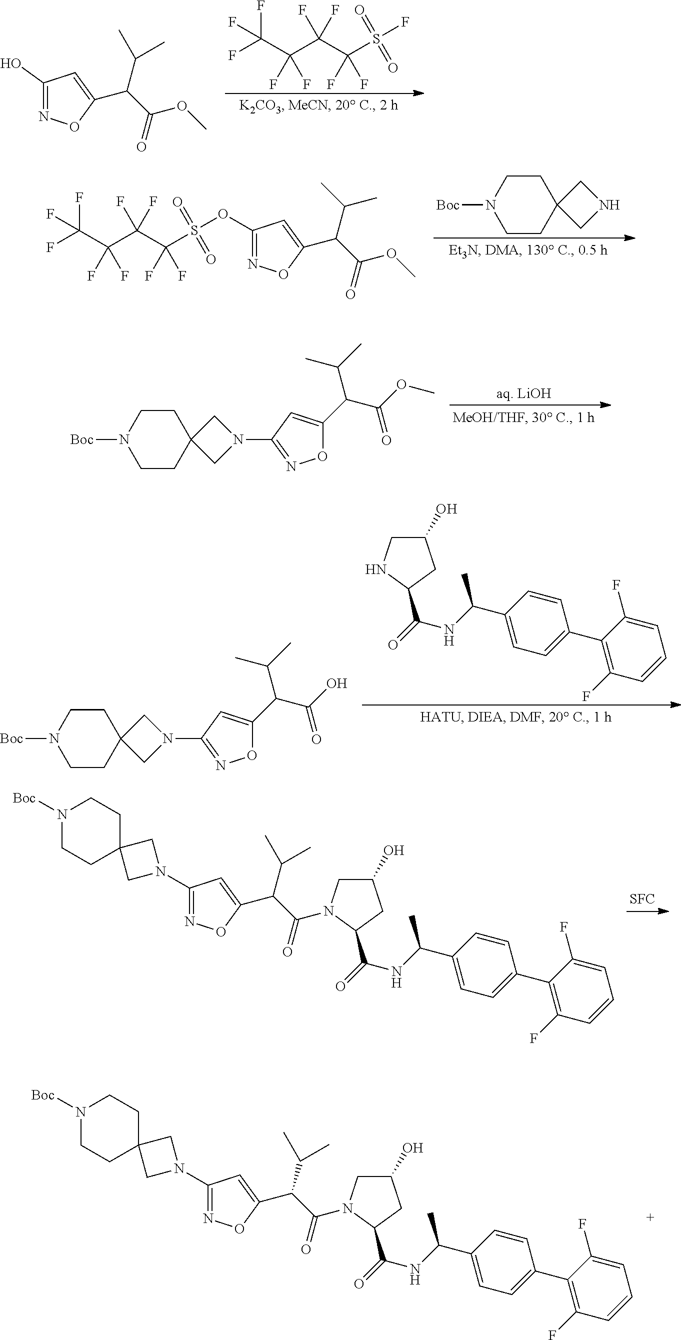

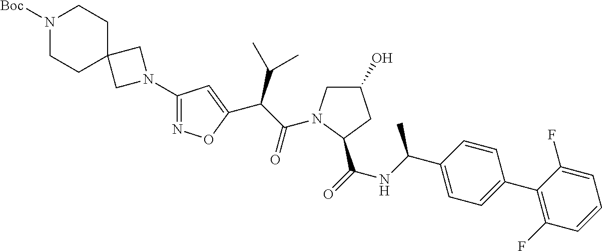 [SMILES: CC(C)[C@@H](C(=O)N1C[C@H](O)C[C@H]1C(=O)N[C@@H](C)c1ccc(-c2c(F)cccc2F)cc1)c1cc(N2CC3(CCN(C(=O)OC(C)(C)C)CC3)C2)no1]

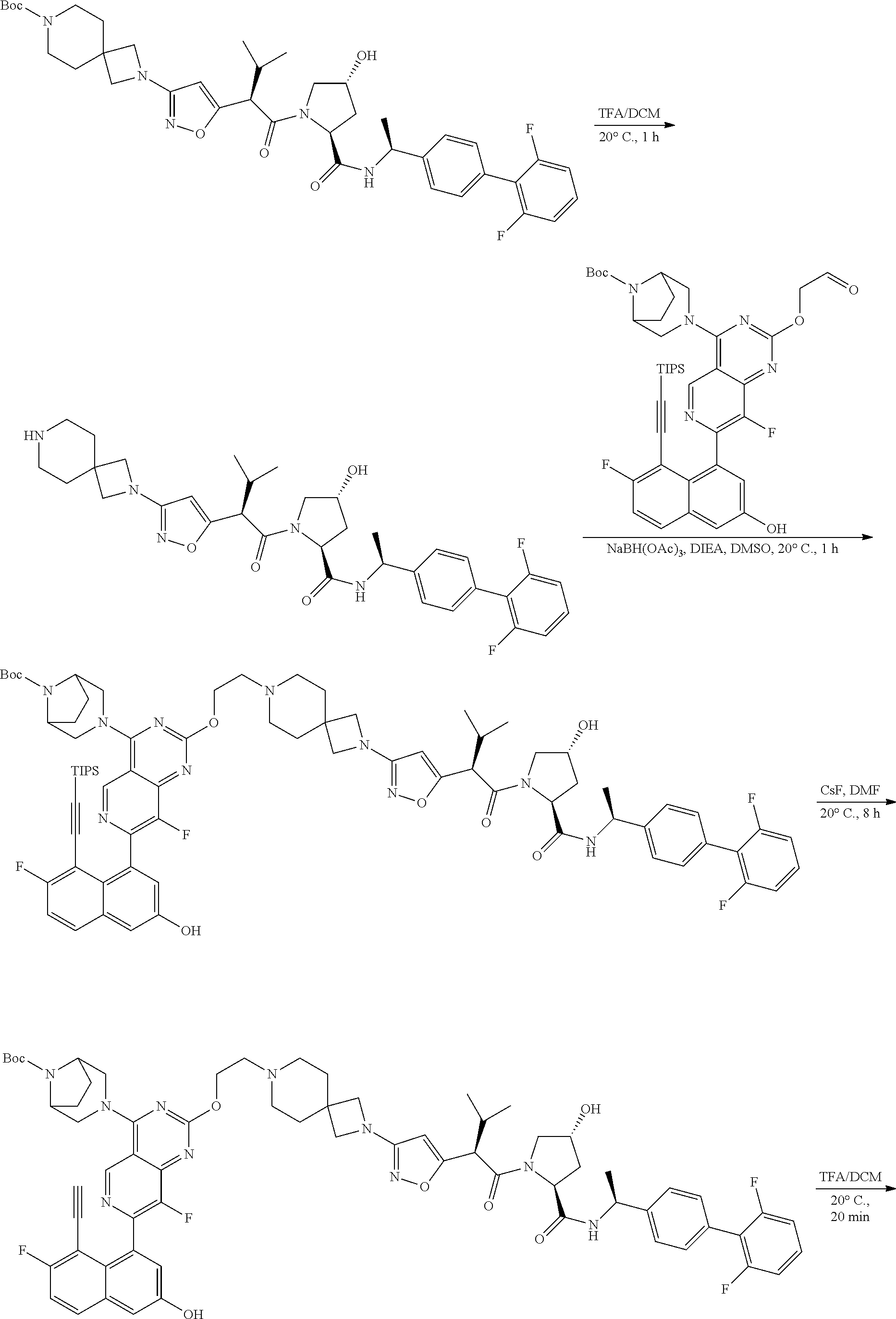

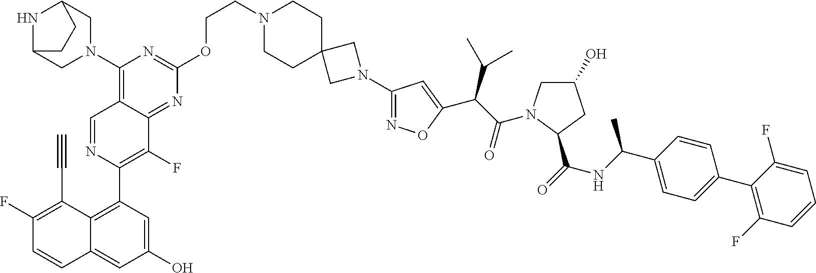 [SMILES: C#Cc1c(F)ccc2cc(O)cc(-c3ncc4c(N5CC6CCC(C5)N6)nc(OCCN5CCC6(CC5)CN(c5cc([C@H](C(=O)N7C[C@H](O)C[C@H]7C(=O)N[C@@H](C)c7ccc(-c8c(F)cccc8F)cc7)C(C)C)on5)C6)nc4c3F)c12]

Step 1: methyl 3-methyl-2-[3-(1,1,2,2,3,3,4,4,4-nonafluorobutylsulfonyloxy)isoxazol-5-yl]butanoate

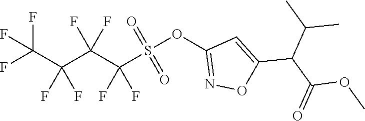 [SMILES: COC(=O)C(c1cc(OS(=O)(=O)C(F)(F)C(F)(F)C(F)(F)C(F)(F)F)no1)C(C)C]

To a solution of methyl 2-(3-hydroxyisoxazol-5-yl)-3-methyl-butanoate (2 g, 10.04 mmol) in acetonitrile (20 mL) was added 1,1,2,2,3,3,4,4,4-nonafluorobutane-1-sulfonyl fluoride (4.55 g, 15.06 mmol) and potassium carbonate (4.16 g, 30.12 mmol). The mixture was stirred at 20° C. for 2 h. The reaction mixture was diluted with water (300 mL) and extracted with ethyl acetate (300 mL×3). The combined organic layers were washed with brine (100 mL×2), dried over sodium sulfate, filtered and concentrated under reduced pressure to give a residue. The residue was purified by column chromatography on silica gel (petroleum ether: ethyl acetate=20:1 to 3:1) to afford the desired product (3.9 g, 8.10 mmol, 80% yield) as a colorless liquid. ¹H NMR: (400 MHz, CHLOROFORM-d) δ: 6.42 (s, 1H), 3.77 (s, 3H), 3.67 (d, J=8.4 Hz, 1H), 2.45-2.36 (m, 1H), 1.03 (d, J=6.8 Hz, 3H), 0.94 (d, J=6.8 Hz, 3H).

Step 2: tert-butyl 2-[5-(1-methoxycarbonyl-2-methyl-propyl)isoxazol-3-yl]-2,7-diazaspiro[3.5]nonane-7-carboxylate

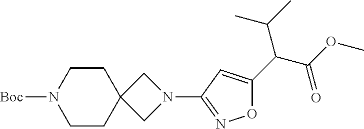 [SMILES: COC(=O)C(c1cc(N2CC3(CCN(C(=O)OC(C)(C)C)CC3)C2)no1)C(C)C]

To a solution of tert-butyl 2,7-diazaspiro[3.5]nonane-7-carboxylate (3.67 g, 16.21 mmol) in N,N-dimethylacetamide (20 mL) was added triethylamine (2.46 g, 24.31 mmol) at 130° C. After addition, then methyl 3-methyl-2-[3-(1,1,2,2,3,3,4,4,4-nonafluorobutylsulfonyloxy)isoxazol-5-yl]butanoate (3.9 g, 8.10 mmol) in N—N-dimethylacetamide (20 mL) was added dropwise at 130° C. The resulting mixture was stirred at 130° C. for 0.5 h. The reaction mixture was quenched by the addition of hydrochloric acid (1M, 30 mL) at 25° C. The mixture was diluted with water (150 mL) and extracted with ethyl acetate (100 mL×3). The combined organic layers were washed with brine (150 mL×3), dried over anhydrous sodium sulfate, filtered and concentrated under reduced pressure to give a residue. The crude product was purified by preparative high performance liquid chromatography (column: Phenomenex luna C18 (250*70 mm, 10 um); mobile phase: [water(TFA)-ACN]; B %: 50%-80%, 21 min) to give the product (1.65 g, 4.05 mmol, 50% yield) as a brown oil. LCMS (ESI, m/z): 408.1 [M+H]⁺.

Step 3: 2-[3-(7-tert-butoxycarbonyl-2,7-diazaspiro[3.5]nonan-2-yl)isoxazol-5-yl]-3-methyl-butanoic acid

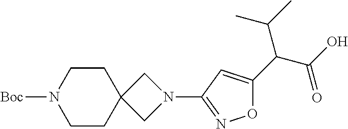 [SMILES: CC(C)C(C(=O)O)c1cc(N2CC3(CCN(C(=O)OC(C)(C)C)CC3)C2)no1]

To a solution of tert-butyl 2-[5-(1-methoxycarbonyl-2-methyl-propyl)isoxazol-3-yl]-2,7-diazaspiro[3.5]nonane-7-carboxylate (1.65 g, 4.05 mmol) in tetrahydrofuran (20 mL) and methanol (20 mL) was added lithium hydroxide (2 M, 20 mL). The mixture was stirred at 30° C. for 1 h. The reaction mixture was quenched by the addition of aqueous hydrochloride (1M, 30 mL) at 25° C., and the mixture was diluted with water (150 mL) and extracted with ethyl acetate (100 mL×3). The combined organic layers were washed with brine (150 mL×3), dried over sodium sulfate, filtered and concentrated under reduced pressure to give a residue. Compound 2-[3-(7-tert-butoxycarbonyl-2,7-diazaspiro[3.5]nonan-2-yl)isoxazol-5-yl]-3-methyl-butanoic acid (1.21 g, 3.08 mmol, 76% yield) was obtained as a white solid. LCMS (ESI, m/z): 394.1 [M+H]⁺.

Step 4: tert-butyl 2-[5-[1-[(2S,4R)-2-[[(1S)-1-[4-(2,6-difluorophenyl)phenyl]ethyl]carbamoyl]-4-hydroxy-pyrrolidine-1-carbonyl]-2-methyl-propyl]isoxazol-3-yl]-2,7-diazaspiro[3.5]nonane-7-carboxylate

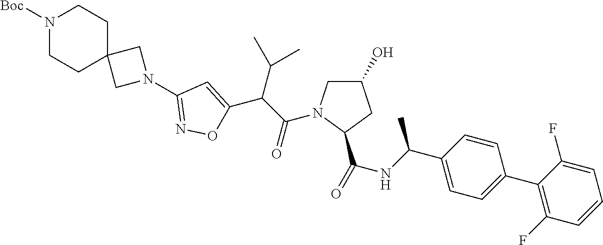 [SMILES: CC(C)C(C(=O)N1C[C@H](O)C[C@H]1C(=O)N[C@@H](C)c1ccc(-c2c(F)cccc2F)cc1)c1cc(N2CC3(CCN(C(=O)OC(C)(C)C)CC3)C2)no1]

To a solution of 2-[3-(7-tert-butoxycarbonyl-2,7-diazaspiro[3.5]nonan-2-yl)isoxazol-5-yl]-3-methyl-butanoic acid (1 g, 2.54 mmol) and (2S,4R)—N-[(1S)-1-[4-(2,6-difluorophenyl)phenyl]ethyl]-4-hydroxy-pyrrolidine-2-carboxamide (1.17 g, 2.54 mmol, trifluoroacetate) in N,N-dimethylformamide (50 mL) was added O-(7-Azabenzotriazol-1-yl)-N,N,N′,N′-tetramethyluronium exafluorophosphate (1.16 g, 3.05 mmol) and N,N-diisopropylethylamine (1.64 g, 12.71 mmol). The mixture was stirred at 20° C. for 1 h. After the reaction was completed, the mixture was diluted with water (200 mL) and extracted with ethyl acetate (200 mL×3). The combined organic layers were washed with brine (100 mL×2), dried over sodium sulfate, filtered and concentrated under reduced pressure to give a residue. The residue was purified by preparative high performance liquid chromatography (column: Phenomenex luna C18 150*40 mm*15 um; mobile phase: [water(FA)-ACN]; B %: 58%-78%, 10 min) to afford the desired product (890 mg, 1.23 mmol, 48% yield) as a white solid. LCMS (ESI, m/z): 722.4 [M+H]⁺.

Step 5: tert-butyl 2-[5-[(1R)-1-[(2S,4R)-2-[[(1S)-1-[4-(2,6-difluorophenyl)phenyl]ethyl]carbamoyl]-4-hydroxy-pyrrolidine-1-carbonyl]-2-methyl-propyl]isoxazol-3-yl]-2,7-diazaspiro[3.5]nonane-7-carboxylate

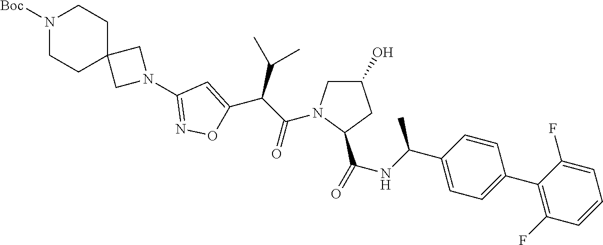 [SMILES: CC(C)[C@@H](C(=O)N1C[C@H](O)C[C@H]1C(=O)N[C@@H](C)c1ccc(-c2c(F)cccc2F)cc1)c1cc(N2CC3(CCN(C(=O)OC(C)(C)C)CC3)C2)no1]

The mixture of diastereoisomers tert-butyl 2-[5-[1-[(2S,4R)-2-[[(1R)-1-[4-(2,6-difluorophenyl)phenyl]ethyl] carbamoyl]-4-hydroxy-pyrrolidine-1-carbonyl]-2-methyl-propyl]isoxazol-3-yl]-2,7-diazaspiro [3.5]nonane-7-carboxylate and tert-butyl 2-[5-[1-[(2S,4R)-2-[[(1S)-1-[4-(2,6-difluorophenyl)phenyl]ethyl] carbamoyl]-4-hydroxy-pyrrolidine-1-carbonyl]-2-methyl-propyl]isoxazol-3-yl]-2,7-diazaspiro [3.5]nonane-7-carboxylate (500 mg, 0.69 mmol) was separated by SFC (column:DAICEL CHIRALCEL OD(250 mm*30 mm, 10 um); mobile phase:[0.1% NH3H2O IPA]; B %: 35%-35%, 3.9 mn), and the second eluent was identified as the desired diastereoisomer tert-butyl 2-[5-[(1R)-1-[(2S,4R)-2-[[(1S)-1-[4-(2,6-difluorophenyl)phenyl]ethyl]carbamoyl]-4-hydroxy-pyrrolidine-1-carbonyl]-2-methyl-propyl]isoxazol-3-yl]-2,7-diazaspiro[3.5]nonane-7-carboxylate. The product (200 mg, 0.26 mmol, 38% yield, 97% purity) was obtained as a colorless oil. LCMS (ESI, m/z): 722.4 [M+H]⁺.

Step 6: (2S,4R)-1-[(2R)-2-[3-(2,7-diazaspiro[3.5]nonan-2-yl)isoxazol-5-yl]-3-methyl-butanoyl]-N-[(1S)-1-[4-(2,6-difluorophenyl)phenyl]ethyl]-4-hydroxy-pyrrolidine-2-carboxamide

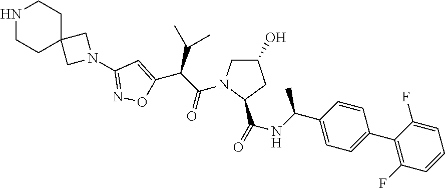 [SMILES: CC(C)[C@@H](C(=O)N1C[C@H](O)C[C@H]1C(=O)N[C@@H](C)c1ccc(-c2c(F)cccc2F)cc1)c1cc(N2CC3(CCNCC3)C2)no1]

To a solution of tert-butyl 2-[5-[(1R)-1-[(2S,4R)-2-[[(1S)-1-[4-(2,6-difluorophenyl)phenyl]ethyl] carbamoyl]-4-hydroxy-pyrrolidine-1-carbonyl]-2-methyl-propyl]isoxazol-3-yl]-2,7-diazaspiro [3.5]nonane-7-carboxylate (150 mg, 0.20 mmol) in dichloromethane (2 mL) was added trifluoroacetic acid (3.08 g, 27.01 mmol). The mixture was stirred at 20° C. for 10 min. The reaction mixture was filtered and concentrated under reduced pressure to give the desired product (120 mg, 0.19 mmol, 92% yield) as a red solid. LCMS (ESI, m/z): 622.2 [M+H]⁺

Step 7: tert-butyl 3-[2-[2-[2-[5-[(1R)-1-[(2S,4R)-2-[[(1S)-1-[4-(2,6-difluorophenyl)phenyl]ethyl] carbamoyl]-4-hydroxy-pyrrolidine-1-carbonyl]-2-methyl-propyl]isoxazol-3-yl]-2,7-diazaspiro [3.5]nonan-7-yl]ethoxy]-8-fluoro-7-[7-fluoro-3-hydroxy-8-(2-triisopropylsilylethynyl)-1-naphthyl]pyrido[4,3-d]pyrimidin-4-yl]-3,8-diazabicyclo[3.2.1]octane-8-carboxylate

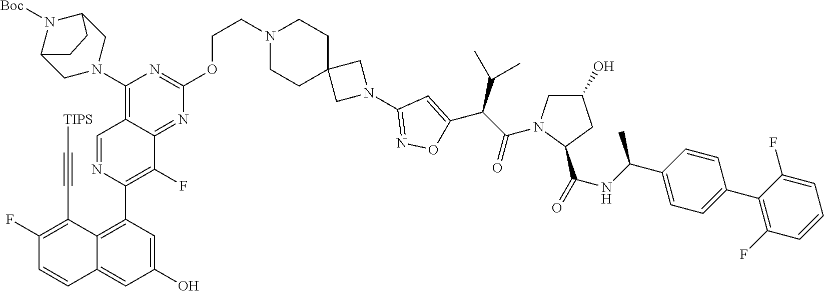

To a solution of (2S,4R)-1-[(2R)-2-[3-(2,7-diazaspiro[3.5]nonan-2-yl)isoxazol-5-yl]-3-methyl-butanoyl]-N-[(1S)-1-[4-(2,6-difluorophenyl)phenyl]ethyl]-4-hydroxy-pyrrolidine-2-carboxamide (90 mg, 0.15 mmol) and tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (100 mg, 0.13 mmol) in dimethyl sulfoxide (2 mL) was added sodium triacetoxyborohydride (83 mg, 0.40 mmol) and N,N-diisopropylethylamine (68 mg, 0.53 mmol). The mixture was stirred at 20° C. for 0.5 h. The reaction mixture was diluted with water (50 mL) and extracted with ethyl acetate (50 mL×3). The combined organic layers were washed with brine (50 mL×2), dried over sodium sulfate, filtered and concentrated under reduced pressure to give a residue. The residue was purified by prep-TLC (9% methanol in dichloromethane) to afford the desired product (110 mg, 0.08 mmol, 61% yield) as a white solid. LCMS (ESI, m/z): 1364.3 [M+H]⁺.

Step 8: tert-butyl 3-[2-[2-[2-[5-[(1R)-1-[(2S,4R)-2-[[(1S)-1-[4-(2,6-difluorophenyl)phenyl]ethyl] carbamoyl]-4-hydroxy-pyrrolidine-1-carbonyl]-2-methyl-propyl]isoxazol-3-yl]-2,7-diazaspiro [3.5]nonan-7-yl]ethoxy]-7-(8-ethynyl-7-fluoro-3-hydroxy-1-naphthyl)-8-fluoro-pyrido[4,3-d]pyrimidin-4-yl]-3,8-diazabicyclo[3.2.1]octane-8-carboxylate

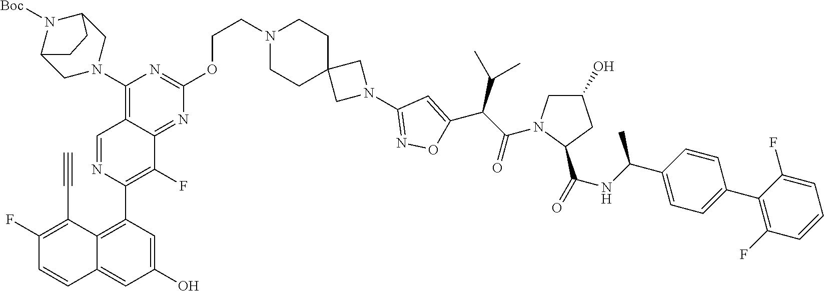 [SMILES: C#Cc1c(F)ccc2cc(O)cc(-c3ncc4c(N5CC6CCC(C5)N6C(=O)OC(C)(C)C)nc(OCCN5CCC6(CC5)CN(c5cc([C@H](C(=O)N7C[C@H](O)C[C@H]7C(=O)N[C@@H](C)c7ccc(-c8c(F)cccc8F)cc7)C(C)C)on5)C6)nc4c3F)c12]

To a solution of tert-butyl 3-[2-[2-[2-[5-[(1R)-1-[(2S,4R)-2-[[(1S)-1-[4-(2,6-difluorophenyl) phenyl]ethyl]carbamoyl]-4-hydroxy-pyrrolidine-1-carbonyl]-2-methyl-propyl]isoxazol-3-yl]-2,7-diazaspiro[3.5]nonan-7-yl]ethoxy]-8-fluoro-7-[7-fluoro-3-hydroxy-8-(2-triisopropylsilylethynyl)-1-naphthyl]pyrido[4,3-d]pyrimidin-4-yl]-3,8-diazabicyclo[3.2.1]octane-8-carboxylate (110 mg, 0.08 mmol) in N,N-dimethylformamide (2 mL) was added cesium fluoride (183 mg, 1.21 mmol). The mixture was stirred at 20° C. for 8 h. The reaction mixture was filtered and the filtrate was concentrated under reduced pressure to give the crude product (90 mg, 0.07 mmol, 92% yield) as a white solid. LCMS (ESI, m/z): 1207.4 [M+H]⁺.

Step 9: (2S,4R)-1-[(2R)-2-[3-[7-[2-[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-2,7-diazaspiro[3.5]nonan-2-yl]isoxazol-5-yl]-3-methyl-butanoyl]-N-[(1S)-1-[4-(2,6-difluorophenyl)phenyl]ethyl]-4-hydroxy-pyrrolidine-2-carboxamide

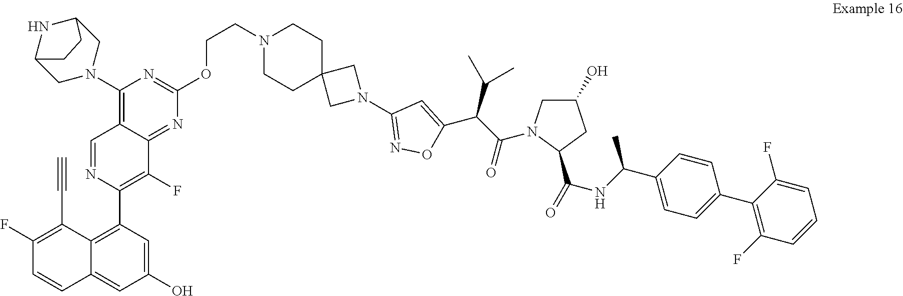 [SMILES: C#Cc1c(F)ccc2cc(O)cc(-c3ncc4c(N5CC6CCC(C5)N6)nc(OCCN5CCC6(CC5)CN(c5cc([C@H](C(=O)N7C[C@H](O)C[C@H]7C(=O)N[C@@H](C)c7ccc(-c8c(F)cccc8F)cc7)C(C)C)on5)C6)nc4c3F)c12]

To a solution of tert-butyl 3-[2-[2-[2-[5-[(1R)-1-[(2S,4R)-2-[[(1S)-1-[4-(2,6-difluorophenyl) phenyl]ethyl]carbamoyl]-4-hydroxy-pyrrolidine-1-carbonyl]-2-methyl-propyl]isoxazol-3-yl]-2,7-diazaspiro[3.5]nonan-7-yl]ethoxy]-7-(8-ethynyl-7-fluoro-3-hydroxy-1-naphthyl)-8-fluoro-pyrido [4,3-d]pyrimidin-4-yl]-3,8-diazabicyclo[3.2.1]octane-8-carboxylate (90 mg, 0.07 mmol) in dichloromethane (2 mL) was added trifluoroacetic acid (3.08 g, 27.01 mmol). The mixture was stirred at 20° C. for 20 min. The reaction mixture was diluted with water (50 mL) and extracted with ethyl acetate (50 mL×3). The combined organic layers were washed with brine (50 mL×2), dried over anhydrous sodium sulfate, filtered and concentrated under reduced pressure to give a residue. The residue was purified by preparative high performance liquid chromatography (column: Phenomenex Synergi C18 150*25 mm*10 um; mobile phase: [water(FA)-ACN]; B %: 12%-42%, 10 min) to afford the desired product (16 mg, 0.01 mmol, 19% yield, 98% purity) as a white solid. LCMS (ESI, m/z): 1107.3 [M+H]⁺. ¹H NMR: (400 MHz, DMSO) δ: 10.19-10.12 (m, 1H), 9.08-9.03 (m, 1H), 8.44-8.38 (m, 1H), 8.17-8.13 (m, 1H), 8.01-7.95 (m, 1H), 7.44 (s, 1H), 7.39 (s, 6H), 7.25-7.16 (m, 4H), 5.83 (s, 1H), 4.97-4.89 (m, 1H), 4.54-4.48 (m, 1H), 4.47-4.43 (m, 2H), 4.43-4.41 (m, 1H), 4.39-4.32 (m, 2H), 4.30-4.25 (m, 1H), 3.96-3.92 (m, 1H), 3.72 (br s, 1H), 3.70-3.67 (m, 2H), 3.67-3.62 (m, 2H), 3.62-3.59 (m, 1H), 3.43-3.39 (m, 1H), 3.38 (br s, 1H), 2.27-2.13 (m, 2H), 2.06 (br d, J=2.0 Hz, 1H), 1.78-1.65 (m, 10H), 1.39 (d, J=7.2 Hz, 3H), 0.94 (br d, J=6.4 Hz, 3H), 0.73 (s, 4H).

Example 17: (2S,4R)-1-((2R)-2-(3-((1-(2-((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)ethyl)piperidin-4-yl)methoxy)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide

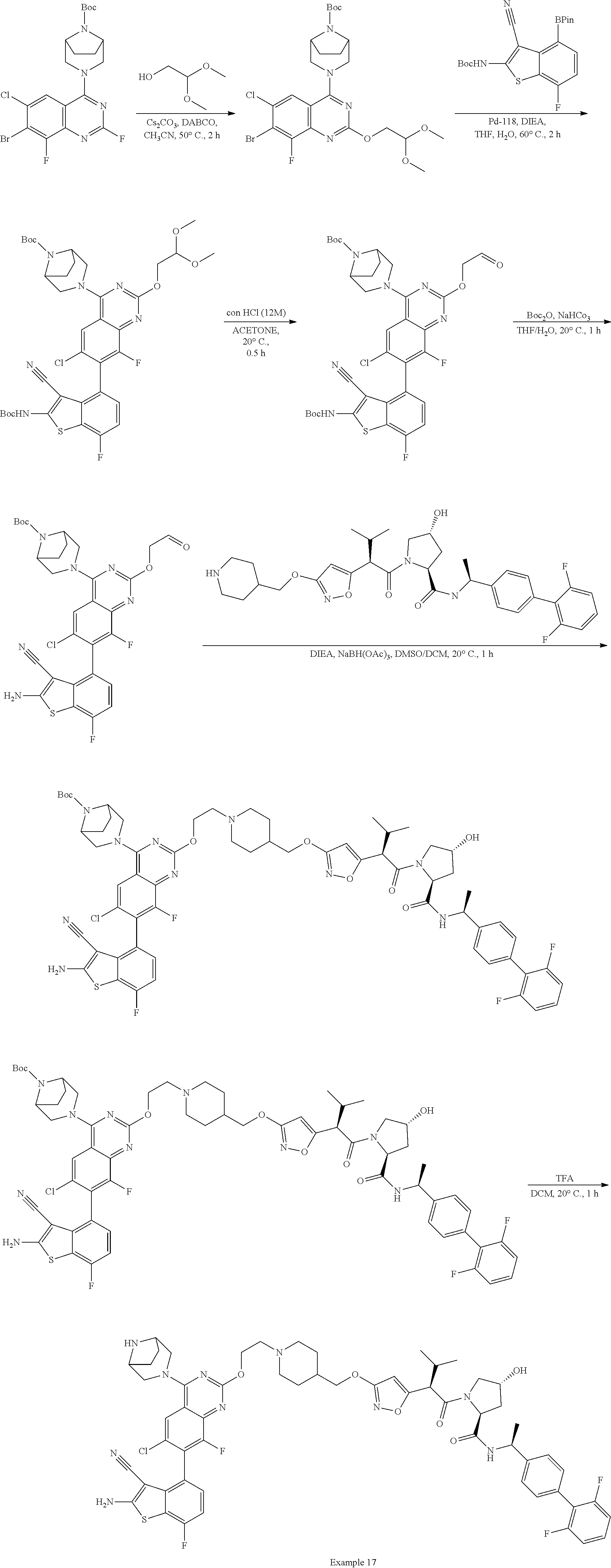

Step 1: tert-butyl (1R,5S)-3-(7-bromo-6-chloro-2-(2,2-dimethoxyethoxy)-8-fluoroquinazolin-4-yl)-3,8-diazabicyclo [3.2.1]octane-8-carboxylate

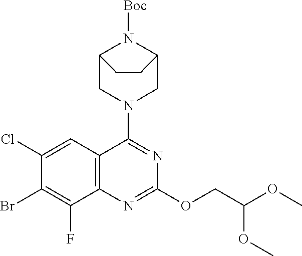 [SMILES: COC(COc1nc(N2CC3CCC(C2)N3C(=O)OC(C)(C)C)c2cc(Cl)c(Br)c(F)c2n1)OC]

To a solution of tert-butyl (1R,5S)-3-(7-bromo-6-chloro-2,8-difluoroquinazolin-4-yl)-3,8-diazabicyclo[3.2.1]octane-8-carboxylate (2 g, 4 mmol, 1 eq) and 2,2-dimethoxyethan-1-ol (433 mg, 4.00 mmol, 1 eq) in acetonitrile (10 mL) was added cesium carbonate (2.7 g, 8.00 mmol, 2 eq) and 1,4-diazabicyclo[2.2.2]octane (46 mg, 0.4 mmol, 0.1 eq). The mixture was stirred at 50° C. for 2 h. LCMS showed the starting material was consumed completely and one main peak with desired mass was detected. The reaction mixture was diluted with water (20 mL) and extracted with ethyl acetate (20 mL×3). The combined organic layers were washed with brine (20 mL×3), dried over anhydrous sodium sulfate, and filtered. The filtrate was concentrated under reduced pressure to give a residue. The residue was purified by column chromatography (petroleum ether/ethyl acetate=3/1) to afford the desired product (1.9 g, 3 mmol, 80% yield) as a white solid. ¹H NMR (400 MHz, CDCl₃) δ 7.70 (d, J=2.0 Hz, 1H), 4.83 (t, J=5.2 Hz, 1H), 4.48 (d, J=5.6 Hz, 2H), 4.32 (d, J=12.4 Hz, 4H), 3.70-3.52 (m, 2H), 3.47 (s, 6H), 2.00-1.90 (m, 2H), 1.74-1.73 (m, 1H), 1.83-1.71 (m, 2H), 1.52 (s, 9H).

Step 2: tert-butyl 3-(7-(2-((tert-butoxycarbonyl)amino)-3-cyano-7-fluorobenzo[b]thiophen-4-yl)-6-chloro-2-(2,2-dimethoxyethoxy)-8-fluoroquinazolin-4-yl)-3,8-diazabicyclo[3.2.1]octane-8-carboxylate

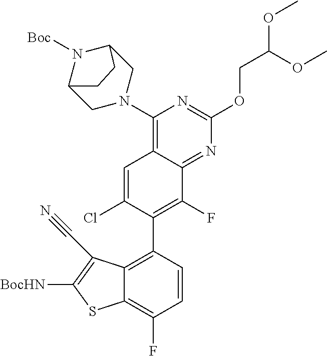 [SMILES: COC(COc1nc(N2CC3CCC(C2)N3C(=O)OC(C)(C)C)c2cc(Cl)c(-c3ccc(F)c4sc(NC(=O)OC(C)(C)C)c(C#N)c34)c(F)c2n1)OC]

A mixture of tert-butyl (1R,5S)-3-(7-bromo-6-chloro-2-(2,2-dimethoxyethoxy)-8-fluoroquinazolin-4-yl)-3,8-diazabicyclo[3.2.1]octane-8-carboxylate (870 mg, 1.5 mmol, 1 eq), tert-butyl (3-cyano-7-fluoro-4-(4,4,5,5-tetramethyl-1,3,2-dioxaborolan-2-yl)benzo[b]thiophen-2-yl)carbamate (0.95 g, 2.3 mmol, 1.5 eq), ditert-butyl(cyclopentyl)phosphane; dichloropalladium; iron (98 mg, 0.15 mmol, 0.1 eq), N,N-diisopropylethylamine (0.6 g, 4.5 mmol, 3 eq) in tetrahydrofuran (4 mL) and water (1 mL) was degassed and purged with nitrogen for 3 times, and then the mixture was stirred at 60° C. for 2 h under nitrogen atmosphere. LCMS showed the starting material as consumed completely and one main peak with desired mass was detected. The reaction mixture was diluted with water (20 mL) and extracted with ethyl acetate (20 mL×3). The combined organic layers were washed with brine (20 mL×3), dried over anhydrous sodium sulfate, and filtered. The filtrate was concentrated under reduced pressure to give a residue. The residue was purified by prep-HPLC (column: Phenomenex luna C18 150*40 mm*15 um; mobile phase: [water (formic acid)-acetonitrile]; B %: 80%-100%, 10 min) to afford the product (242 mg, 0.3 mmol, 20% yield) as a yellow solid. ¹H NMR (400 MHz, CDCl₃) δ 7.75 (s, 2H), 7.31 (dd, J=4.8, 8.4 Hz, 1H), 7.16 (t, J=8.8 Hz, 1H), 5.31 (s, 1H), 4.84 (t, J=5.2 Hz, 1H), 4.54-4.26 (m, 6H), 3.69-3.53 (m, 2H), 3.47 (s, 5H), 1.99-1.90 (m, 2H), 1.89-1.77 (m, 2H), 1.57 (s, 9H), 1.53 (s, 9H).

Step 3: 4-(4-(3,8-diazabicyclo[3.2.1]octan-3-yl)-6-chloro-8-fluoro-2-(2-oxoethoxy)quinazolin-7-yl)-2-amino-7-fluorobenzo[b]thiophene-3-carbonitrile

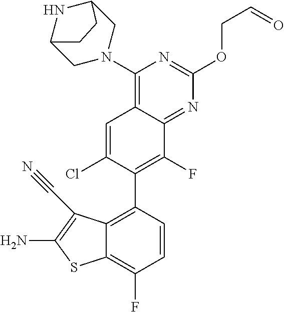 [SMILES: N#Cc1c(N)sc2c(F)ccc(-c3c(Cl)cc4c(N5CC6CCC(C5)N6)nc(OCC=O)nc4c3F)c12]

To a solution of tert-butyl 3-(7-(2-((tert-butoxycarbonyl)amino)-3-cyano-7-fluorobenzo[b]thiophen-4-yl)-6-chloro-2-(2,2-dimethoxyethoxy)-8-fluoroquinazolin-4-yl)-3,8-diazabicyclo[3.2.1]octane-8-carboxylate (100 mg, 0.13 mmol, 1 eq) in acetone (2 mL) was added hydrochloric acid (12 M, 0.2 mL, 20 eq). The mixture was stirred at 20° C. for 30 min. LCMS showed the starting material was consumed completely and one main peak with desired mass was detected. The reaction mixture was concentrated under reduced pressure to remove solvent. The product 2-amino-4-[6-chloro-4-(3,8-diazabicyclo[3.2.1]octan-3-yl)-8-fluoro-2-(2-oxoethoxy)quinazolin-7-yl]-7-fluoro-benzothiophene-3-carbonitrile (80 mg, crude) was obtained as a yellow solid, which was used into the next step without further purification. LCMS (ESI, m/z): 559.0 [M+18]⁺.

Step 4: tert-butyl 3-(7-(2-amino-3-cyano-7-fluorobenzo[b]thiophen-4-yl)-6-chloro-8-fluoro-2-(2-oxoethoxy)quinazolin-4-yl)-3,8-diazabicyclo[3.2.1]octane-8-carboxylate

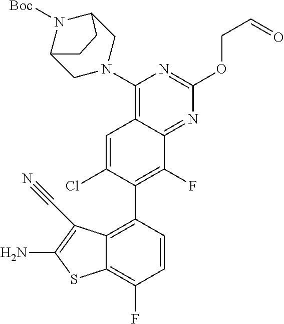 [SMILES: CC(C)(C)OC(=O)N1C2CCC1CN(c1nc(OCC=O)nc3c(F)c(-c4ccc(F)c5sc(N)c(C#N)c45)c(Cl)cc13)C2]

To a solution of 4-(4-(3,8-diazabicyclo[3.2.1]octan-3-yl)-6-chloro-8-fluoro-2-(2-oxoethoxy)quinazolin-7-yl)-2-amino-7-fluorobenzo[b]thiophene-3-carbonitrile (97 mg, 0.18 mmol, 1 eq) in tetrahydrofuran (1 mL) and water (1 mL) was added di-tert-butyl dicarbonate (86 mg, 0.39 mmol, 2.2 eq) and sodium bicarbonate (512 mg, 6.10 mmol, 34 eq). The mixture was stirred at 20° C. for 1 h. LCMS showed the starting material was consumed completely and two peak with desired mass was detected. The reaction mixture was diluted with water (10 mL) and extracted with ethyl acetate (10 mL×3). The combined organic layers were washed with brine (10 mL×3), dried over anhydrous sodium sulfate, filtered. The filtrate was concentrated under reduced pressure to give a residue. The residue was purified by prep-TLC (dichloromethane/methanol=10/1) to afford the product (223 mg, crude) as a yellow solid. LCMS (ESI, m/z): 640.9 [M]⁺.

Step 5: tert-butyl 3-(7-(2-amino-3-cyano-7-fluorobenzo[b]thiophen-4-yl)-6-chloro-2-(2-(4-(((5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy)methyl)piperidin-1-yl)ethoxy)-8-fluoroquinazolin-4-yl)-3,8-diazabicyclo[3.2.1]octane-8-carboxylate

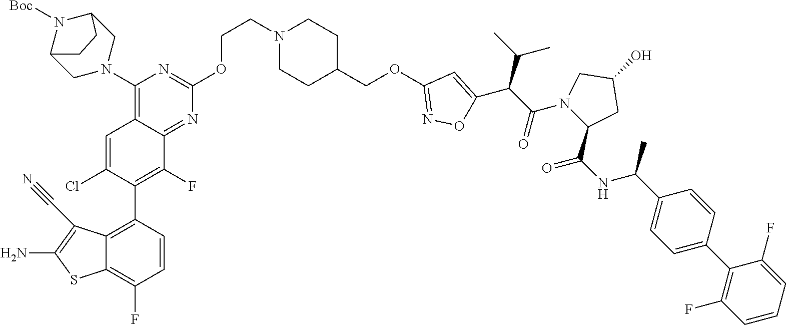 [SMILES: CC(C)[C@@H](C(=O)N1C[C@H](O)C[C@H]1C(=O)N[C@@H](C)c1ccc(-c2c(F)cccc2F)cc1)c1cc(OCC2CCN(CCOc3nc(N4CC5CCC(C4)N5C(=O)OC(C)(C)C)c4cc(Cl)c(-c5ccc(F)c6sc(N)c(C#N)c56)c(F)c4n3)CC2)no1]

To a solution of tert-butyl 3-(7-(2-amino-3-cyano-7-fluorobenzo[b]thiophen-4-yl)-6-chloro-8-fluoro-2-(2-oxoethoxy)quinazolin-4-yl)-3,8-diazabicyclo[3.2.1]octane-8-carboxylate (73 mg, 0.11 mmol, 1 eq) and (2S,4R)—N-[(1S)-1-[4-(2,6-difluorophenyl)phenyl]ethyl]-4-hydroxy-1-[(2R)-3-methyl-2-[3-(4-piperidylmethoxy)isoxazol-5-yl]butanoyl]pyrrolidine-2-carboxamide (70 mg, 0.11 mmol, 1 eq) in dimethylsulfoxide (1 mL) and dichloromethane (1 mL) was added N,N-diisopropylethylamine (44 mg, 0.3 mmol, 3 eq), the mixture was stirred at 20° C. for 30 mn. Then sodium triacetoxyborohydride (72 mg, 0.3 mmol, 3 eq) was added, and the mixture was stirred at 20° C. for 30 min. LCMS showed the starting material was consumed completely and one main peak with desired mass was detected. The reaction mixture was diluted with water (10 mL) and extracted with ethyl acetate (10 mL×3). The combined organic layers were washed with brine (10 mL×3), dried over anhydrous sodium sulfate, filtered. The filtrate was concentrated under reduced pressure to give a residue. The residue was purified by prep-TLC (dichloromethane/methanol=10/1) to afford the desired product (68 mg, 0.04 mmol, 36% yield) as a white solid. LCMS (ESI, m/z): 1235.1 [M]⁺.

Step 6: (2S,4R)-1-((2R)-2-(3-((1-(2-((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)ethyl)piperidin-4-yl)methoxy)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide (Example 17)

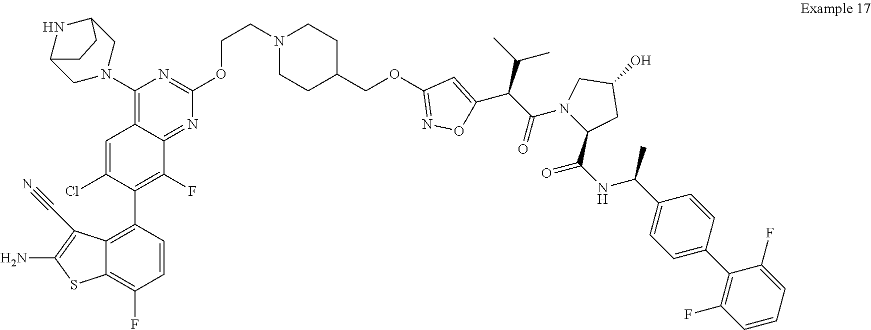 [SMILES: CC(C)[C@@H](C(=O)N1C[C@H](O)C[C@H]1C(=O)N[C@@H](C)c1ccc(-c2c(F)cccc2F)cc1)c1cc(OCC2CCN(CCOc3nc(N4CC5CCC(C4)N5)c4cc(Cl)c(-c5ccc(F)c6sc(N)c(C#N)c56)c(F)c4n3)CC2)no1]

To a solution of tert-butyl 3-(7-(2-amino-3-cyano-7-fluorobenzo[b]thiophen-4-yl)-6-chloro-2-(2-(4-(((5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy)methyl)piperidin-1-yl)ethoxy)-8-fluoroquinazolin-4-yl)-3,8-diazabicyclo[3.2.1]octane-8-carboxylate (68 mg, 0.05 mmol, 1 eq) in dichloromethane (1 mL) was added trifluoroacetic acid (1 mL). The mixture was stirred at 20° C. for 0.5 h. LCMS showed the starting material was consumed completely and one main peak with desired mass was detected. The reaction mixture was concentrated under reduced pressure to remove solvent. The residue was purified by prep-HPLC (column: Phenomenex luna C18 150*25 mm*10 um; mobile phase: [water (formic acid)-acetonitrile]; B %: 21%-41%, 2 min) to afford the desired product (27 mg, 0.02 mmol, 42% yield) as a white solid. LCMS (ESI, m/z): 1135.4 [M]⁺. ¹H NMR (400 MHz, DMSO-d₆) δ 8.45 (d, J=8.4 Hz, 1H), 8.18 (s, 1H), 8.12 (s, 1H), 7.85 (s, 1H), 7.52-7.41 (m, 2H), 7.38 (s, 4H), 7.29-7.12 (m, 5H), 6.07 (s, 1H), 5.12 (s, 1H), 4.96-4.90 (m, 1H), 4.43-4.38 (m, 3H), 4.31-4.25 (m, 3H), 3.98 (s, 1H), 3.64 (d, J=9.6 Hz, 2H), 3.57 (s, 6H), 2.99-2.93 (m, 2H), 2.72-2.68 (m, 2H), 2.04-1.97 (m, 3H), 1.71-1.59 (m, 8H), 1.39 (d, J=7.2 Hz, 3H), 1.27-1.22 (m, 2H), 0.95 (d, J=6.4 Hz, 3H), 0.78 (d, J=6.4 Hz, 3H).

Example 18: (2S,4R)-1-((2R)-2-(3-((1-(2-((7-(2-amino-3-cyano-7-fluorobenzo[b]thiophen-4-yl)-4-(3,8-diazabicyclo[3.2.1]octan-3-yl)-8-fluoro-6-(trifluoromethyl)quinazolin-2-yl)oxy)ethyl)piperidin-4-yl)methoxy)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide

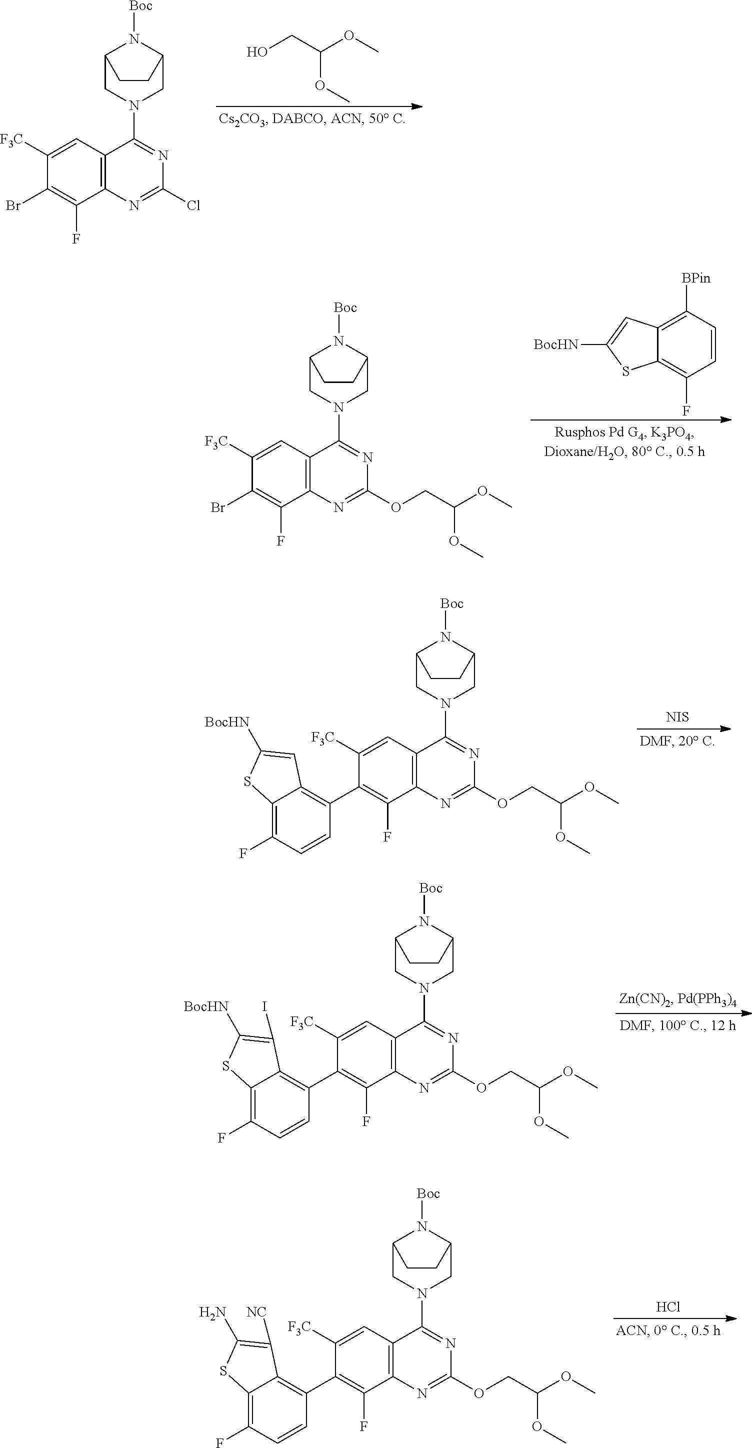

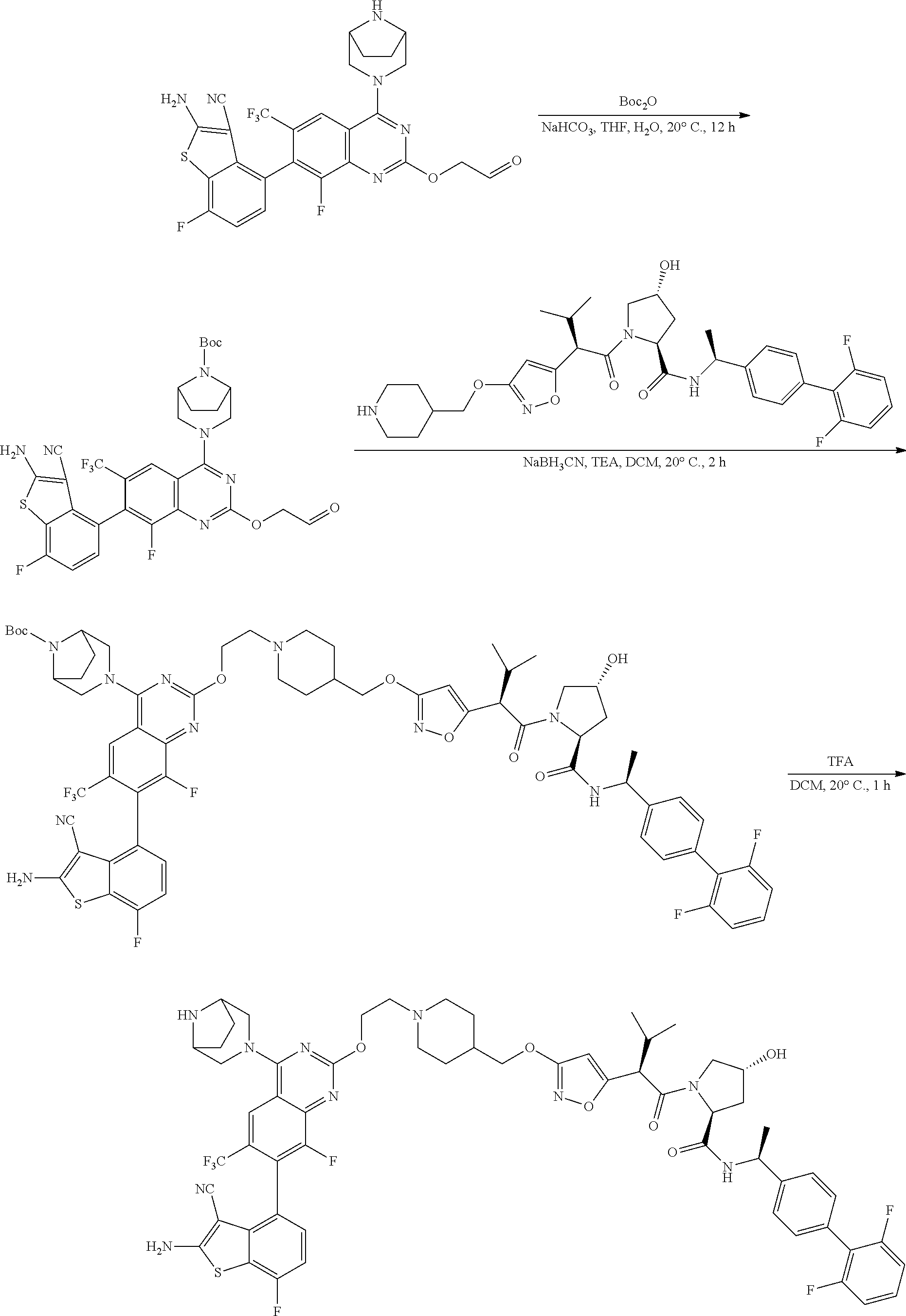

Step 1: tert-butyl-3-(7-bromo-2-(2,2-dimethoxyethoxy)-8-fluoro-6-(trifluoromethyl)quinazolin-4-yl)-3,8-diazabicyclo[3.2.1]octane-8-carboxylate

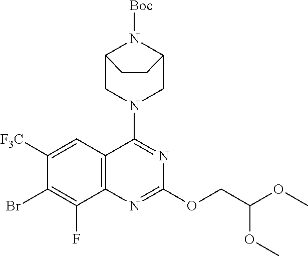 [SMILES: COC(COc1nc(N2CC3CCC(C2)N3C(=O)OC(C)(C)C)c2cc(C(F)(F)F)c(Br)c(F)c2n1)OC]

To a solution of tert-butyl 3-[7-bromo-2-chloro-8-fluoro-6-(trifluoromethyl)quinazolin-4-yl]-3,8-diazabicyclo[3.2.1]octane-8-carboxylate (10 g, 18.53 mmol, 1 eq) in acetonitrile (100 mL) was added 2,2-dimethoxyethanol (3.00 g, 28.27 mmol, 1.5 eq), 1,4-diazabicyclo[2.2.2]octane (200 mg, 1.78 mmol, 0.1 eq) and cesium carbonate (13.00 g, 39.90 mmol, 2.0 eq). The mixture was stirred at 50° C. for 3 h. TLC (Petroleum ether/Ethyl acetate=3/1) showed the tert-butyl 3-[7-bromo-2-chloro-8-fluoro-6-(trifluoromethyl)quinazolin-4-yl]-3,8-diazabicyclo[3.2.1]octane-8-carboxylate was consumed completely and a new spot was detected. The reaction mixture was filtered and concentrated under reduced pressure to give a residue. The residue was re-dissolved, partitioned between water (200 mL) and ethyl acetate (200 mL×3). The organic phase was separated, washed with brine (100 mL), dried over anhydrous sodium sulfate, filtered and concentrated under reduced pressure to give the crude product. The crude product was purified by silica gel chromatography (Petroleum ether/Ethyl acetate=1/0 to 4/1) to afford the product (8 g, 13.13 mmol, 710% yield) as a yellow solid.

Step 2: tert-butyl-3-(7-(2-((tert-butoxycarbonyl)amino)-7-fluorobenzo[b]thiophen-4-yl)-2-(2,2-dimethoxyethoxy)-8-fluoro-6-(trifluoromethyl)quinazolin-4-yl)-3,8-diazabicyclo[3.2.1]octane-8-carboxylate

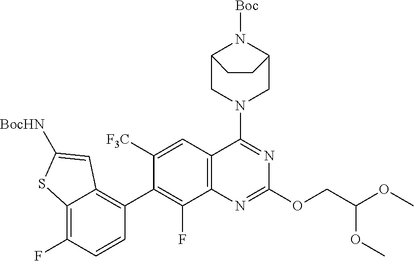 [SMILES: COC(COc1nc(N2CC3CCC(C2)N3C(=O)OC(C)(C)C)c2cc(C(F)(F)F)c(-c3ccc(F)c4sc(NC(=O)OC(C)(C)C)cc34)c(F)c2n1)OC]

To a solution of tert-butyl-3-(7-bromo-2-(2,2-dimethoxyethoxy)-8-fluoro-6-(trifluoromethyl)quinazolin-4-yl)-3,8-diazabicyclo[3.2.1]octane-8-carboxylate (3.5 g, 5.74 mmol, 1.0 eq) in dioxane (30 mL) and water (6 mL) was added tert-butyl N-[7-fluoro-4-(4,4,5,5-tetramethyl-1,3,2-dioxaborolan-2-yl)benzothiophen-2-yl]carbamate (2.26 g, 5.74 mmol, 1.0 eq), potassium phosphate (3.66 g, 17.22 mmol, 3.0 eq) and [2-(2-aminophenyl)phenyl]-methylsulfonyloxy-palladium; dicyclohexyl-[2-(2,6-diisopropoxyphenyl)phenyl]phosphane (800 mg, 0.96 mmol, 0.17 eq). The mixture was stirred at 80° C. for 0.5 h. LCMS showed the desired mass was detected. The reaction mixture was partitioned between water (100 mL) and ethyl acetate (100 mL×3). The organic phase was separated, washed with brine (20 mL), dried over anhydrous sodium sulfate, filtered and concentrated under reduced pressure to give a residue. The residue was purified by silica gel chromatography (Petroleum ether/Ethyl acetate=1/0 to 3/1) to afford the product (2.5 g, 3.14 mmol, 54% yield) as a yellow solid. LCMS (ESI, m/z): 796.1 [M+H]⁺. ¹H NMR (400 MHz, DMSO) δ 10.92-10.73 (m, 1H), 8.22 (s, 1H), 7.30-7.24 (m, 1H), 7.22-7.15 (m, 1H), 6.30 (d, J=2.8 Hz, 1H), 4.74 (d, J=5.2 Hz, 1H), 4.51 (d, J=12.4 Hz, 1H), 4.36 (d, J=3.6 Hz, 2H), 4.29 (br s, 2H), 3.93 (s, 1H), 3.70 (d, J=12.4 Hz, 1H), 3.60 (d, J=12.4 Hz, 1H), 3.35 (s, 6H), 1.83 (b s, 2H), 1.76-1.65 (m, 2H), 1.47 (s, 9H), 1.07 (s, 9H).

Step 3: tert-butyl-3-(7-(2-((tert-butoxycarbonyl)amino)-7-fluoro-3-iodobenzo[b]thiophen-4-yl)-2-(2,2-dimethoxyethoxy)-8-fluoro-6-(trifluoromethyl)quinazolin-4-yl)-3,8-diazabicyclo[3.2.1]octane-8-carboxylate

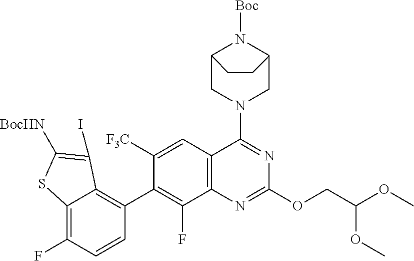 [SMILES: COC(COc1nc(N2CC3CCC(C2)N3C(=O)OC(C)(C)C)c2cc(C(F)(F)F)c(-c3ccc(F)c4sc(NC(=O)OC(C)(C)C)c(I)c34)c(F)c2n1)OC]

To a solution of tert-butyl-3-(7-(2-((tert-butoxycarbonyl)amino)-7-fluorobenzo[b]thiophen-4-yl)-2-(2,2-dimethoxyethoxy)-8-fluoro-6-(trifluoromethyl)quinazolin-4-yl)-3,8-diazabicyclo[3.2.1]octane-8-carboxylate (4.5 g, 5.65 mmol, 1.0 eq) in N,N-dimethylformamide (40 mL) was added N-iodosuccinimide (2.5 g, 11.11 mmol, 2.0 eq). The mixture was stirred at 20° C. for 1 h. LCMS showed the desired mass was detected. The reaction mixture was partitioned between water (200 mL) and ethyl acetate (200 mL×3). The organic phase was separated, washed with brine (20 mL), dried over anhydrous sodium sulfate, filtered and concentrated under reduced pressure to give a residue. The residue was purified by silica gel column chromatography (Petroleum ether/Ethyl acetate=10/1) to afford the desired product (4 g, 4.34 mmol, 76% yield) as a yellow solid. LCMS (ESI, m/z): 921.7 [M]⁺.

Step 4: tert-butyl-3-(7-(2-amino-3-cyano-7-fluorobenzo[b]thiophen-4-yl)-2-(2,2-dimethoxyethoxy)-8-fluoro-6-(trifluoromethyl)quinazolin-4-yl)-3,8-diazabicyclo[3.2.1]octane-8-carboxylate

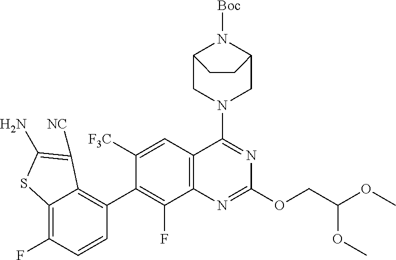 [SMILES: COC(COc1nc(N2CC3CCC(C2)N3C(=O)OC(C)(C)C)c2cc(C(F)(F)F)c(-c3ccc(F)c4sc(N)c(C#N)c34)c(F)c2n1)OC]

To a solution of tert-butyl-3-(7-(2-((tert-butoxycarbonyl)amino)-7-fluoro-3-iodobenzo[b]thiophen-4-yl)-2-(2,2-dimethoxyethoxy)-8-fluoro-6-(trifluoromethyl)quinazolin-4-yl)-3,8-diazabicyclo[3.2.1]octane-8-carboxylate (300 mg, 0.33 mmol, 1 eq) in N,N-dimethylformamide (5 mL) was added zinc cyanide (382 mg, 3.25 mmol, 10 eq) and tetrakis (triphenylphosphine) palladium(0) (75 mg, 0.06 mmol, 0.1 eq). The mixture was stirred at 100° C. for 12 h. LCMS showed the desired mass was detected. The mixture was filtered, diluted with water (20 mL), extracted with ethyl acetate (20 mL×3), washed with brine (20 mL), dried over anhydrous sodium sulfate, filtered and concentrated under reduced pressure to give a residue. The residue was purified by pre-TLC (7% methanol in dichloromethane) to afford the product (100 mg, 0.14 mmol, 42% yield) as a yellow solid. LCMS (ESI, m/z): 721.0 [M+H]⁺. ¹H NMR (400 MHz, CDCl₃) δ 8.03 (s, 1H), 7.24 (dd, J=5.2, 8.4 Hz, 1H), 7.03 (t, J=8.8 Hz, 1H), 5.39 (s, 2H), 4.85 (t, J=5.2 Hz, 1H), 4.51 (d, J=5.2 Hz, 3H), 4.45-4.29 (m, 3H), 3.81-3.56 (m, 2H), 3.51 (s, 2H), 3.48 (s, 6H), 1.96 (d, J=5.6 Hz, 2H), 1.54 (s, 9H).

Step 5: 4-(4-(3,8-diazabicyclo[3.2.1]octan-3-yl)-8-fluoro-2-(2-oxoethoxy)-6-(trifluoromethyl)quinazolin-7-yl)-2-amino-7-fluorobenzo[b]thiophene-3-carbonitrile

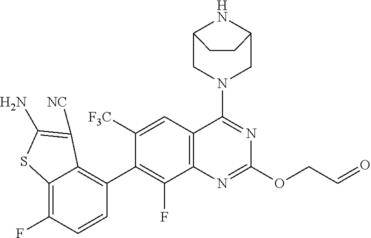 [SMILES: N#Cc1c(N)sc2c(F)ccc(-c3c(C(F)(F)F)cc4c(N5CC6CCC(C5)N6)nc(OCC=O)nc4c3F)c12]

To a solution of tert-butyl-3-(7-(2-amino-3-cyano-7-fluorobenzo[b]thiophen-4-yl)-2-(2,2-dimethoxyethoxy)-8-fluoro-6-(trifluoromethyl)quinazolin-4-yl)-3,8-diazabicyclo[3.2.1]octane-8-carboxylate (400 mg, 0.56 mmol, 1 eq) in acetonitrile (2 mL) was added hydrochloric acid (12 M, 0.5 mL, 10 eq). The mixture was stirred at 20° C. for 0.5 h. TLC (Petroleum ether/Ethyl acetate=1/1) showed the reactant was consumed completely and a new spot was detected. The mixture was concentrated under reduced pressure to afford the crude product (320 mg, crude) as a yellow oil, which was used into next step directly.

Step 6: tert-butyl--3-(7-(2-amino-3-cyano-7-fluorobenzo[b]thiophen-4-yl)-8-fluoro-2-(2-oxoethoxy)-6-(trifluoromethyl)quinazolin-4-yl)-3,8-diazabicyclo[3.2.1]octane-8-carboxylate

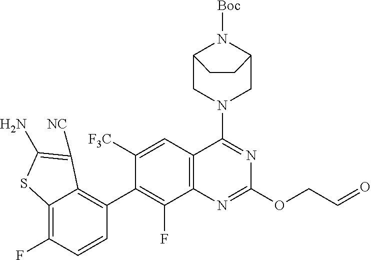 [SMILES: CC(C)(C)OC(=O)N1C2CCC1CN(c1nc(OCC=O)nc3c(F)c(-c4ccc(F)c5sc(N)c(C#N)c45)c(C(F)(F)F)cc13)C2]

To a solution of 4-(4-(3,8-diazabicyclo[3.2.1]octan-3-yl)-8-fluoro-2-(2-oxoethoxy)-6-(trifluoromethyl)quinazolin-7-yl)-2-amino-7-fluorobenzo[b]thiophene-3-carbonitrile (320 mg, 0.56 mmol, 1 eq) in tetrahydrofuran (5 mL) and water (1 mL) was added di-tert-butyl dicarbonate (304 mg, 1.39 mmol, 2.5 eq) and sodium bicarbonate (468 mg, 5.57 mmol, 10 eq). The mixture was stirred at 20° C. for 12 h. LCMS showed the desired mass was detected. The mixture was diluted with water (20 mL), extracted with ethyl acetate (20 mL×3), washed with brine (20 mL), dried over anhydrous sodium sulfate, filtered and concentrated under reduced pressure to give a residue. The residue was purified by pre-TLC (7% methanol in dichloromethane) to afford the product (240 mg, 0.36 mmol, 63% yield) as a yellow solid. LCMS (ESI, m/z): 675.2 [M+H]⁺.

Step 7: tert-butyl 3-(7-(2-amino-3-cyano-7-fluorobenzo[b]thiophen-4-yl)-2-(2-(4-(((5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy)methyl)piperidin-1-yl)ethoxy)-8-fluoro-6-(trifluoromethyl)quinazolin-4-yl)-3,8-diazabicyclo[3.2.1]octane-8-carboxylate

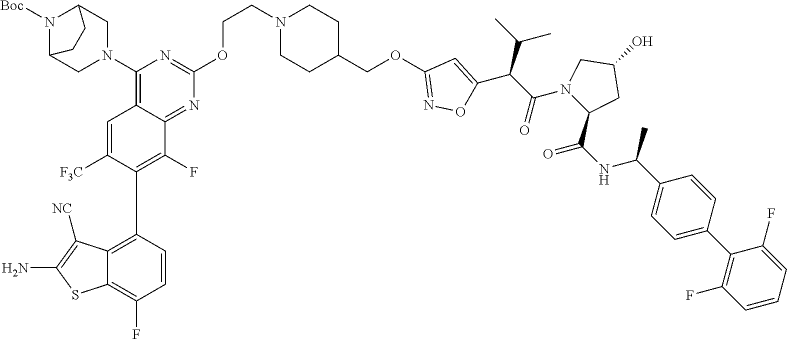 [SMILES: CC(C)[C@@H](C(=O)N1C[C@H](O)C[C@H]1C(=O)N[C@@H](C)c1ccc(-c2c(F)cccc2F)cc1)c1cc(OCC2CCN(CCOc3nc(N4CC5CCC(C4)N5C(=O)OC(C)(C)C)c4cc(C(F)(F)F)c(-c5ccc(F)c6sc(N)c(C#N)c56)c(F)c4n3)CC2)no1]

To a solution of tert-butyl--3-(7-(2-amino-3-cyano-7-fluorobenzo[b]thiophen-4-yl)-8-fluoro-2-(2-oxoethoxy)-6-(trifluoromethyl)quinazolin-4-yl)-3,8-diazabicyclo[3.2.1]octane-8-carboxylate and (2S,4R)—N-[(1S)-1-[4-(2,6-difluorophenyl)phenyl]ethyl]-4-hydroxy-1-[(2R)-3-methyl-2-[3-(4-piperidylmethoxy)isoxazol-5-yl]butanoyl]pyrrolidine-2-carboxamide (50 mg, 0.077 mmol, 1.3 eq, hydrochloride) in dichloromethane (2 mL) was added N,N-diisopropylethylamine (23 mg, 0.18 mmol, 3 eq). The mixture was stirred at 20° C. for 0.5 h, sodium triacetoxyborohydride (25 mg, 0.12 mmol, 2 eq) was added into the mixture and stirred at 20° C. for 11.5 h. LCMS showed the desired mass was detected. The mixture was concentrated under reduced pressure to get a residue. The residue was purified by pre-TLC (9% methanol in dichloromethane) to afford the desired product (25 mg, 0.02 mmol, 33% yield) as a yellow solid. LCMS (ESI, m/z): 1268.5 [M]⁺

Step 8: (2S,4R)-1-((2R)-2-(3-((1-(2-((7-(2-amino-3-cyano-7-fluorobenzo[b]thiophen-4-yl)-4-(3,8-diazabicyclo[3.2.1]octan-3-yl)-8-fluoro-6-(trifluoromethyl)quinazolin-2-yl)oxy)ethyl)piperidin-4-yl)methoxy)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide

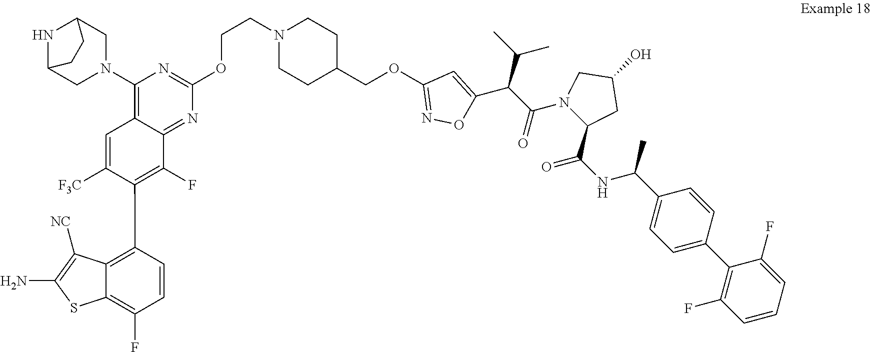 [SMILES: CC(C)[C@@H](C(=O)N1C[C@H](O)C[C@H]1C(=O)N[C@@H](C)c1ccc(-c2c(F)cccc2F)cc1)c1cc(OCC2CCN(CCOc3nc(N4CC5CCC(C4)N5)c4cc(C(F)(F)F)c(-c5ccc(F)c6sc(N)c(C#N)c56)c(F)c4n3)CC2)no1]

To a solution of tert-butyl 3-(7-(2-amino-3-cyano-7-fluorobenzo[b]thiophen-4-yl)-2-(2-(4-(((5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy)methyl)piperidin-1-yl)ethoxy)-8-fluoro-6-(trifluoromethyl)quinazolin-4-yl)-3,8-diazabicyclo[3.2.1]octane-8-carboxylate (25 mg, 0.02 mmol, 1 eq) in dichloromethane (3 mL) was added trifluoroacetic acid (1.0 mL). The mixture was stirred at 20° C. for 1 h. The reaction mixture was filtered and concentrated under reduced pressure to give a residue. The residue was purified by preparative high performance liquid chromatography (column: Phenomenex luna C18 150*25 mm*10 um; mobile phase: [water(FA)-ACN]; B %: 20%-40%, 2 min) to afford the product (12.61 mg, 0.011 mmol, 54% yield, 99% purity) as a white solid. LCMS (ESI, m/z): 1169.1 [M]⁺, ¹H NMR (400 MHz, DMSO) 8.47-8.37 (m, 1H), 8.26 (s, 1H), 8.13-8.00 (m, 3H), 7.47 (s, 1H), 7.41-7.36 (m, 4H), 7.27-7.18 (m, 3H), 7.16-7.11 (m, 1H), 6.07 (s, 1H), 5.00-4.88 (m, 1H), 4.44 (t, J=5.6 Hz, 2H), 4.38 (t, J=7.6 Hz, 1H), 4.34-4.22 (m, 3H), 4.03-3.92 (m, 2H), 3.59-3.54 (m, 4H), 2.98-2.93 (m, 2H), 2.29-2.19 (m, 3H), 2.08-1.99 (m, 4H), 1.78-1.53 (m, 10H), 1.39 (d, J=7.2 Hz, 3H), 1.33-1.21 (m, 3H), 0.99-0.93 (m, 3H), 0.82 (b s, 3H).

Example 19: (2S,4R)-1-((2S)-2-(3-(3-((2S)-2-((((7R)-7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)pyrrolidin-1-yl)propoxy)propanamido)-3,3-dimethylbutanoyl)-N—((S)-1-(2′,6′-difluoro-[1,1′-biphenyl]-4-yl)ethyl)-4-hydroxypyrrolidine-2-carboxamide

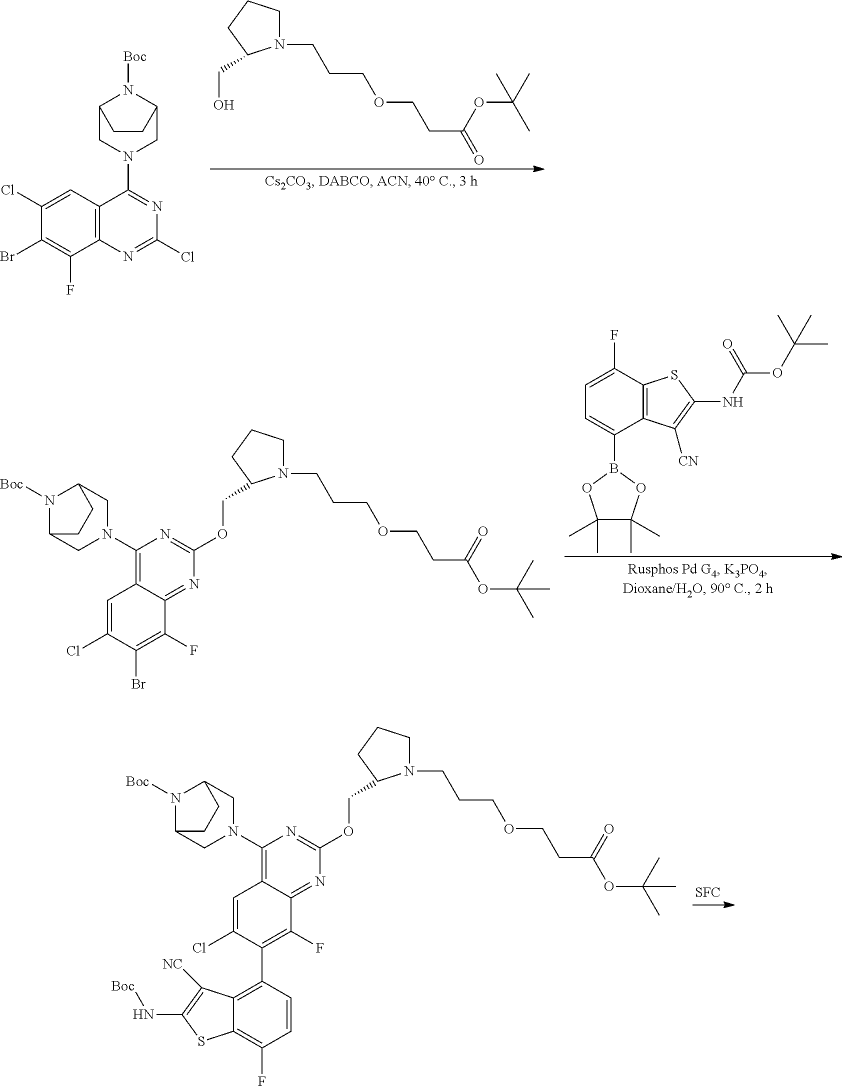

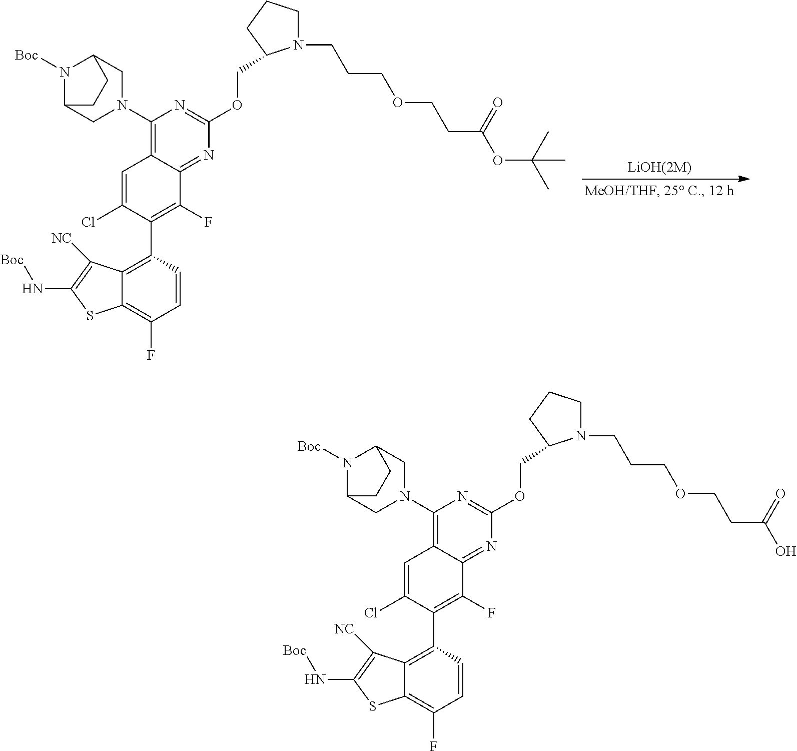

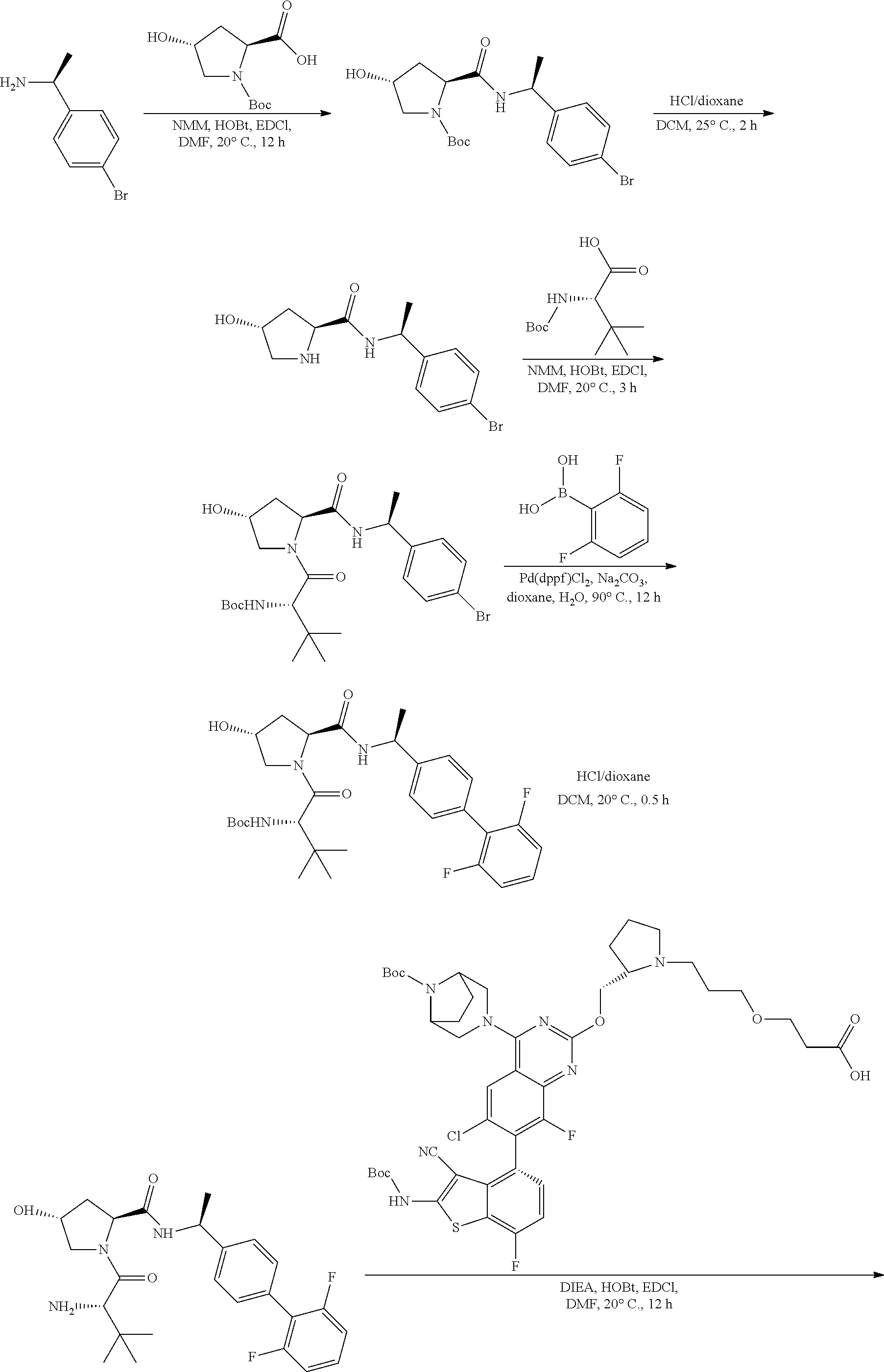

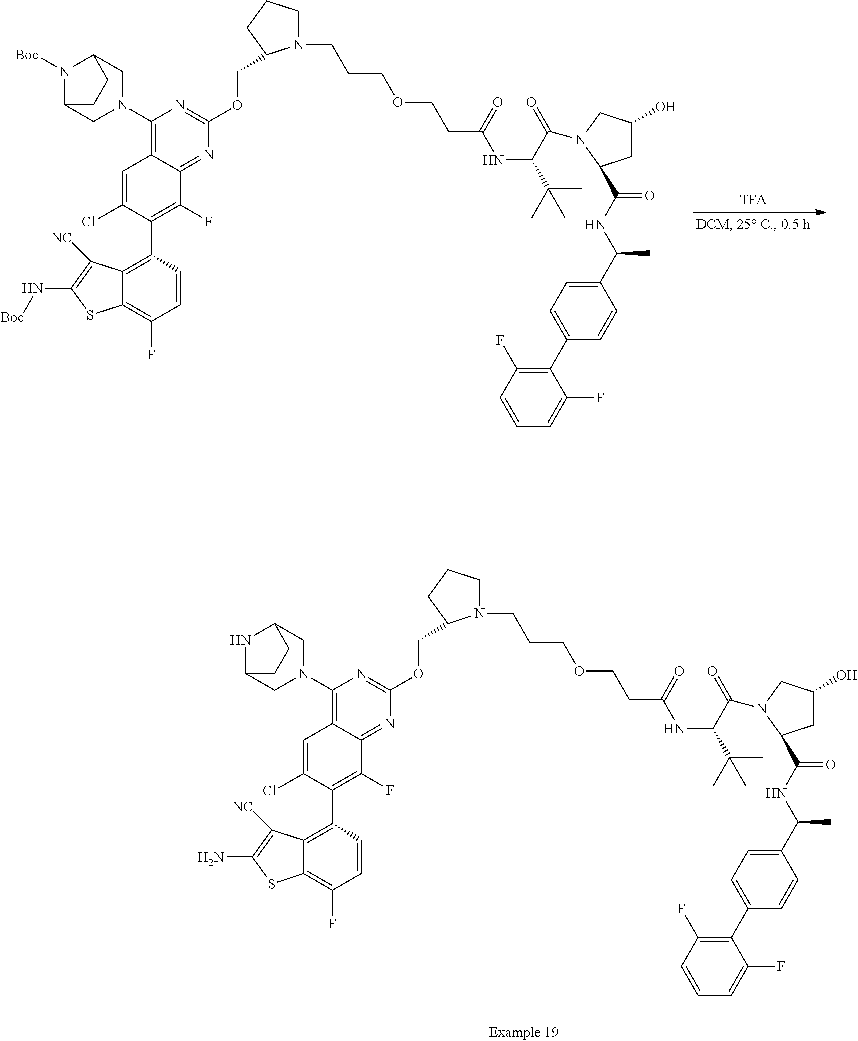

Step 1: tert-butyl 3-(7-bromo-2-(((S)-1-(3-(3-(tert-butoxy)-3-oxopropoxy)propyl)pyrrolidin-2-yl)methoxy)-6-chloro-8-fluoroquinazolin-4-yl)-3,8-diazabicyclo [3.2.1]octane-8-carboxylate

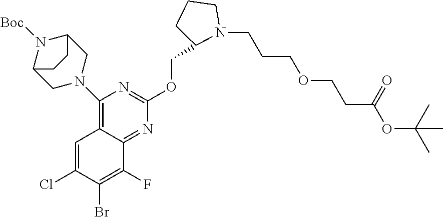

To a solution of tert-butyl-3-(7-bromo-6-chloro-2,8-difluoroquinazolin-4-yl)-3,8-diazabicyclo[3.2.1]octane-8-carboxylate (1 g, 2.04 mmol, 1 eq) and tert-butyl (S)-3-(3-(2-(hydroxymethyl)pyrrolidin-1-yl)propoxy)propanoate (1.17 g, 4.08 mmol, 2 eq) in acetonitrile (10 mL) was added cesium carbonate (1.33 g, 4.08 mmol, 2 eq) and 1,4-diazabicyclo [2.2.2]octane (22 mg, 0.20 mmol, 0.1 eq). The mixture was stirred at 40° C. for 3 h. Water (20 mL) was added before the mixture was extracted by ethyl acetate (20 mL×3). The combined organic layers were washed with brine (20 mL×3), dried over anhydrous sodium sulfate, filtered and concentrated under reduced pressure to afford a residue. The residue was purified by preparative high performance liquid chromatography (column: Phenomenex Synergi Max-RP 250*50 mm*10 um; mobile phase: [water(FA)-ACN]; B %: 35%-65%, 21 min) to give the product (529 mg, 0.63 mmol, 31% yield, 91% purity) as a yellow oil. LCMS (ESI, m/z): 757.9, 759.9 [M+H]⁺, ¹H NMR (400 MHz, CDCl₃) δ 8.42-8.39 (m, 1H), 4.74 (d, J=4.8, 11.2 Hz, 1H), 4.39 (s, 2H), 4.37-4.31 (m, 4H), 3.65 (t, J=6.4 Hz, 4H), 3.56-3.48 (m, 6H), 3.33-3.21 (m, 6H), 2.79 (d, J=3.2 Hz, 1H), 2.62 (d, J=9.6 Hz, 1H), 2.20-2.10 (m, 2H), 2.02-1.92 (m, 9H), 1.77 (d, J=3.2 Hz, 2H), 1.44 (s, 9H).

Step 2: tert-butyl 3-(2-(((S)-1-(3-(3-(tert-butoxy)-3-oxopropoxy)propyl)pyrrolidin-2-yl)methoxy)-7-(2-((tert-butoxycarbonyl)amino)-3-cyano-7-fluorobenzo[b]thiophen-4-yl)-6-chloro-8-fluoroquinazolin-4-yl)-3,8-diazabicyclo[3.2.1]octane-8-carboxylate

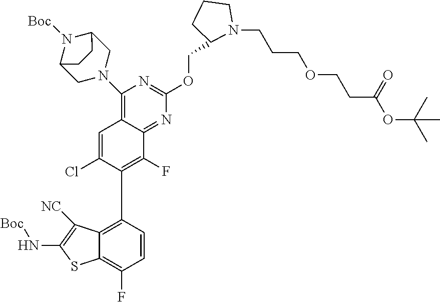

A mixture of tert-butyl 3-(7-bromo-2-(((S)-1-(3-(3-(tert-butoxy)-3-oxopropoxy)propyl)pyrrolidin-2-yl)methoxy)-6-chloro-8-fluoroquinazolin-4-yl)-3,8-diazabicyclo[3.2.1]octane-8-carboxylate (879 mg, 1.16 mmol, 1 eq), tert-butyl (3-cyano-7-fluoro-4-(4,4,5,5-tetramethyl-1,3,2-dioxaborolan-2-yl)benzo[b]thiophen-2-yl)carbamate (728 mg, 1.74 mmol, 1.5 eq), sodium carbonate (369 mg, 3.48 mmol, 3 eq) in dioxane (5 mL) and water (1 mL) was degassed and purged with nitrogen for 3 times. Then ditert-butyl(cyclopentyl)phosphane; dichloropalladium; iron (75 mg, 0.12 mmol, 0.1 eq) was added, and the mixture was stirred at 90° C. for 2 h in nitrogen atmosphere. Water (20 mL) was added before the mixture was extracted by ethyl acetate (20 mL×3). The combined organic layers were washed with brine (20 mL×3), dried over anhydrous sodium sulfate, filtered and concentrated under reduced pressure to afford a residue. The residue was purified by preparative high performance liquid chromatography (column: Phenomenex luna C18 250*50 mm*15 um; mobile phase: [water (FA)-ACN]; B %: 47%-77%, 10 min) to give the product (113 mg, 0.11 mmol, 9% yield, 94% purity) as a yellow solid. ¹H NMR (400 MHz, CDCl₃) δ 7.74 (s, 1H), 7.31 (d, J=4.4, 8.4 Hz, 1H), 7.16 (t, J=8.4 Hz, 1H), 4.55-4.27 (m, 5H), 4.26-4.06 (m, 3H), 3.71-3.55 (m, 4H), 3.55-3.43 (m, 2H), 3.21-3.09 (m, 1H), 3.02-2.86 (m, 1H), 2.45 (t, J=5.6 Hz, 2H), 2.05 (s, 3H), 2.00-1.91 (m, 3H), 1.90-1.72 (m, 6H), 1.57 (s, 9H), 1.53 (s, 9H), 1.43 (s, 9H).

Step 3: tert-butyl 3-((R)-2-(((S)-1-(3-(3-(tert-butoxy)-3-oxopropoxy)propyl)pyrrolidin-2-yl)methoxy)-7-(2-((tert-butoxycarbonyl)amino)-3-cyano-7-fluorobenzo[b]thiophen-4-yl)-6-chloro-8-fluoroquinazolin-4-yl)-3,8-diazabicyclo[3.2.1]octane-8-carboxylate

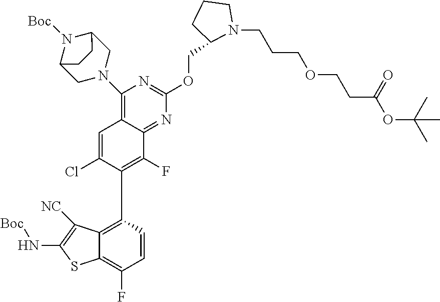

The mixture of atropisomers tert-butyl 3-(2-(((S)-1-(3-(3-(tert-butoxy)-3-oxopropoxy)propyl)pyrrolidin-2-yl)methoxy)-7-(2-((tert-butoxycarbonyl)amino)-3-cyano-7-fluorobenzo[b]thiophen-4-yl)-6-chloro-8-fluoroquinazolin-4-yl)-3,8-diazabicyclo[3.2.1]octane-8-carboxylate (7.70 g, 7.95 mmol, 1.00 eq) was separated by SFC (column: DAICEL CHIRALPAK IE (50*250 mm, 10 um); mobile phase: [Hexane-EtOH(0.1% FA)]; B %: 80%-80%, 15 min), and the second eluent (t_{R}=6.098 min) was identified as the desired atropisomer tert-butyl 3-((R)-2-(((S)-1-(3-(3-(tert-butoxy)-3-oxopropoxy)propyl)pyrrolidin-2-yl)methoxy)-7-(2-((tert-butoxycarbonyl)amino)-3-cyano-7-fluorobenzo[b]thiophen-4-yl)-6-chloro-8-fluoroquinazolin-4-yl)-3,8-diazabicyclo[3.2.1]octane-8-carboxylate. The product (3.20 g, 3.30 mmol, 41% yield) was obtained as a yellow solid. LCMS (ESI, m/z): 968.5 [M+1]⁺.

Step 4: 3-(3-((2S)-2-((((7R)-4-(8-(tert-butoxycarbonyl)-3,8-diazabicyclo[3.2.1]octan-3-yl)-7-(2-((tert-butoxycarbonyl)amino)-3-cyano-7-fluorobenzo[b]thiophen-4-yl)-6-chloro-8-fluoroquinazolin-2-yl)oxy)methyl)pyrrolidin-1-yl)propoxy)propanoic acid

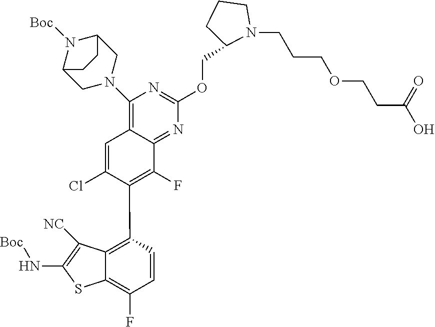

To a solution of tert-butyl 3-((R)-2-(((S)-1-(3-(3-(tert-butoxy)-3-oxopropoxy)propyl)pyrrolidin-2-yl)methoxy)-7-(2-((tert-butoxycarbonyl)amino)-3-cyano-7-fluorobenzo[b]thiophen-4-yl)-6-chloro-8-fluoroquinazolin-4-yl)-3,8-diazabicyclo[3.2.1]octane-8-carboxylate (3.20 g, 3.30 mmol, 1.00 eq) in tetrahydrofuran (30 mL), water (30 mL) was added lithium hydroxide monohydrate (2 M, 2.77 g, 66.08 mmol, 20.00 eq). The mixture was stirred at 25° C. for 12 h. The reaction mixture was concentrated in reduced pressure to give a residue, then pH of the mixture was adjusted to 6-7 by the addition of aqueous hydrogen chloride (0.5 M). The mixture was extracted with ethyl acetate (100 mL×3). The combined organic layers were washed with brine (50 mL×2), dried over anhydrous sodium sulfate, filtered and the filtrate was concentrated under reduced pressure to afford the crude product (3.20 g, 3.30 mmol, 99% yield, 94% purity) as a yellow solid, which was used into next step directly. LCMS (ESI, m/z): 912.4 [M+1]⁺.

Step 5: tert-butyl (2S,4R)-2-(((S)-1-(4-bromophenyl)ethyl)carbamoyl)-4-hydroxypyrrolidine-1-carboxylate

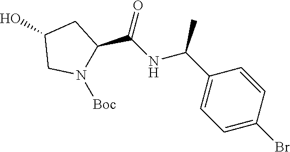 [SMILES: C[C@H](NC(=O)[C@@H]1C[C@@H](O)CN1C(=O)OC(C)(C)C)c1ccc(Br)cc1]

To a solution of (S)-1-(4-bromophenyl)ethan-1-amine (30 g, 149.94 mmol, 1 eq) and (2S,4R)-1-(tert-butoxycarbonyl)-4-hydroxypyrrolidine-2-carboxylic acid (34.67 g, 149.94 mmol, 1 eq) in N,N-dimethylformamide (300 mL) was added N-methyl morpholine (45.50 g, 449.83 mmol, 3 eq) and 1-hydroxybenzotriazole (41 g, 300 mmol, 2 eq). The mixture was stirred at 20° C. for 30 min. Then N-(3-dimethylaminopropyl)-N-ethylcarbodiimide hydrochloride (57 g, 300 mmol, 2 eq) was added to the mixture, and the mixture was stirred at 20° C. for 11.5 h. The mixture was diluted with water (1000 mL), extracted with ethyl acetate (300 mL×3). The organic layer was washed with brine (200 mL×3), dried over anhydrous sodium sulfate, filtered and concentrated under reduced pressure to get a residue. The residue was purified by silica gel column chromatography (petroleum ether/ethyl acetate=10/1 to 0/1) to afford the desired product (60 g, 145.17 mmol, crude) as a white solid. LCMS (ESI, m/z): 313.3, 315.3 [M+H]⁺.

Step 6: (2S,4R)—N—((S)-1-(4-bromophenyl)ethyl)-4-hydroxypyrrolidine-2-carboxamide

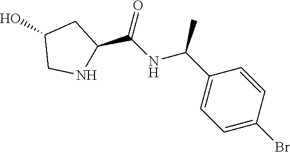 [SMILES: C[C@H](NC(=O)[C@@H]1C[C@@H](O)CN1)c1ccc(Br)cc1]

To a solution of tert-butyl (2S,4R)-2-(((S)-1-(4-bromophenyl)ethyl)carbamoyl)-4-hydroxypyrrolidine-1-carboxylate (20 g, 48.39 mmol, 1 eq) in dichloromethane (250 mL) was added hydrogen chloride/dioxane (4 M, 250 mL, 20 eq). The mixture was stirred at 25° C. for 2 h. The reaction mixture was filtered and concentrated under reduced pressure to afford the crude product (15 g, 47.53 mmol, 98% yield, 99% purity) as a white solid, which was used directly in the next step without further purification. LCMS (ESI, m/z): 315.1, 317.1 [M+H]⁺

Step 7: tert-butyl ((S)-1-((2S,4R)-2-(((S)-1-(4-bromophenyl)ethyl)carbamoyl)-4-hydroxypyrrolidin-1-yl)-3,3-dimethyl-1-oxobutan-2-yl)carbamate

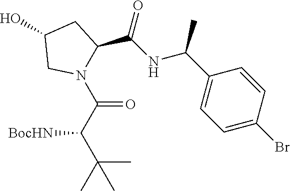 [SMILES: C[C@H](NC(=O)[C@@H]1C[C@@H](O)CN1C(=O)[C@@H](NC(=O)OC(C)(C)C)C(C)(C)C)c1ccc(Br)cc1]

To a solution of (2S,4R)—N—((S)-1-(4-bromophenyl)ethyl)-4-hydroxypyrrolidine-2-carboxamide (10 g, 31.93 mmol, 1 eq) and (S)-2-((tert-butoxycarbonyl)amino)-3,3-dimethylbutanoic acid (8.86 g, 38.32 mmol, 1.2 eq) in N,N-dimethylformamide (80 mL) was added 4-methylmorpholine (16.15 g, 159.65 mmol, 5 eq), 1-hydroxybenzotriazole (8.63 g, 63.86 mmol, 2 eq) and N-(3-dimethylaminopropyl)-N-ethylcarbodiimide hydrochloride (15.30 g, 79.82 mmol, 2.5 eq). The mixture was stirred at 20° C. for 3 h. Water (100 mL) was added before the mixture was extracted by ethyl acetate (100 mL×3). The combined organic layers were washed with brine (100 mL×3), dried over anhydrous sodium sulfate, filtered and concentrated under reduced pressure to give a residue. The residue was purified by column chromatography (petroleum/ethyl acetate=0/1) to afford the desired product (4.5 g, 8.55 mmol, 27% yield, 100% purity) as a white solid, LCMS (ESI, m/z): 528.2, 530.2 [M+H]+, ¹H NMR: (400 MHz, CDCl₃) δ: 7.57 (d, J=7.6 Hz, 1H), 7.45 (d, J=8.4 Hz, 2H), 7.19 (d, J=8.4 Hz, 2H), 5.24 (d, J=8.8 Hz, 1H), 4.99 (t, J=7.2 Hz, 1H), 4.72 (t, J=7.6 Hz, 1H), 4.48 (s, 1H), 4.20 (d, J=9.2 Hz, 1H), 3.57 (d, J=3.2, 11.6 Hz, 1H), 3.49-3.30 (m, 1H), 2.53-2.41 (m, 1H), 2.14-1.97 (m, 2H), 1.42 (s, 12H), 1.02 (s, 9H).

Step 8: tert-butyl ((S)-1-((2S,4R)-2-(((S)-1-(2′,6′-difluoro-[1,1′-biphenyl]-4-yl)ethyl)carbamoyl)-4-hydroxypyrrolidin-1-yl)-3,3-dimethyl-1-oxobutan-2-yl)carbamate

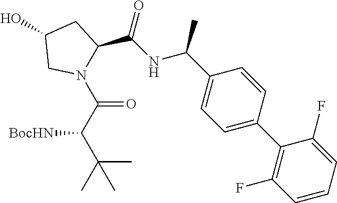 [SMILES: C[C@H](NC(=O)[C@@H]1C[C@@H](O)CN1C(=O)[C@@H](NC(=O)OC(C)(C)C)C(C)(C)C)c1ccc(-c2c(F)cccc2F)cc1]

To a solution of tert-butyl ((S)-1-((2S,4R)-2-(((S)-1-(4-bromophenyl)ethyl)carbamoyl)-4-hydroxypyrrolidin-1-yl)-3,3-dimethyl-1-oxobutan-2-yl)carbamate (1.00 g, 1.90 mmol, 1.00 eq) in dioxane (10 mL) and water (2 mL) was added (2,6-difluorophenyl)boronic acid (450 mg, 2.85 mmol, 1.50 eq), [1,1′-bis(diphenylphosphino)ferrocene]dichloropalladium(II) (139 mg, 0.20 mmol, 0.10 eq) and sodium carbonate (604 mg, 5.70 mmol, 3 eq). The mixture was stirred at 90° C. for 12 h under nitrogen atmosphere. The reaction mixture was partitioned between water (50 mL) and ethyl acetate (50 mL). The organic phase was separated, washed with brine (20 mL×3), dried over sodium sulfate, filtered and the filtrate was concentrated under reduced pressure to afford a residue. The residue was purified by silica gel chromatography (Petroleum ether/Ethyl acetate=1/0 to 1/1) to afford the product (600 mg, 1.07 mmol, 56% yield) as a yellow oil. LCMS (ESI, m/z): 560.4 [M+H]⁺

Step 9: (2S,4R)-1-((S)-2-amino-3,3-dimethylbutanoyl)-N—((S)-1-(2′,6′-difluoro-[1,1′-biphenyl]-4-yl)ethyl)-4-hydroxypyrrolidine-2-carboxamide

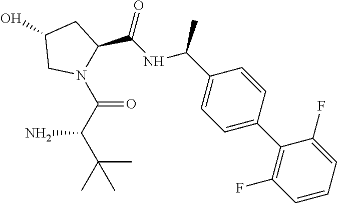 [SMILES: C[C@H](NC(=O)[C@@H]1C[C@@H](O)CN1C(=O)[C@@H](N)C(C)(C)C)c1ccc(-c2c(F)cccc2F)cc1]

To a solution of tert-butyl ((S)-1-((2S,4R)-2-(((S)-1-(2′,6′-difluoro-[1,1′-biphenyl]-4-yl)ethyl)carbamoyl)-4-hydroxypyrrolidin-l-yl)-3,3-dimethyl-1-oxobutan-2-yl)carbamate (400 mg, 0.7 mmol, 1 eq) in dichloromethane (5 mL) was added hydrochloride/dioxane (4 M, 1.0 mL, 6 eq). The mixture was stirred at 20° C. for 0.5 h. The reaction mixture was filtered and concentrated under reduced pressure to afford a residue. The residue was purified by prep-HPLC (column: Phenomenex luna C18 150*40 mm*15 um; mobile phase: [water(FA)-ACN]; B %: 15%-45%, 10 min) to afford the product (80 mg, 0.17 mmol, 24% yield) as a yellow oil. LCMS (ESI, m/z): 460.4 [M+H]⁺

Step 10: tert-butyl 3-((R)-7-(2-((tert-butoxycarbonyl)amino)-3-cyano-7-fluorobenzo[b]thiophen-4-yl)-6-chloro-2-(((S)-1-(3-(3-(((S)-1-((2S,4R)-2-(((S)-1-(2′,6′-difluoro-[1,1′-biphenyl]-4-yl)ethyl) carbamoyl)-4-hydroxypyrrolidin-1-yl)-3,3-dimethyl-1-oxobutan-2-yl)amino)-3-oxopropoxy) propyl)pyrrolidin-2-yl)methoxy)-8-fluoroquinazolin-4-yl)-3,8-diazabicyclo [3.2.1]octane-8-carboxylate

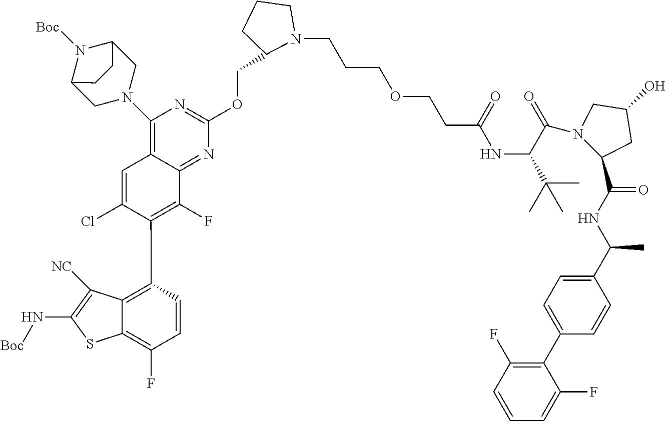

To a solution of (2S,4R)-1-((S)-2-amino-3,3-dimethylbutanoyl)-N—((S)-1-(2′,6′-difluoro-[1,1′-biphenyl]-4-yl)ethyl)-4-hydroxypyrrolidine-2-carboxamide (80 mg, 0.16 mmol, 1 eq, formate) in N,N-dimethylformamide (5 mL) was added 1-hydroxybenzotriazole (42 mg, 0.32 mmol, 2 eq), N,N-diisopropylethylamine (102 mg, 0.79 mmol, 5 eq), 3-(3-((2S)-2-((((7R)-4-(8-(tert-butoxycarbonyl)-3,8-diazabicyclo[3.2.1]octan-3-yl)-7-(2-((tert-butoxycarbonyl)amino)-3-cyano-7-fluorobenzo[b]thiophen-4-yl)-6-chloro-8-fluoroquinazolin-2-yl)oxy) methyl)pyrrolidin-1-yl)propoxy)propanoic acid (76 mg, 0.4 mmol, 2 eq). The mixture was stirred at 20° C. for 12 h. The reaction mixture was partitioned between water (20 mL) and ethyl acetate (20 mL). The organic phase was separated, washed with brine (20 mL×3), dried over anhydrous sodium sulfate, filtered and concentrated under reduced pressure to afford a residue. The residue was purified by prep-TLC (9% methanol in dichloromethane) to afford the product (138 mg, 0.10 mmol, 64% yield) was obtained as a yellow solid. LCMS (ESI, m/z): 1355.1 [M+H]⁺.

Step 11: (2S,4R)-1-((2S)-2-(3-(3-((2S)-2-((((7R)-7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)pyrrolidin-1-yl)propoxy)propanamido)-3,3-dimethylbutanoyl)-N—((S)-1-(2′,6′-difluoro-[1,1′-biphenyl]-4-yl)ethyl)-4-hydroxypyrrolidine-2-carboxamide (Example 19)

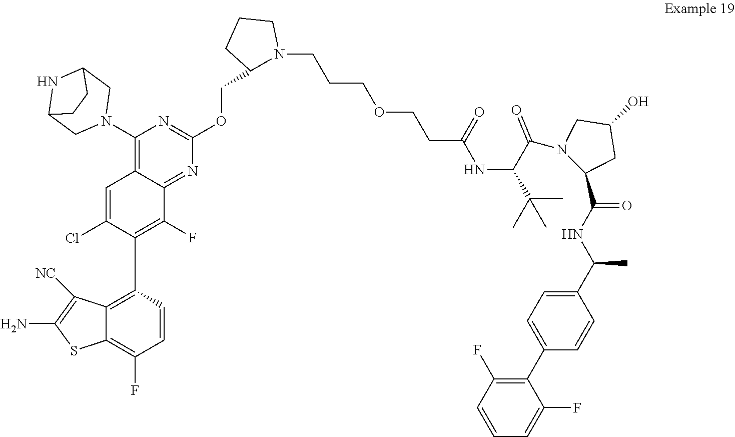

To a solution of tert-butyl 3-((R)-7-(2-((tert-butoxycarbonyl)amino)-3-cyano-7-fluorobenzo[b]thiophen-4-yl)-6-chloro-2-(((S)-1-(3-(3-(((S)-1-((2S,4R)-2-(((S)-1-(2′,6′-difluoro-[1,1′-biphenyl]-4-yl)ethyl) carbamoyl)-4-hydroxypyrrolidin-1-yl)-3,3-dimethyl-1-oxobutan-2-yl)amino)-3-oxopropoxy) propyl)pyrrolidin-2-yl)methoxy)-8-fluoroquinazolin-4-yl)-3,8-diazabicyclo[3.2.1]octane-8-carboxylate (135 mg, 0.10 mmol, 1 eq) in dichloromethane (3 mL) was added trifluoroacetic acid (3 mL). The mixture was stirred at 20° C. for 0.5 h. The reaction mixture was filtered and concentrated under reduced pressure to afford a residue. The residue was purified by preparative high performance liquid chromatography (column: Phenomenex Synergi C18 150*25 mm*10 um; mobile phase: [water(FA)-ACN]; B %: 14%-44%, 10 min) to give the product (52.19 mg, 0.01 mmol, 45% yield) as a yellow solid. LCMS (ESI, m/z): 1154.9 [M+H]⁺, ¹H NMR: (400 MHz, DMSO-d₆) δ: 8.44-8.33 (m, 1H), 8.21 (s, 2H), 8.11 (s, 2H), 7.90-7.77 (m, 2H), 7.52-7.35 (m, 5H), 7.32-7.08 (m, 4H), 4.94 (t, J=7.2 Hz, 1H), 4.52 (d, J=9.6 Hz, 1H), 4.43 (t, J=8.0 Hz, 1H), 4.37-4.25 (m, 4H), 4.09-4.03 (m, 1H), 3.62-3.52 (m, 11H), 3.06-3.03 (m, 1H), 2.93-2.80 (m, 2H), 2.30 (b s, 1H), 2.18 (b d, J=8.4 Hz, 1H), 2.01 (b d, J=0.8, 10.4 Hz, 1H), 1.92-1.80 (m, 2H), 1.74-1.54 (m, 10H), 1.39 (d, J=7.2 Hz, 3H), 0.91 (b d, J=3.2 Hz, 10H).

Example 20: (2S,4R)-1-((2S)-2-(3-(3-((2S)-2-((((7S)-7-(2-amino-3-cyano-7-fluorobenzo[b]thiophen-4-yl)-4-(3,8-diazabicyclo[3.2.1]octan-3-yl)-8-fluoro-6-(trifluoromethyl)quinazolin-2-yl)oxy)methyl)pyrrolidin-1-yl)propoxy)propanamido)-3,3-dimethylbutanoyl)-N—((S)-1-(2′,6′-difluoro-[1,1′-biphenyl]-4-yl)ethyl)-4-hydroxypyrrolidine-2-carboxamide

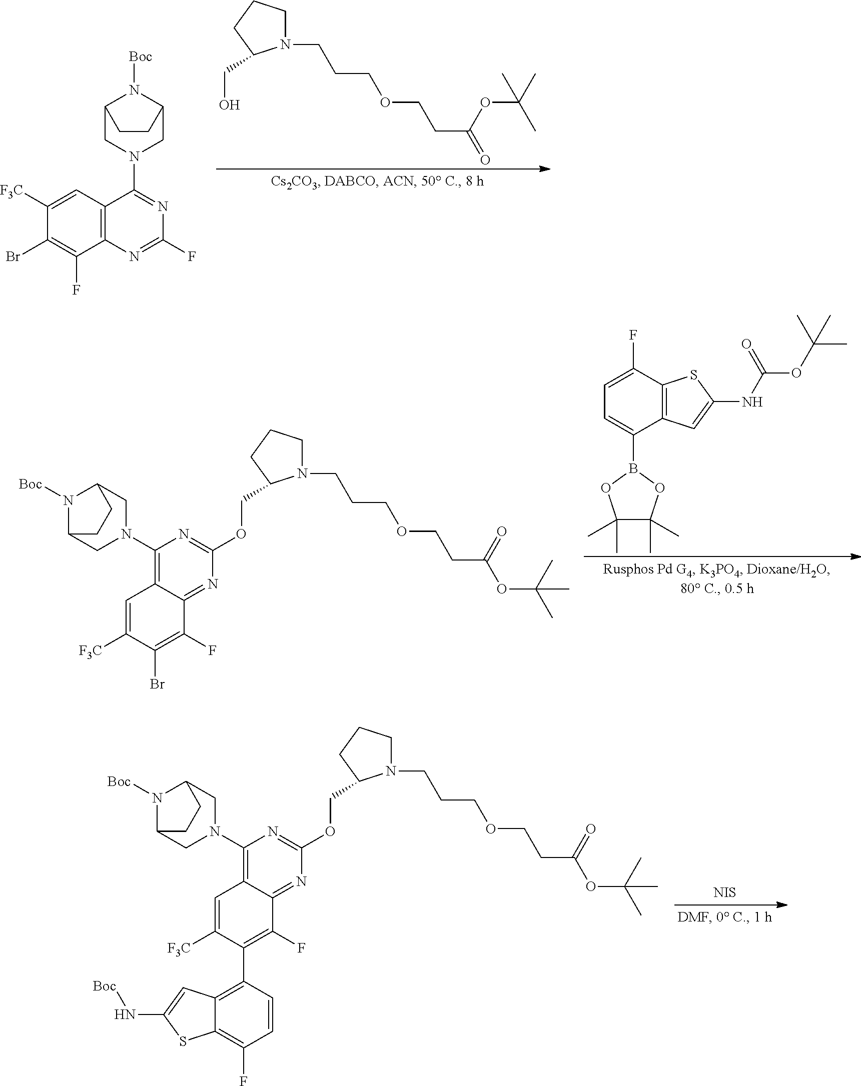

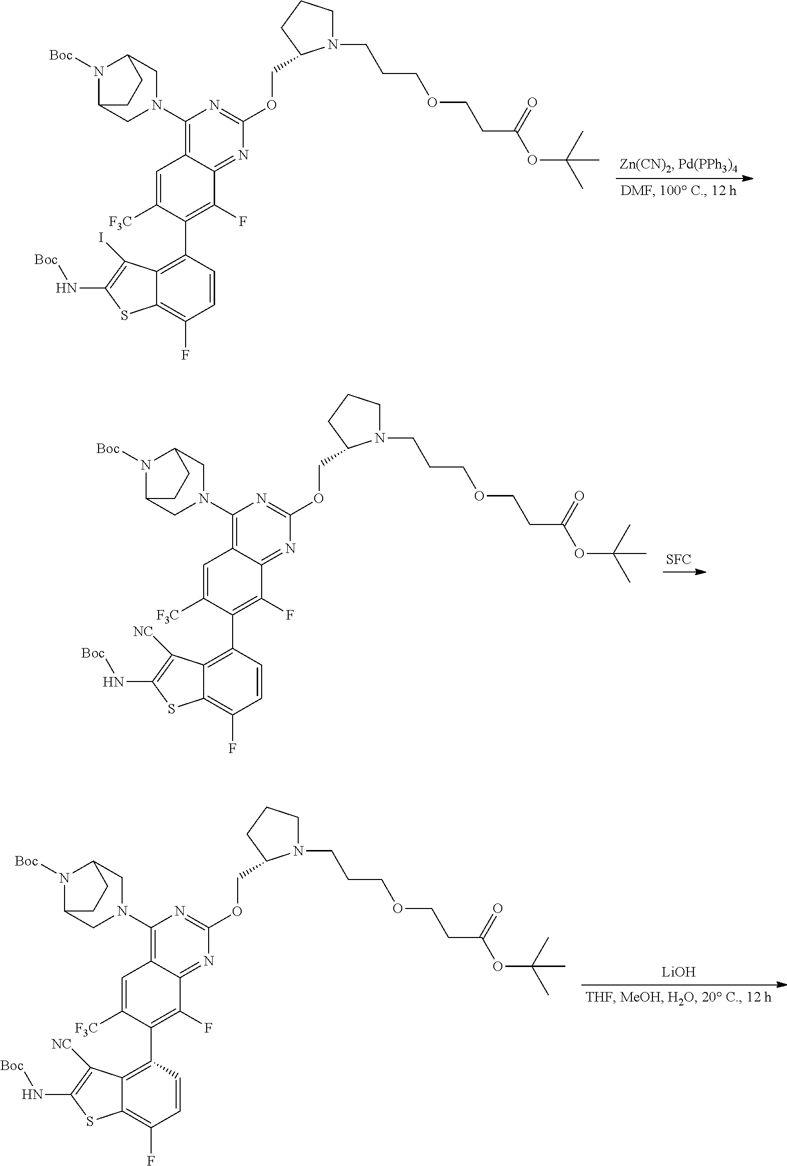

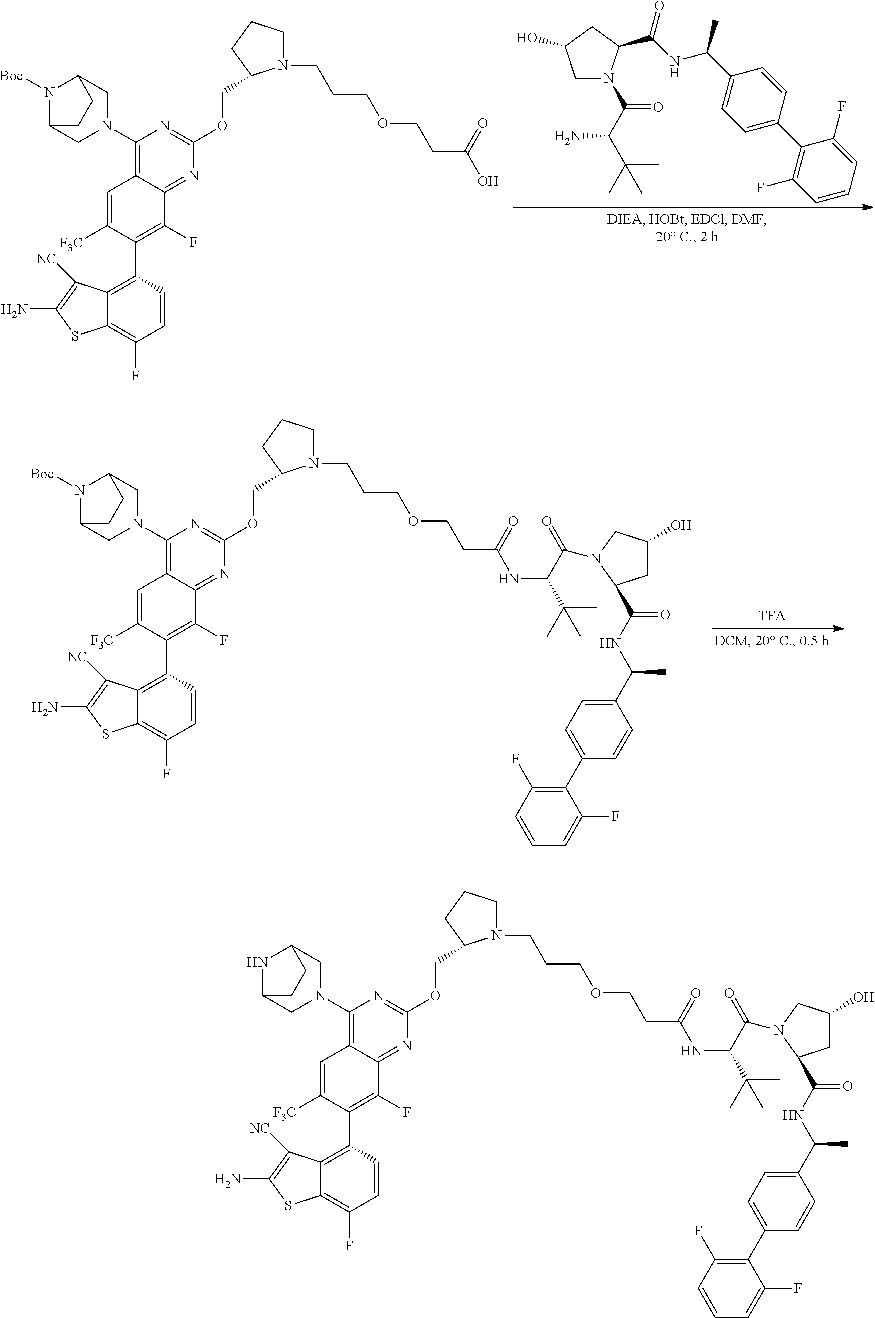

Step 1: tert-butyl 3-[7-bromo-2-[[(2S)-1-[3-(3-tert-butoxy-3-oxo-propoxy)propyl]pyrrolidin-2-yl]methoxy]-8-fluoro-6-(trifluoromethyl)quinazolin-4-yl]-3,8-diazabicyclo[3.2.1]octane-8-carboxylate

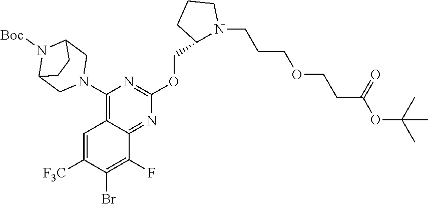 [SMILES: CC(C)(C)OC(=O)CCOCCCN1CCC[C@H]1COc1nc(N2CC3CCC(C2)N3C(=O)OC(C)(C)C)c2cc(C(F)(F)F)c(Br)c(F)c2n1]

To a solution of tert-butyl 3-[3-[(2S)-2-(hydroxymethyl)pyrrolidin-1-yl]propoxy]propanoate (3.30 g, 11.47 mmol, 2.00 eq) in acetonitrile (15 mL) was added tert-butyl 3-[7-bromo-2,8-difluoro-6-(trifluoromethyl)quinazolin-4-yl]-3,8-diazabicyclo[3.2.1]octane-8-carboxylate (3.00 g, 5.73 mmol, 1.00 eq), 1,4-diazabicyclo[2.2.2]octane (129 mg, 1.15 mmol, 0.1 mL, 0.20 eq) and cesium carbonate (3.74 g, 11.47 mmol, 2.00 eq) at 50° C. The mixture was stirred at 50° C. for 5 h. After the reaction was completed, the mixture was filtered, and the filtrate was concentrated under reduced pressure to give a residue. The residue was purified by preparative high performance liquid chromatography (column: Phenomenex luna C18 (250*70 mm, 10 um); mobile phase: [water(FA)-ACN]; B %: 35%-65%, 21 min) to afford the desired product (2.50 g, 3.16 mmol, 50% yield) as a light yellow gum. LCMS (ESI, m/z): 790.2, 792.2 [M+H]⁺.

Step 2: tert-butyl 3-[7-[2-(tert-butoxycarbonylamino)-7-fluoro-benzothiophen-4-yl]-2-[[(2S)-1-[3-(3-tert-butoxy-3-oxo-propoxy)propyl]pyrrolidin-2-yl]methoxy]-8-fluoro-6-(trifluoromethyl)quinazolin-4-yl]-3,8-diazabicyclo[3.2.1]octane-8-carboxylate

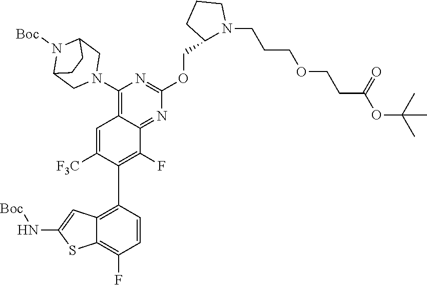 [SMILES: CC(C)(C)OC(=O)CCOCCCN1CCC[C@H]1COc1nc(N2CC3CCC(C2)N3C(=O)OC(C)(C)C)c2cc(C(F)(F)F)c(-c3ccc(F)c4sc(NC(=O)OC(C)(C)C)cc34)c(F)c2n1]

To a solution of tert-butyl 3-[7-bromo-2-[[(2S)-1-[3-(3-tert-butoxy-3-oxo-propoxy)propyl]pyrrolidin-2-yl]methoxy]-8-fluoro-6-(trifluoromethyl)quinazolin-4-yl]-3,8-diazabicyclo[3.2.1]octane-8-carboxylate (1.00 g, 1.26 mmol, 1.00 eq) in dioxane (20 mL) and water (4 mL) was added tert-butyl N-[7-fluoro-4-(4,4,5,5-tetramethyl-1,3,2-dioxaborolan-2-yl)benzothiophen-2-yl]carbamate (995 mg, 2.53 mmol, 2.00 eq), potassium phosphate (805 mg, 3.79 mmol, 3.00 eq) and RuPhos Pd G4 (212 mg, 0.25 mmol, 0.20 eq) at 80° C. under nitrogen. The mixture was stirred at 80° C. for 0.5 h. After the reaction was completed, the mixture was filtered and the filtrate was concentrated under reduced pressure to give a residue. The residue was purified by preparative high performance liquid chromatography (column: Phenomenex luna c18 250 mm*100 mm*10 um; mobile phase: [water(FA)-ACN]; B %: 50%-70%, 20 min) to afford the desired product (0.75 g, 0.76 mmol, 60% yield) as a light yellow solid. LCMS (ESI, m/z): 977.1 [M+H]⁺.

Step 3: tert-butyl 3-[7-[2-(tert-butoxycarbonylamino)-7-fluoro-3-iodo-benzothiophen-4-yl]-2-[[(2S)-1-[3-(3-tert-butoxy-3-oxo-propoxy)propyl]pyrrolidin-2-yl]methoxy]-8-fluoro-6-(trifluoromethyl)quinazolin-4-yl]-3,8-diazabicyclo[3.2.1]octane-8-carboxylate

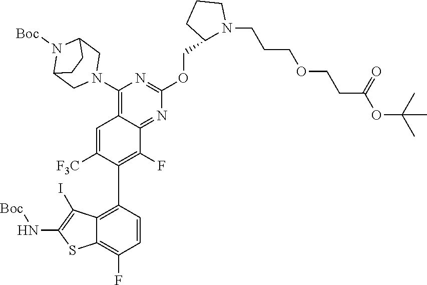 [SMILES: CC(C)(C)OC(=O)CCOCCCN1CCC[C@H]1COc1nc(N2CC3CCC(C2)N3C(=O)OC(C)(C)C)c2cc(C(F)(F)F)c(-c3ccc(F)c4sc(NC(=O)OC(C)(C)C)c(I)c34)c(F)c2n1]

To a solution of tert-butyl 3-[7-[2-(tert-butoxycarbonylamino)-7-fluoro-benzothiophen-4-yl]-2-[[(2S)-1-[3-(3-tert-butoxy-3-oxo-propoxy)propyl]pyrrolidin-2-yl]methoxy]-8-fluoro-6-(trifluoromethyl)quinazolin-4-yl]-3,8-diazabicyclo[3.2.1]octane-8-carboxylate (2.00 g, 2.05 mmol, 1.00 eq) in N,N-dimethyl formamide (30 mL) was added N-Iodosuccinimide (691 mg, 3.07 mmol, 1.50 eq) at 0° C. And the mixture was stirred at 0° C. for 2 h. After the reaction was completed, the mixture was filtered and the filtrate was purified by preparative high performance liquid chromatography(column: Phenomenex luna C18 250*80 mm*10 um; mobile phase: [water(FA)-ACN]; B %: 50%-80%, 20 min) to afford the desired product (1.43 g, 1.30 mmol, 63% yield) as a light yellow solid. LCMS (ESI, m/z): 1103.3 [M+H]⁺. ¹H NMR (400 MHz, DMSO) δ 9.44 (s, 1H), 8.21 (s, 1H), 7.40-7.25 (m, 2H), 4.84-4.61 (m, 2H), 4.55-4.37 (m, 2H), 4.31 (s, 2H), 3.91 (s, 1H), 3.74 (d, J=7.6 Hz, 1H), 3.63 (d, J=6.8 Hz, 2H), 3.53 (t, J=6.0 Hz, 2H), 3.34 (s, 2H), 3.15 (d, J=1.6 Hz, 2H), 2.39 (s, 2H), 2.30-2.18 (m, 1H), 2.06-1.58 (m, TOH), 1.49 (d, J=7.6 Hz, 18H), 1.36 (d, J=2.8 Hz, 9H).

Step 4: tert-butyl 3-[7-(2-amino-3-cyano-7-fluoro-benzothiophen-4-yl)-2-[[(2S)-1-[3-(3-tert-butoxy-3-oxo-propoxy)propyl]pyrrolidin-2-yl]methoxy]-8-fluoro-6-(trifluoromethyl)quinazolin-4-yl]-3,8-diazabicyclo[3.2.1]octane-8-carboxylate

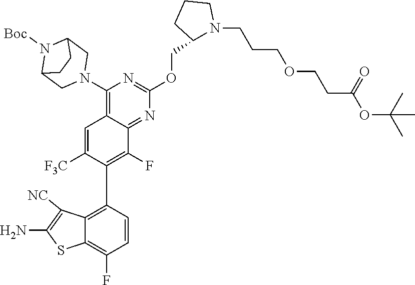 [SMILES: CC(C)(C)OC(=O)CCOCCCN1CCC[C@H]1COc1nc(N2CC3CCC(C2)N3C(=O)OC(C)(C)C)c2cc(C(F)(F)F)c(-c3ccc(F)c4sc(N)c(C#N)c34)c(F)c2n1]

To a solution of tert-butyl 3-[7-[2-(tert-butoxycarbonylamino)-7-fluoro-3-iodo-benzothiophen-4-yl]-2-[[(2S)-1-[3-(3-tert-butoxy-3-oxo-propoxy)propyl]pyrrolidin-2-yl]methoxy]-8-fluoro-6-(trifluoromethyl)quinazolin-4-yl]-3,8-diazabicyclo [3.2.1]octane-8-carboxylate (1.00 g, 0.91 mmol, 1.00 eq) in N,N-dimethyl formamide (16 mL) was added zinc cyanide (750 mg, 6.39 mmol, 0.4 mL, 7.04 eq), tetratriphenylphosphopalladium (250 mg, 0.22 mmol, 0.20 eq) at 100° C. under nitrogen and the mixture was stirred at 100° C. for 12 h. The mixture was diluted with water (100 mL) and extracted with ethyl acetate (60 mL×3). The combined organic layers were washed with water (60 mL), brine (60 mL), dried over sodium sulfate and then concentrated under reduced pressure to give a residue. The residue was purified by preparative high performance liquid chromatography (column: Phenomenex luna C18 150*25 mm*10 um; mobile phase: [water(FA)-ACN]; B %: 30%-60%, 2 min) to afford the desired product (70 mg, 0.08 mmol, 9% yield) as a white solid. LCMS (ESI, m/z): 902.3 [M+H]⁺.

Step 5: tert-butyl 3-((S)-7-(2-amino-3-cyano-7-fluorobenzo[b]thiophen-4-yl)-2-(((S)-1-(3-(3-(tert-butoxy)-3-oxopropoxy)propyl)pyrrolidin-2-yl)methoxy)-8-fluoro-6-(trifluoromethyl)quinazolin-4-yl)-3,8-diazabicyclo[3.2.1]octane-8-carboxylate

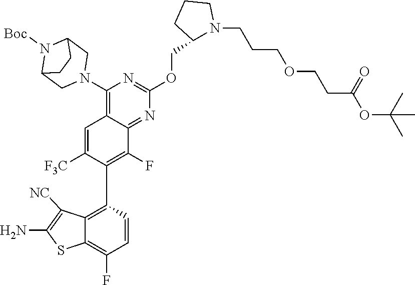

The mixture of atropisomers tert-butyl 3-((R)-7-(2-amino-3-cyano-7-fluorobenzo[b]thiophen-4-yl)-2-(((S)-1-(3-(3-(tert-butoxy)-3-oxopropoxy)propyl)pyrrolidin-2-yl)methoxy)-8-fluoro-6-(trifluoromethyl)quinazolin-4-yl)-3,8-diazabicyclo[3.2.1]octane-8-carboxylate and tert-butyl 3-((S)-7-(2-amino-3-cyano-7-fluorobenzo[b]thiophen-4-yl)-2-(((S)-1-(3-(3-(tert-butoxy)-3-oxopropoxy)propyl)pyrrolidin-2-yl)methoxy)-8-fluoro-6-(trifluoromethyl)quinazolin-4-yl)-3,8-diazabicyclo[3.2.1]octane-8-carboxylate (300 mg, 0.33 mmol, 1.00 eq) was separated by SFC (column: DAICEL CHIRALPAK IE(250 mm*30 mm, 10 um); mobile phase: [ACN/IPA(0.1% NH3H2O)]; B %: 60%-60%, 6.4 min), and the second eluent was identified as the desired atropisomer tert-butyl 3-((S)-7-(2-amino-3-cyano-7-fluorobenzo[b]thiophen-4-yl)-2-(((S)-1-(3-(3-(tert-butoxy)-3-oxopropoxy)propyl)pyrrolidin-2-yl)methoxy)-8-fluoro-6-(trifluoromethyl)quinazolin-4-yl)-3,8-diazabicyclo[3.2.1]octane-8-carboxylate (120 mg, 0.13 mmol, 40% yield) as a white solid.

Step 6: 3-(3-((2S)-2-((((7S)-7-(2-amino-3-cyano-7-fluorobenzo[b]thiophen-4-yl)-4-(8-(tert-butoxycarbonyl)-3,8-diazabicyclo[3.2.1]octan-3-yl)-8-fluoro-6-(trifluoromethyl)quinazolin-2-yl)oxy)methyl)pyrrolidin-1-yl)propoxy)propanoic acid

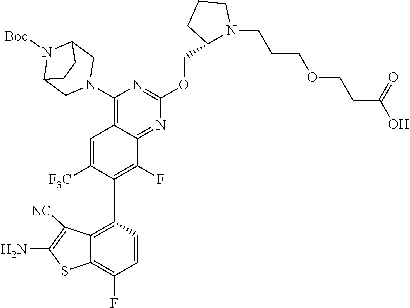

To a solution of tert-butyl 3-((S)-7-(2-amino-3-cyano-7-fluorobenzo[b]thiophen-4-yl)-2-(((S)-1-(3-(3-(tert-butoxy)-3-oxopropoxy)propyl)pyrrolidin-2-yl)methoxy)-8-fluoro-6-(trifluoromethyl)quinazolin-4-yl)-3,8-diazabicyclo[3.2.1]octane-8-carboxylate (58 mg, 0.06 mmol, 1.00 eq) in tetrahydrofuran (1 mL) and methanol (1 mL) was added a solution of lithium hydrate (50 mg, 1.19 mmol, 18.53 eq) in water (0.5 mL) at 20° C. The mixture was stirred at 20° C. for 12 h. The reaction mixture was concentrated under reduced pressure to give a residue. The residue was diluted with water (10 mL) and adjusted by aqueous hydrochloride (1M) to pH=4. The solution was extracted with ethyl acetate (20 mL×3) and dried over sodium sulfate and then concentrated under reduced pressure to give the desired product (50 mg, 0.06 mmol, 92% yield) as a light yellow solid. LCMS (ESI, m/z): 846.2 [M+H]⁺.

Step 7: tert-butyl 3-((S)-7-(2-amino-3-cyano-7-fluorobenzo[b]thiophen-4-yl)-2-(2-((S)-1-(3-(3-(((S)-1-((2S,4R)-2-(((S)-1-(2′,6′-difluoro-[1,1′-biphenyl]-4-yl)ethyl)carbamoyl)-4-hydroxypyrrolidin-1-yl)-3,3-dimethyl-1-oxobutan-2-yl)amino)-3-oxopropoxy)propyl)pyrrolidin-2-yl)ethyl)-8-fluoro-6-(trifluoromethyl)quinazolin-4-yl)-3,8-diazabicyclo[3.2.1]octane-8-carboxylate

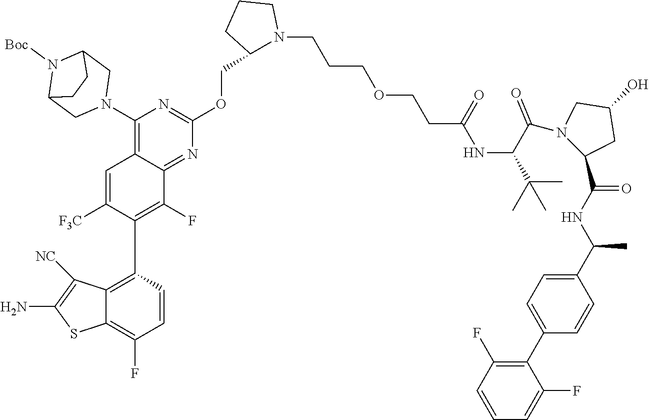

To a solution of 3-(3-((2S)-2-((((7S)-7-(2-amino-3-cyano-7-fluorobenzo[b]thiophen-4-yl)-4-(8-(tert-butoxycarbonyl)-3,8-diazabicyclo[3.2.1]octan-3-yl)-8-fluoro-6-(trifluoromethyl)quinazolin-2-yl)oxy)methyl)pyrrolidin-1-yl)propoxy)propanoic acid (50 mg, 0.06 mmol, 1.00 eq) in N,N-dimethyl formamide (1 mL) was added 1H-benzo[d][1,2,3]triazol-1-ol (16 mg, 0.12 mmol, 2.00 eq), N1-((ethylimino)methylene)—N3,N3-dimethylpropane-1,3-diamine hydrochloride (23 mg, 0.12 mmol, 2.00 eq), diisopropylethylamine (38 mg, 0.30 mmol, 0.1 mL, 5.00 eq) and (2S,4R)-1-[(2S)-2-amino-3,3-dimethyl-butanoyl]-N-[(1S)-1-[4-(2,6-difluorophenyl)phenyl]ethyl]-4-hydroxy-pyrrolidine-2-carboxamide (40 mg, 0.08 mmol, 1.36 eq, hydrochloride) at 20° C. and the mixture was stirred at 20° C. for 2 h. The mixture was diluted with water (20 mL) and extracted with ethyl acetate (20 mL×3). The combined organic layers were washed with water (20 mL), brine (20 mL), dried over sodium sulfate and then concentrated under reduced pressure to give a residue. The residue was purified by preparative thin layer chromatography (dichloromethane: methanol=10:1, R_{f}=0.3) to afford the desired product (20 mg, 0.02 mmol, 26% yield) as a light yellow solid. LCMS (ESI, m/z): 1288.5 [M+H]⁺.

Step 8: (2S,4R)-1-((2S)-2-(3-(3-((2S)-2-((((7S)-7-(2-amino-3-cyano-7-fluorobenzo[b]thiophen-4-yl)-4-(3,8-diazabicyclo[3.2.1]octan-3-yl)-8-fluoro-6-(trifluoromethyl)quinazolin-2-yl)oxy)methyl)pyrrolidin-1-yl)propoxy)propanamido)-3,3-dimethylbutanoyl)-N—((S)-1-(2′,6′-difluoro-[1,1′-biphenyl]-4-yl)ethyl)-4-hydroxypyrrolidine-2-carboxamide

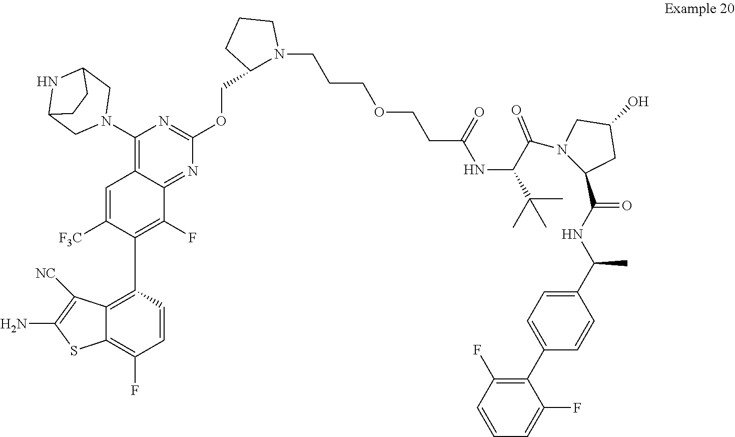

To a solution of tert-butyl 3-((S)-7-(2-amino-3-cyano-7-fluorobenzo[b]thiophen-4-yl)-2-(2-((S)-1-(3-(3-(((S)-1-((2S,4R)-2-(((S)-1-(2′,6′-difluoro-[1,1′-biphenyl]-4-yl)ethyl)carbamoyl)-4-hydroxypyrrolidin-1-yl)-3,3-dimethyl-1-oxobutan-2-yl)amino)-3-oxopropoxy)propyl)pyrrolidin-2-yl)ethyl)-8-fluoro-6-(trifluoromethyl)quinazolin-4-yl)-3,8-diazabicyclo[3.2.1]octane-8-carboxylate (25 mg, 0.02 mmol, 1.00 eq) in dichloromethane (5 mL) was added trifluoroacetic acid (1.54 g, 13.51 mmol, 1.0 mL, 695.50 eq) at 20° C. The mixture was stirred at 20° C. for 0.5 h. The mixture was concentrated under reduced pressure to give a residue. The residue was purified by preparative high performance liquid chromatography (column: Unisil 3-100 C18 Ultra 150*50 mm*3 um; mobile phase: [water(FA)-ACN]; B %: 20%-50%, 7 min) to give the desired product (15.97 mg, 0.01 mmol, 66% yield, 98% purity, formate[1]) as a yellow solid. LCMS (ESI, m/z): 1188.0 [M+H]⁺. ¹H NMR (400 MHz, DMSO) δ 8.40 (d, J=7.6 Hz, 1H), 8.19 (s, 1H), 8.09 (d, J=7.2 Hz, 3H), 7.84 (d, J=9.2 Hz, 1H), 7.53-7.37 (m, 5H), 7.28-7.18 (m, 3H), 7.14 (t, J=8.8 Hz, 1H), 4.98-4.89 (m, 1H), 4.52 (d, J=9.2 Hz, 1H), 4.48-4.31 (m, 5H), 4.28 (s, 1H), 4.11 (dd, J=7.6, 10.4 Hz, 1H), 3.76 (s, 3H), 3.63-3.55 (m, 3H), 3.41-3.35 (m, 2H), 3.10-3.02 (m, 1H), 2.96-2.80 (m, 2H), 2.42-2.11 (m, 5H), 2.07-1.98 (m, 1H), 1.97-1.87 (m, 1H), 1.85-1.62 (m, 11H), 1.39 (d, J=7.2 Hz, 3H), 0.90 (s, 9H).

Example 21: (2S,4R)-1-((2S)-2-(3-(3-((2S)-2-(((7-(2-amino-3-cyano-7-fluorobenzo[b]thiophen-4-yl)-4-(3,8-diazabicyclo[3.2.1]octan-3-yl)-6-(trifluoromethyl)quinazolin-2-yl)oxy)methyl)pyrrolidin-1-yl)propoxy)propanamido)-3,3-dimethylbutanoyl)-N—((S)-1-(2′,6′-difluoro-[1,1′-biphenyl]-4-yl)ethyl)-4-hydroxypyrrolidine-2-carboxamide

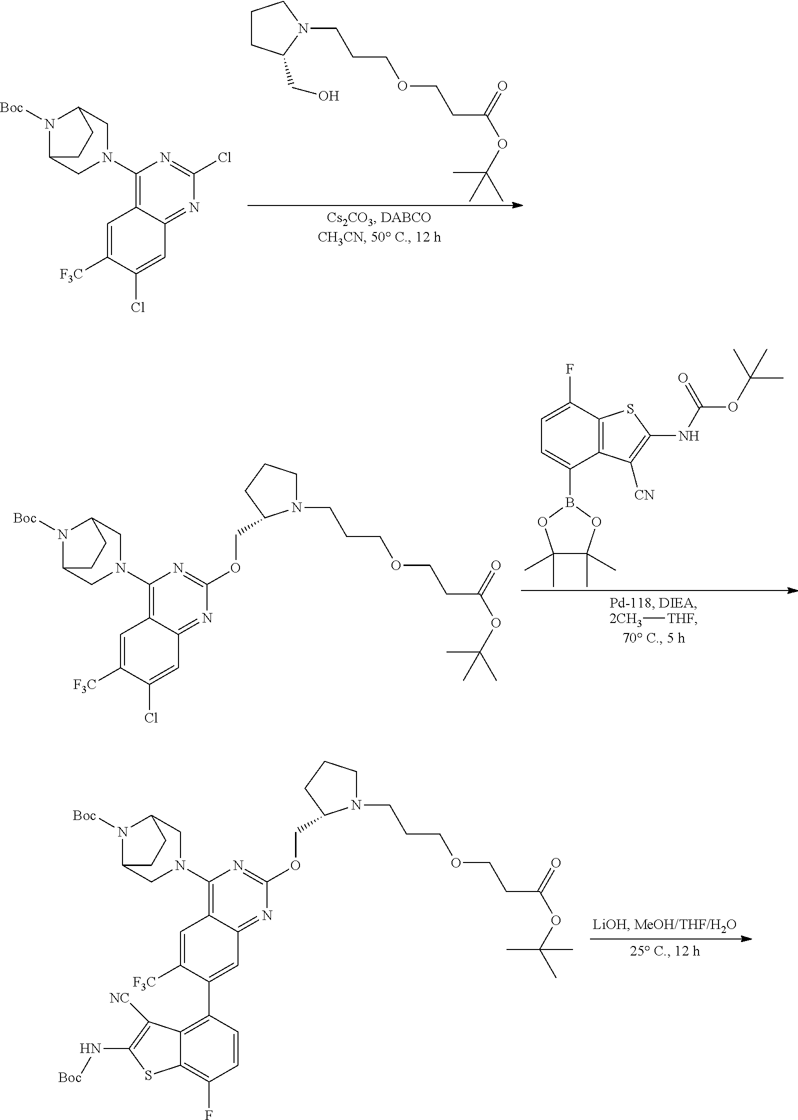

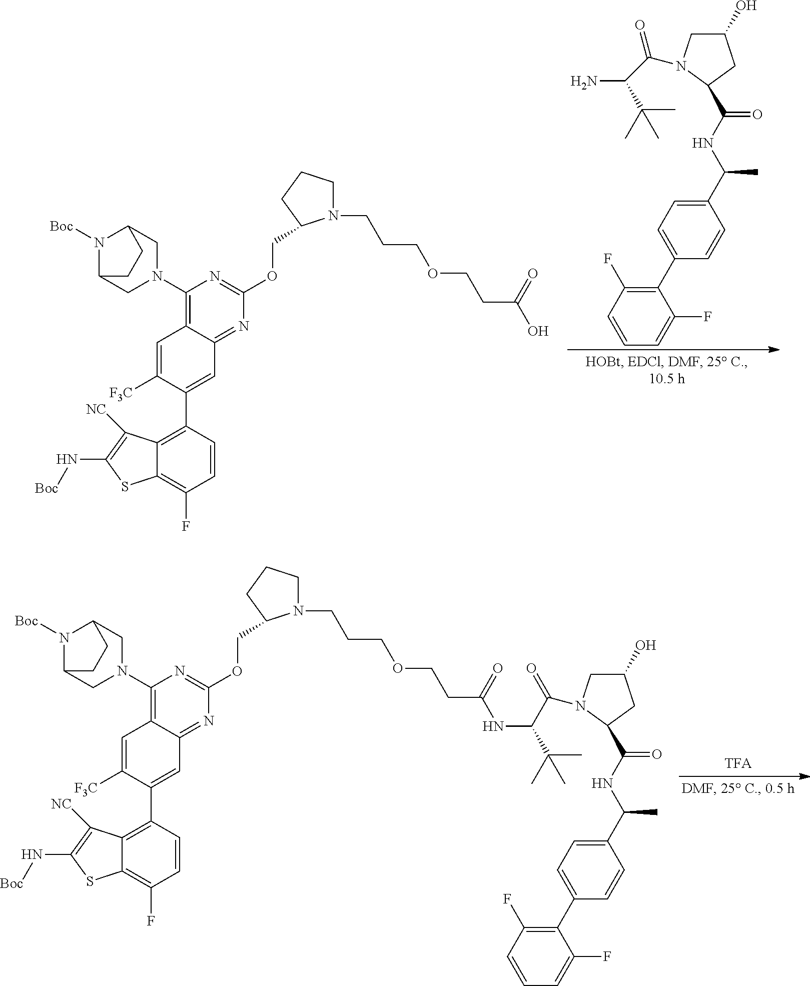 [SMILES: CC(C)(C)OC(=O)Nc1sc2c(F)ccc(-c3cc4nc(OC[C@@H]5CCCN5CCCOCCC(=O)O)nc(N5CC6CCC(C5)N6C(=O)OC(C)(C)C)c4cc3C(F)(F)F)c2c1C#N ; C[C@H](NC(=O)[C@@H]1C[C@@H](O)CN1C(=O)[C@@H](N)C(C)(C)C)c1ccc(-c2c(F)cccc2F)cc1 ; C[C@H](NC(=O)[C@@H]1C[C@@H](O)CN1C(=O)[C@@H](NC(=O)CCOCCCN1CCC[C@H]1COc1nc(N2CC3CCC(C2)N3C(=O)OC(C)(C)C)c2cc(C(F)(F)F)c(-c3ccc(F)c4sc(NC(=O)OC(C)(C)C)c(C#N)c34)cc2n1)C(C)(C)C)c1ccc(-c2c(F)cccc2F)cc1]

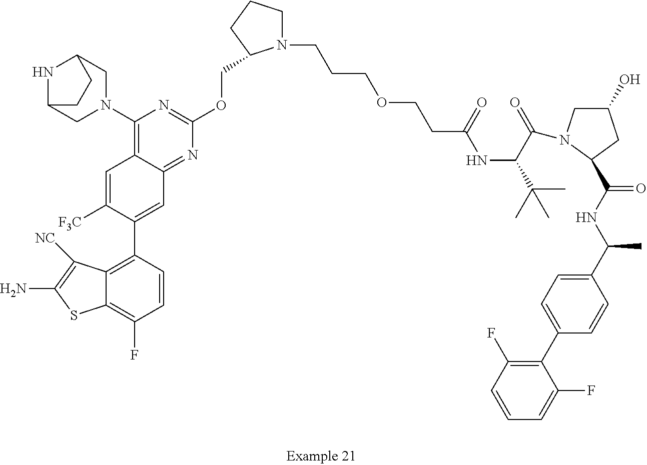 [SMILES: C[C@H](NC(=O)[C@@H]1C[C@@H](O)CN1C(=O)[C@@H](NC(=O)CCOCCCN1CCC[C@H]1COc1nc(N2CC3CCC(C2)N3)c2cc(C(F)(F)F)c(-c3ccc(F)c4sc(N)c(C#N)c34)cc2n1)C(C)(C)C)c1ccc(-c2c(F)cccc2F)cc1]

Step 1: tert-butyl 3-(2-(((S)-1-(3-(3-(tert-butoxy)-3-oxopropoxy)propyl)pyrrolidin-2-yl)methoxy)-7-chloro-6-(trifluoromethyl)quinazolin-4-yl)-3,8-diazabicyclo[3.2.1]octane-8-carboxylate

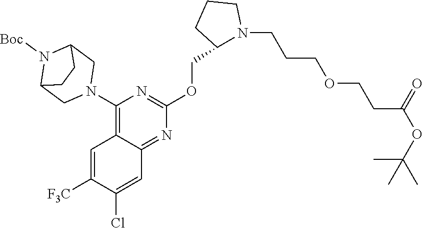 [SMILES: CC(C)(C)OC(=O)CCOCCCN1CCC[C@H]1COc1nc(N2CC3CCC(C2)N3C(=O)OC(C)(C)C)c2cc(C(F)(F)F)c(Cl)cc2n1]

To a solution of tert-butyl 3-[2,7-dichloro-6-(trifluoromethyl)quinazolin-4-yl]-3,8-diazabicyclo[3.2.1]octane-8-carboxylate (500 mg, 1.05 mmol, 1.00 eq) and tert-butyl 3-[3-[(2S)-2-(hydroxymethyl)pyrrolidin-1-yl]propoxy]propanoate (602 mg, 2.10 mmol, 2.00 eq) in acetonitrile (10 mL), was added cesium carbonate (682 mg, 2.10 mmol, 2.00 eq) and 1,4-diazabicyclo[2.2.2]octane (11 mg, 0.10 mmol, 0.10 eq). The mixture was stirred at 50° C. for 12 h. LCMS showed that the desired mass was detected. The mixture was filtered, and the filtrate was collected and concentrated to get the residue. The residue was purified by prep-TLC (9% methanol in dichloromethane) to afford the product (200 mg, 0.27 mmol, 26.2% yield) as a yellow solid. LCMS (ESI) m/z: 728.1 [M+H]⁺, ¹HNMR (400 MHz, DMSO-d₆) δ: (s, 1H), 7.78 (s, 1H), 4.42-4.38 (m, 3H), 4.26-4.23 (m, 2H), 4.10-4.07 (m, 1H), 3.59-3.51 (m, 4H), 3.39 (t, J=6.4 Hz, 2H), 3.07-3.02 (m, 1H), 2.95-2.90 (m, 1H), 2.75-2.73 (m, 1H), 2.41-2.30 (m, 2H), 2.23-2.10 (m, 1H), 1.97-1.86 (m, 1H), 1.83-1.70 (m, 3H), 1.72-1.57 (m, 7H), 1.46 (s, 9H), 1.35 (s, 9H).

Step 2: tert-butyl 3-(2-(((S)-1-(3-(3-(tert-butoxy)-3-oxopropoxy)propyl)pyrrolidin-2-yl)methoxy)-7-(2-((tert-butoxycarbonyl)amino)-3-cyano-7-fluorobenzo[b]thiophen-4-yl)-6-(trifluoromethyl)quinazolin-4-yl)-3,8-diazabicyclo[3.2.1]octane-8-carboxylate

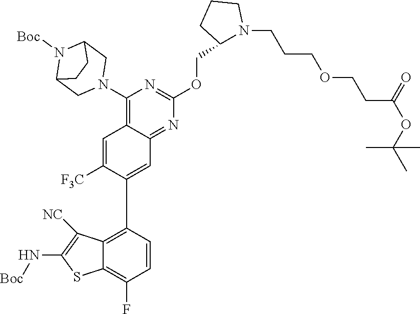 [SMILES: CC(C)(C)OC(=O)CCOCCCN1CCC[C@H]1COc1nc(N2CC3CCC(C2)N3C(=O)OC(C)(C)C)c2cc(C(F)(F)F)c(-c3ccc(F)c4sc(NC(=O)OC(C)(C)C)c(C#N)c34)cc2n1]

To a solution of tert-butyl 3-(2-(((S)-1-(3-(3-(tert-butoxy)-3-oxopropoxy)propyl)pyrrolidin-2-yl)methoxy)-7-chloro-6-(trifluoromethyl)quinazolin-4-yl)-3,8-diazabicyclo[3.2.1]octane-8-carboxylate (600 mg, 0.82 mmol, 1.00 eq) and tert-butyl N-[3-cyano-7-fluoro-4-(4,4,5,5-tetramethyl-1,3,2-dioxaborolan-2-yl)benzothiophen-2-yl]carbamate (413 mg, 0.99 mmol, 1.20 eq) in 2-methyltetrahydrofuran (12 mL) and water (3 mL), was added N,N-diisopropylethylamine (319 mg, 2.47 mmol, 3.00 eq) and di-tert-butyl(cyclopentyl)phosphane; dichloropalladium; iron (53 mg, 0.08 mmol, 0.10 eq). The mixture was stirred at 70° C. for 5 h under nitrogen. LCMS showed that the desired mass was detected. Ethyl acetate (20 mL) and water (30 mL) were added before the mixture was extracted with ethyl acetate (20 mL×3). The combined organic layers were washed with brine (20 mL), dried over anhydrous sodium sulfate, filtered and concentrated under reduced pressure to get the residue. The residue was purified by silica gel column chromatography (dichloromethane:methanol=40:1 to 10:1) to afford the crude product. The crude product was purified by preparative high performance liquid chromatography (column: Phenomenex luna C18 150*40 mm*15 um; mobile phase:[water(FA)-ACN]; B %: 45%-75%, 10 min) to afford the product (180 mg, 0.18 mmol, 22.2% yield) as a brown solid. LCMS (ESI) m/z: 984.1 [M+H]⁺

Step 3: 3-(3-((2S)-2-(((4-(8-(tert-butoxycarbonyl)-3,8-diazabicyclo[3.2.1]octan-3-yl)-7-(2-((tert-butoxycarbonyl)amino)-3-cyano-7-fluorobenzo[b]thiophen-4-yl)-6-(trifluoromethyl)quinazolin-2-yl)oxy)methyl)pyrrolidin-1-yl)propoxy)propanoic acid

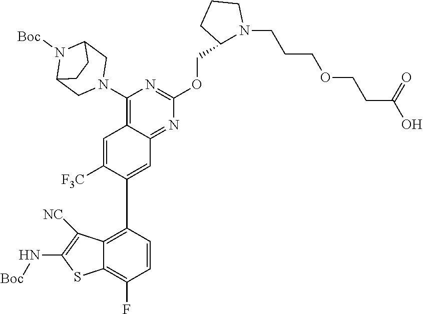 [SMILES: CC(C)(C)OC(=O)Nc1sc2c(F)ccc(-c3cc4nc(OC[C@@H]5CCCN5CCCOCCC(=O)O)nc(N5CC6CCC(C5)N6C(=O)OC(C)(C)C)c4cc3C(F)(F)F)c2c1C#N]

To a solution of tert-butyl 3-(2-(((S)-1-(3-(3-(tert-butoxy)-3-oxopropoxy)propyl)pyrrolidin-2-yl)methoxy)-7-(2-((tert-butoxycarbonyl)amino)-3-cyano-7-fluorobenzo[b]thiophen-4-yl)-6-(trifluoromethyl)quinazolin-4-yl)-3,8-diazabicyclo[3.2.1]octane-8-carboxylate (150 mg, 0.15 mmol, 1.00 eq) in the mixture solvent of tetrahydrofuran (2.5 mL), methanol (2.5 mL) and water (2.5 mL) was added lithium hydroxide (64 mg, 1.52 mmol, 10.00 eq). The mixture was stirred at 25° C. for 12 h. LCMS showed that the desired mass was detected. The mixture was concentrated under reduced pressure to remove the solvent, and hydrogen chloride (1M) was used to adjust the pH=5. Then the mixture was extracted with ethyl acetate (20 mL×3), the combined organic layers were dried over anhydrous sodium sulfate, filtered and concentrated under reduced pressure to get the residue. The residue was purified by prep-TLC (9% methanol in dichloromethane) to afford the product (110 mg, 0.12 mmol, 77.7% yield) as a yellow solid. LCMS (ESI) m/z: 928.1 [M+H]⁺.

Step 4: tert-butyl 3-(7-(2-((tert-butoxycarbonyl)amino)-3-cyano-7-fluorobenzo[b]thiophen-4-yl)-2-(((S)-1-(3-(3-(((S)-1-((2S,4R)-2-(((S)-1-(2′,6′-difluoro-[1,1′-biphenyl]-4-yl)ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-6-(trifluoromethyl)quinazolin-4-yl)-3,8-diazabicyclo[3.2.1]octane-8-carboxylate

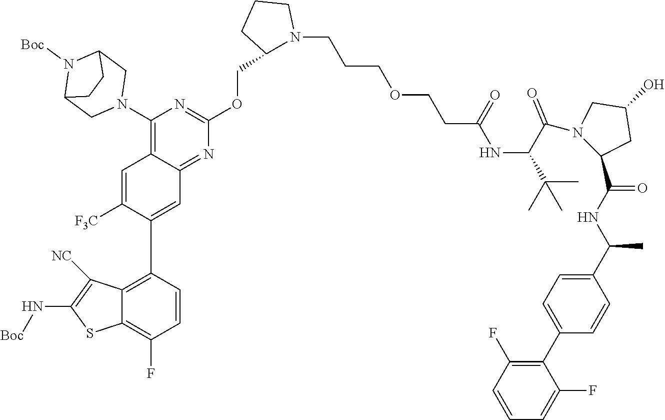 [SMILES: C[C@H](NC(=O)[C@@H]1C[C@@H](O)CN1C(=O)[C@@H](NC(=O)CCOCCCN1CCC[C@H]1COc1nc(N2CC3CCC(C2)N3C(=O)OC(C)(C)C)c2cc(C(F)(F)F)c(-c3ccc(F)c4sc(NC(=O)OC(C)(C)C)c(C#N)c34)cc2n1)C(C)(C)C)c1ccc(-c2c(F)cccc2F)cc1]

To a solution of 3-(3-((2S)-2-(((4-(8-(tert-butoxycarbonyl)-3,8-diazabicyclo[3.2.1]octan-3-yl)-7-(2-((tert-butoxycarbonyl)amino)-3-cyano-7-fluorobenzo[b]thiophen-4-yl)-6-(trifluoromethyl)quinazolin-2-yl)oxy)methyl)pyrrolidin-1-yl)propoxy)propanoic acid (55 mg, 0.06 mmol, 1.00 eq) and (2S,4R)-1-[(2S)-2-amino-3,3-dimethyl-butanoyl]-N-[(1S)-1-[4-(2,6-difluorophenyl)phenyl]ethyl]-4-hydroxy-pyrrolidine-2-carboxamide (51 mg, 0.09 mmol, 1.50 eq, trifluoroacetic acid) in N,N-dimethylformamide (2 mL), was added N,N-diisopropylethylamine (38 mg, 0.30 mmol, 5.00 eq) and 1-hydroxybenzotriazole (16 mg, 0.12 mmol, 2.00 eq). The mixture was stirred at 25° C. for 0.5 h. Then, N-(3-dimethylaminopropyl)-N-ethylcarbodiimide hydrochloride (23 mg, 0.12 mmol, 2.00 eq) was added and the mixture was stirred at 25° C. for 10 h. LCMS showed that the desired mass was detected. Ethyl acetate (20 mL) and water (20 mL) were added before the mixture extracted with ethyl acetate (20 mL×3). The combined organic layers were washed with brine (20 mL×3), dried over anhydrous sodium sulfate, filtered and concentrated under reduced pressure to get the residue. The residue was purified by prep-TLC (9% methanol in dichloromethane) to afford the product (60 mg, 0.04 mmol, 73.9% yield) as a yellow solid. LCMS (ESI) m/z: 1369.3 [M+H]⁺.

Step 5: (2S,4R)-1-((2S)-2-(3-(3-((2S)-2-(((7-(2-amino-3-cyano-7-fluorobenzo [b]thiophen-4-yl)-4-(3,8-diazabicyclo[3.2.1]octan-3-yl)-6-(trifluoromethyl)quinazolin-2-yl)oxy)methyl)pyrrolidin-1-yl)propoxy)propanamido)-3,3-dimethylbutanoyl)-N—((S)-1-(2′,6′-difluoro-[1,1′-biphenyl]-4-yl)ethyl)-4-hydroxypyrrolidine-2-carboxamide (Example 21)

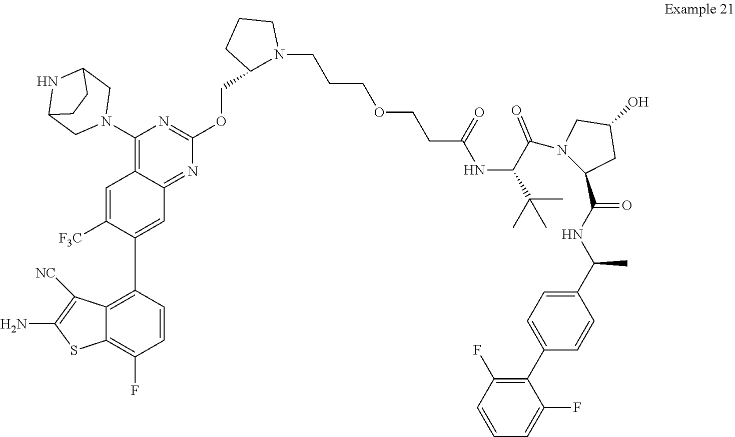 [SMILES: C[C@H](NC(=O)[C@@H]1C[C@@H](O)CN1C(=O)[C@@H](NC(=O)CCOCCCN1CCC[C@H]1COc1nc(N2CC3CCC(C2)N3)c2cc(C(F)(F)F)c(-c3ccc(F)c4sc(N)c(C#N)c34)cc2n1)C(C)(C)C)c1ccc(-c2c(F)cccc2F)cc1]

To a solution of tert-butyl 3-(7-(2-((tert-butoxycarbonyl)amino)-3-cyano-7-fluorobenzo[b]thiophen-4-yl)-2-(((S)-1-(3-(3-(((S)-1-((2S,4R)-2-(((S)-1-(2′,6′-difluoro-[1,1′-biphenyl]-4-yl)ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-6-(trifluoromethyl)quinazolin-4-yl)-3,8-diazabicyclo[3.2.1]octane-8-carboxylate (60 mg, 0.04 mmol, 1.00 eq) in dichloromethane (3 mL), was added trifluoroacetic acid (13.51 mmol, 1.0 mL, 308.28 eq). The mixture was stirred at 25° C. for 0.5 h. LCMS showed that the desired mass was detected. The reaction mixture was concentrated under reduced pressure to afford the residue. The residue was purified by preparative high performance liquid chromatography (column: Unisil 3-100 C18 Ultra 150*50 mm*3 um; mobile phase: [water(FA)-ACN]; B %: 19%-49%, 7 min) to afford the desired product (27.89 mg, 0.022 mmol, 51.0% yield, 97.4% purity, formate[1]) as a yellow solid. LCMS (ESI) m/z: 1169.30 [M+H]⁺, ¹HNMR (400 MHz, DMSO-d₆) δ: 8.39 (d, J=8.4 Hz, 1H), 8.21 (s, 1H), 8.16 (s, 1H), 7.98 (s, 1H), 7.87-7.78 (m, 1H), 7.51-7.43 (m, 2H), 7.39 (s, 4H), 7.24-7.17 (m, 3H), 7.08 (t, J=8.8 Hz, 1H), 4.96-4.88 (m, 1H), 4.51 (d, J=9.2 Hz, 1H), 4.45-4.26 (m, 5H), 4.13-4.05 (m, 1H), 3.82 (s, 2H), 3.72-3.65 (m, 2H), 3.63-3.42 (m, 8H), 3.07-3.02 (m, 1H), 2.95-2.82 (m, 2H), 2.46-2.36 (m, 2H), 2.28-2.19 (m, 2H), 2.04-1.97 (m, 1H), 1.95-1.89 (m, 1H), 1.80-1.61 (m, 10H), 1.38 (d, J=6.8 Hz, 3H), 0.89 (s, 9H).

Example 22: (2S,4R)-1-((2S)-2-(3-(3-((2S)-2-(((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)azetidin-1-yl)propoxy)propanamido)-3,3-dimethylbutanoyl)-4-hydroxy-N—((S)-1-(4-(4-methylthiazol-5-yl)phenyl)ethyl)pyrrolidine-2-carboxamide

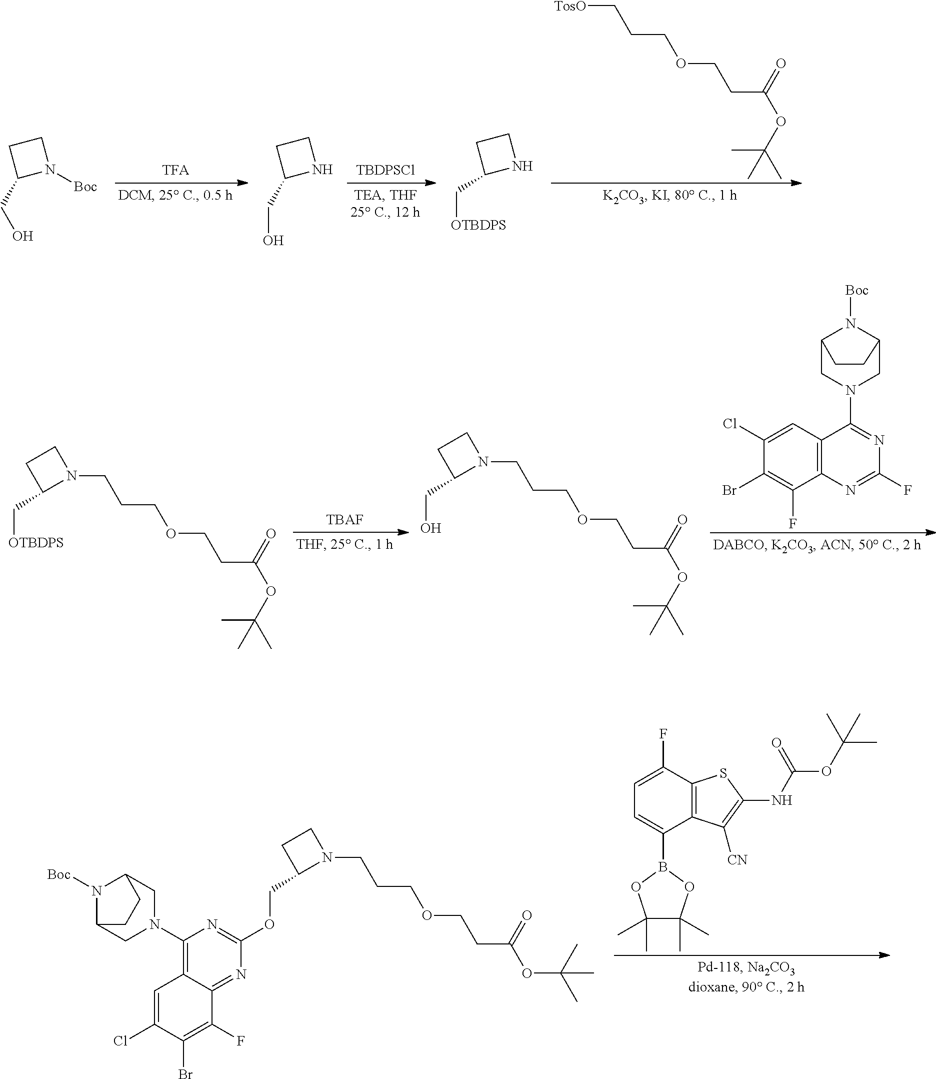

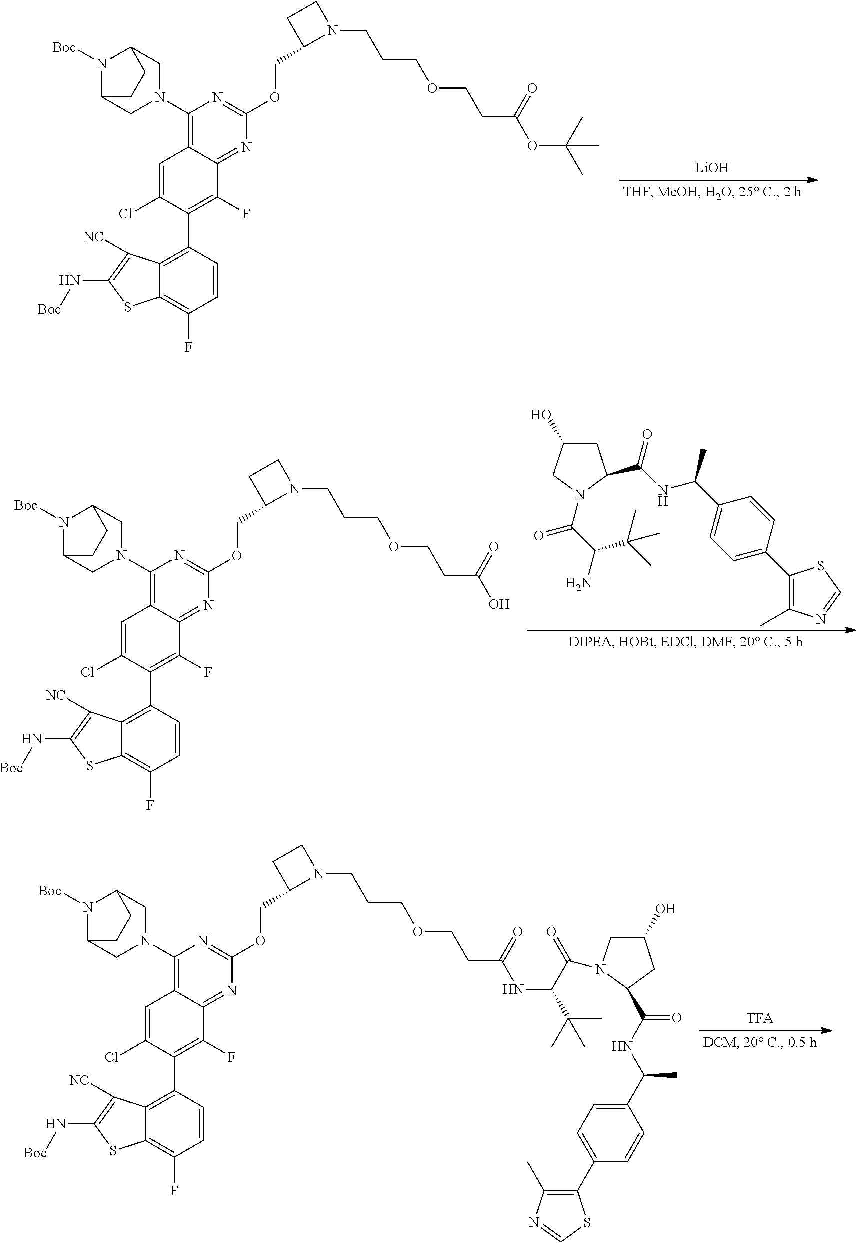

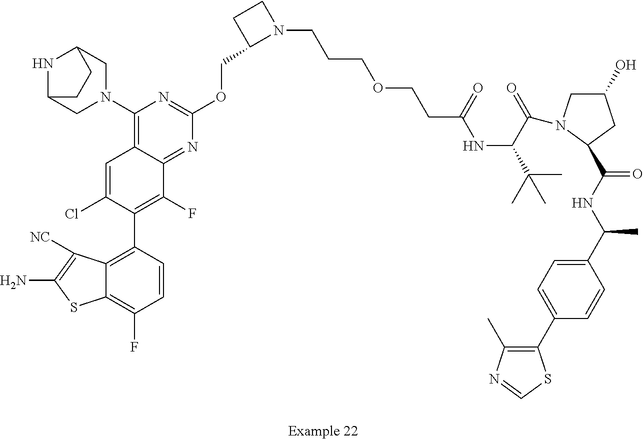 [SMILES: Cc1ncsc1-c1ccc([C@H](C)NC(=O)[C@@H]2C[C@@H](O)CN2C(=O)[C@@H](NC(=O)CCOCCCN2CC[C@H]2COc2nc(N3CC4CCC(C3)N4)c3cc(Cl)c(-c4ccc(F)c5sc(N)c(C#N)c45)c(F)c3n2)C(C)(C)C)cc1]

Step 1: (S)-azetidin-2-ylmethanol

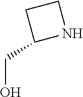

To a solution of tert-butyl (S)-2-(hydroxymethyl)azetidine-1-carboxylate (13 g, 69.43 mmol) in dichloromethane (60 mL) was added trifluoroacetic acid (38.50 g, 337.65 mmol). The mixture was stirred at 25° C. for 0.5 h. The reaction mixture was concentrated under reduced pressure to afford the crude product [(2S)-azetidin-2-yl]methanol (13.00 g, 64.63 mmol, 93% yield, trifluoroacetate) as a yellow oil, which was used into next step directly.

Step 2: (S)-2-(((tert-butyldiphenylsilyl)oxy)methyl)azetidine

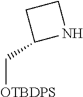

A mixture of (S)-azetidin-2-ylmethanol (13.00 g, 64.63 mmol, trifluoroacetate), tert-butyldiphenyl chlorosilane (23.10 g, 84.02 mmol), triethylamine (19.62 g, 193.89 mmol) and 4-dimethylaminopyridine (790 mg, 6.46 mmol) in tetrahydrofuran (200 mL) was stirred at 25° C. for 12 h. The reaction mixture was concentrated under reduced pressure to give a residue. The residue was purified by preparative high performance liquid chromatography (column: Phenomenex luna C18 (250*70 mm, 10 um); mobile phase: [water(FA)-ACN]; B %: 25%-55%, 21 min) to afford the product (7.50 g, 20.74 mmol, 32% yield) as a yellow oil. LCMS (ESI, m/z): 326.4 [M+H]⁺.

Step 3: tert-butyl (S)-3-(3-(2-(((tert-butyldiphenylsilyl)oxy)methyl)azetidin-1-yl)propoxy)propanoate

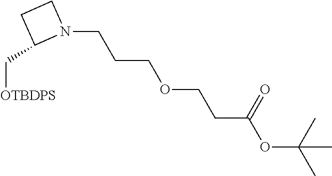

A mixture of (S)-2-(((tert-butyldiphenylsilyl)oxy)methyl)azetidine (2.00 g, 6.14 mmol), tert-butyl 3-[3-(p-tolylsulfonyloxy)propoxy]propanoate (1.32 g, 3.69 mmol), potassium carbonate (1.02 g, 7.37 mmol), potassium iodide (61 mg, 0.37 mmol) in dimethylformamide (30 mL) was stirred at 80° C. for 1 h. The reaction mixture was quenched by water (150 mL) at 25° C., then the mixture was extracted with ethyl acetate (100 mL×3). The combined organic layers were washed with brine (50 mL), dried over sodium sulfate, filtered and the filtrate was concentrated under reduced pressure to give a residue. The residue was purified by silica gel column chromatography (0-100% ethyl acetate in petroleum ether) to afford the product (0.60 g, 1.17 mmol, 32% yield) as a yellow oil. LCMS (ESI, m/z): 512.4 [M+H]⁺.

Step 4: tert-butyl (S)-3-(3-(2-(hydroxymethyl)azetidin-1-yl)propoxy)propanoate

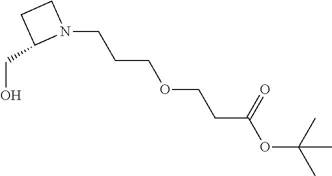 [SMILES: CC(C)(C)OC(=O)CCOCCCN1CC[C@H]1CO]

To a solution of tert-butyl (S)-3-(3-(2-(((tert-butyldiphenylsilyl)oxy)methyl)azetidin-1-yl)propoxy)propanoate (1.50 g, 2.93 mmol) in tetrahydrofuran (5 mL) was added tetrabutylammonium fluoride (1 M, 5.9 mL). The mixture was stirred at 25° C. for 1 h. The reaction mixture was concentrated under reduced pressure to get a residue. The residue was purified by silica gel column chromatography (eluent: PE/EtOAc=0/1) to afford tert-butyl (S)-3-(3-(2-(hydroxymethyl)azetidin-1-yl)propoxy)propanoate (800 mg, 2.93 mmol, 99% yield) as a yellow oil. ¹H NMR (400 MHz, CHLOROFORM-d) (4.11 (q, J=7.2 Hz, 1H), 3.63 (t, J=6.4 Hz, 2H), 3.44 (t, J=6.4 Hz, 2H), 3.39-3.22 (m, 3H), 2.85-2.77 (m, 1H), 2.62 (td, J=7.6, 11.6 Hz, 1H), 2.49-2.44 (m, 2H), 2.42-2.38 (m, 1H), 2.22-2.09 (m, 1H), 2.06-1.99 (m, 1H), 1.95-1.85 (m, 1H), 1.65-1.55 (m, 2H), 1.47-1.42 (m, 9H)

Step 5: tert-butyl 3-(7-bromo-2-(((S)-1-(3-(3-(tert-butoxy)-3-oxopropoxy)propyl)azetidin-2-yl)methoxy)-6-chloro-8-fluoroquinazolin-4-yl)-3,8-diazabicyclo [3.2.1]octane-8-carboxylate

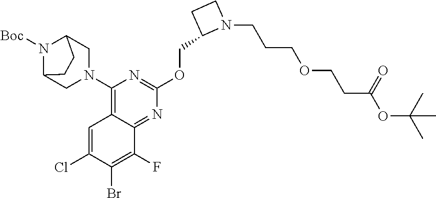 [SMILES: CC(C)(C)OC(=O)CCOCCCN1CC[C@H]1COc1nc(N2CC3CCC(C2)N3C(=O)OC(C)(C)C)c2cc(Cl)c(Br)c(F)c2n1]

A mixture of tert-butyl (S)-3-(3-(2-(hydroxymethyl)azetidin-1-yl)propoxy)propanoate (400 mg, 1.46 mmol), tert-butyl 3-(7-bromo-6-chloro-2,8-difluoro-quinazolin-4-yl)-3,8-diazabicyclo[3.2.1]octane-8-carboxylate (477 mg, 0.98 mmol), 1,4-diazabicyclo[2.2.2]octane (11 mg, 0.10 mmol), cesium carbonate (954 mg, 2.93 mmol) in acetonitrile (8 mL) was stirred at 50° C. for 2 h. The resulting product was dissolved in ethyl acetate, filtered, and the filtrate was evaporated under reduced pressure to get a residue. The residue was purified by prep-TLC (9% methanol in dichloromethane) to afford the product (70 mg, 0.10 mmol, 10% yield) as a yellow oil. LCMS (ESI, m/z): 743.10, 744.2 [M+H]⁺.

Step 6: tert-butyl 3-(2-(((S)-1-(3-(3-(tert-butoxy)-3-oxopropoxy)propyl)azetidin-2-yl)methoxy)-7-(2-((tert-butoxycarbonyl)amino)-3-cyano-7-fluorobenzo[b]thiophen-4-yl)-6-chloro-8-fluoroquinazolin-4-yl)-3,8-diazabicyclo[3.2.1]octane-8-carboxylate

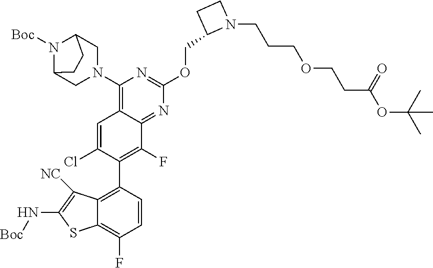 [SMILES: CC(C)(C)OC(=O)CCOCCCN1CC[C@H]1COc1nc(N2CC3CCC(C2)N3C(=O)OC(C)(C)C)c2cc(Cl)c(-c3ccc(F)c4sc(NC(=O)OC(C)(C)C)c(C#N)c34)c(F)c2n1]

A mixture of tert-butyl 3-(7-bromo-2-(((S)-1-(3-(3-(tert-butoxy)-3-oxopropoxy)propyl)azetidin-2-yl)methoxy)-6-chloro-8-fluoroquinazolin-4-yl)-3,8-diazabicyclo[3.2.1]octane-8-carboxylate (140 mg, 0.20 mmol), tert-butyl N-[3-cyano-7-fluoro-4-(4,4,5,5-tetramethyl-1,3,2-dioxaborolan-2-yl)benzothiophen-2-yl]carbamate (158 mg, 0.38 mmol), sodium carbonate (40 mg, 0.38 mmol), ditert-butyl(cyclopentyl)phosphane; dichloropalladium; iron (12 mg, 0.02 mmol) in dioxane (12 mL) and water (3 mL) was degassed and purged with nitrogen for 3 times, the mixture was stirred at 90° C. for 2 h under nitrogen atmosphere. The reaction mixture was quenched by the addition of water (20 mL) at 25° C., extracted with ethyl acetate (20 mL×3). The combined organic layers were washed with brine (10 mL), dried over sodium sulfate, filtered and the filtrate was concentrated under reduced pressure to give a residue. The residue was purified by prep-TLC (9% methanol in dichloromethane) to afford the product (25 mg, 0.03 mmol, 14% yield) as a yellow solid. LCMS (ESI, m/z): 954.0 [M+H]⁺.

Step 7: 3-(3-((2S)-2-(((4-(8-(tert-butoxycarbonyl)-3,8-diazabicyclo[3.2.1]octan-3-yl)-7-(2-((tert-butoxycarbonyl)amino)-3-cyano-7-fluorobenzo[b]thiophen-4-yl)-6-chloro-8-fluoroquinazolin-2-yl)oxy)methyl)azetidin-1-yl)propoxy)propanoic acid

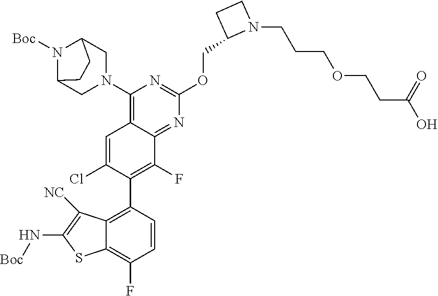 [SMILES: CC(C)(C)OC(=O)Nc1sc2c(F)ccc(-c3c(Cl)cc4c(N5CC6CCC(C5)N6C(=O)OC(C)(C)C)nc(OC[C@@H]5CCN5CCCOCCC(=O)O)nc4c3F)c2c1C#N]

To a solution of tert-butyl 3-(2-(((S)-1-(3-(3-(tert-butoxy)-3-oxopropoxy)propyl)azetidin-2-yl)methoxy)-7-(2-((tert-butoxycarbonyl)amino)-3-cyano-7-fluorobenzo[b]thiophen-4-yl)-6-chloro-8-fluoroquinazolin-4-yl)-3,8-diazabicyclo[3.2.1]octane-8-carboxylate (25 mg, 0.03 mmol) in tetrahydrofuran (0.5 mL), methanol (0.5 mL) and water (0.3 mL) was added lithium hydroxide (13 mg, 0.52 mmol). The mixture was stirred at 25° C. for 2 h. The reaction mixture was concentrated under reduced pressure to give a residue. The residue was diluted with water (5 mL), and hydrochloric acid (1M) was added to adjust the pH=4-6. Then the mixture was extracted with ethyl acetate (10 mL×2), the combined organic layers were washed with brine (10 mL), dried over sodium sulfate, filtered and the filtrate was concentrated under reduced pressure to afford the product (20 mg, 0.02 mmol, 85% yield) as a yellow solid. LCMS (ESI, m/z): 898.3 [M]⁺.

Step 8: tert-butyl 3-(7-(2-((tert-butoxycarbonyl)amino)-3-cyano-7-fluorobenzo[b]thiophen-4-yl)-6-chloro-8-fluoro-2-(((S)-1-(3-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropoxy)propyl)azetidin-2-yl)methoxy)quinazolin-4-yl)-3,8-diazabicyclo[3.2.1]octane-8-carboxylate

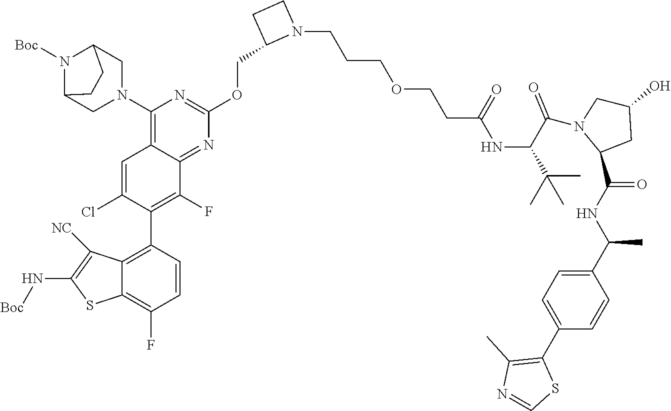 [SMILES: Cc1ncsc1-c1ccc([C@H](C)NC(=O)[C@@H]2C[C@@H](O)CN2C(=O)[C@@H](NC(=O)CCOCCCN2CC[C@H]2COc2nc(N3CC4CCC(C3)N4C(=O)OC(C)(C)C)c3cc(Cl)c(-c4ccc(F)c5sc(NC(=O)OC(C)(C)C)c(C#N)c45)c(F)c3n2)C(C)(C)C)cc1]

To a solution of 3-(3-((2S)-2-(((4-(8-(tert-butoxycarbonyl)-3,8-diazabicyclo[3.2.1]octan-3-yl)-7-(2-((tert-butoxycarbonyl)amino)-3-cyano-7-fluorobenzo[b]thiophen-4-yl)-6-chloro-8-fluoroquinazolin-2-yl)oxy)methyl)azetidin-1-yl)propoxy)propanoic acid (20 mg, 0.02 mmol), (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (12 mg, 0.02 mmol, hydrochloride) in dimethylformamide (2 mL) was added diisopropylethylamine (14 mg, 0.10 mmol), 1-hydroxybenzotriazole (9 mg, 0.07 mmol), 1-(3-dimethylaminopropyl)-3-ethylcarbodiimide hydrochloride (13 mg, 0.07 mmol). The mixture was stirred at 25° C. for 5 h. The reaction mixture was quenched by water (10 mL) at 25° C., extracted with ethyl acetate (20 mL×3). The combined organic layers were washed with brine (10 mL), dried over sodium sulfate, filtered and the filtrate was concentrated under reduced pressure to give a residue. The residue was purified by prep-TLC (9% methanol in dichloromethane) to afford the product (10 mg, 0.01 mmol, 34% yield) as a yellow oil. LCMS (ESI, m/z): 1323.5 [M+H]⁺.

Step 9: (2S,4R)-1-((2S)-2-(3-(3-((2S)-2-(((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)azetidin-1-yl)propoxy)propanamido)-3,3-dimethylbutanoyl)-4-hydroxy-N—((S)-1-(4-(4-methylthiazol-5-yl)phenyl)ethyl)pyrrolidine-2-carboxamide (Example 22)

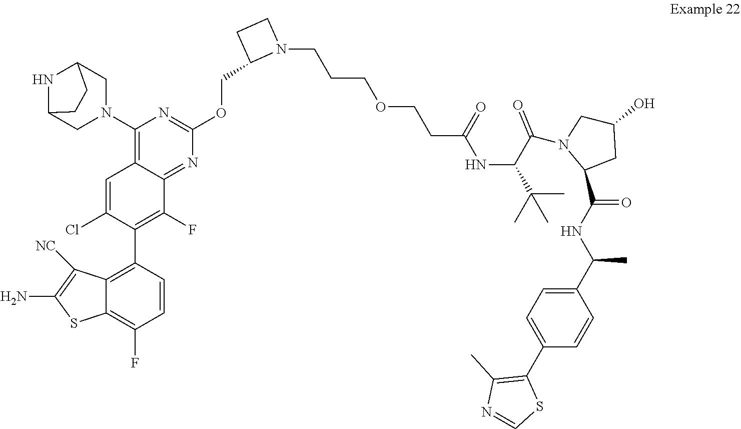 [SMILES: Cc1ncsc1-c1ccc([C@H](C)NC(=O)[C@@H]2C[C@@H](O)CN2C(=O)[C@@H](NC(=O)CCOCCCN2CC[C@H]2COc2nc(N3CC4CCC(C3)N4)c3cc(Cl)c(-c4ccc(F)c5sc(N)c(C#N)c45)c(F)c3n2)C(C)(C)C)cc1]

To a solution of tert-butyl 3-(7-(2-((tert-butoxycarbonyl)amino)-3-cyano-7-fluorobenzo[b]thiophen-4-yl)-6-chloro-8-fluoro-2-(((S)-1-(3-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropoxy)propyl)azetidin-2-yl)methoxy)quinazolin-4-yl)-3,8-diazabicyclo[3.2.1]octane-8-carboxylate (10 mg, 0.01 mmol) in dichloromethane (1 mL) was added trifluoroacetic acid (1.54 g, 13.51 mmol). The mixture was stirred at 25° C. for 15 min. The reaction mixture was concentrated under reduced pressure to give a residue. The residue was purified by preparative high performance liquid chromatography (column: Phenomenex C18 75*30 mm*3 um; mobile phase: [water(FA)-ACN]; B %: 15%-45%, 7 min) to afford the desired product (2.7 mg, 0.01 mmol, 30% yield, 97% purity, formate[1]) as a white solid. LCMS (ESI, m/z): 1124.5 [M+H]⁺. ¹H NMR (400 MHz, DMSO) δ 8.99 (s, 1H), 8.38 (d, J=7.2 Hz, 1H), 8.26 (s, 2H), 8.11 (s, 2H), 7.86-7.80 (m, 2H), 7.45-7.42 (m, 2H), 7.40-7.36 (m, 2H), 7.29-7.26 (m, 1H), 7.18-7.12 (m, 1H), 5.33 (t, J=5.2 Hz, 1H), 4.95-4.87 (m, 1H), 4.52 (d, J=9.2 Hz, 1H), 4.35-4.23 (m, 7H), 3.61-3.56 (m, 5H), 2.73 (br d, J=8.0 Hz, 2H), 2.67-2.57 (m, 3H), 2.46-2.44 (m, 3H), 2.28 (br d, J=6.4 Hz, 1H), 2.01-1.97 (m, 3H), 1.94-1.90 (m, 1H), 1.83-1.75 (m, 2H), 1.64-1.61 (m, 2H), 1.54-1.49 (m, 3H), 1.37 (d, J=7.2 Hz, 3H), 1.26-1.23 (m, 5H), 0.93-0.90 (m, 9H)

Example 23: (2S,4R)-1-((2S)-2-(5-((2S)-2-(((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)azetidin-1-yl)pentanamido)-3,3-dimethylbutanoyl)-4-hydroxy-N—((S)-1-(4-(4-methylthiazol-5-yl)phenyl)ethyl)pyrrolidine-2-carboxamide

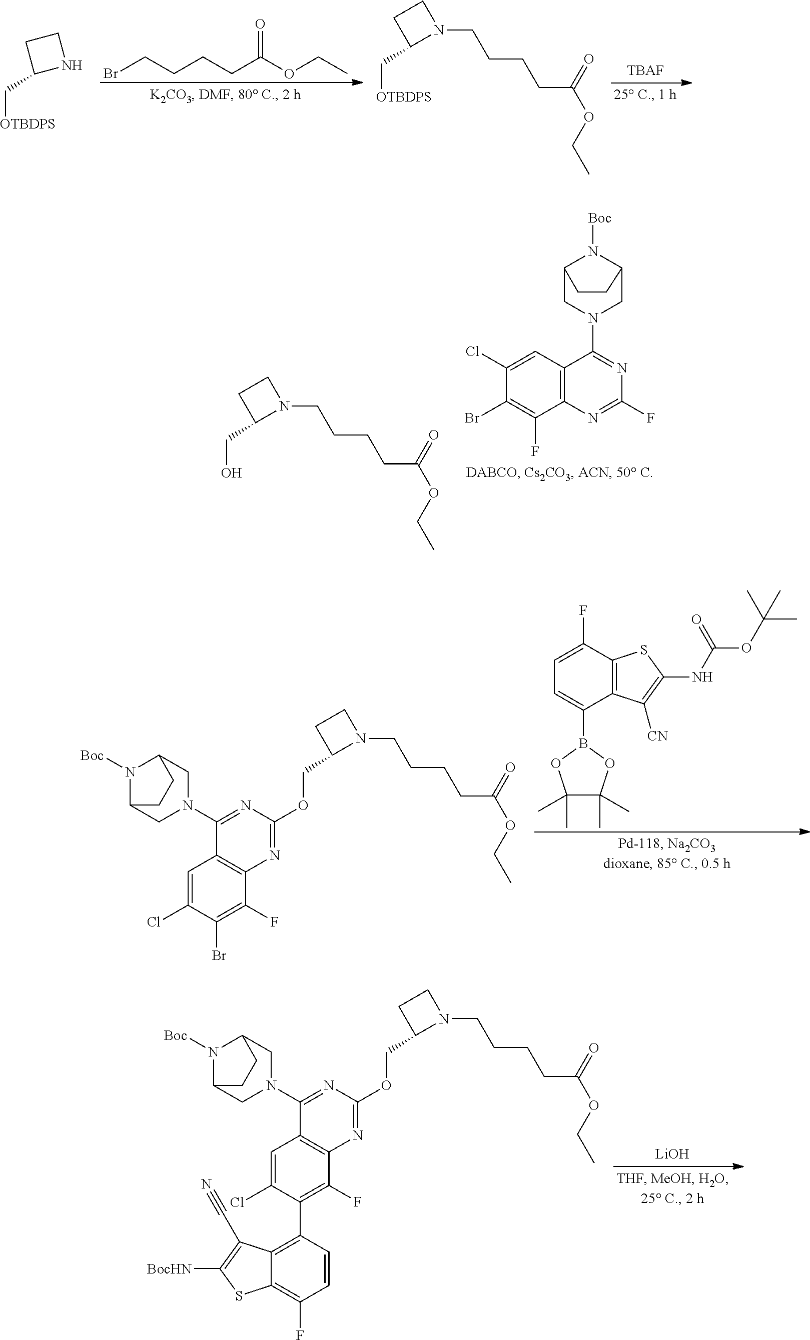

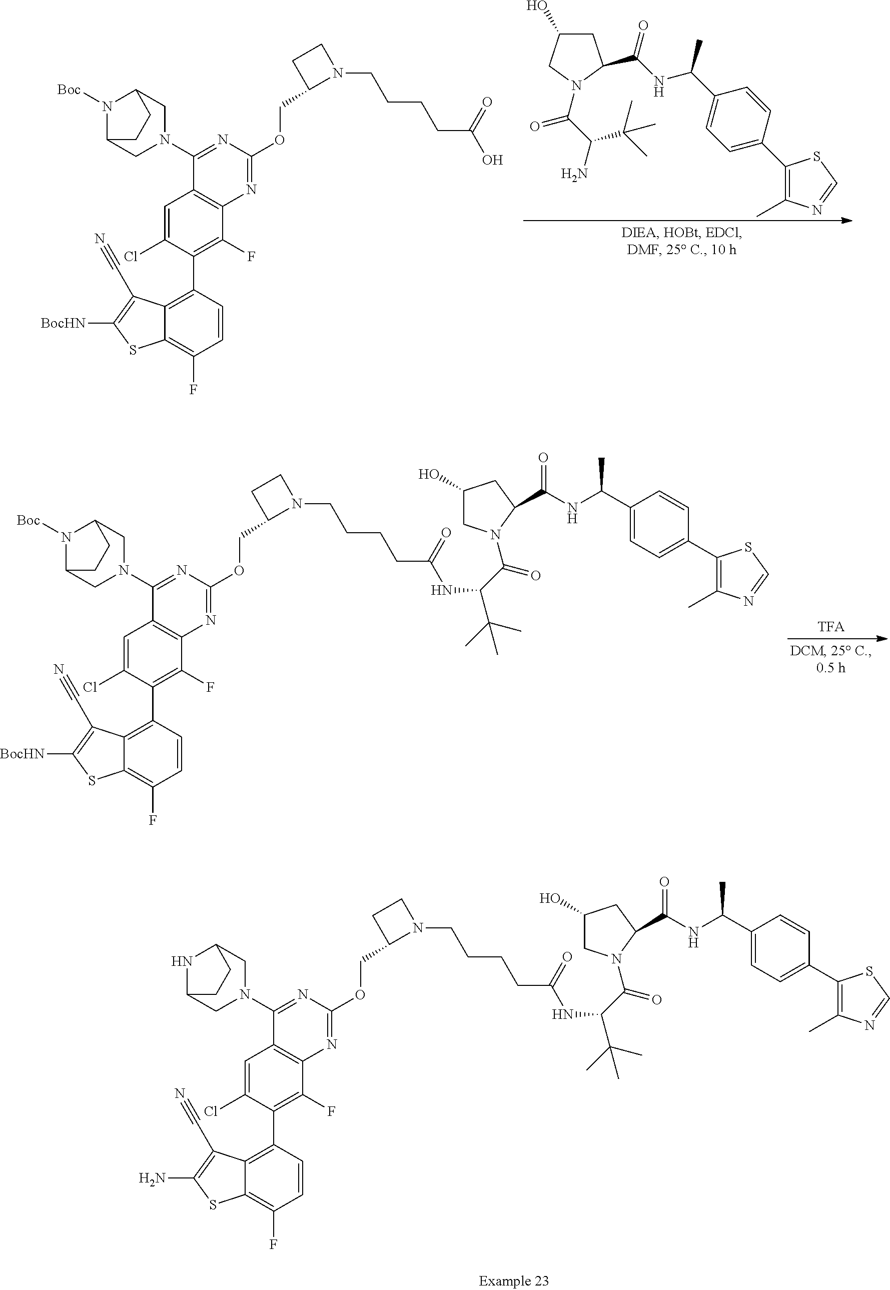

Step 1: ethyl (S)-5-(2-(((tert-butyldiphenylsilyl)oxy)methyl)azetidin-1-yl)pentanoate

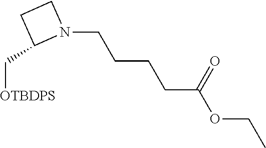

To a solution of [(2S)-azetidin-2-yl]methoxy-tert-butyl-diphenyl-silane (1.90 g, 5.84 mmol, 1.00 eq) and ethyl 5-bromopentanoate (1.34 g, 6.42 mmol, 1 mL, 1.10 eq) in N,N-dimethylformamide (15 mL) was added potassium carbonate (2.42 g, 17.51 mmol, 3.00 eq). The mixture was stirred at 80° C. for 2 h. LCMS and TLC (dichloromethane/methanol=10/1) showed the reaction was completed. The mixture was diluted with water (60 mL) and extracted with ethyl acetate (20 mL×3). The organic layer was concentrated under reduced pressure to give a residue. The residue was purified by column chromatography on silica gel (petroleum ether/ethyl acetate=10/1 to 0/1) to afford the desired product (1.30 g, 2.87 mmol, 49% yield) as a yellow oil. LCMS (ESI, m/z): 454.4 [M+H]⁺

Step 2: ethyl (S)-5-(2-(hydroxymethyl)azetidin-1-yl)pentanoate

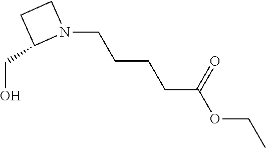

To a solution of ethyl 5-[(2S)-2-[[tert-butyl(diphenyl)silyl]oxymethyl]azetidin-1-yl]pentanoate (1.30 g, 2.87 mmol, 1.00 eq) in tetrahydrofuran (15 mL) was added tetrabutylammonium fluoride (1 M in tetrahydrofuran, 2.9 mL, 1.00 eq). The mixture was stirred at 25° C. for 1 h. TLC (dichloromethane/methanol=10/1) showed the reaction was completed. The mixture was concentrated under reduced pressure to give a residue. The residue was purified by column chromatography on silica gel (petroleum ether/ethyl acetate=10/1 to 0/1) to afford the product (370 mg, 1.72 mmol, 60% yield) as an off-white oil. ¹H NMR (400 MHz, CDCl₃) δ: 4.14 (q, J=7.2 Hz, 2H), 3.62 (dd, J=11.6, 3.2 Hz, 1H), 3.49-3.31 (m, 3H), 2.96-2.84 (m, 1H), 2.63 (dt, J=11.2, 7.6 Hz, 1H), 2.43 (dt, J=11.6, 7.2 Hz, 1H), 2.34-2.29 (m, 2H), 2.27-2.20 (m, 1H), 2.00-1.94 (m, 1H), 1.72-1.59 (m, 2H), 1.44 (q, J=7.6 Hz, 2H), 1.28 (t, J=7.2 Hz, 3H).

Step 3: tert-butyl 3-(7-bromo-6-chloro-2-(((S)-1-(5-ethoxy-5-oxopentyl)azetidin-2-yl)methoxy)-8-fluoroquinazolin-4-yl)-3,8-diazabicyclo[3.2.1]octane-8-carboxylate

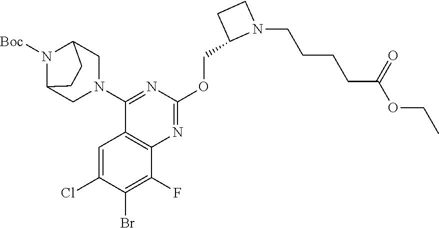 [SMILES: CCOC(=O)CCCCN1CC[C@H]1COc1nc(N2CC3CCC(C2)N3C(=O)OC(C)(C)C)c2cc(Cl)c(Br)c(F)c2n1]

To a solution of tert-butyl 3-(7-bromo-6-chloro-2,8-difluoro-quinazolin-4-yl)-3,8-diazabicyclo[3.2.1]octane-8-carboxylate (455 mg, 0.93 mmol, 1.00 eq), ethyl 5-[(2S)-2-(hydroxymethyl)azetidin-1-yl]pentanoate (200 mg, 0.93 mmol, 1.00 eq) and cesium carbonate (605 mg, 1.86 mmol, 2.00 eq) in acetonitrile (8 mL) was added 1,4-diazabicyclo[2.2.2]octane (10.42 mg, 0.09 umol, 0.1 eq). The mixture was stirred at 50° C. for 10 h. LCMS showed the reaction was completed. The mixture was concentrated under reduced pressure to give a residue. The residue was purified by preparative high layer chromatography (9% methanol in dichloromethane) to afford the product (200 mg, 0.30 mmol, 31% yield) as a yellow oil. LCMS (ESI, m/z): 684.2, 686.1 [M+H]⁺. ¹H NMR (400 MHz, CDCl₃) δ: 8.04-7.93 (m, 1H), 4.38-4.28 (m, 4H), 4.22 (br s, 2H), 4.00 (q, J=7.2 Hz, 2H), 3.56 (br dd, J=10.0, 3.6 Hz, 2H), 3.32 (br s, 1H), 3.29-3.22 (m, 1H), 3.17 (d, J=5.2 Hz, 1H), 2.63-2.56 (m, 1H), 2.32-2.25 (m, 1H), 2.22 (t, J=7.6 Hz, 2H), 2.05-1.98 (m, 1H), 1.96-1.90 (m, 1H), 1.78 (br s, 2H), 1.64 (br d, J=8.4 Hz, 2H), 1.52-1.48 (m, 1H), 1.46 (s, 9H), 1.33-1.22 (m, 3H), 1.14 (t, J=7.2 Hz, 3H).

Step 4: tert-butyl 3-(7-(2-((tert-butoxycarbonyl)amino)-3-cyano-7-fluorobenzo[b]thiophen-4-yl)-6-chloro-2-(((S)-1-(5-ethoxy-5-oxopentyl)azetidin-2-yl)methoxy)-8-fluoroquinazolin-4-yl)-3,8-diazabicyclo[3.2.1]octane-8-carboxylate

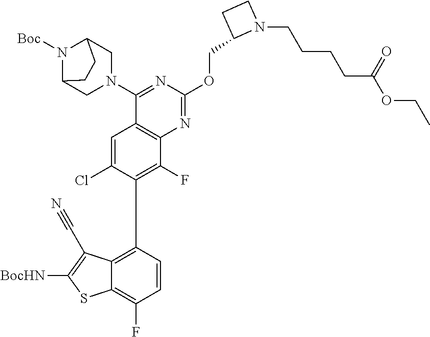 [SMILES: CCOC(=O)CCCCN1CC[C@H]1COc1nc(N2CC3CCC(C2)N3C(=O)OC(C)(C)C)c2cc(Cl)c(-c3ccc(F)c4sc(NC(=O)OC(C)(C)C)c(C#N)c34)c(F)c2n1]

To a solution of tert-butyl 3-[7-bromo-6-chloro-2-[[(2S)-1-(5-ethoxy-5-oxo-pentyl)azetidin-2-yl]methoxy]-8-fluoro-quinazolin-4-yl]-3,8-diazabicyclo[3.2.1]octane-8-carboxylate (200 mg, 030 mmol, 1.00 eq), i-butyl N-[3-cyano-7-fluoro-4-(4,4,5,5-tetramethyl-1,3,2-dioxaborolan-2-yl)benzothiophen-2-yl]carbamate (146 mg, 0.35 mmol, 1.20 eq) and sodium carbonate (62 mg, 0.60 mmol, 2.00 eq) in dioxane (3 mL) and water (0.3 mL) was added ditert-butyl(cyclopentyl)phosphane; dichloropalladium; iron (19 mg, 0.03 mmol, 0.10 eq). The mixture was degassed and purged with nitrogen for 3 times and stirred at 85° C. for 0.5 h. LCMS showed the reaction was completed. The mixture was filtered to give a residue. The residue was purified by preparative high layer chromatography (column: Phenomenex Synergi C18 150*25 mm*10 um; mobile phase: [water(FA)-ACN]; B %: 38%-68%, 10 min) to afford the product (80 mg, 0.09 mmol, 30% yield) as a yellow solid. LCMS (ESI, m/z): 896.5 [M+H]⁺.

Step 5: 5-((2S)-2-(((4-(8-(tert-butoxycarbonyl)-3,8-diazabicyclo[3.2.1]octan-3-yl)-7-(2-((tert-butoxycarbonyl)amino)-3-cyano-7-fluorobenzo[b]thiophen-4-yl)-6-chloro-8-fluoroquinazolin-2-yl)oxy)methyl)azetidin-1-yl)pentanoic acid

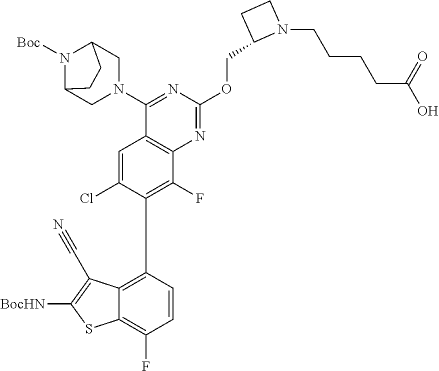 [SMILES: CC(C)(C)OC(=O)Nc1sc2c(F)ccc(-c3c(Cl)cc4c(N5CC6CCC(C5)N6C(=O)OC(C)(C)C)nc(OC[C@@H]5CCN5CCCCC(=O)O)nc4c3F)c2c1C#N]

To a solution of tert-butyl 3-[7-[2-(tert-butoxycarbonylamino)-3-cyano-7-fluoro-benzothiophen-4-yl]-6-chloro-2-[[(2S)-1-(5-ethoxy-5-oxo-pentyl)azetidin-2-yl]methoxy]-8-fluoro-quinazolin-4-yl]-3,8-diazabicyclo[3.2.1]octane-8-carboxylate (80 mg, 0.09 mmol, 1.00 eq) in methanol (3 mL), tetrahydrofuran (3 mL) and water (2 mL) was added lithium hydroxide (4 mg, 0.18 mmol, 2.00 eq). The mixture was stirred at 25° C. for 2 h. LCMS showed the reaction was completed. The mixture was concentrated under reduced pressure to remove the methanol. The residue was extracted with ethyl acetate (5 mL×2). The combined organic layers were concentrated under reduced pressure to afford the crude product (76 mg, 0.09 mmol, 98% yield) as a yellow solid, which was used into the next step without further purification. LCMS (ESI, m/z): 868.2 [M+H]⁺.

Step 6: tert-butyl 3-(7-(2-((tert-butoxycarbonyl)amino)-3-cyano-7-fluorobenzo[b]thiophen-4-yl)-6-chloro-8-fluoro-2-(((S)-1-(5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentyl)azetidin-2-yl)methoxy)quinazolin-4-yl)-3,8-diazabicyclo[3.2.1]octane-8-carboxylate

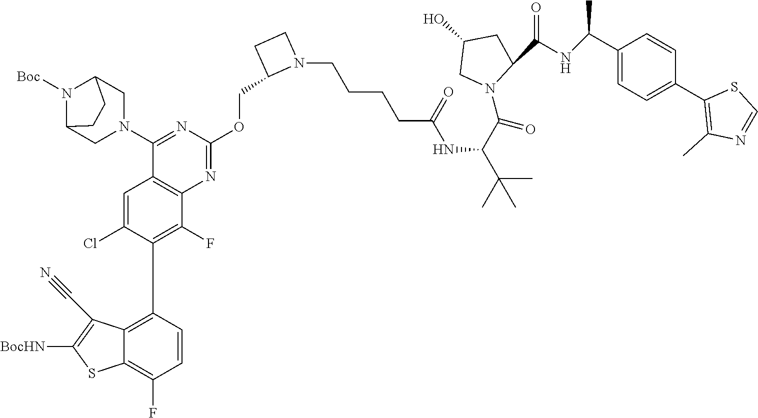 [SMILES: Cc1ncsc1-c1ccc([C@H](C)NC(=O)[C@@H]2C[C@@H](O)CN2C(=O)[C@@H](NC(=O)CCCCN2CC[C@H]2COc2nc(N3CC4CCC(C3)N4C(=O)OC(C)(C)C)c3cc(Cl)c(-c4ccc(F)c5sc(NC(=O)OC(C)(C)C)c(C#N)c45)c(F)c3n2)C(C)(C)C)cc1]

To a solution of 5-[(2S)-2-[[7-[2-(tert-butoxycarbonylamino)-3-cyano-7-fluoro-benzothiophen-4-yl]-4-(8-tert-butoxycarbonyl-3,8-diazabicyclo[3.2.1]octan-3-yl)-6-chloro-8-fluoro-quinazolin-2-yl]oxymethyl]azetidin-1-yl]pentanoic acid (40 mg, 0.05 mmol, 1.00 eq) in N,N-dimethylformamide (1 mL) was added diisopropylethylamine (30 mg, 0.02 mmol, 5.00 eq), hydroxybenzotriazole (8 mg, 0.06 mmol, 1.20 eq), 1-(3-dimethylaminopropyl)-3-ethylcarbodiimide hydrochloride (11 mg, 0.06 mmol, 1.20 eq) and 1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (24 mg, 0.05 umol, 1.10 eq, hydrochloride). The mixture was stirred at 25° C. for 10 h. LCMS showed the reaction was completed. The mixture was diluted with water (10 mL) and extracted with ethyl acetate (5 mL×2). The organic layer was concentrated under reduced pressure to give a residue. The residue was purified by preparative high layer chromatography (9% methanol in dichloromethane) to afford the product (30 mg, 0.02 mmol, 50% yield) as a white solid. LCMS (ESI, m/z): 1294.8 [M+H]⁺.

Step 7: (2S,4R)-1-((2S)-2-(5-((2S)-2-(((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)azetidin-1-yl)pentanamido)-3,3-dimethylbutanoyl)-4-hydroxy-N—((S)-1-(4-(4-methylthiazol-5-yl)phenyl)ethyl)pyrrolidine-2-carboxamide (Example 23)

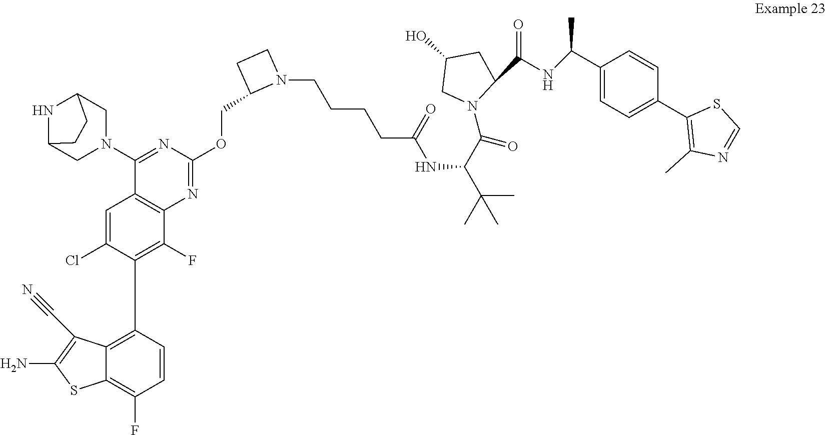 [SMILES: Cc1ncsc1-c1ccc([C@H](C)NC(=O)[C@@H]2C[C@@H](O)CN2C(=O)[C@@H](NC(=O)CCCCN2CC[C@H]2COc2nc(N3CC4CCC(C3)N4)c3cc(Cl)c(-c4ccc(F)c5sc(N)c(C#N)c45)c(F)c3n2)C(C)(C)C)cc1]

To a solution of tert-butyl 3-[7-[2-(tert-butoxycarbonylamino)-3-cyano-7-fluoro-benzothiophen-4-yl]-6-chloro-8-fluoro-2-[[(2S)-1-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-5-oxo-pentyl]azetidin-2-yl]methoxy]quinazolin-4-yl]-3,8-diazabicyclo[3.2.1]octane-8-carboxylate (30 mg, 0.02 mmol, 1.00 eq) in dichloromethane (2 mL) was added trifluoroacetic acid (2 mL). The mixture was stirred at 25° C. for 0.5 h. LCMS showed the reaction was completed. The mixture was concentrated under reduced pressure to give a residue. The residue was purified by preparative high layer chromatography (column: Phenomenex Synergi C18 150*25 mm*10 um; mobile phase: [water(FA)-ACN]; B %: 16%-36%, 10 min) to afford the desired product (12.4 mg, 0.01 mmol, 44% yield, 94% purity, formate[1]) as a white solid. LCMS (ESI, m/z): 1095.3 [M+H]⁺. ¹H NMR (400 MHz, DMSO) δ 8.99 (s, 1H), 8.37 (d, J=7.6 Hz, 1H), 8.30 (s, 1H), 8.11 (s, 2H), 7.85 (s, 1H), 7.76 (d, J=9.2 Hz, 1H), 7.47-7.42 (m, 2H), 7.41-7.33 (m, 2H), 7.27 (dd, J=8.0, 5.2 Hz, 1H), 7.20-7.12 (m, 1H), 4.95-4.88 (m, 1H), 4.51 (d, J=9.2 Hz, 1H), 4.42 (t, J=8.2 Hz, 1H), 4.34-4.22 (m, 5H), 3.60 (br s, 3H), 3.52 (br s, 4H), 3.27 (br s, 2H), 2.75-2.68 (m, 1H), 2.65-2.54 (m, 1H), 2.53 (br s, 1H), 2.46 (s, 3H), 2.31-2.18 (m, 2H), 2.15-2.07 (m, 1H), 2.05-1.97 (m, 2H), 1.96-1.88 (m, 1H), 1.84-1.73 (m, 1H), 1.69-1.55 (m, 4H), 1.52-1.41 (m, 2H), 1.37 (d, J=7.2 Hz, 3H), 1.31-1.22 (m, 2H), 0.92 (s, 9H).

Example 24: (S)-1-((2S,4R)-4-hydroxy-2-(((S)-1-(2′,3′,6′-trifluoro-[1,1′-biphenyl]-4-yl)ethyl)carbamoyl) pyrrolidin-1-yl)-3,3-dimethyl-1-oxobutan-2-yl 5-((2S)-2-(((4-(3,8-diazabicyclo[3.2.1]octan-3-yl)-7-(8-ethynyl-7-fluoro-3-((tetrahydro-2H-pyran-2-yl)oxy)naphthalen-1-yl)-8-fluoropyrido[4,3-d]pyrimidin-2-yl)oxy)methyl)pyrrolidin-1-yl)pentanoate

Intermediate 1

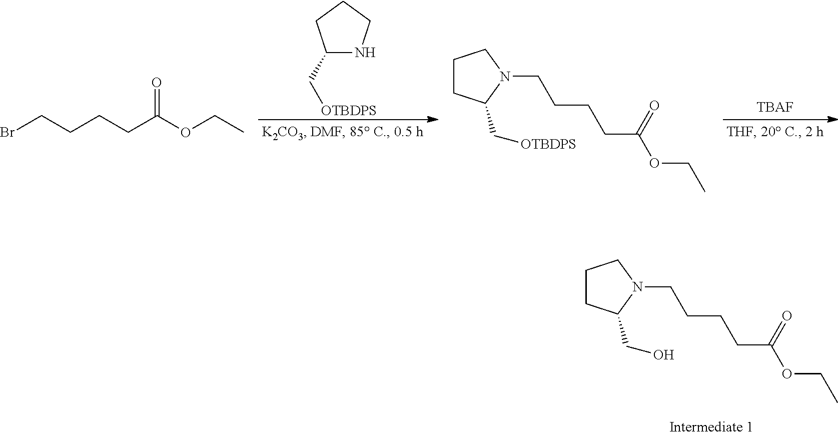

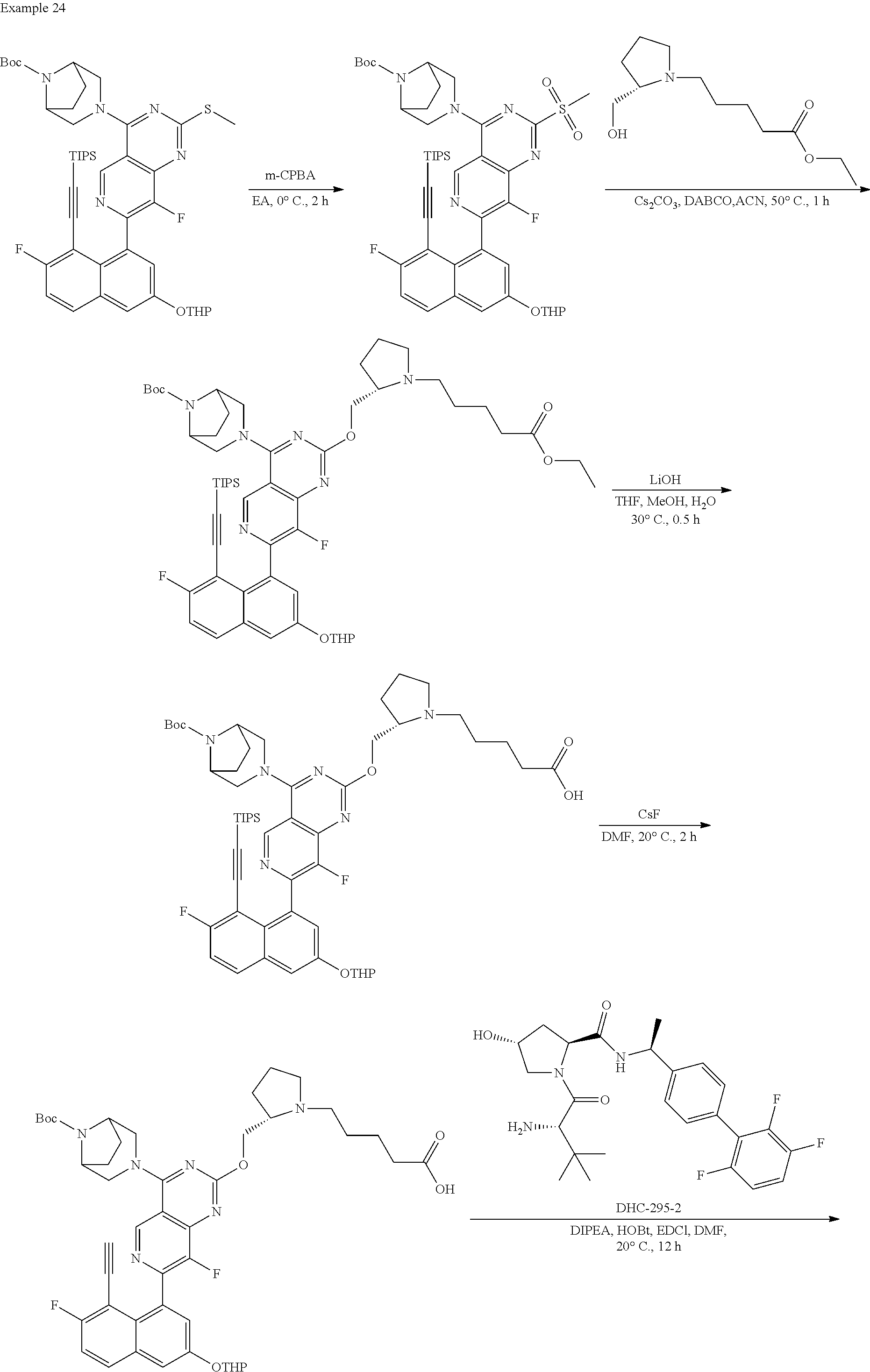

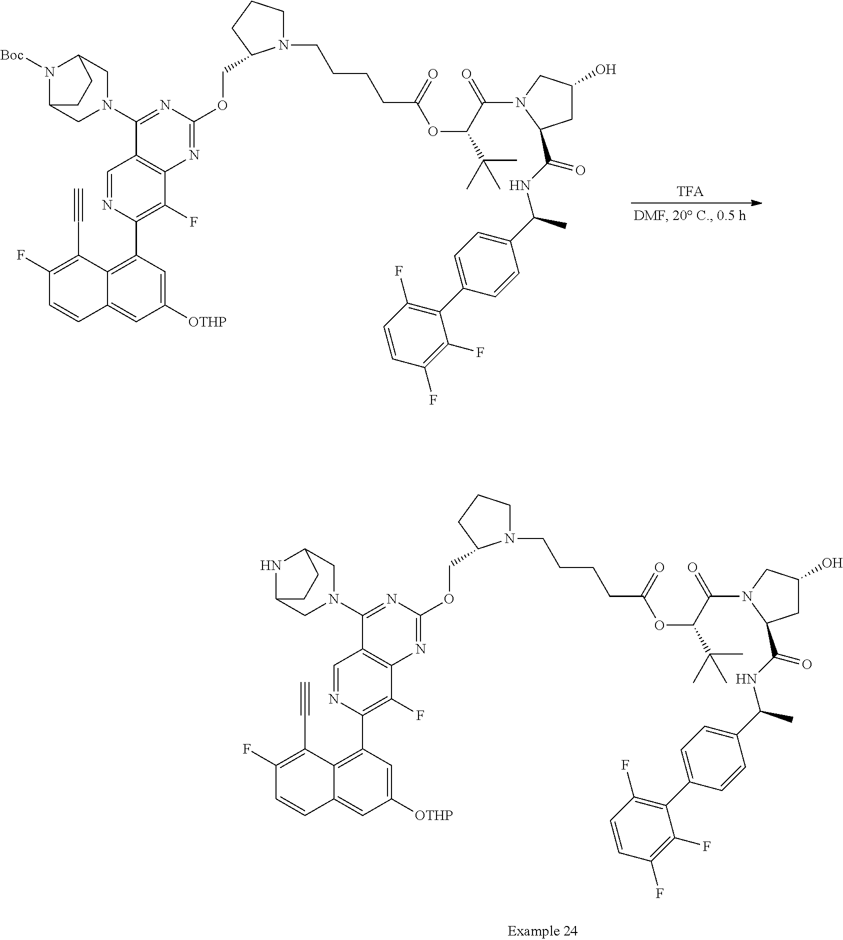

Step 1: ethyl (S)-5-(2-(((tert-butyldiphenylsilyl)oxy)methyl)pyrrolidin-1-yl)pentanoate

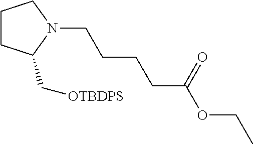

To a solution of (S)-2-(((tert-butyldiphenylsilyl)oxy)methyl)pyrrolidine (27.07 g, 79.71 mmol, 1 eq) and ethyl 5-bromopentanoate (20 g, 95.66 mmol, 1.2 eq) in N,N-dimethylformamide (200 mL) was added and potassium carbonate (27.54 g, 200 mmol, 2.5 eq). The mixture was stirred at 85° C. for 0.5 h. LCMS showed the desired mass was detected. The reaction mixture was partitioned between water (300 mL) and ethyl acetate (500 mL). The organic phase was separated, washed with brine (100 mL), dried over anhydrous sodium sulfate, filtered and concentrated under reduced pressure to give a residue. The residue was purified by silica gel chromatography (petroleum ether/ethyl acetate=1/0 to 1/1) to afford ethyl (S)-5-(2-(((tert-butyldiphenylsilyl)oxy)methyl)pyrrolidin-1-yl)pentanoate (33 g, 70 mmol, 88% yield) as a colorless oil. LCMS (ESI, m/z): 468.4 [M+H]⁺. ¹H NMR (400 MHz, CHLOROFORM) δ 7.65-7.54 (m, 4H), 7.39-7.23 (m, 6H), 4.04-3.97 (m, 2H), 3.59 (d, J=5.2, 10.0 Hz, 1H), 3.39 (d, J=7.2, 10.0 Hz, 1H), 3.07-2.87 (m, 1H), 2.73-2.59 (m, 1H), 2.52-2.41 (m, 1H), 2.24 (t, J=7.2 Hz, 1H), 2.18-2.13 (m, 2H), 2.06 (q, J=8.4 Hz, 1H), 1.82-1.77 (m, 1H), 1.75-1.66 (m, 1H), 1.62-1.55 (m, 2H), 1.48 (d, J=8.0, 15.6 Hz, 2H), 1.39-1.30 (m, 2H), 1.14 (t, J=7.2 Hz, 3H), 0.97 (s, 9H).

Step 2: ethyl (S)-5-(2-(hydroxymethyl)pyrrolidin-1-yl)pentanoate

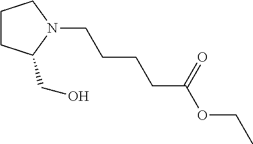 [SMILES: CCOC(=O)CCCCN1CCC[C@H]1CO]

To a solution of ethyl (S)-5-(2-(((tert-butyldiphenylsilyl)oxy)methyl)pyrrolidin-1-yl)pentanoate (13 g, 27.79 mmol, 1 eq) in tetrahydrofuran (50 mL) was added tetrabutylammonium fluoride (1 M, 56 mL, 2 eq). The mixture was stirred at 20° C. for 2 h. TLC (dichloromethane/methanol=10/1) showed the starting material was consumed completely and new spots was detected. The reaction mixture was filtered and concentrated under reduced pressure to give a residue. The residue was purified by silica gel chromatography (petroleum ether/ethyl acetate=1/0 to 0/1) to afford ethyl (S)-5-(2-(hydroxymethyl)pyrrolidin-1-yl)pentanoate (5.2 g, 22.68 mmol, 81% yield) as a yellow oil. ¹H NMR (400 MHz, CHLOROFORM) δ 4.05 (q, J=7.2 Hz, 2H), 3.51 (d, J=4.0, 10.4 Hz, 1H), 3.32 (d, J=2.4, 10.4 Hz, 1H), 3.08 (d, J=4.4, 8.8 Hz, 2H), 2.66 (d, J=8.0, 12.0 Hz, 1H), 2.52-2.42 (m, 1H), 2.27-2.22 (m, 2H), 2.21-2.08 (m, 2H), 1.86-1.74 (m, 1H), 1.72-1.60 (m, 4H), 1.55 (d, J=7.2, 14.4 Hz, 1H), 1.51-1.40 (m, 2H), 1.18 (t, J=7.2 Hz, 3H).

Step 3: tert-butyl 3-(8-fluoro-7-(7-fluoro-3-((tetrahydro-2H-pyran-2-yl)oxy)-8-((triisopropylsilyl) ethynyl)naphthalen-1-yl)-2-(methylsulfonyl)pyrido[4,3-d]pyrimidin-4-yl)-3,8-diazabicyclo[3.2.1] octane-8-carboxylate

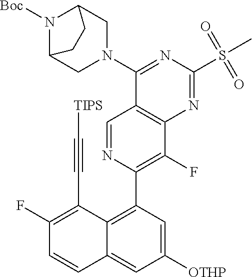

To a solution of tert-butyl 3-(8-fluoro-7-(7-fluoro-3-((tetrahydro-2H-pyran-2-yl)oxy)-8-((triisopropylsilyl)ethynyl)naphthalen-1-yl)-2-(methylthio)pyrido[4,3-d]pyrimidin-4-yl)-3,8-diazabicyclo[3.2.1]octane-8-carboxylate (2.8 g, 3.37 mmol, 1.0 eq) in ethyl acetate (30 mL) was added metachloroperbenzoic acid (2.18 g, 10.12 mmol, 80% purity, 3 eq) at 0° C., the mixture was stirred at 0° C. for 2 h. TLC (petroleum ether/ethyl acetate=2/1) showed the starting material was consumed completely and a new spot was detected. The mixture was quenched with saturated aqueous sodium sulfite (200 mL), extracted with ethyl acetate (200 mL×2), washed with brine (200 mL), dried over sodium sulfate, filtered and then concentrated under vacuum to get a residue. The residue was purified by column chromatography (silica dioxide, petroleum ether/ethyl acetate=10/1 to 1/1) to afford tert-butyl 3-(8-fluoro-7-(7-fluoro-3-((tetrahydro-2H-pyran-2-yl)oxy)-8-((triisopropylsilyl)ethynyl)naphthalen-1-yl)-2-(methylsulfonyl)pyrido[4,3-d]pyrimidin-4-yl)-3,8-diazabicyclo[3.2.1]octane-8-carboxylate (2 g, 2.32 mmol, 69% yield) as a yellow solid.

Step 4: tert-butyl 3-(2-(((S)-1-(5-ethoxy-5-oxopentyl)pyrrolidin-2-yl)methoxy)-8-fluoro-7-(7-fluoro-3-((tetrahydro-2H-pyran-2-yl)oxy)-8-((triisopropylsilyl)ethynyl)naphthalen-1-yl)pyrido[4,3-d]pyrimidin-4-yl)-3,8-diazabicyclo[3.2.1]octane-8-carboxylate

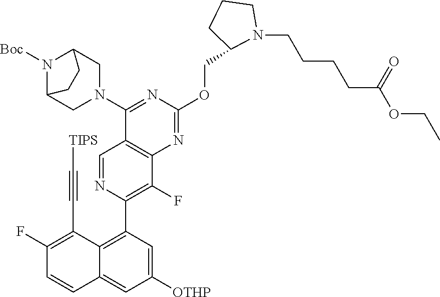

To a solution of tert-butyl 3-(8-fluoro-7-(7-fluoro-3-((tetrahydro-2H-pyran-2-yl)oxy)-8-((triisopropylsilyl) ethynyl)naphthalen-1-yl)-2-(methylsulfonyl)pyrido[4,3-d]pyrimidin-4-yl)-3,8-diazabicyclo[3.2.1] octane-8-carboxylate (300 mg, 0.35 mmol, 1 eq) in acetonitrile (5 mL) was added ethyl (S)-5-(2-(hydroxymethyl)pyrrolidin-1-yl)pentanoate (120 mg, 0.52 mmol, 1.50 eq), 1,4-diazabicyclo[2.2.2]octane (4 mg, 0.03 mmol, 0.1 eq) and cesium carbonate (340 mg, 1.04 mmol, 3 eq). The mixture was stirred at 50° C. for 1 h. TLC (dichloromethane/methanol=20/1) showed the starting material was consumed completely and a new spot was detected. The reaction mixture was filtered and concentrated under reduced pressure to give a residue. The reaction mixture was partitioned between water (20 mL) and ethyl acetate (20 mL×3). The organic phase was separated, washed with brine (20 mL), dried over anhydrous sodium sulfate, filtered and concentrated under reduced pressure to give a residue. The residue was purified by prep-TLC (dichloromethane/methanol=15/1) to afford the product tert-butyl 3-(2-(((S)-1-(5-ethoxy-5-oxopentyl)pyrrolidin-2-yl)methoxy)-8-fluoro-7-(7-fluoro-3-((tetrahydro-2H-pyran-2-yl)oxy)-8-((triisopropylsilyl)ethynyl)naphthalen-1-yl)pyrido[4,3-d]pyrimidin-4-yl)-3,8-diazabicyclo[3.2.1]octane-8-carboxylate (200 mg, 0.20 mmol, 57% yield) as a yellow oil.

Step 5: 5-((2S)-2-(((4-(8-(tert-butoxycarbonyl)-3,8-diazabicyclo[3.2.1]octan-3-yl)-8-fluoro-7-(7-fluoro-3-((tetrahydro-2H-pyran-2-yl)oxy)-8-((triisopropylsilyl)ethynyl)naphthalen-1-yl)pyrido[4,3-d]pyrimidin-2-yl)oxy)methyl)pyrrolidin-1-yl)pentanoic acid

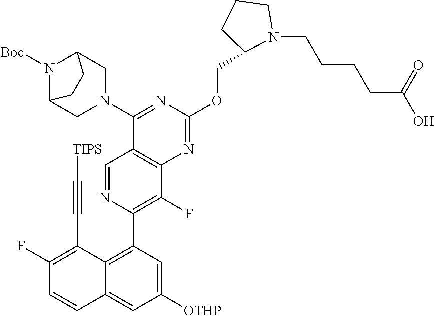

To a solution of tert-butyl 3-(2-(((S)-1-(5-ethoxy-5-oxopentyl)pyrrolidin-2-yl)methoxy)-8-fluoro-7-(7-fluoro-3-((tetrahydro-2H-pyran-2-yl)oxy)-8-((triisopropylsilyl)ethynyl)naphthalen-1-yl)pyrido[4,3-d]pyrimidin-4-yl)-3,8-diazabicyclo[3.2.1]octane-8-carboxylate (150 mg, 0.15 mmol, 1 eq) in tetrahydrofuran (1 mL), methanol (1 mL) and water (0.5 mL), was added lithium hydroxide (36 mg, 1.48 mmol, 10 eq). The mixture was stirred at 30° C. for 0.5 h. TLC (dichloromethane/methanol =10/1) showed the starting material was consumed completely and anew spot was detected. The reaction mixture was filtered and the filtrate was concentrated under reduced pressure to give a residue. The residue was purified by prep-TLC (dichloromethane/methanol=8/1) to afford 5-((2S)-2-(((4-(8-(tert-butoxycarbonyl)-3,8-diazabicyclo[3.2.1]octan-3-yl)-8-fluoro-7-(7-fluoro-3-((tetrahydro-2H-pyran-2-yl)oxy)-8-((triisopropylsilyl)ethynyl)naphthalen-1-yl)pyrido[4,3-d]pyrimidin-2-yl)oxy)methyl)pyrrolidin-1-yl)pentanoic acid (99 mg, 0.1 mmol, 68% yield) as a yellow oil.

Step 6: 5-((2S)-2-(((4-(8-(tert-butoxycarbonyl)-3,8-diazabicyclo[3.2.1]octan-3-yl)-7-(8-ethynyl-7-fluoro-3-((tetrahydro-2H-pyran-2-yl)oxy)naphthalen-1-yl)-8-fluoropyrido[4,3-d]pyrimidin-2-yl)oxy)methyl)pyrrolidin-1-yl)pentanoic acid

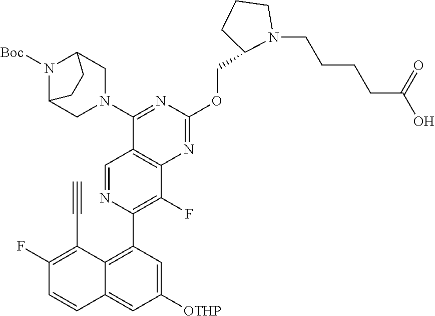

To a solution of 5-((2S)-2-(((4-(8-(tert-butoxycarbonyl)-3,8-diazabicyclo[3.2.1]octan-3-yl)-8-fluoro-7-(7-fluoro-3-((tetrahydro-2H-pyran-2-yl)oxy)-8-((triisopropylsilyl)ethynyl)naphthalen-1-yl)pyrido[4,3-d]pyrimidin-2-yl)oxy)methyl)pyrrolidin-1-yl)pentanoic acid (99 mg, 0.10 mmol, 1 eq) in N,N-dimethylformamide (10 mL) was added cesium fluoride (153 mg, 1.01 mmol, 10 eq). The mixture was stirred at 20° C. for 2 h. LCMS showed the desired mass was detected. The crude mixture was filtered and the filtrate was concentrated under reduced pressure to get a residue. The residue was purified by prep-TLC (dichloromethane/methanol=8/1) to afford 5-((2S)-2-(((4-(8-(tert-butoxycarbonyl)-3,8-diazabicyclo[3.2.1]octan-3-yl)-7-(8-ethynyl-7-fluoro-3-((tetrahydro-2H-pyran-2-yl)oxy)naphthalen-1-yl)-8-fluoropyrido[4,3-d]pyrimidin-2-yl)oxy)methyl)pyrrolidin-1-yl)pentanoic acid (66 mg, 0.08 mmol, 79% yield) as a yellow oil. LCMS (ESI, m/z): 827.1 [M+H]⁺.

Step 7: tert-butyl 3-(7-(8-ethynyl-7-fluoro-3-((tetrahydro-2H-pyran-2-yl)oxy)naphthalen-1-yl)-8-fluoro-2-(((S)-1-(5-(((S)-1-((2S,4R)-4-hydroxy-2-(((S)-1-(2′,3′,6′-trifluoro-[1,1′-biphenyl]-4-yl) ethyl)carbamoyl)pyrrolidin-1-yl)-3,3-dimethyl-1-oxobutan-2-yl)oxy)-5-oxopentyl)pyrrolidin-2-yl)methoxy)pyrido[4,3-d]pyrimidin-4-yl)-3,8-diazabicyclo[3.2.1]octane-8-carboxylate

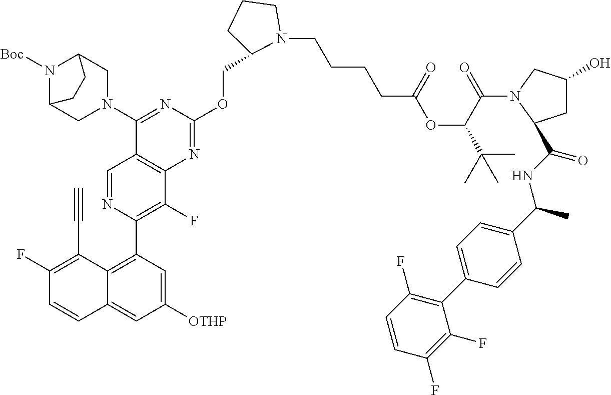

To a solution of 5-((2S)-2-(((4-(8-(tert-butoxycarbonyl)-3,8-diazabicyclo[3.2.1]octan-3-yl)-7-(8-ethynyl-7-fluoro-3-((tetrahydro-2H-pyran-2-yl)oxy)naphthalen-1-yl)-8-fluoropyrido[4,3-d]pyrimidin-2-yl)oxy)methyl)pyrrolidin-1-yl)pentanoic acid (66 mg, 0.08 mmol, 1 eq) in N,N-dimethylformamide (10 mL) was added (2S,4R)-1-((S)-2-amino-3,3-dimethylbutanoyl)-4-hydroxy-N—((S)-1-(2′,3′,6′-trifluoro-[1,1′-biphenyl]-4-yl)ethyl)pyrrolidine-2-carboxamide (57 mg, 0.12 mmol, 1.5 eq), N-(3-dimethylaminopropyl)-N-ethylcarbodiimide hydrochloride (38 mg, 0.20 mmol, 2.5 eq), diisopropylethylamine (103 mg, 0.80 mmol, 10 eq) and 1-hydroxybenzotriazole (21 mg, 0.16 mmol, 2 eq). The mixture was stirred at 20° C. for 12 h. LCMS showed the desired mass was detected. The reaction mixture was poured into ethyl acetate (20 mL). The resultant mixture was filtered and poured into water (20 mL). The organic phase was separated, washed with brine (20 mL×3), dried over anhydrous sodium sulfate, filtered and concentrated under reduced pressure to give a residue. The residue was purified by prep-TLC (dichloromethane/methanol=10/1) to afford tert-butyl 3-(7-(8-ethynyl-7-fluoro-3-((tetrahydro-2H-pyran-2-yl)oxy)naphthalen-1-yl)-8-fluoro-2-(((S)-1-(5-(((S)-1-((2S,4R)-4-hydroxy-2-(((S)-1-(2′,3′,6′-trifluoro-[1,1′-biphenyl]-4-yl)ethyl)carbamoyl)pyrrolidin-1-yl)-3,3-dimethyl-1-oxobutan-2-yl)oxy)-5-oxopentyl)pyrrolidin-2-yl)methoxy)pyrido[4,3-d]pyrimidin-4-yl)-3,8-diazabicyclo[3.2.1]octane-8-carboxylate (46 mg, 0.03 mmol, 45% yield) as a yellow oil. LCMS (ESI, m/z): 1286.5 [M]⁺

Step 8: (S)-1-((2S,4R)-4-hydroxy-2-(((S)-1-(2′,3′,6′-trifluoro-[1,1′-biphenyl]-4-yl)ethyl)carbamoyl) pyrrolidin-1-yl)-3,3-dimethyl-1-oxobutan-2-yl 5-((2S)-2-(((4-(3,8-diazabicyclo[3.2.1]octan-3-yl)-7-(8-ethynyl-7-fluoro-3-((tetrahydro-2H-pyran-2-yl)oxy)naphthalen-1-yl)-8-fluoropyrido[4,3-d]pyrimidin-2-yl)oxy)methyl)pyrrolidin-1-yl)pentanoate (Example 24)

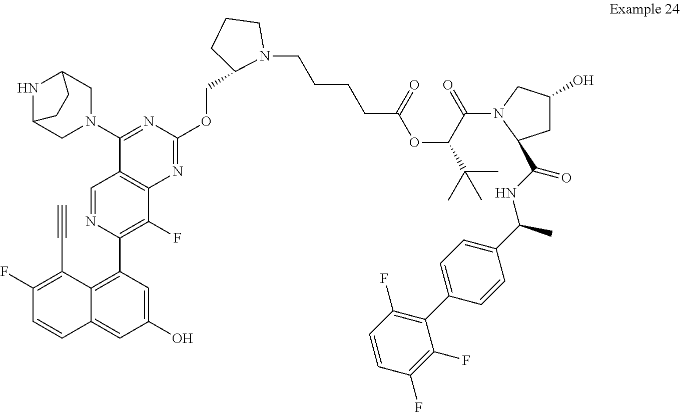 [SMILES: C#Cc1c(F)ccc2cc(O)cc(-c3ncc4c(N5CC6CCC(C5)N6)nc(OC[C@@H]5CCCN5CCCCC(=O)O[C@H](C(=O)N5C[C@H](O)C[C@H]5C(=O)N[C@@H](C)c5ccc(-c6c(F)ccc(F)c6F)cc5)C(C)(C)C)nc4c3F)c12]

To a solution of tert-butyl 3-(7-(8-ethynyl-7-fluoro-3-((tetrahydro-2H-pyran-2-yl)oxy) naphthalen-1-yl)-8-fluoro-2-(((S)-1-(5-(((S)-1-((2S,4R)-4-hydroxy-2-(((S)-1-(2′,3′,6′-trifluoro-[1,1′-biphenyl]-4-yl)ethyl)carbamoyl)pyrrolidin-1-yl)-3,3-dimethyl-1-oxobutan-2-yl)oxy)-5-oxopentyl) pyrrolidin-2-yl)methoxy)pyrido[4,3-d]pyrimidin-4-yl)-3,8-diazabicyclo[3.2.1]octane-8-carboxylate (40 mg, 0.03 mmol, 1 eq) in dichloromethane (3 mL) was added trifluoroacetic acid (1 mL). The mixture was stirred at 20° C. for 0.5 h. LCMS showed the desired mass was detected. The reaction mixture was filtered, and the filtrate was concentrated under reduced pressure to give a residue. The residue was purified by prep-HPLC (column: Phenomenex Synergi C18 150*25 mm*10 um; mobile phase: [water(FA)-ACN]; B %: 17%-37%, 10 min) to afford the desired product (S)-1-((2S,4R)-4-hydroxy-2-(((S)-1-(2′,3′,6′-trifluoro-[1,1′-biphenyl]-4-yl)ethyl)carbamoyl)pyrrolidin-1-yl)-3,3-dimethyl-1-oxobutan-2-yl 5-((2S)-2-(((4-(3,8-diazabicyclo[3.2.1]octan-3-yl)-7-(8-ethynyl-7-fluoro-3-((tetrahydro-2H-pyran-2-yl)oxy)naphthalen-1-yl)-8-fluoropyrido[4,3-d]pyrimidin-2-yl)oxy)methyl)pyrrolidin-1-yl)pentanoate (8 mg, 0.007 mmol, 23% yield) as a yellow solid. LCMS (ESI, m/z): 1102.5 [M]⁺. ¹H NMR (400 MHz, DMSO-d₆) 9.04 (s, 1H), 8.40 (d, J=7.6 Hz, 1H), 8.29 (s, 2H), 8.04-7.93 (m, 1H), 7.78 (d, J=8.8 Hz, 1H), 7.54 (dd, J=4.8, 9.2 Hz, 1H), 7.50-7.45 (m, 1H), 7.43 (s, 4H), 7.39 (d, J=2.4 Hz, 1H), 7.29-7.24 (m, 1H), 7.18 (d, J=2.4 Hz, 1H), 4.98-4.88 (m, 1H), 4.54-4.48 (m, 2H), 4.44-4.37 (m, 2H), 4.32-4.24 (m, 2H), 4.11-4.06 (m, 1H), 3.96-3.91 (m, 1H), 3.72-3.63 (m, 2H), 3.07-3.01 (m, 1H), 2.88-2.74 (m, 2H), 2.31-2.22 (m, 2H), 2.21-2.07 (m, 3H), 2.05-1.99 (m, 1H), 1.94-1.88 (m, 1H), 1.84-1.77 (m, 1H), 1.75-1.54 (m, 9H), 1.47-1.35 (m, 7H), 0.92 (d, J=3.6 Hz, 11H).

Example 25: (2S,4R)-1-((2S)-2-(3-(3-((2S)-2-(((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)pyrrolidin-1-yl)propoxy)propanamido)-3,3-dimethylbutanoyl)-N—((S)-1-(2′-chloro-[1,1′-biphenyl]-4-yl)ethyl)-4-hydroxypyrrolidine-2-carboxamide

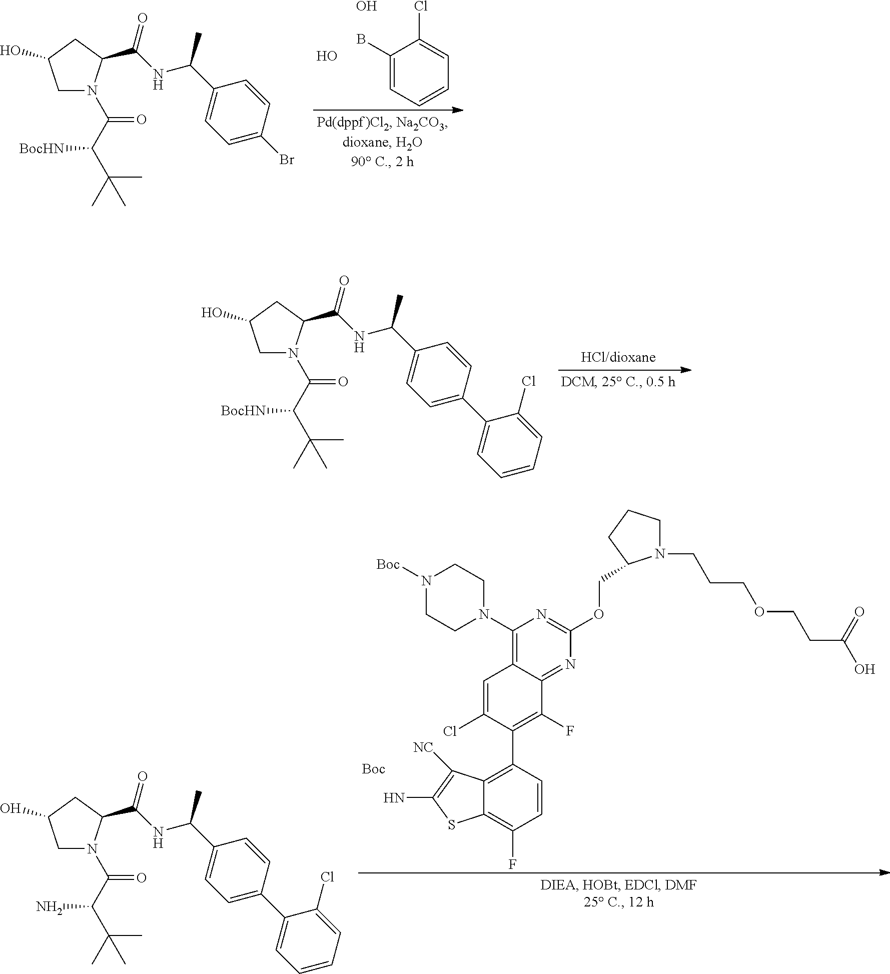

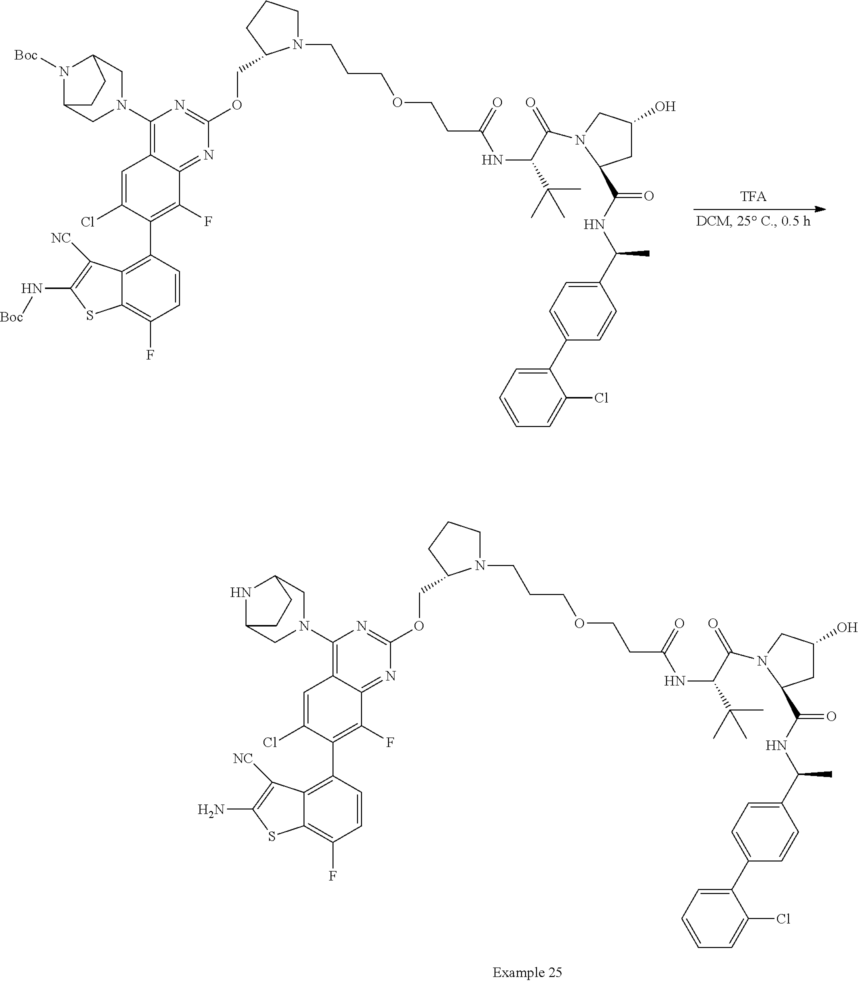 [SMILES: C[C@H](NC(=O)[C@@H]1C[C@@H](O)CN1C(=O)[C@@H](NC(=O)CCOCCCN1CCC[C@H]1COc1nc(N2CC3CCC(C2)N3C(=O)OC(C)(C)C)c2cc(Cl)c(-c3ccc(F)c4sc(NC(=O)OC(C)(C)C)c(C#N)c34)c(F)c2n1)C(C)(C)C)c1ccc(-c2ccccc2Cl)cc1 ; C[C@H](NC(=O)[C@@H]1C[C@@H](O)CN1C(=O)[C@@H](NC(=O)CCOCCCN1CCC[C@H]1COc1nc(N2CC3CCC(C2)N3)c2cc(Cl)c(-c3ccc(F)c4sc(N)c(C#N)c34)c(F)c2n1)C(C)(C)C)c1ccc(-c2ccccc2Cl)cc1]

Step 1: tert-butyl ((S)-1-((2S,4R)-2-(((S)-1-(2′-chloro-[1,1′-biphenyl]-4-yl)ethyl)carbamoyl)-4-hydroxypyrrolidin-1-yl)-3,3-dimethyl-1-oxobutan-2-yl)carbamate

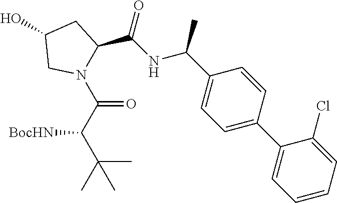 [SMILES: C[C@H](NC(=O)[C@@H]1C[C@@H](O)CN1C(=O)[C@@H](NC(=O)OC(C)(C)C)C(C)(C)C)c1ccc(-c2ccccc2Cl)cc1]

A mixture of tert-butyl ((S)-1-((2S,4R)-2-(((S)-1-(4-bromophenyl)ethyl)carbamoyl)-4-hydroxypyrrolidin-1-yl)-3,3-dimethyl-1-oxobutan-2-yl)carbamate (500 mg, 0.95 mmol), (2-chlorophenyl)boronic acid (223 mg, 1.42 mmol), 1,1′-bis(diphenylphosphino)ferrocene]dichloropalladium(II) (69 mg, 0.09 mmol), sodium carbonate (302 mg, 2.85 mmol) in dioxane (10 mL), water (2 mL) was degassed and purged with nitrogen for 3 times, and then the mixture was stirred at 90° C. for 2 h under nitrogen atmosphere. The reaction mixture was quenched by adding water (50 mL), and then extracted with ethyl acetate (50 mL×2). The combined organic layers were washed with brine (100 mL), dried over sodium sulfate, filtered and concentrated under reduced pressure to give a residue. The residue was purified by prep-HPLC (column: Phenomenex luna C18 150*40 mm*15 um; mobile phase: [water(FA)-ACN]; B %: 53%-83%, 1 0 min) to afford the desired product tert-butyl ((S)-1-((2S,4R)-2-(((S)-1-(2′-chloro-[1,1′-biphenyl]-4-yl)ethyl)carbamoyl)-4-hydroxypyrrolidin-1-yl)-3,3-dimethyl-1-oxobutan-2-yl)carbamate (320 mg, 0.57 mmol, 60% yield) as a white solid. ¹H NMR (400 MHz, CHLOROFORM-d) δ 8.42 (br d, J=7.6 Hz, 1H), 7.58-7.54 (m, 1H), 7.45-7.41 (m, 2H), 7.40-7.34 (m, 5H), 6.43 (br d, J=8.8 Hz, 1H), 5.14 (br s, 1H), 4.98-4.90 (m, 1H), 4.46 (br t, J=8.0 Hz, 1H), 4.30 (br s, 1H), 4.15 (br d, J=9.2 Hz, 1H), 3.64-3.55 (m, 2H), 2.08-2.00 (m, 1H), 1.84-1.76 (m, 1H), 1.41-1.36 (m, 12H), 0.94 (s, 9H)

Step 2: (2S,4R)-1-((S)-2-amino-3,3-dimethylbutanoyl)-N—((S)-1-(2′-chloro-[1,1′-biphenyl]-4-yl)ethyl)-4-hydroxypyrrolidine-2-carboxamide

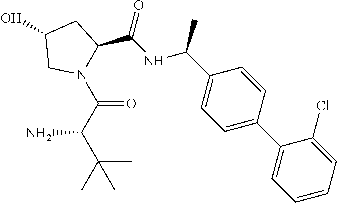 [SMILES: C[C@H](NC(=O)[C@@H]1C[C@@H](O)CN1C(=O)[C@@H](N)C(C)(C)C)c1ccc(-c2ccccc2Cl)cc1]

To a solution of tert-butyl ((S)-1-((2S,4R)-2-(((S)-1-(2′-chloro-[1,1′-biphenyl]-4-yl)ethyl)carbamoyl)-4-hydroxypyrrolidin-1-yl)-3,3-dimethyl-1-oxobutan-2-yl)carbamate (120 mg, 0.21 mmol) in dichloromethane (2 mL) was added hydrochloric acid/dioxane (4 M, 2 mL). The mixture was stirred at 25° C. for 0.5 h. The reaction mixture was concentrated under reduced pressure to remove solvent to afford the crude product (2S,4R)-1-((S)-2-amino-3,3-dimethylbutanoyl)-N—((S)-1-(2′-chloro-[1,1′-biphenyl]-4-yl)ethyl)-4-hydroxypyrrolidine-2-carboxamide (98 mg, 0.21 mmol, 99% yield) as a yellow oil. LCMS (ESI, m/z): 458.1 [M]⁺.

Step 3: tert-butyl 3-(7-(2-((tert-butoxycarbonyl)amino)-3-cyano-7-fluorobenzo[b]thiophen-4-yl)-6-chloro-2-(((S)-1-(3-(3-(((S)-1-((2S,4R)-2-(((S)-1-(2′-chloro-[1,1′-biphenyl]-4-yl)ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-8-fluoroquinazolin-4-yl)-3,8-diazabicyclo[3.2.1]octane-8-carboxylate

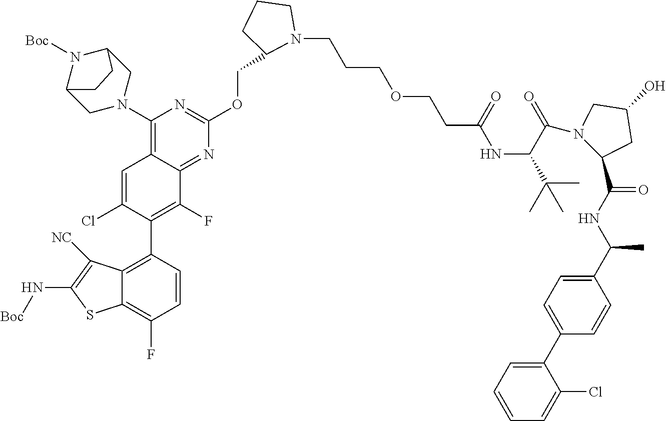

To a solution of 3-(3-((2S)-2-(((4-(8-(tert-butoxycarbonyl)-3,8-diazabicyclo[3.2.1]octan-3-yl)-7-(2-((tert-butoxycarbonyl)amino)-3-cyano-7-fluorobenzo[b]thiophen-4-yl)-6-chloro-8-fluoroquinazolin-2-yl)oxy)methyl)pyrrolidin-1-yl)propoxy)propanoic acid (120 mg, 0.13 mmol, 1 eq), (2S,4R)-1-((S)-2-amino-3,3-dimethylbutanoyl)-N—((S)-1-(2′-chloro-[1,1′-biphenyl]-4-yl)ethyl)-4-hydroxypyrrolidine-2-carboxamide (98 mg, 0.21 mmol, hydrochloride) in dimethylformamide (2 mL) was added diisopropylethylamine (85 mg, 0.66 mmol, 0.1 mL), 1-hydroxybenzotriazole (53 mg, 0.39 mmol) and 1-(3-dimethylaminopropyl)-3-ethylcarbodiimide hydrochloride (76 mg, 0.39 mmol). The mixture was stirred at 25° C. overnight. The reaction mixture was filtered and the filtrate was purified by prep-HPLC(column: Phenomenex Synergi Polar-RP 100*25 mm*4 um; mobile phase: [water(TFA)-ACN]; B %: 67%-87%, 7 min) to afford the desired product tert-butyl 3-(7-(2-((tert-butoxycarbonyl)amino)-3-cyano-7-fluorobenzo[b]thiophen-4-yl)-6-chloro-2-(((S)-1-(3-(3-(((S)-1-((2S,4R)-2-(((S)-1-(2′-chloro-[1,1′-biphenyl]-4-yl)ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-8-fluoroquinazolin-4-yl)-3,8-diazabicyclo[3.2.1]octane-8-carboxylate (100 mg, 0.07 mmol, 56% yield) as a yellow solid. LCMS (ESI, m/z): 1352.4 [M]⁺

Step 4: (2S,4R)-1-((2S)-2-(3-(3-((2S)-2-(((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)pyrrolidin-1-yl)propoxy)propanamido)-3,3-dimethylbutanoyl)-N—((S)-1-(2′-chloro-[1,1′-biphenyl]-4-yl)ethyl)-4-hydroxypyrrolidine-2-carboxamide (Example 25)

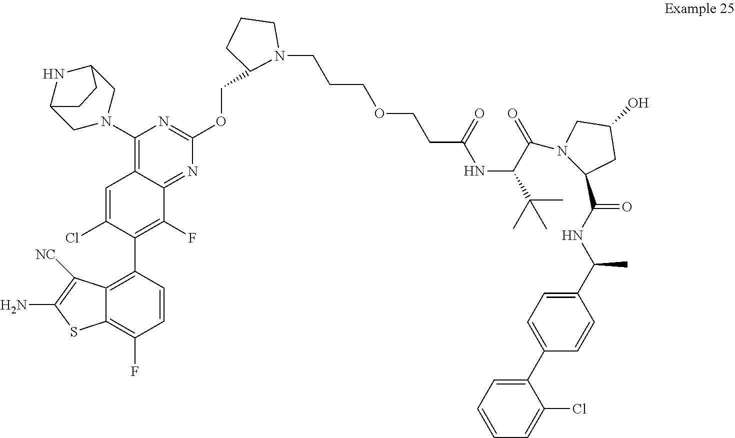

To a solution of tert-butyl 3-(7-(2-((tert-butoxycarbonyl)amino)-3-cyano-7-fluorobenzo[b]thiophen-4-yl)-6-chloro-2-(((S)-1-(3-(3-(((S)-1-((2S,4R)-2-(((S)-1-(2′-chloro-[1,1′-biphenyl]-4-yl)ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-8-fluoroquinazolin-4-yl)-3,8-diazabicyclo[3.2.1]octane-8-carboxylate (100 mg, 0.07 mmol) in dichloromethane (2 mL) was added trifluoroacetic acid (3.08 g, 27.01 mmol, 2 mL). The mixture was stirred at 25° C. for 0.5 h. The reaction mixture was concentrated under reduced pressure to remove solvent to give a residue. The residue was purified by prep-HPLC (column: Phenomenex luna C18 150*25 mm*10 um; mobile phase: [water(FA)-ACN]; B %: 16%-46%, 10 min) to afford the desired product (2S,4R)-1-((2S)-2-(3-(3-((2S)-2-(((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)pyrrolidin-1-yl)propoxy)propanamido)-3,3-dimethylbutanoyl)-N—((S)-1-(2′-chloro-[1,1′-biphenyl]-4-yl)ethyl)-4-hydroxypyrrolidine-2-carboxamide (31.7 mg, 0.03 mmol, 37% yield) as a yellow solid. LCMS (ESI, m/z): 1151.4 [M]⁺, ¹H NMR (400 MHz, DMSO-d6) δ 8.37 (br d, J=7.6 Hz, 1H), 8.24 (s, 2H), 8.10 (s, 2H), 7.85-7.80 (m, 2H), 7.57-7.54 (m, 1H), 7.42-7.38 (m, 3H), 7.38-7.36 (m, 4H), 7.26 (dd, J=5.2, 8.4 Hz, 1H), 7.17-7.12 (m, 1H), 4.94 (t, J=7.6 Hz, 1H), 4.52 (d, J=9.6 Hz, 1H), 4.43 (t, J=8.0 Hz, 1H), 4.36-4.32 (m, 1H), 4.32-4.24 (m, 3H), 4.05 (td, J=6.8, 10.8 Hz, 1H), 3.63-3.59 (m, 3H), 3.02 (br d, J=4.0 Hz, 1H), 2.92-2.80 (m, 4H), 2.37 (br dd, J=5.6, 7.6 Hz, 2H), 2.31-2.23 (m, 2H), 2.22-2.14 (m, 2H), 2.05-1.97 (m, 2H), 1.93-1.87 (m, 1H), 1.85-1.77 (m, 2H), 1.73-1.58 (m, 12H), 1.38 (d, J=7.2 Hz, 3H), 0.91 (br d, J=2.8 Hz, 9H).

Example 26: (2S,4R)-1-((2S)-2-(3-(3-((2S)-2-(((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)pyrrolidin-1-yl)propoxy)propanamido)-3,3-dimethylbutanoyl)-4-hydroxy-N—((S)-1-(2′-methyl-[1,1′-biphenyl]-4-yl)ethyl)pyrrolidine-2-carboxamide

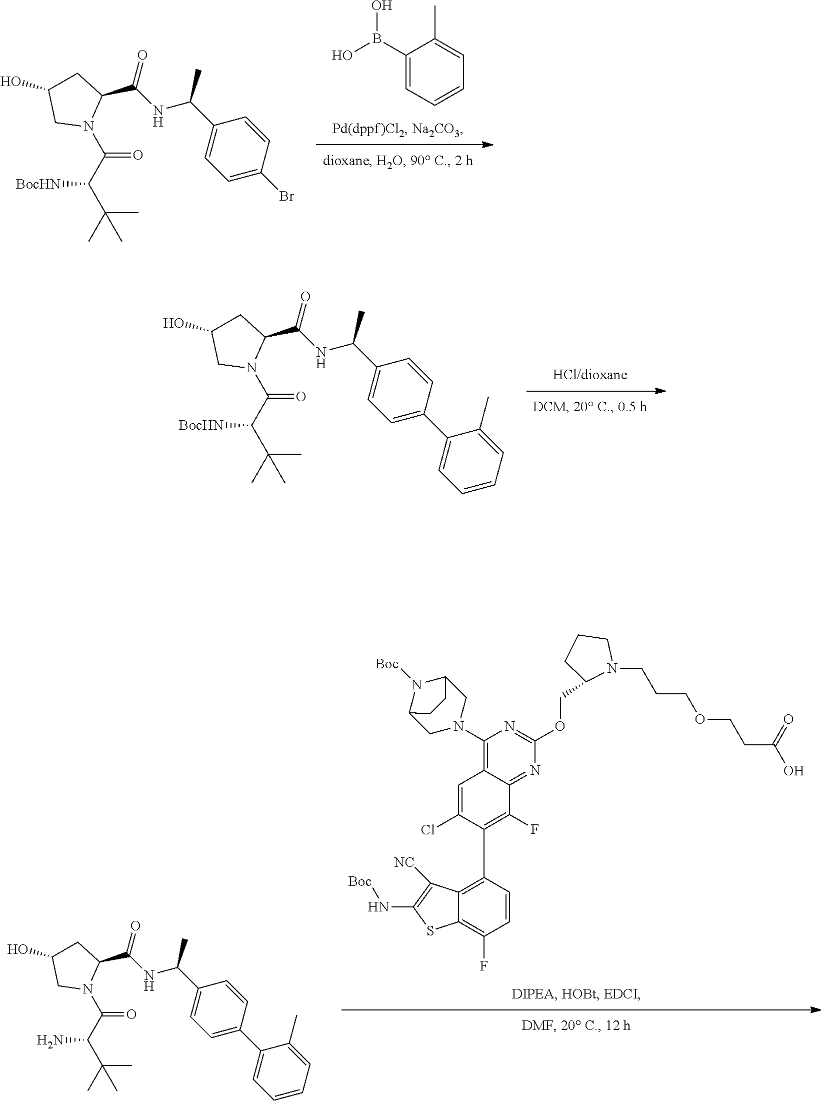

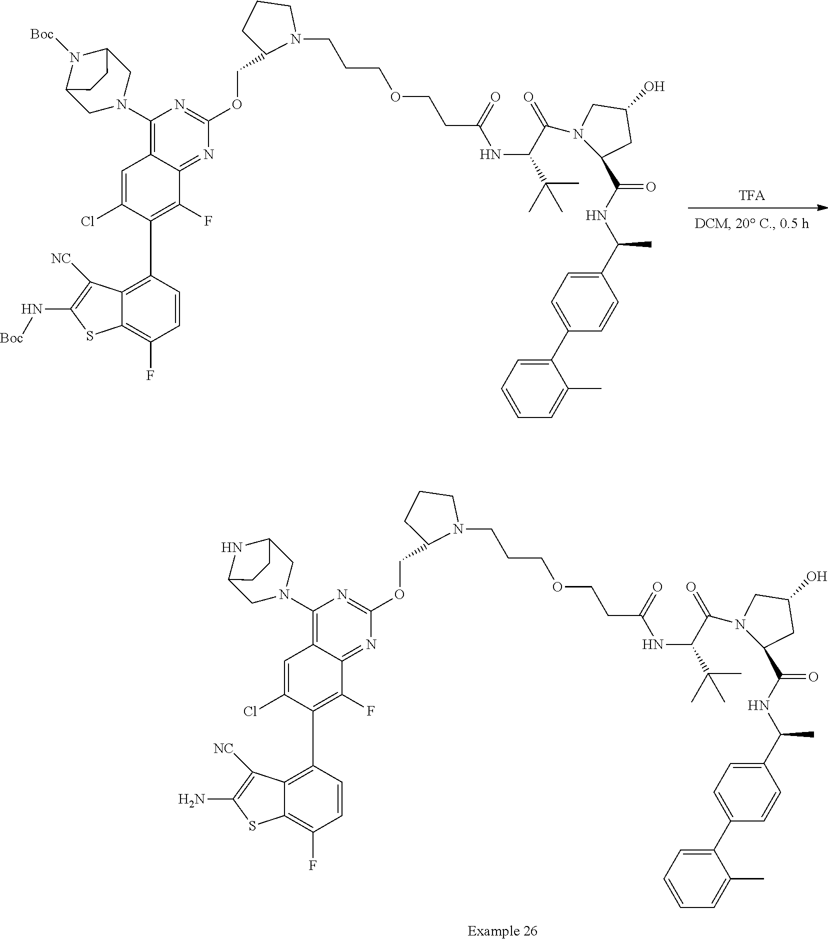

Step 1: tert-butyl ((S)-1-((2S,4R)-4-hydroxy-2-(((S)-1-(2′-methyl-[1,1′-biphenyl]-4-yl)ethyl)carbamoyl)pyrrolidin-1-yl)-3,3-dimethyl-1-oxobutan-2-yl)carbamate

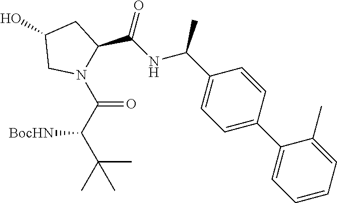 [SMILES: Cc1ccccc1-c1ccc([C@H](C)NC(=O)[C@@H]2C[C@@H](O)CN2C(=O)[C@@H](NC(=O)OC(C)(C)C)C(C)(C)C)cc1]

A mixture of tert-butyl ((S)-1-((2S,4R)-2-(((S)-1-(4-bromophenyl)ethyl)carbamoyl)-4-hydroxypyrrolidin-1-yl)-3,3-dimethyl-1-oxobutan-2-yl)carbamate (1 g, 1.9 mmol, 1 eq), o-tolylboronic acid (387 mg, 2.85 mmol, 1.5 eq), cyclopentyl(diphenyl)phosphane; dichloropalladium; iron (139 mg, 0.19 mmol, 0.1 eq), sodium carbonate (604 mg, 5.7 mmol, 3 eq) in dioxane (10 mL) and water (2 mL) was degassed and purged with nitrogen for 3 times, and then the mixture was stirred at 90° C. for 2 h under nitrogen atmosphere. LCMS showed the starting material was consumed completely and one main peak with desired mass was detected. Water (20 mL) was added before the mixture was extracted by ethyl acetate(20 mL×3). The combined organic layers were washed with brine (20 mL×3), dried over anhydrous sodium sulfate, filtered and concentrated under reduced pressure to give a residue. The residue was purified by prep-TLC (dichloromethane/methanol=10/1) to afford the desired compound (1.3 g, crude) as a white solid. LCMS (ESI, m/z): 538.6 [M+1]⁺

Step 2: (2S,4R)-1-((S)-2-amino-3,3-dimethylbutanoyl)-4-hydroxy-N—((S)-1-(2′-methyl-[1,1′-biphenyl]-4-yl)ethyl)pyrrolidine-2-carboxamide

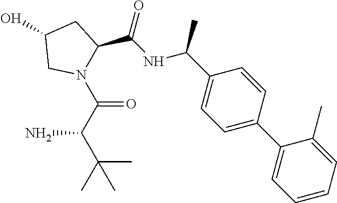 [SMILES: Cc1ccccc1-c1ccc([C@H](C)NC(=O)[C@@H]2C[C@@H](O)CN2C(=O)[C@@H](N)C(C)(C)C)cc1]

To a solution of tert-butyl ((S)-1-((2S,4R)-4-hydroxy-2-(((S)-1-(2′-methyl-[1,1′-biphenyl]-4-yl)ethyl)carbamoyl)pyrrolidin-1-yl)-3,3-dimethyl-1-oxobutan-2-yl)carbamate (100 mg, 0.18 mmol, 1 eq) in dichloromethane (5 mL) was added hydrogen chloride/dioxane (4 M, 0.1 mL, 3 eq). The mixture was stirred at 20° C. for 0.5 h. LCMS showed the starting material was consumed completely and one main peak with desired mass was detected. The reaction mixture was concentrated under reduced pressure to remove solvent. The product (2S,4R)-1-((S)-2-amino-3,3-dimethylbutanoyl)-4-hydroxy-N—((S)-1-(2′-methyl-[1,1′-biphenyl]-4-yl)ethyl)pyrrolidine-2-carboxamide (82 mg, crude product) was obtained as a yellow solid, and the crude product was used into the next step without further purification. LCMS (ESI, m/z): 438.4 [M+1]⁺.

Step 3: tert-butyl 3-(7-(2-((tert-butoxycarbonyl)amino)-3-cyano-7-fluorobenzo[b]thiophen-4-yl)-6-chloro-8-fluoro-2-(((S)-1-(3-(3-(((S)-1-((2S,4R)-4-hydroxy-2-(((S)-1-(2′-methyl-[1,1′-biphenyl]-4-yl)ethyl)carbamoyl)pyrrolidin-1-yl)-3,3-dimethyl-1-oxobutan-2-yl)amino)-3-oxopropoxy)propyl)pyrrolidin-2-yl)methoxy)quinazolin-4-yl)-3,8-diazabicyclo[3.2.1]octane-8-carboxylate

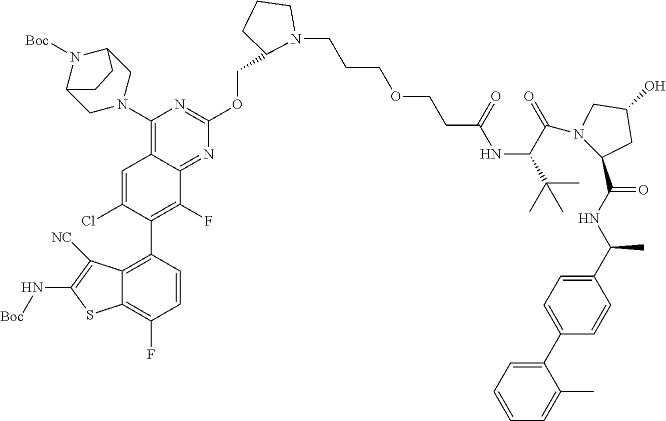

To a solution of (2S,4R)-1-((S)-2-amino-3,3-dimethylbutanoyl)-4-hydroxy-N—((S)-1-(2′-methyl-[1,1′-biphenyl]-4-yl)ethyl)pyrrolidine-2-carboxamide (52 mg, 0.1 mmol, 1 eq, hydrochloride) and 3-(3-((2S)-2-(((4-(8-(tert-butoxycarbonyl)-3,8-diazabicyclo[3.2.1]octan-3-yl)-7-(2-((tert-butoxycarbonyl)amino)-3-cyano-7-fluorobenzo[b]thiophen-4-yl)-6-chloro-8-fluoroquinazolin-2-yl)oxy)methyl)pyrrolidin-1-yl)propoxy)propanoic acid (100 mg, 0.1 mmol, 1 eq) in N,N-dimethylformamide (3 mL) was added N,N-diisopropylethylamine (42.5 mg, 0.3 mmol, 3 eq), 1-hydroxybenzotriazole (29.6 mg, 0.2 mmol, 2 eq) and N-(3-dimethylaminopropyl)-N-ethylcarbodiimide hydrochloride (42 mg, 0.22 mmol, 2 eq). The mixture was stirred at 20° C. for 12 h. TLC (dichloromethane/methanol=10/1) and LCMS showed the starting material was consumed completely and one main peak with desired mass was detected. Water (20 mL) was added before the mixture was extracted by ethyl acetate(20 mL×3). The combined organic layers were washed with brine (20 mL×3), dried over anhydrous sodium sulfate, filtered and the filtrate was concentrated under reduced pressure to give a residue. The residue was purified by prep-TLC (dichloromethane/methanol=10/1) to afford the compound (68 mg, 0.05 mmol, 43% yield) as a yellow solid. LCMS (ESI, m/z): 1330.5 [M]⁺.

Step 4: (2S,4R)-1-((2S)-2-(3-(3-((2S)-2-(((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)pyrrolidin-1-yl)propoxy)propanamido)-3,3-dimethylbutanoyl)-4-hydroxy-N—((S)-1-(2′-methyl-[1,1′-biphenyl]-4-yl)ethyl)pyrrolidine-2-carboxamide (Example 26)

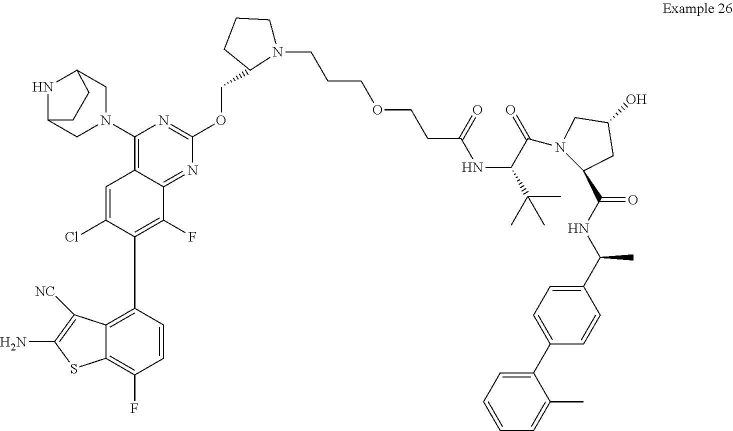

To a solution of tert-butyl 3-(7-(2-((tert-butoxycarbonyl)amino)-3-cyano-7-fluorobenzo[b]thiophen-4-yl)-6-chloro-8-fluoro-2-(((S)-1-(3-(3-(((S)-1-((2S,4R)-4-hydroxy-2-(((S)-1-(2′-methyl-[1,1′-biphenyl]-4-yl)ethyl)carbamoyl)pyrrolidin-1-yl)-3,3-dimethyl-1-oxobutan-2-yl)amino)-3-oxopropoxy)propyl)pyrrolidin-2-yl)methoxy)quinazolin-4-yl)-3,8-diazabicyclo[3.2.1]octane-8-carboxylate (68 mg, 0.05 mmol, 1 eq) in dichloromethane (3 mL) was added trifluoroacetic acid (1 mL). The mixture was stirred at 20° C. for 0.5 h. LCMS showed the starting material was consumed completely and one main peak with desired mass was detected. The reaction mixture was bubbled by nitrogen gas to remove dichloromethane. The residue was purified by prep-HPLC (column: Phenomenex Synergi C18 150*25 mm*10 um; mobile phase: [water (formic acid)-acetonitrile]; B %: 20%-40%, 10 min) to afford the desired product (39 mg, 0.03 mmol, 64% yield, formate[1]) as a white solid. LCMS (ESI, m/z): 1131.4 [M]⁺, ¹H NMR (400 MHz, DMSO-d₆) δ=8.34 (d, J=8.0 Hz, 1H), 8.13 (d, J=5.2 Hz, 2H), 7.92 (s, 1H), 7.83 (d, J=8.4 Hz, 1H), 7.36-7.31 (m, 2H), 7.30-7.21 (m, 7H), 7.17 (d, J=8.8 Hz, 2H), 5.14-5.12 (m, 1H), 5.15-4.87 (m, 1H), 4.56-4.45 (m, 3H), 4.45-4.32 (m, 3H), 4.29 (s, 1H), 4.16-4.08 (m, 2H), 3.87-3.82 (m, 1H), 3.68 (d, J=12.8 Hz, 1H), 3.65-3.46 (m, 6H), 3.41 (d, J=7.2 Hz, 4H), 2.22 (s, 3H), 2.09-1.96 (m, 3H), 1.96-1.69 (m, 12H), 1.38 (d, J=6.8 Hz, 3H), 0.91 (d, J=2.4 Hz, 9H).

Example 27: (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)propanamido)-3,3-dimethylbutanoyl)-N—((S)-1-(4-(3,5-difluoropyridin-4-yl)phenyl)ethyl)-4-hydroxypyrrolidine-2-carboxamide

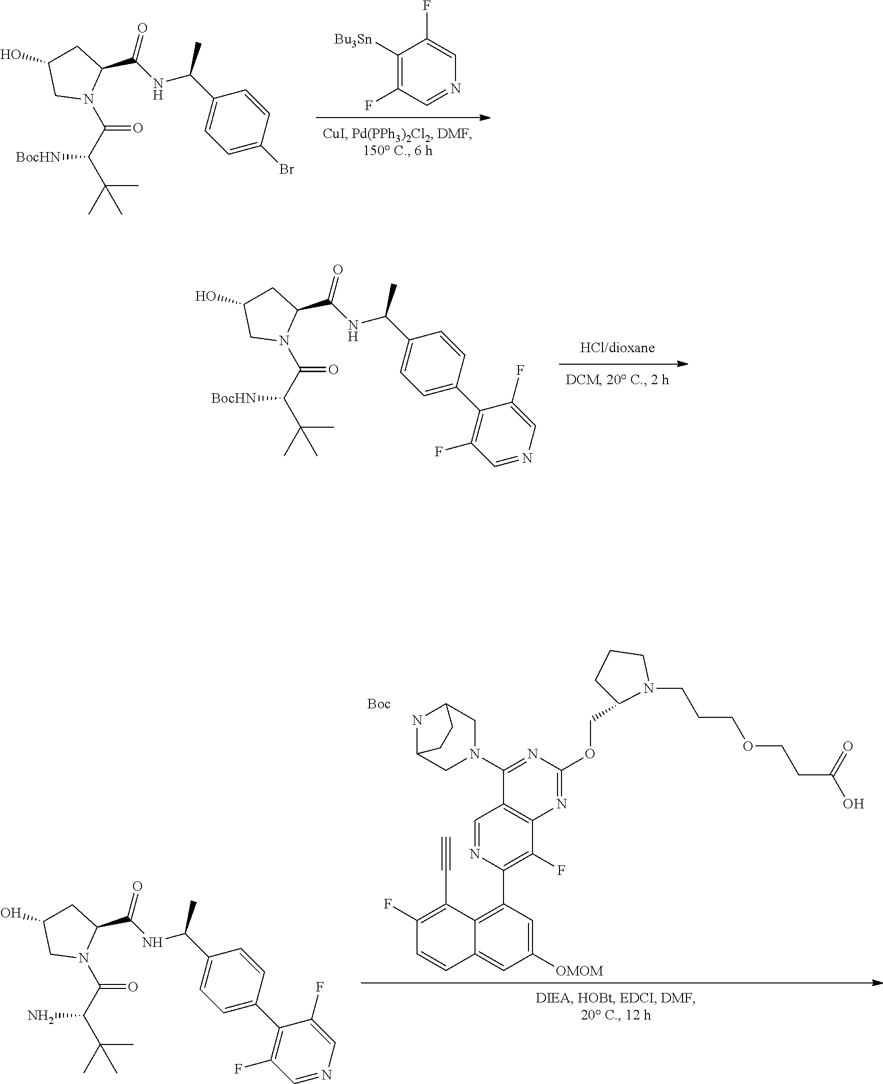

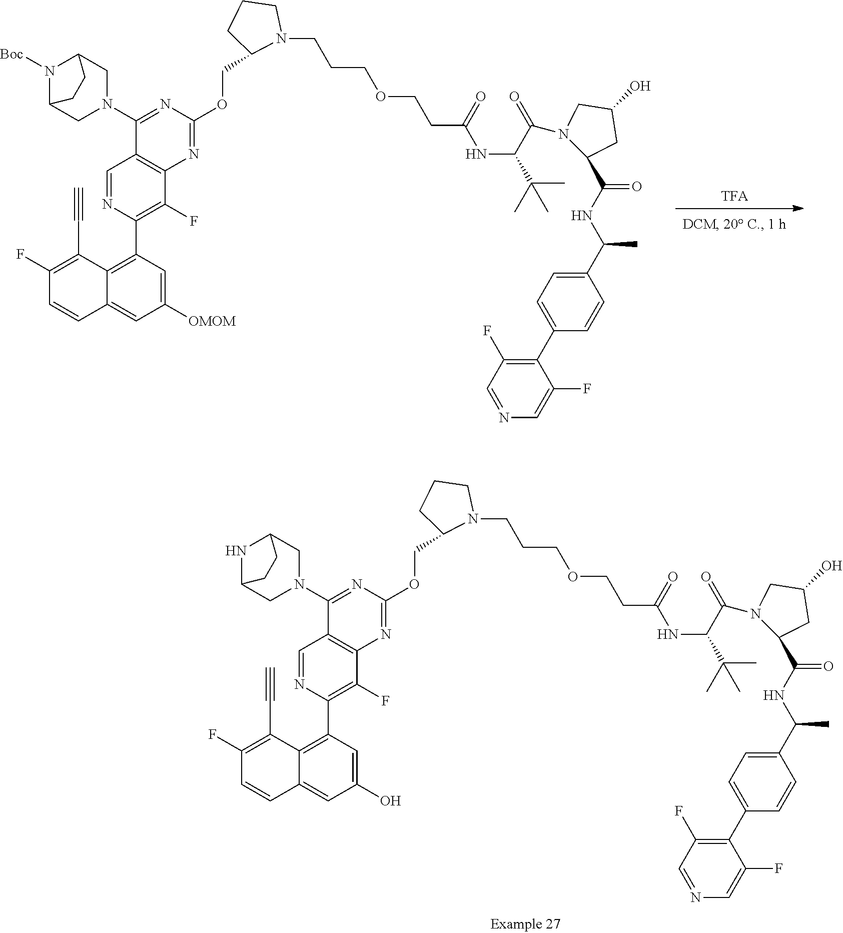

Step 1: tert-butyl ((S)-1-((2S,4R)-2-(((S)-1-(4-(3,5-difluoropyridin-4-yl)phenyl)ethyl)carbamoyl)-4-hydroxypyrrolidin-1-yl)-3,3-dimethyl-1-oxobutan-2-yl)carbamate

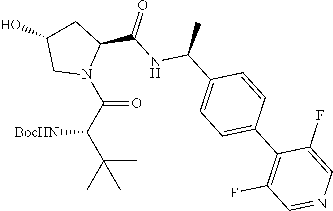 [SMILES: C[C@H](NC(=O)[C@@H]1C[C@@H](O)CN1C(=O)[C@@H](NC(=O)OC(C)(C)C)C(C)(C)C)c1ccc(-c2c(F)cncc2F)cc1]

tert-butyl N-[(1S)-1-[(2S,4R)-2-[[(1S)-1-(4-bromophenyl)ethyl]carbamoyl]-4-hydroxy-pyrrolidine-1-carbonyl]-2,2-dimethylpropyl]carbamate (500 mg, 0.95 mmol, 1 eq), 3,5-difluoro-4-(tributylstannyl)pyridine (422 mg, 1.04 mmol, 1.1 eq) cuprous iodide (36 mg, 0.19 mmol, 0.2 eq), and [1,1′-bis(diphenylphosphino)ferrocene]dichloropalladium(II) (66 mg, 0.10 mmol, 0.1 eq) were taken up into a microwave tube in N,N-dimethylformamide (5 mL). The sealed tube was heated at 150° C. for 6 h under microwave. LCMS showed the desired mass was detected. The reaction mixture was filtered, diluted with water (40 mL) and extracted with ethyl acetate (20 mL×3). The combined organic layers were washed with brine (15 mL×3), dried over anhydrous sodium sulfate, filtered and concentrated under reduced pressure to give a residue. The residue was purified by silica gel chromatography (petroleum ether/ethyl acetate=1/0 to 1/1) to afford the product tert-butyl ((S)-1-((2S,4R)-2-(((S)-1-(4-(3,5-difluoropyridin-4-yl)phenyl)ethyl)carbamoyl)-4-hydroxypyrrolidin-1-yl)-3,3-dimethyl-1-oxobutan-2-yl)carbamate (360 mg, 0.64 mmol, 24% yield) as a white solid. LCMS (ESI, m/z): 560.63 [M+H]⁺.

Step 2: (2S,4R)-1-((S)-2-amino-3,3-dimethylbutanoyl)-N—((S)-1-(4-(3,5-difluoropyridin-4-yl)phenyl)ethyl)-4-hydroxypyrrolidine-2-carboxamide

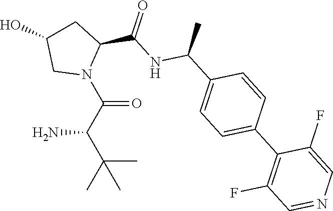 [SMILES: C[C@H](NC(=O)[C@@H]1C[C@@H](O)CN1C(=O)[C@@H](N)C(C)(C)C)c1ccc(-c2c(F)cncc2F)cc1]

To a solution of tert-butyl ((S)-1-((2S,4R)-2-(((S)-1-(4-(3,5-difluoropyridin-4-yl)phenyl)ethyl) carbamoyl)-4-hydroxypyrrolidin-1-yl)-3,3-dimethyl-1-oxobutan-2-yl)carbamate (150 mg, 0.26 mmol, 1 eq) in dichloromethane (3 mL) was added hydrochloride/dioxane (3 mL). The mixture was stirred at 20° C. for 2 h. LCMS showed the desired mass was detected. The reaction mixture was filtered and concentrated under reduced pressure to afford the crude product (2S,4R)-1-((S)-2-amino-3,3-dimethylbutanoyl)-N—((S)-1-(4-(3,5-difluoropyridin-4-yl)phenyl)ethyl)-4-hydroxypyrrolidine-2-carboxamide (120 mg, 0.26 mmol, 97% yield) as a colorless oil, which was used into the next step directly. LCMS (ESI, m/z): 461.3 [M+H]⁺.

Step 3: tert-butyl 3-(2-(((S)-1-(3-(3-(((S)-1-((2S,4R)-2-(((S)-1-(4-(3,5-difluoropyridin-4-yl)phenyl) ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-7-(8-ethynyl-7-fluoro-3-(methoxymethoxy) naphthalen-1-yl)-8-fluoropyrido[4,3-d]pyrimidin-4-yl)-3,8-diazabicyclo[3.2.1]octane-8-carboxylate

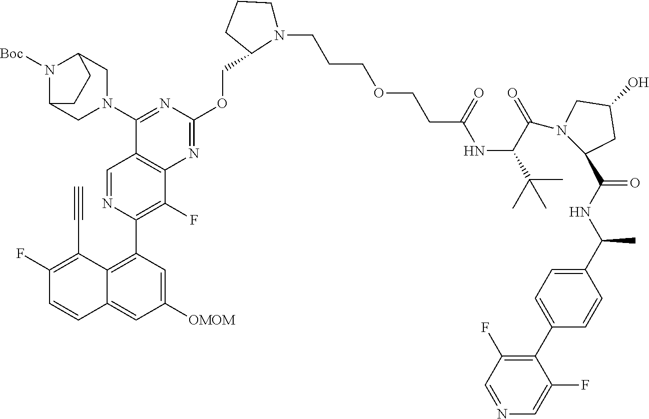

To a solution of 3-(3-((2S)-2-(((4-(8-(tert-butoxycarbonyl)-3,8-diazabicyclo[3.2.1]octan-3-yl)-7-(8-ethynyl-7-fluoro-3-(methoxymethoxy)naphthalen-1-yl)-8-fluoropyrido[4,3-d]pyrimidin-2-yl)oxy)methyl)pyrrolidin-1-yl)propoxy)propanoic acid (90 mg, 0.11 mmol, 1 eq) in N,N-dimethylformamide (2 mL) was added 1-hydroxybenzotriazole (30 mg, 0.22 mmol, 2 eq), (2S,4R)-1-((S)-2-amino-3,3-dimethylbutanoyl)-N—((S)-1-(4-(3,5-difluoropyridin-4-yl)phenyl) ethyl)-4-hydroxypyrrolidine-2-carboxamide (100 mg, 0.20 mmol, 2 eq, hydrogen chloride), N-(3-dimethylaminopropyl)-N-ethylcarbodiimide hydrochloride (42 mg, 0.22 mmol, 2 eq) and diisopropylethylamine (71 mg, 0.55 mmol, 5 eq). The mixture was stirred at 20° C. for 12 h. LCMS showed the desired mass was detected. The reaction mixture was partitioned between water (40 mL) and ethyl acetate (40 mL×3). The organic phase was separated, washed with brine (50 mL), dried over anhydrous sodium sulfate, filtered and concentrated under reduced pressure to give a residue. The residue was purified by prep-TLC (dichloromethane/methanol=10/1) to afford the crude product tert-butyl 3-(2-(((S)-1-(3-(3-(((S)-1-((2S,4R)-2-(((S)-1-(4-(3,5-difluoropyridin-4-yl)phenyl)ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate (60 mg, 0.047 mmol, 43% yield) as a yellow oil. LCMS (ESI, m/z): 1259.5 [M]⁺.

Step 4: (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)propanamido)-3,3-dimethylbutanoyl)-N—((S)-1-(4-(3,5-difluoropyridin-4-yl)phenyl)ethyl)-4-hydroxypyrrolidine-2-carboxamide (Example 27)

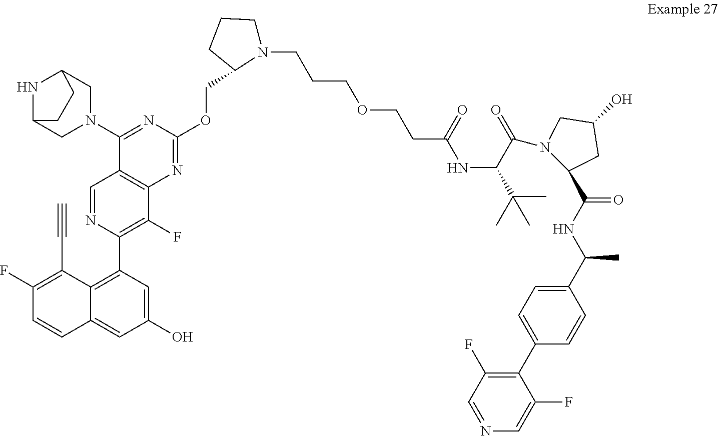 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(F)cncc6F)cc5)C(C)(C)C)nc4c3F)c12]

To a solution of tert-butyl 3-(2-(((S)-1-(3-(3-(((S)-1-((2S,4R)-2-(((S)-1-(4-(3,5-difluoropyridin-4-yl)phenyl)ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-7-(8-ethynyl-7-fluoro-3-(methoxymethoxy) naphthalen-1-yl)-8-fluoropyrido[4,3-d]pyrimidin-4-yl)-3,8-diazabicyclo[3.2.1]octane-8-carboxylate (60 mg, 0.047 mmol, 1 eq) in dichloromethane (3 mL) was added trifluoroacetic acid (1 mL). The mixture was stirred at 20° C. for 1 h. LCMS showed the desired mass was detected. The reaction mixture was filtered and concentrated under reduced pressure to give a residue. The residue was purified by prep-HPLC (column: 3_Phenomenex Luna C18 75*30 mm*3 um; mobile phase: [water(TFA)-ACN]; B %: 25%-45%, 9 min) to afford the product (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)propanamido)-3,3-dimethylbutanoyl)-N—((S)-1-(4-(3,5-difluoropyridin-4-yl)phenyl)ethyl)-4-hydroxypyrrolidine-2-carboxamide (13.4 mg, 0.01 mmol, 25% yield) as a white solid. LCMS (ESI, m/z): 1115.6 [M]⁺, ¹H NMR (400 MHz, DMSO-d₆) δ 10.09 (d, J=11.2 Hz, 1H), 9.03 (s, 1H), 8.68-8.62 (m, 2H), 8.36 (s, 1H), 7.94 (s, 1H), 7.88-7.79 (m, 1H), 7.53-7.38 (m, 6H), 7.16 (s, 1H), 5.26-5.06 (m, 1H), 4.85 (d, J=2.8, 6.4 Hz, 1H), 4.55-4.39 (m, 3H), 4.20 (s, 3H), 4.12-4.02 (m, 1H), 3.97-3.88 (m, 1H), 3.71-3.51 (m, 8H), 2.20-2.15 (m, 2H), 2.02-1.96 (m, 2H), 1.66 (b s, 7H), 1.38 (b d, J=7.2 Hz, 3H), 1.25-1.14 (m, 9H), 0.90 (b d, J=2.4 Hz, 9H).

Example 28: (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl) propoxy)propanamido)-3,3-dimethylbutanoyl)-N—((S)-1-(4-(3-cyanopyridin-4-yl)phenyl)ethyl)-4-hydroxypyrrolidine-2-carboxamide

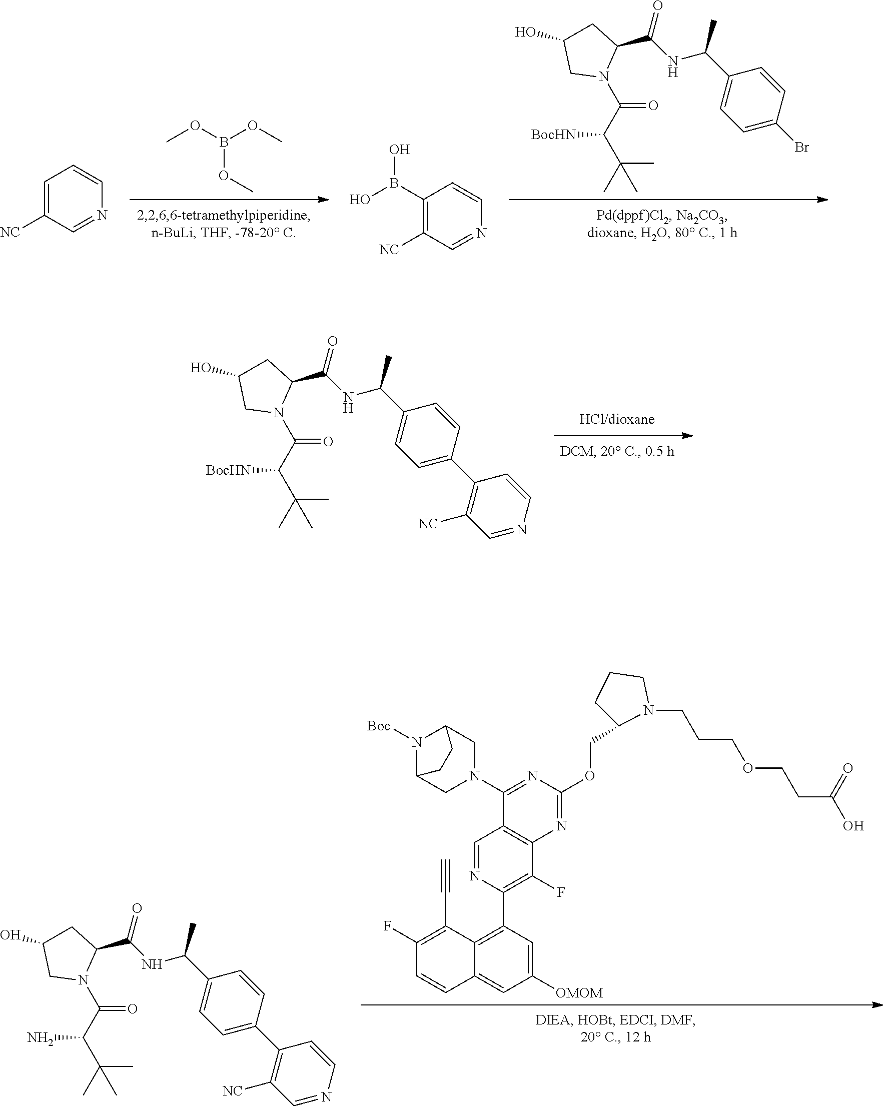

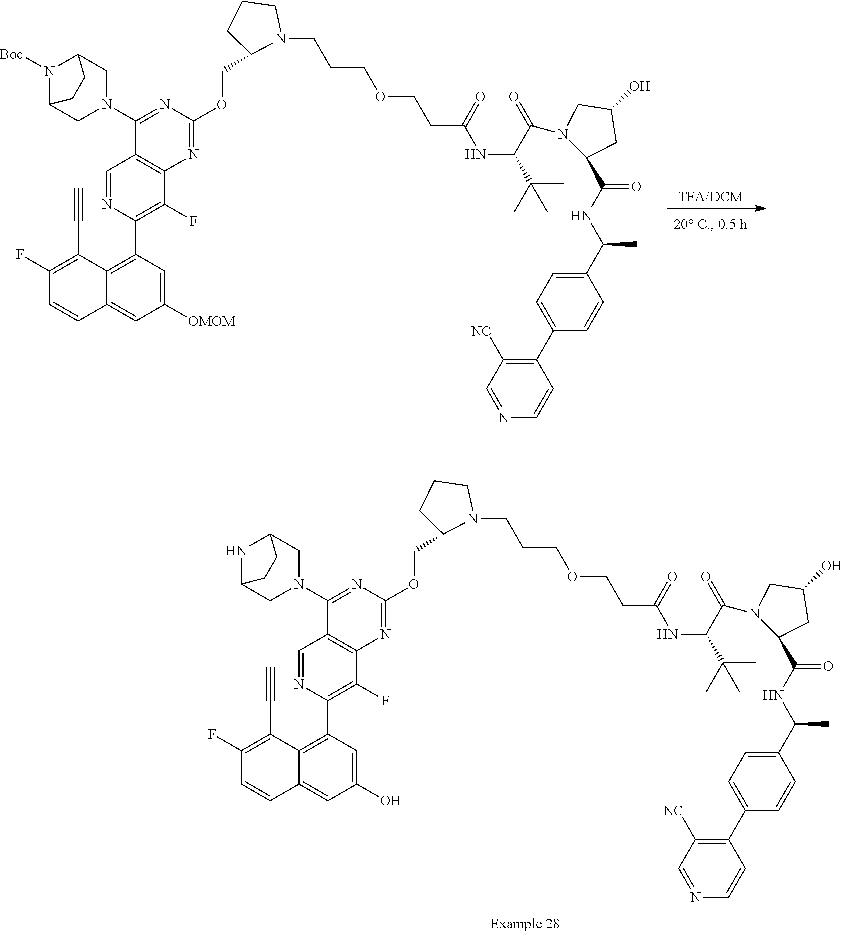

Step 1: (3-cyanopyridin-4-yl)boronic acid

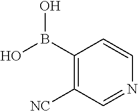 [SMILES: N#Cc1cnccc1B(O)O]

To a stirred solution of 2,2,6,6-tetramethylpiperidine (2.85 g, 20.2 mmol, 2.10 eq) in tetrahydrofuran (40 mL) was added n-butyl lithium (2.5 M, 7.7 mL, 2 eq) at −30° C. in nitrogen. The solution was allowed to warm to 0° C., and stirred at 0° C. for 15 min. Then the mixture was cooled to −78° C. before the solution of nicotinonitrile (1 g, 9.61 mmol, 1 eq) in tetrahydrofuran (20 mL) was slowly added. After 30 min, a solution of trimethyl borate (2.10 g, 20.2 mmol, 2.10 eq) in tetrahydrofuran (10 mL) was slowly added over 15 min, and the reaction mixture was stirred at −78° C. for 30 min. The solution was then allowed to warm slowly to 20° C. The reaction mixture was quenched with water (40 mL) and washed with petroleum ether (75 mL×3). The aqueous layer was then acidified to pH=6 by the addition of aqueous hydrochloride (3 M), extracted with ethyl acetate (100 mL×5), and the organic layer was evaporated under reduced pressure to afford (3-cyanopyridin-4-yl) boronic acid (400 mg, crude) as a brown solid. ¹H NMR (400 MHz, DMSO-d₆) δ 8.88 (d, J=1.2 Hz, 1H), 8.82-8.76 (m, 2H), 8.69-8.62 (m, 1H), 8.20-8.13 (m, 1H), 7.68 (d, J=5.2 Hz, 1H), 7.60-7.53 (m, 1H).

Step 2: tert-butyl ((S)-1-((2S,4R)-2-(((S)-1-(4-(3-cyanopyridin-4-yl)phenyl)ethyl)carbamoyl)-4-hydroxypyrrolidin-1-yl)-3,3-dimethyl-1-oxobutan-2-yl)carbamate

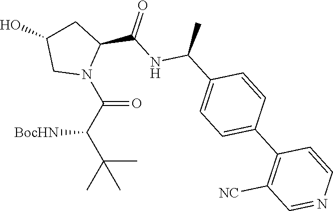 [SMILES: C[C@H](NC(=O)[C@@H]1C[C@@H](O)CN1C(=O)[C@@H](NC(=O)OC(C)(C)C)C(C)(C)C)c1ccc(-c2ccncc2C#N)cc1]

To a solution of tert-butyl ((S)-1-((2S,4R)-2-(((S)-1-(4-bromophenyl)ethyl)carbamoyl)-4-hydroxypyrrolidin-1-yl)-3,3-dimethyl-1-oxobutan-2-yl)carbamate (281 mg, 1.90 mmol, 2 eq) in dioxane (5 mL) was added sodium carbonate (433 mg, 2.85 mmol, 3 eq), ditert-butyl (cyclopentyl)phosphane; dichloropalladium; iron (62 mg, 0.095 mmol, 0.1 eq) and water (1 mL). The mixture was stirred at 80° C. for 1 h. LCMS showed the desired mass was detected. The reaction mixture was diluted with water (10 mL) and extracted with ethyl acetate (20 mL×3). The combined organic layers were washed with brine (10 mL×3), dried over anhydrous sodium sulfate, filtered and concentrated under reduced pressure to give a residue. The residue was purified by prep-TLC (dichloromethane/methanol=10/1) to afford the product tert-butyl ((S)-1-((2S,4R)-2-(((S)-1-(4-(3-cy anopyridin-4-yl)phenyl)ethyl)carbamoyl)-4-hydroxypyrrolidin-1-yl)-3,3-dimethyl-1-oxobutan-2-yl)carbamate (500 mg, 0.91 mmol, 95% yield) as a yellow solid. LCMS (ESI, m/z): 550.3 [M+1]⁺

Step 3: (2S,4R)-1-((S)-2-amino-3,3-dimethylbutanoyl)-N—((S)-1-(4-(3-cyanopyridin-4-yl)phenyl) ethyl)-4-hydroxypyrrolidine-2-carboxamide

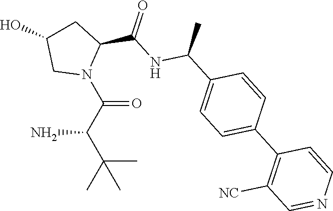 [SMILES: C[C@H](NC(=O)[C@@H]1C[C@@H](O)CN1C(=O)[C@@H](N)C(C)(C)C)c1ccc(-c2ccncc2C#N)cc1]

To a solution of tert-butyl ((S)-1-((2S,4R)-2-(((S)-1-(4-(3-cyanopyridin-4-yl)phenyl)ethyl) carbamoyl)-4-hydroxypyrrolidin-1-yl)-3,3-dimethyl-1-oxobutan-2-yl)carbamate (150 mg, 0.27 mmol, 1 eq) in dichloromethane (2 mL) was added hydrochloride/dioxane (4 M, 1 mL, 14.66 eq). The mixture was stirred at 20° C. for 0.5 h. LCMS showed the desired mass was detected. The reaction mixture was concentrated under reduced pressure to afford the crude product (2S,4R)-1-((S)-2-amino-3,3-dimethylbutanoyl)-N—((S)-1-(4-(3-cyanopyridin-4-yl)phenyl)ethyl)-4-hydroxypyrrolidine-2-carboxamide (120 mg, 0.25 mmol, 90% yield, hydrochloride) as a yellow solid, which was used into next step directly. LCMS (ESI, m/z): 450.4 [M+1]⁺

Step 4: tert-butyl 3-(2-(((S)-1-(3-(3-(((S)-1-((2S,4R)-2-(((S)-1-(4-(3-cyanopyridin-4-yl)phenyl) ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-7-(8-ethynyl-7-fluoro-3-(methoxymethoxy) naphthalen-1-yl)-8-fluoropyrido[4,3-d]pyrimidin-4-yl)-3,8-diazabicyclo[3.2.1]octane-8-carboxylate

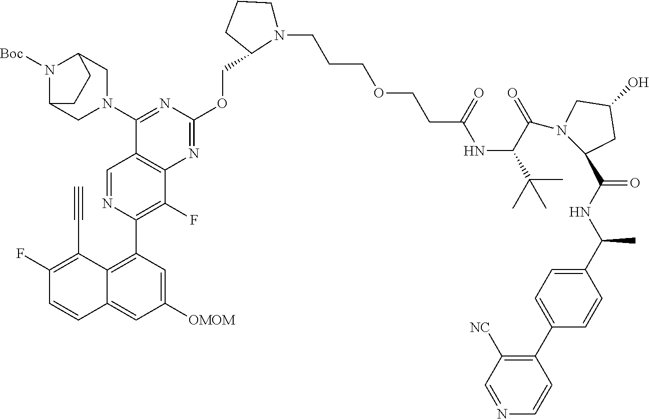

To a solution of (2S,4R)-1-((S)-2-amino-3,3-dimethylbutanoyl)-N—((S)-1-(4-(3-cyanopyridin-4-yl)phenyl)ethyl)-4-hydroxypyrrolidine-2-carboxamide (50 mg, 0.11 mmol, 1 eq) was added diisopropylethylamine (71 mg, 0.55 mmol, 5 eq), 3-(3-((2S)-2-(((4-(8-(tert-butoxycarbonyl)-3,8-diazabicyclo[3.2.1] octan-3-yl)-7-(8-ethynyl-7-fluoro-3-(methoxymethoxy)naphthalen-1-yl)-8-fluoropyrido[4,3-d]pyrimidin-2-yl)oxy)methyl)pyrrolidin-1-yl)propoxy)propanoic acid (90 mg, 0.11 mmol, 1 eq) and 1-hydroxybenzotriazole (30 mg, 0.22 mmol, 2 eq). The mixture was stirred at 20° C. for 30 mn. N-(3-dimethylaminopropyl)-N-ethylcarbodiimide hydrochloride (42 mg, 0.22 mmol, 2 eq) was added to the mixture, and the mixture was stirred at 20° C. for 11.5 h. LCMS showed the desired mass was detected. The reaction mixture was diluted with water (10 mL) and extracted with ethyl acetate (20 mL×3). The combined organic layers were washed with brine (10 mL×3), dried over anhydrous sodium sulfate, filtered and concentrated under reduced pressure to give a residue. The residue was purified by prep-TLC (dichloromethane/methanol=10/1) to afford tert-butyl 3-(2-(((S)-1-(3-(3-(((S)-1-((2S,4R)-2-(((S)-1-(4-(3-cyanopyridin-4-yl)phenyl)ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate (80 mg, 0.064 mmol, 58% yield) as a yellow solid. LCMS (ESI, m/z): 1248.6 [M]⁺.

Step 5: (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)propanamido)-3,3-dimethylbutanoyl)-N—((S)-1-(4-(3-cyanopyridin-4-yl)phenyl)ethyl)-4-hydroxypyrrolidine-2-carboxamide (Example 28)

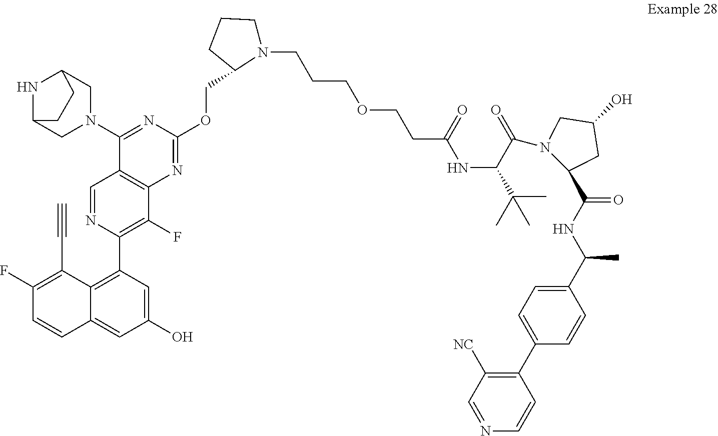 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6ccncc6C#N)cc5)C(C)(C)C)nc4c3F)c12]

To a solution of tert-butyl 3-(2-(((S)-1-(3-(3-(((S)-1-((2S,4R)-2-(((S)-1-(4-(3-cyanopyridin-4-yl) phenyl) ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-7-(8-ethynyl-7-fluoro-3-(methoxymethoxy) naphthalen-1-yl)-8-fluoropyrido[4,3-d]pyrimidin-4-yl)-3,8-diazabicyclo [3.2.1]octane-8-carboxylate (80 mg, 0.064 mmol, 1 eq) in dichloromethane (1 mL) was added trifluoroacetic acid (770 mg, 6.75 mmol, 0.5 mL, 105.38 eq). The mixture was stirred at 20° C. for 0.5 h. LCMS showed the desired mass was detected. The reaction mixture was concentrated under reduced pressure to give a residue. The residue was purified by prep-HPLC (basic condition: column: Waters X bridge 150*25 mm*5 um; mobile phase: [water (NH4HCO3)-ACN]; B %: 36%-66%, 10 min) to afford the desired product (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl) pyrrolidin-1-yl)propoxy)propanamido)-3,3-dimethylbutanoyl)-N—((S)-1-(4-(3-cyanopyridin-4-yl)phenyl)ethyl)-4-hydroxypyrrolidine-2-carboxamide (14.58 mg, 0.012 mmol, 19% yield, 91% purity) as a yellow solid. LCMS (ESI, m/z): 1104.5 [M]⁺, ¹H NMR (400 MHz, DMSO-d₆) δ 10.14 (s, 1H), 9.09 (s, 1H), 9.03 (s, 1H), 8.88 (d, J=5.2 Hz, 1H), 8.42 (d, J=8.0 Hz, 1H), 8.01-7.93 (m, 1H), 7.86-7.79 (m, 1H), 7.70 (d, J=5.2 Hz, 1H), 7.65 (d, J=8.0 Hz, 2H), 7.50-7.44 (m, 3H), 7.39 (d, J=2.4 Hz, 1H), 7.17 (d, J=2.0 Hz, 1H), 5.19-4.91 (m, 2H), 4.54-4.25 (m, 7H), 4.11-4.03 (m, 1H), 3.92 (d, J=2.4 Hz, 1H), 3.59-3.59 (m, 1H), 3.65-3.48 (m, 9H), 3.38 (d, J=2.8 Hz, 2H), 3.07-2.98 (m, 1H), 2.93-2.76 (m, 2H), 2.21-2.16 (m, 1H), 2.07-1.88 (m, 3H), 1.82-1.76 (m, 1H), 1.65 (s, 8H), 1.39 (d, J=6.8 Hz, 3H), 1.28-1.21 (m, 1H), 0.90 (d, J=2.4 Hz, 9H).

Example 29: (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)propanamido)-3,3-dimethylbutanoyl)-4-hydroxy-N—((R)-2-hydroxy-1-(4-(4-methylthiazol-5-yl)phenyl)ethyl)pyrrolidine-2-carboxamide

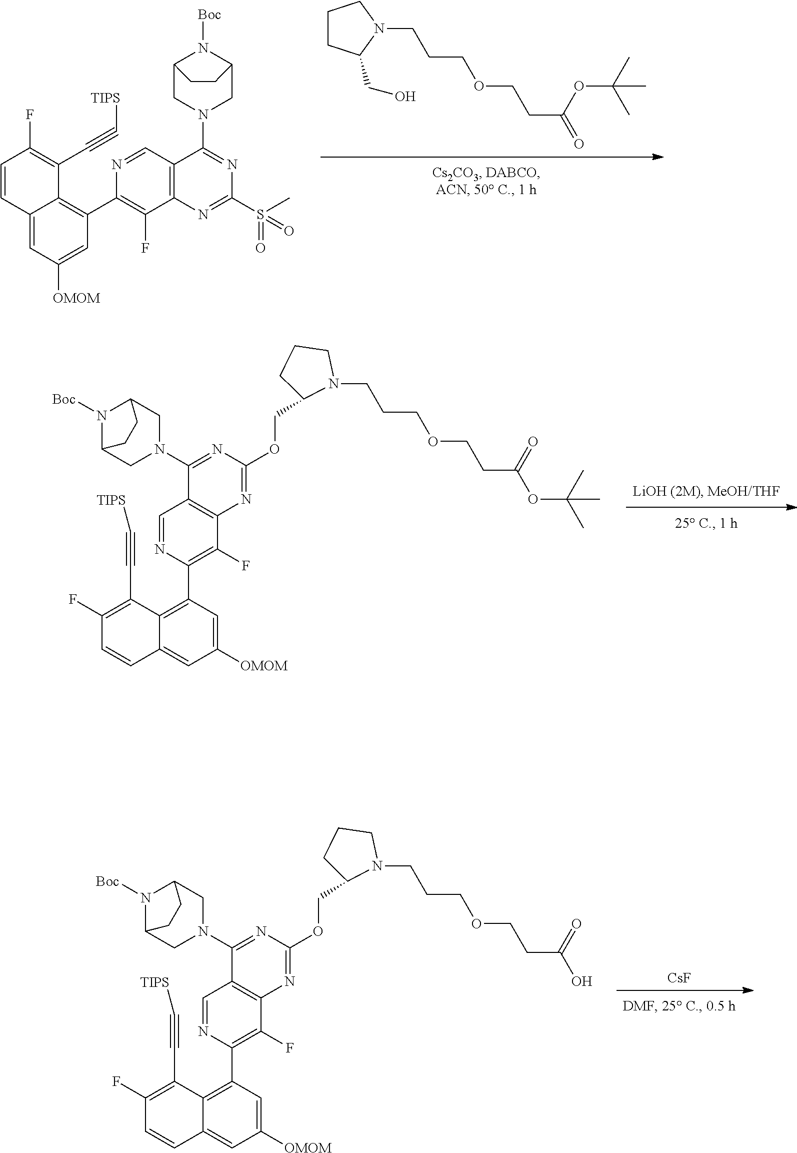

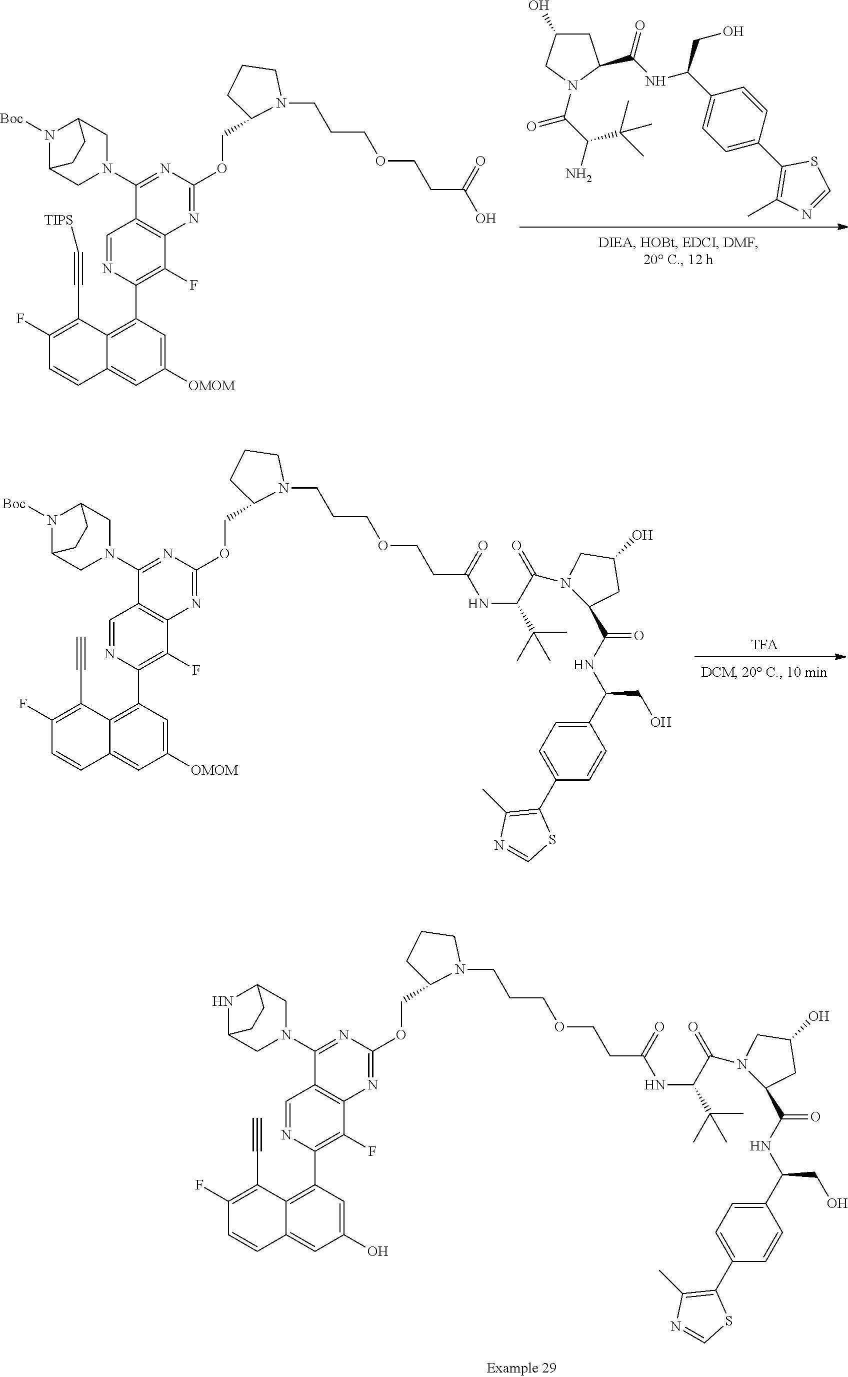

Step 1: tert-butyl 3-(2-(((S)-1-(3-(3-(tert-butoxy)-3-oxopropoxy)propyl)pyrrolidin-2-yl)methoxy)-8-fluoro-7-(7-fluoro-3-(methoxymethoxy)-8-((triisopropylsilyl)ethynyl)naphthalen-1-yl)pyrido[4,3-d]pyrimidin-4-yl)-3,8-diazabicyclo [3.2.1]octane-8-carboxylate

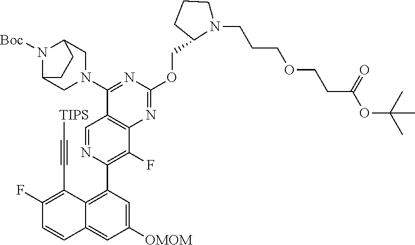

To a solution of tert-butyl (1R,5S)-3-(8-fluoro-7-(7-fluoro-3-(methoxymethoxy)-8-((triisopropylsilyl)ethynyl)naphthalen-1-yl)-2-(methylsulfonyl)pyrido[4,3-d]pyrimidin-4-yl)-3,8-diazabicyclo[3.2.1]octane-8-carboxylate, tert-butyl (S)-3-(3-(2-(hydroxymethyl)pyrrolidin-1-yl)propoxy)propanoate (1.26 g, 4.38 mmol, 1.2 eq) in acetonitrile (40 mL) was added cesium carbonate (3.57 g, 10.95 mmol, 3 eq) and 1,4-diaza-bicyclo[2.2.2]octane (40 mg, 0.36 mmol, 0.1 eq). The mixture was stirred at 50° C. for 1 h. The reaction mixture was filtered and the filtrate was concentrated in reduced pressure to give a residue. The residue was purified by prep-HPLC (column: Phenomenex Synergi Max-RP 250*50 mm*10 um; mobile phase: [water(FA)-ACN]; B %: 50%-80%, 20 min) to afford the product tert-butyl 3-(2-(((S)-1-(3-(3-(tert-butoxy)-3-oxopropoxy)propyl)pyrrolidin-2-yl)methoxy)-8-fluoro-7-(7-fluoro-3-(methoxymethoxy)-8-((triisopropylsilyl)ethynyl)naphthalen-1-yl)pyrido[4,3-d]pyrimidin-4-yl)-3,8-diazabicyclo[3.2.1]octane-8-carboxylate (2.3 g, 2.23 mmol, 61% yield) as a colorless oil. LCMS (ESI, m/z): 1029.5 [M+H]⁺.

Step 2: 3-(3-((2S)-2-(((4-(8-(tert-butoxycarbonyl)-3,8-diazabicyclo[3.2.1]octan-3-yl)-8-fluoro-7-(7-fluoro-3-(methoxymethoxy)-8-((triisopropylsilyl)ethynyl)naphthalen-1-yl)pyrido[4,3-d]pyrimidin-2-yl)oxy)methyl)pyrrolidin-1-yl)propoxy)propanoic acid

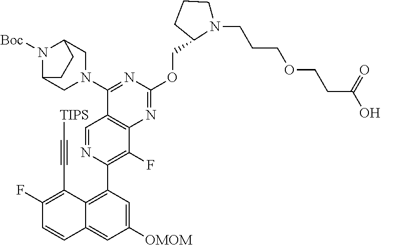

To a solution of tert-butyl 3-[2-[[(2S)-1-[3-(3-tert-butoxy-3-oxo-propoxy)propyl]pyrrolidin-2-yl]methoxy]-8-fluoro-7-[7-fluoro-3-(methoxymethoxy)-8-(2-triisopropylsilylethynyl)-1-naphthyl]pyrido[4,3-d]pyrimidin-4-yl]-3,8-diazabicyclo[3.2.1]octane-8-carboxylate (800 mg, 0.77 mmol, 1 eq) in methanol (4 mL), tetrahydrofuran (4 mL), water (2 mL) was added lithium hydroxide (326 mg, 7.77 mmol, 10 eq). The mixture was stirred at 25° C. for 1 h. The reaction mixture was adjusted pH to 5-6 by adding aqueous hydrochloric acid (1M), then the mixture was extracted with ethyl acetate (20 mL×3). The combined organic phase was washed with brine (30 mL×3), dried over anhydrous sodium sulfate, filtered and concentrated in vacuum. The residue was purified by prep-TLC (8% methanol in dichloromethane) to afford 3-(3-((2S)-2-(((4-(8-(tert-butoxycarbonyl)-3,8-diazabicyclo[3.2.1]octan-3-yl)-8-fluoro-7-(7-fluoro-3-(methoxymethoxy)-8-((triisopropylsilyl)ethynyl)naphthalen-1-yl)pyrido[4,3-d]pyrimidin-2-yl)oxy)methyl)pyrrolidin-1-yl)propoxy)propanoic acid (130 mg, 0.13 mmol, 17% yield) as a white solid. LCMS (ESI, m/z): 973.5 [M+H]⁺.

Step 3: 3-(3-((2S)-2-(((4-(8-(tert-butoxycarbonyl)-3,8-diazabicyclo[3.2.1]octan-3-yl)-7-(8-ethynyl-7-fluoro-3-(methoxymethoxy)naphthalen-1-yl)-8-fluoropyrido[4,3-d]pyrimidin-2-yl)oxy)methyl)pyrrolidin-1-yl)propoxy)propanoic acid

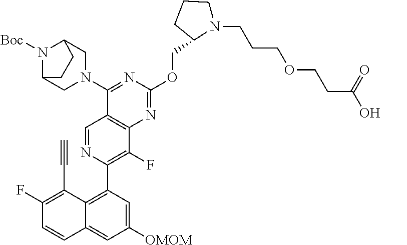

To a solution of 3-(3-((2S)-2-(((4-(8-(tert-butoxycarbonyl)-3,8-diazabicyclo[3.2.1]octan-3-yl)-8-fluoro-7-(7-fluoro-3-(methoxymethoxy)-8-((triisopropylsilyl)ethynyl)naphthalen-1-yl)pyrido[4,3-d]pyrimidin-2-yl)oxy)methyl)pyrrolidin-1-yl)propoxy)propanoic acid (130 mg, 0.13 mmol, 1 eq) in N,N-dimethylformamide (2 mL) was added cesium fluoride (304 mg, 2.00 mmol, 15 eq). The mixture was stirred at 25° C. for 0.5 h. The reaction mixture was filtered and the filtrate was extracted with ethyl acetate (20 mL×3). The combined organic phase was washed with brine (20 mL×3), dried over anhydrous sodium sulfate, filtered and the filtrate was concentrated in vacuum to get the crude product 3-(3-((2S)-2-(((4-(8-(tert-butoxycarbonyl)-3,8-diazabicyclo[3.2.1]octan-3-yl)-7-(8-ethynyl-7-fluoro-3-(methoxymethoxy)naphthalen-1-yl)-8-fluoropyrido[4,3-d]pyrimidin-2-yl)oxy)methyl)pyrrolidin-1-yl)propoxy)propanoic acid (100 mg, 0.12 mmol, 91% yield) as a yellow solid, which was used into next step without purification. LCMS (ESI, m/z): 817.4 [M+H]⁺.

Step 4: tert-butyl 3-(7-(8-ethynyl-7-fluoro-3-(methoxymethoxy)naphthalen-1-yl)-8-fluoro-2-(((S)-1-(3-(3-(((S)-1-((2S,4R)-4-hydroxy-2-(((R)-2-hydroxy-1-(4-(4-methylthiazol-5-yl)phenyl)ethyl)carbamoyl)pyrrolidin-1-yl)-3,3-dimethyl-1-oxobutan-2-yl)amino)-3-oxopropoxy)propyl)pyrrolidin-2-yl)methoxy)pyrido[4,3-d]pyrimidin-4-yl)-3,8-diazabicyclo[3.2.1]octane-8-carboxylate

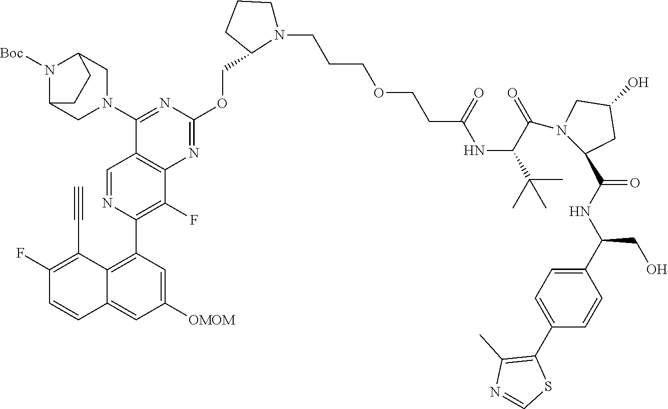

To a solution of 3-(3-((2S)-2-(((4-(8-(tert-butoxycarbonyl)-3,8-diazabicyclo[3.2.1]octan-3-yl)-7-(8-ethynyl-7-fluoro-3-(methoxymethoxy)naphthalen-1-yl)-8-fluoropyrido[4,3-d]pyrimidin-2-yl)oxy)methyl)pyrrolidin-1-yl)propoxy)propanoic acid (100 mg, 0.12 mmol, 1 eq) in dimethylformamide (2 mL) was added N,N-diisopropylethylamine (79 mg, 0.61 mmol, 5 eq) and (2S,4R)-1-((S)-2-amino-3,3-dimethylbutanoyl)-4-hydroxy-N—((R)-2-hydroxy-1-(4-(4-methylthiazol-5-yl)phenyl)ethyl)pyrrolidine-2-carboxamide (70 mg, 0.14 mmol, 1.16 eq, hydrochloride), Hydroxybenzotriazole (49 mg, 0.36 mmol, 3 eq), 1-(3-dimethylaminopropyl)-3-ethyl carbodiimide hydrochloride (70 mg, 0.36 mmol, 3 eq). The mixture was stirred at 20° C. for 12 h. Water (10 mL) was added before the reaction mixture was extracted with ethyl acetate (20 mL×3). The combined organic layers were washed with brine (30 mL×3), dried over anhydrous sodium sulfate, filtered and concentrated in vacuum to get a residue. The residue was purified by prep-TLC (10% methanol in dichloromethane) to afford the product tert-butyl 3-(7-(8-ethynyl-7-fluoro-3-(methoxymethoxy)naphthalen-1-yl)-8-fluoro-2-(((S)-1-(3-(3-(((S)-1-((2S,4R)-4-hydroxy-2-(((R)-2-hydroxy-1-(4-(4-methylthiazol-5-yl)phenyl)ethyl)carbamoyl)pyrrolidin-1-yl)-3,3-dimethyl-1-oxobutan-2-yl)amino)-3-oxopropoxy)propyl)pyrrolidin-2-yl)methoxy)pyrido[4,3-d]pyrimidin-4-yl)-3,8-diazabicyclo[3.2.1]octane-8-carboxylate (85 mg, 0.06 mmol, 55% yield) as a yellow oil. LCMS (ESI, m/z): 1259.8 [M+H]⁺

Step 5: (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)propanamido)-3,3-dimethylbutanoyl)-4-hydroxy-N—((R)-2-hydroxy-1-(4-(4-methylthiazol-5-yl)phenyl)ethyl)pyrrolidine-2-carboxamide (Example 29)

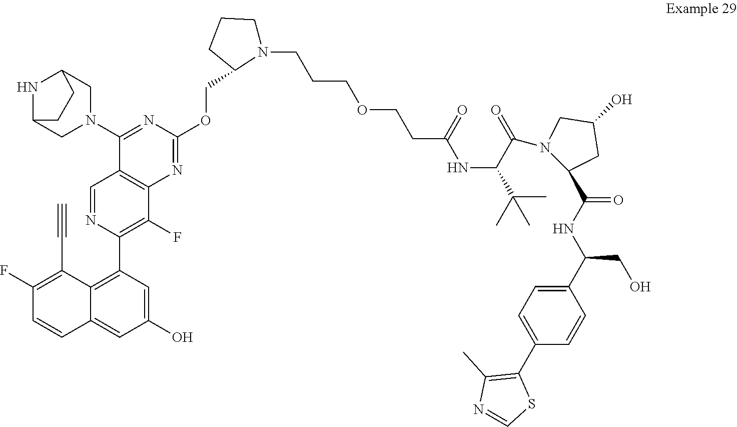 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](CO)c5ccc(-c6scnc6C)cc5)C(C)(C)C)nc4c3F)c12]

To a solution of tert-butyl 3-[7-[8-ethynyl-7-fluoro-3-(methoxymethoxy)-1-naphthyl]-8-fluoro-2-[[(2S)-1-[3-[3-[[(1S)-1-[(2S,4R)-4-hydroxy-2-[[(1R)-2-hydroxy-1-[4-(4-methylthiazol-5-yl)phenyl]ethyl]carbamoyl]pyrrolidine-1-carbonyl]-2,2-dimethyl-propyl]amino]-3-oxo-propoxy]propyl]pyrrolidin-2-yl]methoxy]pyrido[4,3-d]pyrimidin-4-yl]-3,8-diazabicyclo[3.2.1]octane-8-carboxylate (85 mg, 0.06 mmol, 1 eq) in dichloromethane (1 mL) was added trifluoroacetic acid (3.08 g, 27.01 mmol, 2 mL, 400 eq). The mixture was stirred at 20° C. for 10 min. The reaction mixture was filtered and concentrated in vacuum to give a residue. The residue was purified by prep-HPLC (column: Phenomenex luna C18 150*25 mm*10 um; mobile phase:[water(TFA)-ACN]; B^{%}: 16%-46%, 11 min) to afford the desired product (2S,4R)-1-[(2S)-2-[3-[3-[(2S)-2-[[4-(3,8-diazabicyclo [3.2.1]octan-3-yl)-7-(8-ethynyl-7-fluoro-3-hydroxy-1-naphthyl)-8-fluoro-pyrido[4,3-d] pyrimidin-2-yl]oxymethyl]pyrrolidin-1-yl]propoxy]propanoylamino]-3,3-dimethyl-butanoyl]-4-hydroxy —N-[(1R)-2-hydroxy-1-[4-(4-methylthiazol-5-yl)phenyl] ethyl]pyrrolidine-2-carboxamide (21 mg, 0.02 mmol, 28% yield, 98% purity) as a yellow solid. LCMS (ESI, m/z): 1115.4 [M+H]⁺, ¹H NMR (400 MHz, DMSO) δ 10.16 (s, 1H), 9.08 (s, 1H), 8.98 (s, 1H), 8.37-8.32 (m, 1H), 8.01-7.95 (m, 1H), 7.85-7.79 (m, 1H), 7.41 (br d, J=7.6 Hz, 6H), 7.18 (s, 1H), 4.90-4.83 (m, 1H), 4.64-4.57 (m, 1H), 4.52-4.41 (m, 3H), 4.31-4.24 (m, 1H), 4.08-3.91 (m, 3H), 3.80-3.70 (m, 2H), 3.62-3.51 (m, 6H), 3.41-3.36 (m, 2H), 3.29 (s, TOH), 2.45 (br s, 3H), 2.03-1.97 (m, 2H), 1.86 (br d, J=1.2 Hz, 2H), 1.70-1.67 (m, 2H), 1.18-1.12 (m, 4H), 0.90 (br d, J=2.4 Hz, 9H), 0.82-0.77 (m, 4H).

Example 30: (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)propanamido)-3,3-dimethylbutanoyl)-N—((S)-1-(5-(2,6-difluorophenyl)pyridin-2-yl)ethyl)-4-hydroxypyrrolidine-2-carboxamide

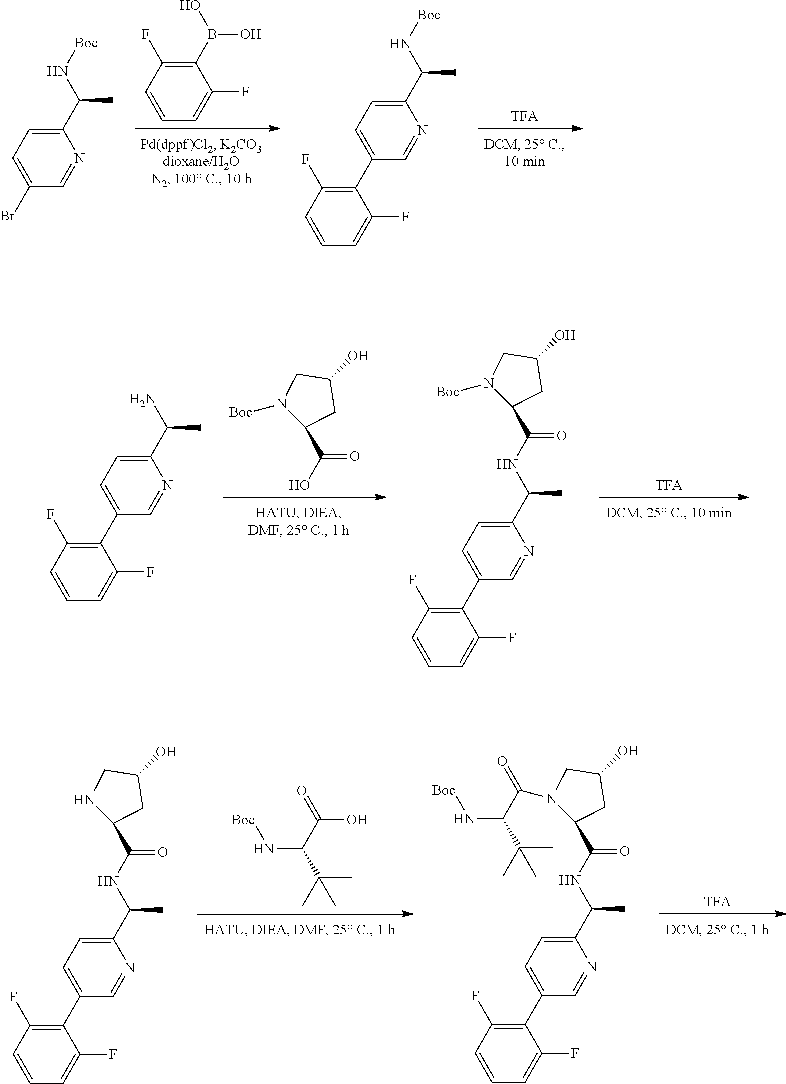

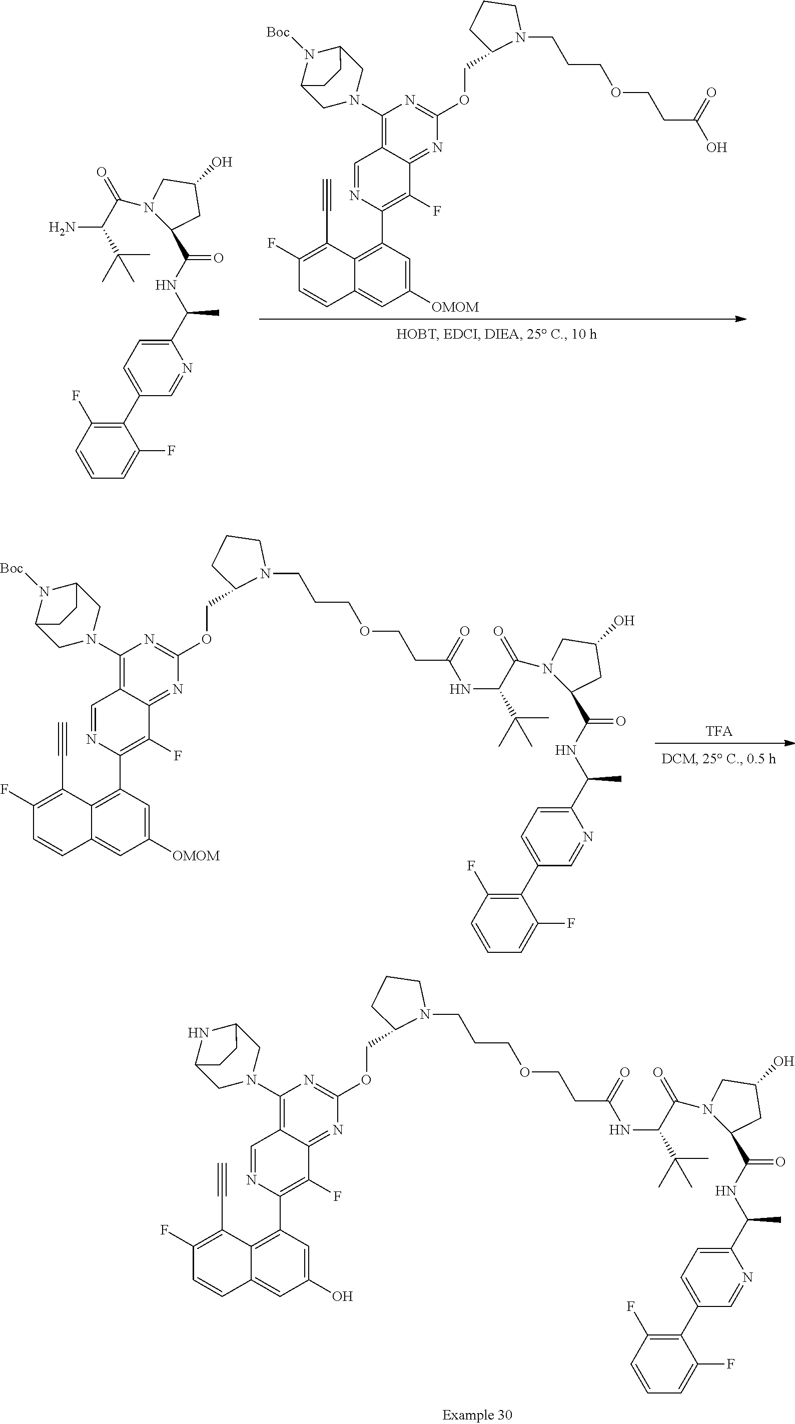

Step 1: tert-butyl (S)-(1-(5-(2,6-difluorophenyl)pyridin-2-yl)ethyl)carbamate

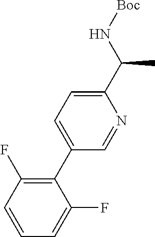 [SMILES: C[C@H](NC(=O)OC(C)(C)C)c1ccc(-c2c(F)cccc2F)cn1]

To a solution of tert-butyl N-[(1S)-1-(5-bromo-2-pyridyl)ethyl]carbamate (900 mg, 2.99 mmol) and (2,6-difluorophenyl)boronic acid (943 mg, 5.98 mmol) in water (20 mL) and dioxane (2 mL) was added [1,1-Bis(diphenylphosphino)ferrocene]dichloropalladium(II) (219 mg, 0.3 mmol) and potassium carbonate (826 mg, 5.98 mmol). The mixture was stirred at 100° C. for 12 h under nitrogen atmosphere. The reaction mixture was diluted with water (50 mL) and extracted with ethyl acetate (50 mL×3). The combined organic layers were washed with brine (50 mL), dried over anhydrous sodium sulfate and filtered. Then the filtrate was concentrated and purified by silica gel chromatography (Petroleum ether/Ethyl acetate=30/1 to 10/1) to afford the product (880 mg, 2.63 mmol, 88% yield, 100% purity) as a yellow solid. LCMS (ESI, m/z): 335.2 [M+H]⁺.

Step 2: (S)-1-(5-(2,6-difluorophenyl)pyridin-2-yl)ethan-1-amine

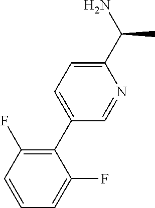 [SMILES: C[C@H](N)c1ccc(-c2c(F)cccc2F)cn1]

To a solution of tert-butyl N-[(1S)-1-(5-bromo-2-pyridyl)ethyl]carbamate (880 mg, 2.63 mmol) in dichloromethane (1 mL) was added trifluoroacetic acid (1.54 g, 13.51 mmol, 5.13 eq). The mixture was stirred at 25° C. for 10 min. The reaction mixture was concentrated under reduced pressure to get a residue, and the residue was purified by prep-HPLC (column: Phenomenex luna C18 150*40 mm*15 um; mobile phase: [water(FA)-ACN]; B %: 5%-35%, 10 min) to afford the desired product (190 mg, 0.77 mmol, 29% yield, 94% purity) as a yellow solid. LCMS (ESI, m/z): 235.1 [M+H]⁺.

Step 3: tert-butyl (2S,4R)-2-(((S)-1-(5-(2,6-difluorophenyl)pyridin-2-yl)ethyl)carbamoyl)-4-hydroxypyrrolidine-1-carboxylate

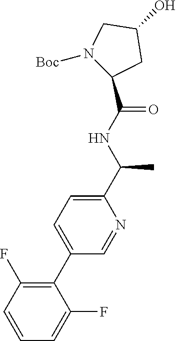 [SMILES: C[C@H](NC(=O)[C@@H]1C[C@@H](O)CN1C(=O)OC(C)(C)C)c1ccc(-c2c(F)cccc2F)cn1]

To a solution of (S)-1-(5-(2,6-difluorophenyl)pyridin-2-yl)ethan-1-amine (180 mg, 0.77 mmol) and (2S,4R)-1-tert-butoxycarbonyl-4-hydroxy-pyrrolidine-2-carboxylic acid (195 mg, 0.85 mmol) in dimethyl formamide (5 mL) was added o-(7-azabenzotriazol-1-yl)-n,n,n′,n′-tetramethyluronium hexafluorophosphate (350 mg, 0.90 mmol) and diisopropylethylamine (298 mg, 2.31 mmol). The mixture was stirred at 25° C. for 1 h. LCMS showed that the desired mass was detected. The reaction mixture was diluted with water (50 mL) and extracted with ethyl acetate (50 mL×3). The combined organic layers were washed with brine (30 mL), dried over anhydrous sodium sulfate and filtered. The filtrate was concentrated and purified by prep-HPLC (column: Phenomenex luna C18 150*40 mm*15 um; mobile phase: [water(FA)-ACN]; B %: 28%-58%, 10 min) to afford the desired product (185 mg, 0.40 mmol, 54% yield, 100% purity) as a white solid. LCMS (ESI, m/z): 448.1 [M+H]⁺.

Step 4: (2S,4R)—N—((S)-1-(5-(2,6-difluorophenyl)pyridin-2-yl)ethyl)-4-hydroxypyrrolidine-2-carboxamide

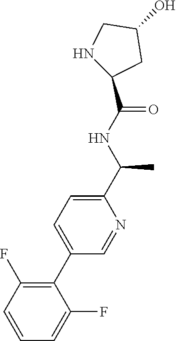 [SMILES: C[C@H](NC(=O)[C@@H]1C[C@@H](O)CN1)c1ccc(-c2c(F)cccc2F)cn1]

To a solution of tert-butyl (2S,4R)-2-(((S)-1-(5-(2,6-difluorophenyl)pyridin-2-yl)ethyl)carbamoyl)-4-hydroxypyrrolidine-1-carboxylate (180 mg, 0.40 mmol) in dichloromethane (1 mL) was added trifluoroacetic acid (1.54 g, 13.51 mmol). The mixture was stirred at 25° C. for 10 min. LCMS showed that the desired mass was detected. The reaction mixture was concentrated under reduced pressure to afford the crude product (180 mg, 0.39 mmol, 97% yield, trifluoroacetate) as a yellow oil. LCMS (ESI, m/z): 348.1 [M+H]⁺.

Step 5: tert-butyl ((S)-1-((2S,4R)-2-(((S)-1-(5-(2,6-difluorophenyl)pyridin-2-yl)ethyl)carbamoyl)-4-hydroxypyrrolidin-1-yl)-3,3-dimethyl-1-oxobutan-2-yl)carbamate

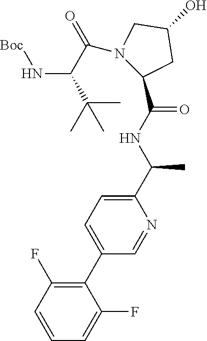 [SMILES: C[C@H](NC(=O)[C@@H]1C[C@@H](O)CN1C(=O)[C@@H](NC(=O)OC(C)(C)C)C(C)(C)C)c1ccc(-c2c(F)cccc2F)cn1]

To a solution of (2S,4R)—N—((S)-1-(5-(2,6-difluorophenyl)pyridin-2-yl)ethyl)-4-hydroxypyrrolidine-2-carboxamide (180 mg, 0.39 mmol, trifluoroacetate) and (2S)-2-(tert-butoxycarbonylamino)-3,3-dimethyl-butanoic acid (108 mg, 0.47 mmol) in N,N-dimethyl formamide (3 mL) was added o-(7-azabenzotriazol-1-yl)-n,n,n′,n′-tetramethyluronium hexafluorophosphate (193 mg, 0.50 mmol) and diisopropylethylamine (252.11 mg, 1.95 mmol). The mixture was stirred at 25° C. for 1 h. The reaction mixture was filtered, then the filtrate was concentrated and purified by prep-HPLC (column: Phenomenex luna C18 150*40 mm*15 um; mobile phase: [water(FA)-ACN]; B %: 39%-69%, 10 min) to afford the desired product (190 mg, 0.34 mmol, 87% yield, 100% purity) as a white solid. LCMS (ESI, m/z): 561.2 [M+H]⁺.

Step 6: (2S,4R)-1-((S)-2-amino-3,3-dimethylbutanoyl)-N—((S)-1-(5-(2,6-difluorophenyl)pyridin-2-yl)ethyl)-4-hydroxypyrrolidine-2-carboxamide

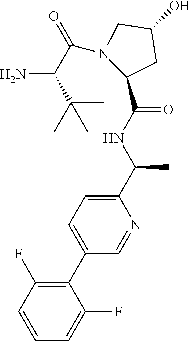 [SMILES: C[C@H](NC(=O)[C@@H]1C[C@@H](O)CN1C(=O)[C@@H](N)C(C)(C)C)c1ccc(-c2c(F)cccc2F)cn1]

To a solution of tert-butyl ((S)-1-((2S,4R)-2-(((S)-1-(5-(2,6-difluorophenyl)pyridin-2-yl)ethyl)carbamoyl)-4-hydroxypyrrolidin-1-yl)-3,3-dimethyl-1-oxobutan-2-yl)carbamate (70 mg, 0.12 mmol) in dichloromethane (1 mL) was added trifluoroacetic acid (1.54 g, 13.51 mmol). The mixture was stirred at 25° C. for 1 h. The reaction mixture was concentrated under reduced pressure to afford the crude product (70 mg, 0.12 mmol, 97% yield, trifluoroacetate) as a yellow oil, which was used into next step directly. LCMS (ESI, m/z): 461.2 [M+H]⁺.

Step 7: tert-butyl 3-(2-(((S)-1-(3-(3-(((S)-1-((2S,4R)-2-(((S)-1-(5-(2,6-difluorophenyl)pyridin-2-yl) ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate

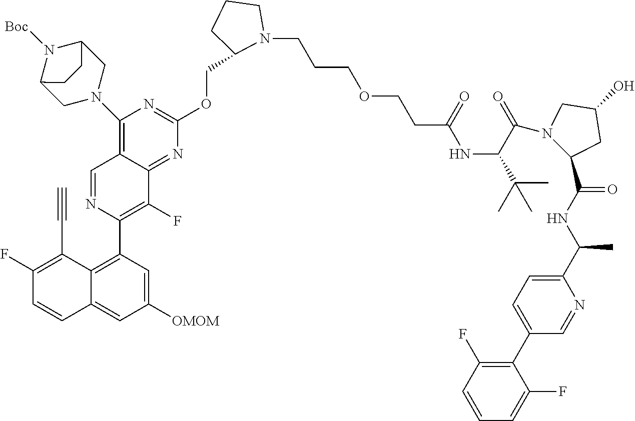

To a solution of 3-[3-[(2S)-2-[[4-(8-tert-butoxycarbonyl-3,8-diazabicyclo[3.2.1]octan-3-yl)-7-[8-ethynyl-7-fluoro-3-(methoxymethoxy)-1-naphthyl]-8-fluoro-pyrido[4,3-d]pyrimidin-2-yl]oxymethyl]pyrrolidin-1-yl]propoxy]propanoic acid (90 mg, 0.11 mmol) and(2S,4R)-1-((S)-2-amino-3,3-dimethylbutanoyl)-N—((S)-1-(5-(2,6-difluorophenyl)pyridin-2-yl)ethyl)-4-hydroxypyrrolidine-2-carboxamide (51 mg, 0.88 mmol, trifluoroacetate) in N,N-dimethyl formamide (5 mL) was added 1-hydroxybenzotriazole (22 mg, 0.16 mmol), 1-(3-Dimethylaminopropyl)-3-ethylcarbodiimide Hydrochloride (32 mg, 0.16 mmol) and diisopropylethylamine (43 mg, 0.33 mmol). The mixture was stirred at 25° C. for 10 h. The reaction mixture was filtered, then the filtrate was concentrated and purified by prep-HPLC (column: Phenomenex Synergi Polar-RP 100*25 mm*4 um; mobile phase: [water(TFA)-ACN]; B %: 48%-68%, 7 min) to afford the product (60 mg, 0.05 mmol, 43% yield) as a white solid. LCMS (ESI, m/z): 1259.6 [M]⁺.

Step 8: (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)propanamido)-3,3-dimethylbutanoyl)-N—((S)-1-(5-(2,6-difluorophenyl)pyridin-2-yl)ethyl)-4-hydroxypyrrolidine-2-carboxamide (Example 30)

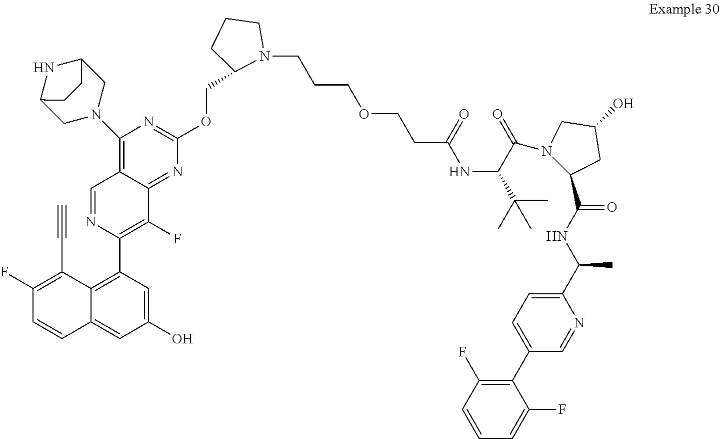 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(F)cccc6F)cn5)C(C)(C)C)nc4c3F)c12]

To a solution of tert-butyl 3-(2-(((S)-1-(3-(3-(((S)-1-((2S,4R)-2-(((S)-1-(5-(2,6-difluorophenyl)pyridin-2-yl)ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate (50 mg, 0.04 mmol) in dichloromethane (3 mL) was added trifluoroacetic acid (1 mL). The mixture was stirred at 25° C. for 0.5 h. LCMS showed that the desired mass was detected. The reaction mixture was diluted with water (50 mL) and extracted with ethyl acetate (50 mL×3). The combined organic layers were washed with brine (50 mL), dried over anhydrous sodium sulfate and filtered, then the filtrate was concentrated and purified by prep-HPLC (column: Unisil 3-100 C18 Ultra 150*50 mm*3 um; mobile phase: [water(FA)-ACN]; B %: 10%-40%, 7 min) to afford the product (6.1 mg, 13% yield, 95% purity) as a yellow solid. LCMS (ESI, m/z): 1115.4 [M]⁺, ¹H NMR (400 MHz, DMSO) δ 10.29-10.08 (m, 1H), 9.03 (s, 1H), 8.58 (s, 1H), 8.40 (d, J=7.6 Hz, 1H), 7.96 (dd, J=6.0, 8.8 Hz, 1H), 7.85 (m, 2H), 7.56-7.50 (m, 1H), 7.48-7.42 (m, 2H), 7.39 (d, J=2.4 Hz, 1H), 7.30-7.22 (m, 3H), 7.18 (d, J=2.0 Hz, 1H), 4.95 (m, 1H), 4.53-4.44 (m, 3H), 4.40-4.25 (m, 4H), 4.13-4.02 (m, 1H), 3.92 (d, J=2.8 Hz, 1H), 3.68-3.48 (m, 10H), 3.06-3.02 (m, 1H), 2.89-2.76 (m, 2H), 2.20-2.12 (m, 2H), 2.04-1.97 (m, 2H), 1.94-1.82 (m, 4H), 1.71-1.60 (m, 10H), 1.42 (d, J=6.8 Hz, 3H), 1.23 (s, 6H), 0.90 (d, J=1.6 Hz, 13H).

Example 31: (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)propanamido)-3,3-dimethylbutanoyl)-N—((S)-1-(6-(2,6-difluorophenyl)pyridin-3-yl)ethyl)-4-hydroxypyrrolidine-2-carboxamide

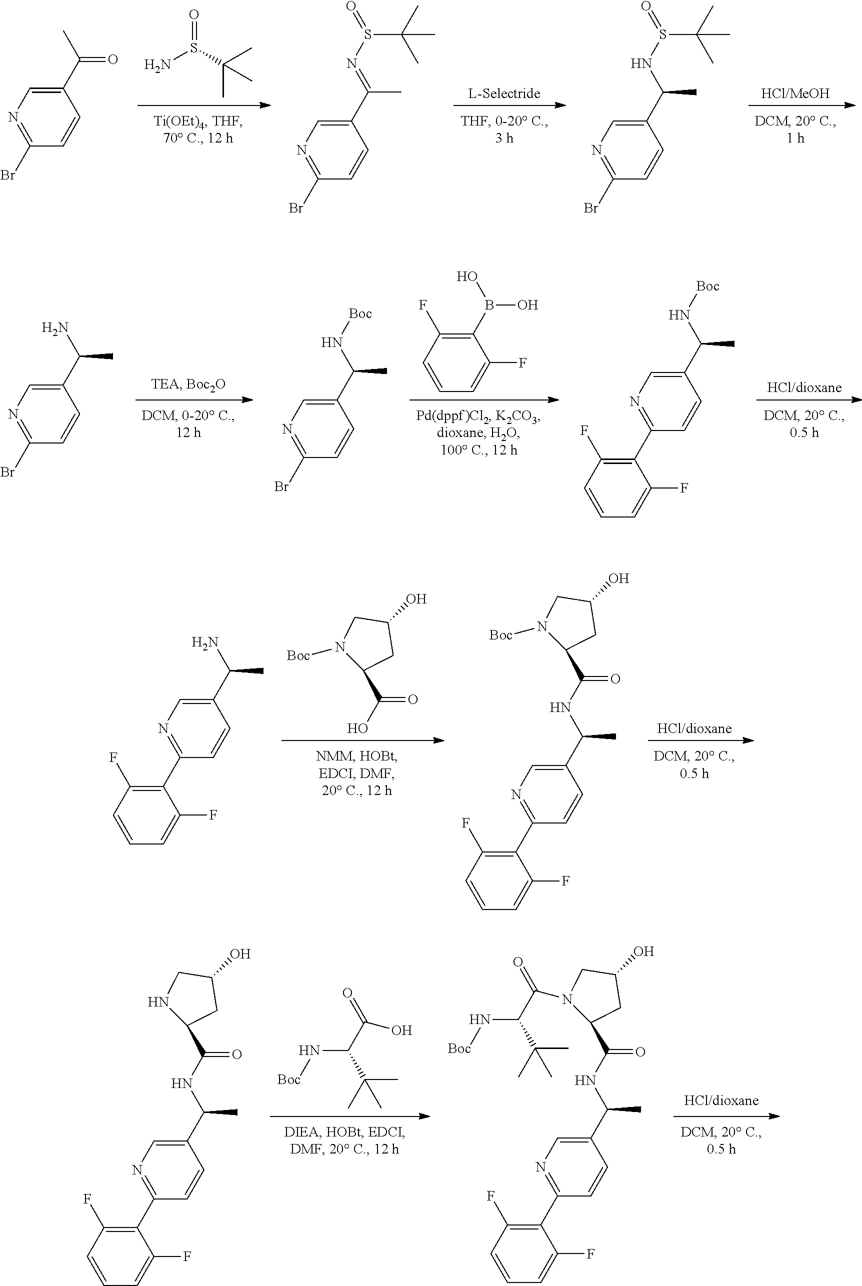

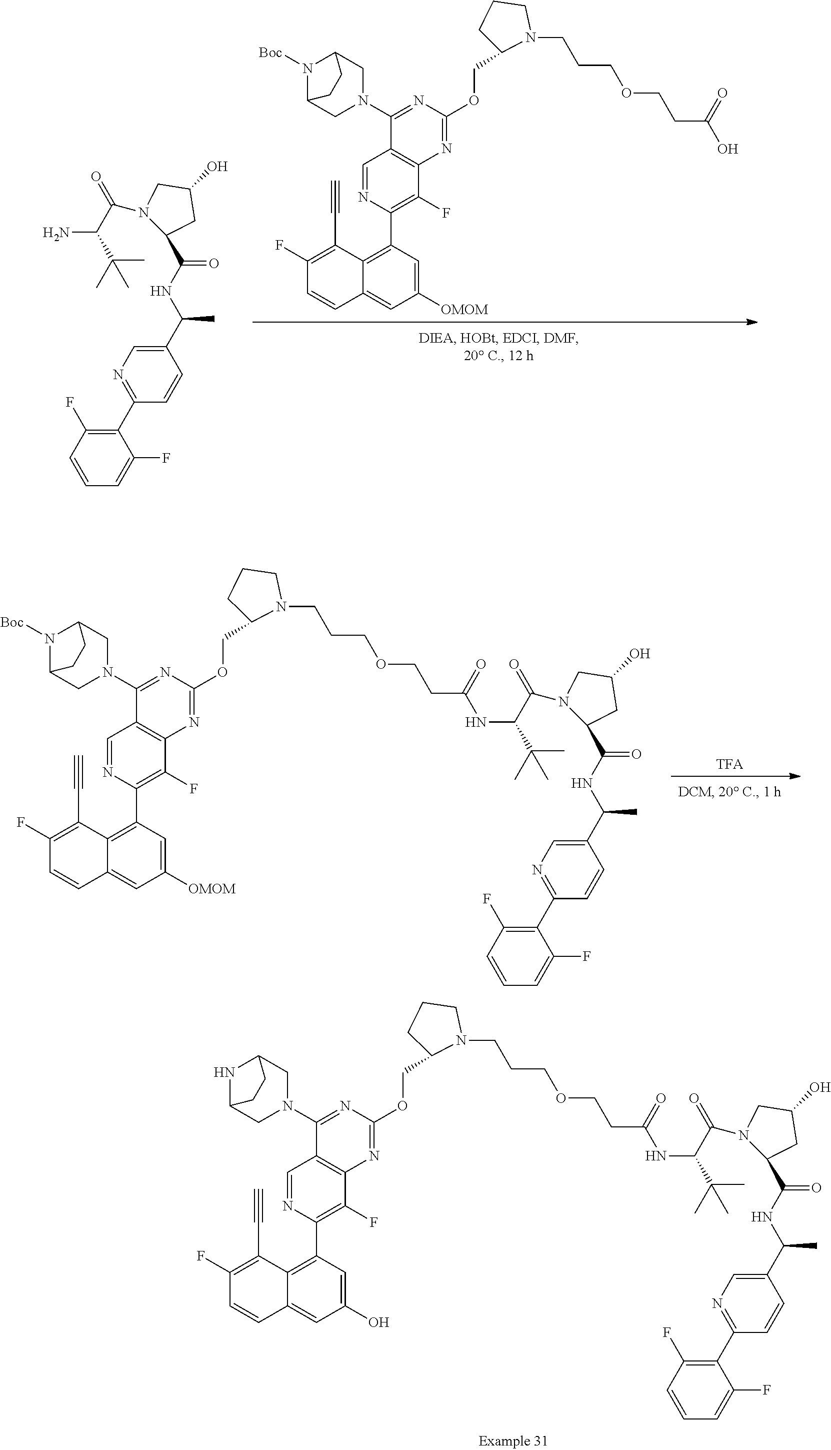

Step 1: (R,E)-N-(1-(6-bromopyridin-3-yl)ethylidene)-2-methylpropane-2-sulfinamide

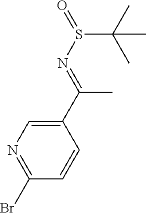 [SMILES: C/C(=N\S(=O)C(C)(C)C)c1ccc(Br)nc1]

To a solution of 1-(6-bromo-3-pyridyl)ethanone (12.00 g, 59.40 mmol, 1.20 eq) in tetrahydrofuran (60 mL) were added tetraethyl titanate (23.00 g, 99.00 mmol, 21 mL, 2.00 eq) and (R)-2-methylpropane-2-sulfinamide (6.00 g, 49.50 mmol, 1.00 eq) at 20° C. in nitrogen, and the mixture was warmed to 70° C. The mixture was stirred at 70° C. for 12 h. The reaction mixture was quenched with water (100 mL) and then the mixture was filtered. The filtrate was extracted with ethyl acetate (500 mL×3) and the combined organic layers were washed with water (500 mL), brine (500 mL), dried over sodium sulfate and then concentrated under reduced pressure to give a residue. The residue was purified by column chromatography on silica gel (petroleum ether/ethyl acetate=10/1 to 1/1) to give the product (R,E)-N-(1-(6-bromopyridin-3-yl)ethylidene)-2-methylpropane-2-sulfinamide (13 g, 42.87 mmol, 87% yield) as a green oil. ¹H NMR (400 MHz, DMSO-d₆) δ 8.84 (s, 1H), 8.24-8.09 (m, 1H), 7.77 (d, J=8.4 Hz, 1H), 2.74 (s, 3H), 1.22 (s, 9H).

Step 2: (R)—N—((S)-1-(6-bromopyridin-3-yl)ethyl)-2-methylpropane-2-sulfinamide

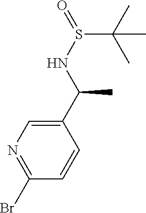 [SMILES: C[C@H](NS(=O)C(C)(C)C)c1ccc(Br)nc1]

To a solution of (R,E)-N-(1-(6-bromopyridin-3-yl)ethylidene)-2-methylpropane-2-sulfinamide (7.00 g, 23.09 mmol, 1.00 eq) in tetrahydrofuran (100 mL) was added L-selectride (1 M, 70 mL, 3.03 eq) at 0° C. under nitrogen and the mixture was warmed to 20° C. The mixture was stirred at 20° C. for 3 h. The reaction mixture was quenched with saturated aqueous ammonium chloride (200 mL) and extracted with ethyl acetate (100 mL×3). The combined mixture was washed with water (200 mL), brine (200 mL), dried over sodium sulfate and then concentrated under reduced pressure to give a residue. The residue was purified by preparative high liquid chromatography (column: Phenomenex luna C18 (250*70 mm, 10 um); mobile phase: [water(FA)-ACN]; B %: 25%-55%, 20 min) to give (R)—N—((S)-1-(6-bromopyridin-3-yl)ethyl)-2-methylpropane-2-sulfinamid (2.00 g, 6.55 mmol, 28% yield) as a light brown solid. LCMS (ESI, m/z): 305.0, 307.0 [M+H]⁺, ¹H NMR (400 MHz, DMSO-d₆) δ: 8.38 (d, J=2.4 Hz, 1H), 7.75 (dd, J=2.4, 8.4 Hz, 1H), 7.63 (d, J=8.4 Hz, 1H), 5.56 (d, J=5.6 Hz, 1H), 4.57-4.38 (m, 1H), 1.48 (d, J=6.8 Hz, 3H), 1.11 (s, 9H).

Step 3: (S)-1-(6-bromopyridin-3-yl)ethan-1-amine

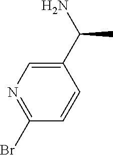

To a solution of (R)—N—((S)-1-(6-bromopyridin-3-yl)ethyl)-2-methylpropane-2-sulfinamid (5.00 g, 16.38 mmol, 1.00 eq) in dichloromethane (50 mL) was added hydrochloric acid/methanol (4 M, 50 mL, 12.21 eq). The mixture was stirred at 20° C. for 1 h. The mixture was concentrated under reduced pressure to give the crude product (S)-1-(6-bromopyridin-3-yl)ethan-1-amine (5.0 g, crude). ¹H NMR (400 MHz, DMSO-d₆) δ 8.83 (s, 3H), 8.55 (d, J=2.4 Hz, 1H), 8.02-7.98 (m, 1H), 7.74 (d, J=8.4 Hz, 1H), 1.82-1.68 (m, 1H), 1.54 (d, J=6.8 Hz, 3H).

Step 4: tert-butyl (S)-(1-(6-bromopyridin-3-yl)ethyl)carbamate

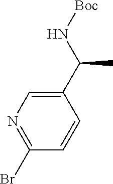 [SMILES: C[C@H](NC(=O)OC(C)(C)C)c1ccc(Br)nc1]

To a solution of (S)-1-(6-bromopyridin-3-yl)ethan-1-amine (5.00 g, 21.05 mmol, 1.00 eq, hydrochloride) in tetrahydrofuran (60 mL) was added triethylamine (10.65 g, 105.25 mmol, 15 mL, 5.00 eq) and di-tert-butyl dicarbonate (6.89 g, 31.58 mmol, 7 mL, 1.50 eq) at 0° C. and the mixture was warmed to 20° C. The mixture was stirred at 20° C. for 12 h. The mixture was diluted with water (200 mL) and extracted with ethyl acetate (100 mL×3). The combined organic layers were washed with brine (200 mL), dried over anhydrous sodium sulfate, filtered and concentrated under reduced pressure to give a residue. The residue was purified by silica gel chromatography (petroleum ether/ethyl acetate=10/1 to 5/1) to get the product tert-butyl (S)-(1-(6-bromopyridin-3-yl)ethyl)carbamate (3.60 g, 11.95 mmol, 57% yield) as a white solid. ¹H NMR: (400 MHz, DMSO-d₆) δ 8.31 (d, J=2.0 Hz, 1H), 7.70-7.65 (m, 1H), 7.64-7.58 (m, 1H), 7.55-7.43 (m, 1H), 4.69-4.55 (m, 1H), 1.36 (s, 9H), 1.31 (s, 3H)

Step 5: tert-butyl (S)-(1-(6-(2,6-difluorophenyl)pyridin-3-yl)ethyl)carbamate

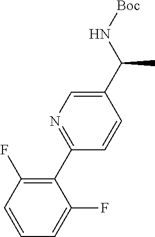 [SMILES: C[C@H](NC(=O)OC(C)(C)C)c1ccc(-c2c(F)cccc2F)nc1]

A mixture of tert-butyl (S)-(1-(6-bromopyridin-3-yl)ethyl)carbamate (500 mg, 1.66 mmol, 1 0.00 eq), (2,6-difluorophenyl)boronic acid (393 mg, 2.49 mmol, 1.50 eq), [1,1′-bis(diphenylphosphino)ferrocene]dichloropalladium(II) (243 mg, 0.33 mmol, 0.20 eq), potassium carbonate (574 mg, 4.15 mmol, 2.50 eq) in the mixed solvent of dioxane (10 mL) and water (2 mL) was degassed and purged with nitrogen for 3 times, then the mixture was stirred at 100° C. for 12 h under nitrogen atmosphere. The mixture was diluted with water (50 mL) and extracted with ethyl acetate (25 mL×3). The combined organic layers were washed with brine (50 mL), dried over anhydrous sodium sulfate, filtered and concentrated under reduced pressure to give a residue. The residue was purified by silica gel chromatography (petroleum ether/ethyl acetate=1/0 to 10/1) to get the desired product tert-butyl (S)-(1-(6-(2,6-difluorophenyl)pyridin-3-yl)ethyl)carbamate (200 mg, 0.60 mmol, 36% yield) as a white solid. LCMS (ESI, m/z): 335.1 [M+H]⁺, ¹H NMR: (400 MHz, DMSO-d₆) δ 8.63 (d, J=1.6 Hz, 1H), 7.89-7.73 (m, 1H), 7.61-7.41 (m, 3H), 7.31-7.13 (m, 2H), 4.86-4.57 (m, 1H), 1.41-1.34 (m, 12H).

Step 6: (S)-1-(6-(2,6-difluorophenyl)pyridin-3-yl)ethan-1-amine

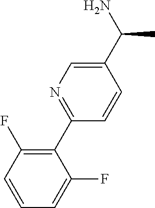 [SMILES: C[C@H](N)c1ccc(-c2c(F)cccc2F)nc1]

To a solution of tert-butyl (S)-(1-(6-(2,6-difluorophenyl)pyridin-3-yl)ethyl)carbamate (800 mg, 2.39 mmol, 1.00 eq) in dichloromethane (10 mL) was added hydrochloric acid/dioxane (4 M, 10 mL, 16.72 eq). The mixture was stirred at 20° C. for 0.5 h. The mixture was concentrated under reduced to give the crude product (S)-1-(6-(2,6-difluorophenyl)pyridin-3-yl)ethan-1-amine (640 mg, 2.36 mmol, 99% yield, hydrochloride) as a white solid, which was used into next step directly.

Step 7: tert-butyl (2S,4R)-2-(((S)-1-(6-(2,6-difluorophenyl)pyridin-3-yl)ethyl)carbamoyl)-4-hydroxypyrrolidine-1-carboxylate

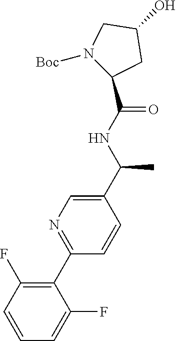 [SMILES: C[C@H](NC(=O)[C@@H]1C[C@@H](O)CN1C(=O)OC(C)(C)C)c1ccc(-c2c(F)cccc2F)nc1]

To a solution of (2S,4R)-1-tert-butoxycarbonyl-4-hydroxy-pyrrolidine-2-carboxylic acid (586 mg, 2.54 mmol, 1.05 eq) in N,N-dimethylformamide (10 mL) was added N-monomethyl morpholine (1.22 g, 12.07 mmol, 1.0 mL, 5.00 eq), N-(3-dimethylaminopropyl)-N-ethylcarbodiimide hydrochloride (695 mg, 3.62 mmol, 1.50 eq), 1-hydroxybenzotriazole (490 mg, 3.62 mmol, 1.50 eq) and (S)-1-(6-(2,6-difluorophenyl)pyridin-3-yl)ethan-1-amine (640 mg, 2.36 mmol, 1.00 eq, hydrochloride). Then the mixture was stirred at 20° C. for 12 h. The mixture was diluted with water (50 mL) and extracted with ethyl acetate (25 mL×3). The combined organic layers were washed with brine (50 ml), dried over anhydrous sodium sulfate, filtered and concentrated under reduced pressure to give a residue. The residue was purified by silica gel chromatography (petroleum ether/ethyl acetate=1/0 to 10/1) to give tert-butyl (2S,4R)-2-(((S)-1-(6-(2,6-difluorophenyl)pyridin-3-yl)ethyl)carbamoyl)-4-hydroxypyrrolidine-1-carboxylate (800 mg, 1.79 mmol, 76% yield) as a white solid. LCMS (ESI,m/z): 448.3 [M+H]⁺.

Step 8: (2S,4R)—N—((S)-1-(6-(2,6-difluorophenyl)pyridin-3-yl)ethyl)-4-hydroxypyrrolidine-2-carboxamide

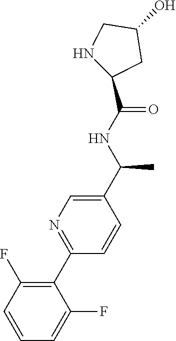 [SMILES: C[C@H](NC(=O)[C@@H]1C[C@@H](O)CN1)c1ccc(-c2c(F)cccc2F)nc1]

To a solution of tert-butyl (2S,4R)-2-(((S)-1-(6-(2,6-difluorophenyl)pyridin-3-yl)ethyl)carbamoyl)-4-hydroxypyrrolidine-1-carboxylate (800 mg, 1.79 mmol, 1.00 eq) in dichloromethane (10 mL) was added hydrochloric acid/dioxane (4 M, 6 mL, 13.42 eq). The mixture was stirred at 20° C. for 0.5 h. The mixture was stirred at 20° C. for 1 h. The reaction mixture was concentrated under reduced pressure to give the crude product (2S,4R)—N—((S)-1-(6-(2,6-difluorophenyl)pyridin-3-yl)ethyl)-4-hydroxypyrrolidine-2-carboxamide (650 mg, 1.69 mmol, 95% yield, hydrochloride) as a white solid, which was used into next step without further purification. LCMS (ESI, m/z): 348.2 [M+H]⁺.

Step 9: tert-butyl ((S)-1-((2S,4R)-2-(((S)-1-(6-(2,6-difluorophenyl)pyridin-3-yl)ethyl)carbamoyl)-4-hydroxypyrrolidin-1-yl)-3,3-dimethyl-1-oxobutan-2-yl)carbamate

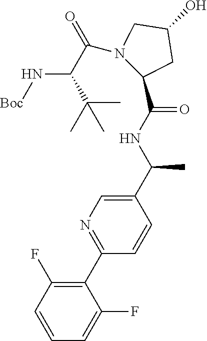 [SMILES: C[C@H](NC(=O)[C@@H]1C[C@@H](O)CN1C(=O)[C@@H](NC(=O)OC(C)(C)C)C(C)(C)C)c1ccc(-c2c(F)cccc2F)nc1]

To a solution of (2S,4R)—N—((S)-1-(6-(2,6-difluorophenyl)pyridin-3-yl)ethyl)-4-hydroxypyrrolidine-2-carboxamide (650 mg, 1.69 mmol, 1.00 eq, hydrochloride) in N,N-dimethylformamide (10 mL) was added hydrochloric acid (1.09 g, 8.47 mmol, 1 mL, 5.00 eq), N-(3-dimethylaminopropyl)-N-ethylcarbodiimide hydrochloride (490 mg, 2.54 mmol, 1.50 eq) and 1-hydroxybenzotriazole (343 mg, 2.54 mmol, 1.50 eq) and (2S)-2-(tert-butoxycarbonylamino)-3,3-dimethyl-butanoic acid (430 mg, 1.86 mmol, 1.10 eq). The mixture was stirred at 20° C. for 12 h. The mixture was diluted with water (100 mL) and extracted with ethyl acetate (50 mL×3). The combined organic layers were washed with brine (100 mL), dried over anhydrous sodium sulfate filtered and concentrated under reduced pressure to give a residue. The residue was purified by silica gel chromatography (petroleum ether/ethyl acetate=1/0 to 10/1) to give tert-butyl ((S)-1-((2S,4R)-2-(((S)-1-(6-(2,6-difluorophenyl)pyridin-3-yl)ethyl)carbamoyl)-4-hydroxypyrrolidin-1-yl)-3,3-dimethyl-1-oxobutan-2-yl)carbamate (590 mg, 1.03 mmol, 61% yield, 98% purity) as a white solid. LCMS (ESI, m/z): 561.3 [M+H]⁺.

Step 10:_(2S,4R)-1-((S)-2-amino-3,3-dimethylbutanoyl)-N—((S)-1-(6-(2,6-difluorophenyl)pyridin-3-yl)ethyl)-4-hydroxypyrrolidine-2-carboxamide

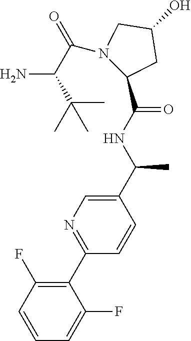 [SMILES: C[C@H](NC(=O)[C@@H]1C[C@@H](O)CN1C(=O)[C@@H](N)C(C)(C)C)c1ccc(-c2c(F)cccc2F)nc1]

To a solution of tert-butyl ((S)-1-((2S,4R)-2-(((S)-1-(6-(2,6-difluorophenyl)pyridin-3-yl)ethyl)carbamoyl)-4-hydroxypyrrolidin-1-yl)-3,3-dimethyl-1-oxobutan-2-yl)carbamate (590 mg, 1.05 mmol, 1.00 eq) in dichloromethane (12 mL) was added hydrochloric acid/dioxane (4 M, 5 mL, 1.00 eq). The mixture was stirred at 20° C. for 0.5 h. The mixture was concentrated under reduced pressure to give the crude product (2S,4R)-1-((S)-2-amino-3,3-dimethylbutanoyl)-N—((S)-1-(6-(2,6-difluorophenyl)pyridin-3-yl)ethyl)-4-hydroxypyrrolidine-2-carboxamide (500 mg, 1.01 mmol, 96% yield, hydrochloride) as a white solid, which was used directly into next step. LCMS (ESI, m/z): 461.2 [M+H]⁺.

Step 11: tert-butyl 3-(2-(((S)-1-(3-(3-(((S)-1-((2S,4R)-2-(((S)-1-(6-(2,6-difluorophenyl)pyridin-3-yl)ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo [3.2.1]octane-8-carboxylate

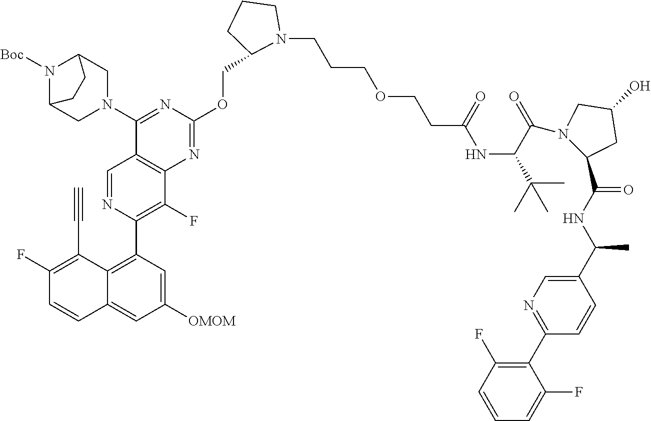

To a solution of 3-[3-[(2S)-2-[[4-(8-tert-butoxycarbonyl-3,8-diazabicyclo[3.2.1]octan-3-yl)-7-[8-ethynyl-7-fluoro-3-(methoxymethoxy)-1-naphthyl]-8-fluoro-pyrido[4,3-d]pyrimidin-2-yl]oxymethyl]pyrrolidin-1-yl]propoxy]propanoic acid (127 mg, 0.15 mmol, 1.00 eq) in N,N-dimethylformamide (3 mL) was added 1-hydroxybenzotriazole (32 mg, 0.23 mmol, 1.50 eq), N-(3-dimethylaminopropyl)-N-ethylcarbodiimide hydrochloride (44 mg, 0.23 mmol, 1.50 eq) and diisopropylethylamine (148 mg, 1.15 mmol, 7.39 eq). Then (2S,4R)-1-((S)-2-amino-3,3-dimethylbutanoyl)-N—((S)-1-(6-(2,6-difluorophenyl)pyridin-3-yl)ethyl)-4-hydroxypyrrolidine-2-carboxamide (85 mg, 0.17 mmol, 1.10 eq, hydrochloride) was added to the mixture, and the mixture was stirred at 20° C. for 12 h. The mixture was diluted with water (50 mL) and extracted with ethyl acetate (25 mL×3). The combined organic layers were washed with brine (50 mL), dried over anhydrous sodium sulfate, filtered and the filtrate was concentrated under reduced pressure to give a residue. The residue was purified by preparative thin layer chromatography (dichloromethane: methanol=10:1) to give tert-butyl 3-(2-(((S)-1-(3-(3-(((S)-1-((2S,4R)-2-(((S)-1-(6-(2,6-difluorophenyl)pyridin-3-yl)ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate (130 mg, 0.10 mmol, 63% yield, 95% purity) as a white solid. LCMS (ESI, m/z): 1259.5 [M+H]⁺.

Step 12: (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)propanamido)-3,3-dimethylbutanoyl)-N—((S)-1-(6-(2,6-difluorophenyl)pyridin-3-yl)ethyl)-4-hydroxypyrrolidine-2-carboxamide (Example 31)

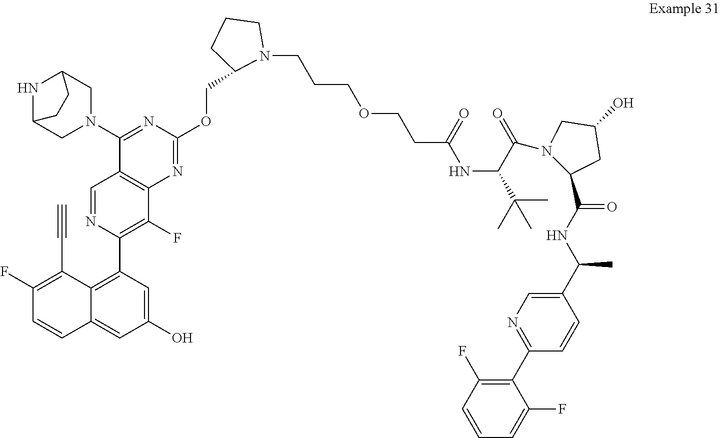 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(F)cccc6F)nc5)C(C)(C)C)nc4c3F)c12]

To a solution of tert-butyl 3-(2-(((S)-1-(3-(3-(((S)-1-((2S,4R)-2-(((S)-1-(6-(2,6-difluorophenyl)pyridin-3-yl)ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate (130 mg, 0.10 mmol, 1.00 eq) in dichloromethane (10 mL) was added trifluoroacetic acid (3.08 g, 0.03 mmol, 2 mL, 260.00 eq) at 20° C. and the mixture was stirred for 1 h. The mixture was concentrated under reduced pressure to give a residue. The residue was purified by preparative high liquid chromatography (column: Phenomenex Luna C18 150*25 mm*10 um; mobile phase: [water(TFA)-ACN]; B %: 18%-48%, 11 min) to give the desired product (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)propanamido)-3,3-dimethylbutanoyl)-N—((S)-1-(6-(2,6-difluorophenyl)pyridin-3-yl)ethyl)-4-hydroxypyrrolidine-2-carboxamide (58 mg, 0.05 mmol, 50% yield, 99% purity) as a yellow solid. LCMS (ESI, m/z): 1115.4 [M]⁺, ¹H NMR: (400 MHz, DMSO-d₆) δ 10.45-9.72 (m, 1H), 9.06 (s, 1H), 8.62 (d, J=2.0 Hz, 1H), 8.47 (d, J=7.2 Hz, 1H), 8.03-7.93 (m, 1H), 7.85-7.75 (m, 2H), 7.56-7.43 (m, 3H), 7.39 (d, J=2.0 Hz, 1H), 7.26-7.17 (m, 3H), 5.26-5.04 (m, 1H), 5.01-4.93 (m, 1H), 4.59-4.47 (m, 2H), 4.43-4.34 (m, 3H), 4.27 (d, J=1.6 Hz, 1H), 4.16-4.07 (m, 1H), 3.92 (d, J=2.4 Hz, 1H), 3.83 (s, 2H), 3.78-3.67 (m, 2H), 3.60-3.45 (m, 4H), 3.38 (t, J=6.0 Hz, 2H), 3.29 (s, 2H), 3.10-3.00 (m, 1H), 2.94-2.76 (m, 2H), 2.29-2.13 (m, 2H), 2.05-1.98 (m, 1H), 1.95-1.88 (m, 1H), 1.79 (s, 4H), 1.75-1.62 (m, 6H), 1.42 (d, J=6.8 Hz, 3H), 1.29-1.19 (m, 1H), 0.89 (d, J=2.8 Hz, 9H)

Example 32: (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)propanamido)-3,3-dimethylbutanoyl)-N—((R)-1-(2′,6′-difluoro-[1,1′-biphenyl]-4-yl)-2-hydroxyethyl)-4-hydroxypyrrolidine-2-carboxamide

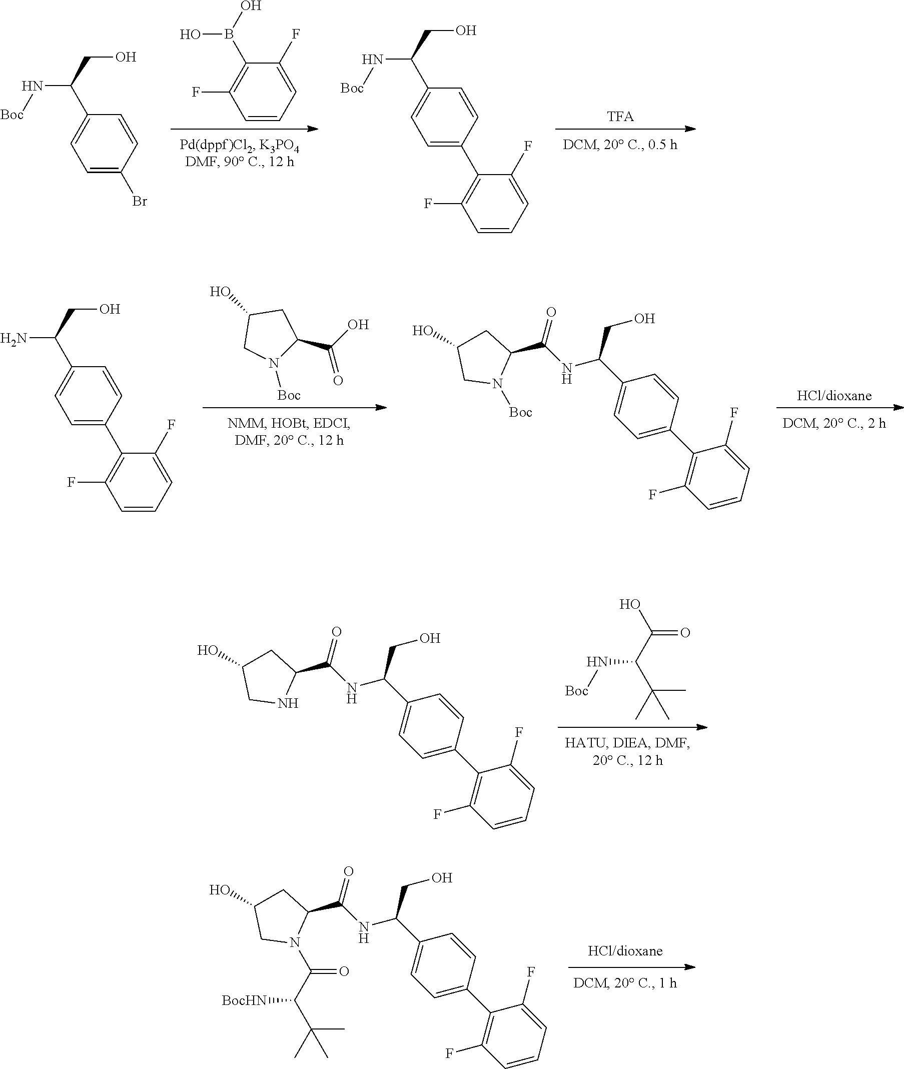

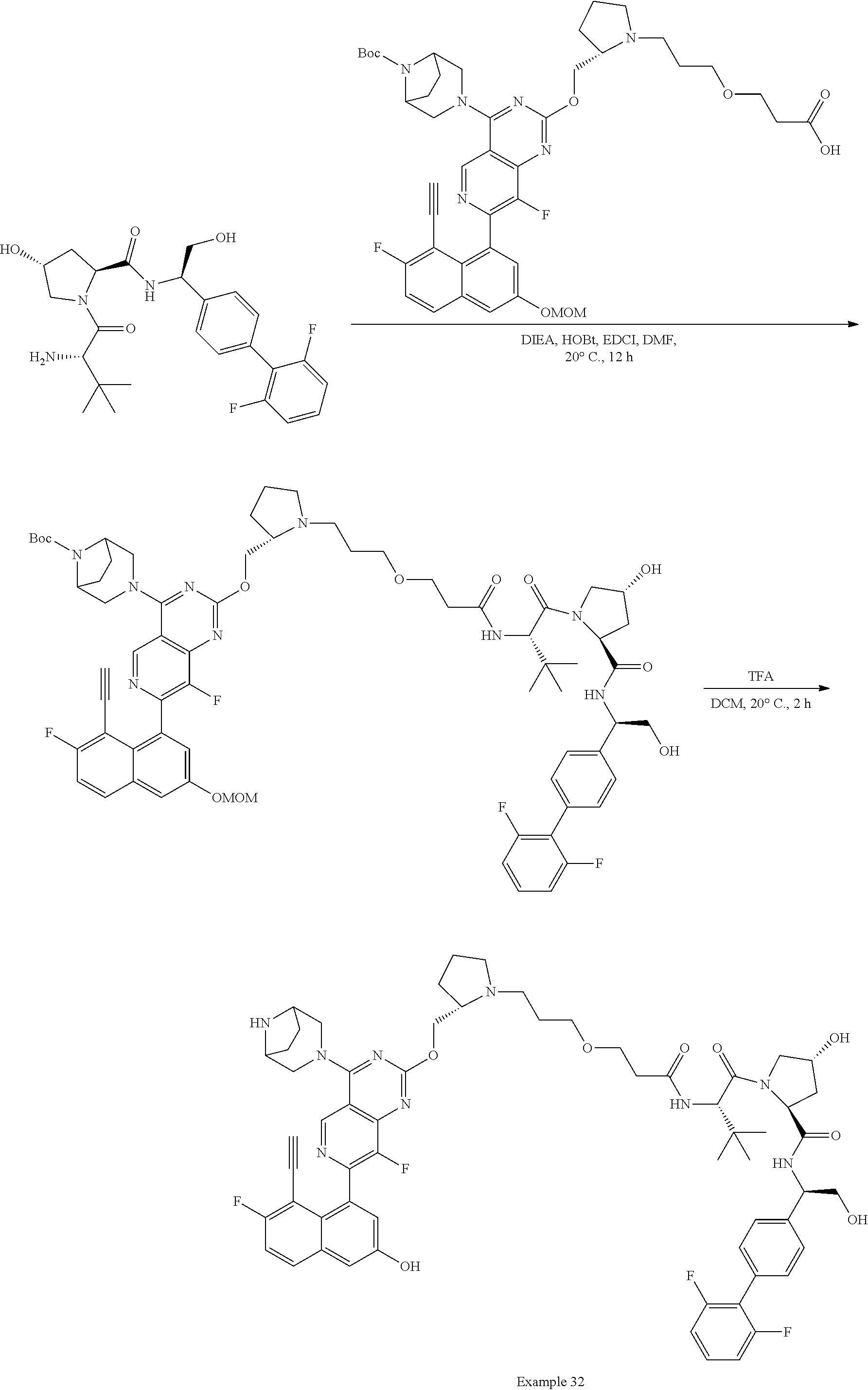

Step 1: tert-butyl (R)-(1-(2′,6′-difluoro-[1,1′-biphenyl]-4-yl)-2-hydroxyethyl)carbamate

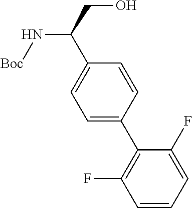 [SMILES: CC(C)(C)OC(=O)N[C@@H](CO)c1ccc(-c2c(F)cccc2F)cc1]

A mixture of tert-butyl N-[(1R)-1-(4-bromophenyl)-2-hydroxy-ethyl]carbamate (500 mg, 1.58 mmol, 1.00 eq), (2,6-difluorophenyl)boronic acid (500 mg, 3.16 mmol, 2.00 eq), [1,1′-bis(diphenylphosphino)ferrocene]dichloropalladium(II) (232 mg, 0.32 mmol, 0.20 eq), potassium phosphate (671 mg, 3.16 mmol, 2.00 eq) in water (2 mL) and tetrahydrofuran (10 mL) was degassed and purged with nitrogen for 3 times, and then the mixture was stirred at 70° C. for 12 h under nitrogen atmosphere. Water (50 mL) was added before the mixture was extracted by ethyl acetate (30 mL×3). The combined organic layers were evaporated under vacuum to get a residue. The residue was purified by preparative high liquid chromatography (column: Phenomenex luna C18 (250*70 mm, 10 um); mobile phase: [water(FA)-ACN]; B %: 40%-70%, 21 min) to give tert-butyl (R)-(1-(2′,6′-difluoro-[1,1′-biphenyl]-4-yl)-2-hydroxyethyl)carbamate (1.65 g, 4.72 mmol, 66% yield) as a white solid. LCMS (ESI, m/z): 372.1 [M+H]⁺.

Step 2: (R)-2-amino-2-(2′,6′-difluoro-[1,1′-biphenyl]-4-yl)ethan-1-ol

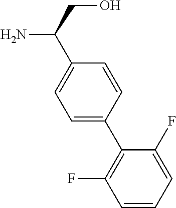 [SMILES: N[C@@H](CO)c1ccc(-c2c(F)cccc2F)cc1]

To a solution of tert-butyl (R)-(1-(2′,6′-difluoro-[1,1′-biphenyl]-4-yl)-2-hydroxyethyl)carbamate (900 mg, 2.58 mmol, 1.00 eq) in dichloromethane (15 mL) was added trifluoroacetic acid (4.62 g, 40.52 mmol, 15.73 eq). The mixture was stirred at 20° C. for 0.5 h. The reaction mixture was evaporated under reduced pressure to give the crude product (R)-2-amino-2-(2′,6′-difluoro-[1,1′-biphenyl]-4-yl)ethan-1-ol (900 mg, 2.48 mmol, 96% yield, trifluoroacetate) as a white solid. LCMS (ESI, m/z): 274.2 [M+Na]⁺.

Step 3: tert-butyl (2S,4R)-2-(((R)-1-(2′,6′-difluoro-[1,1′-biphenyl]-4-yl)-2-hydroxyethyl)carbamoyl)-4-hydroxypyrrolidine-1-carboxylate

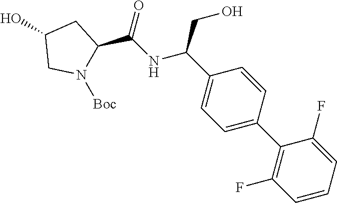 [SMILES: CC(C)(C)OC(=O)N1C[C@H](O)C[C@H]1C(=O)N[C@@H](CO)c1ccc(-c2c(F)cccc2F)cc1]

To a solution of (2S,4R)-1-tert-butoxycarbonyl-4-hydroxy-pyrrolidine-2-carboxylic acid (586 mg, 2.54 mmol, 1.05 eq) in N,N-dimethylformamide (10 mL) was added N-monomethyl morpholine (1.22 g, 12.07 mmol, 5.00 eq), N-(3-dimethylaminopropyl)-N-ethylcarbodiimide hydrochloride (695 mg, 3.62 mmol, 1.50 eq), 1-hydroxybenzotriazole (490 mg, 3.62 mmol, 1.50 eq) and (R)-2-amino-2-(2′,6′-difluoro-[1,1′-biphenyl]-4-yl)ethan-1-ol (690 mg, 2.41 mmol, 1.00 eq, hydrochloride). The mixture was stirred at 20° C. for 2 h. Water (50 mL) was added before the mixture was extracted by ethyl acetate (50 mL×3). The combined organic layers were evaporated under vacuum to get the residue. The residue was purified by preparative high liquid chromatography (column: Phenomenex luna C18 250*50 mm*10 um; mobile phase: [water(FA)-ACN]; B %: 30%-60%, 20 min) to give tert-butyl (2S,4R)-2-(((R)-1-(2′,6′-difluoro-[1,1′-biphenyl]-4-yl)-2-hydroxyethyl)carbamoyl)-4-hydroxypyrrolidine-1-carboxylate. LCMS (ESI, m/z): 463.2 [M+H]⁺.

Step 4: (2S,4R)—N—((R)-1-(2′,6′-difluoro-[1,1′-biphenyl]-4-yl)-2-hydroxyethyl)-4-hydroxypyrrolidine-2-carboxamide

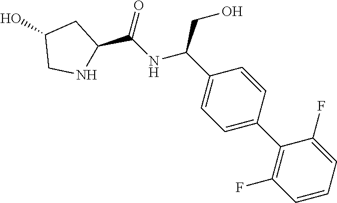 [SMILES: O=C(N[C@@H](CO)c1ccc(-c2c(F)cccc2F)cc1)[C@@H]1C[C@@H](O)CN1]

To a solution of tert-butyl (2S,4R)-2-(((R)-1-(2′,6′-difluoro-[1,1′-biphenyl]-4-yl)-2-hydroxyethyl)carbamoyl)-4-hydroxypyrrolidine-1-carboxylate (1.20 g, 2.59 mmol, 1.00 eq) in dichloromethane (15 mL) was added hydrochloric acid/dioxane (4 M, 6.0 mL, 9.25 eq). The mixture was stirred at 20° C. for 2 h. The reaction mixture was filtered and the filtrate was concentrated under reduced pressure to give (2S,4R)—N—((R)-1-(2′,6′-difluoro-[1,1′-biphenyl]-4-yl)-2-hydroxyethyl)-4-hydroxypyrrolidine-2-carboxamide (1.00 g, 2.51 mmol, 97% yield, hydrochloride) as a white solid, which was used into next step without further purification. LCMS (ESI, m/z): 363.3 [M+H]⁺

Step 5: tert-butyl ((S)-1-((2S,4R)-2-(((R)-1-(2′,6′-difluoro-[1,1′-biphenyl]-4-yl)-2-hydroxyethyl)carbamoyl)-4-hydroxypyrrolidin-1-yl)-3,3-dimethyl-1-oxobutan-2-yl)carbamate

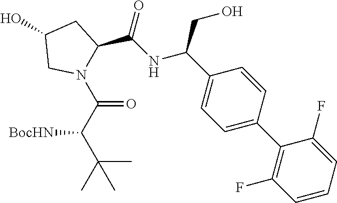 [SMILES: CC(C)(C)OC(=O)N[C@H](C(=O)N1C[C@H](O)C[C@H]1C(=O)N[C@@H](CO)c1ccc(-c2c(F)cccc2F)cc1)C(C)(C)C]

To a solution of (2S)-2-(tert-butoxycarbonylamino)-3,3-dimethyl-butanoic acid (580 mg, 2.51 mmol, 1.00 eq) in N,N-dimethylformamide (15 mL) was added 1-hydroxybenzotriazole (510 mg, 3.76 mmol, 1.50 eq), N-(3-dimethylaminopropyl)-N-ethylcarbodiimide hydrochloride (722 mg, 3.76 mmol, 1.50 eq), diisopropylethylamine (1.62 g, 12.55 mmol, 5.00 eq) and (2S,4R)—N—((R)-1-(2′,6′-difluoro-[1,1′-biphenyl]-4-yl)-2-hydroxyethyl)-4-hydroxypyrrolidine-2-carboxamide (1.00 g, 2.51 mmol, 1.00 eq, hydrochloride). The mixture was stirred at 20° C. for 2 h. Water (20 mL) was added before the mixture was extracted by ethyl acetate (10 mL×3). The combined organic layers were dried over sodium sulfate, evaporated under vacuum to get the residue. The residue was purified by preparative thin layer chromatography (dichloromethane/methanol =10/1) to give tert-butyl ((S)-1-((2S,4R)-2-(((R)-1-(2′,6′-difluoro-[1,1′-biphenyl]-4-yl)-2-hydroxyethyl)carbamoyl)-4-hydroxypyrrolidin-1-yl)-3,3-dimethyl-1-oxobutan-2-yl)carbamate (700 mg, 1.22 mmol, 48% yield) as a white solid. LCMS (ESI, m/z): 576.3 [M+H] ⁺

Step 6: (2S,4R)-1-((S)-2-amino-3,3-dimethylbutanoyl)-N—((R)-1-(2′,6′-difluoro-[1,1′-biphenyl]-4-yl)-2-hydroxyethyl)-4-hydroxypyrrolidine-2-carboxamide

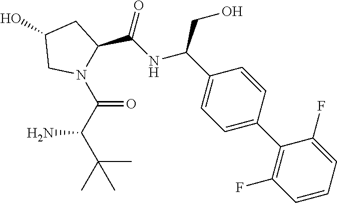 [SMILES: CC(C)(C)[C@H](N)C(=O)N1C[C@H](O)C[C@H]1C(=O)N[C@@H](CO)c1ccc(-c2c(F)cccc2F)cc1]

To a solution of tert-butyl ((S)-1-((2S,4R)-2-(((R)-1-(2′,6′-difluoro-[1,1′-biphenyl]-4-yl)-2-hydroxyethyl)carbamoyl)-4-hydroxypyrrolidin-1-yl)-3,3-dimethyl-1-oxobutan-2-yl)carbamate (700 mg, 1.22 mmol, 1.00 eq) in dichloromethane (10 mL) was added hydrochloric acid/dioxane (4 M, 5 mL, 1.00 eq). The mixture was stirred at 20° C. for 1 h. The mixture was evaporated under vacuum to give (2S,4R)-1-((S)-2-amino-3,3-dimethylbutanoyl)-N—((R)-1-(2′,6′-difluoro-[1,1′-biphenyl]-4-yl)-2-hydroxyethyl)-4-hydroxypyrrolidine-2-carboxamide (620 mg, 1.21 mmol, 100% yield, hydrochloride) as a white solid. LCMS (ESI, m/z): 476.3 [M+H]⁺

Step 7: tert-butyl 3-(2-(((S)-1-(3-(3-(((S)-1-((2S,4R)-2-(((R)-1-(2′,6′-difluoro-[1,1′-biphenyl]-4-yl)-2-hydroxyethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate

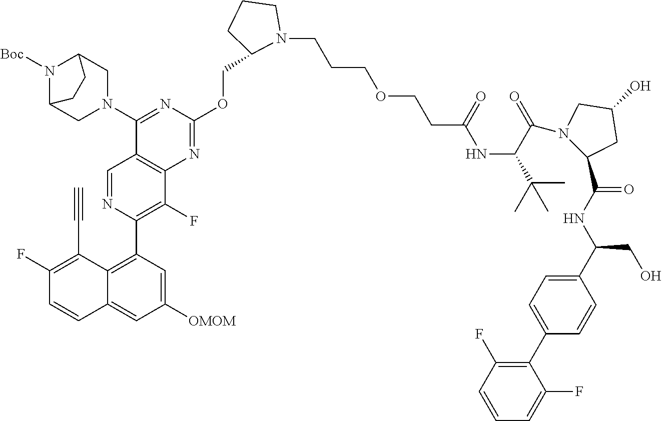

To a solution of 3-[3-[(2S)-2-[[4-(8-tert-butoxycarbonyl-3,8-diazabicyclo[3.2.1]octan-3-yl)-7-[8-ethynyl-7-fluoro-3-(methoxymethoxy)-1-naphthyl]-8-fluoro-pyrido[4,3-d]pyrimidin-2-yl]oxymethyl]pyrrolidin-1-yl]propoxy]propanoic acid (160 mg, 0.20 mmol, 1.00 eq) in N,N-dimethylformamide (4 mL) was added diisopropylethylamine (126 mg, 0.98 mmol, 5.00 eq) and 1-hydroxybenzotriazole (40 mg, 0.30 mmol, 1.50 eq), N-(3-dimethylaminopropyl)-N-ethylcarbodiimide hydrochloride (56 mg, 0.30 mmol, 1.50 eq) and (2S,4R)-1-((S)-2-amino-3,3-dimethylbutanoyl)-N—((R)-1-(2′,6′-difluoro-[1,1′-biphenyl]-4-yl)-2-hydroxyethyl)-4-hydroxypyrrolidine-2-carboxamide (100 mg, 0.20 mmol, 1.00 eq, hydrochloride). The mixture was stirred at 20° C. for 2 h. Water (50 mL) was added before the mixture was extracted by dichloromethane (20 mL×3). The combined organic layers were evaporated under vacuum to get the residue. The residue was purified by preparative thin layer chromatography on silica gel (dichloromethane/methanol=10/1) to give the product tert-butyl 3-(2-(((S)-1-(3-(3-(((S)-1-((2S,4R)-2-(((R)-1-(2′,6′-difluoro-[1,1′-biphenyl]-4-yl)-2-hydroxyethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate (180 mg, 0.14 mmol, 72% yield) as a white solid. LCMS (ESI, m/z): 1274.5 [M]⁺

Step 8: (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)propanamido)-3,3-dimethylbutanoyl)-N—((R)-1-(2′,6′-difluoro-[1,1′-biphenyl]-4-yl)-2-hydroxyethyl)-4-hydroxypyrrolidine-2-carboxamide (Example 32)

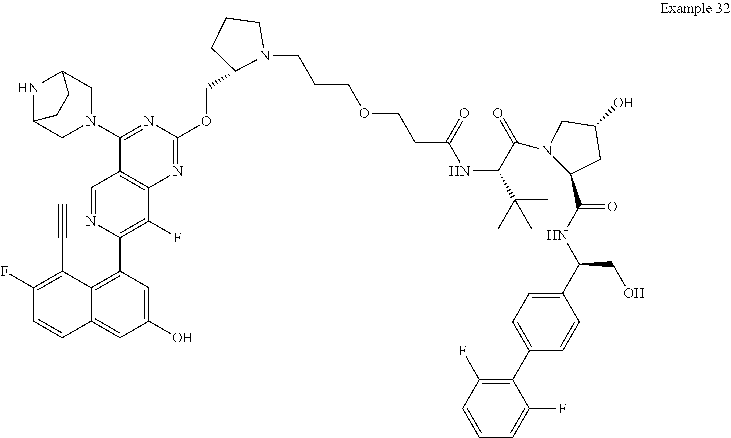 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](CO)c5ccc(-c6c(F)cccc6F)cc5)C(C)(C)C)nc4c3F)c12]

To a solution of tert-butyl 3-(2-(((S)-1-(3-(3-(((S)-1-((2S,4R)-2-(((R)-1-(2′,6′-difluoro-[1,1′-biphenyl]-4-yl)-2-hydroxyethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate (180 mg, 0.14 mmol, 1.00 eq) in dichloromethane (10 mL) was added trifluoroacetic acid (3.08 g, 27.01 mmol, 191.40 eq). The mixture was stirred at 20° C. for 2 h. Water (50 mL) was added before the mixture was extracted by dichloromethane (20 mL×3). The combined organic layers were evaporated under reduced pressure to get a residue. The residue was purified by preparative high liquid chromatography (column: Phenomenex luna C18 150*25 mm*10 um; mobile phase: [water(TFA)-ACN]; B %: 23%-53%, 11 min) to afford the desired product (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)propanamido)-3,3-dimethylbutanoyl)-N—((R)-1-(2′,6′-difluoro-[1,1′-biphenyl]-4-yl)-2-hydroxyethyl)-4-hydroxypyrrolidine-2-carboxamide (67.38 mg, 0.06 mmol, 42% yield, 100% purity). LCMS (ESI, m/z): 1130.2 [M]⁺, 1H NMR: (400 MHz, DMSO-d6) δ 10.19 (s, 1H), 9.06 (s, 1H), 8.43-8.35 (m, 1H), 7.98 (d, J=6.0, 9.2 Hz, 1H), 7.85 (d, J=7.2 Hz, 1H), 7.50-7.42 (m, 3H), 7.42-7.37 (m, 4H), 7.26-7.17 (m, 3H), 5.22-5.04 (m, 1H), 4.93-4.86 (m, 1H), 4.83-4.72 (m, 1H), 4.57-4.45 (m, 3H), 4.37 (d, J=11.2 Hz, 2H), 4.31-4.26 (m, 1H), 4.17-4.05 (m, 1H), 3.94 (s, 1H), 3.75 (s, 2H), 3.70 (d, J=12.4 Hz, 1H), 3.66-3.60 (m, 4H), 3.57-3.49 (m, 2H), 3.39 (d, J=3.2 Hz, 2H), 3.31 (s, 2H), 3.12-3.00 (m, 1H), 2.97-2.73 (m, 2H), 2.29-2.14 (m, 2H), 2.10-1.87 (m, 3H), 1.86-1.63 (m, 11H), 0.92 (d, J=2.0 Hz, 9H).

Example 33: Synthesis of (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)propanamido)-3,3-dimethylbutanoyl)-N—((S)-1-(4-((S)-2-cyanopyrrolidin-1-yl)phenyl)ethyl)-4-hydroxypyrrolidine-2-carboxamide

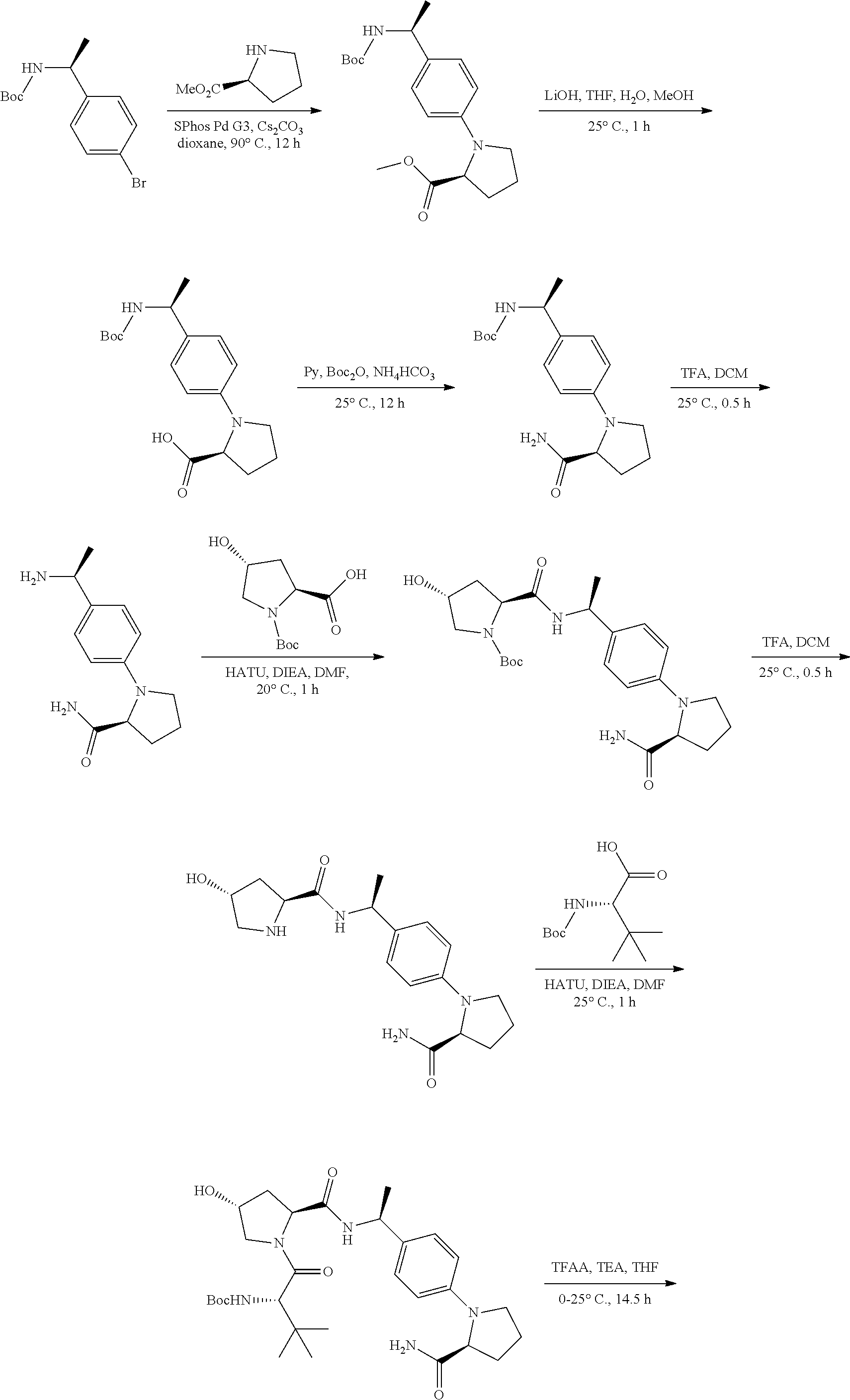

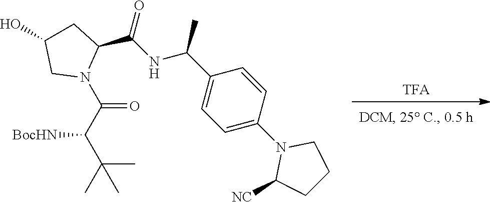 [SMILES: C[C@H](NC(=O)[C@@H]1C[C@@H](O)CN1C(=O)[C@@H](NC(=O)OC(C)(C)C)C(C)(C)C)c1ccc(N2CCC[C@H]2C#N)cc1 ; "TFA"]

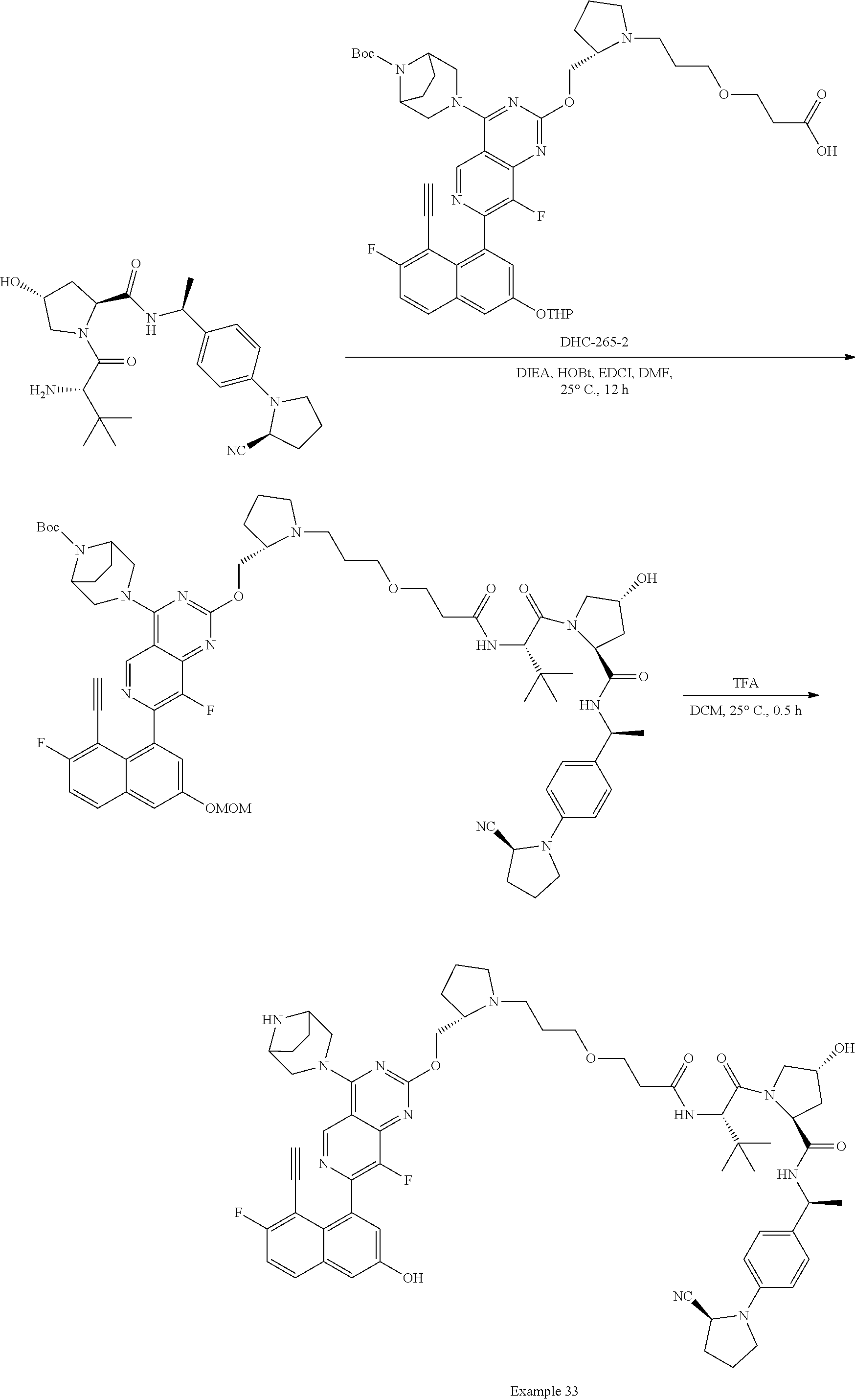

Step 1: methyl (4-((S)-1-((tert-butoxycarbonyl)amino)ethyl)phenyl)-L-prolinate

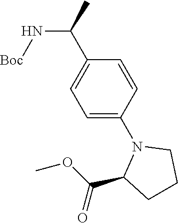 [SMILES: COC(=O)[C@@H]1CCCN1c1ccc([C@H](C)NC(=O)OC(C)(C)C)cc1]

To a solution of tert-butyl (S)-(1-(4-bromophenyl)ethyl)carbamate (5 g, 16.66 mmol), methyl L-prolinate (2.76 g, 16.66 mmol, hydrochloride) in dioxane (75 mL) was added dicyclohexyl-[2-(2,6-dimethoxyphenyl)phenyl]phosphane; methanesulfonate; (2-phenylanilino)palladium(1+) (1.30 g, 1.67 mmol) and cesium carbonate (21.71 g, 66.62 mmol). The mixture was stirred at 90° C. for 12 h. The reaction mixture was quenched by adding saturated aqueous ammonium chloride (100 mL), diluted with ethyl acetate (100 mL) and extracted with ethyl acetate (100 mL×3). The combined organic layers were washed with brine (300 mL), dried over sodium sulfate, filtered and concentrated under reduced pressure to give a residue. The residue was purified by silica gel column chromatography to afford the desired product methyl (4-((S)-1-((tert-butoxycarbonyl)amino)ethyl)phenyl)-L-prolinate (2.32 g, 6.66 mmol, 40% yield) as a yellow oil. LCMS (ESI, m/z): 349.0 [M+H]⁺, ¹H NMR (400 MHz, CHLOROFORM-d) δ 7.09 (d, J=8.4 Hz, 2H), 6.43 (d, J=8.8 Hz, 2H), 4.66-4.58 (m, 1H), 4.18-4.14 (m, 1H), 3.64 (s, 3H), 3.53-3.47 (m, 1H), 3.27 (d, J=8.0 Hz, 1H), 2.24-2.14 (m, 1H), 2.13-2.05 (m, 2H), 2.01-1.96 (m, 2H), 1.35 (s, 12H)

Step 2: (4-((S)-1-((tert-butoxycarbonyl)amino)ethyl)phenyl)-L-proline

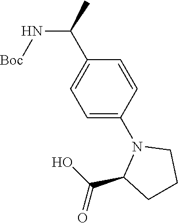 [SMILES: C[C@H](NC(=O)OC(C)(C)C)c1ccc(N2CCC[C@H]2C(=O)O)cc1]

To a solution of methyl (4-((S)-1-((tert-butoxycarbonyl)amino)ethyl)phenyl)-L-prolinate (3 g, 8.61 mmol) in water (5 mL), tetrahydrofuran (10 mL) and methanol (10 mL) was added lithium hydroxide (1.08 g, 25.83 mmol). The mixture was stirred at 25° C. for 1 h. The reaction mixture was quenched by adding aqueous hydrochloride (1 M, 15 mL), and then extracted with ethyl acetate (20 mL×3). The combined organic layers were washed with brine (50 mL), dried over sodium sulfate, filtered and concentrated under reduced pressure to afford the crude product (4-((S)-1-((tert-butoxycarbonyl)amino)ethyl)phenyl)-L-proline (2.8 g, 8.37 mmol, 97% yield) as a red oil. LCMS (ESI, m/z): 335.0 [M+H]⁺.

Step 3: tert-butyl ((S)-1-(4-((S)-2-carbamoylpyrrolidin-1-yl)phenyl)ethyl)carbamate

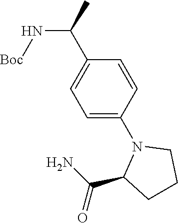 [SMILES: C[C@H](NC(=O)OC(C)(C)C)c1ccc(N2CCC[C@H]2C(N)=O)cc1]

To a solution of ((4-((S)-1-((tert-butoxycarbonyl)amino)ethyl)phenyl)-L-proline (2.8 g, 8.37 mmol) in dioxane (30 mL) was added pyridine (1.32 g, 16.75 mmol, 1.4 mL), ammonium hydrogen carbonate (1.99 g, 25.12 mmol, 2.1 mL) and di-tert-butyl dicarbonate (2.92 g, 13.40 mmol, 3.1 mL). The mixture was stirred at 25° C. for 16 h. The reaction mixture was quenched by adding water (20 mL), and then extracted with ethyl acetate (30 mL×3). The combined organic layers were washed with brine (50 mL), dried over sodium sulfate, filtered and concentrated under reduced pressure to give a residue. The residue was purified by prep-HPLC (column: Phenomenex luna C18 250*80 mm*10 um; mobile phase: [water(FA)-ACN]; B %: 20%-50%, 20 min) to afford the desired product tert-butyl ((S)-1-(4-((S)-2-carbamoylpyrrolidin-1-yl)phenyl)ethyl)carbamate (1.8 g, 5.40 mmol, 64% yield) as a yellow solid. LCMS (ESI, m/z): 334.4 [M+H]⁺, ¹H NMR (400 MHz, DMSO-d₆) δ 7.31-7.26 (m, 1H), 7.20-7.14 (m, 1H), 7.09 (d, J=8.4 Hz, 2H), 7.00 (s, 1H), 6.41 (d, J=8.4 Hz, 2H), 4.54-4.45 (m, 1H), 3.81 (s, 1H), 3.57-3.50 (m, 1H), 3.15 (q, J=8.0 Hz, 1H), 2.17 (br d, J=1.2 Hz, 1H), 2.00-1.91 (m, 3H), 1.36 (s, 9H), 1.25 (d, J=7.2 Hz, 3H).

Step 4: (S)-1-(4-((S)-1-aminoethyl)phenyl)pyrrolidine-2-carboxamide

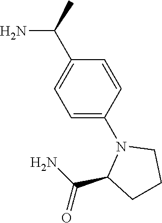 [SMILES: C[C@H](N)c1ccc(N2CCC[C@H]2C(N)=O)cc1]

To a solution of tert-butyl ((S)-1-(4-((S)-2-carbamoylpyrrolidin-1-yl)phenyl)ethyl)carbamate (500 mg, 1.50 mmol) in dichloromethane (2 mL) was added trifluoroacetic acid (6.16 g, 54.02 mmol, 4 mL). The mixture was stirred at 25° C. for 0.5 h. The reaction mixture was concentrated under reduced pressure to remove the solvent to afford the crude product (S)-1-(4-((S)-1-aminoethyl)phenyl)pyrrolidine-2-carboxamide (500 mg, 1.44 mmol, 96% yield, trifluoroacetate) as a yellow oil.

Step 5: tert-butyl (2S,4R)-2-(((S)-1-(4-((S)-2-carbamoylpyrrolidin-1-yl)phenyl)ethyl)carbamoyl)-4-hydroxypyrrolidine-1-carboxylate

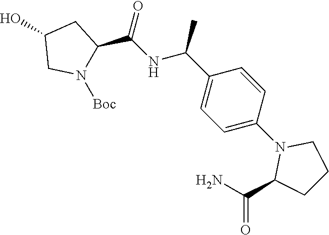 [SMILES: C[C@H](NC(=O)[C@@H]1C[C@@H](O)CN1C(=O)OC(C)(C)C)c1ccc(N2CCC[C@H]2C(N)=O)cc1]

To a solution of (S)-1-(4-((S)-1-aminoethyl)phenyl)pyrrolidine-2-carboxamide (500 mg, 1.44 mmol, trifluoroacetate), (S)-2-((tert-butoxycarbonyl)amino)-3,3-dimethylbutanoic acid (499 mg, 2.16 mmol) in dimethylformamide (5 mL) was added N,N-diisopropylethylamine (744 mg, 5.76 mmol, 1.00 mL) and [dimethylamino(triazolo [4,5-b]pyridin-3-yloxy)methylene]-dimethyl-ammonium; hexafluorophosphate (821 mg, 2.16 mmol). The mixture was stirred at 20° C. for 0.5 h. The reaction mixture was quenched by adding water (20 mL), and then diluted with ethyl acetate (20 mL) and extracted with ethyl acetate (20 mL×3). The combined organic layers were washed with brine (50 mL×2), dried over sodium sulfate, filtered and concentrated under reduced pressure to give a residue. The residue was purified by prep-HPLC (column: Phenomenex luna C18 150*40 mm*15 um; mobile phase: [water(TFA)-ACN]; B %: 18%-48%, 10 min) to afford the desired product tert-butyl (2S,4R)-2-(((S)-1-(4-((S)-2-carbamoylpyrrolidin-1-yl)phenyl)ethyl)carbamoyl)-4-hydroxypyrrolidine-1-carboxylate (500 mg, 1.12 mmol, 78% yield) as an off-white solid. LCMS (ESI, m/z): 445.2 [M+H]⁺.

Step 6: (2S,4R)—N—((S)-1-(4-((S)-2-carbamoylpyrrolidin-1-yl)phenyl)ethyl)-4-hydroxypyrrolidine-2-carboxamide

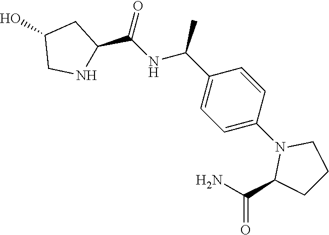 [SMILES: C[C@H](NC(=O)[C@@H]1C[C@@H](O)CN1)c1ccc(N2CCC[C@H]2C(N)=O)cc1]

To a solution of tert-butyl (2S,4R)-2-(((S)-1-(4-((S)-2-carbamoylpyrrolidin-1-yl)phenyl)ethyl)carbamoyl)-4-hydroxypyrrolidine-1-carboxylate (500 mg, 1.12 mmol) in dichloromethane (5 mL) was added trifluoroacetic acid (8.80 g, 77.18 mmol, 5.71 mL). The mixture was stirred at 25° C. for 0.5 h. The reaction mixture was concentrated under reduced pressure to remove solvent to afford the crude product (2S,4R)—N—((S)-1-(4-((S)-2-carbamoylpyrrolidin-1-yl)phenyl)ethyl)-4-hydroxypyrrolidine-2-carboxamide (500 mg, 1.09 mmol, 97% yield, trifluoroacetate) as a yellow oil.

Step 7: tert-butyl ((S)-1-((2S,4R)-2-(((S)-1-(4-((S)-2-carbamoylpyrrolidin-1-yl)phenyl)ethyl)carbamoyl)-4-hydroxypyrrolidin-1-yl)-3,3-dimethyl-1-oxobutan-2-yl)carbamate

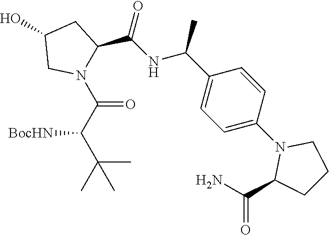 [SMILES: C[C@H](NC(=O)[C@@H]1C[C@@H](O)CN1C(=O)[C@@H](NC(=O)OC(C)(C)C)C(C)(C)C)c1ccc(N2CCC[C@H]2C(N)=O)cc1]

To a solution of (2S,4R)—N—((S)-1-(4-((S)-2-carbamoylpyrrolidin-1-yl)phenyl)ethyl)-4-hydroxypyrrolidine-2-carboxamide (500 mg, 1.09 mmol, trifluoroacetate), (S)-2-((tert-butoxycarbonyl)amino)-3,3-dimethylbutanoic acid (376.73 mg, 1.63 mmol) in dimethylformamide (7 mL) was added [dimethylamino(triazolo[4,5-b]pyridin-3-yloxy)methylene]-dimethyl-ammonium; hexafluorophosphate (619.34 mg, 1.63 mmol) and N,N-diisopropylethylamine (561.38 mg, 4.34 mmol, 0.8 mL). The mixture was stirred at 25° C. for 0.5 h. The mixture was purified by prep-HPLC (column: Phenomenex Synergi Max-RP 250*50 mm*10 um; mobile phase: [water(FA)-ACN]; B %: 30%-55%, 20 min) to afford the desired product tert-butyl ((S)-1-((2S,4R)-2-(((S)-1-(4-((S)-2-carbamoylpyrrolidin-1-yl)phenyl)ethyl)carbamoyl)-4-hydroxypyrrolidin-1-yl)-3,3-dimethyl-1-oxobutan-2-yl)carbamate (450 mg, 0.80 mmol, 74% yield) as an off-white solid. LCMS (ESI, m/z): 559.7 [M−H]⁺. ¹H NMR (400 MHz, CHLOROFORM-d) δ 7.41 (br d, J=7.6 Hz, 1H), 7.23 (d, J=8.8 Hz, 2H), 6.64 (d, J=8.8 Hz, 2H), 6.45 (br d, J=3.2 Hz, 1H), 5.37 (br s, 1H), 5.24 (br d, J=8.8 Hz, 1H), 4.98 (t, J=7.2 Hz, 1H), 4.75 (t, J=8.0 Hz, 1H), 4.51 (br s, 1H), 4.14 (q, J=7.2 Hz, 2H), 4.00 (dd, J=4.8, 7.2 Hz, 1H), 3.69-3.63 (m, 1H), 3.60-3.54 (m, 1H), 3.24 (br d, J=7.2 Hz, 1H), 2.65-2.57 (m, 1H), 2.33-2.26 (m, 2H), 2.06-2.00 (m, 2H), 1.65 (br d, J=4.4 Hz, 2H), 1.46-1.43 (m, 9H), 1.42-1.41 (m, 3H), 1.06 (s, 9H).

Step 8: tert-butyl ((S)-1-((2S,4R)-2-(((S)-1-(4-((S)-2-cyanopyrrolidin-1-yl)phenyl)ethyl)carbamoyl)-4-hydroxypyrrolidin-1-yl)-3,3-dimethyl-1-oxobutan-2-yl)carbamate

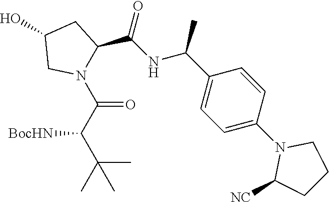 [SMILES: C[C@H](NC(=O)[C@@H]1C[C@@H](O)CN1C(=O)[C@@H](NC(=O)OC(C)(C)C)C(C)(C)C)c1ccc(N2CCC[C@H]2C#N)cc1]

A mixture of tert-butyl ((S)-1-((2S,4R)-2-(((S)-1-(4-((S)-2-carbamoylpyrrolidin-1-yl)phenyl)ethyl)carbamoyl)-4-hydroxypyrrolidin-1-yl)-3,3-dimethyl-1-oxobutan-2-yl)carbamate (160 mg, 0.28 mmol), triethylamine (64 mg, 0.63 mmol, 0.1 mL) in tetrahydrofuran (10 mL) was degassed and purged with nitrogen for 3 times, and then the mixture was stirred at 0° C. for 0.5 h under nitrogen atmosphere. Then (2,2,2-trifluoroacetyl) 2,2,2-trifluoroacetate (90 mg, 0.43 mmol, 0.1 mL) was added and the mixture was stirred at 0° C. for 2 h. Then the mixture was stirred at 25° C. for 16 h. The reaction mixture was quenched by adding saturated aqueous sodium bicarbonate (20 mL), and then extracted with ethyl acetate (20 mL×3). The combined organic layers were washed with brine (50 mL), dried over sodium sulfate, filtered and concentrated under reduced pressure to give a residue. The residue was purified by prep-TLC (eluent: DCM/MeOH=10/1) to afford the desired product tert-butyl ((S)-1-((2S,4R)-2-(((S)-1-(4-((S)-2-cyanopyrrolidin-1-yl)phenyl)ethyl)carbamoyl)-4-hydroxypyrrolidin-1-yl)-3,3-dimethyl-1-oxobutan-2-yl)carbamate (100 mg, 0.18 mmol, 61% yield) as a yellow solid. LCMS (ESI, m/z): 542.4 [M+H]⁺.

Step 9: (2S,4R)-1-((S)-2-amino-3,3-dimethylbutanoyl)-N—((S)-1-(4-((S)-2-cyanopyrrolidin-1-yl)phenyl)ethyl)-4-hydroxypyrrolidine-2-carboxamide

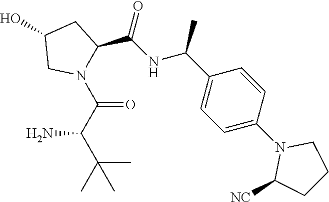 [SMILES: C[C@H](NC(=O)[C@@H]1C[C@@H](O)CN1C(=O)[C@@H](N)C(C)(C)C)c1ccc(N2CCC[C@H]2C#N)cc1]

To a solution of tert-butyl ((S)-1-((2S,4R)-2-(((S)-1-(4-((S)-2-cyanopyrrolidin-1-yl)phenyl)ethyl)carbamoyl)-4-hydroxypyrrolidin-1-yl)-3,3-dimethyl-1-oxobutan-2-yl)carbamate (100 mg, 0.18 mmol) in dichloromethane (1 mL) was added trifluoroacetic acid (1.54 g, 13.51 mmol, 1 mL). The mixture was stirred at 25° C. for 0.5 h. The reaction mixture was concentrated under reduced pressure to remove solvent to afford the crude product (2S,4R)-1-((S)-2-amino-3,3-dimethylbutanoyl)-N—((S)-1-(4-((S)-2-cyanopyrrolidin-1-yl)phenyl)ethyl)-4-hydroxypyrrolidine-2-carboxamide (100 mg, 0.18 mmol, 97% yield, trifluoroacetate) as a yellow oil. LCMS (ESI, m/z): 442.2 [M+H]⁺.

Step 10: tert-butyl 3-(2-(((S)-1-(3-(3-(((S)-1-((2S,4R)-2-(((S)-1-(4-((S)-2-cyanopyrrolidin-1-yl)phenyl)ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate

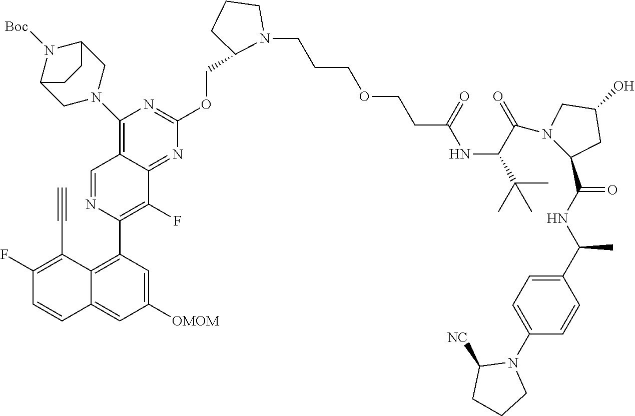

To a solution of ((2S,4R)-1-((S)-2-amino-3,3-dimethylbutanoyl)-N—((S)-1-(4-((S)-2-cyanopyrrolidin-1-yl)phenyl)ethyl)-4-hydroxypyrrolidine-2-carboxamide (78 mg, 0.14 mmol, trifluoroacetate), 3-(3-((2S)-2-(((4-(8-(tert-butoxycarbonyl)-3,8-diazabicyclo[3.2.1]octan-3-yl)-7-(8-ethynyl-7-fluoro-3-((tetrahydro-2H-pyran-2-yl)oxy)naphthalen-1-yl)-8-fluoropyrido[4,3-d]pyrimidin-2-yl)oxy)methyl)pyrrolidin-1-yl)propoxy)propanoic acid (120 mg, 0.14 mmol) in dimethylformamide (1 mL) was added N,N-diisopropylethylamine (72 mg, 0.56 mmol, 0.1 mL), 1-hydroxybenzotriazole (56 mg, 0.42 mmol) and 1-(3-dimethylaminopropyl)-3-ethylcarbodiimide hydrochloride (80 mg, 0.42 mmol). The mixture was stirred at 25° C. for 16 h. The reaction mixture was quenched by addition water (5 mL), and then extracted with ethyl acetate (10 mL×3). The combined organic layers were washed with brine (20 mL), dried over sodium sulfate, filtered and concentrated under reduced pressure to give a residue. The residue was purified by prep-HPLC (column: Phenomenex Synergi C18 150*25 mm*10 um; mobile phase: [water(FA)-ACN]; B %: 37%-55%, 9 min) to afford the desired product tert-butyl 3-(2-(((S)-1-(3-(3-(((S)-1-((2S,4R)-2-(((S)-1-(4-((S)-2-cyanopyrrolidin-1-yl)phenyl)ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate (25 mg, 0.02 mmol, 14% yield) as a white solid. LCMS (ESI, m/z): 1239.5 [M−H]⁺.

Step 11: (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)propanamido)-3,3-dimethylbutanoyl)-N—((S)-1-(4-((S)-2-cyanopyrrolidin-1-yl)phenyl)ethyl)-4-hydroxypyrrolidine-2-carboxamide (Example 33)

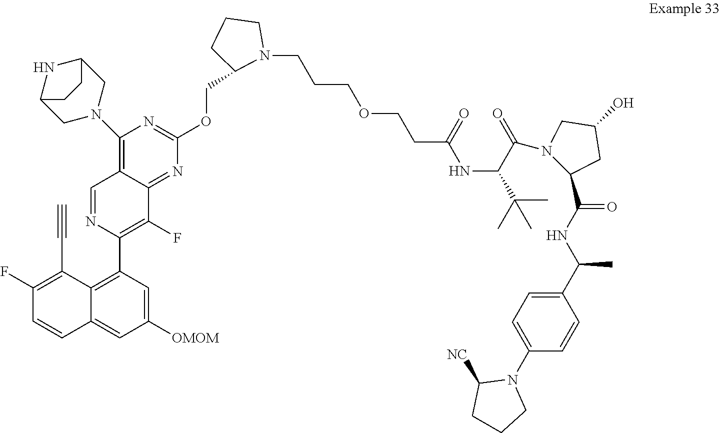

To a solution of tert-butyl 3-(2-(((S)-1-(3-(3-(((S)-1-((2S,4R)-2-(((S)-1-(4-((S)-2-cyanopyrrolidin-1-yl)phenyl)ethyl)carbamoyl)-4-hydroxypyrrolidin-1-yl)-3,3-dimethyl-1-oxobutan-2-yl)amino)-3-oxopropoxy)propyl)pyrrolidin-2-yl)methoxy)-7-(8-ethynyl-7-fluoro-3-(methoxymethoxy)naphthalen-1-yl)-8-fluoropyrido[4,3-d]pyrimidin-4-yl)-3,8-diazabicyclo[3.2.1]octane-8-carboxylate (15 mg, 0.01 mmol) in dichloromethane (0.5 mL) was added trifluoroacetic acid (770 mg, 6.75 mmol, 0.5 mL). The mixture was stirred at 25° C. for 0.5 h. The reaction mixture was concentrated under reduced pressure to remove the solvent. The residue was purified by prep-HPLC (column: Phenomenex Synergi C18 150*25 mm*10 um; mobile phase: [water(FA)-ACN]; B %: 13%-33%, 10 min) to afford the desired product (2S,4R)-1-((2S)-2-(3-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)propanamido)-3,3-dimethylbutanoyl)-N—((S)-1-(4-((S)-2-cyanopyrrolidin-1-yl)phenyl)ethyl)-4-hydroxypyrrolidine-2-carboxamide (8.4 mg, 0.01 mmol, 65% yield) as a yellow solid. LCMS (ESI, m/z): 1095.6 [M−H]⁺. ¹H NMR (400 MHz, DMSO-d₆) δ 9.05 (s, 1H), 8.20 (s, 2H), 7.98 (dd, J=6.0, 9.2 Hz, 1H), 7.85-7.79 (m, 1H), 7.47 (t, J=9.2 Hz, 1H), 7.40 (d, J=2.4 Hz, 1H), 7.20-7.14 (m, 3H), 6.66 (d, J=8.4 Hz, 2H), 4.85-4.76 (m, 2H), 4.55-4.48 (m, 2H), 4.42-4.24 (m, 5H), 4.09 (br d, J=10.4 Hz, 1H), 3.93 (d, J=3.2 Hz, 1H), 3.69 (br s, 2H), 3.62 (br s, 5H), 3.58-3.55 (m, 3H), 3.34-3.28 (m, 8H), 3.08-3.02 (m, 2H), 2.92-2.76 (m, 3H), 2.15-2.06 (m, 2H), 1.97-1.89 (m, 2H), 1.75-1.66 (m, 8H), 1.35-1.28 (m, 3H), 1.09-1.04 (m, 1H), 0.95-0.89 (m, 9H).

Example 34: (2S,4R)-1-((2S)-2-(4-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)butanamido)-3,3-dimethylbutanoyl)-N—((S)-1-(2′,6′-difluoro-[1,1′-biphenyl]-4-yl)ethyl)-4-hydroxypyrrolidine-2-carboxamide

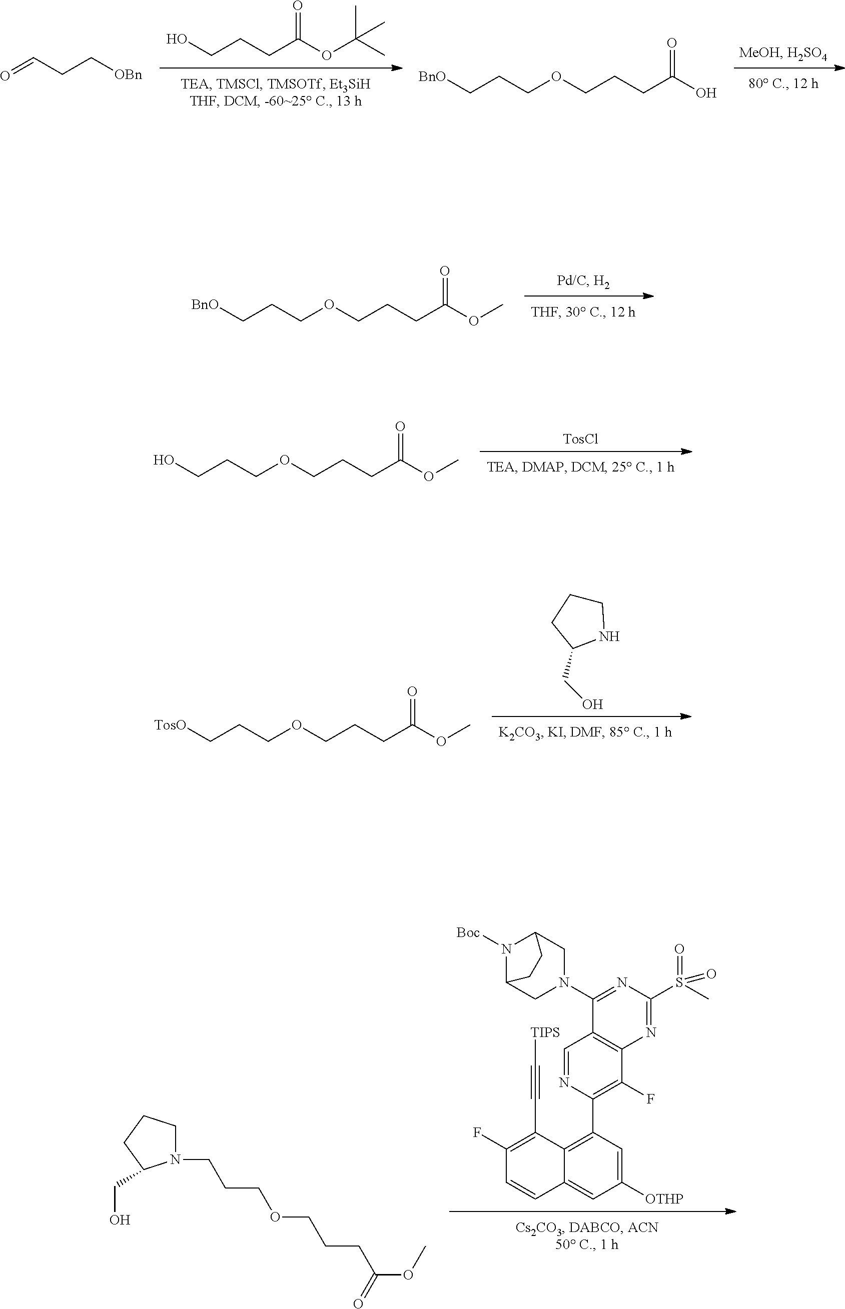

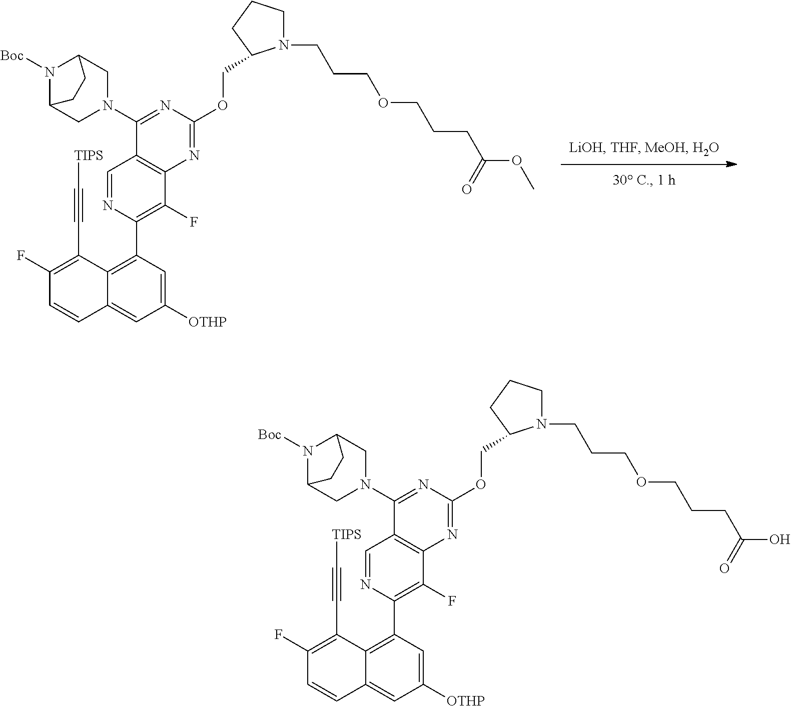

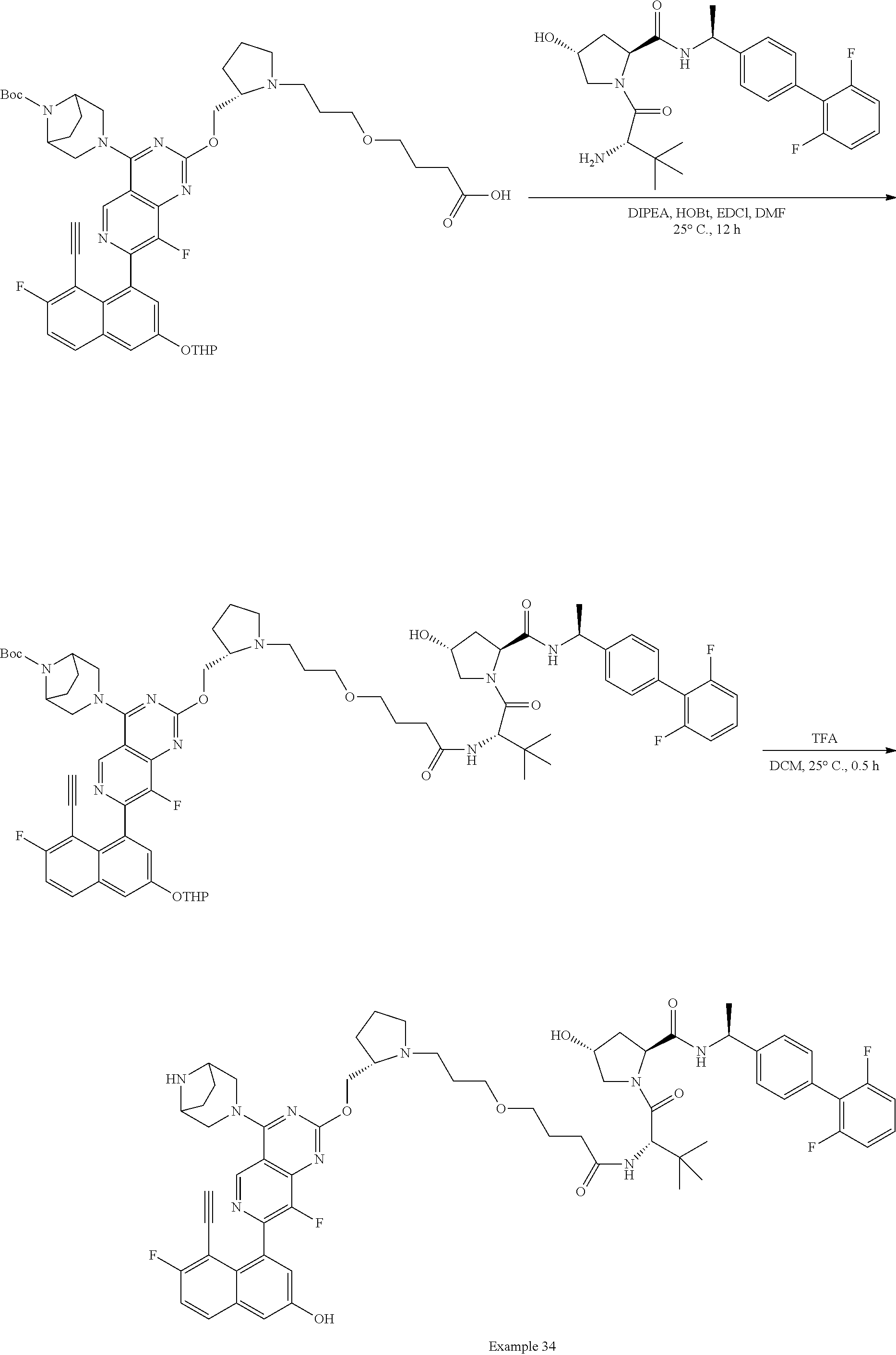

Step 1: 4-(3-(benzyloxy)propoxy)butanoic acid

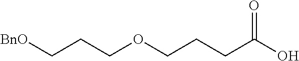 [SMILES: O=C(O)CCCOCCCOCc1ccccc1]

To a solution of tert-butyl 4-hydroxybutanoate (4 g, 24.97 mmol) in tetrahydrofuran (80 mL) was added trimethylchlorosilane (2.98 g, 27.46 mmol, 3.5 mL) and triethylamine (2.91 g, 28.71 mmol, 4.0 mL) at 0° C. The mixture was warmed to 25° C. and stirred at 25° C. for 1 h. The reaction mixture was filtered and the filtrate was concentrated under reduced pressure to give a residue. Then to a stirred solution of above residue and 3-benzyloxypropanal (4.10 g, 24.97 mmol) in dichloromethane (40 mL) was added triethylsilane (3.34 g, 28.71 mmol, 4.6 mL), trimethylsilyl trifluoromethanesulfonate (2.77 g, 12.48 mmol, 2.3 mL) dropwise at −60° C., the mixture was stirred at 25° C. in nitrogen atmosphere overnight. The reaction mixture was quenched by adding saturated aqueous sodium bicarbonate (100 mL), and then diluted with dichloromethane (100 mL) and extracted with dichloromethane (100 mL×3). The combined organic layers were washed with water (200 mL), dried over sodium sulfate, filtered and concentrated under reduced pressure to give a residue. The residue was purified by silica gel column chromatography (eluent: PE/EtOAc=4/1) to afford the desired product 4-(3-(benzyloxy)propoxy)butanoic acid (1.6 g, 6.34 mmol, 25% yield) as a colorless oil.

Step 2: methyl 4-(3-(benzyloxy)propoxy)butanoate

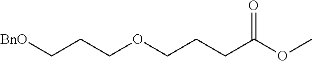 [SMILES: COC(=O)CCCOCCCOCc1ccccc1]

To a solution of 4-(3-(benzyloxy)propoxy)butanoic acid (1.6 g, 6.34 mmol) in methanol (30 mL) was added sulfuric acid (18 M, 3.5 mL) at 0° C. The mixture was stirred at 80° C. for 16 h. The reaction mixture was quenched by adding saturated aqueous sodium bicarbonate (10 mL), and then extracted with ethyl acetate (10 mL×3). The combined organic layers were washed with brine (20 mL), dried over sodium sulfate, filtered and concentrated under reduced pressure to afford the crude product methyl 4-(3-(benzyloxy)propoxy)butanoate (1.5 g, 5.63 mmol, 89% yield) as a white solid. LCMS (ESI, m/z): 267.2 [M+H]⁺.

Step 3: methyl 4-(3-hydroxypropoxy)butanoate

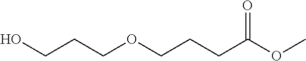

To a solution of methyl 4-(3-(benzyloxy)propoxy)butanoate (1.5 g, 5.63 mmol) in tetrahydrofuran (15 mL) was added palladium on activated carbon (300 mg, 5% purity, 50% in water) under nitrogen atmosphere. The suspension was degassed and purged with hydrogen for 3 times. The mixture was stirred under hydrogen (50 Psi) at 25° C. for 16 h. The reaction mixture was filtered and the filtrate was concentrated under reduced pressure to afford the crude product methyl 4-(3-hydroxypropoxy)butanoate (0.9 g, 5.11 mmol, 90% yield) as a colorless oil.

Step 4: methyl 4-(3-(tosyloxy)propoxy)butanoate

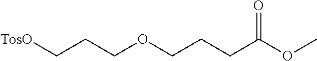

To a solution of methyl 4-(3-hydroxypropoxy)butanoate (0.9 g, 5.11 mmol) in dichloromethane (10 mL) was added p-toluenesulfonyl chloride (1.46 g, 7.66 mmol) and triethylamine (1.55 g, 15.32 mmol, 2.1 mL) and 4-dimethylaminopyridine (62 mg, 0.51 mmol). The mixture was stirred at 25° C. for 1 h. The reaction mixture was quenched by adding water (50 mL), and then diluted with dichloromethane (50 mL), extracted with dichloromethane (40 mL×3). The combined organic layers were washed with brine (40 mL×2), dried over sodium sulfate, filtered and concentrated under reduced pressure to give a residue. The residue was purified by silica gel column chromatography to afford the desired product methyl 4-(3-(tosyloxy)propoxy)butanoate (380 mg, 1.15 mmol, 22% yield) as a colorless oil. ¹H NMR (400 MHz, CHLOROFORM-d) δ 7.81 (d, J=8.0 Hz, 2H), 7.36 (d, J=8.0 Hz, 2H), 4.13 (t, J=6.4 Hz, 2H), 3.68 (s, 3H), 3.43 (t, J=6.0 Hz, 2H), 3.36 (t, J=6.0 Hz, 2H), 2.46 (s, 3H), 2.34 (t, J=7.2 Hz, 2H), 1.89 (t, J=6.0 Hz, 2H), 1.86-1.78 (m, 2H)

Step 5: methyl (S)-4-(3-(2-(hydroxymethyl)pyrrolidin-1-yl)propoxy)butanoate

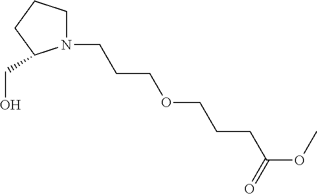 [SMILES: COC(=O)CCCOCCCN1CCC[C@H]1CO]

To a solution of methyl 4-(3-(tosyloxy)propoxy)butanoate (300 mg, 0.91 mmol) and [(2S)-pyrrolidin-2-yl]methanol (183 mg, 1.82 mmol) in acetonitrile (3 mL) was added potassium iodide (15 mg, 0.09 mmol) and potassium carbonate (376 mg, 2.72 mmol). The mixture was stirred at 80° C. for 1 h. The reaction mixture was quenched by adding water (5 mL), and then extracted with ethyl acetate (5 mL×3). The combined organic layers were washed with brine (5 mL) dried over sodium sulfate, filtered and concentrated under reduced pressure to afford the crude product methyl (S)-4-(3-(2-(hydroxymethyl)pyrrolidin-1-yl)propoxy)butanoate (120 mg, 0.46 mmol, 50% yield) as a yellow oil.

Step 6: tert-butyl 3-(8-fluoro-7-(7-fluoro-3-((tetrahydro-2H-pyran-2-yl)oxy)-8-((triisopropylsilyl)ethynyl)naphthalen-1-yl)-2-(((S)-1-(3-(4-methoxy-4-oxobutoxy)propyl)pyrrolidin-2-yl)methoxy)pyrido[4,3-d]pyrimidin-4-yl)-3,8-diazabicyclo[3.2.1]octane-8-carboxylate

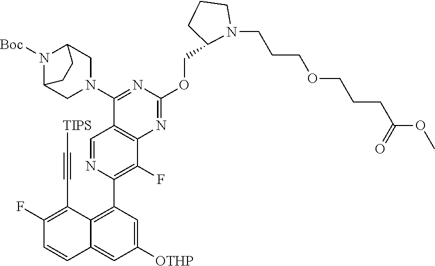

To a solution of methyl methyl (S)-4-(3-(2-(hydroxymethyl)pyrrolidin-1-yl)propoxy)butanoate (57 mg, 0.22 mmol), tert-butyl 3-(8-fluoro-7-(7-fluoro-3-((tetrahydro-2H-pyran-2-yl)oxy)-8-((triisopropylsilyl)ethynyl)naphthalen-1-yl)-2-(methylsulfonyl)pyrido[4,3-d]pyrimidin-4-yl)-3,8-diazabicyclo[3.2.1]octane-8-carboxylate (150 mg, 0.18 mmol) in acetonitrile (1 mL) was added 1,4-diazabicyclo[2.2.2]octane (2 mg, 0.02 mmol) and cesium carbonate (178 mg, 0.55 mmol). The mixture was stirred at 50° C. for 1 h. The reaction mixture was filtered to remove the insoluable byproduct. The filtrate was diluted with water (5 mL) and extracted with ethyl acetate (10 mL×3). The combined organic layers were washed with brine (20 mL), dried over sodium sulfate, filtered and concentrated under reduced pressure to give a residue. The residue was purified by prep-HPLC (column: Phenomenex luna C18 150*40 mm*15 um; mobile phase: [water(FA)-ACN]; B %: 40%-70%, 10 min) to afford the desired product tert-butyl 3-(8-fluoro-7-(7-fluoro-3-((tetrahydro-2H-pyran-2-yl)oxy)-8-((triisopropylsilyl)ethynyl)naphthalen-1-yl)-2-(((S)-1-(3-(4-methoxy-4-oxobutoxy)propyl)pyrrolidin-2-yl)methoxy)pyrido [4,3-d]pyrimidin-4-yl)-3,8-diazabicyclo[3.2.1]octane-8-carboxylate (160 mg, 0.16 mmol, 87% yield) as a white solid. LCMS (ESI, m/z): 1001.4 [M+H]⁺.

Step 7: 4-(3-((2S)-2-(((4-(8-(tert-butoxycarbonyl)-3,8-diazabicyclo[3.2.1]octan-3-yl)-8-fluoro-7-(7-fluoro-3-((tetrahydro-2H-pyran-2-yl)oxy)-8-((triisopropylsilyl)ethynyl)naphthalen-1-yl)pyrido[4,3-d]pyrimidin-2-yl)oxy)methyl)pyrrolidin-1-yl)propoxy)butanoic acid

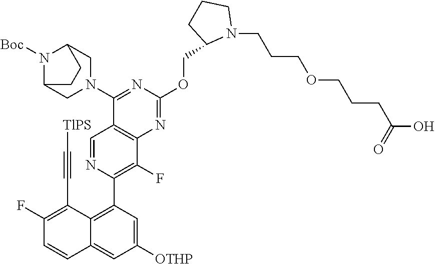

To a solution of tert-butyl 3-(8-fluoro-7-(7-fluoro-3-((tetrahydro-2H-pyran-2-yl)oxy)-8-((triisopropylsilyl)ethynyl)naphthalen-1-yl)-2-(((S)-1-(3-(4-methoxy-4-oxobutoxy)propyl)pyrrolidin-2-yl)methoxy)pyrido[4,3-d]pyrimidin-4-yl)-3,8-diazabicyclo[3.2.1]octane-8-carboxylate (159 mg, 0.16 mmol) in methanol (1.6 mL), tetrahydrofuran (1.6 mL) was added lithium hydroxide (2 M, 0.8 mL). The mixture was stirred at 30° C. for 1 h. The reaction mixture was adjusted pH to 5 by adding aqueous hydrochloride (1 M), and then diluted with ethyl acetate (10 mL), extracted with ethyl acetate (10 mL×3). The combined organic layers were washed with brine (20 mL), dried over sodium sulfate, filtered and concentrated under reduced pressure to afford the crude product 4-(3-((2S)-2-(((4-(8-(tert-butoxycarbonyl)-3,8-diazabicyclo[3.2.1]octan-3-yl)-8-fluoro-7-(7-fluoro-3-((tetrahydro-2H-pyran-2-yl)oxy)-8-((triisopropylsilyl)ethynyl)naphthalen-1-yl)pyrido[4,3-d]pyrimidin-2-yl)oxy)methyl)pyrrolidin-1-yl)propoxy)butanoic acid (150 mg, 0.15 mmol, 95% yield) as a white solid. LCMS (ESI, m/z): 986.7 [M−H]⁺.

Step 8: 4-(3-((2S)-2-(((4-(8-(tert-butoxycarbonyl)-3,8-diazabicyclo[3.2.1]octan-3-yl)-7-(8-ethynyl-7-fluoro-3-((tetrahydro-2H-pyran-2-yl)oxy)naphthalen-1-yl)-8-fluoropyrido[4,3-d]pyrimidin-2-yl)oxy)methyl)pyrrolidin-1-yl)propoxy)butanoic acid

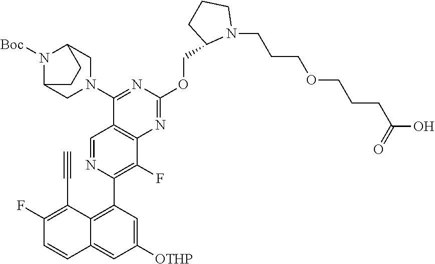

To a solution of 4-(3-((2S)-2-(((4-(8-(tert-butoxycarbonyl)-3,8-diazabicyclo[3.2.1]octan-3-yl)-8-fluoro-7-(7-fluoro-3-((tetrahydro-2H-pyran-2-yl)oxy)-8-((triisopropylsilyl)ethynyl)naphthalen-1-yl)pyrido[4,3-d]pyrimidin-2-yl)oxy)methyl)pyrrolidin-1-yl)propoxy)butanoic acid (150 mg, 0.15 mmol) in N,N-dimethylformamide (2 mL) was added cesium fluoride (230 mg, 1.52 mmol). The mixture was stirred at 25° C. for 1 h. The reaction was filtered to remove the insoluble byproduct. The filtrate was diluted with water (20 mL) and extracted with ethyl acetate (20 mL×3). The combined organic layers were washed with brine (30 mL), dried over sodium sulfate, filtered and concentrated under reduced pressure to afford the crude product 4-(3-((2S)-2-(((4-(8-(tert-butoxycarbonyl)-3,8-diazabicyclo[3.2.1]octan-3-yl)-7-(8-ethynyl-7-fluoro-3-((tetrahydro-2H-pyran-2-yl)oxy)naphthalen-1-yl)-8-fluoropyrido[4,3-d]pyrimidin-2-yl)oxy)methyl)pyrrolidin-1-yl)propoxy)butanoic acid (120 mg, 0.14 mmol, 95% yield) as a yellow solid. LCMS (ESI, m/z): 831.5 [M+H]⁺.

Step 9: tert-butyl 3-(2-(((S)-1-(3-(2-(((S)-1-((2S,4R)-2-(((S)-1-(2′,6′-difluoro-[1,1′-biphenyl]-4-yl)ethyl)carbamoyl)-4-hydroxypyrrolidin-1-yl)-3,3-dimethyl-1-oxobutan-2-yl)amino)-2-oxoethoxy)propyl)pyrrolidin-2-yl)methoxy)-7-(8-ethynyl-7-fluoro-3-((tetrahydro-2H-pyran-2-yl)oxy)naphthalen-1-yl)-8-fluoropyrido[4,3-d]pyrimidin-4-yl)-3,8-diazabicyclo[3.2.1]octane-8-carboxylate

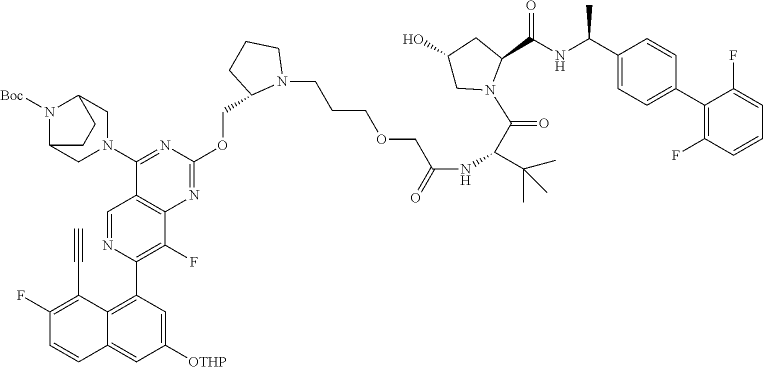

To a solution of 4-(3-((2S)-2-(((4-(8-(tert-butoxycarbonyl)-3,8-diazabicyclo[3.2.1]octan-3-yl)-7-(8-ethynyl-7-fluoro-3-((tetrahydro-2H-pyran-2-yl)oxy)naphthalen-1-yl)-8-fluoropyrido[4,3-d]pyrimidin-2-yl)oxy)methyl)pyrrolidin-1-yl)propoxy)butanoic acid (120 mg, 0.14 mmol), (2S,4R)-1-((S)-2-amino-3,3-dimethylbutanoyl)-N—((S)-1-(2′,6′-difluoro-[1,1′-biphenyl]-4-yl)ethyl)-4-hydroxypyrrolidine-2-carboxamide (99 mg, 0.17 mmol, trifluoroacetate) in dimethylformamide (2 mL) was added diisopropylethylamine (74 mg, 0.57 mmol, 0.1 mL), 1-(3-dimethylaminopropyl)-3-ethylcarbodiimide hydrochloride (83 mg, 0.43 mmol) and 1-hydroxybenzotriazole (58 mg, 0.43 mmol). The mixture was stirred at 25° C. for 16 h. The reaction mixture was quenched by adding water (10 mL), and then extracted with ethyl acetate (10 mL×3). The combined organic layers were washed with brine (20 mL), dried over sodium sulfate, filtered and concentrated under reduced pressure to give a residue. The residue was purified by prep-TLC (9% methanol in dichloromethane) to afford the desired product tert-butyl 3-(2-(((S)-1-(3-(2-(((S)-1-((2S,4R)-2-(((S)-1-(2′,6′-difluoro-[1,1′-biphenyl]-4-yl)ethyl)carbamoyl)-4-hydroxypyrrolidin-1-yl)-3,3-dimethyl-1-oxobutan-2-yl)amino)-2-oxoethoxy)propyl)pyrrolidin-2-yl)methoxy)-7-(8-ethynyl-7-fluoro-3-((tetrahydro-2H-pyran-2-yl)oxy)naphthalen-1-yl)-8-fluoropyrido[4,3-d]pyrimidin-4-yl)-3,8-diazabicyclo[3.2.1]octane-8-carboxylate (100 mg, 0.08 mmol, 54% yield) was obtained as a yellow solid. LCMS (ESI, m/z): 1272.4 [M]⁺.

Step 10: (2S,4R)-1-((2S)-2-(4-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)butanamido)-3,3-dimethylbutanoyl)-N—((S)-1-(2′,6′-difluoro-[1,1′-biphenyl]-4-yl)ethyl)-4-hydroxypyrrolidine-2-carboxamide (Example 34)

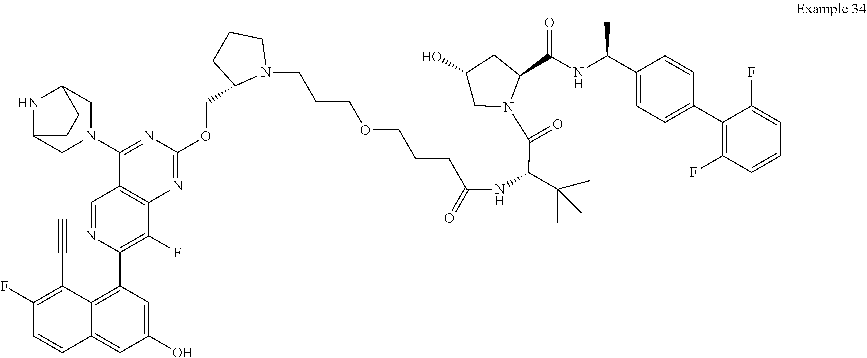 [SMILES: C#Cc1c(F)ccc2cc(O)cc(-c3ncc4c(N5CC6CCC(C5)N6)nc(OC[C@@H]5CCCN5CCCOCCCC(=O)N[C@H](C(=O)N5C[C@H](O)C[C@H]5C(=O)N[C@@H](C)c5ccc(-c6c(F)cccc6F)cc5)C(C)(C)C)nc4c3F)c12]

To a solution of tert-butyl 3-(2-(((S)-1-(3-(2-(((S)-1-((2S,4R)-2-(((S)-1-(2′,6′-difluoro-[1,1′-biphenyl]-4-yl)ethyl)carbamoyl)-4-hydroxypyrrolidin-1-yl)-3,3-dimethyl-1-oxobutan-2-yl)amino)-2-oxoethoxy)propyl)pyrrolidin-2-yl)methoxy)-7-(8-ethynyl-7-fluoro-3-((tetrahydro-2H-pyran-2-yl)oxy)naphthalen-1-yl)-8-fluoropyrido[4,3-d]pyrimidin-4-yl)-3,8-diazabicyclo[3.2.1]octane-8-carboxylate (100 mg, 0.08 mmol) in dichloromethane (1 mL) was added trifluoroacetic acid (1.5 g, 13.55 mmol, 1 mL). The mixture was stirred at 25° C. for 0.5 h. The reaction mixture was concentrated under reduced pressure to remove solvent. The residue was purified by prep-HPLC (column: Unisil 3-100 C18 Ultra 150*50 mm*3 um; mobile phase: [water(FA)-ACN]; B %: 12%-42%, 7 min) to afford the desired product (2S,4R)-1-((2S)-2-(4-(3-((2S)-2-(((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)methyl)pyrrolidin-1-yl)propoxy)butanamido)-3,3-dimethylbutanoyl)-N—((S)-1-(2′,6′-difluoro-[1,1′-biphenyl]-4-yl)ethyl)-4-hydroxypyrrolidine-2-carboxamide (26.72 mg, 0.02 mmol, 29% yield, formate[1]) as a yellow solid. LCMS (ESI, m/z): 1128.5 [M]⁺. ¹H NMR (400 MHz, DMSO) δ 9.05 (s, 1H), 8.38 (d, J=7.6 Hz, 1H), 8.22 (s, 1H), 8.01-7.95 (m, 1H), 7.85-7.78 (m, 1H), 7.51-7.44 (m, 2H), 7.43-7.34 (m, 6H), 7.26-7.17 (m, 3H), 4.99-4.89 (m, 1H), 4.55-4.47 (m, 2H), 4.46-4.39 (m, 2H), 4.36-4.32 (m, 1H), 4.31-4.26 (m, 1H), 4.15-4.05 (m, 1H), 3.96-3.91 (m, 1H), 3.73-3.57 (m, 8H), 3.40-3.27 (m, 6H), 3.09-3.03 (m, 1H), 2.97-2.87 (m, 1H), 2.86-2.79 (m, 1H), 2.44-2.35 (m, 2H), 2.32-2.09 (m, 4H), 2.07-1.98 (m, 1H), 1.97-1.86 (m, 1H), 1.84-1.77 (m, 1H), 1.71-1.59 (m, 7H), 1.39 (br d, J=7.2 Hz, 3H), 0.92 (s, 9H).

Example 35): (2S,4R)-1-((2S)-2-(3-((1-(2-((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)ethyl)piperidin-4-yl)methoxy)isoxazol-5-yl)-3-methylbutanoyl)-4-hydroxy-N—((S)-1-(4-(4-methylthiazol-5-yl)phenyl)ethyl)pyrrolidine-2-carboxamide

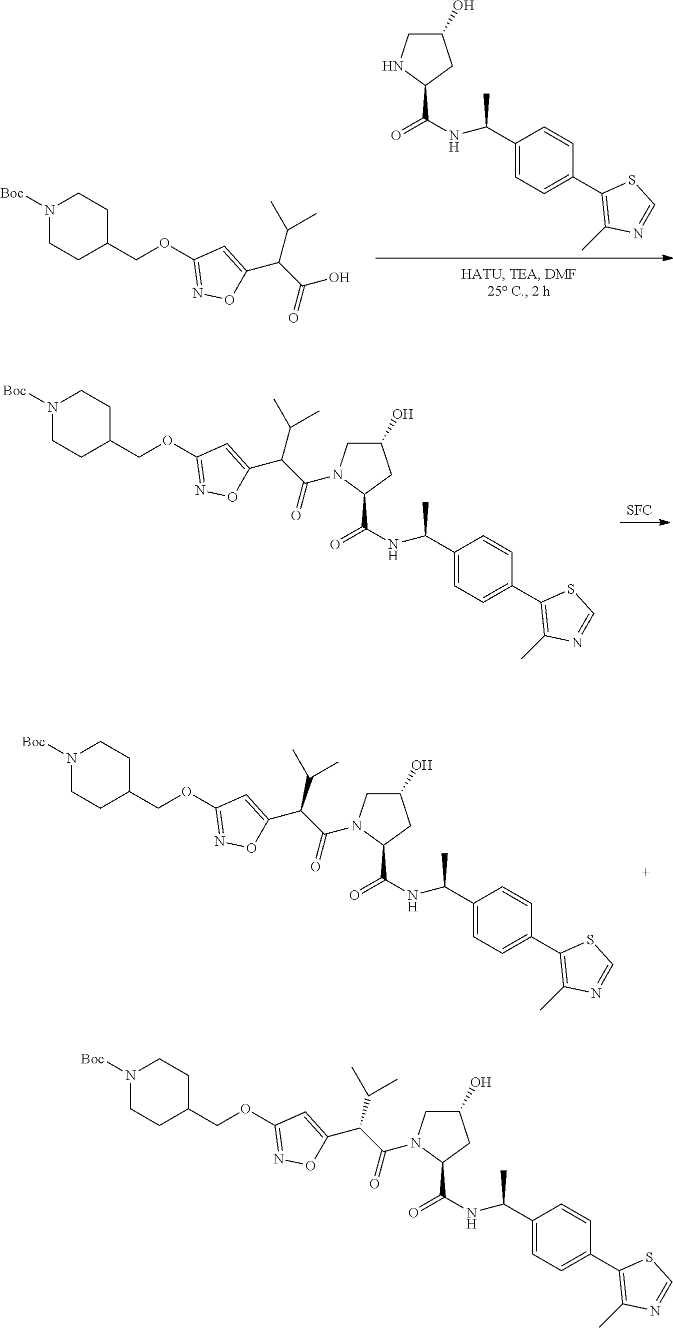 [SMILES: CC(C)C(C(=O)O)c1cc(OCC2CCN(C(=O)OC(C)(C)C)CC2)no1 ; Cc1ncsc1-c1ccc([C@H](C)NC(=O)[C@@H]2C[C@@H](O)CN2)cc1 ; Cc1ncsc1-c1ccc([C@H](C)NC(=O)[C@@H]2C[C@@H](O)CN2C(=O)C(c2cc(OCC3CCN(C(=O)OC(C)(C)C)CC3)no2)C(C)C)cc1 ; "SFC" ; Cc1ncsc1-c1ccc([C@H](C)NC(=O)[C@@H]2C[C@@H](O)CN2C(=O)[C@@H](c2cc(OCC3CCN(C(=O)OC(C)(C)C)CC3)no2)C(C)C)cc1 ; Cc1ncsc1-c1ccc([C@H](C)NC(=O)[C@@H]2C[C@@H](O)CN2C(=O)[C@H](c2cc(OCC3CCN(C(=O)OC(C)(C)C)CC3)no2)C(C)C)cc1]

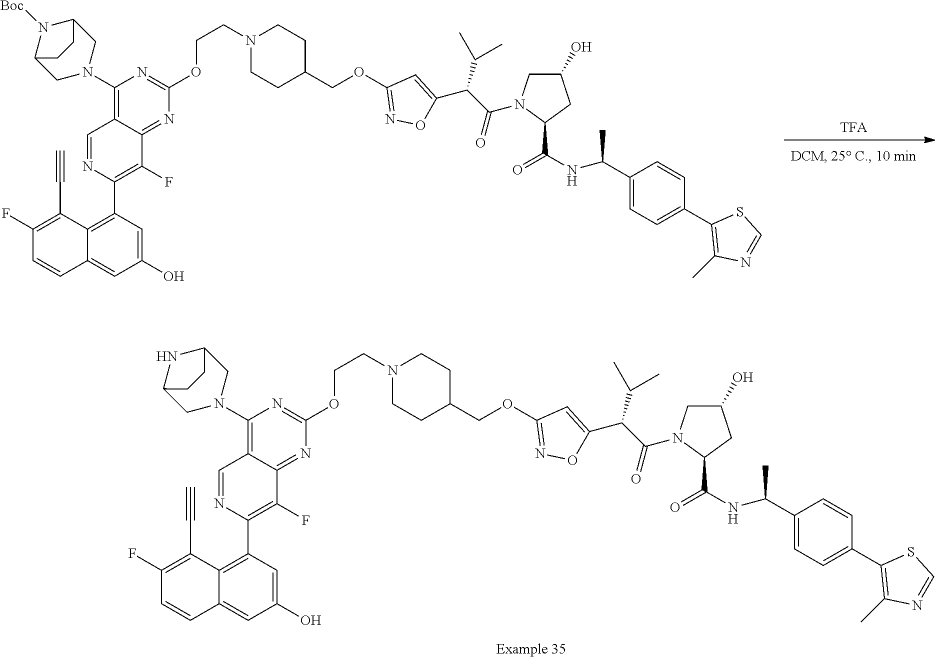 [SMILES: C#Cc1c(F)ccc2cc(O)cc(-c3ncc4c(N5CC6CCC(C5)N6C(=O)OC(C)(C)C)nc(OCCN5CCC(COc6cc([C@@H](C(=O)N7C[C@H](O)C[C@H]7C(=O)N[C@@H](C)c7ccc(-c8scnc8C)cc7)C(C)C)on6)CC5)nc4c3F)c12 ; "TFA" ; C#Cc1c(F)ccc2cc(O)cc(-c3ncc4c(N5CC6CCC(C5)N6)nc(OCCN5CCC(COc6cc([C@@H](C(=O)N7C[C@H](O)C[C@H]7C(=O)N[C@@H](C)c7ccc(-c8scnc8C)cc7)C(C)C)on6)CC5)nc4c3F)c12]

Step 1: tert-butyl 4-(((5-(1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)methyl)piperidine-1-carboxylate.

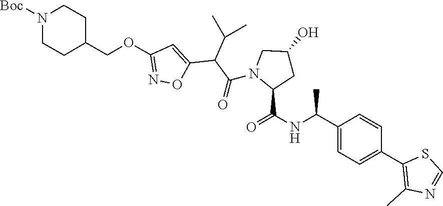 [SMILES: Cc1ncsc1-c1ccc([C@H](C)NC(=O)[C@@H]2C[C@@H](O)CN2C(=O)C(c2cc(OCC3CCN(C(=O)OC(C)(C)C)CC3)no2)C(C)C)cc1]

To a solution of 2-[3-[(1-tert-butoxycarbonyl-4-piperidyl)methoxy]isoxazol-5-yl]-3-methyl-butanoic acid (500 mg, 1.31 mmol, 1.00 eq) and (2S,4R)-4-hydroxy-N-[(1S)-1-[4-(4-methylthiazol-5-yl)phenyl]ethyl]pyrrolidine-2-carboxamide (433 mg, 1.31 mmol, 1 eq) in N,N-dimethylformamide (8 mL) was added diisopropylethylamine (507 mg, 3.92 mmol, 3.00 eq) and o-(7-azabenzotriazol-1-yl)-n,n,n′,n′-tetramethyluronium hexafluorophosphate (646 mg, 1.70 mmol, 1.30 eq). The mixture was stirred at 25° C. for 1 h. LCMS showed desired mass was detected. The mixture was filtered and the filtrate was purified by prep-HPLC (column: Phenomenex luna C18 150*40 mm*15 um; mobile phase: [water(FA)-ACN]; B %: 40%-70%, 10 min) to afford the product (440 mg, 0.62 mmol, 47% yield, 98% purity) as a yellow solid. LCMS (ESI, m/z): 696.2 [M+1]⁺.

Step 2: tert-butyl 4-(((5-((S)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)methyl)piperidine-1-carboxylate.

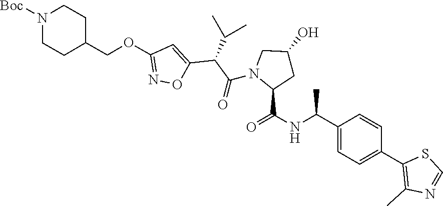 [SMILES: Cc1ncsc1-c1ccc([C@H](C)NC(=O)[C@@H]2C[C@@H](O)CN2C(=O)[C@H](c2cc(OCC3CCN(C(=O)OC(C)(C)C)CC3)no2)C(C)C)cc1]

The mixture of diastereoisomers tert-butyl4-[[5-[1-[(2S,4R)-4-hydroxy-2-[[(1S)-1-[4-(4-methylthiazol-5-yl)phenyl]ethyl] carbamoyl]pyrrolidine-1-carbonyl]-2-methyl-propyl]isoxazol-3-yl]oxymethyl] piperidine-1-carboxylate (400 mg, 0.57 mmol, 1.00 eq) was separated by SFC (column: DAICEL CHIRALPAK AD (250 mm*30 mm, 10 um); mobile phase: [0.1% NH3H2O IPA]; B %: 40%-40%, 3.8 min), and the second eluent (t_{R}=2.076 min) was identified as the desired diastereoisomer tert-butyl 4-(((5-((S)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)methyl)piperidine-1-carboxylate (170 mg, 0.24 mmol, 42% yield, 99% purity) as white solid.

Step 3: (2S,4R)-4-hydroxy-1-((S)-3-methyl-2-(3-(piperidin-4-ylmethoxy)isoxazol-5-yl)butanoyl)-N—((S)-1-(4-(4-methylthiazol-5-yl)phenyl)ethyl)pyrrolidine-2-carboxamide.

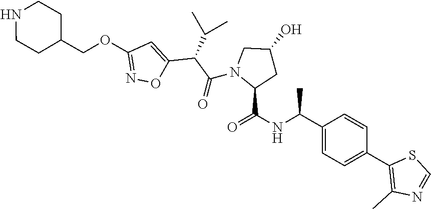 [SMILES: Cc1ncsc1-c1ccc([C@H](C)NC(=O)[C@@H]2C[C@@H](O)CN2C(=O)[C@H](c2cc(OCC3CCNCC3)no2)C(C)C)cc1]

To a solution of tert-butyl 4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl] piperidine-1-carboxylate (120 mg, 0.17 mmol, 1.00 eq) in dichloromethane (2 mL), was added hydrogen chloride/dioxane (4 M, 2 mL, 46.39 eq). The mixture was stirred at 30° C. for 0.5 h. TLC (dichloromethane/methanol=10/1) showed that the reaction was completed. The reaction mixture was concentrated under reduced pressure to get the crude product (109 mg, 0.17 mmol, 99% yield, hydrochloride) as a white solid, which was used in the next step directly.

Step 4: tert-butyl 3-(8-fluoro-7-(7-fluoro-3-hydroxy-8-((triisopropylsilyl)ethynyl)naphthalen-1-yl)-2-(2-(4-(((5-((S)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)methyl)piperidin-1-yl)ethoxy)pyrido[4,3-d]pyrimidin-4-yl)-3,8-diazabicyclo[3.2.1]octane-8-carboxylate.

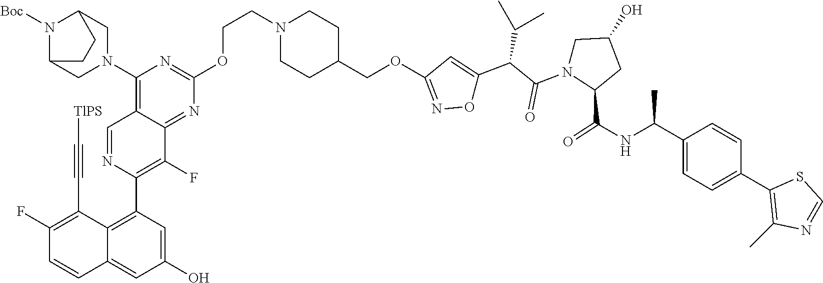

To a solution of (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl] pyrrolidine-2-carboxamide (130 mg, 0.2 mmol, 1 eq, hydrochloride) in the mixed solvent of dichloromethane (2 mL) and dimethylsulfoxide (1 mL) was added diisopropylethylamine (53 mg, 0.41 mmol, 2 eq). Then tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (92 mg, 0.12 mmol, 0.6 eq), acetic acid (24 mg, 0.41 mmol, 2 eq) and sodium triacetoxyborohydride (65 mg, 0.3 mmol, 1.5 eq) were added. The mixture was stirred at 25° C. for 0.5 h. LCMS showed the reaction was completed. Water (30 mL) was added before the reaction mixture was extracted with ethyl acetate (30 mL×3). The combined organic phase was washed with brine (30 mL×2), dried over anhydrous anhydrous sodium sulfate, filtered and concentrated in vacuum. The residue was purified by prep-TLC (Dichloromethane/Methanol=10/1) to afford the product (130 mg, 0.097 mmol, 47% yield) as a white solid. LCMS (ESI, m/z): 1137.8 [M]⁺.

Step 5: tert-butyl 3-(7-(8-ethynyl-7-fluoro-3-hydroxynaphthalen-1-yl)-8-fluoro-2-(2-(4-(((5-((S)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)methyl)piperidin-1-yl)ethoxy)pyrido[4,3-d]pyrimidin-4-yl)-3,8-diazabicyclo[3.2.1]octane-8-carboxylate.

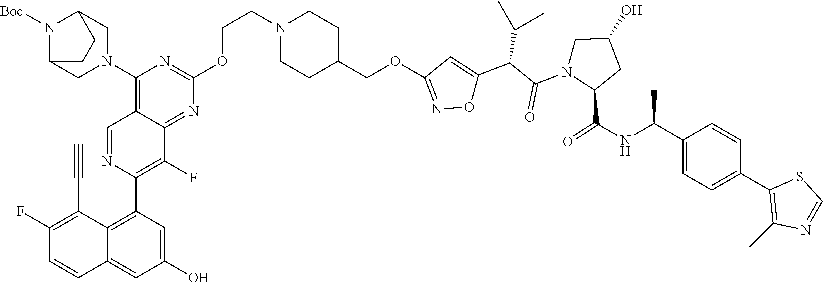 [SMILES: C#Cc1c(F)ccc2cc(O)cc(-c3ncc4c(N5CC6CCC(C5)N6C(=O)OC(C)(C)C)nc(OCCN5CCC(COc6cc([C@@H](C(=O)N7C[C@H](O)C[C@H]7C(=O)N[C@@H](C)c7ccc(-c8scnc8C)cc7)C(C)C)on6)CC5)nc4c3F)c12]

To a solution of tert-butyl 3-[8-fluoro-7-[7-fluoro-3-hydroxy-8-(2-triisopropylsilylethynyl)-1-naphthyl]-2-[2-[4-[[5-[(1R)-1-[(2S,4R)-4-hydroxy-2-[[(1S)-1-[4-(4-methylthiazol-5-yl)phenyl] ethyl]carbamoyl]pyrrolidine-1-carbonyl]-2-methyl-propyl] isoxazol-3-yl]oxymethyl]-1-piperidyl]ethoxy]pyrido[4,3-d] pyrimidin-4-yl]-3,8-diazabicyclo[3.2.1]octane-8-carboxylate (65 mg, 0.048 mmol, 1 eq) in N,N-dimethylformamide (2 mL) was added cesium fluoride (110 mg, 0.72 mmol, 15 eq). The mixture was stirred at 25° C. for 1 h. LCMS showed the reaction was completed. The reaction mixture was quenched by adding saturated aqueous ammonium chloride (10 mL), then the mixture was extracted with dichloromethane (30 mL×3). The combined organic layers were washed with brine (30 mL×2), dried over anhydrous sodium sulfate, filtered and concentrated in vacuum to get the product (57 mg, 0.48 mmol, 99% yield) as a white solid, which was used into next step without further purification. LCMS (ESI, m/z): 1181.6 [M]⁺.

Step 6: (2S,4R)-1-((2R)-2-(3-((1-(2-((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)ethyl)piperidin-4-yl)methoxy)isoxazol-5-yl)-3-methylbutanoyl)-4-hydroxy-N—((S)-1-(4-(4-methylthiazol-5-yl)phenyl)ethyl)pyrrolidine-2-carboxamide (Example 35).

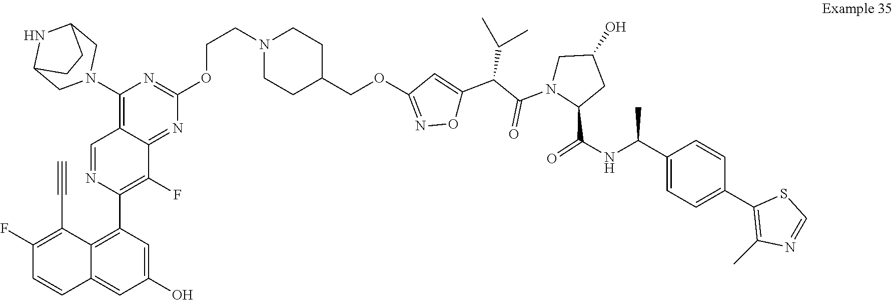 [SMILES: C#Cc1c(F)ccc2cc(O)cc(-c3ncc4c(N5CC6CCC(C5)N6)nc(OCCN5CCC(COc6cc([C@@H](C(=O)N7C[C@H](O)C[C@H]7C(=O)N[C@@H](C)c7ccc(-c8scnc8C)cc7)C(C)C)on6)CC5)nc4c3F)c12]

To a solution of tert-butyl 3-[7-(8-ethynyl-7-fluoro-3-hydroxy-1-naphthyl)-8-fluoro-2-[2-[4-[[5-[(1R)-1-[(2S,4R)-4-hydroxy-2-[[(1S)-1-[4-(4-methylthiazol-5-yl)phenyl]ethyl]carbamoyl] pyrrolidine-1-carbonyl]-2-methyl-propyl]isoxazol-3-yl]oxymethyl]-1-piperidyl]ethoxy]pyrido [4,3-d]pyrimidin-4-yl]-3,8-diazabicyclo[3.2.1]octane-8-carboxylate (57 mg, 0.048 mmol, 1 eq) in dichloromethane (2 mL) was added triethylamine (1.54 g, 13.51 mmol, 1 mL, 279.92 eq). The mixture was stirred at 25° C. for 10 min. LCMS showed the reaction was completed. The reaction mixture was concentrated in reduced pressure to give a residue. The residue was purified by prep-HPLC (column: Phenomenex Synergi C18 150*25 mm*10 um; mobile phase: [water(FA)-ACN]; B %: 8%-38%, 9 min) to afford the desired product (10.2 mg, 0.006 mmol, 14% yield, formate[1]) as a yellow solid. LCMS (ESI, m/z): 1081.5 [M]⁺, ¹H NMR (400 MHz, DMSO-d₆) δ: 9.03 (s, 1H), 9.00-8.95 (m, 1H), 8.30-8.18 (m, 2H), 7.96 (dd, J=6.4, 8.9 Hz, 1H), 7.50-7.33 (m, 5H), 7.32-7.29 (m, 1H), 7.17 (d, J=2.4 Hz, 1H), 6.12-6.03 (m, 1H), 4.93-4.84 (m, 1H), 4.53-4.24 (m, 7H), 4.00-3.92 (m, 3H), 3.68-3.58 (m, 5H), 3.02-2.87 (m, 3H), 2.74-2.63 (m, 3H), 2.45-2.43 (m, 3H), 2.31-2.16 (m, 2H), 2.11-1.84 (m, 4H), 1.79 (br d, J=2.8 Hz, 7H), 1.45 (d, J=7.2 Hz, 1H), 1.38-1.30 (m, 3H), 1.28-1.19 (m, 2H), 0.95 (d, J=6.8 Hz, 3H), 0.86-0.79 (m, 3H).

Example 36: (2S,4R)-1-((2R)-2-(3-(7-(2-((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)ethyl)-2,7-diazaspiro[3.5]nonan-2-yl)isoxazol-5-yl)-3-methylbutanoyl)-4-hydroxy-N—((S)-1-(2′,4′,6′-trifluoro-[1,1′-biphenyl]-4-yl)ethyl)pyrrolidine-2-carboxamide

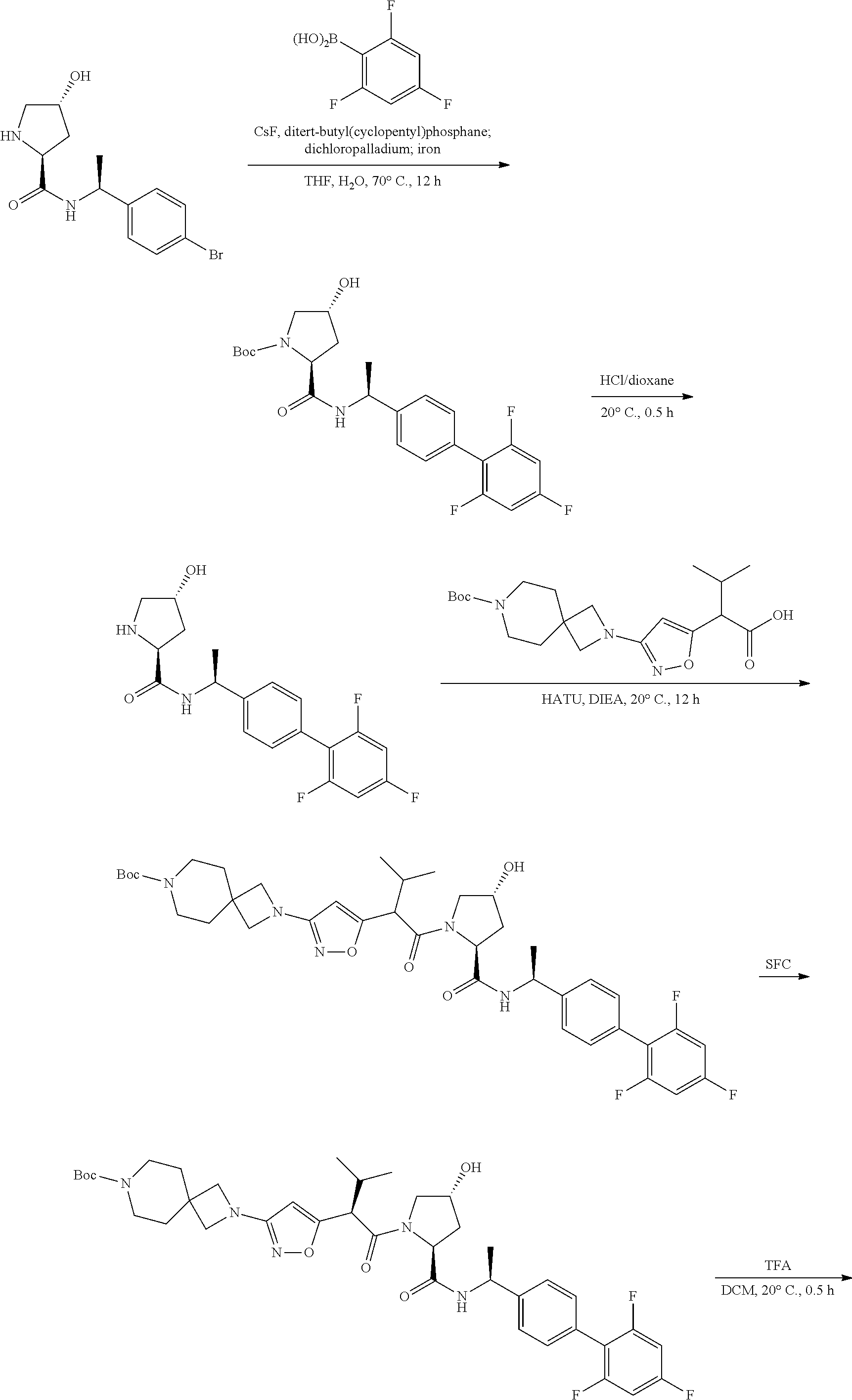

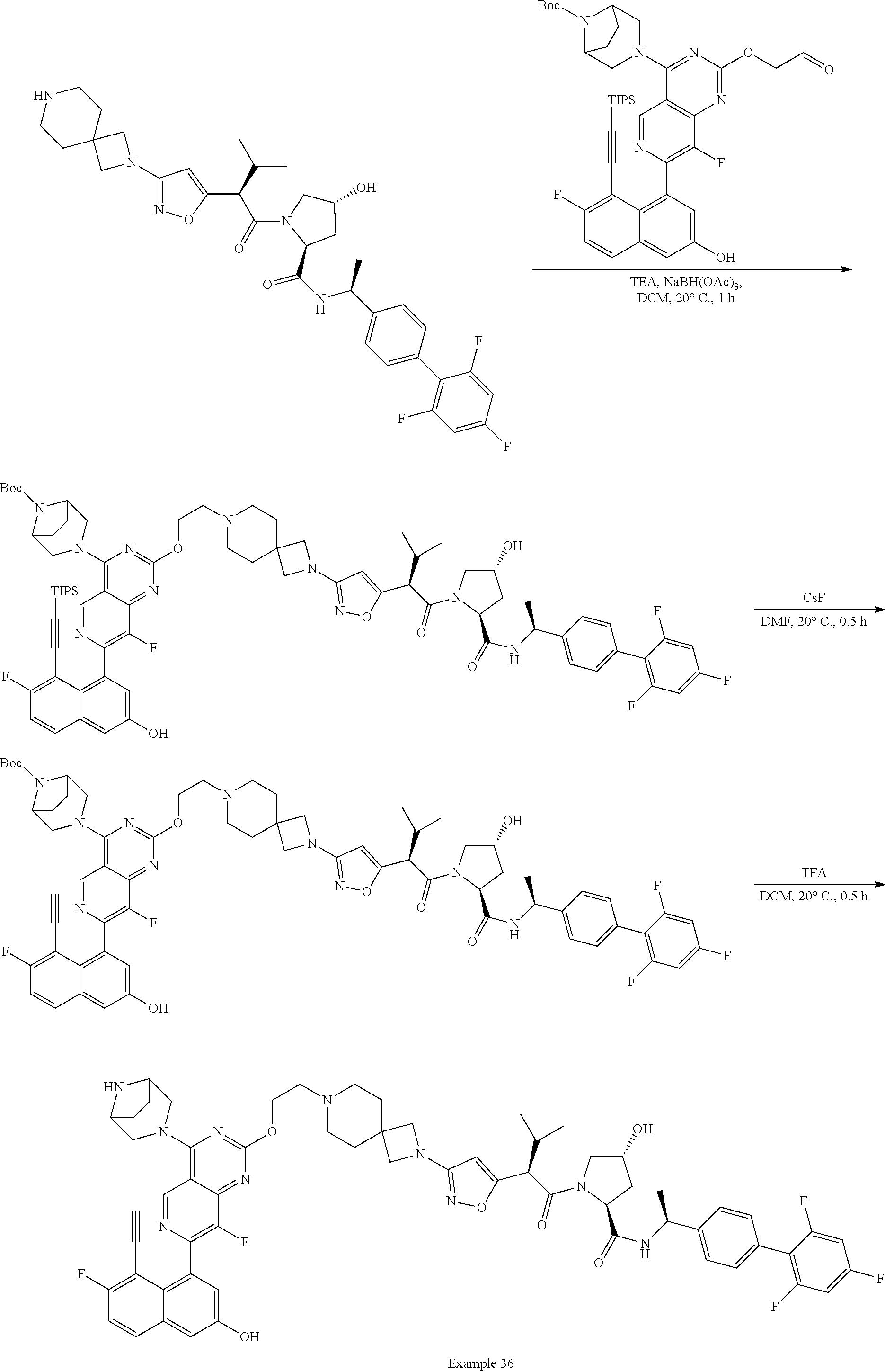

Step 1: tert-butyl (2S,4R)-4-hydroxy-2-(((S)-1-(2′,4′,6′-trifluoro-[1,1′-biphenyl]-4-yl)ethyl)carbamoyl)pyrrolidine-1-carboxylate

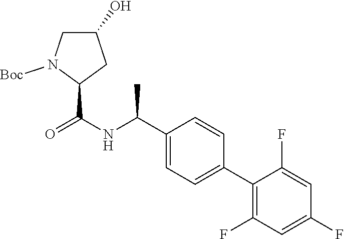 [SMILES: C[C@H](NC(=O)[C@@H]1C[C@@H](O)CN1C(=O)OC(C)(C)C)c1ccc(-c2c(F)cc(F)cc2F)cc1]

A mixture of (2S,4R)—N—((S)-1-(4-bromophenyl)ethyl)-4-hydroxypyrrolidine-2-carboxamide (5.00 g, 12.10 mmol, 1.00 eq), (2,4,6-trifluorophenyl)boronic acid (6.38 g, 36.29 mmol, 3.00 eq), cesium fluoride (5.51 g, 36.29 mmol, 3.00 eq), ditert-butyl(cyclopentyl)phosphane; dichloropalladium; iron (1.58 g, 2.42 mmol, 0.20 eq) in tetrahydrofuran (80 mL) and water (10 mL) was degassed and purged with nitrogen for 3 times. The mixture was stirred at 70° C. for 12 h under nitrogen atmosphere. The mixture was diluted with water (200 mL) and extracted with ethyl acetate (100 mL×3). The combined organic layers were washed with brine (200 mL), dried over anhydrous sodium sulfate filtered and concentrated under reduced pressure to give a residue. The residue was purified by preparative high liquid chromatography (column: Phenomenex luna C18 250*80 mm*10 um; mobile phase: [water(FA)-ACN]; B %: 45%-75%, 20 min) to give tert-butyl (2S,4R)-4-hydroxy-2-(((S)-1-(2′,4′,6′-trifluoro-[1,1′-biphenyl]-4-yl)ethyl)carbamoyl)pyrrolidine-1-carboxylate (2.60 g, 5.54 mmol, 46% yield, 99% purity) as an off-white solid. LCMS (ESI, m/z): 465.2 [M+H]⁺. 1H NMR: (400 MHz, DMSO-d₆) δ 8.44-8.32 (m, 1H), 7.46-7.23 (m, 6H), 5.04-4.95 (m, 1H), 4.29-4.17 (m, 2H), 3.47-3.34 (m, 1H), 3.33-3.24 (m, 1H), 2.14-2.03 (m, 1H), 1.83-1.74 (m, 1H), 1.44-1.30 (m, 12H).

Step 2: (S)—N—((S)-1-(2′,4′,6′-trifluoro-[1,1′-biphenyl]-4-yl)ethyl)pyrrolidine-2-carboxamide

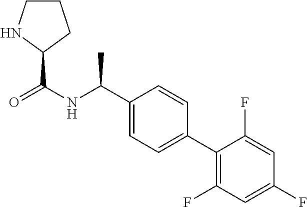 [SMILES: C[C@H](NC(=O)[C@@H]1CCCN1)c1ccc(-c2c(F)cc(F)cc2F)cc1]

To a solution of tert-butyl (2S,4R)-4-hydroxy-2-(((S)-1-(2′,4′,6′-trifluoro-[1,1′-biphenyl]-4-yl)ethyl)carbamoyl)pyrrolidine-1-carboxylate (2.10 g, 4.52 mmol, 1.00 eq) in dichloromethane (10 mL) was added hydrochloric acid/dioxane (4 M, 8 mL, 7.08 eq). The mixture was stirred at 20° C. for 0.5 h. The mixture was concentrated under reduced pressure to give compound (S)—N—((S)-1-(2′,4′,6′-trifluoro-[1,1′-biphenyl]-4-yl)ethyl)pyrrolidine-2-carboxamide (1.70 g, 4.07 mmol, 90% yield, 96% purity, hydrochloride) as a white solid. LCMS (ESI, m/z): 365.3 [M+H]⁺.

Step 3: tert-butyl 2-(5-(1-((2S,4R)-4-hydroxy-2-(((S)-1-(2′,4′,6′-trifluoro-[1,1′-biphenyl]-4-yl)ethyl)carbamoyl)pyrrolidin-1-yl)-3-methyl-1-oxobutan-2-yl)isoxazol-3-yl)-2,7-diazaspiro[3.5]nonane-7-carboxylate

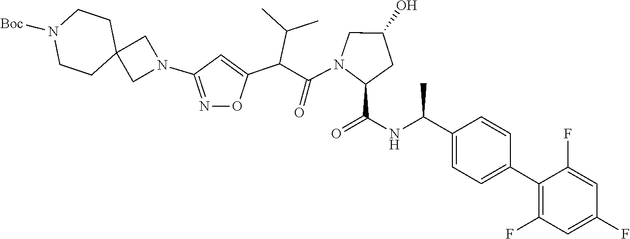 [SMILES: CC(C)C(C(=O)N1C[C@H](O)C[C@H]1C(=O)N[C@@H](C)c1ccc(-c2c(F)cc(F)cc2F)cc1)c1cc(N2CC3(CCN(C(=O)OC(C)(C)C)CC3)C2)no1]

To a solution of 2-[3-(7-tert-butoxycarbonyl-2,7-diazaspiro[3.5]nonan-2-yl)isoxazol-5-yl]-3-methyl-butanoic acid (1.06 g, 2.69 mmol, 1.20 eq) and (S)—N—((S)-1-(2′,4′,6′-trifluoro-[1,1′-biphenyl]-4-yl)ethyl)pyrrolidine-2-carboxamide (900 mg, 2.25 mmol, 1.00 eq, hydrochloride) in N,N-dimethylformamide (15 mL) was added O-(7-azabenzotriazol-1-yl)-N,N,N,N-tetramethyluronium hexafluorophosphate (1.28 g, 3.37 mmol, 1.50 eq) and diisopropylethylamine (1.60 g, 12.35 mmol, 2 mL, 5.50 eq). The mixture was stirred at 20° C. for 12 h. The mixture was diluted with water (100 mL) and extracted with ethyl acetate (50 mL×2). The combined organic layers were washed with brine (100 mL), dried over anhydrous sodium sulfate filtered and concentrated under reduced pressure to give a residue. The residue was purified by preparative high liquid chromatography (column: Phenomenex luna C18 250*80 mm*10 um; mobile phase: [water(FA)-ACN]; B %: 50%-80%, 20 min) to give tert-butyl 2-(5-(1-((2S,4R)-4-hydroxy-2-(((S)-1-(2′,4′,6′-trifluoro-[1,1′-biphenyl]-4-yl)ethyl)carbamoyl)pyrrolidin-1-yl)-3-methyl-1-oxobutan-2-yl)isoxazol-3-yl)-2,7-diazaspiro[3.5]nonane-7-carboxylate (940 mg, 1.27 mmol, 57% yield) as a brown solid. LCMS: (ESI, m/z): 740.3 [M+H]⁺. 1H NMR: (400 MHz, DMSO-d6) δ 8.46-8.09 (m, 1H), 7.63-7.04 (m, 6H), 5.90-5.70 (m, 1H), 5.18-4.18 (m, 4H), 3.80-3.06 (m, 12H), 2.43-1.43 (m, 8H), 1.39 (s, 9H), 1.32-0.65 (m, 7H).

Step 4: tert-butyl 2-(5-((R)-1-((2S,4R)-4-hydroxy-2-(((S)-1-(2′,4′,6′-trifluoro-[1,1′-biphenyl]-4-yl)ethyl)carbamoyl)pyrrolidin-1-yl)-3-methyl-1-oxobutan-2-yl)isoxazol-3-yl)-2,7-diazaspiro[3.5]nonane-7-carboxylate.

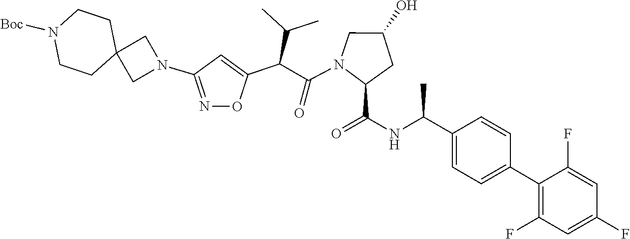 [SMILES: CC(C)[C@@H](C(=O)N1C[C@H](O)C[C@H]1C(=O)N[C@@H](C)c1ccc(-c2c(F)cc(F)cc2F)cc1)c1cc(N2CC3(CCN(C(=O)OC(C)(C)C)CC3)C2)no1]

The mixture of diastereoisomers tert-butyl 2-(5-(1-((2S,4R)-4-hydroxy-2-(((S)-1-(2′,4′,6′-trifluoro-[1,1′-biphenyl]-4-yl)ethyl)carbamoyl)pyrrolidin-1-yl)-3-methyl-1-oxobutan-2-yl)isoxazol-3-yl)-2,7-diazaspiro[3.5]nonane-7-carboxylate (940 mg, 1.27 mmol, 1.00 eq) was separated by SFC (column: Phenomenex luna C18 250*80 mm*10 um; mobile phase: [water(FA)-ACN]; B %: 50%-80%, 20 min), the second eluent was collected and identified as the desired diastereoisomer tert-butyl 2-(5-((R)-1-((2S,4R)-4-hydroxy-2-(((S)-1-(2′,4′,6′-trifluoro-[1,1′-biphenyl]-4-yl)ethyl)carbamoyl)pyrrolidin-1-yl)-3-methyl-1-oxobutan-2-yl)isoxazol-3-yl)-2,7-diazaspiro[3.5]nonane-7-carboxylate (400 mg, 0.54 mmol, 24% yield) as a white solid.

Step 5: (2S,4R)-1-((R)-2-(3-(2,7-diazaspiro[3.5]nonan-2-yl)isoxazol-5-yl)-3-methylbutanoyl)-4-hydroxy-N—((S)-1-(2′,4′,6′-trifluoro-[1,1′-biphenyl]-4-yl)ethyl)pyrrolidine-2-carboxamide

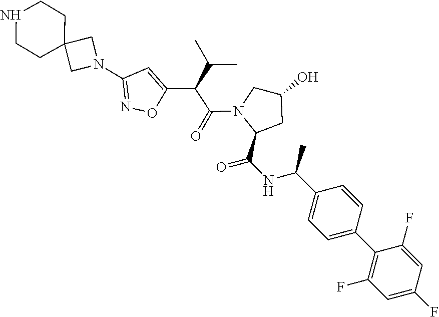 [SMILES: CC(C)[C@@H](C(=O)N1C[C@H](O)C[C@H]1C(=O)N[C@@H](C)c1ccc(-c2c(F)cc(F)cc2F)cc1)c1cc(N2CC3(CCNCC3)C2)no1]

To a solution of tert-butyl 2-(5-((R)-1-((2S,4R)-4-hydroxy-2-(((S)-1-(2′,4′,6′-trifluoro-[1,1′-biphenyl]-4-yl)ethyl)carbamoyl)pyrrolidin-1-yl)-3-methyl-1-oxobutan-2-yl)isoxazol-3-yl)-2,7-diazaspiro[3.5]nonane-7-carboxylate (400 mg, 0.54 mmol, 1.00 eq) in dichloromethane (10 mL) was added trifluoroacetic acid (3.00 g, 27.01 mmol, 2 mL, 37.01 eq). The mixture was stirred at 20° C. for 0.5 h. The mixture was concentrated under reduced pressure to give compound (2S,4R)-1-((R)-2-(3-(2,7-diazaspiro[3.5]nonan-2-yl)isoxazol-5-yl)-3-methylbutanoyl)-4-hydroxy-N—((S)-1-(2′,4′,6′-trifluoro-[1,1′-biphenyl]-4-yl)ethyl)pyrrolidine-2-carboxamide (320 mg, 0.50 mmol, 93% yield) as a brown solid. LCMS: (ESI, m/z): 640.5 [M+H]⁺.

Step 6: tert-butyl 3-(8-fluoro-7-(7-fluoro-3-hydroxy-8-((triisopropylsilyl)ethynyl)naphthalen-1-yl)-2-(2-(2-(5-((R)-1-((2S,4R)-4-hydroxy-2-(((S)-1-(2′,4′,6′-trifluoro-[1,1′-biphenyl]-4-yl)ethyl)carbamoyl)pyrrolidin-1-yl)-3-methyl-1-oxobutan-2-yl)isoxazol-3-yl)-2,7-diazaspiro[3.5]nonan-7-yl)ethoxy)pyrido[4,3-d]pyrimidin-4-yl)-3,8-diazabicyclo[3.2.1]octane-8-carboxylate

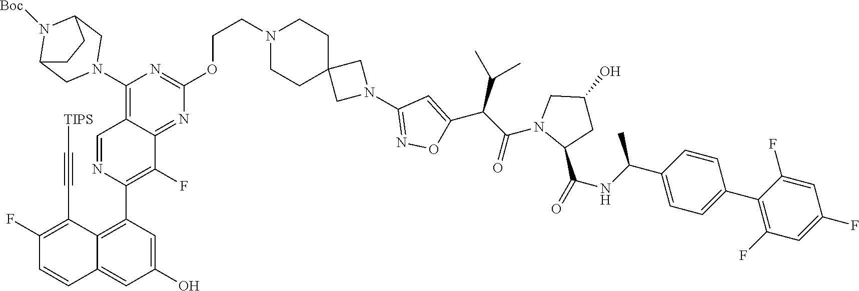

To a solution of (2S,4R)-1-((R)-2-(3-(2,7-diazaspiro[3.5]nonan-2-yl)isoxazol-5-yl)-3-methylbutanoyl)-4-hydroxy-N—((S)-1-(2′,4′,6′-trifluoro-[1,1′-biphenyl]-4-yl)ethyl)pyrrolidine-2-carboxamide (100 mg, 0.13 mmol, 1.00 eq, trifluoroacetate) in dichloromethane (1 mL) and dimethylsulfoxide (1 mL) was added diisopropylethylamine (51 mg, 0.40 mmol, 3.00 eq) at 20° C., then tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.I]octane-8-carboxylate (70 mg, 0.09 mmol, 0.70 eq) was added to the mixture, and the mixture was stirred at 20° C. for 0.5 h. Sodium triacetoxyborohydride (70 mg, 0.33 mmol, 2.50 eq) was added to the mixture and the mixture was stirred at 20° C. for 0.5 h. The mixture was diluted with water (30 mL) and extracted with ethyl acetate (50 mL×3). The combined organic layers were washed with brine (100 mL), dried over anhydrous sodium sulfate filtered and concentrated under reduced pressure to give a residue. The residue was purified by preparative thin layer chromatography (dichloromethane/methanol=10/1, R_{f}=0.45) to give tert-butyl 3-(8-fluoro-7-(7-fluoro-3-hydroxy-8-((triisopropylsilyl)ethynyl)naphthalen-1-yl)-2-(2-(2-(5-((R)-1-((2S,4R)-4-hydroxy-2-(((S)-1-(2′,4′,6′-trifluoro-[1,1′-biphenyl]-4-yl)ethyl)carbamoyl)pyrrolidin-1-yl)-3-methyl-1-oxobutan-2-yl)isoxazol-3-yl)-2,7-diazaspiro[3.5]nonan-7-yl)ethoxy)pyrido[4,3-d]pyrimidin-4-yl)-3,8-diazabicyclo[3.2.1]octane-8-carboxylate (50 mg, 0.036 mmol, 27% yield, 100% purity) as a white solid. LCMS: (ESI, m/z): 1381.3 [M]⁺.

Step 7: tert-butyl 3-(7-(8-ethynyl-7-fluoro-3-hydroxynaphthalen-1-yl)-8-fluoro-2-(2-(2-(5-((R)-1-((2S,4R)-4-hydroxy-2-(((S)-1-(2′,4′,6′-trifluoro-[1,1′-biphenyl]-4-yl)ethyl)carbamoyl)pyrrolidin-1-yl)-3-methyl-1-oxobutan-2-yl)isoxazol-3-yl)-2,7-diazaspiro[3.5]nonan-7-yl)ethoxy)pyrido[4,3-d]pyrimidin-4-yl)-3,8-diazabicyclo[3.2.1]octane-8-carboxylate

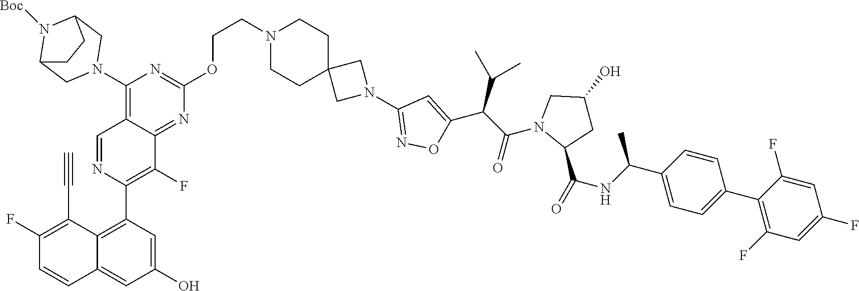 [SMILES: C#Cc1c(F)ccc2cc(O)cc(-c3ncc4c(N5CC6CCC(C5)N6C(=O)OC(C)(C)C)nc(OCCN5CCC6(CC5)CN(c5cc([C@H](C(=O)N7C[C@H](O)C[C@H]7C(=O)N[C@@H](C)c7ccc(-c8c(F)cc(F)cc8F)cc7)C(C)C)on5)C6)nc4c3F)c12]

To a solution of tert-butyl 3-(8-fluoro-7-(7-fluoro-3-hydroxy-8-((triisopropylsilyl)ethynyl)naphthalen-1-yl)-2-(2-(2-(5-((R)-1-((2S,4R)-4-hydroxy-2-(((S)-1-(2′,4′,6′-trifluoro-[1,1′-biphenyl]-4-yl)ethyl)carbamoyl)pyrrolidin-1-yl)-3-methyl-1-oxobutan-2-yl)isoxazol-3-yl)-2,7-diazaspiro[3.5]nonan-7-yl)ethoxy)pyrido[4,3-d]pyrimidin-4-yl)-3,8-diazabicyclo[3.2.1]octane-8-carboxylate (40 mg, 0.03 mmol, 1.00 eq) in N,N-dimethylformamide (2 mL) was added cesium fluoride (66 mg, 0.43 mmol, 15.00 eq) at 20° C. and the mixture was stirred at 20° C. for 0.5 h. The mixture was diluted with water (40 mL) and extracted with ethyl acetate (20 mL×2). The combined organic layers were washed with brine (40 mL), dried over anhydrous sodium sulfate filtered and concentrated under reduced pressure to give compound tert-butyl 3-(7-(8-ethynyl-7-fluoro-3-hydroxynaphthalen-1-yl)-8-fluoro-2-(2-(2-(5-((R)-1-((2S,4R)-4-hydroxy-2-(((S)-1-(2′,4′,6′-trifluoro-[1,1′-biphenyl]-4-yl)ethyl)carbamoyl)pyrrolidin-1-yl)-3-methyl-1-oxobutan-2-yl)isoxazol-3-yl)-2,7-diazaspiro[3.5]nonan-7-yl)ethoxy)pyrido[4,3-d]pyrimidin-4-yl)-3,8-diazabicyclo[3.2.1]octane-8-carboxylate (30 mg, 0.02 mmol, 85% yield) as a brown solid. LCMS (ESI, m/z): 1225.2 [M]⁺.

Step 8: (2S,4R)-1-((2R)-2-(3-(7-(2-((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)ethyl)-2,7-diazaspiro[3.5]nonan-2-yl)isoxazol-5-yl)-3-methylbutanoyl)-4-hydroxy-N—((S)-1-(2′,4′,6′-trifluoro-[1,1′-biphenyl]-4-yl)ethyl)pyrrolidine-2-carboxamide (Example 36)

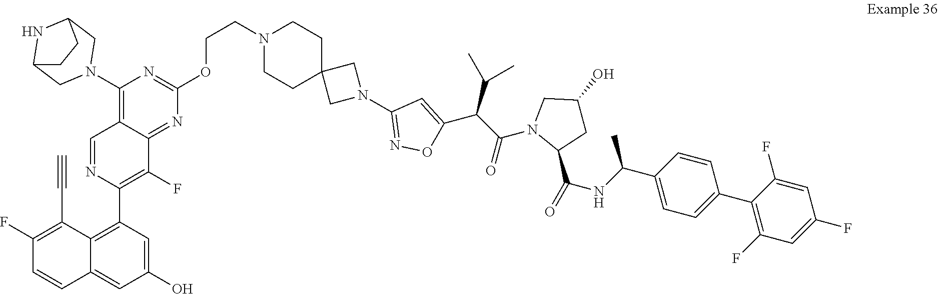 [SMILES: C#Cc1c(F)ccc2cc(O)cc(-c3ncc4c(N5CC6CCC(C5)N6)nc(OCCN5CCC6(CC5)CN(c5cc([C@H](C(=O)N7C[C@H](O)C[C@H]7C(=O)N[C@@H](C)c7ccc(-c8c(F)cc(F)cc8F)cc7)C(C)C)on5)C6)nc4c3F)c12]

To a solution of tert-butyl 3-(7-(8-ethynyl-7-fluoro-3-hydroxynaphthalen-1-yl)-8-fluoro-2-(2-(2-(5-((R)-1-((2S,4R)-4-hydroxy-2-(((S)-1-(2′,4′,6′-trifluoro-[1,1′-biphenyl]-4-yl)ethyl)carbamoyl)pyrrolidin-1-yl)-3-methyl-1-oxobutan-2-yl)isoxazol-3-yl)-2,7-diazaspiro[3.5]nonan-7-yl)ethoxy)pyrido[4,3-d]pyrimidin-4-yl)-3,8-diazabicyclo[3.2.1]octane-8-carboxylate (30 mg, 0.02 mmol, 1.00 eq) in dichloromethane (5 mL) was added trifluoroacetic acid (7.00 g, 61.39 mmol, 5.0 mL, 2507.50 eq). The mixture was stirred at 20° C. for 0.5 h. The mixture was concentrated to give a residue. The residue was purified by preparative high liquid chromatography (column: Phenomenex Synergi C18 150*25 mm*10 um; mobile phase: [water(FA)-ACN]; B %: 20%-40%, 10 min) to give compound (2S,4R)-1-((2R)-2-(3-(7-(2-((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)ethyl)-2,7-diazaspiro[3.5]nonan-2-yl)isoxazol-5-yl)-3-methylbutanoyl)-4-hydroxy-N—((S)-1-(2′,4′,6′-trifluoro-[1,1′-biphenyl]-4-yl)ethyl)pyrrolidine-2-carboxamide (12.27 mg, 0.011 mmol, 45% yield, 100% purity) as a yellow solid. LCMS (ESI, m/z): 1125.6 [M]+. ¹H NMR: (400 MHz, DMSO-d₆) δ 9.04 (s, 1H), 8.42 (d, J=7.6 Hz, 1H), 8.02-7.91 (m, 1H), 7.49-7.42 (m, 1H), 7.41-7.29 (m, 7H), 7.18 (d, J=2.4 Hz, 1H), 5.83 (s, 1H), 5.28-4.80 (m, 2H), 4.52-4.25 (m, 6H), 3.94 (s, 1H), 3.73-3.62 (m, 2H), 3.60-3.55 (m, 7H), 3.41 (d, J=12.0 Hz, 2H), 2.72-2.63 (m, 2H), 2.39-2.30 (m, 2H), 2.26-2.11 (m, 2H), 2.11-2.09 (m, 1H), 2.06-1.94 (m, 1H), 1.82-1.65 (m, 9H), 1.46 (d, J=6.8 Hz, 1H), 1.39 (d, J=7.2 Hz, 3H), 0.99-0.90 (m, 3H), 0.82-0.73 (m, 3H).

Example 37: Synthesis of (2S,4R)-1-((2S)-2-(3-(3-((2S)-2-(((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)pyrrolidin-1-yl)propoxy)propanamido)-3,3-dimethylbutanoyl)-4-hydroxy-N—((S)-1-(2′,4′,6′-trifluoro-[1,1′-biphenyl]-4-yl)ethyl)pyrrolidine-2-carboxamide

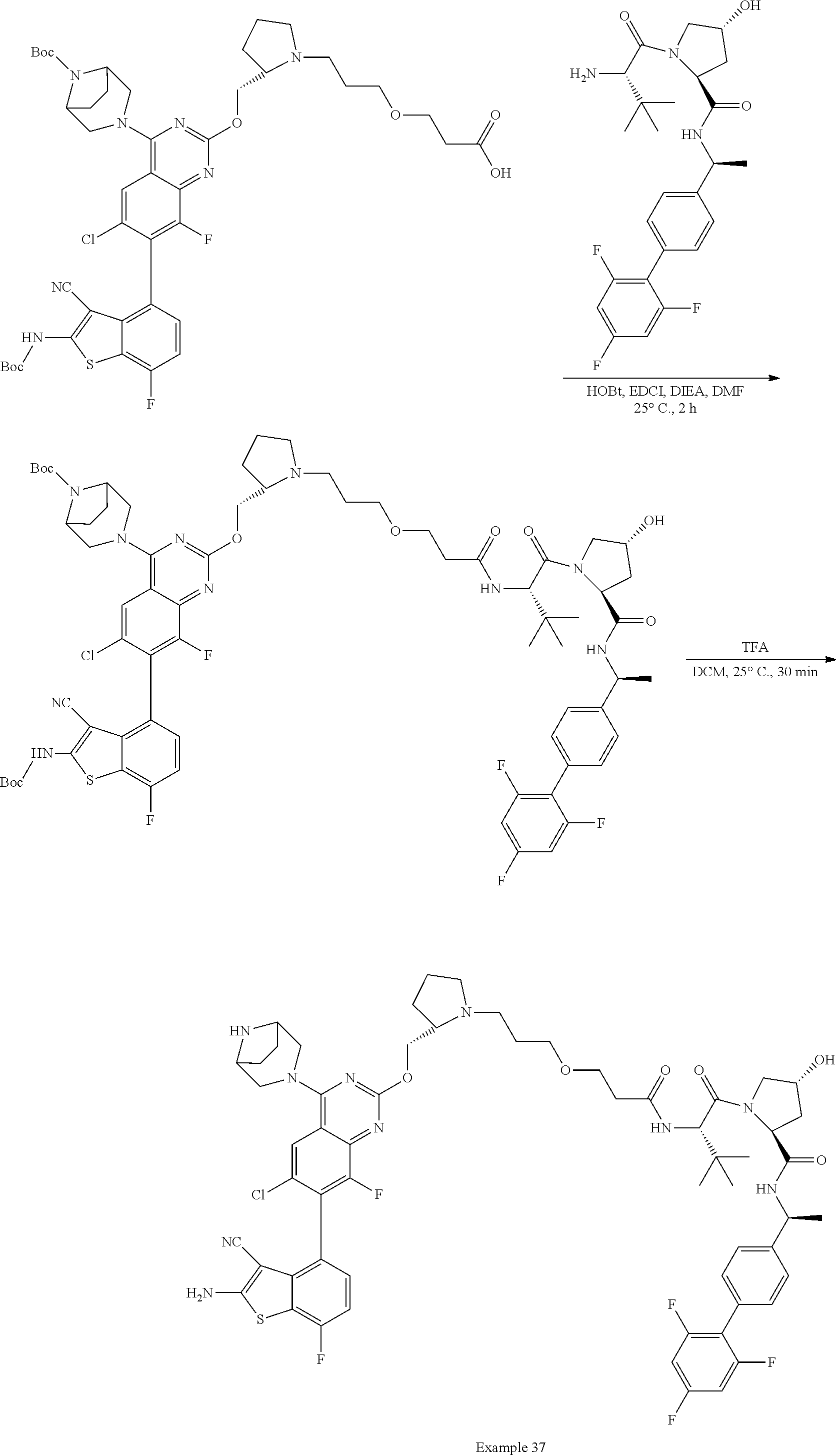

Step 1: tert-butyl 3-(7-(2-((tert-butoxycarbonyl)amino)-3-cyano-7-fluorobenzo[b]thiophen-4-yl)-6-chloro-8-fluoro-2-(((S)-1-(3-(3-(((S)-1-((2S,4R)-4-hydroxy-2-(((S)-1-(2′,4′,6′-trifluoro-[1,1′-biphenyl]-4-yl)ethyl)carbamoyl)pyrrolidin-1-yl)-3,3-dimethyl-1-oxobutan-2-yl)amino)-3-oxopropoxy)propyl)pyrrolidin-2-yl)methoxy)quinazolin-4-yl)-3,8-diazabicyclo[3.2.1]octane-8-carboxylate

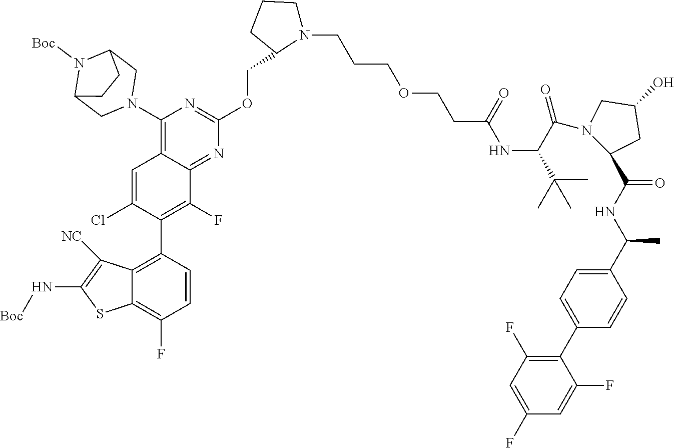

To a solution of 3-(3-((2S)-2-(((4-(8-(tert-butoxycarbonyl)-3,8-diazabicyclo[3.2.1]octan-3-yl)-7-(2-((tert-butoxycarbonyl)amino)-3-cyano-7-fluorobenzo[b]thiophen-4-yl)-6-chloro-8-fluoroquinazolin-2-yl)oxy)methyl) pyrrolidin-1-yl)propoxy)propanoic acid and (2S,4R)-1-((S)-2-amino-3,3-dimethylbutanoyl)-4-hydroxy-N—((S)-1-(2′,4′,6′-trifluoro-[1,1′-biphenyl]-4-yl)ethyl)pyrrolidine-2-carboxamide (140 mg, 0.23 mmol, 1.2 eq, TFA) in N,N-dimethylformamide (2 mL) was added 1-hydroxybenzotriazole (79 mg, 0.59 mmol, 3 eq), diisopropylethylamine (127 mg, 0.98 mmol, 5 eq) and 1-ethyl-3-(3-dimethylaminopropyl)carbodiimide hydrochloride (113 mg, 0.59 mmol, 3 eq). The mixture was stirred at 25° C. for 2 h. The reaction mixture was extracted with ethyl acetate (20 mL×3). The combined organic phase was washed with brine (20 mL×2), dried over anhydrous sodium sulfate, filtered and concentrated in vacuum to get a residue. The residue was purified by prep-TLC (9% methanol in dichloromethane) to afford the desired product tert-butyl 3-(7-(2-((tert-butoxycarbonyl)amino)-3-cyano-7-fluorobenzo[b]thiophen-4-yl)-6-chloro-8-fluoro-2-(((S)-1-(3-(3-(((S)-1-((2S,4R)-4-hydroxy-2-(((S)-1-(2′,4′,6′-trifluoro-[1,1′-biphenyl]-4-yl)ethyl)carbamoyl)pyrrolidin-1-yl)-3,3-dimethyl-1-oxobutan-2-yl)amino)-3-oxopropoxy)propyl)pyrrolidin-2-yl)methoxy)quinazolin-4-yl)-3,8-diazabicyclo[3.2.1]octane-8-carboxylate (145 mg, 0.10 mmol, 53% yield) as a yellow solid. LCMS (ESI, m/z): 1373.8 [M+H]⁺.

Step 2: (2S,4R)-1-((2S)-2-(3-(3-((2S)-2-(((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)pyrrolidin-1-yl)propoxy)propanamido)-3,3-dimethylbutanoyl)-4-hydroxy-N—((S)-1-(2′,4′,6′-trifluoro-[1,1′-biphenyl]-4-yl)ethyl)pyrrolidine-2-carboxamide (Example 37)

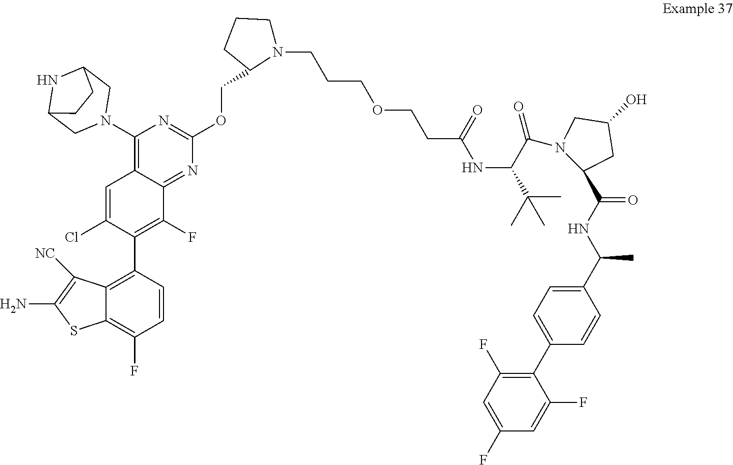

To a solution of tert-butyl 3-(7-(2-((tert-butoxycarbonyl)amino)-3-cyano-7-fluorobenzo[b]thiophen-4-yl)-6-chloro-8-fluoro-2-(((S)-1-(3-(3-(((S)-1-((2S,4R)-4-hydroxy-2-(((S)-1-(2′,4′,6′-trifluoro-[1,1′-biphenyl]-4-yl)ethyl)carbamoyl)pyrrolidin-1-yl)-3,3-dimethyl-1-oxobutan-2-yl)amino)-3-oxopropoxy)propyl)pyrrolidin-2-yl)methoxy)quinazolin-4-yl)-3,8-diazabicyclo[3.2.1]octane-8-carboxylate (145 mg, 0.10 mmol, 1 eq) in dichloromethane (1 mL) was added trifluoroacetic acid (1.54 g, 13.51 mmol, 1 mL, 127 eq). The mixture was stirred at 25° C. for 30 min. The mixture was concentrated in reduced pressure. The residue was purified by prep-HPLC (column: Phenomenex Synergi C18 150*25 mm*10 um; mobile phase: [water(FA)-ACN]; B %: 15%-45%, 10 min) to afford the product (2S,4R)-1-((2S)-2-(3-(3-((2S)-2-(((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)pyrrolidin-1-yl)propoxy)propanamido)-3,3-dimethylbutanoyl)-4-hydroxy-N—((S)-1-(2′,4′,6′-trifluoro-[1,1′-biphenyl]-4-yl)ethyl)pyrrolidine-2-carboxamide (89 mg, 0.07 mmol, 71% yield, 100% purity) as a yellow solid. LCMS (ESI, m/z): 1171.73 [M+H]⁺. ¹H NMR (400 MHz, DMSO-d₆) δ 8.37 (br d, J=8.4 Hz, 1H), 8.14 (s, 1H), 8.11 (br s, 1H), 7.88 (s, 1H), 7.83-7.78 (m, 1H), 7.40 (s, 4H), 7.31 (t, J=8.8 Hz, 2H), 7.24 (dd, J=5.4, 8.4 Hz, 1H), 7.15 (t, J=8.8 Hz, 1H), 4.96-4.87 (m, 1H), 4.51 (br d, J=9.6 Hz, 1H), 4.41 (br t, J=8.4 Hz, 2H), 4.35 (br d, J=2.4 Hz, 1H), 4.29 (br d, J=15.6 Hz, 1H), 4.17-4.07 (m, 1H), 3.98-3.86 (m, 2H), 3.77-3.70 (m, 1H), 3.67-3.43 (m, 8H), 3.08 (br dd, J=2.0, 4.4 Hz, 1H), 2.97-2.84 (m, 2H), 2.54 (br s, 3H), 2.31-2.19 (m, 3H), 2.03-1.97 (m, 1H), 1.94-1.87 (m, 1H), 1.84-1.75 (m, 5H), 1.75-1.63 (m, 5H), 1.37 (br d, J=6.8 Hz, 3H), 0.90 (br d, J=3.0 Hz, 9H).

Example 38: (2S,4R)-1-((2S)-2-(4-(3-((2S)-2-(((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)pyrrolidin-1-yl)propoxy)butanamido)-3,3-dimethylbutanoyl)-4-hydroxy-N—((S)-1-(4-(4-methylthiazol-5-yl)phenyl)ethyl)pyrrolidine-2-carboxamide

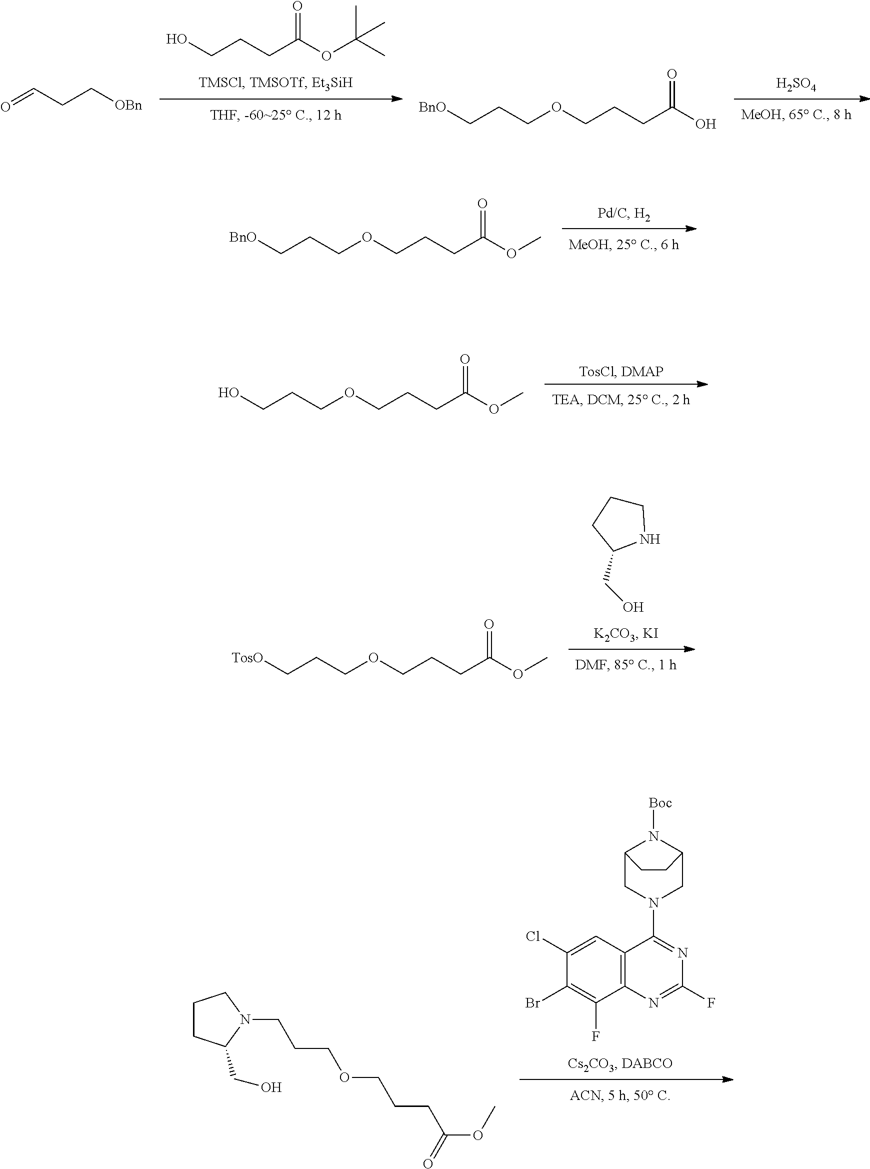

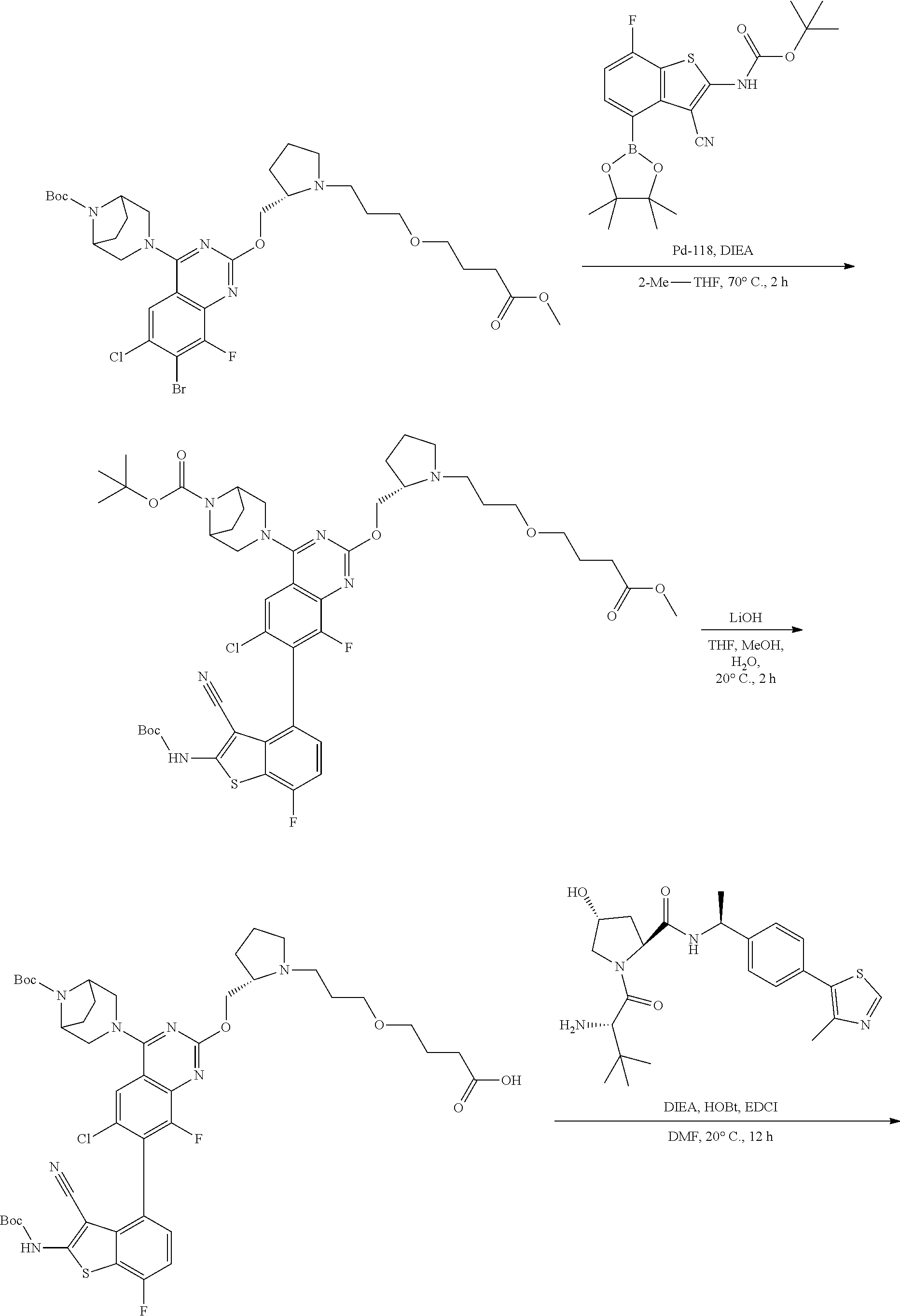

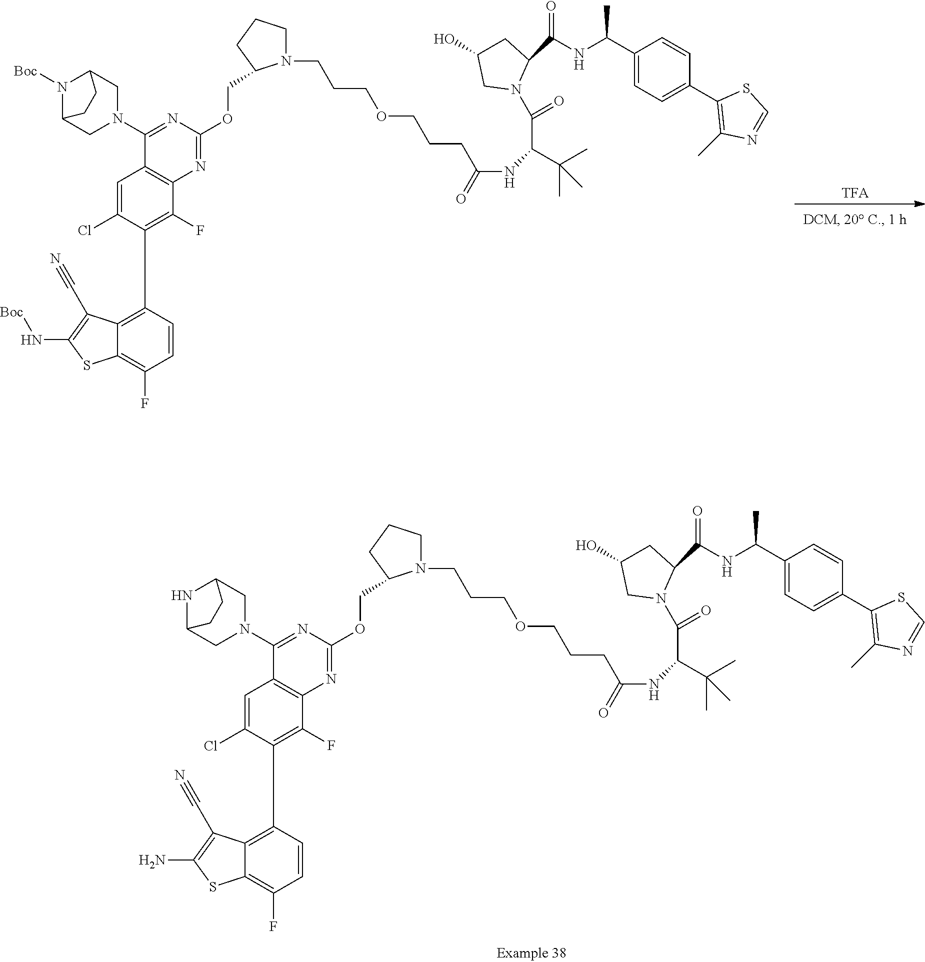 [SMILES: Cc1ncsc1-c1ccc([C@H](C)NC(=O)[C@@H]2C[C@@H](O)CN2C(=O)[C@@H](NC(=O)CCCOCCCN2CCC[C@H]2COc2nc(N3CC4CCC(C3)N4C(=O)OC(C)(C)C)c3cc(Cl)c(-c4ccc(F)c5sc(NC(=O)OC(C)(C)C)c(C#N)c45)c(F)c3n2)C(C)(C)C)cc1 ; "TFA" ; Cc1ncsc1-c1ccc([C@H](C)NC(=O)[C@@H]2C[C@@H](O)CN2C(=O)[C@@H](NC(=O)CCCOCCCN2CCC[C@H]2COc2nc(N3CC4CCC(C3)N4)c3cc(Cl)c(-c4ccc(F)c5sc(N)c(C#N)c45)c(F)c3n2)C(C)(C)C)cc1]

Step 1: 4-(3-(benzyloxy)propoxy)butanoic acid

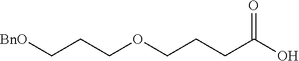 [SMILES: O=C(O)CCCOCCCOCc1ccccc1]

To a solution of tert-butyl 4-hydroxybutanoate (4.9 g, 30.45 mmol, 1 eq) in tetrahydrofuran (20 mL) was added chlorotrimethylsilane (3.6 g, 33.5 mmol, 1.1 eq) and triethylamine (3.54 g, 35. mmol, 4.87 mL, 1.15 eq). The mixture was stirred at 20° C. for 1 h. The reaction mixture was filtered. The filtrate was concentreted under reduced pressure to give the residue. To a solution of 3-(benzyloxy)propanal (5 g, 30 mmol, 1 eq) in dichloromethane (10 mL) was added triethylsilane (4 g, 35 mmol, 1.15 eq) and trimethylsilyl trifluoromethanesulfonate (3.4 g, 15 mmol, 0.5 eq) at −60° C. The mixture was stirred at 20° C. for 11 h. LCMS showed the starting material was consumed completely and one main peak with desired mass was detected. The reaction mixture was quenched by addition sodium hydrogencarbonate (20 mL) at 25° C., and then diluted with ethyl acetate 50 mL and extracted with ethyl acetate (50 mL×3). The combined organic layers were washed with water (50 mL), dried over anhydrous sodium sulfate, filtered and concentrated under reduced pressure to give a residue. The residue was purified by prep-HPLC (column: Phenomenex luna C18 (250*70 mm, 10 um); mobile phase: [water (formic acid)-acetonitrile]; B %: 25%-55%, 20 min) to afford the desired product (3.23 g, 12.80 mmol, 42% yield) as a colorless oil. ¹H NMR (400 MHz, CDCl₃) δ 7.39-7.28 (m, 5H), 4.56-4.48 (m, 2H), 4.36 (t, J=7.2 Hz, 1H), 3.59-3.45 (m, 5H), 2.56-2.40 (m, 2H), 2.33-2.21 (m, 1H), 1.93-1.85 (m, 3H).

Step 2: methyl 4-(3-(benzyloxy)propoxy)butanoate

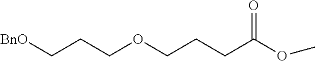 [SMILES: COC(=O)CCCOCCCOCc1ccccc1]

To a solution of 4-(3-(benzyloxy)propoxy)butanoic acid (3.23 g, 12.80 mmol, 1 eq) in methanol (30 mL) was added sulfuric acid (1.26 g, 12.80 mmol, 1 eq). The mixture was stirred at 65° C. for 8 h. LCMS showed the starting material was consumed completely and one main peak with desired mass was detected. The reaction mixture was concentrated under reduced pressure to remove solvent. The residue was diluted with water 10 mL and extracted with ethyl acetate (100 mL×3). The combined organic layers were washed with water (100 mL×3), dried over anhydrous sodium carbonate, filtered. The filtrate was concentrated under reduced pressure to afford the product (3 g, 10.6 mmol, 83% yield) as a colorless oil. LCMS (ESI, m/z): 289.4 [M+Na]⁺.

Step 3: methyl 4-(3-hydroxypropoxy)butanoate

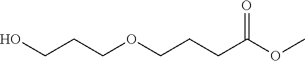

To a solution of methyl 4-(3-(benzyloxy)propoxy)butanoate (2.98 g, 11.2 mmol, 1 eq) in methanol (20 mL) was added palladium on activated carbon catalyst (500 mg, 10% purity) under nitrogen. The suspension was degassed under vacuum and purged with hydrogen several times. The mixture was stirred under hydrogen (15 psi) at 25° C. for 6 h. TLC (dichloromethane/methanol=20/1) indicated the starting material was consumed completely and one new spot formed. The reaction mixture was filtered and the filtrate was concentrated under reduced pressure to afford the product (1.98 g, crude) as a colorless oil. ¹H NMR (400 MHz, CDCl₃) (3.76 (t, J=5.6 Hz, 2H), 3.68 (s, 3H), 3.60 (t, J=5.6 Hz, 2H), 3.47 (t, J=6.4 Hz, 2H), 2.40 (t, J=7.2 Hz, 2H), 2.35-2.12 (m, 1H), 1.95-1.77 (m, 4H).

Step 4: methyl 4-(3-(tosyloxy)propoxy)butanoate

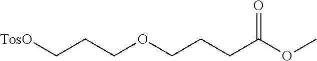

To a solution of methyl 4-(3-hydroxypropoxy)butanoate (1.98 g, 11.24 mmol, 1 eq) in dichloromethane (10 mL) was added 4-methylbenzenesulfonyl chloride (2.57 g, 13.48 mmol, 1.2 eq) and triethylamine (3.41 g, 33.71 mmol, 3 eq). The mixture was stirred at 25° C. for 2 h. LCMS showed the starting material was consumed completely and one main peak with desired mass was detected. The reaction mixture was concentrated under reduced pressure to remove solvent. The residue was purified by column chromatography (silicon dioxide, petroleum ether/ethyl acetate=1/1) to afford the desired product (3 g, 8.8 mmol, 78% yield) as a yellow oil. LCMS (ESI, m/z): 353.0 [M+Na]⁺. ¹H NMR (400 MHz, CDCl₃) (7.80 (d, J=8.4 Hz, 2H), 7.35 (d, J=8.0 Hz, 2H), 4.12 (t, J=6.4 Hz, 2H), 3.67 (s, 3H), 3.47-3.30 (m, 4H), 2.45 (s, 3H), 2.33 (t, J=7.6 Hz, 2H), 1.94-1.75 (m, 4H)

Step 5: methyl (S)-4-(3-(2-(hydroxymethyl)pyrrolidin-1-yl)propoxy)butanoate

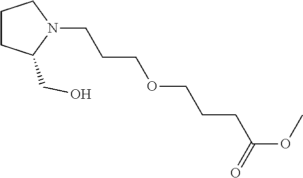 [SMILES: COC(=O)CCCOCCCN1CCC[C@H]1CO]

To a solution of methyl 4-(3-(tosyloxy)propoxy)butanoate (3 g, 9.3 mmol, 1 eq) and (S)-pyrrolidin-2-ylmethanol (1.87 g, 18.5 mmol, 2 eq) in acetonitrile (3 mL) was added potassium iodide (154 mg, 0.9 mmol, 0.1 eq) and potassium carbonate (3.84 g, 28 mmol, 3 eq). The mixture was stirred at 85° C. for 1 h. TLC (Petroleum ether/Ethyl acetate=1/1) indicated the starting material was consumed completely and many new spots formed. The reaction mixture was diluted with water (10 mL) and extracted with ethyl acetate (10 mL×3). The combined organic layers were washed with brine (10 mL×3), dried over anhydrous sodium sulfate, filtered. The filtrate was concentrated under reduced pressure to give a residue. The residue was purified by column chromatography (petroleum ether/ethyl acetate=1/1) to afford the product (1.5 g, 5.8 mmol, 62% yield) as a colorless oil. ¹H NMR (400 MHz, CDCl₃) (3.68 (s, 3H), 3.62 (dd, J=3.6, 10.8 Hz, 1H), 3.53-3.41 (m, 4H), 3.37 (dd, J=2.4, 10.8 Hz, 1H), 3.20-3.13 (m, 1H), 2.85 (td, J=8.0, 12.0 Hz, 1H), 2.58 (td, J=2.8, 5.6 Hz, 1H), 2.41 (t, J=7.6 Hz, 2H), 2.36-2.19 (m, 2H), 1.94-1.68 (m, 8H).

Step 6: tert-butyl 3-(7-bromo-6-chloro-8-fluoro-2-(((S)-1-(3-(4-methoxy-4-oxobutoxy)propyl)pyrrolidin-2-yl)methoxy)quinazolin-4-yl)-3,8-diazabicyclo[3.2.1]octane-8-carboxylate

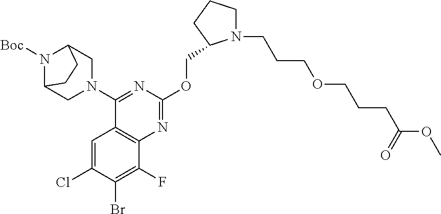 [SMILES: COC(=O)CCCOCCCN1CCC[C@H]1COc1nc(N2CC3CCC(C2)N3C(=O)OC(C)(C)C)c2cc(Cl)c(Br)c(F)c2n1]

To a solution of tert-butyl (1R,5S)-3-(7-bromo-6-chloro-2,8-difluoroquinazolin-4-yl)-3,8-diazabicyclo[3.2.1]octane-8-carboxylate (200 mg, 0.4 mmol, 1 eq) and methyl (S)-4-(3-(2-(hydroxymethyl)pyrrolidin-1-yl)propoxy)butanoate (127 mg, 0.5 mmol, 1.2 eq) in acetonitrile (3 mL) was added cesium carbonate (266 mg, 0.8 mmol, 2 eq) and 1,4-diazabicyclo[2.2.2]octane (4.6 mg, 0.04 mmol, 0.1 eq). The mixture was stirred at 50° C. for 5 h. LCMS showed the starting material was consumed completely and one main peak with desired mass was detected. The reaction mixture was diluted with water (10 mL) and extracted with ethyl acetate (10 mL×3). The combined organic layers were washed with brine (10 mL×3), dried over anhydrous sodium sulfate filtered. The filtrate was concentrated under reduced pressure to give a residue. The residue was purified by prep-TLC (petroleum ether/ethyl acetate=3/1) to afford the product (160 mg, 0.2 mmol, 46% yield) as a yellow solid. LCMS (ESI, m/z): 728.1, 730.3 [M+H]⁺.

Step 7: tert-butyl 3-(7-(2-((tert-butoxycarbonyl)amino)-3-cyano-7-fluorobenzo[b]thiophen-4-yl)-6-chloro-8-fluoro-2-(((S)-1-(3-(4-methoxy-4-oxobutoxy)propyl)pyrrolidin-2-yl)methoxy)quinazolin-4-yl)-3,8-diazabicyclo[3.2.1]octane-8-carboxylate

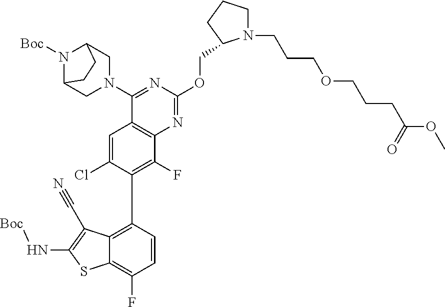 [SMILES: COC(=O)CCCOCCCN1CCC[C@H]1COc1nc(N2CC3CCC(C2)N3C(=O)OC(C)(C)C)c2cc(Cl)c(-c3ccc(F)c4sc(NC(=O)OC(C)(C)C)c(C#N)c34)c(F)c2n1]

A mixture of tert-butyl 3-(7-bromo-6-chloro-8-fluoro-2-(((S)-1-(3-(4-methoxy-4-oxobutoxy)propyl)pyrrolidin-2-yl)methoxy)quinazolin-4-yl)-3,8-diazabicyclo[3.2.1]octane-8-carboxylate (160 mg, 0.2 mmol, 1 eq), tert-butyl (3-cyano-7-fluoro-4-(4,4,5,5-tetramethyl-1,3,2-dioxaborolan-2-yl)benzo[b]thiophen-2-yl)carbamate (138 mg, 0.3 mmol, 1.5 eq), N,N-diisopropylethylamine (85 mg, 0.66 mmol, 3 eq), ditert-butyl(cyclopentyl)phosphane; dichloropalladium; iron (29 mg, 0.04 mmol, 0.2 eq) in water (1 mL) and 2-methyltetrahydrofuran (5 mL) was degassed and purged with nitrogen for 3 times, and then the mixture was stirred at 70° C. for 2 h under N₂ atmosphere. LCMS showed the starting material was consumed completely and one main peak with desired mass was detected. The reaction mixture was diluted with water 10 mL and extracted with ethyl acetate (10 mL×3). The combined organic layers were washed with brine (10 mL×3), dried over anhydrous sodium sulfate, filtered. The filtrate was concentrated under reduced pressure to give a residue. The residue was purified by prep-HPLC (column: Phenomenex luna C18 150*25 mm*10 um; mobile phase: [water (formic acid)-acetonitrile]; B %: 38%-68%, 10 min) to afford the product (72 mg, 0.07 mmol, 33% yield) as a yellow solid. ¹H NMR (400 MHz, DMSO-d₆) (7.95-7.90 (m, 2H), 7.28-7.16 (m, 2H), 7.06 (d, J=4.8 Hz, 2H), 4.53-4.43 (m, 4H), 4.26 (d, J=2.4 Hz, 5H), 3.71 (d, J=11.2 Hz, 2H), 3.54 (d, J=1.6 Hz, 6H), 3.41-3.37 (m, 4H), 2.30 (d, J=2.0 Hz, 1H), 2.29 (d, J=2.4 Hz, 2H), 2.27 (d, J=2.4 Hz, 1H), 1.81 (s, 4H), 1.70 (dd, J=3.6, 6.4 Hz, 4H), 1.47 (s, 9H), 1.44 (s, 9H).

Step 8: 4-(3-((2S)-2-(((4-(8-(tert-butoxycarbonyl)-3,8-diazabicyclo[3.2.1]octan-3-yl)-7-(2-((tert-butoxycarbonyl)amino)-3-cyano-7-fluorobenzo[b]thiophen-4-yl)-6-chloro-8-fluoroquinazolin-2-yl)oxy)methyl)pyrrolidin-1-yl)propoxy)butanoic acid

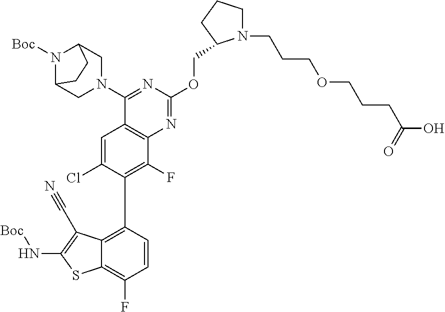 [SMILES: CC(C)(C)OC(=O)Nc1sc2c(F)ccc(-c3c(Cl)cc4c(N5CC6CCC(C5)N6C(=O)OC(C)(C)C)nc(OC[C@@H]5CCCN5CCCOCCCC(=O)O)nc4c3F)c2c1C#N]

To a solution of tert-butyl 3-(7-(2-((tert-butoxycarbonyl)amino)-3-cyano-7-fluorobenzo[b]thiophen-4-yl)-6-chloro-8-fluoro-2-(((S)-1-(3-(4-methoxy-4-oxobutoxy)propyl)pyrrolidin-2-yl)methoxy)quinazolin-4-yl)-3,8-diazabicyclo[3.2.1]octane-8-carboxylate (320 mg, 0.3 mmol, 1 eq) in the mixture solvents of tetrahydrofuran (1 mL), methanol (1 mL) and water (1 mL) was added lithium hydroxide (143 mg, 3 mmol, 10 eq). The mixture was stirred at 20° C. for 2 h. LCMS showed the starting material was consumed completely and one main peak with desired mass was detected. The reaction mixture was concentrated under reduced pressure to remove solvent. The resulting mixture was dissolved in water (10 mL) and adjusted to pH =5 with hydrochloric acid, the mixture was extracted by ethyl acetate (30 mL×3), and the combined organic layers were washed with brine (30 mL×3), dried over anhydrous sodium sulfate. The mixture was filtered and the filtrate was concentrated under reduced pressure to give a residue. The residue was purified by prep-TLC (dichloromethane/methanol=8/1) to afford the product (150 mg, 0.15 mmol, 43% yield) as a yellow solid. LCMS (ESI, m/z): 926.1 [M+H]⁺.

Step 9: tert-butyl 3-(7-(2-((tert-butoxycarbonyl)amino)-3-cyano-7-fluorobenzo[b]thiophen-4-yl)-6-chloro-8-fluoro-2-(((S)-1-(3-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutoxy)propyl)pyrrolidin-2-yl)methoxy)quinazolin-4-yl)-3,8-diazabicyclo[3.2.1]octane-8-carboxylate

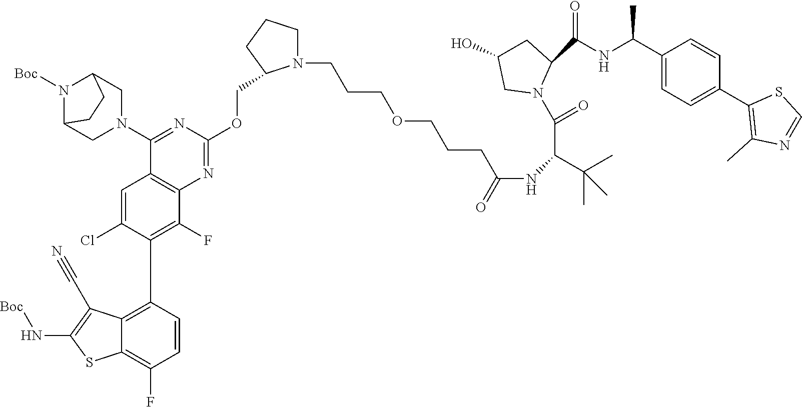 [SMILES: Cc1ncsc1-c1ccc([C@H](C)NC(=O)[C@@H]2C[C@@H](O)CN2C(=O)[C@@H](NC(=O)CCCOCCCN2CCC[C@H]2COc2nc(N3CC4CCC(C3)N4C(=O)OC(C)(C)C)c3cc(Cl)c(-c4ccc(F)c5sc(NC(=O)OC(C)(C)C)c(C#N)c45)c(F)c3n2)C(C)(C)C)cc1]

To a solution of 4-(3-((2S)-2-(((4-(8-(tert-butoxycarbonyl)-3,8-diazabicyclo[3.2.1]octan-3-yl)-7-(2-((tert-butoxycarbonyl)amino)-3-cyano-7-fluorobenzo[b]thiophen-4-yl)-6-chloro-8-fluoroquinazolin-2-yl)oxy)methyl)pyrrolidin-1-yl)propoxy)butanoic acid (68 mg, 73.40 umol, 1 eq) and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (71 mg, 0.15 mmol, 2 eq, hydrochloride) in N,N-dimethylformamide (3 mL) was added N,N-diisopropylethylamine (95 mg, 0.7 mmol, 10 eq), 1-hydroxybenzotriazole (20 mg, 0.15 mmol, 2 eq) and N-(3-dimethylaminopropyl)-N-ethylcarbodiimide hydrochloride (35 mg, 0.18 mmol, 2.5 eq). The mixture was stirred at 20° C. for 12 h. LCMS showed the starting material was consumed completely and one main peak with desired mass was detected. The reaction mixture was diluted with water (10 mL) and extracted with ethyl acetate (10 mL×3). The combined organic layers were washed with brine (10 mL×3), dried over anhydrous sodium sulfate, filtered. The filtrate concentrated under reduced pressure to give a residue. The residue was purified by prep-TLC (dichloromethane/methanol=10/1) to afford the product (40 mg, 0.03 mmol, 36% yield, 90% purity) as a white solid. LCMS (ESI, m/z): 1352.8 [M+H]⁺.

Step 10: (2S,4R)-1-((2S)-2-(4-(3-((2S)-2-(((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)pyrrolidin-1-yl)propoxy)butanamido)-3,3-dimethylbutanoyl)-4-hydroxy-N—((S)-1-(4-(4-methylthiazol-5-yl)phenyl)ethyl)pyrrolidine-2-carboxamide (Example 38)

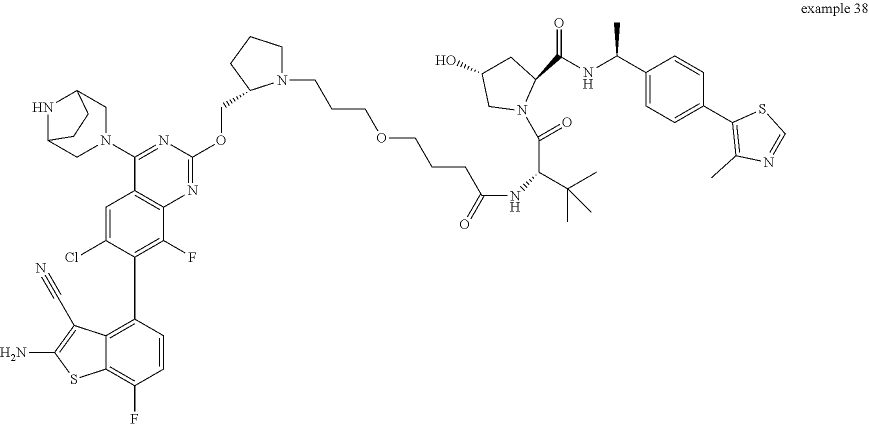 [SMILES: Cc1ncsc1-c1ccc([C@H](C)NC(=O)[C@@H]2C[C@@H](O)CN2C(=O)[C@@H](NC(=O)CCCOCCCN2CCC[C@H]2COc2nc(N3CC4CCC(C3)N4)c3cc(Cl)c(-c4ccc(F)c5sc(N)c(C#N)c45)c(F)c3n2)C(C)(C)C)cc1]

To a solution of tert-butyl 3-(7-(2-((tert-butoxycarbonyl)amino)-3-cyano-7-fluorobenzo[b]thiophen-4-yl)-6-chloro-8-fluoro-2-(((S)-1-(3-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutoxy)propyl)pyrrolidin-2-yl)methoxy)quinazolin-4-yl)-3,8-diazabicyclo[3.2.1]octane-8-carboxylate (40 mg, 0.3 mmol, 1 eq) in dichloromethane (3 mL) was added trifluoroacetic acid (154 mg, 1.35 mmol, 46 eq). The mixture was stirred at 20° C. for 1 h. LCMS showed the starting material was consumed completely and one main peak with desired mass was detected. The reaction mixture was concentrated under reduced pressure to remove the solvent. The residue was purified by prep-HPLC (column: Phenomenex luna C18 150*25 mm*10 um; mobile phase: [water (formic acid)—acetonitrile]; B %: 12%-42%, 2 min) to afford the desired product (15.84 mg, 0.01 mmol, 46% yield) as a white solid. LCMS (ESI, m/z): 1235.1 [M]⁺. ¹H NMR (400 MHz, DMSO-d₆) δ 9.03-8.94 (m, 1H), 8.41-8.32 (m, 1H), 8.19 (s, 1H), 8.13-8.08 (m, 1H), 7.84 (s, 1H), 7.78 (d, J=8.8 Hz, 1H), 7.45-7.41 (m, 2H), 7.40-7.35 (m, 2H), 7.28-7.23 (m, 1H), 7.18-7.11 (m, 1H), 4.94-4.88 (m, 1H), 4.52-4.47 (m, 1H), 4.45-4.40 (m, 1H), 4.38-4.32 (m, 2H), 4.28 (d, J=3.6 Hz, 2H), 4.09-4.03 (m, 2H), 3.62-3.58 (m, 6H), 3.37-3.35 (m, 4H), 3.29-3.27 (m, 2H), 3.07-3.03 (m, 2H), 2.92-2.88 (m, 2H), 2.81 (dd, J=2.4, 6.4 Hz, 2H), 2.45 (d, J=2.0 Hz, 3H), 2.04-1.96 (m, 2H), 1.95-1.86 (m, 2H), 1.81-1.77 (m, 1H), 1.67 (dd, J=5.6, 6.4 Hz, 8H), 1.36 (d, J=6.0 Hz, 3H), 0.91 (s, 9H).

Example 39: 2-amino-(S)-4-[4-(3,8-diazabicyclo[3.2.1]octan-3-yl)-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile

Synthesis of tert-butyl (1R,5S)-3-(7-(2-((tert-butoxycarbonyl)amino)-3-cyano-7-fluorobenzo[b]thiophen-4-yl)-2,8-difluoro-6-(trifluoromethyl)quinazolin-4-yl)-3,8-diazabicyclo[3.2.1]octane-8-carboxylate (Intermediate 1)

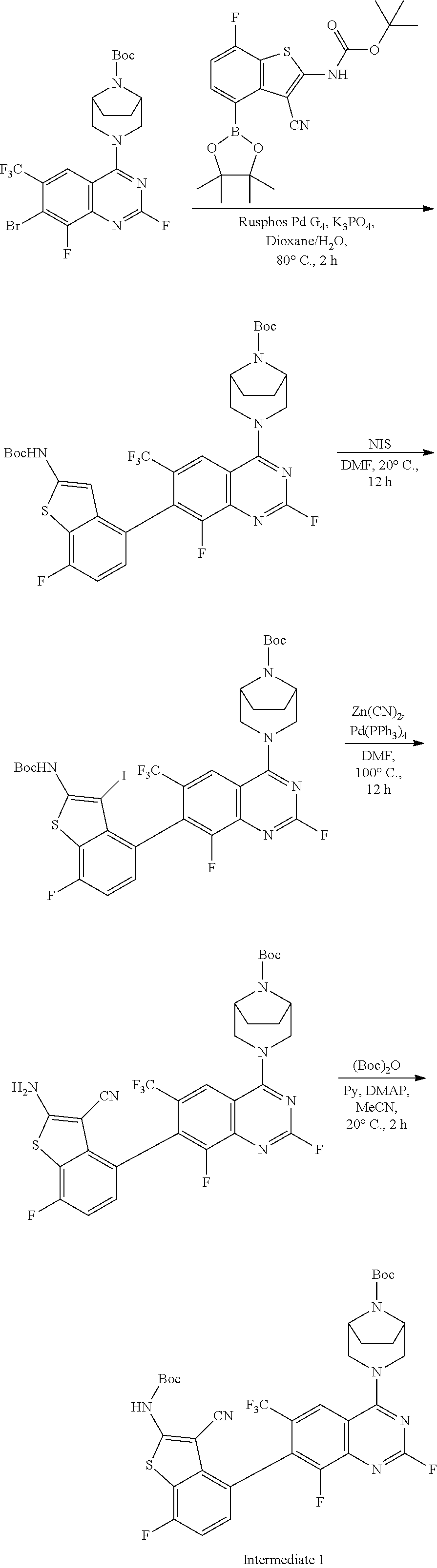

Step 1: tert-butyl (1R,5S)-3-(7-(2-((tert-butoxycarbonyl)amino)-7-fluorobenzo[b]thiophen-4-yl)-2,8-difluoro-6-(trifluoromethyl)quinazolin-4-yl)-3,8-diazabicyclo [3.2.1]octane-8-carboxylate

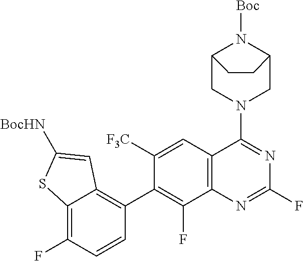 [SMILES: CC(C)(C)OC(=O)Nc1cc2c(-c3c(C(F)(F)F)cc4c(N5CC6CCC(C5)N6C(=O)OC(C)(C)C)nc(F)nc4c3F)ccc(F)c2s1]

A mixture of tert-butyl (1R,5S)-3-(7-bromo-2,8-difluoro-6-(trifluoromethyl)quinazolin-4-yl)-3,8-diazabicyclo[3.2.1]octane-8-carboxylate (4 g, 7.64 mmol, 1 eq), tert-butyl (7-fluoro-4-(4,4,5,5-tetramethyl-1,3,2-dioxaborolan-2-yl)benzo[b]thiophen-2-yl)carbamate (4.51 g, 11.47 mmol, 1.5 eq), potassium phosphate (4.87 g, 22.93 mmol, 3 eq), Rusphos Pd G4 (650 mg, 0.76 mmol, 0.1 eq) in the mixed solvent of dioxane (50 mL) and water (10 mL) was degassed and purged with N₂ for 3 times, and then the mixture was stirred at 80° C. for 2 h under N₂ atmosphere. LCMS showed the starting material was consumed completely and one main peak with desired mass was detected. The reaction mixture was diluted with water (60 mL) and extracted with ethyl acetate (60 mL×3). The combined organic layers were washed with brine (30 mL×3), dried over anhydrous sodium sulfate, filtered and concentrated under reduced pressure to give a residue. The residue was purified by flash silica gel chromatography (ISCO®; 40 g SepaFlash® Silica Flash Column, Eluent of 0˜30% Ethyl acetate/Petroleum ethergradient @ 100 mL/min) to afford the product (5.3 g, 6.87 mmol, 89% yield, 92% purity) as a yellow solid. LCMS (ESI, m/z): 710.0 [M+H]⁺. ¹H NMR: (400 MHz, DMSO-d₆) δ 10.85 (s, 1H), 8.34 (s, 1H), 7.32-7.15 (m, 2H), 6.30 (d, J=3.6 Hz, 1H), 4.64-4.37 (m, 2H), 4.31 (s, 2H), 3.86-3.64 (m, 2H), 1.88-1.57 (m, 4H), 1.47 (d, J=3.2 Hz, 18H)

Step 2: tert-butyl (1R,5S)-3-(7-(2-((tert-butoxycarbonyl)amino)-7-fluoro-3-iodobenzo[b] thiophen-4-yl)-2,8-difluoro-6-(trifluoromethyl)quinazolin-4-yl)-3,8-diazabicyclo[3.2.1]octane-8-carboxylate

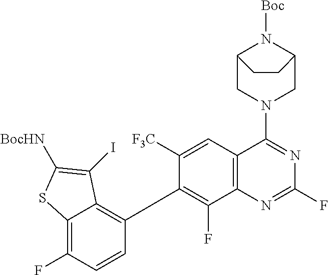 [SMILES: CC(C)(C)OC(=O)Nc1sc2c(F)ccc(-c3c(C(F)(F)F)cc4c(N5CC6CCC(C5)N6C(=O)OC(C)(C)C)nc(F)nc4c3F)c2c1I]

To a solution of tert-butyl (1R,5S)-3-(7-(2-((tert-butoxycarbonyl)amino)-7-fluorobenzo[b] thiophen-4-yl)-2,8-difluoro-6-(trifluoromethyl)quinazolin-4-yl)-3,8-diazabicyclo[3.2.1]octane-8-carboxylate (4.3 g, 6.06 mmol, 1 eq) in N,N-dimethylformamide (50 mL) was added N-Iodosuccinimide (2.04 g, 9.09 mmol, 1.5 eq). The mixture was stirred at 20° C. for 12 h. LCMS showed the starting material was consumed completely and one main peak with desired mass was detected. The reaction mixture was partitioned between ethyl acetate (50 mL) and saturated aqueous sodium thiosulfate (50 mL). The organic phase was separated, washed with saturated aqueous sodium thiosulfate (50 mL×3) and brine (50 ml×3), dried over anhydrous sodium sulfate, filtered and concentrated under reduced pressure to give a residue. The residue was purified by prep-HPLC (column: Phenomenex luna C18 250*50 mm*10 um; mobile phase: [water(FA)-ACN]; B %: 70%-100%, 20 min) to afford the product (3.48 g, 4.12 mmol, 68% yield, 99% purity) as a brown solid. LCMS: MS (ESI) m/z: 835.8 [M+H]⁺. 1H NMR: (400 MHz, CHLOROFORM-d) δ 8.13 (s, 1H), 7.40 (s, 1H), 7.20-7.12 (m, 1H), 7.10-7.00 (m, 1H), 4.75-4.35 (m, 4H), 4.02-3.58 (m, 2H), 2.07-1.95 (m, 2H), 1.87-1.71 (m, 2H), 1.60-1.50 (m, 18H)

Step 3: tert-butyl (1R,5S)-3-(7-(2-amino-3-cyano-7-fluorobenzo[b]thiophen-4-yl)-2,8-difluoro-6-(trifluoromethyl)quinazolin-4-yl)-3,8-diazabicyclo[3.2.1]octane-8-carboxylate

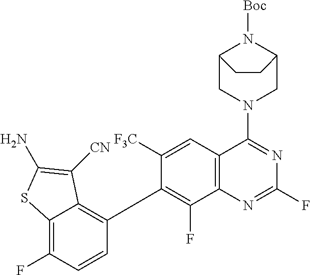 [SMILES: CC(C)(C)OC(=O)N1C2CCC1CN(c1nc(F)nc3c(F)c(-c4ccc(F)c5sc(N)c(C#N)c45)c(C(F)(F)F)cc13)C2]

A mixture of tert-butyl (1R,5S)-3-(7-(2-((tert-butoxycarbonyl)amino)-7-fluoro-3-iodobenzo[b] thiophen-4-yl)-2,8-difluoro-6-(trifluoromethyl)quinazolin-4-yl)-3,8-diazabicyclo[3.2.1]octane-8-carboxylate (350 mg, 0.42 mmol, 1 eq), zinc cyanide (180 mg, 1.53 mmol, 3.66 eq), tetrakis[triphenylphosphine]palladium(0) (96 mg, 0.083 mmol, 0.2 eq) in N,N-dimethylformamide (10 mL) was degassed and purged with N₂ for 3 times, and then the mixture was stirred at 100° C. for 12 h under N₂ atmosphere. LCMS showed the starting material was consumed completely and one main peak with desired mass was detected. The mixture were diluted with saturated sodium bicarbonate solution (40 mL) and extracted with ethyl acetate (40 mL). The organic layer was washed with water (40 mL), brine (40 mL), dried over anhydrous sodium sulfate and then concentrated to give a residue. The residue was purified by prep-TLC (Petroleum ether/Ethyl acetate=3/1) to afford the desired product (236 mg, 0.29 mmol, 71% yield, 80% purity) as a white solid. LCMS (ESI, m/z): 635.0 [M+H]⁺. ¹H NMR: (400 MHz, CHLOROFORM-d) (8.09 (s, 1H), 7.24 (dd, J=4.8, 8.4 Hz, 1H), 7.04 (t, J=8.8 Hz, 1H), 5.33 (s, 2H), 4.65-4.36 (m, 4H), 3.96-3.58 (m, 2H), 1.97 (s, 2H), 1.83-1.68 (m, 2H), 1.54 (s, 9H)

Step 4: tert-butyl (1R,5S)-3-(7-(2-((tert-butoxycarbonyl)amino)-3-cyano-7-fluorobenzo [b]thiophen-4-yl)-2,8-difluoro-6-(trifluoromethyl)quinazolin-4-yl)-3,8-diazabicyclo[3.2.1]octane-8-carboxylate

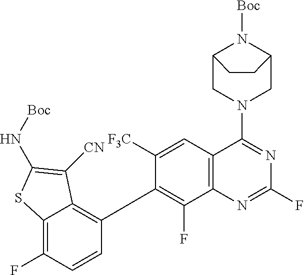 [SMILES: CC(C)(C)OC(=O)Nc1sc2c(F)ccc(-c3c(C(F)(F)F)cc4c(N5CC6CCC(C5)N6C(=O)OC(C)(C)C)nc(F)nc4c3F)c2c1C#N]

To a solution of tert-butyl (1R,5S)-3-(7-(2-amino-3-cyano-7-fluorobenzo[b]thiophen-4-yl)-2,8-difluoro-6-(trifluoromethyl)quinazolin-4-yl)-3,8-diazabicyclo[3.2.1]octane-8-carboxylate (100 mg, 0.15 mmol, 1 eq) in acetonitrile (3 mL) was added dimethylaminopyridine 4-dimethylaminopyridine (2 mg, 0.015 mmol, 0.1 eq), pyridine (147 mg, 1.86 mmol, 0.15 mL, 11.79 eq) and di-tert-butyl dicarbonate (51 mg, 0.23 mmol, 1.5 eq). The mixture was stirred at 20° C. for 2 h. LCMS showed one main peak with desired mass was detected. The reaction mixture was diluted with water (15 mL) and extracted with ethyl acetate (15 mL×3). The combined organic layers were washed with aqueous hydrochloride (1 M, 15 mL) and brine (15 mL×2), dried over anhydrous sodium sulfate, filtered and concentrated under reduced pressure to give a residue. The residue was purified by prep-TLC (petroleum ether/ethyl acetate=3/1) to afford the desired product (88 mg, 012 mmol, 76% yield) as a white solid. LCMS: MS (ESI) m z: 734.9 [M+H]⁺. ¹H NMR (400 MHz, CHLOROFORM-d) δ 8.10 (s, 1H), 7.79 (s, 1H), 7.32 (dd, J=4.4, 8.4 Hz, 1H), 7.15 (t, J=8.8 Hz, 1H), 4.72-4.32 (m, 4H), 3.94-3.58 (m, 2H), 2.03-1.90 (m, 2H), 1.86-1.69 (m, 2H), 1.56 (s, 9H), 1.54 (s, 9H)

Synthesis of 2-amino-(S)-4-[4-(3,8-diazabicyclo[3.2.1]octan-3-yl)-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (Example 39)

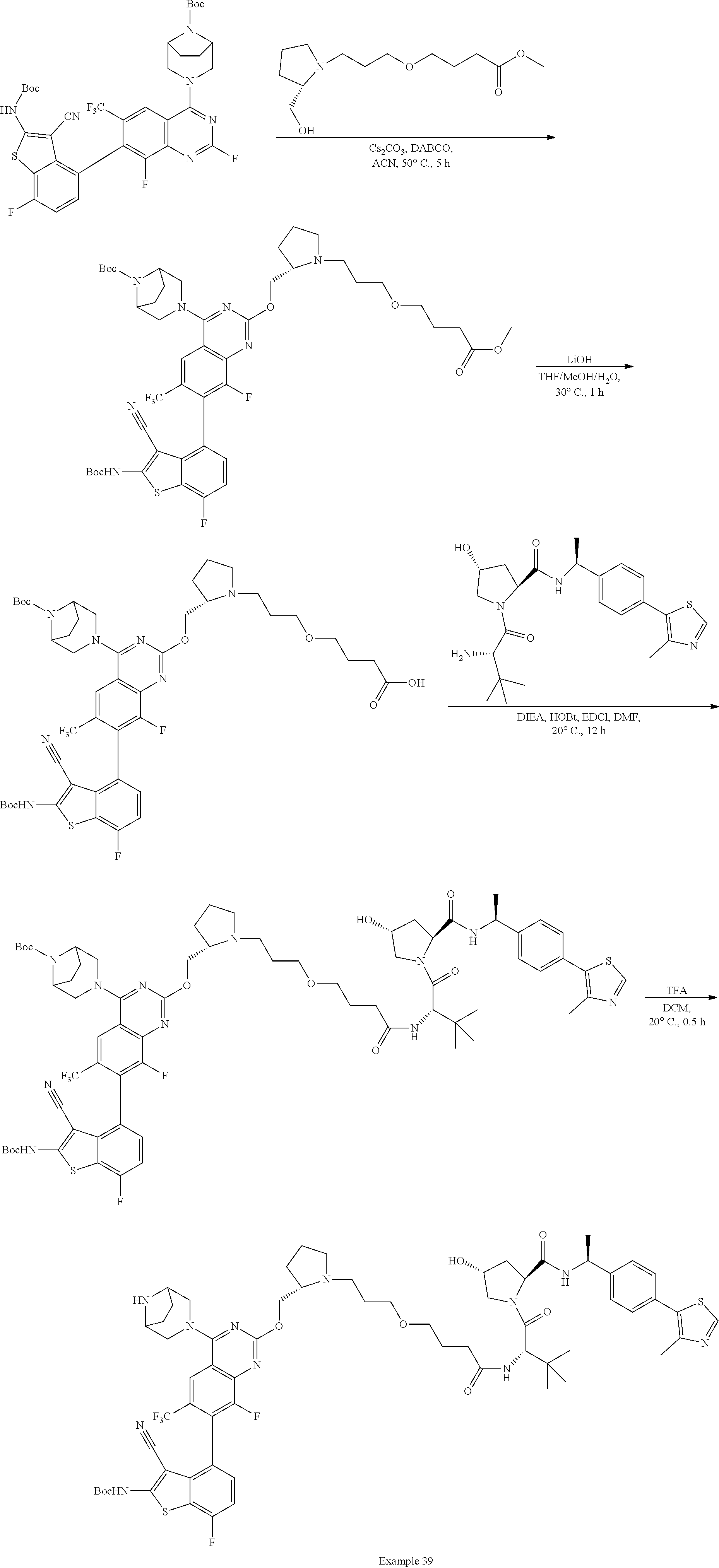

Step 1: tert-butyl 3-(7-(2-((tert-butoxycarbonyl)amino)-3-cyano-7-fluorobenzo[b]thiophen-4-yl)-8-fluoro-2-(((S)-1-(3-(4-methoxy-4-oxobutoxy)propyl)pyrrolidin-2-yl)methoxy)-6-(trifluoromethyl)quinazolin-4-yl)-3,8-diazabicyclo [3.2.1]octane-8-carboxylate

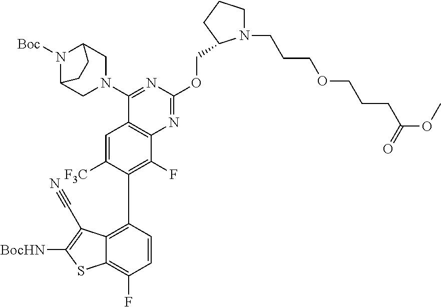 [SMILES: COC(=O)CCCOCCCN1CCC[C@H]1COc1nc(N2CC3CCC(C2)N3C(=O)OC(C)(C)C)c2cc(C(F)(F)F)c(-c3ccc(F)c4sc(NC(=O)OC(C)(C)C)c(C#N)c34)c(F)c2n1]

To a solution of tert-butyl (1R,5S)-3-(7-(2-((tert-butoxycarbonyl)amino)-3-cyano-7-fluorobenzo[b]thiophen-4-yl)-2,8-difluoro-6-(trifluoromethyl)quinazolin-4-yl)-3,8-diazabicyclo[3.2.1]octane-8-carboxylate(100 mg, 0.13 mmol, 1 eq) and methyl (S)-4-(3-(2-(hydroxymethyl)pyrrolidin-1-yl)propoxy)butanoate (70 mg, 0.27 mmol, 2 eq) in acetonitrile (2 mL) was added cesium carbonate (88 mg, 0.27 mmol, 2 eq) and 1,4-diazabicyclo[2.2.2]octane (1 mg, 0.013 mmol, 0.1 eq). The mixture was stirred at 50° C. for 5 h. LCMS showed the starting material was consumed completely and one main peak with desired mass was detected. The reaction mixture was diluted with water (15 mL) and extracted with ethyl acetate (15 mL×3). The combined organic layers were washed with brine (15 mL×2), dried over anhydrous sodium sulfate, filtered and concentrated under reduced pressure to give a residue. The residue was purified by prep-TLC (dichloromethane/methanol=20/1) to afford the desired product (65 mg, 0.066 mmol, 49% yield) as a white solid. LCMS (ESI, m/z): 974.0 [M+H]⁺.

Step 2: 4-(3-((2S)-2-(((4-(8-(tert-butoxycarbonyl)-3,8-diazabicyclo[3.2.1]octan-3-yl)-7-(2-((tert-butoxycarbonyl)amino)-3-cyano-7-fluorobenzo[b]thiophen-4-yl)-8-fluoro-6-(trifluoromethyl)quinazolin-2-yl)oxy)methyl)pyrrolidin-1-yl)propoxy)butanoic acid

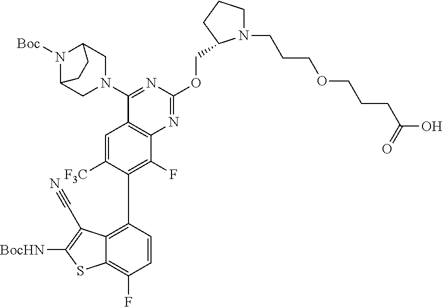 [SMILES: CC(C)(C)OC(=O)Nc1sc2c(F)ccc(-c3c(C(F)(F)F)cc4c(N5CC6CCC(C5)N6C(=O)OC(C)(C)C)nc(OC[C@@H]5CCCN5CCCOCCCC(=O)O)nc4c3F)c2c1C#N]

To a solution of tert-butyl 3-(7-(2-((tert-butoxycarbonyl)amino)-3-cyano-7-fluorobenzo[b]thiophen-4-yl)-8-fluoro-2-(((S)-1-(3-(4-methoxy-4-oxobutoxy)propyl)pyrrolidin-2-yl)methoxy)-6-(trifluoromethyl)quinazolin-4-yl)-3,8-diazabicyclo[3.2.1]octane-8-carboxylate (65 mg, 0.066 mmol, 1 eq) in methanol (1 mL) was added lithium hydroxide (14 mg, 0.33 mmol, 5 eq), tetrahydrofuran (1 mL) and water (1 mL). The mixture was stirred at 30° C. for 1 h. LCMS showed the starting material was consumed completely and one main peak with desired mass was detected. The reaction mixture was concentrated under reduced pressure to remove solvent. The residue was diluted with water (5 mL) and adjusted pH=4 with hydrochloric acid (1 M), then extracted with ethyl acetate (10 mL×3). The combined organic layers were washed with brine (10 mL×2), dried over anhydrous sodium sulfate, filtered and concentrated under reduced pressure to afford the desired product (64 mg, 0.066 mmol, 99% yield) as a colorless oil. LCMS (ESI, m/z): 960.7 [M+H]⁺.

Step 3: tert-butyl 3-(7-(2-((tert-butoxycarbonyl)amino)-3-cyano-7-fluorobenzo[b]thiophen-4-yl)-8-fluoro-2-(((S)-1-(3-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutoxy)propyl)pyrrolidin-2-yl)methoxy)-6-(trifluoromethyl)quinazolin-4-yl)-3,8-diazabicyclo[3.2.1]octane-8-carboxylate

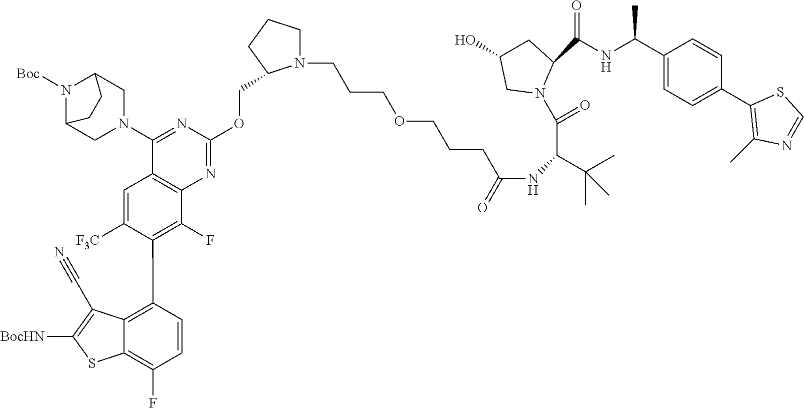 [SMILES: Cc1ncsc1-c1ccc([C@H](C)NC(=O)[C@@H]2C[C@@H](O)CN2C(=O)[C@@H](NC(=O)CCCOCCCN2CCC[C@H]2COc2nc(N3CC4CCC(C3)N4C(=O)OC(C)(C)C)c3cc(C(F)(F)F)c(-c4ccc(F)c5sc(NC(=O)OC(C)(C)C)c(C#N)c45)c(F)c3n2)C(C)(C)C)cc1]

To a solution of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (35 mg, 0.080 mmol, 1.2 eq) in N,N-dimethylformamide (2 mL) was added N,N-diisopropylethylamine (25 mg, 0.20 mmol, 3 eq) and 4-(3-((2S)-2-(((4-(8-(tert-butoxycarbonyl)-3,8-diazabicyclo[3.2.1]octan-3-yl)-7-(2-((tert-butoxycarbonyl)amino)-3-cyano-7-fluorobenzo[b]thiophen-4-yl)-8-fluoro-6-(trifluoromethyl)quinazolin-2-yl)oxy)methyl)pyrrolidin-1-yl)propoxy)butanoic acid (64 mg, 0.066 mmol, 1 eq), 1-hydroxybenzotriazole (18 mg, 0.13 mmol, 2 eq). The mixture was stirred at 20° C. for 30 mn. Then N-(3-dimethylaminopropyl)-N-ethylcarbodiimide hydrochloride (25 mg, 0.13 mmol, 2 eq) was added, the mixture was stirred at 20° C. for 12 h. LCMS showed the starting material was consumed completely and one main peak with desired mass was detected. The reaction mixture was partitioned between ethyl acetate (15 mL) and water (15 mL). The organic phase was separated, washed with brine (15 mL×3), dried over anhydrous sodium sulfate, filtered and concentrated under reduced pressure to give a residue. The residue was purified by prep-TLC (dichloromethane/method=10/1) to afford desired product (45 mg, 0.032 mmol, 48% yield) as a colorless oil. LCMS (ESI, m/z): 1387.1 [M+H]⁺.

Step 4: (2S,4R)-1-((2S)-2-(4-(3-((2S)-2-(((7-(2-amino-3-cyano-7-fluorobenzo [b]thiophen-4-yl)-4-(3,8-diazabicyclo[3.2.1]octan-3-yl)-8-fluoro-6-(trifluoromethyl)quinazolin-2-yl)oxy)methyl)pyrrolidin-1-yl)propoxy)butanamido)-3,3-dimethylbutanoyl)-4-hydroxy-N—((S)-1-(4-(4-methylthiazol-5-yl)phenyl)ethyl)pyrrolidine-2-carboxamide (Example 39)

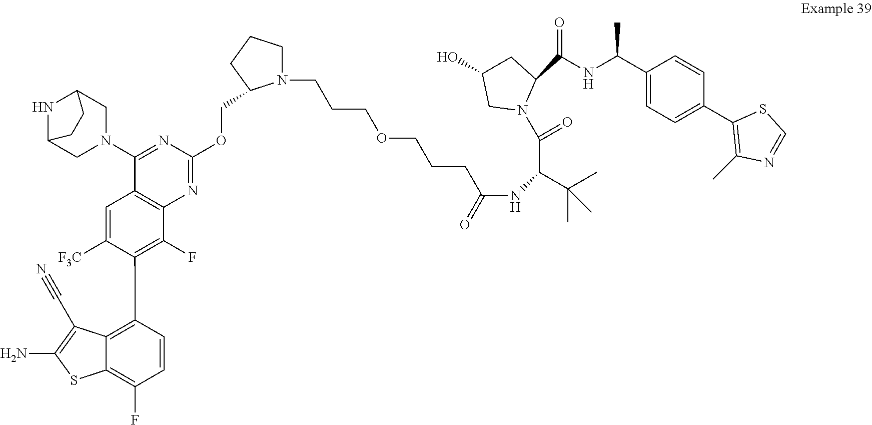 [SMILES: Cc1ncsc1-c1ccc([C@H](C)NC(=O)[C@@H]2C[C@@H](O)CN2C(=O)[C@@H](NC(=O)CCCOCCCN2CCC[C@H]2COc2nc(N3CC4CCC(C3)N4)c3cc(C(F)(F)F)c(-c4ccc(F)c5sc(N)c(C#N)c45)c(F)c3n2)C(C)(C)C)cc1]

To a solution of tert-butyl 3-(7-(2-((tert-butoxycarbonyl)amino)-3-cyano-7-fluorobenzo[b]thiophen-4-yl)-8-fluoro-2-(((S)-1-(3-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutoxy)propyl)pyrrolidin-2-yl)methoxy)-6-(trifluoromethyl)quinazolin-4-yl)-3,8-diazabicyclo[3.2.1]octane-8-carboxylate (45 mg, 0.032 mmol, 1 eq) in dichloromethane (3 mL) was added trifluoroacetic acid (1 mL). The mixture was stirred at 20° C. for 0.5 h. LCMS showed the starting material was consumed completely and one main peak with desired mass was detected. The reaction mixture was concentrated under reduced pressure to give a residue. The residue was purified by prep-HPLC (column: Phenomenex luna C18 150*25 mm*10 um; mobile phase: [water(FA)-ACN]; B %: 14%-44%, 2 min) to afford the desired product (11.22 mg, 0.009 mmol, 27% yield, 99% purity, formate[1]) as a yellow solid. LCMS (ESI, m/z): 1187.4 [M+H]⁺. ¹H NMR: (400 MHz, DMSO-d6) δ 9.01-8.96 (m, 1H), 8.39-8.33 (m, 1H), 8.20 (s, 1H), 8.07 (s, 2H), 7.82-7.75 (m, 1H), 7.49-7.36 (m, 4H), 7.30-7.21 (m, 1H), 7.13 (t, J=8.8 Hz, 1H), 4.96-4.88 (m, 1H), 4.51-4.25 (m, 6H), 4.13-4.04 (m, 1H), 3.63-3.56 (m, 6H), 3.37-3.32 (m, 4H), 3.29-3.26 (m, 2H), 3.09-3.01 (m, 2H), 2.94-2.79 (m, 2H), 2.45 (s, 3H), 2.29-2.09 (m, 4H), 2.04-1.97 (in, 1H), 1.90 (dd, J=7.2, 10.8 Hz, 1H), 1.79 (in, J=4.0, 8.0 Hz, 1H), 1.73-1.58 (in, 10H), 1.37 (d, J=6.8 Hz, 3H), 0.91 (s, 9H).

### Characterization of Further Examples Encompassed by the Application

Examples 40-90

| Ex | |
|---|---|
| # | Structure |
| 40 | 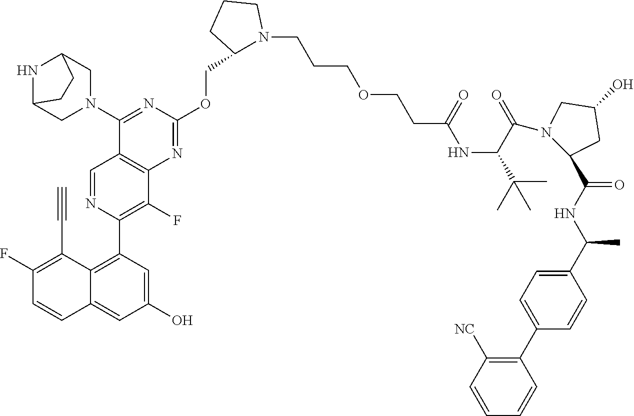 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6ccccc6C#N)cc5)C(C)(C)C)nc4c3F)c12] |
| 41 | 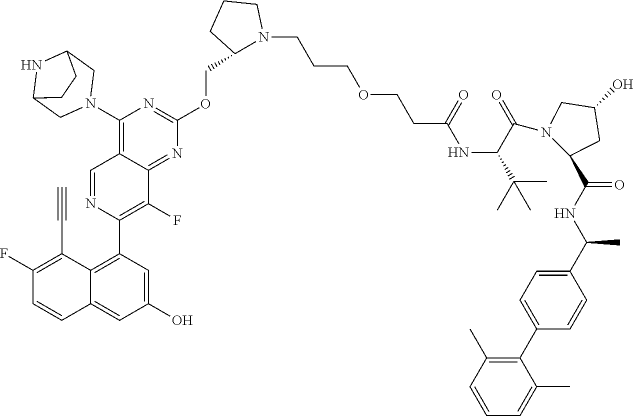 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(C)cccc6C)cc5)C(C)(C)C)nc4c3F)c12] |
| 42 | 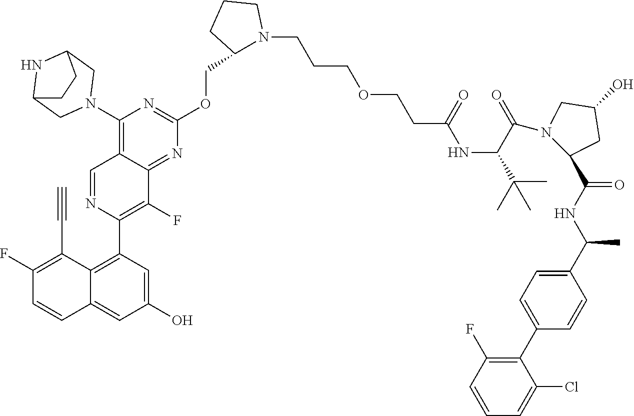 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(F)cccc6Cl)cc5)C(C)(C)C)nc4c3F)c12] |
| 43 | 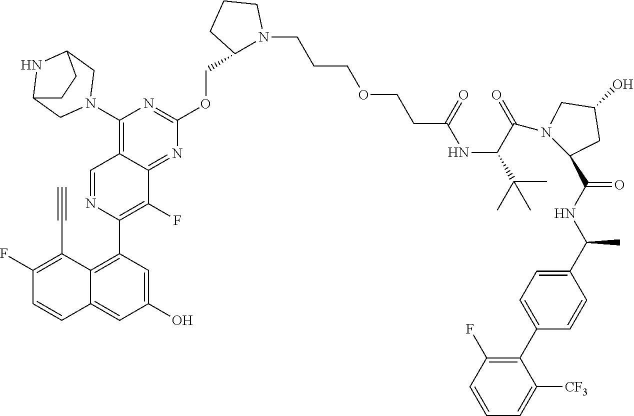 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(F)cccc6C(F)(F)F)cc5)C(C)(C)C)nc4c3F)c12] |
| 44 | 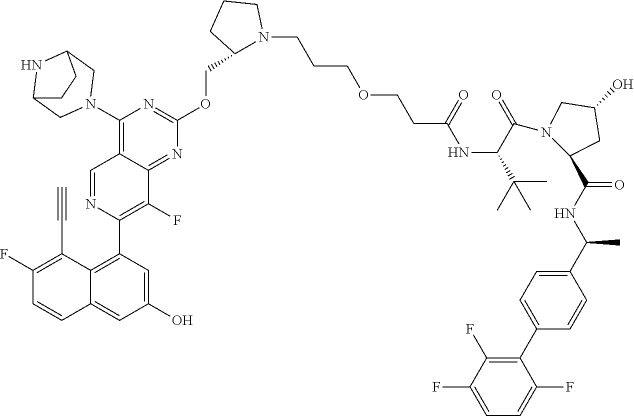 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(F)ccc(F)c6F)cc5)C(C)(C)C)nc4c3F)c12] |
| 45 | 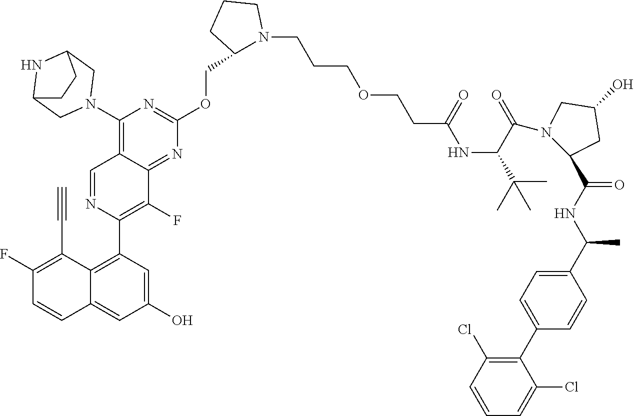 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(Cl)cccc6Cl)cc5)C(C)(C)C)nc4c3F)c12] |
| 46 | 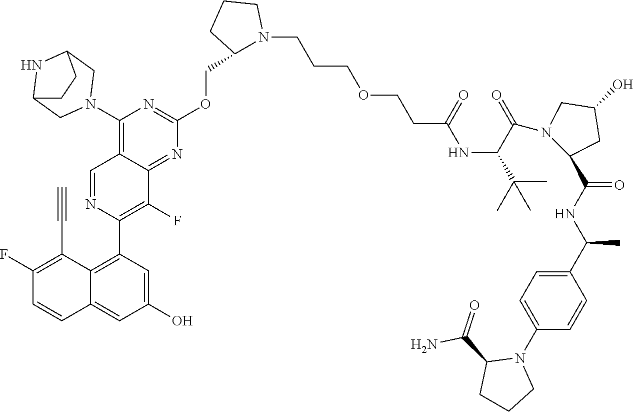 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(N6CCC[C@H]6C(N)=O)cc5)C(C)(C)C)nc4c3F)c12] |
| 47 | 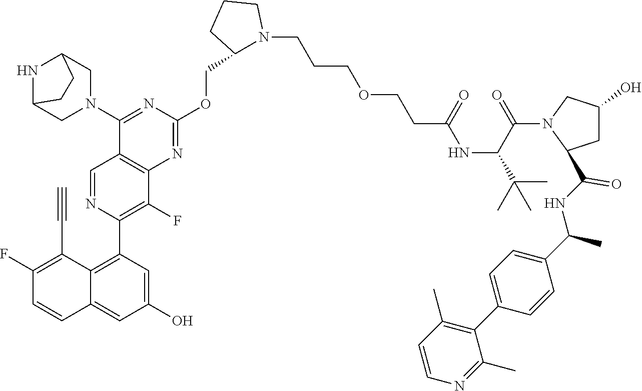 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(C)ccnc6C)cc5)C(C)(C)C)nc4c3F)c12] |
| 48 | 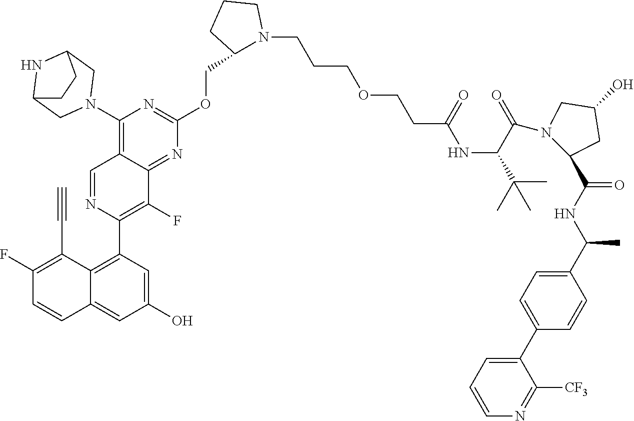 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6cccnc6C(F)(F)F)cc5)C(C)(C)C)nc4c3F)c12] |
| 49 | 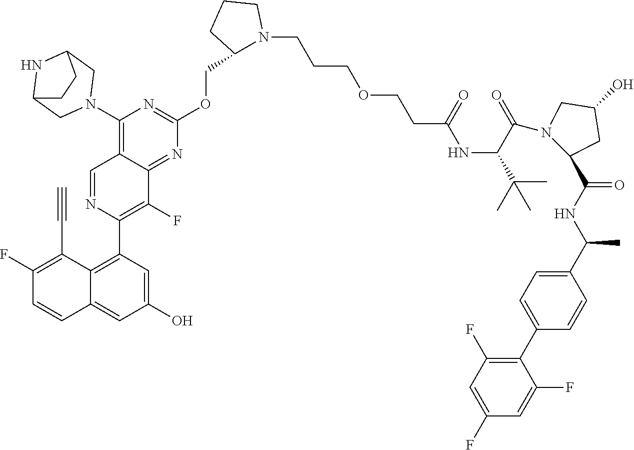 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(F)cc(F)cc6F)cc5)C(C)(C)C)nc4c3F)c12] |
| 50 | 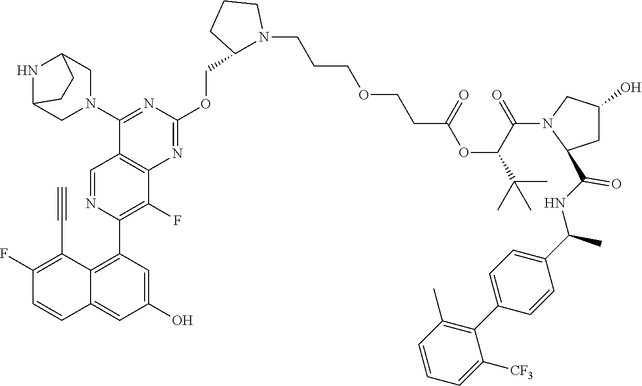 [SMILES: C#Cc1c(F)ccc2cc(O)cc(-c3ncc4c(N5CC6CCC(C5)N6)nc(OC[C@@H]5CCCN5CCCOCCC(=O)O[C@H](C(=O)N5C[C@H](O)C[C@H]5C(=O)N[C@@H](C)c5ccc(-c6c(C)cccc6C(F)(F)F)cc5)C(C)(C)C)nc4c3F)c12] |
| 51 | 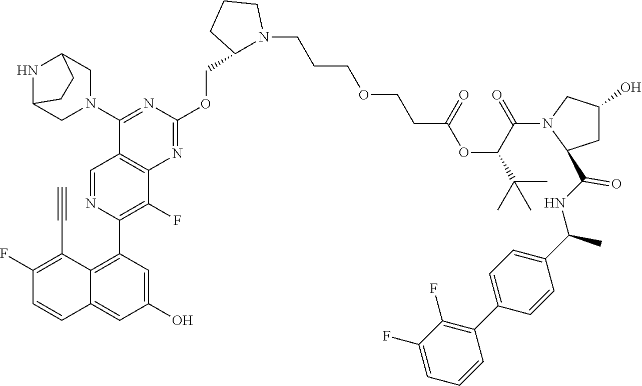 [SMILES: C#Cc1c(F)ccc2cc(O)cc(-c3ncc4c(N5CC6CCC(C5)N6)nc(OC[C@@H]5CCCN5CCCOCCC(=O)O[C@H](C(=O)N5C[C@H](O)C[C@H]5C(=O)N[C@@H](C)c5ccc(-c6cccc(F)c6F)cc5)C(C)(C)C)nc4c3F)c12] |
| 52 | 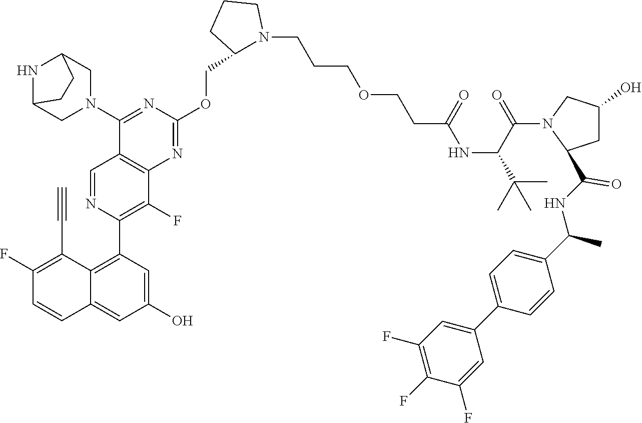 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6cc(F)c(F)c(F)c6)cc5)C(C)(C)C)nc4c3F)c12] |
| 53 | 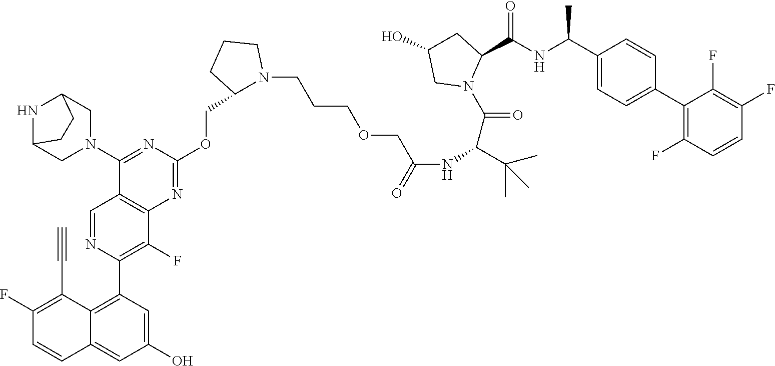 [SMILES: C#Cc1c(F)ccc2cc(O)cc(-c3ncc4c(N5CC6CCC(C5)N6)nc(OC[C@@H]5CCCN5CCCOCC(=O)N[C@H](C(=O)N5C[C@H](O)C[C@H]5C(=O)N[C@@H](C)c5ccc(-c6c(F)ccc(F)c6F)cc5)C(C)(C)C)nc4c3F)c12] |
| 54 | 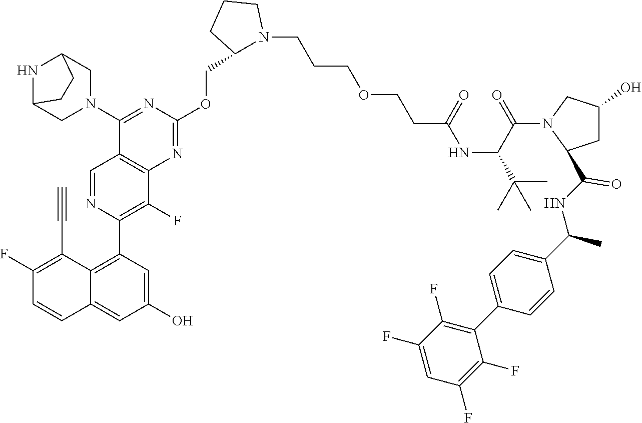 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(F)c(F)cc(F)c6F)cc5)C(C)(C)C)nc4c3F)c12] |
| 55 | 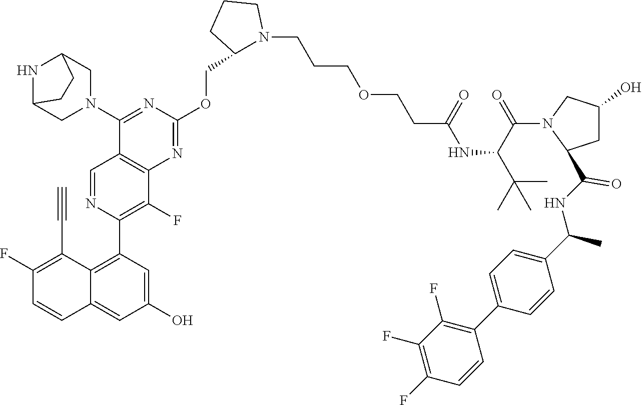 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6ccc(F)c(F)c6F)cc5)C(C)(C)C)nc4c3F)c12] |
| 56 | 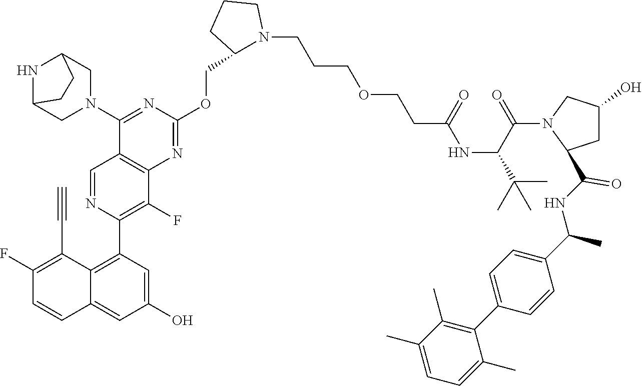 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(C)ccc(C)c6C)cc5)C(C)(C)C)nc4c3F)c12] |
| 57 | 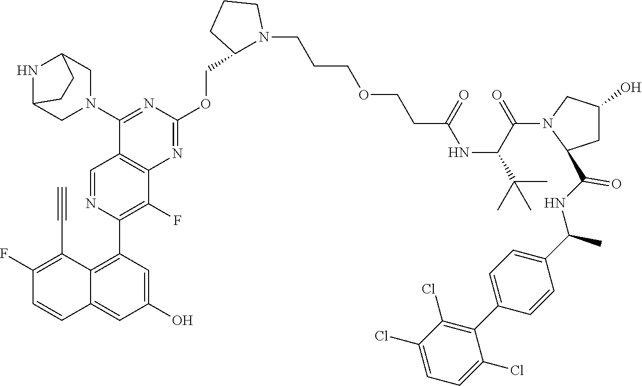 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(Cl)ccc(Cl)c6Cl)cc5)C(C)(C)C)nc4c3F)c12] |
| 58 | 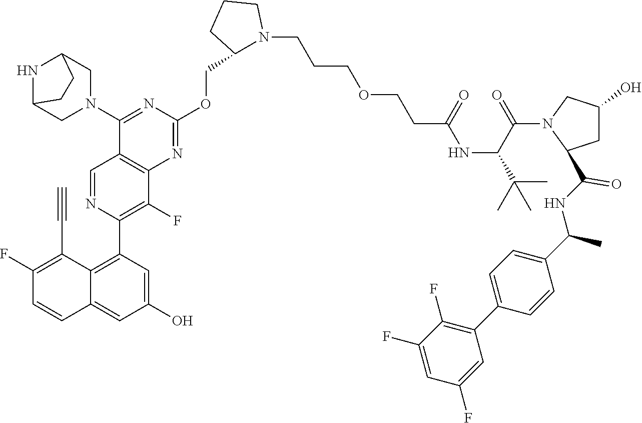 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6cc(F)cc(F)c6F)cc5)C(C)(C)C)nc4c3F)c12] |
| 59 | 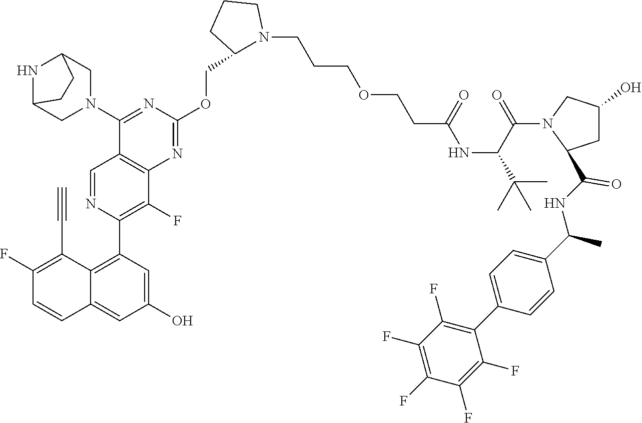 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(F)c(F)c(F)c(F)c6F)cc5)C(C)(C)C)nc4c3F)c12] |
| 60 | 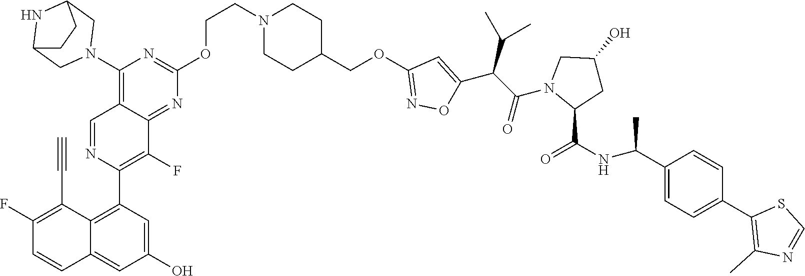 [SMILES: C#Cc1c(F)ccc2cc(O)cc(-c3ncc4c(N5CC6CCC(C5)N6)nc(OCCN5CCC(COc6cc([C@H](C(=O)N7C[C@H](O)C[C@H]7C(=O)N[C@@H](C)c7ccc(-c8scnc8C)cc7)C(C)C)on6)CC5)nc4c3F)c12] |
| 61 | 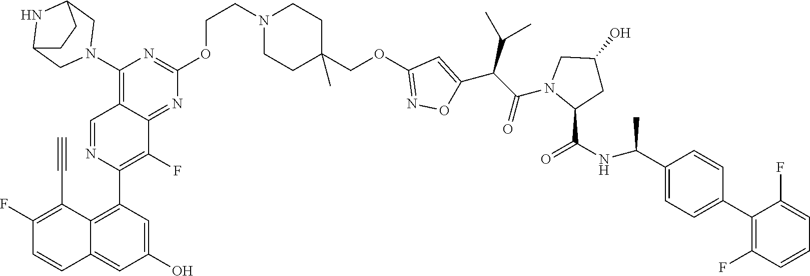 [SMILES: C#Cc1c(F)ccc2cc(O)cc(-c3ncc4c(N5CC6CCC(C5)N6)nc(OCCN5CCC(C)(COc6cc([C@H](C(=O)N7C[C@H](O)C[C@H]7C(=O)N[C@@H](C)c7ccc(-c8c(F)cccc8F)cc7)C(C)C)on6)CC5)nc4c3F)c12] |
| 62 | 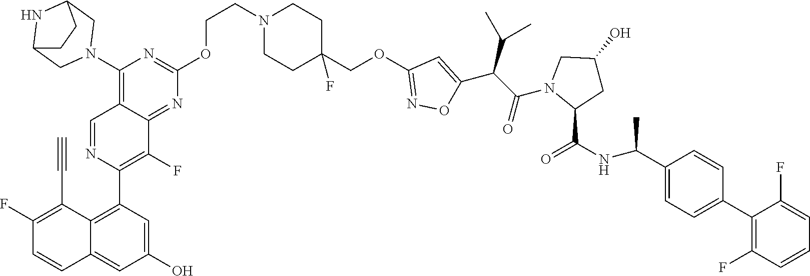 [SMILES: C#Cc1c(F)ccc2cc(O)cc(-c3ncc4c(N5CC6CCC(C5)N6)nc(OCCN5CCC(F)(COc6cc([C@H](C(=O)N7C[C@H](O)C[C@H]7C(=O)N[C@@H](C)c7ccc(-c8c(F)cccc8F)cc7)C(C)C)on6)CC5)nc4c3F)c12] |
| 63 | 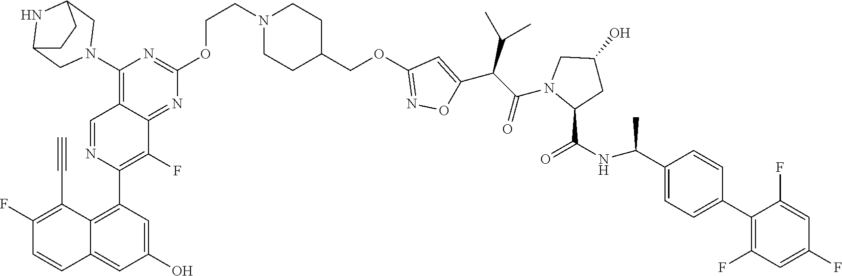 [SMILES: C#Cc1c(F)ccc2cc(O)cc(-c3ncc4c(N5CC6CCC(C5)N6)nc(OCCN5CCC(COc6cc([C@H](C(=O)N7C[C@H](O)C[C@H]7C(=O)N[C@@H](C)c7ccc(-c8c(F)cc(F)cc8F)cc7)C(C)C)on6)CC5)nc4c3F)c12] |
| 64 | 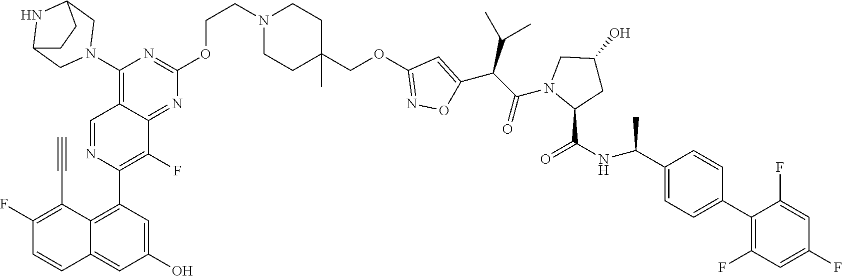 [SMILES: C#Cc1c(F)ccc2cc(O)cc(-c3ncc4c(N5CC6CCC(C5)N6)nc(OCCN5CCC(C)(COc6cc([C@H](C(=O)N7C[C@H](O)C[C@H]7C(=O)N[C@@H](C)c7ccc(-c8c(F)cc(F)cc8F)cc7)C(C)C)on6)CC5)nc4c3F)c12] |
| 65 | 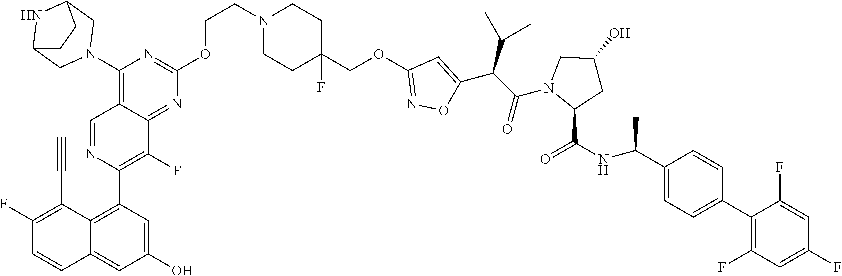 [SMILES: C#Cc1c(F)ccc2cc(O)cc(-c3ncc4c(N5CC6CCC(C5)N6)nc(OCCN5CCC(F)(COc6cc([C@H](C(=O)N7C[C@H](O)C[C@H]7C(=O)N[C@@H](C)c7ccc(-c8c(F)cc(F)cc8F)cc7)C(C)C)on6)CC5)nc4c3F)c12] |
| 66 | 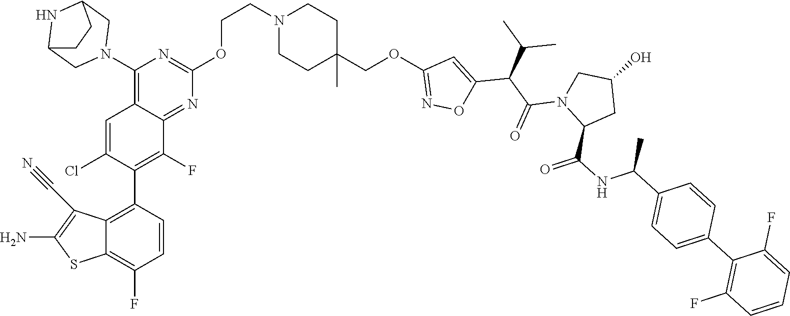 [SMILES: CC(C)[C@@H](C(=O)N1C[C@H](O)C[C@H]1C(=O)N[C@@H](C)c1ccc(-c2c(F)cccc2F)cc1)c1cc(OCC2(C)CCN(CCOc3nc(N4CC5CCC(C4)N5)c4cc(Cl)c(-c5ccc(F)c6sc(N)c(C#N)c56)c(F)c4n3)CC2)no1] |
| 67 | 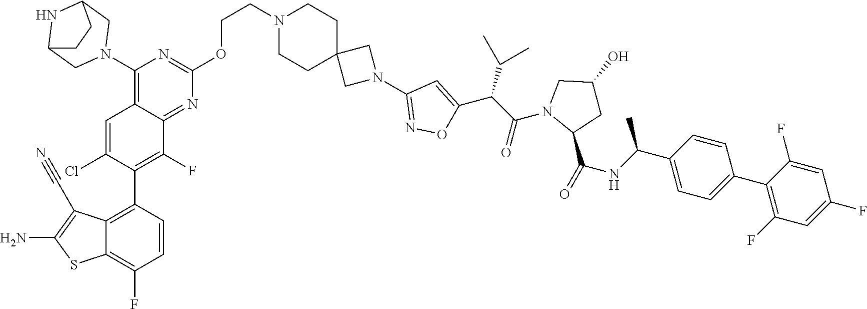 [SMILES: CC(C)[C@H](C(=O)N1C[C@H](O)C[C@H]1C(=O)N[C@@H](C)c1ccc(-c2c(F)cc(F)cc2F)cc1)c1cc(N2CC3(CCN(CCOc4nc(N5CC6CCC(C5)N6)c5cc(Cl)c(-c6ccc(F)c7sc(N)c(C#N)c67)c(F)c5n4)CC3)C2)no1] |
| 68 | 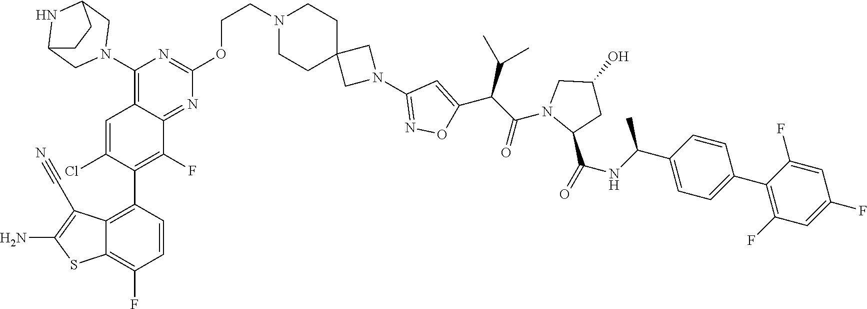 [SMILES: CC(C)[C@@H](C(=O)N1C[C@H](O)C[C@H]1C(=O)N[C@@H](C)c1ccc(-c2c(F)cc(F)cc2F)cc1)c1cc(N2CC3(CCN(CCOc4nc(N5CC6CCC(C5)N6)c5cc(Cl)c(-c6ccc(F)c7sc(N)c(C#N)c67)c(F)c5n4)CC3)C2)no1] |
| 69 | 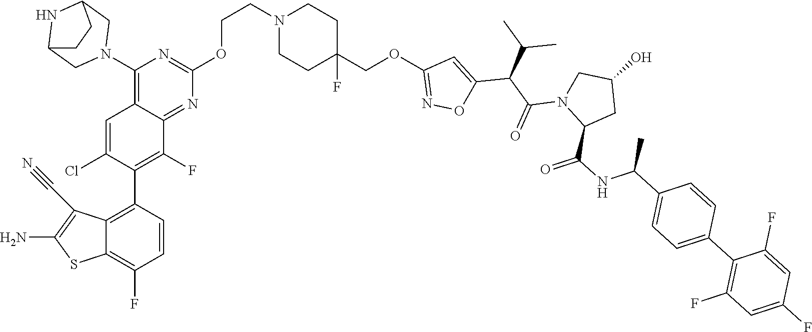 [SMILES: CC(C)[C@@H](C(=O)N1C[C@H](O)C[C@H]1C(=O)N[C@@H](C)c1ccc(-c2c(F)cc(F)cc2F)cc1)c1cc(OCC2(F)CCN(CCOc3nc(N4CC5CCC(C4)N5)c4cc(Cl)c(-c5ccc(F)c6sc(N)c(C#N)c56)c(F)c4n3)CC2)no1] |
| 70 | 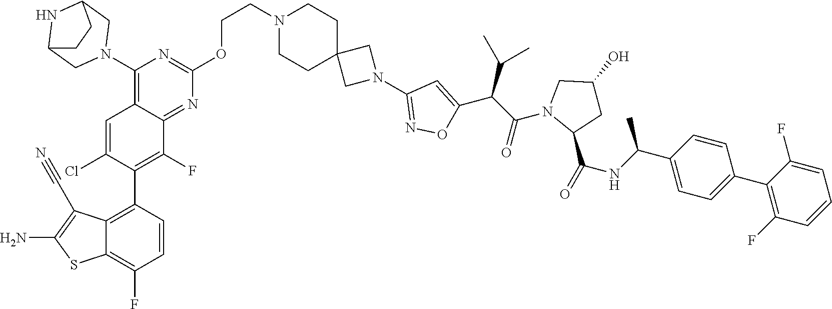 [SMILES: CC(C)[C@@H](C(=O)N1C[C@H](O)C[C@H]1C(=O)N[C@@H](C)c1ccc(-c2c(F)cccc2F)cc1)c1cc(N2CC3(CCN(CCOc4nc(N5CC6CCC(C5)N6)c5cc(Cl)c(-c6ccc(F)c7sc(N)c(C#N)c67)c(F)c5n4)CC3)C2)no1] |
| 71 | 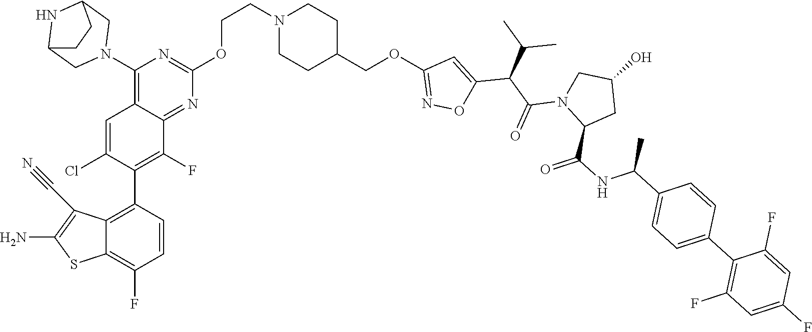 [SMILES: CC(C)[C@@H](C(=O)N1C[C@H](O)C[C@H]1C(=O)N[C@@H](C)c1ccc(-c2c(F)cc(F)cc2F)cc1)c1cc(OCC2CCN(CCOc3nc(N4CC5CCC(C4)N5)c4cc(Cl)c(-c5ccc(F)c6sc(N)c(C#N)c56)c(F)c4n3)CC2)no1] |
| 72 | 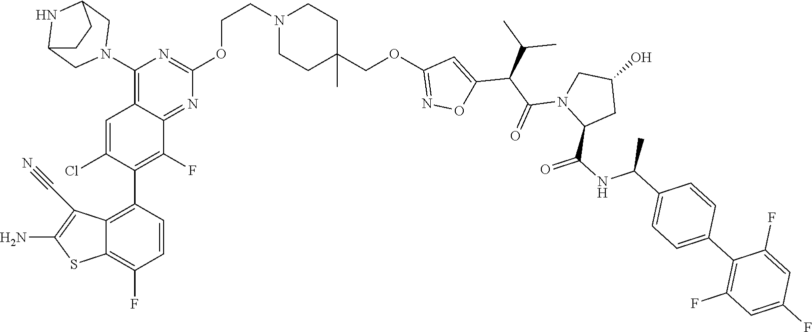 [SMILES: CC(C)[C@@H](C(=O)N1C[C@H](O)C[C@H]1C(=O)N[C@@H](C)c1ccc(-c2c(F)cc(F)cc2F)cc1)c1cc(OCC2(C)CCN(CCOc3nc(N4CC5CCC(C4)N5)c4cc(Cl)c(-c5ccc(F)c6sc(N)c(C#N)c56)c(F)c4n3)CC2)no1] |
| 73 | 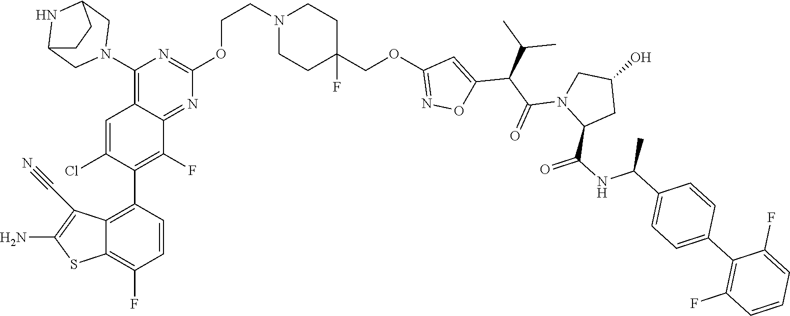 [SMILES: CC(C)[C@@H](C(=O)N1C[C@H](O)C[C@H]1C(=O)N[C@@H](C)c1ccc(-c2c(F)cccc2F)cc1)c1cc(OCC2(F)CCN(CCOc3nc(N4CC5CCC(C4)N5)c4cc(Cl)c(-c5ccc(F)c6sc(N)c(C#N)c56)c(F)c4n3)CC2)no1] |
| 74 | 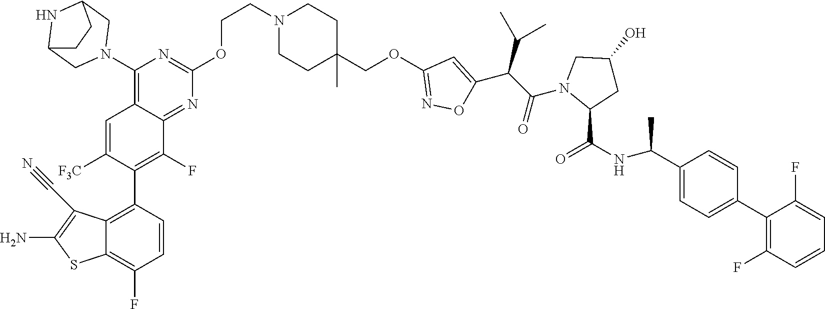 [SMILES: CC(C)[C@@H](C(=O)N1C[C@H](O)C[C@H]1C(=O)N[C@@H](C)c1ccc(-c2c(F)cccc2F)cc1)c1cc(OCC2(C)CCN(CCOc3nc(N4CC5CCC(C4)N5)c4cc(C(F)(F)F)c(-c5ccc(F)c6sc(N)c(C#N)c56)c(F)c4n3)CC2)no1] |
| 75 | 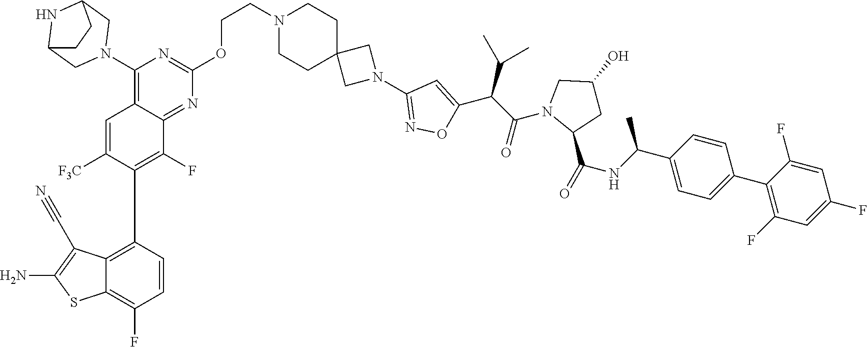 [SMILES: CC(C)[C@@H](C(=O)N1C[C@H](O)C[C@H]1C(=O)N[C@@H](C)c1ccc(-c2c(F)cc(F)cc2F)cc1)c1cc(N2CC3(CCN(CCOc4nc(N5CC6CCC(C5)N6)c5cc(C(F)(F)F)c(-c6ccc(F)c7sc(N)c(C#N)c67)c(F)c5n4)CC3)C2)no1] |
| 76 | 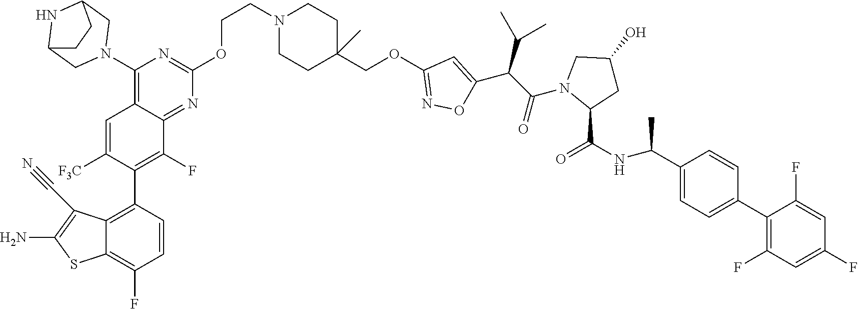 [SMILES: CC(C)[C@@H](C(=O)N1C[C@H](O)C[C@H]1C(=O)N[C@@H](C)c1ccc(-c2c(F)cc(F)cc2F)cc1)c1cc(OCC2(C)CCN(CCOc3nc(N4CC5CCC(C4)N5)c4cc(C(F)(F)F)c(-c5ccc(F)c6sc(N)c(C#N)c56)c(F)c4n3)CC2)no1] |
| 77 | 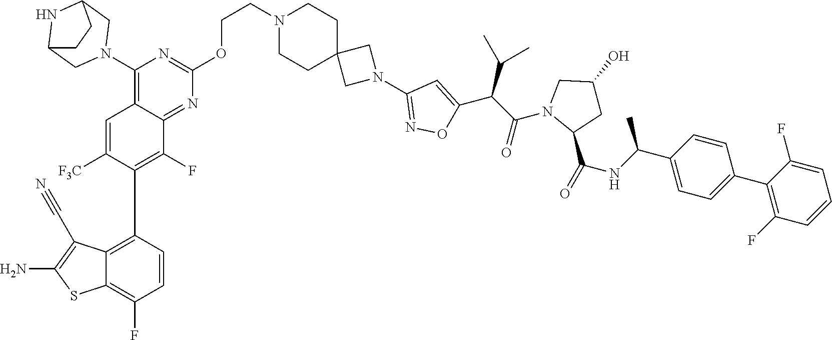 [SMILES: CC(C)[C@@H](C(=O)N1C[C@H](O)C[C@H]1C(=O)N[C@@H](C)c1ccc(-c2c(F)cccc2F)cc1)c1cc(N2CC3(CCN(CCOc4nc(N5CC6CCC(C5)N6)c5cc(C(F)(F)F)c(-c6ccc(F)c7sc(N)c(C#N)c67)c(F)c5n4)CC3)C2)no1] |
| 78 | 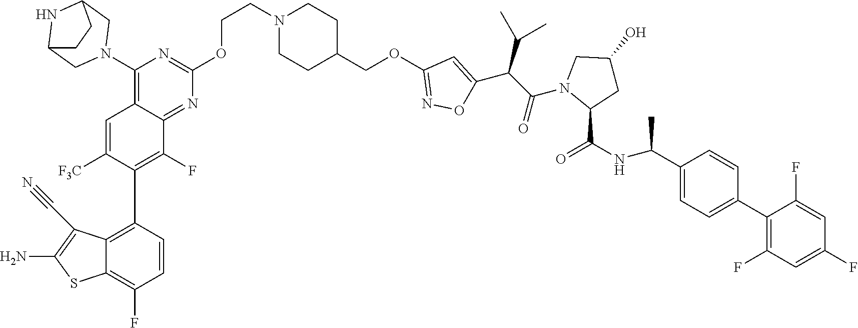 [SMILES: CC(C)[C@@H](C(=O)N1C[C@H](O)C[C@H]1C(=O)N[C@@H](C)c1ccc(-c2c(F)cc(F)cc2F)cc1)c1cc(OCC2CCN(CCOc3nc(N4CC5CCC(C4)N5)c4cc(C(F)(F)F)c(-c5ccc(F)c6sc(N)c(C#N)c56)c(F)c4n3)CC2)no1] |
| 79 | 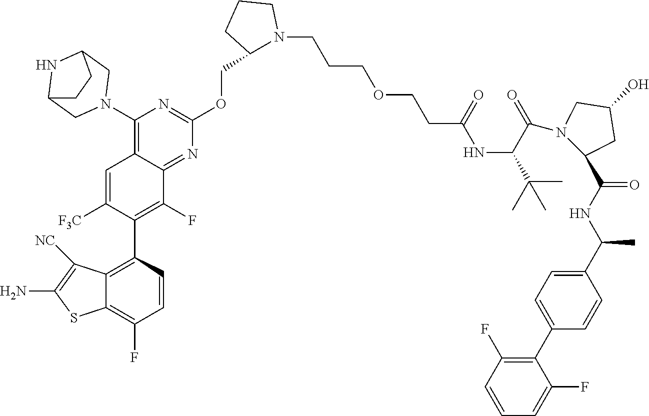 |
| 80 | 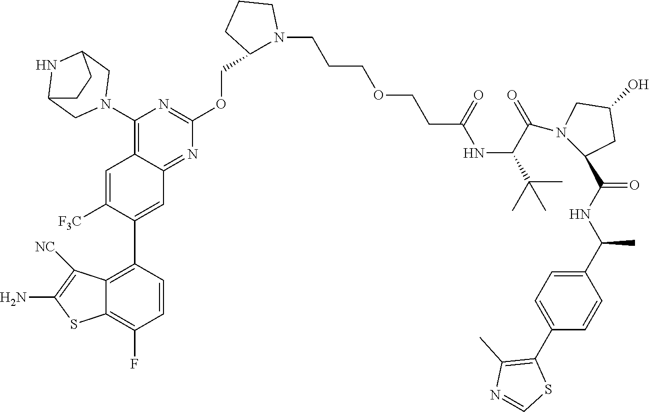 [SMILES: Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cc(C(F)(F)F)c(-c4ccc(F)c5sc(N)c(C#N)c45)cc3n2)C(C)(C)C)cc1] |
| 81 | 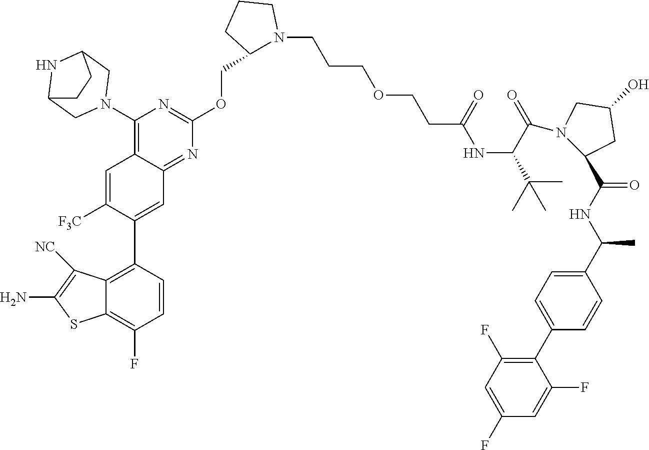 [SMILES: C[C@H](NC(=O)[C@@H]1C[C@@H](O)CN1C(=O)[C@@H](NC(=O)CCOCCCN1CCC[C@H]1COc1nc(N2CC3CCC(C2)N3)c2cc(C(F)(F)F)c(-c3ccc(F)c4sc(N)c(C#N)c34)cc2n1)C(C)(C)C)c1ccc(-c2c(F)cc(F)cc2F)cc1] |
| 82 | 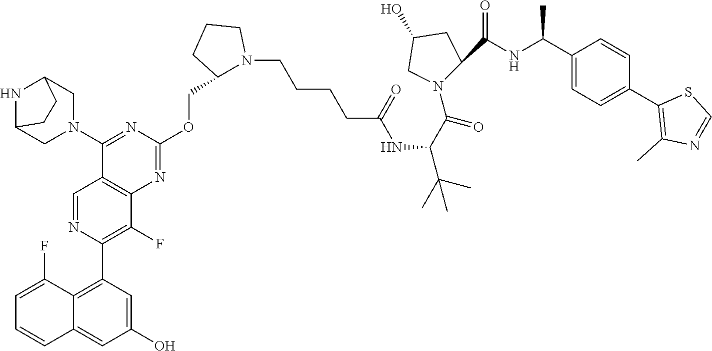 [SMILES: Cc1ncsc1-c1ccc([C@H](C)NC(=O)[C@@H]2C[C@@H](O)CN2C(=O)[C@@H](NC(=O)CCCCN2CCC[C@H]2COc2nc(N3CC4CCC(C3)N4)c3cnc(-c4cc(O)cc5cccc(F)c45)c(F)c3n2)C(C)(C)C)cc1] |
| 83 | 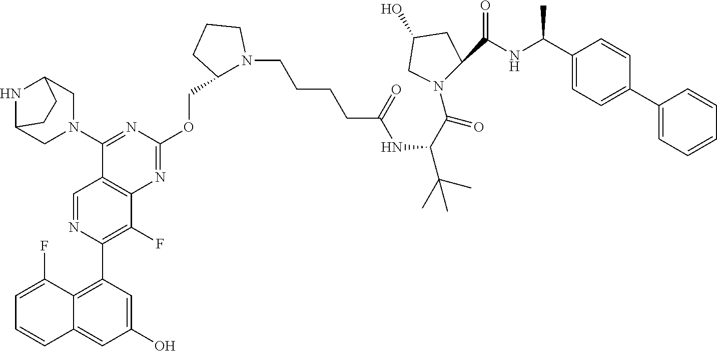 [SMILES: C[C@H](NC(=O)[C@@H]1C[C@@H](O)CN1C(=O)[C@@H](NC(=O)CCCCN1CCC[C@H]1COc1nc(N2CC3CCC(C2)N3)c2cnc(-c3cc(O)cc4cccc(F)c34)c(F)c2n1)C(C)(C)C)c1ccc(-c2ccccc2)cc1] |
| 84 | 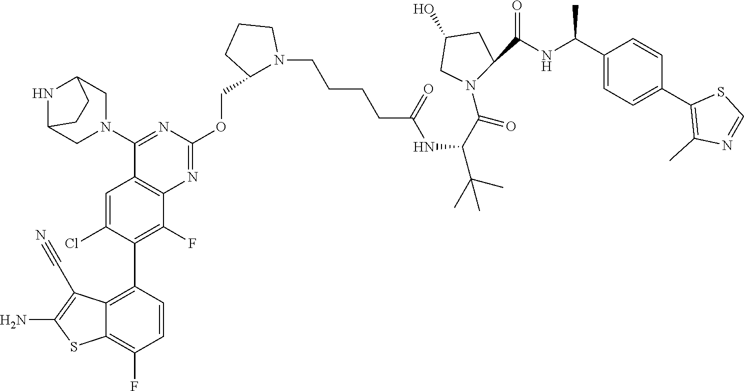 [SMILES: Cc1ncsc1-c1ccc([C@H](C)NC(=O)[C@@H]2C[C@@H](O)CN2C(=O)[C@@H](NC(=O)CCCCN2CCC[C@H]2COc2nc(N3CC4CCC(C3)N4)c3cc(Cl)c(-c4ccc(F)c5sc(N)c(C#N)c45)c(F)c3n2)C(C)(C)C)cc1] |
| 85 | 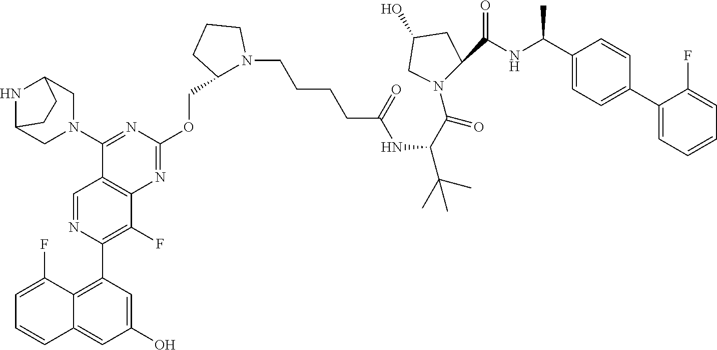 [SMILES: C[C@H](NC(=O)[C@@H]1C[C@@H](O)CN1C(=O)[C@@H](NC(=O)CCCCN1CCC[C@H]1COc1nc(N2CC3CCC(C2)N3)c2cnc(-c3cc(O)cc4cccc(F)c34)c(F)c2n1)C(C)(C)C)c1ccc(-c2ccccc2F)cc1] |
| 86 | 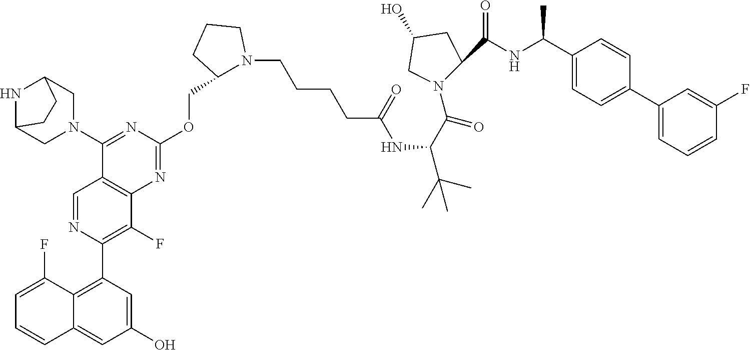 [SMILES: C[C@H](NC(=O)[C@@H]1C[C@@H](O)CN1C(=O)[C@@H](NC(=O)CCCCN1CCC[C@H]1COc1nc(N2CC3CCC(C2)N3)c2cnc(-c3cc(O)cc4cccc(F)c34)c(F)c2n1)C(C)(C)C)c1ccc(-c2cccc(F)c2)cc1] |
| 87 | 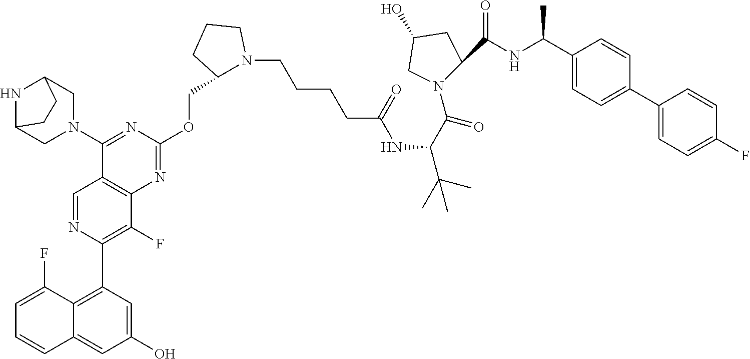 [SMILES: C[C@H](NC(=O)[C@@H]1C[C@@H](O)CN1C(=O)[C@@H](NC(=O)CCCCN1CCC[C@H]1COc1nc(N2CC3CCC(C2)N3)c2cnc(-c3cc(O)cc4cccc(F)c34)c(F)c2n1)C(C)(C)C)c1ccc(-c2ccc(F)cc2)cc1] |
| 88 | 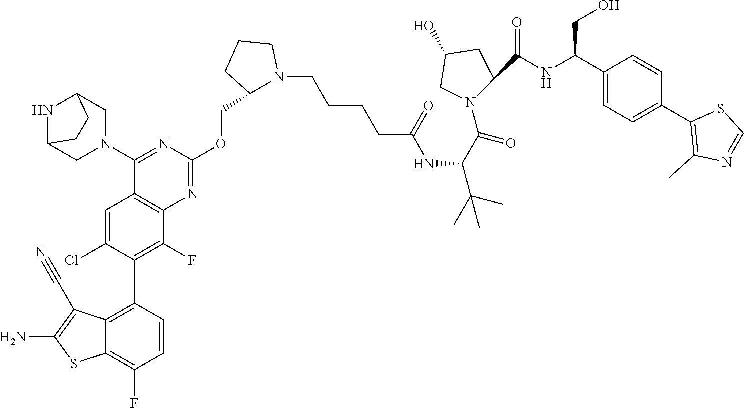 [SMILES: Cc1ncsc1-c1ccc([C@H](CO)NC(=O)[C@@H]2C[C@@H](O)CN2C(=O)[C@@H](NC(=O)CCCCN2CCC[C@H]2COc2nc(N3CC4CCC(C3)N4)c3cc(Cl)c(-c4ccc(F)c5sc(N)c(C#N)c45)c(F)c3n2)C(C)(C)C)cc1] |
| 89 | 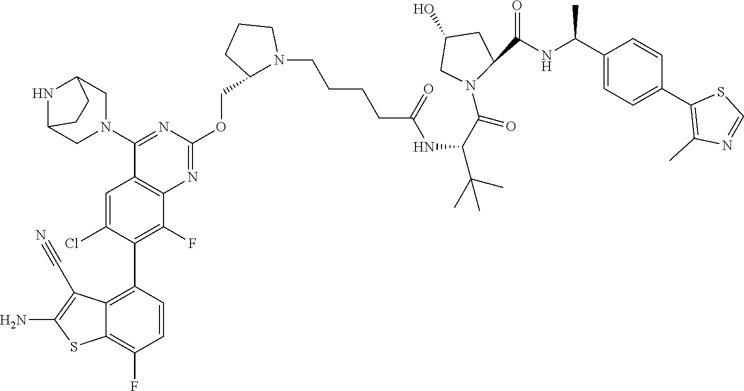 [SMILES: Cc1ncsc1-c1ccc([C@H](C)NC(=O)[C@@H]2C[C@@H](O)CN2C(=O)[C@@H](NC(=O)CCCCN2CCC[C@H]2COc2nc(N3CC4CCC(C3)N4)c3cc(Cl)c(-c4ccc(F)c5sc(N)c(C#N)c45)c(F)c3n2)C(C)(C)C)cc1] |
| 90 | 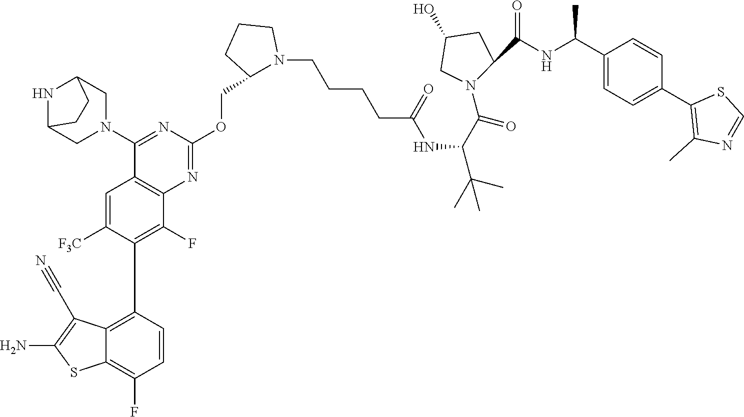 [SMILES: Cc1ncsc1-c1ccc([C@H](C)NC(=O)[C@@H]2C[C@@H](O)CN2C(=O)[C@@H](NC(=O)CCCCN2CCC[C@H]2COc2nc(N3CC4CCC(C3)N4)c3cc(C(F)(F)F)c(-c4ccc(F)c5sc(N)c(C#N)c45)c(F)c3n2)C(C)(C)C)cc1] |

| Ex | | | | LCMS |
|---|---|---|---|---|
| # | Method* | IUPAC Name | ¹H NMR | (ESI, m/z) |
| 40 | Ex. 14 | (2S,4R)-1-((2S)-2-(3-(3- | ¹H NMR (400 MHz, | 1103.5 |
| | | ((2S)-2-(((4-(3,8- | DMSO-d₆) δ: 10.21-10.06 | |
| | | diazabicyclo[3.2.1]octan- | (m, 1H), 9.03 (s, 1H), 8.41 | |
| | | 3-yl)-7-(8-ethynyl-7- | (d, J = 7.6 Hz, 1H), 7.99- | |
| | | fluoro-3- | 7.91 (m, 2H), 7.87-7.73 | |
| | | hydroxynaphthalen-1- | (m, 2H), 7.63-7.51 (m, | |
| | | yl)-8-fluoropyrido[4,3- | 4H), 7.48-7.37 (m, 4H), | |
| | | d]pyrimidin-2- | 7.17 (s, 1H), 5.14 (s, 1H), | |
| | | yl)oxy)methyl)pyrrolidin- | 4.96 (q, J = 7.2 Hz, 1H), | |
| | | 1- | 4.54-4.40 (m, 3H), 4.38- | |
| | | yl)propoxy)propanamido)- | 4.23 (m, 3H), 4.12-4.01 | |
| | | 3,3- | (m, 1H), 3.93 (d, J = 2.8 | |
| | | dimethylbutanoyl)-N- | Hz, 1H), 3.66-3.47 (m, | |
| | | ((S)-1-(2′-cyano-[1,1′- | 8H), 3.37 (d, J = 3.6 Hz, | |
| | | biphenyl]-4-yl)ethyl)-4- | 2H), 3.04 (dd, J = 3.2, 7.2 | |
| | | hydroxypyrrolidine-2- | Hz, 1H), 2.91-2.83 (m, | |
| | | carboxamide | 1H), 2.82-2.74 (m, 1H), | |
| | | | 2.30-2.22 (m, 2H), 2.16 | |
| | | | (d, J = 8.0 Hz, 1H), 2.03- | |
| | | | 1.97 (m, 1H), 1.90 (t, J = | |
| | | | 9.2 Hz, 1H), 1.83-1.78 | |
| | | | (m, 1H), 1.73-1.69 (m, | |
| | | | 2H), 1.65 (s, 4H), 1.66- | |
| | | | 1.63 (m, 1H), 1.38 (d, J = | |
| | | | 7.2 Hz, 3H), 1.23 (s, 3H), | |
| | | | 0.90 (d, J = 2.4 Hz, 9H), | |
| | | | 0.87-0.75 (m, 4H) | |
| 41 | Ex. 14 | (2S,4R)-1-((2S)-2-(3-(3- | ¹H NMR (400 MHz, | 1106.58 |
| | | ((2S)-2-(((4-(3,8- | DMSO-d₆) δ: 9.03 (s, 1H), | |
| | | diazabicyclo[3.2.1]octan- | 8.31 (d, J = 8.0 Hz, 1H), | |
| | | 3-yl)-7-(8-ethynyl-7- | 7.96 (dd, J = 6.0, 8.8 Hz, | |
| | | fluoro-3- | 1H), 7.85-7.80 (m, 1H), | |
| | | hydroxynaphthalen-1- | 7.45 (t, J = 8.8 Hz, 1H), | |
| | | yl)-8-fluoropyrido[4,3- | 7.39-7.34 (m, 3H), 7.18- | |
| | | d]pyrimidin-2- | 7.04 (m, 7H), 5.02-4.92 | |
| | | yl)oxy)methyl)pyrrolidin- | (m, 1H), 4.55-4.39 (m, | |
| | | 1- | 4H), 4.39-4.26 (m, 4H), | |
| | | yl)propoxy)propanamido)- | 4.10-4.03 (m, 1H), 3.91 | |
| | | 3,3- | (d, J = 3.2 Hz, 1H), 3.63 | |
| | | dimethylbutanoyl)-N- | (d, J = 11.2 Hz, 3H), 3.53 | |
| | | ((S)-1-(2′,6′-dimethyl- | (s, 5H), 3.38 (s, 3H), 3.06- | |
| | | [1,1′-biphenyl]-4- | 3.00 (m, 2H), 2.92-2.85 | |
| | | yl)ethyl)-4- | (m, 2H), 2.81-2.76 (m, | |
| | | hydroxypyrrolidine-2- | 2H), 2.33 (s, 2H), 2.23- | |
| | | carboxamide | 2.14 (m, 2H), 1.94 (s, 6H), | |
| | | | 1.65 (s, 8H), 1.40 (d, J = | |
| | | | 6.8 Hz, 3H), 0.91 (s, 9H) | |
| 42 | Ex. 14 | (2S,4R)-1-((2S)-2-(3-(3- | ¹H NMR (400 MHz, | 1130.1 |
| | | ((2S)-2-(((4-(3,8- | DMSO-d₆) δ: 10.28-10.20 | |
| | | diazabicyclo[3.2.1]octan- | (m, 1H), 9.10 (s, 1H), 8.40 | |
| | | 3-yl)-7-(8-ethynyl-7- | (d, J = 7.8 Hz, 1H), 8.00- | |
| | | fluoro-3- | 7.95 (m, 1H), 7.84 (d, J = | |
| | | hydroxynaphthalen-1- | 8.0 Hz, 1H), 7.48-7.43 | |
| | | yl)-8-fluoropyrido[4,3- | (m, 3H), 7.41 (d, J = 2.4 | |
| | | d]pyrimidin-2- | Hz, 2H), 7.38 (s, 1H), 7.36- | |
| | | yl)oxy)methyl)pyrrolidin- | 7.33 (m, 1H), 7.32-7.28 | |
| | | 1- | (m, 2H), 7.21 (d, J = 2.0 | |
| | | yl)propoxy)propanamido)- | Hz, 1H), 5.19-5.07 (m, | |
| | | 3,3- | 1H), 5.00-4.91 (m, 1H), | |
| | | dimethylbutanoyl)-N- | 4.64 (d, J = 13.2 Hz, 1H), | |
| | | ((S)-1-(2′-chloro-6′- | 4.53-4.48 (m, 2H), 4.44 | |
| | | fluoro-[1,1′-biphenyl]-4- | (d, J = 8.4 Hz, 2H), 4.28 | |
| | | yl)ethyl)-4- | (d, J = 1.6 Hz, 1H), 4.11 | |
| | | hydroxypyrrolidine-2- | (s, 2H), 3.95 (s, 1H), 3.93- | |
| | | carboxamide | 3.85 (m, 2H), 3.64-3.48 | |
| | | | (m, 6H), 3.40 (s, 2H), 3.30- | |
| | | | 3.28 (m, 2H), 2.34-2.31 | |
| | | | (m, 1H), 2.06-1.90 (m, | |
| | | | 8H), 1.85-1.69 (m, 7H), | |
| | | | 1.39 (d, J = 6.4 Hz, 3H), | |
| | | | 1.23 (s, 1H), 0.91 (d, J = | |
| | | | 3.2 Hz, 9H) | |
| 43 | Ex. 14 | (2S,4R)-1-((2S)-2-(3-(3- | ¹H NMR (400 MHz, | 1164.5 |
| | | ((2S)-2-(((4-(3,8- | DMSO-d₆) δ: 10.21 (s, | |
| | | diazabicyclo[3.2.1]octan- | 1H), 9.13-9.03 (m, 1H), | |
| | | 3-yl)-7-(8-ethynyl-7- | 8.39 (d, J = 8.0 Hz, 1H), | |
| | | fluoro-3- | 7.98 (dd, J = 6.0, 9.2 Hz, | |
| | | hydroxynaphthalen-1- | 1H), 7.84 (d, J = 7.6 Hz, | |
| | | yl)-8-fluoropyrido[4,3- | 1H), 7.73-7.62 (m, 3H), | |
| | | d]pyrimidin-2- | 7.50-7.36 (m, 4H), 7.27- | |
| | | yl)oxy)methyl)pyrrolidin- | 7.18 (m, 3H), 5.36-5.04 | |
| | | 1- | (m, 1H), 5.02-4.94 (m, | |
| | | yl)propoxy)propanamido)- | 1H), 4.64-4.49 (m, 2H), | |
| | | 3,3- | 4.48-4.34 (m, 3H), 4.29 | |
| | | dimethylbutanoyl)-N- | (s, 1H), 4.21-4.10 (m, | |
| | | ((S)-1-(2′-fluoro-6′- | 1H), 4.01-3.86 (m, 3H), | |
| | | (trifluoromethyl)-[1,1′- | 3.85-3.70 (m, 2H), 3.67- | |
| | | biphenyl]-4-yl)ethyl)-4- | 3.47 (m, 5H), 3.40 (s, 1H), | |
| | | hydroxypyrrolidine-2- | 3.14-3.01 (m, 1H), 2.99- | |
| | | carboxamide | 2.82 (m, 2H), 2.30-2.25 | |
| | | | (m, 1H), 2.10-1.88 (m, | |
| | | | 4H), 1.88-1.78 (m, 5H), | |
| | | | 1.70 (d, J = 7.2, 13.6 Hz, | |
| | | | 6H), 1.40 (d, J = 6.8 Hz, | |
| | | | 3H), 1.24 (s, 1H), 0.91 (d, | |
| | | | J = 3.2 Hz, 9H) | |
| 44 | Ex. 14 | (2S,4R)-1-((2S)-2-(3-(3- | ¹H NMR (400 MHz, | 1131.5 |
| | | ((2S)-2-(((4-(3,8- | DMSO-d₆) δ: 10.22 (s, | |
| | | diazabicyclo[3.2.1]octan- | 1H), 9.09 (s, 1H), 8.41 (d, | |
| | | 3-yl)-7-(8-ethynyl-7- | J = 7.6 Hz, 1H), 8.15 (s, | |
| | | fluoro-3- | 1H), 7.99 (dd, J = 6.0, 9.2 | |
| | | hydroxynaphthalen-1- | Hz, 1H), 7.88-7.80 (m, | |
| | | yl)-8-fluoropyrido[4,3- | 1H), 7.59-7.47 (m, 2H), | |
| | | d]pyrimidin-2- | 7.45-7.41 (m, 5H), 7.30- | |
| | | yl)oxy)methyl)pyrrolidin- | 7.20 (m, 2H), 5.23-5.02 | |
| | | 1- | (m, 1H), 5.00-4.89 (m, | |
| | | yl)propoxy)propanamido)- | 1H), 4.62 (d, J = 12.8 Hz, | |
| | | 3,3- | 1H), 4.55-4.50 (m, 1H), | |
| | | dimethylbutanoyl)-4- | 4.47-4.38 (m, 3H), 4.28 | |
| | | hydroxy-N-((S)-1- | (d, J = 2.4 Hz, 1H), 4.24- | |
| | | (2′,3′,6′-trifluoro-[1,1′- | 4.11 (m, 1H), 4.01 (s, 2H), | |
| | | biphenyl]-4- | 3.97-3.93 (m, 1H), 3.88- | |
| | | yl)ethyl)pyrrolidine-2- | 3.77 (m, 2H), 3.65-3.46 | |
| | | carboxamide | (m, 5H), 3.13-3.05 (m, | |
| | | | 1H), 3.01-2.88 (m, 2H), | |
| | | | 2.31-2.24 (m, 2H), 2.07- | |
| | | | 1.86 (m, 8H), 1.85-1.66 | |
| | | | (m, 7H), 1.39 (d, J = 6.8 | |
| | | | Hz, 3H), 1.24 (s, 1H), 0.91 | |
| | | | (d, J = 3.2 Hz, 9H) | |
| 45 | Ex. 14 | (2S,4R)-1-((2S)-2-(3-(3- | ¹H NMR (400 MHz, | 1146.4 |
| | | ((2S)-2-(((4-(3,8- | DMSO-d₆) δ: 9.04 (s, 1H), | |
| | | diazabicyclo[3.2.1]octan- | 8.37 (d, J = 8.4 Hz, 1H), | |
| | | 3-yl)-7-(8-ethynyl-7- | 8.33-8.26 (m, 2H), 8.00- | |
| | | fluoro-3- | 7.95 (m, 1H), 7.87-7.80 | |
| | | hydroxynaphthalen-1- | (m, 1H), 7.60-7.58 (m, | |
| | | yl)-8-fluoropyrido[4,3- | 1H), 7.57-7.55 (m, 1H), | |
| | | d]pyrimidin-2- | 7.49-7.43 (m, 2H), 7.42- | |
| | | yl)oxy)methyl)pyrrolidin- | 7.34 (m, 4H), 7.22 (s, 1H), | |
| | | 1- | 7.20-7.16 (m, 2H), 4.99 | |
| | | yl)propoxy)propanamido)- | (m, 1H), 4.55-4.48 (m, | |
| | | 3,3- | 2H), 4.47-4.40 (m, 2H), | |
| | | dimethylbutanoyl)-N- | 4.40-4.32 (m, 2H), 4.30 | |
| | | ((S)-1-(2′,6′-dichloro- | (s, 2H), 4.12-4.04 (m, | |
| | | [1,1′-bipheny]]-4- | 1H), 3.93 (d, J = 2.4 Hz, | |
| | | yl)ethyl)-4- | 1H), 3.68-3.60 (m, 4H), | |
| | | hydroxypyrrolidine-2- | 3.59-3.51 (m, 8H), 3.09- | |
| | | carboxamide | 3.00 (m, 2H), 2.97-2.75 | |
| | | | (m, 3H), 1.75-1.55 (m, | |
| | | | 12H), 1.40 (d, J = 6.8 Hz, | |
| | | | 3H), 0.92 (d, J = 2.8 Hz, | |
| | | | 9H) | |
| 46 | Ex. 14 | (2S,4R)-1-((2S)-2-(3-(3- | ¹H NMR (400 MHz, | 1114.5 |
| | | ((2S)-2-(((4-(3,8- | DMSO-d₆) δ: 9.05 (s, 1H), | |
| | | diazabicyclo[3.2.1]octan- | 8.20 (s, 1H), 8.17-8.11 | |
| | | 3-yl)-7-(8-ethynyl-7- | (m, 1H), 8.01-7.95 (m, | |
| | | fluoro-3- | 1H), 7.86-7.78 (m, 1H), | |
| | | hydroxynaphthalen-1- | 7.47 (s, 1H), 7.40 (d, J = | |
| | | yl)-8-fluoropyrido[4,3- | 2.4 Hz, 1H), 7.30 (s, 1H), | |
| | | d]pyrimidin-2- | 7.19 (d, J = 2.4 Hz, 1H), | |
| | | yl)oxy)methyl)pyrrolidin- | 7.08 (d, J = 8.4 Hz, 2H), | |
| | | 1- | 7.02 (s, 1H), 6.41 (d, J = | |
| | | yl)propoxy)propanamido)- | 8.4 Hz, 2H), 4.76 (quin, J = | |
| | | 3,3- | 7.2 Hz, 1H), 4.52 (br d, J = | |
| | | dimethylbutanoyl)-N- | 9.6 Hz, 2H), 4.43-4.29 | |
| | | ((S)-1-(4-((S)-2- | (m, 4H), 4.26 (br s, 1H), | |
| | | carbamoylpyrrolidin-1- | 4.13-4.06 (m, 1H), 3.93 | |
| | | yl)phenyl)ethyl)-4- | (d, J = 3.2 Hz, 1H), 3.86- | |
| | | hydroxypyrrolidine-2- | 3.79 (m, 2H), 3.54 (br d, J = | |
| | | carboxamide | 9.2 Hz, 10H), 3.38 (br d, | |
| | | | J = 3.2 Hz, 3H), 3.18-3.12 | |
| | | | (m, 1H), 3.09-3.03 (m, | |
| | | | 1H), 2.87 (br s, 2H), 2.32- | |
| | | | 2.11 (m, 5H), 2.03-1.87 | |
| | | | (m, 6H), 1.72-1.59 (m, | |
| | | | 7H), 1.28 (d, J = 6.8 Hz, | |
| | | | 3H), 0.94-0.88 (m, 9H) | |
| 47 | Ex. 14 | (2S,4R)-1-((2S)-2-(3-(3- | ¹H NMR (400 MHz, | 1107.5 |
| | | ((2S)-2-(((4-(3,8- | DMSO-d₆) δ: 9.05 (s, 1H), | |
| | | diazabicyclo[3.2.1]octan- | 8.34 (br d, J = 8.4 Hz, 1H), | |
| | | 3-yl)-7-(8-ethynyl-7- | 8.28 (d, J = 5.2 Hz, 1H), | |
| | | fluoro-3- | 8.20 (s, 2H), 7.97 (dd, J = | |
| | | hydroxynaphthalen-1- | 6.0, 9.2 Hz, 1H), 7.83 (dd, | |
| | | yl)-8-fluoropyrido[4,3- | J = 1.6, 9.2 Hz, 1H), 7.45 | |
| | | d]pyrimidin-2- | (t, J = 9.2 Hz, 1H), 7.41- | |
| | | yl)oxy)methyl)pyrrolidin- | 7.38 (m, 2H), 7.37 (s, 1H), | |
| | | 1- | 7.20-7.09 (m, 5H), 5.06- | |
| | | yl)propoxy)propanamido)- | 4.89 (m, 2H), 4.57-4.49 | |
| | | 3,3- | (m, 3H), 4.47-4.31 (m, | |
| | | dimethylbutanoyl)-N- | 6H), 4.30-4.25 (m, 2H), | |
| | | ((S)-1-(4-(2,4- | 4.15-4.08 (m, 3H), 3.52- | |
| | | dimethylpyridin-3- | 3.48 (m, 2H), 3.40-3.34 | |
| | | yl)phenyl)ethyl)-4- | (m, 3H), 3.10-3.00 (m, | |
| | | hydroxypyrrolidine-2- | 2H), 2.94-2.79 (m, 3H), | |
| | | carboxamide | 2.42-2.32 (m, 2H), 2.28- | |
| | | | 2.19 (m, 2H), 2.15 (s, 3H), | |
| | | | 2.01-1.96 (m, 4H), 1.93- | |
| | | | 1.87 (m, 1H), 1.82 (dt, J = | |
| | | | 4.4, 8.4 Hz, 2H), 1.74- | |
| | | | 1.65 (m, 5H), 1.40 (d, J = | |
| | | | 6.8 Hz, 3H), 0.90 (br d, J = | |
| | | | 2.4 Hz, 10H) | |
| 48 | Ex. 14 | (2S,4R)-1-((2S)-2-(3-(3- | ¹H NMR (400 MHz, | 1147.4 |
| | | ((2S)-2-(((4-(3,8- | DMSO-d₆) δ: 10.23-10.05 | |
| | | diazabicyclo[3.2.1]octan- | (m, 1H), 9.06-9.01 (m, | |
| | | 3-yl)-7-(8-ethynyl-7- | 1H), 8.76 (d, J = 4.0 Hz, | |
| | | fluoro-3- | 1H), 8.37 (d, J = 8.0 Hz, | |
| | | hydroxynaphthalen-1- | 1H), 7.97 (dd, J = 6.0, 9.2 | |
| | | yl)-8-fluoropyrido[4,3- | Hz, 1H), 7.90 (d, J = 7.6 | |
| | | d]pyrimidin-2- | Hz, 1H), 7.85-7.75 (m, | |
| | | yl)oxy)methyl)pyrrolidin- | 2H), 7.46 (d, 8.8 Hz, 1H), | |
| | | 1- | 7.39 (dd, J = 2.8, 5.6 Hz, | |
| | | yl)propoxy)propanamido)- | 3H), 7.30 (d, J = 8.0 Hz, | |
| | | 3,3- | 2H), 7.18 (d, J = 2.0 Hz, | |
| | | dimethylbutanoyl)-4- | 1H), 5.00-4.94 (m, 1H), | |
| | | hydroxy-N-((S)-1-(4-(2- | 4.55-4.47 (m, 2H), 4.43 | |
| | | (trifluoromethyl)pyridin- | (m, 1H), 4.39-4.31 (m, | |
| | | 3- | 2H), 4.29 (s, 2H), 4.12- | |
| | | yl)phenyl)ethyl)pyrrolidine- | 4.04 (m, 1H), 3.92 (d, J = | |
| | | 2-carboxamide | 2.4 Hz, 1H), 3.66-3.48 | |
| | | | (m, 10H), 3.10-2.97 (m, | |
| | | | 2H), 2.94-2.79 (m, 3H), | |
| | | | 2.41-2.13 (m, 5H), 2.04- | |
| | | | 1.97 (m, 1H), 1.96-1.85 | |
| | | | (m, 2H), 1.82-1.76 (m, | |
| | | | 1H), 1.76-1.54 (m, 11H), | |
| | | | 1.39 (d, J = 6.8 Hz, 3H), | |
| | | | 0.91 (d, J = 2.0 Hz, 9H) | |
| 49 | Ex. 14 | (2S,4R)-1-((2S)-2-(3-(3- | ¹H NMR (400 MHz, | 1132.6 |
| | | ((2S)-2-(((4-(3,8- | DMSO-d₆) δ: = 9.10 (s, | |
| | | diazabicyclo[3.2.1]octan- | 1H), 8.38 (d, J = 7.6 Hz, | |
| | | 3-yl)-7-(8-ethynyl-7- | 1H), 8.16 (s, 1H), 7.98 (dd, | |
| | | fluoro-3- | J = 6.0, 9.2 Hz, 1H), 7.83 | |
| | | hydroxynaphthalen-1- | (dd, J = 1.6, 9.2 Hz, 1H), | |
| | | yl)-8-fluoropyrido[4,3- | 7.47 (t, J = 8.8 Hz, 1H), | |
| | | d]pyrimidin-2- | 7.42-7.34 (m, 6H), 7.34- | |
| | | yl)oxy)methyl)pyrrolidin- | 7.29 (m, 2H), 7.19 (d, J = | |
| | | 1- | 2.4 Hz, 1H), 4.93 (br t, J = | |
| | | yl)propoxy)propanamido)- | 7.2 Hz, 1H), 4.63 (br d, J = | |
| | | 3,3- | 12.8 Hz, 1H), 4.54-4.49 | |
| | | dimethylbutanoyl)-4- | (m, 1H), 4.48-4.44 (m, | |
| | | hydroxy-N-((S)-1- | 1H), 4.43-4.37 (m, 2H), | |
| | | (2′,4′,6′-trifluoro-[1,1′- | 4.31-4.25 (m, 1H), 4.23- | |
| | | biphenyl]-4- | 4.14 (m, 1H), 4.04 (br s, | |
| | | yl)ethyl)pyrrolidine-2- | 2H), 3.94 (d, J = 1.6 Hz, | |
| | | carboxamide | 1H), 3.78 (br t, J = 14.4 | |
| | | | Hz, 3H), 3.63-3.53 (m, | |
| | | | 4H), 3.52-3.47 (m, 2H), | |
| | | | 3.41-3.34 (m, 3H), 3.14- | |
| | | | 3.08 (m, 1H), 2.99-2.92 | |
| | | | (m, 2H), 2.30-2.24 (m, | |
| | | | 1H), 2.05-1.94 (m, 2H), | |
| | | | 1.92-1.87 (m, 4H), 1.84- | |
| | | | 1.64 (m, 7H), 1.41-1.35 | |
| | | | (m, 3H), 0.90 (d, J = 3.2 | |
| | | | Hz, 9H) | |
| 50 | Ex. 14 | (S)-1-((2S,4R)-4- | ¹H NMR (400 MHz, | 1160.5 |
| | | hydroxy-2-(((S)-1-(2′- | DMSO-d₆) δ: 10.29-10.01 | |
| | | methyl-6′- | (m, 1H), 9.03 (s, 1H), 8.35 | |
| | | (trifluoromethyl)-[1,1′- | (d, J = 8.4 Hz, 1H), 8.02- | |
| | | biphenyl]-4- | 7.92 (m, 1H), 7.88-7.80 | |
| | | yl)ethyl)carbamoyl) | (m, 1H), 7.68-7.56 (m, | |
| | | pyrrolidin-1-yl)-3,3- | 2H), 7.52-7.42 (m, 2H), | |
| | | dimethyl-1-oxobutan-2- | 7.40-7.33 (m, 3H), 7.18 | |
| | | yl 3-(3-((2S)-2-(((4- | (d, J = 2.4 Hz, 1H), 7.10 | |
| | | (3,8- | (d, J = 8.0 Hz, 2H), 5.25- | |
| | | diazabicyclo[3.2.1]octan- | 4.93 (m, 2H), 4.53-4.39 | |
| | | 3-yl)-7-(8-ethynyl-7- | (m, 3H), 4.37-4.26 (m, | |
| | | fluoro-3- | 3H), 4.10-4.02 (m, 1H), | |
| | | hydroxynaphthalen-1- | 3.93 (d, J = 2.8 Hz, 1H), | |
| | | yl)-8-fluoropyrido[4,3- | 3.68-3.61 (m, 2H), 3.56 | |
| | | d]pyrimidin-2- | (s, 4H), 3.52-3.48 (m, | |
| | | yl)oxy)methyl)pyrrolidin- | 1H), 3.38 (s, 1H), 3.06- | |
| | | 1- | 3.00 (m, 1H), 2.89-2.76 | |
| | | yl)propoxy)propanoate | (m, 2H), 2.26-2.15 (m, | |
| | | | 2H), 1.95 (s, 3H), 1.85- | |
| | | | 1.80 (m, 1H), 1.66 (s, 7H), | |
| | | | 1.40 (d, J = 6.8 Hz, 3H), | |
| | | | 1.23 (s, 3H), 1.17-1.15 | |
| | | | (m, 2H), 0.91 (d, J = 2.0 | |
| | | | Hz, 11H) | |
| 51 | Ex. 14 | (2S,4R)-1-((2S)-2-(3-(3- | ¹H NMR (400 MHz, | 1113.6 |
| | | ((2S)-2-(((4-(3,8- | DMSO-d₆) δ: 9.04 (s, 1H), | |
| | | diazabicyclo[3.2.1]octan- | 8.43-8.33 (m, 1H), 8.19 | |
| | | 3-yl)-7-(8-ethynyl-7- | (s, 1H), 8.05-7.92 (m, | |
| | | fluoro-3- | 1H), 7.88-7.76 (m, 1H), | |
| | | hydroxynaphthalen-1- | 7.65-7.15 (m, 11H), 4.98- | |
| | | yl)-8-fluoropyrido[4,3- | 4.88 (m, 1H), 4.55-4.47 | |
| | | d]pyrimidin-2- | (m, 2H), 4.45-4.23 (m, | |
| | | yl)oxy)methyl)pyrrolidin- | 5H), 4.16-4.00 (m, 2H), | |
| | | 1- | 3.92 (d, J = 2.4 Hz, 1H), | |
| | | yl)propoxy)propanamido)- | 3.62-3.54 (m, 7H), 3.38 | |
| | | 3,3- | (s, 3H), 3.14-2.99 (m, | |
| | | dimethylbutanoyl)-N- | 7H), 2.84 (d, J = 6.4 Hz, | |
| | | ((S)-1-(2′,3′-difluoro- | 2H), 1.74-1.58 (m, 9H), | |
| | | [1,1′-biphenyl]-4- | 1.37 (d, J = 7.2 Hz, 3H), | |
| | | yl)ethyl)-4- | 0.93-0.87 (m, 9H) | |
| | | hydroxypyrrolidine-2- | | |
| | | carboxamide | | |
| 52 | Ex. 14 | (2S,4R)-1-((2S)-2-(3-(3- | ¹H NMR (400 MHz, | 1132.3 |
| | | ((2S)-2-(((4-(3,8- | DMSO-d₆) δ: 9.03 (s, 1H), | |
| | | diazabicyclo[3.2.1]octan- | 8.38 (d, J = 7.6 Hz, 1H), | |
| | | 3-yl)-7-(8-ethynyl-7- | 8.22 (s, 1H), 8.01-7.94 | |
| | | fluoro-3- | (m, 1H), 7.86-7.77 (m, | |
| | | hydroxynaphthalen-1- | 1H), 7.71-7.61 (m, 4H), | |
| | | yl)-8-fluoropyrido[4,3- | 7.46 (t, J = 9.2 Hz, 1H), | |
| | | d]pyrimidin-2- | 7.41-7.33 (m, 3H), 7.17 | |
| | | yl)oxy)methyl)pyrrolidin- | (d, J = 2.4 Hz, 1H), 4.91 (t, | |
| | | 1- | J = 6.8 Hz, 1H), 4.54-4.45 | |
| | | yl)propoxy)propanamido)- | (m, 2H), 4.43-4.24 (m, | |
| | | 3,3- | 4H), 4.11-4.01 (m, 1H), | |
| | | dimethylbutanoyl)-4- | 3.92 (d, J = 2.8 Hz, 1H), | |
| | | hydroxy-N-((S)-1- | 3.67-3.51 (m, 8H), 3.43- | |
| | | (3′,4′,5′-trifluoro-[1,1′- | 3.34 (m, 8H), 3.07-3.02 | |
| | | biphenyl]-4- | (m, 1H), 2.92-2.76 (m, | |
| | | yl)ethyl)pyrrolidine-2- | 2H), 2.21-2.14 (m, 1H), | |
| | | carboxamide | 2.01-1.87 (m, 2H), 1.66 | |
| | | | (s, 9H), 1.35 (d, J = 7.2 Hz, | |
| | | | 3H), 0.90 (d, J = 2.4 Hz, | |
| | | | 9H) | |
| 53 | Ex. 14 | (2S,4R)-1-((2S)-2-(2-(3- | ¹H NMR (400 MHz, | 1118.3 |
| | | ((2S)-2-(((4-(3,8- | DMSO-d₆) δ: 10.16 (s, | |
| | | diazabicyclo[3.2.1]octan- | 1H), 9.05 (s, 1H), 8.48- | |
| | | 3-yl)-7-(8-ethynyl-7- | 8.42 (m, 1H), 7.97 (dd, J = | |
| | | fluoro-3- | 6.0, 9.2 Hz, 1H), 7.54 (br | |
| | | hydroxynaphthalen-1- | dd, J = 4.8, 9.2 Hz, 1H), | |
| | | yl)-8-fluoropyrido[4,3- | 7.48-7.44 (m, 1H), 7.42 | |
| | | d]pyrimidin-2- | (s, 4H), 7.40 (br s, 1H), | |
| | | yl)oxy)methyl)pyrrolidin- | 7.33-7.23 (m, 2H), 7.20- | |
| | | 1- | 7.16 (m, 1H), 5.34-5.05 | |
| | | yl)propoxy)acetamido)- | (m, 1H), 4.93 (br t, J = 7.2 | |
| | | 3,3-dimethylbutanoyl)- | Hz, 1H), 4.56-4.49 (m, | |
| | | 4-hydroxy-N-((S)-1- | 2H), 4.39-4.25 (m, 3H), | |
| | | (2′,3′,6′-trifluoro-[1,1′- | 4.15-4.06 (m, 1H), 3.94 | |
| | | biphenyl]-4- | (s, 1H), 3.73-3.53 (m, | |
| | | yl)ethyl)pyrrolidine-2- | 8H), 3.12-3.05 (m, 1H), | |
| | | carboxamide | 2.95-2.82 (m, 2H), 2.27- | |
| | | | 2.18 (m, 2H), 2.10-2.00 | |
| | | | (m, 2H), 1.74 (br s, 4H), | |
| | | | 1.38 (br d, J = 6.8 Hz, 3H), | |
| | | | 1.17 (br s, 6H), 0.90 (s, | |
| | | | 9H), 0.82-0.73 (m, 4H) | |
| 54 | Ex. 14 | (2S,4R)-1-((2S)-2-(3-(3- | ¹H NMR (400 MHz, | |
| | | ((2S)-2-(((4-(3,8- | DMSO-d₆) δ: 10.17 (d, J = | |
| | | diazabicyclo[3.2.1]octan- | 3.2 Hz, 1H), 9.08 (s, 1H), | |
| | | 3-yl)-7-(8-ethynyl-7- | 8.39 (d, J = 7.2 Hz, 1H), | |
| | | fluoro-3- | 8.14 (s, 1H), 8.01-7.86 | |
| | | hydroxynaphthalen-1- | (m, 2H), 7.82 (d, J = 8.8 | 1150.6 |
| | | yl)-8-fluoropyrido[4,3- | Hz, 1H), 7.46 (s, 1H), 7.46- | |
| | | d]pyrimidin-2- | 7.42 (m, 4H), 7.41-7.38 | |
| | | yl)oxy)methyl)pyrrolidin- | (m, 1H), 7.18 (d, J = 2.4 | |
| | | 1- | Hz, 1H), 4.94 (t, J = 7.2 | |
| | | yl)propoxy)propanamido)- | Hz, 1H), 4.60 (d, J = 12.8 | |
| | | 3,3- | Hz, 1H), 4.51 (d, J = 8.8 | |
| | | dimethylbutanoyl)-4- | Hz, 1H), 4.45-4.36 (m, | |
| | | hydroxy-N-((S)-1- | 3H), 4.27 (s, 1H), 4.21- | |
| | | (2′,3′,5′,6′-tetrafluoro- | 4.09 (m, 1H), 3.93 (d, J = | |
| | | [1,1′-biphenyl]-4- | 2.8 Hz, 3H), 3.80-3.67 | |
| | | yl)ethyl)pyrrolidine-2- | (m, 2H), 3.61-3.48 (m, | |
| | | carboxamide | 4H), 3.42-3.37 (m, 4H), | |
| | | | 3.11-3.04 (m, 1H), 2.97- | |
| | | | 2.82 (m, 2H), 2.30-2.23 | |
| | | | (m, 2H), 2.00 (d, J = 7.2 | |
| | | | Hz, 2H), 1.89-1.82 (m, | |
| | | | 4H), 1.77-1.60 (m, 6H), | |
| | | | 1.38 (d, J = 6.8 Hz, 3H), | |
| | | | 1.23 (s, 1H), 0.90 (d, J = | |
| | | | 3.2 Hz, 9H) | |
| 55 | Ex. 14 | (2S,4R)-1-((2S)-2-(3-(3- | ¹H NMR (400 MHz, | 1131.5 |
| | | ((2S)-2-(((4-(3,8- | DMSO-d₆) δ: 9.03 (s, 1H), | |
| | | diazabicyclo[3.2.1]octan- | 8.38 (d, J = 8.8 Hz, 1H), | |
| | | 3-yl)-7-(8-ethynyl-7- | 8.19 (s, 1H), 7.97 (dd, J = | |
| | | fluoro-3- | 5.6, 9.6 Hz, 1H), 7.86- | |
| | | hydroxynaphthalen-1- | 7.77 (m, 1H), 7.50-7.42 | |
| | | yl)-8-fluoropyrido[4,3- | (m, 4H), 7.42-7.35 (m, | |
| | | d]pyrimidin-2- | 5H), 7.17 (d, J = 2.4 Hz, | 1120.4 |
| | | yl)oxy)methyl)pyrrolidin- | 1H), 4.97-4.88 (m, 1H), | |
| | | 1- | 4.54-4.45 (m, 2H), 4.44- | |
| | | yl)propoxy)propanamido)- | 4.38 (m, 1H), 4.38-4.24 | |
| | | 3,3- | (m, 3H), 4.13-4.02 (m, | |
| | | dimethylbutanoyl)-4- | 1H), 3.92 (d, J = 3.2 Hz, | |
| | | hydroxy-N-((S)-1- | 1H), 3.70-3.61 (m, 2H), | |
| | | (2′,3′,4′-trifluoro-[1,1′- | 3.57 (s, 6H), 3.09-2.98 | |
| | | biphenyl]-4- | (m, 2H), 2.91-2.84 (m, | |
| | | yl)ethyl)pyrrolidine-2- | 2H), 2.81-2.77 (m, 2H), | |
| | | carboxamide | 2.18 (d, J = 8.0 Hz, 2H), | |
| | | | 2.06-1.95 (m, 2H), 1.94- | |
| | | | 1.84 (m, 2H), 1.83-1.75 | |
| | | | (m, 2H), 1.67 (s, 6H), 1.37 | |
| | | | (d, J = 7.2 Hz, 3H), 0.95- | |
| | | | 0.88 (m, 11H) | |
| 56 | Ex. 14 | (2S,4R)-1-((2S)-2-(3-(3- | ¹H NMR (400 MHz, | |
| | | ((2S)-2-(((4-(3,8- | DMSO-d₆) δ: 9.46 (s, 1H), | |
| | | diazabicyclo[3.2.1]octan- | 8.79-8.71 (m, 1H), 8.61 | |
| | | 3-yl)-7-(8-ethynyl-7- | (br d, J = 3.2 Hz, 1H), 8.42- | |
| | | fluoro-3- | 8.33 (m, 1H), 8.28-8.21 | |
| | | hydroxynaphthalen-1- | (m, 1H), 7.93-7.85 (m, | |
| | | yl)-8-fluoropyrido[4,3- | 1H), 7.83-7.76 (m, 3H), | |
| | | d]pyrimidin-2- | 7.60 (d, J = 2.0 Hz, 1H), | |
| | | yl)oxy)methyl)pyrrolidin- | 7.48-7.42 (m, 4H), 5.40 | |
| | | 1- | (br t, J = 7.2 Hz, 1H), 4.97- | |
| | | yl)propoxy)propanamido)- | 4.90 (m, 2H), 4.85 (br t, J = | |
| | | 3,3- | 8.0 Hz, 1H), 4.77-4.68 | |
| | | dimethylbutanoyl)-4- | (m, 3H), 4.54-4.47 (m, | |
| | | hydroxy-N-((S)-1- | 1H), 4.35 (d, J = 3.2 Hz, | |
| | | (2′,3′,6′-trimethyl-[1,1′- | 1H), 4.12-3.86 (m, 15H), | |
| | | biphenyl]-4- | 3.49-3.44 (m, 1H), 3.34- | |
| | | yl)ethyl)pyrrolidine-2- | 3.20 (m, 2H), 3.11-3.07 | |
| | | carboxamide | (m, 1H), 2.72-2.67 (m, | |
| | | | 1H), 2.64 (s, 3H), 2.60- | |
| | | | 2.56 (m, 1H), 2.45-2.39 | |
| | | | (m, 1H), 2.31 (s, 3H), 2.26 | |
| | | | (s, 3H), 2.16-2.07 (m, | |
| | | | 9H), 1.82 (br d, J = 6.8 Hz, | |
| | | | 3H), 1.33 (br d, J = 2.4 Hz, | |
| | | | 9H | |
| 57 | Ex. 14 | (2S,4R)-1-((2S)-2-(3-(3- | ¹H NMR (400 MHz, | 1181.2 |
| | | ((2S)-2-(((4-(3,8- | DMSO-d₆) δ: 10.14 (d, J = | |
| | | diazabicyclo[3.2.1]octan- | 1.6 Hz, 1H), 9.03 (s, 1H), | |
| | | 3-yl)-7-(8-ethynyl-7- | 8.33 (d, J = 7.6 Hz, 1H), | |
| | | fluoro-3- | 8.03-7.90 (m, 1H), 7.83- | |
| | | hydroxynaphthalen-1- | 7.74 (m, 1H), 7.73-7.67 | |
| | | yl)-8-fluoropyrido[4,3- | (m, 1H), 7.64-7.58 (m, | |
| | | d]pyrimidin-2- | 1H), 7.47-7.36 (m, 4H), | 1131.7 |
| | | yl)oxy)methyl)pyrrolidin- | 7.25-7.16 (m, 3H), 5.14- | |
| | | 1- | 4.91 (m, 2H), 4.54-4.42 | |
| | | yl)propoxy)propanamido)- | (m, 3H), 4.38-4.34 (m, | |
| | | 3,3- | 1H), 4.30 (s, 1H), 4.12- | |
| | | dimethylbutanoyl)-4- | 4.04 (m, 1H), 3.90 (d, J = | |
| | | hydroxy-N-((S)-1- | 2.4 Hz, 1H), 3.63 (d, J = | |
| | | (2′,3′,6′-trichloro-[1,1′- | 6.4 Hz, 2H), 3.59-3.49 | |
| | | biphenyl]-4- | (m, 6H), 3.38 (d, J = 6.0 | |
| | | yl)ethyl)pyrrolidine-2- | Hz, 2H), 3.05-3.01 (m, | |
| | | carboxamide | 1H), 2.91-2.87 (m, 1H), | |
| | | | 2.82-2.78 (m, 1H), 2.21- | |
| | | | 2.15 (m, 1H), 2.03-1.98 | |
| | | | (m, 1H), 1.94-1.89 (m, | |
| | | | 1H), 1.87-1.82 (m, 1H), | |
| | | | 1.71-1.65 (m, 7H), 1.40 | |
| | | | (d, J = 7.2 Hz, 3H), 1.24 (s, | |
| | | | 3H), 0.95-0.85 (m, 12H) | |
| 58 | Ex. 14 | (2S,4R)-1-((2S)-2-(3-(3- | ¹H NMR (400 MHz, | |
| | | ((2S)-2-(((4-(3,8- | DMSO-d₆) δ: 9.04 (s, 1H), | |
| | | diazabicyclo[3.2.1]octan- | 8.43-8.35 (m, 1H), 8.17 | |
| | | 3-yl)-7-(8-ethynyl-7- | (s, 1H), 8.01-7.93 (m, | |
| | | fluoro-3- | 1H), 7.86-7.77 (m, 1H), | |
| | | hydroxynaphthalen-1- | 7.58-7.34 (m, 8H), 7.32- | |
| | | yl)-8-fluoropyrido[4,3- | 7.26 (m, 1H), 7.17 (d, J = | |
| | | d]pyrimidin-2- | 2.4 Hz, 1H), 4.92 (t, J = 7.2 | |
| | | yl)oxy)methyl)pyrrolidin- | Hz, 1H), 4.53-4.46 (m, | |
| | | 1- | 2H), 4.44-4.30 (m, 3H), | |
| | | yl)propoxy)propanamido)- | 4.26 (d, J = 3.2 Hz, 1H), | |
| | | 3,3- | 4.13-4.03 (m, 1H), 3.92 | |
| | | dimethylbutanoyl)-4- | (d, J = 3.2 Hz, 1H), 3.69- | |
| | | hydroxy-N-((S)-1- | 3.53 (m, 8H), 3.07-3.02 | |
| | | (2′,3′,5′-trifluoro-[1,1′- | (m, 1H), 2.94-2.76 (m, | |
| | | biphenyl]-4- | 3H), 2.25-2.17 (m, 2H), | |
| | | yl)ethyl)pyrrolidine-2- | 2.05-1.95 (m, 2H), 1.94- | |
| | | carboxamide | 1.88 (m, 1H), 1.77 (t, J = | |
| | | | 4.8 Hz, 1H), 1.75-1.61 | |
| | | | (m, 10H), 1.37 (d, J = 6.8 | |
| | | | Hz, 3H), 1.23 (s, 2H), 0.90 | |
| | | | (d, J = 2.8 Hz, 9H) | |
| 59 | Ex. 14 | (2S,4R)-1-((2S)-2-(3-(3- | ¹H NMR (400 MHz, | 1168.3 |
| | | ((2S)-2-(((4-(3,8- | DMSO-d₆) δ: 9.04 (s, 1H), | |
| | | diazabicyclo[3.2.1]octan- | 8.40 (d, J = 1.6, 7.6 Hz, | |
| | | 3-yl)-7-(8-ethynyl-7- | 1H), 8.19 (s, 1H), 7.94 (s, | |
| | | fluoro-3- | 1H), 7.87-7.78 (m, 1H), | |
| | | hydroxynaphthalen-1- | 7.39 (d, J = 2.4 Hz, 7H), | |
| | | yl)-8-fluoropyrido[4,3- | 7.15 (s, 1H), 4.99-4.90 | |
| | | d]pyrimidin-2- | (m, 1H), 4.54-4.47 (m, | |
| | | yl)oxy)methyl)pyrrolidin- | 2H), 4.42 (t, J = 8.0 Hz, | |
| | | 1- | 1H), 4.37-4.26 (m, 3H), | |
| | | yl)propoxy)propanamido)- | 4.13-4.03 (m, 1H), 3.93 | |
| | | 3,3- | (d, J = 2.4 Hz, 1H), 3.67 (s, | |
| | | dimethylbutanoyl)-4- | 1H), 3.62-3.53 (m, 7H), | |
| | | hydroxy-N-((S)-1- | 3.50-3.47 (m, 1H), 3.07- | |
| | | (2′,3′,4′,5′,6′- | 3.02 (m, 1H), 2.89-2.77 | |
| | | pentafluoro-[1,1′- | (m, 3H), 2.26 (d, J = 2.8, | |
| | | biphenyl]-4- | 8.0 Hz, 1H), 2.22-2.14 | |
| | | yl)ethyl)pyrrolidine-2- | (m, 2H), 2.04-1.99 (m, | |
| | | carboxamide | 1H), 1.91 (td, J = 2.4, 8.0 | |
| | | | Hz, 1H), 1.74-1.61 (m, | |
| | | | 11H), 1.39 (d, J = 7.2 Hz, | |
| | | | 3H), 0.90 (d, J = 2.8 Hz, | |
| | | | 10H) | |
| 60 | Ex. 15 | (2S,4R)-1-((2R)-2-(3- | ¹H NMR (400 MHz, | 1081.5 |
| | | ((1-(2-((4-(3,8- | DMSO-d₆) δ: 9.03 (s, 1H), | |
| | | diazabicyclo[3.2.1]octan- | 9.00-8.95 (m, 1H), 8.30- | |
| | | 3-yl)-7-(8-ethynyl-7- | 8.18 (m, 2H), 7.96 (dd, J = | |
| | | fluoro-3- | 6.4, 8.8 Hz, 1H), 7.50- | |
| | | hydroxynaphthalen-1- | 7.33 (m, 5H), 7.32-7.29 | |
| | | yl)-8-fluoropyrido[4,3- | (m, 1H), 7.17 (d, J = 2.4 | |
| | | d]pyrimidin-2- | Hz, 1H), 6.12-6.03 (m, | |
| | | yl)oxy)ethyl)piperidin- | 1H), 4.93-4.84 (m, 1H), | |
| | | 4-yl)methoxy)isoxazol- | 4.53-4.24 (m, 7H), 4.00- | |
| | | 5-yl)-3- | 3.92 (m, 3H), 3.68-3.58 | |
| | | methylbutanoyl)-4- | (m, 5H), 3.02-2.87 (m, | |
| | | hydroxy-N-((S)-1-(4-(4- | 3H), 2.74-2.63 (m, 3H), | |
| | | methylthiazol-5- | 2.45-2.43 (m, 3H), 2.31- | |
| | | yl)phenyl)ethyl)pyrrolidine- | 2.16 (m, 2H), 2.11-1.84 | |
| | | 2-carboxamide | (m, 4H), 1.79 (d, J = 2.8 | |
| | | | Hz, 7H), 1.45 (d, J = 7.2 | |
| | | | Hz, 1H), 1.38-1.30 (m, | |
| | | | 3H), 1.28-1.19 (m, 2H), | |
| | | | 0.95 (d, J = 6.8 Hz, 3H), | |
| | | | 0.86-0.79 (m, 3H) | |
| 61 | Ex. 15 | (2S,4R)-1-((2R)-2-(3- | ¹H NMR (400 MHz, | 1110.6 |
| | | ((1-(2-((4-(3,8- | DMSO-d₆) δ: 10.13 (br s, | |
| | | diazabicyclo[3.2.1]octan- | 1H), 9.04 (s, 1H), 8.41 (d, | |
| | | 3-yl)-7-(8-ethynyl-7- | J = 7.8 Hz, 1H), 8.15 (s, | |
| | | fluoro-3- | 1H), 7.97 (dd, J = 5.6, 9.2 | |
| | | hydroxynaphthalen-1- | Hz, 1H), 7.49-7.43 (m, | |
| | | yl)-8-fluoropyrido[4,3- | 2H), 7.38 (s, 4H), 7.24- | |
| | | d]pyrimidin-2- | 7.15 (m, 4H), 6.09 (s, 1H), | |
| | | yl)oxy)ethyl)-4- | 4.92 (t, J = 7.2 Hz, 1H), | |
| | | methylpiperidin-4- | 4.49 (br d, J = 13.2 Hz, | |
| | | yl)methoxy)isoxazol-5- | 1H), 4.45 (br t, J = 5.6 Hz, | |
| | | yl)-3-methylbutanoyl)- | 2H), 4.38-4.31 (m, 2H), | |
| | | N-((S)-1-(2′,6′-difluoro- | 4.27 (br d, J = 2.4 Hz, 1H), | 1114.4 |
| | | [1,1′-biphenyl]-4- | 3.93 (s, 1H), 3.90 (s, 2H), | |
| | | yl)ethyl)-4- | 3.72-3.66 (m, 2H), 3.66- | |
| | | hydroxypyrrolidine-2- | 3.61 (m, 4H), 3.58 (br s, | |
| | | carboxamide | 1H), 3.44 (br d, J = 11.4 | |
| | | | Hz, 1H), 2.75-2.70 (m, | |
| | | | 2H), 2.07-1.99 (m, 2H), | |
| | | | 1.80 (br dd, J = 4.4, 8.4 Hz, | |
| | | | 1H), 1.70 (br s, 4H), 1.57 | |
| | | | (br t, J = 8.8 Hz, 3H), 1.47- | |
| | | | 1.44 (m, 1H), 1.39 (d, J = | |
| | | | 7.1 Hz, 4H), 1.34 (br d, J = | |
| | | | 3.2 Hz, 1H), 1.00-0.93 | |
| | | | (m, 8H), 0.83 (br d, J = 6.4 | |
| | | | Hz, 1H), 0.79 (d, J = 6.4 | |
| | | | Hz, 3H) | |
| 62 | Ex. 15 | (2S,4R)-1-((2R)-2-(3- | ¹H NMR (400 MHz, | |
| | | ((1-(2-((4-(3,8- | DMSO-d₆) δ: 9.05 (s, 1H), | |
| | | diazabicyclo[3.2.1]octan- | 8.80 (s, 1H), 8.85-8.79 | |
| | | 3-yl)-7-(8-ethynyl-7- | (m, 1H), 8.41 (s, 1H), 8.20 | |
| | | fluoro-3- | (d, J = 2.4 Hz, 1H), 7.98 | |
| | | hydroxynaphthalen-1- | (dd, J = 6.0, 8.8 Hz, 1H), | |
| | | yl)-8-fluoropyrido[4,3- | 7.45 (s, 2H), 7.39 (s, 5H), | |
| | | d]pyrimidin-2- | 7.26-7.16 (m, 3H), 6.16 | |
| | | yl)oxy)ethyl)-4- | (s, 1H), 4.98-4.90 (m, | |
| | | fluoropiperidin-4- | 1H), 4.51-4.42 (m, 3H), | |
| | | yl)methoxy)isoxazol-5- | 4.41-4.31 (m, 2H), 4.29- | |
| | | yl)-3-methylbutanoyl)- | 4.17 (m, 3H), 3.95 (s, 1H), | |
| | | N-((S)-1-(2′,6′-difluoro- | 3.75-3.67 (m, 2H), 3.64- | |
| | | [1,1′-biphenyl]-4- | 3.55 (m, 5H), 3.49-3.41 | |
| | | yl)ethyl)-4- | (m, 5H), 2.36-2.29 (m, | |
| | | hydroxypyrrolidine-2- | 4H), 1.73-1.64 (m, 5H), | |
| | | carboxamide | 1.40 (d, J = 7.2 Hz, 3H), | |
| | | | 1.06 (m, 1H), 1.01-0.91 | |
| | | | (m, 4H), 0.85-0.78 (m, | |
| | | | 4H) | |
| 63 | Ex. 15 | (2S,4R)-1-((2R)-2-(3- | ¹H NMR (400 MHz, | 1114.2 |
| | | ((1-(2-((4-(3,8- | DMSO-d₆) δ: 10.23 (s, | |
| | | diazabicyclo[3.2.1]octan- | 1H), 9.12 (s, 1H), 8.44 (d, | |
| | | 3-yl)-7-(8-ethynyl-7- | J = 7.6 Hz, 1H), 8.02-7.95 | |
| | | fluoro-3- | (m, 1H), 7.51-7.44 (m, | |
| | | hydroxynaphthalen-1- | 1H), 7.41-7.37 (m, 5H), | |
| | | yl)-8-fluoropyrido[4,3- | 7.33 (t, J = 8.8 Hz, 2H), | |
| | | d]pyrimidin-2- | 7.18 (d, J = 2.4 Hz, 1H), | |
| | | yl)oxy)ethyl)piperidin- | 6.08 (s, 1H), 5.04 (s, 1H), | |
| | | 4-yl)methoxy)isoxazol- | 4.96-4.88 (m, 1H), 4.71- | |
| | | 5-yl)-3- | 4.59 (m, 2H), 4.59-4.48 | |
| | | methylbutanoyl)-4- | (m, 3H), 4.36 (t, J = 8.0 | |
| | | hydroxy-N-((S)-1- | Hz, 1H), 4.31-4.26 (m, | 1128.5 |
| | | (2′,4′,6′-trifluoro-[1,1′- | 1H), 4.17 (br s, 2H), 4.01 | |
| | | biphenyl]-4- | (br d, J = 4.8 Hz, 2H), 3.95 | |
| | | yl)ethyl)pyrrolidine-2- | (s, 1H), 3.83 (br d, J = 13.6 | |
| | | carboxamide | Hz, 2H), 3.70 (br dd, J = | |
| | | | 4.0, 10.0 Hz, 1H), 3.67- | |
| | | | 3.62 (m, 1H), 3.48-3.38 | |
| | | | (m, 5H),2.27-2.18 (m, | |
| | | | 2H), 2.09-1.98 (m, 2H), | |
| | | | 1.98-1.90 (m, 5H), 1.78 | |
| | | | (ddd, J = 4.8, 7.6, 12.8 Hz, | |
| | | | 4H), 1.46 (br d, J = 7.2 Hz, | |
| | | | 1H), 1.39 (br d, J = 7.2 Hz, | |
| | | | 3H), 0.97-0.94 (m, 3H), | |
| | | | 0.79 (d, J = 6.8 Hz, 3H) | |
| 64 | Ex. 15 | (2S,4R)-1-((2R)-2-(3- | ¹H NMR (400 MHz, | |
| | | ((1-(2-((4-(3,8- | DMSO-d₆) δ: 10.57-9.84 | |
| | | diazabicyclo[3.2.1]octan- | (m, 1H), 9.08 (s, 1H), 8.42 | |
| | | 3-yl)-7-(8-ethynyl-7- | (d, J = 7.6 Hz, 1H), 8.16 (s, | |
| | | fluoro-3- | 1H), 7.99 (dd, J = 6.0, 9.2 | |
| | | hydroxynaphthalen-1- | Hz, 1H), 7.47 (t, J = 9.2 | |
| | | yl)-8-fluoropyrido[4,3- | Hz, 1H), 7.41-7.37 (m, | |
| | | d]pyrimidin-2- | 5H), 7.32 (t, J = 8.8 Hz, | |
| | | yl)oxy)ethyl)-4- | 2H), 7.18 (d, J = 2.4 Hz, | |
| | | methylpiperidin-4- | 1H), 6.10 (s, 1H), 4.93 (m, | |
| | | yl)methoxy)isoxazol-5- | J = 7.2 Hz, 1H), 4.58 (br d, | |
| | | yl)-3-methylbutanoyl)- | J = 11.6 Hz, 1H), 4.48 (br | |
| | | 4-hydroxy-N-((S)-1- | s, 2H), 4.44-4.35 (m, 2H), | |
| | | (2′,4′,6′-trifluoro-[1,1′- | 4.29 (br s, 1H), 3.94 (s, | |
| | | biphenyl]-4- | 1H), 3.93-3.86 (m, 4H), | |
| | | yl)ethyl)pyrrolidine-2- | 3.78-3.68 (m, 4H), 3.65 | |
| | | carboxamide | (br d, J = 10.0 Hz, 2H), | |
| | | | 3.60-3.54 (m, 1H), 2.83- | |
| | | | 2.74 (m, 2H), 2.66-2.61 | |
| | | | (m, 1H), 2.29-2.21 (m, | |
| | | | 1H), 2.08-2.01 (m, 1H), | |
| | | | 1.86-1.77 (m, 5H), 1.60 | |
| | | | (br t, J = 9.6 Hz, 2H), 1.49- | |
| | | | 1.45 (m, 1H), 1.39 (br d, | |
| | | | J = 7.2 Hz, 5H), 1.03-0.93 | |
| | | | (m, 7H), 0.86-0.82 (m, | |
| | | | 1H), 0.82-0.77 (m, 3H) | |
| 65 | Ex. 15 | (2S,4R)-1-((2R)-2-(3- | ¹H NMR (400 MHz, | 1132.4 |
| | | ((1-(2-((4-(3,8- | DMSO-d₆) δ: 9.04 (s, 1H), | |
| | | diazabicyclo[3.2.1]octan- | 8.33 (d, J = 7.6 Hz, 1H), | |
| | | 3-yl)-7-(8-ethynyl-7- | 8.03-7.91 (m, 1H), 7.48- | |
| | | fluoro-3- | 7.37 (m, 3H), 7.34-7.28 | |
| | | hydroxynaphthalen-1- | (m, 5H), 7.17 (d, J = 2.4 | |
| | | yl)-8-fluoropyrido[4,3- | Hz, 1H), 6.22-6.11 (m, | |
| | | d]pyrimidin-2- | 1H), 5.05-4.88 (m, 1H), | |
| | | yl)oxy)ethyl)-4- | 4.53-4.39 (m, 4H), 4.34- | |
| | | fluoropiperidin-4- | 4.17 (m, 4H), 3.94 (d, J = | |
| | | yl)methoxy)isoxazol-5- | 0.8 Hz, 1H), 3.79-3.73 | |
| | | yl)-3-methylbutanoyl)- | (m, 1H), 3.67-3.58 (m, | |
| | | 4-hydroxy-N-((S)-1- | 2H), 3.52-3.41 (m, 3H), | |
| | | (2′,4′,6′-trifluoro-[1,1′- | 2.87-2.70 (m, 4H), 2.38- | |
| | | biphenyl]-4- | 2.20 (m, 5H), 2.09-2.01 | |
| | | yl)ethyl)pyrrolidine-2- | (m, 1H), 1.99-1.74 (m, | |
| | | carboxamide | 5H), 1.66 (s, 5H), 1.47 (d, | |
| | | | J = 7.2 Hz, 1H), 1.35 (d, J = | |
| | | | 7.2 Hz, 2H), 0.96 (d, J = | |
| | | | 6.4 Hz, 2H), 0.84-0.74 | |
| | | | (m, 4H) | |
| 66 | Ex. 17 | (2S,4R)-1-((2R)-2-(3- | ¹H NMR (400 MHz, | |
| | | ((1-(2-((7-(2-amino-3- | DMSO-d₆) δ: 8.42 (d, J = | |
| | | cyano-7- | 7.6 Hz, 1H), 8.19 (s, 1H), | 1149.5 |
| | | fluorobenzo[b]thiophen- | 8.10 (s, 1H), 7.85 (s, 1H), | |
| | | 4-yl)-4-(3,8- | 7.50-7.43 (m, 1H), 7.39 | |
| | | diazabicyclo[3.2.1]octan- | (s, 4H), 7.30-7.11 (m, | |
| | | 3-yl)-6-chloro-8- | 5H), 6.11-5.93 (m, 1H), | |
| | | fluoroquinazolin-2- | 4.93 (t, J = 7.2 Hz, 1H), | |
| | | yl)oxy)ethyl)-4- | 4.52-4.17 (m, 8H), 3.90 | |
| | | methylpiperidin-4- | (s, 2H), 3.73-3.68 (m, | |
| | | yl)methoxy)isoxazol-5- | 1H), 3.63 (s, 3H), 3.53 (d, | |
| | | yl)-3-methylbutanoyl)- | J = 12.0 Hz, 2H), 3.46 (s, | |
| | | N-((S)-1-(2′,6′-difluoro- | 2H), 2.73 (s, 3H), 2.25- | |
| | | [1,1′-biphenyl]-4- | 2.20 (m, 1H), 2.06-1.99 | |
| | | yl)ethyl)-4- | (m, 1H), 1.82-1.76 (m, | |
| | | hydroxypyrrolidine-2- | 1H), 1.62 (s, 7H), 1.39 (d, | |
| | | carboxamide | J = 6.8 Hz, 4H), 0.99-0.94 | |
| | | | (m, 6H), 0.88-0.74 (m, | |
| | | | 4H) | |
| 67 | Ex. 17 | (2S,4R)-1-((2S)-2-(3-(7- | ¹H NMR (400 MHz, | 1164.1 |
| | | (2-((7-(2-amino-3- | DMSO-d₆) δ: 8.25 (s, 1H), | |
| | | cyano-7- | 8.20 (dd, J = 1.6, 7.2 Hz, | |
| | | fluorobenzo[b]thiophen- | 1H), 8.10 (s, 1H), 7.84 (s, | |
| | | 4-yl)-4-(3,8- | 1H), 7.49-7.40 (m, 1H), | |
| | | diazabicyclo[3.2.1]octan- | 7.36-7.22 (m, 6H), 7.18- | |
| | | 3-yl)-6-chloro-8- | 7.09 (m, 1H), 5.86 (s, 1H), | |
| | | fluoroquinazolin-2- | 5.13-4.83 (m, 1H), 4.53- | |
| | | yl)oxy)ethyl)-2,7- | 4.36 (m, 3H), 4.34-4.21 | |
| | | diazaspiro[3.5]nonan-2- | (m, 3H), 3.69 (d, J = 8.8 | |
| | | yl)isoxazol-5-yl)-3- | Hz, 1H), 3.61-3.48 (m, | |
| | | methylbutanoyl)-4- | 10H), 3.45-3.41 (m, 1H), | |
| | | hydroxy-N-((S)-1- | 2.71-2.61 (m, 2H), 2.43- | |
| | | (2′,4′,6′-trifluoro-[1,1′- | 2.32 (m, 4H), 2.29-2.12 | |
| | | biphenyl]-4- | (m, 2H), 2.08-2.00 (m, | |
| | | yl)ethyl)pyrrolidine-2- | 1H), 1.80-1.62 (m, 1H), | |
| | | carboxamide | 1.75-1.55 (m, 8H), 1.46 | |
| | | | (d, J = 7.2 Hz, 1H), 1.36 | |
| | | | (d, J = 7.2 Hz, 2H), 0.95 | |
| | | | (d, J = 6.4 Hz, 2H), 0.87- | |
| | | | 0.71 (m, 4H) | |
| 68 | Ex. 17 | (2S,4R)-1-((2R)-2-(3-(7- | ¹H NMR (400 MHz, | 1164.1 |
| | | (2-((7-(2-amino-3- | DMSO-d₆) δ: 8.82-8.39 | |
| | | cyano-7- | (m, 1H), 8.25 (s, 1H), 8.11 | |
| | | fluorobenzo[b]thiophen- | (s, 1H), 7.84 (s, 1H), 7.45- | |
| | | 4-yl)-4-(3,8- | 7.23 (m, 7H), 7.20-7.07 | |
| | | diazabicyclo[3.2.1]octan- | (m, 1H), 5.87-5.71 (m, | |
| | | 3-yl)-6-chloro-8- | 1H), 4.93 (m, J = 7.2 Hz, | |
| | | fluoroquinazolin-2- | 1H), 4.44-4.19 (m, 6H), | |
| | | yl)oxy)ethyl)-2,7- | 3.70 (dd, J = 4.0, 10.3 Hz, | |
| | | diazaspiro[3.5]nonan-2- | 1H), 3.62-3.49 (m, 10H), | |
| | | yl)isoxazol-5-yl)-3- | 3.41 (d, J = 10.8 Hz, 1H), | |
| | | methylbutanoyl)-4- | 2.71-2.63 (m, 2H), 2.43 | |
| | | hydroxy-N-((S)-1- | (dd, J = 1.6, 15.6 Hz, 4H), | |
| | | (2′,4′,6′-trifluoro-[1,1′- | 2.25-2.14 (m, 2H), 2.07- | |
| | | biphenyl]-4- | 1.94 (m, 1H), 1.82-1.56 | |
| | | yl)ethyl)pyrrolidine-2- | (m, 9H), 1.49-1.36 (m, | |
| | | carboxamide | 3H), 0.98-0.91 (m, 3H), | |
| | | | 0.84-0.76 (m, 3H) | |
| 69 | Ex. 17 | (2S,4R)-1-((2R)-2-(3- | ¹H NMR (400 MHz, | 1171.5 |
| | | ((1-(2-((7-(2-amino-3- | DMSO-d₆) δ: 8.86-8.40 | |
| | | cyano-7- | (m, 1H), 8.22 (s, 1H), 8.11 | |
| | | fluorobenzo[b] thiophen- | (s, 1H), 7.85 (s, 1H), 7.45- | |
| | | 4-yl)-4-(3,8- | 7.21 (m, 8H), 7.19-7.11 | |
| | | diazabicyclo[3.2.1]octan- | (m, 1H), 6.18-5.98 (m, | |
| | | 3-yl)-6-chloro-8- | 1H), 4.92 (t, J = 7.2 Hz, | |
| | | fluoroquinazolin-2- | 1H), 4.45-4.30 (m, 4H), | |
| | | yl)oxy)ethyl)-4- | 4.28-4.18 (m, 4H), 3.72 | |
| | | fluoropiperidin-4- | (d, J = 4.8 Hz, 1H), 3.69- | |
| | | yl)methoxy)isoxazol-5- | 3.64 (m, 3H), 3.60 (s, 3H), | |
| | | yl)-3-methylbutanoyl)- | 2.82-2.73 (m, 5H), 2.34- | |
| | | 4-hydroxy-N-((S)-1- | 2.20 (m, 4H), 1.87-1.75 | |
| | | (2′,4′,6′-trifluoro-[1,1′- | (m, 4H), 1.68-1.59 (m, | |
| | | biphenyl]-4- | 4H), 1.39 (d, J = 6.8 Hz, | |
| | | yl)ethyl)pyrrolidine-2- | 4H), 0.99-0.93 (m, 3H), | |
| | | carboxamide | 0.84-0.76 (m, 3H) | |
| 70 | Ex. 17 | (2S,4R)-1-((2R)-2-(3-(7- | ¹H NMR (400 MHz, | 1147.6 |
| | | (2-((7-(2-amino-3- | DMSO-d₆) δ: 8.39 (d, J = | |
| | | cyano-7- | 7.2 Hz, 1H), 8.14 (s, 1H), | |
| | | fluorobenzo[b]thiophen- | 8.11 (s, 2H), 7.90 (s, 1H), | |
| | | 4-yl)-4-(3,8- | 7.46 (br dd, J = 7.6, 15.6 | |
| | | diazabicyclo[3.2.1]octan- | Hz, 2H), 7.39 (s, 4H), 7.27- | |
| | | 3-yl)-6-chloro-8- | 7.22 (m, 2H), 7.22-7.14 | |
| | | fluoroquinazolin-2- | (m, 2H), 5.83 (s, 1H), 5.09 | |
| | | yl)oxy)ethyl)-2,7- | (br s, 1H), 4.93 (s, 2H), | |
| | | diazaspiro [3.5]nonan-2- | 4.51-4.41 (m, 4H), 4.39- | |
| | | yl)isoxazol-5-yl)-3- | 4.27 (m, 4H), 4.04-3.98 | |
| | | methylbutanoyl)-N-((S)- | (m, 2H), 3.82-3.74 (m, | |
| | | 1-(2′,6′-difluoro-[1,1′- | 2H), 3.74-3.67 (m, 2H), | |
| | | biphenyl]-4-yl)ethyl)-4- | 3.66-3.60 (m, 2H), 3.45- | |
| | | hydroxypyrrolidine-2- | 3.36 (m, 5H), 2.76-2.70 | |
| | | carboxamide | (m, 2H), 1.88-1.78 (m, | |
| | | | 5H), 1.74 (br d, J = 4.8 Hz, | |
| | | | 5H), 1.39 (d, J = 7.2 Hz, | |
| | | | 3H), 0.97-0.93 (m, 3H), | |
| | | | 0.78 (d, J = 6.4 Hz, 3H) | |
| 71 | Ex. 17 | (2S,4R)-1-((2R)-2-(3- | ¹H NMR (400 MHz, | 1153.4 |
| | | ((1-(2-((7-(2-amino-3- | DMSO-d₆) δ: 8.84-8.38 | |
| | | cyano-7- | (m, 1H), 8.17 (s, 1H), 8.10 | |
| | | fluorobenzo[b]thiophen- | (s, 1H), 7.90-7.83 (m, | |
| | | 4-yl)-4-(3,8- | 1H), 7.49-7.21 (m, 8H), | |
| | | diazabicyclo[3.2.1]octan- | 7.19-7.11 (m, 1H), 6.08- | |
| | | 3-yl)-6-chloro-8- | 5.90 (m, 1H), 4.98-4.87 | |
| | | fluoroquinazolin-2- | (m, 1H), 4.49-4.23 (m, | |
| | | yl)oxy)ethyl)piperidin- | 7H), 3.98 (d, J = 5.6 Hz, | |
| | | 4-yl)methoxy)isoxazol- | 2H), 3.72-3.60 (m, 6H), | |
| | | 5-yl)-3- | 3.54 (d, J = 12.0 Hz, 1H), | |
| | | methylbutanoyl)-4- | 3.45-3.42 (m, 2H), 2.97 | |
| | | hydroxy-N-((S)-1- | (d, J = 9.6 Hz, 2H), 2.71 (s, | |
| | | (2′,4′,6′-trifluoro-[1,1′- | 2H), 2.28-2.22 (m, 1H), | |
| | | biphenyl]-4- | 2.10-1.98 (m, 4H), 1.72- | |
| | | yl)ethyl)pyrrolidine-2- | 1.64 (m, 6H), 1.39 (d, J = | |
| | | carboxamide | 7.2 Hz, 3H), 1.27 (d, J = | |
| | | | 11.6 Hz, 1H), 0.98-0.92 | |
| | | | (m, 3H), 0.84-0.75 (m, | |
| | | | 3H) | |
| 72 | Ex. 17 | (2S,4R)-1-((2R)-2-(3- | ¹H NMR (400 MHz, | 1167.6 |
| | | ((1-(2-((7-(2-amino-3- | DMSO-d₆) δ: 8.83-8.39 | |
| | | cyano-7- | (m, 1H), 8.14 (s, 1H), 8.10 | |
| | | fluorobenzo[b]thiophen- | (s, 1H), 7.87 (s, 1H), 7.47- | |
| | | 4-yl)-4-(3,8- | 7.34 (m, 5H), 7.33-7.28 | |
| | | diazabicyclo[3.2.1]octan- | (m, 2H), 7.24 (dd, J = 5.6, | |
| | | 3-yl)-6-chloro-8- | 8.4 Hz, 1H), 7.18-7.12 | |
| | | fluoroquinazolin-2- | (m, 1H), 6.12-5.91 (m, | |
| | | yl)oxy)ethyl)-4- | 1H), 4.98-4.84 (m, 1H), | |
| | | methylpiperidin-4- | 4.52-4.24 (m, 7H), 3.92- | |
| | | yl)methoxy)isoxazol-5- | 3.83 (m, 4H), 3.75-3.56 | |
| | | yl)-3-methylbutanoyl)- | (m, 6H), 3.47-3.43 (m, | |
| | | 4-hydroxy-N-((S)-1- | 2H), 2.78 (d, J = 3.6 Hz, | |
| | | (2′,4′,6′-trifluoro-[1,1′- | 2H), 2.27-2.17 (m, 2H), | |
| | | biphenyl]-4- | 2.07-1.98 (m, 1H), 1.82- | |
| | | yl)ethyl)pyrrolidine-2- | 1.73 (m, 5H), 1.58 (t, J = | |
| | | carboxamide | 9.2 Hz, 2H), 1.45 (d, J = | |
| | | | 6.4 Hz, 1H), 1.41-1.35 | |
| | | | (m, 4H), 1.00-0.92 (m, | |
| | | | 6H), 0.84-0.77 (m, 3H) | |
| 73 | Ex. 17 | (2S,4R)-1-((2R)-2-(3- | ¹H NMR (400 MHz, | 1153.5 |
| | | ((1-(2-((7-(2-amino-3- | DMSO-d₆) δ: 8.44-8.40 | |
| | | cyano-7- | (m, 1H), 8.14 (br d, J = 6.0 | |
| | | fluorobenzo[b]thiophen- | Hz, 2H), 7.95-7.90 (m, | |
| | | 4-yl)-4-(3,8- | 1H), 7.52-7.43 (m, 2H), | |
| | | diazabicyclo[3.2.1]octan- | 7.42-7.39 (m, 5H), 7.26- | |
| | | 3-yl)-6-chloro-8- | 7.21 (m, 3H), 6.17 (s, 1H), | |
| | | fluoroquinazolin-2- | 5.11 (d, J = 3.6 Hz, 1H), | |
| | | yl)oxy)ethyl)-4- | 5.00-4.90 (m, 1H), 4.56- | |
| | | fluoropiperidin-4- | 4.50 (m, 2H), 4.41-4.36 | |
| | | yl)methoxy)isoxazol-5- | (m, 2H), 4.30 (br d, J = 2.4 | |
| | | yl)-3-methylbutanoyl)- | Hz, 3H), 4.22-4.16 (m, | |
| | | N-((S)-1-(2′,6′-difluoro- | 2H), 3.94-3.86 (m, 1H), | |
| | | [1,1′-biphenyl]-4- | 3.78-3.65 (m, 4H), 3.50- | |
| | | yl)ethyl)-4- | 3.42 (m, 2H), 2.69-2.66 | |
| | | hydroxypyrrolidine-2- | (m, 2H), 2.37-2.31 (m, | |
| | | carboxamide | 4H), 1.98-1.88 (m, 7H), | |
| | | | 1.85-1.72 (m, 4H), 1.40 | |
| | | | (d, J = 7.2 Hz, 3H), 1.00- | |
| | | | 0.97 (m, 3H), 0.86-0.80 | |
| | | | (m, 3H) | |
| 74 | Ex. 18 | (2S,4R)-1-((2R)-2-(3- | ¹H NMR (400 MHz, | 1183.5 |
| | | ((1-(2-((7-(2-amino-3- | DMSO-d₆) δ: 8.86-8.37 | |
| | | cyano-7- | (m, 1H), 8.16 (s, 1H), 8.07 | |
| | | fluorobenzo[b]thiophen- | (s, 3H), 7.51-7.36 (m, | |
| | | 4-yl)-4-(3,8- | 5H), 7.29-7.17 (m, 3H), | |
| | | diazabicyclo[3.2.1]octan- | 7.13 (t, J = 8.8 Hz, 1H), | |
| | | 3-yl)-8-fluoro-6- | 6.13-5.92 (m, 1H), 4.98- | |
| | | (trifluoromethyl)quinazolin- | 4.88 (m, 1H), 4.46 (t, J = | |
| | | 2-yl)oxy)ethyl)-4- | 5.6 Hz, 2H), 4.41-4.24 | |
| | | methylpiperidin-4- | (m, 4H), 3.90 (s, 2H), 3.76- | |
| | | yl)methoxy)isoxazol-5- | 3.57 (m, 8H), 2.74 (t, J = | |
| | | yl)-3-methylbutanoyl)- | 4.8 Hz, 2H), 2.65-2.58 | |
| | | N-((S)-1-(2′,6′-difluoro- | (m, 2H), 2.39 (t, J = 9.2 | |
| | | [1,1′-biphenyl]-4- | Hz, 2H), 2.28-2.19 (m, | |
| | | yl)ethyl)-4- | 1H), 2.08-1.99 (m, 1H), | |
| | | hydroxypyrrolidine-2- | 1.84-1.76 (m, 1H), 1.73- | |
| | | carboxamide | 1.53 (m, 6H), 1.47-1.38 | |
| | | | (m, 3H), 1.37-1.22 (m, | |
| | | | 2H), 1.02-0.92 (m, 6H), | |
| | | | 0.86-0.75 (m, 3H) | |
| 75 | Ex. 18 | (2S,4R)-1-((2R)-2-(3-(7- | ¹H NMR (400 MHz, | 1198.4 |
| | | (2-((7-(2-amino-3- | DMSO-d₆) δ: 8.84-8.34 | |
| | | cyano-7- | (m, 1H), 8.08 (s, 3H), 7.46- | |
| | | fluorobenzo[b]thiophen- | 7.20 (m, 7H), 7.18-7.07 | |
| | | 4-yl)-4-(3,8- | (m, 1H), 5.87-5.70 (m, | |
| | | diazabicyclo[3.2.1]octan- | 1H), 5.22-4.87 (m, 2H), | |
| | | 3-yl)-8-fluoro-6- | 4.53-4.22 (m, 6H), 3.90 | |
| | | (trifluoromethyl)quinazolin- | (d, J = 7.2 Hz, 2H), 3.83- | |
| | | 2-yl)oxy)ethyl)-2,7- | 3.63 (m, 4H), 3.62-3.44 | |
| | | diazaspiro[3.5]nonan-2- | (m, 8H), 2.71 (s, 2H), 2.26- | |
| | | yl)isoxazol-5-yl)-3- | 2.11 (m, 2H), 2.07-2.00 | |
| | | methylbutanoyl)-4- | (m, 1H), 1.90-1.66 (m, | |
| | | hydroxy-N-((S)-1- | 9H), 1.38 (d, J = 6.8 Hz, | |
| | | (2′,4′,6′-trifluoro-[1,1′- | 3H), 1.01-0.90 (m, 3H), | |
| | | biphenyl]-4- | 0.84-0.71 (m, 3H) | |
| | | yl)ethyl)pyrrolidine-2- | | |
| | | carboxamide | | |
| 76 | Ex. 18 | (2S,4R)-1-((2R)-2-(3- | ¹H NMR (400 MHz, | 1201.4 |
| | | ((1-(2-((7-(2-amino-3- | DMSO-d₆) δ: 8.42 (d, J = | |
| | | cyano-7- | 7.6 Hz, 1H), 8.16 (s, 1H), | |
| | | fluorobenzo[b]thiophen- | 8.08 (s, 3H), 7.42-7.36 | |
| | | 4-yl)-4-(3,8- | (m, 4H), 7.32 (t, J = 8.8 | |
| | | diazabicyclo[3.2.1]octan- | Hz, 2H), 7.28-7.23 (m, | |
| | | 3-yl)-8-fluoro-6- | 1H), 7.14 (t, J = 8.8 Hz, | |
| | | (trifluoromethyl)quinazolin- | 1H), 6.11-5.93 (m, 1H), | |
| | | 2-yl)oxy)ethyl)-4- | 4.93 (t, J = 7.2 Hz, 1H), | |
| | | methylpiperidin-4- | 4.51-4.43 (m, 2H), 4.42- | |
| | | yl)methoxy)isoxazol-5- | 4.32 (m, 3H), 4.32-4.24 | |
| | | yl)-3-methylbutanoyl)- | (m, 1H), 3.91 (s, 2H), 3.76- | |
| | | 4-hydroxy-N-((S)-1- | 3.59 (m, 7H), 3.44 (s, | |
| | | (2′,4′,6′-trifluoro-[1,1′- | 3H), 2.80-2.72 (m, 2H), | |
| | | biphenyl]-4- | 2.36-2.17 (m, 2H), 2.10- | |
| | | yl)ethyl)pyrrolidine-2- | 1.99 (m, 1H), 1.79 (s, 1H), | |
| | | carboxamide | 1.73-1.64 (m, 3H), 1.64- | |
| | | | 1.52 (m, 3H), 1.49-1.24 | |
| | | | (m, 6H), 1.01-0.94 (m, | |
| | | | 6H), 0.85-0.78 (m, 3H) | |
| 77 | Ex. 18 | (2S,4R)-1-((2R)-2-(3-(7- | ¹H NMR (400 MHz, | 1180.1 |
| | | (2-((7-(2-amino-3- | DMSO-d₆) δ: 8.41 (d, J = | |
| | | cyano-7- | 7.6 Hz, 1H), 8.21-8.04 | |
| | | fluorobenzo[b]thiophen- | (m, 3H), 7.50-7.41 (m, | |
| | | 4-yl)-4-(3,8- | 1H), 7.41-7.35 (m, 4H), | |
| | | diazabicyclo[3.2.1]octan- | 7.28-7.17 (m, 3H), 7.13 | |
| | | 3-yl)-8-fluoro-6- | (t, J = 8.8 Hz, 1H), 5.83 (s, | |
| | | (trifluoromethyl)quinazolin- | 1H), 4.93 (t, J = 7.2 Hz, | |
| | | 2-yl)oxy)ethyl)-2,7- | 1H), 4.97-4.89 (m, 1H), | |
| | | diazaspiro[3.5]nonan-2- | 4.44 (br t, J = 5.6 Hz, 2H), | |
| | | yl)isoxazol-5-yl)-3- | 4.36 (t, J = 8.0 Hz, 1H), | |
| | | methylbutanoyl)-N-((S)- | 4.29 (br s, 2H), 3.71 (br dd, | |
| | | 1-(2′,6′-difluoro-[1,1′- | J = 4.0, 10.4 Hz, 1H), 3.63 | |
| | | biphenyl]-4-yl)ethyl)-4- | (br s, 1H), 3.61-3.51 (m, | |
| | | hydroxypyrrolidine-2- | 8H), 3.47-3.40 (m, 3H), | |
| | | carboxamide | 2.70-2.64 (m, 2H), 2.42- | |
| | | | 2.32 (m, 4H), 2.24-2.10 | |
| | | | (m, 2H), 2.06-1.98 (m, | |
| | | | 1H), 1.72 (br s, 4H), 1.66- | |
| | | | 1.49 (m, 4H), 1.48-1.37 | |
| | | | (m, 3H), 0.99-0.92 (m, | |
| | | | 3H), 0.85-0.75 (m, 3H) | |
| 78 | Ex. 18 | (2S,4R)-1-((2R)-2-(3- | ¹H NMR (400 MHz, | 1187.4 |
| | | ((1-(2-((7-(2-amino-3- | DMSO-d₆)δ: 8.29 (d, J = | |
| | | cyano-7- | 7.6 Hz, 1H), 8.15 (s, 1H), | |
| | | fluorobenzo[b]thiophen- | 8.07 (s, 2H), 7.48-7.36 | |
| | | 4-yl)-4-(3,8- | (m, 1H), 7.35-7.22 (m, | |
| | | diazabicyclo[3.2.1]octan- | 6H), 7.18-7.09 (m, 1H), | |
| | | 3-yl)-8-fluoro-6- | 6.14-6.03 (m, 1H), 5.22- | |
| | | (trifluoromethyl)quinazolin- | 4.23 (m, 8H), 4.03-3.92 | |
| | | 2- | (m, 2H), 3.77-3.50 (m, | |
| | | yl)oxy)ethyl)piperidin- | 8H), 3.02-2.91 (m, 2H), | |
| | | 4-yl)methoxy)isoxazol- | 2.77-2.67 (m, 2H), 2.27- | |
| | | 5-yl)-3- | 2.21 (m, 1H), 2.09-1.97 | |
| | | methylbutanoyl)-4- | (m, 3H), 1.80-1.61 (m, | |
| | | hydroxy-N-((S)-1- | 8H), 1.48-1.34 (m, 3H), | |
| | | (2′,4′,6′-trifluoro-[1,1′- | 1.29-1.17 (m, 2H), 0.98- | |
| | | biphenyl]-4- | 0.74 (m, 6H) | |
| | | yl)ethyl)pyrrolidine-2- | | |
| | | carboxamide | | |
| 79 | Ex. 20 | (2S,4R)-1-((2S)-2-(3-(3- | ¹H NMR (400 MHz, | 1188.1 |
| | | ((2S)-2-((((7R)-7-(2- | DMSO-d₆) δ: 8.40 (d, J = | |
| | | amino-3-cyano-7- | 7.6 Hz, 1H), 8.19 (s, 1H), | |
| | | fluorobenzo[b]thiophen- | 8.09 (d, J = 7.2 Hz, 3H), | |
| | | 4-yl)-4-(3,8- | 7.84 (d, J = 9.2 Hz, 1H), | |
| | | diazabicyclo[3.2.1]octan- | 7.53-7.37 (m, 5H), 7.28- | |
| | | 3-yl)-8-fluoro-6- | 7.18 (m, 3H), 7.14 (t, J = | |
| | | (trifluoromethyl)quinazolin- | 8.8 Hz, 1H), 4.98-4.89 | |
| | | 2- | (m, 1H), 4.52 (d, J = 9.2 | |
| | | yl)oxy)methyl)pyrrolidin- | Hz, 1H), 4.48-4.31 (m, | |
| | | 1- | 5H), 4.28 (s, 1H), 4.11 (dd, | |
| | | yl)propoxy)propanamido)- | J = 7.6, 10.4 Hz, 1H), 3.76 | |
| | | 3,3- | (s, 3H), 3.63-3.55 (m, | |
| | | dimethylbutanoyl)-N- | 3H), 3.41-3.35 (m, 2H), | |
| | | ((S)-1-(2′,6′-difluoro- | 3.10-3.02 (m, 1H), 2.96- | |
| | | [1,1′-biphenyl]-4- | 2.80 (m, 2H), 2.42-2.11 | |
| | | yl)ethyl)-4- | (m, 5H), 2.07-1.98 (m, | |
| | | hydroxypyrrolidine-2- | 1H), 1.97-1.87 (m, 1H), | |
| | | carboxamide | 1.85-1.62 (m, 11H), 1.39 | |
| | | | (d, J = 7.2 Hz, 3H), 0.90 (s, | |
| | | | 9H) | |
| 80 | Ex. 21 | (2S,4R)-1-((2S)-2-(3-(3- | ¹H NMR (400 MHz, | 577.9 |
| | | ((2S)-2-(((7-(2-amino-3- | DMSO-d₆) δ: 8.98 (s, 1H), | [M/2 + 1] |
| | | cyano-7- | 8.38-8.33 (m, 1H), 8.27- | |
| | | fluorobenzo[b]thiophen- | 8.22 (m, 2H), 8.20 (s, 1H), | |
| | | 4-yl)-4-(3,8- | 8.04-7.93 (m, 2H), 7.84- | |
| | | diazabicyclo[3.2.1]octan- | 7.77 (m, 1H), 7.46-7.36 | |
| | | 3-yl)-6- | (m, 5H), 7.24-7.18 (m, | |
| | | (trifluoromethyl)quinazolin- | 1H), 7.10-7.04 (m, 1H), | |
| | | 2- | 4.94-4.88 (m, 1H), 4.54- | |
| | | yl)oxy)methyl)pyrrolidin- | 4.49 (m, 1H), 4.42-4.32 | |
| | | 1- | (m, 3H), 4.29-4.13 (m, | |
| | | yl)propoxy)propanamido)- | 3H), 4.09-3.96 (m, 2H), | |
| | | 3,3- | 3.67-3.53 (m, 10H), 3.06- | |
| | | dimethylbutanoyl)-4- | 2.98 (m, 2H), 2.92-2.75 | |
| | | hydroxy-N-((S)-1-(4-(4- | (m, 3H), 2.45 (s, 3H), 2.27- | |
| | | methylthiazol-5- | 2.13 (m, 2H), 2.03-1.96 | |
| | | yl)phenyl)ethyl)pyrrolidine- | (m, 1H), 1.95-1.87 (m, | |
| | | 2-carboxamide | 1H), 1.82-1.75 (m, 1H), | |
| | | | 1.73-1.58 (m, 8H), 1.55- | |
| | | | 1.48 (m, 1H), 1.36 (d, J = | |
| | | | 6.8 Hz, 3H), 0.89 (s, 9H) | |
| 81 | Ex. 21 | (2S,4R)-1-((2S)-2-(3-(3- | ¹H NMR (400 MHz, | 1187.4 |
| | | ((2S)-2-(((7-(2-amino-3- | DMSO-d₆) | |
| | | cyano-7- | δ: 8.40-8.36 (m, 1H), 8.25- | |
| | | fluorobenzo[b]thiophen- | 8.22 (m, 1H), 8.20 (s, | |
| | | 4-yl)-4-(3,8- | 1H), 7.97 (s, 1H), 7.81- | |
| | | diazabicyclo[3.2.1]octan- | 7.79 (m, 1H), 7.46-7.40 | |
| | | 3-yl)-6- | (m, 6H), 7.38-7.34 (m, | |
| | | (trifluoromethyl)quinazolin- | 2H), 7.33-7.30 (m, 1H), | |
| | | 2- | 7.12-7.07 (m, 1H), 4.93 | |
| | | yl)oxy)methyl)pyrrolidin- | (d, J = 6.4 Hz, 1H), 4.52- | |
| | | 1- | 4.49 (m, 1H), 4.43-4.41 | |
| | | yl)propoxy)propanamido)- | (m, 2H), 4.39-4.36 (m, | |
| | | 3,3- | 2H), 4.27-4.22 (m, 2H), | |
| | | dimethylbutanoyl)-4- | 4.10-4.02 (m, 2H), 3.33- | |
| | | hydroxy-N-((S)-1- | 3.25 (m, 8H), 3.05-3.03 | |
| | | (2′,4′,6′-trifluoro-[1,1′- | (m, 2H), 2.79-2.76 (m, | |
| | | biphenyl]-4- | 1H), 2.74-2.70 (m, 1H), | |
| | | yl)ethyl)pyrrolidine-2- | 2.48-2.45 (m, 1H), 2.36- | |
| | | carboxamide | 2.27 (m, 3H), 2.19-2.17 | |
| | | | (m, 1H), 1.82-1.79 (m, | |
| | | | 1H), 1.77-1.73 (m, 1H), | |
| | | | 1.67-1.61 (m, 9H), 1.55- | |
| | | | 1.48 (m, 1H), 1.37 (d, J = | |
| | | | 6.8 Hz, 3H), 0.89 (s, 9H) | |
| 82 | Ex. 24 | (2S,4R)-1-((2S)-2-(5- | ¹H NMR (400 MHz, | 1045.4 |
| | | ((2S)-2-(((4-(3,8- | DMSO-d₆) δ: 9.09 (s, 1H), | |
| | | diazabicyclo[3.2.1]octan- | 8.98 (s, 1H), 8.37 (d, J = | |
| | | 3-yl)-8-fluoro-7-(8- | 8.0Hz, 1H), 8.28 (s, 2H), | |
| | | fluoro-3- | 7.77 (d, J = 9.2 Hz, 1H), | |
| | | hydroxynaphthalen-1- | 7.66 (d, J = 8.4 Hz, 1H), | |
| | | yl)pyrido[4,3- | 7.48-7.34 (m, 6H), 7.16 | |
| | | d]pyrimidin-2- | (d, J = 2.4 Hz, 1H), 7.00 | |
| | | yl)oxy)methyl)pyrrolidin- | (d, J = 8.0, 13.2 Hz, 1H), | |
| | | 1-yl)pentanamido)- | 4.95-4.88 (m, 1H), 4.53- | |
| | | 3,3-dimethylbutanoyl)- | 4.36 (m, 6H), 4.27 (s, 1H), | |
| | | 4-hydroxy-N-((S)-1-(4- | 4.10 (m, J = 7.6 Hz, 1H), | |
| | | (4-methylthiazol-5- | 3.59 (b s, 10H), 3.07-2.74 | |
| | | yl)phenyl)ethyl)pyrrolidine- | (m, 2H), 2.45 (s, 3H), 2.31- | |
| | | 2-carboxamide | 2.08 (m, 3H), 2.07-1.97 | |
| | | | (m, 1H), 1.96-1.84 (m, | |
| | | | 1H), 1.79 (ddd, J = 4.8, | |
| | | | 8.4, 12.8 Hz, 1H), 1.73- | |
| | | | 1.57 (m, 7H), 1.48-1.33 | |
| | | | (m, 6H), 0.90 (s, 9H) | |
| 83 | Ex. 24 | (2S,4R)-1-((2S)-2-(5- | ¹H NMR (400 MHz, | 1024.6 |
| | | ((2S)-2-(((4-(3,8- | DMSO-d₆) δ: 9.08 (s, 1H), | |
| | | diazabicyclo[3.2.1]octan- | 8.35 (d, J = 7.6 Hz, 1H), | |
| | | 3-yl)-8-fluoro-7-(8- | 8.30-8.23 (m, 2H), 7.76 | |
| | | fluoro-3- | (d, J = 8.8 Hz, 1H), 7.68- | |
| | | hydroxynaphthalen-1- | 7.54 (m, 6H), 7.49-7.38 | |
| | | yl)pyrido[4,3- | (m, 4H), 7.38-7.32 (m, | |
| | | d]pyrimidin-2- | 4H), 7.15 (s, 1H), 6.99 (d, | |
| | | yl)oxy)methyl)pyrrolidin- | J = 7.2, 13.2 Hz, 1H), 4.90 | |
| | | 1-yl)pentanamido)- | (s, 1H), 4.50-4.40 (m, | |
| | | 3,3-dimethylbutanoyl)- | 6H), 4.26 (s, 2H), 4.12- | |
| | | N-((S)-1-([1,1′- | 4.08 (m, 2H), 3.58 (s, 3H), | |
| | | biphenyl]-4-yl)ethyl)-4- | 3.03 (d, J = 4.0 Hz, 1H), | |
| | | hydroxypyrrolidine-2- | 2.82-2.76 (m, 3H), 2.25 | |
| | | carboxamide | (d, J = 5.6 Hz, 1H), 2.19- | |
| | | | 2.10 (m, 2H), 1.99 (s, 1H), | |
| | | | 1.89 (d, J = 9.2 Hz, 1H), | |
| | | | 1.81-1.76 (m, 1H), 1.72- | |
| | | | 1.61 (m, 7H), 1.54 (dd, J = | |
| | | | 4.4, 6.0 Hz, 1H), 1.44- | |
| | | | 1.33 (m, 6H), 0.90 (s, 9H) | |
| 84 | Ex. 24 | (2S,4R)-1-((2S)-2-(5- | ¹H NMR (400 MHz, | 1108.4 |
| | | ((2S)-2-(((7-(2-amino-3- | DMSO-d₆) δ: 8.98 (s, 1H), | |
| | | cyano-7- | 8.37 (d, J = 7.6 Hz, 1H), | |
| | | fluorobenzo[b]thiophen- | 8.27 (s, 2H), 8.10 (s, 2H), | |
| | | 4-yl)-4-(3,8- | 7.87-7.80 (m, 1H), 7.76 | |
| | | diazabicyclo[3.2.1]octan- | (d, J = 8.8 Hz, 1H), 7.46- | |
| | | 3-yl)-6-chloro-8- | 7.41 (m, 2H), 7.39-7.35 | |
| | | fluoroquinazolin-2- | (m, 2H), 7.30-7.24 (m, | |
| | | yl)oxy)methyl)pyrrolidin- | 1H), 7.14 (t, J = 8.8 Hz, | |
| | | 1-yl)pentanamido)- | 1H), 4.94-4.88 (m, 1H), | |
| | | 3,3-dimethylbutanoyl)- | 4.50 (d, J = 8.8 Hz, 1H), | |
| | | 4-hydroxy-N-((S)-1-(4- | 4.44-4.34 (m, 2H), 4.34- | |
| | | (4-methylthiazol-5- | 4.21 (m, 4H), 4.07-4.02 | |
| | | yl)phenyl)ethyl)pyrrolid | (m, 1H), 3.59 (s, 2H), 3.52 | |
| | | 2-carboxamideine- | (s, 4H), 3.05-2.99 (m, | |
| | | | 2H), 2.83-2.76 (m, 2H), | |
| | | | 2.45 (s, 3H), 2.30-2.21 | |
| | | | (m, 2H), 2.15-2.08 (m, | |
| | | | 2H), 2.04-1.86 (m, 3H), | |
| | | | 1.80-1.74 (m, 1H), 1.71- | |
| | | | 1.56 (m, 8H), 1.42 (d, J = | |
| | | | 5.2 Hz, 2H), 1.36 (d, J = | |
| | | | 6.8 Hz, 3H), 0.90 (d, J = | |
| | | | 3.6 Hz, 9H) | |
| 85 | Ex. 24 | (2S,4R)-1-((2S)-2-(5- | ¹H NMR (400 MHz, | 1042.5 |
| | | ((2S)-2-(((4-(3,8- | DMSO-d₆) δ: 9.09-9.06 | |
| | | diazabicyclo[3.2.1]octan- | (m, 1H), 8.24 (m, 1H), 8.24 | |
| | | 3-yl)-8-fluoro-7-(8- | (m, 1H), 7.67-7.65 (m, | |
| | | fluoro-3- | 1H), 7.48-7.47 (m, 1H), | |
| | | hydroxynaphthalen-1- | 7.39-7.16 (m, 11H), 7.15- | |
| | | yl)pyrido[4,3- | 7.00 (m, 1H), 4.91 (d, J = | |
| | | d]pyrimidin-2- | 4.4 Hz, 1H), 4.50 (d, J = | |
| | | yl)oxy)methyl)pyrrolidin- | 9.2 Hz, 4H), 4.41 (d, J = | |
| | | 1-yl)pentanamido)- | 8.8 Hz, 4H), 4.26 (s, 2H), | |
| | | 3,3-dimethylbutanoyl)- | 4.15 (d, J = 6.0 Hz, 2H), | |
| | | N-((S)-1-(2′-fluoro- | 3.84 (s, 2H), 3.58 (s, 4H), | |
| | | [1,1′-bipheny]]-4- | 2.89-2.85 (m, 3H), 2.38 | |
| | | yl)ethyl)-4- | (dd, J = 1.6, 4.4 Hz, 1H), | |
| | | hydroxypyrrolidine-2- | 2.24 (d, J = 1.6 Hz, 1H), | |
| | | carboxamide | 2.15-2.08 (m, 2H), 2.02- | |
| | | | 1.95 (m, 2H), 1.78 (s, 5H), | |
| | | | 1.43 (s, 3H), 1.39-1.34 | |
| | | | (m, 3H), 0.90 (s, 9H) | |
| 86 | Ex. 24 | (2S,4R)-1-((2S)-2-(5- | ¹H NMR (400 MHz, | 1042.5 |
| | | ((2S)-2-(((4-(3,8- | DMSO-d₆) δ: 10.36-10.18 | |
| | | diazabicyclo[3.2.1]octan- | (m, 1H), 9.12 (d, J = 3.6 | |
| | | 3-yl)-8-fluoro-7-(8- | Hz, 1H), 8.41-8.30 (m, | |
| | | fluoro-3- | 1H), 8.19-8.10 (m, 1H), | |
| | | hydroxynaphthalen-1- | 7.79-7.73 (m, 1H), 7.71- | |
| | | yl)pyrido[4,3- | 7.61 (m, 4H), 7.54-7.45 | |
| | | d]pyrimidin-2- | (m, 4H), 7.40-7.34 (m, | |
| | | yl)oxy)methyl)pyrrolidin- | 3H), 7.21-7.13 (m, 2H), | |
| | | 1-yl)pentanamido)- | 7.04-6.97 (m, 1H), 4.91 | |
| | | 3,3-dimethylbutanoyl)- | (d, J = 4.4 Hz, 1H), 4.50 | |
| | | N-((S)-1-(3′-fluoro- | (d, J = 9.2 Hz, 4H), 4.41 | |
| | | [1,1′-biphenyl]-4- | (d, J = 8.8 Hz, 4H), 4.26 (s, | |
| | | yl)ethyl)-4- | 2H), 4.15 (d, J = 6.0 Hz, | |
| | | hydroxypyrrolidine-2- | 2H), 3.84 (s, 2H), 3.58 (s, | |
| | | carboxamide | 4H), 2.89-2.85 (m, 3H), | |
| | | | 2.38 (dd, J = 1.6, 4.4 Hz, | |
| | | | 1H), 2.24 (d, J = 1.6 Hz, | |
| | | | 1H), 2.15-2.08 (m, 2H), | |
| | | | 2.02-1.95 (m, 2H), 1.78 | |
| | | | (s, 5H), 1.43 (s, 3H), 1.39- | |
| | | | 1.34 (m, 3H), 0.90 (s, 9H) | |
| 87 | Ex. 24 | (2S,4R)-1-((2S)-2-(5- | ¹H NMR (400 MHz, | 1042.5 |
| | | ((2S)-2-(((4-(3,8- | DMSO-d₆) δ: 10.43-10.14 | |
| | | diazabicyclo[3.2.1]octan- | (m, 1H), 9.09 (s, 1H), 8.36 | |
| | | 3-yl)-8-fluoro-7-(8- | (d, J = 8.0 Hz, 1H), 7.77 | |
| | | fluoro-3- | (d, J = 8.8 Hz, 1H), 7.73- | |
| | | hydroxynaphthalen-1- | 7.63 (m, 3H), 7.58 (d, J = | |
| | | yl)pyrido[4,3- | 8.4 Hz, 2H), 7.46-7.33 | |
| | | d]pyrimidin-2- | (m, 4H), 7.28 (t, J = 8.8 | |
| | | yl)oxy)methyl)pyrrolidin- | Hz, 2H), 7.16 (d, J = 2.5 | |
| | | 1-yl)pentanamido)- | Hz, 1H), 7.01 (dd, J = 7.2, | |
| | | 3,3-dimethylbutanoyl)- | 12.8 Hz, 1H), 4.91 (t, J = | |
| | | N-((S)-1-(4′-fluoro- | 6.8 Hz, 1H), 4.56-4.35 | |
| | | [1,1′-biphenyl]-4- | (m, 6H), 4.27 (s, 1H), 4.11- | |
| | | yl)ethyl)-4- | 4.06 (m, 1H), 3.59-3.57 | |
| | | hydroxypyrrolidine-2- | (m, 5H), 2.82-2.78 (m, | |
| | | carboxamide | 2H), 2.27-2.22 (m, 1H), | |
| | | | 2.19-2.11 (m, 2H), 2.04- | |
| | | | 1.98 (m, 1H), 1.91-1.87 | |
| | | | (m, 1H), 1.75-1.56 (m, | |
| | | | 9H), 1.48-1.33 (m, 7H), | |
| | | | 1.24 (s, 2H), 0.91 (s, 9H) | |
| 88 | Ex. 24 | (2S,4R)-1-((2S)-2-(5- | ¹H NMR (400 MHz, | 1124.4 |
| | | ((2S)-2-(((7-(2-amino-3- | DMSO-d₆) δ: 8.98 (s, 1H), | |
| | | cyano-7- | 8.38 (d, J = 8.0 Hz, 1H), | |
| | | fluorobenzo[b]thiophen- | 8.32 (s, 3H), 8.10 (s, 1H), | |
| | | 4-yl)-4-(3,8- | 8.13-8.07 (m, 1H), 7.83 | |
| | | diazabicyclo[3.2.1]octan- | (s, 1H), 7.78 (br d, J = 8.0 | |
| | | 3-yl)-6-chloro-8- | Hz, 1H), 7.46-7.37 (m, | |
| | | fluoroquinazolin-2- | 4H), 7.29-7.22 (m, 1H), | |
| | | yl)oxy)methyl)pyrrolidin- | 7.18-7.11 (m, 1H), 4.89- | |
| | | 1-yl)pentanamido)- | 4.82 (m, 1H), 4.54-4.42 | |
| | | 3,3-dimethylbutanoyl)- | (m, 2H), 4.40-4.32 (m, | |
| | | 4-hydroxy-N-((R)-2- | 1H), 4.30-4.22 (m, 3H), | |
| | | hydroxy-1-(4-(4- | 4.08-3.99 (m, 1H), 3.63- | |
| | | methylthiazol-5- | 3.56 (m, 7H), 3.56-3.52 | |
| | | yl)phenyl)ethyl)pyrrolidine- | (m, 5H), 3.05-2.99 (m, | |
| | | 2-carboxamide | 2H), 2.82-2.77 (m, 1H), | |
| | | | 2.45 (s, 3H), 2.30-2.21 | |
| | | | (m, 2H), 2.19-2.08 (m, | |
| | | | 3H), 2.05-1.98 (m, 1H), | |
| | | | 1.93-1.80 (m, 2H), 1.73- | |
| | | | 1.55 (m, 8H), 1.44-1.39 | |
| | | | (m, 1H), 0.91 (s, 9H) | |
| 89 | Ex. 24 | (2S,4R)-1-((2S)-2-(5- | ¹H NMR (400 MHz, | 1108.5 |
| | | ((2S)-2-(((7-(2-amino-3- | DMSO-d₆) δ: 9.00-8.95 | |
| | | cyano-7- | (m, 1H), 8.37 (d, J = 7.6 | |
| | | fluorobenzo[b]thiophen- | Hz, 1H), 8.27 (s, 1H), 8.14- | |
| | | 4-yl)-4-(3,8- | 8.07 (m, 2H), 7.87-7.80 | |
| | | diazabicyclo[3.2.1]octan- | (m, 1H), 7.76 (d, J = 9.6 | |
| | | 3-yl)-6-chloro-8- | Hz, 1H), 7.46-7.35 (m, | |
| | | fluoroquinazolin-2- | 4H), 7.26 (dd, J = 5.1, 8.2 | |
| | | yl)oxy)methyl)pyrrolidin- | Hz, 1H), 7.14 (t, J = 8.8 | |
| | | 1-yl)pentanamido)- | Hz, 1H), 4.95-4.86 (m, | |
| | | 3,3-dimethylbutanoyl)- | 1H), 4.50 (d, J = 9.6 Hz, | |
| | | 4-hydroxy-N-((S)-1-(4- | 1H), 4.44-4.26 (m, 6H), | |
| | | (4-methylthiazol-5- | 4.09-4.00 (m, 2H), 3.05- | |
| | | yl)phenyl)ethyl)pyrrolidine- | 2.99 (m, 2H), 2.85-2.76 | |
| | | 2-carboxamide | (m, 4H), 2.45 (s, 3H), 2.30- | |
| | | (2S,4R)-1-((2S)-2-(5- | 2.22 (m, 2H), 2.18-2.06 | |
| | | | (m, 3H), 2.03-1.97 (m, | |
| | | | 1H), 1.92-1.87 (m, 1H), | |
| | | | 1.83-1.74 (m, 2H), 1.73- | |
| | | | 1.57 (m, 8H), 1.45-1.34 | |
| | | | (m, 6H), 0.90 (d, J = 2.4 | |
| | | | Hz, 9H) | |
| 90 | Ex. 24 | ((2S)-2-(((7-(2-amino-3- | ¹H NMR (400 MHz, | 1142.6 |
| | | cyano-7- | DMSO-d₆) δ: 8.98 (s, 1H), | |
| | | fluorobenzo[b] thiophen- | 8.37 (d, J = 7.6 Hz, 1H), | |
| | | 4-yl)-4-(3,8- | 8.20 (s, 2H), 8.06 (s, 3H), | |
| | | diazabicyclo[3.2.1]octan- | 7.76 (d, J = 9.2 Hz, 1H), | |
| | | 3-yl)-8-fluoro-6- | 7.45-7.35 (m, 4H), 7.29- | |
| | | (trifluoromethyl)quinazolin- | 7.23 (m, 1H), 7.13 (t, J = | |
| | | 2- | 8.8 Hz, 1H), 4.91 (m, J = | |
| | | yl)oxy)methyl)pyrrolidin- | 7.2 Hz, 1H), 4.54-4.49 | |
| | | 1-yl)pentanamido)- | (m, 1H), 4.45-4.25 (m, | |
| | | 3,3-dimethylbutanoyl)- | 6H), 4.13-4.06 (m, 1H), | |
| | | 4-hydroxy-N-((S)-1-(4- | 3.65-3.58 (m, 6H), 3.07- | |
| | | (4-methylthiazol-5- | 2.99 (m, 1H), 2.83 (d, J = | |
| | | yl)phenyl)ethyl)pyrrolidine- | 7.6 Hz, 2H), 2.45 (s, 3H), | |
| | | 2-carboxamide | 2.35-2.22 (m, 2H), 2.19- | |
| | | | 2.07 (m, 2H), 2.04-1.97 | |
| | | | (m, 1H), 1.94-1.88 (m, | |
| | | | 1H), 1.82-1.76 (m, 1H), | |
| | | | 1.73-1.54 (m, 8H), 1.47- | |
| | | | 1.40 (m, 3H), 1.36 (d, J = | |
| | | | 6.8 Hz, 3H), 0.90 (d, J = | |
| | | | 3.2 Hz, 9H) | |

*Indicates that the method of preparation is the same as the synthetic procedure of the example compound.

Example 91: (2S,4R)-1-((2S)-2-(3-(3-((2S)-2-((((7S)-7-(2-amino-3-cyano-7-fluorobenzo[b]thiophen-4-yl)-4-(3,8-diazabicyclo[3.2.1]octan-3-yl)-8-fluoro-6-(trifluoromethyl)quinazolin-2-yl)oxy)methyl)pyrrolidin-1-yl)propoxy)propanamido)-3,3-dimethylbutanoyl)-4-hydroxy-N—((S)-1-(4-(4-methylthiazol-5-yl)phenyl)ethyl)pyrrolidine-2-carboxamide

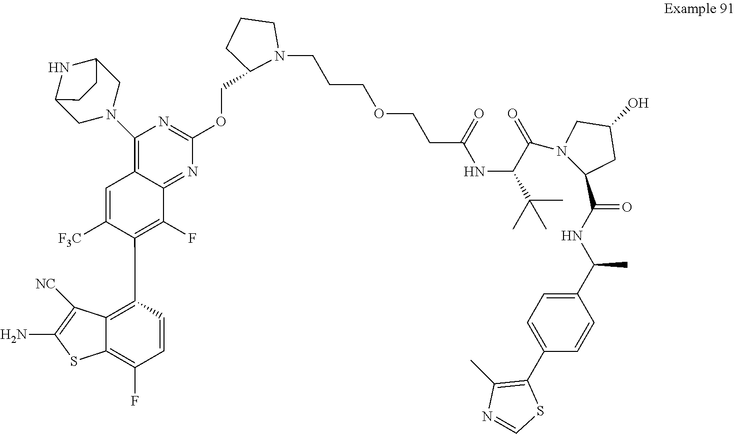

LCMS (ESI, m/z): 1172.8. ¹H NMR (400 MHz, DMSO-d₆) δ: 8.98 (s, 1H), 8.39 (d, J=7.6 Hz, 1H), 8.20-8.19 (m, 1H), 8.07 (br d, J=7.6 Hz, 3H), 7.82 (d, J=9.2 Hz, 1H), 7.45-7.42 (m, 2H), 7.40-7.36 (m, 2H), 7.26 (dd, J=5.4, 8.4 Hz, 1H), 7.16-7.10 (m, 1H), 4.90 (br t, J=7.2 Hz, 1H), 4.51 (d, J=9.2 Hz, 1H), 4.44-4.36 (m, 2H), 4.35-4.25 (m, 4H), 4.09 (br dd, J=7.8, 10.8 Hz, 1H), 3.58 (br s, 8H), 3.09-2.99 (m, 2H), 2.94-2.75 (m, 3H), 2.27 (td, J=5.6, 14.4 Hz, 2H), 2.22-2.13 (m, 2H), 2.04-1.86 (m, 3H), 1.83-1.72 (m, 2H), 1.72-1.58 (m, 1OH), 1.54 (br s, 1H), 1.36 (d, J=7.2 Hz, 3H), 0.90 (s, 9H).

Example 92: (2S,4R)-1-((2S)-2-(3-(3-((2S)-2-((((7S)-7-(2-amino-3-cyano-7-fluorobenzo[b]thiophen-4-yl)-4-(3,8-diazabicyclo[3.2.1]octan-3-yl)-8-fluoro-6-(trifluoromethyl)quinazolin-2-yl)oxy)methyl)pyrrolidin-1-yl)propoxy)propanamido)-3,3-dimethylbutanoyl)-4-hydroxy-N—((S)-1-(2′,4′,6′-trifluoro-[1,1′-biphenyl]-4-yl)ethyl)pyrrolidine-2-carboxamide

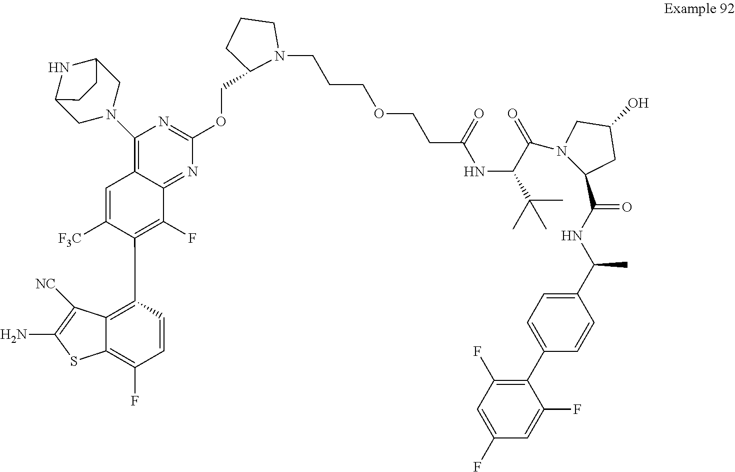

LCMS (ESI, m/z): 1205.5. ¹H NMR (400 MHz, DMSO-d₆) δ: 8.37 (d, J=7.6 Hz, 1H), 8.16 (s, 1H), 8.06 (s, 3H), 7.81 (d, J=10.0 Hz, 1H), 7.38 (d, J=5.6 Hz, 4H), 7.33-7.29 (m, 2H), 7.25 (dd, J=5.6, 8.4 Hz, 1H), 7.16-7.10 (m, 1H), 4.96-4.89 (m, 1H), 4.54-4.49 (m, 1H), 4.44-4.41 (m, 1H), 4.40-4.32 (m, 4H), 4.28 (d, J=5.2 Hz, 1H), 4.11 (dd, J=7.2, 10.4 Hz, 1H), 3.69-3.57 (m, 8H), 3.06 (d, J=2.4 Hz, 1H), 2.86 (dd, J=4.0, 14.4 Hz, 3H), 2.27-2.17 (m, 4H), 2.02-1.98 (m, 1H), 1.93-1.87 (m, 2H), 1.80 (d, J=4.4 Hz, 1H), 1.73-1.62 (m, 10H), 1.37 (d, J=7.2 Hz, 3H), 0.90 (s, 9H).

Example 93: (2S,4R)-1-((2S)-2-(3-(3-((2S)-2-((((7S)-7-(2-amino-3-cyano-7-fluorobenzo [b]thiophen-4-yl)-4-(3,8-diazabicyclo [3.2.1]octan-3-yl)-8-fluoro-6-(trifluoromethyl)quinazolin-2-yl)oxy)methyl)pyrrolidin-1-yl)propoxy)propanamido)-3,3-dimethylbutanoyl)-4-hydroxy-N—((S)-1-(2′,3′,6′-trifluoro-[1,1′-biphenyl]-4-yl)ethyl)pyrrolidine-2-carboxamide

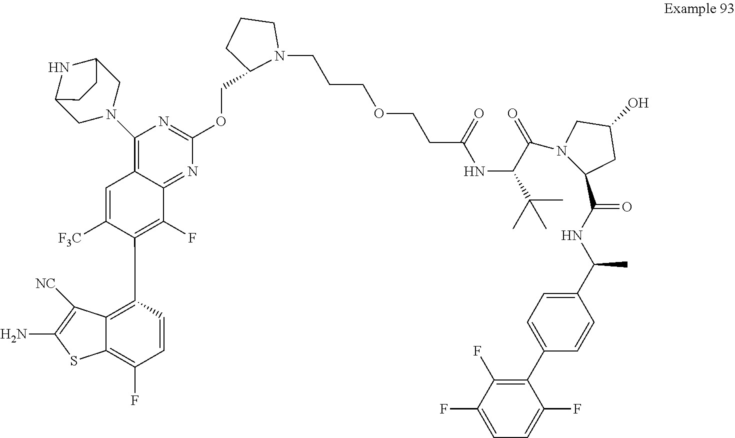

LCMS (ESI, m/z): 1205.5. ¹H NMR (400 MHz, DMSO-d₆) δ: 8.39 (d, J=7.6 Hz, 1H), 8.19 (s, 1H), 8.13-8.02 (m, 3H), 7.81 (d, J=9.2 Hz, 1H), 7.59-7.49 (m, 1H), 7.42 (s, 4H), 7.29-7.22 (m, 2H), 7.17-7.09 (m, 1H), 4.98-4.90 (m, 1H), 4.56-4.50 (m, 1H), 4.42 (t, J=8.0 Hz, 1H), 4.36 (dd, J=4.0, 10.8 Hz, 2H), 4.32-4.27 (m, 2H), 4.08 (dd, J=7.2, 10.8 Hz, 1H), 3.59 (d, J=6.8 Hz, 8H), 3.05-3.01 (m, 1H), 2.91-2.86 (m, 1H), 2.85-2.78 (m, 2H), 2.27 (td, J=5.6, 14.8 Hz, 2H), 2.22-2.14 (m, 2H), 2.05-1.99 (m, 1H), 1.92-1.75 (m, 3H), 1.74-1.57 (m, 10H), 1.38 (d, J=6.8 Hz, 3H), 0.90 (s, 9H).

Example 94: (2S,4R)-1-((2S)-2-(3-(3-((2S)-2-((((7S)-7-(2-amino-3-cyano-7-fluorobenzo [b]thiophen-4-yl)-4-(3,8-diazabicyclo[3.2.1]octan-3-yl)-8-fluoro-6-(trifluoromethyl)quinazolin-2-yl)oxy)methyl)pyrrolidin-1-yl)propoxy)propanamido)-3,3-dimethylbutanoyl)-N—((S)-1-(2′,3′-difluoro-[1,1′-biphenyl]-4-yl)ethyl)-4-hydroxypyrrolidine-2-carboxamide

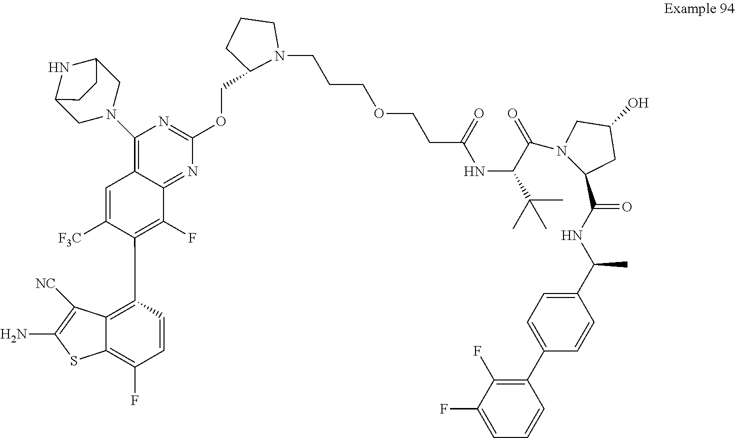

LCMS (ESI, m/z): 1187.6. ¹H NMR (400 MHz, DMSO-d₆) δ: 8.41-8.37 (m, 1H), 8.24 (s, 1H), 8.06 (d, J=5.2 Hz, 3H), 7.81 (d, J=9.2 Hz, 1H), 7.54-7.48 (m, 2H), 7.47-7.23 (m, 7H), 7.15-7.09 (m, 1H), 4.96-4.89 (m, 1H), 4.51 (d, J=9.2 Hz, 1H), 4.44-4.41 (m, 1H), 4.40-4.34 (m, 2H), 4.28 (d, J=13.6 Hz, 2H), 4.12-4.05 (m, 2H), 3.56 (d, J=11.2 Hz, 8H), 3.05-3.02 (m, 2H), 2.81-2.77 (m, 2H), 2.30-2.23 (m, 2H), 2.20-2.13 (m, 2H), 2.00 (d, J=3.2 Hz, 1H), 1.89 (d, J=4.0 Hz, 1H), 1.80 (dd, J=4.4, 8.0 Hz, 1H), 1.73-1.57 (m, 1OH), 1.37 (d, J=6.8 Hz, 3H), 0.90 (s, 9H).

Example 95: (2S,4R)-1-((2S)-2-(3-(3-((2S)-2-((((7S)-7-(2-amino-3-cyano-7-fluorobenzo [b]thiophen-4-yl)-4-(3,8-diazabicyclo [3.2.1]octan-3-yl)-8-fluoro-6-(trifluoromethyl)quinazolin-2-yl)oxy)methyl)pyrrolidin-1-yl)propoxy)propanamido)-3,3-dimethylbutanoyl)-N—((S)-1-(2′-chloro-[1,1′-biphenyl]-4-yl)ethyl)-4-hydroxypyrrolidine-2-carboxamide

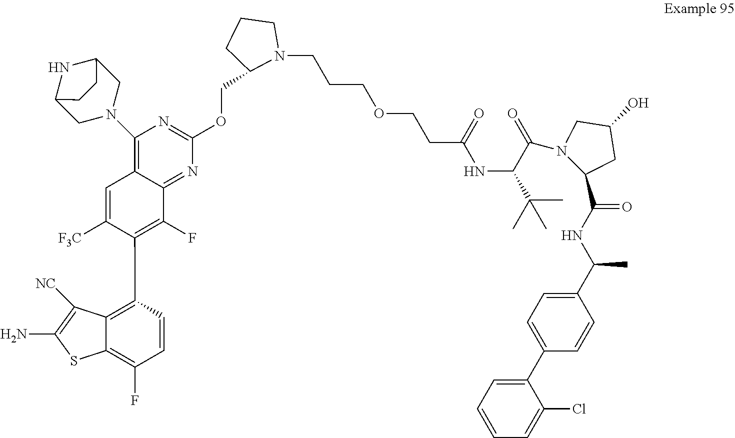

LCMS (ESI, m/z): 1185.8. ¹H NMR (400 MHz, DMSO-d₆) δ: 8.41-8.35 (m, 1H), 8.19 (s, 1H), 8.07 (br s, 3H), 7.84 (s, 1H), 7.82-7.80 (m, 1H), 7.59-7.52 (m, 1H), 7.53-7.51 (m, 1H), 7.43-7.35 (m, 8H), 7.29-7.23 (m, 1H), 7.12 (s, 1H), 7.10 (br d, J=1.2 Hz, 1H), 4.98-4.92 (m, 1H), 5.01 (s, 1H), 4.56-4.50 (m, 1H), 4.43 (s, 1H), 4.34 (br d, J=4.4 Hz, 1H), 4.28 (br s, 2H), 4.14-4.05 (m, 1H), 3.61 (br d, J=2.4 Hz, 1H), 3.59 (br s, 1H), 3.56 (br s, 3H), 3.51-3.47 (m, 2H), 3.06-3.02 (m, 1H), 2.91-2.75 (m, 2H), 2.30-2.26 (m, 1H), 2.22-2.13 (m, 2H), 2.09-1.97 (m, 2H), 1.93-1.76 (m, 3H), 1.72-1.55 (m, 10H), 1.39 (d, J=7.2 Hz, 3H), 0.92 (s, 7H).

Example 96: (2S,4R)-1-((2S)-2-(3-(3-((2S)-2-((((7R)-7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)pyrrolidin-1-yl)propoxy)propanamido)-3,3-dimethylbutanoyl)-N—((S)-1-(2′-chloro-[1,1′-biphenyl]-4-yl)ethyl)-4-hydroxypyrrolidine-2-carboxamide

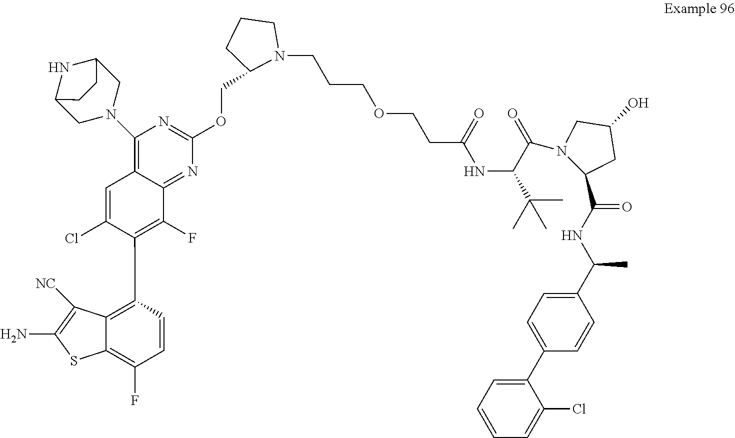

LCMS (ESI, m/z): 1153.4. ¹H NMR (400 MHz, DMSO-d₆) δ: 8.30 (d, J=7.8 Hz, 1H), 8.16 (s, 1H), 8.06 (s, 2H), 7.87 (s, 1H), 7.76 (d, J=9.2 Hz, 1H), 7.58-7.53 (m, 1H), 7.43-7.37 (m, 7H), 7.25 (dd, J=5.2, 8.4 Hz, 1H), 7.18-7.12 (m, 1H), 5.00-4.90 (m, 1H), 4.53 (d, J=9.2 Hz, 1H), 4.45 (t, J=8.0 Hz, 1H), 4.40-4.29 (m, 4H), 4.13 (br s, 1H), 3.79 (br s, 2H), 3.69-3.61 (m, 4H), 3.60-3.53 (m, 5H), 3.12-3.04 (m, 3H), 2.96-2.85 (m, 3H), 2.46-2.38 (m, 2H), 2.31-2.23 (m, 2H), 2.05-1.99 (m, 1H), 1.96-1.90 (m, 1H), 1.89-1.81 (m, 1H), 1.78-1.68 (m, 7H), 1.40 (d, J=6.8 Hz, 3H), 0.93 (s, 9H).

Example 97: (2S,4R)-1-((2S)-2-(3-(3-((2S)-2-((((7R)-7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)methyl)pyrrolidin-1-yl)propoxy)propanamido)-3,3-dimethylbutanoyl)-4-hydroxy-N—((S)-1-(2′,4′,6′-trifluoro-[1,1′-biphenyl]-4-yl)ethyl)pyrrolidine-2-carboxamide

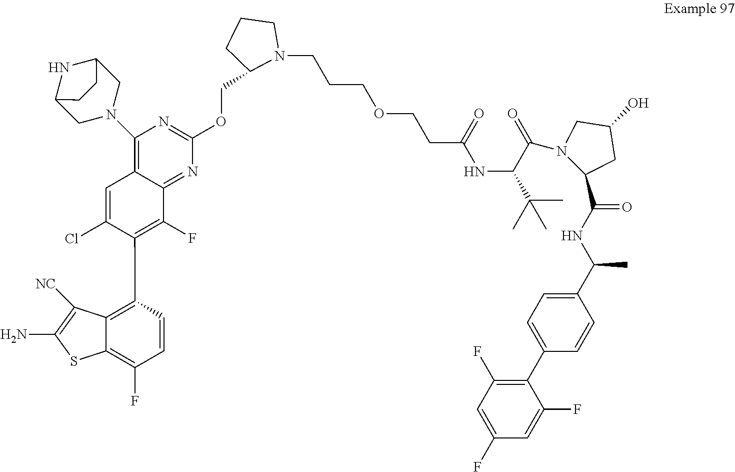

LCMS (ESI, m/z): 1171.4. ¹H NMR (400 MHz, DMSO-d₆) δ: 8.34 (d, J=8.0 Hz, 1H), 8.15 (s, 1H), 8.08 (s, 2H), 7.89 (s, 1H), 7.81-7.75 (m, 1H), 7.43-7.35 (m, 5H), 7.33-7.28 (m, 2H), 7.25 (dd, J=5.2, 8.4 Hz, 1H), 7.18-7.13 (m, 1H), 4.98-4.89 (m, 1H), 4.56-4.50 (m, 1H), 4.47-4.39 (m, 3H), 4.38-4.27 (m, 3H), 4.16 (br dd, J=6.8, 11.1 Hz, 1H), 3.94 (br dd, J=2.4, 4.8 Hz, 3H), 3.77-3.71 (m, 2H), 3.67-3.59 (m, 5H), 3.14-3.06 (m, 4H), 2.99-2.90 (m, 3H), 2.32-2.25 (m, 2H), 2.06-1.90 (m, 3H), 1.85-1.79 (m, 4H), 1.73-1.65 (m, 4H), 1.39 (d, J=7.2 Hz, 3H), 0.92 (s, 9H).

Example 98: (2S,4R)-1-((2R)-2-(3-(7-(2-((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)ethyl)-2,7-diazaspiro[3.5]nonan-2-yl)isoxazol-5-yl)-3-methylbutanoyl)-N—((S)-1-(2′,3′-difluoro-[1,1′-biphenyl]-4-yl)ethyl)-4-hydroxypyrrolidine-2-carboxamide

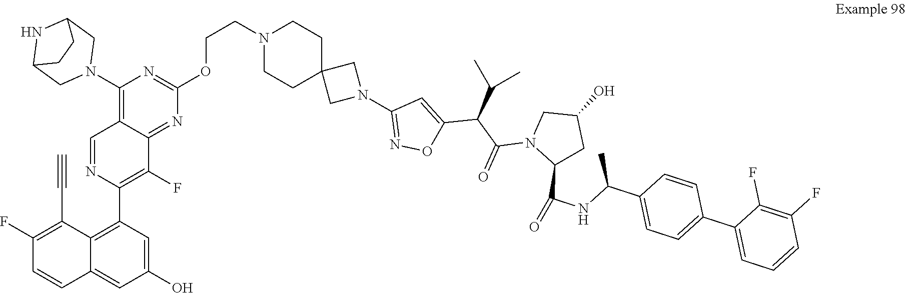 [SMILES: C#Cc1c(F)ccc2cc(O)cc(-c3ncc4c(N5CC6CCC(C5)N6)nc(OCCN5CCC6(CC5)CN(c5cc([C@H](C(=O)N7C[C@H](O)C[C@H]7C(=O)N[C@@H](C)c7ccc(-c8cccc(F)c8F)cc7)C(C)C)on5)C6)nc4c3F)c12]

LCMS (ESI, m/z): 1107.4. ¹H NMR (400 MHz, DMSO-d₆) δ: 9.09-8.98 (m, 1H), 8.46-8.31 (m, 1H), 8.16 (s, 1H), 8.07-7.91 (m, 1H), 7.56-7.46 (m, 10H), 7.20-7.14 (m, 1H), 5.89-5.76 (m, 1H), 5.00-4.87 (m, 1H), 4.56-4.27 (m, 6H), 3.96-3.91 (m, 1H), 3.74-3.63 (m, 5H), 3.63-3.53 (m, 9H), 2.46-2.41 (m, 6H), 2.06-1.97 (m, 2H), 1.78-1.66 (m, 8H), 1.42-1.35 (m, 3H), 0.98-0.91 (m, 3H), 0.83-0.75 (m, 3H).

Example 99: (2S,4R)-1-((2R)-2-(3-(7-(2-((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)ethyl)-2,7-diazaspiro[3.5]nonan-2-yl)isoxazol-5-yl)-3-methylbutanoyl)-N—((S)-1-(2′-chloro-[1,1′-biphenyl]-4-yl)ethyl)-4-hydroxypyrrolidine-2-carboxamide

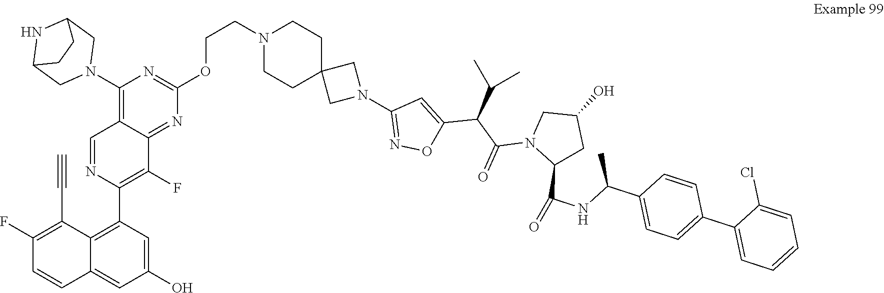 [SMILES: C#Cc1c(F)ccc2cc(O)cc(-c3ncc4c(N5CC6CCC(C5)N6)nc(OCCN5CCC6(CC5)CN(c5cc([C@H](C(=O)N7C[C@H](O)C[C@H]7C(=O)N[C@@H](C)c7ccc(-c8ccccc8Cl)cc7)C(C)C)on5)C6)nc4c3F)c12]

LCMS (ESI, m/z): 1105.1. ¹H NMR (400 MHz, DMSO-d₆) δ:10.35-10.23 (m, 1H), 9.29-9.18 (m, 1H), 8.54-8.39 (m, 1H), 8.06 (s, 1H), 7.69-7.62 (m, 1H), 7.58-7.42 (m, 10H), 5.96-5.93 (m, 1H), 5.23-5.16 (m, 1H), 5.07-4.99 (m, 1H), 4.93-4.71 (m, 4H), 4.46-4.25 (m, 4H), 4.05-3.92 (m, 3H), 3.85-3.76 (m, 3H), 3.73-3.66 (m, 8H), 2.37-2.25 (m, 3H), 2.24-1.90 (m, 11H), 1.49 (d, J=7.2 Hz, 3H), 1.08-1.04 (m, 3H), 0.88 (br d, J=6.8 Hz, 3H).

Example 100: (2S,4R)-1-((2R)-2-(3-((1-(2-((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)ethyl)piperidin-4-yl)oxy)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide

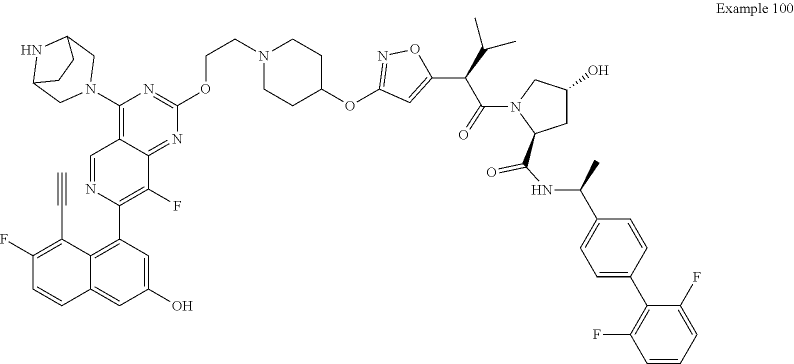 [SMILES: C#Cc1c(F)ccc2cc(O)cc(-c3ncc4c(N5CC6CCC(C5)N6)nc(OCCN5CCC(Oc6cc([C@H](C(=O)N7C[C@H](O)C[C@H]7C(=O)N[C@@H](C)c7ccc(-c8c(F)cccc8F)cc7)C(C)C)on6)CC5)nc4c3F)c12]

LCMS (ESI, m/z): 1082.8. ¹H NMR (400 MHz, DMSO-d₆) δ:10.19 (s, 1H), 9.10 (s, 1H), 8.42 (d, J=8.0 Hz, 1H), 7.99 (dd, J=6.0, 9.2 Hz, 1H), 7.50 (br s, 2H), 7.41-7.38 (m, 5H), 7.24-7.18 (m, 3H), 6.09 (s, 1H), 5.11 (d, J=3.6 Hz, 1H), 4.93 (t, J=7.2 Hz, 1H), 4.65 (br d, J=13.6 Hz, 1H), 4.59-4.43 (m, 5H), 4.37 (t, J=8.0 Hz, 1H), 4.29 (br d, J=4.0 Hz, 1H), 4.14 (br s, 2H), 3.95 (s, 1H), 3.80 (br dd, J=9.2, 12.4 Hz, 2H), 3.73-3.68 (m, 1H), 3.65 (d, J=10.0 Hz, 1H), 3.48-3.43 (m, 2H), 2.81 (br s, 2H), 2.26-2.16 (m, 2H), 2.10-1.85 (m, 9H), 1.82-1.76 (m, 1H), 1.73-1.66 (m, 2H), 1.46 (br d, J=7.2 Hz, 3H), 0.99-0.95 (m, 3H), 0.84-0.78 (m, 3H).

Example 101: (2S,4R)-1-((2R)-2-(3-(6-(2-((4-(3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)ethyl)-2,6-diazaspiro[3.4]octan-2-yl)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide

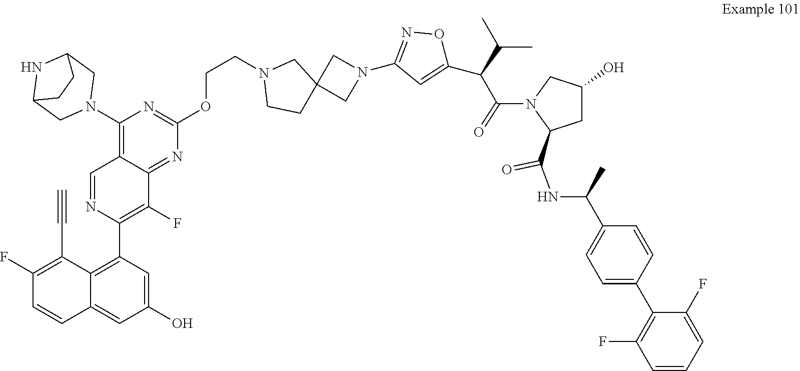 [SMILES: C#Cc1c(F)ccc2cc(O)cc(-c3ncc4c(N5CC6CCC(C5)N6)nc(OCCN5CCC6(C5)CN(c5cc([C@H](C(=O)N7C[C@H](O)C[C@H]7C(=O)N[C@@H](C)c7ccc(-c8c(F)cccc8F)cc7)C(C)C)on5)C6)nc4c3F)c12]

LCMS (ESI, m/z): 1093.8. ¹H NMR (400 MHz, DMSO-d₆) δ 10.17 (s, 1H), 9.08 (s, 1H), 8.40 (d, J=7.6 Hz, 1H), 7.98 (dd, J=5.6, 9.2 Hz, 1H), 7.49-7.44 (m, 2H), 7.41-7.37 (m, 5H), 7.25-7.16 (m, 3H), 5.75 (s, 1H), 5.14-5.06 (m, 1H), 4.97 (s, 1H), 4.64-4.56 (m, 1H), 4.52-4.40 (m, 3H), 4.39-4.33 (m, 1H), 4.28 (br d, J=3.2 Hz, 1H), 4.03-3.89 (m, 3H), 3.69 (br d, J=3.2 Hz, 7H), 3.59-3.55 (m, 1H), 3.40 (br d, J=10.0 Hz, 2H), 2.89 (br d, J=1.6 Hz, 4H), 2.64-2.60 (m, 1H), 2.16 (br d, J=2.8 Hz, 2H), 2.06 (br s, 3H), 1.90-1.75 (m, 5H), 1.48-1.37 (m, 3H), 0.94 (br d, J=6.4 Hz, 3H), 0.83-0.75 (m, 3H).

G12C Degrader Experimental Procedures

Example 200: (2S,4R)-1-((R)-2-(3-((1-(2-((4-((S)-4-acryloyl-2-methylpiperazin-1-yl)-7-((R)-6-amino-4-methyl-3-(trifluoromethyl)pyridin-2-yl)-6-chloro-8-fluoroquinazolin-2-yl)oxy)ethyl)piperidin-4-yl)methoxy)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide

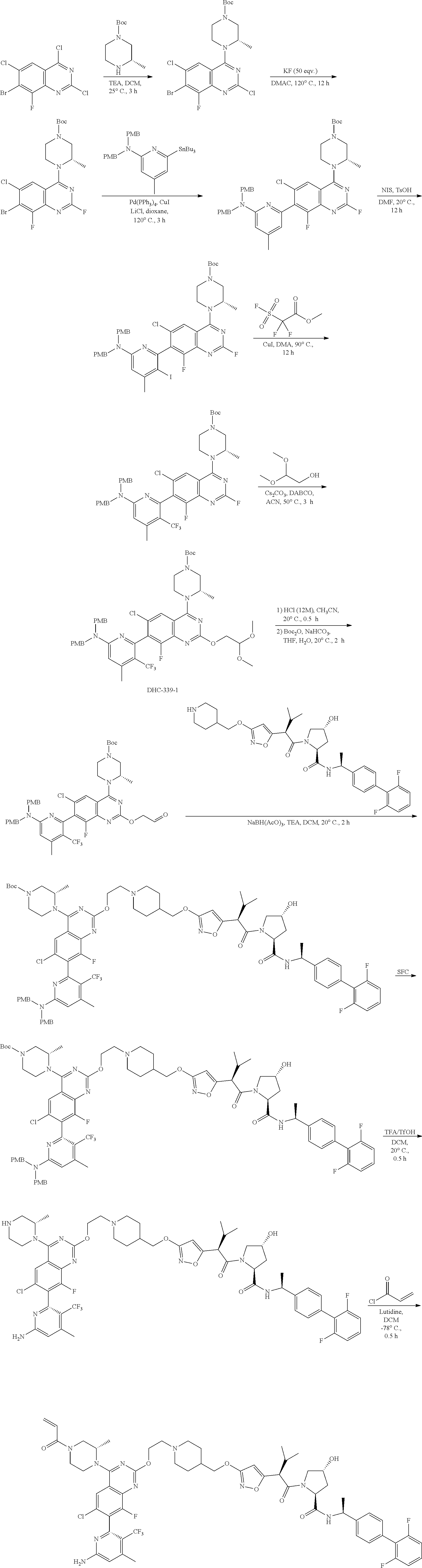

Step 1 tert-butyl (S)-4-(7-bromo-2,6-dichloro-8-fluoroquinazolin-4-yl)-3-methylpiperazine-1-carboxylate

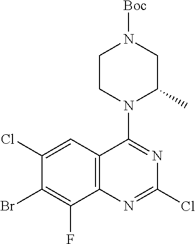 [SMILES: C[C@H]1CN(C(=O)OC(C)(C)C)CCN1c1nc(Cl)nc2c(F)c(Br)c(Cl)cc12]

To a solution of 7-bromo-2,4,6-trichloro-8-fluoroquinazoline (1 g, 3.03 mmol, 1 eq) and tert-butyl (S)-3-methylpiperazine-1-carboxylate (667 mg, 3.33 mmol, 1.1 eq) in dichloromethane (10 mL) was added triethylamine (613 mg, 6.05 mmol, 2 eq). Then the mixture was stirred at 20° C. for 0.5 h. TLC (petroleum ether/ethyl acetate=5/1) indicated the starting material was consumed completely and a new spot was formed. The mixture was diluted with water (30 mL) and extracted by dichloromethane (30 mL×3). The combined organic layers were washed with brine (20 mL×2), dried over sodium sulfate, filtered and then concentrated under vacuum to get a residue. The residue was purified by column chromatography (silicon dioxide, petroleum ether/tetrahydrofuran=100/1 to 12/1) to afford the desired product tert-butyl (S)-4-(7-bromo-2,6-dichloro-8-fluoroquinazolin-4-yl)-3-methylpiperazine-1-carboxylate (1.4 g, 2.83 mmol, 93% yield) as a white solid. ¹H NMR (400 MHz, CDCl₃) δ 7.98 (s, 1H), 4.77-4.76 (m, 1H), 4.17-4.14 (m, 1H), 3.92-3.72 (m, 3H), 3.29-3.06 (m, 2H), 1.43 (s, 9H), 1.33 (d, J=6.8 Hz, 3H).

Step 2 tert-butyl (S)-4-(7-bromo-6-chloro-2,8-difluoroquinazolin-4-yl)-3-methylpiperazine-1-carboxylate

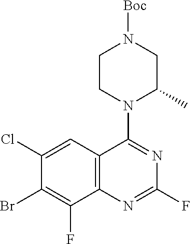 [SMILES: C[C@H]1CN(C(=O)OC(C)(C)C)CCN1c1nc(F)nc2c(F)c(Br)c(Cl)cc12]

To a solution of tert-butyl (S)-4-(7-bromo-2,6-dichloro-8-fluoroquinazolin-4-yl)-3-methylpiperazine-1-carboxylate (3.5 g, 7.08 mmol, 1 eq) in dimethylacetamide (100 mL) was added potassium fluoride (20.57 g, 354.12 mmol, 50 eq). Then the mixture was stirred at 120° C. for 12 h. LCMS showed the desired mass was detected. The mixture was diluted with water (500 mL), extracted with ethyl acetate (500 mL×3). The combined organic layers were washed with brine (200 mL×3), dried over sodium sulfate, filtered and then concentrated under reduced pressure to get a residue. The residue was purified by column chromatography (silicon dioxide, petroleum ether/ethyl acetate=20/1 to 8/1) to afford the desired product tert-butyl (S)-4-(7-bromo-6-chloro-2,8-difluoroquinazolin-4-yl)-3-methylpiperazine-1-carboxylate (2.65 g, 5.55 mmol, 78% yield) as a yellow solid. LCMS (ESI) m/z: 477.0 [M+H]⁺. ¹H NMR (400 MHz, CDCl₃) (7.75 (d, J=1.6 Hz, 1H), 4.80-4.65 (m, 1H), 4.24 (d, J=13.6 Hz, 1H), 4.04-3.87 (m, 1H), 3.66 (t, J=11.6 Hz, 1H), 3.37-2.97 (m, 2H), 1.52-1.47 (m, 13H).

Step 3 tert-butyl (S)-4-(7-(6-(bis(4-methoxybenzyl)amino)-4-methylpyridin-2-yl)-6-chloro-2,8-difluoroquinazolin-4-yl)-3-methylpiperazine-1-carboxylate

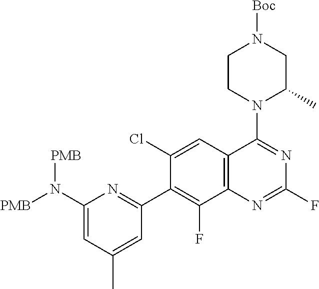

To a solution of tert-butyl (S)-4-(7-bromo-6-chloro-2,8-difluoroquinazolin-4-yl)-3-methylpiperazine-1-carboxylate (3 g, 6.28 mmol, 1 eq) and N,N-bis(4-methoxybenzyl)-4-methyl-6-(tributylstannyl)pyridin-2-amine (8.01 g, 12.56 mmol, 2 eq) in dioxane (10 mL) was added cuprous iodide (240 mg, 1.24 mmol, 0.2 eq) and lithium chloride (666 mg, 15.7 mmol, 2.5 eq). The mixture was purged with nitrogen for 3 times, then tetrakis[triphenylphosphine] palladium (726 mg, 0.63 mmol, 0.1 eq) was added into the mixture and stirred at 120° C. for 3 h. TLC (petroleum ether/ethyl acetate=3/1) showed tert-butyl (3S)-4-(7-bromo-6-chloro-2,8-difluoro-quinazolin-4-yl)-3-methyl-piperazine-1-carboxylate was consumed completely and several new spots were detected. The mixture was filtered and then the filtrate was concentrated under reduced pressure to get a residue. The residue was purified by column chromatography (silicon dioxide, petroleum ether/ethyl acetate=10/1 to 3/1) to afford the product tert-butyl (S)-4-(7-(6-(bis(4-methoxybenzyl)amino)-4-methylpyridin-2-yl)-6-chloro-2,8-difluoroquinazolin-4-yl)-3-methylpiperazine-1-carboxylate (2.9 g, 3.89 mmol, 62% yield) as a yellow solid. ¹H NMR (400 MHz, CDCl₃) δ 7.72 (s, 1H), 7.28 (s, 2H), 7.20 (d, J=8.8 Hz, 4H), 6.87 (d, J=8.8 Hz, 4H), 6.61 (s, 1H), 6.39 (s, 1H), 5.32 (s, 1H), 4.83-4.67 (m, 5H), 4.28 (d, J=13.2 Hz, 1H), 3.82 (s, 6H), 3.35-3.04 (m, 2H), 2.29 (s, 3H), 1.54-1.47 (m, 12H).

Step 4 tert-butyl (3S)-4-(7-(6-(bis(4-methoxybenzyl)amino)-3-iodo-4-methylpyridin-2-yl)-6-chloro-2,8-difluoroquinazolin-4-yl)-3-methylpiperazine-1-carboxylate

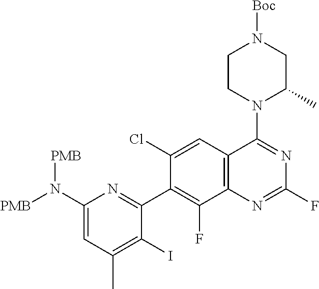

To a solution of tert-butyl (S)-4-(7-(6-(bis(4-methoxybenzyl)amino)-4-methylpyridin-2-yl)-6-chloro-2,8-difluoroquinazolin-4-yl)-3-methylpiperazine-1-carboxylate(1.3 g, 1.74 mmol, 1 eq) and N-iodosuccinimide (1.96 g, 8.72 mmol, 5 eq) in N,N-dimethylformamide (13 mL) was added p-toluenesulfonic acid (12 mg, 0.07 mmol, 0.04 eq). Then the mixture was stirred at 20° C. for 12 h. LCMS showed the desired mass was detected. The mixture was diluted with ethyl acetate (50 mL), quenched with saturated aqueous sodium sulfite (30 mL), extracted with ethyl acetate (30 mL×3). The combined organic layers were washed with brine (50 mL), dried over sodium sulfate, filtered and then concentrated under reduced pressure to get a residue. The residue was purified by column chromatography (silicon dioxide, petroleum ether/ethyl acetate=10/1 to 3/1) to afford the desired product tert-butyl (3S)-4-(7-(6-(bis(4-methoxybenzyl)amino)-3-iodo-4-methylpyridin-2-yl)-6-chloro-2,8-difluoroquinazolin-4-yl)-3-methylpiperazine-1-carboxylate (1.1 g, 1.26 mmol, 72% yield) as a yellow solid. LCMS (ESI) m/z: 871.4 [M+H]⁺. ¹H NMR (400 MHz, CDCl₃) (7.77 (s, 1H), 7.17 (d, J=8.8 Hz, 4H), 6.86 (d, J=8.8 Hz, 4H), 6.48 (s, 1H), 4.89-4.64 (m, 3H), 4.63-4.51 (m, 2H), 4.43-4.19 (m, 2H), 3.82 (s, 6H), 3.75-3.62 (m, 1H), 3.40-3.06 (m, 2H), 2.38 (s, 3H), 1.56-1.47 (m, 13H).

Step 5 tert-butyl (3S)-4-(7-(6-(bis(4-methoxybenzyl)amino)-4-methyl-3-(trifluoromethyl)pyridin-2-yl)-6-chloro-2,8-difluoroquinazolin-4-yl)-3-methylpiperazine-1-carboxylate

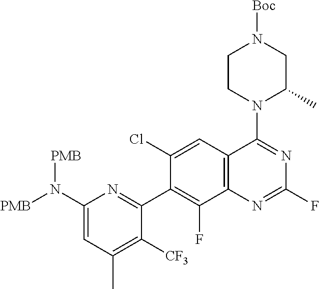

To a solution of tert-butyl (3S)-4-(7-(6-(bis(4-methoxybenzyl)amino)-3-iodo-4-methylpyridin-2-yl)-6-chloro-2,8-difluoroquinazolin-4-yl)-3-methylpiperazine-1-carboxylate (6.06 g, 31.57 mmol, 25 eq) in dimethylacetamide (4 mL) was added cuprous iodide (2.89 g, 15.15 mmol, 12 eq). Then the mixture was stirred at 90° C. for 12 h. LCMS showed the desired mass was detected. The mixture was filtered, diluted with water (20 mL), extracted with ethyl acetate (20 mL×3). The combined organic layers were washed with brine (20 mL), dried over sodium sulfate, filtered and then concentrated under vacuum to get a residue. The residue was purified by prep-TLC (silicon dioxide, petroleum ether/ethyl acetate=3/1) to afford the product tert-butyl (3S)-4-(7-(6-(bis(4-methoxybenzyl)amino)-4-methyl-3-(trifluoromethyl)pyridin-2-yl)-6-chloro-2,8-difluoroquinazolin-4-yl)-3-methylpiperazine-1-carboxylate (900 mg, 1.11 mmol, 87% yield) as a yellow solid. LCMS (ESI) m z: 813.6 [M+H]⁺. ¹H NM (400 MHz, CDCl₃) δ 7.73 (s, 1H), 7.16 (d, J=8.8 Hz, 4H), 6.87 (d, J=8.8 Hz, 4H), 6.44 (s, 1H), 4.85-4.69 (m, 3H), 4.62-4.53 (m, 2H), 4.37-4.18 (m, 2H), 3.82 (s, 6H), 3.75-3.62 (m, 1H), 3.36-3.10 (m, 2H), 2.44 (d, J=1.2 Hz, 3H), 1.54-1.48 (m, 13H).

Step 6: tert-butyl (3S)-4-(7-(6-(bis(4-methoxybenzyl)amino)-4-methyl-3-(trifluoromethyl) pyridin-2-yl)-6-chloro-2-(2,2-dimethoxyethoxy)-8-fluoroquinazolin-4-yl)-3-methylpiperazine-1-carboxylate

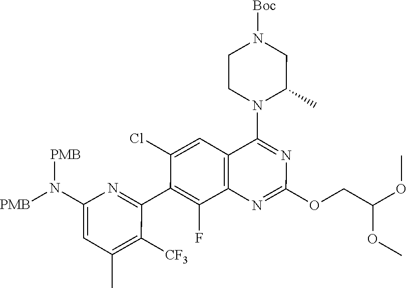

To a solution of 2,2-dimethoxyethan-1-ol (391 mg, 3.69 mmol, 1.5 eq) and tert-butyl (3S)-4-(7-(6-(bis(4-methoxybenzyl)amino)-4-methyl-3-(trifluoromethyl)pyridin-2-yl)-6-chloro-2,8-difluoroquinazolin-4-yl)-3-methylpiperazine-1-carboxylate (2 g, 2.46 mmol, 1 eq) in acetonitrile (20 mL) was added cesium carbonate (1.60 g, 4.92 mmol, 2 eq) and 1,4-diazabicyclo[2.2.2] octane (27 mg, 0.25 mmol, 0.1 eq). The mixture was stirred at 50° C. for 3 h. LCMS showed the desired mass was detected. The reaction mixture was filtered and concentrated under reduced pressure to give a residue. The residue was purified by flash silica gel chromatography (ISCO®; 12 g SepaFlash® Silica Flash Column, Eluent of 0-30% Ethyl acetate/Petroleum ethergradient @80 mL/min) to afford the product tert-butyl (3S)-4-(7-(6-(bis(4-methoxybenzyl)amino)-4-methyl-3-(trifluoromethyl)pyridin-2-yl)-6-chloro-2-(2,2-dimethoxyethoxy)-8-fluoroquinazolin-4-yl)-3-methylpiperazine-1-carboxylate (1.7 g, 1.89 mmol, 77% yield) as a yellow solid. LCMS (ESI, m/z): 899.4 [M+H]⁺.

Step 7: tert-butyl (3S)-4-(7-(6-(bis(4-methoxybenzyl)amino)-4-methyl-3-(trifluoromethyl) pyridin-2-yl)-6-chloro-8-fluoro-2-(2-oxoethoxy)quinazolin-4-yl)-3-methylpiperazine-1-carboxylate

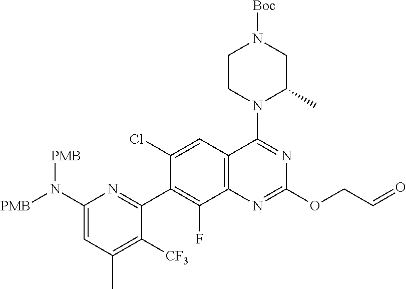

To a solution of tert-butyl (3S)-4-[7-[6-[bis[(4-methoxyphenyl)methyl]amino]-4-methyl-3-(trifluoromethyl)-2-pyridyl]-6-chloro-2-(2,2-dimethoxyethoxy)-8-fluoro-quinazolin-4-yl]-3-methyl-piperazine-1-carboxylate (500 mg, 0.56 mmol, 1 eq) in acetonitrile (10 mL) was added hydrochloric acid (12 M, 0.9 mL, 20 eq). The mixture was stirred at 20° C. for 0.5 h. Then the mixture was concentrated under reduced pressure to get a residue. The residue was dissolved in tetrahydrofuran (5 mL) and water (5 mL), then di-tert-butyl dicarbonate (255 mg, 1.17 mmol, 2.2 eq) and sodium bicarbonate (892 mg, 10.62 mmol, 20 eq) were added to the mixture. The mixture was stirred at 20° C. for 2 h. LCMS showed the desired mass was detected. The reaction mixture was diluted with water (10 mL) and extracted with ethyl acetate (10 mL×3). The combined organic layers were washed with brine (10 mL×3), dried over, filtered and concentrated under reduced pressure to give a residue. The residue was purified by prep-TLC (silicon oxide, dichloromethane/methanol=15/1) to afford the product tert-butyl (3S)-4-(7-(6-(bis(4-methoxybenzyl)amino)-4-methyl-3-(trifluoromethyl)pyridin-2-yl)-6-chloro-8-fluoro-2-(2-oxoethoxy)quinazolin-4-yl)-3-methylpiperazine-1-carboxylate (350 mg, 0.41 mmol, 77% yield) as a white solid. LCMS (ESI, m/z): 853.2 [M+H]⁺.

Step 10 tert-butyl (3S)-4-(7-(6-(bis(4-methoxybenzyl)amino)-4-methyl-3-(trifluoromethyl)pyridin-2-yl)-6-chloro-2-(2-(4-(((5-((R)-1-((2S,4R)-2-(((S)-1-(2′,6′-difluoro-[1,1′-biphenyl]-4-yl)ethyl) carbamoyl)-4-hydroxypyrrolidin-1-yl)-3-methyl-1-oxobutan-2-yl)isoxazol-3-yl)oxy)methyl) piperidin-1-yl)ethoxy)-8-fluoroquinazolin-4-yl)-3-methylpiperazine-1-carboxylate

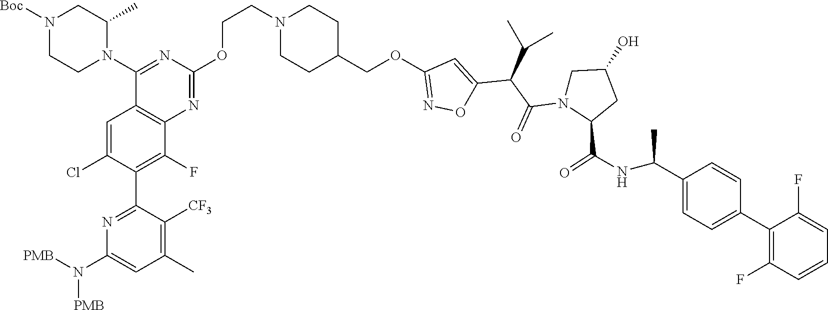

To a solution of (2S,4R)—N—((S)-1-(2′,6′-difluoro-[1,1′-biphenyl]-4-yl)ethyl)-4-hydroxy-1-((R)-3-methyl-2-(3-(piperidin-4-ylmethoxy)isoxazol-5-yl)butanoyl)pyrrolidine-2-carboxamide (276 mg, 0.45 mmol, 1.1 eq) and tert-butyl (3S)-4-(7-(6-(bis(4-methoxybenzyl)amino)-4-methyl-3-(trifluoromethyl) pyridin-2-yl)-6-chloro-8-fluoro-2-(2-oxoethoxy)quinazolin-4-yl)-3-methylpiperazine-1-carboxylate (350 mg, 0.41 mmol, 1 eq) in dichloromethane (5 mL) was added N,N-diisopropylethylamine (159 mg, 1.23 mmol, 3 eq) and sodium triacetoxyborohydride (260 mg, 1.23 mmol, 3 eq). The mixture was stirred at 20° C. for 2 h. LCMS showed the desired mass was detected. The reaction mixture was diluted with water (10 mL) and extracted with dichloromethane (10 mL×3). The combined organic layers were washed with brine (5 mL×3), dried over anhydrous sodium sulfate, filtered and concentrated under reduced pressure to give a residue. The residue was purified by prep-HPLC (column: Phenomenex luna C18 150*25 mm*10 um; mobile phase: [water(FA)-ACN]; B %: 46%-76%, 10 min) to afford the product tert-butyl (3S)-4-(7-(6-(bis(4-methoxybenzyl)amino)-4-methyl-3-(trifluoromethyl)pyridin-2-yl)-6-chloro-2-(2-(4-(((5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy)methyl)piperidin-1-yl)ethoxy)-8-fluoroquinazolin-4-yl)-3-methylpiperazine-1-carboxylate (400 mg, 0.28 mmol, 67% yield) as an off-white solid. LCMS (ESI, m/z): 1447.3 [M+H]⁺.

Step 11 tert-butyl (S)-4-(7-((R)-6-(bis(4-methoxybenzyl)amino)-4-methyl-3-(trifluoromethyl) pyridin-2-yl)-6-chloro-2-(2-(4-(((5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy) methyl)piperidin-1-yl)ethoxy)-8-fluoroquinazolin-4-yl)-3-methylpiperazine-1-carboxylate

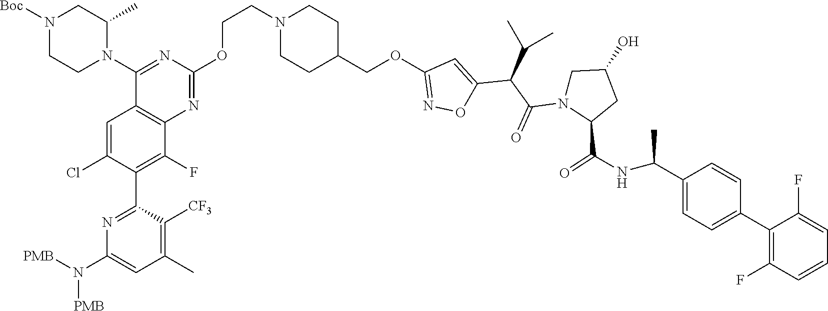

The mixture of atropisomers tert-butyl (3S)-4-(7-(6-(bis(4-methoxybenzyl)amino)-4-methyl-3-(trifluoromethyl)pyridin-2-yl)-6-chloro-2-(2-(4-(((5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy)methyl)piperidin-1-yl) ethoxy)-8-fluoroquinazolin-4-yl)-3-methylpiperazine-1-carboxylate (400 mg, 0.28 mmol, 1.0 eq) was separated by SFC (column: REGIS(S,S)WHELK-O1(250 mm*25 mm, 10 um); mobile phase: [ACN/MeOH(0.1% NH3H2O)]; B %: 55%-55%, 8.0 min), and the second eluent was identified as the desired atropisomer tert-butyl (S)-4-(7-((R)-6-(bis(4-methoxybenzyl)amino)-4-methyl-3-(trifluoromethyl) pyridin-2-yl)-6-chloro-2-(2-(4-(((5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy) methyl)piperidin-1-yl)ethoxy)-8-fluoroquinazolin-4-yl)-3-methylpiperazine-1-carboxylate (170 mg, 0.12 mmol, 42% yield, 99% purity, t_{R}=4.194 min) as a white solid.

Step 12: (2S,4R)-1-((R)-2-(3-((1-(2-((7-((R)-6-amino-4-methyl-3-(trifluoromethyl)pyridin-2-yl)-6-chloro-8-fluoro-4-((S)-2-methylpiperazin-1-yl)quinazolin-2-yl)oxy)ethyl)piperidin-4-yl)methoxy)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide

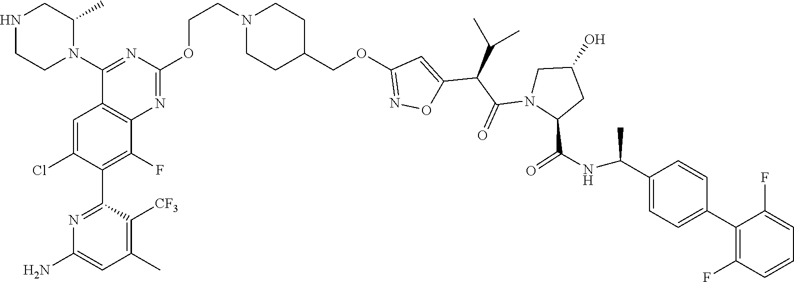

To a solution of tert-butyl (S)-4-(7-((R)-6-(bis(4-methoxybenzyl)amino)-4-methyl-3-(trifluoromethyl)pyridin-2-yl)-6-chloro-2-(2-(4-(((5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)oxy) methyl)piperidin-1-yl)ethoxy)-8-fluoroquinazolin-4-yl)-3-methylpiperazine-1-carboxylate (45 mg, 0.03 mmol, 1 eq) in dichloromethane (2 mL) was added trifluoroacetic acid (0.5 mL) and trifluoromethanesulfonic acid (0.025 mL). The mixture was stirred at 0° C. for 0.5 h. LCMS showed the desired mass was detected. The reaction mixture was concentrated, then quenched by addition saturated aqueous sodium carbonate (2 mL) at 0° C., and extracted with dichloromethane (10 mL×3). The combined organic layers were washed with brine (5 mL×3), dried over anhydrous sodium sulfate, filtered and concentrated under reduced pressure to afford the product (2S,4R)-1-((R)-2-(3-((1-(2-((7-((R)-6-amino-4-methyl-3-(trifluoromethyl)pyridin-2-yl)-6-chloro-8-fluoro-4-((S)-2-methylpiperazin-1-yl)quinazolin-2-yl)oxy)ethyl)piperidin-4-yl)methoxy)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide (30 mg, 0.03 mmol, 87% yield) as a yellow solid. LCMS (ESI, m/z): 1106.6 [M]⁺.

Step 13: (2S,4R)-1-((R)-2-(3-((1-(2-((4-((S)-4-acryloyl-2-methylpiperazin-1-yl)-7-((R)-6-amino-4-methyl-3-(trifluoromethyl)pyridin-2-yl)-6-chloro-8-fluoroquinazolin-2-yl)oxy)ethyl)piperidin-4-yl)methoxy)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide (Example 200)

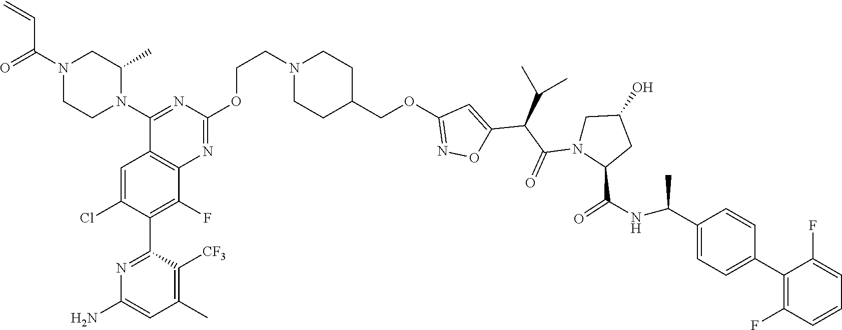

To a solution of (2S,4R)-1-((R)-2-(3-((1-(2-((7-((R)-6-amino-4-methyl-3-(trifluoromethyl)pyridin-2-yl)-6-chloro-8-fluoro-4-((S)-2-methylpiperazin-1-yl)quinazolin-2-yl)oxy)ethyl)piperidin-4-yl)methoxy)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide (30 mg, 0.03 mmol, 1 eq) in dichloromethane (1 mL) was added lutidine (9 mg, 0.09 mmol, 3 eq) and acryloyl chloride (3 mg, 0.03 mmol, 1 eq). The mixture was stirred at −78° C. for 0.5 h. LCMS showed the desired mass was detected. The reaction mixture was diluted with water (5 mL) and extracted with dichloromethane (5 mL×3). The combined organic layers were washed with brine (3 mL×3), dried over anhydrous sodium sulfate, filtered and concentrated under reduced pressure to get a residue. The residue was purified by prep-HPLC (column: Phenomenex luna C18 150*25 mm*10 um; mobile phase: [water(FA)-ACN]; B %: 30%-60%, 10 min) to get the product (2S,4R)-1-((R)-2-(3-((1-(2-((4-((S)-4-acryloyl-2-methylpiperazin-1-yl)-7-((R)-6-amino-4-methyl-3-(trifluoromethyl)pyridin-2-yl)-6-chloro-8-fluoroquinazolin-2-yl)oxy)ethyl)piperidin-4-yl)methoxy)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide (10.69 mg, 0.009 mol, 33% yield, 98% purity) as a white solid. LCMS (ESI, m/z): 1161.4 [M]⁺. ¹H NMR (400 MHz, DMSO-d₆) 8.84-8.39 (m, 1H), 7.81 (s, 1H), 7.51-7.44 (m, 1H), 7.39 (s, 3H), 7.27-7.16 (m, 2H), 6.90-6.81 (m, 2H), 6.49 (s, 1H), 6.24-6.13 (m, 1H), 6.09-5.90 (m, 1H), 5.78-5.70 (m, 1H), 5.19-4.59 (m, 3H), 4.49-4.20 (m, 5H), 4.17-3.92 (m, 4H), 3.72-3.62 (m, 3H), 3.43 (d, J=10.0 Hz, 3H), 3.03-2.92 (m, 3H), 2.69 (s, 2H), 2.37 (d, J=1.2 Hz, 3H), 2.27-2.19 (m, 2H), 2.06-1.98 (m, 3H), 1.85-1.63 (m, 5H), 1.47-1.37 (m, 3H), 1.30-1.24 (m, 4H), 1.00-0.92 (m, 3H), 0.84-0.77 (m, 3H).

Example 201: (2S,4R)-1-((R)-2-(3-(7-(2-(((R)-4-((S)-4-acryloyl-2-methylpiperazin-1-yl)-7-(6-amino-4-methyl-3-(trifluoromethyl)pyridin-2-yl)-6-chloro-8-fluoroquinazolin-2-yl)oxy)ethyl)-2,7-diazaspiro[3.5]nonan-2-yl)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide

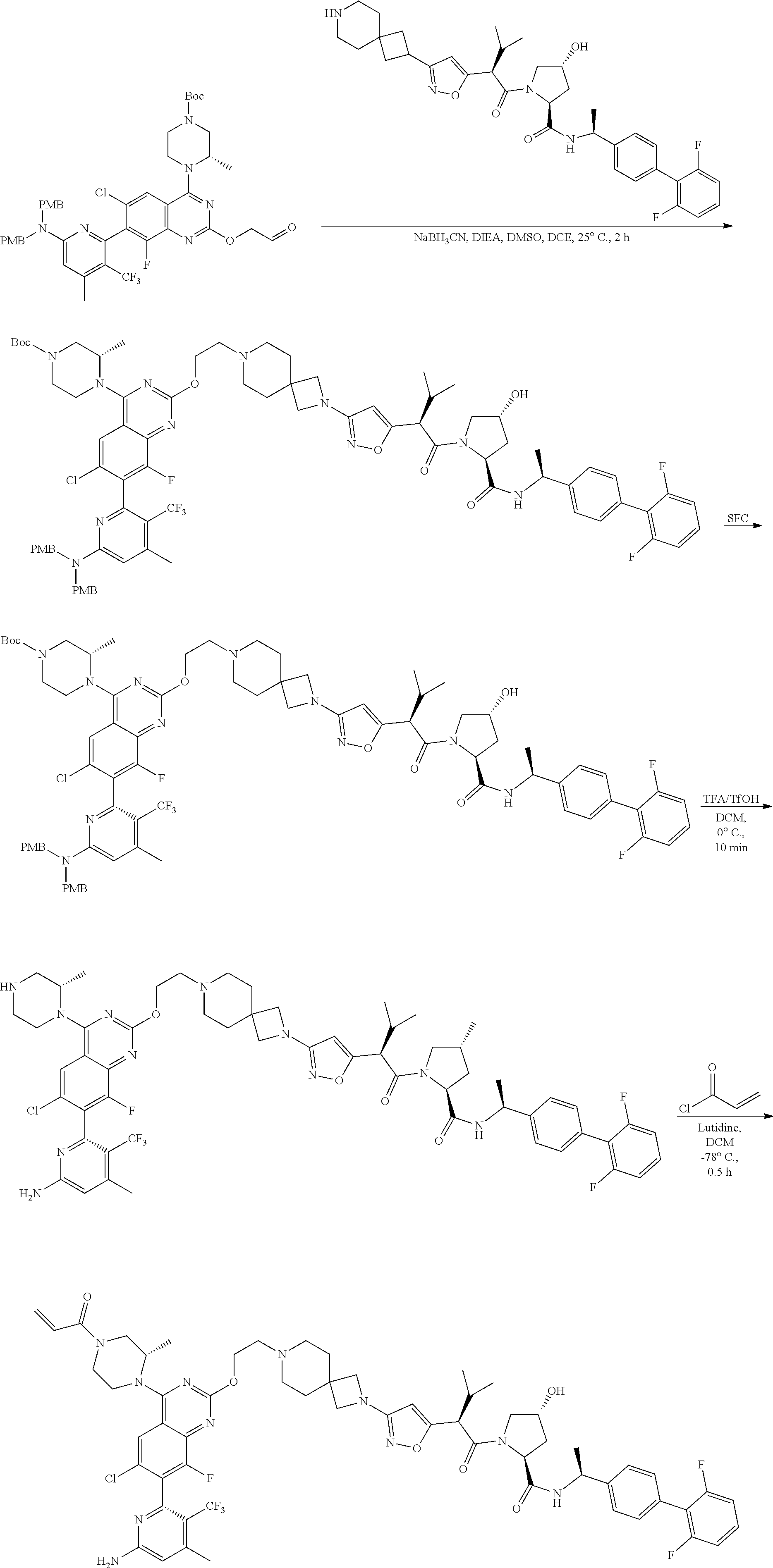

Step 1: tert-butyl (3S)-4-(7-(6-(bis(4-methoxybenzyl)amino)-4-methyl-3-(trifluoromethyl)pyridin-2-yl)-6-chloro-2-(2-(2-(5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)-2,7-diazaspiro[3.5]nonan-7-yl)ethoxy)-8-fluoroquinazolin-4-yl)-3-methylpiperazine-1-carboxylate

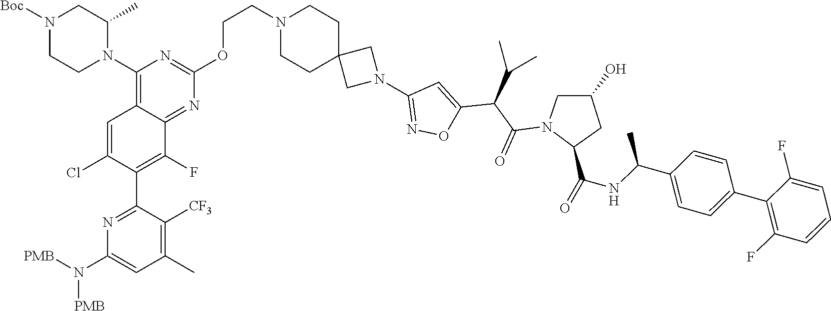

To a solution of tert-butyl (3S)-4-(7-(6-(bis(4-methoxybenzyl)amino)-4-methyl-3-(trifluoromethyl)pyridin-2-yl)-6-chloro-8-fluoro-2-(2-oxoethoxy)quinazolin-4-yl)-3-methylpiperazine-1-carboxylate (460 mg, 0.5 mmol), (2S,4R)-1-((R)-2-(3-(2,7-diazaspiro[3.5]nonan-2-yl)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide (350 mg, 0.5 mmol, trifluoroacetate) in the mixed solvent of dimethylsulfoxide (3 mL) and dichloroethane (3 mL) was added N,N-diisopropylethylamine (168 mg, 1.30 mmol) and sodium triacetoxyborohydride (275 mg, 1.30 mmol). The mixture was stirred at 25° C. for 2 h. The reaction mixture was quenched by adding water (10 mL), and then extracted with ethyl acetate (10 mL×3). The combined organic layers were washed with brine (20 mL×3), dried over sodium sulfate, filtered and concentrated under reduced pressure to give a residue. The residue was purified by prep-HPLC (column: Phenomenex C18 75*30 mm*3 um; mobile phase: [water(FA)-ACN]; B %: 52%-82%, 7 min) to afford the desired product tert-butyl (3S)-4-(7-(6-(bis(4-methoxybenzyl)amino)-4-methyl-3-(trifluoromethyl)pyridin-2-yl)-6-chloro-2-(2-(2-(5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)-2,7-diazaspiro[3.5]nonan-7-yl)ethoxy)-8-fluoroquinazolin-4-yl)-3-methylpiperazine-1-carboxylate (500 mg, 0.34 mmol, 78% yield) as a white solid. LCMS (ESI, m/z): 1458.9 [M]⁺

Step 2: tert-butyl (S)-4-((R)-7-(6-(bis(4-methoxybenzyl)amino)-4-methyl-3-(trifluoromethyl) pyridin-2-yl)-6-chloro-2-(2-(2-(5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)-2,7-diazaspiro[3.5]nonan-7-yl)ethoxy)-8-fluoroquinazolin-4-yl)-3-methylpiperazine-1-carboxylate

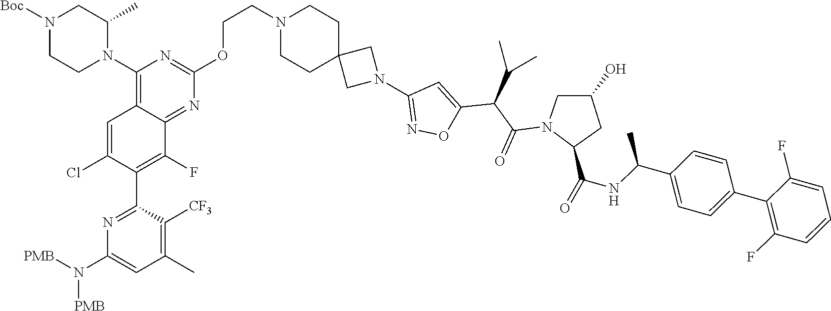

The mixture of atropisomers tert-butyl (3S)-4-(7-(6-(bis(4-methoxybenzyl)amino)-4-methyl-3-(trifluoromethyl)pyridin-2-yl)-6-chloro-2-(2-(2-(5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)-2,7-diazaspiro[3.5]nonan-7-yl)ethoxy)-8-fluoroquinazolin-4-yl)-3-methylpiperazine-1-carboxylate (500 mg, 0.34 mmol) was separated by SFC (column: REGIS(S,S)WHELK-01 (250 mm*25 mm, 10 um); mobile phase:[IPA/ACN]; B %: 55%-55%, 7.2 min). The second eluent was identified as the desired atropisomer tert-butyl (S)-4-((R)-7-(6-(bis(4-methoxybenzyl)amino) -4-methyl-3-(trifluoromethyl) pyridin-2-yl)-6-chloro-2-(2-(2-(5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)-2,7-diazaspiro[3.5]nonan-7-yl)ethoxy)-8-fluoroquinazolin-4-yl)-3-methylpiperazine-1-carboxylate(172 mg, 0.11 mmol, 32% yield, 96% purity, t_{R}=3.062 min) as a yellow solid.

Step 3: (2S,4R)-1-((R)-2-(3-(7-(2-(((R)-7-(6-amino-4-methyl-3-(trifluoromethyl)pyridin-2-yl)-6-chloro-8-fluoro-4-((S)-2-methylpiperazin-1-yl)quinazolin-2-yl)oxy)ethyl)-2,7-diazaspiro[3.5]nonan-2-yl)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide

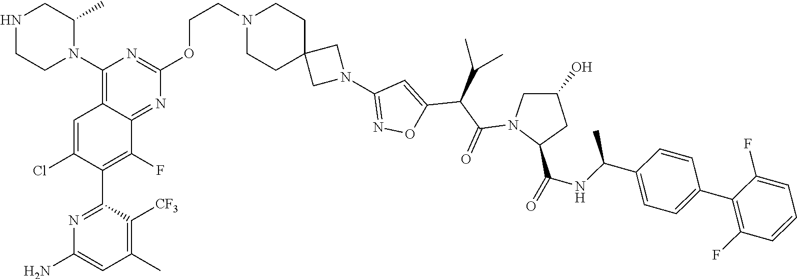

To a solution of tert-butyl (S)-4-((R)-7-(6-(bis(4-methoxybenzyl)amino)-4-methyl-3-(trifluoromethyl)pyridin-2-yl)-6-chloro-2-(2-(2-(5-((R)-1-((2S,4R)-2-(((S)-1-(2′,6′-difluoro-[1,1′-biphenyl]-4-yl)ethyl)carbamoyl)-4-hydroxypyrrolidin-1-yl)-3-methyl-1-oxobutan-2-yl)isoxazol-3-yl)-2,7-diazaspiro[3.5]nonan-7-yl)ethoxy)-8-fluoroquinazolin-4-yl)-3-methylpiperazine-1-carboxylate (100 mg, 0.06 mmol, 1.00 eq) in dichloromethane (2 mL) was added trifluoroacetic acid (1.54 g, 13.51 mmol, 1 mL, 197.06 eq) and trifluoromethanesulfonic acid (1 mL), the mixture was stirred at 0° C. for 10 min. The reaction mixture was concentrated under reduced pressure to give a residue, then the residue was added aqueous sodium carbonate, the mixture was stirred at 25° C. for 10 min. The reaction mixture was diluted with water (10 mL) and extracted with ethyl acetate (10 mL), dried over sodium sulfate, filtered and concentrated under reduced pressure to give the crude product (2S,4R)-1-((R)-2-(3-(7-(2-(((R)-7-(6-amino-4-methyl-3-(trifluoromethyl)pyridin-2-yl)-6-chloro-8-fluoro-4-((S)-2-methylpiperazin-1-yl)quinazolin-2-yl)oxy)ethyl)-2,7-diazaspiro[3.5]nonan-2-yl)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide (76 mg, 0.06 mmol, 99% yield) as a yellow solid. LCMS (ESI, m/z): 1118.6 [M+H]⁺.

Step 4: (2S,4R)-1-((R)-2-(3-(7-(2-(((R)-4-((S)-4-acryloyl-2-methylpiperazin-1-yl)-7-(6-amino-4-methyl-3-(trifluoromethyl)pyridin-2-yl)-6-chloro-8-fluoroquinazolin-2-yl)oxy)ethyl)-2,7-diazaspiro[3.5]nonan-2-yl)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide (Example 201)

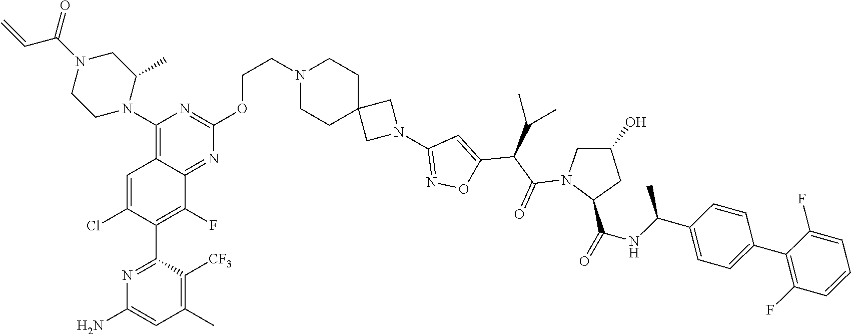

To a solution of (2S,4R)-1-((R)-2-(3-(7-(2-(((R)-7-(6-amino-4-methyl-3-(trifluoromethyl)pyridin-2-yl)-6-chloro-8-fluoro-4-((S)-2-methylpiperazin-1-yl)quinazolin-2-yl)oxy)ethyl)-2,7-diazaspiro [3.5]nonan-2-yl)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide (76 mg, 0.06 mmol, 1.00 eq) in dichloromethane (2 mL) was added 2,6-dimethylpyridine (21 mg, 0.2 mmol, 0.02 mL, 3 eq) at 25° C., then prop-2-enoyl chloride (6 mg, 0.06 mmol, 1.00 eq) was added at −78° C., the mixture was stirred at −78° C. for 0.5 h. The reaction was diluted with water (10 mL) and extracted with dichloromethane (10 mL), dried over sodium sulfate, filtered and concentrated under reduced pressure to give a residue. The residue was purified by prep-HPLC (column: Phenomenex luna C18 150*25 mm*10 um; mobile phase: [water(FA)-ACN]; B %: 29%-59%, 10 min) to afford the desired product (2S,4R)-1-((R)-2-(3-(7-(2-(((R)-4-((S)-4-acryloyl-2-methylpiperazin-1-yl)-7-(6-amino-4-methyl-3-(trifluoromethyl)pyridin-2-yl)-6-chloro-8-fluoroquinazolin-2-yl)oxy)ethyl)-2,7-diazaspiro [3.5]nonan-2-yl)isoxazol-5-yl)-3-methylbutanoyl)-N—((S)-1-(2′,6′-difluoro-[1,1′-biphenyl]-4-yl)ethyl)-4-hydroxypyrrolidine-2-carboxamide (23.38 mg, 0.01 mmol, 29% yield, 100% purity) as a white solid. LCMS (ESI, m/z): 1172.1 [M+H]⁺.

¹H NMR (400 MHz, DMSO-d₆) δ 8.42 (br d, J=7.6 Hz, 1H), 8.24-8.19 (m, 1H), 7.83-7.78 (m, 1H), 7.49-7.44 (m, 1H), 7.39 (s, 4H), 7.25-7.18 (m, 2H), 6.87-6.80 (m, 2H), 6.49 (s, 1H), 6.22-6.14 (m, 1H), 5.83 (s, 1H), 5.76-5.72 (m, 1H), 4.96-4.89 (m, 1H), 4.77-4.64 (m, 1H), 4.43 (br d, J=4.4 Hz, 2H), 4.39-4.33 (m, 1H), 4.33-4.19 (m, 2H), 4.18-4.04 (m, 2H), 4.03-3.90 (m, 1H), 3.72-3.61 (m, 3H), 3.60-3.53 (m, 6H), 3.42-3.39 (m, 1H), 3.39 (br s, 2H), 3.40-3.38 (m, 1H), 2.33 (br d, J=1.6 Hz, 2H), 2.26-2.13 (m, 3H), 2.09-1.98 (m, 2H), 1.83-1.77 (m, 1H), 1.71 (br s, 4H), 1.39 (br d, J=7.2 Hz, 3H), 1.26 (br s, 4H), 0.98-0.91 (m, 3H), 0.84-0.75 (m, 3H), 0.81-0.71 (m, 1H).

### B. Assays & Activity Data

### In-Vitro RAS-RAF Binding Assay (RRB)

Biotinylated KRAS protein amino acids 1-169 (produced at Erasca) is labeled with streptavidin-terbium (lanthanide cryptate donor fluorophore) in assay buffer (50 mM HEPES, pH 7.5, 100 mM NaCl, 1 mM MgCl₂, 1 mM DTT) at a final concentration of 30 nM. In a separate reaction mixture, 30 nM cRAF (RBD) (Abcam, Cambridge MA) is labeled with anti-GST d2 (acceptor fluorophore). Labeling reactions are incubated for 1 hour at room temperature.

Compounds of interest are incubated with the labeled-KRAS for 60 minutes at room temperature at a final DMSO concentration of 5% in a black 1/2 area microtiter plate (50 uL final reaction volume). Following the compound incubation, SOS1 catalytic domain (produced at Erasca) and GTPgS are added to the reaction to initiate nucleotide exchange (60 minutes exchange reaction). Once in the GTP state KRAS will bind to cRAF. No binding will occur if KRAS remains in the GDP state. Compounds may block nucleotide exchange or may create a steric obstruction to cRAF-KRAS interaction by binding to the RAS effector site.

Following the exchange reaction, the labeled KRAS and cRAF are mixed in equal volume (30 uL each) and incubated for 30-60 minutes at room temperature. A portion of this mixture is transferred to a white 384-well plate (20 uL per well in duplicate) and read on an HTRF compatible plate reader (ClarioSTAR). Fluorescent resonance energy transfer (FRET) is measured at equilibrium. FRET signal will be high if KRAS-cRAF binding occurs. FRET signal will be low if KRAS-cRAF binding is inhibited by the test compound. Table 1 and Table 2 below summarizes the results. Table 2 represents the average of multiple runs of each compound.

TABLE 1
Inhibition of RAS-RAF Binding (in-vitro)

| Compound | KRAS (WT) | KRAS (G12D) |
|---|---|---|
| ID | IC₅₀ (nM) | IC₅₀ (nM) |
| 1 | 799 | 15 |
| 2 | 4251 | 17 |
| 3 | >10,000 | 64 |
| 4 | 134 | 13 |
| 5 | 2438 | 19 |
| 6 | 126 | 9.5 |
| 7 | 448 | 12 |
| 8 | 51 | 11 |
| 9 | 49.5 | 13.8 |
| 12 | 86.8 | 11.3 |
| 13 | 49 | 13.6 |
| 14 | 41.9 | 9.4 |

TABLE 2
Inhibition of RAS-RAF Binding (in-vitro)

| Compound | KRAS (WT) | KRAS (G12D) |
|---|---|---|
| ID | IC₅₀ (nM) | IC₅₀ (nM) |
| 1 | 799 | 15.37 |
| 2 | 4251 | 17.18 |
| 3 | >10000 | 64.26 |
| 4 | 216.4 | 15.12 |
| 5 | 4920 | 18.84 |
| 6 | 158.3 | 8.209 |
| 7 | 448.4 | 12.36 |
| 8 | 25.94 | 8.18 |
| 9 | 49.49 | 13.79 |
| 10 | 134.5 | 12.66 |
| 11 | N/A | N/A |
| 12 | 86.83 | 11.25 |
| 13 | 49.11 | 13.58 |
| 14 | 41.87 | 9.397 |
| 15 | 1316 | 12.7 |
| 16 | 939.6 | 9.787 |
| 17 | 2826 | 15.63 |
| 18 | 3140 | 24.88 |
| 19 | 77.73 | 7.668 |
| 20 | 26.91 | 8.521 |
| 21 | 161.9 | 14.44 |
| 22 | | |
| 23 | 64.99 | 12.64 |
| 24 | 75.79 | 8.882 |
| 25 | 107.4 | 11.96 |
| 26 | 91.76 | 11.99 |
| 27 | 37.95 | 10.76 |
| 28 | 24.21 | 7.933 |
| 29 | 27.94 | 9.307 |
| 30 | 22.3 | 7.593 |
| 31 | 27.79 | 8.38 |
| 32 | 30.32 | 8.232 |
| 33 | 3.047 | 7.483 |
| 34 | 63.38 | 9.906 |
| 35 | 196.1 | 12.08 |
| 36 | 1066 | 9.847 |
| 37 | 274.6 | 19.44 |
| 38 | | |
| 39 | | |
| 40 | 26.5 | 8.876 |
| 41 | 53.39 | 9.398 |
| 42 | 24.94 | 7.777 |
| 43 | 38.26 | 7.684 |
| 44 | 34.27 | 8.918 |
| 45 | 44.87 | 8.968 |
| 46 | 21.35 | 5.865 |
| 47 | 22.18 | 7.67 |
| 48 | 24.74 | 7.532 |
| 49 | 24.93 | 7.425 |
| 50 | 98.87 | 10.24 |
| 51 | 95.71 | 9.092 |
| 52 | 113.7 | 7.841 |
| 53 | 123.1 | 8.824 |
| 54 | 52.85 | 9.653 |
| 55 | 67.03 | 9.795 |
| 56 | 65.66 | 9.356 |
| 57 | 117.8 | 13.05 |
| 58 | 65.93 | 9.549 |
| 59 | 73.07 | 7.519 |
| 60 | 188.4 | 9.547 |
| 61 | 1201 | 8.995 |
| 62 | >10000 | 13.38 |
| 63 | 8334 | 10.61 |
| 64 | 1410 | 9.618 |
| 65 | >10000 | 11.1 |
| 66 | 1.00E+04 | 19.75 |
| 67 | 5000 | 20.02 |
| 68 | >10000 | 19.27 |
| 69 | >10000 | 35.15 |
| 70 | 5503 | 28.57 |
| 71 | >10000 | 28.01 |
| 72 | 6727 | 29.43 |
| 73 | >10000 | 47.55 |
| 74 | 962 | 18.15 |
| 75 | 835.6 | 23.88 |
| 76 | 1902 | 21.85 |
| 77 | 1103 | 17.58 |
| 78 | 1673 | 25.5 |
| 79 | >10000 | >10000 |
| 80 | 63.69 | 13.93 |
| 81 | | |
| 82 | 303.9 | 8.523 |
| 83 | 484.1 | 7.698 |
| 84 | 69.03 | 12.8 |
| 85 | | |
| 86 | 568.4 | 9.051 |
| 87 | | |
| 88 | 63.29 | 16.3 |
| 89 | 65.75 | 15.24 |
| 90 | 43 | 13 |
| 91 | 23 | 8.2 |
| 92 | 52 | 8.9 |
| 93 | 54 | 9.1 |
| 94 | 67 | 9.2 |
| 95 | 65 | 8.5 |
| 96 | 156 | 9.2 |
| 97 | 30 | 8.6 |
| 98 | 1434 | 16 |
| 99 | 1605 | 16 |
| 100 | 2074 | 14 |
| 101 | 1794 | 13 |

### Degradation of KRas G12D and Inhibition of KRas G12D-mediated Phosphorylation of ERK1/2 by Exemplary Compounds of Formula (A)

This Example illustrates that the compounds presented herein degrade KRAS G12D.

AsPC-1 cells (ATCC CRL-1682) expressing G12D were grown in RPMI-1640 medium supplemented with 10% fetal bovine serum, and Penicillin/Streptomycin. Cell were plated in 6-well tissue culture plates at a density of 1 million cells/well and allowed to attach for overnight. Diluted compounds were then added in a final concentration of 0.2% DMSO. After 24 hour treatment, the medium was removed, and the plate was washed three times with ice-cold PBS. 50ul of the RIPA cell lysis buffer (ThermoFisher 89901) supplemented with protease inhibitor (Roche 4693116001) and Phosphatase Cocktail A and B inhibitor (Bimake B15002; Bimake Houston TX) was added to each well, the adherent cells were scratched off, and transferred into pre-cold centrifuge tubes. After vigorous vortex, the tubes were incubated on ice for 30 minutes, centrifuged 14,000 rpm for 10 min at 4° C., and the supernatant were transferred into fresh tubes kept on ice. The protein concentration of the supernatant was determined with bicinchoninic acid method (BCA) (ThermoFisher 23227). Samples were prepared to the final concentration of 30ug in 20ul using NuPAGE LDS Sample Buffer (Invitrogen NP0007), NuPAGE Sample Reducing Agent (Invitrogen NP0009), and RIPA cell lysis buffer, and boiled at 95° C. for 15 minutes in water bath.

The levels of KRAS protein and phosphorylated ERK1/2 were determined by Western blotting. 20 μL of protein sample mixture was loaded into each well of 1.5 mm 4-12% Bis-Tris gel (Invitrogen NP0335), and run at 80V in stacking gel and 120 V in separating gel for about 2 h in MOPS running buffer (Invitrogen NP0001). The gel was transferred to the membrane (20V, 7 min) with Invitrogen iBlot2, and blocked with Li-Cor blocking buffer (LI-COR 927-60001; Li-Cor Biotechnology, Lincoln NE) for at least 1 h at room temperature. Primary antibodies diluted in the blocking buffer were added and incubated at 4° C. overnight. As the primary antibodies, KRAS (Proteintech 12063-1-AP), G12D Mutant Specific KRAS (Cell Signaling Technology 14429S), ERKI1/2 (Cell Signaling Technology 4695S), Phospho-ERKI1/2 (Cell Signaling Technology 4370S), and GAPDH (Millipore MAB374) were used. The membrane was washed 4 times with TBST (0.10% Tween20®; ThermoFisher 28360) for 5 minutes at room temperature, then incubated with Li-Cor anti-rabbit-800 (LI-COR 926-68070) and anti-mouse-680 (LI-COR-926-32211) diluted in Li-Cor blocking buffer with the ratio of 1:15,000 to visualize the primary antibodies for 1 hour at room temperature. Then membrane was washed 4 times with TBST (0.1% Tween20) 5 minutes. The signal intensities of bands were quantified by Li-Cor Odyssey CLX.

The KRas and phosphor-ERK1/2 signals were normalized to the GAPDH signal for each lane and percent of DMSO values were calculated to determine the degradation of KRas and the inhibition of ERK1/2 phosphorylation upon the compound treatment.

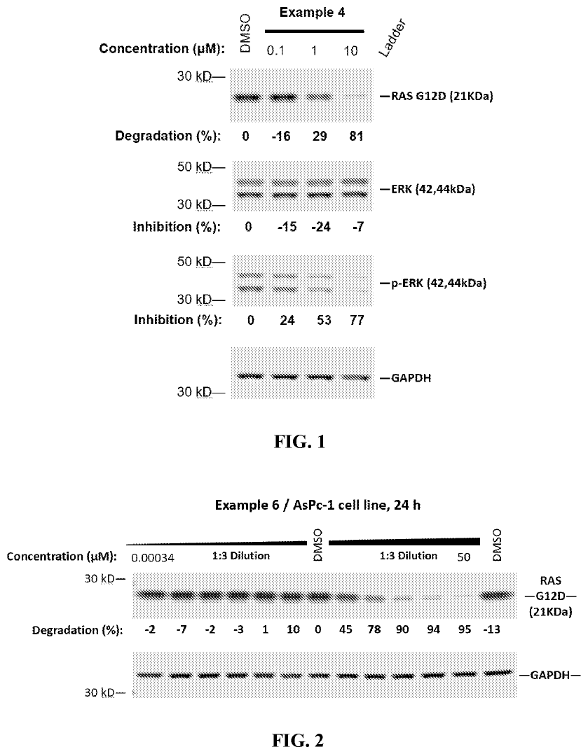

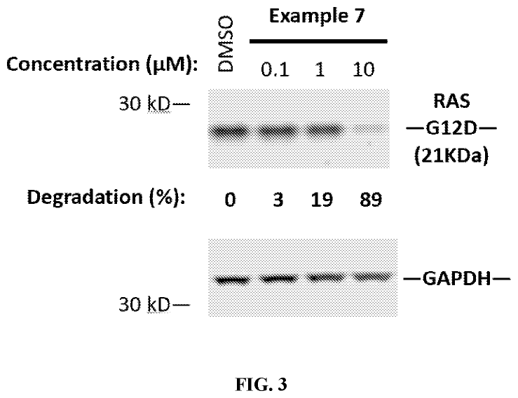

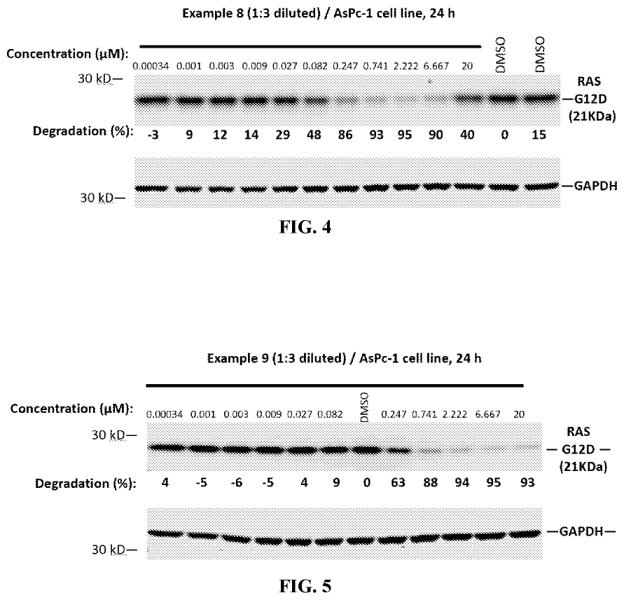

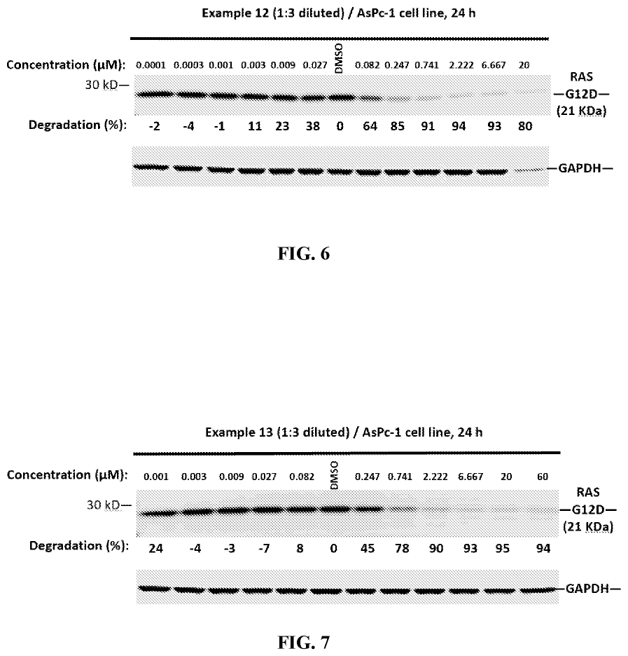

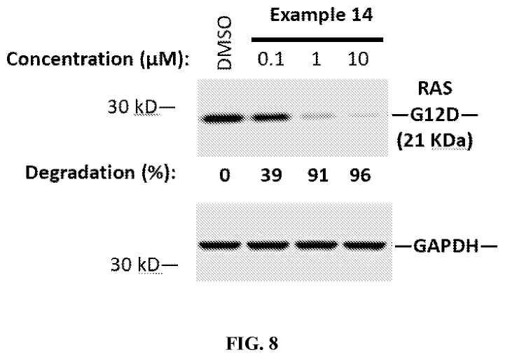

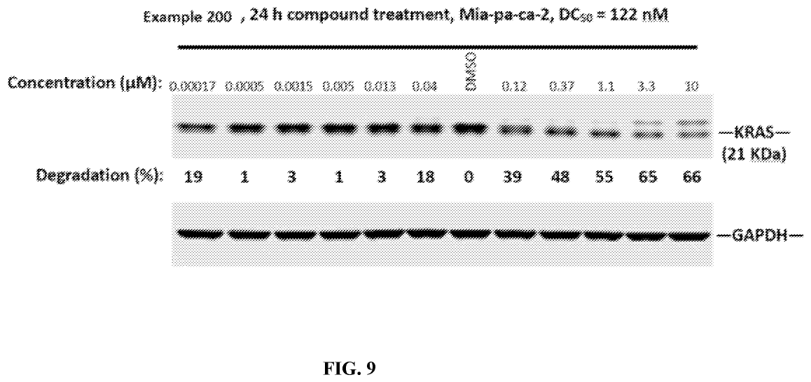

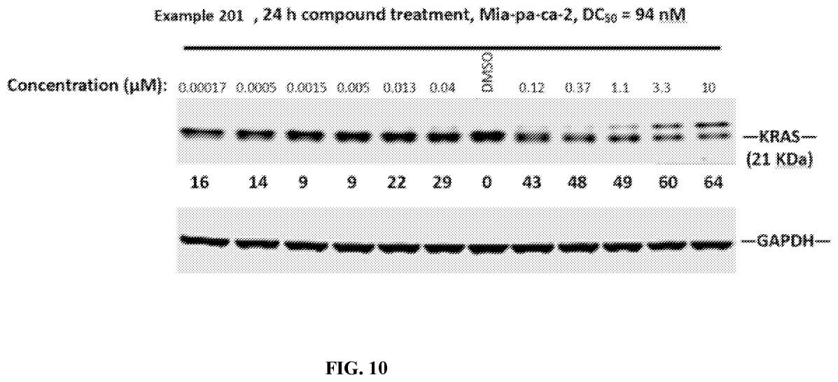

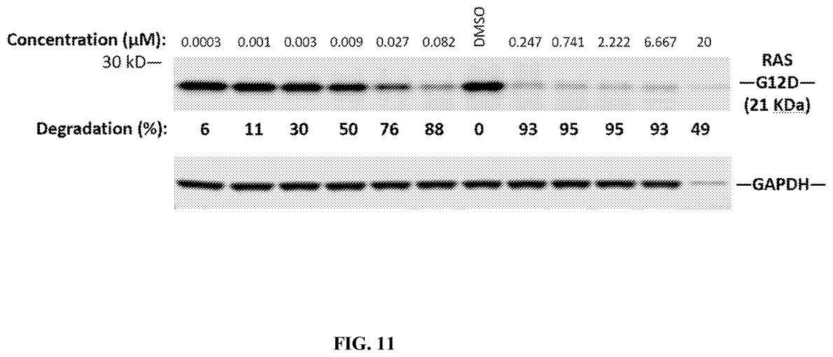

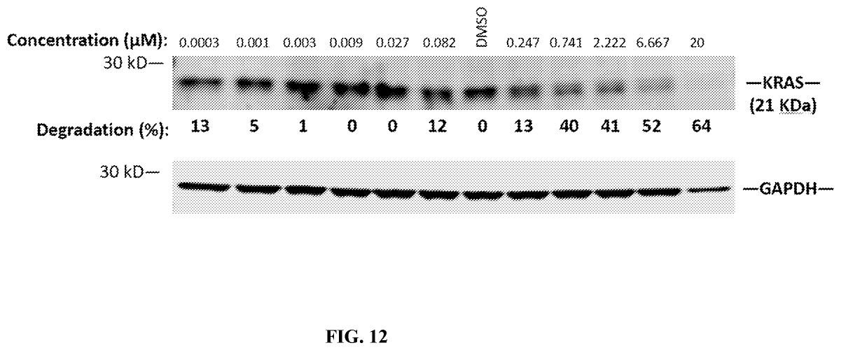

The results for the conjugate of Example 4 is illustrated in FIG. 1. The blot indicates successful degradation of KRAS G12D. Favorable results were also obtained for KRAS G12D degradation in Example 6, Example 7, Example 8, and Example 9, as illustrated in FIGS. 2-5, respectively.

### ASPC-1 HiBit Assay to Determine G12D Degradation

Compounds presented herein induce degradation of the KRas G12D (KRasG12D) upon treatment of engineered cancer cell line.

The degradation of the KRasG12D in cellular environment was measured using engineered homozygous KRasG12D AsPC-1 cell line in which KRas G12D was fused to luminescent HiBiT peptide at the N-terminal. The small (1.3 kDa) HiBiT peptide complements with high affinity to a larger (18 kDa) subunit evolved from NanoLuc (termed LgBiT). The resulting complex (i.e., reconstituted luciferase enzyme) generates bright luminescence that translates to high sensitivity (1 amol), broad dynamic range (four orders of magnitude), and rapid kinetics for real time quantitation.

To generate HiBit KRasG12D knock-in cell line, sgRNA (SEQ. ID NO: 1) and ssODN (SEQ. ID NO: 2) were designed and synthesized at GenScript.

RNP complex was generated by mixing of 100 pmol of Cas9 protein to PCR tubes containing 300 pmol sgRNA incubating the resultant complex at room temperature for 20 min. 2 pg ssODN was added to RNP complex slowly and RNP-ssODN complex was incubated at room temperature for 10 min. RNP-ssODN complex was mixed with 100 ml of AsPC-1 cells (1×10⁶ cells total) and electroporated using Nucleofactor (Program CM-150). Cells were then incubated at ambient temperature for 5 min and transferred to a six-well plate containing 2 ml growth medium.

To select pools with the highest luminescence signal, 10⁴ cells of parental AsPC-1 and transfected pools were plated in solid white 384-well tissue culture plates. 24 hours after seeding, HiBiT was detected using the Nano-Glo HiBiT Lytic Detection System (Promega) following manufacturer's instructions. Briefly, 500 μl of Nano-Glo HiBiT Lytic Detection Reagent was added directly to the cells and mixed by inversion before recording luminescence on an Envision (Perkin-Elmer) with 0.5 s integration time.

Single clones were isolated from stable pool by limited dilution method and presence of HiBiT knock-in was verified by TA cloning and subsequent sequencing. Additionally, western blotting analysis was conducted to confirm selection of single clones with high luminescence signal.

For DC_{50S} measurements, AsPC-1 HiBiT single clone cells were grown in RPMI 1640 medium (Invitrogen 22400-089) supplemented with 10% fetal bovine serum. Cells were seeded in 384-well tissue culture plates at a density of 7,000 cells/well and allowed to attach for 24 hours.

Test compounds were distributed directly into assay plate with the adjustment for DMSO concentration using Tecan HP D300e. For most of the experiments, the final concentration of compounds starts from 10 μM, 3-fold dilution dose response and 10 doses. In those cases where compound hook effect was observed at 10 μM or compound potency was left shifted, the top dose was adjusted to 2.5 mM. DMSO final concentration is 1%. Each concentration of exemplary compound was tested as singleton. The negative control wells were cells with DMSO only, and the positive control wells were cells with 10 mM inhibitor.

Following 24-hour incubation with the compounds plate and Nano-Glo® HiBiT Lytic reagents were equilibrated to room temperature. 20 μL of HiBiT Lytic reagent were added to each well. The assay plate was incubated on digital microplate shaker (350 rpm) for 20 min followed by centrifugation at 500 g for 1 min. Luminescence was recorded using Envision (Perkin-Elmer) using aperture 384-L1 with 0.1 s integration time.

HiBiT signal are used to calculate the % degradation at given compound concentration using the following formula:

% Degradation=(1−(Sample HiBiT signal−Ave Min signal)/(Ave Max signal−Ave Min signal))*100%

For DC₅₀ calculation, the data with the hook effect were removed first and DC_{50S} was reported using a 4-parameter fit of dose response curve using GraphPad in those cases where maximum % degradation was >50% and Minimal % degradation was <50%. The decrease in DC₅₀ reflects that the exemplary compound led to a higher level of degradation of KRasG12D than another exemplary compound at specific timepoint of treatment of cancer cell line.

For percent degradation measurements, AsPC-1 HiBiT single clone cells were grown in RPMI 1640 medium (Invitrogen 22400-089) supplemented with 10% fetal bovine serum. Cells were seeded in 384-well tissue culture plates at a density of 7,000 cells/well and allowed to attach for 24 hours.

Following 24-hour incubation with the compounds, plate and Nano-Glo® HiBiT Lytic reagents were equilibrated to room temperature. 20 μL of HiBiT Lytic reagent were added to each well. The assay plate was incubated on digital microplate shaker (350 rpm) for 20 min followed by centrifugation at 500 g for 1 min. Luminescence was recorded using Envision (Perkin-Elmer) using aperture 384-L1 with 0.1 s integration time.

HiBiT signal are used to calculate the % degradation at 1 mM compound PGP502,E concentration using the following formula:

$$\% Degradation = ( 1 - ( Sample HiBiT signal - Ave Min signal ) / Ave Max signal - Ave Min signal ) ) * 100 \%$$

The decrease in % degradation reflects that the exemplary compound led to a higher level of degradation of KRasG12D than another exemplary compound at specific timepoint of treatment of cancer cell line.

TABLE 3
G12D degradation in ASPC-1 cell line.

| | ASPC-1 HiBit | ASPC-1 |
|---|---|---|
| | % degradation | HiBit DC50 |
| Ex.# | at 1 μM | (C), nM |
| 1 | 29.32 | |
| 2 | −0.69 | |
| 3 | −5.46 | |
| 4 | 51.32 | |
| 5 | −22.88 | |
| 6 | 79.35 | |
| 7 | 38.07 | |
| 8 | 81.44 | 126 |
| 9 | 74.79 | |
| 10 | −20.40 | |
| 11 | N/A | |
| 12 | 85.28 | 77.5 |
| 13 | 63.17 | 443.7 |
| 14 | 76.19 | 331.2 |
| 15 | 78.41 | 112.1 |
| 16 | 82.06 | 69.7 |
| 17 | 81.56 | 244.3 |
| 18 | 82.39 | 247.1 |
| 19 | 86.59 | 63.2 |
| 20 | 86.42 | 22.7 |
| 21 | ND | |
| 22 | 74.77 | |
| 23 | 71.14 | |
| 24 | 39.65 | |
| 25 | 84.30 | |
| 26 | 79.18 | |
| 27 | 59.16 | |
| 28 | 17.77 | |
| 29 | 46.39 | |
| 30 | 24.89 | |
| 31 | 37.68 | |
| 32 | 61.66 | |
| 33 | 11.41 | |
| 34 | 72.86 | |
| 35 | 70.63 | 467.5 |
| 36 | 77.04 | |
| 37 | 82.11 | |
| 38 | ND | |
| 39 | ND | |
| 40 | 61.06 | |
| 41 | 25.83 | |
| 42 | 78.29 | |
| 43 | 50.69 | |
| 44 | 78.02 | |
| 45 | 70.94 | |
| 46 | −42.07 | |
| 47 | −17.75 | |
| 48 | −12.47 | |
| 49 | 76.63 | 323.5 |
| 50 | −11.00 | |
| 51 | 74.80 | |
| 52 | 68.81 | |
| 53 | 73.22 | |
| 54 | 77.67 | |
| 55 | 77.06 | |
| 56 | 23.46 | |
| 57 | 41.46 | |
| 58 | 76.57 | |
| 59 | 69.84 | |
| 60 | 84.62 | 108 |
| 61 | 82.52 | |
| 62 | 83.57 | |
| 63 | 79.80 | |
| 64 | 79.40 | |
| 65 | 79.15 | |
| 66 | 81.14 | |
| 67 | −10.03 | 10000 |
| 68 | 60.49 | 1284 |
| 69 | 75.39 | 525 |
| 70 | 76.97 | 500.6 |
| 71 | 76.34 | 581.5 |
| 72 | 74.62 | 528.4 |
| 73 | 74.50 | 348.7 |
| 74 | 81.27 | 184.6 |
| 75 | 67.95 | 843.4 |
| 76 | 79.20 | 258.6 |
| 77 | 77.53 | 329.7 |
| 78 | 78.29 | 294.4 |
| 79 | 34.43 | |
| 80 | ND | |
| 81 | ND | |
| 82 | 42.12 | |
| 83 | 6.25 | |
| 84 | 74.63 | |
| 85 | 28.75 | |
| 86 | 11.41 | |
| 87 | −15.10 | |
| 88 | 73.19 | |
| 89 | ND | |
| 90 | ND | |
| 91 | | 23 nM |
| 92 | | 49 nM |
| 93 | | 34 nM |
| 94 | | 38 nM |
| 95 | | 43 nM |
| 96 | | 75 nM |
| 97 | | 135 nM |
| 98 | | 107 nM |
| 99 | | 264 nM |
| 100 | | 197 nM |
| 101 | | 707 nM |

### Degradation of KRas G12C

MIA PaCa-2 cells (ATCC CRL-1420) expressing G12C were grown in DMEM medium supplemented with 10% fetal bovine serum, 2.5% horse serum, 10 ug/ml blasticidin (InvivoGen ant-bl-1) and Penicillin/Streptomycin. Cells were plated in 6-well tissue culture plates at a density of 1 million cells/well and allowed to attach for overnight. Diluted compounds were then added in a final concentration of 0.2% DMSO. After 24-hour treatment, the medium was removed, and the plate was washed three times with ice-cold PBS. 50ul of the RIPA cell lysis buffer (ThermoFisher 89901) supplemented with protease inhibitor (Roche 4693116001) and Phosphatase Cocktail A and B inhibitor (Bimake B15002; Bimake Houston TX) was added to each well, the adherent cells were scratched off, and transferred into pre-cold centrifuge tubes. After vigorous vortex, the tubes were incubated on ice for 30 minutes, centrifuged 14,000 rpm for 10 min at 4° C., and the supernatant were transferred into fresh tubes kept on ice. The protein concentration of the supernatant was determined with bicinchoninic acid method (BCA) (ThermoFisher 23227). Samples were prepared to the final concentration of 30ug in 20ul using NuPAGE LDS Sample Buffer (Invitrogen NP0007), NuPAGE Sample Reducing Agent (Invitrogen NP0009), and RIPA cell lysis buffer, and boiled at 95° C. for 15 minutes in water bath.

The levels of KRas protein was determined by Western blotting. 20 μL of protein sample mixture was loaded into each well of 1.5 mm 4-12% Bis-Tris gel (Invitrogen NP0335), and run at 80V in stacking gel and 120 V in separating gel for about 2 h in MOPS running buffer (Invitrogen NP0001). The gel was transferred to the membrane (20V, 7 min) with Invitrogen iBlot2, and blocked with Li-Cor blocking buffer (LI-COR 927-60001; Li-Cor Biotechnology, Lincoln NE) for at least 1 h at room temperature. Primary antibodies diluted in the blocking buffer were added and incubated at 4° C. overnight. As the primary antibodies, KRAS (Proteintech 12063-1-AP), and GAPDH (Millipore MAB374) were used. The membrane was washed 4 times with TBST (0.10% Tween20; ThermoFisher 28360) for 5 minutes at room temperature, then incubated with Li-Cor anti-rabbit-800 (LI-COR 926-68070) and anti-mouse-680 (LI-COR-926-32211) diluted in Li-Cor blocking buffer with the ratio of 1:15,000 to visualize the primary antibodies for 1 hour at room temperature. Then membrane was washed 4 times with TBST (0.1% Tween20) 5 minutes. The signal intensities of bands were quantified by Li-Cor Odyssey CLX.

The KRas signals were normalized to the GAPDH signal for each lane and percent of DMSO values were calculated to determine the degradation of KRas upon the compound treatment.

Degradation of KRAS wild type (WT) by Exemplary Compounds of Formula (A)

This Example illustrates that embodiments of the present application degrade KRAS WT

HT-29 cells (ATCC HTB-38) expressing WT KRAS were grown in McCoy's 5a medium modified (ATCC cat #30-2007) supplemented with 10% fetal bovine serum, and Penicillin/Streptomycin. Cell were plated in 6-well tissue culture plates at a density of 1 million cells/well and allowed to attach for overnight. Diluted compounds were then added in a final concentration of 0.2% DMSO. After 24 hour treatment, the medium was removed, and the plate was washed three times with ice-cold PBS. 50ul of the RIPA cell lysis buffer (ThermoFisher 89901) supplemented with protease inhibitor (Roche 4693116001) and Phosphatase Cocktail A and B inhibitor (Bimake B15002; Bimake Houston TX) was added to each well, the adherent cells were scratched off, and transferred into pre-cold centrifuge tubes. After vigorous vortex, the tubes were incubated on ice for 30 minutes, centrifuged 14,000 rpm for 10 min at 4° C., and the supernatant were transferred into fresh tubes kept on ice. The protein concentration of the supernatant was determined with bicinchoninic acid method (BCA) (ThermoFisher 23227). Samples were prepared to the final concentration of 30ug in 20ul using NuPAGE LDS Sample Buffer (Invitrogen NP0007), NuPAGE Sample Reducing Agent (Invitrogen NP0009), and RIPA cell lysis buffer, and boiled at 95° C. for 15 minutes in water bath.

The levels of KRAS protein were determined by Western blotting. 20 μL of protein sample mixture was loaded into each well of 1.5 mm 4-12% Bis-Tris gel (Invitrogen NP0335), and run at 80V in stacking gel and 120 V in separating gel for about 2 h in MOPS running buffer (Invitrogen NP0001). The gel was transferred to the membrane (20V, 7 min) with Invitrogen iBlot2, and blocked with Li-Cor blocking buffer (LI-COR 927-60001; Li-Cor Biotechnology, Lincoln NE) for at least 1 h at room temperature. Primary antibodies diluted in the blocking buffer were added and incubated at 4° C. overnight. As the primary antibodies, KRAS (Proteintech 12063-1-AP), and GAPDH (Millipore MAB374) were used. The membrane was washed 4 times with TBST (0.10% Tween20; ThermoFisher 28360) for 5 minutes at room temperature, then incubated with Li-Cor anti-rabbit-800 (LI-COR 926-68070) and anti-mouse-680 (LI-COR-926-32211) diluted in Li-Cor blocking buffer with the ratio of 1:15,000 to visualize the primary antibodies for 1 hour at room temperature. Then membrane was washed 4 times with TBST (0.1% Tween20) 5 minutes. The signal intensities of bands were quantified by Li-Cor Odyssey CLX.

The KRAS signals were normalized to the GAPDH signal for each lane and percent of DMSO values were calculated to determine the degradation of KRAS upon the compound treatment.

### CAF Assay

This Example provides a protocol for assessing covalent adduct formation (CAF) between the compounds provided herein and KRAS.

In vitro covalent adduct formation assay: Covalent adduct formation (CAF) reactions between Cys12 of the KRAS 4B G12C protein and embodiments of the application were measured in vitro using liquid chromatography-mass spectrometry (LC-MS).

Recombinant Human KRAS 4B protein containing the G12C mutation was used in compound screening experiments. This protein contained 188 amino acids in total, including an N-terminal 6-Histidine tag, followed by a Tobacco Etch Virus (TEV) tag, followed by residues 1-169 of the native KRAS 4B sequence. The exact mass of the protein was 21,310 Da as determined by mass spectrometry. The full sequence can be found in International Application No. PCT/US2020/065966, the sequence for which is incorporated by reference herein.

In an alternative screen, the assay can be conducted using a KRAS 4b G12C protein having 170 amino acids, a mass of 19,336 Da, and an amino sequence also found in International Application No. PCT/US2020/065966, the sequence for which is incorporated by reference herein.

The recombinant protein was expressed in E. coli BL21 cells and purified using affinity chromatography via a Ni-NTA column. Protein stocks were nucleotide-exchanged to >95% GDP, concentrated to 4 mg/mL, and stored at −80° C. in storage buffer (50 mM HEPES pH 7.4, 50 mM NaCl, 5 mM MgCl2, 1 mM DTT). Pure KRAS 4B G12C protein was diluted to a concentration of 5 μM in Tris Buffered Saline, pH 7.4. The compounds were dissolved in DMSO and added to the diluted protein to make a 10 μM concentration. The total DMSO concentration in the reaction was 4%. The reaction was mixed by pipetting and incubated at 22° C. for one hour. Aliquots of the reaction were taken over time and diluted 2:1 in 0.10% formic acid. The intact mass of the protein samples was measured by LC-MS using a QExactive+mass spectrometer (Thermo Scientific). An amount of 500 ng total protein was injected onto a C8 reverse phase column, eluted with a seven-minute gradient of 30%-90% acetonitrile/0.10% formic acid, and analyzed for intact mass by the mass spectrometer. Adducts identified were confirmed to be within 1 Dalton of the expected mass, and the relative ratios of free:adduct protein were used to quantify the percentage of protein bound by the compound. CAF reactions were run in duplicate, with a typical variability of ±5%.

### CAF Assay Results

| Example # | M + H | % CAF at 5 min |
|---|---|---|
| 200 | 1161.4549 Da | 97% |
| 201 | 1172.4708 Da | 98% |

Although the foregoing embodiments have been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

INFORMAL SEQUENCE LISTING

| SEQ | | |
|---|---|---|
| ID NO: | Name of sequence | Sequence |
| 1 | sgRNA | AATGACTGAATATAAACTTG |
| 2 | Single-stranded | ATTAACCTTATGTGTGACATGTTCTA |
| | donor | ATATAGTCACATTTTCATTATTTTTA |
| | oligonucleotides | TTATAAGGCGTGCTGAAAATGgtgag |
| | (ssODN) | cggctggcggctgttcaagaagatta |
| | | gcACTGAATATAAACTTGTAGTCGTA |
| | | GGTGCTGATGGCGTAGGCAAGAGTGC |
| | | CTTGACGATACAGCTAATTCAGAATC |
| | | ATTTTGTGGACGAATA |

What is claimed is:

1. A conjugate of Formula (A): 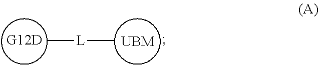
wherein G12D is a KRAS inhibitor capable of binding to a KRAS protein having a G12D mutation; L is a bivalent linker that connects G12D to a ubiquitin binding moiety (UBM); and wherein UBM binds to a ubiquitin ligase.

2. The conjugate of claim 1, wherein the conjugate is a structure of Formula (I): 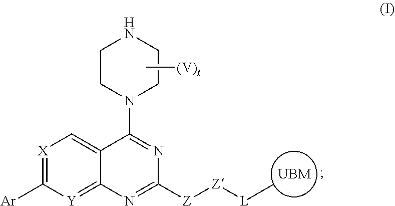
wherein t is integer from 0 to 4;
Ar is selected from 6- to 10-membered aryl, 5-10 membered heteroaryl;
wherein the 5-10 membered heteroaryl ring contains 1 to 4 heteroatoms independently selected from O, N and S; and wherein Ar is optionally substituted with 1 to 5 substituents independently selected from the group consisting of OH, Oxo (═O), halo, CN, NR′R″, C₁₋₄ alkyl, CF₃, C₁₋₄ haloalkyl, C₁₋₄ alkoxy, C₃₋₄ cycloalkyl, C₂₋₄ alkynyl; wherein R′ and R″ are independently selected from alkyl and hydrogen;
G is O or S;
each V is independently selected from methyl, cyanomethyl, or any two V combine to form a bridge or spirocycle structure optionally comprising a heteroatom in the bridge or spirocycle selected from S, SO₂, O or N, and wherein the bridge or spirocycle structure is optionally substituted with oxo;
X is C—H, C-halo, C—C₁₋₃ alkyl, CF₃, C—C₁₋₃ haloalkyl, C—C₃₋₄ cycloalkyl, C-cyano, N;
Y is C—H, C-halo, C—C₁₋₃ alkyl, C—C₃₋₄ cycloalkyl, C-cyano, N;
Z is O, NR^{Z}, S, or absent; wherein R^{Z} is hydrogen or methyl;
Z′ is null, substituted or unsubstituted alkylene, substituted or unsubstituted heterocyclylalkylene, substituted or unsubstituted heterocyclyloxyalkylene, substituted or unsubstituted alkoxalkylene, substituted or unsubstituted heteroalkylene, substituted or unsubstituted arylalkylene, substituted or unsubstituted aryloxyalkylene, substituted or unsubstituted heteroarylalkylene, substituted or unsubstituted heteroaryloxyalkylene, substituted or unsubstituted cycloalkylalkylene, or a substituted or unsubstituted cycloalkyloxyalkylene; and
L is: bond, NH, S, O, C(O), C(O)O, OC(O), NHC(O), C(O)NH, NHC(O)NH, NHC(NH)NH, C(S), substituted or unsubstituted alkylene, substituted or unsubstituted heteroalkylene, substituted or unsubstituted cycloalkylene, substituted or unsubstituted spirocycloalkylene, substituted or unsubstituted heterocycloalkylene, substituted or unsubstituted spiroheterocycloalkylene, substituted or unsubstituted arylene, substituted or unsubstituted heteroarylene or combinations thereof.

3. The conjugate of claim 2, wherein Ar is selected from: 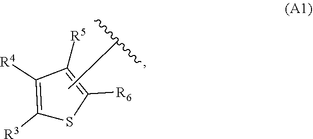
wherein R³, R⁴, R⁵, and R⁶ are independently selected from halogen, hydrogen, hydroxyl, alkoxy, alkyl, cycloalkyl, amino, N-alkylamino, C-amide (—CONRR′), N-amides (—NHCOR), urea (—NHCONHR), ether (—OR), sulfonamide (—NHSO₂R or —SO₂NHR), and CF₃; wherein each R and R′ is independently hydrogen, alkyl, or cycloalkyl; or any two adjacent R³, R⁴, R⁵, or R⁶ form an optionally substituted fused 5- or 6-membered ring comprising 0 to 3 heteroatoms selected from N, O or S; provided that one of R³, R⁴, R⁵, or R⁶ is the bond representing the link between A and the tricyclic ring system; 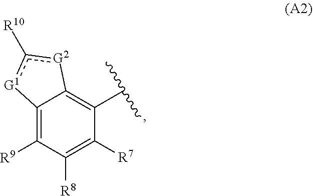
wherein G¹ and G² are independently selected from S, O, CH, —CH═CH—, —C-E, —CR²⁰═CHR²¹—, wherein R²⁰ and R²¹ is hydrogen, halogen, alkyl or haloalkyl, and at least one of R²⁰ and R²¹ is halogen, —CH═N—, N, NH, NMe or —CE, where E is CN, halogen, OH, OMe, alkyl- or aryl sulfonamide, alkyl- or aryl sulfone, acyl, formyl, amide, ester, carboxylic acid, or CF₃; wherein either G¹ or G² forms a double bond with carbon of the —CR¹⁰ moiety; wherein R⁷, R⁸, and R⁹, are independently selected from halogen, hydrogen, hydroxyl, alkoxy, alkyl, alkynyl, cycloalkyl, amino, N-alkylamino; and R¹⁰ is hydrogen, hydroxyl, halogen, alkoxy, amino, N-alkylamino, C-amide (—CONRR′), N-amides (—NHCOR), urea (—NHCONHR), ether (—OR), or sulfonamide (—NHSO₂R or —SO₂NHR)₃; 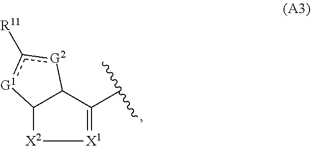
wherein G¹ and G² are independently selected from S, O, CH, —CH═CH—, —CH═N—, N, NH, NMe or —CE, where E is CN, halogen, OH, OMe, alkyl- or aryl sulfonamide, alkyl- or aryl sulfone, acyl, formyl, amide, ester, carboxylic acid, or CF₃; wherein either G¹ or G² forms a double bond with carbon of the —CR¹¹ moiety; R¹¹ is hydroxyl, alkoxy, amino, N-alkylamino, C-amide (—CONRR′), N-amides (—NHCOR), urea (—NHCONHR), ether (—OR), or sulfonamide (—NHSO₂R or —SO₂NHR)₃, wherein R and R′ are independently alkyl or hydrogen; wherein X¹ is selected from CH, or N; and X² is selected from O, S, NH, and NMe; 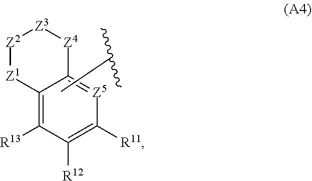
wherein Z¹, Z², Z³, and Z⁴ are independently selected from N, NH, CH, C-halo, C-alkynyl, C═O, C═S, point of attachment: 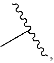
S, or null; Z⁵ is CH, C-halo, or point of attachment: 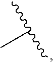
wherein null can only occur once, and at least one of Z¹, Z², Z³, and Z⁴ is NH and at least one of Z¹, Z², Z³, and Z⁴ adjacent to the NH is C═O or C═S; and wherein R¹¹, R¹², and R¹³, are independently selected from halogen, hydrogen, hydroxyl, alkoxy, alkyl, cycloalkyl, amino, N-alkylamino; or 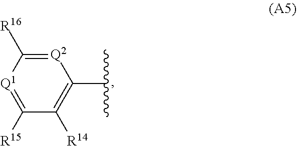
wherein Q¹ and Q² are independently selected from N, —CH, C-halogen, with the proviso that at least one of Q¹ and Q2 is N; R¹⁴ and R¹⁵ are selected from halogen, hydrogen, hydroxyl, alkoxy, alkyl, CF₃, haloalkyl, cycloalkyl, amino, and N-alkylamino; or R¹⁴ and R¹⁵ combine o form a fused aryl or heteroaryl ring optionally substituted with one or more alkyl, halogen, or haloalkyl; and R¹⁶ is hydroxyl, alkoxy, amino, N-alkylamino, C-amide (—CONRR′), N-amide (—NHCOR), urea (—NHCONHR), ether (—OR), or sulfonamide (—NHSO₂R or —SO₂NHR)₃, wherein R and R′ each nitrogen in structures A1 to A5 are independently alkyl or hydrogen.

4. The conjugate of claim 2 or 3, wherein G is 0.

5. The conjugate of claim 2 or 3, wherein G is S.

6. The conjugate of any one of claims 3 to 5, wherein (A1) is selected from: 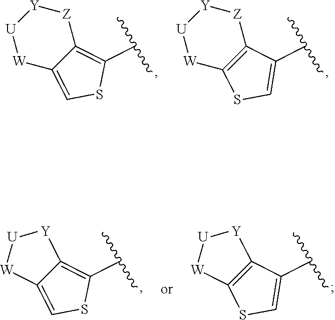
wherein each W, U, Y, and Z are independently selected from C═O, NH, O, S, N, CH, C-Q, where Q is amino, alkylamino, OH, halogen, methyl, —O-alkyl, —O— cycloalkyl, trifluoromethyl, amide, and urea.

7. The conjugate of any one of claims 3 to 6, wherein (A1) is selected from:

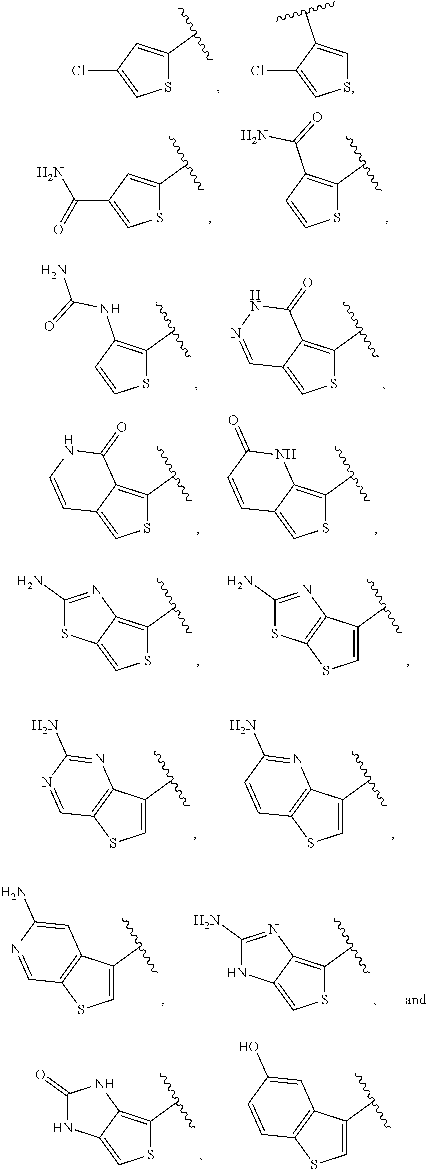

8. The conjugate of any one of claims 3 to 5, wherein (A2) is selected from: 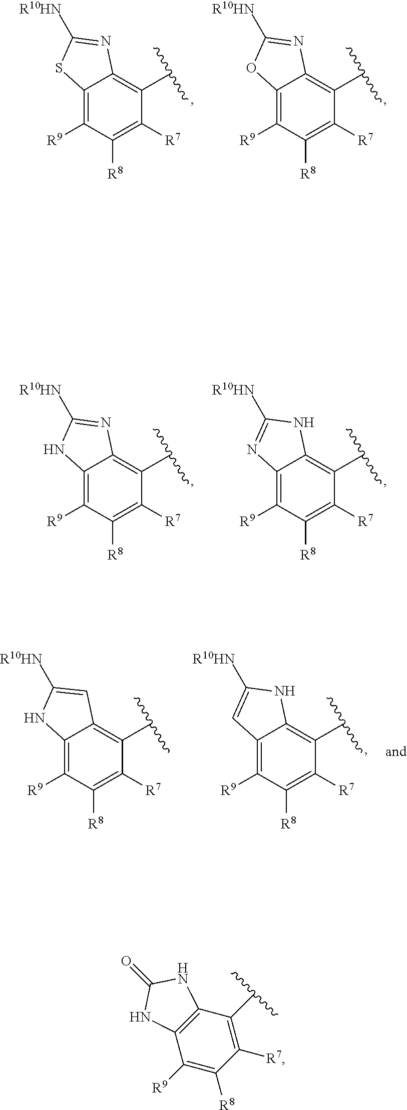
wherein R¹⁴ is CN.

9. The conjugate of claim 8, wherein (A2) is:

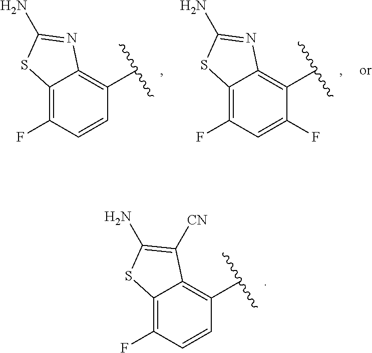 [SMILES: *c1ccc(F)c2sc(N)nc12 ; *c1c(F)cc(F)c2sc(N)nc12 ; *c1ccc(F)c2sc(N)c(C#N)c12]

10. The conjugate of any one of claims 3 to 5, wherein (A3) is selected from:

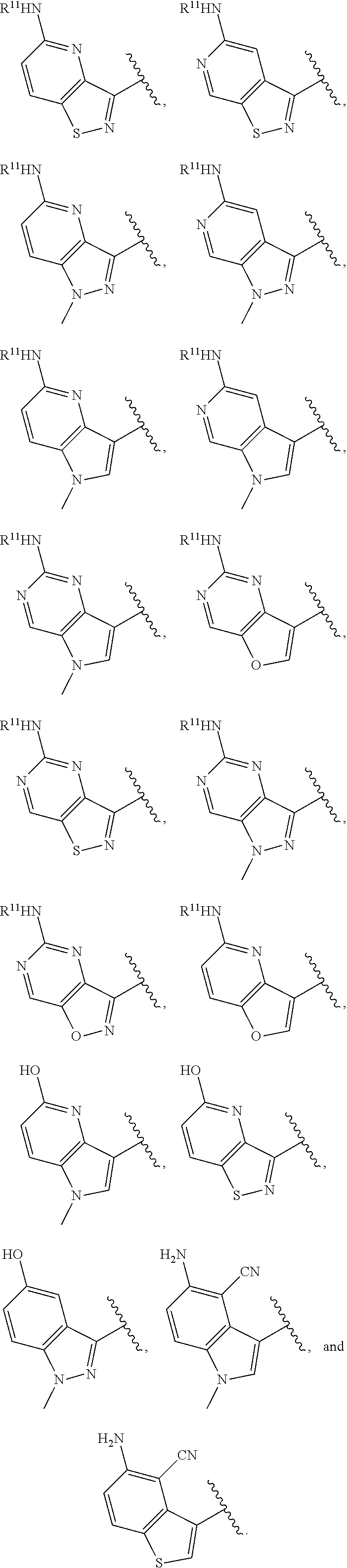

11. The conjugate of an one of claims 3 to 5, wherein (A4) is selected from:

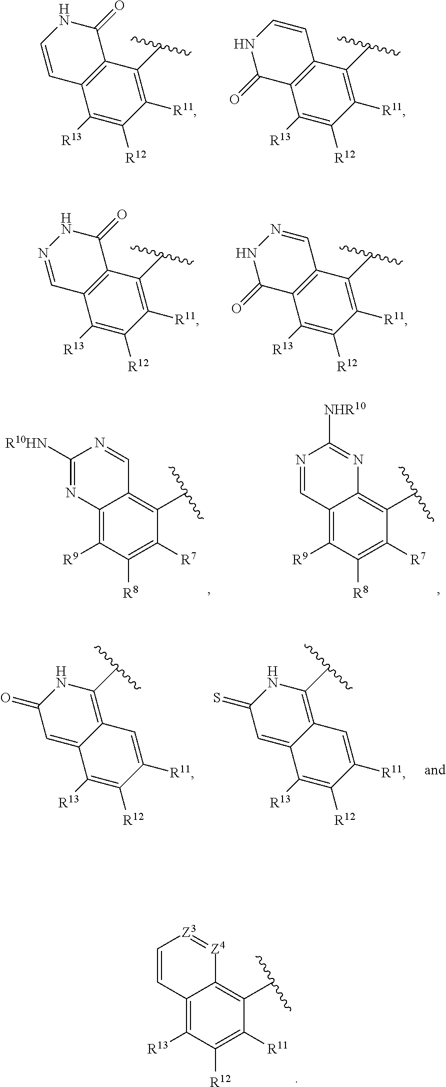

12. The conjugate of claim 11, wherein (A4) is selected from:

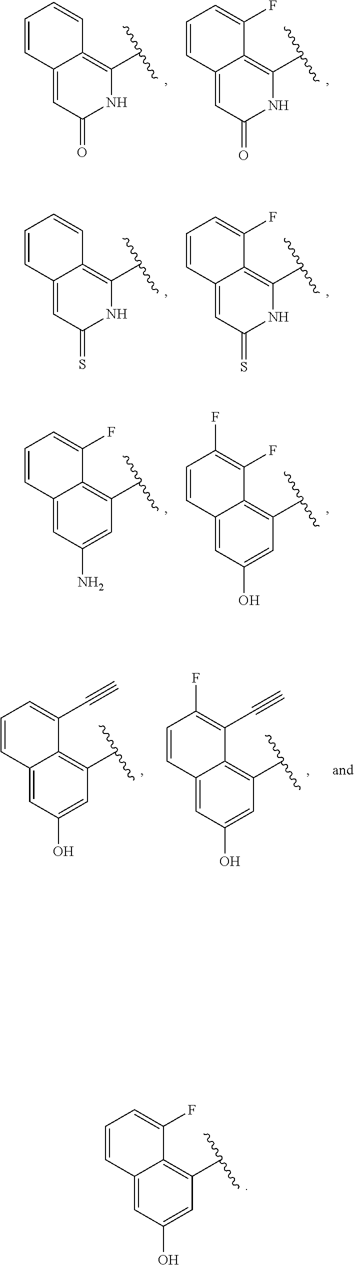

13. The conjugate of any one of claims 3 to 5, wherein Ar is selected from:

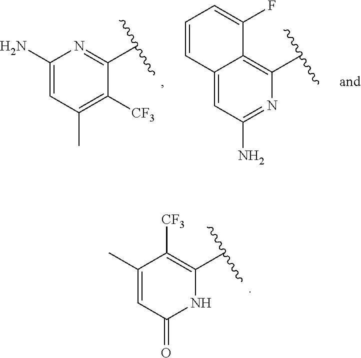 [SMILES: *c1nc(N)cc(C)c1C(F)(F)F ; *c1nc(N)cc2cccc(F)c12 ; *c1[nH]c(=O)cc(C)c1C(F)(F)F]

14. The conjugate of any one of claims 2 to 13, wherein two V form a bridge: —CH₂—CH₂—.

15. The conjugate of any one of claims 2 to 14, wherein G12D- is:

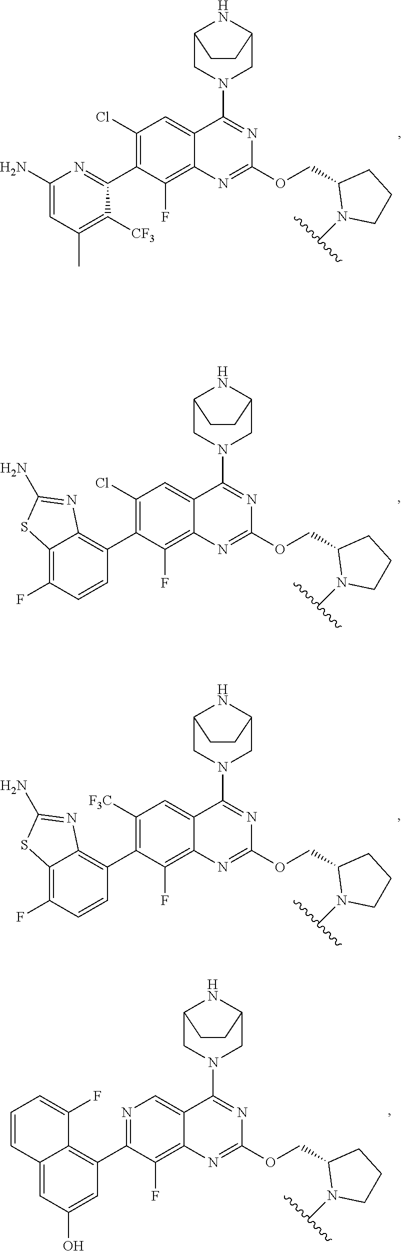

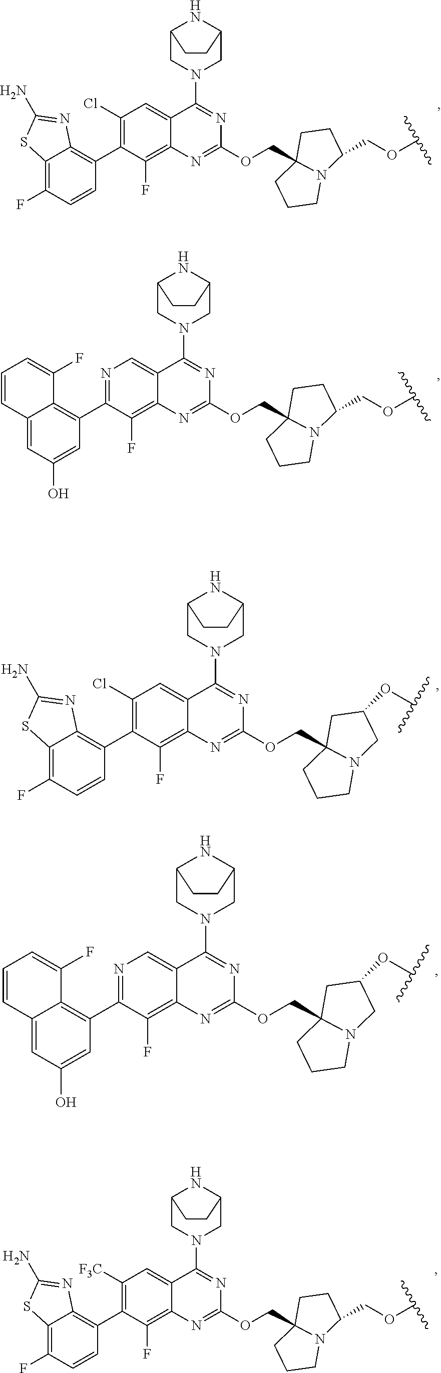 [SMILES: *OC[C@H]1CC[C@@]2(COc3nc(N4CC5CCC(C4)N5)c4cc(Cl)c(-c5ccc(F)c6sc(N)nc56)c(F)c4n3)CCCN12 ; *OC[C@H]1CC[C@@]2(COc3nc(N4CC5CCC(C4)N5)c4cnc(-c5cc(O)cc6cccc(F)c56)c(F)c4n3)CCCN12 ; *O[C@@H]1CN2CCC[C@]2(COc2nc(N3CC4CCC(C3)N4)c3cc(Cl)c(-c4ccc(F)c5sc(N)nc45)c(F)c3n2)C1 ; *O[C@@H]1CN2CCC[C@]2(COc2nc(N3CC4CCC(C3)N4)c3cnc(-c4cc(O)cc5cccc(F)c45)c(F)c3n2)C1 ; *OC[C@H]1CC[C@@]2(COc3nc(N4CC5CCC(C4)N5)c4cc(C(F)(F)F)c(-c5ccc(F)c6sc(N)nc56)c(F)c4n3)CCCN12]

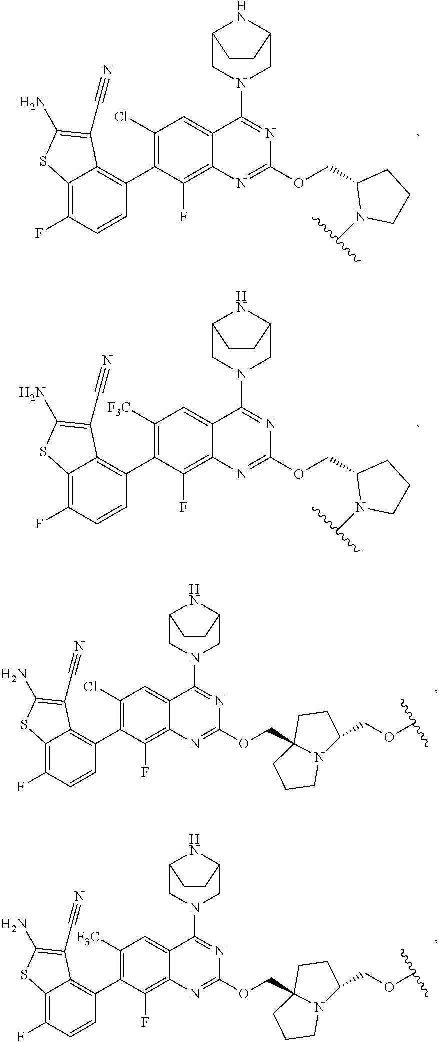 [SMILES: *N1CCC[C@H]1COc1nc(N2CC3CCC(C2)N3)c2cc(Cl)c(-c3ccc(F)c4sc(N)c(C#N)c34)c(F)c2n1 ; *N1CCC[C@H]1COc1nc(N2CC3CCC(C2)N3)c2cc(C(F)(F)F)c(-c3ccc(F)c4sc(N)c(C#N)c34)c(F)c2n1 ; *OC[C@H]1CC[C@@]2(COc3nc(N4CC5CCC(C4)N5)c4cc(Cl)c(-c5ccc(F)c6sc(N)c(C#N)c56)c(F)c4n3)CCCN12 ; *OC[C@H]1CC[C@@]2(COc3nc(N4CC5CCC(C4)N5)c4cc(C(F)(F)F)c(-c5ccc(F)c6sc(N)c(C#N)c56)c(F)c4n3)CCCN12]

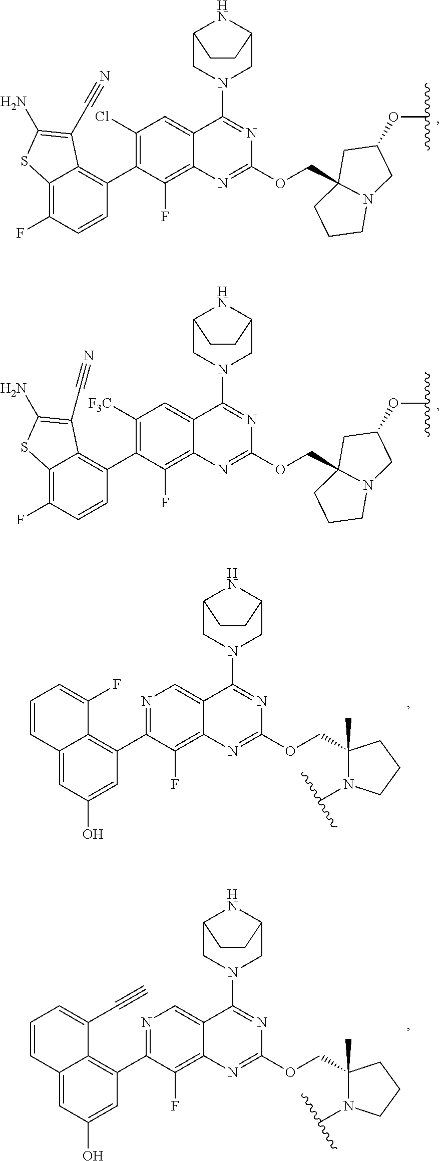 [SMILES: *O[C@@H]1CN2CCC[C@]2(COc2nc(N3CC4CCC(C3)N4)c3cc(Cl)c(-c4ccc(F)c5sc(N)c(C#N)c45)c(F)c3n2)C1 ; *O[C@@H]1CN2CCC[C@]2(COc2nc(N3CC4CCC(C3)N4)c3cc(C(F)(F)F)c(-c4ccc(F)c5sc(N)c(C#N)c45)c(F)c3n2)C1 ; *N1CCC[C@@]1(C)COc1nc(N2CC3CCC(C2)N3)c2cnc(-c3cc(O)cc4cccc(F)c34)c(F)c2n1 ; *N1CCC[C@@]1(C)COc1nc(N2CC3CCC(C2)N3)c2cnc(-c3cc(O)cc4cccc(C#C)c34)c(F)c2n1]

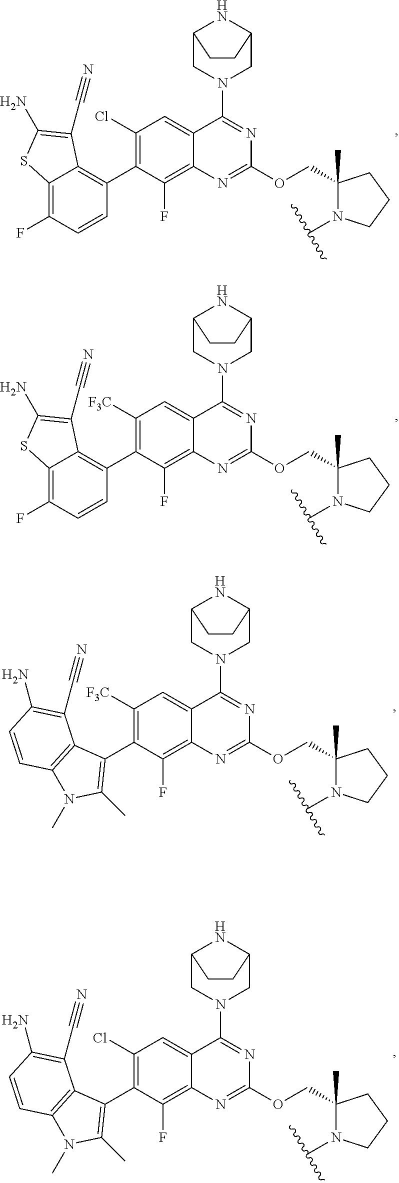 [SMILES: *N1CCC[C@@]1(C)COc1nc(N2CC3CCC(C2)N3)c2cc(Cl)c(-c3ccc(F)c4sc(N)c(C#N)c34)c(F)c2n1 ; *N1CCC[C@@]1(C)COc1nc(N2CC3CCC(C2)N3)c2cc(C(F)(F)F)c(-c3ccc(F)c4sc(N)c(C#N)c34)c(F)c2n1 ; *N1CCC[C@@]1(C)COc1nc(N2CC3CCC(C2)N3)c2cc(C(F)(F)F)c(-c3c(C)n(C)c4ccc(N)c(C#N)c34)c(F)c2n1 ; *N1CCC[C@@]1(C)COc1nc(N2CC3CCC(C2)N3)c2cc(Cl)c(-c3c(C)n(C)c4ccc(N)c(C#N)c34)c(F)c2n1]

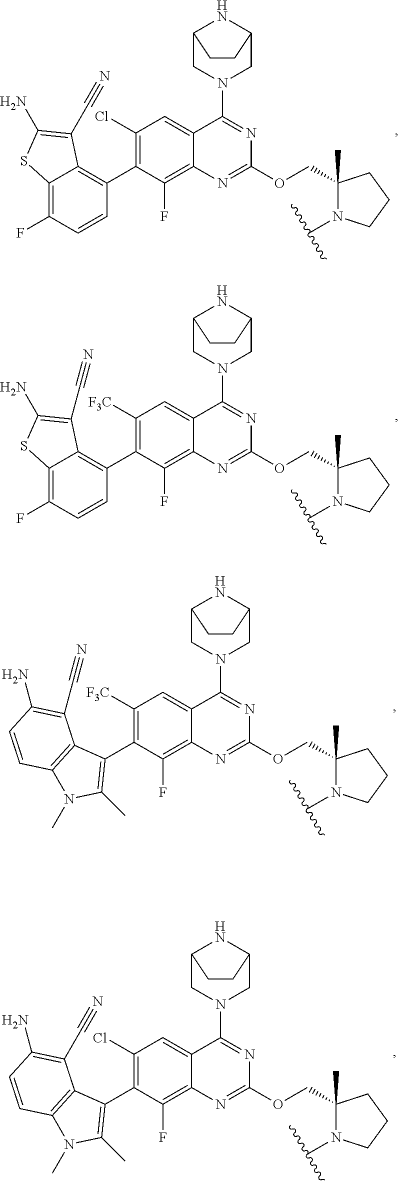 [SMILES: *N1CCC[C@@]1(C)COc1nc(N2CC3CCC(C2)N3)c2cc(Cl)c(-c3ccc(F)c4sc(N)c(C#N)c34)c(F)c2n1 ; *N1CCC[C@@]1(C)COc1nc(N2CC3CCC(C2)N3)c2cc(C(F)(F)F)c(-c3ccc(F)c4sc(N)c(C#N)c34)c(F)c2n1 ; *N1CCC[C@@]1(C)COc1nc(N2CC3CCC(C2)N3)c2cc(C(F)(F)F)c(-c3c(C)n(C)c4ccc(N)c(C#N)c34)c(F)c2n1 ; *N1CCC[C@@]1(C)COc1nc(N2CC3CCC(C2)N3)c2cc(Cl)c(-c3c(C)n(C)c4ccc(N)c(C#N)c34)c(F)c2n1]

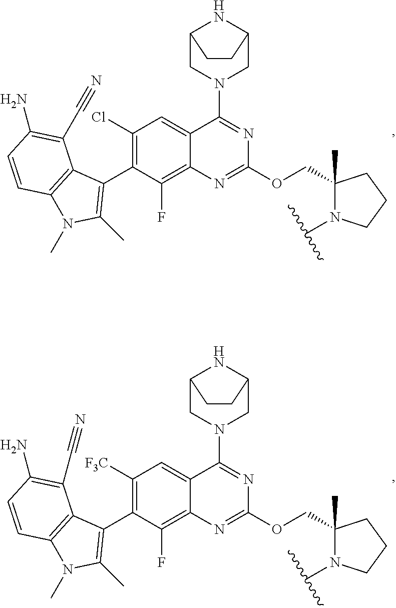 [SMILES: *N1CCC[C@@]1(C)COc1nc(N2CC3CCC(C2)N3)c2cc(Cl)c(-c3c(C)n(C)c4ccc(N)c(C#N)c34)c(F)c2n1 ; *N1CCC[C@@]1(C)COc1nc(N2CC3CCC(C2)N3)c2cc(C(F)(F)F)c(-c3c(C)n(C)c4ccc(N)c(C#N)c34)c(F)c2n1]

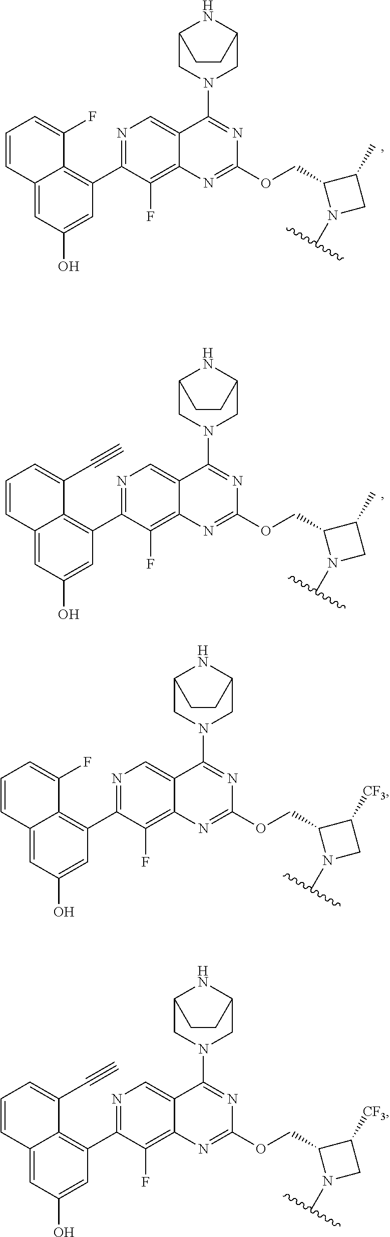 [SMILES: *N1C[C@@H](C)[C@H]1COc1nc(N2CC3CCC(C2)N3)c2cnc(-c3cc(O)cc4cccc(F)c34)c(F)c2n1 ; *N1C[C@@H](C)[C@H]1COc1nc(N2CC3CCC(C2)N3)c2cnc(-c3cc(O)cc4cccc(C#C)c34)c(F)c2n1 ; *N1C[C@@H](C(F)(F)F)[C@H]1COc1nc(N2CC3CCC(C2)N3)c2cnc(-c3cc(O)cc4cccc(F)c34)c(F)c2n1 ; *N1C[C@@H](C(F)(F)F)[C@H]1COc1nc(N2CC3CCC(C2)N3)c2cnc(-c3cc(O)cc4cccc(C#C)c34)c(F)c2n1]

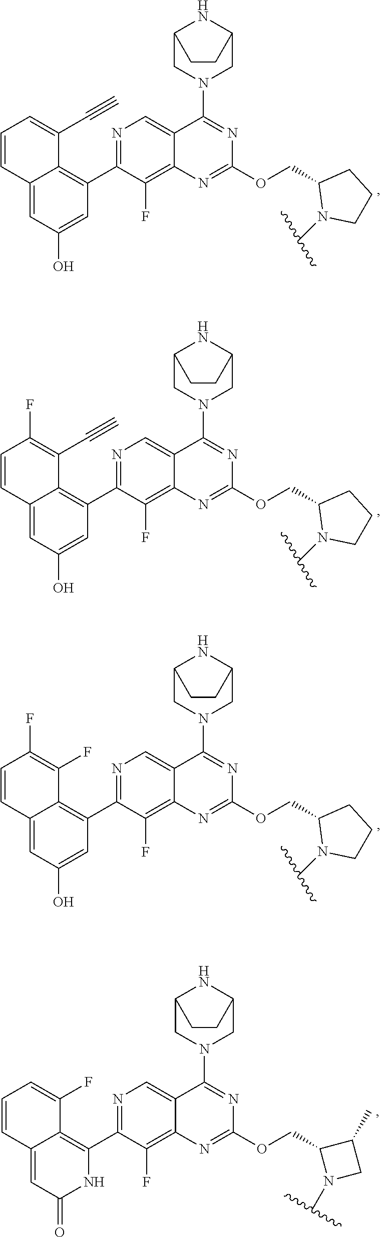 [SMILES: *N1CCC[C@H]1COc1nc(N2CC3CCC(C2)N3)c2cnc(-c3cc(O)cc4cccc(C#C)c34)c(F)c2n1 ; *N1CCC[C@H]1COc1nc(N2CC3CCC(C2)N3)c2cnc(-c3cc(O)cc4ccc(F)c(C#C)c34)c(F)c2n1 ; *N1CCC[C@H]1COc1nc(N2CC3CCC(C2)N3)c2cnc(-c3cc(O)cc4ccc(F)c(F)c34)c(F)c2n1 ; *N1C[C@@H](C)[C@H]1COc1nc(N2CC3CCC(C2)N3)c2cnc(-c3[nH]c(=O)cc4cccc(F)c34)c(F)c2n1]

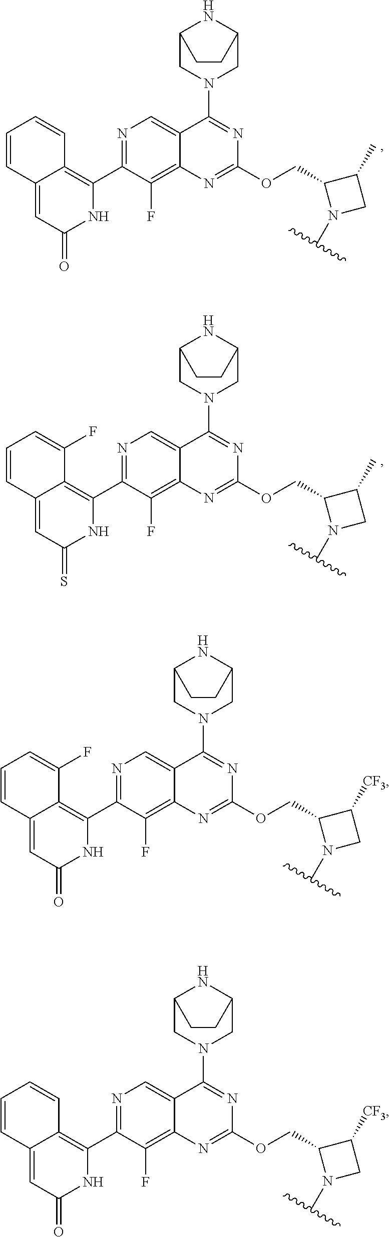 [SMILES: *N1C[C@@H](C)[C@H]1COc1nc(N2CC3CCC(C2)N3)c2cnc(-c3[nH]c(=O)cc4ccccc34)c(F)c2n1 ; *N1C[C@@H](C)[C@H]1COc1nc(N2CC3CCC(C2)N3)c2cnc(-c3[nH]c(=S)cc4cccc(F)c34)c(F)c2n1 ; *N1C[C@@H](C(F)(F)F)[C@H]1COc1nc(N2CC3CCC(C2)N3)c2cnc(-c3[nH]c(=O)cc4cccc(F)c34)c(F)c2n1 ; *N1C[C@@H](C(F)(F)F)[C@H]1COc1nc(N2CC3CCC(C2)N3)c2cnc(-c3[nH]c(=O)cc4ccccc34)c(F)c2n1]

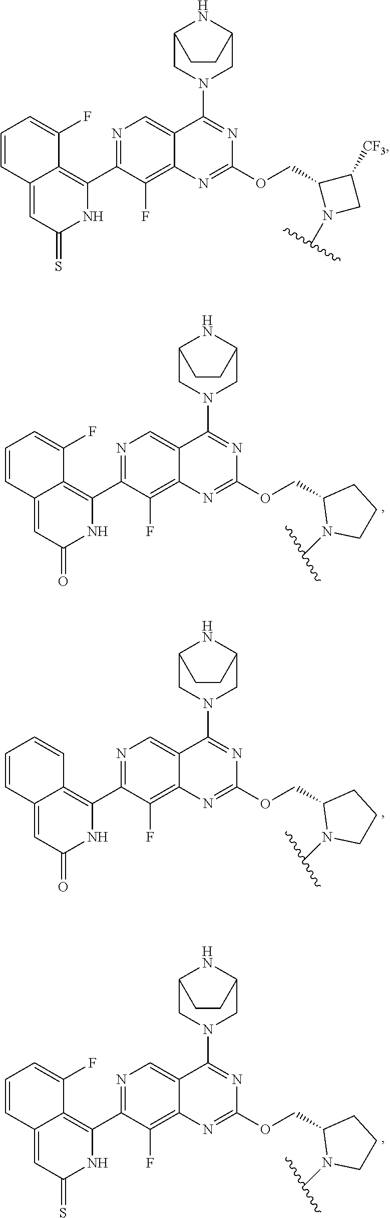 [SMILES: *N1C[C@@H](C(F)(F)F)[C@H]1COc1nc(N2CC3CCC(C2)N3)c2cnc(-c3[nH]c(=S)cc4cccc(F)c34)c(F)c2n1 ; *N1CCC[C@H]1COc1nc(N2CC3CCC(C2)N3)c2cnc(-c3[nH]c(=O)cc4cccc(F)c34)c(F)c2n1 ; *N1CCC[C@H]1COc1nc(N2CC3CCC(C2)N3)c2cnc(-c3[nH]c(=O)cc4ccccc34)c(F)c2n1 ; *N1CCC[C@H]1COc1nc(N2CC3CCC(C2)N3)c2cnc(-c3[nH]c(=S)cc4cccc(F)c34)c(F)c2n1]

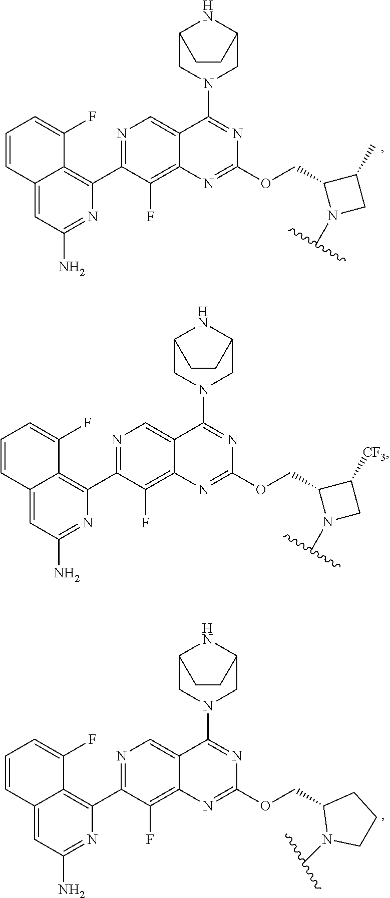 [SMILES: *N1C[C@@H](C)[C@H]1COc1nc(N2CC3CCC(C2)N3)c2cnc(-c3nc(N)cc4cccc(F)c34)c(F)c2n1 ; *N1C[C@@H](C(F)(F)F)[C@H]1COc1nc(N2CC3CCC(C2)N3)c2cnc(-c3nc(N)cc4cccc(F)c34)c(F)c2n1 ; *N1CCC[C@H]1COc1nc(N2CC3CCC(C2)N3)c2cnc(-c3nc(N)cc4cccc(F)c34)c(F)c2n1]

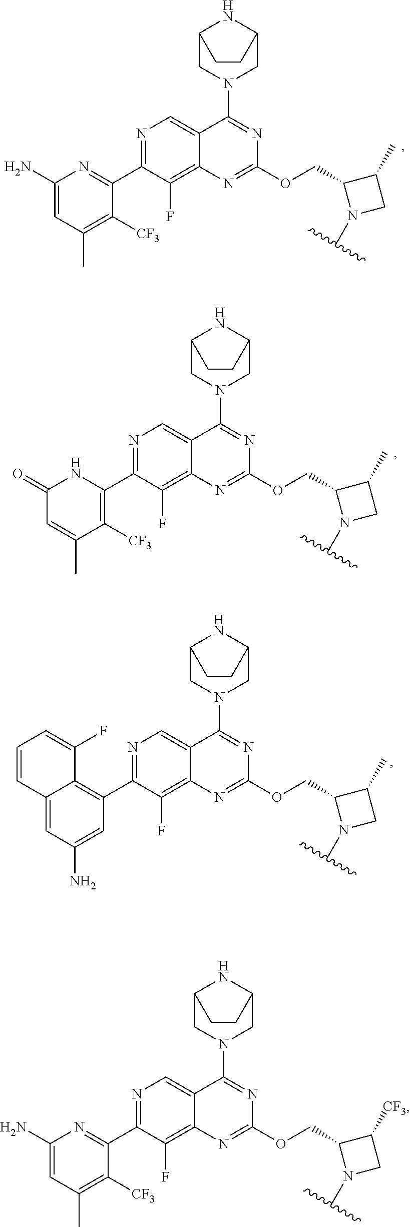 [SMILES: *N1C[C@@H](C)[C@H]1COc1nc(N2CC3CCC(C2)N3)c2cnc(-c3nc(N)cc(C)c3C(F)(F)F)c(F)c2n1 ; *N1C[C@@H](C)[C@H]1COc1nc(N2CC3CCC(C2)N3)c2cnc(-c3[nH]c(=O)cc(C)c3C(F)(F)F)c(F)c2n1 ; *N1C[C@@H](C)[C@H]1COc1nc(N2CC3CCC(C2)N3)c2cnc(-c3cc(N)cc4cccc(F)c34)c(F)c2n1 ; *N1C[C@@H](C(F)(F)F)[C@H]1COc1nc(N2CC3CCC(C2)N3)c2cnc(-c3nc(N)cc(C)c3C(F)(F)F)c(F)c2n1]

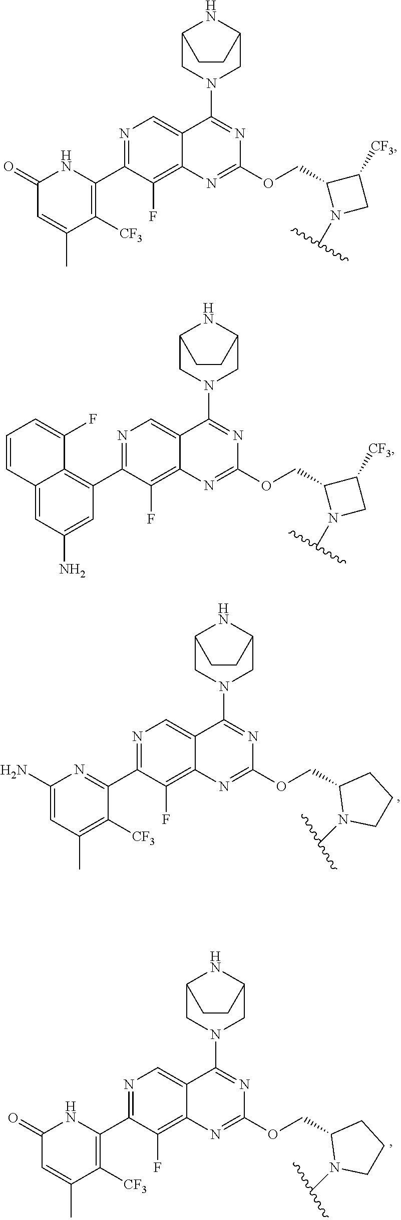 [SMILES: *N1C[C@@H](C(F)(F)F)[C@H]1COc1nc(N2CC3CCC(C2)N3)c2cnc(-c3[nH]c(=O)cc(C)c3C(F)(F)F)c(F)c2n1 ; *N1C[C@@H](C(F)(F)F)[C@H]1COc1nc(N2CC3CCC(C2)N3)c2cnc(-c3cc(N)cc4cccc(F)c34)c(F)c2n1 ; *N1CCC[C@H]1COc1nc(N2CC3CCC(C2)N3)c2cnc(-c3nc(N)cc(C)c3C(F)(F)F)c(F)c2n1 ; *N1CCC[C@H]1COc1nc(N2CC3CCC(C2)N3)c2cnc(-c3[nH]c(=O)cc(C)c3C(F)(F)F)c(F)c2n1]

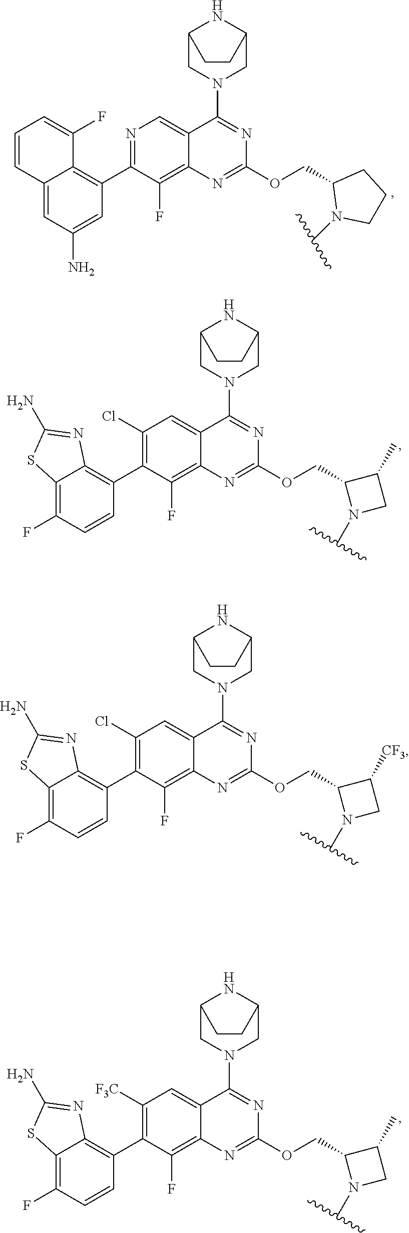 [SMILES: *N1CCC[C@H]1COc1nc(N2CC3CCC(C2)N3)c2cnc(-c3cc(N)cc4cccc(F)c34)c(F)c2n1 ; *N1C[C@@H](C)[C@H]1COc1nc(N2CC3CCC(C2)N3)c2cc(Cl)c(-c3ccc(F)c4sc(N)nc34)c(F)c2n1 ; *N1C[C@@H](C(F)(F)F)[C@H]1COc1nc(N2CC3CCC(C2)N3)c2cc(Cl)c(-c3ccc(F)c4sc(N)nc34)c(F)c2n1 ; *N1C[C@@H](C)[C@H]1COc1nc(N2CC3CCC(C2)N3)c2cc(C(F)(F)F)c(-c3ccc(F)c4sc(N)nc34)c(F)c2n1]

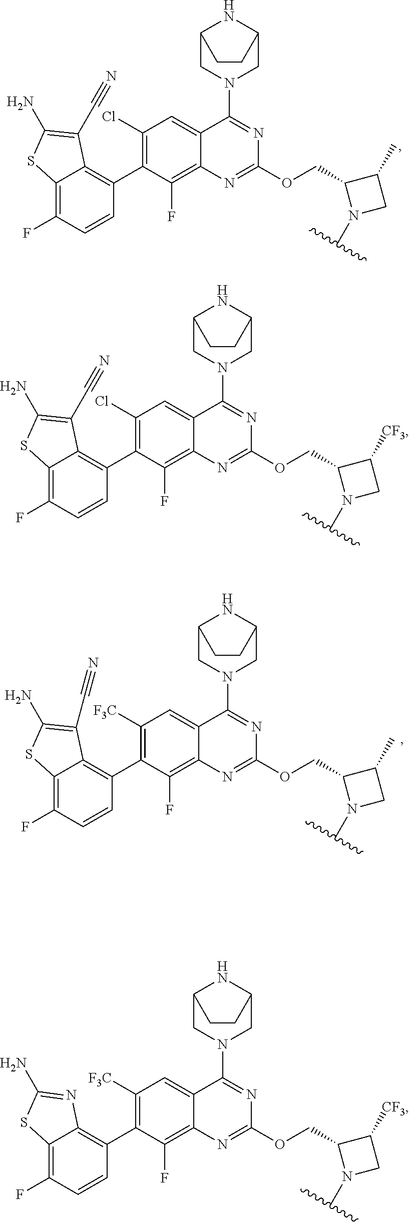 [SMILES: *N1C[C@@H](C)[C@H]1COc1nc(N2CC3CCC(C2)N3)c2cc(Cl)c(-c3ccc(F)c4sc(N)c(C#N)c34)c(F)c2n1 ; *N1C[C@@H](C(F)(F)F)[C@H]1COc1nc(N2CC3CCC(C2)N3)c2cc(Cl)c(-c3ccc(F)c4sc(N)c(C#N)c34)c(F)c2n1 ; *N1C[C@@H](C)[C@H]1COc1nc(N2CC3CCC(C2)N3)c2cc(C(F)(F)F)c(-c3ccc(F)c4sc(N)c(C#N)c34)c(F)c2n1 ; *N1C[C@@H](C(F)(F)F)[C@H]1COc1nc(N2CC3CCC(C2)N3)c2cc(C(F)(F)F)c(-c3ccc(F)c4sc(N)nc34)c(F)c2n1]

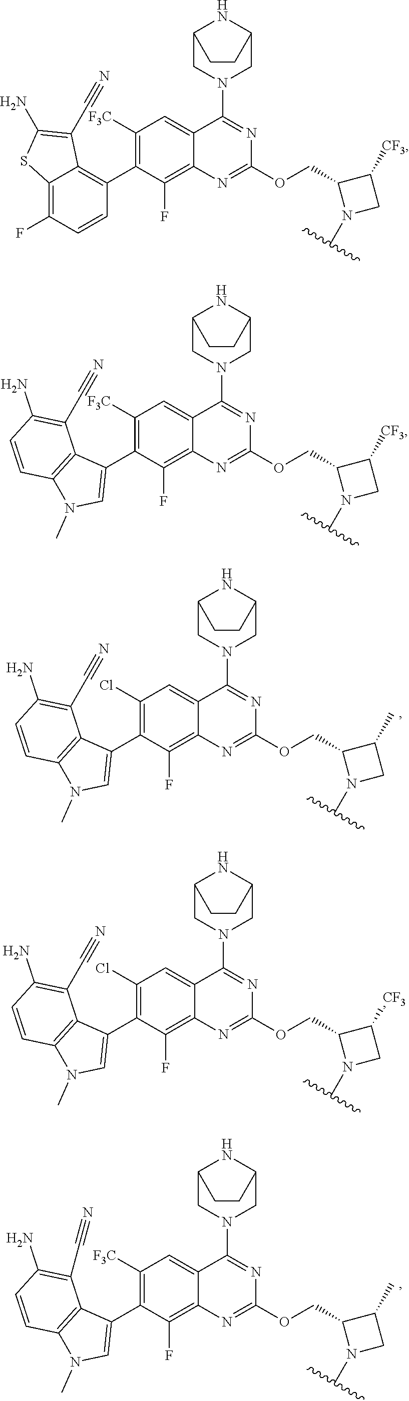 [SMILES: *N1C[C@@H](C(F)(F)F)[C@H]1COc1nc(N2CC3CCC(C2)N3)c2cc(C(F)(F)F)c(-c3ccc(F)c4sc(N)c(C#N)c34)c(F)c2n1 ; *N1C[C@@H](C(F)(F)F)[C@H]1COc1nc(N2CC3CCC(C2)N3)c2cc(C(F)(F)F)c(-c3cn(C)c4ccc(N)c(C#N)c34)c(F)c2n1 ; *N1C[C@@H](C)[C@H]1COc1nc(N2CC3CCC(C2)N3)c2cc(Cl)c(-c3cn(C)c4ccc(N)c(C#N)c34)c(F)c2n1 ; *N1C[C@@H](C(F)(F)F)[C@H]1COc1nc(N2CC3CCC(C2)N3)c2cc(Cl)c(-c3cn(C)c4ccc(N)c(C#N)c34)c(F)c2n1 ; *N1C[C@@H](C)[C@H]1COc1nc(N2CC3CCC(C2)N3)c2cc(C(F)(F)F)c(-c3cn(C)c4ccc(N)c(C#N)c34)c(F)c2n1]

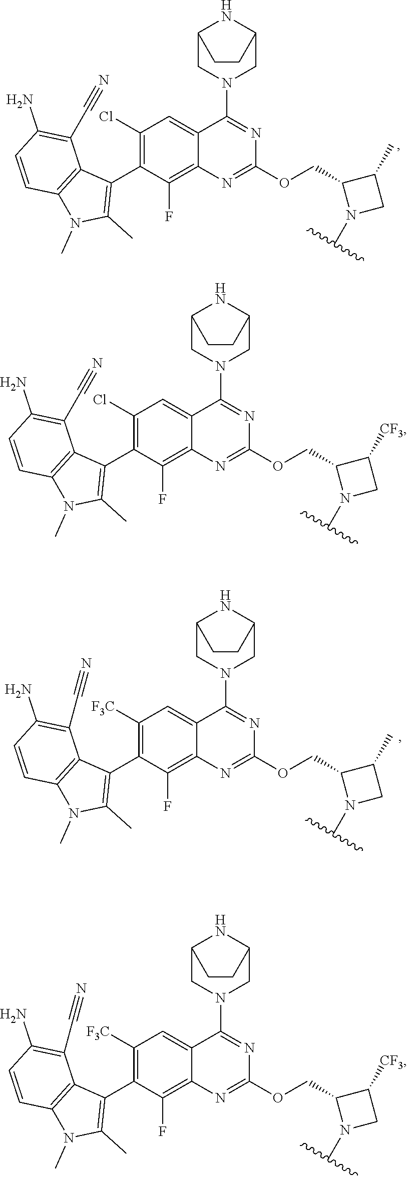 [SMILES: *N1C[C@@H](C)[C@H]1COc1nc(N2CC3CCC(C2)N3)c2cc(Cl)c(-c3c(C)n(C)c4ccc(N)c(C#N)c34)c(F)c2n1 ; *N1C[C@@H](C(F)(F)F)[C@H]1COc1nc(N2CC3CCC(C2)N3)c2cc(Cl)c(-c3c(C)n(C)c4ccc(N)c(C#N)c34)c(F)c2n1 ; *N1C[C@@H](C)[C@H]1COc1nc(N2CC3CCC(C2)N3)c2cc(C(F)(F)F)c(-c3c(C)n(C)c4ccc(N)c(C#N)c34)c(F)c2n1 ; *N1C[C@@H](C(F)(F)F)[C@H]1COc1nc(N2CC3CCC(C2)N3)c2cc(C(F)(F)F)c(-c3c(C)n(C)c4ccc(N)c(C#N)c34)c(F)c2n1]

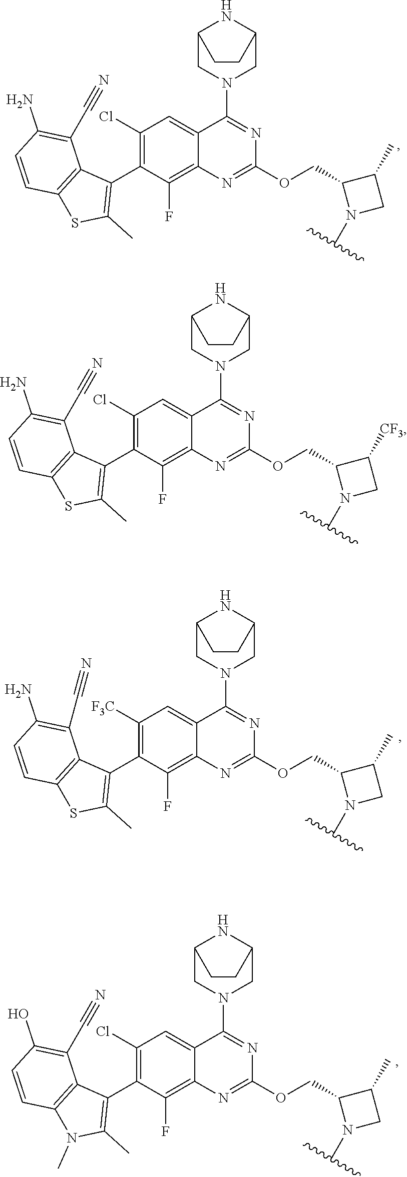 [SMILES: *N1C[C@@H](C)[C@H]1COc1nc(N2CC3CCC(C2)N3)c2cc(Cl)c(-c3c(C)sc4ccc(N)c(C#N)c34)c(F)c2n1 ; *N1C[C@@H](C(F)(F)F)[C@H]1COc1nc(N2CC3CCC(C2)N3)c2cc(Cl)c(-c3c(C)sc4ccc(N)c(C#N)c34)c(F)c2n1 ; *N1C[C@@H](C)[C@H]1COc1nc(N2CC3CCC(C2)N3)c2cc(C(F)(F)F)c(-c3c(C)sc4ccc(N)c(C#N)c34)c(F)c2n1 ; *N1C[C@@H](C)[C@H]1COc1nc(N2CC3CCC(C2)N3)c2cc(Cl)c(-c3c(C)n(C)c4ccc(O)c(C#N)c34)c(F)c2n1]

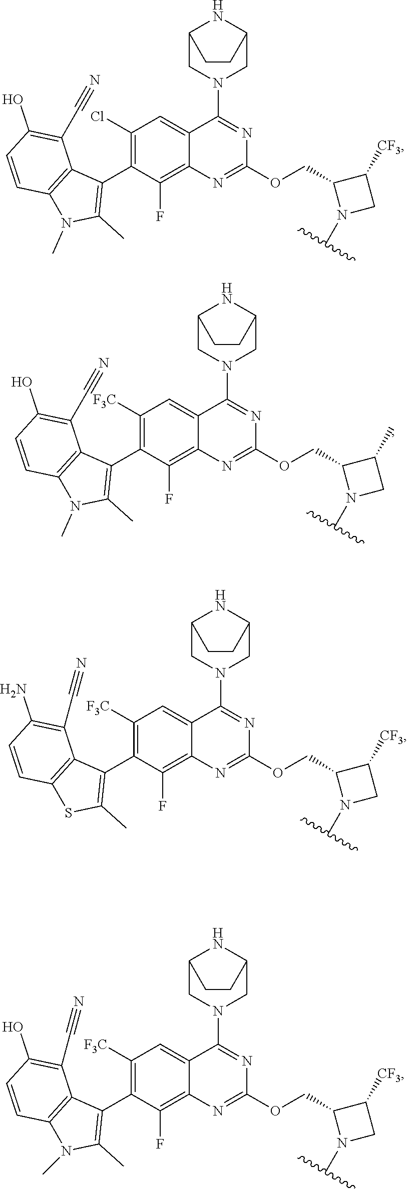 [SMILES: *N1C[C@@H](C(F)(F)F)[C@H]1COc1nc(N2CC3CCC(C2)N3)c2cc(Cl)c(-c3c(C)n(C)c4ccc(O)c(C#N)c34)c(F)c2n1 ; *N1C[C@@H](C)[C@H]1COc1nc(N2CC3CCC(C2)N3)c2cc(C(F)(F)F)c(-c3c(C)n(C)c4ccc(O)c(C#N)c34)c(F)c2n1 ; *N1C[C@@H](C(F)(F)F)[C@H]1COc1nc(N2CC3CCC(C2)N3)c2cc(C(F)(F)F)c(-c3c(C)sc4ccc(N)c(C#N)c34)c(F)c2n1 ; *N1C[C@@H](C(F)(F)F)[C@H]1COc1nc(N2CC3CCC(C2)N3)c2cc(C(F)(F)F)c(-c3c(C)n(C)c4ccc(O)c(C#N)c34)c(F)c2n1]

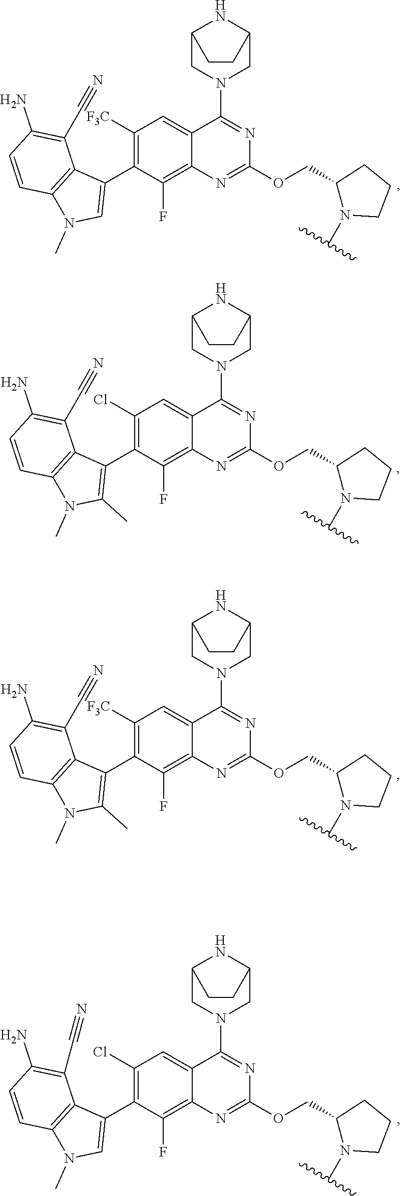 [SMILES: *N1CCC[C@H]1COc1nc(N2CC3CCC(C2)N3)c2cc(C(F)(F)F)c(-c3cn(C)c4ccc(N)c(C#N)c34)c(F)c2n1 ; *N1CCC[C@H]1COc1nc(N2CC3CCC(C2)N3)c2cc(Cl)c(-c3c(C)n(C)c4ccc(N)c(C#N)c34)c(F)c2n1 ; *N1CCC[C@H]1COc1nc(N2CC3CCC(C2)N3)c2cc(C(F)(F)F)c(-c3c(C)n(C)c4ccc(N)c(C#N)c34)c(F)c2n1 ; *N1CCC[C@H]1COc1nc(N2CC3CCC(C2)N3)c2cc(Cl)c(-c3cn(C)c4ccc(N)c(C#N)c34)c(F)c2n1]

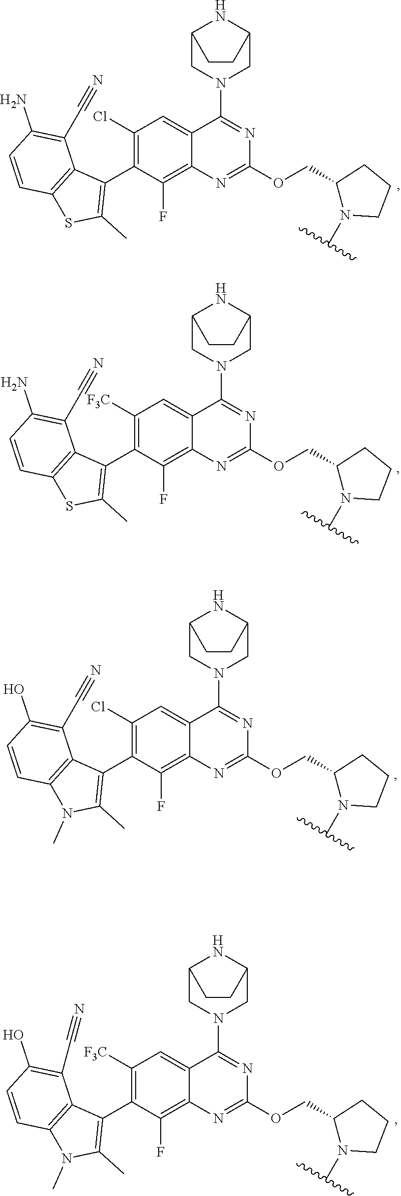 [SMILES: *N1CCC[C@H]1COc1nc(N2CC3CCC(C2)N3)c2cc(Cl)c(-c3c(C)sc4ccc(N)c(C#N)c34)c(F)c2n1 ; *N1CCC[C@H]1COc1nc(N2CC3CCC(C2)N3)c2cc(C(F)(F)F)c(-c3c(C)sc4ccc(N)c(C#N)c34)c(F)c2n1 ; *N1CCC[C@H]1COc1nc(N2CC3CCC(C2)N3)c2cc(Cl)c(-c3c(C)n(C)c4ccc(O)c(C#N)c34)c(F)c2n1 ; *N1CCC[C@H]1COc1nc(N2CC3CCC(C2)N3)c2cc(C(F)(F)F)c(-c3c(C)n(C)c4ccc(O)c(C#N)c34)c(F)c2n1]

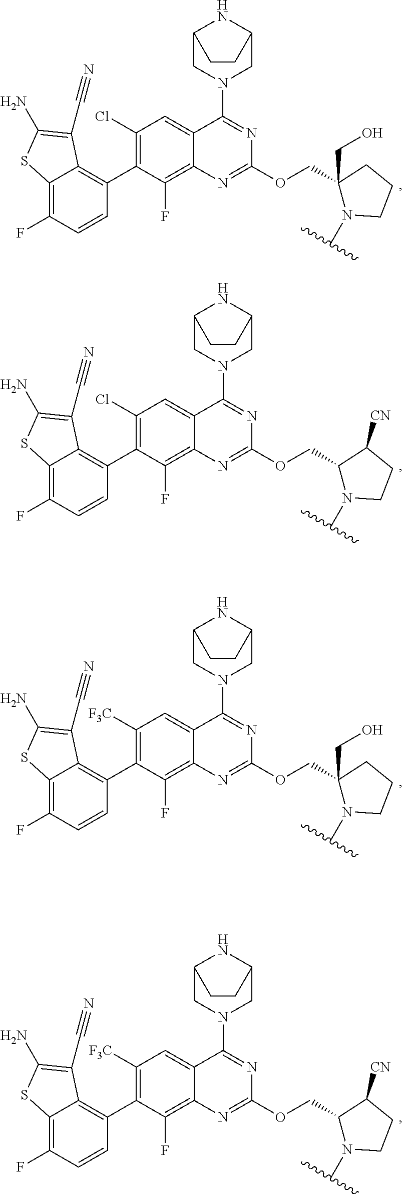 [SMILES: *N1CCC[C@@]1(CO)COc1nc(N2CC3CCC(C2)N3)c2cc(Cl)c(-c3ccc(F)c4sc(N)c(C#N)c34)c(F)c2n1 ; *N1CC[C@H](C#N)[C@H]1COc1nc(N2CC3CCC(C2)N3)c2cc(Cl)c(-c3ccc(F)c4sc(N)c(C#N)c34)c(F)c2n1 ; *N1CCC[C@@]1(CO)COc1nc(N2CC3CCC(C2)N3)c2cc(C(F)(F)F)c(-c3ccc(F)c4sc(N)c(C#N)c34)c(F)c2n1 ; *N1CC[C@H](C#N)[C@H]1COc1nc(N2CC3CCC(C2)N3)c2cc(C(F)(F)F)c(-c3ccc(F)c4sc(N)c(C#N)c34)c(F)c2n1]

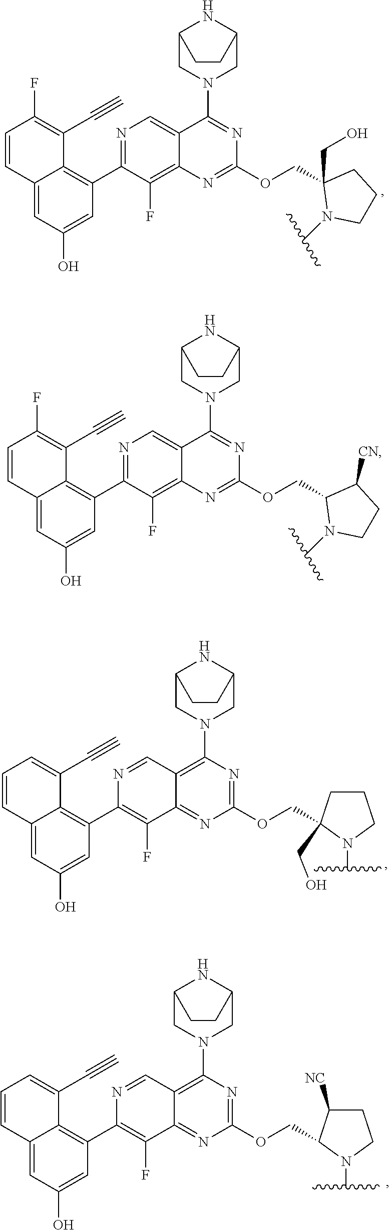 [SMILES: *N1CCC[C@@]1(CO)COc1nc(N2CC3CCC(C2)N3)c2cnc(-c3cc(O)cc4ccc(F)c(C#C)c34)c(F)c2n1 ; *N1CC[C@H](C#N)[C@H]1COc1nc(N2CC3CCC(C2)N3)c2cnc(-c3cc(O)cc4ccc(F)c(C#C)c34)c(F)c2n1 ; *N1CCC[C@@]1(CO)COc1nc(N2CC3CCC(C2)N3)c2cnc(-c3cc(O)cc4cccc(C#C)c34)c(F)c2n1 ; *N1CC[C@H](C#N)[C@H]1COc1nc(N2CC3CCC(C2)N3)c2cnc(-c3cc(O)cc4cccc(C#C)c34)c(F)c2n1]

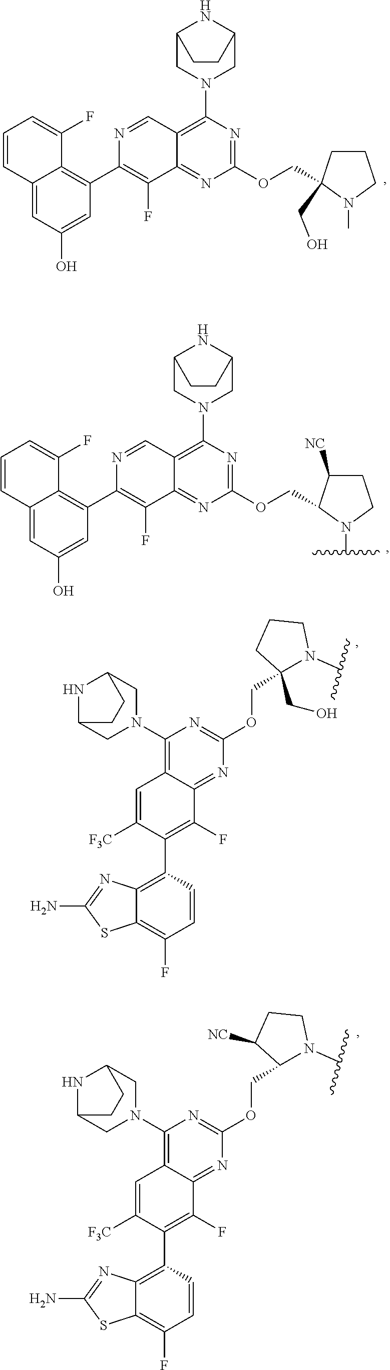

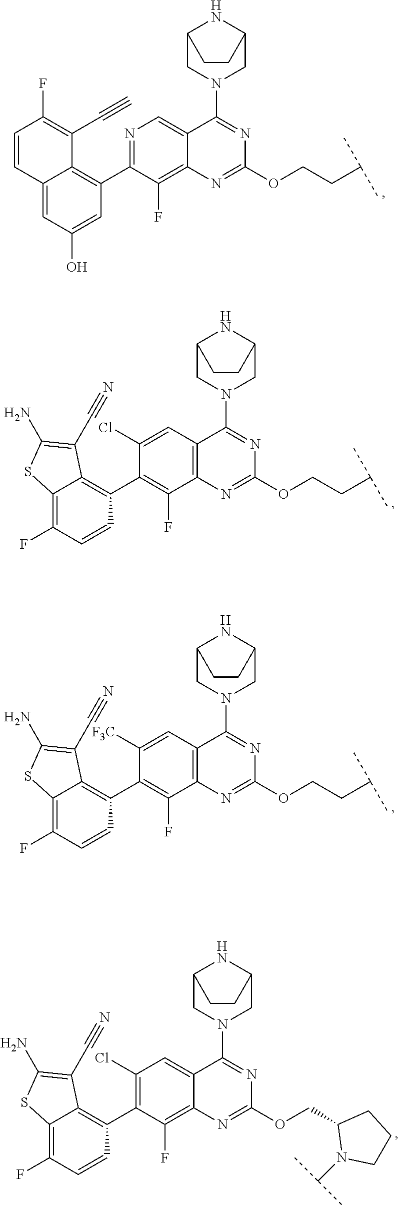

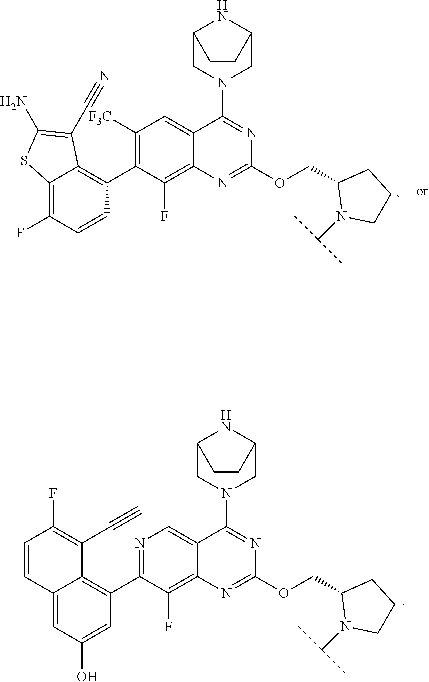

16. The conjugate of any one of claims 2 to 15, wherein -L- is selected from: 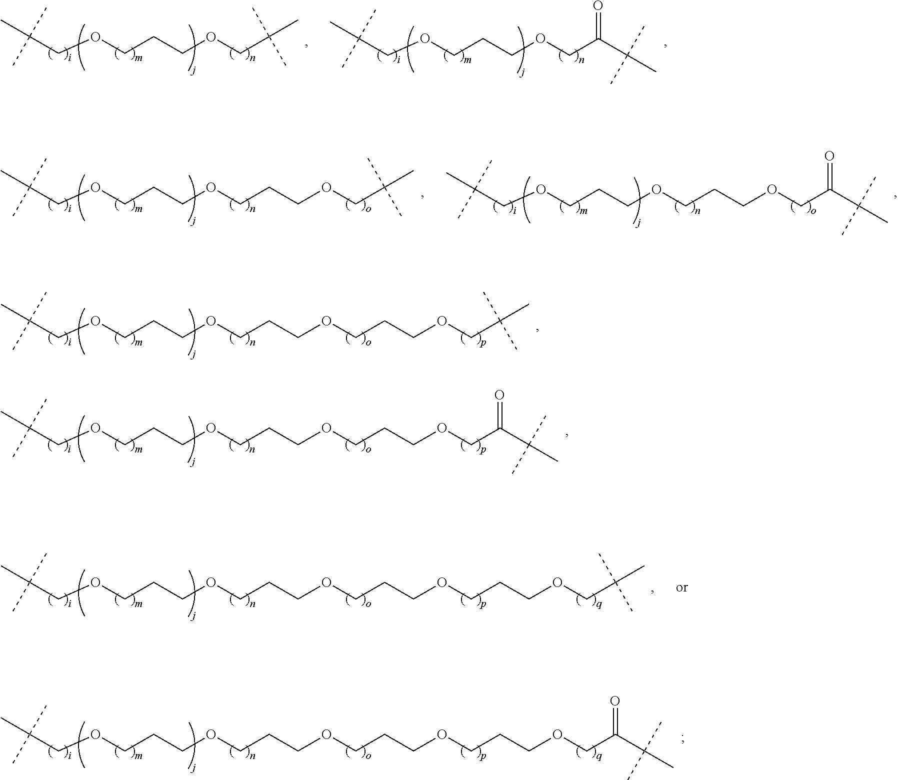
wherein any number of carbon or oxygen atoms of L are optionally exchanged with a heteroatom selected from NR^{L} S, or SO₂ atoms, where R^{L} is hydrogen or methyl; and i, j, m, n, o, p, q are integers independently selected from 0, 1, 2, 3, 4, 5, 6, 7, 8, 9 and 10; and wherein each carbon atom of L is optionally substituted with oxo (═O), OH, halo, NR₃R₄, C₁₋₄ alkyl, C₁₋₄ haloalkyl, C₁₋₄ alkoxy, C₃₋₄ cycloalkyl; wherein each L is attached to Z′ and ubiquitin binding moiety (UBM) in either orientation.

17. The conjugate of any one of claims 2 to 15, wherein -L- is selected from: 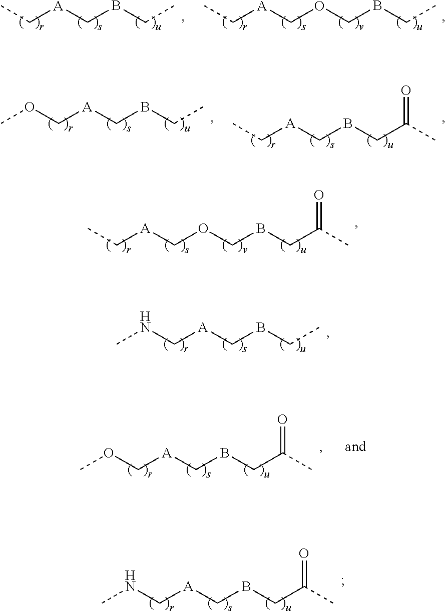
wherein each A and B are independently selected from absent, a 3 to 10 membered cycloalkylene, 4- to 10-membered heterocycloalkylene, 5- to 10-membered heteroarylene and 6- to 10-membered arylene, wherein any number of carbon or oxygen atoms of L are optionally exchanged with a heteroatom selected from NR^{L}, S or SO₂ atoms, where R^{L} is hydrogen or methyl; r, s, u, v are integers independently selected from 0, 1, 2, 3, 4, 5, 6, 7, 8, 9 and 10; wherein each carbon atom of L is optionally substituted with oxo (═O), OH, halo, NR₃R₄, C₁₋₄ alkyl, C₁₋₄ haloalkyl, C₁₋₄ alkoxy, C₃₋₄ cycloalkyl; wherein each L is attached to Z′ and ubiquitin binding moiety (UBM) in either orientation; and
when A and B are a 3- to 10-membered cycloalkyl or 4- to 10-membered heterocycloalkyl, they are incorporated at any two independent ring atoms, or at the same ring atom.

18. The conjugate of any one of claims 2 to 17, wherein L is selected from:

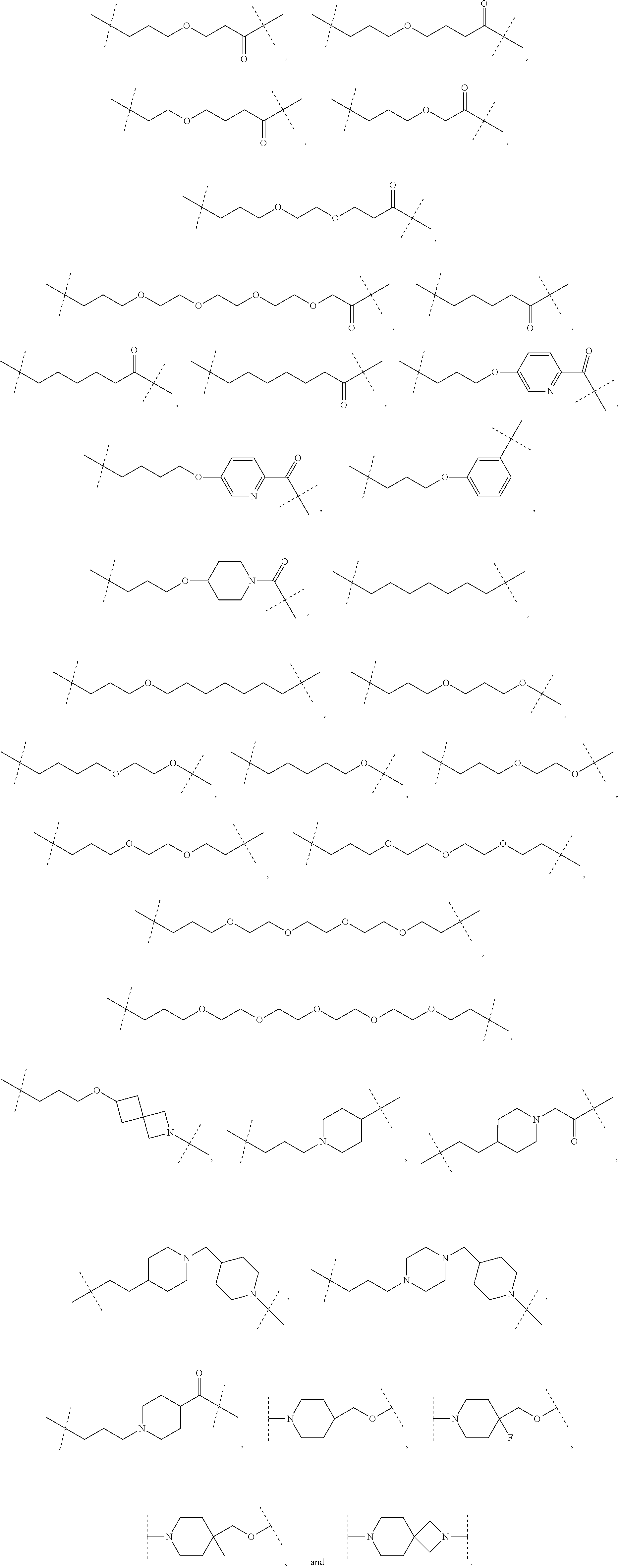

19. The conjugate of any one of claims 2 to 15, wherein L is a structure according to formula (L1) or (L2): 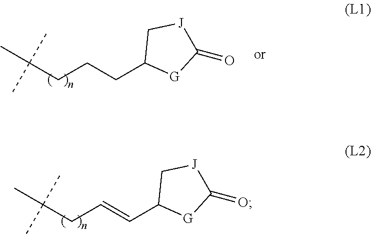
J is null, —CH₂—, O, —OCH₂—, —CH₂O—, —CH₂CH₂—;
G is a nitrogen attached to ubiquitin binding motiety (UBM); and
n is an integer selected from 0, 1, and 2.

20. The conjugate of claim 19, wherein L is selected from:

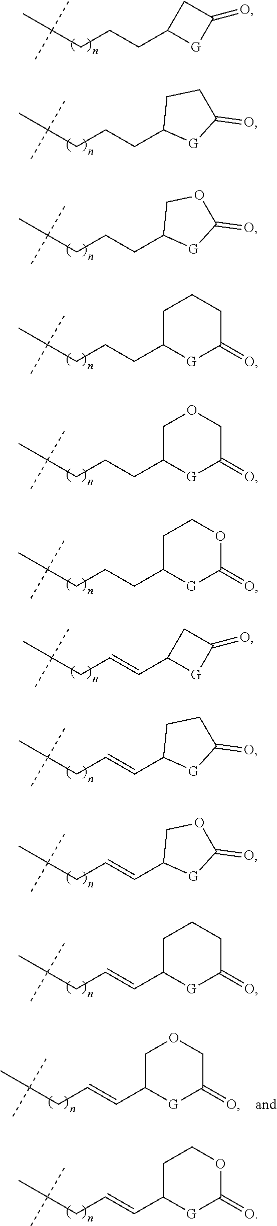

21. The conjugate of claim 19 or 20, wherein n is 1.

22. The conjugate of any one of claims 1 to 21, wherein the UBM is a von Hippel-Lindau (VHL) or cereblon ligand.

23. The conjugate of claim 22, wherein the VHL ligand is selected from:

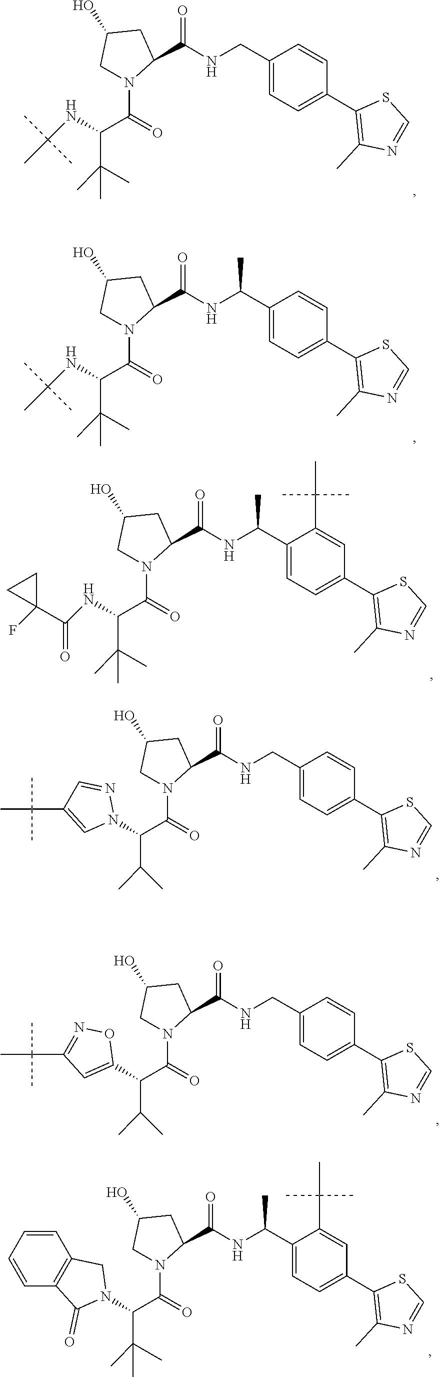

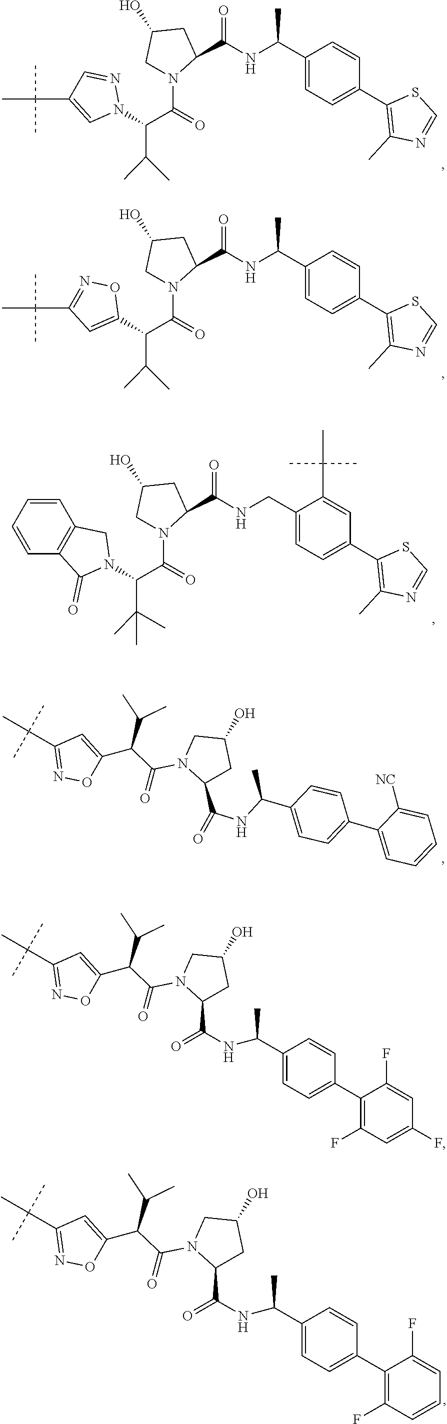

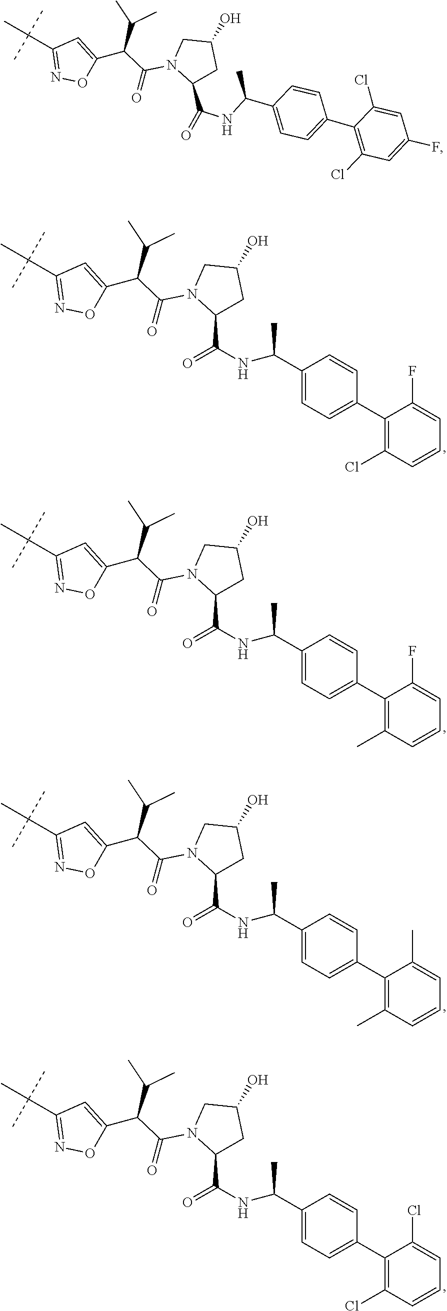

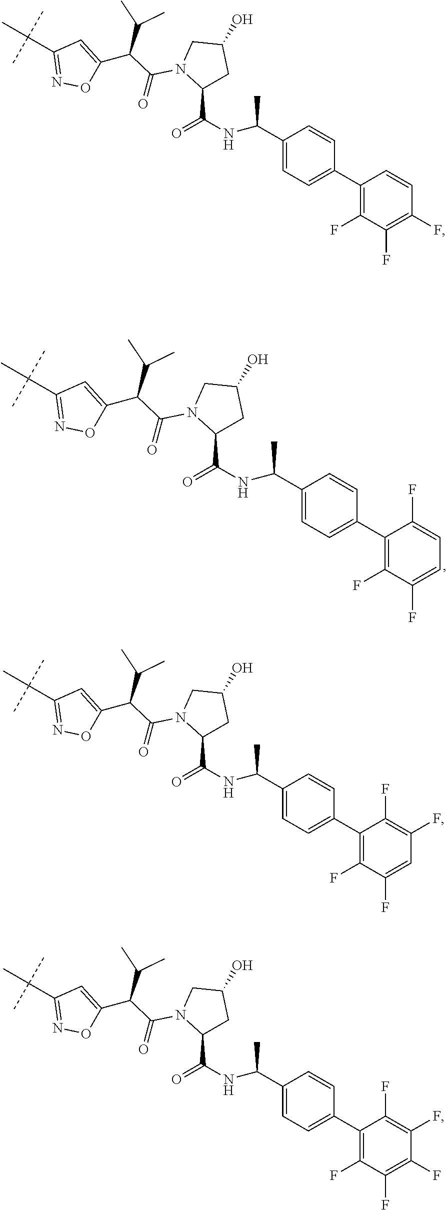

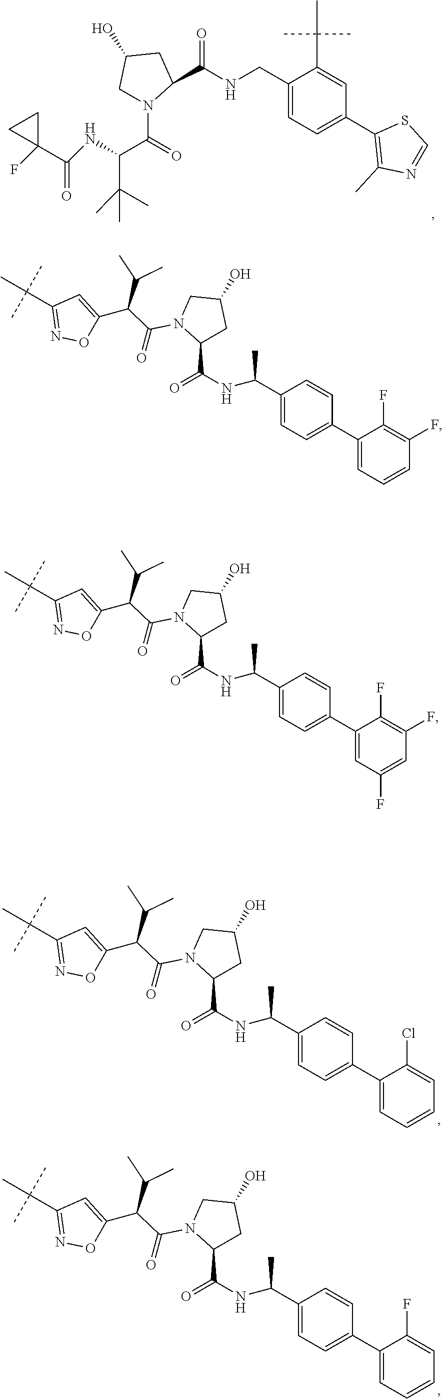

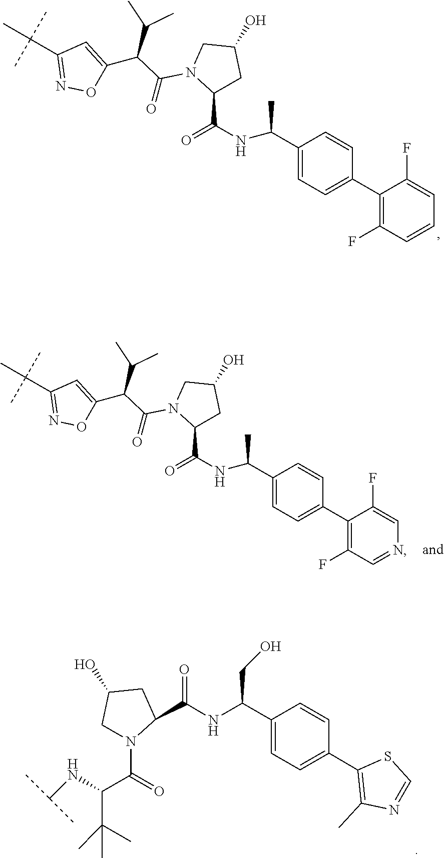

24. The conjugate of claim 22, wherein the VHL ligand is selected from:

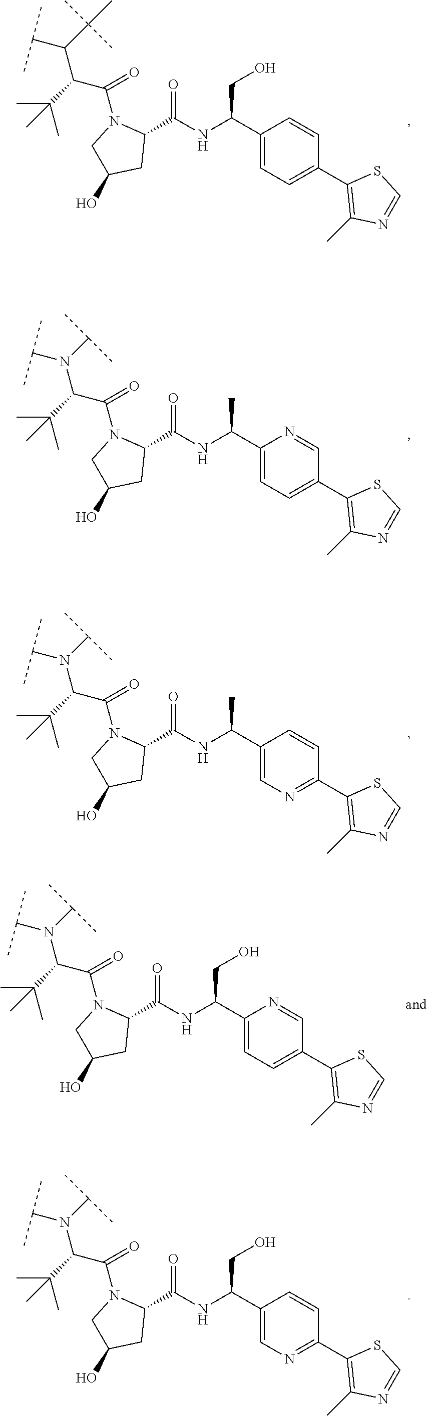

25. The conjugate of claim 22, wherein the cereblon ligand is selected from:

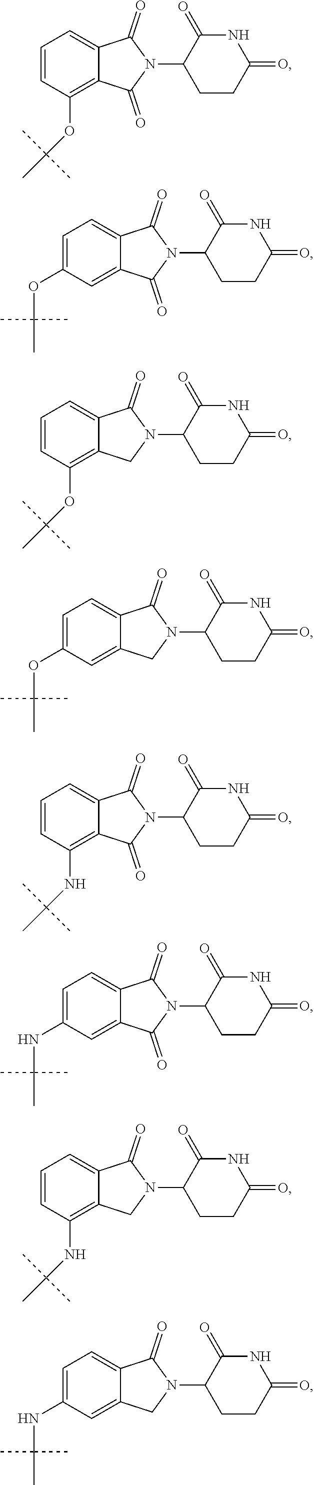

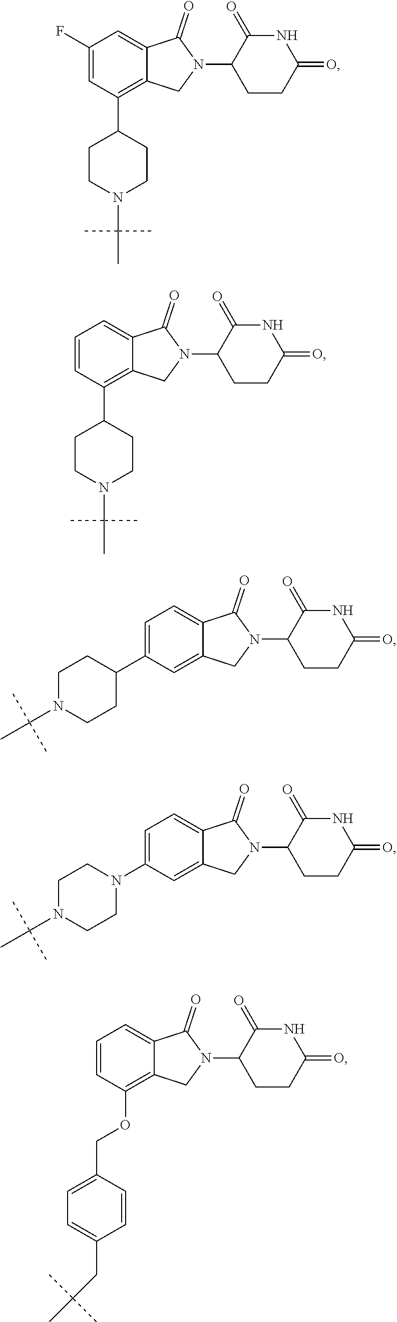

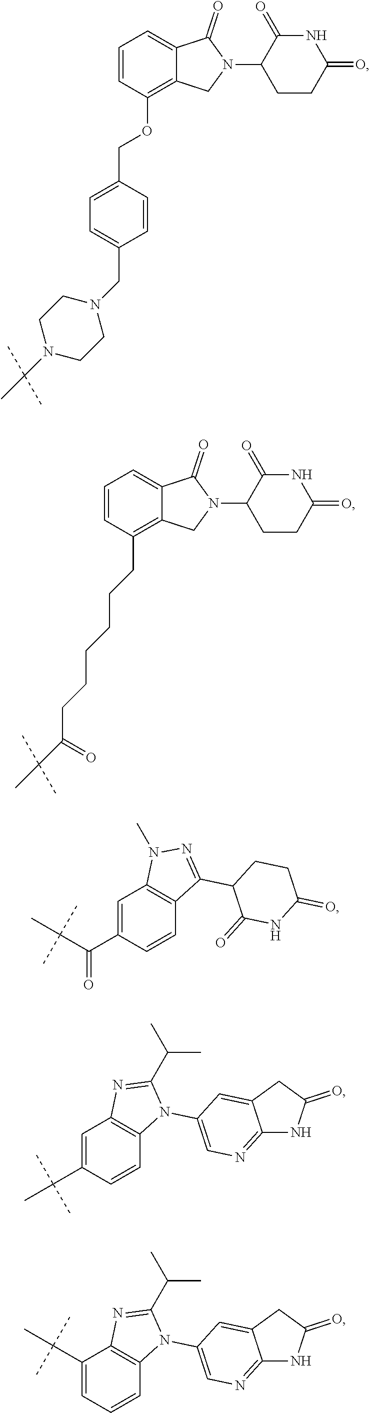

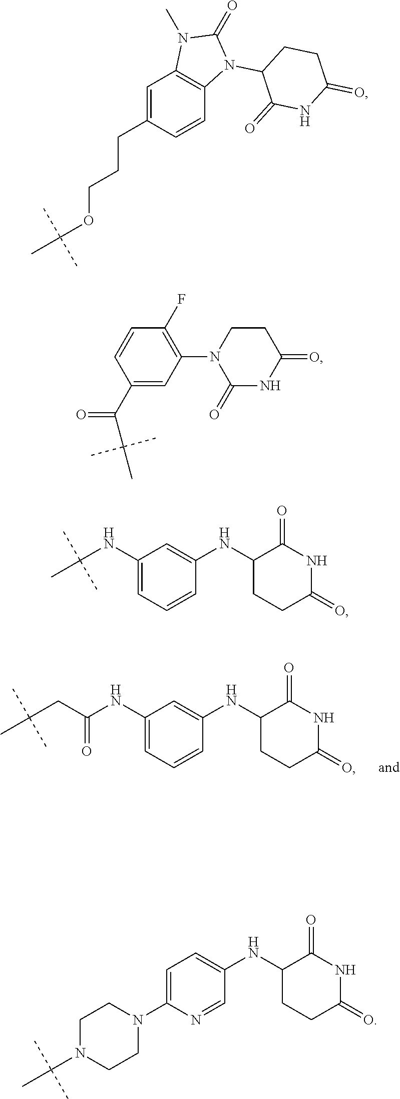

26. The conjugate of any one of claims 1 to 24, wherein the conjugate is selected from:

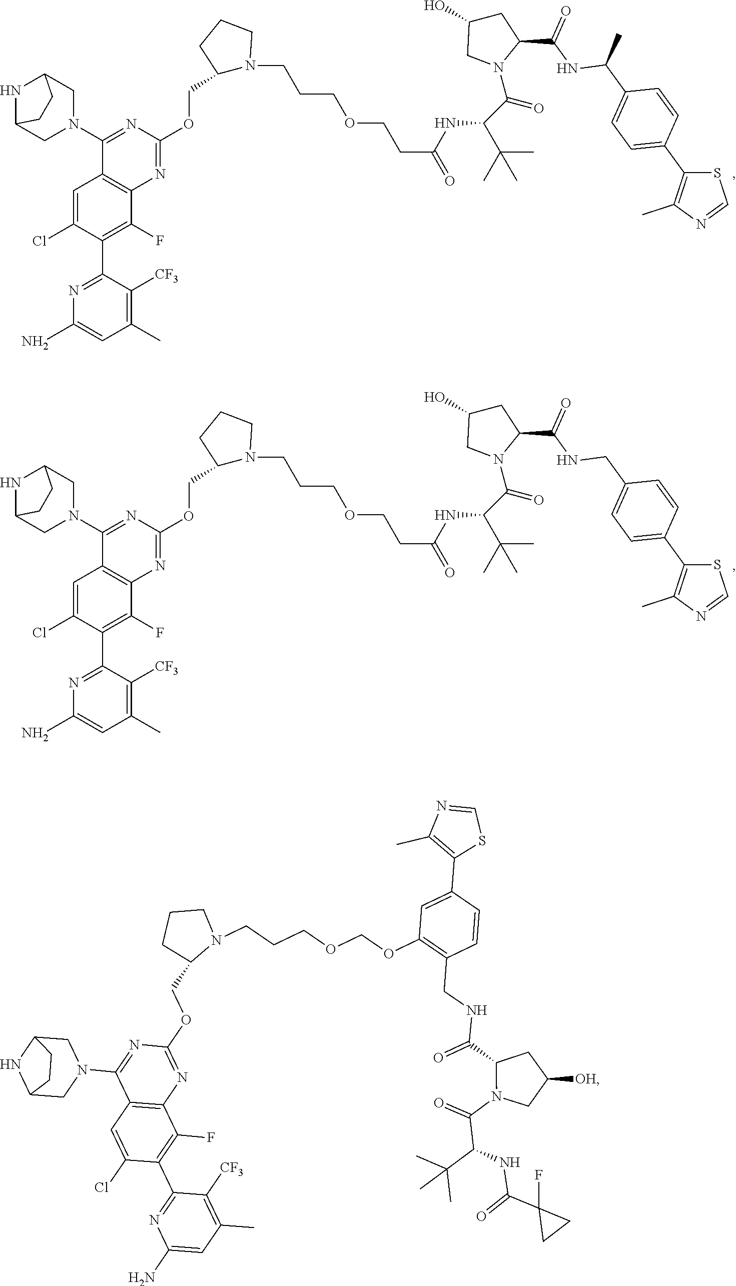 [SMILES: Cc1cc(N)nc(-c2c(Cl)cc3c(N4CC5CCC(C4)N5)nc(OC[C@@H]4CCCN4CCCOCCC(=O)N[C@H](C(=O)N4C[C@H](O)C[C@H]4C(=O)N[C@@H](C)c4ccc(-c5scnc5C)cc4)C(C)(C)C)nc3c2F)c1C(F)(F)F ; Cc1cc(N)nc(-c2c(Cl)cc3c(N4CC5CCC(C4)N5)nc(OC[C@@H]4CCCN4CCCOCCC(=O)N[C@H](C(=O)N4C[C@H](O)C[C@H]4C(=O)NCc4ccc(-c5scnc5C)cc4)C(C)(C)C)nc3c2F)c1C(F)(F)F ; Cc1cc(N)nc(-c2c(Cl)cc3c(N4CC5CCC(C4)N5)nc(OC[C@@H]4CCCN4CCCOCOc4cc(-c5scnc5C)ccc4CNC(=O)[C@@H]4C[C@@H](O)CN4C(=O)[C@H](NC(=O)C4(F)CC4)C(C)(C)C)nc3c2F)c1C(F)(F)F]

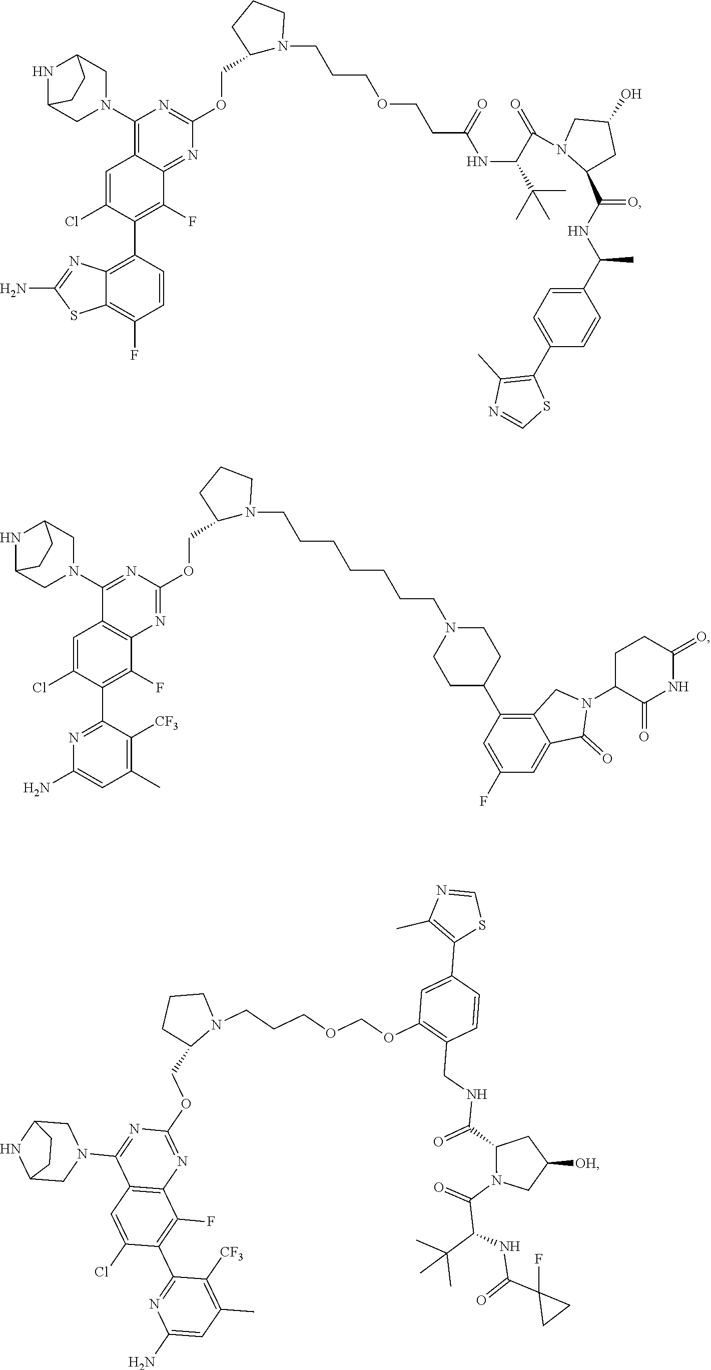 [SMILES: Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cc(Cl)c(-c4ccc(F)c5sc(N)nc45)c(F)c3n2)C(C)(C)C)cc1 ; Cc1cc(N)nc(-c2c(Cl)cc3c(N4CC5CCC(C4)N5)nc(OC[C@@H]4CCCN4CCCCCCCN4CCC(c5cc(F)cc6c5CN(C5CCC(=O)NC5=O)C6=O)CC4)nc3c2F)c1C(F)(F)F ; Cc1cc(N)nc(-c2c(Cl)cc3c(N4CC5CCC(C4)N5)nc(OC[C@@H]4CCCN4CCCOCOc4cc(-c5scnc5C)ccc4CNC(=O)[C@@H]4C[C@@H](O)CN4C(=O)[C@H](NC(=O)C4(F)CC4)C(C)(C)C)nc3c2F)c1C(F)(F)F]

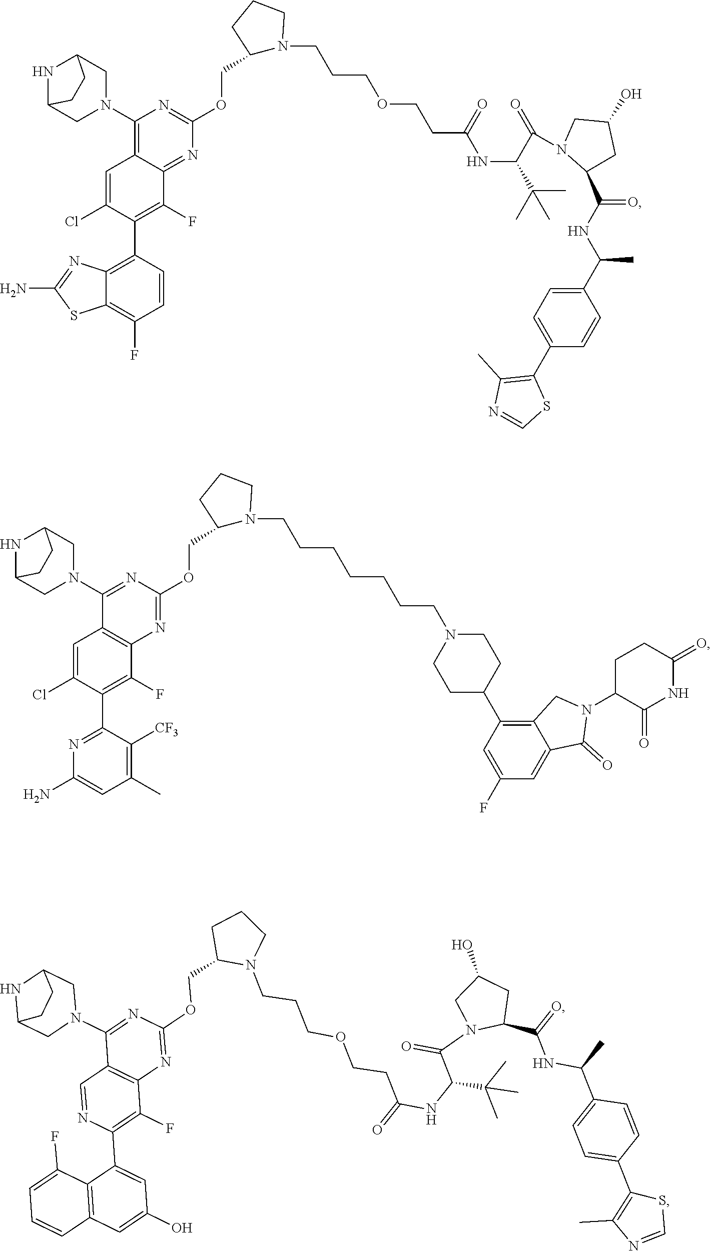 [SMILES: Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cc(Cl)c(-c4ccc(F)c5sc(N)nc45)c(F)c3n2)C(C)(C)C)cc1 ; Cc1cc(N)nc(-c2c(Cl)cc3c(N4CC5CCC(C4)N5)nc(OC[C@@H]4CCCN4CCCCCCCN4CCC(c5cc(F)cc6c5CN(C5CCC(=O)NC5=O)C6=O)CC4)nc3c2F)c1C(F)(F)F ; Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cnc(-c4cc(O)cc5cccc(F)c45)c(F)c3n2)C(C)(C)C)cc1]

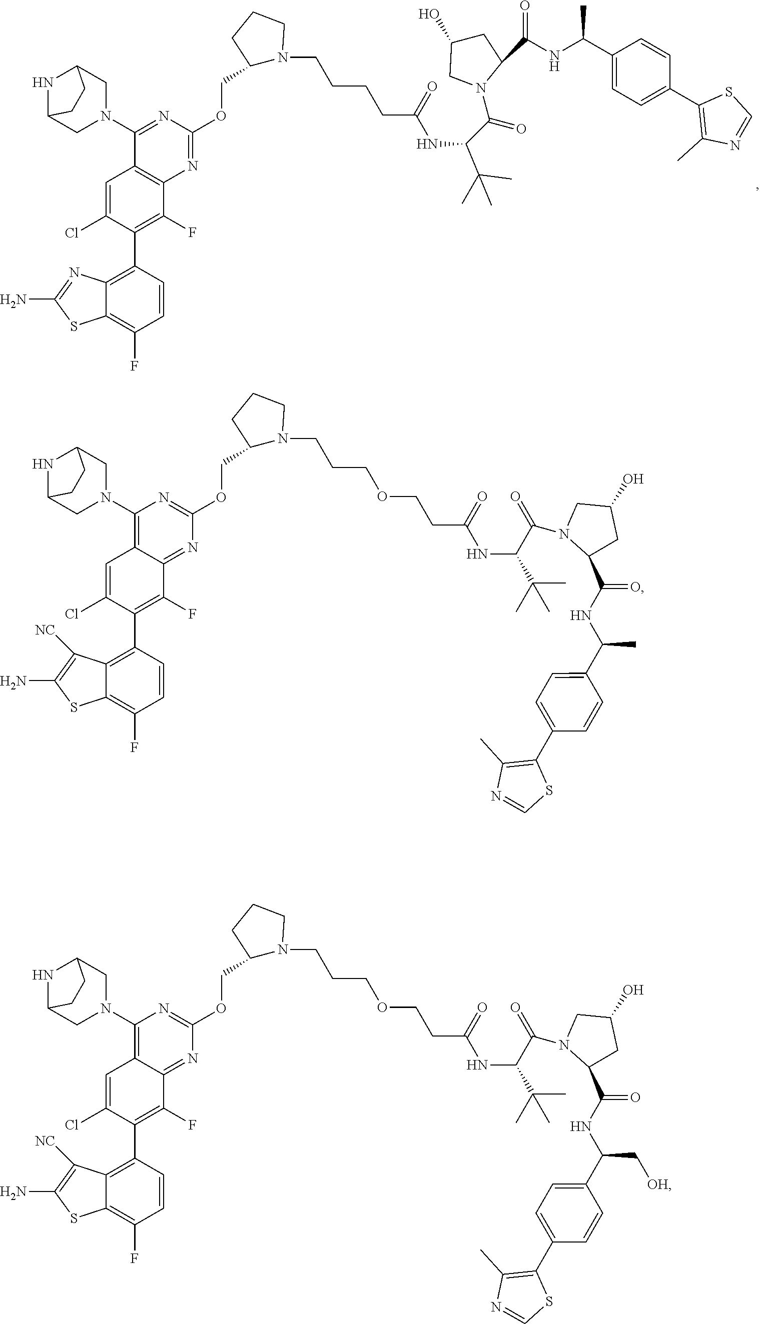 [SMILES: Cc1ncsc1-c1ccc([C@H](C)NC(=O)[C@@H]2C[C@@H](O)CN2C(=O)[C@@H](NC(=O)CCCCN2CCC[C@H]2COc2nc(N3CC4CCC(C3)N4)c3cc(Cl)c(-c4ccc(F)c5sc(N)nc45)c(F)c3n2)C(C)(C)C)cc1 ; Cc1ncsc1-c1ccc([C@H](C)NC(=O)[C@@H]2C[C@@H](O)CN2C(=O)[C@@H](NC(=O)CCOCCCN2CCC[C@H]2COc2nc(N3CC4CCC(C3)N4)c3cc(Cl)c(-c4ccc(F)c5sc(N)c(C#N)c45)c(F)c3n2)C(C)(C)C)cc1 ; Cc1ncsc1-c1ccc([C@H](CO)NC(=O)[C@@H]2C[C@@H](O)CN2C(=O)[C@@H](NC(=O)CCOCCCN2CCC[C@H]2COc2nc(N3CC4CCC(C3)N4)c3cc(Cl)c(-c4ccc(F)c5sc(N)c(C#N)c45)c(F)c3n2)C(C)(C)C)cc1]

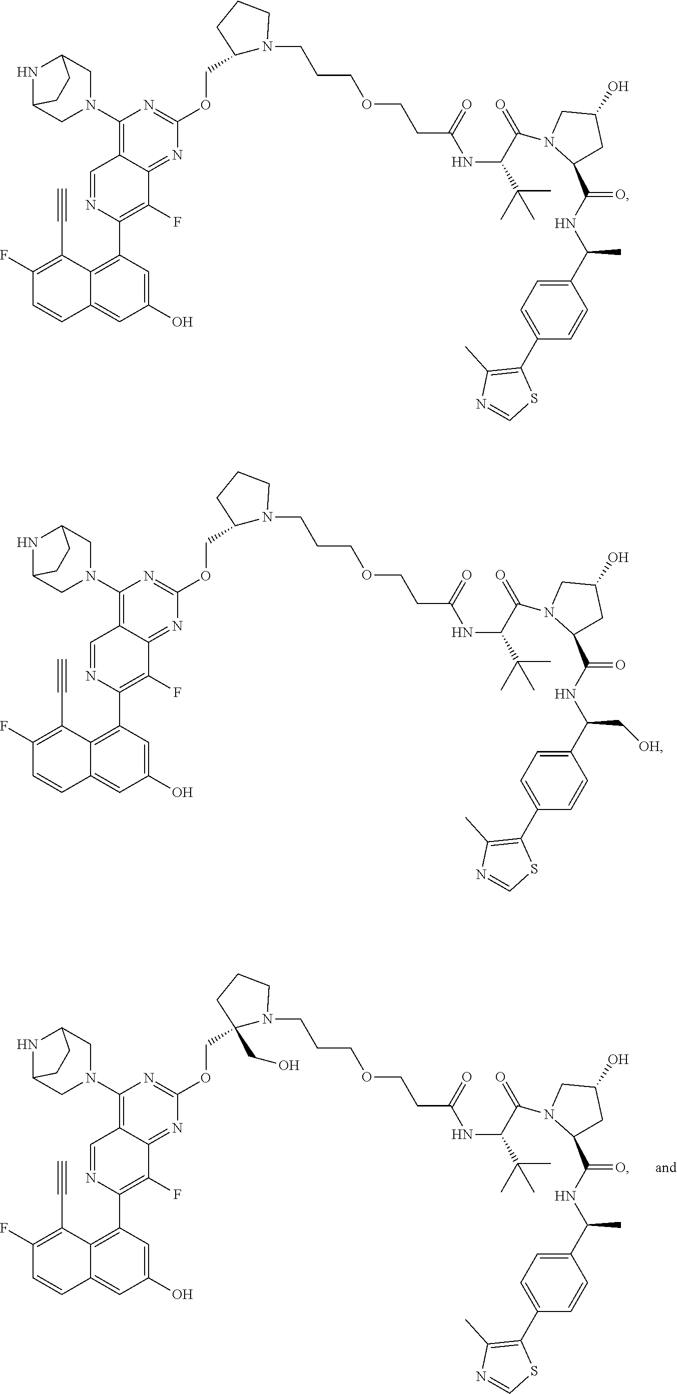 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6scnc6C)cc5)C(C)(C)C)nc4c3F)c12 ; C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](CO)c5ccc(-c6scnc6C)cc5)C(C)(C)C)nc4c3F)c12 ; C#Cc1c(F)ccc2cc(O)cc(-c3ncc4c(N5CC6CCC(C5)N6)nc(OC[C@]5(CO)CCCN5CCCOCCC(=O)N[C@H](C(=O)N5C[C@H](O)C[C@H]5C(=O)N[C@@H](C)c5ccc(-c6scnc6C)cc5)C(C)(C)C)nc4c3F)c12]

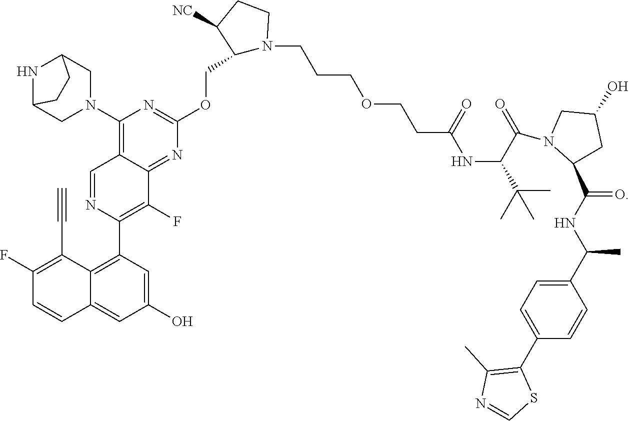 [SMILES: C#Cc1c(F)ccc2cc(O)cc(-c3ncc4c(N5CC6CCC(C5)N6)nc(OC[C@@H]5[C@@H](C#N)CCN5CCCOCCC(=O)N[C@H](C(=O)N5C[C@H](O)C[C@H]5C(=O)N[C@@H](C)c5ccc(-c6scnc6C)cc5)C(C)(C)C)nc4c3F)c12]

27. The conjugate of any one of claims 1 to 24, wherein the conjugate is selected from:

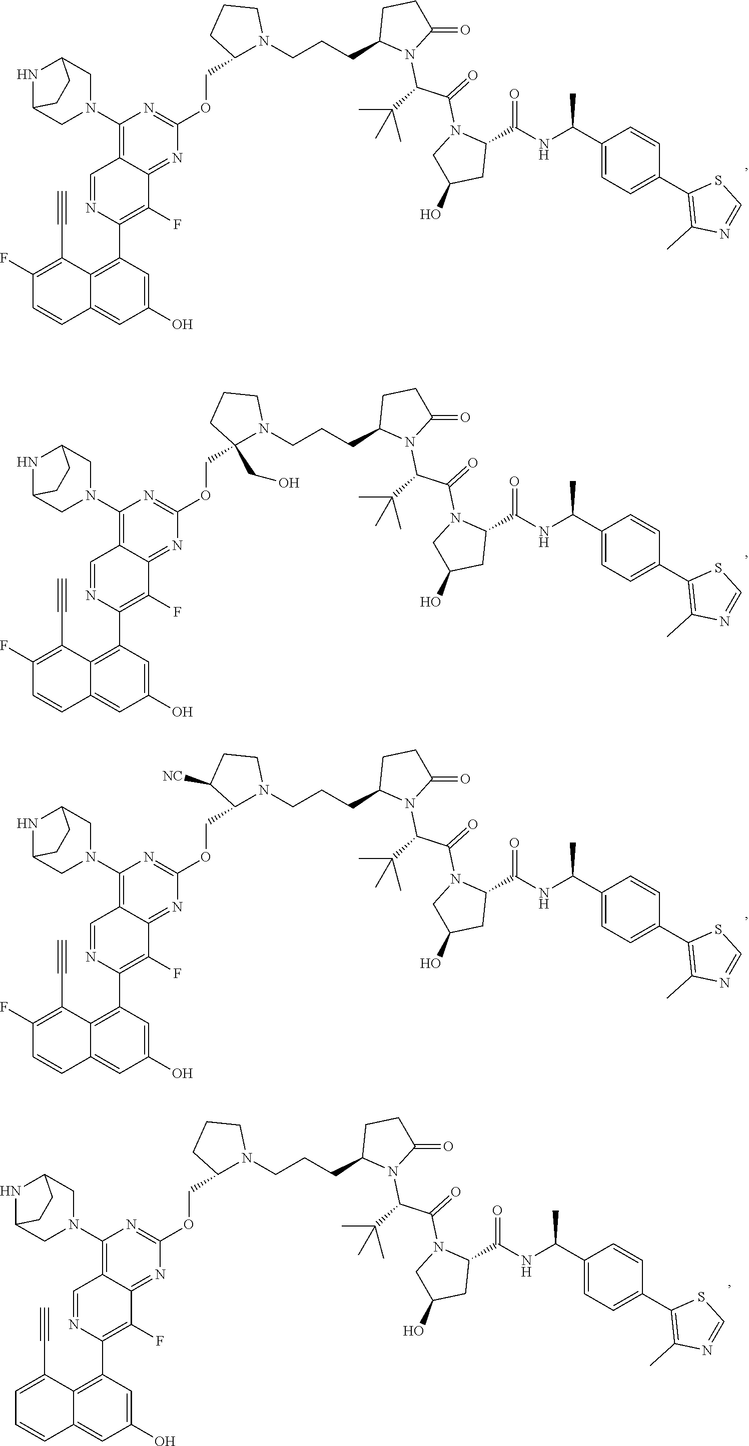

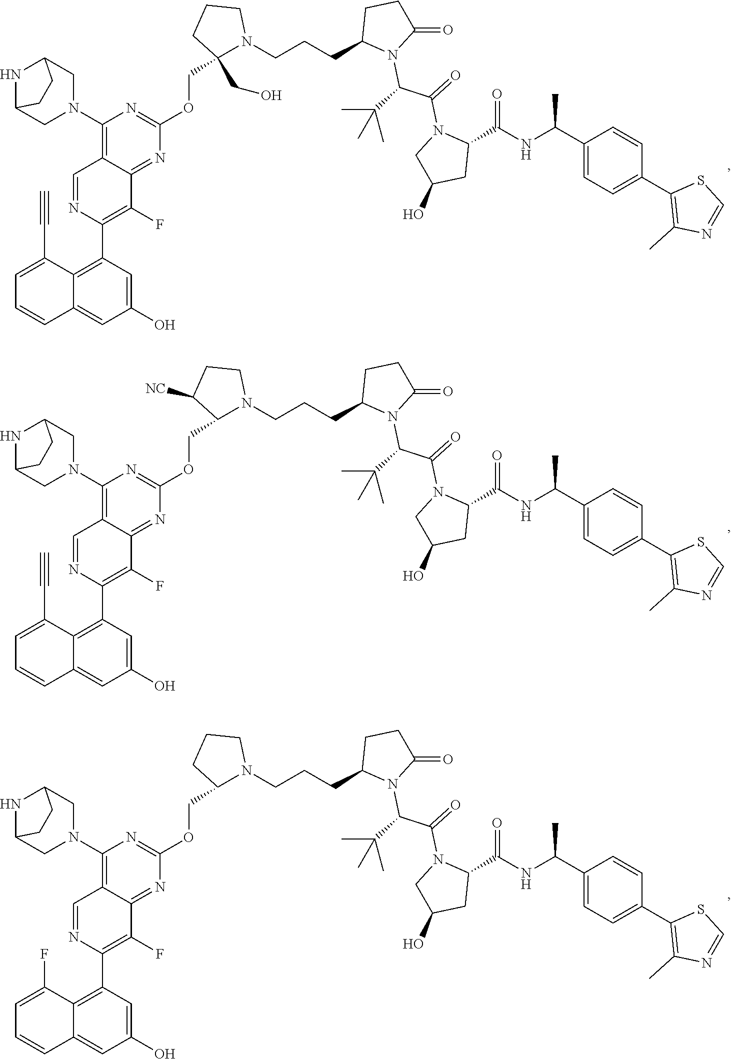 [SMILES: C#Cc1cccc2cc(O)cc(-c3ncc4c(N5CC6CCC(C5)N6)nc(OC[C@]5(CO)CCCN5CCC[C@H]5CCC(=O)N5[C@H](C(=O)N5C[C@H](O)C[C@H]5C(=O)N[C@@H](C)c5ccc(-c6scnc6C)cc5)C(C)(C)C)nc4c3F)c12 ; C#Cc1cccc2cc(O)cc(-c3ncc4c(N5CC6CCC(C5)N6)nc(OC[C@@H]5[C@@H](C#N)CCN5CCC[C@H]5CCC(=O)N5[C@H](C(=O)N5C[C@H](O)C[C@H]5C(=O)N[C@@H](C)c5ccc(-c6scnc6C)cc5)C(C)(C)C)nc4c3F)c12 ; Cc1ncsc1-c1ccc([C@H](C)NC(=O)[C@@H]2C[C@@H](O)CN2C(=O)[C@@H](N2C(=O)CC[C@@H]2CCCN2CCC[C@H]2COc2nc(N3CC4CCC(C3)N4)c3cnc(-c4cc(O)cc5cccc(F)c45)c(F)c3n2)C(C)(C)C)cc1]

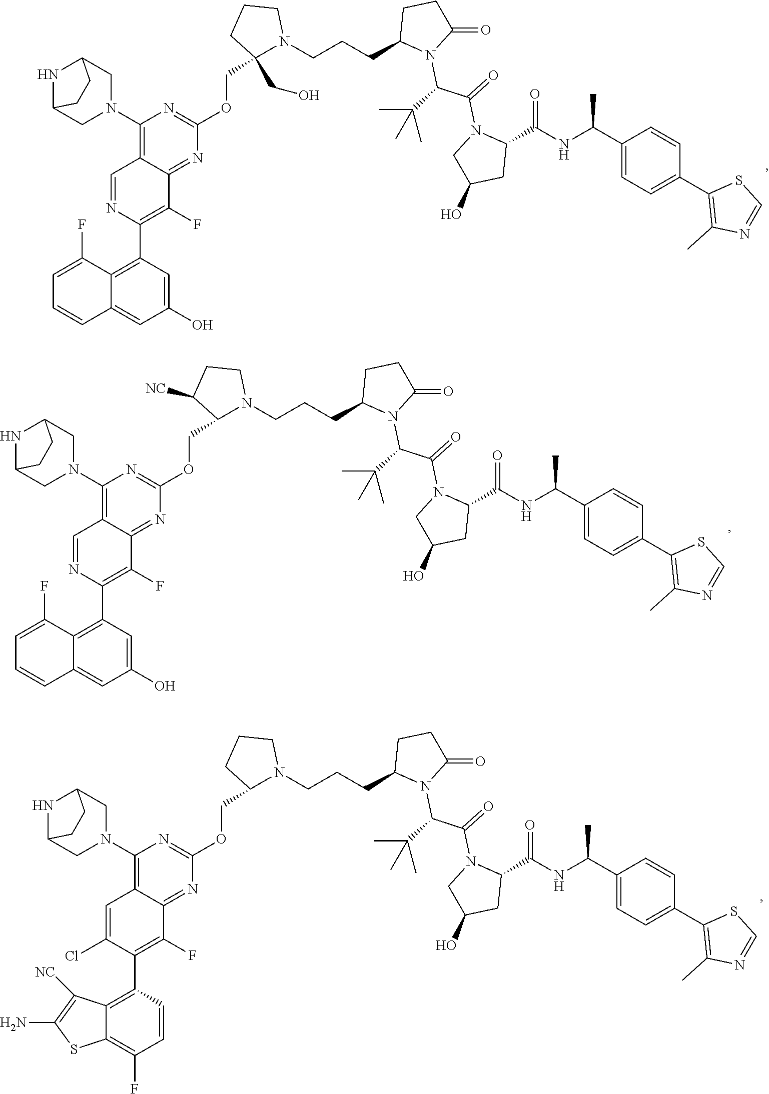

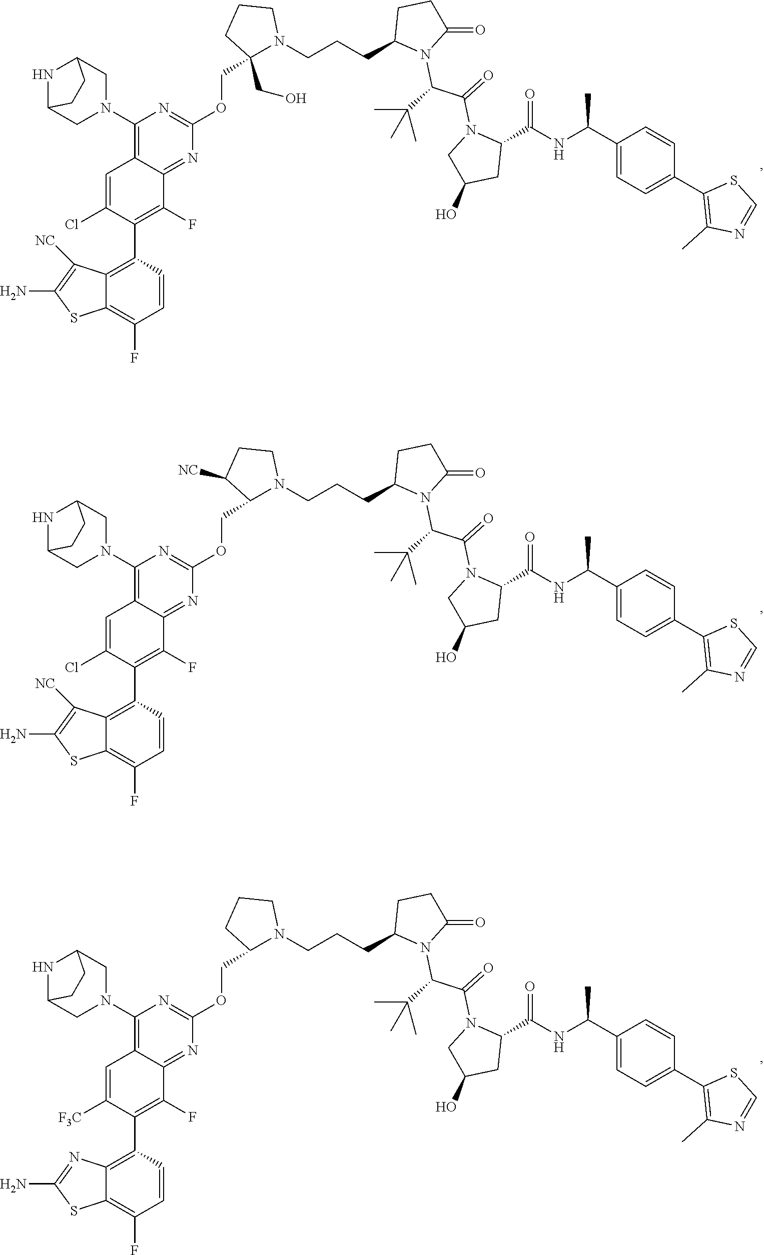

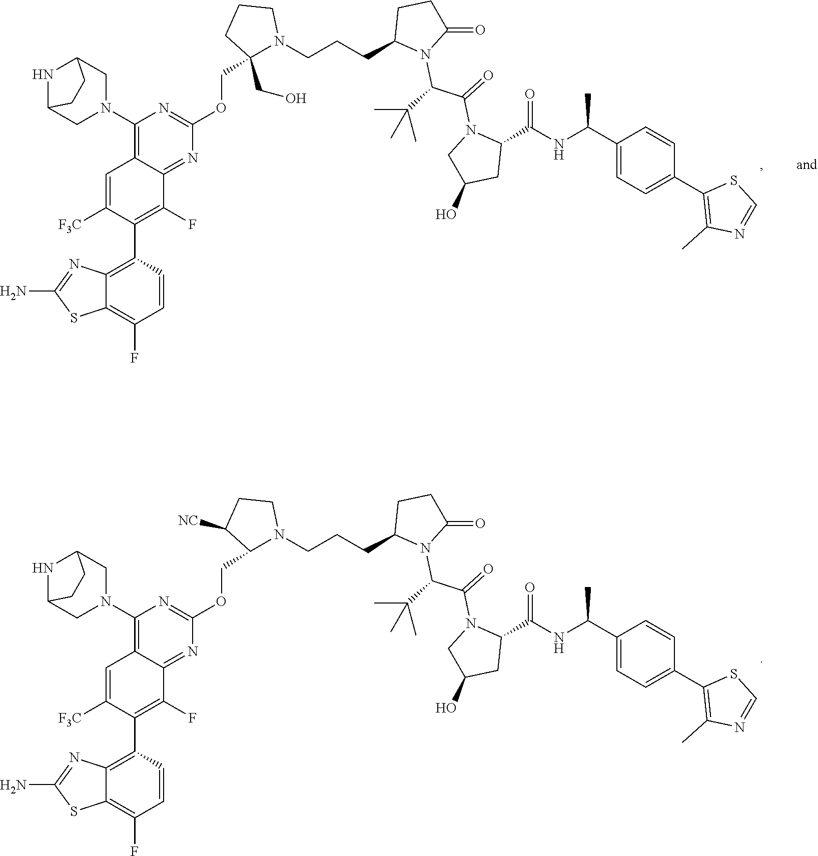

28. The conjugate of any one of claims 1 to 24, wherein the conjugate is selected from:

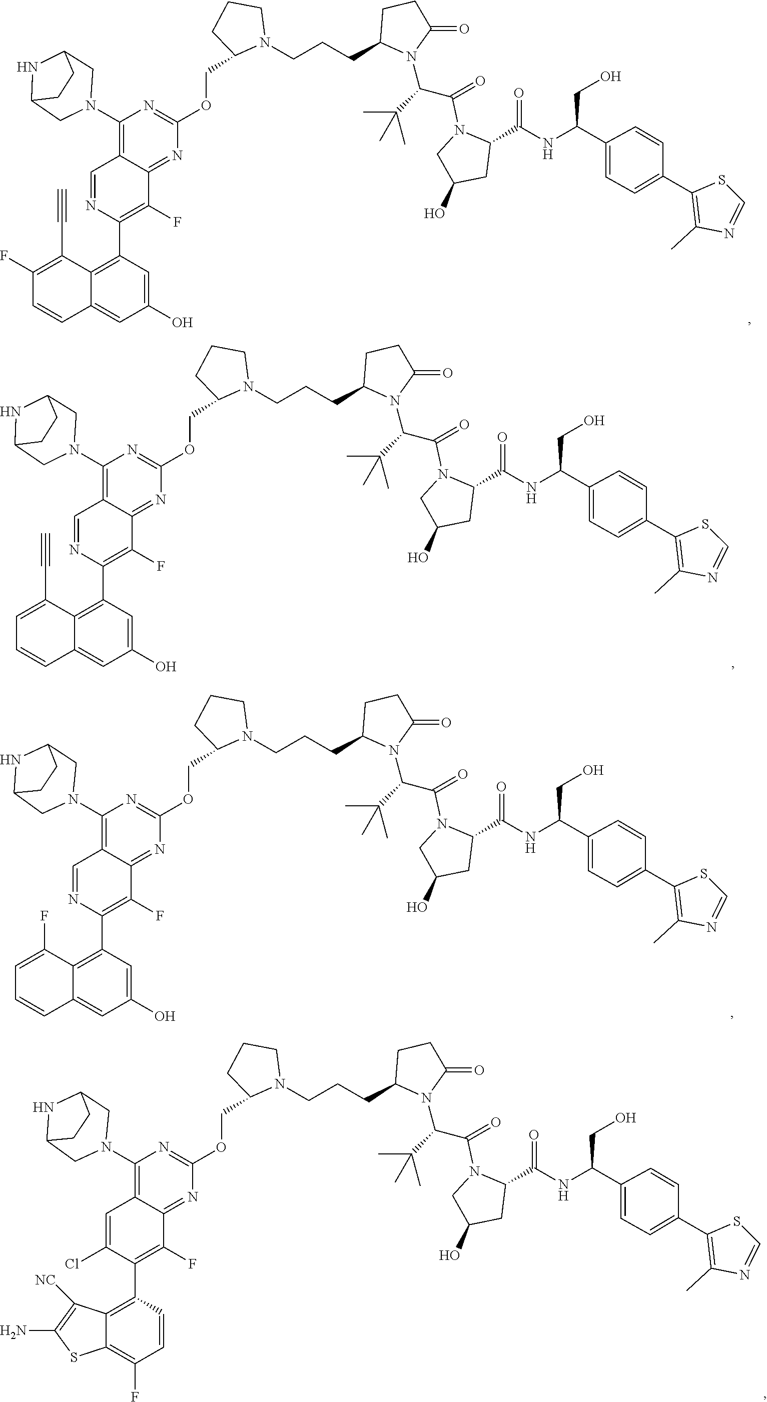

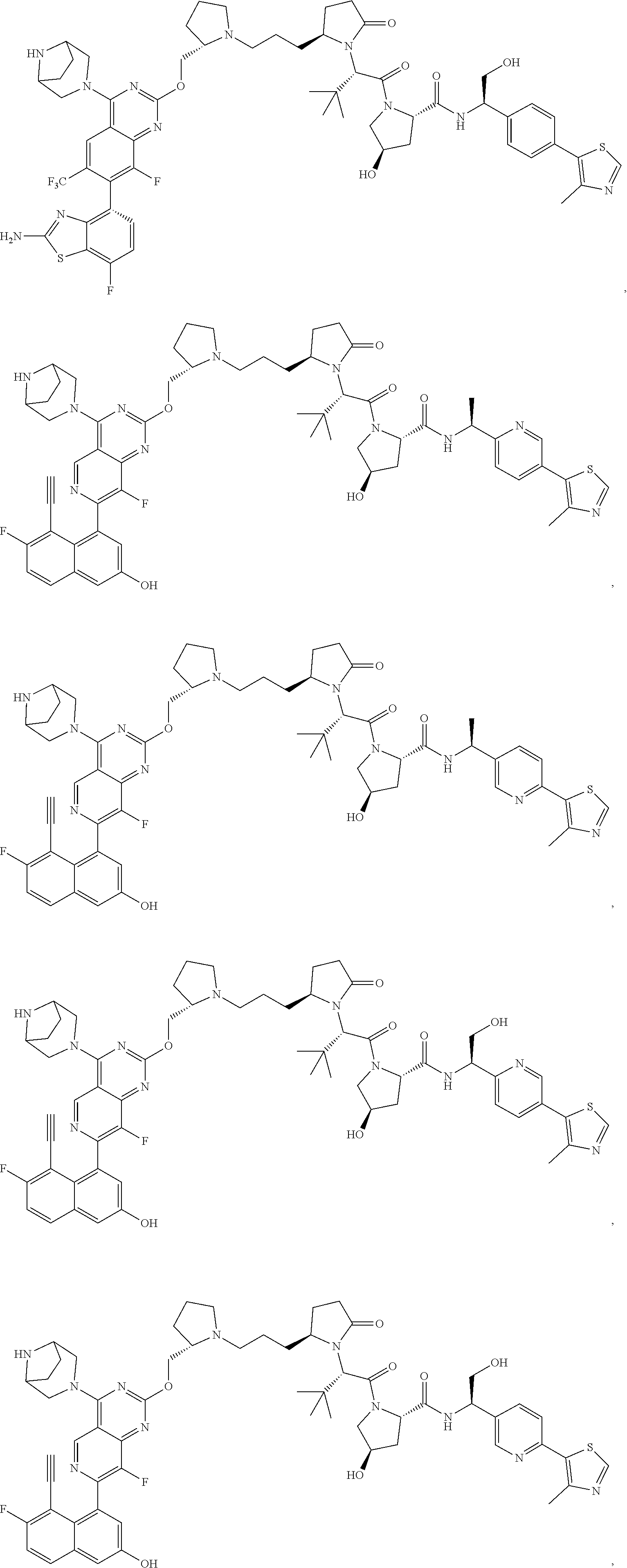

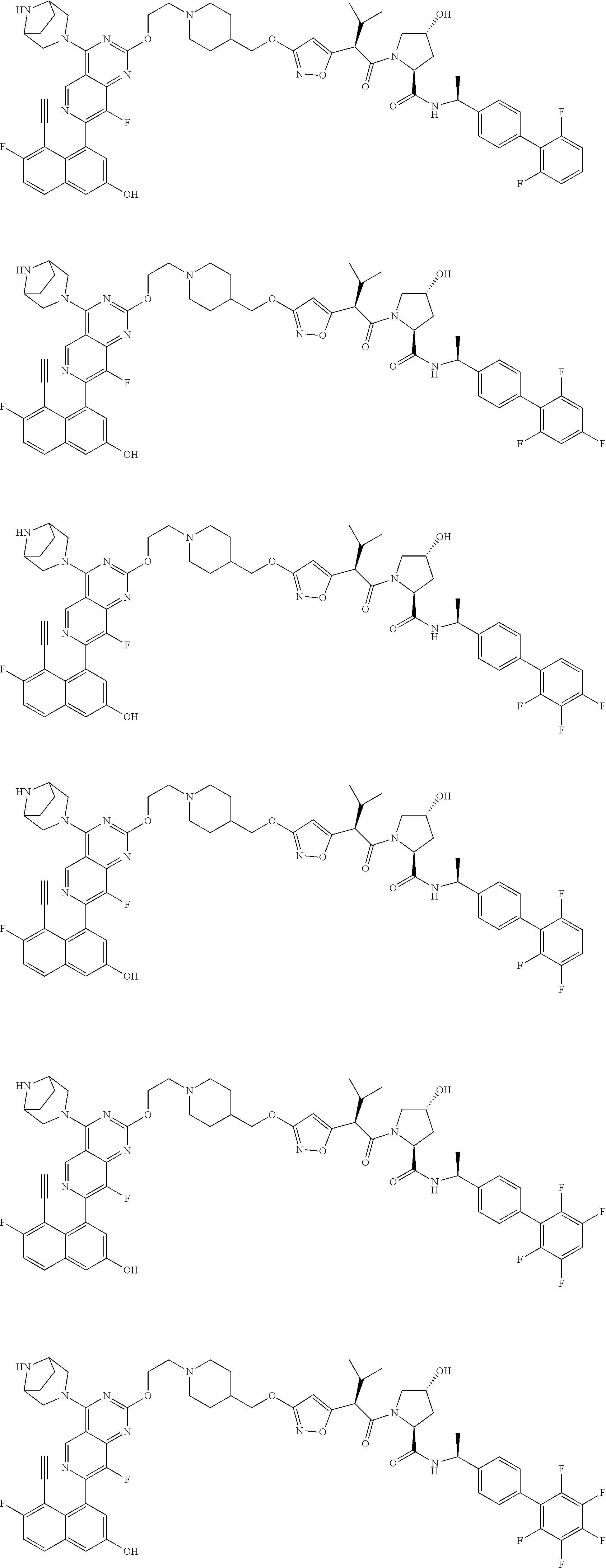

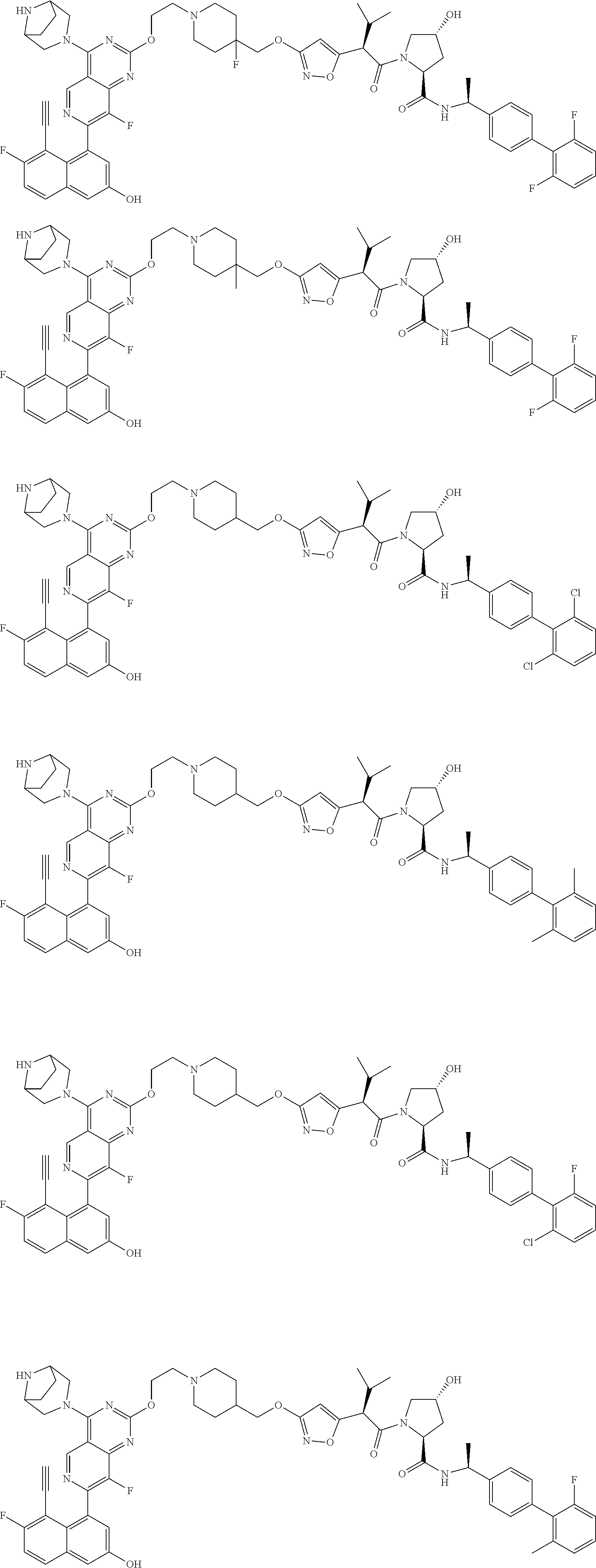

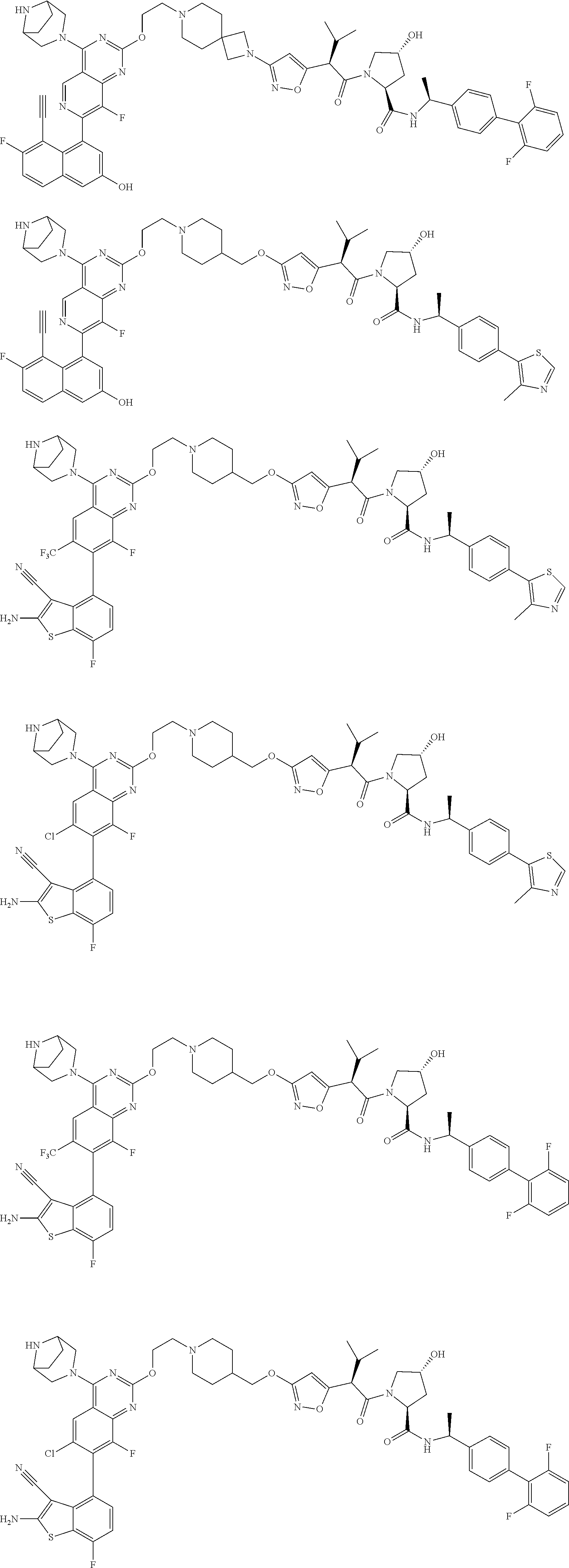

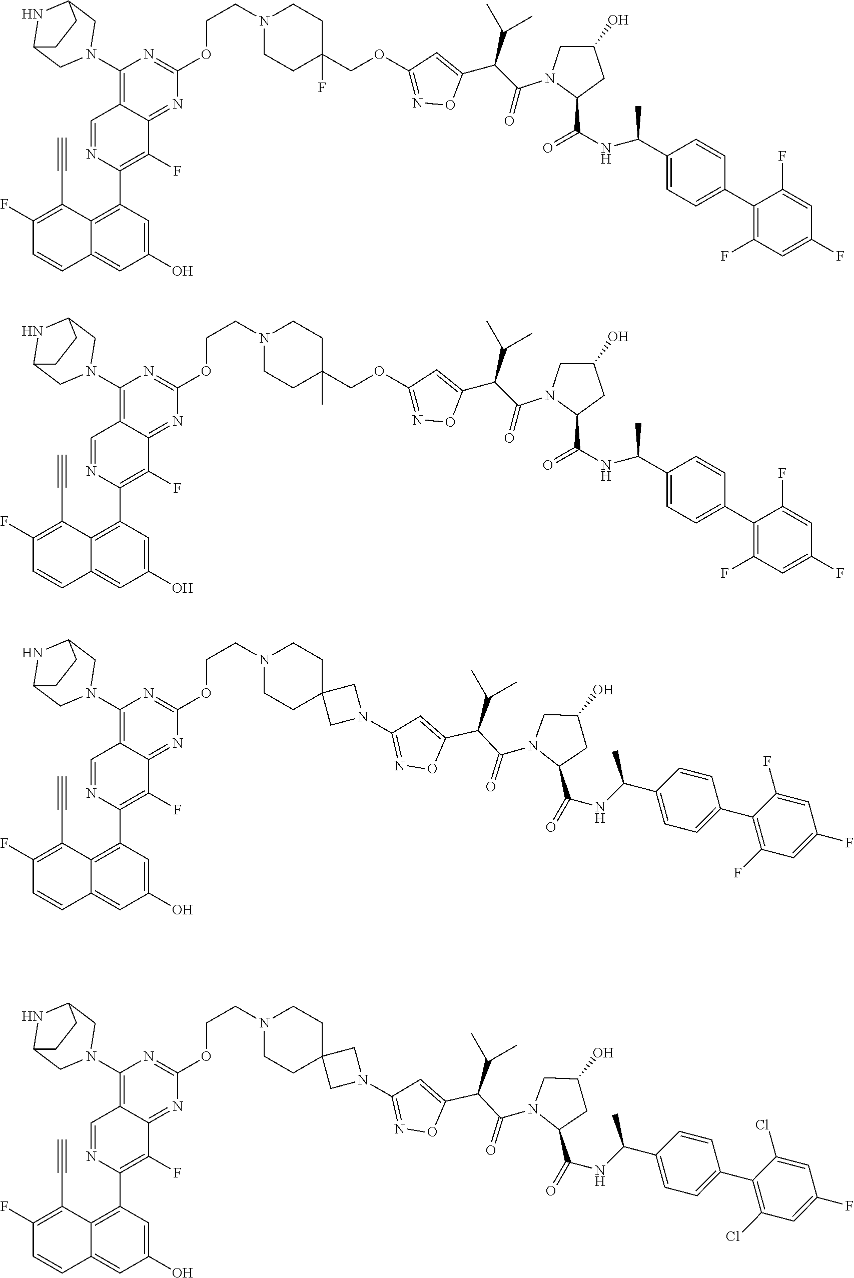

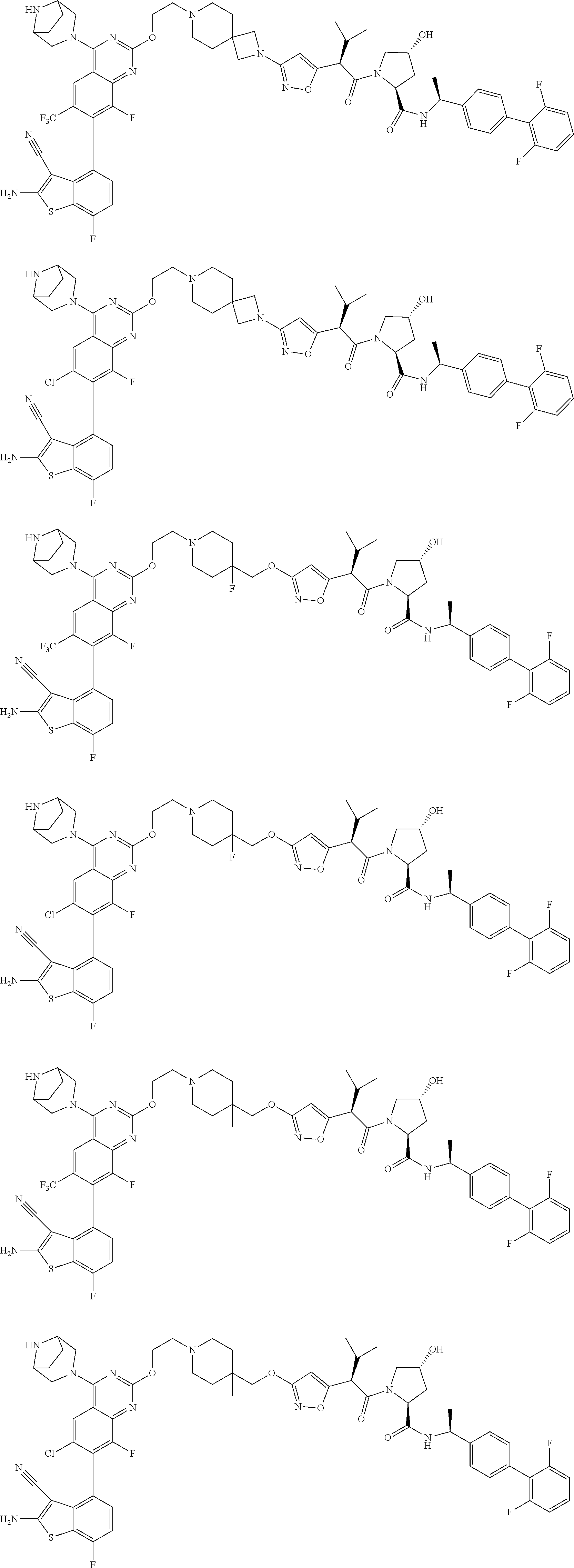

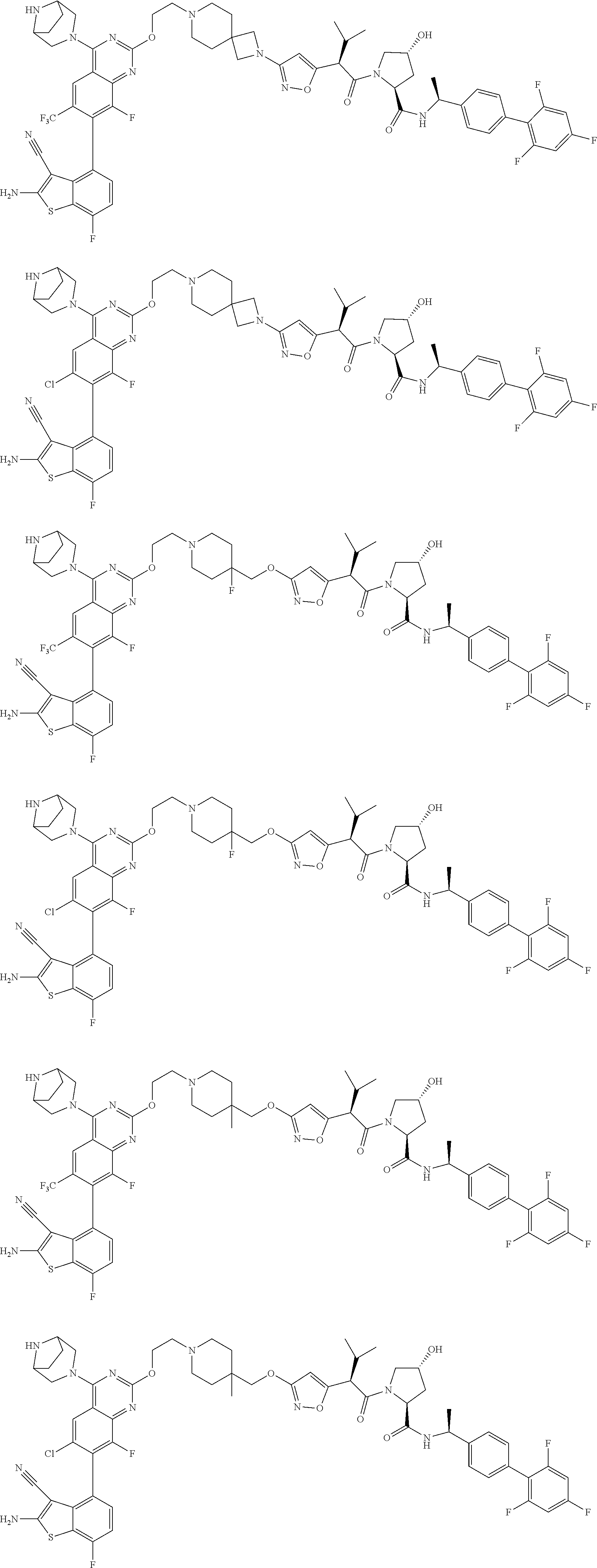

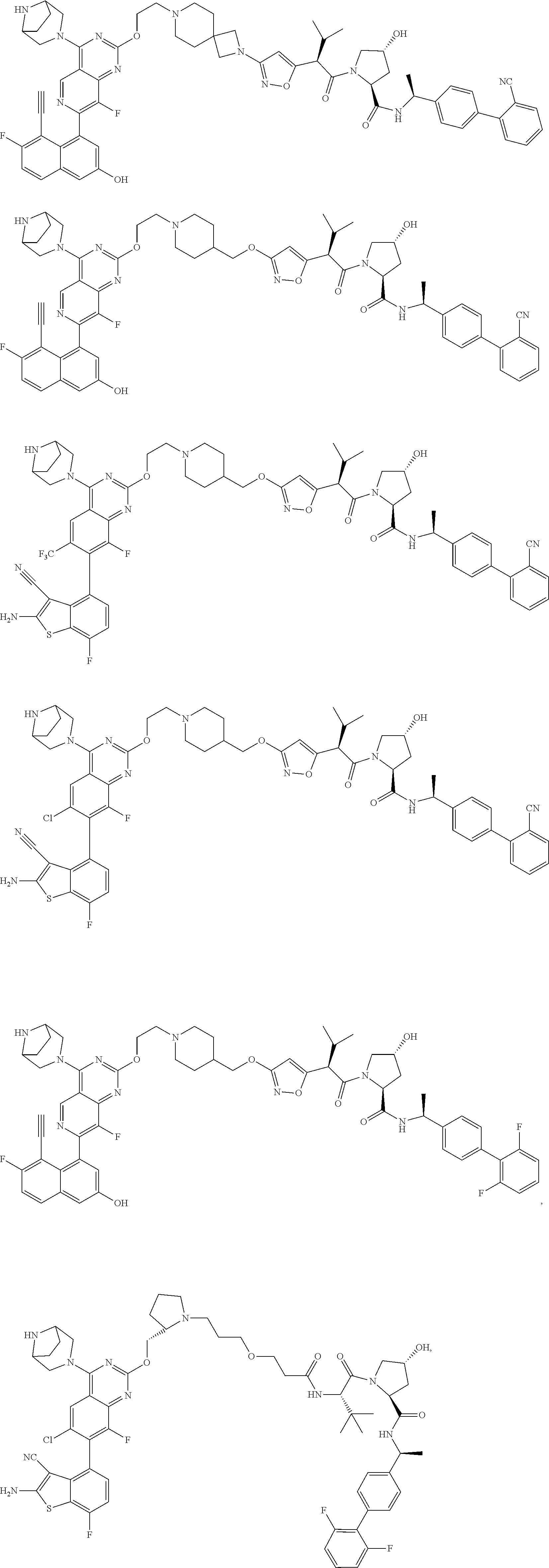

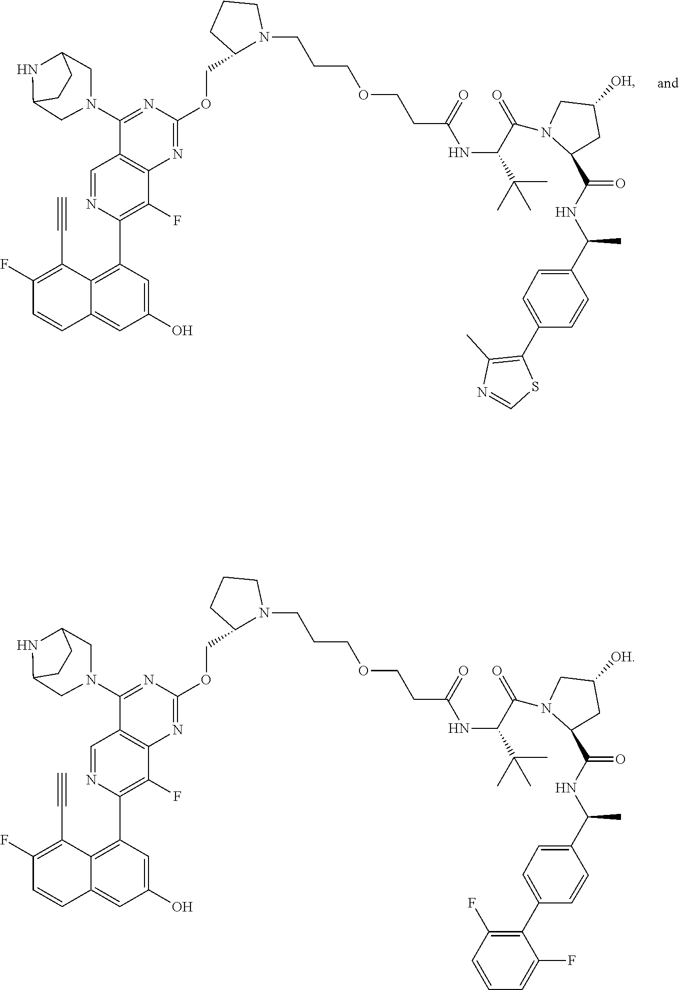 [SMILES: C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6scnc6C)cc5)C(C)(C)C)nc4c3F)c12 ; C#Cc1c(F)ccc2cc(O)cc(-c3ncc4c(N5CC6CCC(C5)N6)nc(OC[C@@H]5CCCN5CCCOCCC(=O)N[C@H](C(=O)N5C[C@H](O)C[C@H]5C(=O)N[C@@H](C)c5ccc(-c6c(F)cccc6F)cc5)C(C)(C)C)nc4c3F)c12]

29. The conjugate of any one of claims 1 to 24, wherein the conjugate is selected from:

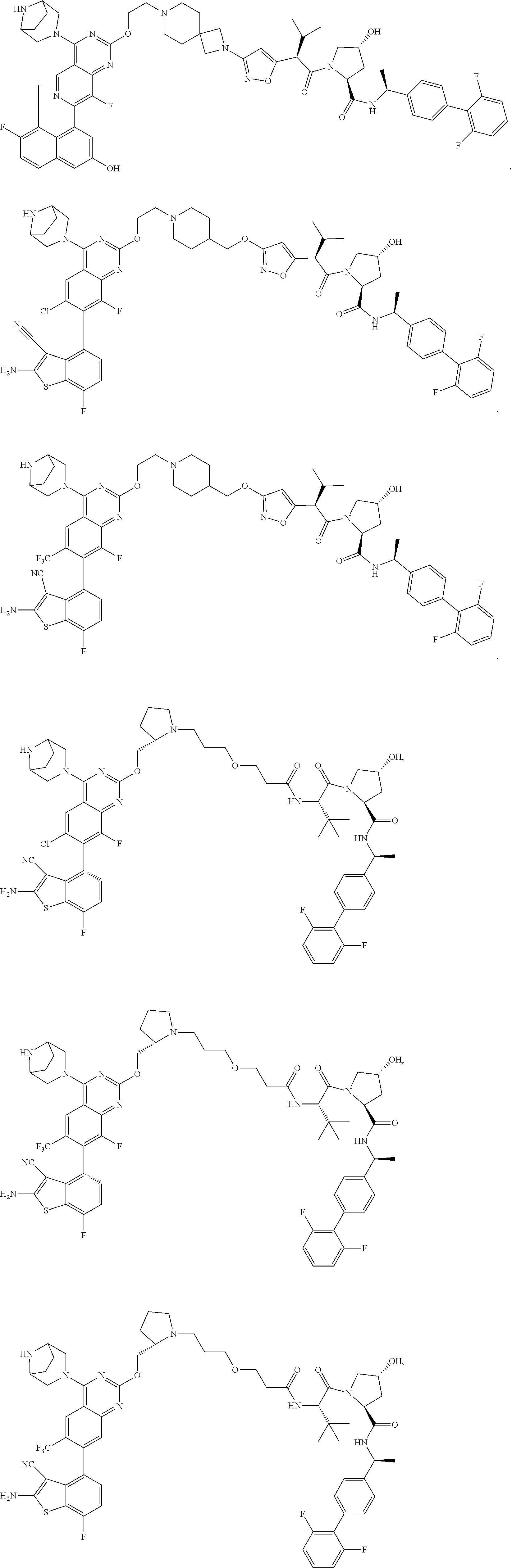

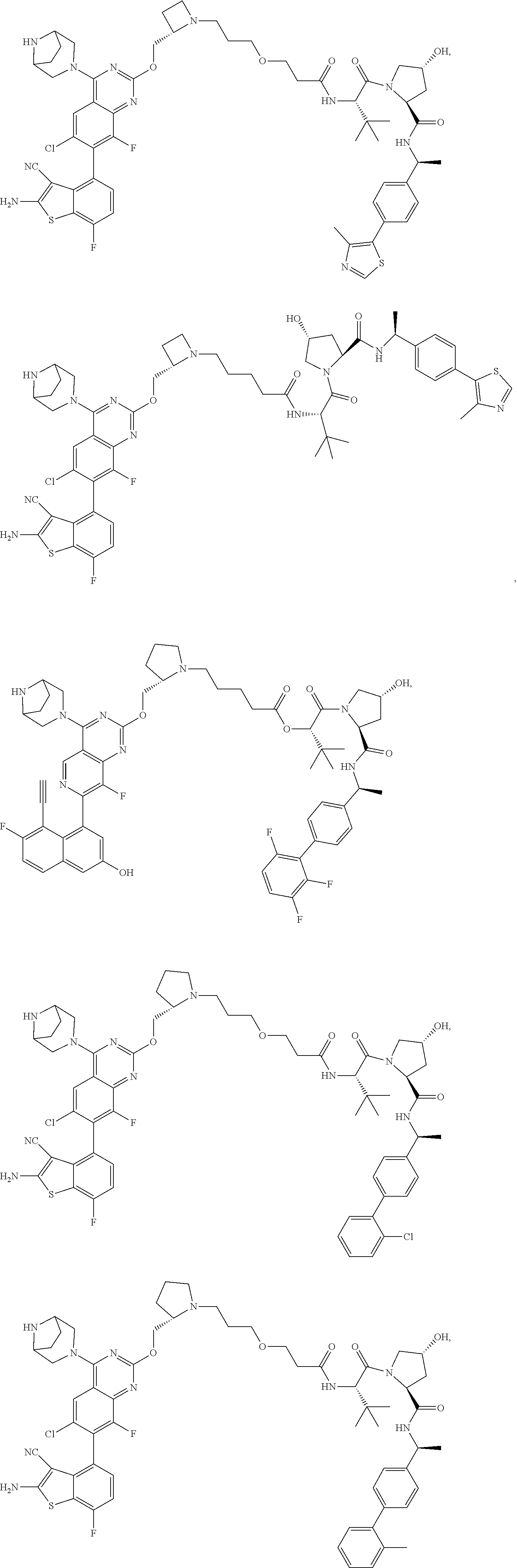

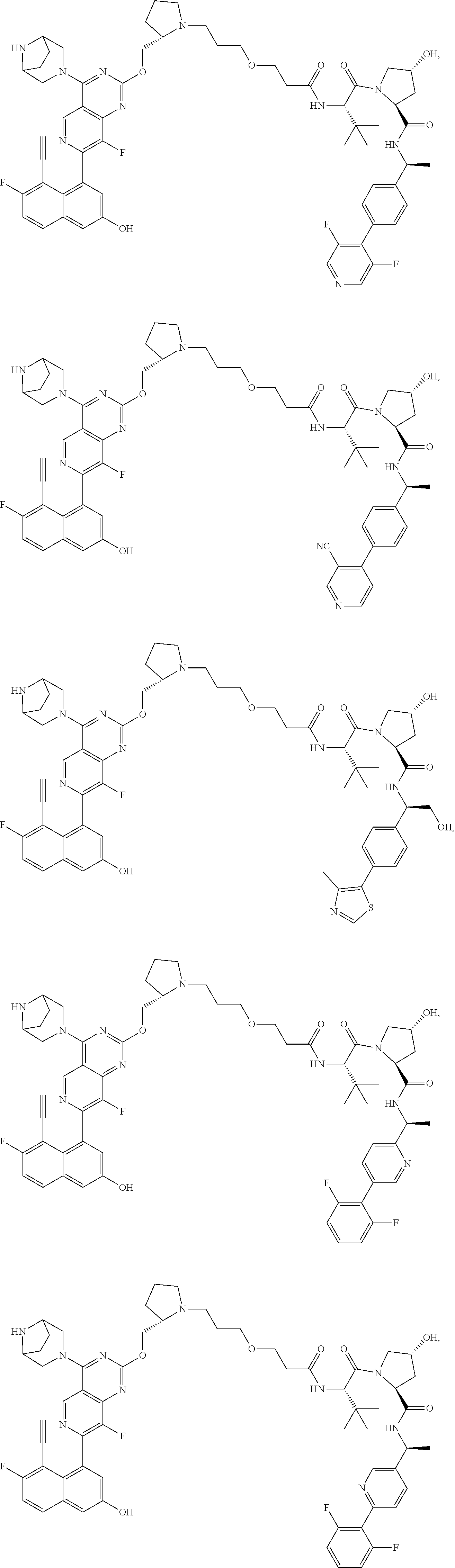

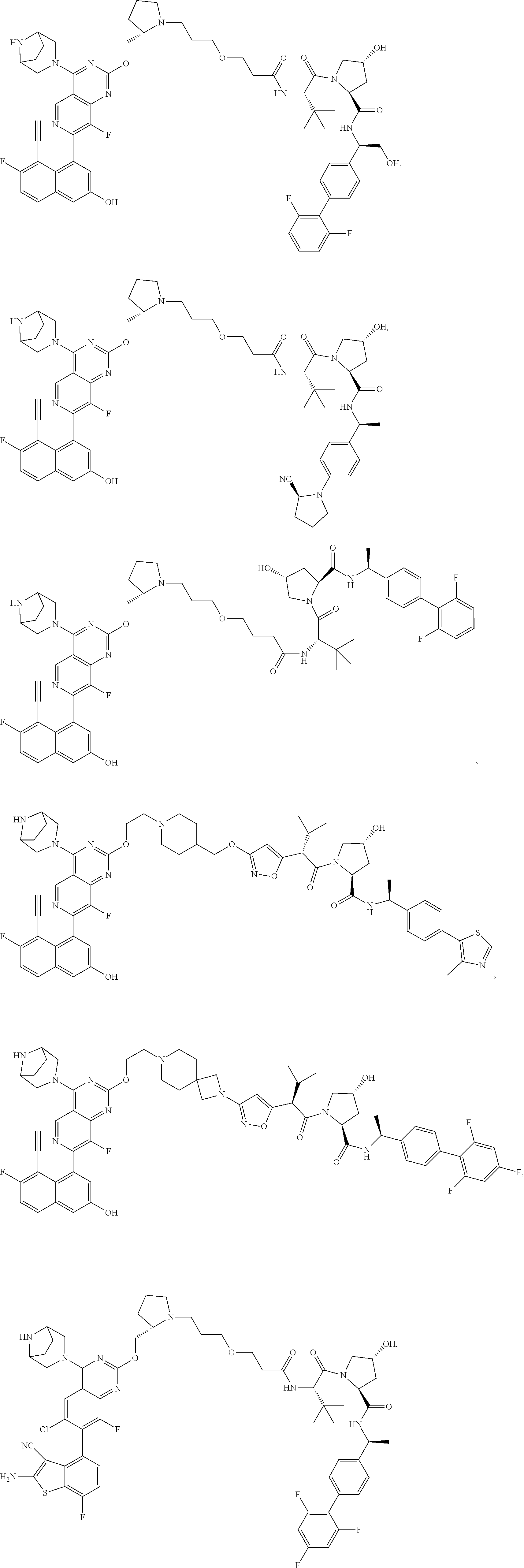

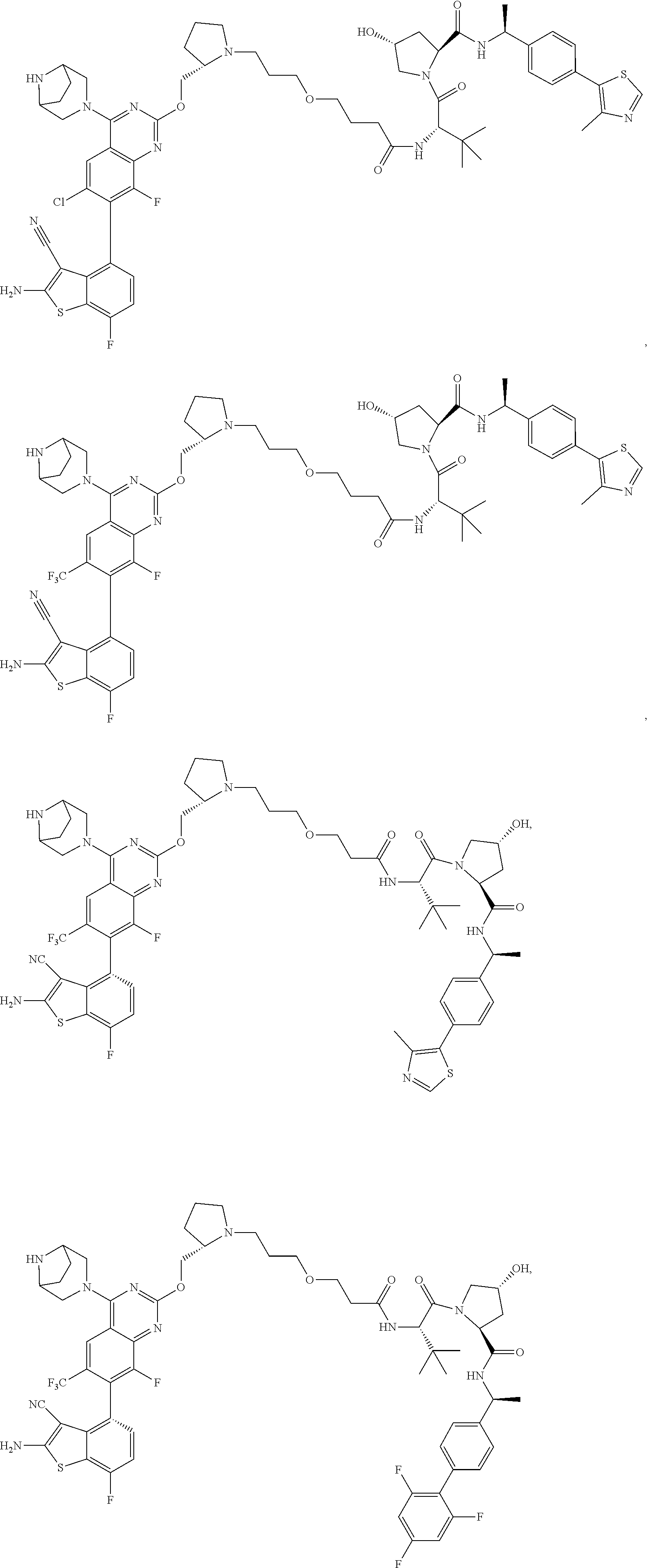

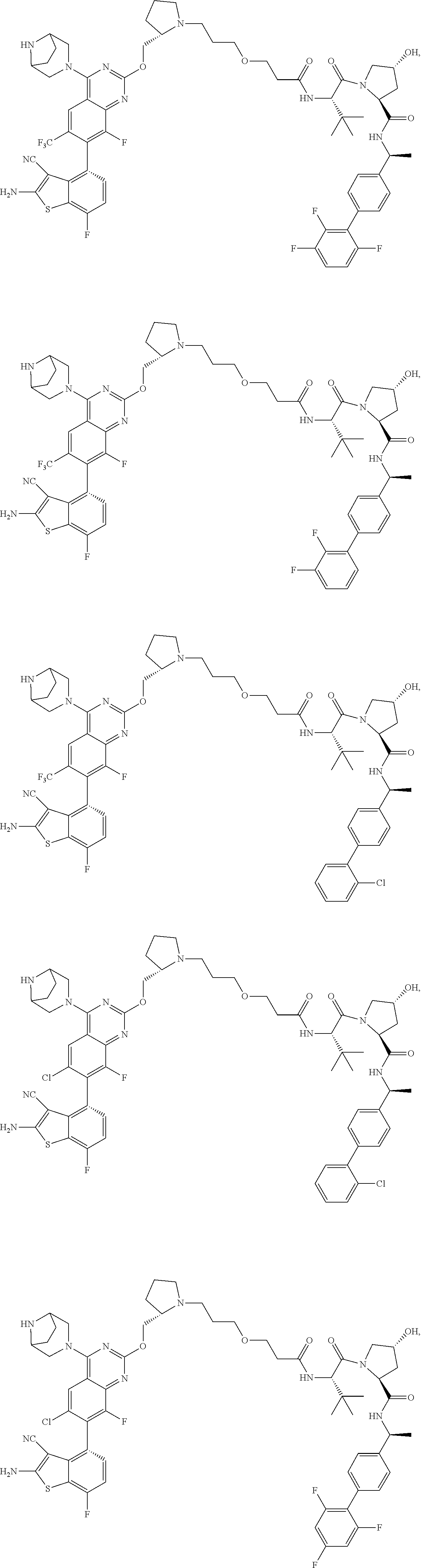

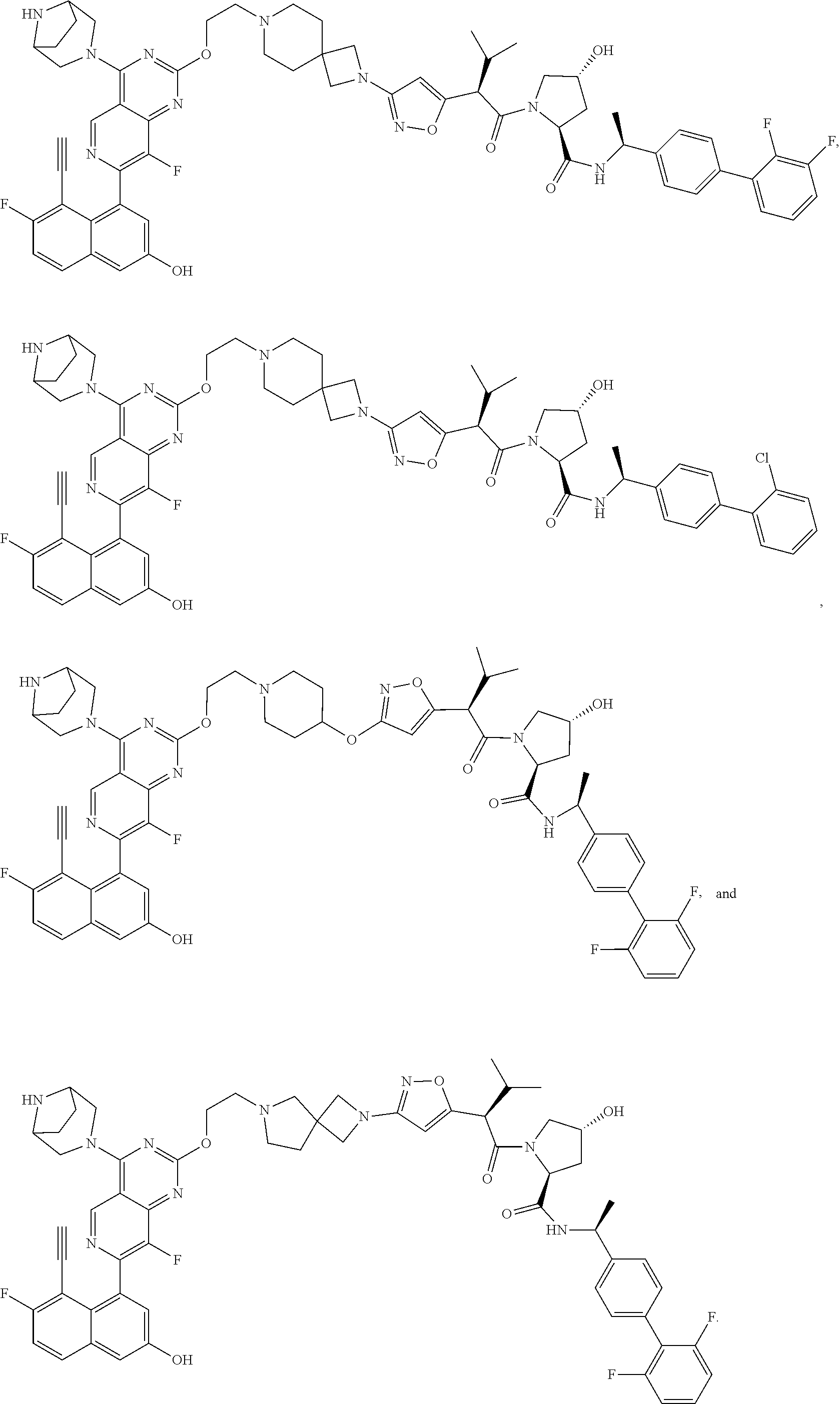

30. A pharmaceutical composition comprising a pharmaceutically effective amount of a conjugate of any one of claims 1 to 29, or a pharmaceutically acceptable salt thereof, and a pharmaceutically acceptable excipient.

31. The pharmaceutical composition of claim 30, further comprising an additional therapeutic agent.

32. A method of treating a subject having cancer, the cancer characterized by the presence of a KRAS G12D mutation, the method comprising administering to the subject a therapeutically effective amount of a conjugate of any one of claims 1 to 29, or a pharmaceutically acceptable salt thereof, or a pharmaceutical composition thereof.

33. The method of claim 32, wherein the cancer is Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; or Adrenal glands: neuroblastoma.

34. The method of claim 32, wherein the cancer is non-small cell lung cancer, small cell lung cancer, colorectal cancer, rectal cancer, or pancreatic cancer.

35. Use of a conjugate of any one of claims 1 to 29, or a pharmaceutically acceptable salt thereof, or a a pharmaceutical composition thereof, in the manufacture of a medicament for the treatment of cancer in a subject, the cancer characterized by the presence of a KRAS G12D mutation.

36. The use of claim 35, wherein the cancer is Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial 'carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; or Adrenal glands: neuroblastoma.

37. The method of claim 35, wherein the cancer is non-small cell lung cancer, small cell lung cancer, colorectal cancer, rectal cancer, or pancreatic cancer.

38. A conjugate of Formula P, or pharmaceutically acceptable salt thereof: 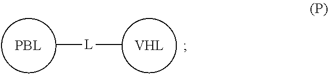
wherein PBL is a protein binding ligand capable of binding and/or inhibiting to a target protein; L is a bivalent linker that connects PBL to a Von Hippel-Lindau (VHL) moiety; wherein VHL binds to a ubiquitin ligase; and
wherein the VHL ligand is: 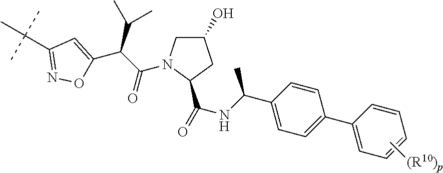
wherein p is an integer from 0 to 5; and
each R¹⁰ is independently selected from alkyl, cyano, and halogen.

39. The conjugate of claim 38, wherein the VHL ligand is selected from the group consisting of:

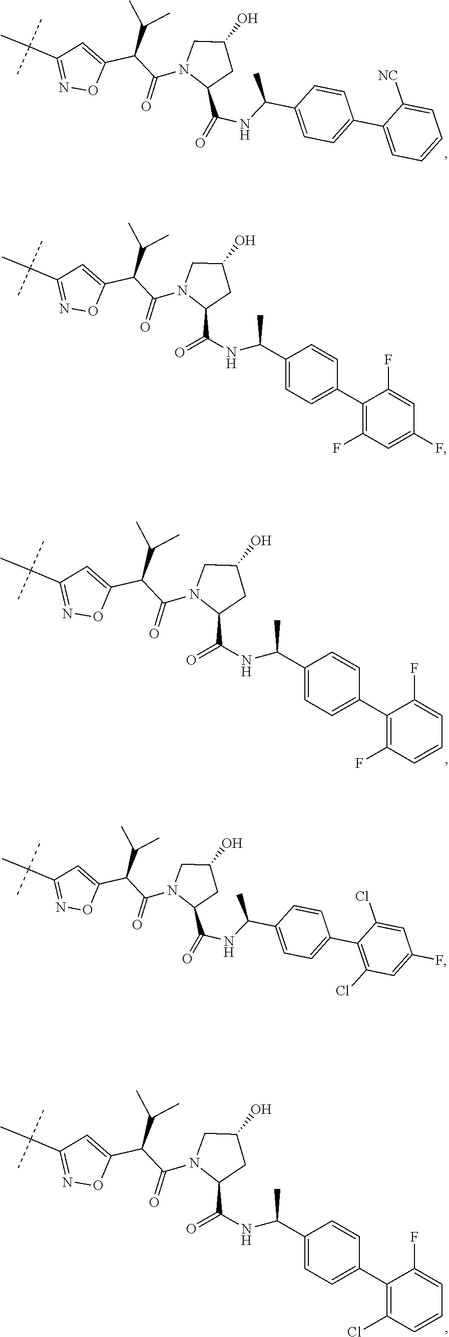

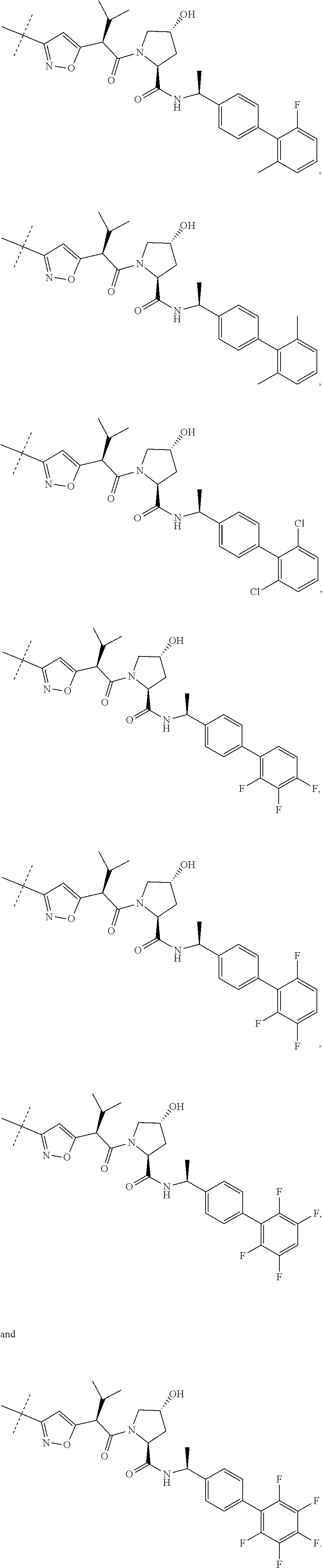

40. The conjugate of claim 38 or 39, wherein the target protein is a RAS protein.

41. The conjugate of claim 40, wherein the RAS protein is a KRAS mutant.

42. The conjugate of claim 41, wherein the KRAS mutant is selected from the group consisting of G12A, G12C, G12D, G12R, G12S, G12V, G13D, and G13C.

43. The conjugate of claim 38 or 39, wherein the protein binding ligand is a pan-KRAS inhibitor targeting two or more KRAS mutants.

44. The conjugate of claim 38 or 39, wherein the protein binding ligand is a KRAS mutant ligand.

45. A conjugate of Formula Q or a pharmaceutically acceptable salt thereof: 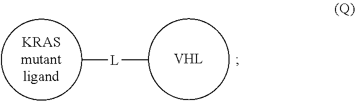
wherein the KRAS mutant ligand is capable of binding and/or inhibiting to a KRAS mutant protein; L is a bivalent linker that connects KRAS mutant ligand to a Von Hippel-Lindau (VHL) moiety; wherein VHL binds to a ubiquitin ligase; and
wherein the VHL ligand is a structure according to (i) or (ii): 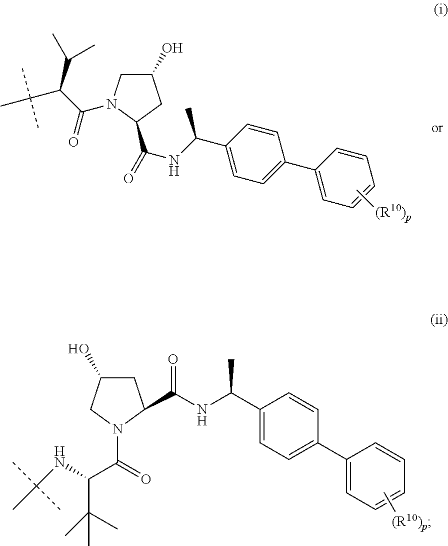
wherein p is an integer from 0 to 5; and
each R¹⁰ is independently selected from alkyl, —OH, cyano, and halogen.

46. The conjugate of claim 45, wherein each R¹⁰ is fluorine.

47. The conjugate of claim 45, wherein each R¹⁰ is fluorine and p is 1 to 5.

48. The conjugate of claim 45, wherein each R¹⁰ is chlorine.

49. The conjugate of claim 45, wherein each R¹⁰ is independently selected from chlorine and fluorine.

50. The conjugate of any one of claims 45 to 49, wherein L is: bond, NH, S, O, C(O), C(O)O, OC(O), NHC(O), C(O)NH, NHC(O)NH, NHC(NH)NH, C(S), substituted or unsubstituted alkylene, substituted or unsubstituted heteroalkylene, substituted or unsubstituted cycloalkylene, substituted or unsubstituted spirocycloalkylene, substituted or unsubstituted heterocycloalkylene, substituted or unsubstituted spiroheterocycloalkylene, substituted or unsubstituted arylene, substituted or unsubstituted heteroarylene or combinations thereof.

51. The conjugate of any one of claims 45 to 49, wherein -L- is selected from: 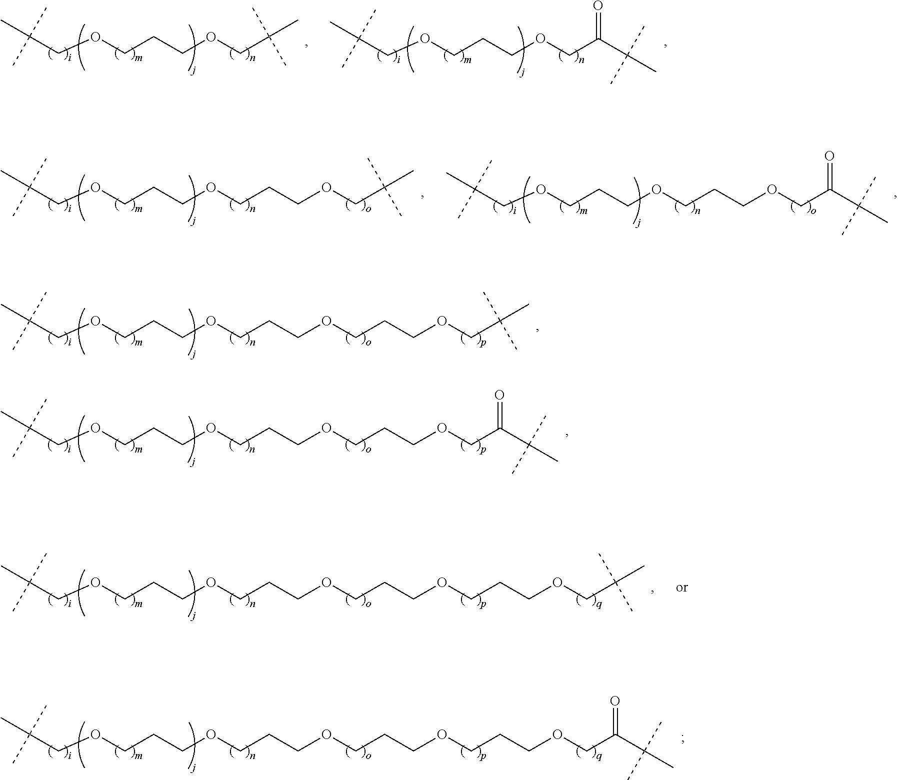
wherein any number of carbon or oxygen atoms of L are optionally exchanged with a heteroatom selected from NR^{L} S, or SO₂ atoms, where R^{L} is hydrogen or methyl; and i, j, m, n, o, p, q are integers independently selected from 0, 1, 2, 3, 4, 5, 6, 7, 8, 9 and 10; and wherein each carbon atom of L is optionally substituted with oxo (═O), OH, halo, NR₃R₄, C₁₋₄ alkyl, C₁₋₄ haloalkyl, C₁₋₄ alkoxy, C₃₋₄ cycloalkyl; wherein each L is attached to Z′ and Von Hippel-Lindau (VHL) moiety in either orientation.

52. The conjugate of any one of claims 45 to 49, wherein -L- is selected from: 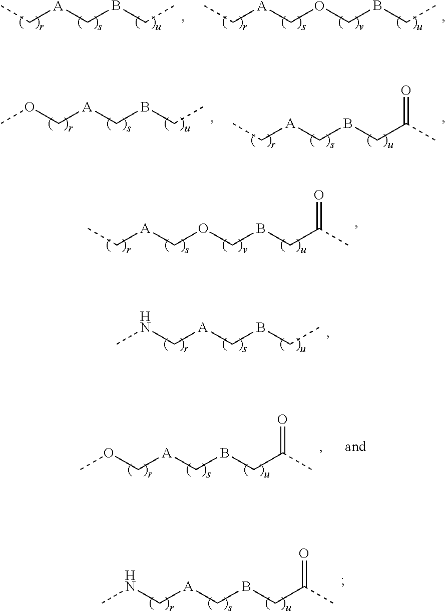
wherein each A and B are independently selected from absent, a 3 to 10 membered cycloalkylene, 4- to 10-membered heterocycloalkylene, 5- to 10-membered heteroarylene and 6- to 10-membered arylene, wherein any number of carbon or oxygen atoms of L are optionally exchanged with a heteroatom selected from NR^{L}, S or SO₂ atoms, where R^{L} is hydrogen or methyl; r, s, u, v are integers independently selected from 0, 1, 2, 3, 4, 5, 6, 7, 8, 9 and 10; wherein each carbon atom of L is optionally substituted with oxo (═O), OH, halo, NR₃R₄, C₁₋₄ alkyl, C₁₋₄ haloalkyl, C₁₋₄ alkoxy, C₃₋₄ cycloalkyl; wherein each L is attached to Z′ and ubiquitin binding moiety (UBM) in either orientation; and
when A and B are a 3- to 10-membered cycloalkyl or 4- to 10-membered heterocycloalkyl, they are incorporated at any two independent ring atoms, or at the same ring atom.

53. The conjugate of any one of claims 45 to 49, wherein -L- is selected from:

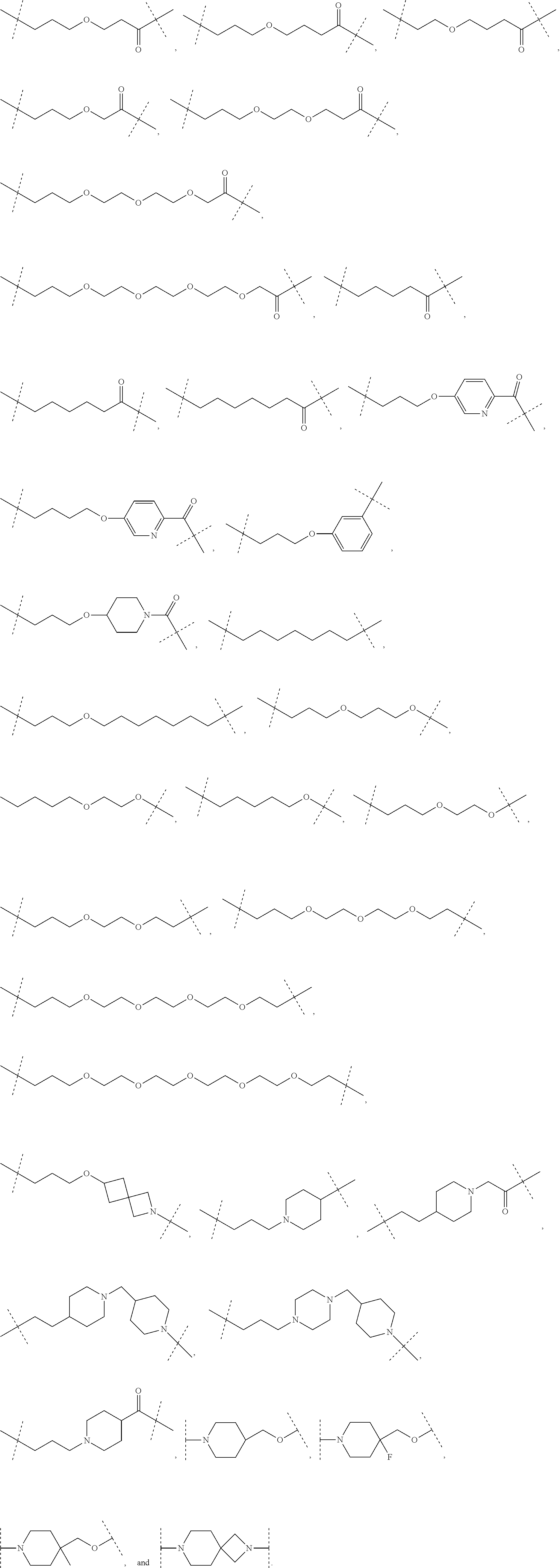

54. The conjugate of any one of claims 45 to 49, wherein -L- is a structure according to formula (L0): 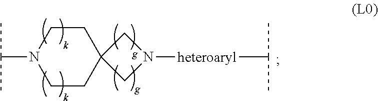
wherein each k is independently 0 or 1; and,
each g is independently 1 or 2.

55. The conjugate of any one of claims 45 to 49, wherein -L- is a structure according to formula (L1) or (L2): 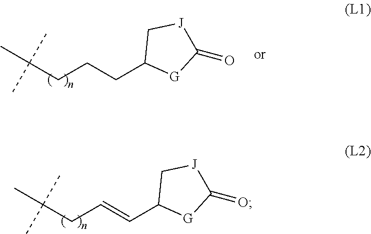
J is null, —CH₂—, O, —OCH₂—, —CH₂O—, —CH₂CH₂—;
G is a nitrogen attached to ubiquitin binding motiety (UBM); and
n is an integer selected from 0, 1, and 2.

56. The conjugate of any one of claims 45 to 49, wherein -L- is selected from:

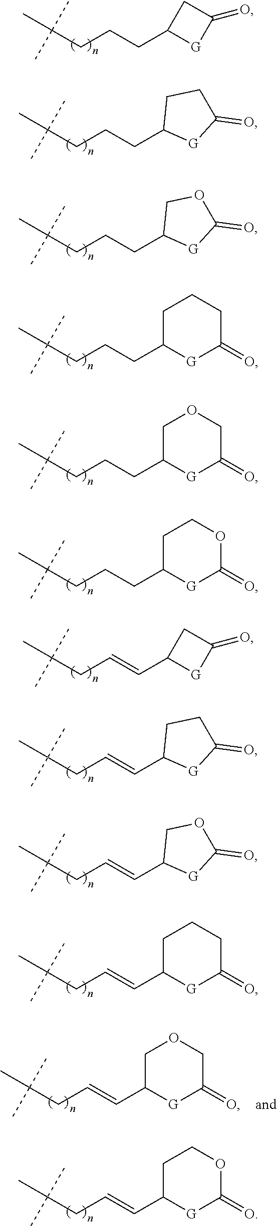

57. The conjugate of claim 55 or 56, wherein n is 1.

58. The conjugate of any one of claims 45 to 57, wherein the target protein is a RAS protein.

59. The conjugate of claim 58, wherein the RAS protein is a KRAS mutant.

60. The conjugate of claim 59, wherein the KRAS mutant is selected from the group consisting of G12A, G12C, G12D, G12R, G12S, G12V, G13D, and G13C.

61. The conjugate of claim 45 having the structure or pharmaceutically acceptable salt thereof selected from: 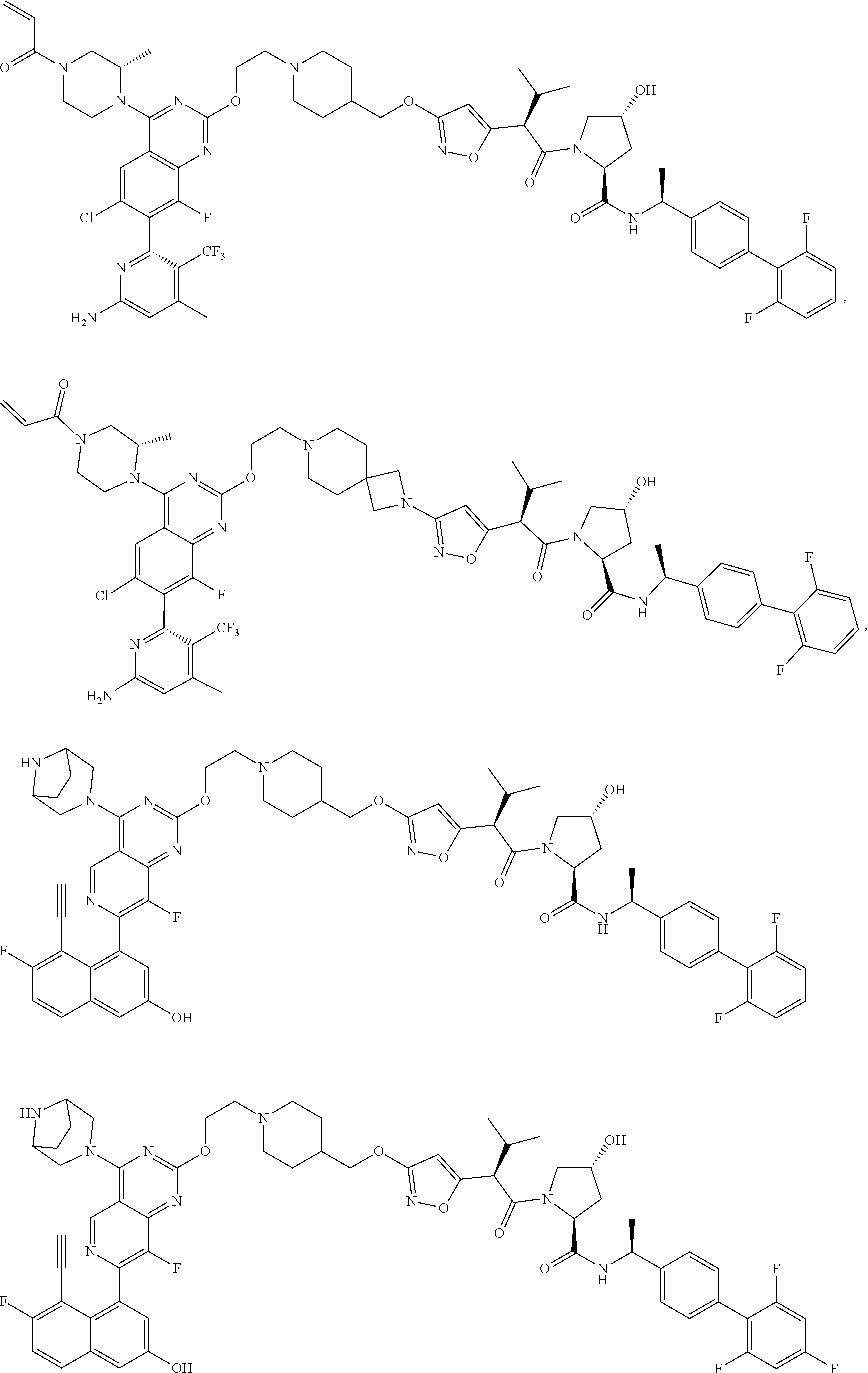 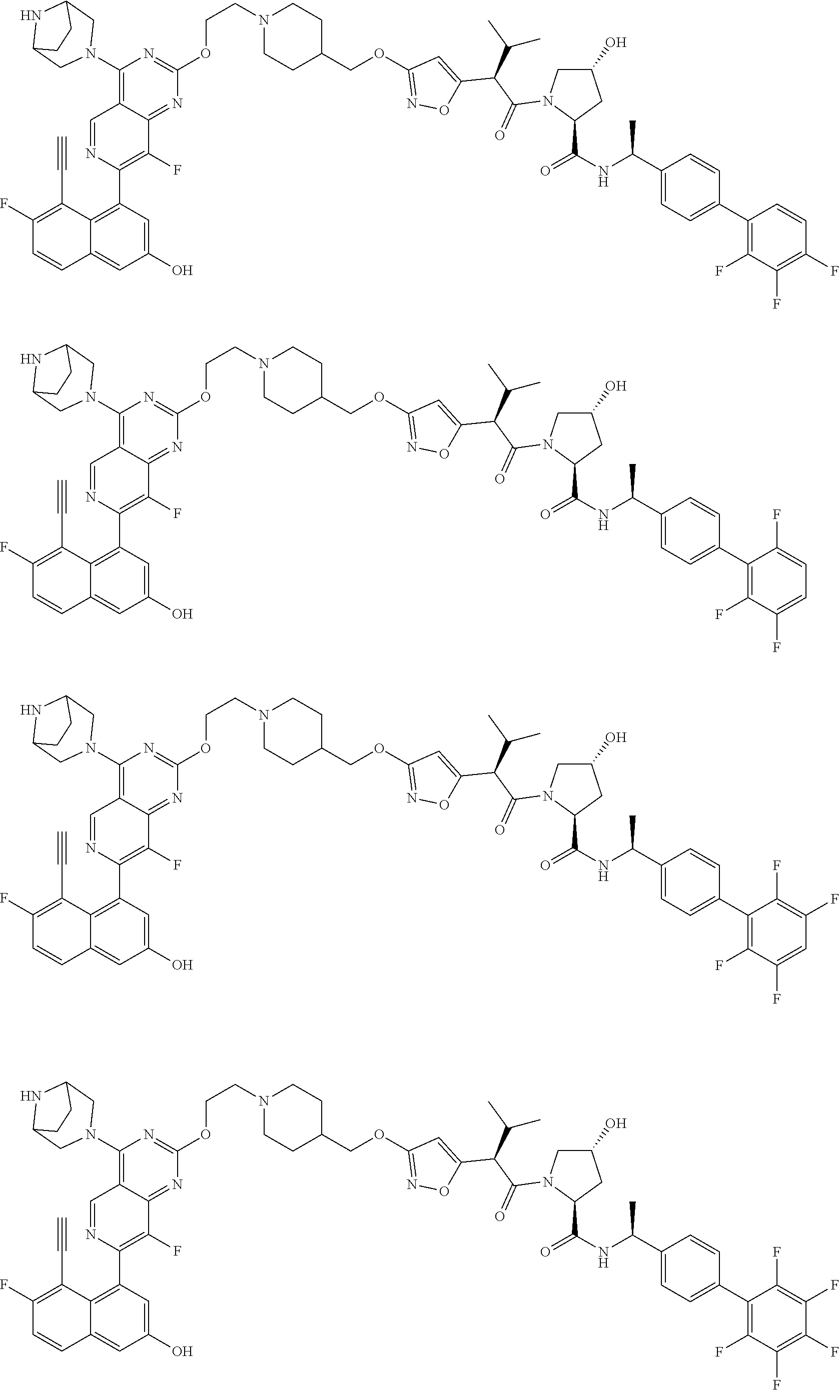 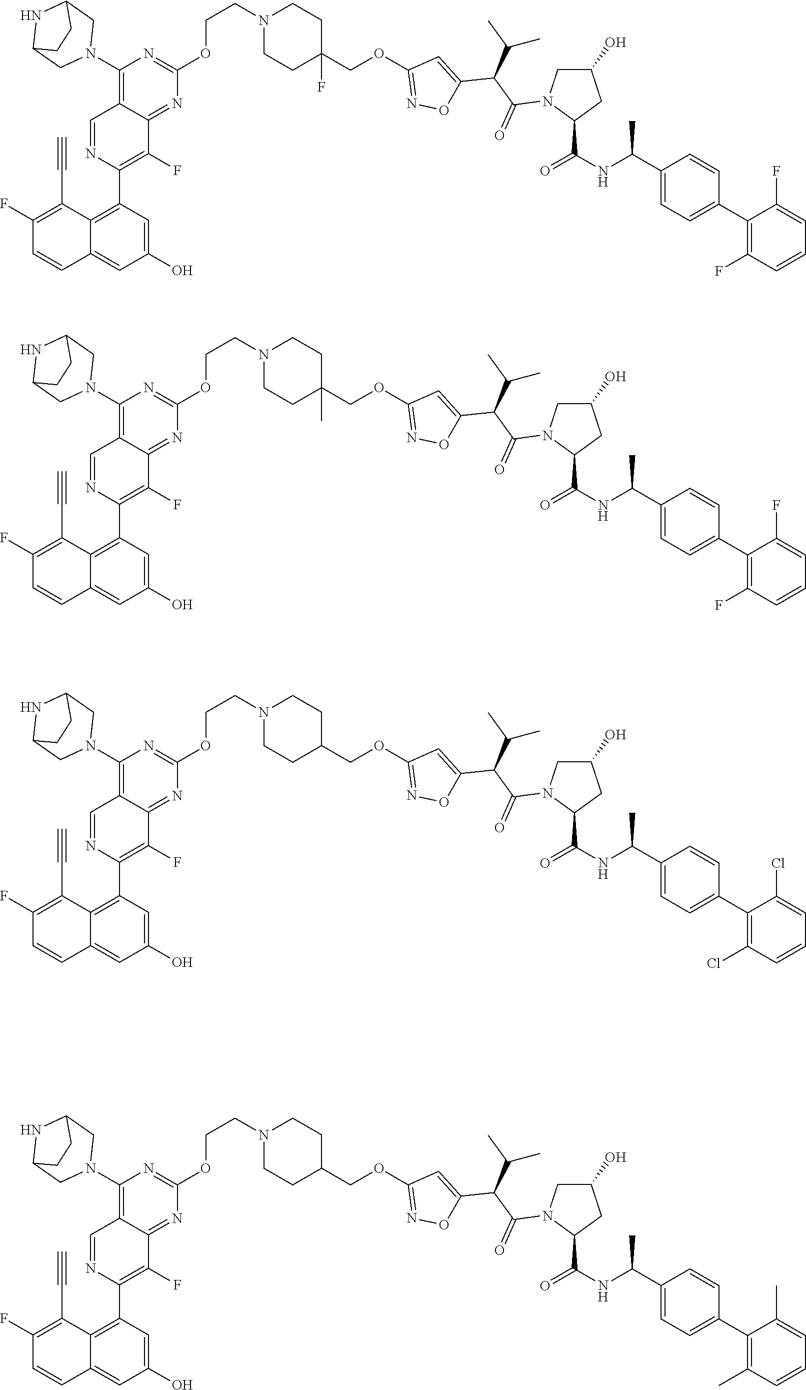 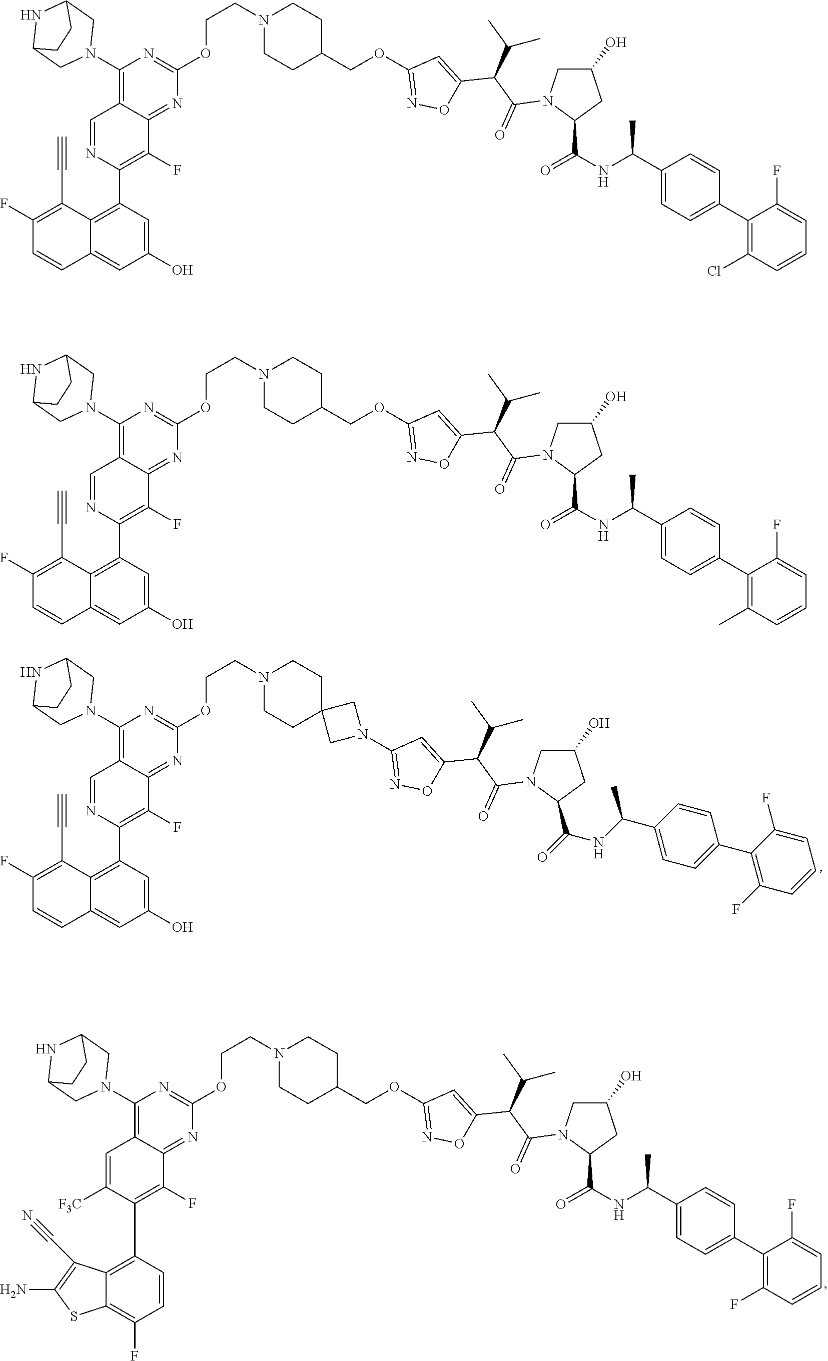 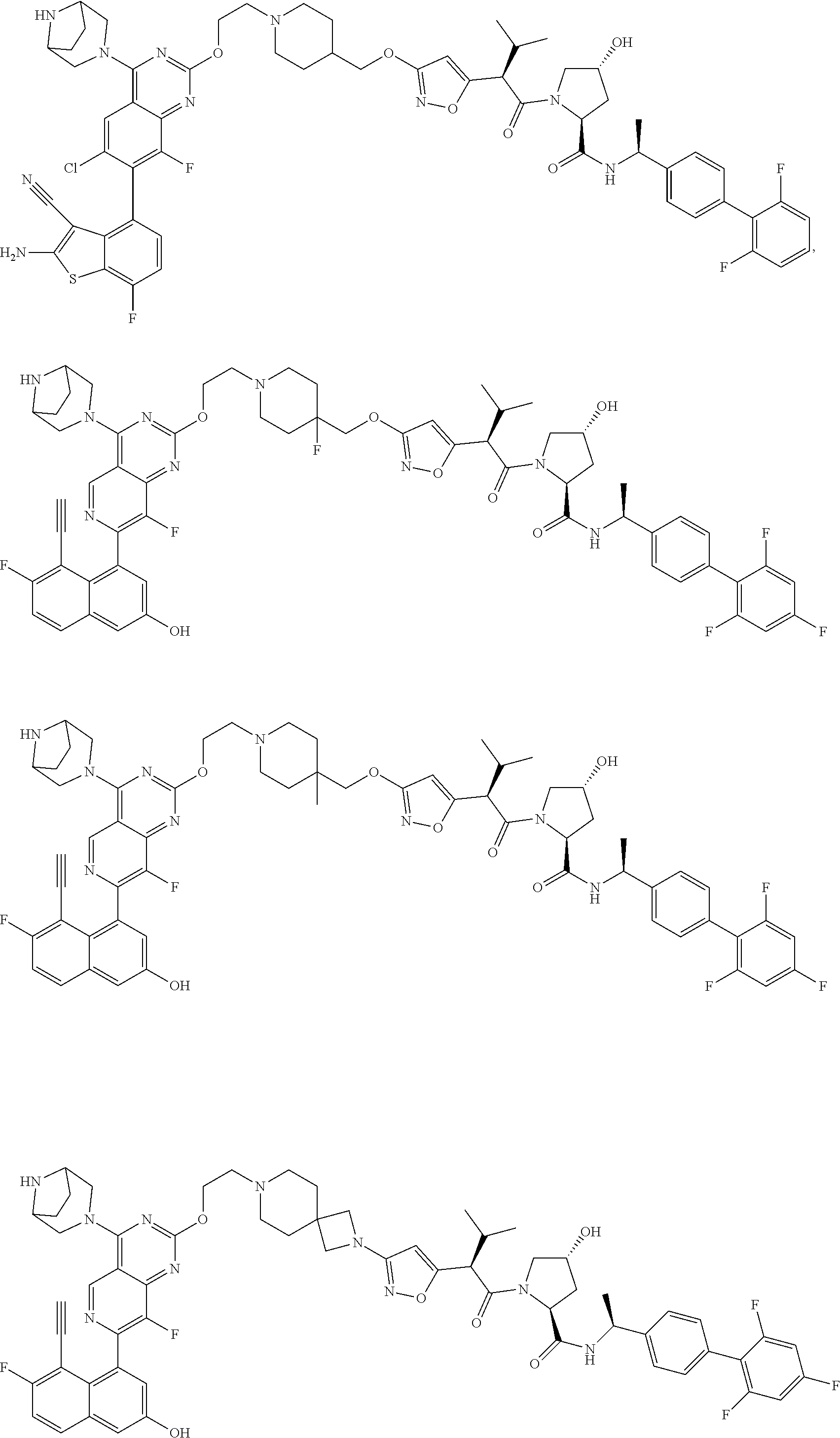 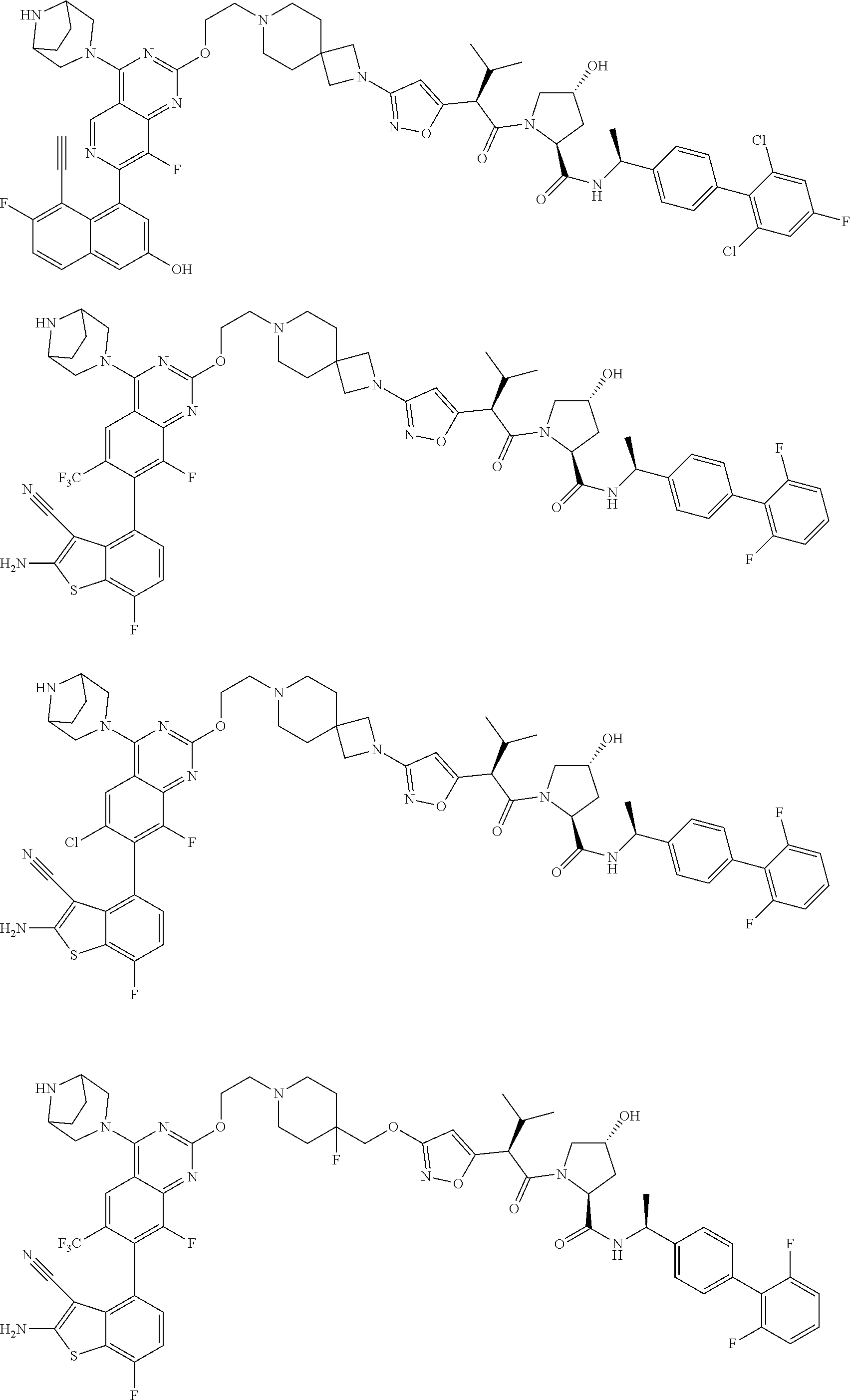 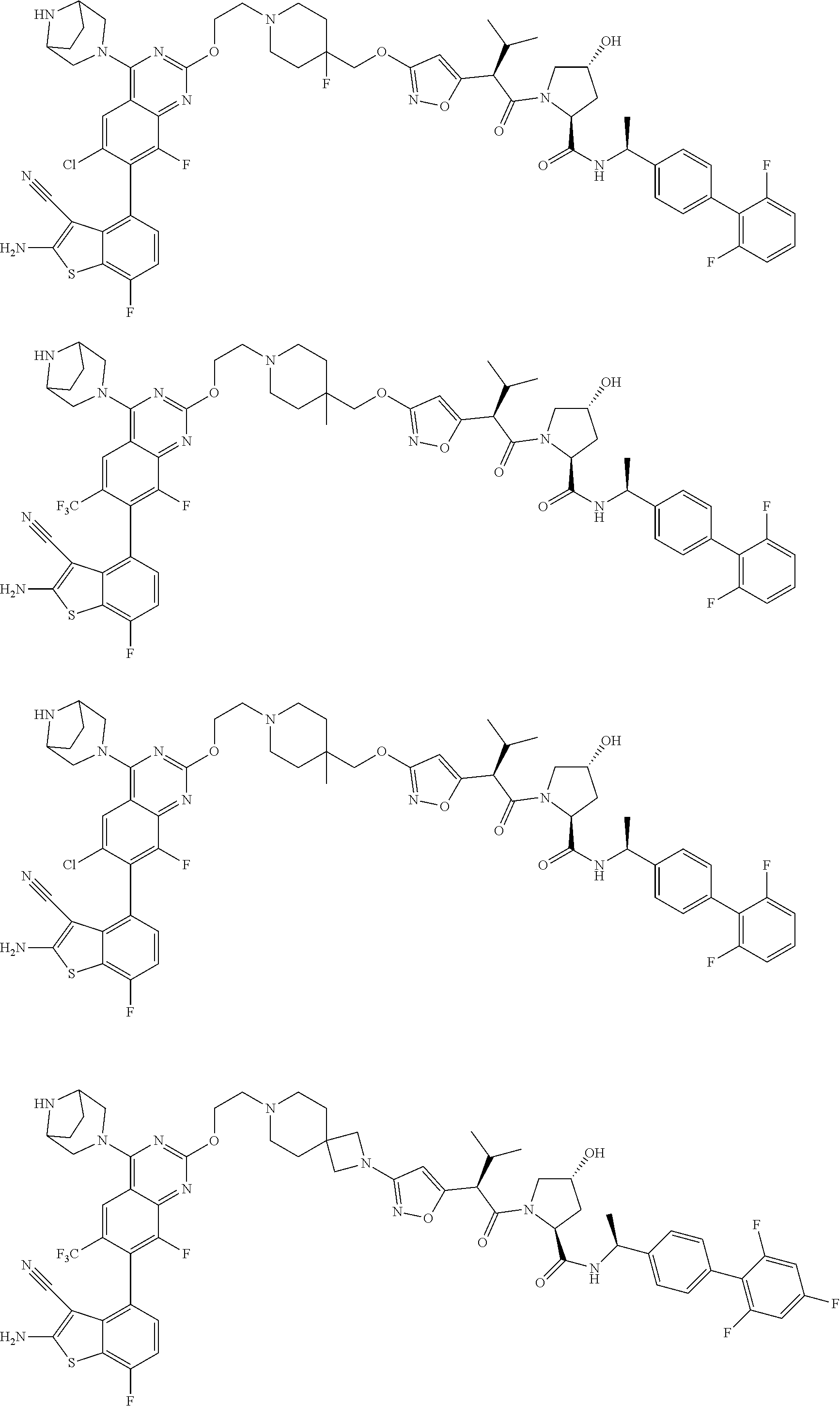 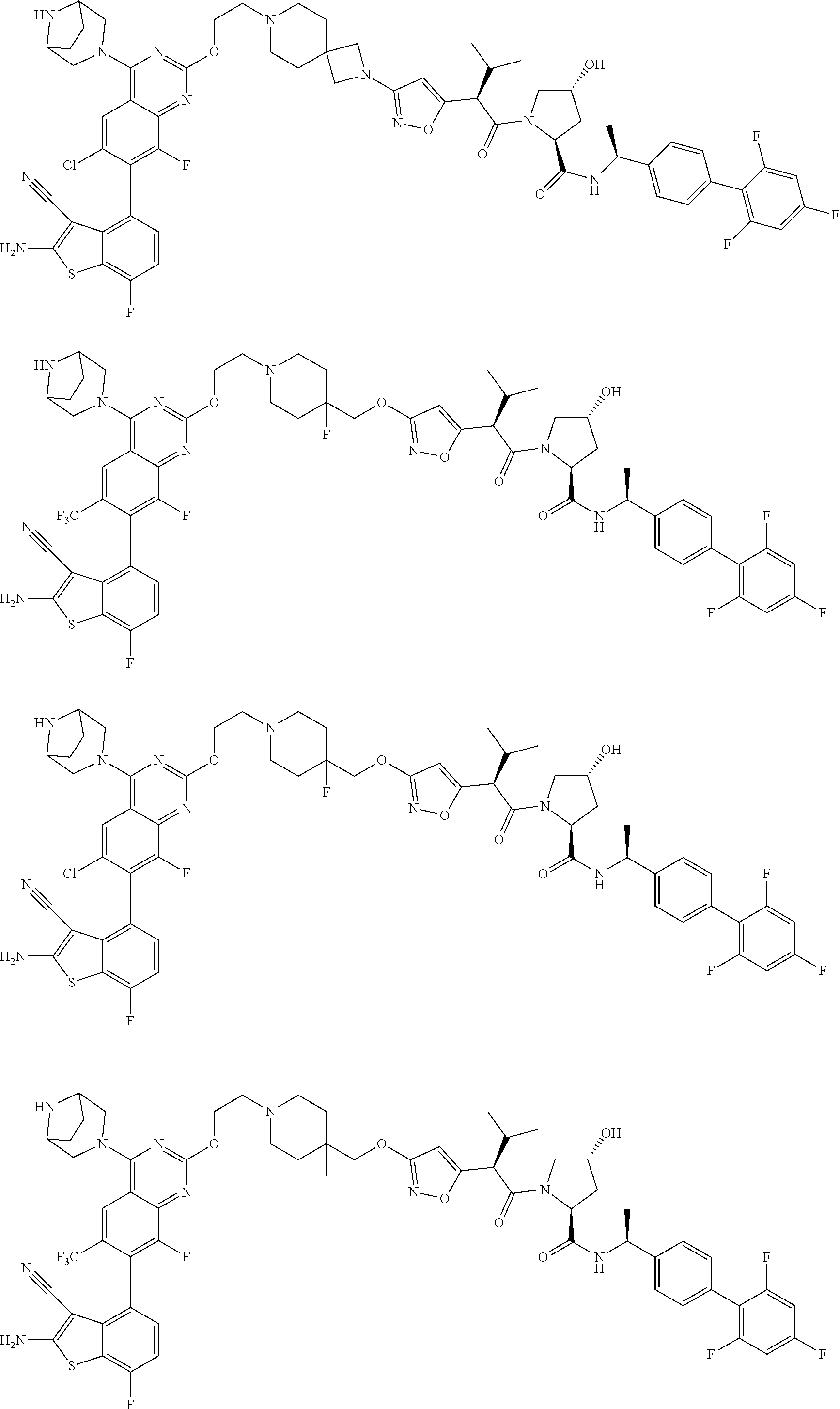 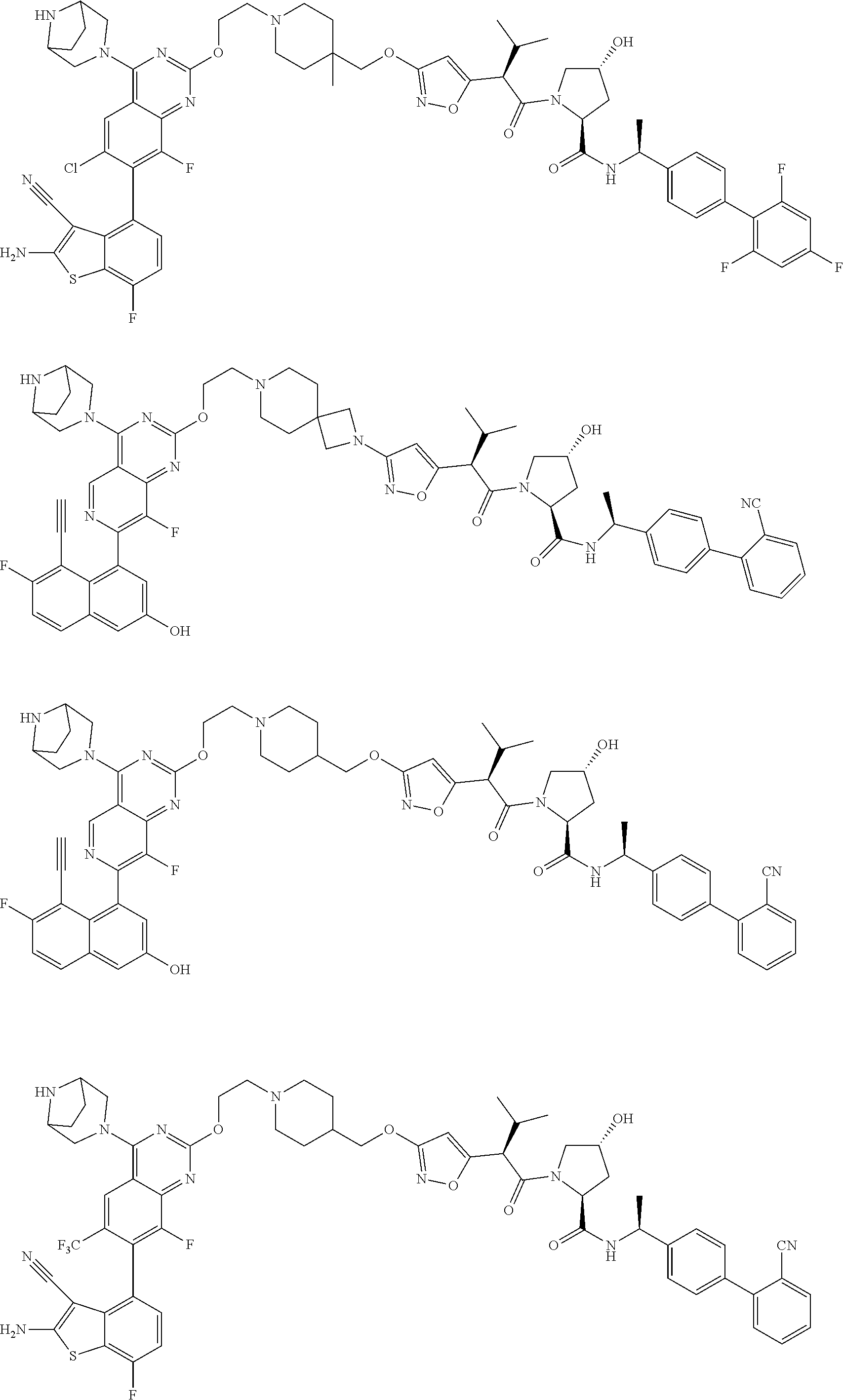 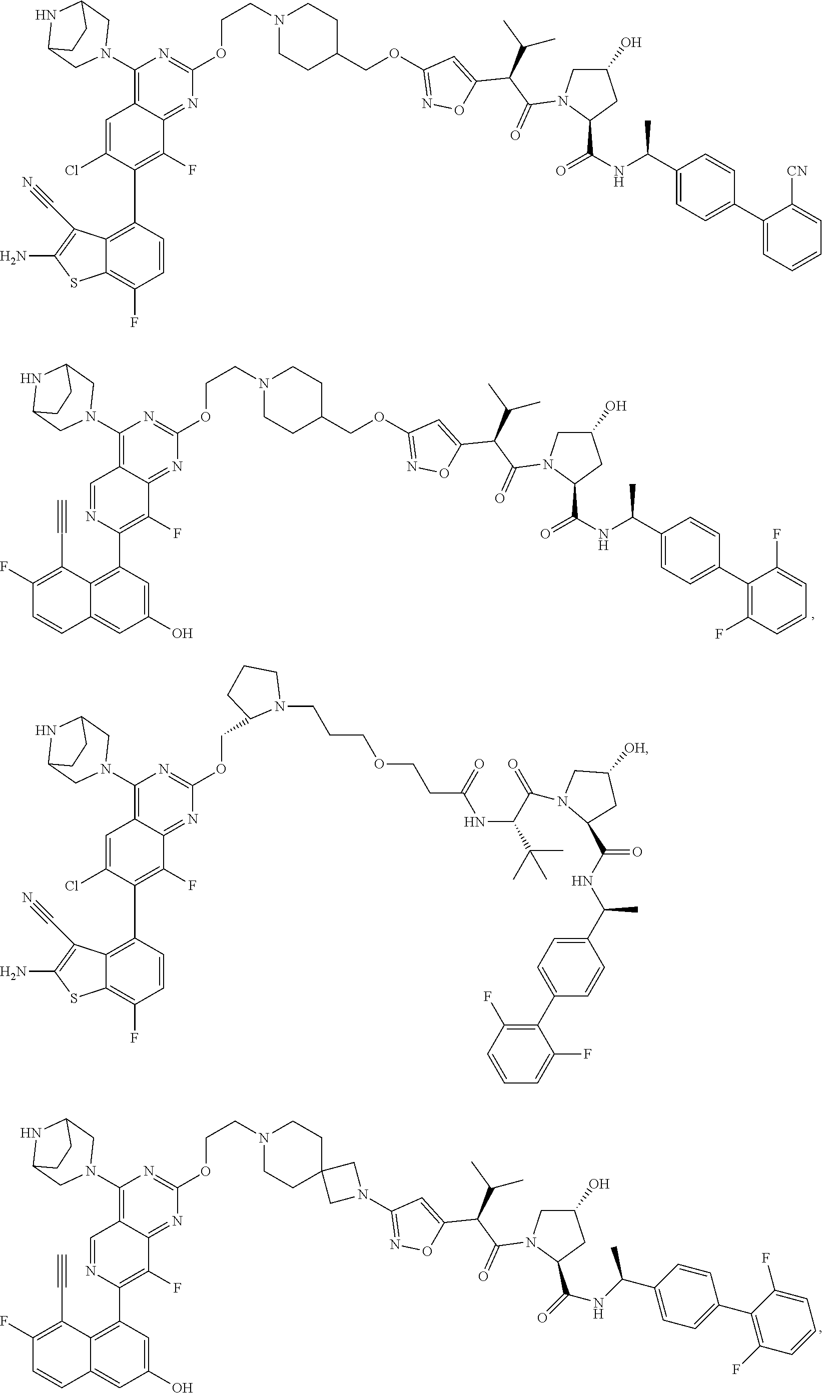 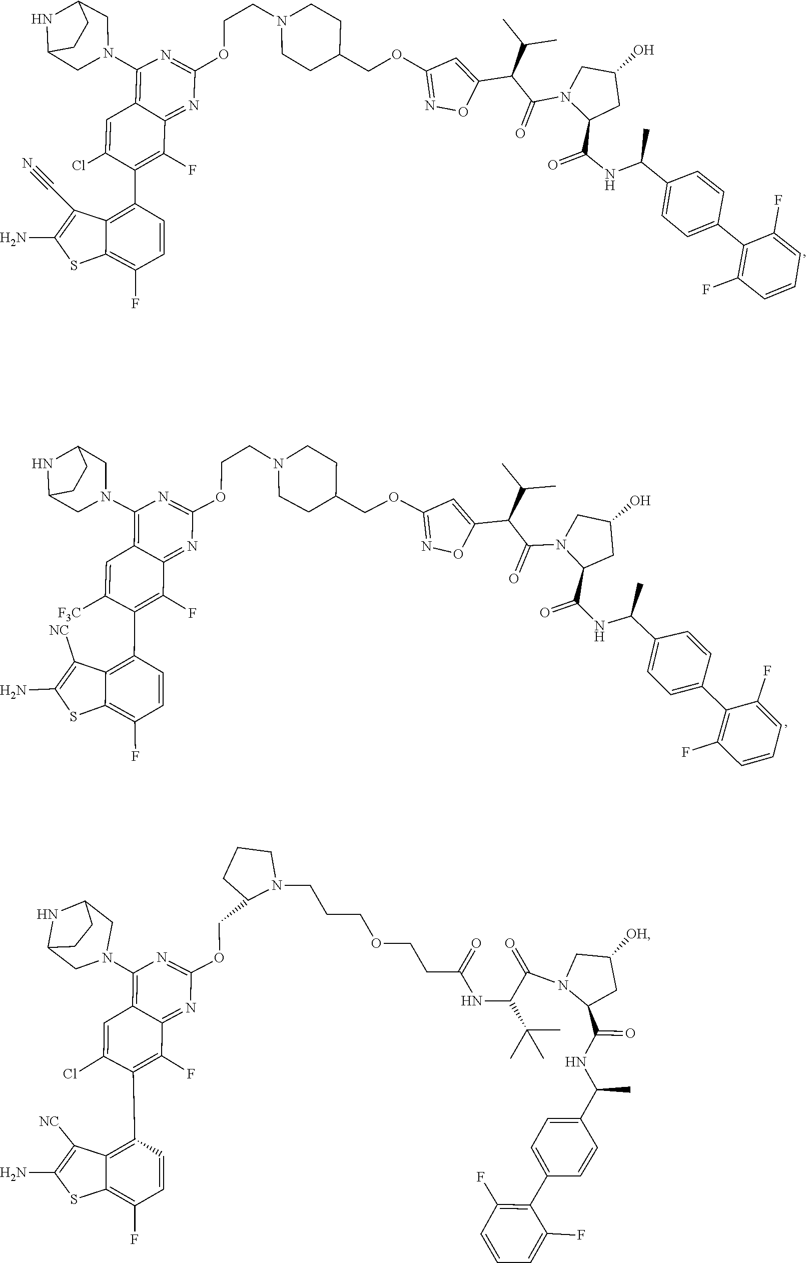 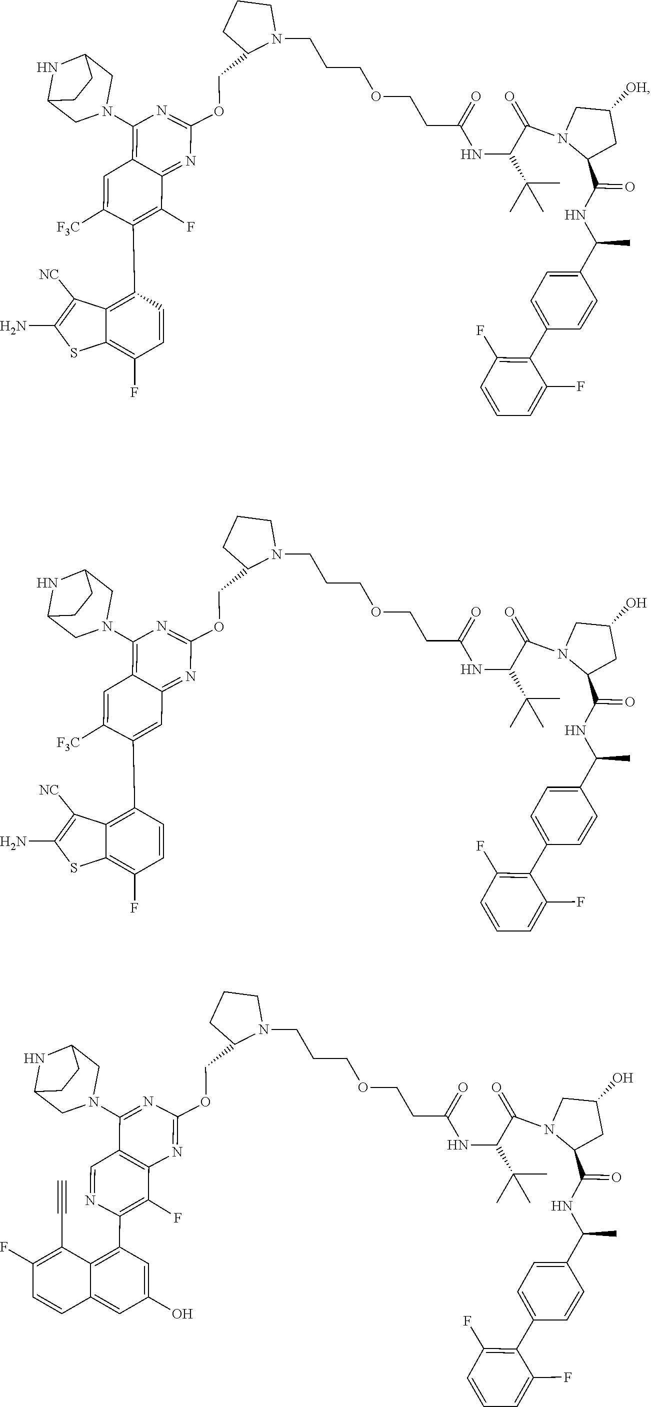 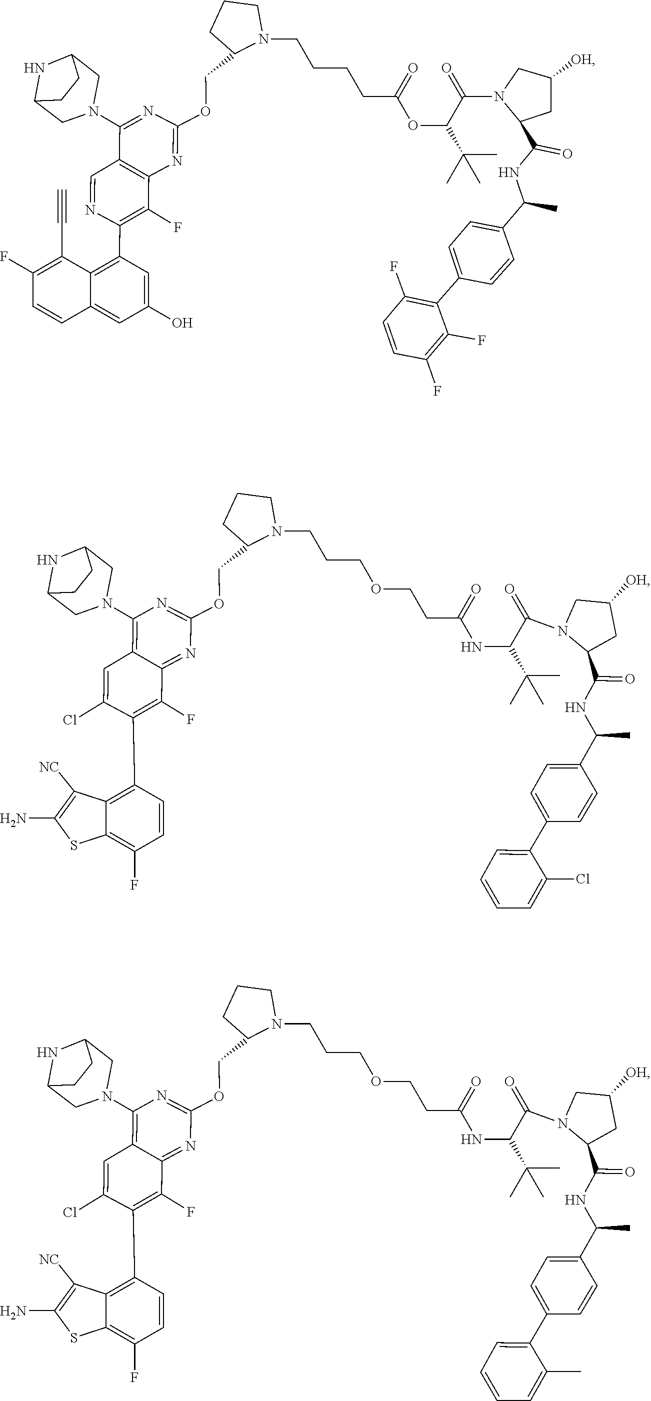 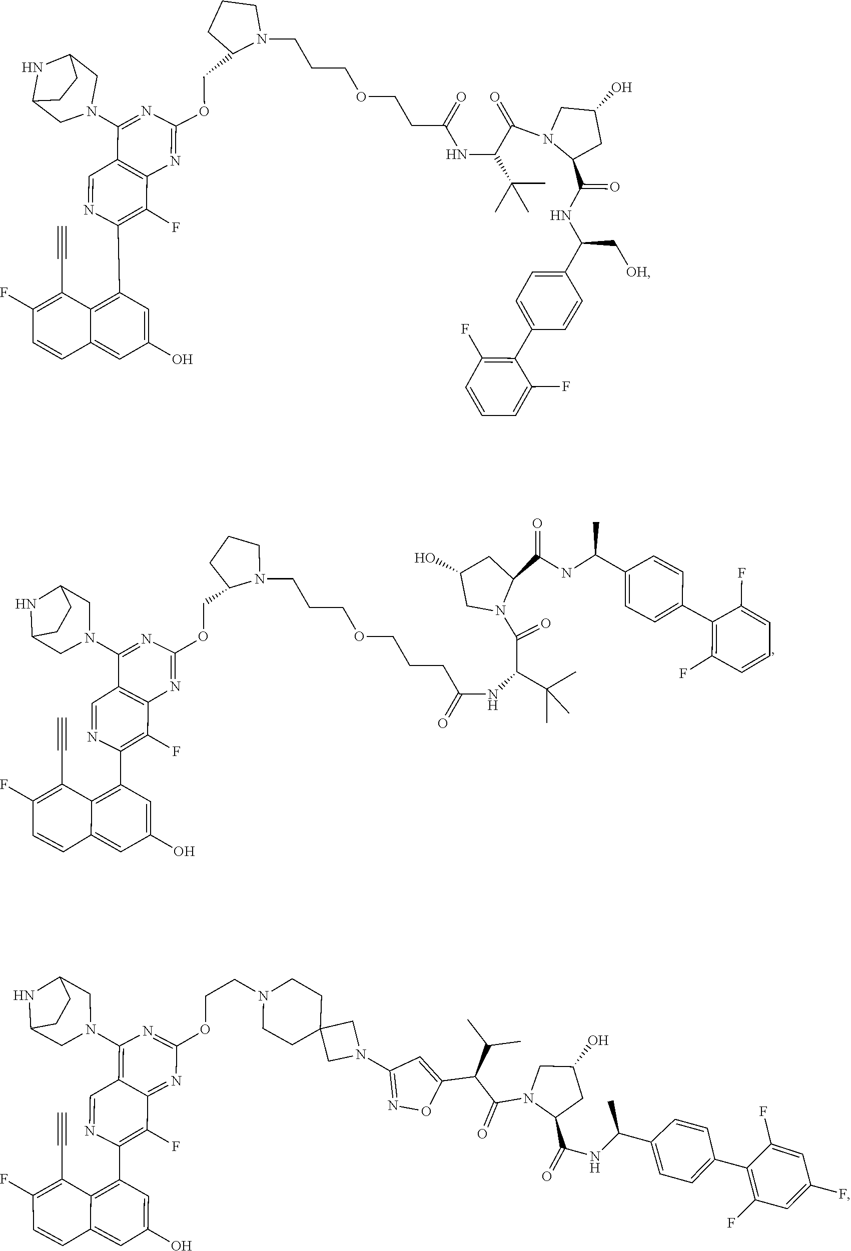 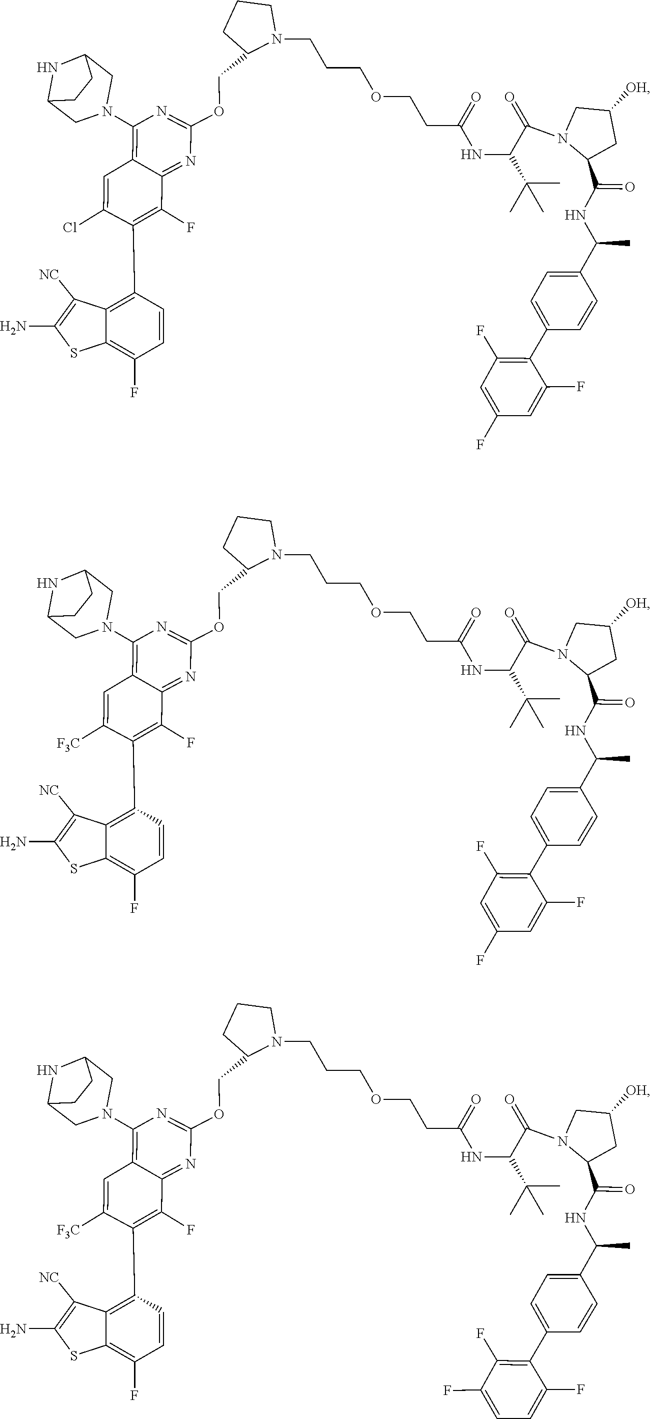 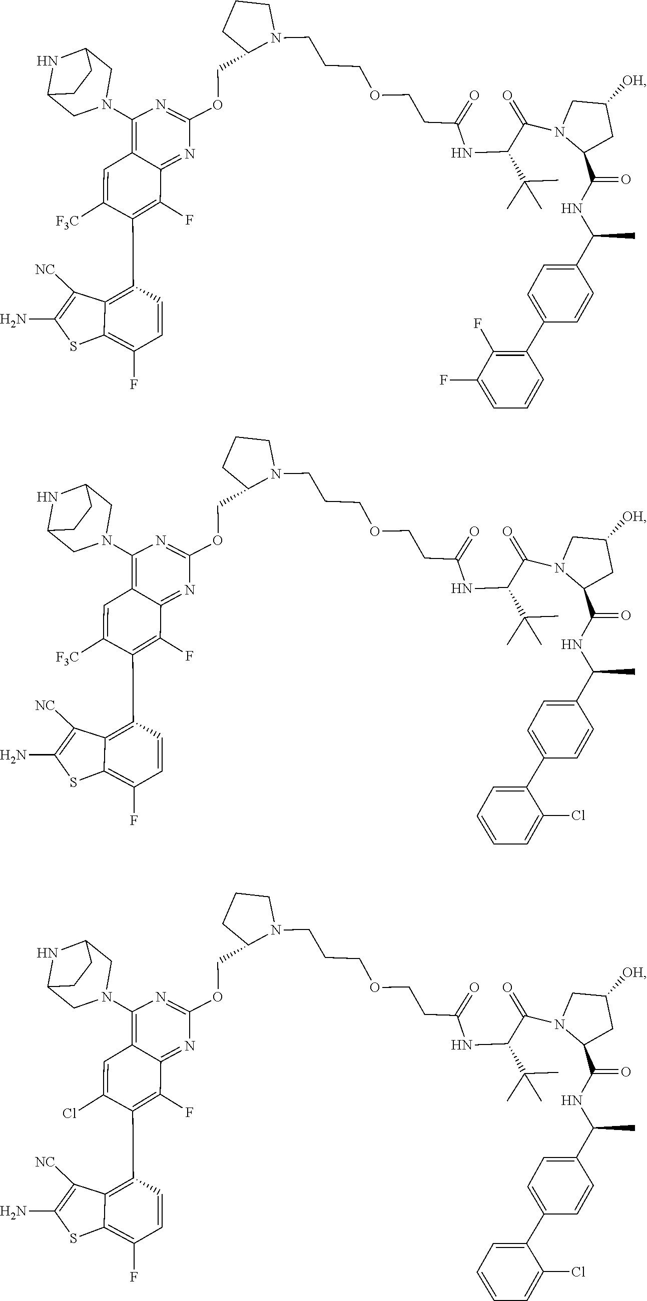 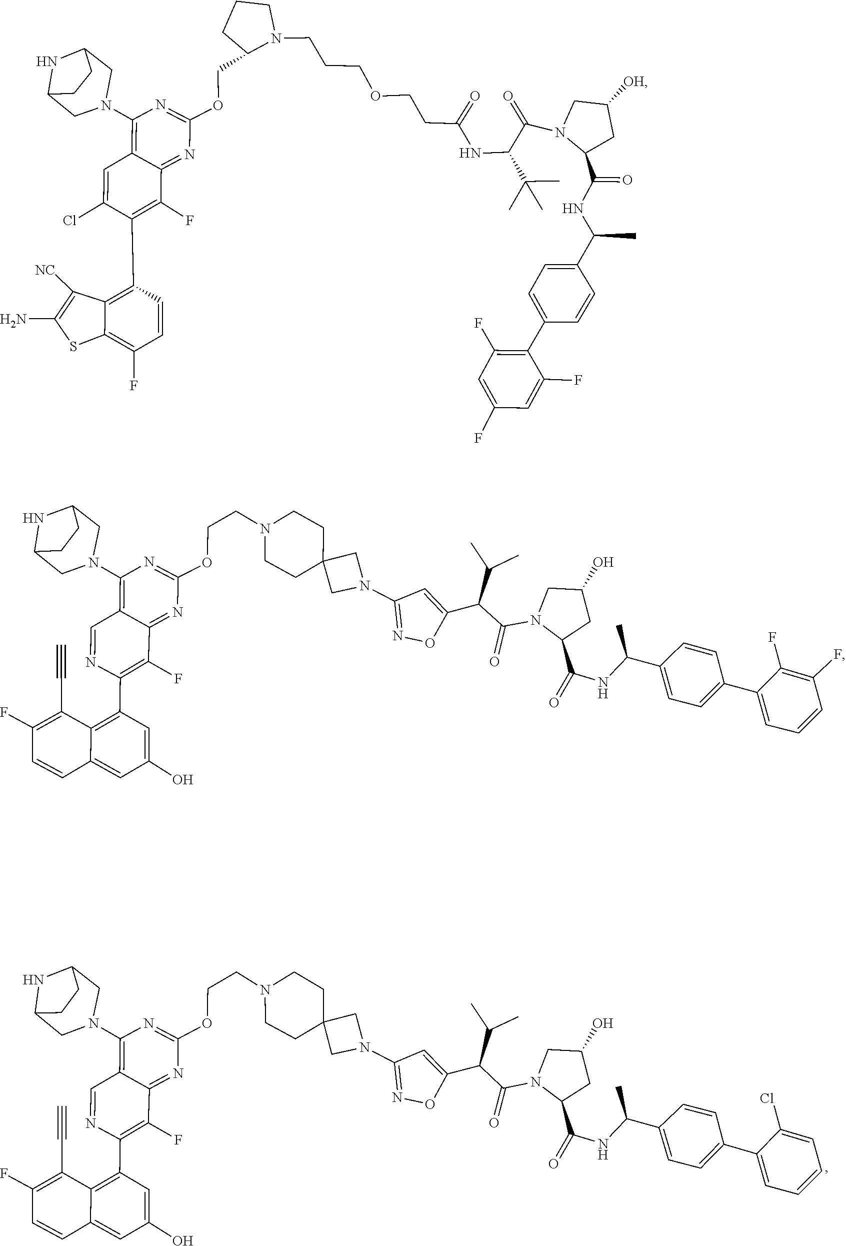 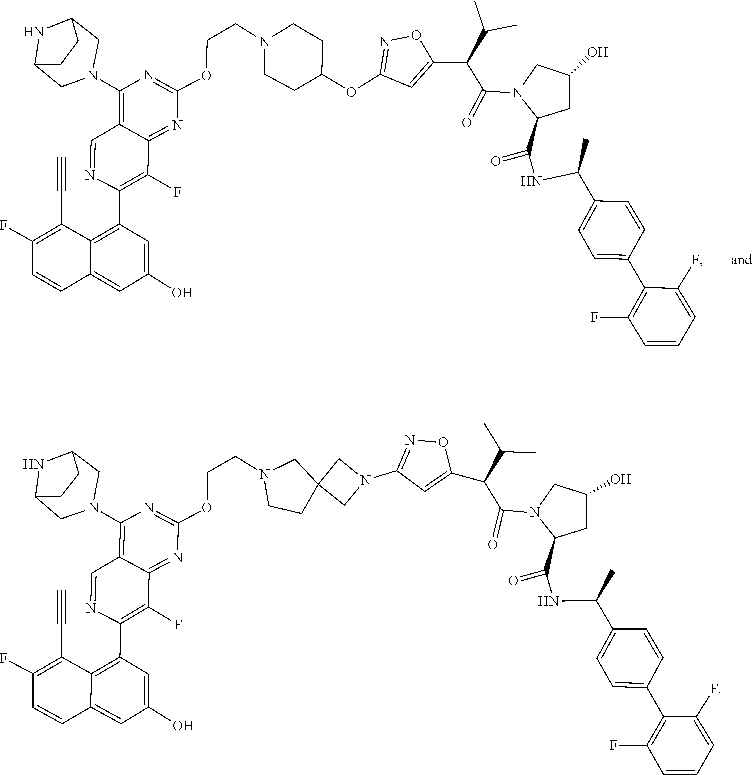 [SMILES: C#Cc1c(F)ccc2cc(O)cc(-c3ncc4c(N5CC6CCC(C5)N6)nc(OCCN5CCC(Oc6cc([C@H](C(=O)N7C[C@H](O)C[C@H]7C(=O)N[C@@H](C)c7ccc(-c8c(F)cccc8F)cc7)C(C)C)on6)CC5)nc4c3F)c12 ; C#Cc1c(F)ccc2cc(O)cc(-c3ncc4c(N5CC6CCC(C5)N6)nc(OCCN5CCC6(C5)CN(c5cc([C@H](C(=O)N7C[C@H](O)C[C@H]7C(=O)N[C@@H](C)c7ccc(-c8c(F)cccc8F)cc7)C(C)C)on5)C6)nc4c3F)c12]
